(19) United States
(12) Patent Application Publication
(10) Pub. No.: US 2024/0114776 A1
(43) Pub. Date: Apr. 4, 2024

(54) COMPOUND AND ORGANIC LIGHT EMITTING DEVICE COMPRISING THE SAME

(71) Applicant: LG CHEM, LTD., Seoul (KR)

(72) Inventors: Minjun Kim, Daejeon (KR); Dong Hoon Lee, Daejeon (KR); Sang Duk Suh, Daejeon (KR); Donghee Kim, Daejeon (KR)

(21) Appl. No.: 18/265,894

(22) PCT Filed: Feb. 24, 2022
(86) PCT No.: PCT/KR2022/002695

§ 371 Date: Jun. 7, 2023

(30) Foreign Application Priority Data
Feb. 24, 2021 (KR) 10-2021-0024902

(51) Int. Cl. H10K 85/60; C07D 263/60; C09K 11/06; C07D 413/12; C07D 413/14

(52) U.S. Cl. CPC H10K 85/636; C07D 263/60; C09K 11/06; H10K 85/633; C07D 413/12; C07D 413/14; H10K 50/181

(57) ABSTRACT

A compound of Chemical Formula 1 or 2 and an organic light emitting device including the same are provided. The compound, when used as a material for an organic material layer of an organic light emitting device, provides improved efficiency, low driving voltage, and enhanced lifespan of the organic light emitting device.

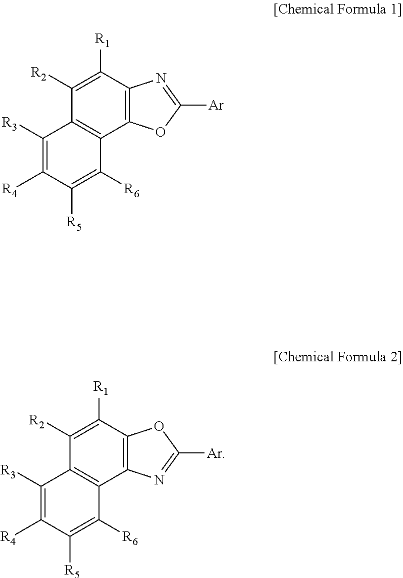

### CROSS-REFERENCE TO RELATED APPLICATION(S)

This application is a National Phase entry pursuant to 35 U.S.C. § 371 of International Application No. PCT/KR2022/002695 filed on Feb. 24, 2022, and claims priority to and the benefit of Korean Patent Application No. 10-2021-0024902 filed on Feb. 24, 2021, the disclosures of which are incorporated herein by reference in their entirety.

### FIELD

The present disclosure relates to a novel compound and an organic light emitting device including the same.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer may be formed of a hole injection layer, a hole transport layer, a light emitting layer, an electron transport layer, an electron injection layer, and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuing need for the development of new materials for the organic materials used in the organic light emitting devices as described above.

### RELATED ART

(Patent Literature 0001) Korean Unexamined Patent Publication No. 10-2000-0051826

### SUMMARY

The present disclosure relates to a novel compound and an organic light emitting device including the same.

In the present disclosure, there is provided a compound represented by the following Chemical Formula 1 or 2:

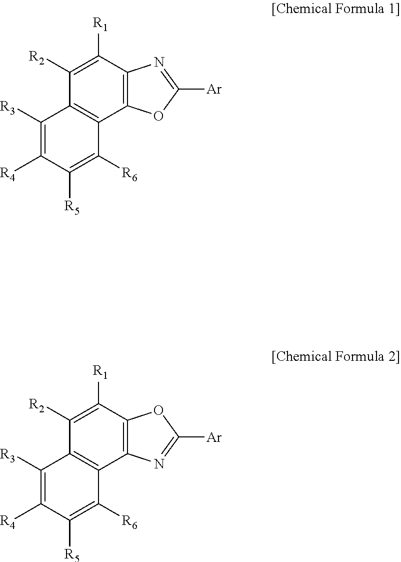

in Chemical Formulae 1 and 2,
Ar is a substituted or unsubstituted C₆₋₆₀ aryl, and
one of R₁ to R₆ is the following Chemical Formula 3, and the rest are each independently hydrogen, or deuterium,

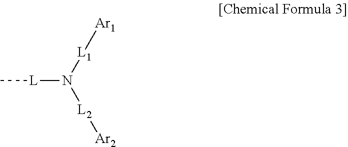

in Chemical Formula 3,
L is a single bond, a substituted or unsubstituted C₆₋₆₀ arylene, or a substituted or unsubstituted C₂₋₆₀ heteroarylene containing at least one selected from the group consisting of N, O and S,
L₁ and L₂ are each independently a single bond, a substituted or unsubstituted C₆₋₆₀ arylene, or a substituted or unsubstituted C₂₋₆₀ heteroarylene containing at least one selected from the group consisting of N, O and S, and
Ar₁ and Ar₂ are each independently a substituted or unsubstituted C₆₋₆₀ aryl, or a substituted or unsubstituted C₂₋₆₀ heteroaryl containing at least one selected from the group consisting of N, O and S,
provided that when R₅ or R₆ is the substituent represented by the Chemical Formula 3,
L₁ is a substituted or unsubstituted C₆₋₆₀ arylene, and Ar is a substituted or unsubstituted C₆₋₆₀ aryl; or
L₁ is a single bond, or a substituted or unsubstituted C₆₋₆₀ arylene, and Ar is a substituted or unsubstituted C₂₋₆₀ heteroaryl containing at least one selected from the group consisting of N, O and S.

In addition, there is provided an organic light emitting device including: a first electrode; a second electrode that is disposed to face the first electrode; and an organic material layer including one or more layers between the first electrode and the second electrode, wherein one or more layers of the organic material layer include the compound represented by the Chemical Formula 1 or 2.

### Advantageous Effects

The compound represented by the Chemical Formula 1 or Chemical Formula 2 may be used as a material for an organic material layer of an organic light emitting device, and may improve efficiency, low driving voltage, and/or lifespan of the organic light emitting device. In particular, the compound represented by the Chemical Formula 1 or 2 may be used as a material for hole injection, hole transport, hole injection and transport, light emission, electron transport, or electron injection.

### BRIEF DESCRIPTION OF THE DRAWINGS

FIG. 1 shows an example of an organic light emitting device including a substrate 1, an anode 2, a light emitting layer 3, and a cathode 4.

FIG. 2 shows an example of an organic light emitting device including a substrate 1, an anode 2, a hole injection layer 5, a hole transport layer 6, an electron blocking layer 7, a light emitting layer 3, an electron transport layer 8, and a cathode 4.

### DETAILED DESCRIPTION OF THE EMBODIMENTS

Hereinafter, embodiments of the present disclosure will be described in more detail to facilitate understanding of the invention.

As used herein, the notation

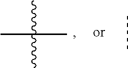

or means a bond linked to another substituent group.

As used herein, the term “substituted or unsubstituted” means being unsubstituted or substituted with one or more substituents selected from the group consisting of deuterium; a halogen group; a nitrile group; a nitro group; a hydroxyl group; a carbonyl group; an ester group; an imide group; an amino group; a phosphine oxide group; an alkoxy group; an aryloxy group; an alkylthioxy group; an arylthioxy group; an alkylsulfoxy group; an arylsulfoxy group; a silyl group; a boron group; an alkyl group; a cycloalkyl group; an alkenyl group; an aryl group; an aralkyl group; an aralkenyl group; an alkylaryl group; an alkylamine group; an aralkylamine group; a heteroarylamine group; an arylamine group; an arylphosphine group; and a heterocyclic group containing at least one of N, O and S atoms, or being unsubstituted or substituted with a substituent in which two or more substituents of the above-exemplified substituents are connected. For example, “a substituent in which two or more substituents are connected” may be a biphenyl group. Namely, a biphenyl group may be an aryl group, or it may also be interpreted as a substituent in which two phenyl groups are connected.

In the present disclosure, the carbon number of a carbonyl group is not particularly limited, but is preferably 1 to 40. Specifically, the carbonyl group may be a compound having the following structural formulae, but is not limited thereto.

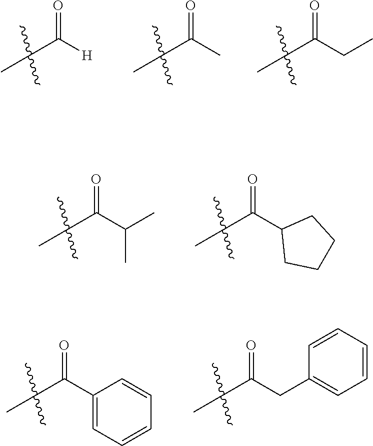

In the present disclosure, an ester group may have a structure in which oxygen of the ester group is substituted by a straight-chain, branched-chain, or cyclic alkyl group having 1 to 25 carbon atoms, or an aryl group having 6 to 25 carbon atoms. Specifically, the ester group may be a compound having the following structural formulae, but is not limited thereto.

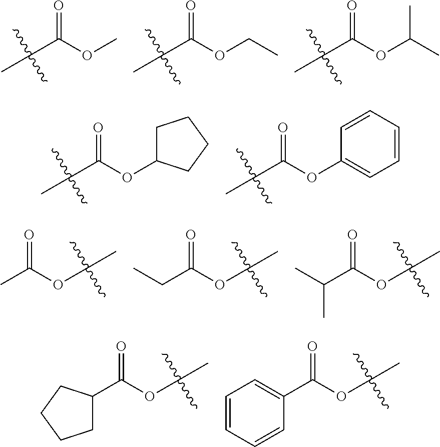

In the present disclosure, the carbon number of an imide group is not particularly limited, but is preferably 1 to 25. Specifically, the imide group may be a compound having the following structural formulae, but is not limited thereto.

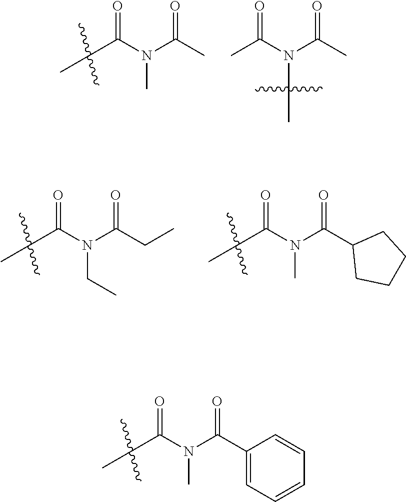

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but is not limited thereto.

In the present disclosure, examples of a halogen group include fluorine, chlorine, bromine, or iodine.

In the present disclosure, the alkyl group may be straight-chain, or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present disclosure, the alkenyl group may be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it may be a monocyclic aryl group or a polycyclic aryl group. According to one embodiment, the carbon number of the aryl group is 6 to 30. According to one embodiment, the carbon number of the aryl group is 6 to 20. The monocyclic aryl group includes a phenyl group, a biphenyl group, a terphenyl group and the like, but is not limited thereto. The polycyclic aryl group includes a naphthyl group, an anthracenyl group, a phenanthryl group, a pyrenyl group, a perylenyl group, a chrysenyl group, a fluorenyl group or the like, but is not limited thereto.

In the present disclosure, a fluorenyl group may be substituted, and two substituents may be bonded to each other to form a spiro structure. In the case where the fluorenyl group is substituted,

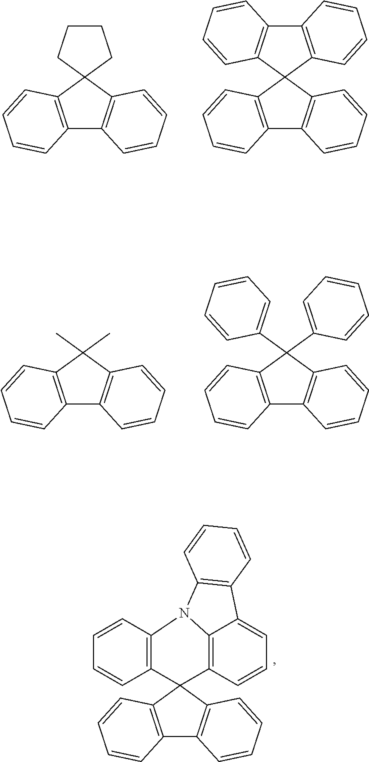

and the like can be formed. However, the structure is not limited thereto.

In the present disclosure, a heterocyclic group is a heterocyclic group containing at least one heteroatom of O, N, Si and S as a heterogeneous element, and the carbon number thereof is not particularly limited, but is preferably 2 to 60. Examples of the heterocyclic group include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group, and the arylamine group is the same as the aforementioned examples of the aryl group. In the present disclosure, the alkyl group in the aralkyl group, the alkylaryl group and the alkylamine group is the same as the aforementioned examples of the alkyl group. In the present disclosure, the heteroaryl in the heteroarylamine can apply the aforementioned description of the heterocyclic group. In the present disclosure, the alkenyl group in the aralkenyl group is the same as the aforementioned examples of the alkenyl group. In the present disclosure, the aforementioned description of the aryl group may be applied except that the arylene is a divalent group. In the present disclosure, the aforementioned description of the heterocyclic group can be applied except that the heteroarylene is a divalent group. In the present disclosure, the aforementioned description of the aryl group or cycloalkyl group can be applied except that the hydrocarbon ring is not a monovalent group but formed by combining two substituent groups. In the present disclosure, the aforementioned description of the heterocyclic group can be applied, except that the heterocycle is not a monovalent group but formed by combining two substituent groups.

In the Chemical Formula 1, at least one hydrogen may be substituted with deuterium.

Preferably, Ar is substituted or unsubstituted C₆₋₁₂ aryl. More preferably, Ar is phenyl, biphenyl, or naphthyl.

Preferably, L is a single bond, or substituted or unsubstituted C₆₋₁₂ arylene. More preferably, L is a single bond, phenylene, biphenyldiyl, terphenyldiyl, naphthylene, or -(phenylene)-(naphthylene)-. More preferably, L is a single bond, 1,4-phenylene, 4,4′-biphenyldiyl, or 2,6-naphthylene.

Preferably, L₁ and L₂ are each independently a single bond, or substituted or unsubstituted C₆₋₁₂ arylene. More preferably, L₁ and L₂ are each independently a single bond, phenylene, or biphenyldiyl. More preferably, L₁ and L₂ are each independently a single bond, 1,4-phenylene, or 4,4′-biphenyldiyl.

Preferably, Ar₁ and Ar₂ are each independently phenyl, biphenylyl, terphenylyl, naphthyl, naphthylphenyl, phenylnaphthyl, phenanthrenyl, dimethylfluorenyl, diphenylfluorenyl, dibenzofuranyl, dibenzothiophenyl, 9H-carbazol-9-yl, or 9-phenyl-9H-carbazolyl.

Preferably, one of R₁ to R₄ is the Chemical Formula 3, and the rest are each independently hydrogen or deuterium; and R₅ and R₆ are each independently hydrogen or deuterium.

Preferably, R₁ to R₄ are each independently hydrogen, or deuterium; and one of R₅ and R₆ is the Chemical Formula 3, and the rest is hydrogen or deuterium. Herein, preferably, L₁ is phenylene or biphenyldiyl, and Ar is biphenylyl, terphenylyl, naphthyl, phenanthrenyl, dimethylfluorenyl, or diphenylfluorenyl; or L₁ is a single bond, phenylene, or biphenyldiyl, and Ar is dibenzofuranyl, dibenzothiophenyl, 9H-carbazol-9-yl, or 9-phenyl-9H-carbazolyl. More preferably, Ar₁ and Ar₂ are each independently terphenylyl, naphthyl, phenanthrenyl, dimethylfluorenyl, diphenylfluorenyl, dibenzofuranyl, dibenzothiophenyl, 9H-carbazol-9-yl, or 9-phenyl-9H-carbazolyl. Alternatively, preferably, Ar is phenyl and Ar₂ is phenyl, biphenyl, terphenylyl, naphthyl, phenanthrenyl, dibenzofuranyl, dibenzothiophenyl, 9H-carbazol-9-yl, or 9-phenyl-9H-carbazolyl; or Ar is biphenylyl, and Ar₂ is terphenylyl, phenanthrenyl, dibenzofuranyl, dibenzothiophenyl, 9H-carbazol-9-yl, or 9-phenyl-9H-carbazolyl. Herein, preferably, L₁ and L₂ are each independently a single bond, phenylene, or biphenyldiyl, and more preferably, L₁ and L₂ are each independently a single bond, 1,4-phenylene, or 4,4′-biphenyldiyl.

Representative examples of the compound represented by the Chemical Formula 1 or 2 areas follows:

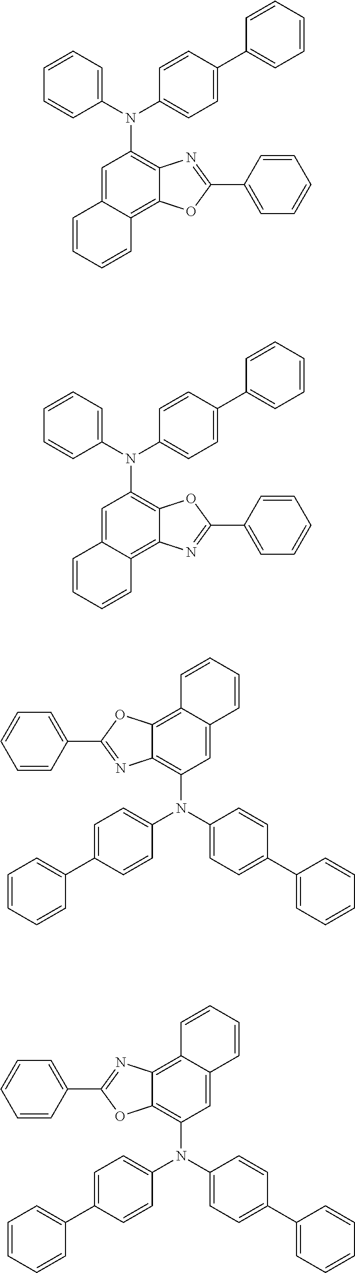 [SMILES: c1ccc(-c2ccc(N(c3ccccc3)c3cc4ccccc4c4oc(-c5ccccc5)nc34)cc2)cc1 ; c1ccc(-c2ccc(N(c3ccccc3)c3cc4ccccc4c4nc(-c5ccccc5)oc34)cc2)cc1 ; c1ccc(-c2ccc(N(c3ccc(-c4ccccc4)cc3)c3cc4ccccc4c4oc(-c5ccccc5)nc34)cc2)cc1 ; c1ccc(-c2ccc(N(c3ccc(-c4ccccc4)cc3)c3cc4ccccc4c4nc(-c5ccccc5)oc34)cc2)cc1]

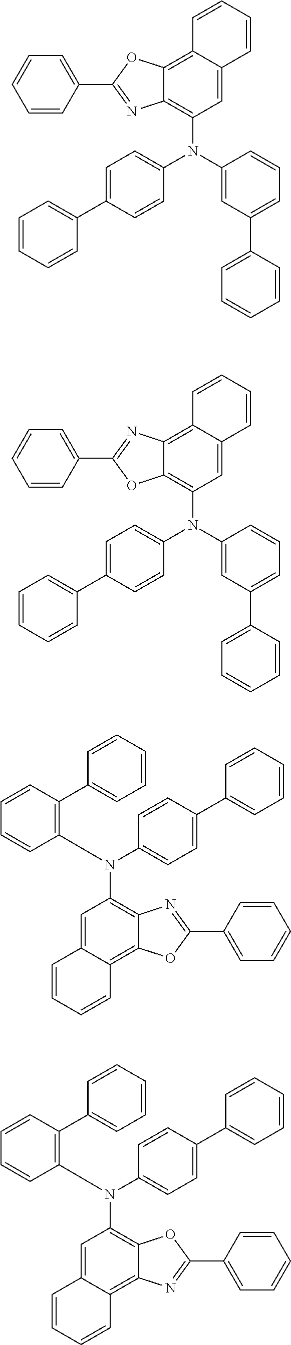 [SMILES: c1ccc(-c2ccc(N(c3cccc(-c4ccccc4)c3)c3cc4ccccc4c4oc(-c5ccccc5)nc34)cc2)cc1 ; c1ccc(-c2ccc(N(c3cccc(-c4ccccc4)c3)c3cc4ccccc4c4nc(-c5ccccc5)oc34)cc2)cc1 ; c1ccc(-c2ccc(N(c3ccccc3-c3ccccc3)c3cc4ccccc4c4oc(-c5ccccc5)nc34)cc2)cc1 ; c1ccc(-c2ccc(N(c3ccccc3-c3ccccc3)c3cc4ccccc4c4nc(-c5ccccc5)oc34)cc2)cc1]

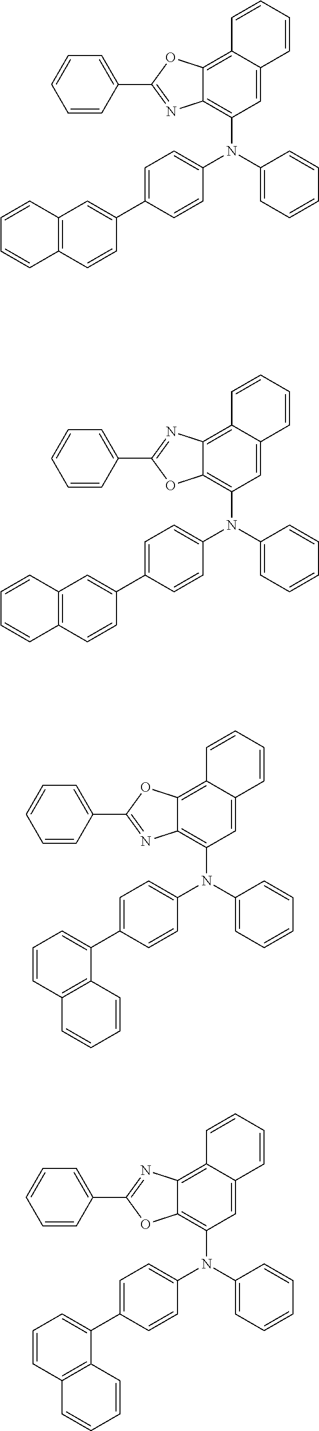 [SMILES: c1ccc(-c2nc3c(N(c4ccccc4)c4ccc(-c5ccc6ccccc6c5)cc4)cc4ccccc4c3o2)cc1 ; c1ccc(-c2nc3c(o2)c(N(c2ccccc2)c2ccc(-c4ccc5ccccc5c4)cc2)cc2ccccc23)cc1 ; c1ccc(-c2nc3c(N(c4ccccc4)c4ccc(-c5cccc6ccccc56)cc4)cc4ccccc4c3o2)cc1 ; c1ccc(-c2nc3c(o2)c(N(c2ccccc2)c2ccc(-c4cccc5ccccc45)cc2)cc2ccccc23)cc1]

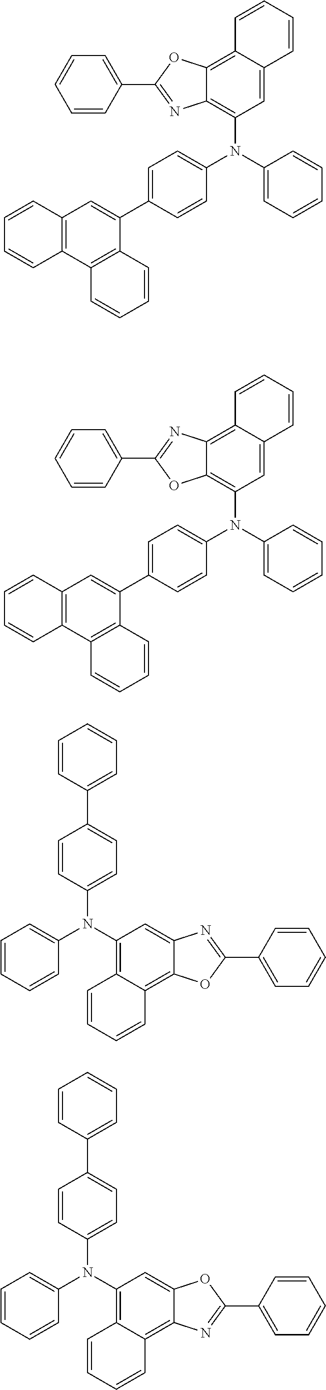

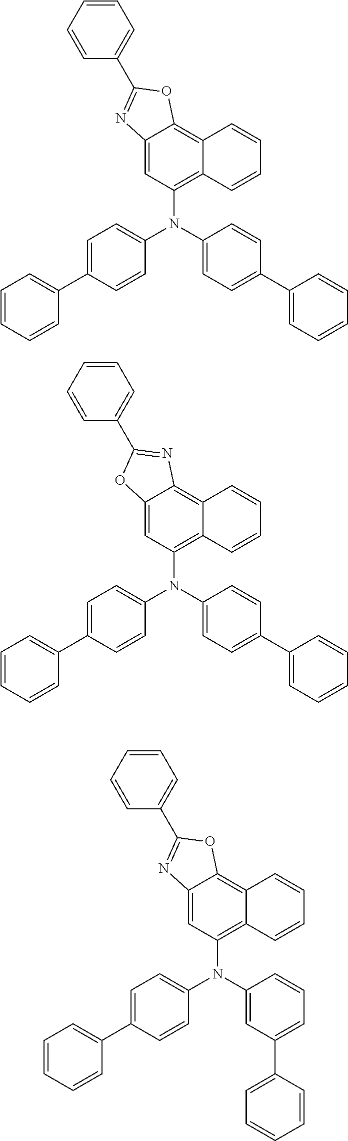

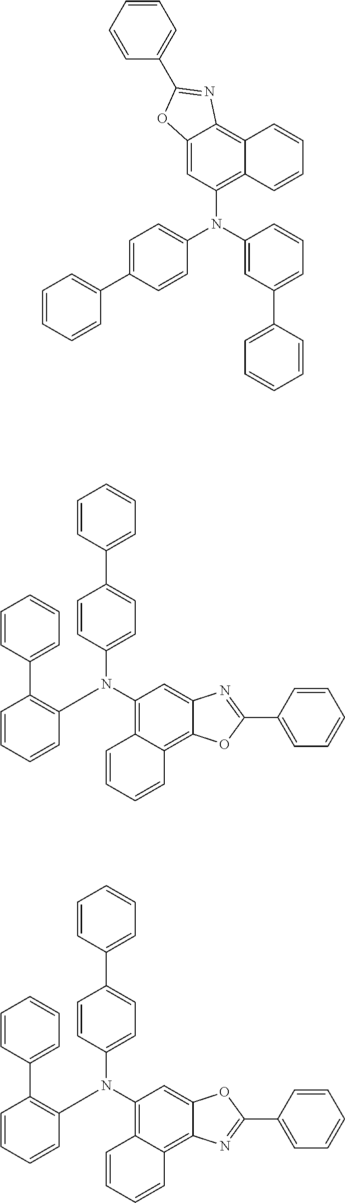 [SMILES: c1ccc(-c2ccc(N(c3cccc(-c4ccccc4)c3)c3cc4oc(-c5ccccc5)nc4c4ccccc34)cc2)cc1 ; c1ccc(-c2ccc(N(c3ccccc3-c3ccccc3)c3cc4nc(-c5ccccc5)oc4c4ccccc34)cc2)cc1 ; c1ccc(-c2ccc(N(c3ccccc3-c3ccccc3)c3cc4oc(-c5ccccc5)nc4c4ccccc34)cc2)cc1]

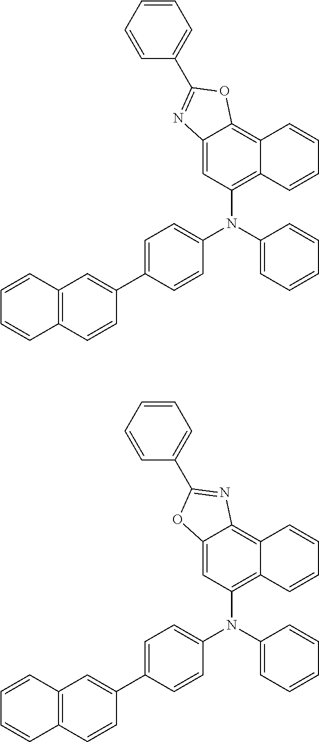 [SMILES: c1ccc(-c2nc3cc(N(c4ccccc4)c4ccc(-c5ccc6ccccc6c5)cc4)c4ccccc4c3o2)cc1 ; c1ccc(-c2nc3c(cc(N(c4ccccc4)c4ccc(-c5ccc6ccccc6c5)cc4)c4ccccc43)o2)cc1]

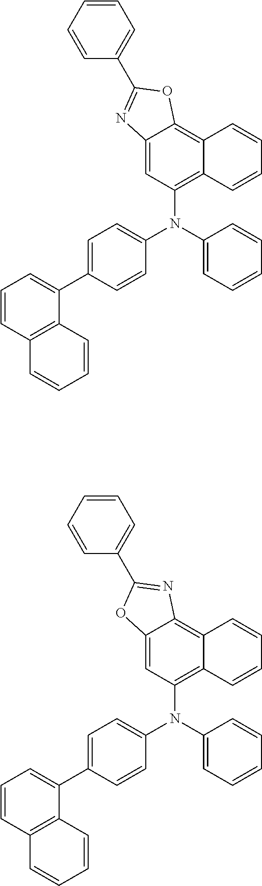 [SMILES: c1ccc(-c2nc3cc(N(c4ccccc4)c4ccc(-c5cccc6ccccc56)cc4)c4ccccc4c3o2)cc1 ; c1ccc(-c2nc3c(cc(N(c4ccccc4)c4ccc(-c5cccc6ccccc56)cc4)c4ccccc43)o2)cc1]

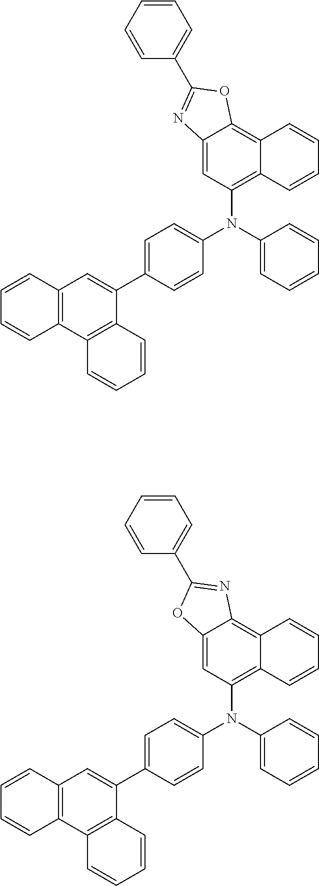 [SMILES: c1ccc(-c2nc3cc(N(c4ccccc4)c4ccc(-c5cc6ccccc6c6ccccc56)cc4)c4ccccc4c3o2)cc1 ; c1ccc(-c2nc3c(cc(N(c4ccccc4)c4ccc(-c5cc6ccccc6c6ccccc56)cc4)c4ccccc43)o2)cc1]

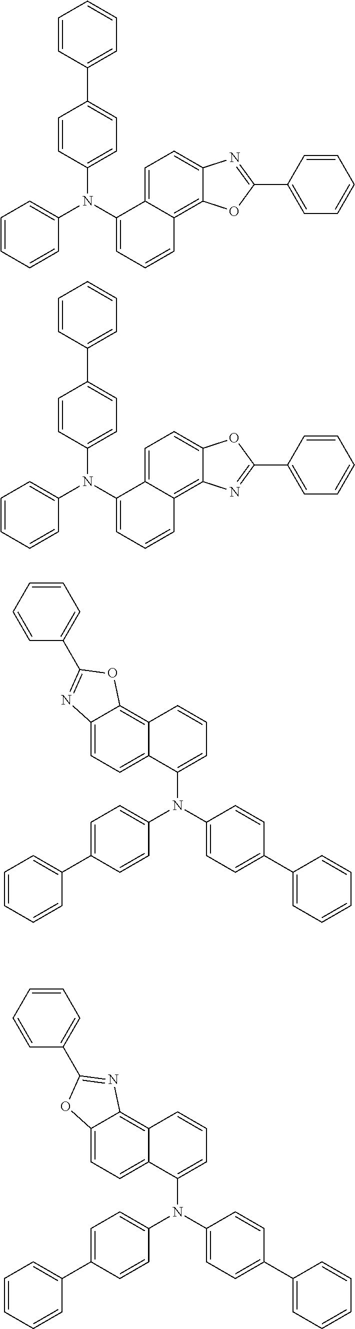

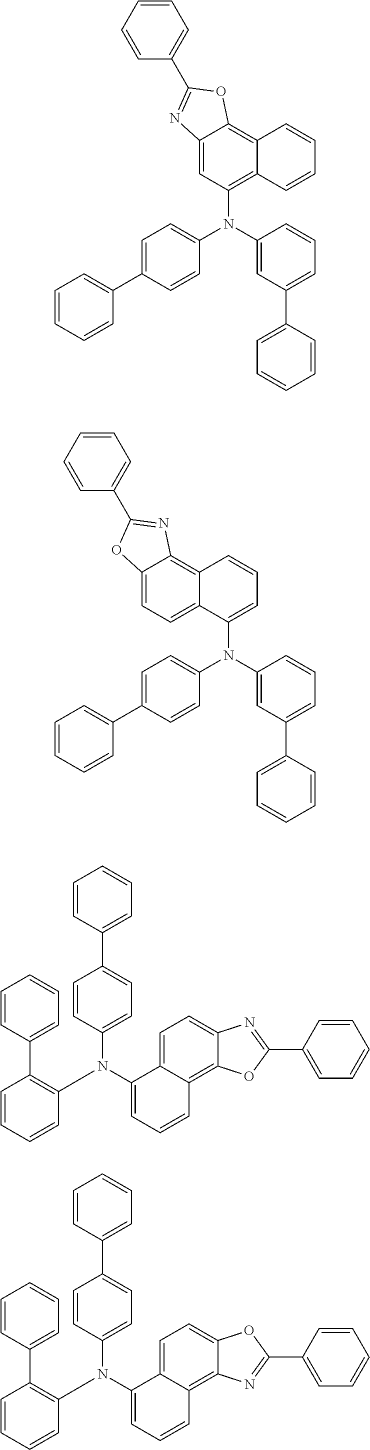 [SMILES: c1ccc(-c2ccc(N(c3cccc(-c4ccccc4)c3)c3cc4nc(-c5ccccc5)oc4c4ccccc34)cc2)cc1 ; c1ccc(-c2ccc(N(c3cccc(-c4ccccc4)c3)c3cccc4c3ccc3oc(-c5ccccc5)nc34)cc2)cc1 ; c1ccc(-c2ccc(N(c3ccccc3-c3ccccc3)c3cccc4c3ccc3nc(-c5ccccc5)oc34)cc2)cc1 ; c1ccc(-c2ccc(N(c3ccccc3-c3ccccc3)c3cccc4c3ccc3oc(-c5ccccc5)nc34)cc2)cc1]

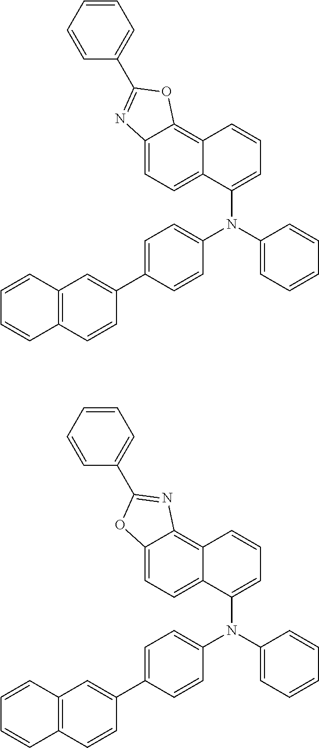 [SMILES: c1ccc(-c2nc3ccc4c(N(c5ccccc5)c5ccc(-c6ccc7ccccc7c6)cc5)cccc4c3o2)cc1 ; c1ccc(-c2nc3c(ccc4c(N(c5ccccc5)c5ccc(-c6ccc7ccccc7c6)cc5)cccc43)o2)cc1]

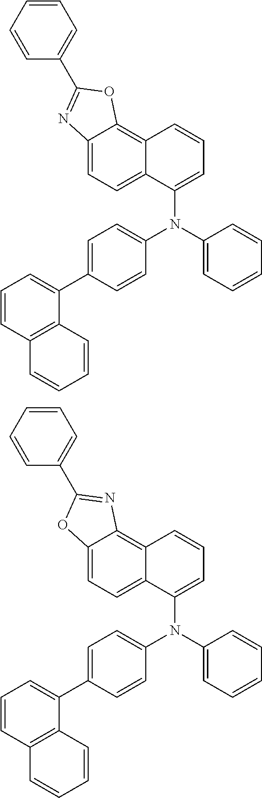

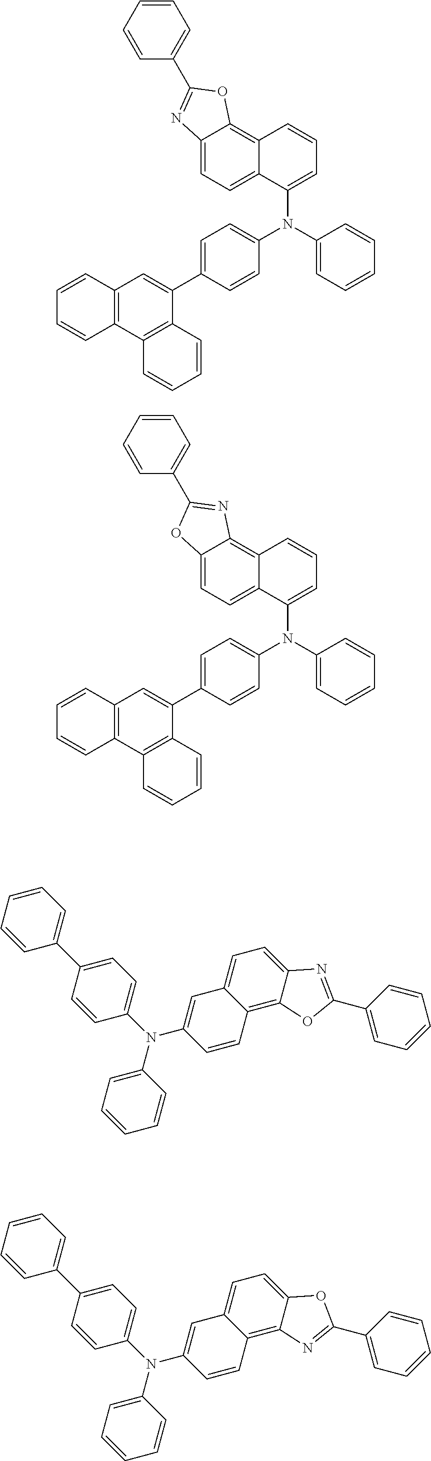

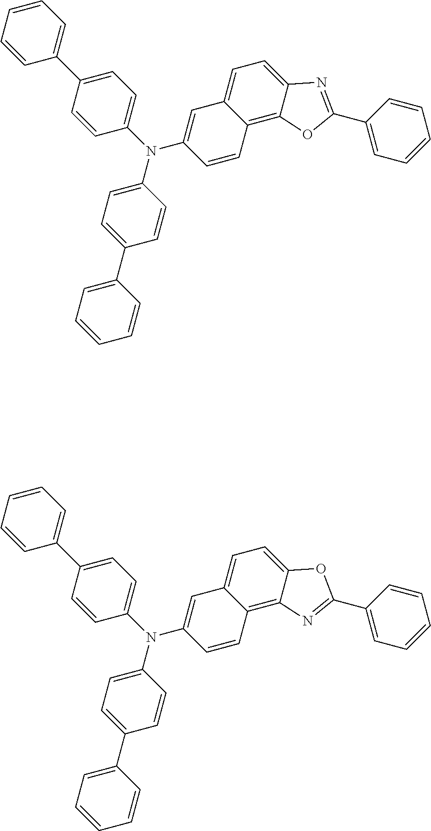 [SMILES: c1ccc(-c2ccc(N(c3ccc(-c4ccccc4)cc3)c3ccc4c(ccc5nc(-c6ccccc6)oc54)c3)cc2)cc1 ; c1ccc(-c2ccc(N(c3ccc(-c4ccccc4)cc3)c3ccc4c(ccc5oc(-c6ccccc6)nc54)c3)cc2)cc1]

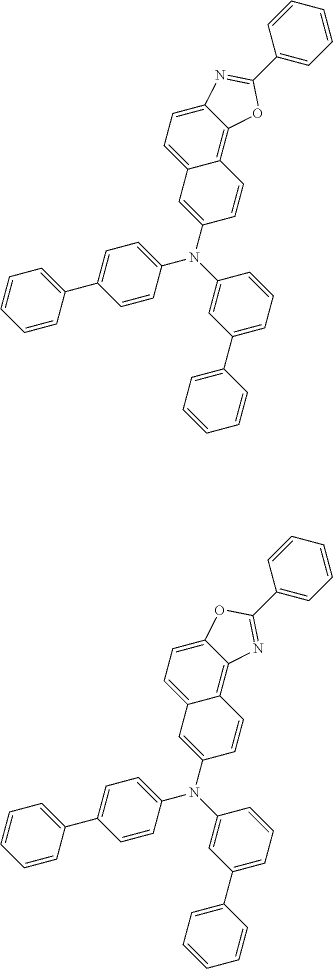 [SMILES: c1ccc(-c2ccc(N(c3cccc(-c4ccccc4)c3)c3ccc4c(ccc5nc(-c6ccccc6)oc54)c3)cc2)cc1 ; c1ccc(-c2ccc(N(c3cccc(-c4ccccc4)c3)c3ccc4c(ccc5oc(-c6ccccc6)nc54)c3)cc2)cc1]

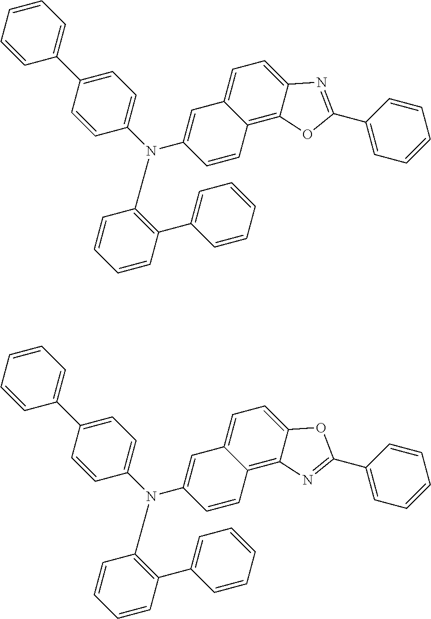 [SMILES: c1ccc(-c2ccc(N(c3ccc4c(ccc5nc(-c6ccccc6)oc54)c3)c3ccccc3-c3ccccc3)cc2)cc1 ; c1ccc(-c2ccc(N(c3ccc4c(ccc5oc(-c6ccccc6)nc54)c3)c3ccccc3-c3ccccc3)cc2)cc1]

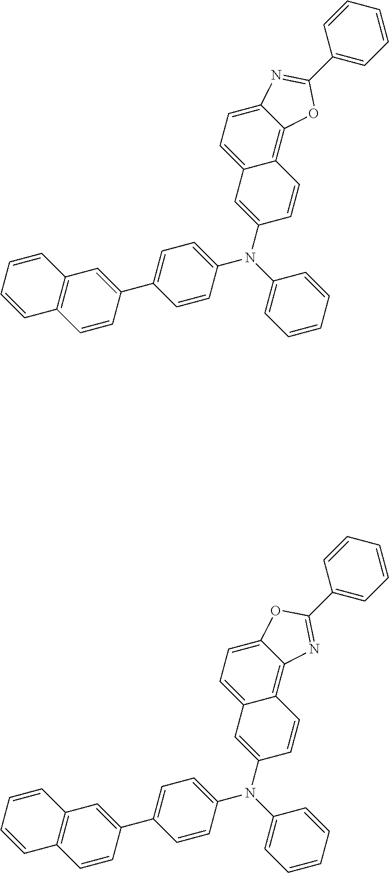 [SMILES: c1ccc(-c2nc3ccc4cc(N(c5ccccc5)c5ccc(-c6ccc7ccccc7c6)cc5)ccc4c3o2)cc1 ; c1ccc(-c2nc3c(ccc4cc(N(c5ccccc5)c5ccc(-c6ccc7ccccc7c6)cc5)ccc43)o2)cc1]

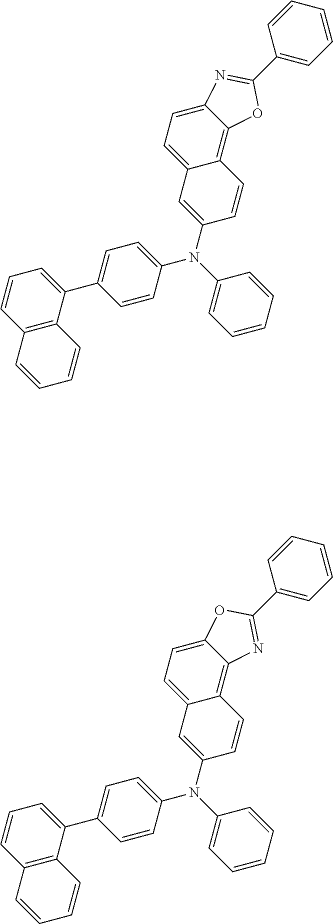 [SMILES: c1ccc(-c2nc3ccc4cc(N(c5ccccc5)c5ccc(-c6cccc7ccccc67)cc5)ccc4c3o2)cc1 ; c1ccc(-c2nc3c(ccc4cc(N(c5ccccc5)c5ccc(-c6cccc7ccccc67)cc5)ccc43)o2)cc1]

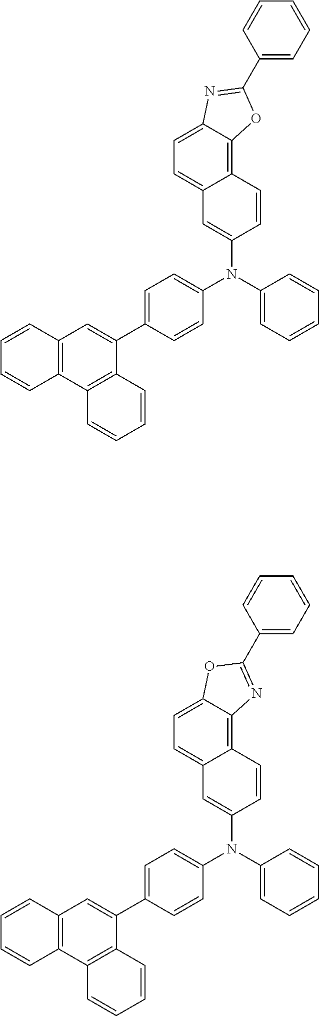 [SMILES: c1ccc(-c2nc3ccc4cc(N(c5ccccc5)c5ccc(-c6cc7ccccc7c7ccccc67)cc5)ccc4c3o2)cc1 ; c1ccc(-c2nc3c(ccc4cc(N(c5ccccc5)c5ccc(-c6cc7ccccc7c7ccccc67)cc5)ccc43)o2)cc1]

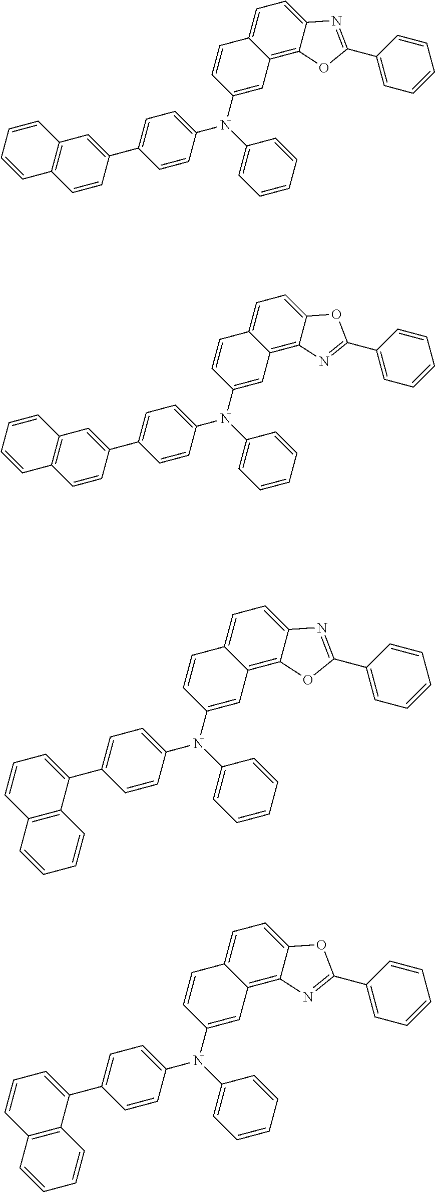 [SMILES: c1ccc(-c2nc3ccc4ccc(N(c5ccccc5)c5ccc(-c6ccc7ccccc7c6)cc5)cc4c3o2)cc1 ; c1ccc(-c2nc3c(ccc4ccc(N(c5ccccc5)c5ccc(-c6ccc7ccccc7c6)cc5)cc43)o2)cc1 ; c1ccc(-c2nc3ccc4ccc(N(c5ccccc5)c5ccc(-c6cccc7ccccc67)cc5)cc4c3o2)cc1 ; c1ccc(-c2nc3c(ccc4ccc(N(c5ccccc5)c5ccc(-c6cccc7ccccc67)cc5)cc43)o2)cc1]

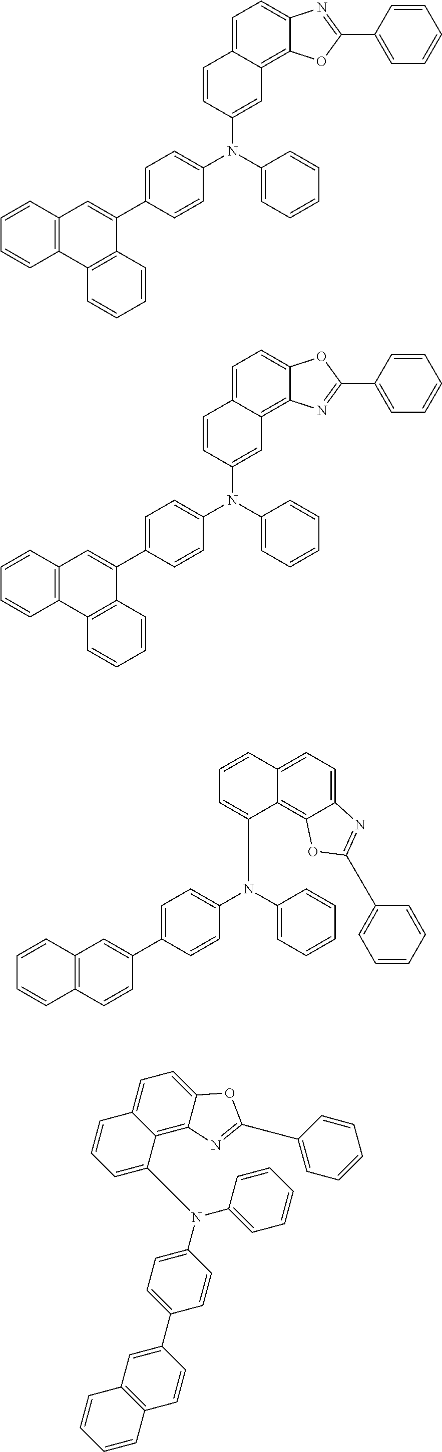 [SMILES: c1ccc(-c2nc3ccc4ccc(N(c5ccccc5)c5ccc(-c6cc7ccccc7c7ccccc67)cc5)cc4c3o2)cc1 ; c1ccc(-c2nc3c(ccc4ccc(N(c5ccccc5)c5ccc(-c6cc7ccccc7c7ccccc67)cc5)cc43)o2)cc1 ; c1ccc(-c2nc3ccc4cccc(N(c5ccccc5)c5ccc(-c6ccc7ccccc7c6)cc5)c4c3o2)cc1 ; c1ccc(-c2nc3c(ccc4cccc(N(c5ccccc5)c5ccc(-c6ccc7ccccc7c6)cc5)c43)o2)cc1]

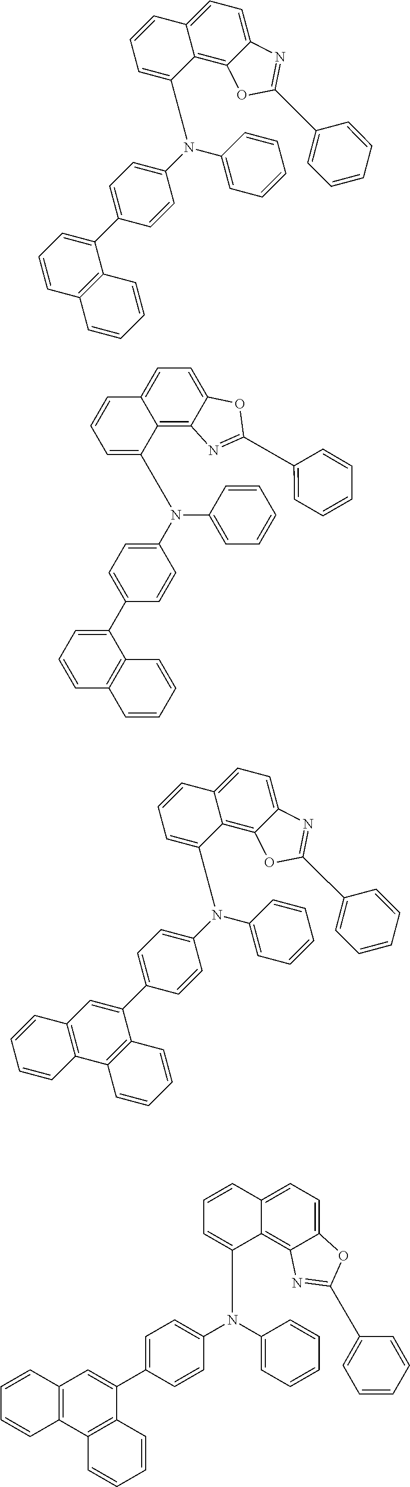 [SMILES: c1ccc(-c2nc3ccc4cccc(N(c5ccccc5)c5ccc(-c6cccc7ccccc67)cc5)c4c3o2)cc1 ; c1ccc(-c2nc3c(ccc4cccc(N(c5ccccc5)c5ccc(-c6cccc7ccccc67)cc5)c43)o2)cc1 ; c1ccc(-c2nc3ccc4cccc(N(c5ccccc5)c5ccc(-c6cc7ccccc7c7ccccc67)cc5)c4c3o2)cc1 ; c1ccc(-c2nc3c(ccc4cccc(N(c5ccccc5)c5ccc(-c6cc7ccccc7c7ccccc67)cc5)c43)o2)cc1]

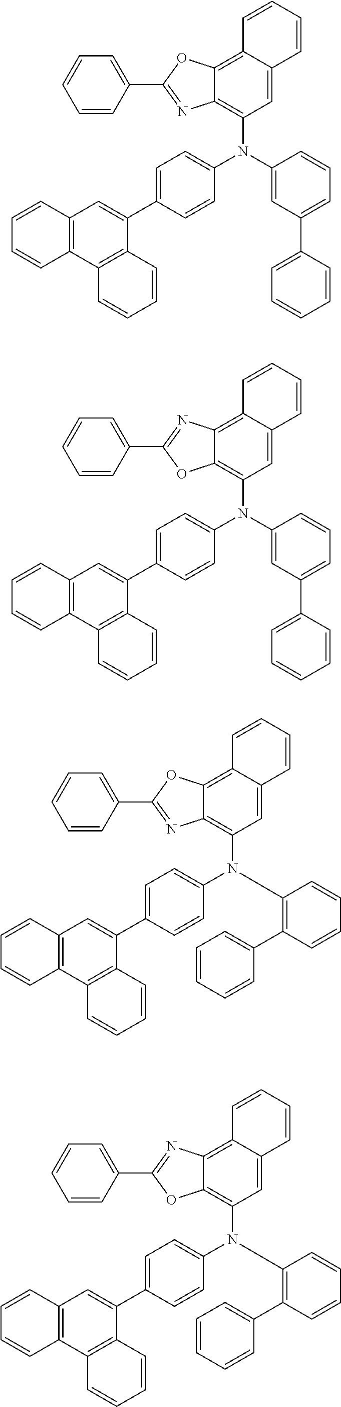 [SMILES: c1ccc(-c2cccc(N(c3ccc(-c4cc5ccccc5c5ccccc45)cc3)c3cc4ccccc4c4oc(-c5ccccc5)nc34)c2)cc1 ; c1ccc(-c2cccc(N(c3ccc(-c4cc5ccccc5c5ccccc45)cc3)c3cc4ccccc4c4nc(-c5ccccc5)oc34)c2)cc1 ; c1ccc(-c2nc3c(N(c4ccc(-c5cc6ccccc6c6ccccc56)cc4)c4ccccc4-c4ccccc4)cc4ccccc4c3o2)cc1 ; c1ccc(-c2nc3c(o2)c(N(c2ccc(-c4cc5ccccc5c5ccccc45)cc2)c2ccccc2-c2ccccc2)cc2ccccc23)cc1]

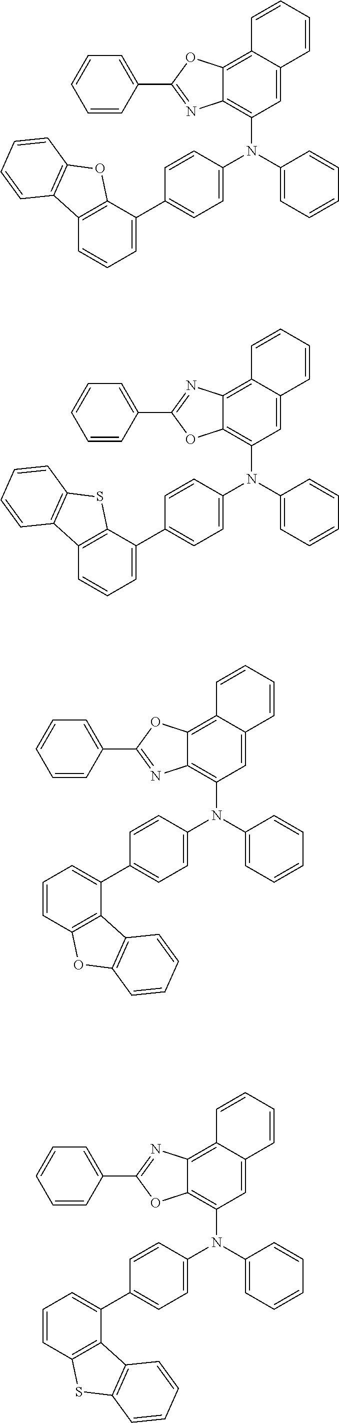 [SMILES: c1ccc(-c2nc3c(N(c4ccccc4)c4ccc(-c5cccc6c5oc5ccccc56)cc4)cc4ccccc4c3o2)cc1 ; c1ccc(-c2nc3c(o2)c(N(c2ccccc2)c2ccc(-c4cccc5c4sc4ccccc45)cc2)cc2ccccc23)cc1 ; c1ccc(-c2nc3c(N(c4ccccc4)c4ccc(-c5cccc6oc7ccccc7c56)cc4)cc4ccccc4c3o2)cc1 ; c1ccc(-c2nc3c(o2)c(N(c2ccccc2)c2ccc(-c4cccc5sc6ccccc6c45)cc2)cc2ccccc23)cc1]

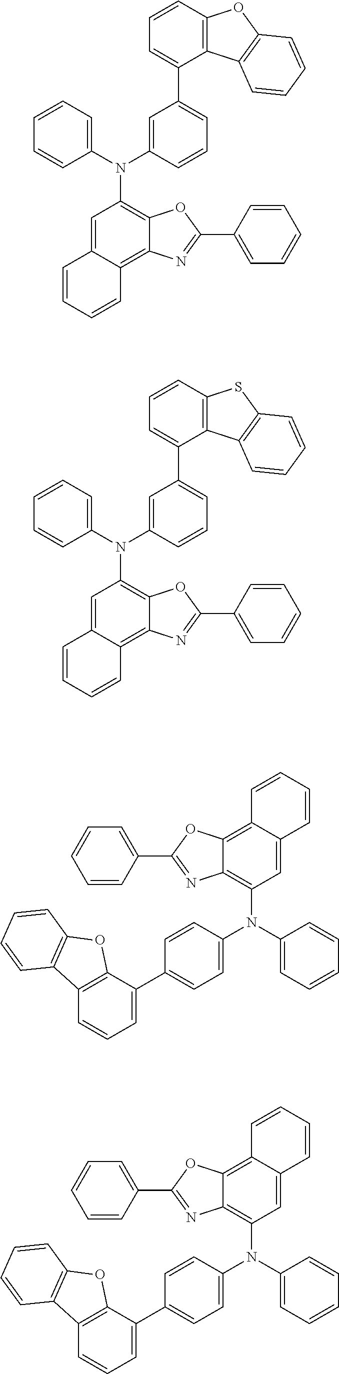 [SMILES: c1ccc(-c2nc3c(o2)c(N(c2ccccc2)c2cccc(-c4cccc5oc6ccccc6c45)c2)cc2ccccc23)cc1 ; c1ccc(-c2nc3c(o2)c(N(c2ccccc2)c2cccc(-c4cccc5sc6ccccc6c45)c2)cc2ccccc23)cc1 ; c1ccc(-c2nc3c(N(c4ccccc4)c4ccc(-c5cccc6c5oc5ccccc56)cc4)cc4ccccc4c3o2)cc1 ; c1ccc(-c2nc3c(N(c4ccccc4)c4ccc(-c5cccc6c5oc5ccccc56)cc4)cc4ccccc4c3o2)cc1]

[chemical structure]

[chemical structure]

[chemical structure]

[chemical structure]

[chemical structure]

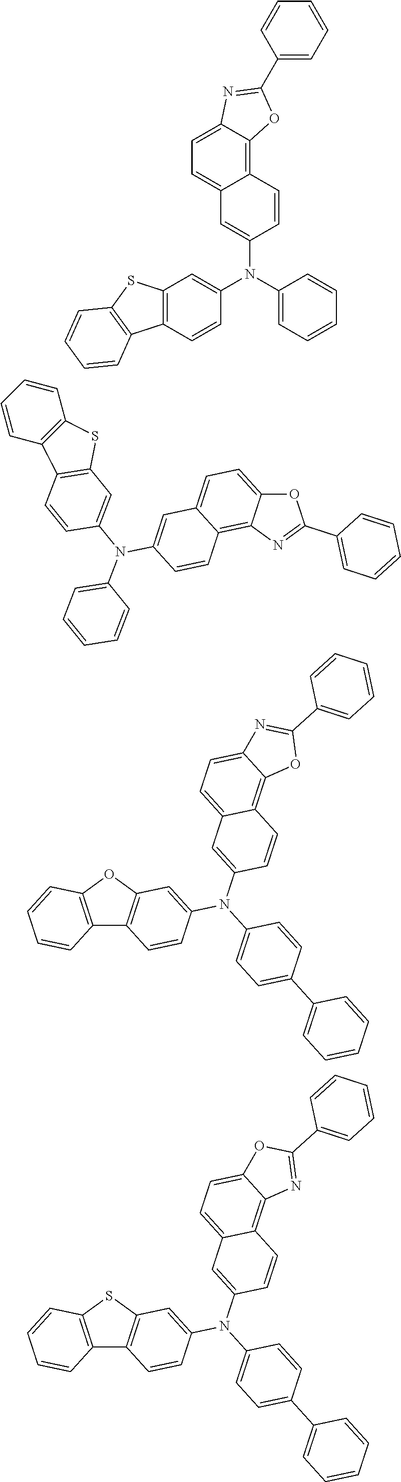

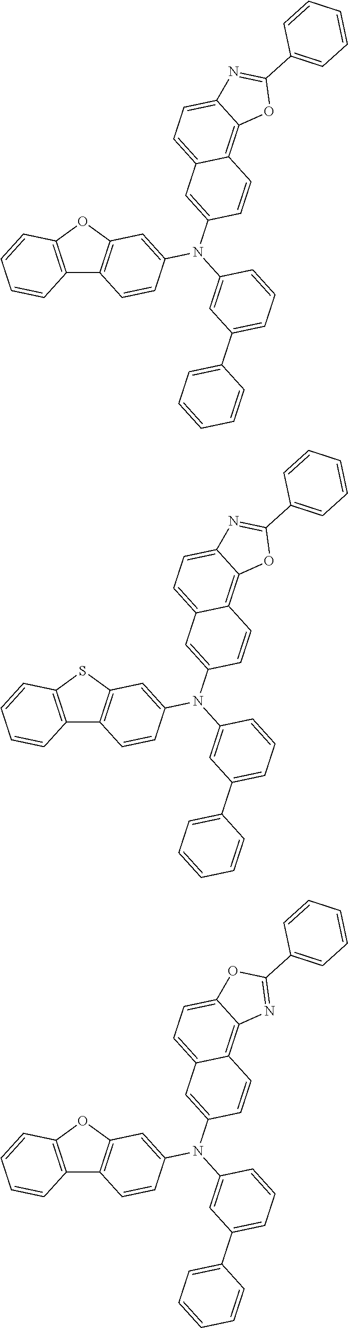 [SMILES: c1ccc(-c2cccc(N(c3ccc4c(ccc5nc(-c6ccccc6)oc54)c3)c3ccc4c(c3)oc3ccccc34)c2)cc1 ; c1ccc(-c2cccc(N(c3ccc4c(ccc5nc(-c6ccccc6)oc54)c3)c3ccc4c(c3)sc3ccccc34)c2)cc1 ; c1ccc(-c2cccc(N(c3ccc4c(ccc5oc(-c6ccccc6)nc54)c3)c3ccc4c(c3)oc3ccccc34)c2)cc1]

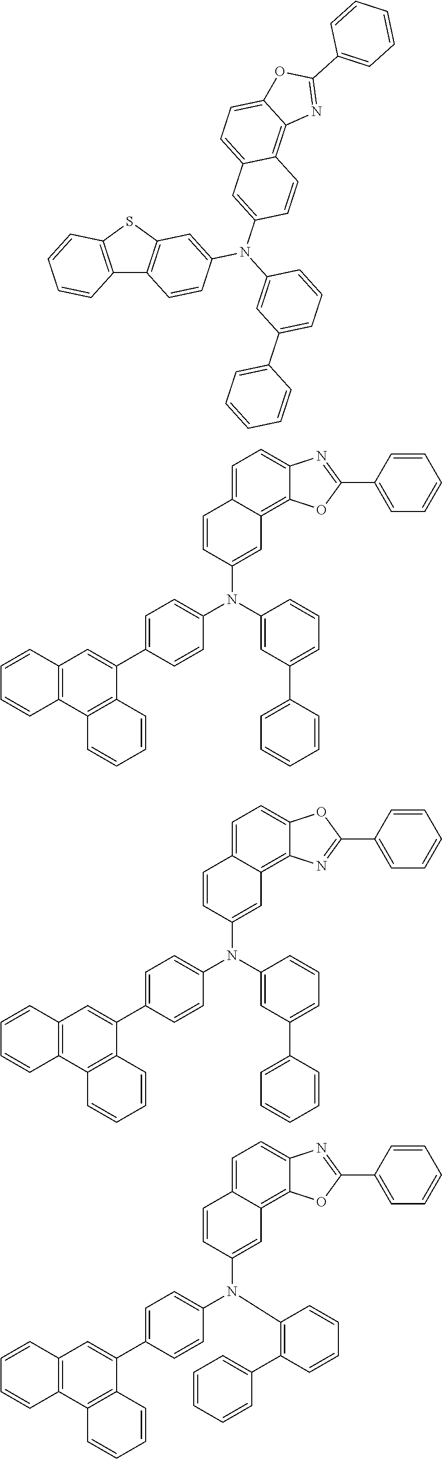

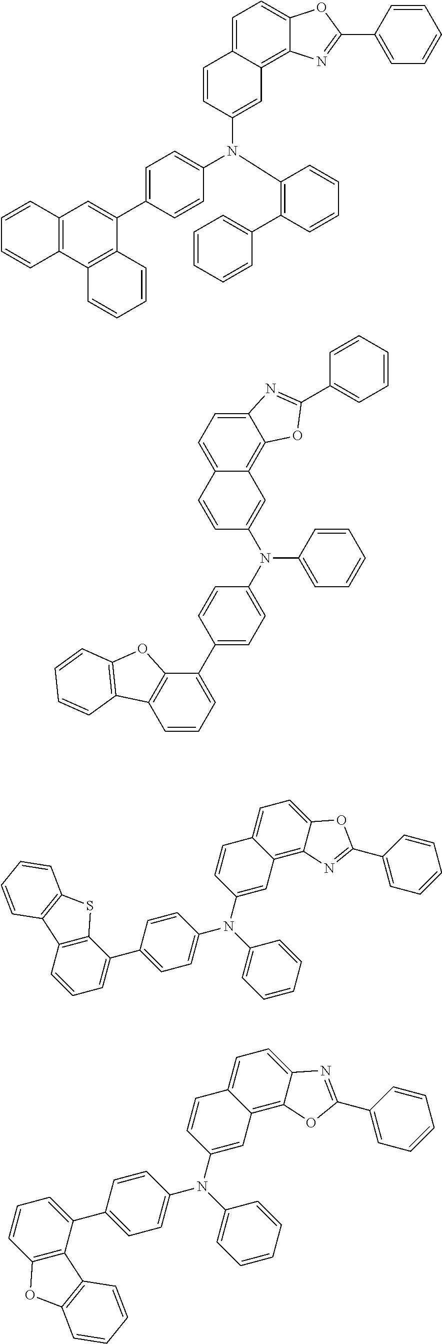 [SMILES: c1ccc(-c2nc3c(ccc4ccc(N(c5ccc(-c6cc7ccccc7c7ccccc67)cc5)c5ccccc5-c5ccccc5)cc43)o2)cc1 ; c1ccc(-c2nc3ccc4ccc(N(c5ccccc5)c5ccc(-c6cccc7c6oc6ccccc67)cc5)cc4c3o2)cc1 ; c1ccc(-c2nc3c(ccc4ccc(N(c5ccccc5)c5ccc(-c6cccc7c6sc6ccccc67)cc5)cc43)o2)cc1 ; c1ccc(-c2nc3ccc4ccc(N(c5ccccc5)c5ccc(-c6cccc7oc8ccccc8c67)cc5)cc4c3o2)cc1]

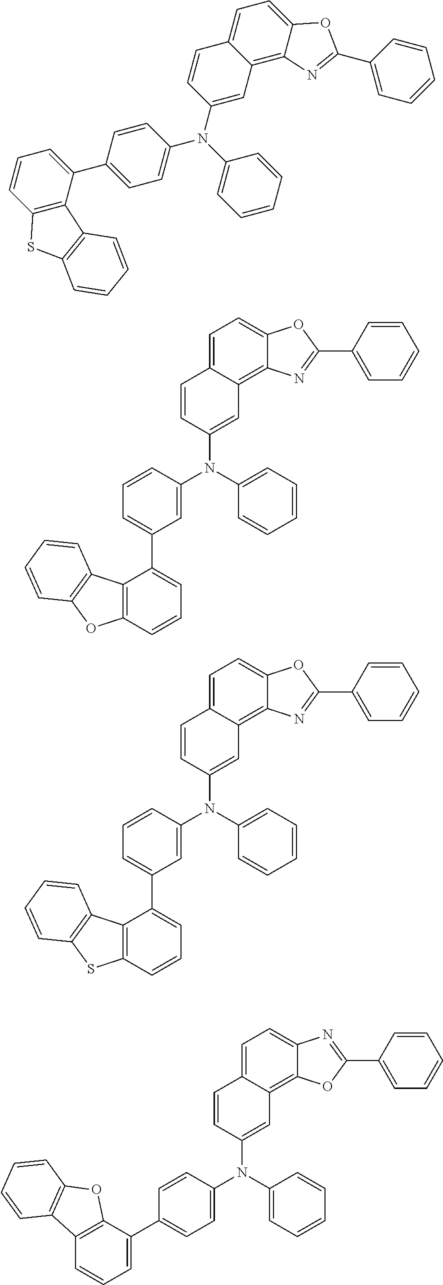 [SMILES: c1ccc(-c2nc3c(ccc4ccc(N(c5ccccc5)c5ccc(-c6cccc7sc8ccccc8c67)cc5)cc43)o2)cc1 ; c1ccc(-c2nc3c(ccc4ccc(N(c5ccccc5)c5cccc(-c6cccc7oc8ccccc8c67)c5)cc43)o2)cc1 ; c1ccc(-c2nc3c(ccc4ccc(N(c5ccccc5)c5cccc(-c6cccc7sc8ccccc8c67)c5)cc43)o2)cc1 ; c1ccc(-c2nc3ccc4ccc(N(c5ccccc5)c5ccc(-c6cccc7c6oc6ccccc67)cc5)cc4c3o2)cc1]

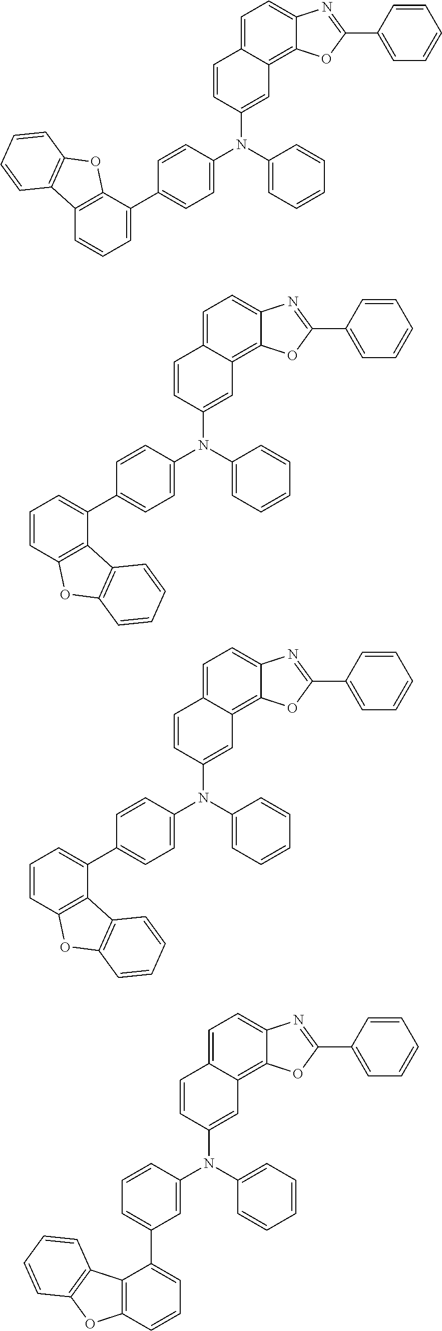 [SMILES: c1ccc(-c2nc3ccc4ccc(N(c5ccccc5)c5ccc(-c6cccc7c6oc6ccccc67)cc5)cc4c3o2)cc1 ; c1ccc(-c2nc3ccc4ccc(N(c5ccccc5)c5ccc(-c6cccc7oc8ccccc8c67)cc5)cc4c3o2)cc1 ; c1ccc(-c2nc3ccc4ccc(N(c5ccccc5)c5ccc(-c6cccc7oc8ccccc8c67)cc5)cc4c3o2)cc1 ; c1ccc(-c2nc3ccc4ccc(N(c5ccccc5)c5cccc(-c6cccc7oc8ccccc8c67)c5)cc4c3o2)cc1]

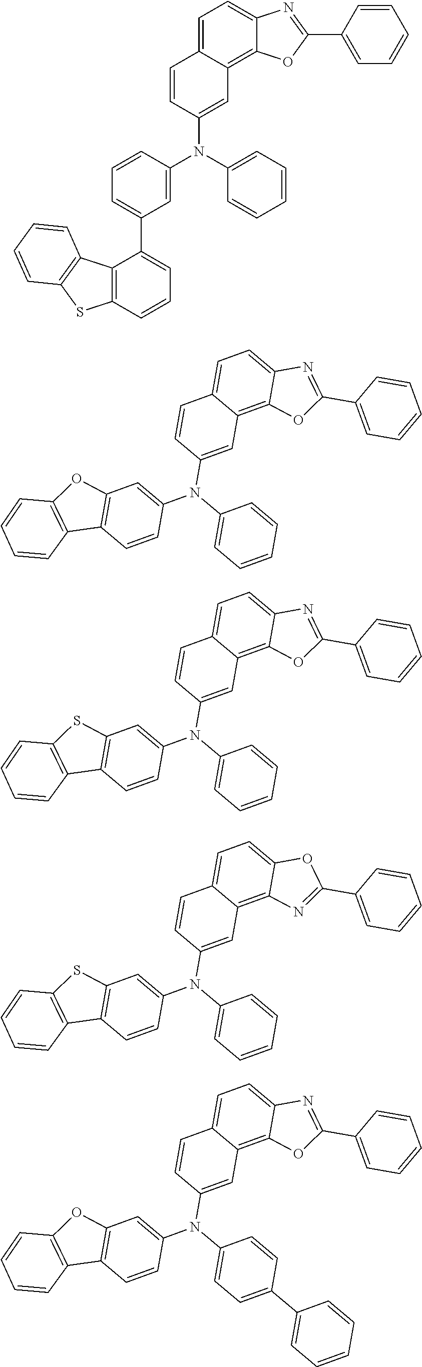

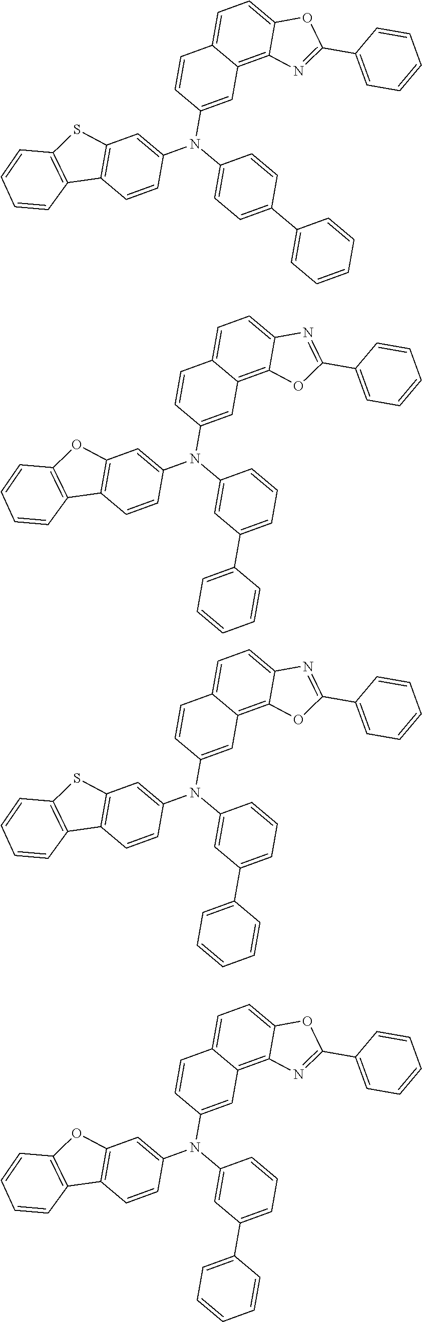

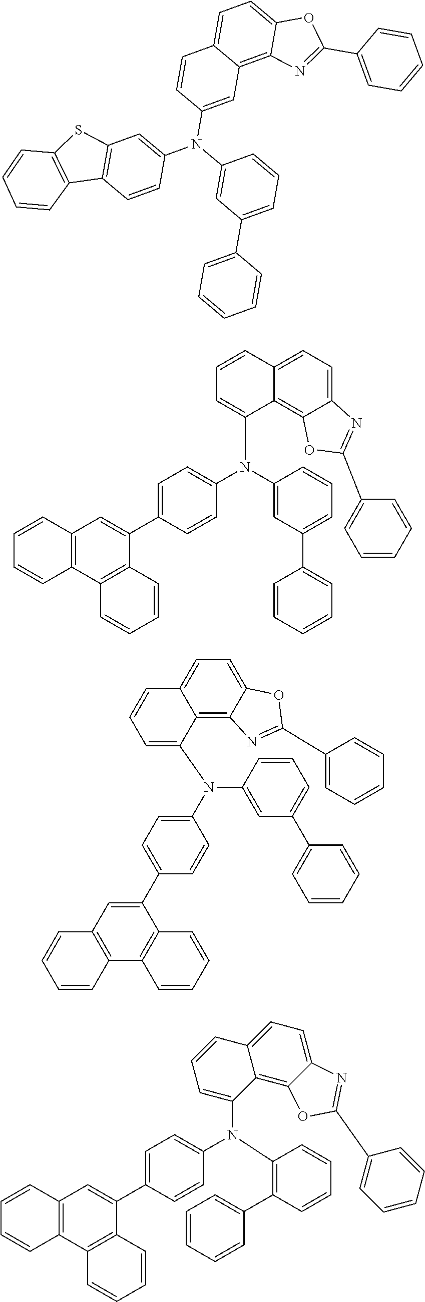

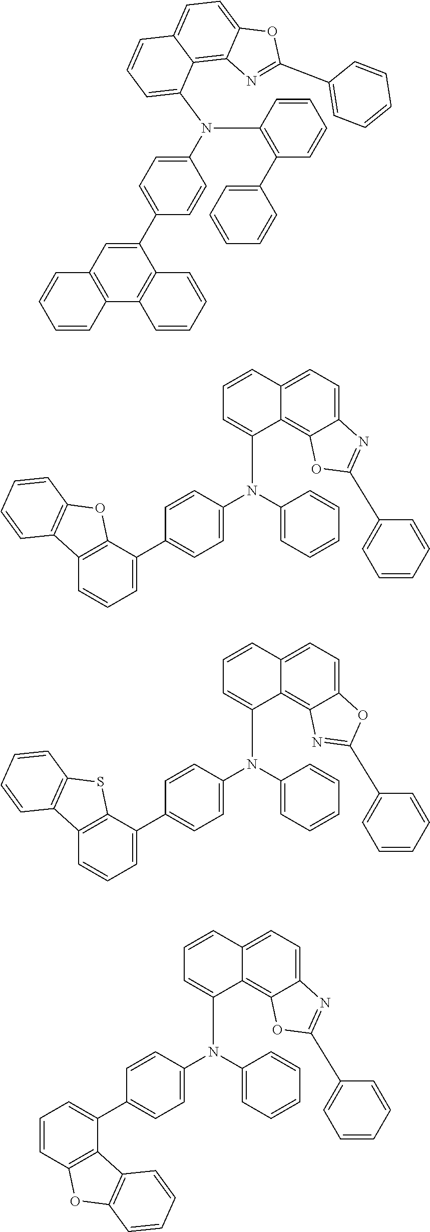 [SMILES: c1ccc(-c2nc3c(ccc4cccc(N(c5ccc(-c6cc7ccccc7c7ccccc67)cc5)c5ccccc5-c5ccccc5)c43)o2)cc1 ; c1ccc(-c2nc3ccc4cccc(N(c5ccccc5)c5ccc(-c6cccc7c6oc6ccccc67)cc5)c4c3o2)cc1 ; c1ccc(-c2nc3c(ccc4cccc(N(c5ccccc5)c5ccc(-c6cccc7c6sc6ccccc67)cc5)c43)o2)cc1 ; c1ccc(-c2nc3ccc4cccc(N(c5ccccc5)c5ccc(-c6cccc7oc8ccccc8c67)cc5)c4c3o2)cc1]

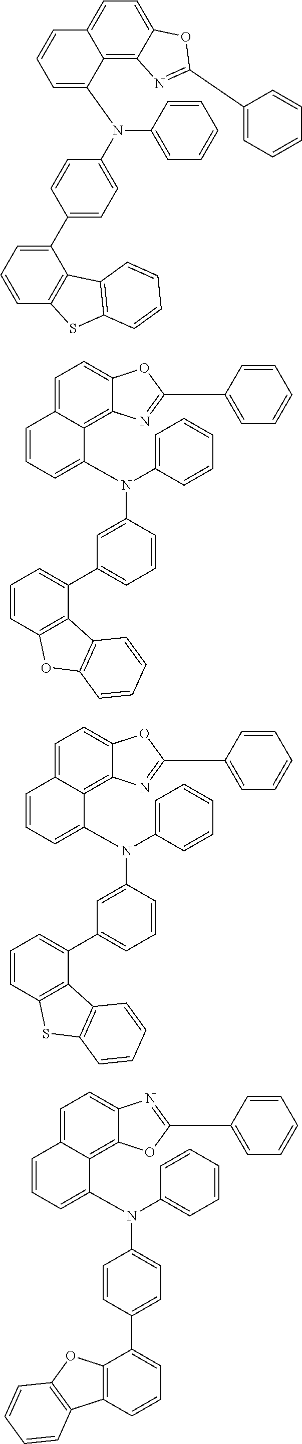

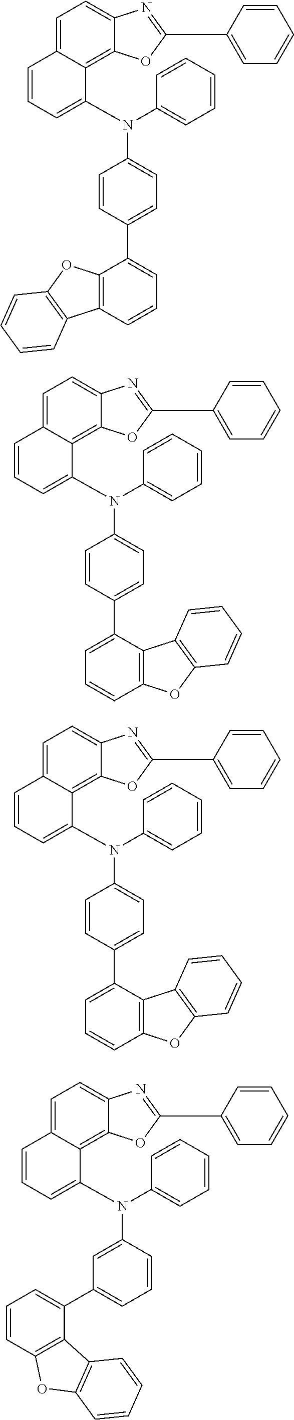

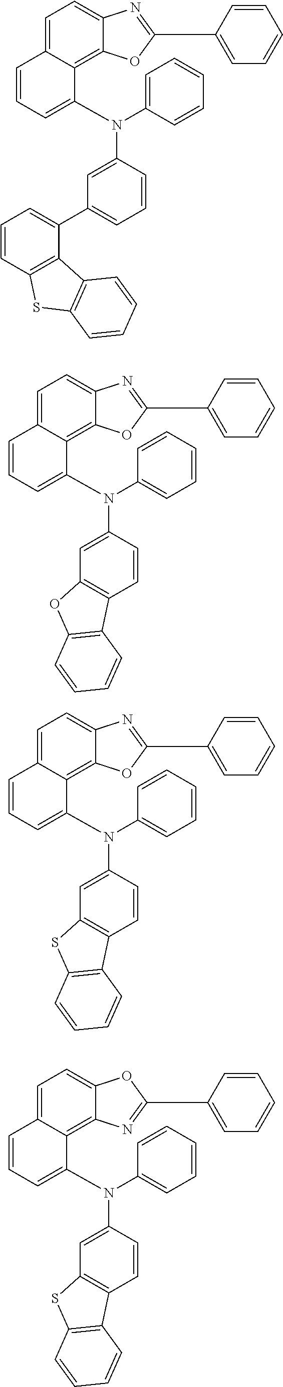

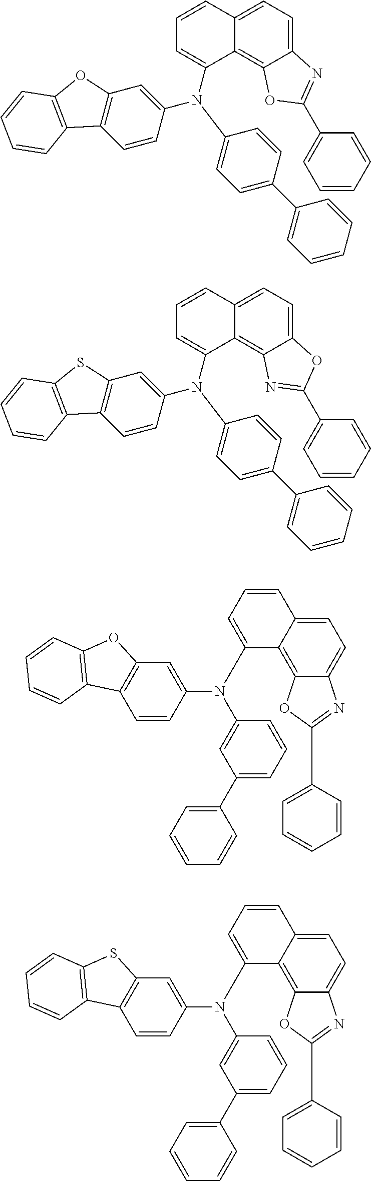

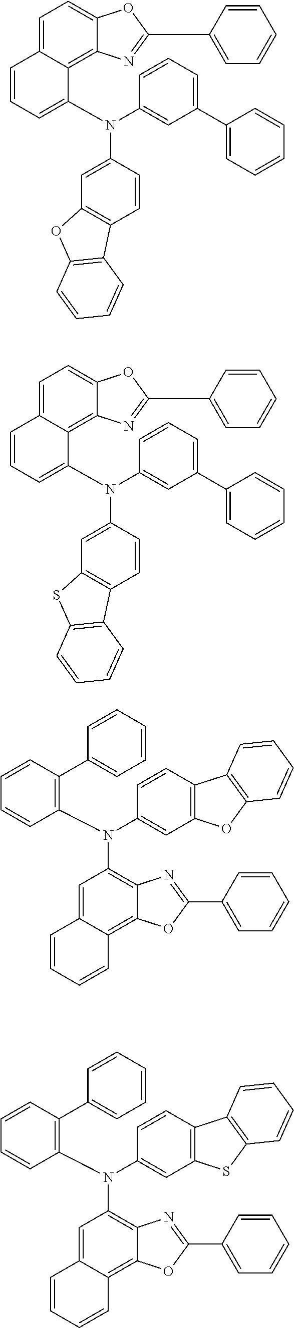

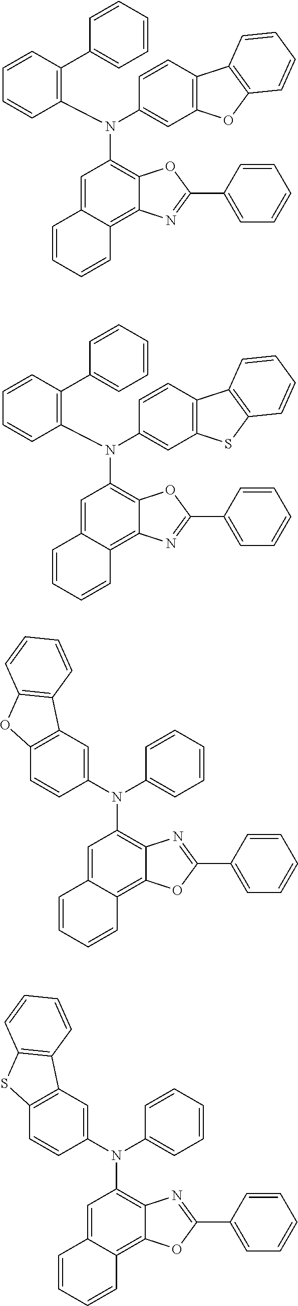

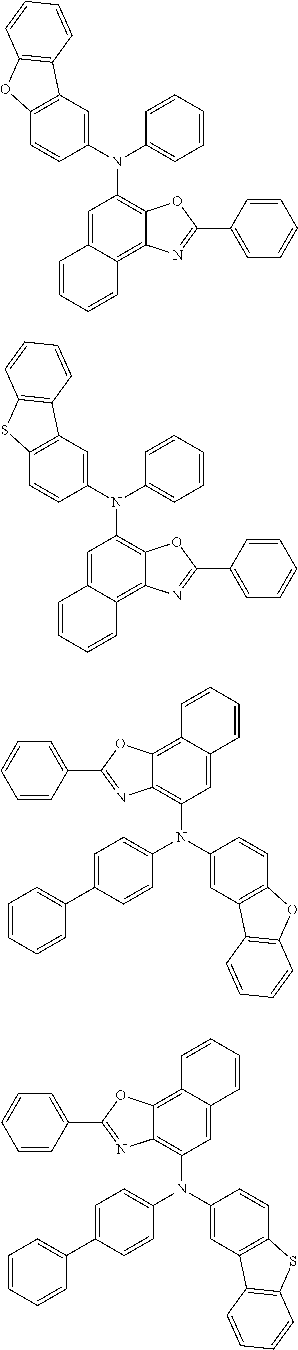

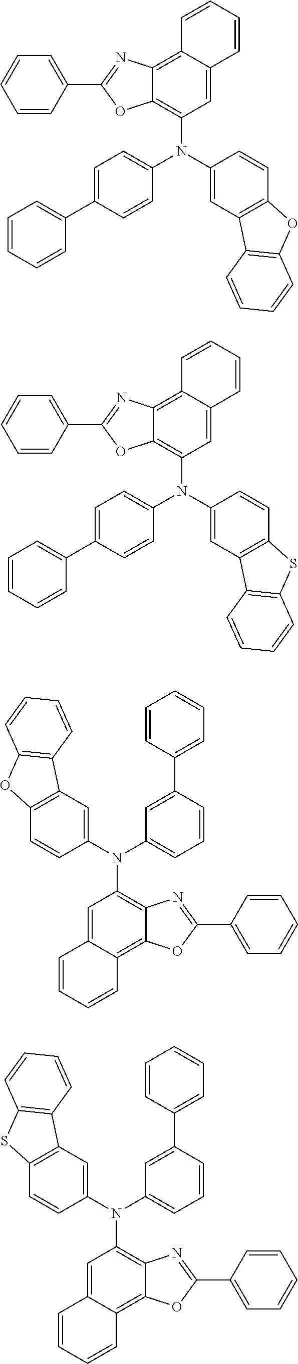

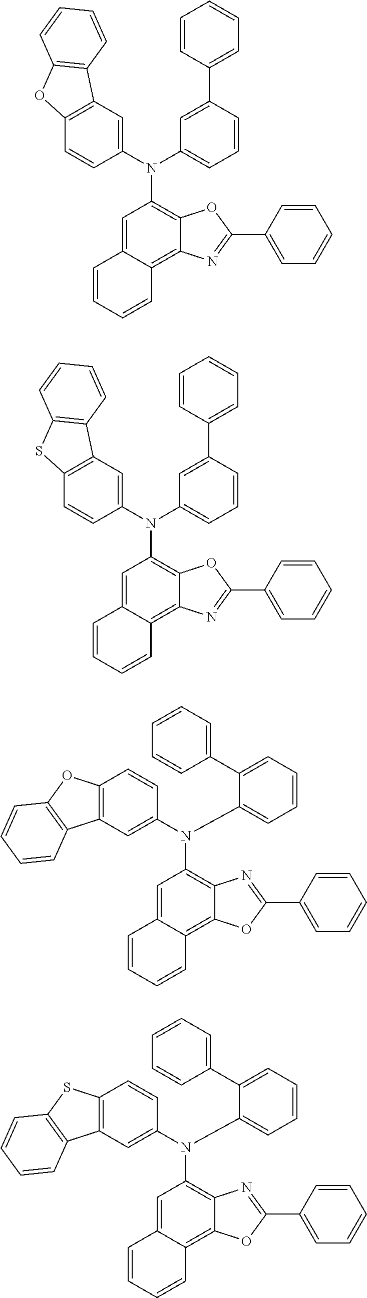

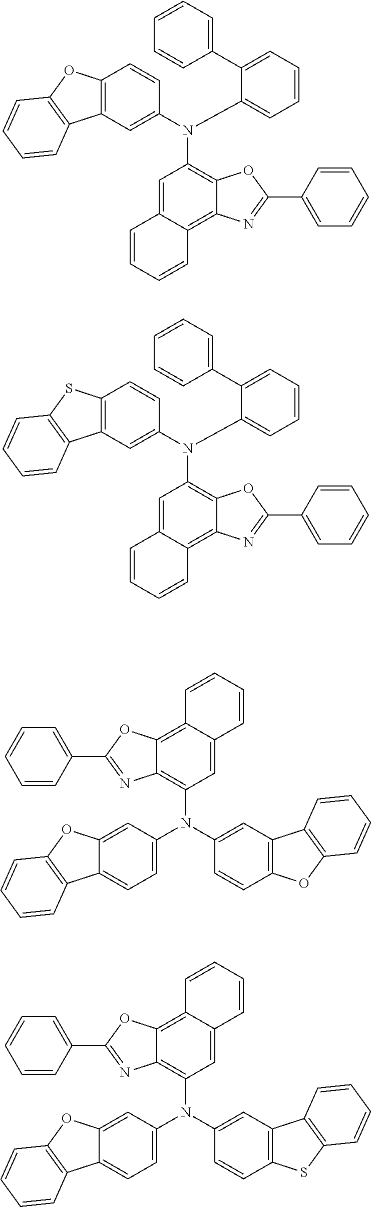

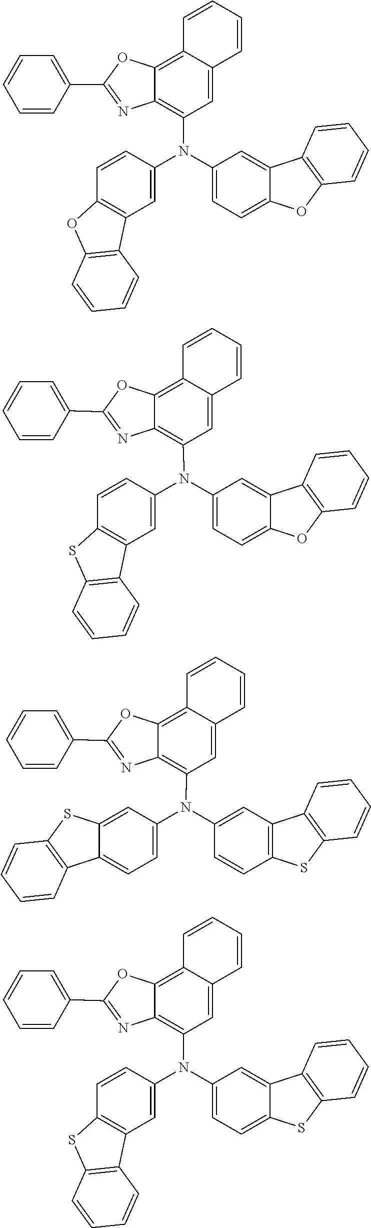 [SMILES: c1ccc(-c2nc3c(N(c4ccc5oc6ccccc6c5c4)c4ccc5oc6ccccc6c5c4)cc4ccccc4c3o2)cc1 ; c1ccc(-c2nc3c(N(c4ccc5oc6ccccc6c5c4)c4ccc5sc6ccccc6c5c4)cc4ccccc4c3o2)cc1 ; c1ccc(-c2nc3c(N(c4ccc5c(c4)sc4ccccc45)c4ccc5sc6ccccc6c5c4)cc4ccccc4c3o2)cc1 ; c1ccc(-c2nc3c(N(c4ccc5sc6ccccc6c5c4)c4ccc5sc6ccccc6c5c4)cc4ccccc4c3o2)cc1]

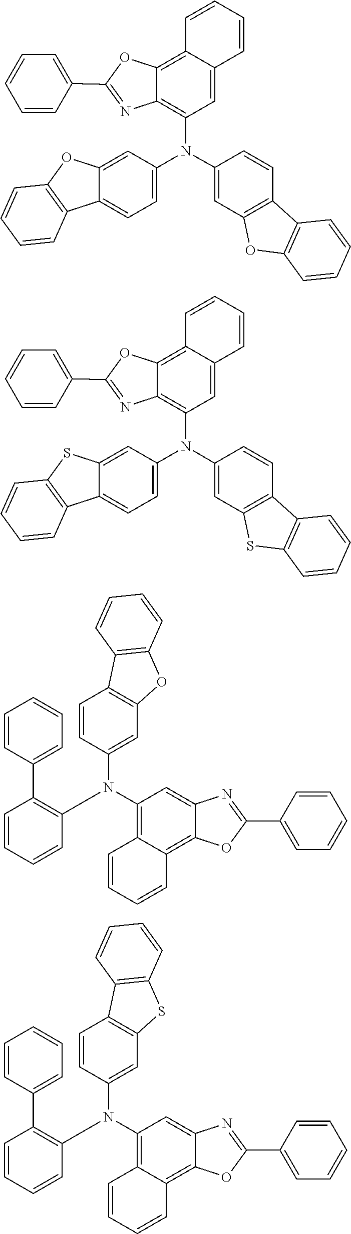

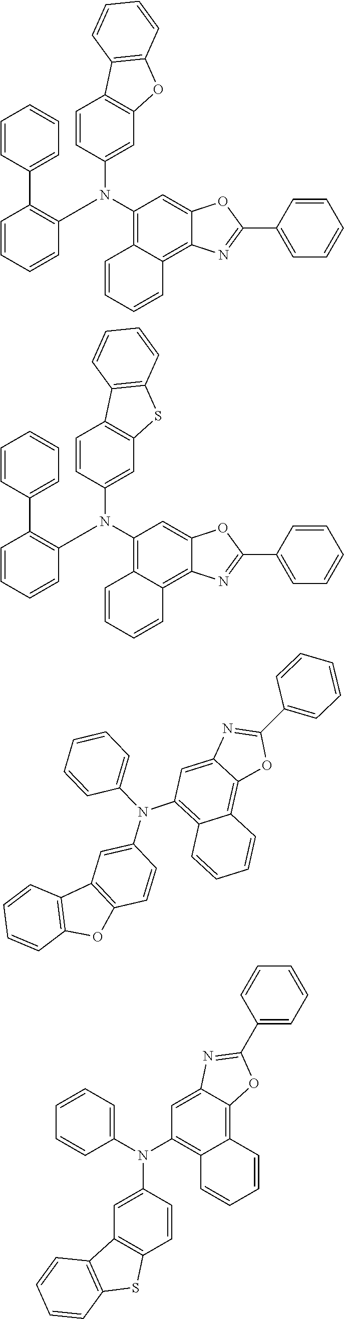

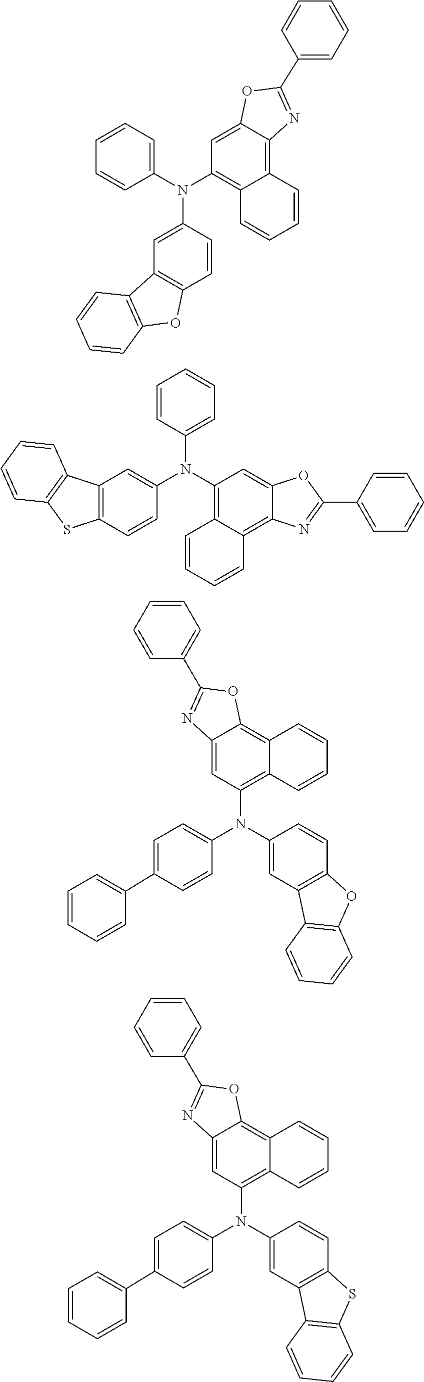

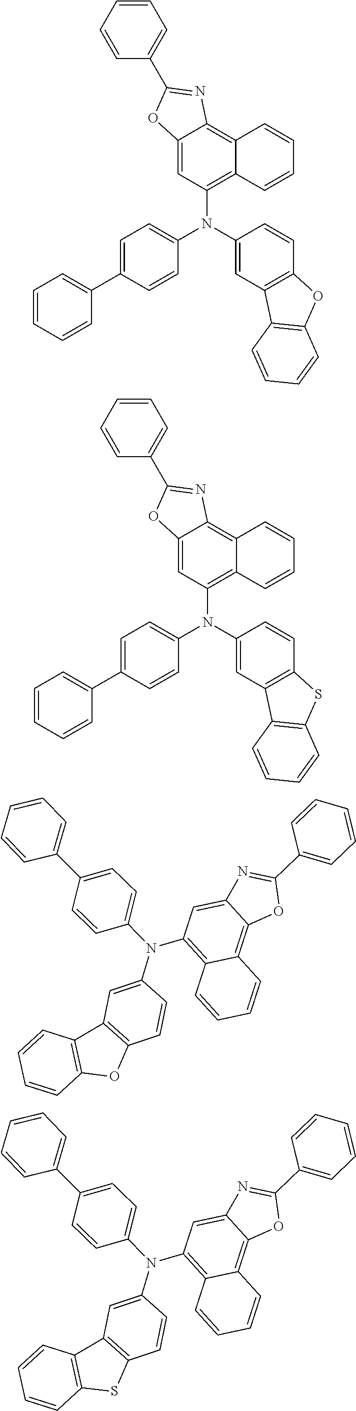

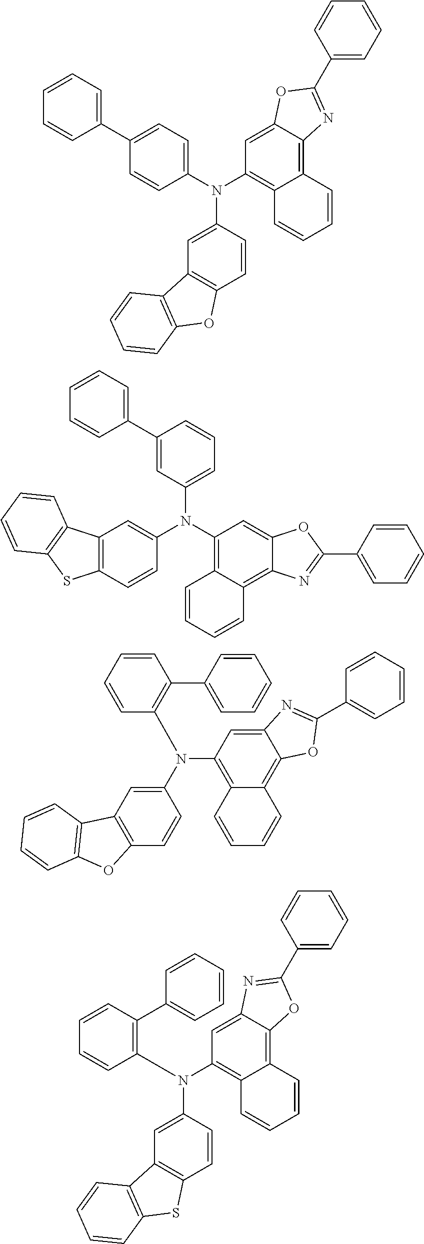

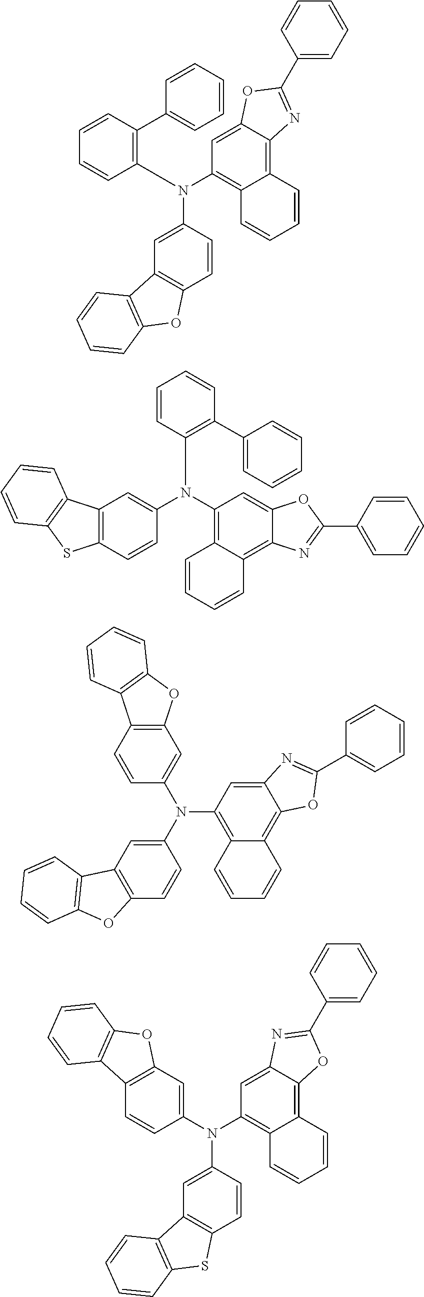 [SMILES: c1ccc(-c2nc3c(cc(N(c4ccc5oc6ccccc6c5c4)c4ccccc4-c4ccccc4)c4ccccc43)o2)cc1 ; c1ccc(-c2nc3c(cc(N(c4ccc5sc6ccccc6c5c4)c4ccccc4-c4ccccc4)c4ccccc43)o2)cc1 ; c1ccc(-c2nc3cc(N(c4ccc5c(c4)oc4ccccc45)c4ccc5oc6ccccc6c5c4)c4ccccc4c3o2)cc1 ; c1ccc(-c2nc3cc(N(c4ccc5c(c4)oc4ccccc45)c4ccc5sc6ccccc6c5c4)c4ccccc4c3o2)cc1]

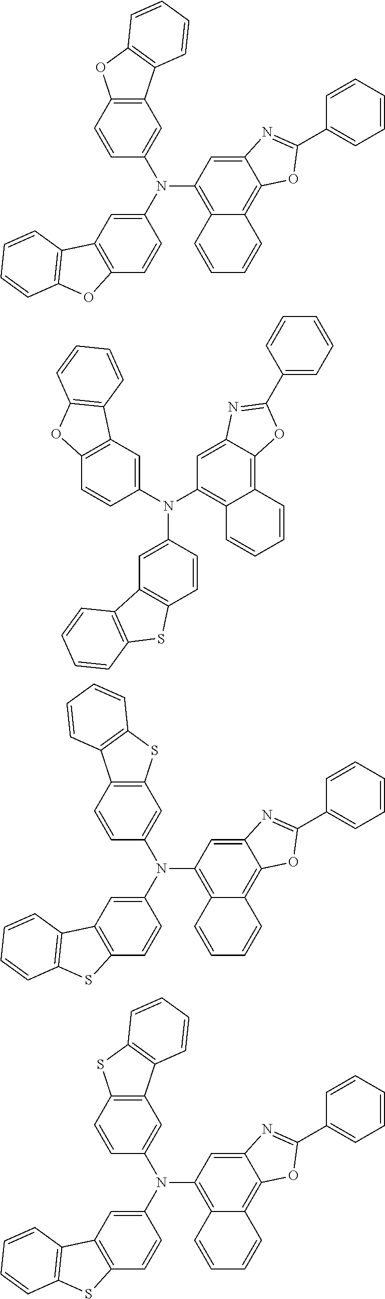

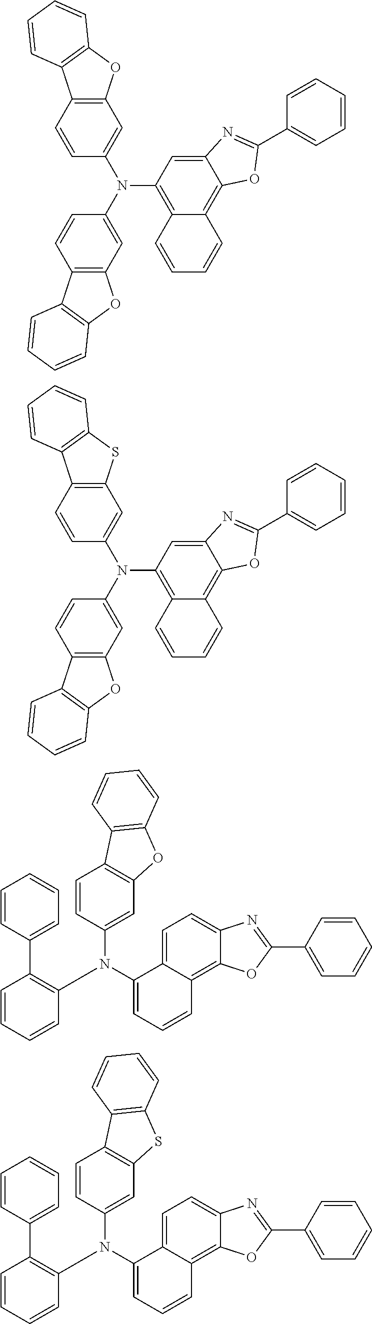

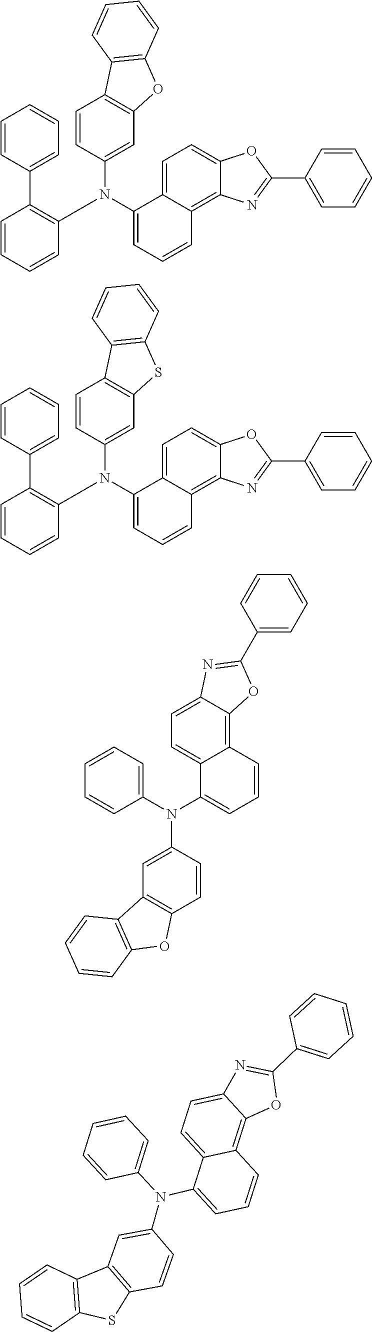

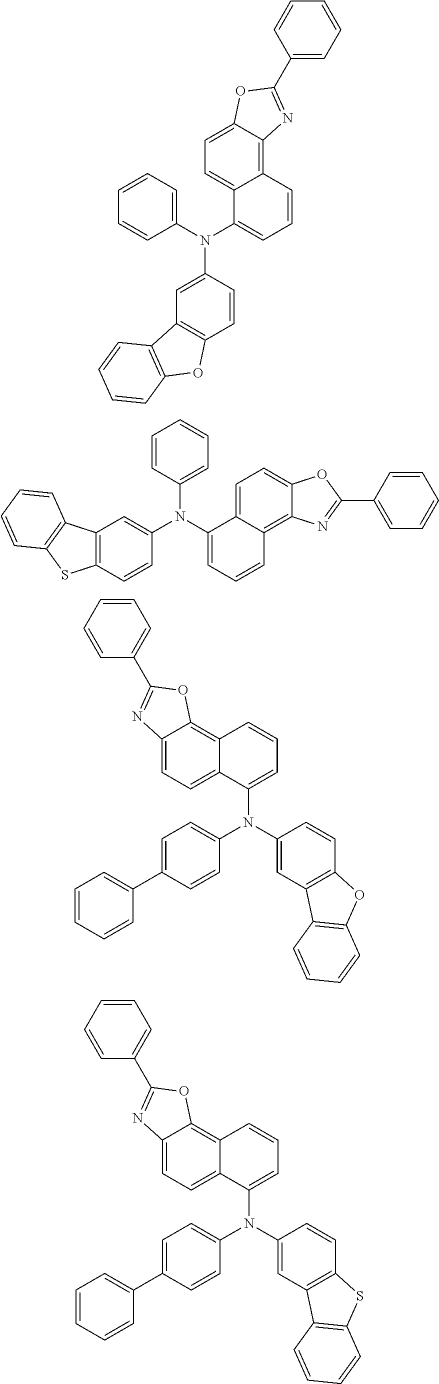

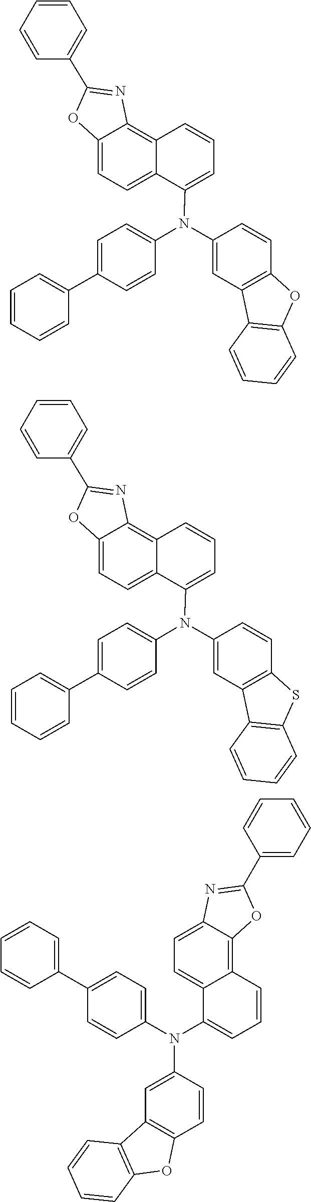

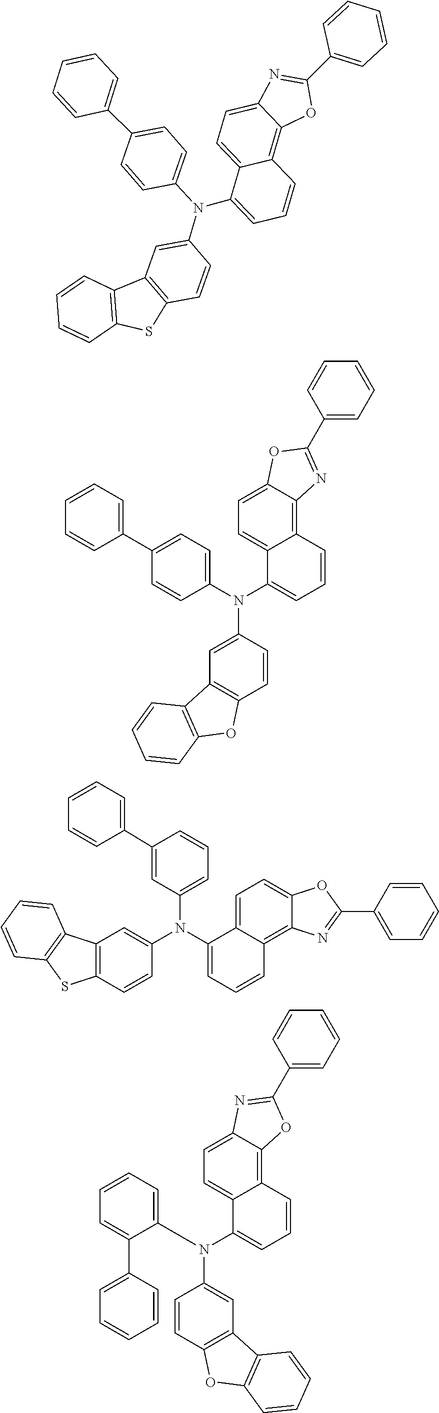

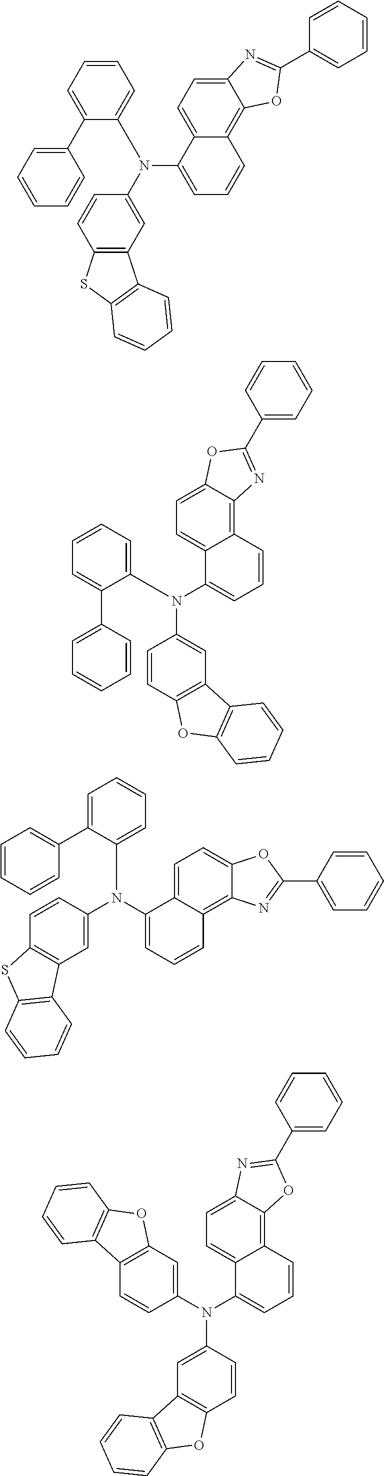 [SMILES: c1ccc(-c2nc3ccc4c(N(c5ccc6sc7ccccc7c6c5)c5ccccc5-c5ccccc5)cccc4c3o2)cc1 ; c1ccc(-c2nc3c(ccc4c(N(c5ccc6oc7ccccc7c6c5)c5ccccc5-c5ccccc5)cccc43)o2)cc1 ; c1ccc(-c2nc3c(ccc4c(N(c5ccc6sc7ccccc7c6c5)c5ccccc5-c5ccccc5)cccc43)o2)cc1 ; c1ccc(-c2nc3ccc4c(N(c5ccc6c(c5)oc5ccccc56)c5ccc6oc7ccccc7c6c5)cccc4c3o2)cc1]

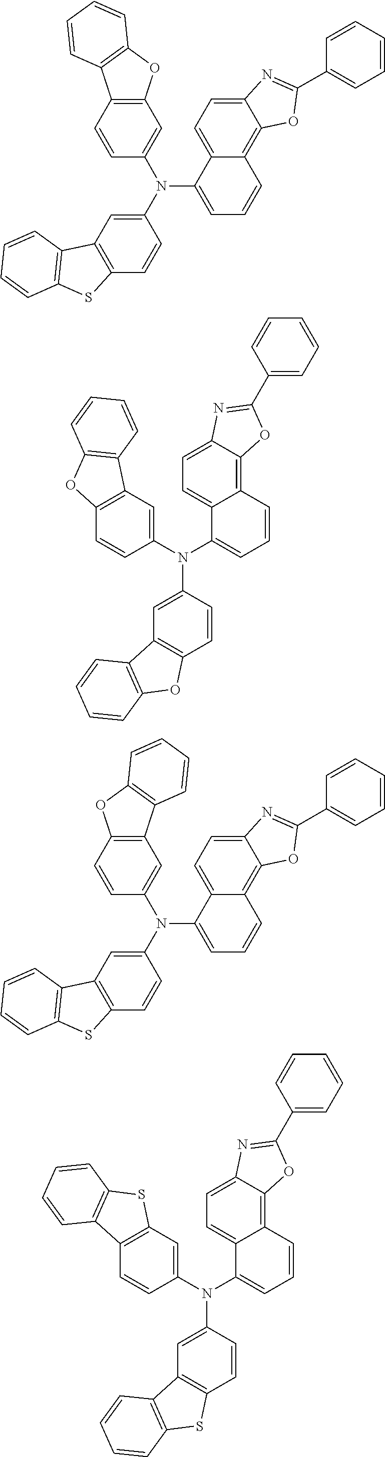

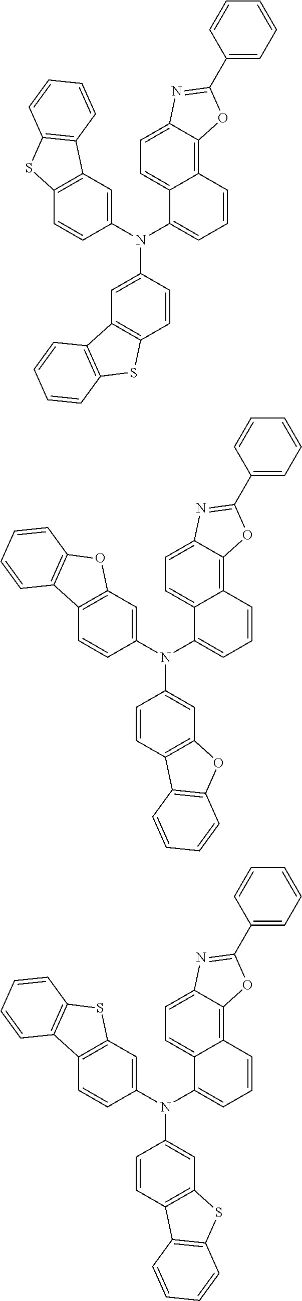 [SMILES: c1ccc(-c2nc3ccc4c(N(c5ccc6sc7ccccc7c6c5)c5ccc6sc7ccccc7c6c5)cccc4c3o2)cc1 ; c1ccc(-c2nc3ccc4c(N(c5ccc6c(c5)oc5ccccc56)c5ccc6c(c5)oc5ccccc56)cccc4c3o2)cc1 ; c1ccc(-c2nc3ccc4c(N(c5ccc6c(c5)sc5ccccc56)c5ccc6c(c5)sc5ccccc56)cccc4c3o2)cc1]

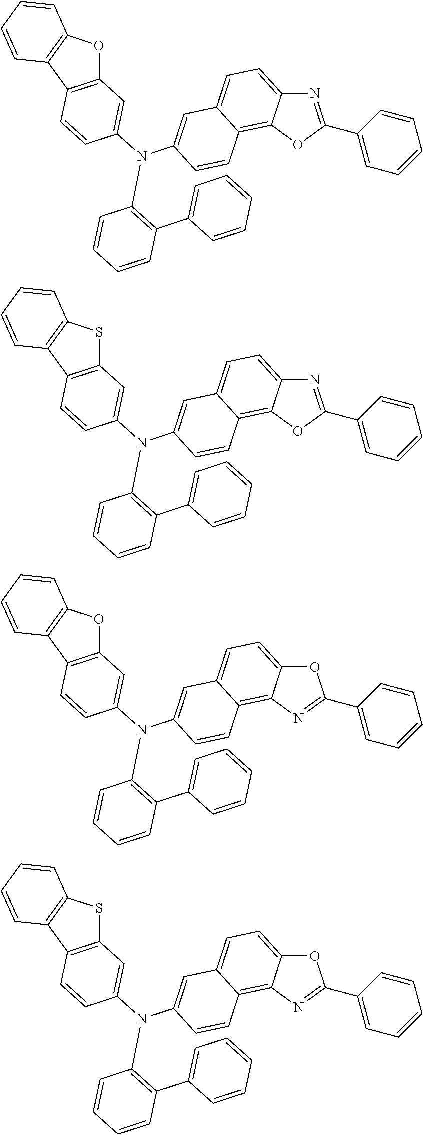 [SMILES: c1ccc(-c2nc3ccc4cc(N(c5ccc6c(c5)oc5ccccc56)c5ccccc5-c5ccccc5)ccc4c3o2)cc1 ; c1ccc(-c2nc3ccc4cc(N(c5ccc6c(c5)sc5ccccc56)c5ccccc5-c5ccccc5)ccc4c3o2)cc1 ; c1ccc(-c2nc3c(ccc4cc(N(c5ccc6c(c5)oc5ccccc56)c5ccccc5-c5ccccc5)ccc43)o2)cc1 ; c1ccc(-c2nc3c(ccc4cc(N(c5ccc6c(c5)sc5ccccc56)c5ccccc5-c5ccccc5)ccc43)o2)cc1]

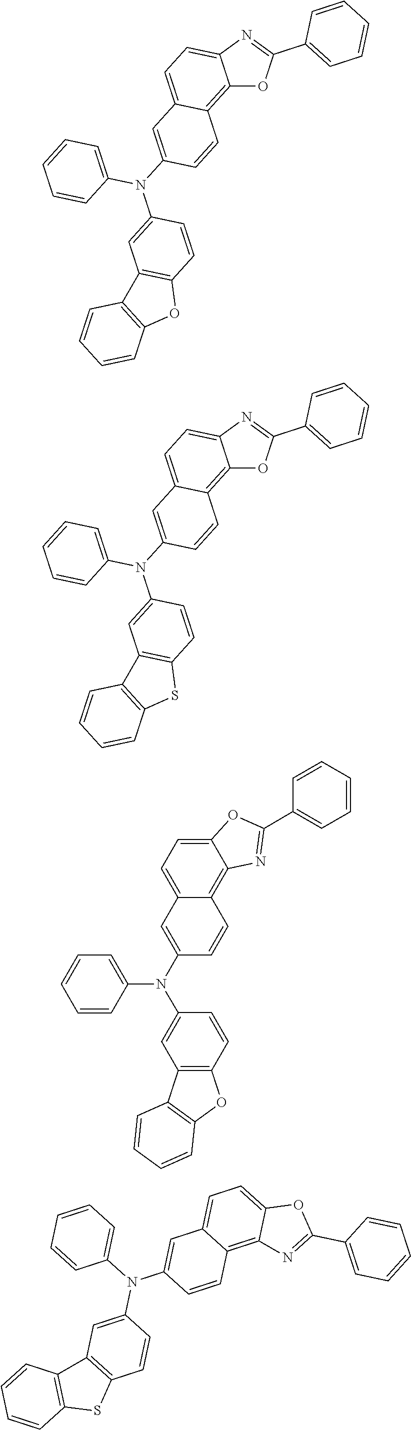

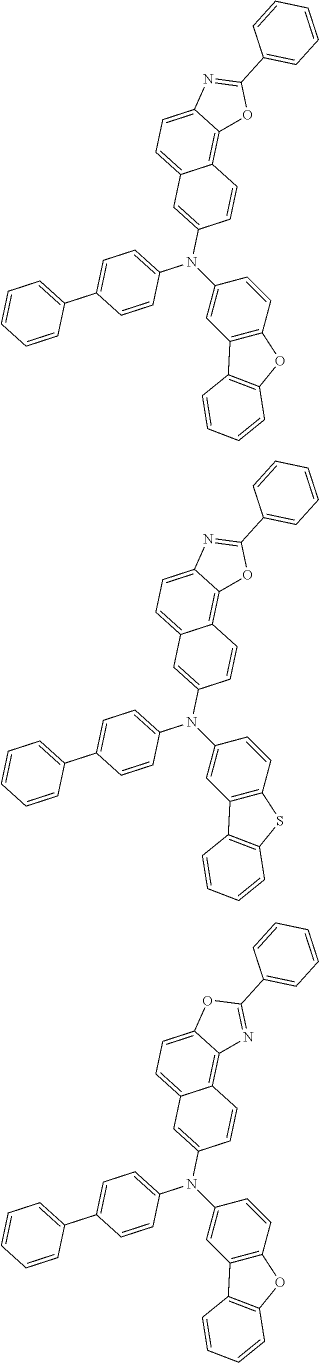

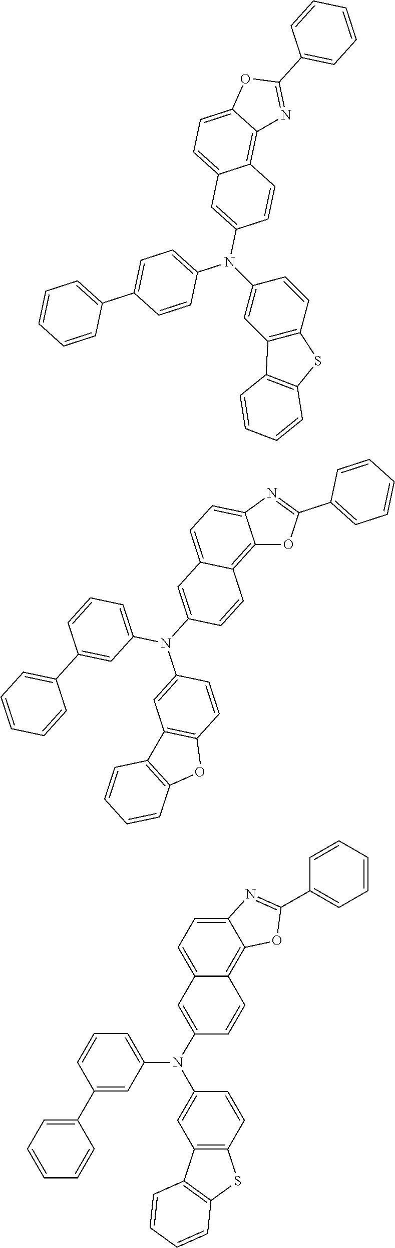 [SMILES: c1ccc(-c2ccc(N(c3ccc4c(ccc5oc(-c6ccccc6)nc54)c3)c3ccc4sc5ccccc5c4c3)cc2)cc1 ; c1ccc(-c2cccc(N(c3ccc4c(ccc5nc(-c6ccccc6)oc54)c3)c3ccc4oc5ccccc5c4c3)c2)cc1 ; c1ccc(-c2cccc(N(c3ccc4c(ccc5nc(-c6ccccc6)oc54)c3)c3ccc4sc5ccccc5c4c3)c2)cc1]

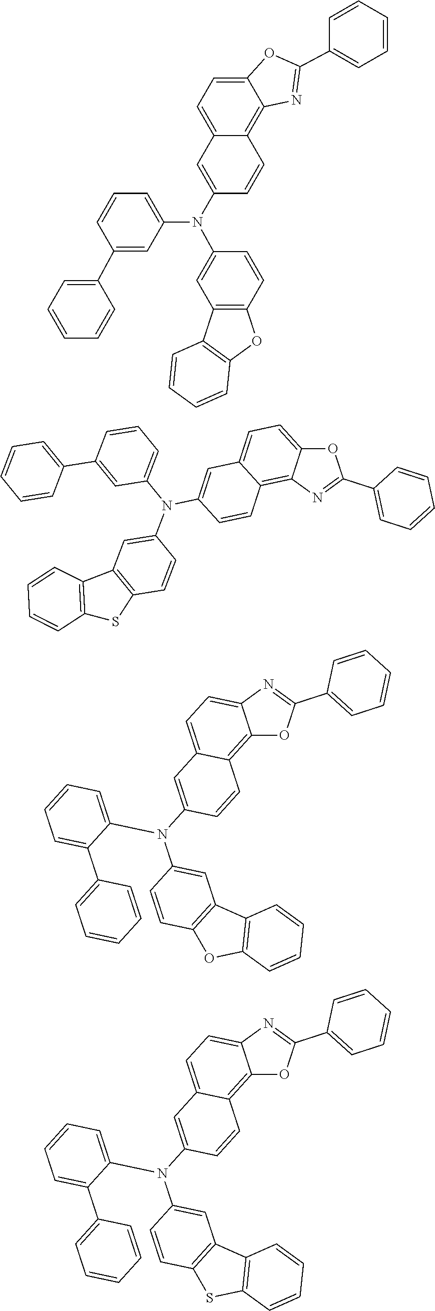

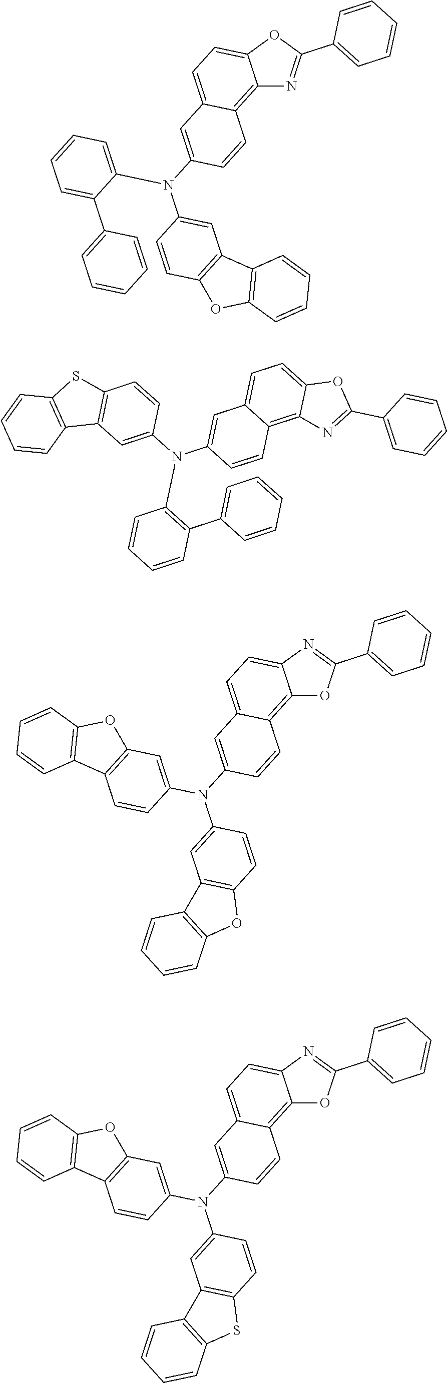

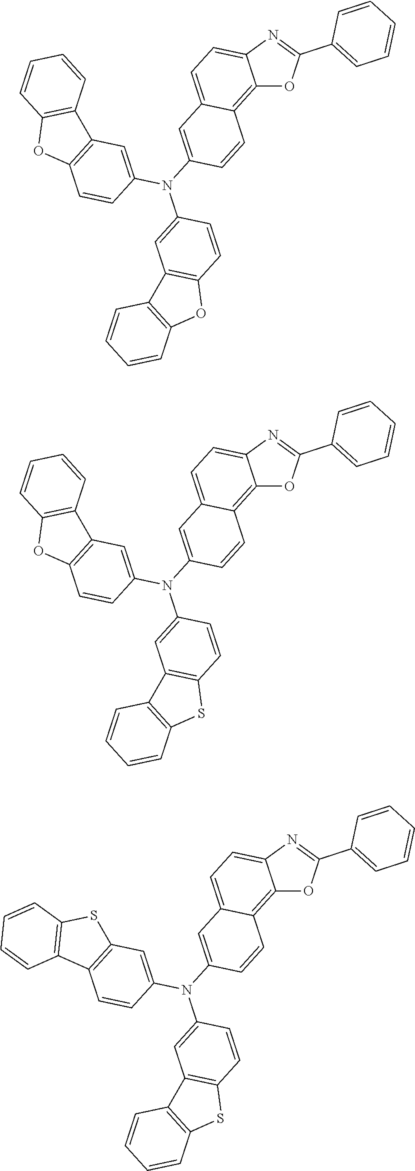 [SMILES: c1ccc(-c2nc3ccc4cc(N(c5ccc6oc7ccccc7c6c5)c5ccc6oc7ccccc7c6c5)ccc4c3o2)cc1 ; c1ccc(-c2nc3ccc4cc(N(c5ccc6oc7ccccc7c6c5)c5ccc6sc7ccccc7c6c5)ccc4c3o2)cc1 ; c1ccc(-c2nc3ccc4cc(N(c5ccc6c(c5)sc5ccccc56)c5ccc6sc7ccccc7c6c5)ccc4c3o2)cc1]

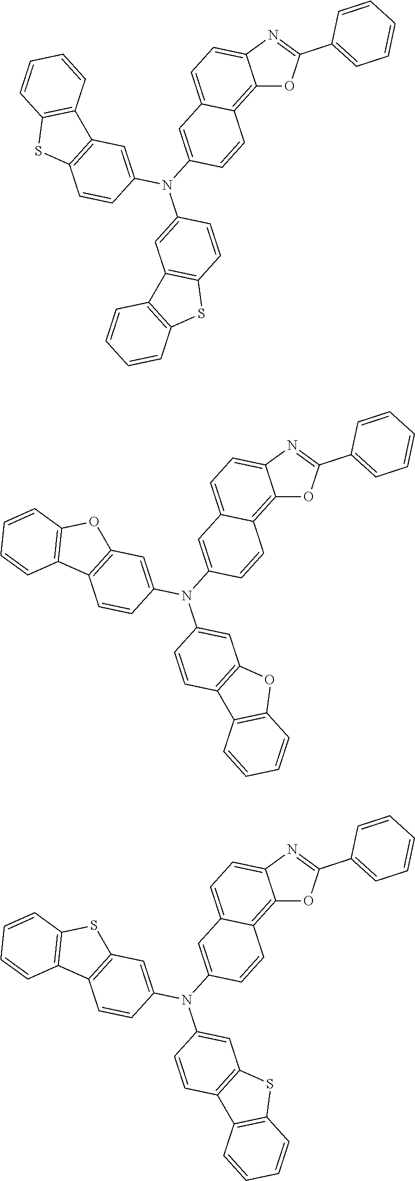 [SMILES: c1ccc(-c2nc3ccc4cc(N(c5ccc6sc7ccccc7c6c5)c5ccc6sc7ccccc7c6c5)ccc4c3o2)cc1 ; c1ccc(-c2nc3ccc4cc(N(c5ccc6c(c5)oc5ccccc56)c5ccc6c(c5)oc5ccccc56)ccc4c3o2)cc1 ; c1ccc(-c2nc3ccc4cc(N(c5ccc6c(c5)sc5ccccc56)c5ccc6c(c5)sc5ccccc56)ccc4c3o2)cc1]

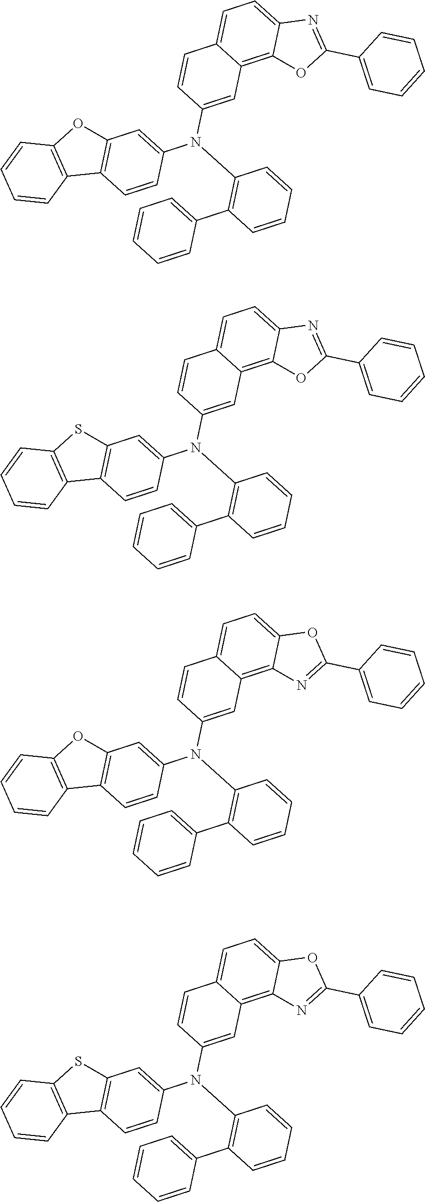 [SMILES: c1ccc(-c2nc3ccc4ccc(N(c5ccc6c(c5)oc5ccccc56)c5ccccc5-c5ccccc5)cc4c3o2)cc1 ; c1ccc(-c2nc3ccc4ccc(N(c5ccc6c(c5)sc5ccccc56)c5ccccc5-c5ccccc5)cc4c3o2)cc1 ; c1ccc(-c2nc3c(ccc4ccc(N(c5ccc6c(c5)oc5ccccc56)c5ccccc5-c5ccccc5)cc43)o2)cc1 ; c1ccc(-c2nc3c(ccc4ccc(N(c5ccc6c(c5)sc5ccccc56)c5ccccc5-c5ccccc5)cc43)o2)cc1]

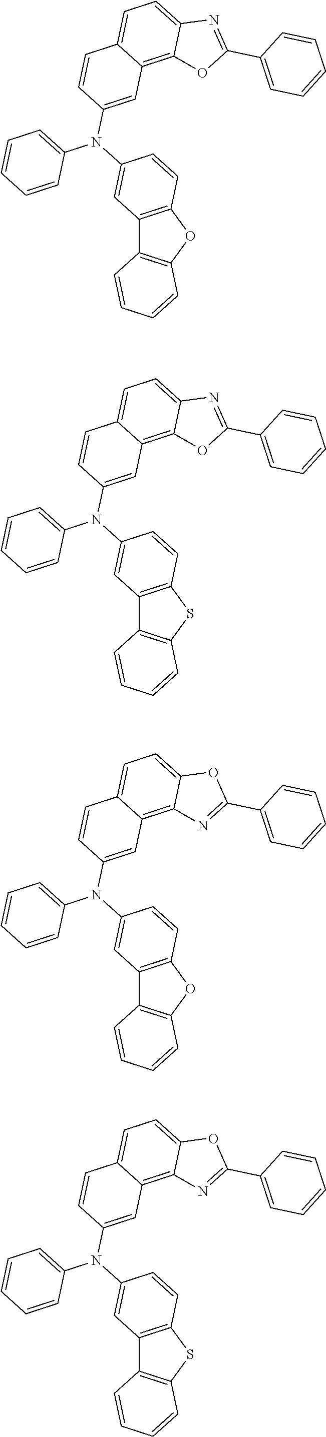 [SMILES: c1ccc(-c2nc3ccc4ccc(N(c5ccccc5)c5ccc6oc7ccccc7c6c5)cc4c3o2)cc1 ; c1ccc(-c2nc3ccc4ccc(N(c5ccccc5)c5ccc6sc7ccccc7c6c5)cc4c3o2)cc1 ; c1ccc(-c2nc3c(ccc4ccc(N(c5ccccc5)c5ccc6oc7ccccc7c6c5)cc43)o2)cc1 ; c1ccc(-c2nc3c(ccc4ccc(N(c5ccccc5)c5ccc6sc7ccccc7c6c5)cc43)o2)cc1]

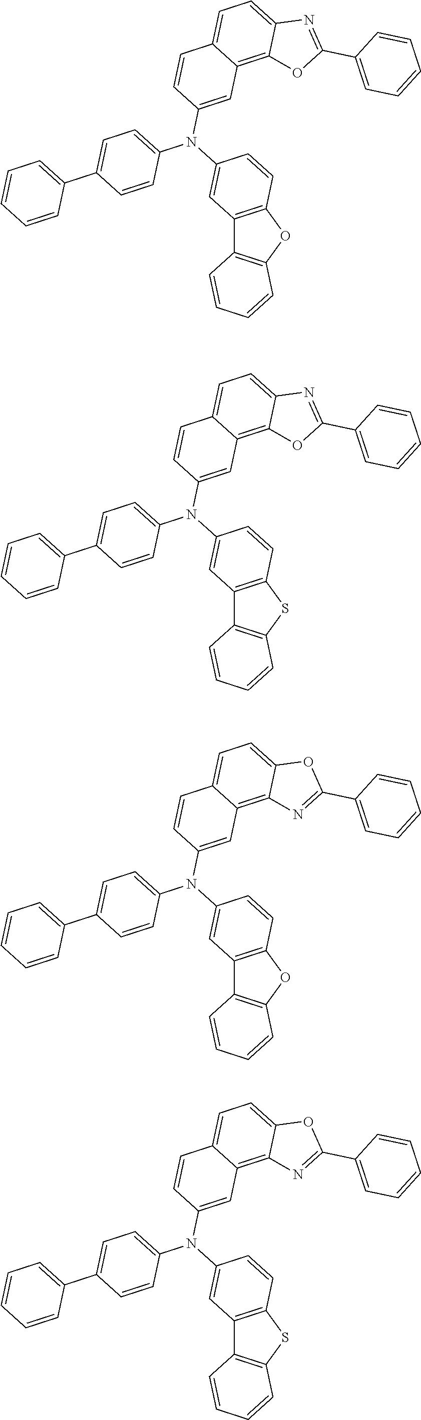

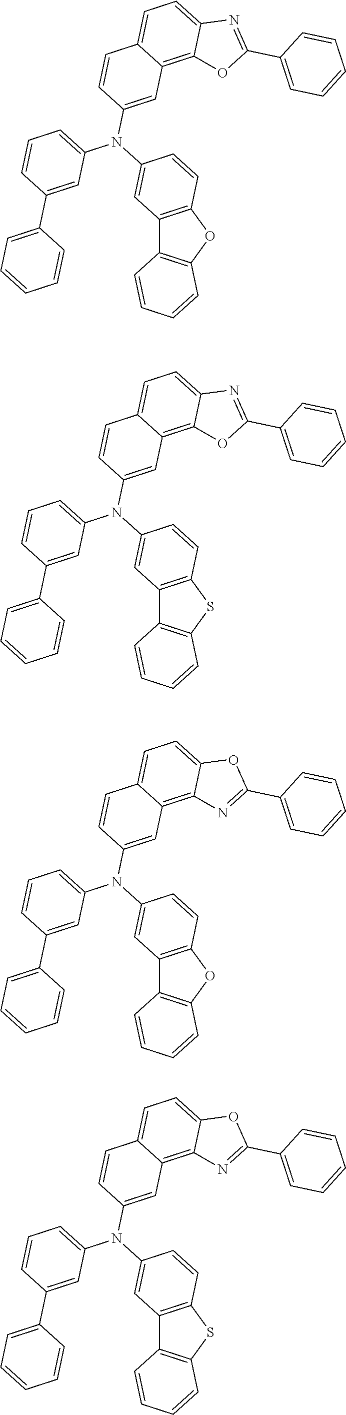

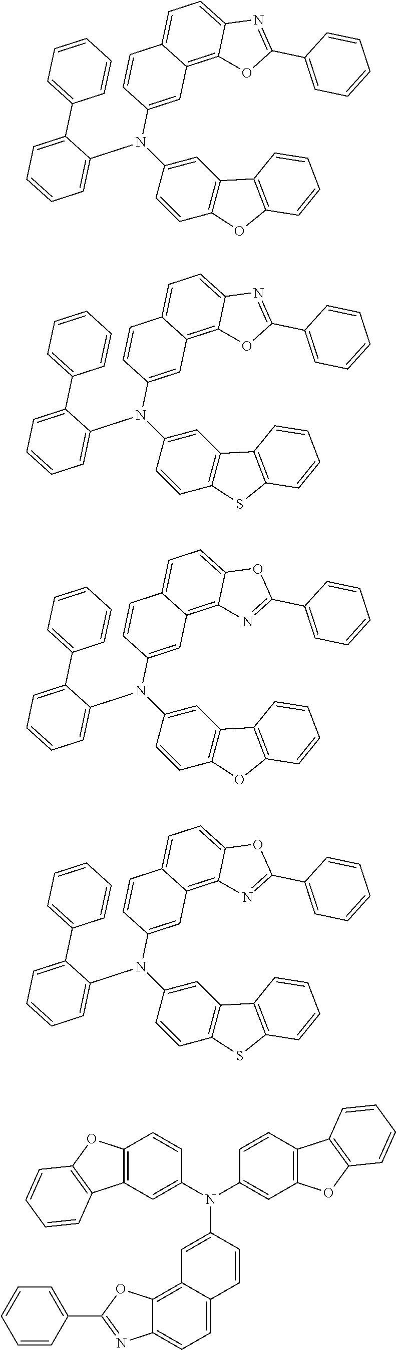 [SMILES: c1ccc(-c2nc3ccc4ccc(N(c5ccc6oc7ccccc7c6c5)c5ccccc5-c5ccccc5)cc4c3o2)cc1 ; c1ccc(-c2nc3ccc4ccc(N(c5ccc6sc7ccccc7c6c5)c5ccccc5-c5ccccc5)cc4c3o2)cc1 ; c1ccc(-c2nc3c(ccc4ccc(N(c5ccc6oc7ccccc7c6c5)c5ccccc5-c5ccccc5)cc43)o2)cc1 ; c1ccc(-c2nc3c(ccc4ccc(N(c5ccc6sc7ccccc7c6c5)c5ccccc5-c5ccccc5)cc43)o2)cc1 ; c1ccc(-c2nc3ccc4ccc(N(c5ccc6c(c5)oc5ccccc56)c5ccc6oc7ccccc7c6c5)cc4c3o2)cc1]

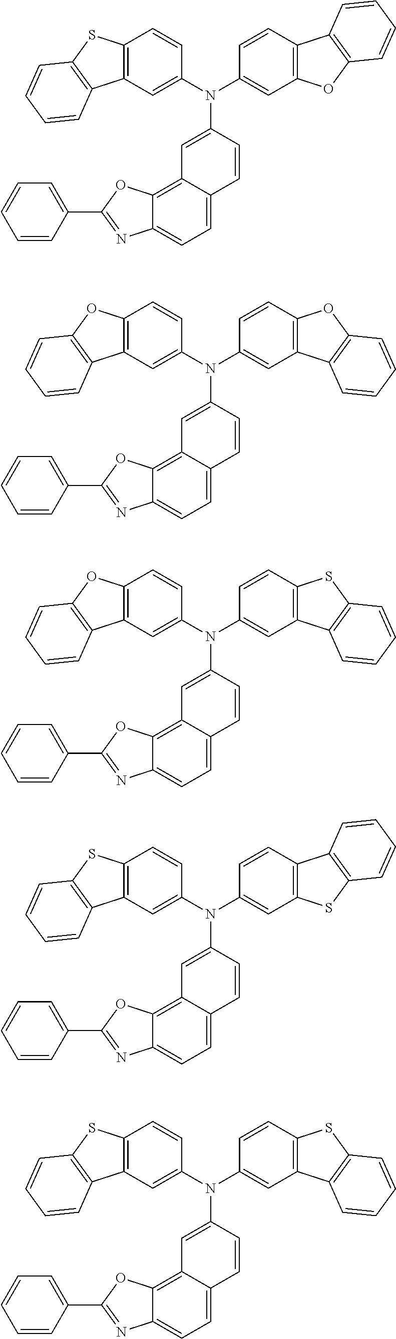 [SMILES: c1ccc(-c2nc3ccc4ccc(N(c5ccc6c(c5)oc5ccccc56)c5ccc6sc7ccccc7c6c5)cc4c3o2)cc1 ; c1ccc(-c2nc3ccc4ccc(N(c5ccc6oc7ccccc7c6c5)c5ccc6oc7ccccc7c6c5)cc4c3o2)cc1 ; c1ccc(-c2nc3ccc4ccc(N(c5ccc6oc7ccccc7c6c5)c5ccc6sc7ccccc7c6c5)cc4c3o2)cc1 ; c1ccc(-c2nc3ccc4ccc(N(c5ccc6c(c5)sc5ccccc56)c5ccc6sc7ccccc7c6c5)cc4c3o2)cc1 ; c1ccc(-c2nc3ccc4ccc(N(c5ccc6sc7ccccc7c6c5)c5ccc6sc7ccccc7c6c5)cc4c3o2)cc1]

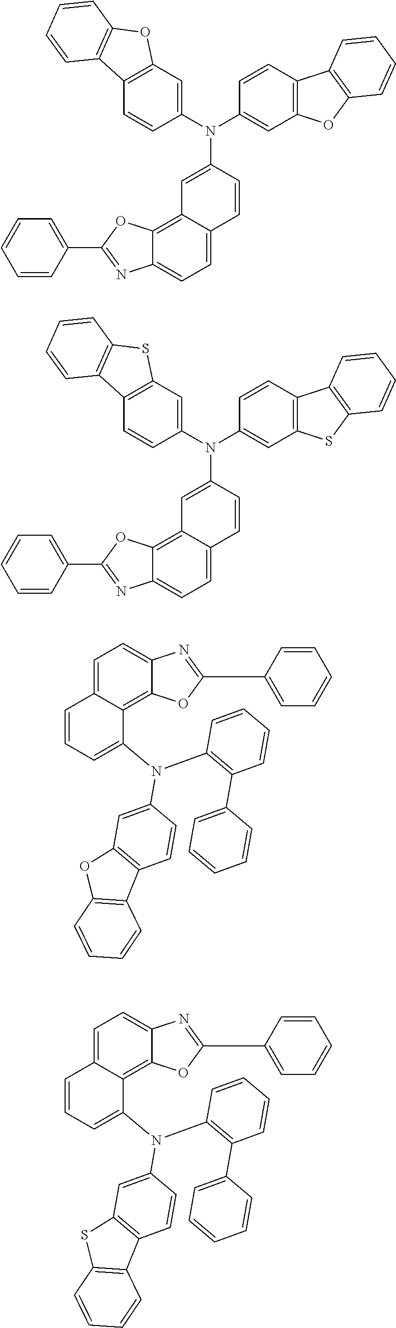

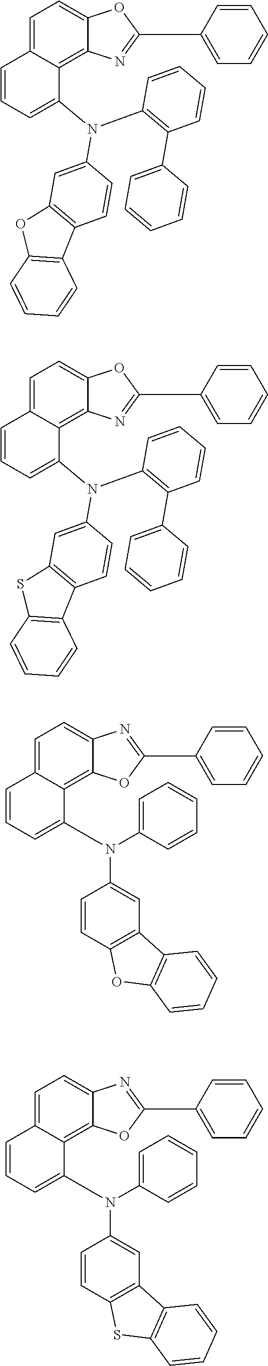

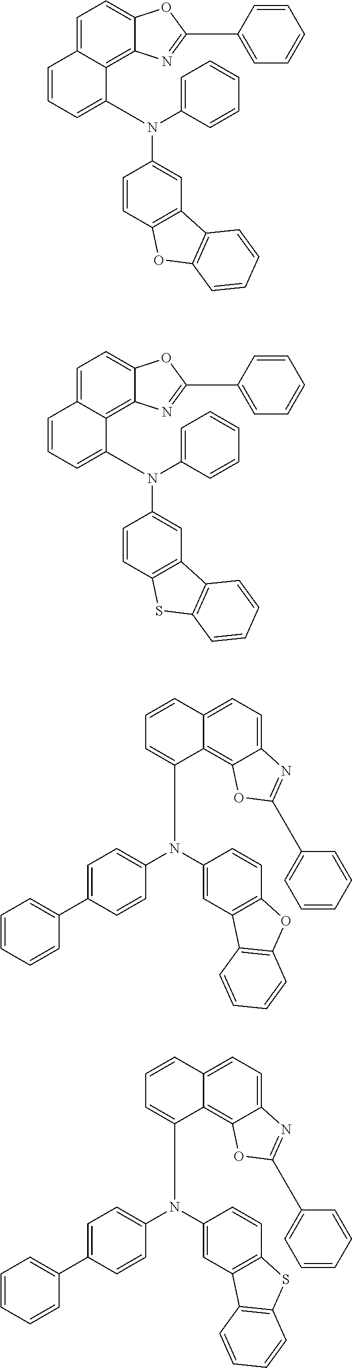

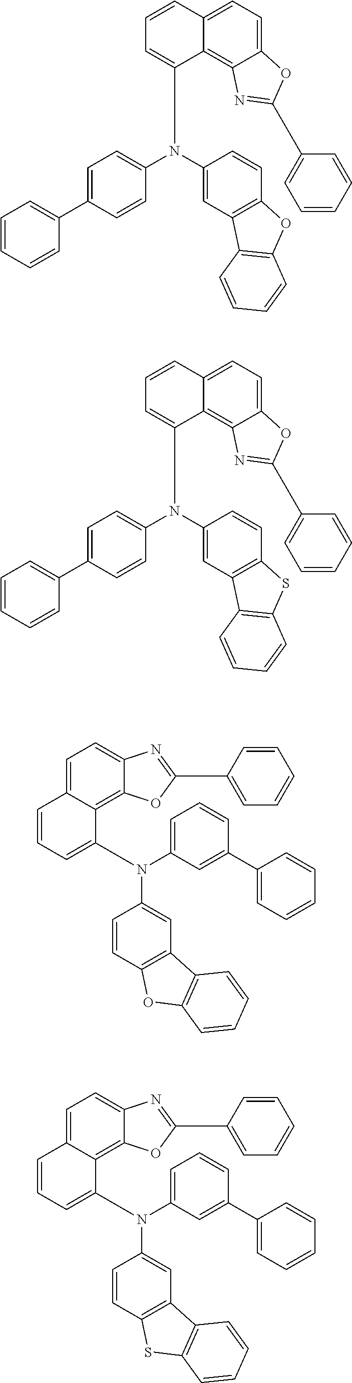 [SMILES: c1ccc(-c2ccc(N(c3ccc4oc5ccccc5c4c3)c3cccc4ccc5oc(-c6ccccc6)nc5c34)cc2)cc1 ; c1ccc(-c2ccc(N(c3ccc4sc5ccccc5c4c3)c3cccc4ccc5oc(-c6ccccc6)nc5c34)cc2)cc1 ; c1ccc(-c2cccc(N(c3ccc4oc5ccccc5c4c3)c3cccc4ccc5nc(-c6ccccc6)oc5c34)c2)cc1 ; c1ccc(-c2cccc(N(c3ccc4sc5ccccc5c4c3)c3cccc4ccc5nc(-c6ccccc6)oc5c34)c2)cc1]

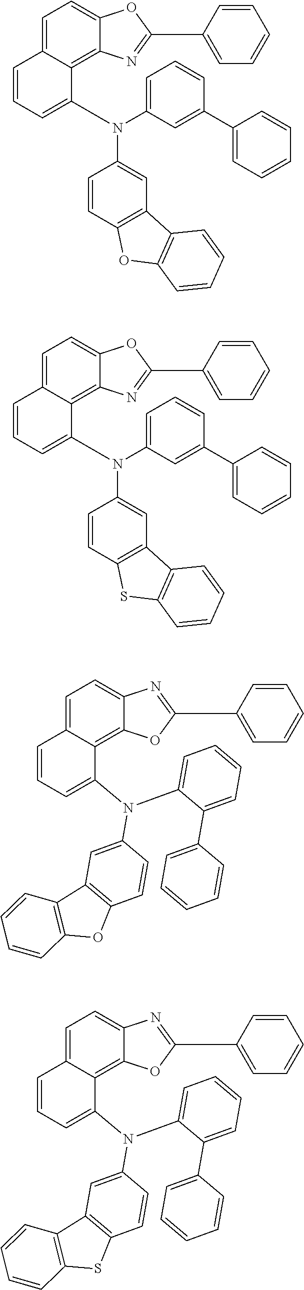 [SMILES: c1ccc(-c2cccc(N(c3ccc4oc5ccccc5c4c3)c3cccc4ccc5oc(-c6ccccc6)nc5c34)c2)cc1 ; c1ccc(-c2cccc(N(c3ccc4sc5ccccc5c4c3)c3cccc4ccc5oc(-c6ccccc6)nc5c34)c2)cc1 ; c1ccc(-c2nc3ccc4cccc(N(c5ccc6oc7ccccc7c6c5)c5ccccc5-c5ccccc5)c4c3o2)cc1 ; c1ccc(-c2nc3ccc4cccc(N(c5ccc6sc7ccccc7c6c5)c5ccccc5-c5ccccc5)c4c3o2)cc1]

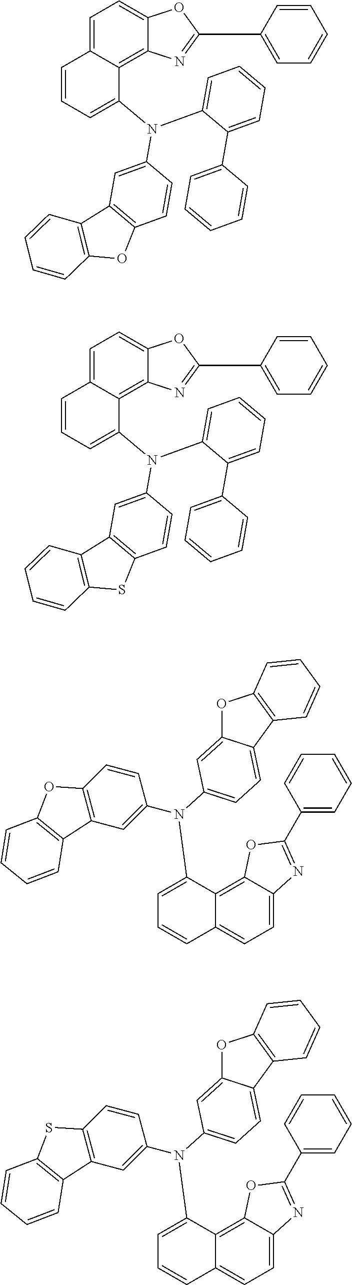 [SMILES: c1ccc(-c2nc3c(ccc4cccc(N(c5ccc6oc7ccccc7c6c5)c5ccccc5-c5ccccc5)c43)o2)cc1 ; c1ccc(-c2nc3c(ccc4cccc(N(c5ccc6sc7ccccc7c6c5)c5ccccc5-c5ccccc5)c43)o2)cc1 ; c1ccc(-c2nc3ccc4cccc(N(c5ccc6c(c5)oc5ccccc56)c5ccc6oc7ccccc7c6c5)c4c3o2)cc1 ; c1ccc(-c2nc3ccc4cccc(N(c5ccc6c(c5)oc5ccccc56)c5ccc6sc7ccccc7c6c5)c4c3o2)cc1]

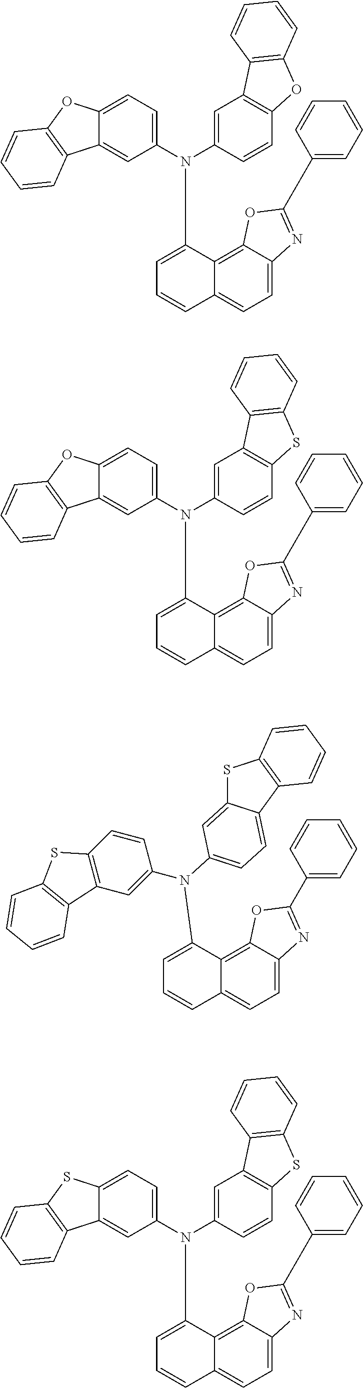

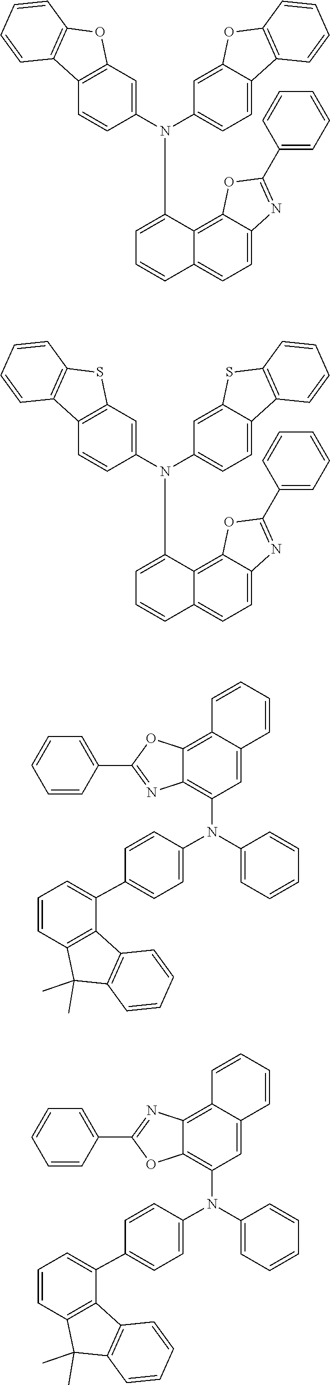 [SMILES: c1ccc(-c2nc3ccc4cccc(N(c5ccc6c(c5)oc5ccccc56)c5ccc6c(c5)oc5ccccc56)c4c3o2)cc1 ; c1ccc(-c2nc3ccc4cccc(N(c5ccc6c(c5)sc5ccccc56)c5ccc6c(c5)sc5ccccc56)c4c3o2)cc1 ; CC1(C)c2ccccc2-c2c(-c3ccc(N(c4ccccc4)c4cc5ccccc5c5oc(-c6ccccc6)nc45)cc3)cccc21 ; CC1(C)c2ccccc2-c2c(-c3ccc(N(c4ccccc4)c4cc5ccccc5c5nc(-c6ccccc6)oc45)cc3)cccc21]

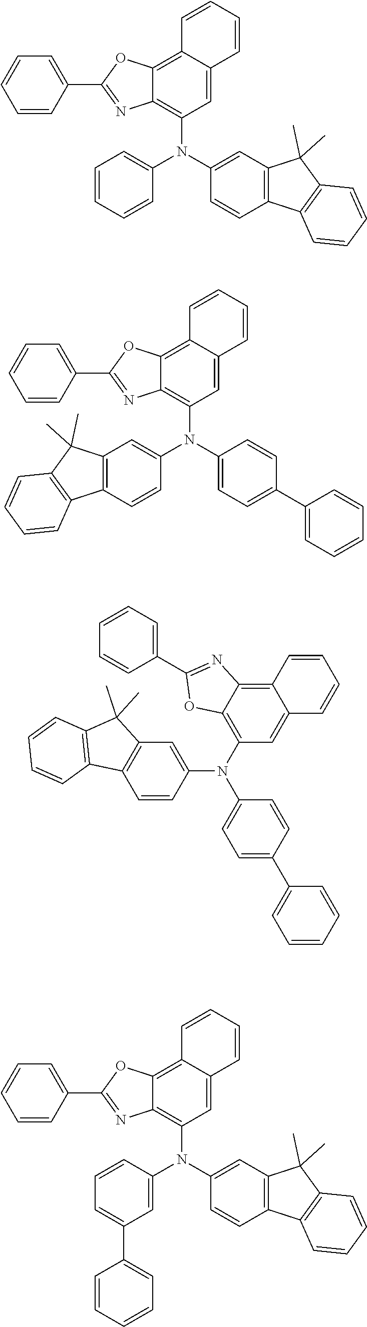 [SMILES: CC1(C)c2ccccc2-c2ccc(N(c3ccccc3)c3cc4ccccc4c4oc(-c5ccccc5)nc34)cc21 ; CC1(C)c2ccccc2-c2ccc(N(c3ccc(-c4ccccc4)cc3)c3cc4ccccc4c4oc(-c5ccccc5)nc34)cc21 ; CC1(C)c2ccccc2-c2ccc(N(c3ccc(-c4ccccc4)cc3)c3cc4ccccc4c4nc(-c5ccccc5)oc34)cc21 ; CC1(C)c2ccccc2-c2ccc(N(c3cccc(-c4ccccc4)c3)c3cc4ccccc4c4oc(-c5ccccc5)nc34)cc21]

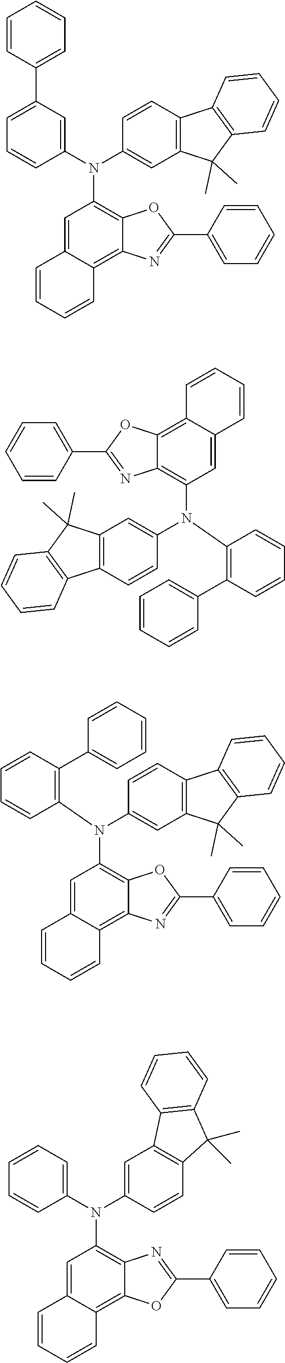 [SMILES: CC1(C)c2ccccc2-c2ccc(N(c3cccc(-c4ccccc4)c3)c3cc4ccccc4c4nc(-c5ccccc5)oc34)cc21 ; CC1(C)c2ccccc2-c2ccc(N(c3ccccc3-c3ccccc3)c3cc4ccccc4c4oc(-c5ccccc5)nc34)cc21 ; CC1(C)c2ccccc2-c2ccc(N(c3ccccc3-c3ccccc3)c3cc4ccccc4c4nc(-c5ccccc5)oc34)cc21 ; CC1(C)c2ccccc2-c2cc(N(c3ccccc3)c3cc4ccccc4c4oc(-c5ccccc5)nc34)ccc21]

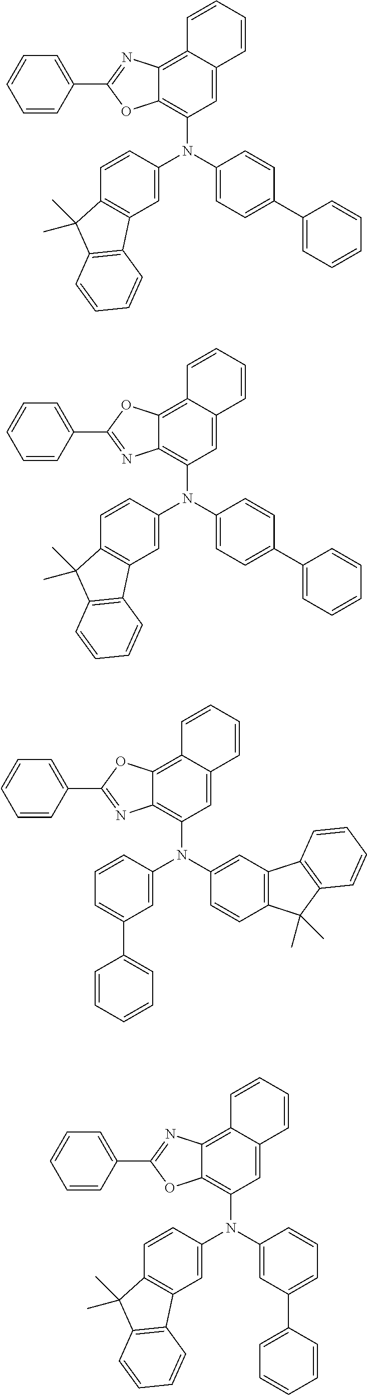 [SMILES: CC1(C)c2ccccc2-c2cc(N(c3ccc(-c4ccccc4)cc3)c3cc4ccccc4c4nc(-c5ccccc5)oc34)ccc21 ; CC1(C)c2ccccc2-c2cc(N(c3ccc(-c4ccccc4)cc3)c3cc4ccccc4c4oc(-c5ccccc5)nc34)ccc21 ; CC1(C)c2ccccc2-c2cc(N(c3cccc(-c4ccccc4)c3)c3cc4ccccc4c4oc(-c5ccccc5)nc34)ccc21 ; CC1(C)c2ccccc2-c2cc(N(c3cccc(-c4ccccc4)c3)c3cc4ccccc4c4nc(-c5ccccc5)oc34)ccc21]

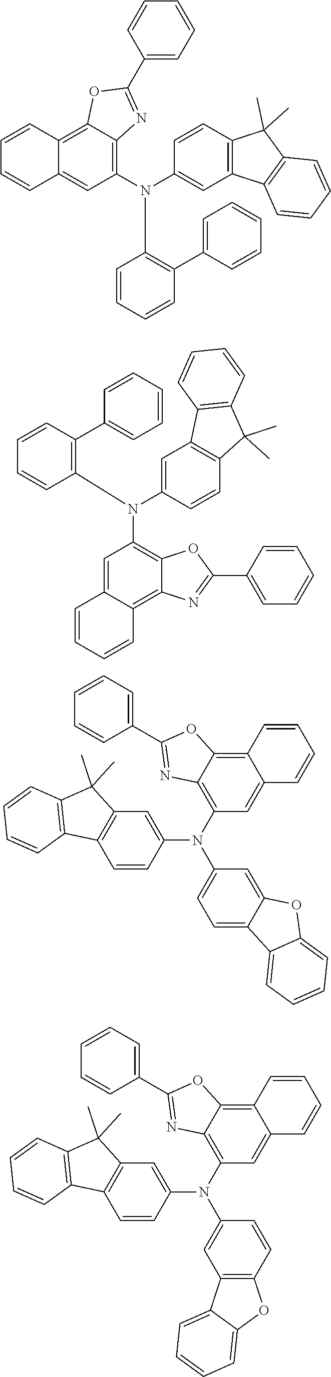

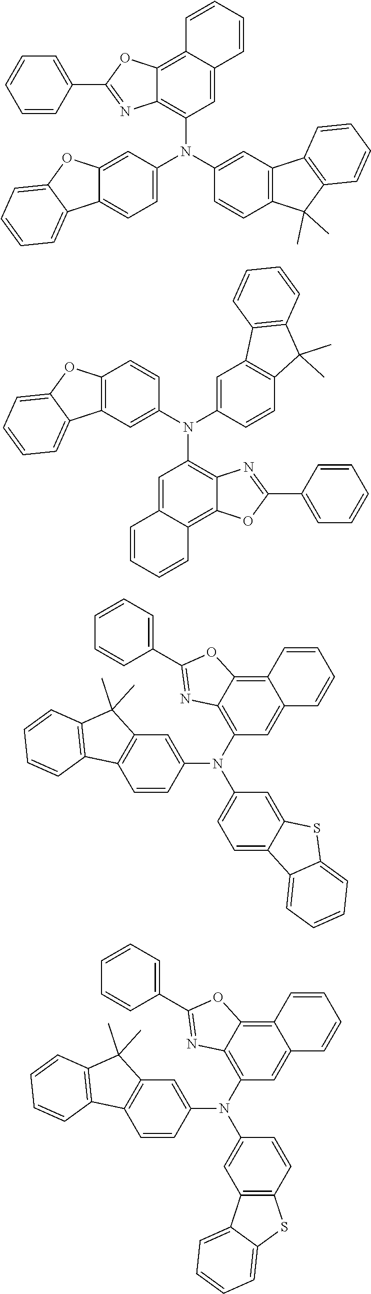

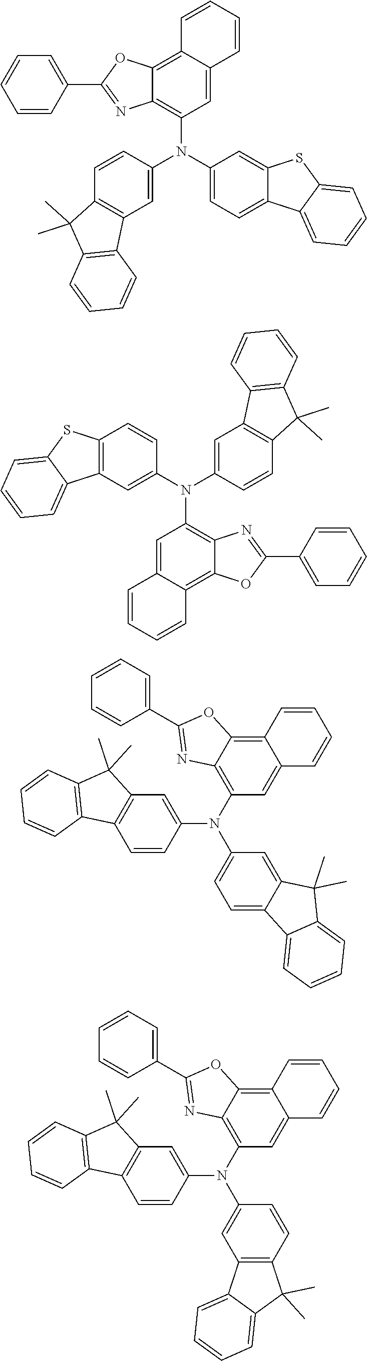

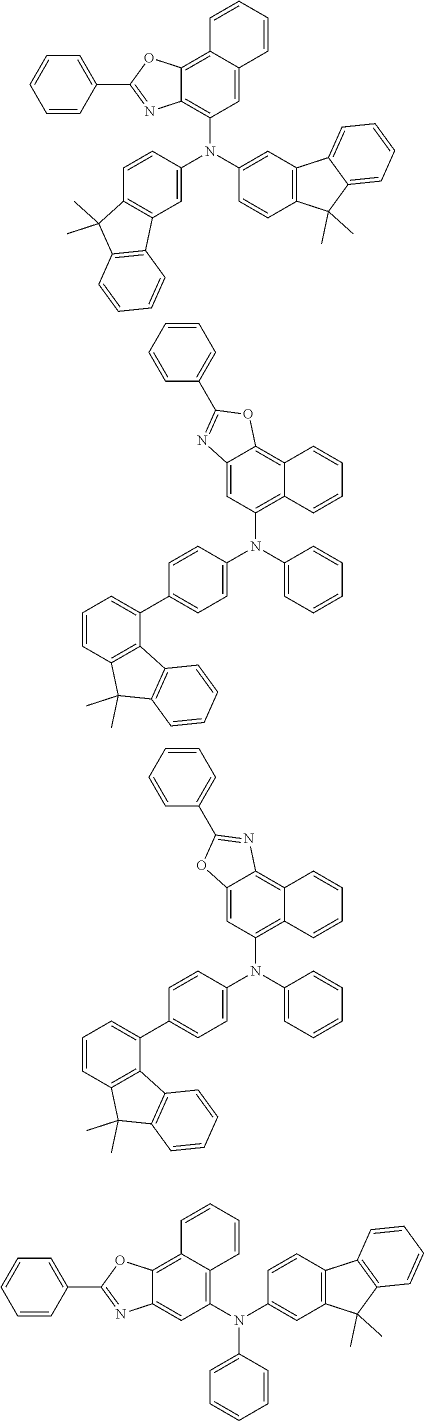

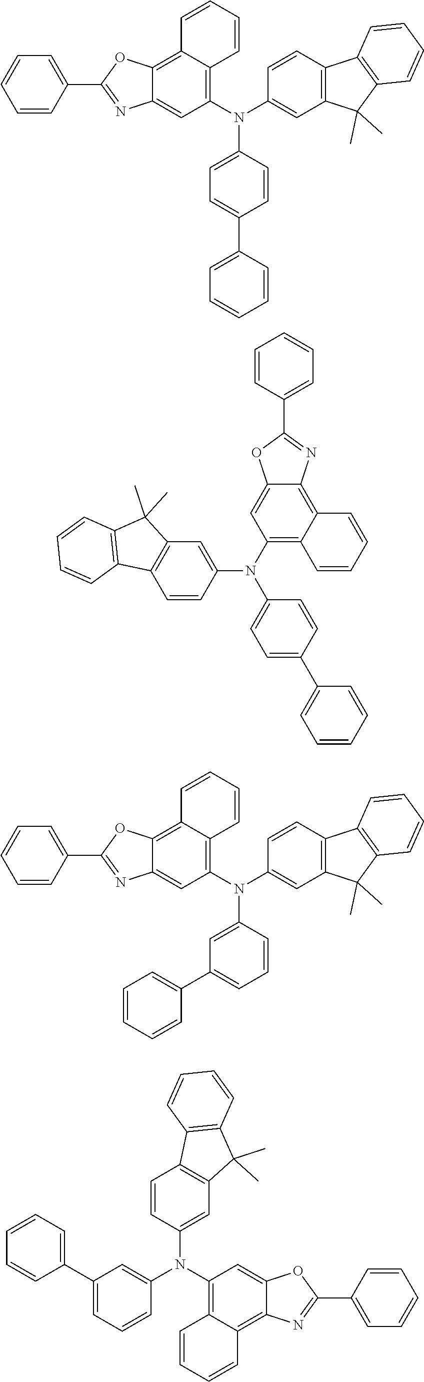

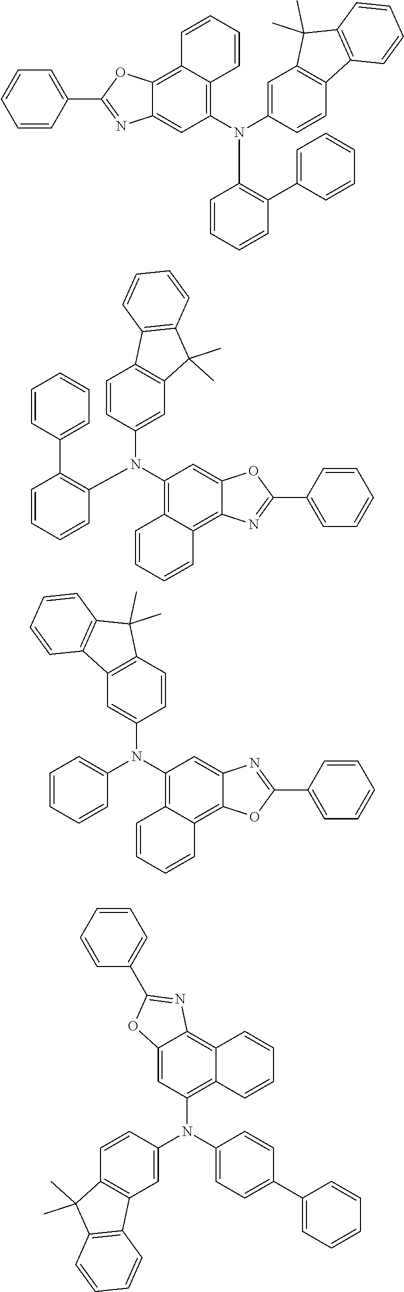

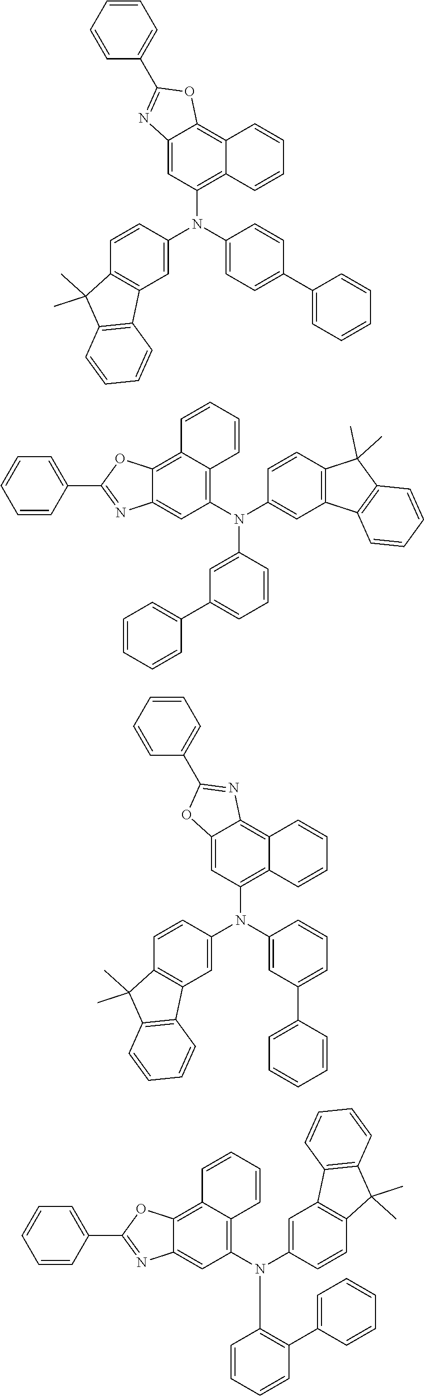

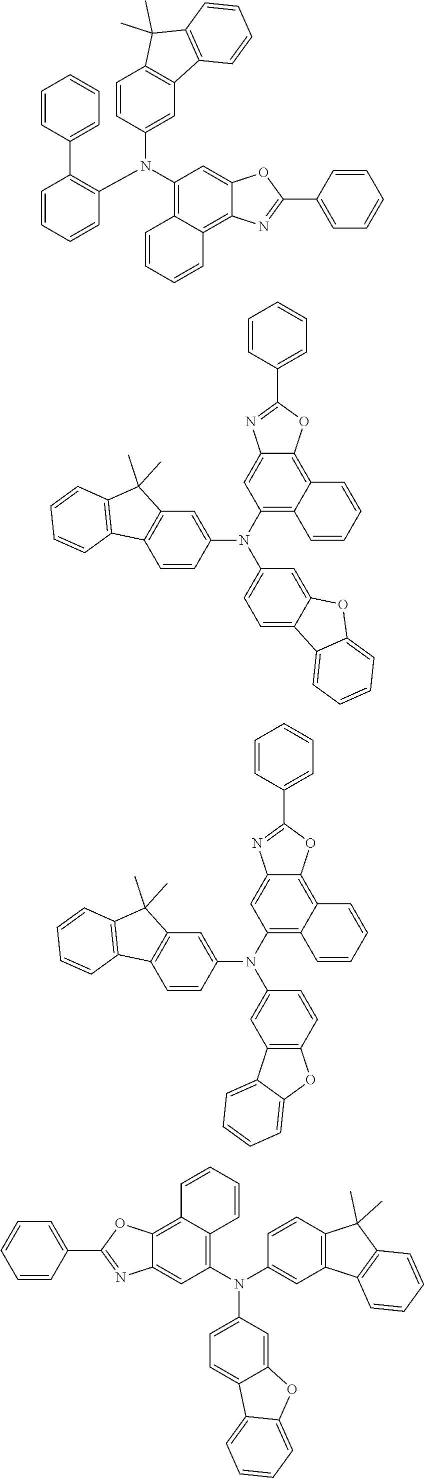

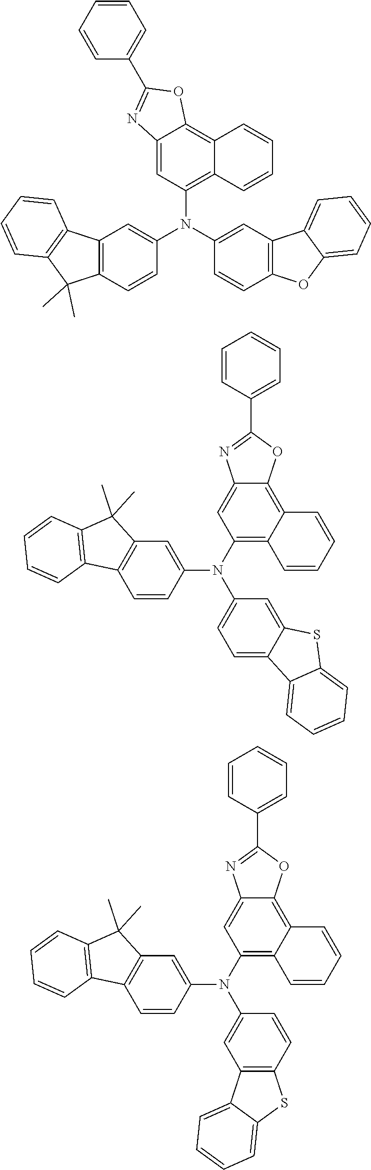

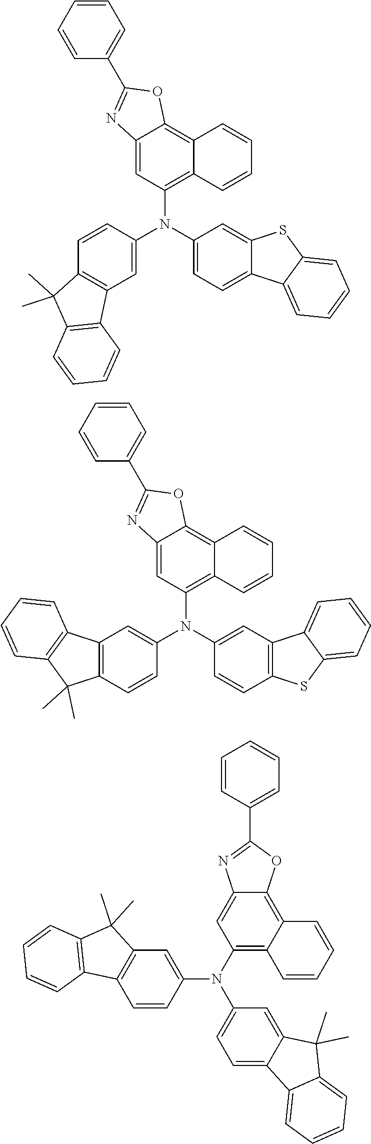

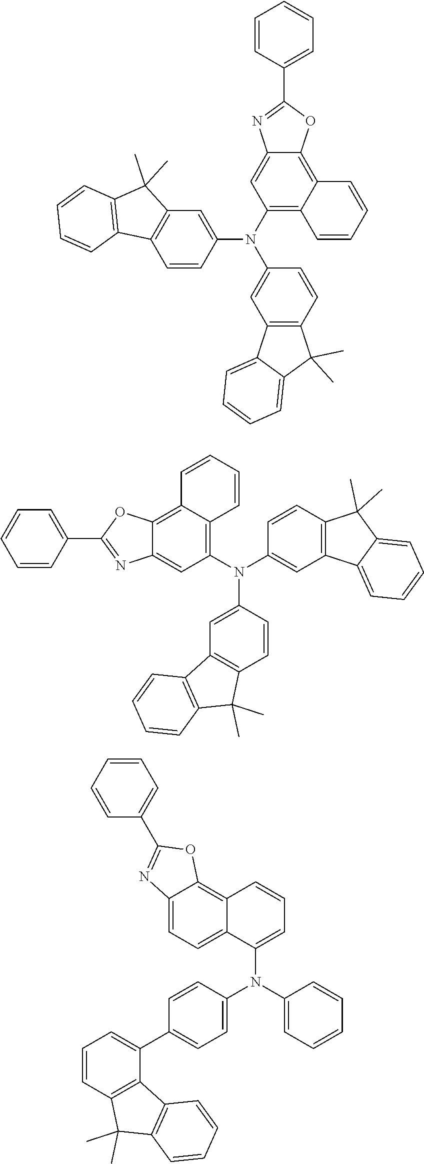

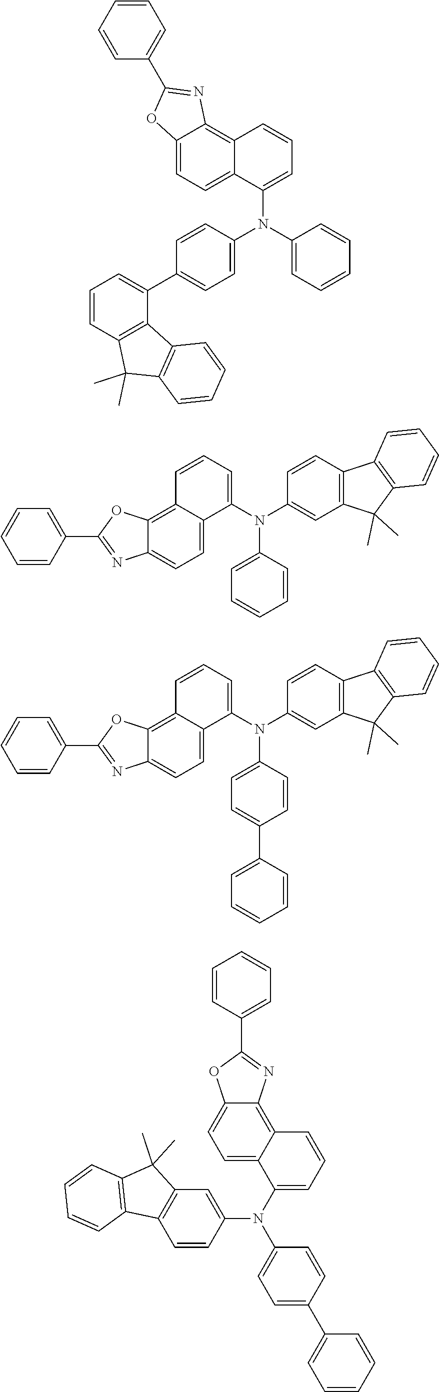

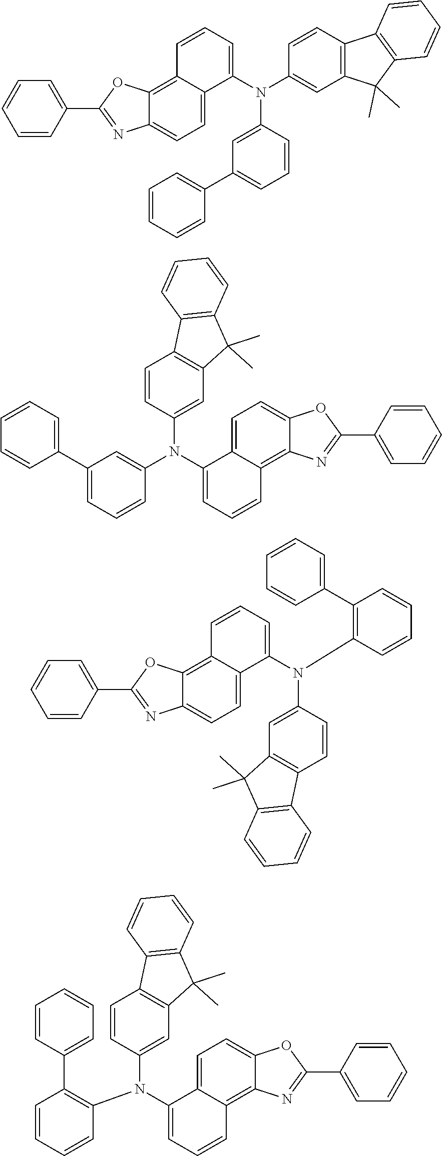

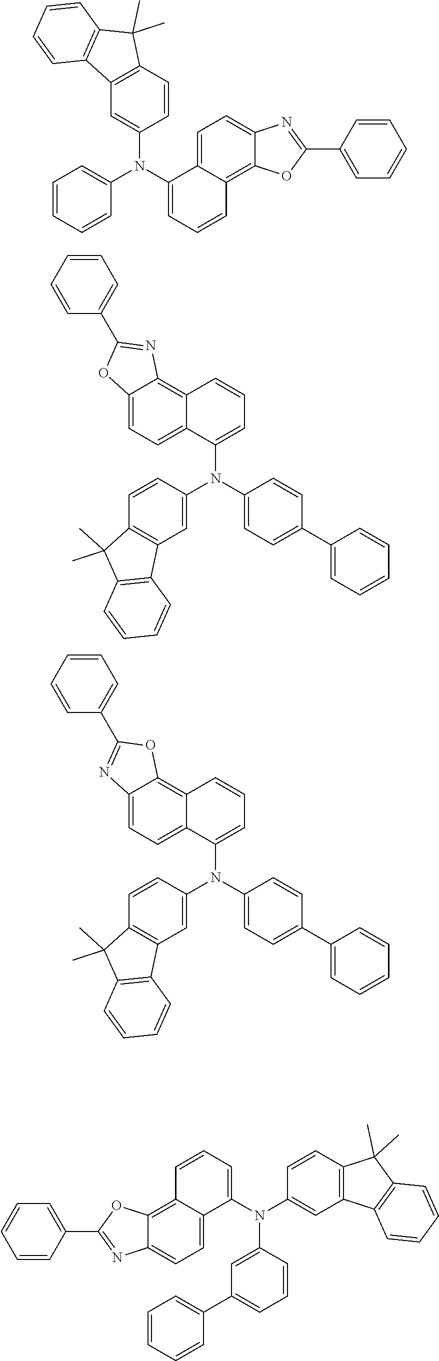

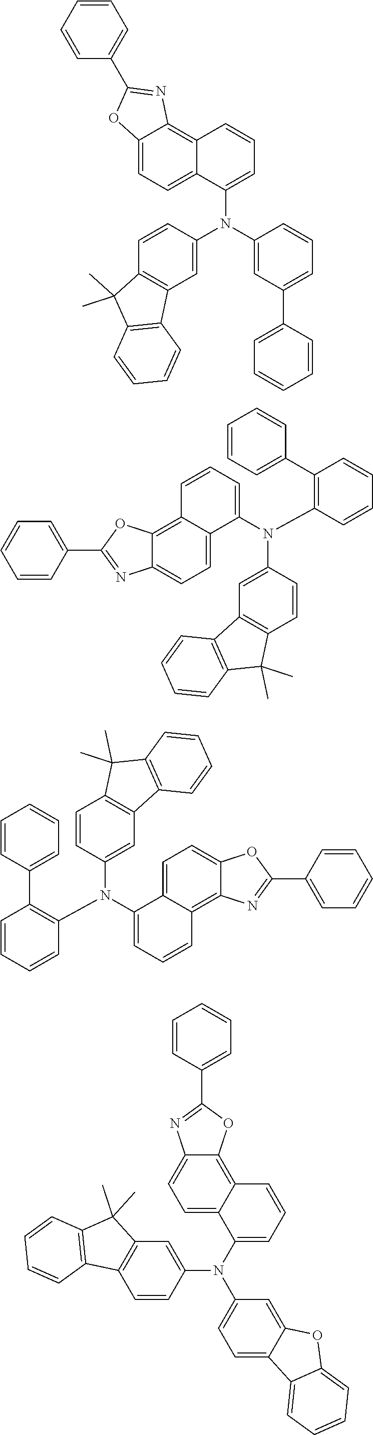

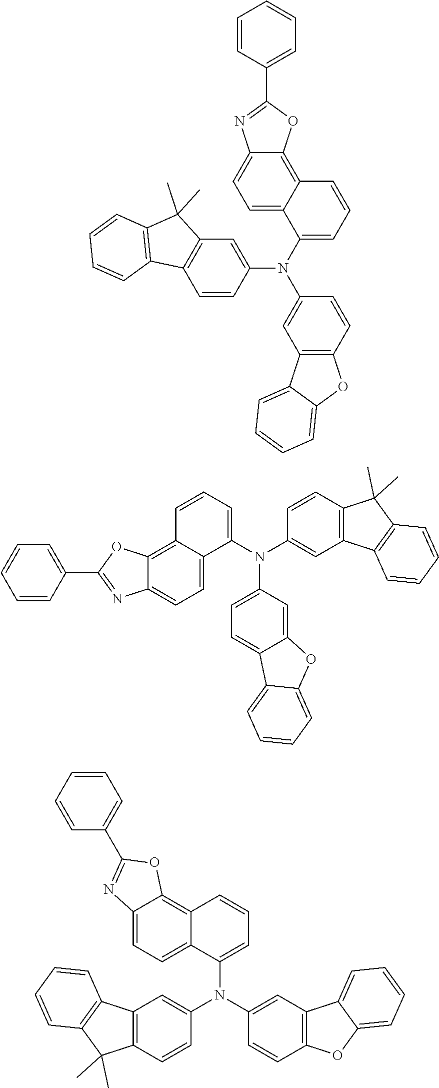 [SMILES: CC1(C)c2ccccc2-c2ccc(N(c3ccc4oc5ccccc5c4c3)c3cccc4c3ccc3nc(-c5ccccc5)oc34)cc21 ; CC1(C)c2ccccc2-c2cc(N(c3ccc4c(c3)oc3ccccc34)c3cccc4c3ccc3nc(-c5ccccc5)oc34)ccc21 ; CC1(C)c2ccccc2-c2cc(N(c3ccc4oc5ccccc5c4c3)c3cccc4c3ccc3nc(-c5ccccc5)oc34)ccc21]

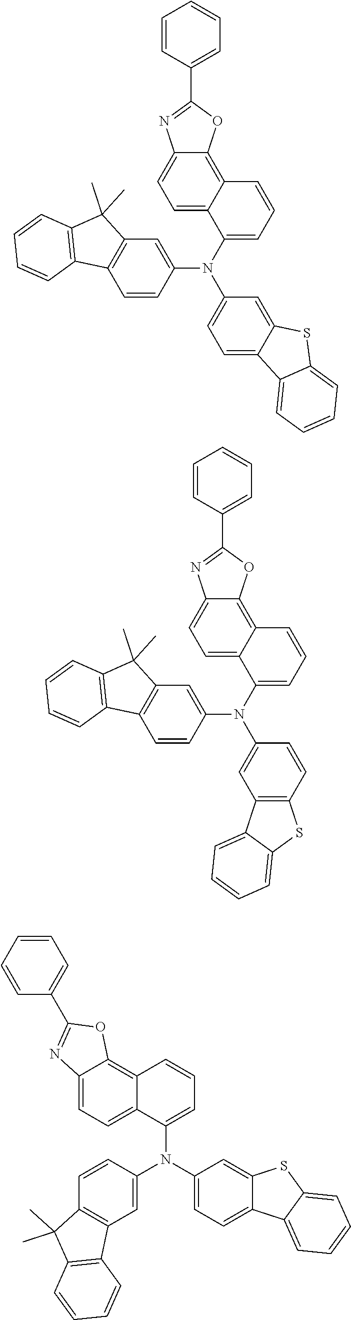 [SMILES: CC1(C)c2ccccc2-c2ccc(N(c3ccc4c(c3)sc3ccccc34)c3cccc4c3ccc3nc(-c5ccccc5)oc34)cc21 ; CC1(C)c2ccccc2-c2ccc(N(c3ccc4sc5ccccc5c4c3)c3cccc4c3ccc3nc(-c5ccccc5)oc34)cc21 ; CC1(C)c2ccccc2-c2cc(N(c3ccc4c(c3)sc3ccccc34)c3cccc4c3ccc3nc(-c5ccccc5)oc34)ccc21]

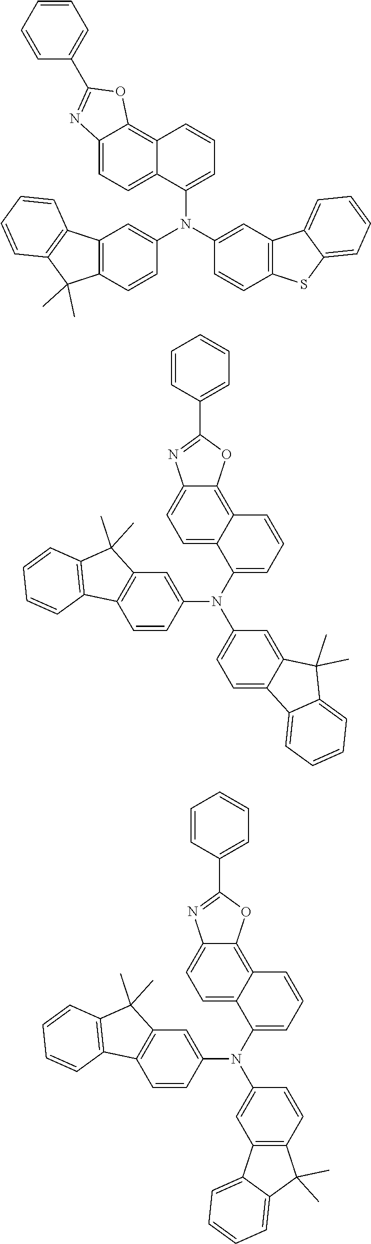 [SMILES: CC1(C)c2ccccc2-c2cc(N(c3ccc4sc5ccccc5c4c3)c3cccc4c3ccc3nc(-c5ccccc5)oc34)ccc21 ; CC1(C)c2ccccc2-c2ccc(N(c3ccc4c(c3)C(C)(C)c3ccccc3-4)c3cccc4c3ccc3nc(-c5ccccc5)oc34)cc21 ; CC1(C)c2ccccc2-c2cc(N(c3ccc4c(c3)C(C)(C)c3ccccc3-4)c3cccc4c3ccc3nc(-c5ccccc5)oc34)ccc21]

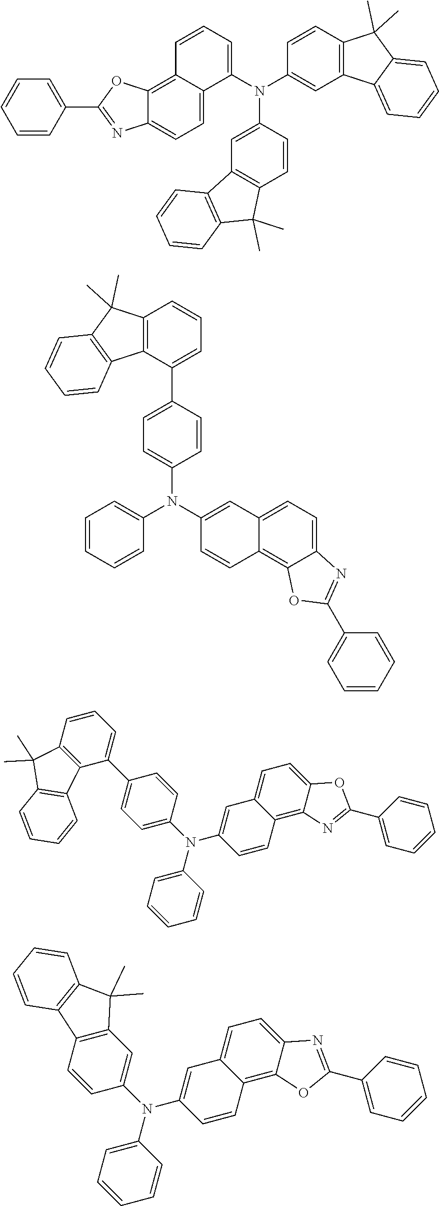 [SMILES: CC1(C)c2ccccc2-c2cc(N(c3ccc4c(c3)-c3ccccc3C4(C)C)c3cccc4c3ccc3nc(-c5ccccc5)oc34)ccc21 ; CC1(C)c2ccccc2-c2c(-c3ccc(N(c4ccccc4)c4ccc5c(ccc6nc(-c7ccccc7)oc65)c4)cc3)cccc21 ; CC1(C)c2ccccc2-c2c(-c3ccc(N(c4ccccc4)c4ccc5c(ccc6oc(-c7ccccc7)nc65)c4)cc3)cccc21 ; CC1(C)c2ccccc2-c2ccc(N(c3ccccc3)c3ccc4c(ccc5nc(-c6ccccc6)oc54)c3)cc21]

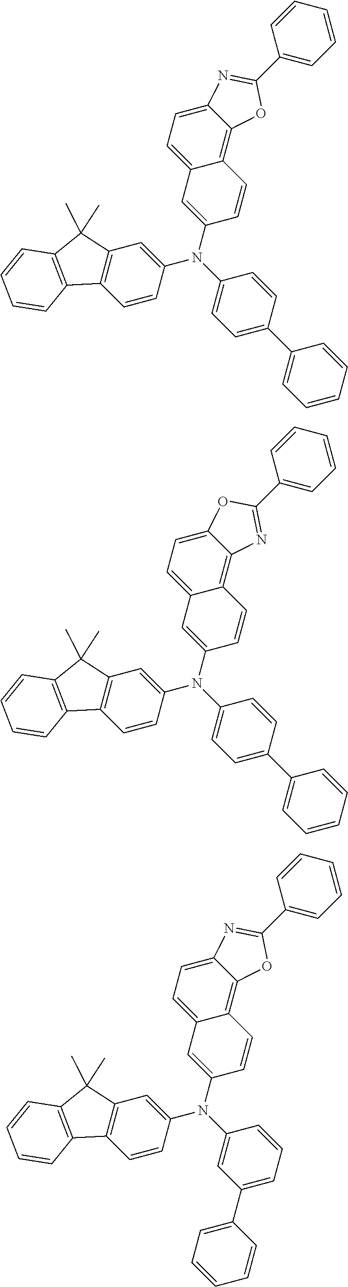

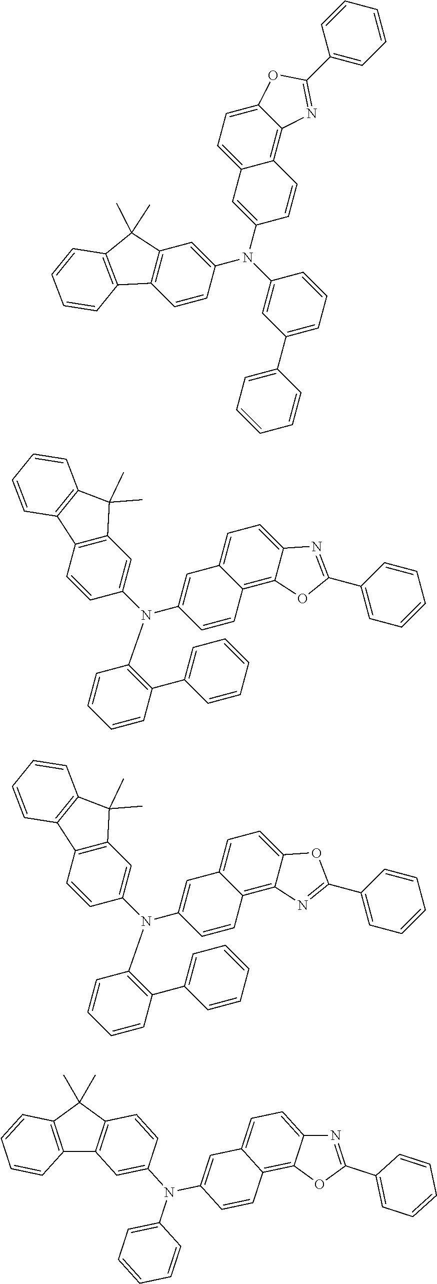

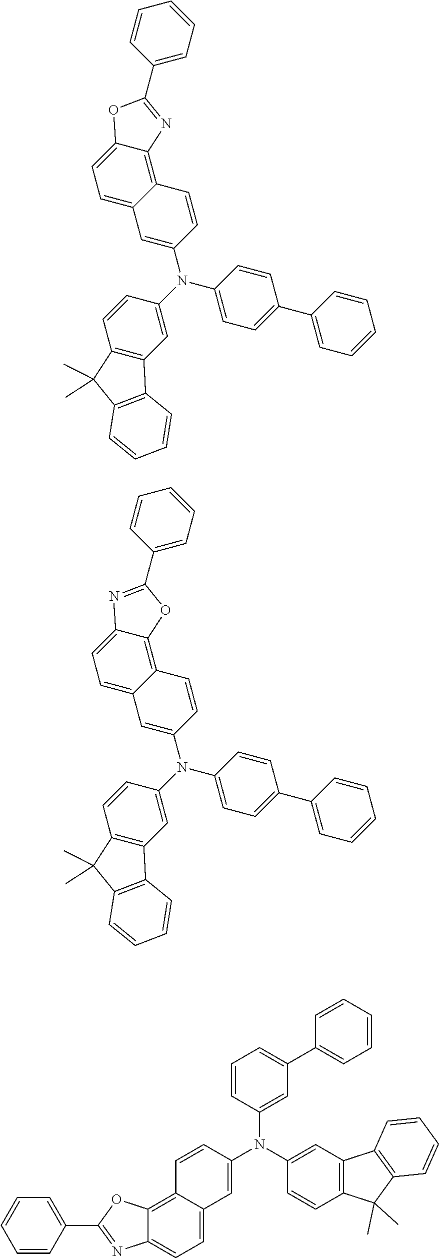

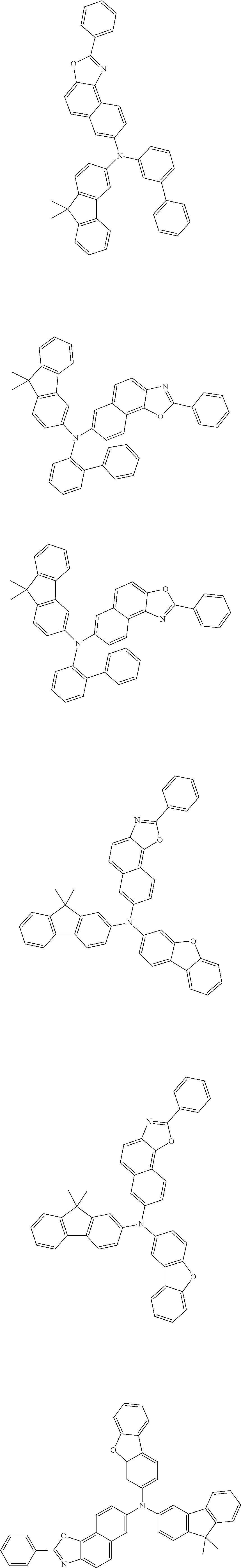 [SMILES: CC1(C)c2ccccc2-c2cc(N(c3cccc(-c4ccccc4)c3)c3ccc4c(ccc5oc(-c6ccccc6)nc54)c3)ccc21 ; CC1(C)c2ccccc2-c2cc(N(c3ccc4c(ccc5nc(-c6ccccc6)oc54)c3)c3ccccc3-c3ccccc3)ccc21 ; CC1(C)c2ccccc2-c2cc(N(c3ccc4c(ccc5oc(-c6ccccc6)nc54)c3)c3ccccc3-c3ccccc3)ccc21 ; CC1(C)c2ccccc2-c2ccc(N(c3ccc4c(ccc5nc(-c6ccccc6)oc54)c3)c3ccc4c(c3)oc3ccccc34)cc21 ; CC1(C)c2ccccc2-c2ccc(N(c3ccc4c(ccc5nc(-c6ccccc6)oc54)c3)c3ccc4oc5ccccc5c4c3)cc21 ; CC1(C)c2ccccc2-c2cc(N(c3ccc4c(ccc5nc(-c6ccccc6)oc54)c3)c3ccc4c(c3)oc3ccccc34)ccc21]

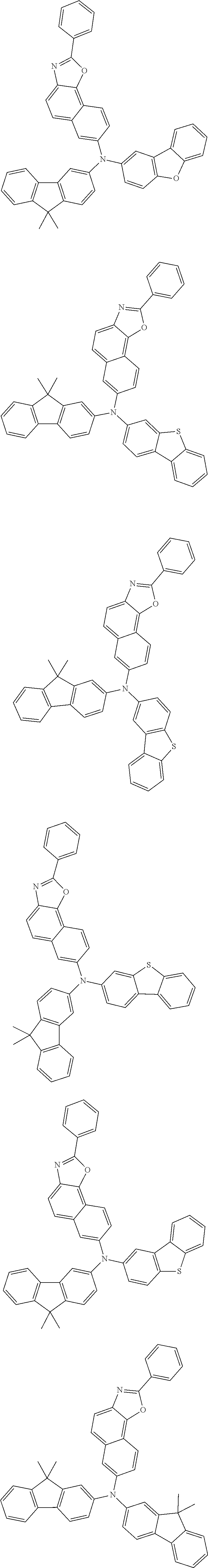

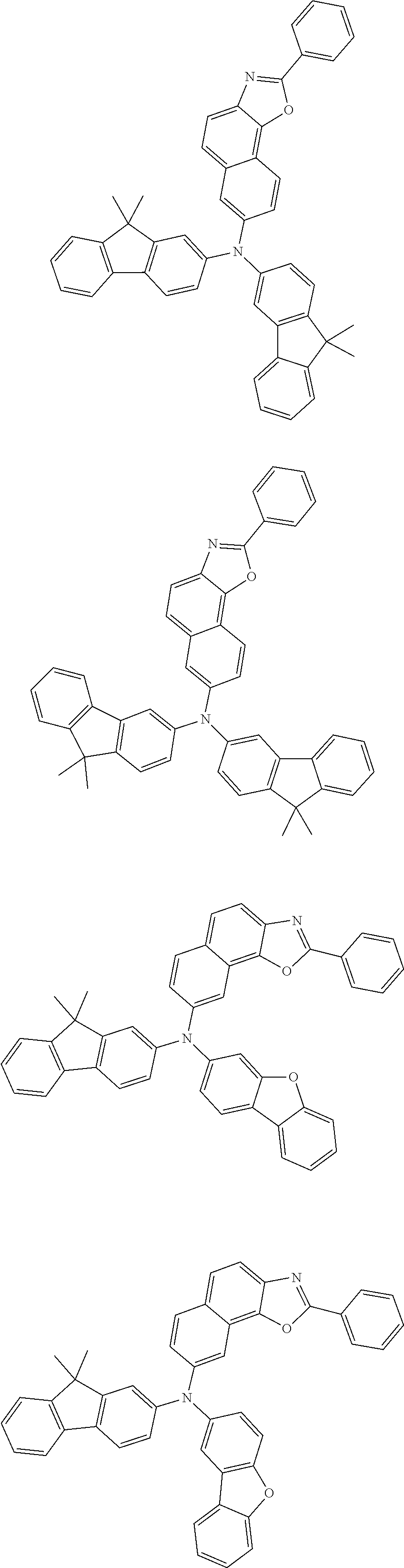 [SMILES: CC1(C)c2ccccc2-c2cc(N(c3ccc4c(c3)C(C)(C)c3ccccc3-4)c3ccc4c(ccc5nc(-c6ccccc6)oc54)c3)ccc21 ; CC1(C)c2ccccc2-c2cc(N(c3ccc4c(c3)-c3ccccc3C4(C)C)c3ccc4c(ccc5nc(-c6ccccc6)oc54)c3)ccc21 ; CC1(C)c2ccccc2-c2ccc(N(c3ccc4c(c3)oc3ccccc34)c3ccc4ccc5nc(-c6ccccc6)oc5c4c3)cc21 ; CC1(C)c2ccccc2-c2ccc(N(c3ccc4oc5ccccc5c4c3)c3ccc4ccc5nc(-c6ccccc6)oc5c4c3)cc21]

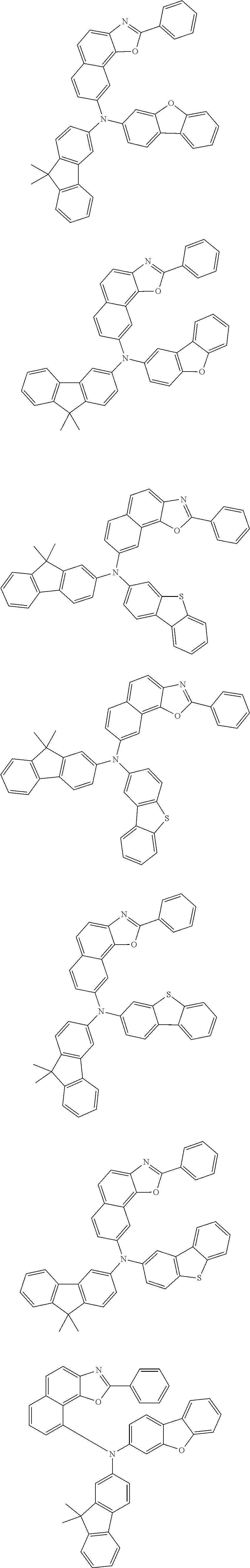

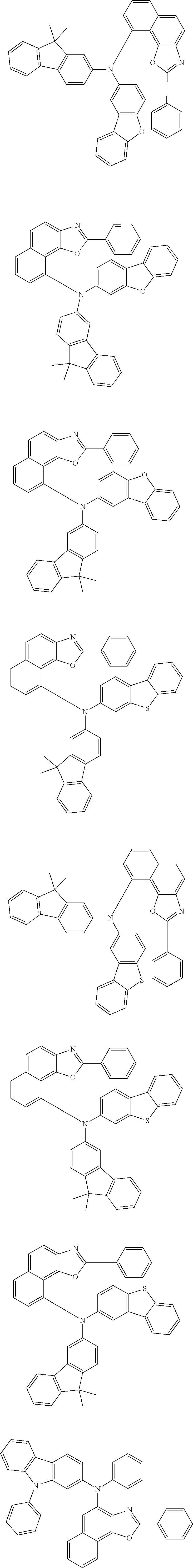

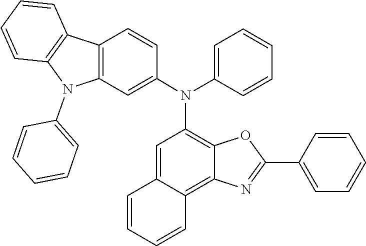 [SMILES: c1ccc(-c2nc3c(o2)c(N(c2ccccc2)c2ccc4c5ccccc5n(-c5ccccc5)c4c2)cc2ccccc23)cc1]

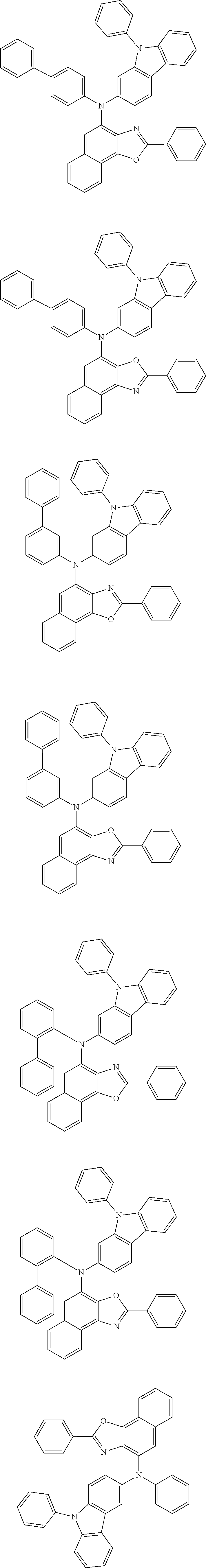

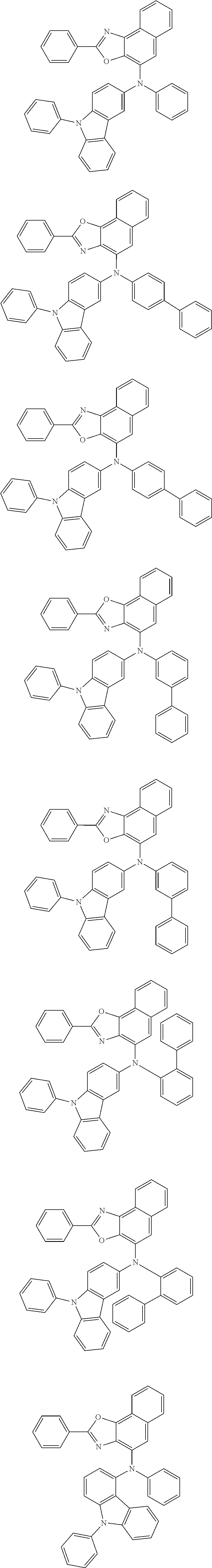

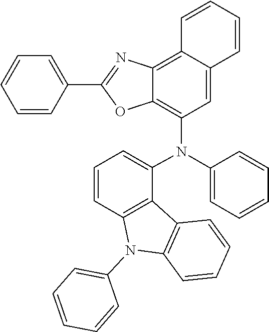 [SMILES: c1ccc(-c2nc3c(o2)c(N(c2ccccc2)c2cccc4c2c2ccccc2n4-c2ccccc2)cc2ccccc23)cc1]

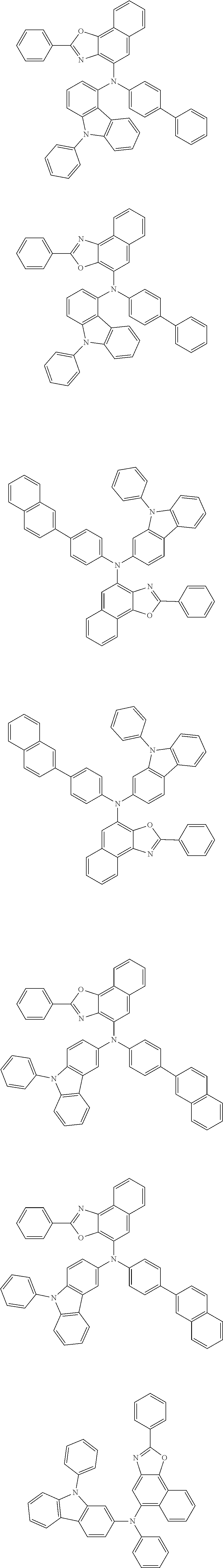

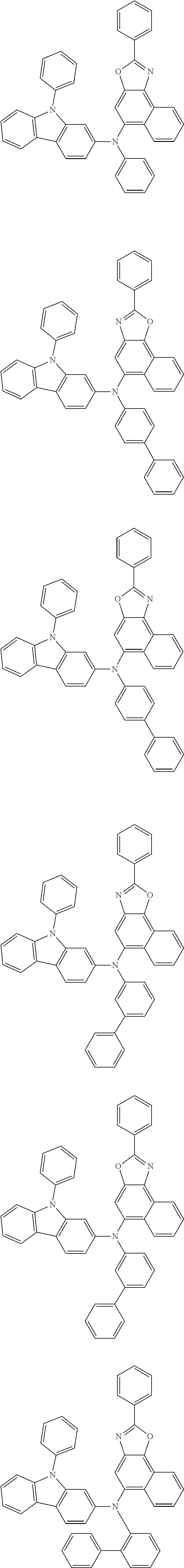

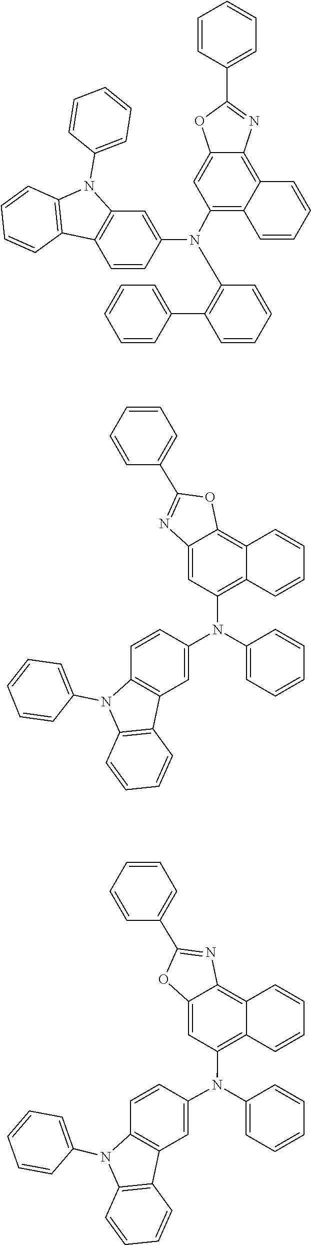 [SMILES: c1ccc(-c2nc3c(cc(N(c4ccc5c6ccccc6n(-c6ccccc6)c5c4)c4ccccc4-c4ccccc4)c4ccccc43)o2)cc1 ; c1ccc(-c2nc3cc(N(c4ccccc4)c4ccc5c(c4)c4ccccc4n5-c4ccccc4)c4ccccc4c3o2)cc1 ; c1ccc(-c2nc3c(cc(N(c4ccccc4)c4ccc5c(c4)c4ccccc4n5-c4ccccc4)c4ccccc43)o2)cc1]

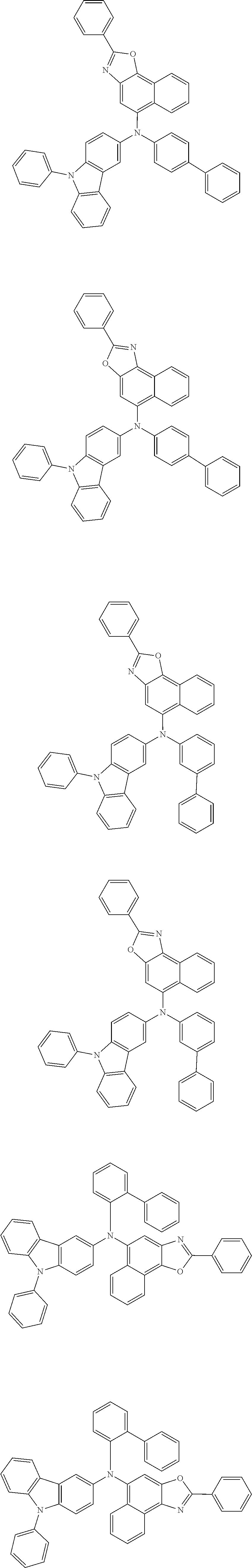

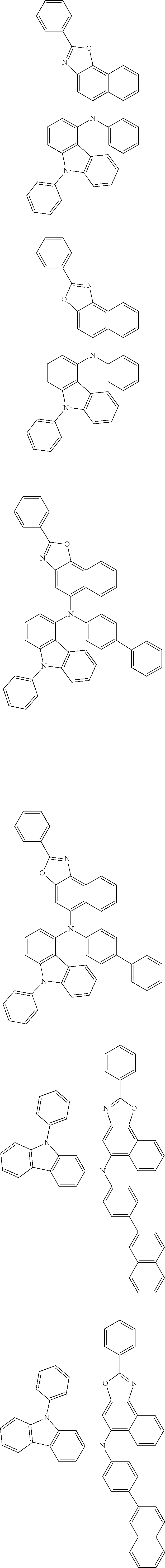

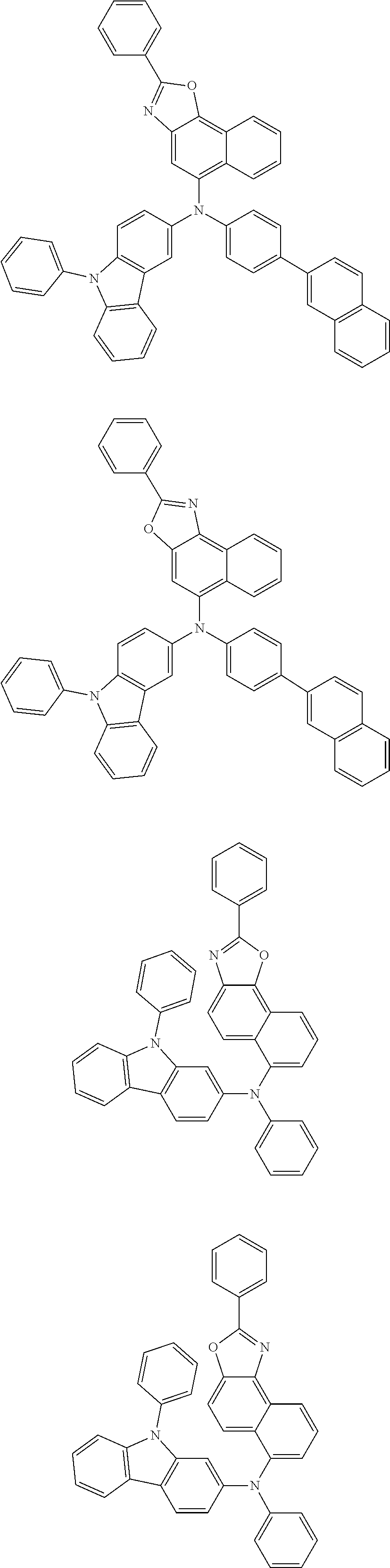 [SMILES: c1ccc(-c2nc3cc(N(c4ccc(-c5ccc6ccccc6c5)cc4)c4ccc5c(c4)c4ccccc4n5-c4ccccc4)c4ccccc4c3o2)cc1 ; c1ccc(-c2nc3c(cc(N(c4ccc(-c5ccc6ccccc6c5)cc4)c4ccc5c(c4)c4ccccc4n5-c4ccccc4)c4ccccc43)o2)cc1 ; c1ccc(-c2nc3ccc4c(N(c5ccccc5)c5ccc6c7ccccc7n(-c7ccccc7)c6c5)cccc4c3o2)cc1 ; c1ccc(-c2nc3c(ccc4c(N(c5ccccc5)c5ccc6c7ccccc7n(-c7ccccc7)c6c5)cccc43)o2)cc1]

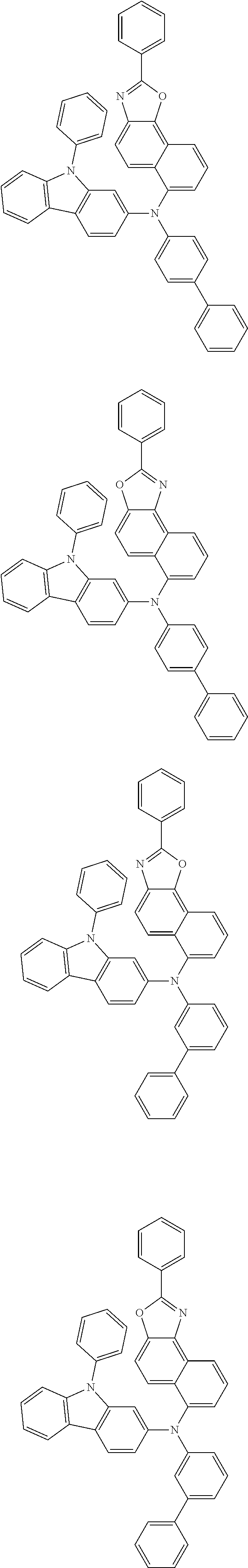 [SMILES: c1ccc(-c2ccc(N(c3ccc4c5ccccc5n(-c5ccccc5)c4c3)c3cccc4c3ccc3nc(-c5ccccc5)oc34)cc2)cc1 ; c1ccc(-c2ccc(N(c3ccc4c5ccccc5n(-c5ccccc5)c4c3)c3cccc4c3ccc3oc(-c5ccccc5)nc34)cc2)cc1 ; c1ccc(-c2cccc(N(c3ccc4c5ccccc5n(-c5ccccc5)c4c3)c3cccc4c3ccc3nc(-c5ccccc5)oc34)c2)cc1 ; c1ccc(-c2cccc(N(c3ccc4c5ccccc5n(-c5ccccc5)c4c3)c3cccc4c3ccc3oc(-c5ccccc5)nc34)c2)cc1]

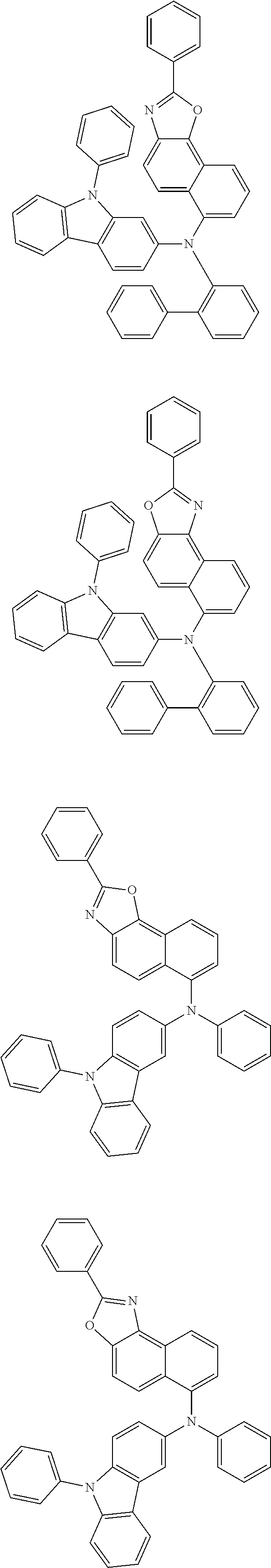 [SMILES: c1ccc(-c2nc3ccc4c(N(c5ccc6c7ccccc7n(-c7ccccc7)c6c5)c5ccccc5-c5ccccc5)cccc4c3o2)cc1 ; c1ccc(-c2nc3c(ccc4c(N(c5ccc6c7ccccc7n(-c7ccccc7)c6c5)c5ccccc5-c5ccccc5)cccc43)o2)cc1 ; c1ccc(-c2nc3ccc4c(N(c5ccccc5)c5ccc6c(c5)c5ccccc5n6-c5ccccc5)cccc4c3o2)cc1 ; c1ccc(-c2nc3c(ccc4c(N(c5ccccc5)c5ccc6c(c5)c5ccccc5n6-c5ccccc5)cccc43)o2)cc1]

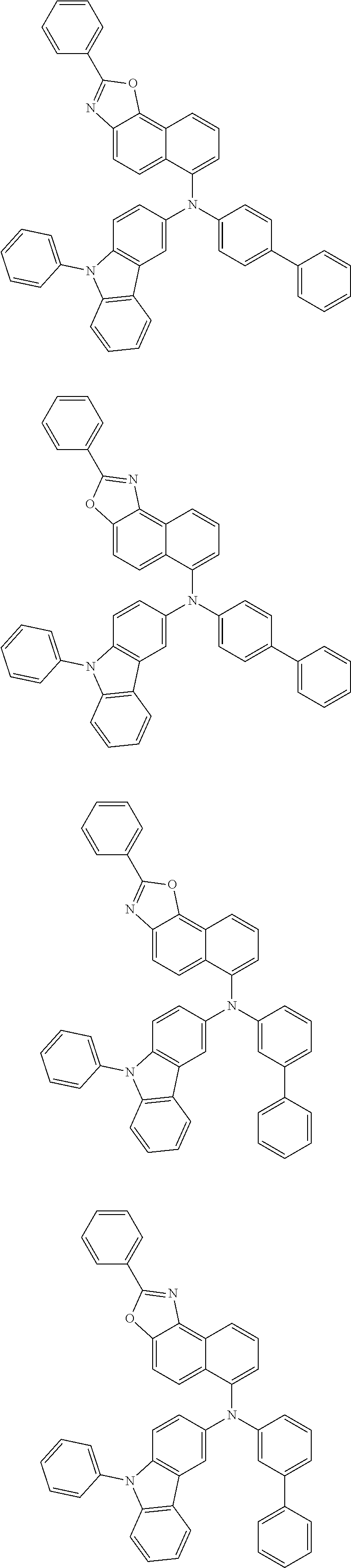 [SMILES: c1ccc(-c2ccc(N(c3ccc4c(c3)c3ccccc3n4-c3ccccc3)c3cccc4c3ccc3nc(-c5ccccc5)oc34)cc2)cc1 ; c1ccc(-c2ccc(N(c3ccc4c(c3)c3ccccc3n4-c3ccccc3)c3cccc4c3ccc3oc(-c5ccccc5)nc34)cc2)cc1 ; c1ccc(-c2cccc(N(c3ccc4c(c3)c3ccccc3n4-c3ccccc3)c3cccc4c3ccc3nc(-c5ccccc5)oc34)c2)cc1 ; c1ccc(-c2cccc(N(c3ccc4c(c3)c3ccccc3n4-c3ccccc3)c3cccc4c3ccc3oc(-c5ccccc5)nc34)c2)cc1]

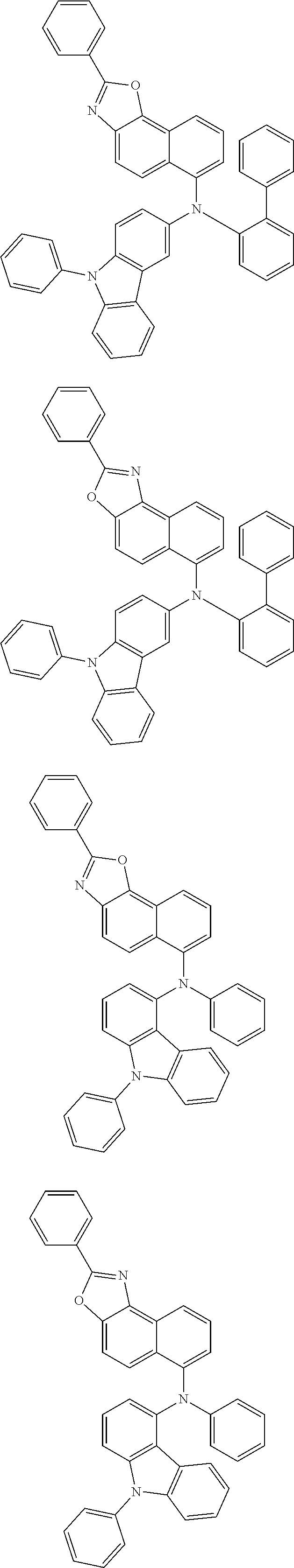

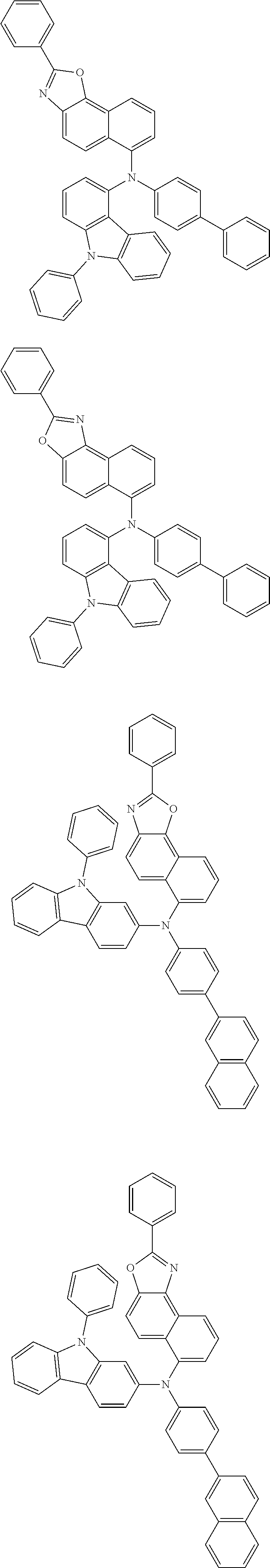

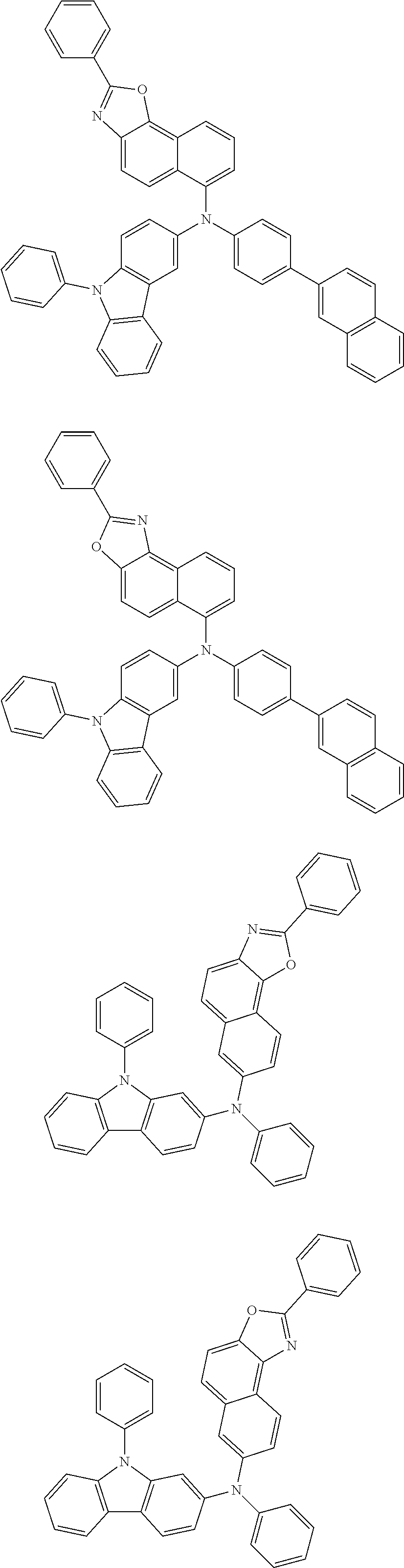 [SMILES: c1ccc(-c2nc3ccc4c(N(c5ccc(-c6ccc7ccccc7c6)cc5)c5ccc6c(c5)c5ccccc5n6-c5ccccc5)cccc4c3o2)cc1 ; c1ccc(-c2nc3c(ccc4c(N(c5ccc(-c6ccc7ccccc7c6)cc5)c5ccc6c(c5)c5ccccc5n6-c5ccccc5)cccc43)o2)cc1 ; c1ccc(-c2nc3ccc4cc(N(c5ccccc5)c5ccc6c7ccccc7n(-c7ccccc7)c6c5)ccc4c3o2)cc1 ; c1ccc(-c2nc3c(ccc4cc(N(c5ccccc5)c5ccc6c7ccccc7n(-c7ccccc7)c6c5)ccc43)o2)cc1]

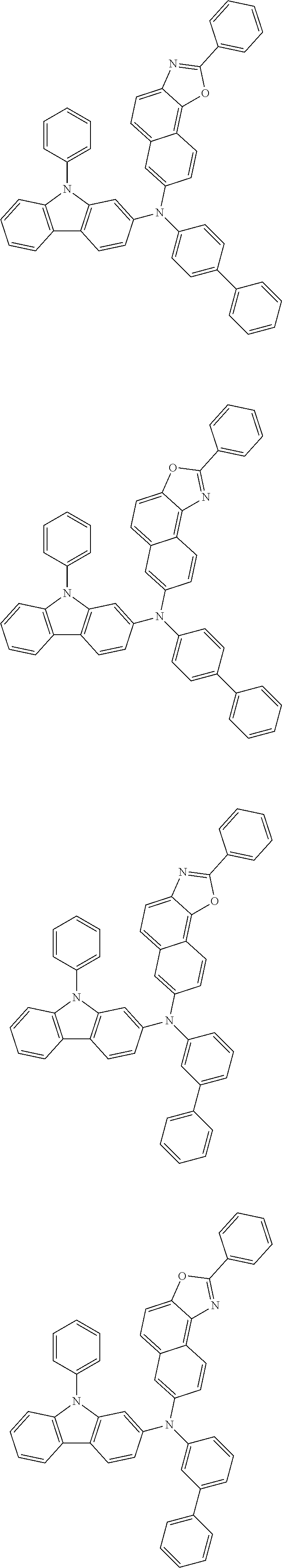 [SMILES: c1ccc(-c2ccc(N(c3ccc4c(ccc5nc(-c6ccccc6)oc54)c3)c3ccc4c5ccccc5n(-c5ccccc5)c4c3)cc2)cc1 ; c1ccc(-c2ccc(N(c3ccc4c(ccc5oc(-c6ccccc6)nc54)c3)c3ccc4c5ccccc5n(-c5ccccc5)c4c3)cc2)cc1 ; c1ccc(-c2cccc(N(c3ccc4c(ccc5nc(-c6ccccc6)oc54)c3)c3ccc4c5ccccc5n(-c5ccccc5)c4c3)c2)cc1 ; c1ccc(-c2cccc(N(c3ccc4c(ccc5oc(-c6ccccc6)nc54)c3)c3ccc4c5ccccc5n(-c5ccccc5)c4c3)c2)cc1]

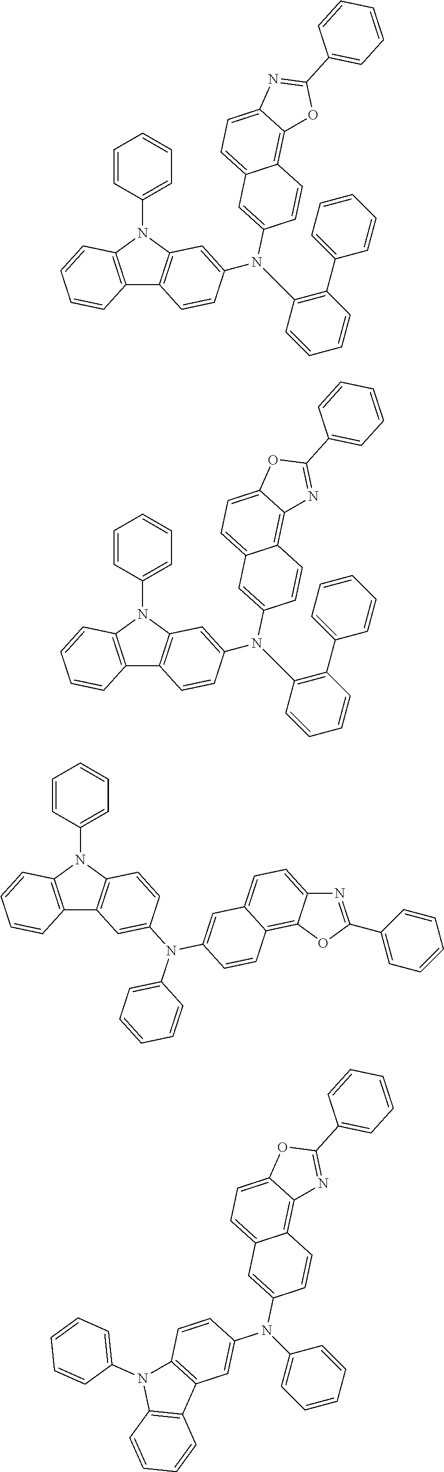

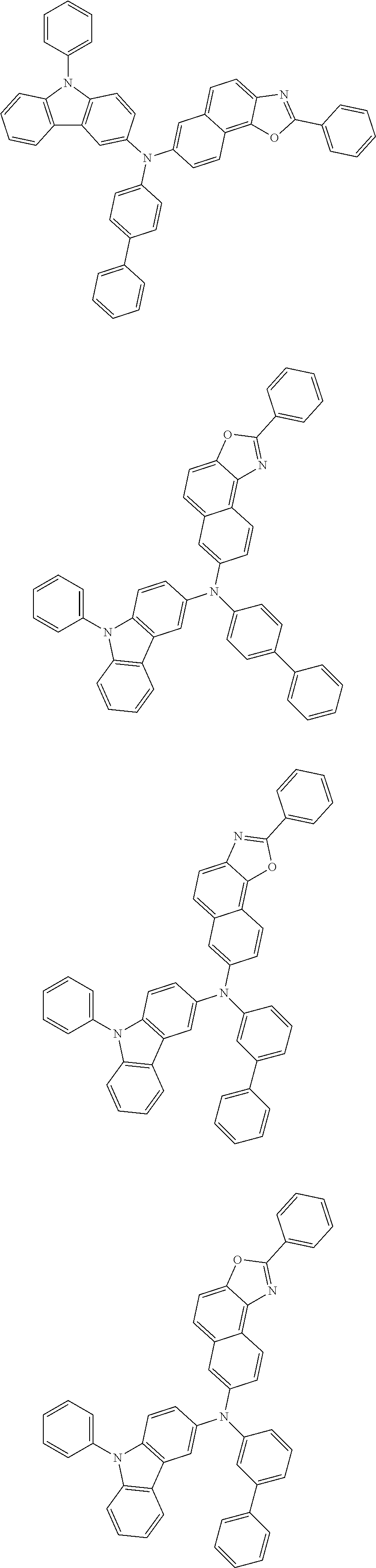 [SMILES: c1ccc(-c2ccc(N(c3ccc4c(ccc5nc(-c6ccccc6)oc54)c3)c3ccc4c(c3)c3ccccc3n4-c3ccccc3)cc2)cc1 ; c1ccc(-c2ccc(N(c3ccc4c(ccc5oc(-c6ccccc6)nc54)c3)c3ccc4c(c3)c3ccccc3n4-c3ccccc3)cc2)cc1 ; c1ccc(-c2cccc(N(c3ccc4c(ccc5nc(-c6ccccc6)oc54)c3)c3ccc4c(c3)c3ccccc3n4-c3ccccc3)c2)cc1 ; c1ccc(-c2cccc(N(c3ccc4c(ccc5oc(-c6ccccc6)nc54)c3)c3ccc4c(c3)c3ccccc3n4-c3ccccc3)c2)cc1]

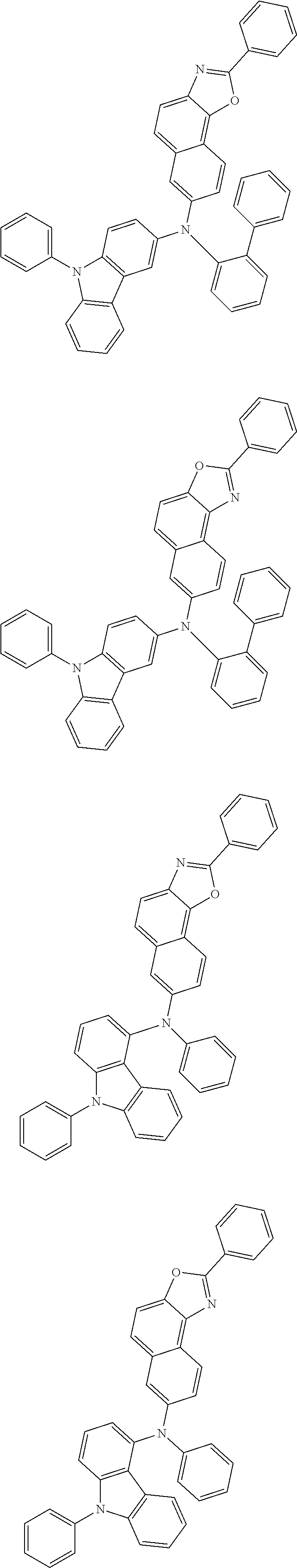 [SMILES: c1ccc(-c2nc3ccc4cc(N(c5ccc6c(c5)c5ccccc5n6-c5ccccc5)c5ccccc5-c5ccccc5)ccc4c3o2)cc1 ; c1ccc(-c2nc3c(ccc4cc(N(c5ccc6c(c5)c5ccccc5n6-c5ccccc5)c5ccccc5-c5ccccc5)ccc43)o2)cc1 ; c1ccc(-c2nc3ccc4cc(N(c5ccccc5)c5cccc6c5c5ccccc5n6-c5ccccc5)ccc4c3o2)cc1 ; c1ccc(-c2nc3c(ccc4cc(N(c5ccccc5)c5cccc6c5c5ccccc5n6-c5ccccc5)ccc43)o2)cc1]

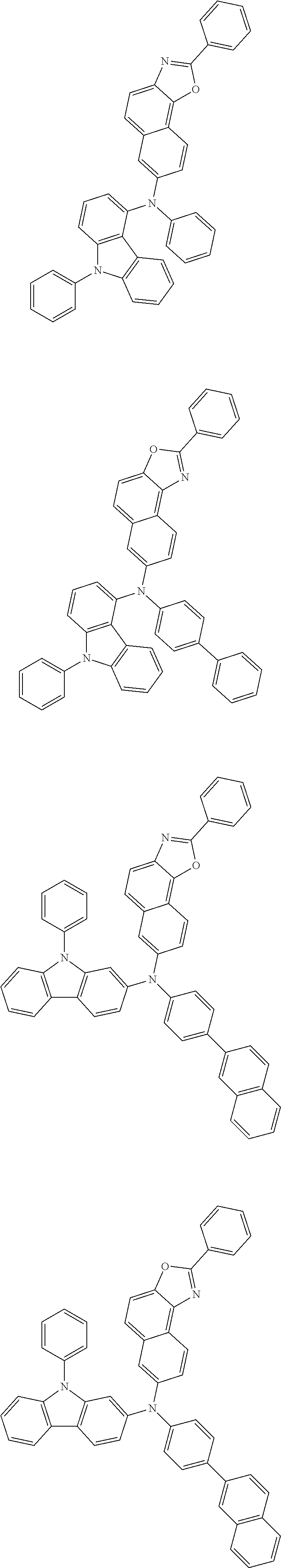 [SMILES: c1ccc(-c2nc3ccc4cc(N(c5ccccc5)c5cccc6c5c5ccccc5n6-c5ccccc5)ccc4c3o2)cc1 ; c1ccc(-c2ccc(N(c3ccc4c(ccc5oc(-c6ccccc6)nc54)c3)c3cccc4c3c3ccccc3n4-c3ccccc3)cc2)cc1 ; c1ccc(-c2nc3ccc4cc(N(c5ccc(-c6ccc7ccccc7c6)cc5)c5ccc6c7ccccc7n(-c7ccccc7)c6c5)ccc4c3o2)cc1 ; c1ccc(-c2nc3c(ccc4cc(N(c5ccc(-c6ccc7ccccc7c6)cc5)c5ccc6c7ccccc7n(-c7ccccc7)c6c5)ccc43)o2)cc1]

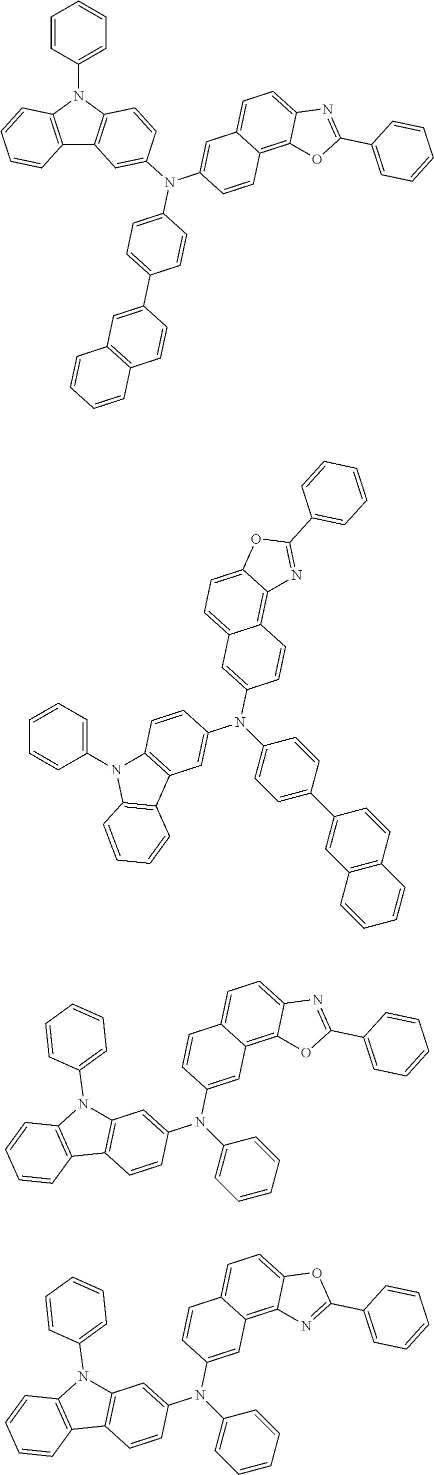 [SMILES: c1ccc(-c2nc3ccc4cc(N(c5ccc(-c6ccc7ccccc7c6)cc5)c5ccc6c(c5)c5ccccc5n6-c5ccccc5)ccc4c3o2)cc1 ; c1ccc(-c2nc3c(ccc4cc(N(c5ccc(-c6ccc7ccccc7c6)cc5)c5ccc6c(c5)c5ccccc5n6-c5ccccc5)ccc43)o2)cc1 ; c1ccc(-c2nc3ccc4ccc(N(c5ccccc5)c5ccc6c7ccccc7n(-c7ccccc7)c6c5)cc4c3o2)cc1 ; c1ccc(-c2nc3c(ccc4ccc(N(c5ccccc5)c5ccc6c7ccccc7n(-c7ccccc7)c6c5)cc43)o2)cc1]

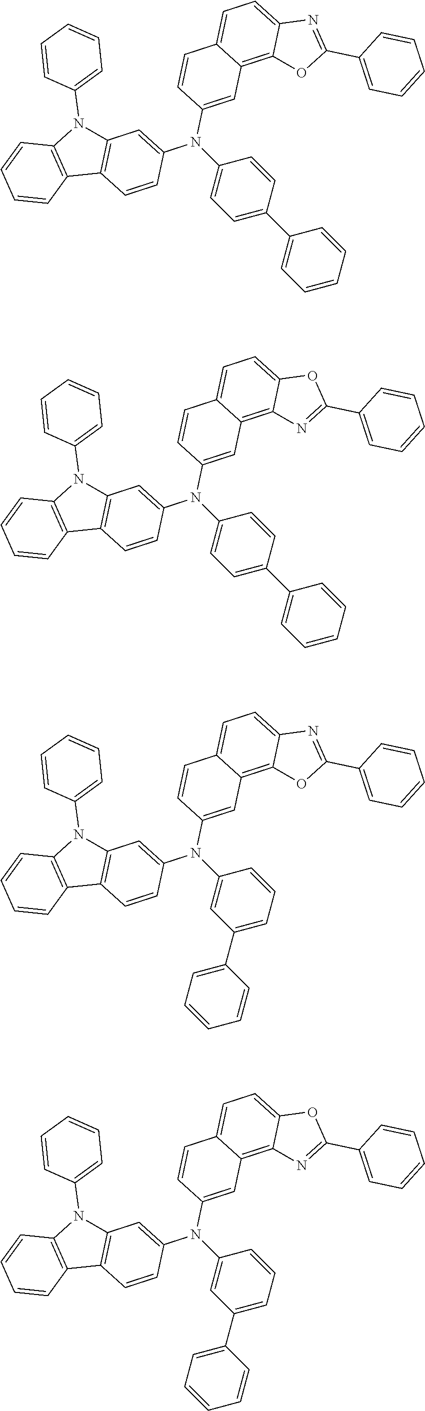 [SMILES: c1ccc(-c2ccc(N(c3ccc4ccc5nc(-c6ccccc6)oc5c4c3)c3ccc4c5ccccc5n(-c5ccccc5)c4c3)cc2)cc1 ; c1ccc(-c2ccc(N(c3ccc4ccc5oc(-c6ccccc6)nc5c4c3)c3ccc4c5ccccc5n(-c5ccccc5)c4c3)cc2)cc1 ; c1ccc(-c2cccc(N(c3ccc4ccc5nc(-c6ccccc6)oc5c4c3)c3ccc4c5ccccc5n(-c5ccccc5)c4c3)c2)cc1 ; c1ccc(-c2cccc(N(c3ccc4ccc5oc(-c6ccccc6)nc5c4c3)c3ccc4c5ccccc5n(-c5ccccc5)c4c3)c2)cc1]

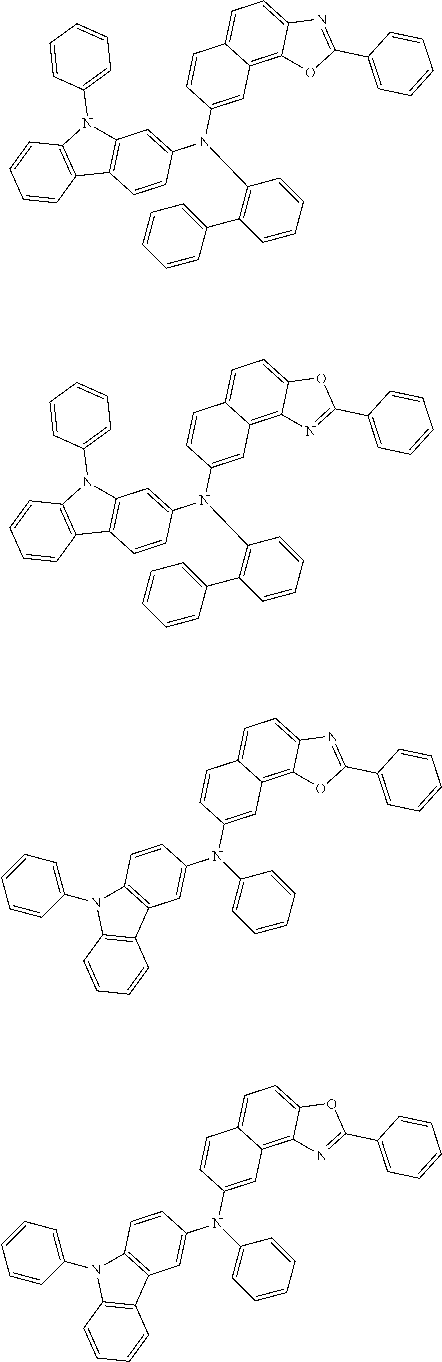 [SMILES: c1ccc(-c2nc3ccc4ccc(N(c5ccc6c7ccccc7n(-c7ccccc7)c6c5)c5ccccc5-c5ccccc5)cc4c3o2)cc1 ; c1ccc(-c2nc3c(ccc4ccc(N(c5ccc6c7ccccc7n(-c7ccccc7)c6c5)c5ccccc5-c5ccccc5)cc43)o2)cc1 ; c1ccc(-c2nc3ccc4ccc(N(c5ccccc5)c5ccc6c(c5)c5ccccc5n6-c5ccccc5)cc4c3o2)cc1 ; c1ccc(-c2nc3c(ccc4ccc(N(c5ccccc5)c5ccc6c(c5)c5ccccc5n6-c5ccccc5)cc43)o2)cc1]

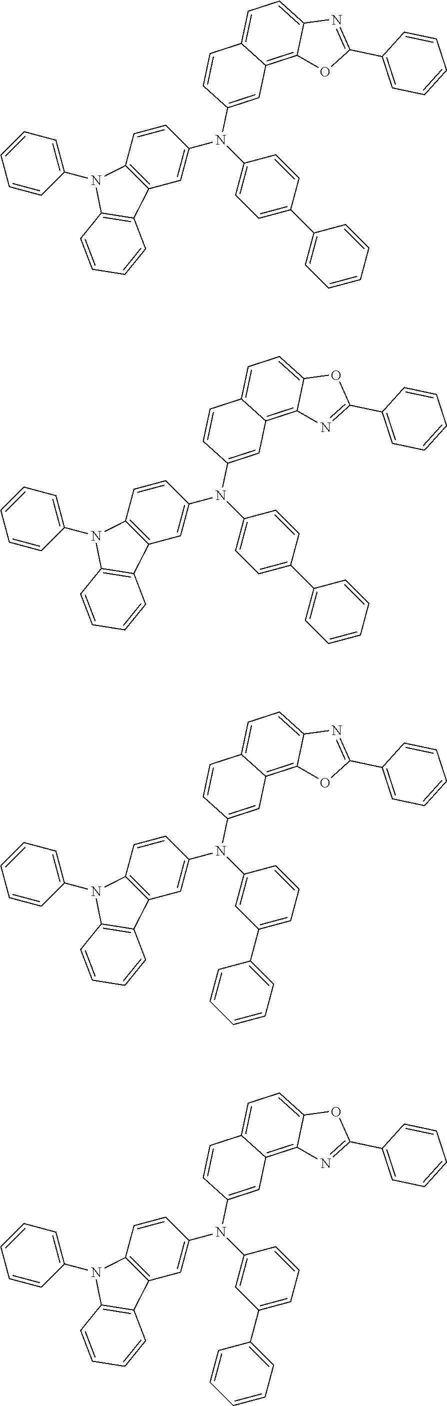 [SMILES: c1ccc(-c2ccc(N(c3ccc4ccc5nc(-c6ccccc6)oc5c4c3)c3ccc4c(c3)c3ccccc3n4-c3ccccc3)cc2)cc1 ; c1ccc(-c2ccc(N(c3ccc4ccc5oc(-c6ccccc6)nc5c4c3)c3ccc4c(c3)c3ccccc3n4-c3ccccc3)cc2)cc1 ; c1ccc(-c2cccc(N(c3ccc4ccc5nc(-c6ccccc6)oc5c4c3)c3ccc4c(c3)c3ccccc3n4-c3ccccc3)c2)cc1 ; c1ccc(-c2cccc(N(c3ccc4ccc5oc(-c6ccccc6)nc5c4c3)c3ccc4c(c3)c3ccccc3n4-c3ccccc3)c2)cc1]

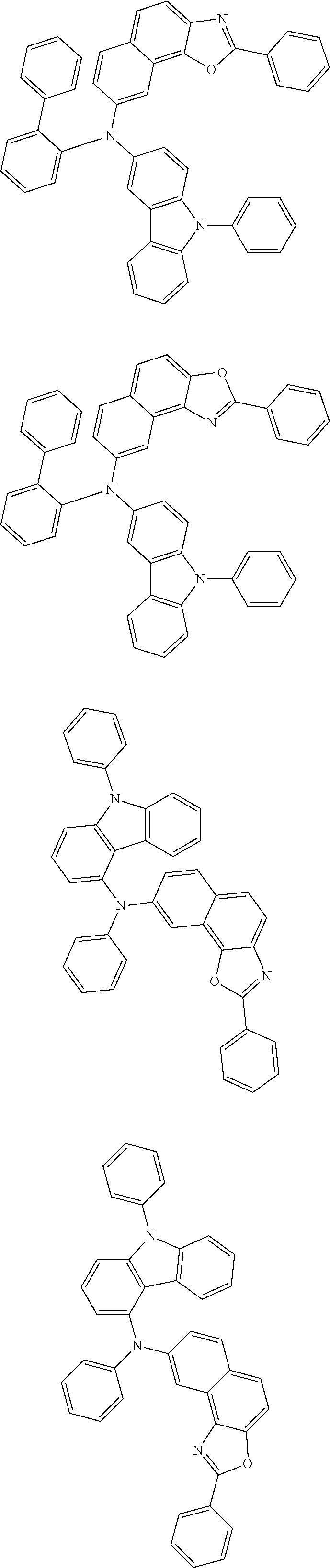 [SMILES: c1ccc(-c2nc3ccc4ccc(N(c5ccc6c(c5)c5ccccc5n6-c5ccccc5)c5ccccc5-c5ccccc5)cc4c3o2)cc1 ; c1ccc(-c2nc3c(ccc4ccc(N(c5ccc6c(c5)c5ccccc5n6-c5ccccc5)c5ccccc5-c5ccccc5)cc43)o2)cc1 ; c1ccc(-c2nc3ccc4ccc(N(c5ccccc5)c5cccc6c5c5ccccc5n6-c5ccccc5)cc4c3o2)cc1 ; c1ccc(-c2nc3c(ccc4ccc(N(c5ccccc5)c5cccc6c5c5ccccc5n6-c5ccccc5)cc43)o2)cc1]

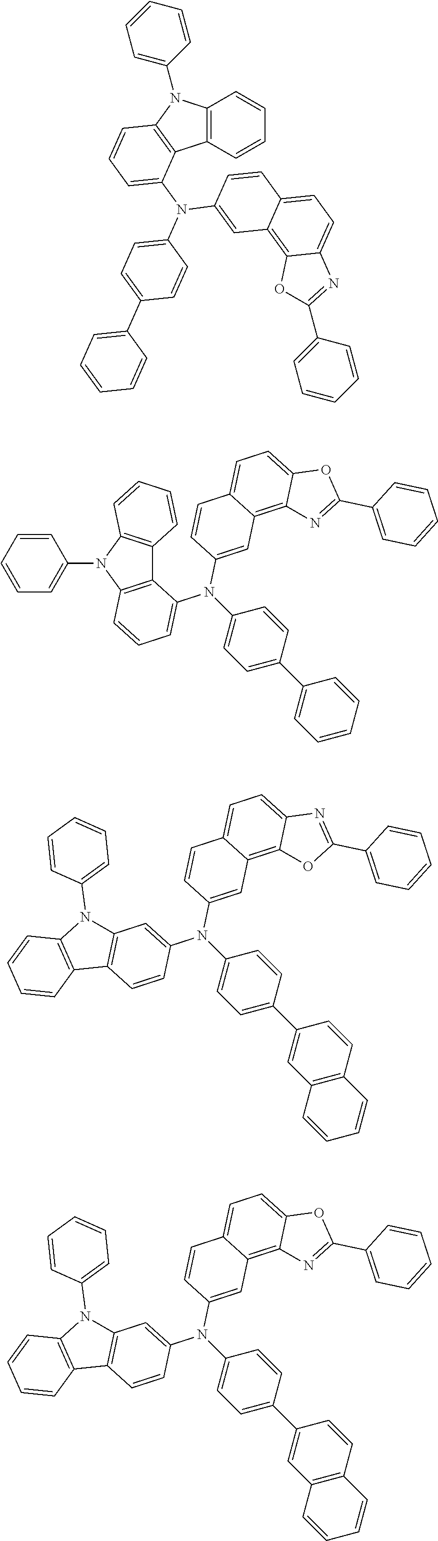 [SMILES: c1ccc(-c2ccc(N(c3ccc4ccc5nc(-c6ccccc6)oc5c4c3)c3cccc4c3c3ccccc3n4-c3ccccc3)cc2)cc1 ; c1ccc(-c2ccc(N(c3ccc4ccc5oc(-c6ccccc6)nc5c4c3)c3cccc4c3c3ccccc3n4-c3ccccc3)cc2)cc1 ; c1ccc(-c2nc3ccc4ccc(N(c5ccc(-c6ccc7ccccc7c6)cc5)c5ccc6c7ccccc7n(-c7ccccc7)c6c5)cc4c3o2)cc1 ; c1ccc(-c2nc3c(ccc4ccc(N(c5ccc(-c6ccc7ccccc7c6)cc5)c5ccc6c7ccccc7n(-c7ccccc7)c6c5)cc43)o2)cc1]

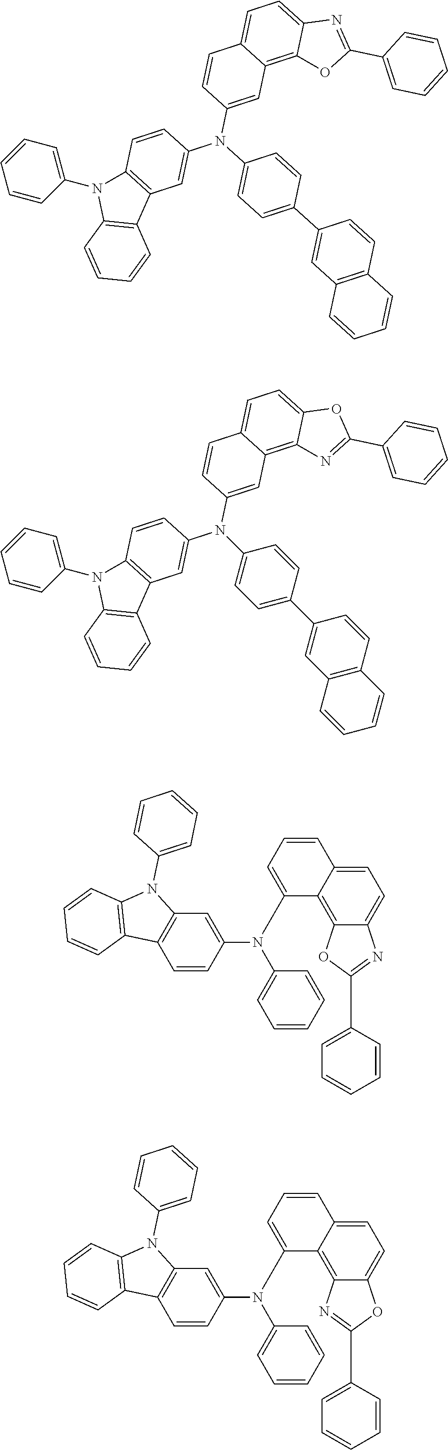 [SMILES: c1ccc(-c2nc3ccc4ccc(N(c5ccc(-c6ccc7ccccc7c6)cc5)c5ccc6c(c5)c5ccccc5n6-c5ccccc5)cc4c3o2)cc1 ; c1ccc(-c2nc3c(ccc4ccc(N(c5ccc(-c6ccc7ccccc7c6)cc5)c5ccc6c(c5)c5ccccc5n6-c5ccccc5)cc43)o2)cc1 ; c1ccc(-c2nc3ccc4cccc(N(c5ccccc5)c5ccc6c7ccccc7n(-c7ccccc7)c6c5)c4c3o2)cc1 ; c1ccc(-c2nc3c(ccc4cccc(N(c5ccccc5)c5ccc6c7ccccc7n(-c7ccccc7)c6c5)c43)o2)cc1]

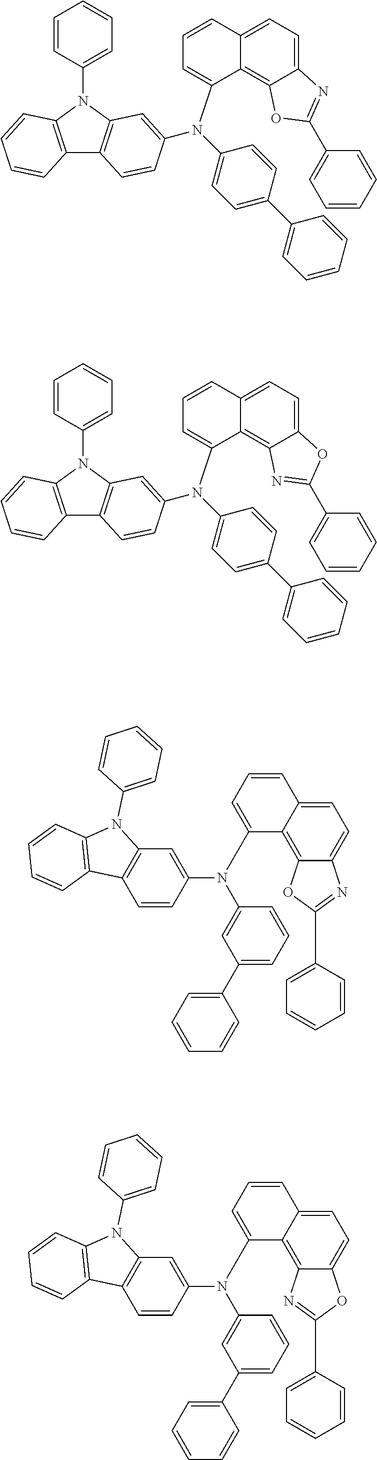 [SMILES: c1ccc(-c2ccc(N(c3ccc4c5ccccc5n(-c5ccccc5)c4c3)c3cccc4ccc5nc(-c6ccccc6)oc5c34)cc2)cc1 ; c1ccc(-c2ccc(N(c3ccc4c5ccccc5n(-c5ccccc5)c4c3)c3cccc4ccc5oc(-c6ccccc6)nc5c34)cc2)cc1 ; c1ccc(-c2cccc(N(c3ccc4c5ccccc5n(-c5ccccc5)c4c3)c3cccc4ccc5nc(-c6ccccc6)oc5c34)c2)cc1 ; c1ccc(-c2cccc(N(c3ccc4c5ccccc5n(-c5ccccc5)c4c3)c3cccc4ccc5oc(-c6ccccc6)nc5c34)c2)cc1]

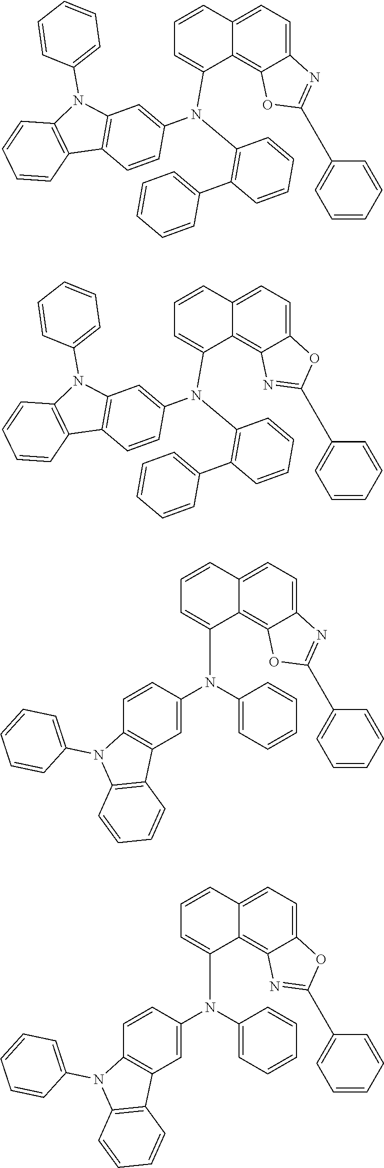 [SMILES: c1ccc(-c2nc3ccc4cccc(N(c5ccc6c7ccccc7n(-c7ccccc7)c6c5)c5ccccc5-c5ccccc5)c4c3o2)cc1 ; c1ccc(-c2nc3c(ccc4cccc(N(c5ccc6c7ccccc7n(-c7ccccc7)c6c5)c5ccccc5-c5ccccc5)c43)o2)cc1 ; c1ccc(-c2nc3ccc4cccc(N(c5ccccc5)c5ccc6c(c5)c5ccccc5n6-c5ccccc5)c4c3o2)cc1 ; c1ccc(-c2nc3c(ccc4cccc(N(c5ccccc5)c5ccc6c(c5)c5ccccc5n6-c5ccccc5)c43)o2)cc1]

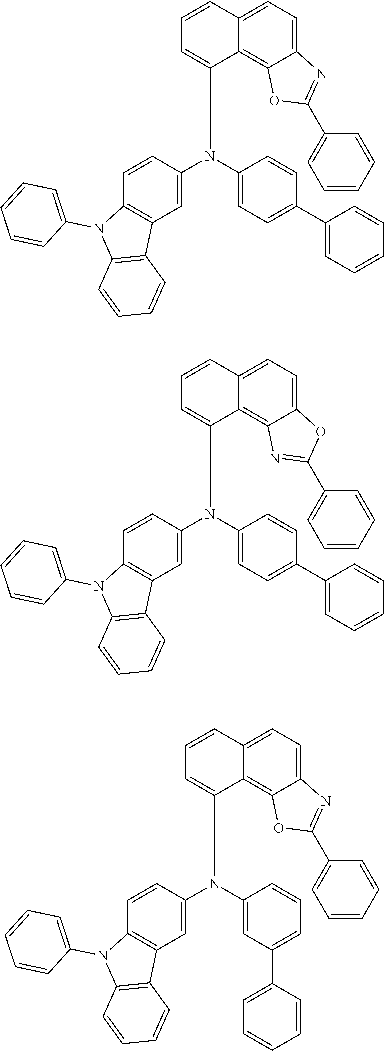 [SMILES: c1ccc(-c2ccc(N(c3ccc4c(c3)c3ccccc3n4-c3ccccc3)c3cccc4ccc5nc(-c6ccccc6)oc5c34)cc2)cc1 ; c1ccc(-c2ccc(N(c3ccc4c(c3)c3ccccc3n4-c3ccccc3)c3cccc4ccc5oc(-c6ccccc6)nc5c34)cc2)cc1 ; c1ccc(-c2cccc(N(c3ccc4c(c3)c3ccccc3n4-c3ccccc3)c3cccc4ccc5nc(-c6ccccc6)oc5c34)c2)cc1]

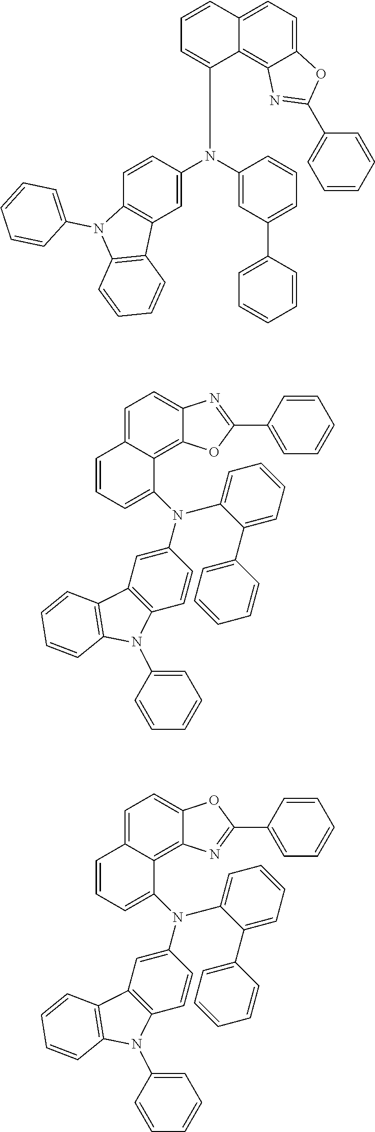 [SMILES: c1ccc(-c2cccc(N(c3ccc4c(c3)c3ccccc3n4-c3ccccc3)c3cccc4ccc5oc(-c6ccccc6)nc5c34)c2)cc1 ; c1ccc(-c2nc3ccc4cccc(N(c5ccc6c(c5)c5ccccc5n6-c5ccccc5)c5ccccc5-c5ccccc5)c4c3o2)cc1 ; c1ccc(-c2nc3c(ccc4cccc(N(c5ccc6c(c5)c5ccccc5n6-c5ccccc5)c5ccccc5-c5ccccc5)c43)o2)cc1]

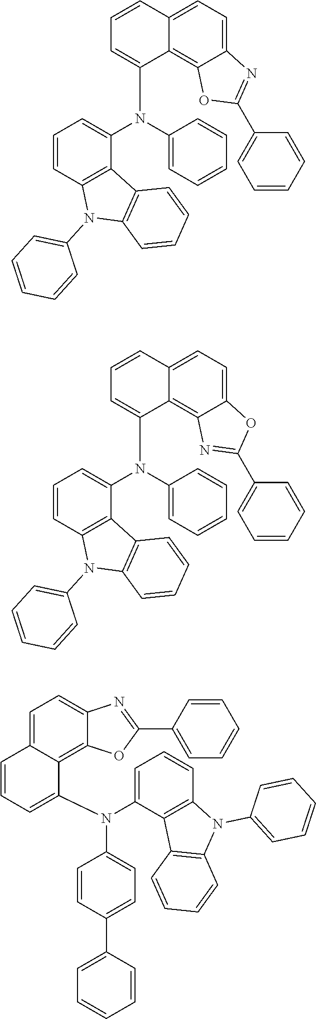

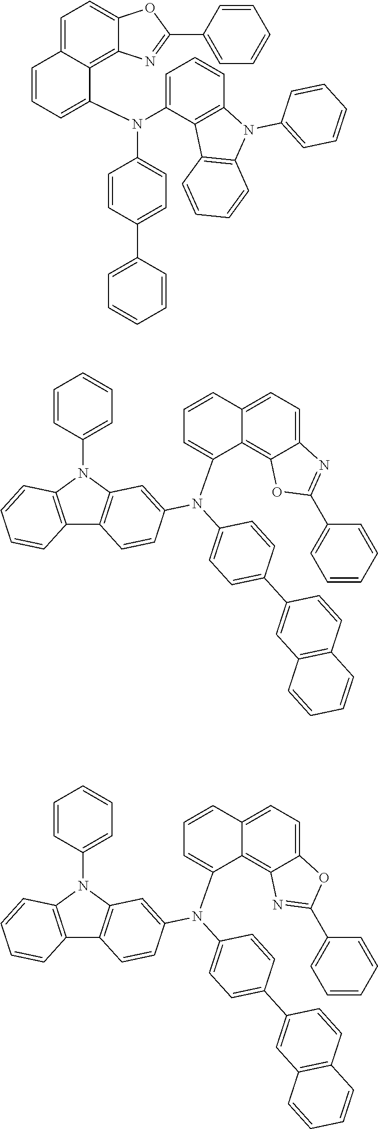 [SMILES: c1ccc(-c2ccc(N(c3cccc4ccc5oc(-c6ccccc6)nc5c34)c3cccc4c3c3ccccc3n4-c3ccccc3)cc2)cc1 ; c1ccc(-c2nc3ccc4cccc(N(c5ccc(-c6ccc7ccccc7c6)cc5)c5ccc6c7ccccc7n(-c7ccccc7)c6c5)c4c3o2)cc1 ; c1ccc(-c2nc3c(ccc4cccc(N(c5ccc(-c6ccc7ccccc7c6)cc5)c5ccc6c7ccccc7n(-c7ccccc7)c6c5)c43)o2)cc1]

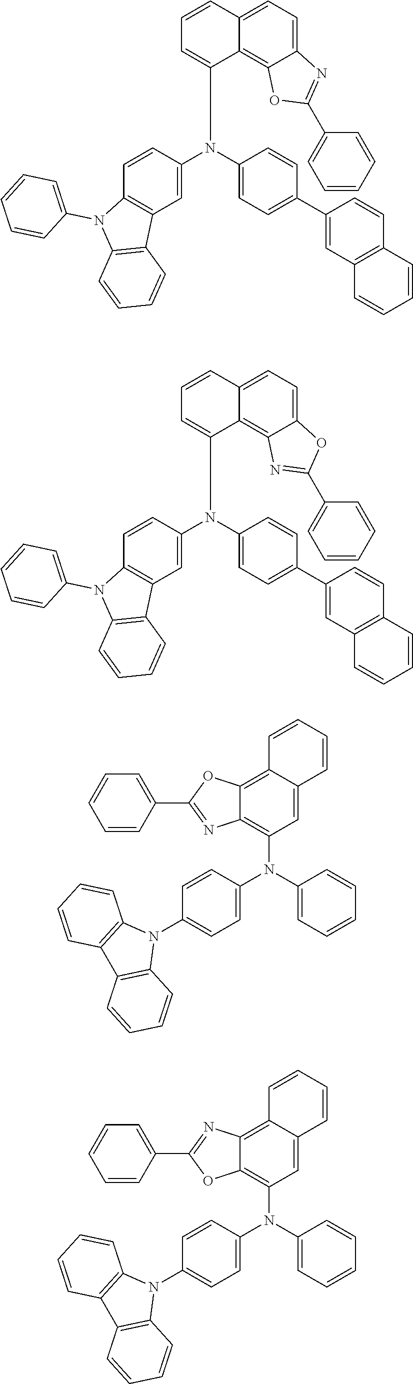 [SMILES: c1ccc(-c2nc3ccc4cccc(N(c5ccc(-c6ccc7ccccc7c6)cc5)c5ccc6c(c5)c5ccccc5n6-c5ccccc5)c4c3o2)cc1 ; c1ccc(-c2nc3c(ccc4cccc(N(c5ccc(-c6ccc7ccccc7c6)cc5)c5ccc6c(c5)c5ccccc5n6-c5ccccc5)c43)o2)cc1 ; c1ccc(-c2nc3c(N(c4ccccc4)c4ccc(-n5c6ccccc6c6ccccc65)cc4)cc4ccccc4c3o2)cc1 ; c1ccc(-c2nc3c(o2)c(N(c2ccccc2)c2ccc(-n4c5ccccc5c5ccccc54)cc2)cc2ccccc23)cc1]

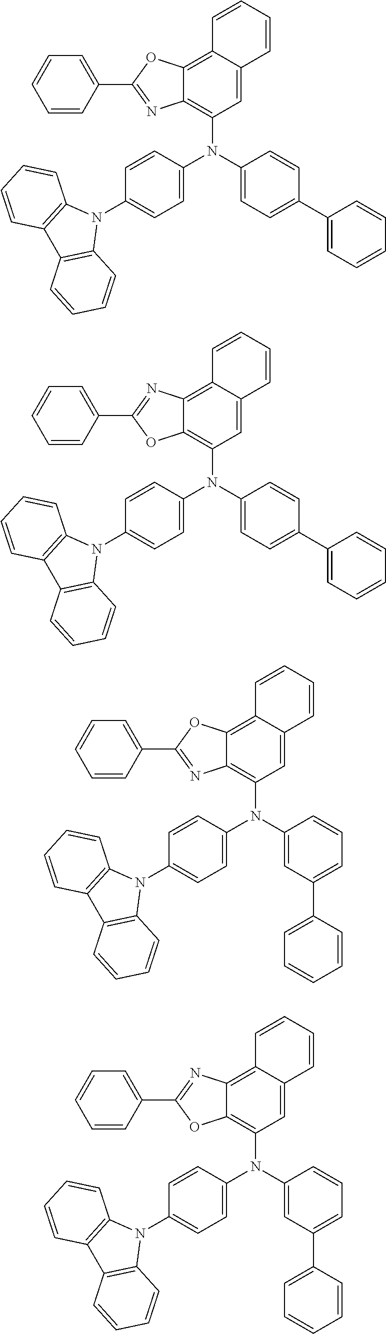

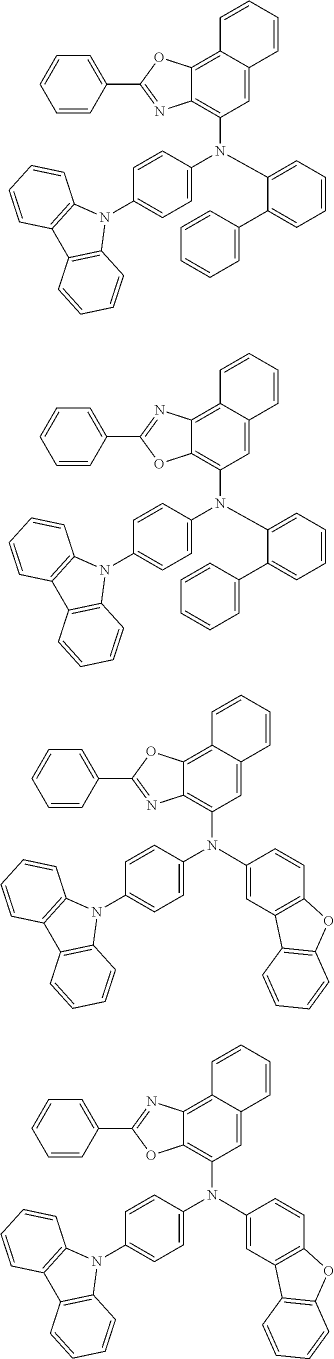 [SMILES: c1ccc(-c2nc3c(N(c4ccc(-n5c6ccccc6c6ccccc65)cc4)c4ccccc4-c4ccccc4)cc4ccccc4c3o2)cc1 ; c1ccc(-c2nc3c(o2)c(N(c2ccc(-n4c5ccccc5c5ccccc54)cc2)c2ccccc2-c2ccccc2)cc2ccccc23)cc1 ; c1ccc(-c2nc3c(N(c4ccc(-n5c6ccccc6c6ccccc65)cc4)c4ccc5oc6ccccc6c5c4)cc4ccccc4c3o2)cc1 ; c1ccc(-c2nc3c(o2)c(N(c2ccc(-n4c5ccccc5c5ccccc54)cc2)c2ccc4oc5ccccc5c4c2)cc2ccccc23)cc1]

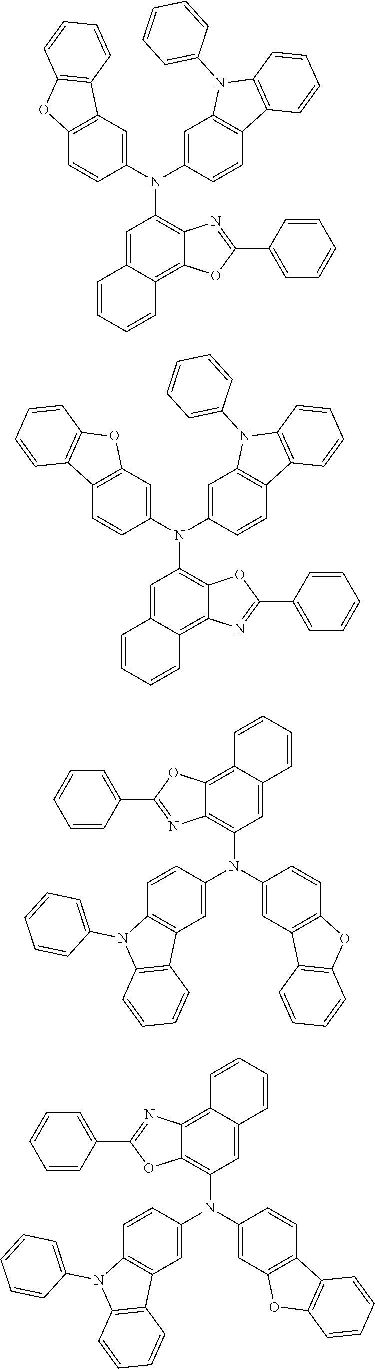 [SMILES: c1ccc(-c2nc3c(N(c4ccc5oc6ccccc6c5c4)c4ccc5c6ccccc6n(-c6ccccc6)c5c4)cc4ccccc4c3o2)cc1 ; c1ccc(-c2nc3c(o2)c(N(c2ccc4c(c2)oc2ccccc24)c2ccc4c5ccccc5n(-c5ccccc5)c4c2)cc2ccccc23)cc1 ; c1ccc(-c2nc3c(N(c4ccc5oc6ccccc6c5c4)c4ccc5c(c4)c4ccccc4n5-c4ccccc4)cc4ccccc4c3o2)cc1 ; c1ccc(-c2nc3c(o2)c(N(c2ccc4c(c2)oc2ccccc24)c2ccc4c(c2)c2ccccc2n4-c2ccccc2)cc2ccccc23)cc1]

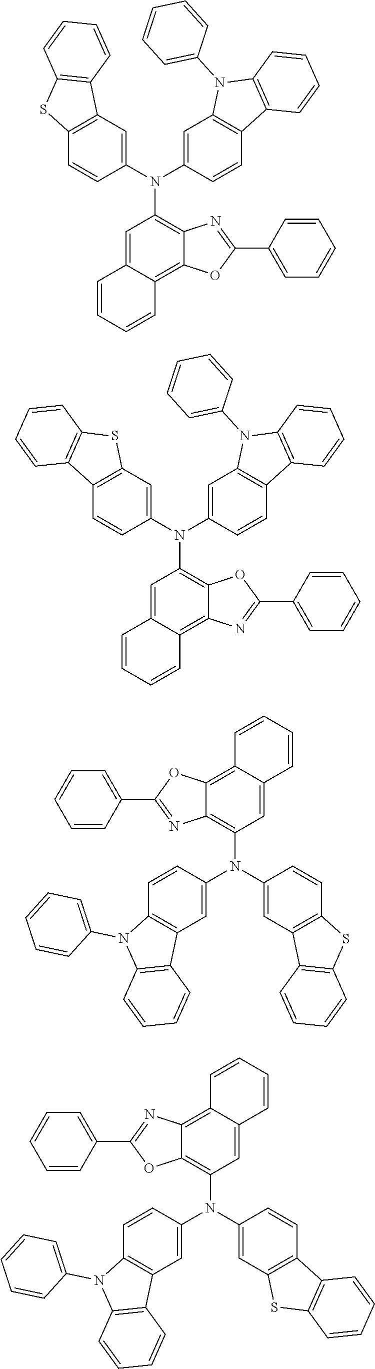 [SMILES: c1ccc(-c2nc3c(N(c4ccc5sc6ccccc6c5c4)c4ccc5c6ccccc6n(-c6ccccc6)c5c4)cc4ccccc4c3o2)cc1 ; c1ccc(-c2nc3c(o2)c(N(c2ccc4c(c2)sc2ccccc24)c2ccc4c5ccccc5n(-c5ccccc5)c4c2)cc2ccccc23)cc1 ; c1ccc(-c2nc3c(N(c4ccc5sc6ccccc6c5c4)c4ccc5c(c4)c4ccccc4n5-c4ccccc4)cc4ccccc4c3o2)cc1 ; c1ccc(-c2nc3c(o2)c(N(c2ccc4c(c2)sc2ccccc24)c2ccc4c(c2)c2ccccc2n4-c2ccccc2)cc2ccccc23)cc1]

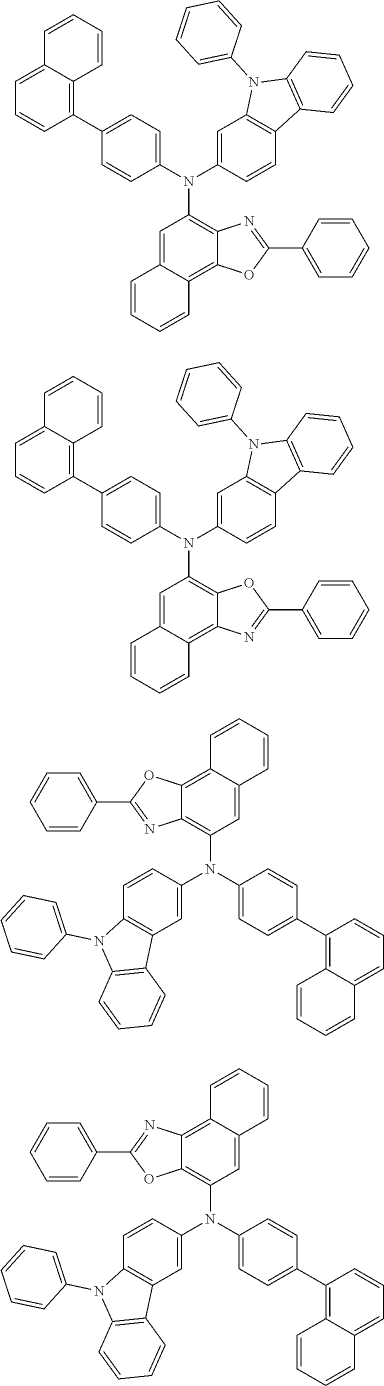

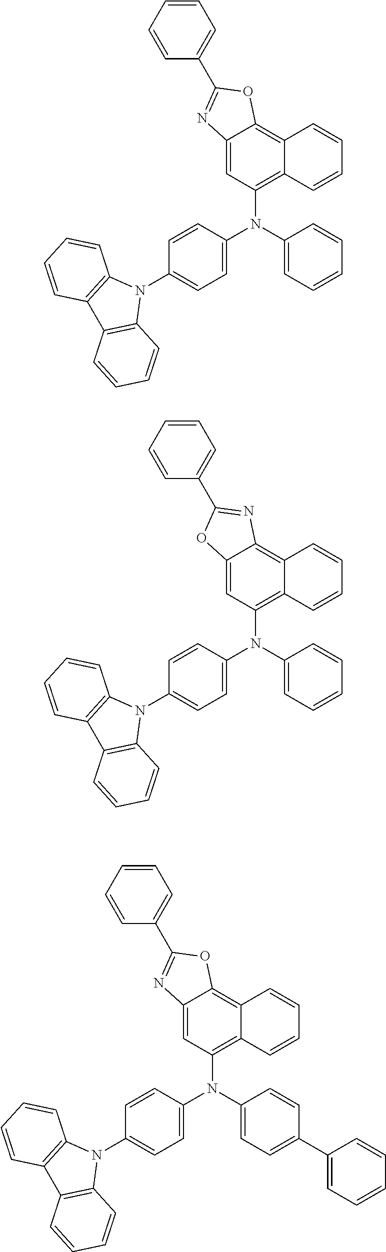

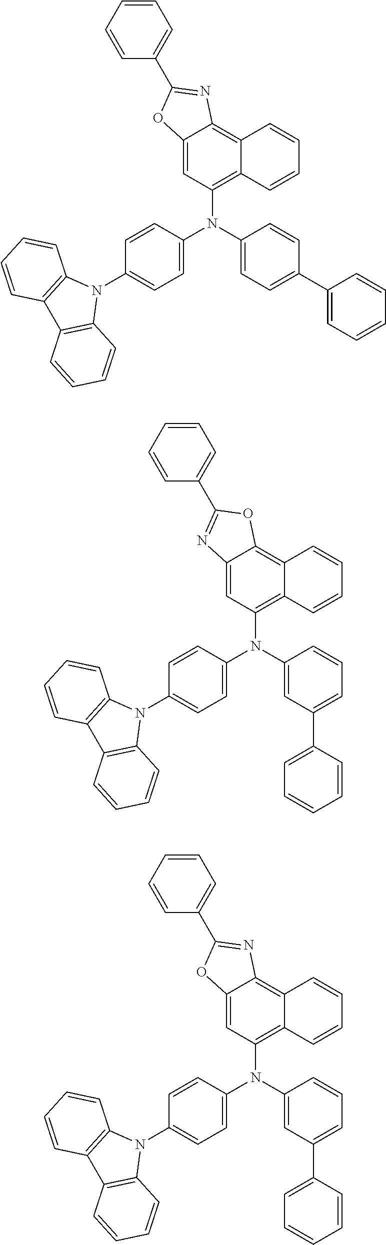 [SMILES: c1ccc(-c2ccc(N(c3ccc(-n4c5ccccc5c5ccccc54)cc3)c3cc4oc(-c5ccccc5)nc4c4ccccc34)cc2)cc1 ; c1ccc(-c2cccc(N(c3ccc(-n4c5ccccc5c5ccccc54)cc3)c3cc4nc(-c5ccccc5)oc4c4ccccc34)c2)cc1 ; c1ccc(-c2cccc(N(c3ccc(-n4c5ccccc5c5ccccc54)cc3)c3cc4oc(-c5ccccc5)nc4c4ccccc34)c2)cc1]

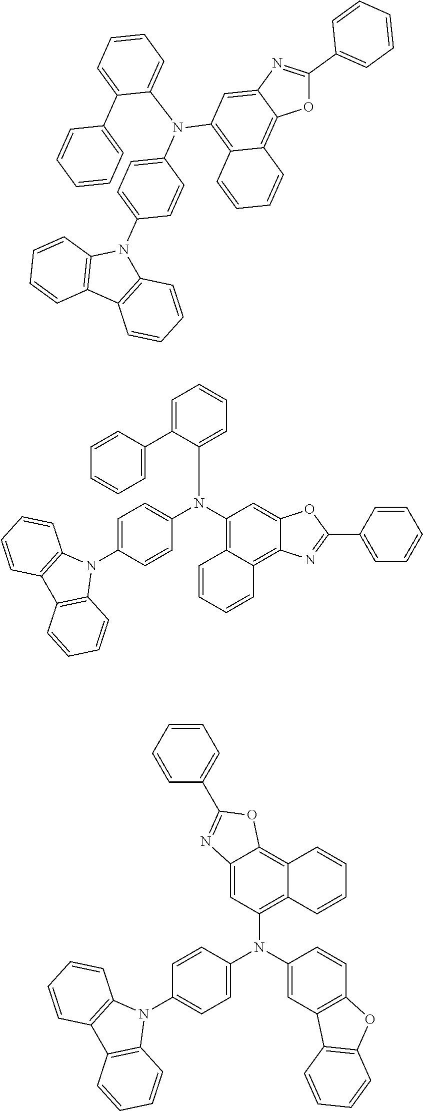 [SMILES: c1ccc(-c2nc3cc(N(c4ccc(-n5c6ccccc6c6ccccc65)cc4)c4ccccc4-c4ccccc4)c4ccccc4c3o2)cc1 ; c1ccc(-c2nc3c(cc(N(c4ccc(-n5c6ccccc6c6ccccc65)cc4)c4ccccc4-c4ccccc4)c4ccccc43)o2)cc1 ; c1ccc(-c2nc3cc(N(c4ccc(-n5c6ccccc6c6ccccc65)cc4)c4ccc5oc6ccccc6c5c4)c4ccccc4c3o2)cc1]

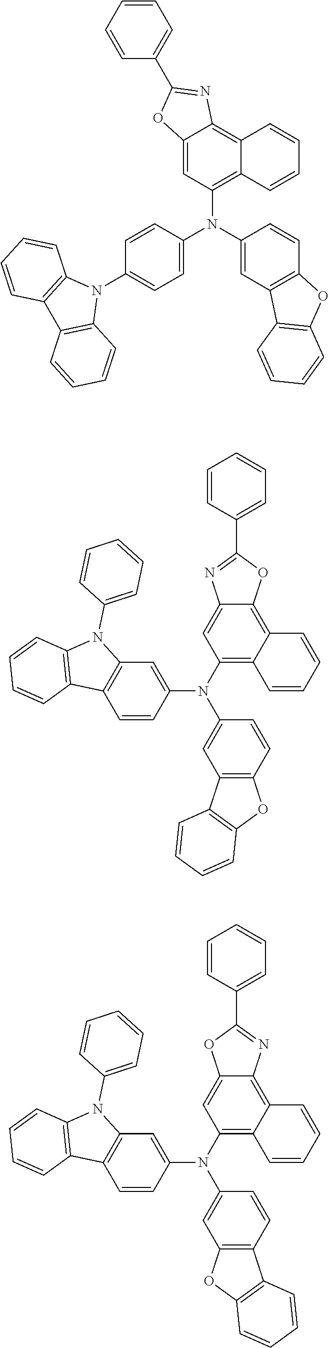 [SMILES: c1ccc(-c2nc3c(cc(N(c4ccc(-n5c6ccccc6c6ccccc65)cc4)c4ccc5oc6ccccc6c5c4)c4ccccc43)o2)cc1 ; c1ccc(-c2nc3cc(N(c4ccc5oc6ccccc6c5c4)c4ccc5c6ccccc6n(-c6ccccc6)c5c4)c4ccccc4c3o2)cc1 ; c1ccc(-c2nc3c(cc(N(c4ccc5c(c4)oc4ccccc45)c4ccc5c6ccccc6n(-c6ccccc6)c5c4)c4ccccc43)o2)cc1]

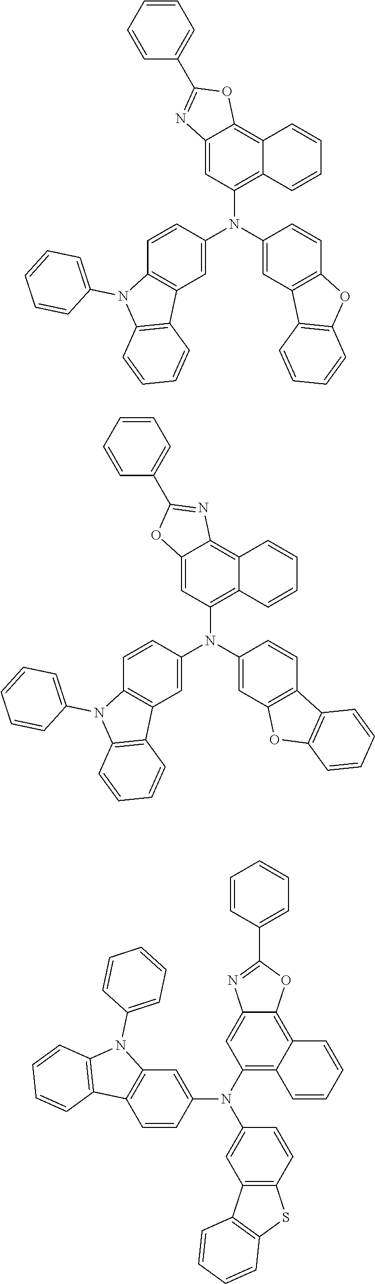

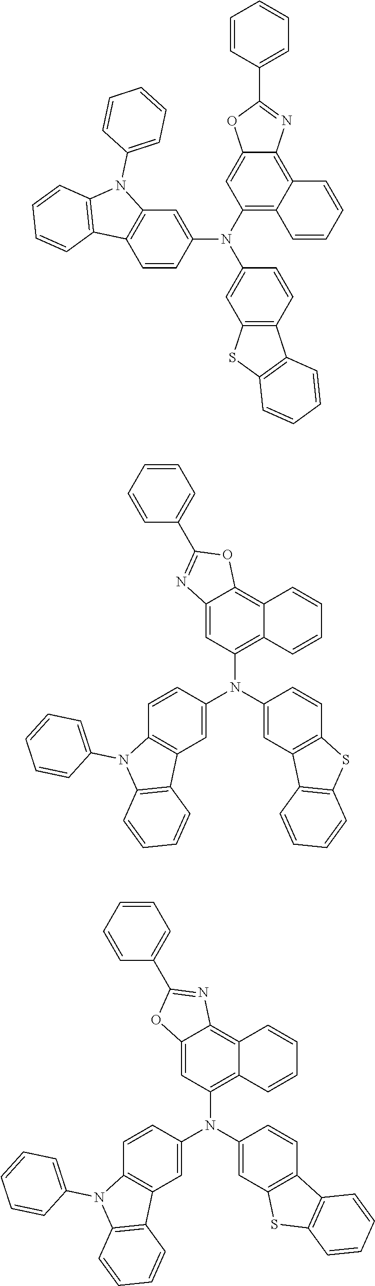 [SMILES: c1ccc(-c2nc3c(cc(N(c4ccc5c(c4)sc4ccccc45)c4ccc5c6ccccc6n(-c6ccccc6)c5c4)c4ccccc43)o2)cc1 ; c1ccc(-c2nc3cc(N(c4ccc5sc6ccccc6c5c4)c4ccc5c(c4)c4ccccc4n5-c4ccccc4)c4ccccc4c3o2)cc1 ; c1ccc(-c2nc3c(cc(N(c4ccc5c(c4)sc4ccccc45)c4ccc5c(c4)c4ccccc4n5-c4ccccc4)c4ccccc43)o2)cc1]

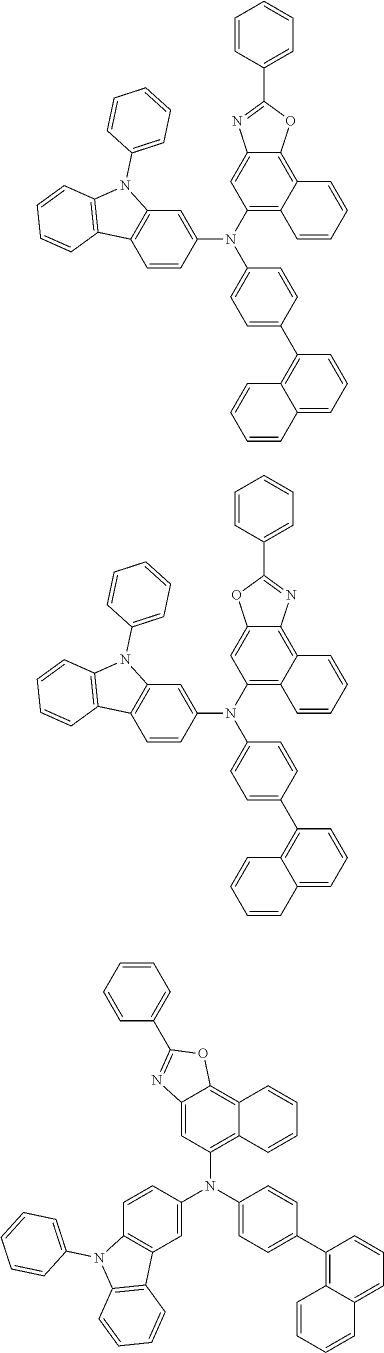

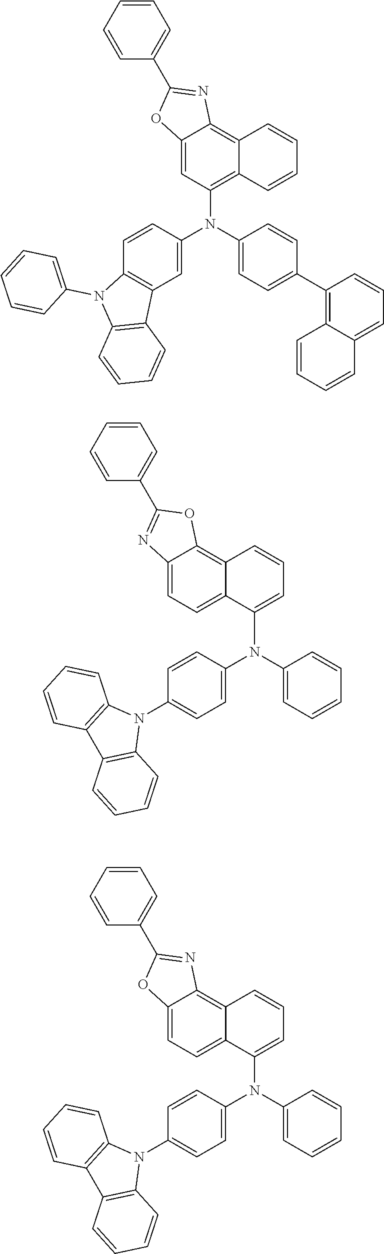

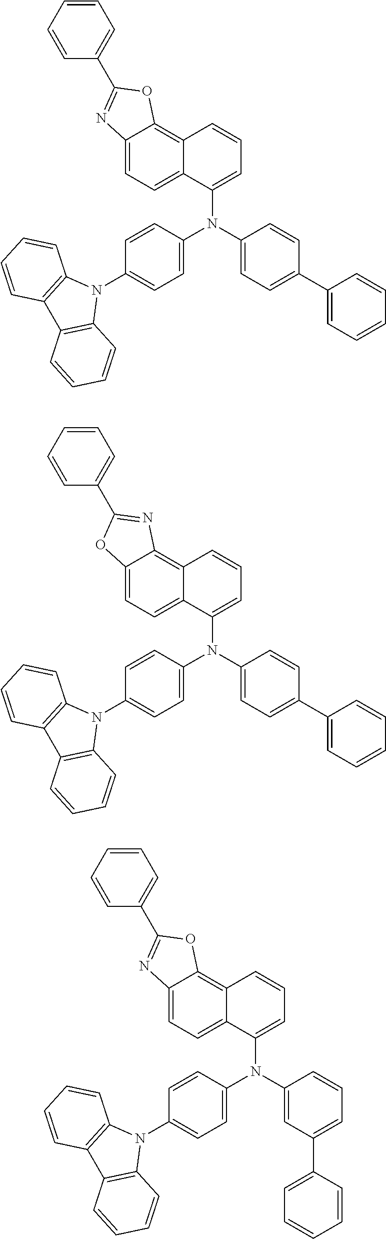

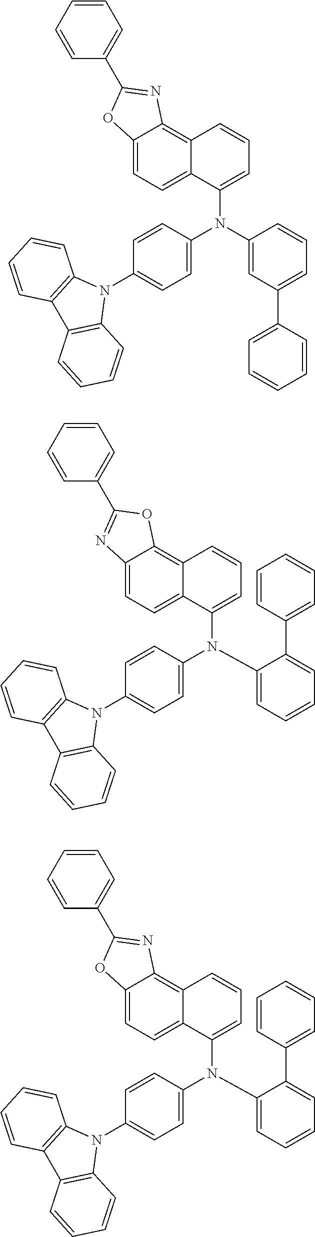

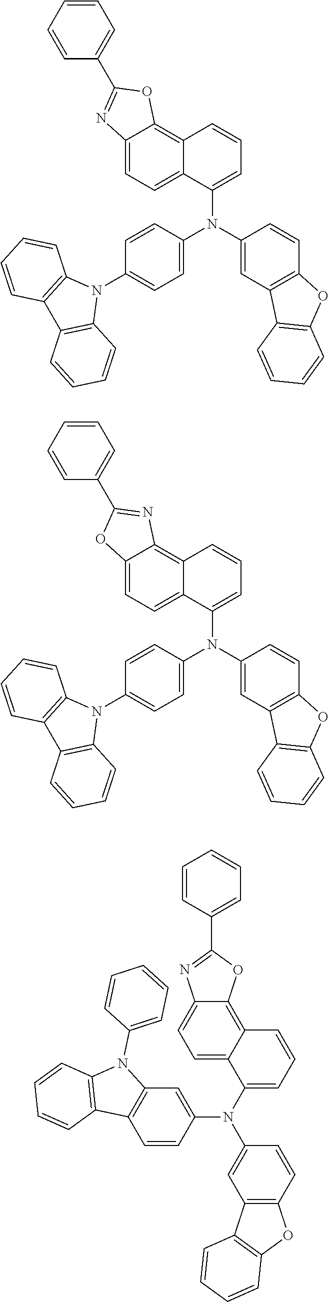

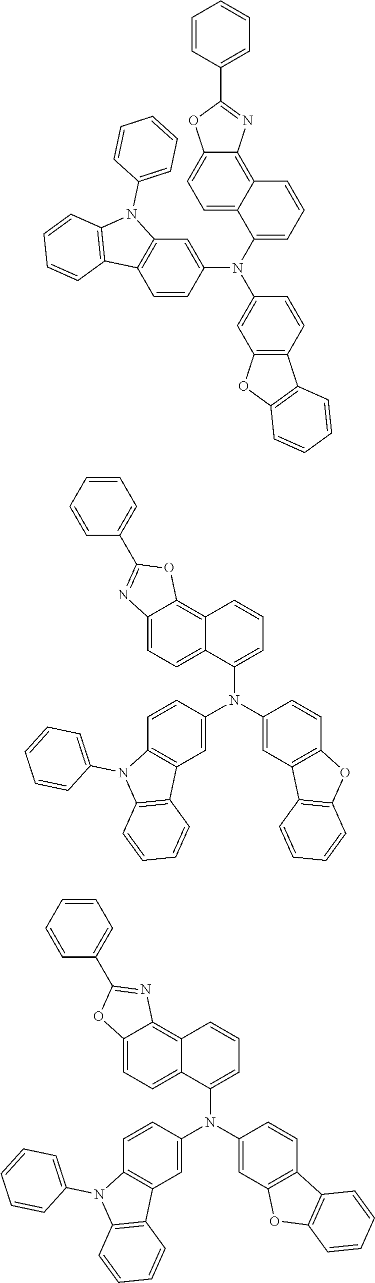 [SMILES: c1ccc(-c2nc3c(ccc4c(N(c5ccc6c(c5)oc5ccccc56)c5ccc6c7ccccc7n(-c7ccccc7)c6c5)cccc43)o2)cc1 ; c1ccc(-c2nc3ccc4c(N(c5ccc6oc7ccccc7c6c5)c5ccc6c(c5)c5ccccc5n6-c5ccccc5)cccc4c3o2)cc1 ; c1ccc(-c2nc3c(ccc4c(N(c5ccc6c(c5)oc5ccccc56)c5ccc6c(c5)c5ccccc5n6-c5ccccc5)cccc43)o2)cc1]

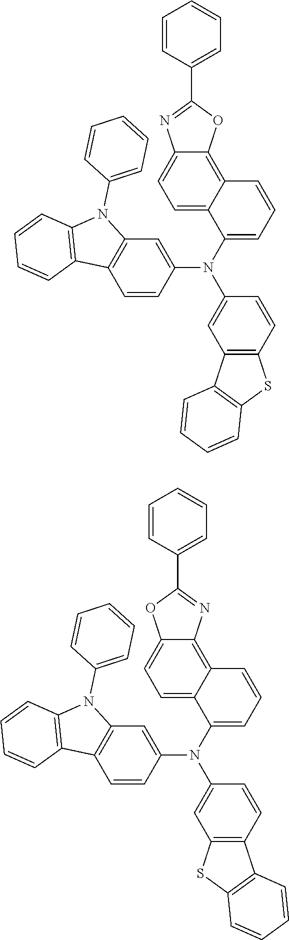 [SMILES: c1ccc(-c2nc3ccc4c(N(c5ccc6sc7ccccc7c6c5)c5ccc6c7ccccc7n(-c7ccccc7)c6c5)cccc4c3o2)cc1 ; c1ccc(-c2nc3c(ccc4c(N(c5ccc6c(c5)sc5ccccc56)c5ccc6c7ccccc7n(-c7ccccc7)c6c5)cccc43)o2)cc1]

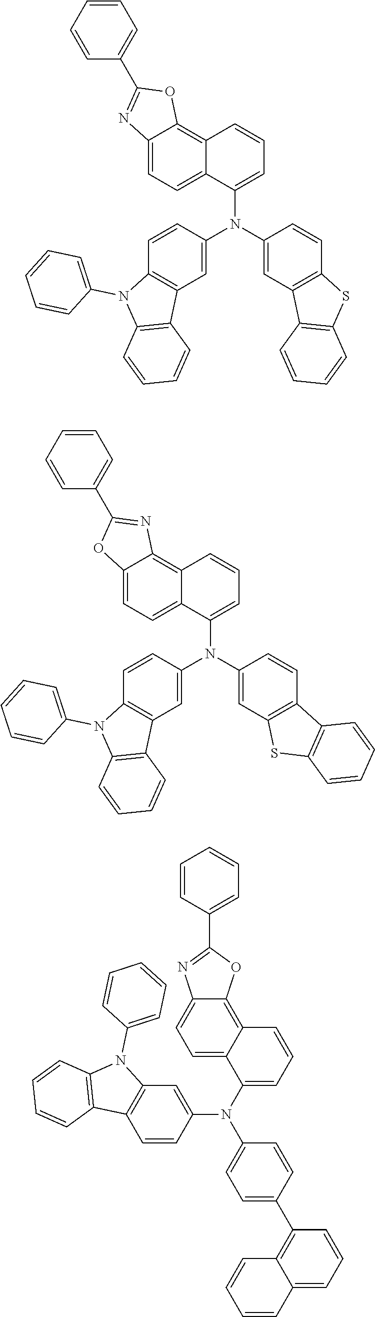 [SMILES: c1ccc(-c2nc3ccc4c(N(c5ccc6sc7ccccc7c6c5)c5ccc6c(c5)c5ccccc5n6-c5ccccc5)cccc4c3o2)cc1 ; c1ccc(-c2nc3c(ccc4c(N(c5ccc6c(c5)sc5ccccc56)c5ccc6c(c5)c5ccccc5n6-c5ccccc5)cccc43)o2)cc1 ; c1ccc(-c2nc3ccc4c(N(c5ccc(-c6cccc7ccccc67)cc5)c5ccc6c7ccccc7n(-c7ccccc7)c6c5)cccc4c3o2)cc1]

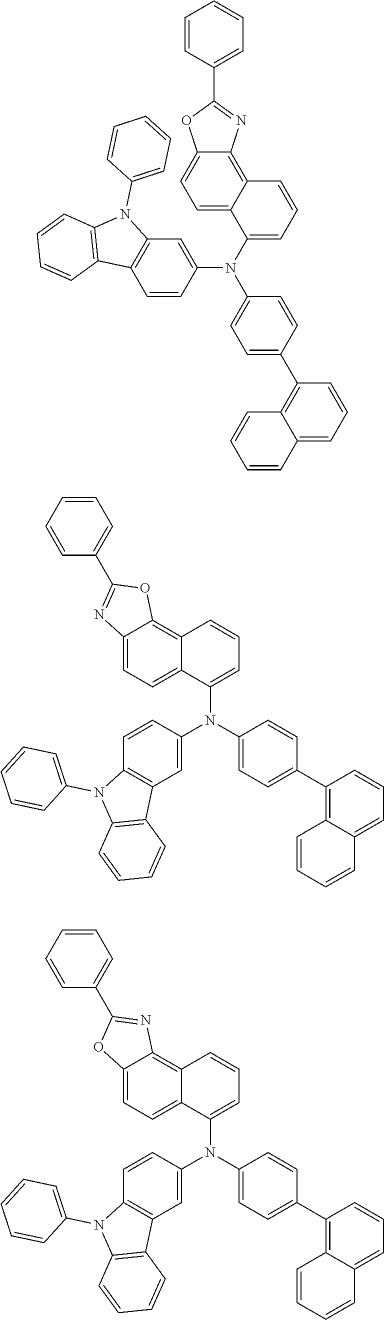 [SMILES: c1ccc(-c2nc3c(ccc4c(N(c5ccc(-c6cccc7ccccc67)cc5)c5ccc6c7ccccc7n(-c7ccccc7)c6c5)cccc43)o2)cc1 ; c1ccc(-c2nc3ccc4c(N(c5ccc(-c6cccc7ccccc67)cc5)c5ccc6c(c5)c5ccccc5n6-c5ccccc5)cccc4c3o2)cc1 ; c1ccc(-c2nc3c(ccc4c(N(c5ccc(-c6cccc7ccccc67)cc5)c5ccc6c(c5)c5ccccc5n6-c5ccccc5)cccc43)o2)cc1]

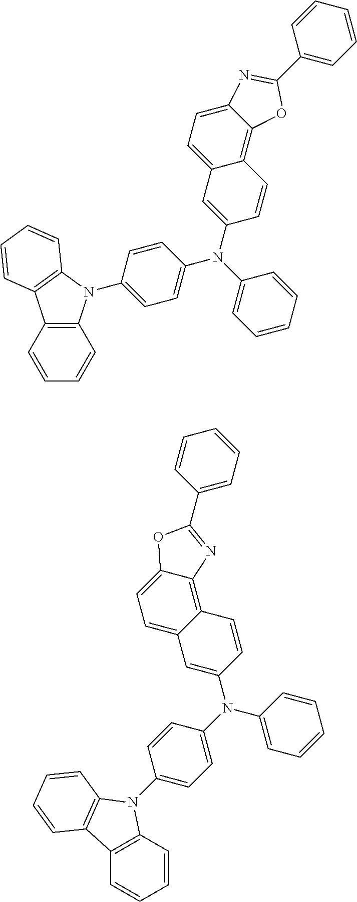 [SMILES: c1ccc(-c2nc3ccc4cc(N(c5ccccc5)c5ccc(-n6c7ccccc7c7ccccc76)cc5)ccc4c3o2)cc1 ; c1ccc(-c2nc3c(ccc4cc(N(c5ccccc5)c5ccc(-n6c7ccccc7c7ccccc76)cc5)ccc43)o2)cc1]

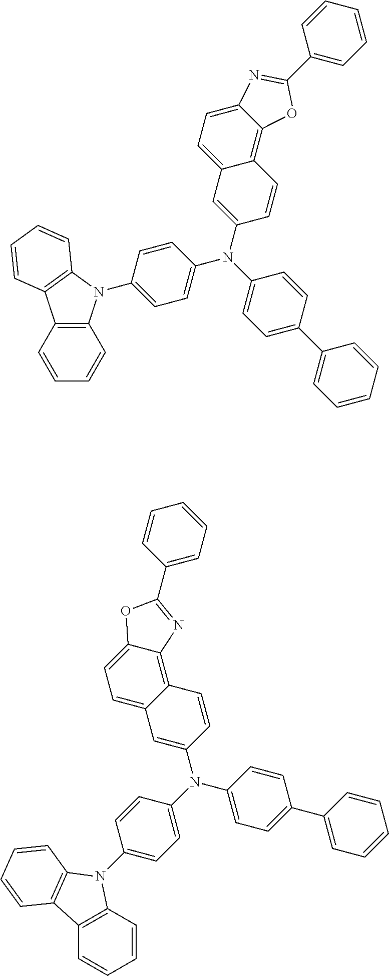 [SMILES: c1ccc(-c2ccc(N(c3ccc(-n4c5ccccc5c5ccccc54)cc3)c3ccc4c(ccc5nc(-c6ccccc6)oc54)c3)cc2)cc1 ; c1ccc(-c2ccc(N(c3ccc(-n4c5ccccc5c5ccccc54)cc3)c3ccc4c(ccc5oc(-c6ccccc6)nc54)c3)cc2)cc1]

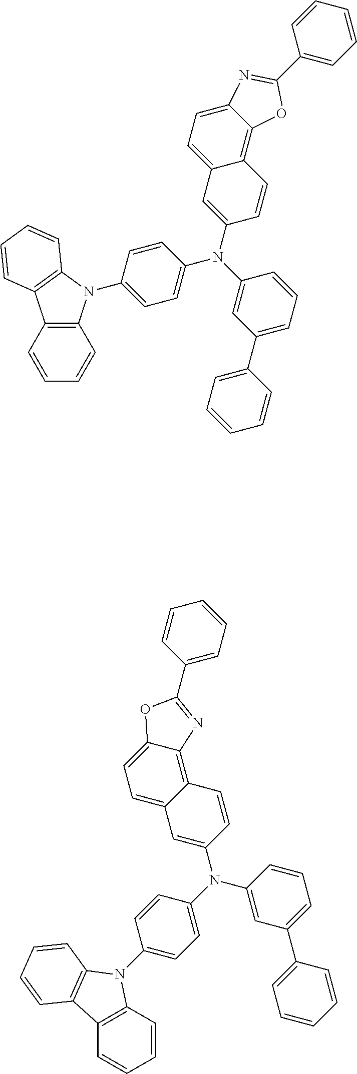 [SMILES: c1ccc(-c2cccc(N(c3ccc(-n4c5ccccc5c5ccccc54)cc3)c3ccc4c(ccc5nc(-c6ccccc6)oc54)c3)c2)cc1 ; c1ccc(-c2cccc(N(c3ccc(-n4c5ccccc5c5ccccc54)cc3)c3ccc4c(ccc5oc(-c6ccccc6)nc54)c3)c2)cc1]

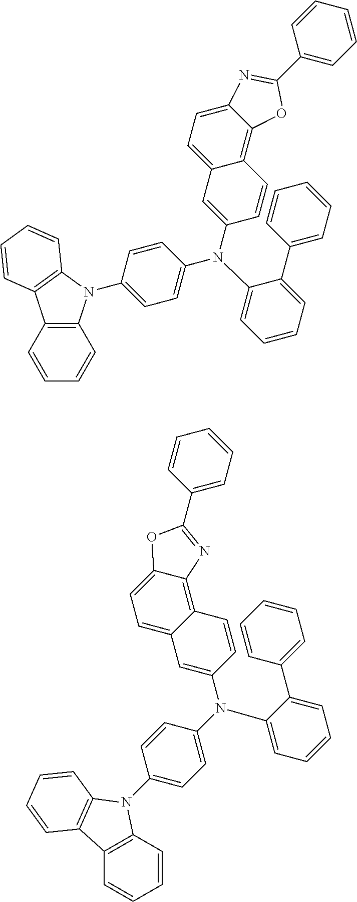 [SMILES: c1ccc(-c2nc3ccc4cc(N(c5ccc(-n6c7ccccc7c7ccccc76)cc5)c5ccccc5-c5ccccc5)ccc4c3o2)cc1 ; c1ccc(-c2nc3c(ccc4cc(N(c5ccc(-n6c7ccccc7c7ccccc76)cc5)c5ccccc5-c5ccccc5)ccc43)o2)cc1]

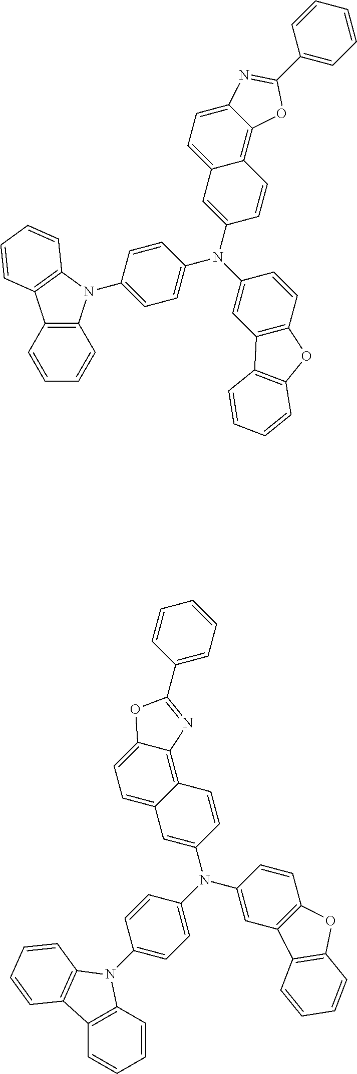 [SMILES: c1ccc(-c2nc3ccc4cc(N(c5ccc(-n6c7ccccc7c7ccccc76)cc5)c5ccc6oc7ccccc7c6c5)ccc4c3o2)cc1 ; c1ccc(-c2nc3c(ccc4cc(N(c5ccc(-n6c7ccccc7c7ccccc76)cc5)c5ccc6oc7ccccc7c6c5)ccc43)o2)cc1]

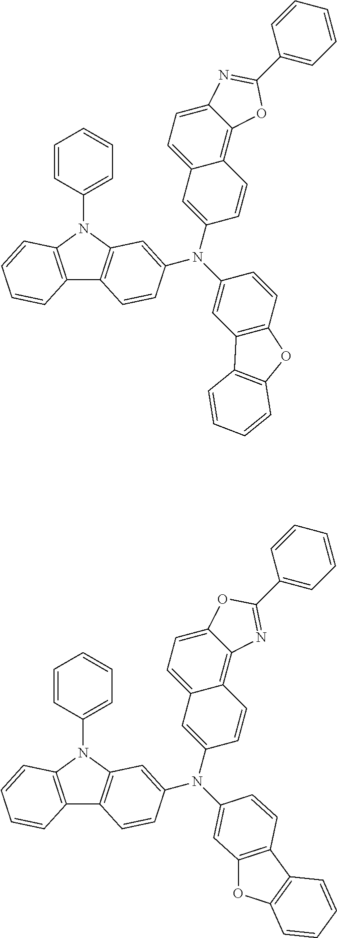 [SMILES: c1ccc(-c2nc3ccc4cc(N(c5ccc6oc7ccccc7c6c5)c5ccc6c7ccccc7n(-c7ccccc7)c6c5)ccc4c3o2)cc1 ; c1ccc(-c2nc3c(ccc4cc(N(c5ccc6c(c5)oc5ccccc56)c5ccc6c7ccccc7n(-c7ccccc7)c6c5)ccc43)o2)cc1]

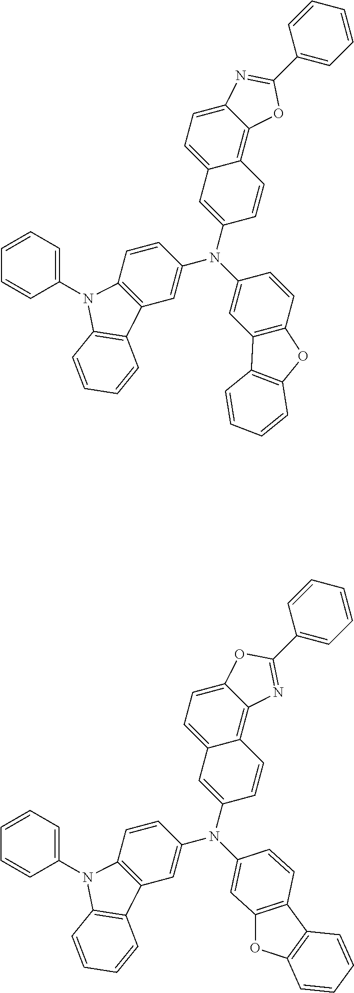 [SMILES: c1ccc(-c2nc3ccc4cc(N(c5ccc6oc7ccccc7c6c5)c5ccc6c(c5)c5ccccc5n6-c5ccccc5)ccc4c3o2)cc1 ; c1ccc(-c2nc3c(ccc4cc(N(c5ccc6c(c5)oc5ccccc56)c5ccc6c(c5)c5ccccc5n6-c5ccccc5)ccc43)o2)cc1]

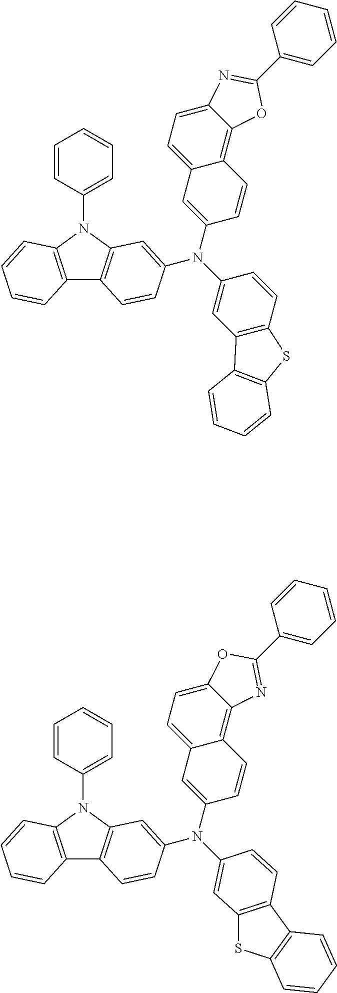 [SMILES: c1ccc(-c2nc3ccc4cc(N(c5ccc6sc7ccccc7c6c5)c5ccc6c7ccccc7n(-c7ccccc7)c6c5)ccc4c3o2)cc1 ; c1ccc(-c2nc3c(ccc4cc(N(c5ccc6c(c5)sc5ccccc56)c5ccc6c7ccccc7n(-c7ccccc7)c6c5)ccc43)o2)cc1]

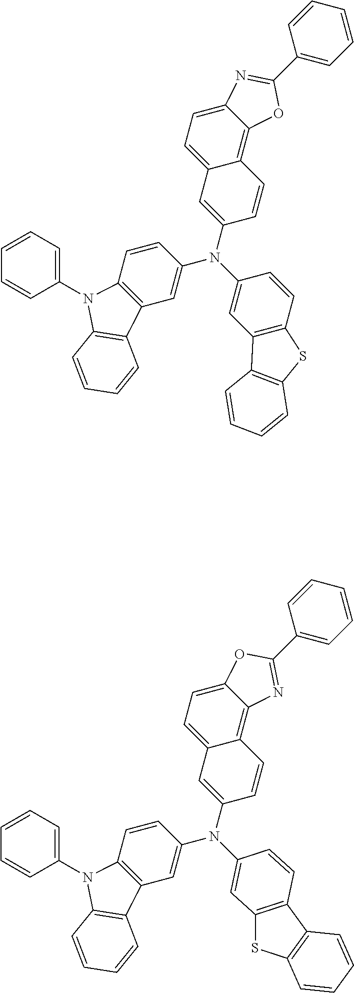 [SMILES: c1ccc(-c2nc3ccc4cc(N(c5ccc6sc7ccccc7c6c5)c5ccc6c(c5)c5ccccc5n6-c5ccccc5)ccc4c3o2)cc1 ; c1ccc(-c2nc3c(ccc4cc(N(c5ccc6c(c5)sc5ccccc56)c5ccc6c(c5)c5ccccc5n6-c5ccccc5)ccc43)o2)cc1]

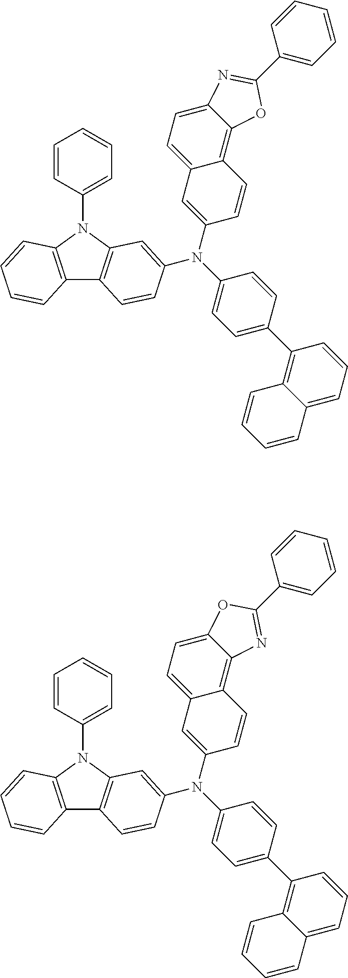 [SMILES: c1ccc(-c2nc3ccc4cc(N(c5ccc(-c6cccc7ccccc67)cc5)c5ccc6c7ccccc7n(-c7ccccc7)c6c5)ccc4c3o2)cc1 ; c1ccc(-c2nc3c(ccc4cc(N(c5ccc(-c6cccc7ccccc67)cc5)c5ccc6c7ccccc7n(-c7ccccc7)c6c5)ccc43)o2)cc1]

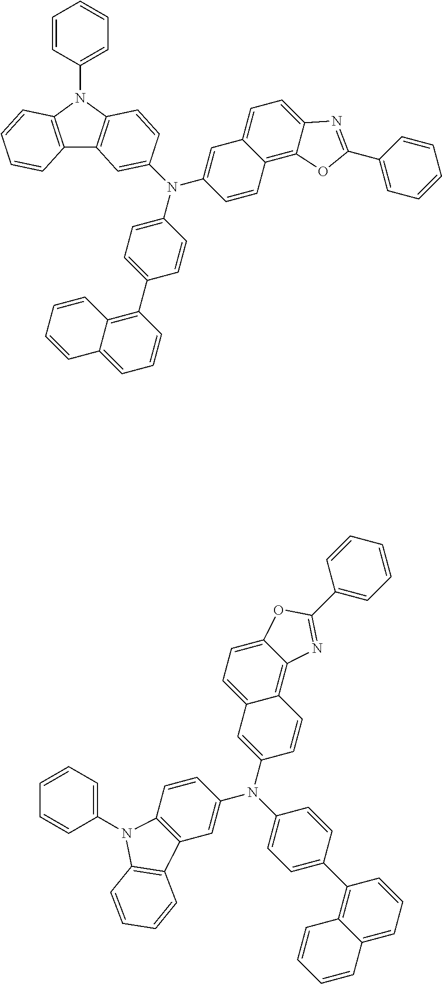 [SMILES: c1ccc(-c2nc3ccc4cc(N(c5ccc(-c6cccc7ccccc67)cc5)c5ccc6c(c5)c5ccccc5n6-c5ccccc5)ccc4c3o2)cc1 ; c1ccc(-c2nc3c(ccc4cc(N(c5ccc(-c6cccc7ccccc67)cc5)c5ccc6c(c5)c5ccccc5n6-c5ccccc5)ccc43)o2)cc1]

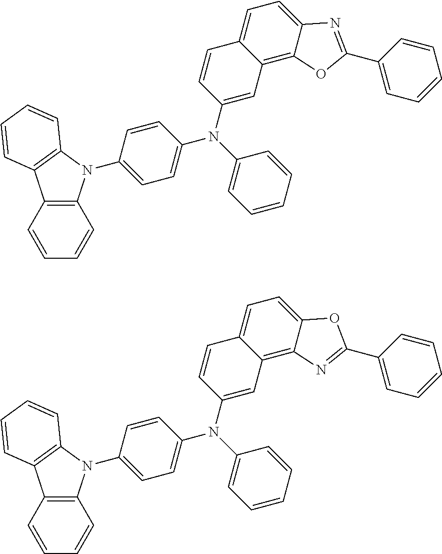 [SMILES: c1ccc(-c2nc3ccc4ccc(N(c5ccccc5)c5ccc(-n6c7ccccc7c7ccccc76)cc5)cc4c3o2)cc1 ; c1ccc(-c2nc3c(ccc4ccc(N(c5ccccc5)c5ccc(-n6c7ccccc7c7ccccc76)cc5)cc43)o2)cc1]

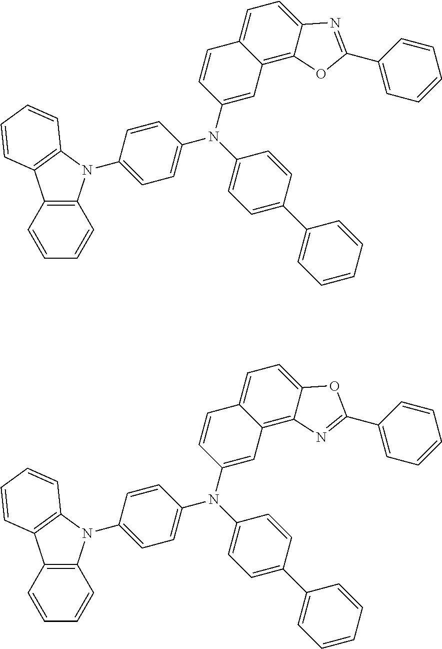 [SMILES: c1ccc(-c2ccc(N(c3ccc(-n4c5ccccc5c5ccccc54)cc3)c3ccc4ccc5nc(-c6ccccc6)oc5c4c3)cc2)cc1 ; c1ccc(-c2ccc(N(c3ccc(-n4c5ccccc5c5ccccc54)cc3)c3ccc4ccc5oc(-c6ccccc6)nc5c4c3)cc2)cc1]

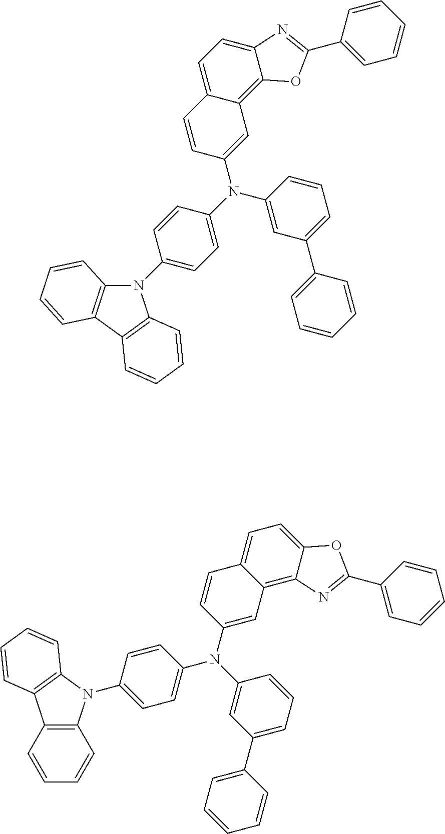 [SMILES: c1ccc(-c2cccc(N(c3ccc(-n4c5ccccc5c5ccccc54)cc3)c3ccc4ccc5nc(-c6ccccc6)oc5c4c3)c2)cc1 ; c1ccc(-c2cccc(N(c3ccc(-n4c5ccccc5c5ccccc54)cc3)c3ccc4ccc5oc(-c6ccccc6)nc5c4c3)c2)cc1]

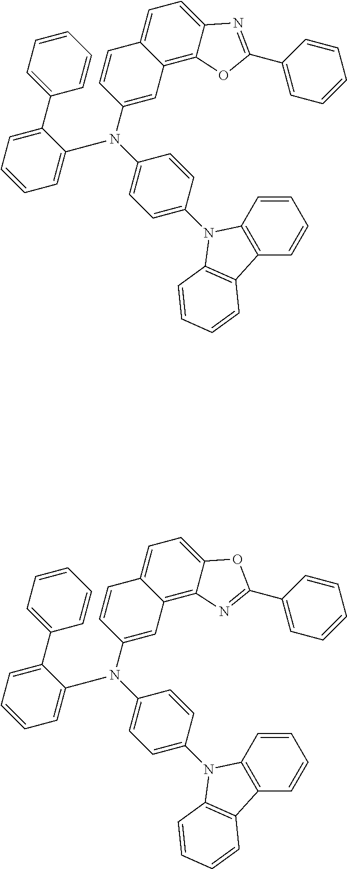 [SMILES: c1ccc(-c2nc3ccc4ccc(N(c5ccc(-n6c7ccccc7c7ccccc76)cc5)c5ccccc5-c5ccccc5)cc4c3o2)cc1 ; c1ccc(-c2nc3c(ccc4ccc(N(c5ccc(-n6c7ccccc7c7ccccc76)cc5)c5ccccc5-c5ccccc5)cc43)o2)cc1]

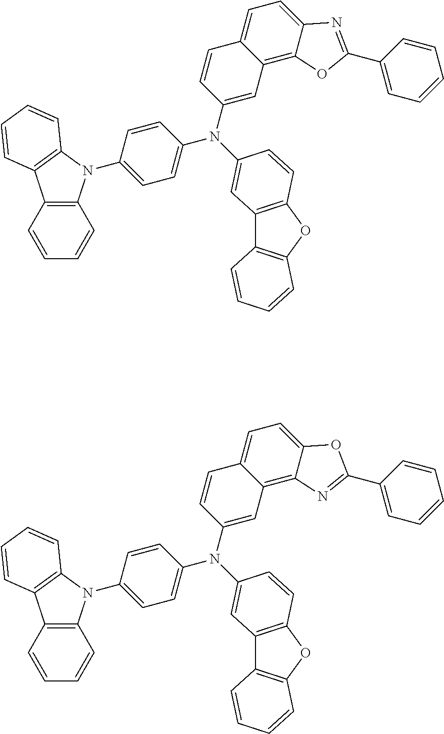 [SMILES: c1ccc(-c2nc3ccc4ccc(N(c5ccc(-n6c7ccccc7c7ccccc76)cc5)c5ccc6oc7ccccc7c6c5)cc4c3o2)cc1 ; c1ccc(-c2nc3c(ccc4ccc(N(c5ccc(-n6c7ccccc7c7ccccc76)cc5)c5ccc6oc7ccccc7c6c5)cc43)o2)cc1]

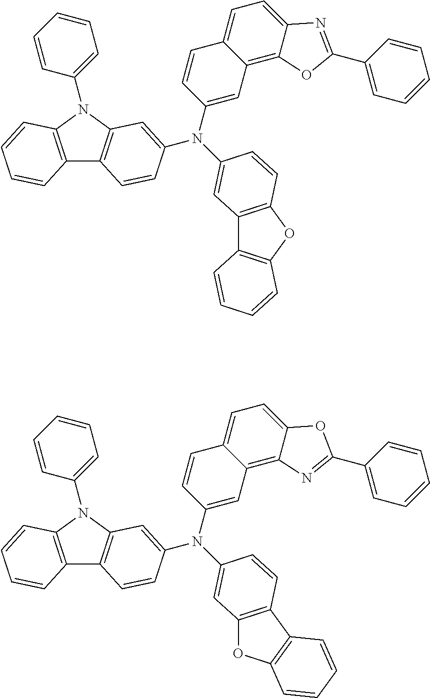 [SMILES: c1ccc(-c2nc3ccc4ccc(N(c5ccc6oc7ccccc7c6c5)c5ccc6c7ccccc7n(-c7ccccc7)c6c5)cc4c3o2)cc1 ; c1ccc(-c2nc3c(ccc4ccc(N(c5ccc6c(c5)oc5ccccc56)c5ccc6c7ccccc7n(-c7ccccc7)c6c5)cc43)o2)cc1]

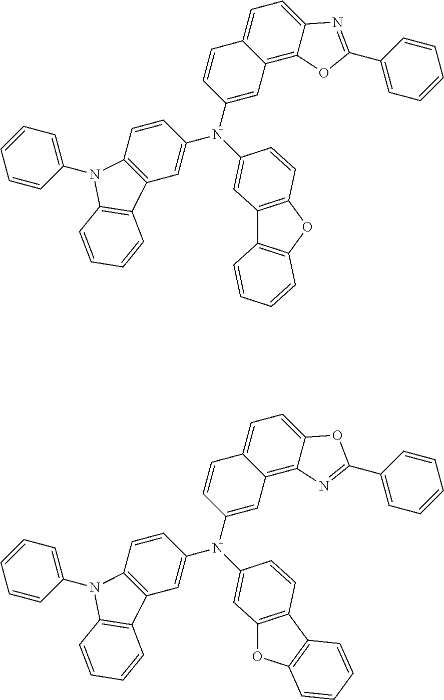 [SMILES: c1ccc(-c2nc3ccc4ccc(N(c5ccc6oc7ccccc7c6c5)c5ccc6c(c5)c5ccccc5n6-c5ccccc5)cc4c3o2)cc1 ; c1ccc(-c2nc3c(ccc4ccc(N(c5ccc6c(c5)oc5ccccc56)c5ccc6c(c5)c5ccccc5n6-c5ccccc5)cc43)o2)cc1]

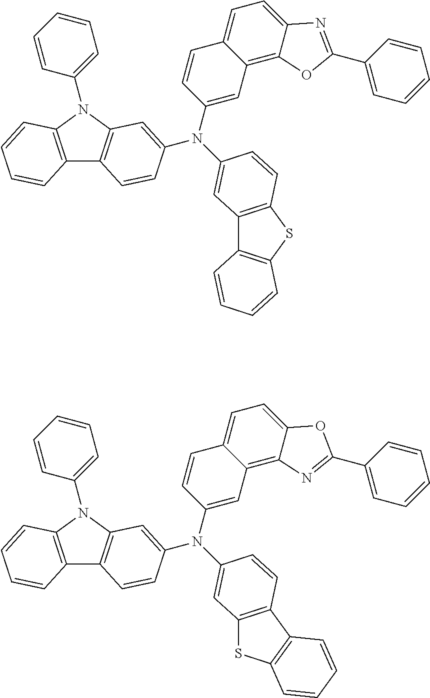 [SMILES: c1ccc(-c2nc3ccc4ccc(N(c5ccc6sc7ccccc7c6c5)c5ccc6c7ccccc7n(-c7ccccc7)c6c5)cc4c3o2)cc1 ; c1ccc(-c2nc3c(ccc4ccc(N(c5ccc6c(c5)sc5ccccc56)c5ccc6c7ccccc7n(-c7ccccc7)c6c5)cc43)o2)cc1]

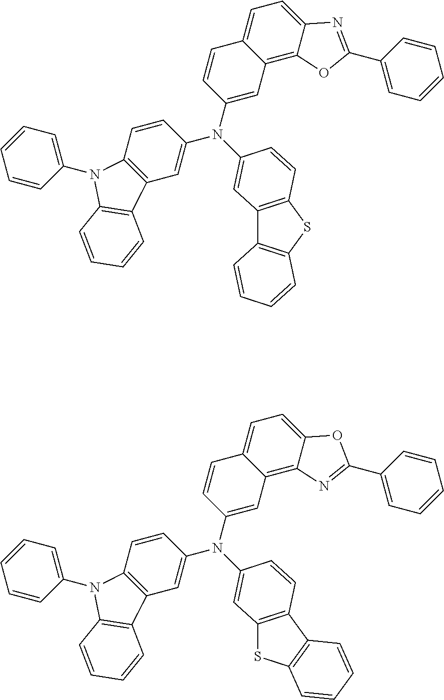 [SMILES: c1ccc(-c2nc3ccc4ccc(N(c5ccc6sc7ccccc7c6c5)c5ccc6c(c5)c5ccccc5n6-c5ccccc5)cc4c3o2)cc1 ; c1ccc(-c2nc3c(ccc4ccc(N(c5ccc6c(c5)sc5ccccc56)c5ccc6c(c5)c5ccccc5n6-c5ccccc5)cc43)o2)cc1]

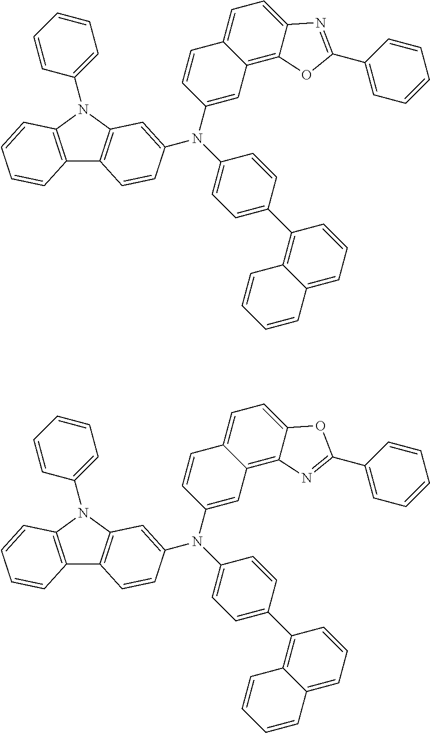 [SMILES: c1ccc(-c2nc3ccc4ccc(N(c5ccc(-c6cccc7ccccc67)cc5)c5ccc6c7ccccc7n(-c7ccccc7)c6c5)cc4c3o2)cc1 ; c1ccc(-c2nc3c(ccc4ccc(N(c5ccc(-c6cccc7ccccc67)cc5)c5ccc6c7ccccc7n(-c7ccccc7)c6c5)cc43)o2)cc1]

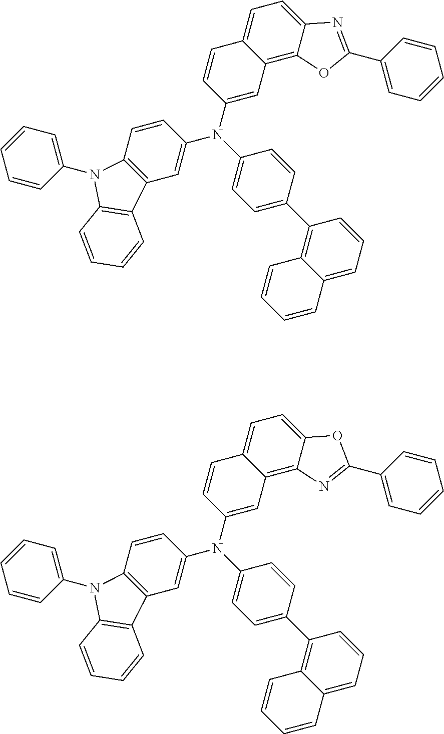 [SMILES: c1ccc(-c2nc3ccc4ccc(N(c5ccc(-c6cccc7ccccc67)cc5)c5ccc6c(c5)c5ccccc5n6-c5ccccc5)cc4c3o2)cc1 ; c1ccc(-c2nc3c(ccc4ccc(N(c5ccc(-c6cccc7ccccc67)cc5)c5ccc6c(c5)c5ccccc5n6-c5ccccc5)cc43)o2)cc1]

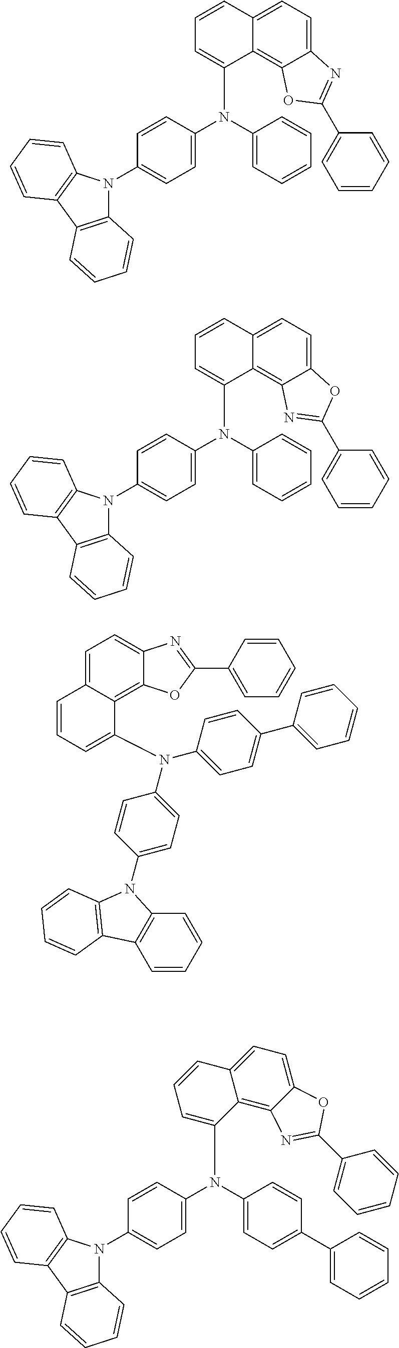

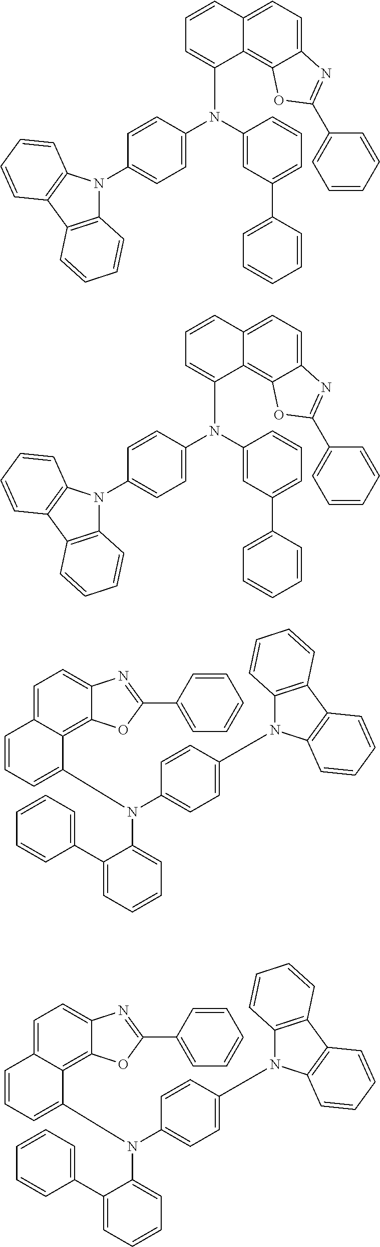

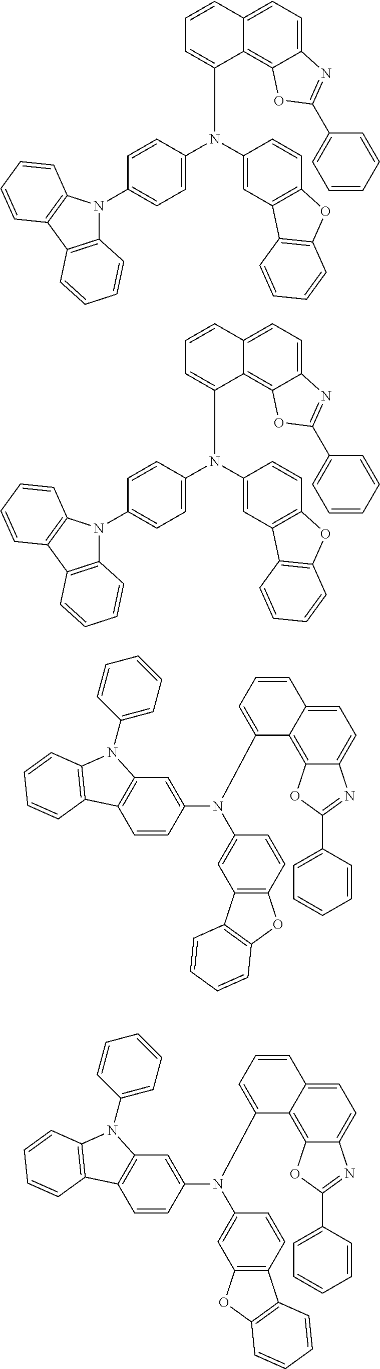

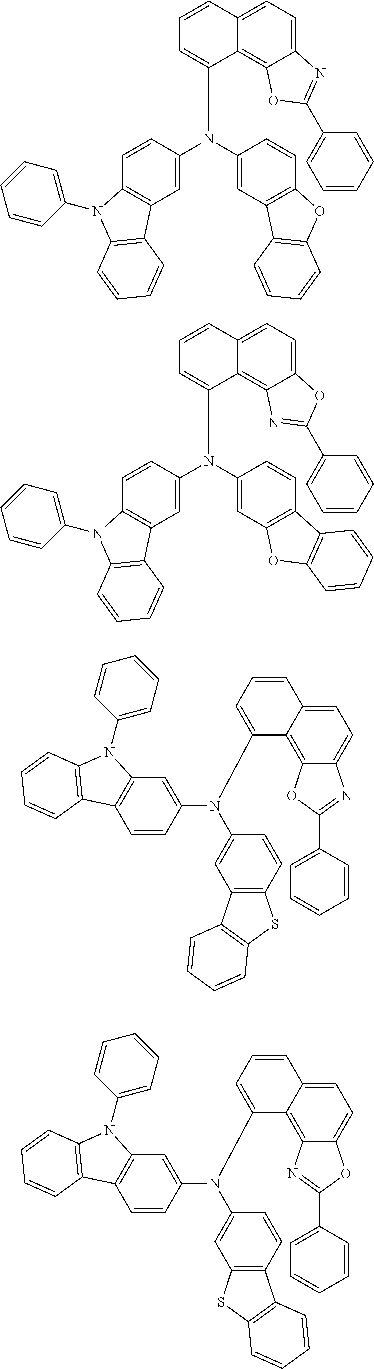

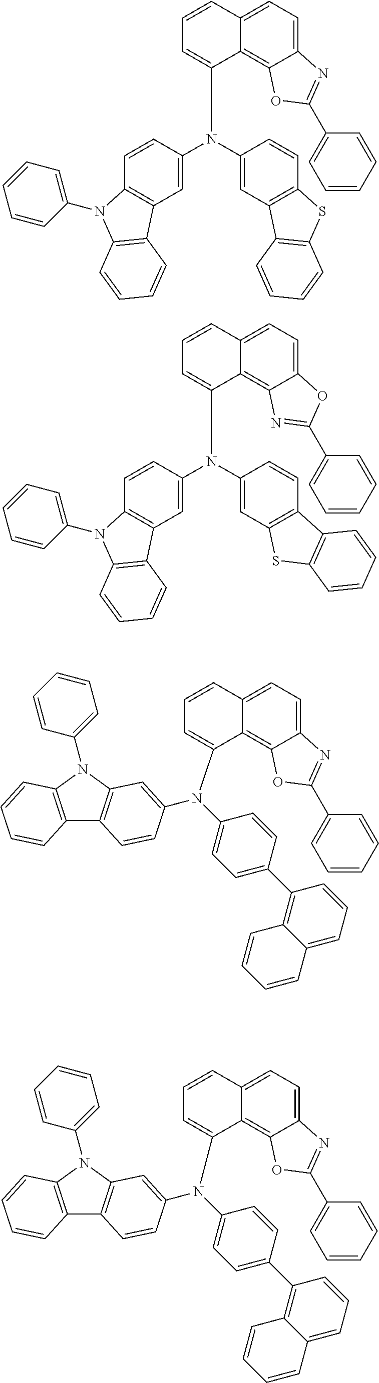

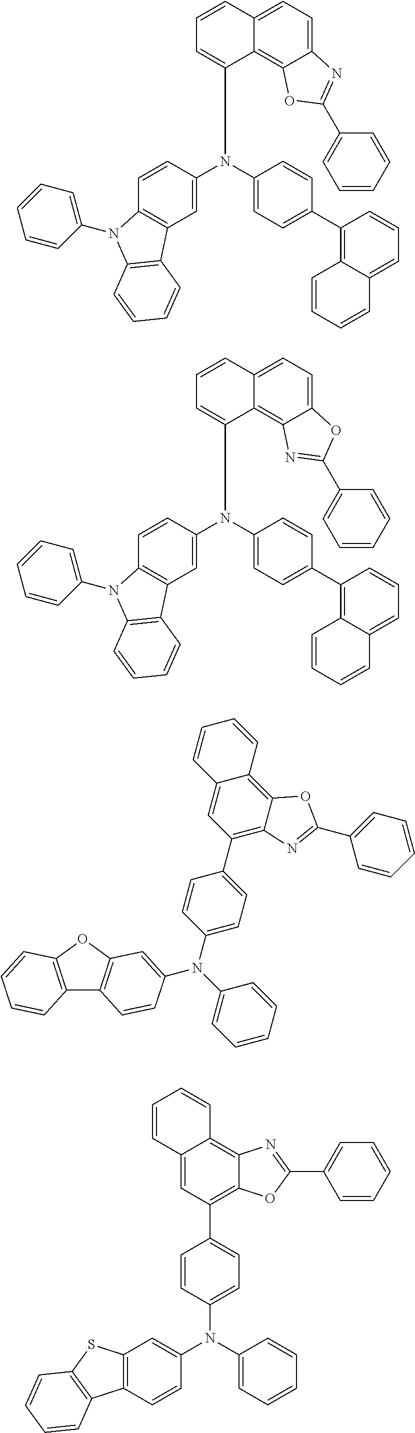 [SMILES: c1ccc(-c2nc3ccc4cccc(N(c5ccc(-c6cccc7ccccc67)cc5)c5ccc6c(c5)c5ccccc5n6-c5ccccc5)c4c3o2)cc1 ; c1ccc(-c2nc3c(ccc4cccc(N(c5ccc(-c6cccc7ccccc67)cc5)c5ccc6c(c5)c5ccccc5n6-c5ccccc5)c43)o2)cc1 ; c1ccc(-c2nc3c(-c4ccc(N(c5ccccc5)c5ccc6c(c5)oc5ccccc56)cc4)cc4ccccc4c3o2)cc1 ; c1ccc(-c2nc3c(o2)c(-c2ccc(N(c4ccccc4)c4ccc5c(c4)sc4ccccc45)cc2)cc2ccccc23)cc1]

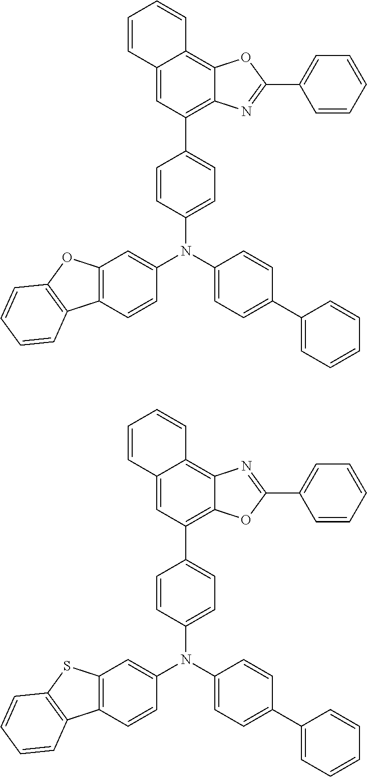 [SMILES: c1ccc(-c2ccc(N(c3ccc(-c4cc5ccccc5c5oc(-c6ccccc6)nc45)cc3)c3ccc4c(c3)oc3ccccc34)cc2)cc1 ; c1ccc(-c2ccc(N(c3ccc(-c4cc5ccccc5c5nc(-c6ccccc6)oc45)cc3)c3ccc4c(c3)sc3ccccc34)cc2)cc1]

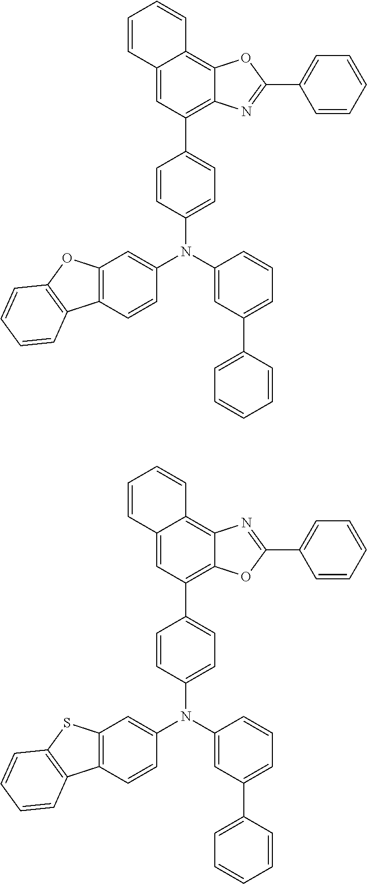 [SMILES: c1ccc(-c2cccc(N(c3ccc(-c4cc5ccccc5c5oc(-c6ccccc6)nc45)cc3)c3ccc4c(c3)oc3ccccc34)c2)cc1 ; c1ccc(-c2cccc(N(c3ccc(-c4cc5ccccc5c5nc(-c6ccccc6)oc45)cc3)c3ccc4c(c3)sc3ccccc34)c2)cc1]

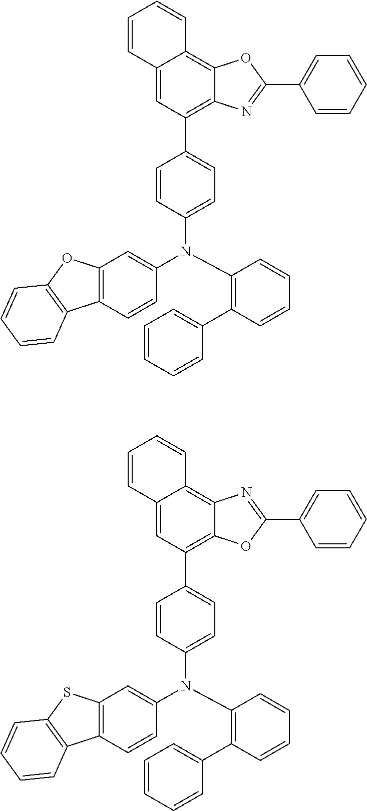 [SMILES: c1ccc(-c2nc3c(-c4ccc(N(c5ccc6c(c5)oc5ccccc56)c5ccccc5-c5ccccc5)cc4)cc4ccccc4c3o2)cc1 ; c1ccc(-c2nc3c(o2)c(-c2ccc(N(c4ccc5c(c4)sc4ccccc45)c4ccccc4-c4ccccc4)cc2)cc2ccccc23)cc1]

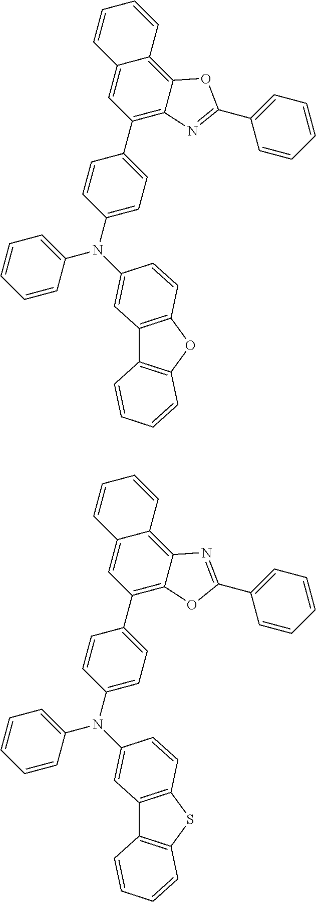 [SMILES: c1ccc(-c2nc3c(-c4ccc(N(c5ccccc5)c5ccc6oc7ccccc7c6c5)cc4)cc4ccccc4c3o2)cc1 ; c1ccc(-c2nc3c(o2)c(-c2ccc(N(c4ccccc4)c4ccc5sc6ccccc6c5c4)cc2)cc2ccccc23)cc1]

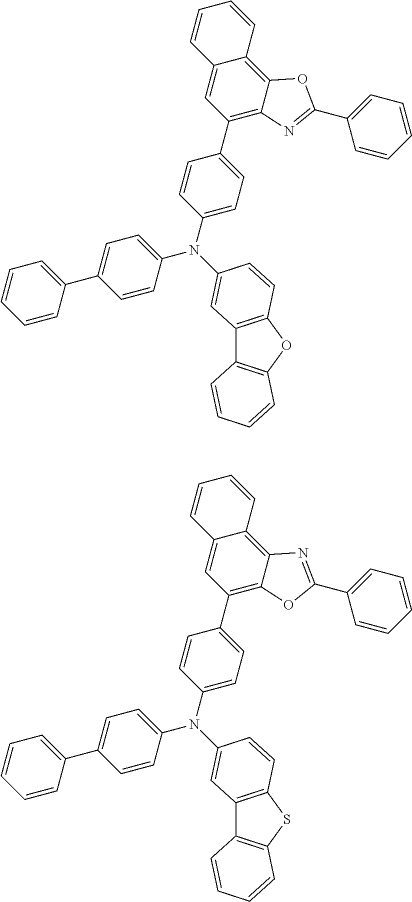 [SMILES: c1ccc(-c2ccc(N(c3ccc(-c4cc5ccccc5c5oc(-c6ccccc6)nc45)cc3)c3ccc4oc5ccccc5c4c3)cc2)cc1 ; c1ccc(-c2ccc(N(c3ccc(-c4cc5ccccc5c5nc(-c6ccccc6)oc45)cc3)c3ccc4sc5ccccc5c4c3)cc2)cc1]

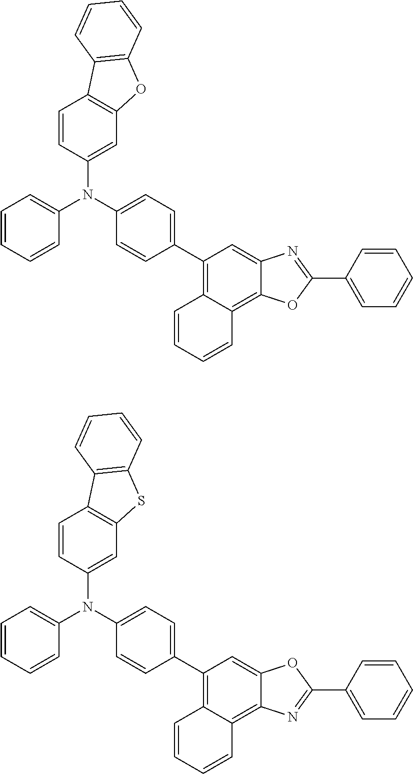 [SMILES: c1ccc(-c2nc3cc(-c4ccc(N(c5ccccc5)c5ccc6c(c5)oc5ccccc56)cc4)c4ccccc4c3o2)cc1 ; c1ccc(-c2nc3c(cc(-c4ccc(N(c5ccccc5)c5ccc6c(c5)sc5ccccc56)cc4)c4ccccc43)o2)cc1]

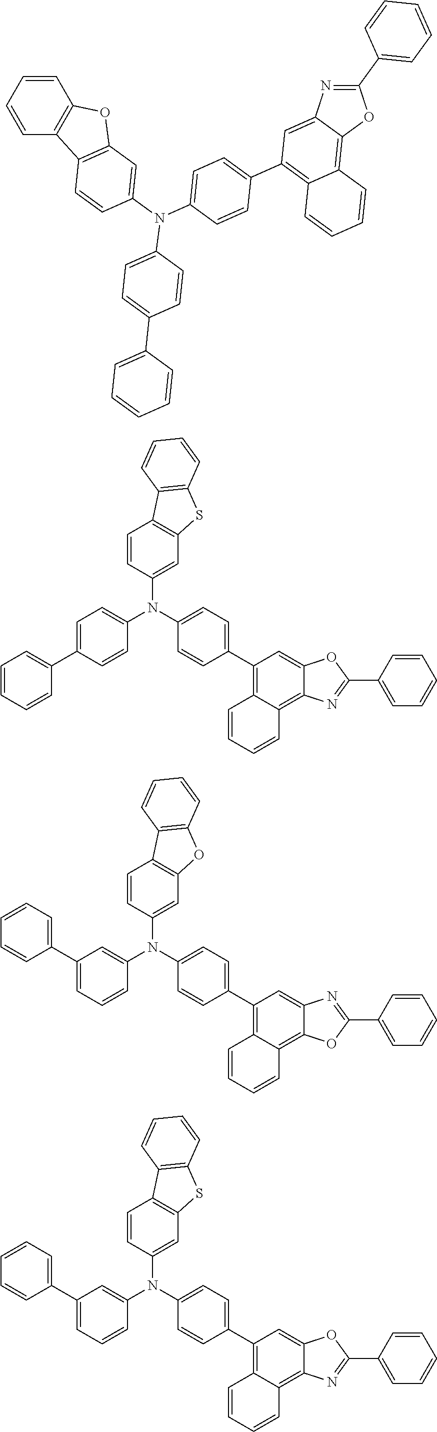

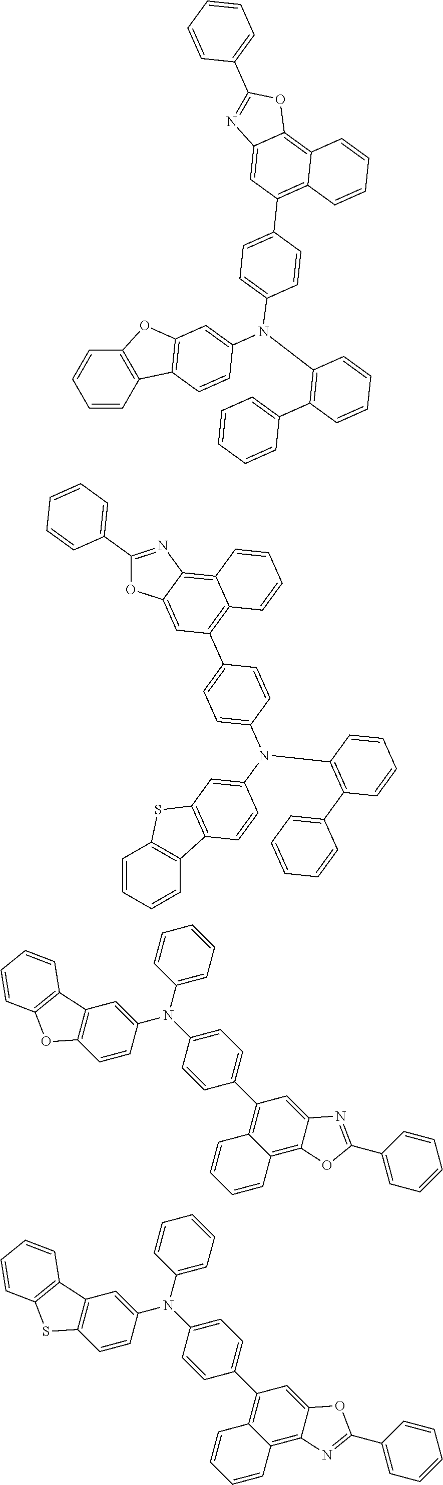

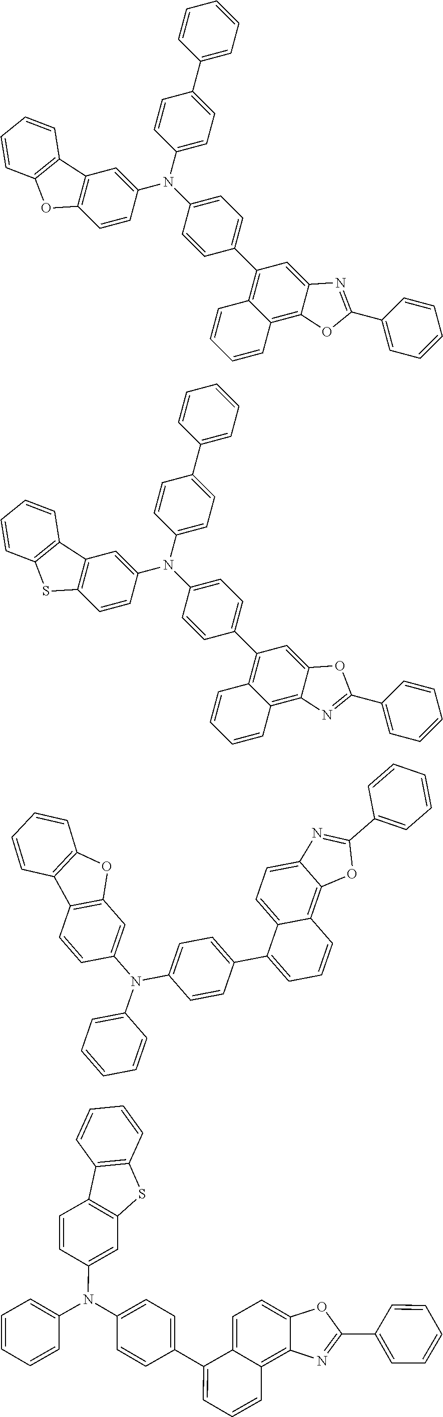

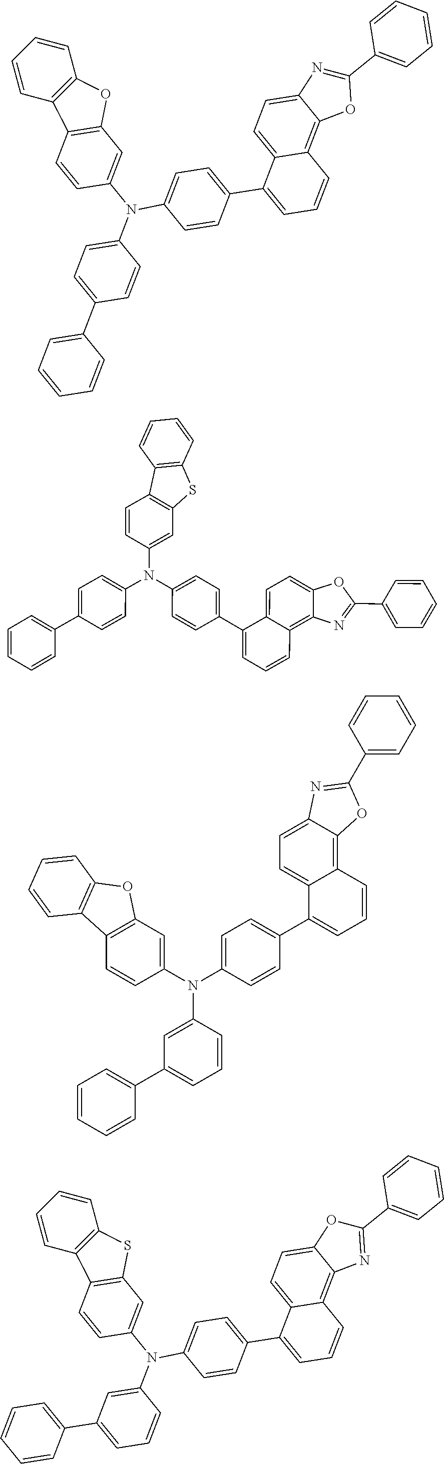 [SMILES: c1ccc(-c2ccc(N(c3ccc(-c4cccc5c4ccc4nc(-c6ccccc6)oc45)cc3)c3ccc4c(c3)oc3ccccc34)cc2)cc1 ; c1ccc(-c2ccc(N(c3ccc(-c4cccc5c4ccc4oc(-c6ccccc6)nc45)cc3)c3ccc4c(c3)sc3ccccc34)cc2)cc1 ; c1ccc(-c2cccc(N(c3ccc(-c4cccc5c4ccc4nc(-c6ccccc6)oc45)cc3)c3ccc4c(c3)oc3ccccc34)c2)cc1 ; c1ccc(-c2cccc(N(c3ccc(-c4cccc5c4ccc4oc(-c6ccccc6)nc45)cc3)c3ccc4c(c3)sc3ccccc34)c2)cc1]

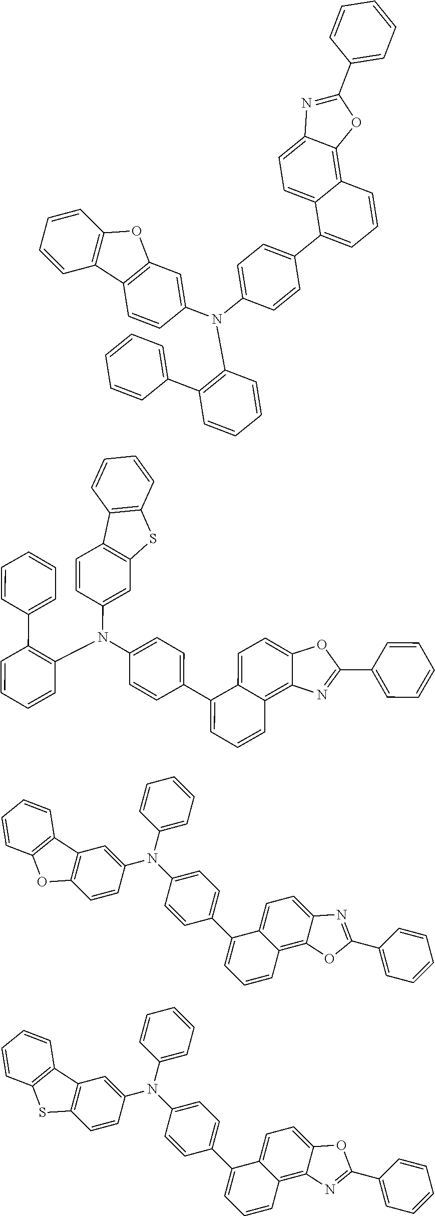 [SMILES: c1ccc(-c2nc3ccc4c(-c5ccc(N(c6ccc7c(c6)oc6ccccc67)c6ccccc6-c6ccccc6)cc5)cccc4c3o2)cc1 ; c1ccc(-c2nc3c(ccc4c(-c5ccc(N(c6ccc7c(c6)sc6ccccc67)c6ccccc6-c6ccccc6)cc5)cccc43)o2)cc1 ; c1ccc(-c2nc3ccc4c(-c5ccc(N(c6ccccc6)c6ccc7oc8ccccc8c7c6)cc5)cccc4c3o2)cc1 ; c1ccc(-c2nc3c(ccc4c(-c5ccc(N(c6ccccc6)c6ccc7sc8ccccc8c7c6)cc5)cccc43)o2)cc1]

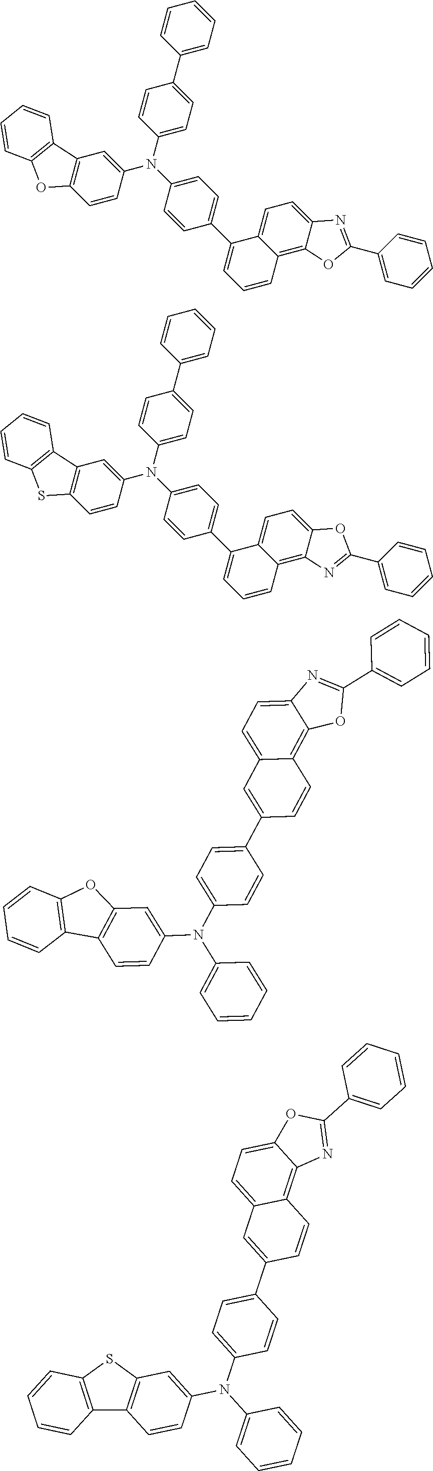

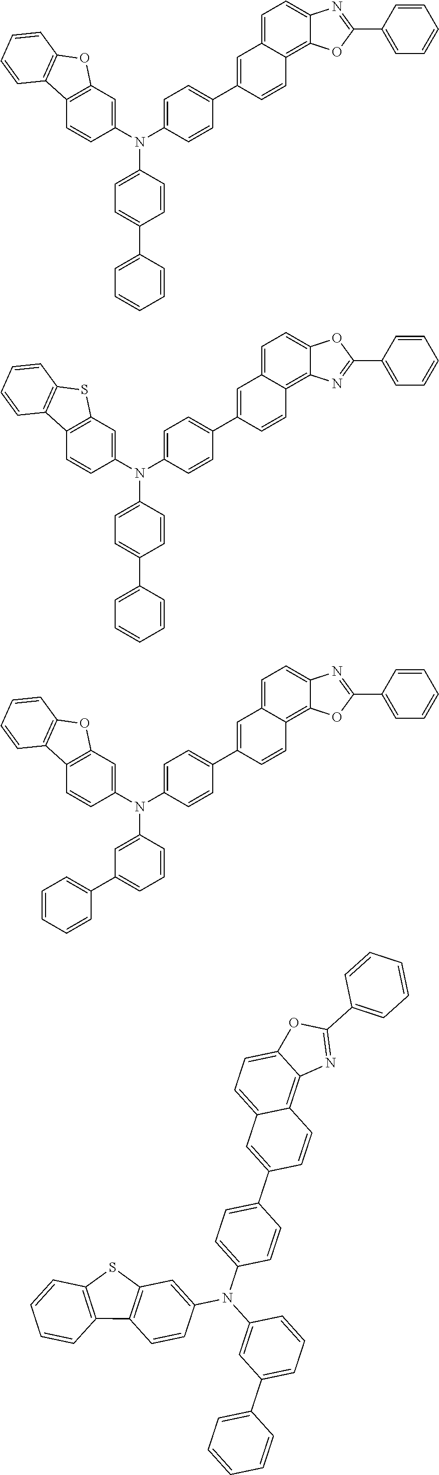 [SMILES: c1ccc(-c2ccc(N(c3ccc(-c4ccc5c(ccc6nc(-c7ccccc7)oc65)c4)cc3)c3ccc4c(c3)oc3ccccc34)cc2)cc1 ; c1ccc(-c2ccc(N(c3ccc(-c4ccc5c(ccc6oc(-c7ccccc7)nc65)c4)cc3)c3ccc4c(c3)sc3ccccc34)cc2)cc1 ; c1ccc(-c2cccc(N(c3ccc(-c4ccc5c(ccc6nc(-c7ccccc7)oc65)c4)cc3)c3ccc4c(c3)oc3ccccc34)c2)cc1 ; c1ccc(-c2cccc(N(c3ccc(-c4ccc5c(ccc6oc(-c7ccccc7)nc65)c4)cc3)c3ccc4c(c3)sc3ccccc34)c2)cc1]

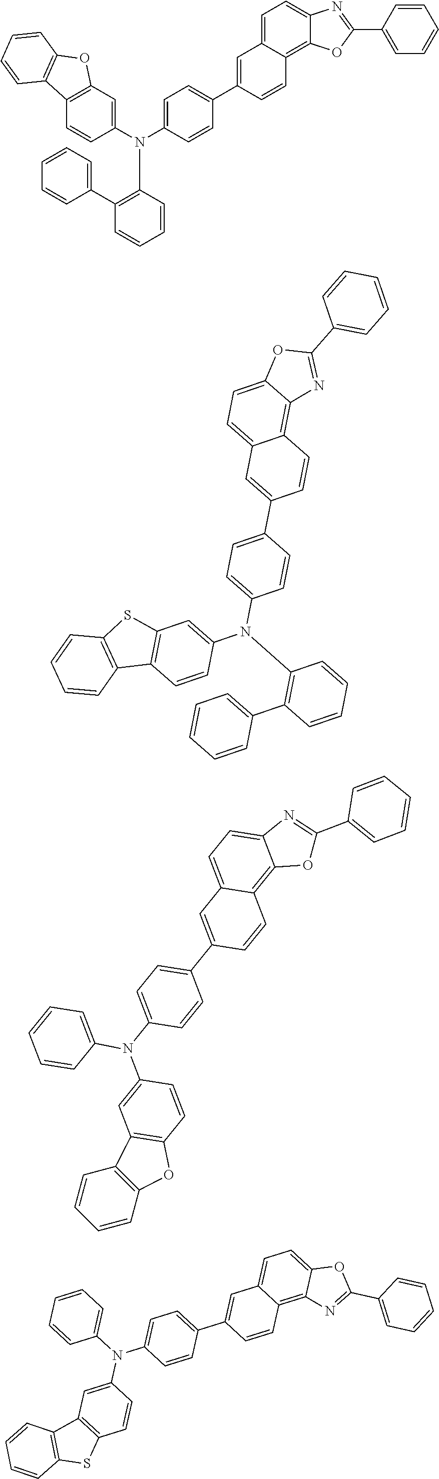 [SMILES: c1ccc(-c2nc3ccc4cc(-c5ccc(N(c6ccc7c(c6)oc6ccccc67)c6ccccc6-c6ccccc6)cc5)ccc4c3o2)cc1 ; c1ccc(-c2nc3c(ccc4cc(-c5ccc(N(c6ccc7c(c6)sc6ccccc67)c6ccccc6-c6ccccc6)cc5)ccc43)o2)cc1 ; c1ccc(-c2nc3ccc4cc(-c5ccc(N(c6ccccc6)c6ccc7oc8ccccc8c7c6)cc5)ccc4c3o2)cc1 ; c1ccc(-c2nc3c(ccc4cc(-c5ccc(N(c6ccccc6)c6ccc7sc8ccccc8c7c6)cc5)ccc43)o2)cc1]

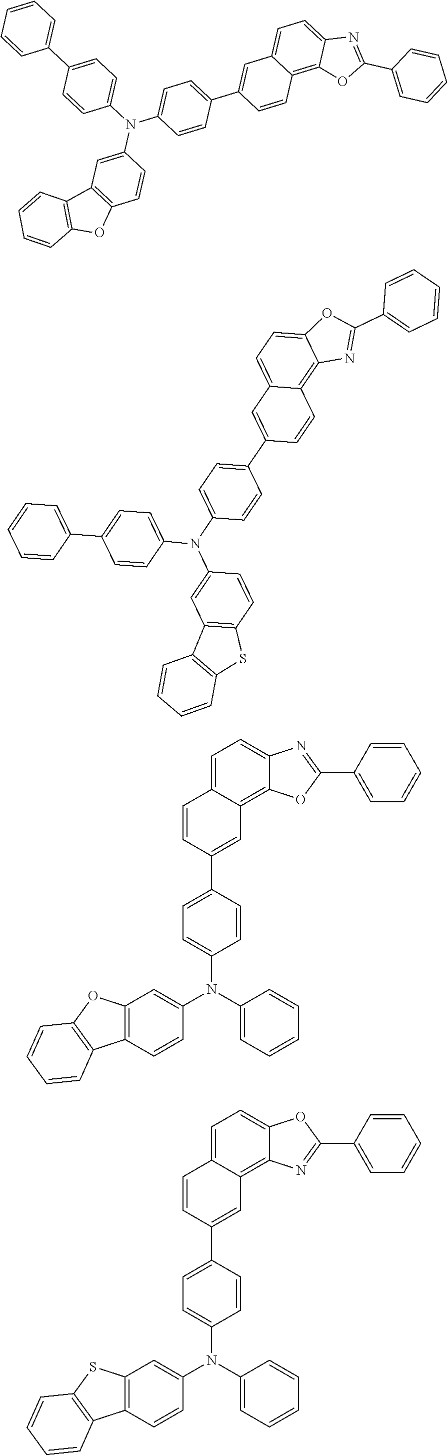 [SMILES: c1ccc(-c2ccc(N(c3ccc(-c4ccc5c(ccc6nc(-c7ccccc7)oc65)c4)cc3)c3ccc4oc5ccccc5c4c3)cc2)cc1 ; c1ccc(-c2ccc(N(c3ccc(-c4ccc5c(ccc6oc(-c7ccccc7)nc65)c4)cc3)c3ccc4sc5ccccc5c4c3)cc2)cc1 ; c1ccc(-c2nc3ccc4ccc(-c5ccc(N(c6ccccc6)c6ccc7c(c6)oc6ccccc67)cc5)cc4c3o2)cc1 ; c1ccc(-c2nc3c(ccc4ccc(-c5ccc(N(c6ccccc6)c6ccc7c(c6)sc6ccccc67)cc5)cc43)o2)cc1]

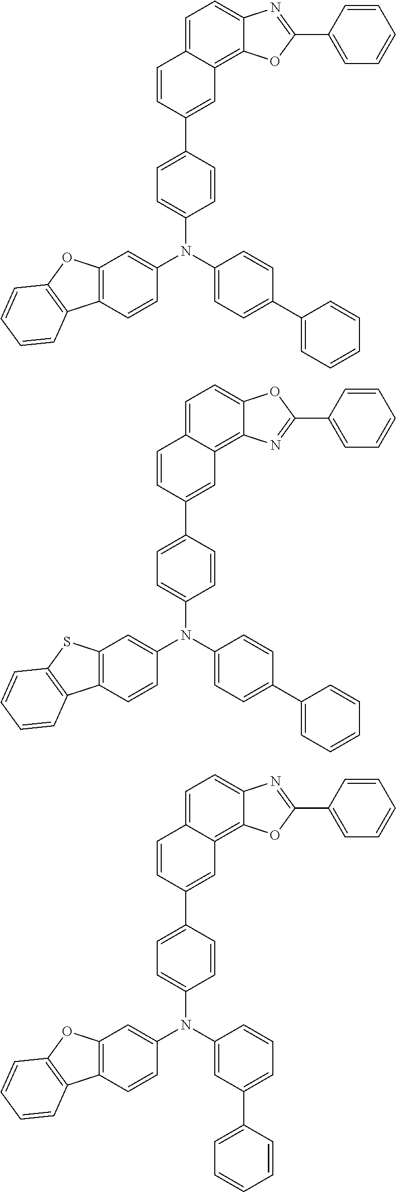

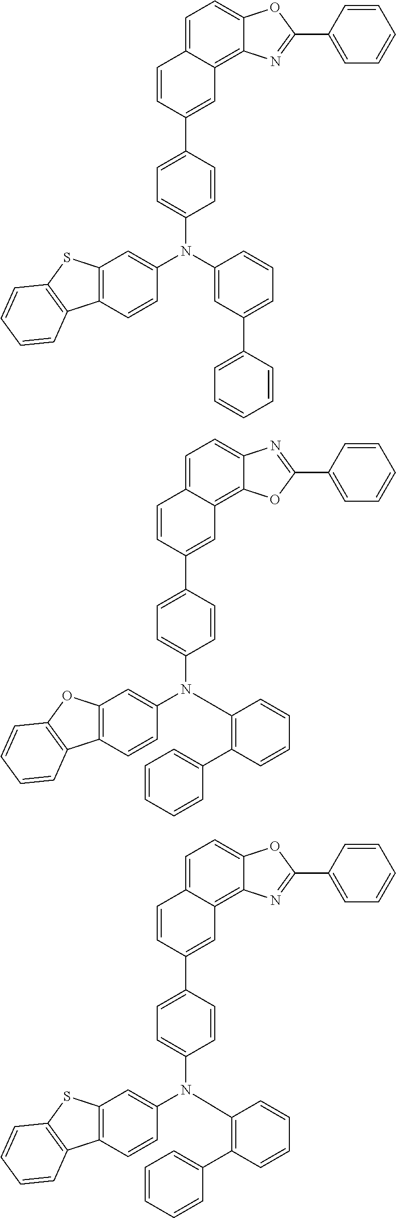

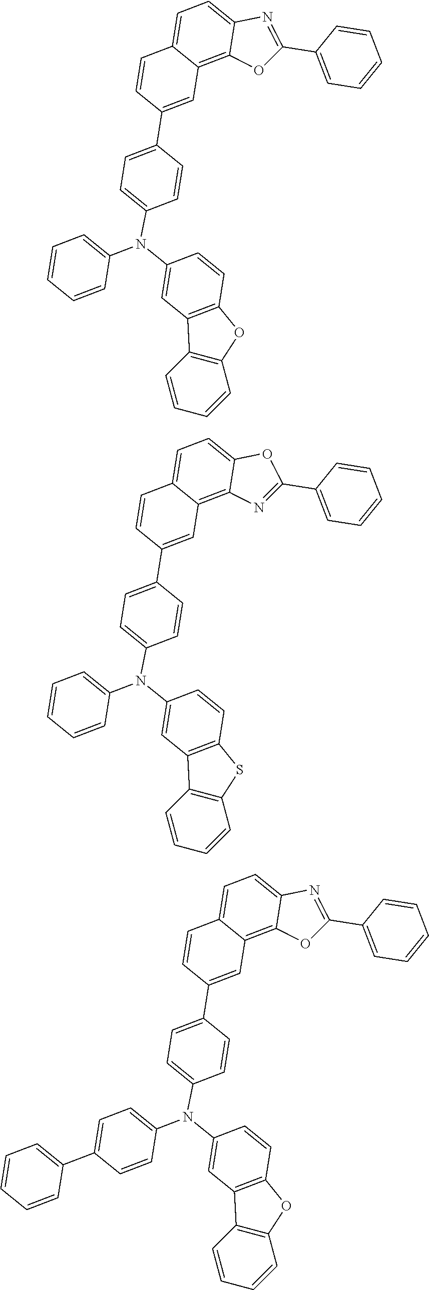

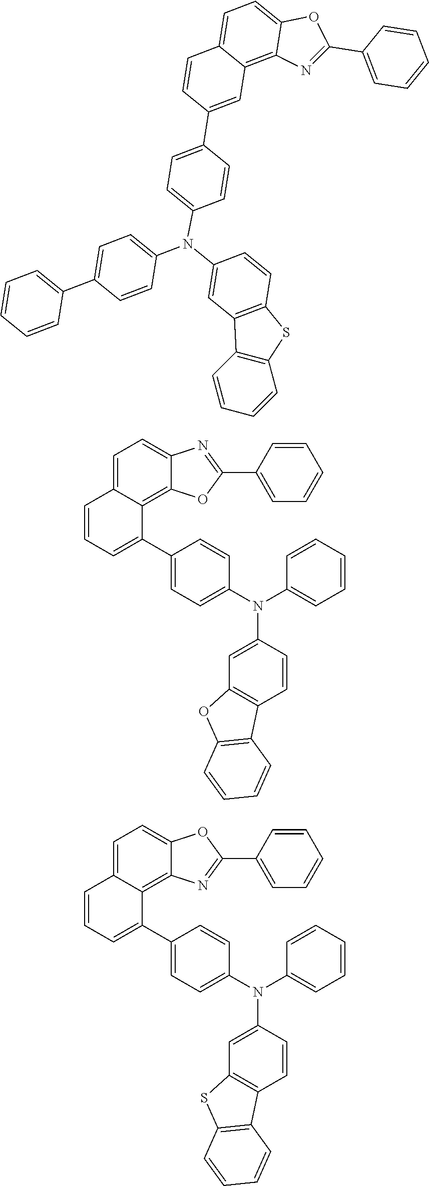

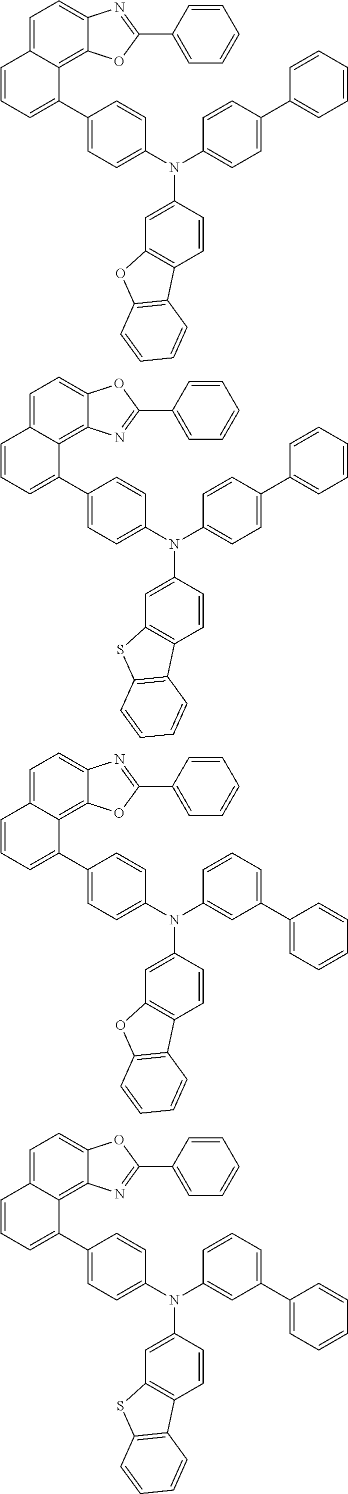

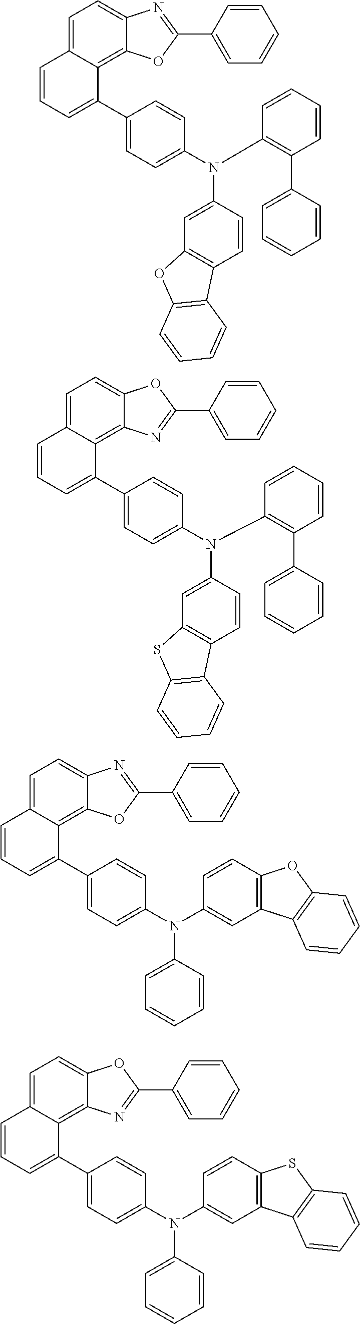

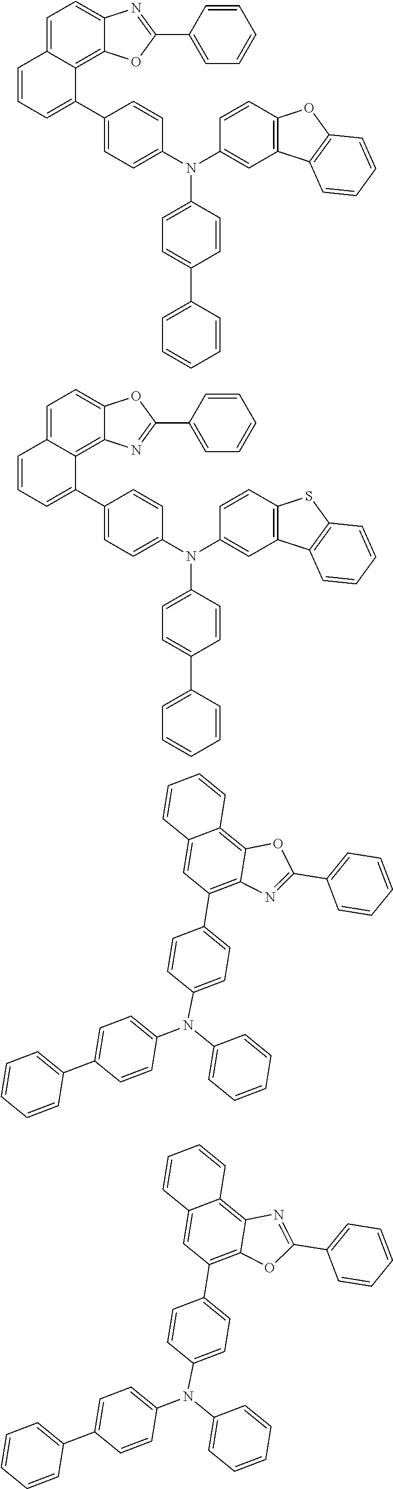

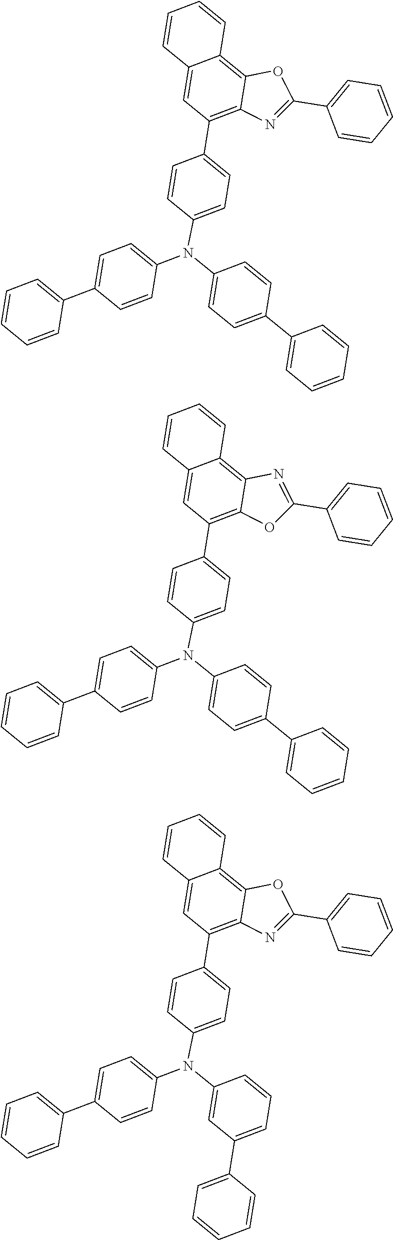 [SMILES: c1ccc(-c2ccc(N(c3ccc(-c4ccccc4)cc3)c3ccc(-c4cc5ccccc5c5oc(-c6ccccc6)nc45)cc3)cc2)cc1 ; c1ccc(-c2ccc(N(c3ccc(-c4ccccc4)cc3)c3ccc(-c4cc5ccccc5c5nc(-c6ccccc6)oc45)cc3)cc2)cc1 ; c1ccc(-c2ccc(N(c3ccc(-c4cc5ccccc5c5oc(-c6ccccc6)nc45)cc3)c3cccc(-c4ccccc4)c3)cc2)cc1]

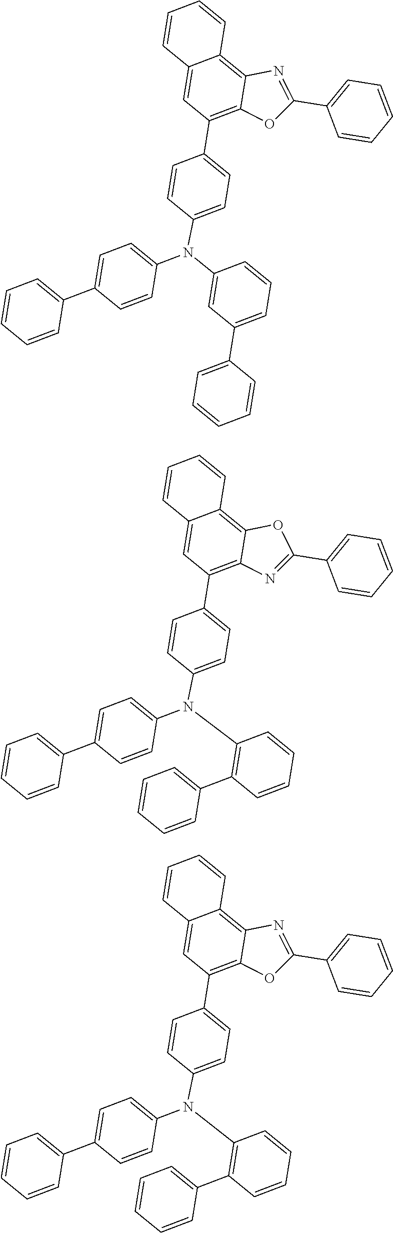

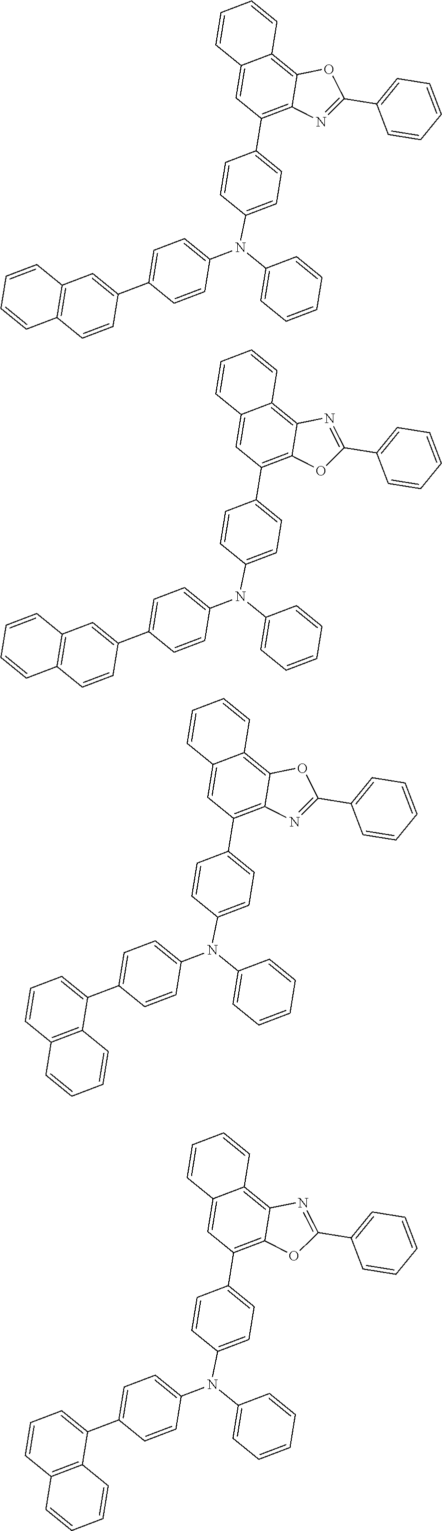

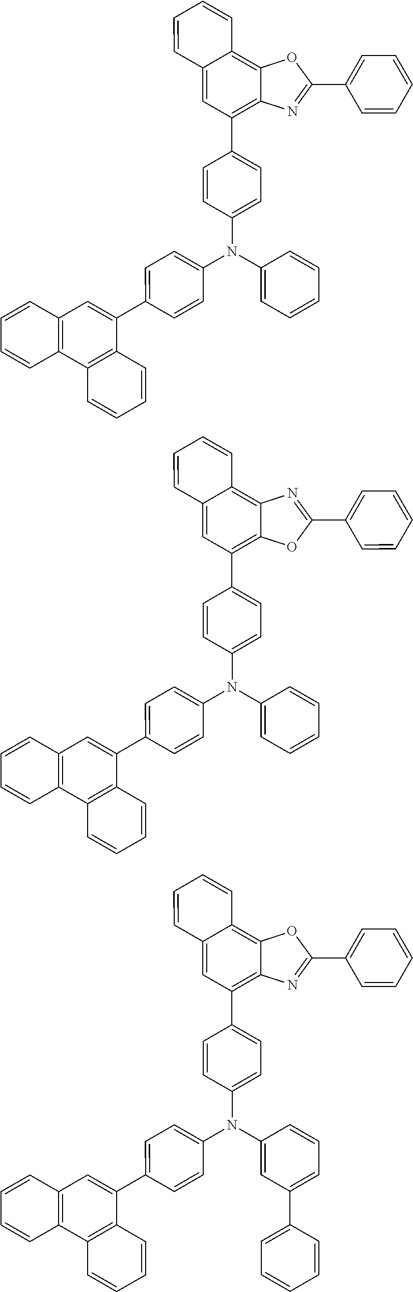 [SMILES: c1ccc(-c2nc3c(-c4ccc(N(c5ccccc5)c5ccc(-c6cc7ccccc7c7ccccc67)cc5)cc4)cc4ccccc4c3o2)cc1 ; c1ccc(-c2nc3c(o2)c(-c2ccc(N(c4ccccc4)c4ccc(-c5cc6ccccc6c6ccccc56)cc4)cc2)cc2ccccc23)cc1 ; c1ccc(-c2cccc(N(c3ccc(-c4cc5ccccc5c5ccccc45)cc3)c3ccc(-c4cc5ccccc5c5oc(-c6ccccc6)nc45)cc3)c2)cc1]

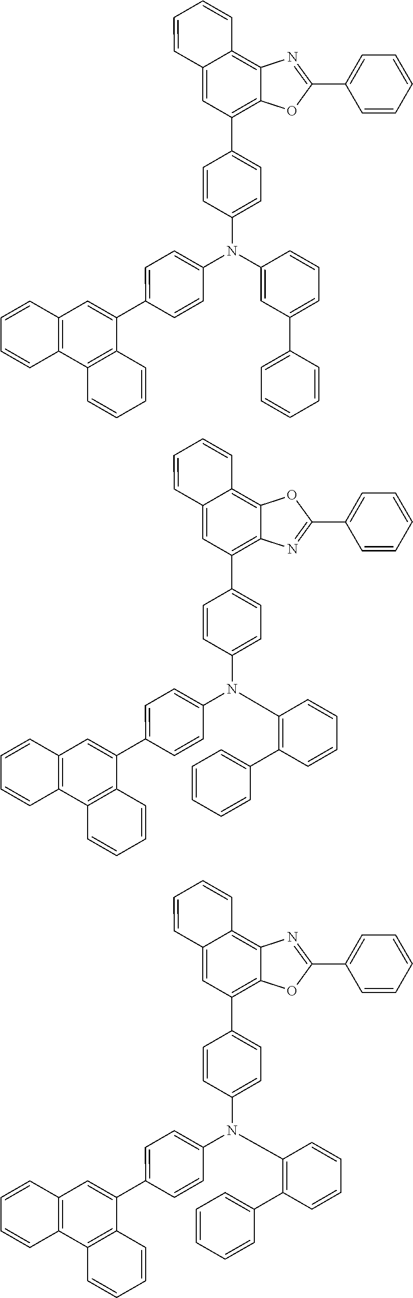 [SMILES: c1ccc(-c2cccc(N(c3ccc(-c4cc5ccccc5c5ccccc45)cc3)c3ccc(-c4cc5ccccc5c5nc(-c6ccccc6)oc45)cc3)c2)cc1 ; c1ccc(-c2nc3c(-c4ccc(N(c5ccc(-c6cc7ccccc7c7ccccc67)cc5)c5ccccc5-c5ccccc5)cc4)cc4ccccc4c3o2)cc1 ; c1ccc(-c2nc3c(o2)c(-c2ccc(N(c4ccc(-c5cc6ccccc6c6ccccc56)cc4)c4ccccc4-c4ccccc4)cc2)cc2ccccc23)cc1]

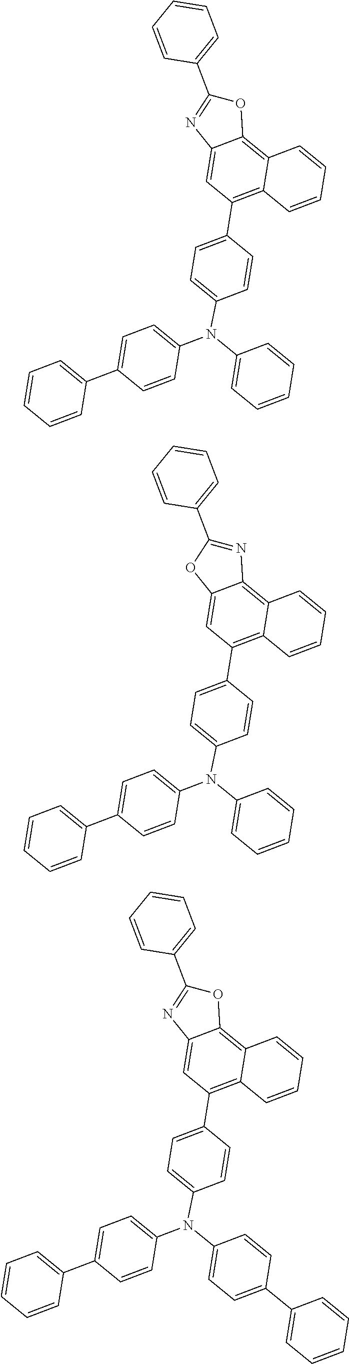 [SMILES: c1ccc(-c2ccc(N(c3ccccc3)c3ccc(-c4cc5nc(-c6ccccc6)oc5c5ccccc45)cc3)cc2)cc1 ; c1ccc(-c2ccc(N(c3ccccc3)c3ccc(-c4cc5oc(-c6ccccc6)nc5c5ccccc45)cc3)cc2)cc1 ; c1ccc(-c2ccc(N(c3ccc(-c4ccccc4)cc3)c3ccc(-c4cc5nc(-c6ccccc6)oc5c5ccccc45)cc3)cc2)cc1]

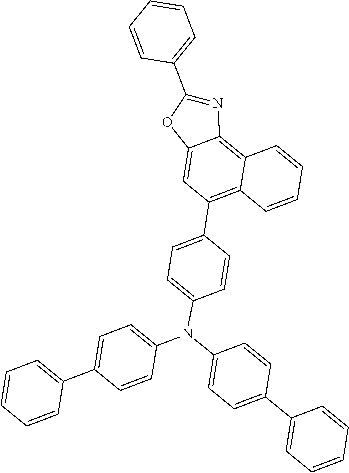 [SMILES: c1ccc(-c2ccc(N(c3ccc(-c4ccccc4)cc3)c3ccc(-c4cc5oc(-c6ccccc6)nc5c5ccccc45)cc3)cc2)cc1]

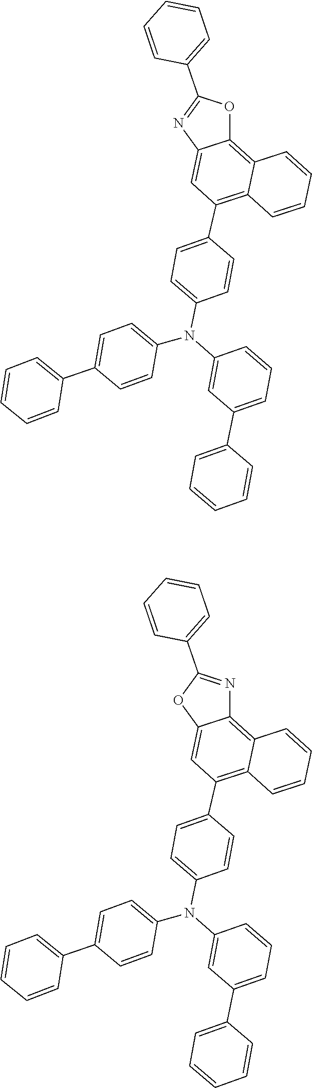 [SMILES: c1ccc(-c2ccc(N(c3ccc(-c4cc5nc(-c6ccccc6)oc5c5ccccc45)cc3)c3cccc(-c4ccccc4)c3)cc2)cc1 ; c1ccc(-c2ccc(N(c3ccc(-c4cc5oc(-c6ccccc6)nc5c5ccccc45)cc3)c3cccc(-c4ccccc4)c3)cc2)cc1]

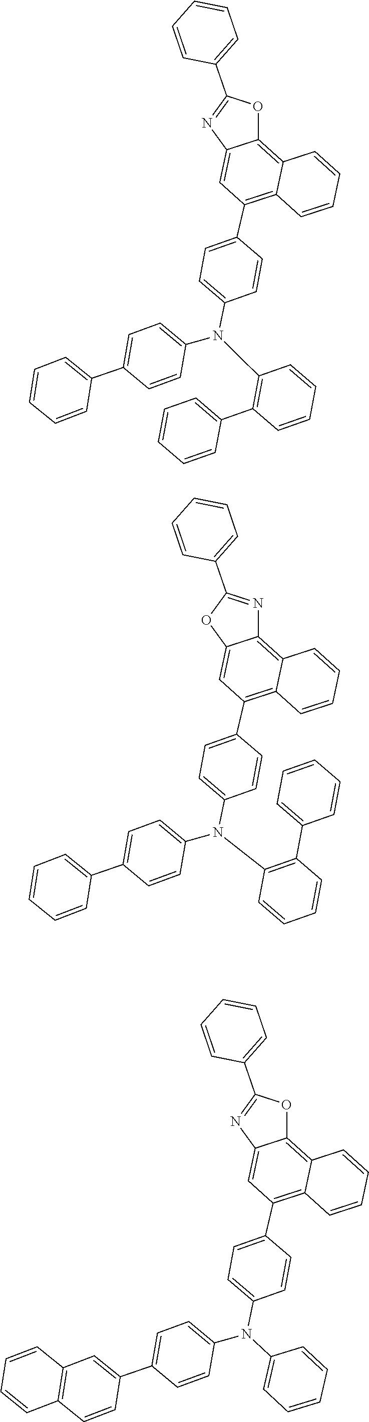

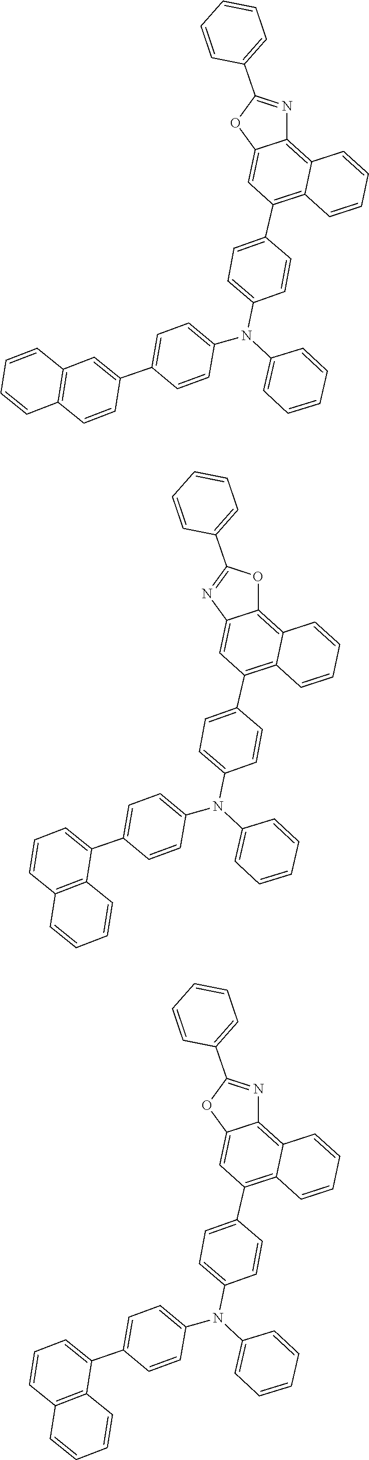 [SMILES: c1ccc(-c2nc3c(cc(-c4ccc(N(c5ccccc5)c5ccc(-c6ccc7ccccc7c6)cc5)cc4)c4ccccc43)o2)cc1 ; c1ccc(-c2nc3cc(-c4ccc(N(c5ccccc5)c5ccc(-c6cccc7ccccc67)cc5)cc4)c4ccccc4c3o2)cc1 ; c1ccc(-c2nc3c(cc(-c4ccc(N(c5ccccc5)c5ccc(-c6cccc7ccccc67)cc5)cc4)c4ccccc43)o2)cc1]

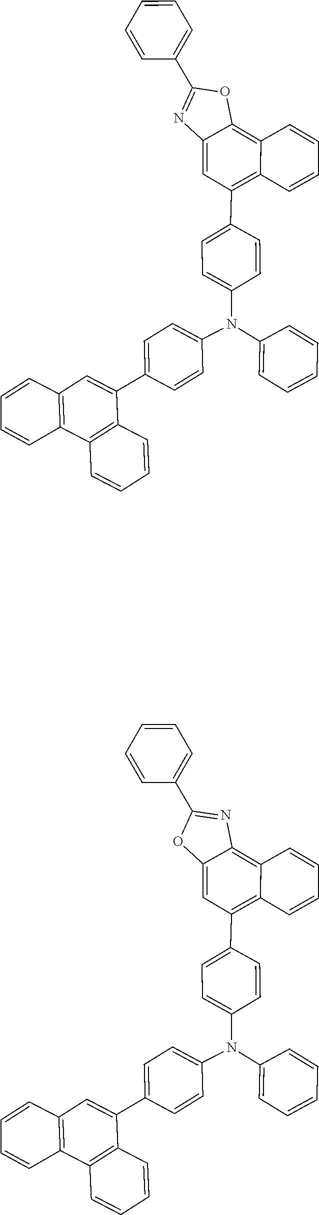 [SMILES: c1ccc(-c2nc3cc(-c4ccc(N(c5ccccc5)c5ccc(-c6cc7ccccc7c7ccccc67)cc5)cc4)c4ccccc4c3o2)cc1 ; c1ccc(-c2nc3c(cc(-c4ccc(N(c5ccccc5)c5ccc(-c6cc7ccccc7c7ccccc67)cc5)cc4)c4ccccc43)o2)cc1]

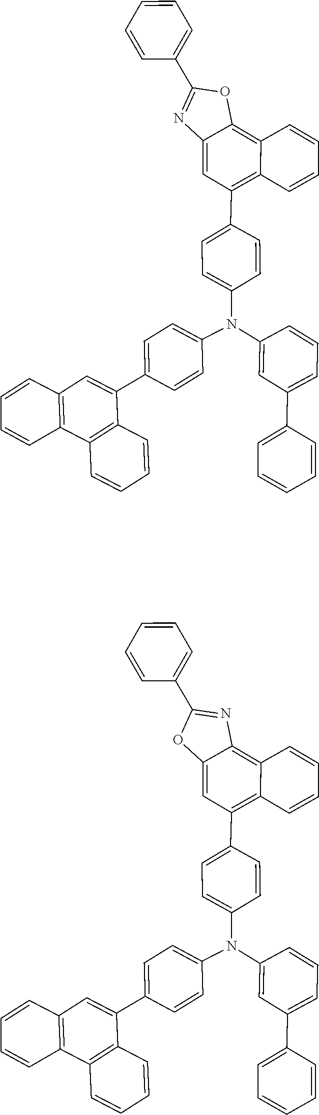 [SMILES: c1ccc(-c2cccc(N(c3ccc(-c4cc5ccccc5c5ccccc45)cc3)c3ccc(-c4cc5nc(-c6ccccc6)oc5c5ccccc45)cc3)c2)cc1 ; c1ccc(-c2cccc(N(c3ccc(-c4cc5ccccc5c5ccccc45)cc3)c3ccc(-c4cc5oc(-c6ccccc6)nc5c5ccccc45)cc3)c2)cc1]

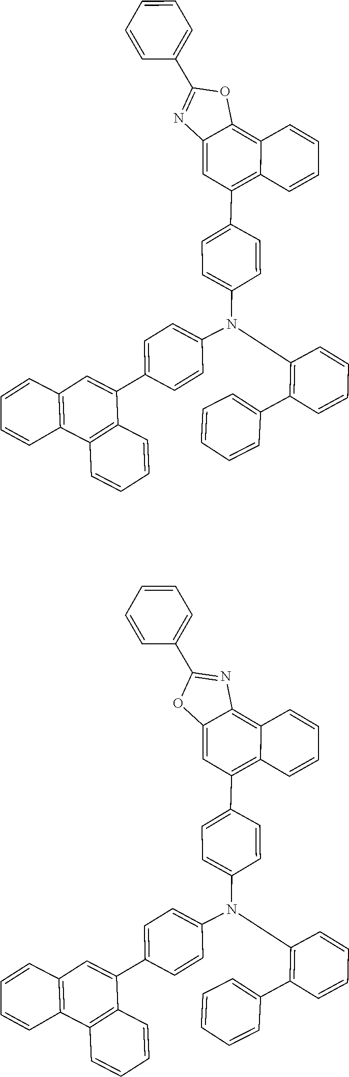 [SMILES: c1ccc(-c2nc3cc(-c4ccc(N(c5ccc(-c6cc7ccccc7c7ccccc67)cc5)c5ccccc5-c5ccccc5)cc4)c4ccccc4c3o2)cc1 ; c1ccc(-c2nc3c(cc(-c4ccc(N(c5ccc(-c6cc7ccccc7c7ccccc67)cc5)c5ccccc5-c5ccccc5)cc4)c4ccccc43)o2)cc1]

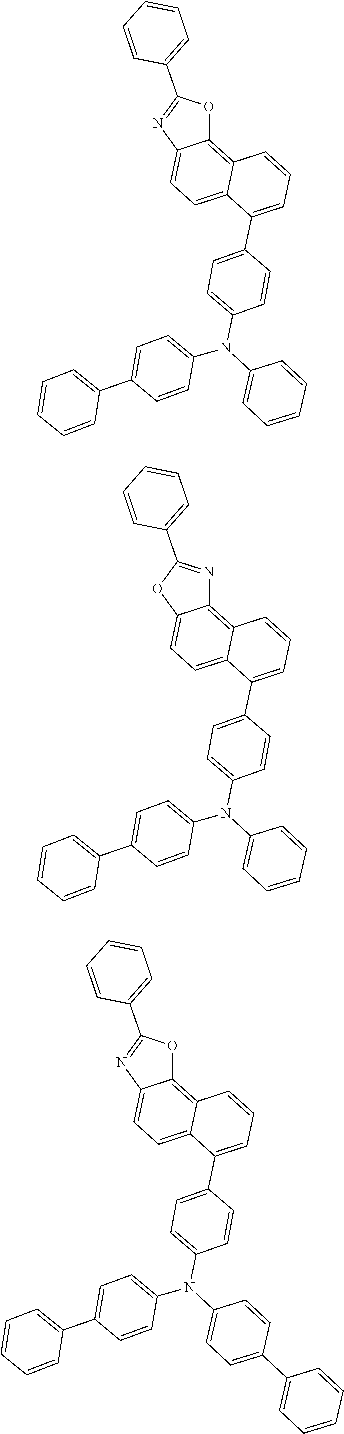 [SMILES: c1ccc(-c2ccc(N(c3ccccc3)c3ccc(-c4cccc5c4ccc4nc(-c6ccccc6)oc45)cc3)cc2)cc1 ; c1ccc(-c2ccc(N(c3ccccc3)c3ccc(-c4cccc5c4ccc4oc(-c6ccccc6)nc45)cc3)cc2)cc1 ; c1ccc(-c2ccc(N(c3ccc(-c4ccccc4)cc3)c3ccc(-c4cccc5c4ccc4nc(-c6ccccc6)oc45)cc3)cc2)cc1]

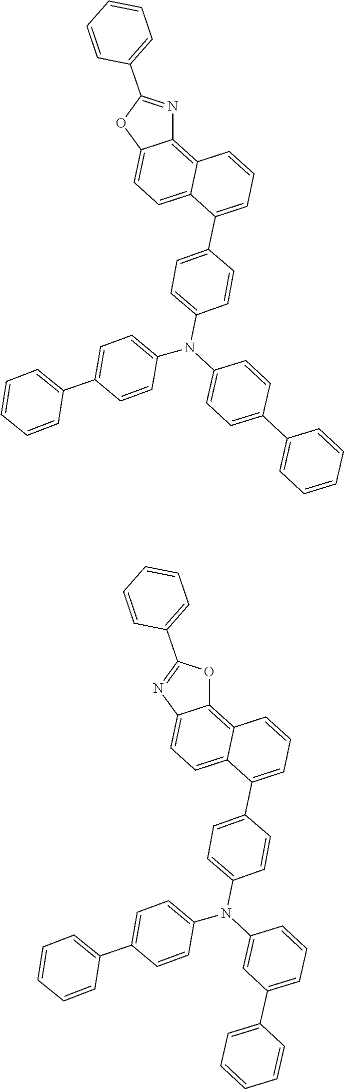 [SMILES: c1ccc(-c2ccc(N(c3ccc(-c4ccccc4)cc3)c3ccc(-c4cccc5c4ccc4oc(-c6ccccc6)nc45)cc3)cc2)cc1 ; c1ccc(-c2ccc(N(c3ccc(-c4cccc5c4ccc4nc(-c6ccccc6)oc45)cc3)c3cccc(-c4ccccc4)c3)cc2)cc1]

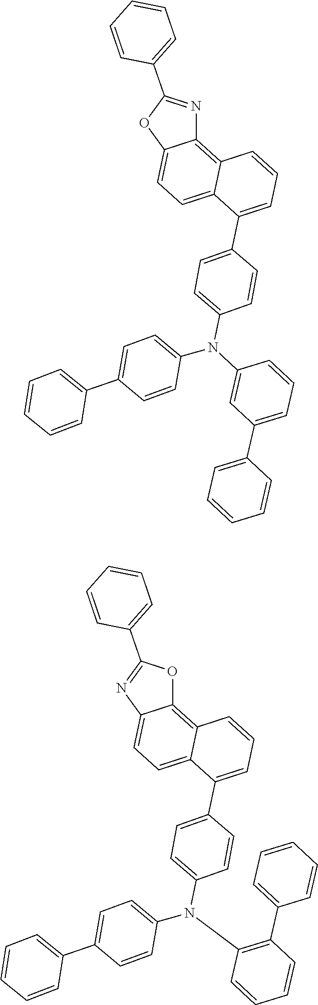 [SMILES: c1ccc(-c2ccc(N(c3ccc(-c4cccc5c4ccc4oc(-c6ccccc6)nc45)cc3)c3cccc(-c4ccccc4)c3)cc2)cc1 ; c1ccc(-c2ccc(N(c3ccc(-c4cccc5c4ccc4nc(-c6ccccc6)oc45)cc3)c3ccccc3-c3ccccc3)cc2)cc1]

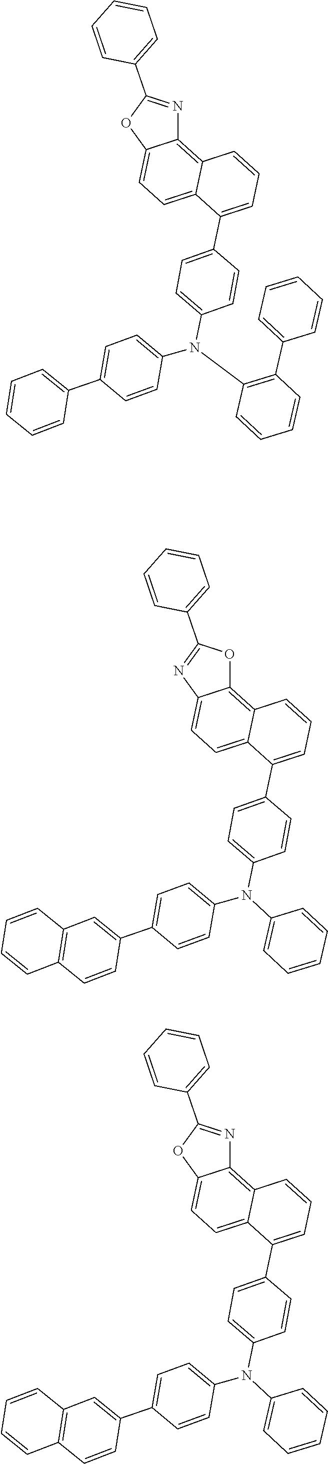 [SMILES: c1ccc(-c2ccc(N(c3ccc(-c4cccc5c4ccc4oc(-c6ccccc6)nc45)cc3)c3ccccc3-c3ccccc3)cc2)cc1 ; c1ccc(-c2nc3ccc4c(-c5ccc(N(c6ccccc6)c6ccc(-c7ccc8ccccc8c7)cc6)cc5)cccc4c3o2)cc1 ; c1ccc(-c2nc3c(ccc4c(-c5ccc(N(c6ccccc6)c6ccc(-c7ccc8ccccc8c7)cc6)cc5)cccc43)o2)cc1]

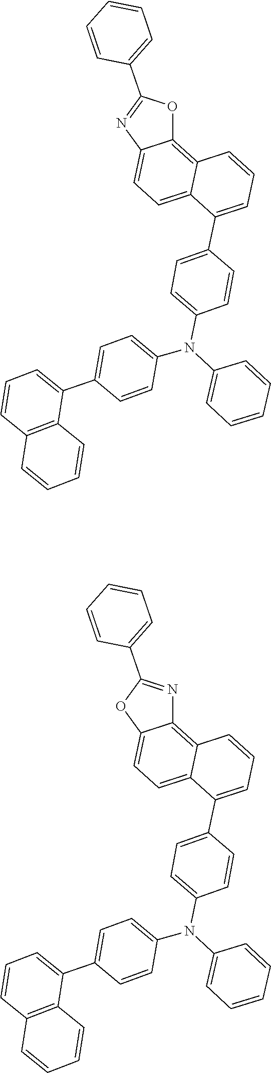 [SMILES: c1ccc(-c2nc3ccc4c(-c5ccc(N(c6ccccc6)c6ccc(-c7cccc8ccccc78)cc6)cc5)cccc4c3o2)cc1 ; c1ccc(-c2nc3c(ccc4c(-c5ccc(N(c6ccccc6)c6ccc(-c7cccc8ccccc78)cc6)cc5)cccc43)o2)cc1]

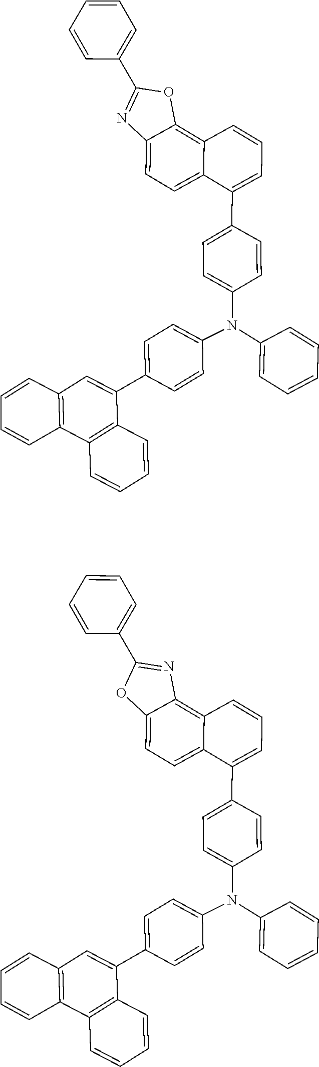 [SMILES: c1ccc(-c2nc3ccc4c(-c5ccc(N(c6ccccc6)c6ccc(-c7cc8ccccc8c8ccccc78)cc6)cc5)cccc4c3o2)cc1 ; c1ccc(-c2nc3c(ccc4c(-c5ccc(N(c6ccccc6)c6ccc(-c7cc8ccccc8c8ccccc78)cc6)cc5)cccc43)o2)cc1]

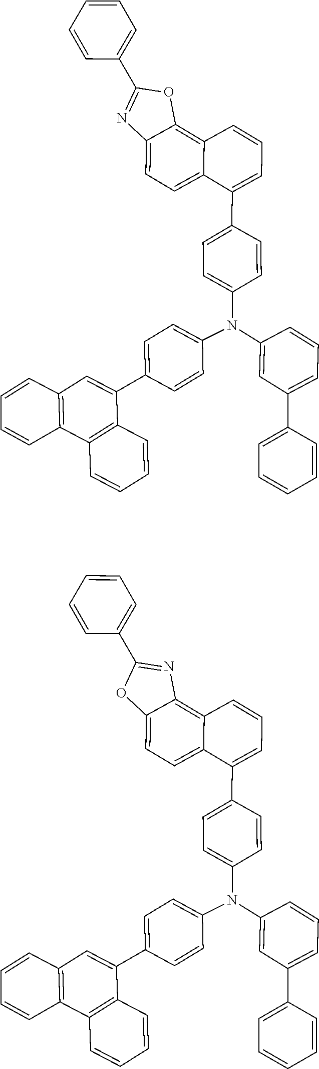 [SMILES: c1ccc(-c2cccc(N(c3ccc(-c4cc5ccccc5c5ccccc45)cc3)c3ccc(-c4cccc5c4ccc4nc(-c6ccccc6)oc45)cc3)c2)cc1 ; c1ccc(-c2cccc(N(c3ccc(-c4cc5ccccc5c5ccccc45)cc3)c3ccc(-c4cccc5c4ccc4oc(-c6ccccc6)nc45)cc3)c2)cc1]

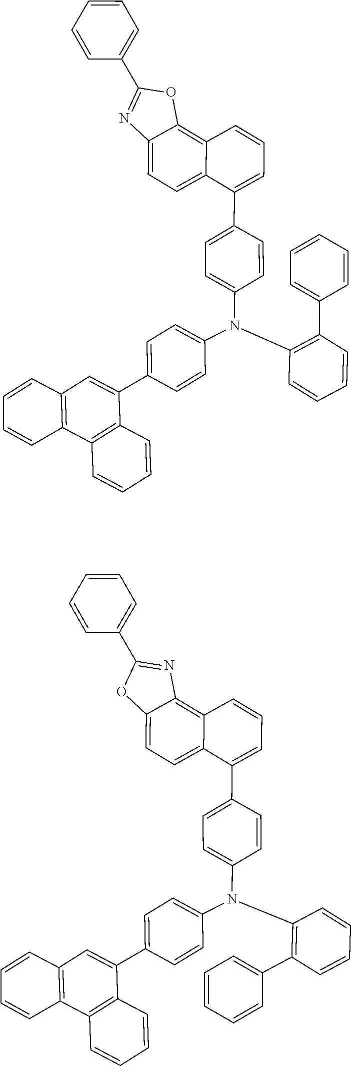 [SMILES: c1ccc(-c2nc3ccc4c(-c5ccc(N(c6ccc(-c7cc8ccccc8c8ccccc78)cc6)c6ccccc6-c6ccccc6)cc5)cccc4c3o2)cc1 ; c1ccc(-c2nc3c(ccc4c(-c5ccc(N(c6ccc(-c7cc8ccccc8c8ccccc78)cc6)c6ccccc6-c6ccccc6)cc5)cccc43)o2)cc1]

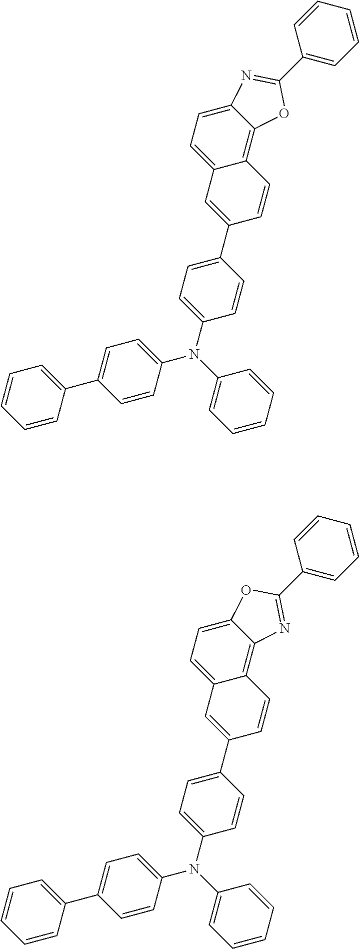 [SMILES: c1ccc(-c2ccc(N(c3ccccc3)c3ccc(-c4ccc5c(ccc6nc(-c7ccccc7)oc65)c4)cc3)cc2)cc1 ; c1ccc(-c2ccc(N(c3ccccc3)c3ccc(-c4ccc5c(ccc6oc(-c7ccccc7)nc65)c4)cc3)cc2)cc1]

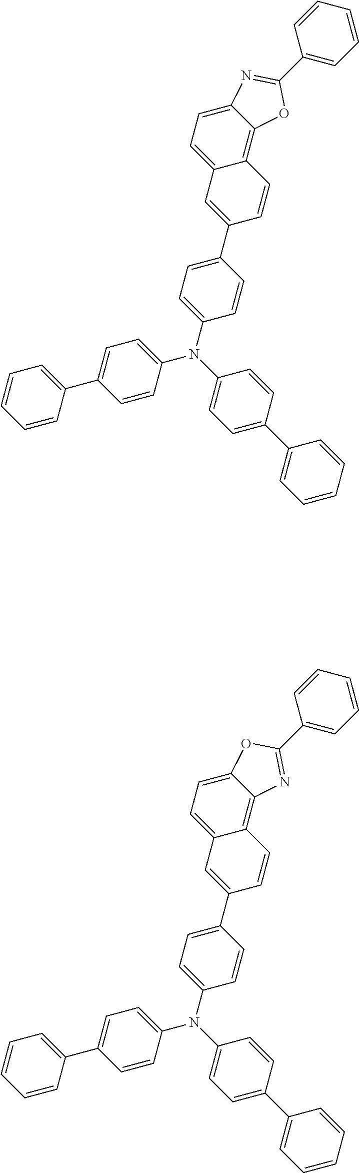 [SMILES: c1ccc(-c2ccc(N(c3ccc(-c4ccccc4)cc3)c3ccc(-c4ccc5c(ccc6nc(-c7ccccc7)oc65)c4)cc3)cc2)cc1 ; c1ccc(-c2ccc(N(c3ccc(-c4ccccc4)cc3)c3ccc(-c4ccc5c(ccc6oc(-c7ccccc7)nc65)c4)cc3)cc2)cc1]

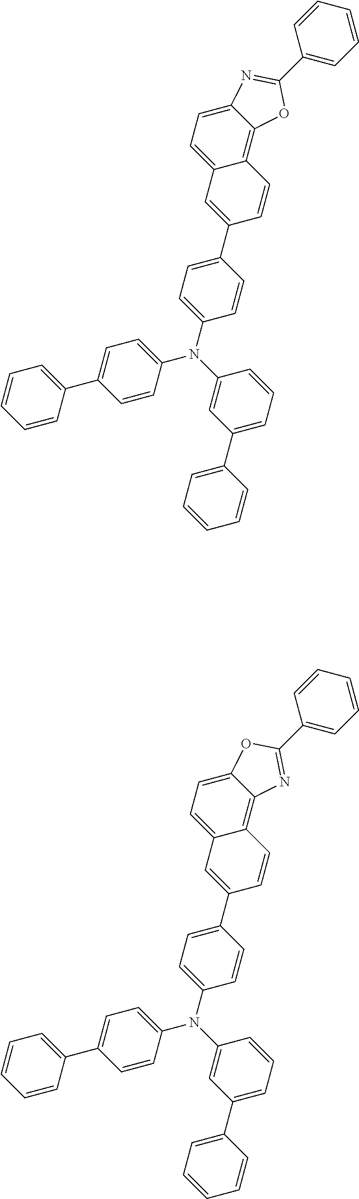 [SMILES: c1ccc(-c2ccc(N(c3ccc(-c4ccc5c(ccc6nc(-c7ccccc7)oc65)c4)cc3)c3cccc(-c4ccccc4)c3)cc2)cc1 ; c1ccc(-c2ccc(N(c3ccc(-c4ccc5c(ccc6oc(-c7ccccc7)nc65)c4)cc3)c3cccc(-c4ccccc4)c3)cc2)cc1]

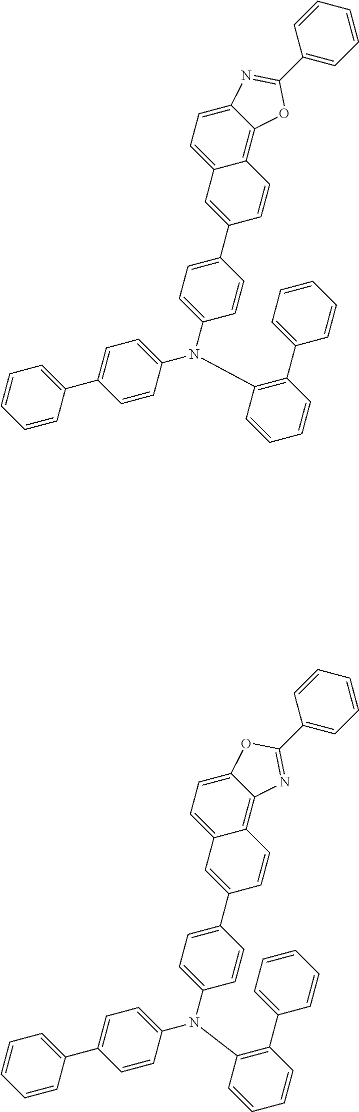 [SMILES: c1ccc(-c2ccc(N(c3ccc(-c4ccc5c(ccc6nc(-c7ccccc7)oc65)c4)cc3)c3ccccc3-c3ccccc3)cc2)cc1 ; c1ccc(-c2ccc(N(c3ccc(-c4ccc5c(ccc6oc(-c7ccccc7)nc65)c4)cc3)c3ccccc3-c3ccccc3)cc2)cc1]

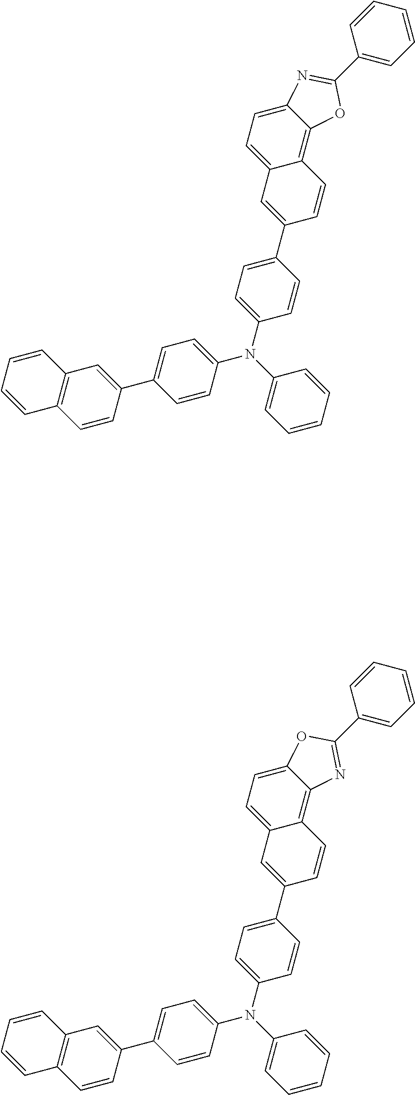 [SMILES: c1ccc(-c2nc3ccc4cc(-c5ccc(N(c6ccccc6)c6ccc(-c7ccc8ccccc8c7)cc6)cc5)ccc4c3o2)cc1 ; c1ccc(-c2nc3c(ccc4cc(-c5ccc(N(c6ccccc6)c6ccc(-c7ccc8ccccc8c7)cc6)cc5)ccc43)o2)cc1]

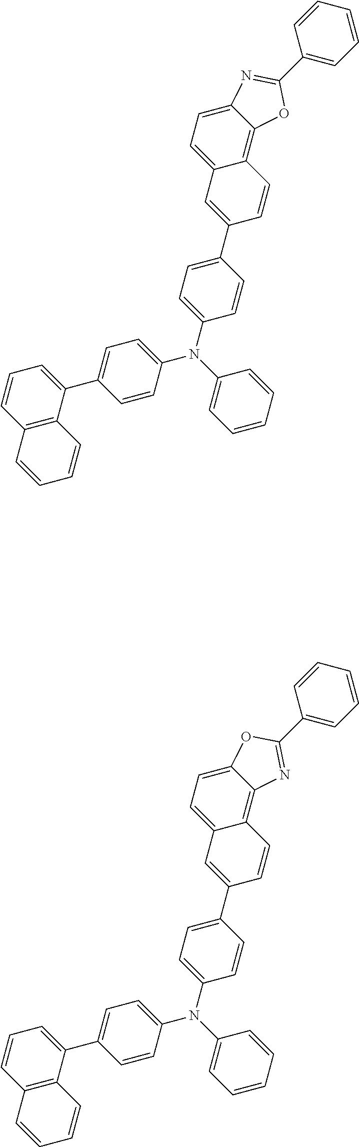 [SMILES: c1ccc(-c2nc3ccc4cc(-c5ccc(N(c6ccccc6)c6ccc(-c7cccc8ccccc78)cc6)cc5)ccc4c3o2)cc1 ; c1ccc(-c2nc3c(ccc4cc(-c5ccc(N(c6ccccc6)c6ccc(-c7cccc8ccccc78)cc6)cc5)ccc43)o2)cc1]

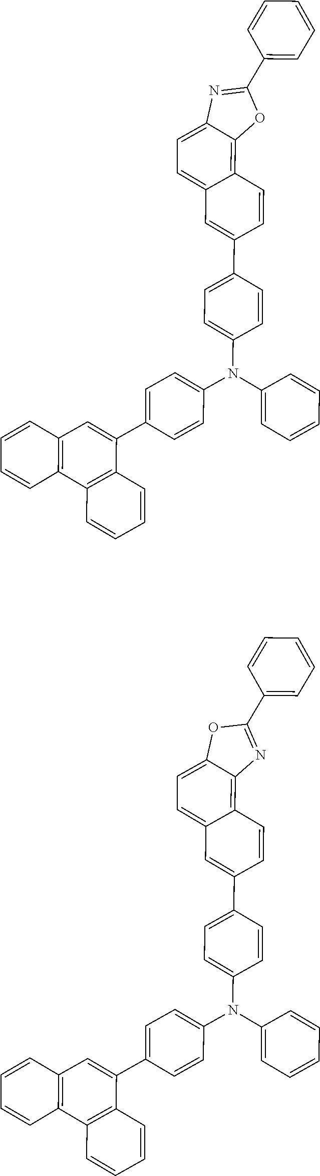 [SMILES: c1ccc(-c2nc3ccc4cc(-c5ccc(N(c6ccccc6)c6ccc(-c7cc8ccccc8c8ccccc78)cc6)cc5)ccc4c3o2)cc1 ; c1ccc(-c2nc3c(ccc4cc(-c5ccc(N(c6ccccc6)c6ccc(-c7cc8ccccc8c8ccccc78)cc6)cc5)ccc43)o2)cc1]

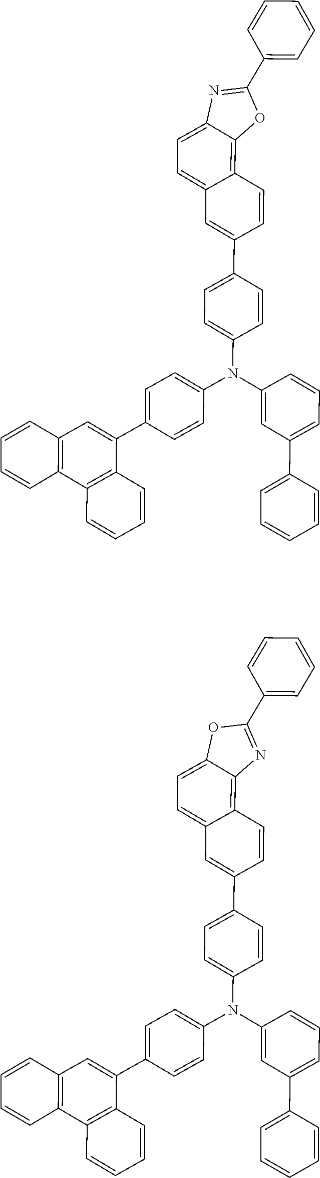 [SMILES: c1ccc(-c2cccc(N(c3ccc(-c4ccc5c(ccc6nc(-c7ccccc7)oc65)c4)cc3)c3ccc(-c4cc5ccccc5c5ccccc45)cc3)c2)cc1 ; c1ccc(-c2cccc(N(c3ccc(-c4ccc5c(ccc6oc(-c7ccccc7)nc65)c4)cc3)c3ccc(-c4cc5ccccc5c5ccccc45)cc3)c2)cc1]

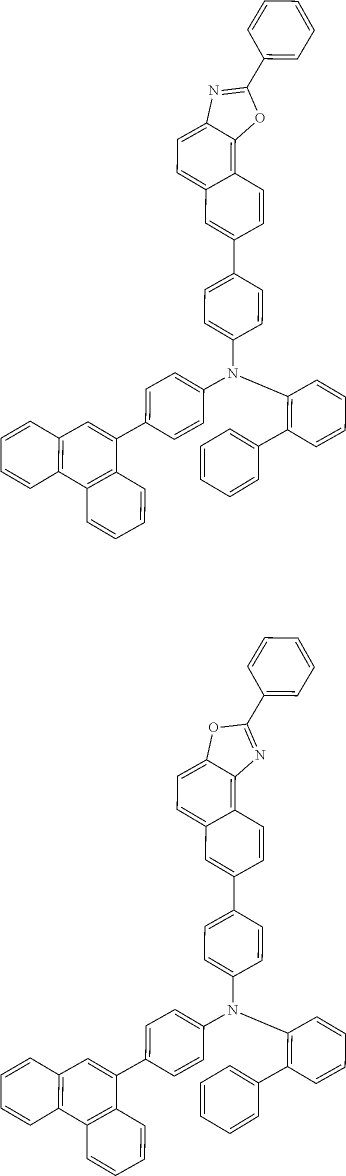 [SMILES: c1ccc(-c2nc3ccc4cc(-c5ccc(N(c6ccc(-c7cc8ccccc8c8ccccc78)cc6)c6ccccc6-c6ccccc6)cc5)ccc4c3o2)cc1 ; c1ccc(-c2nc3c(ccc4cc(-c5ccc(N(c6ccc(-c7cc8ccccc8c8ccccc78)cc6)c6ccccc6-c6ccccc6)cc5)ccc43)o2)cc1]

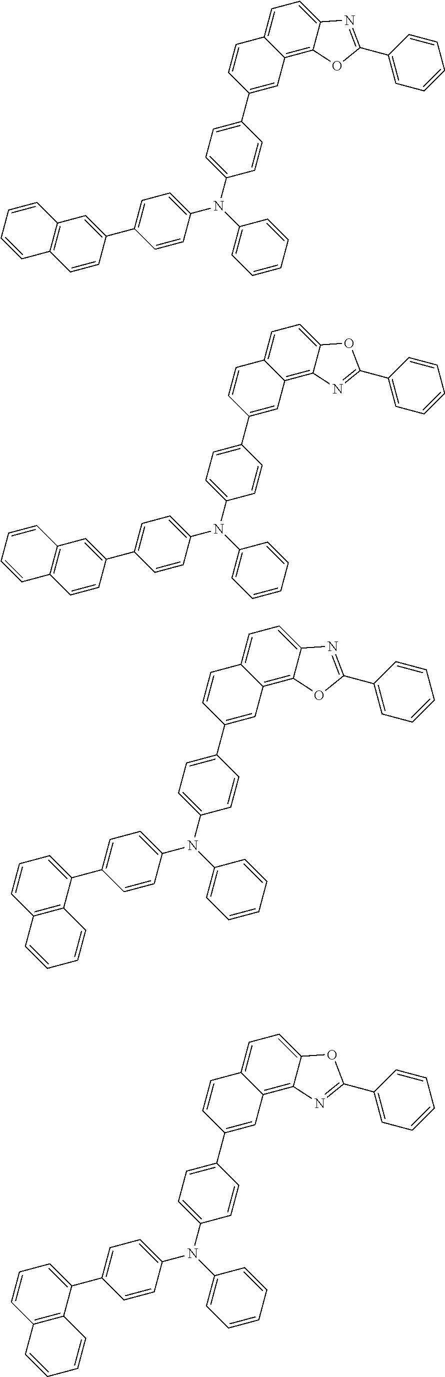

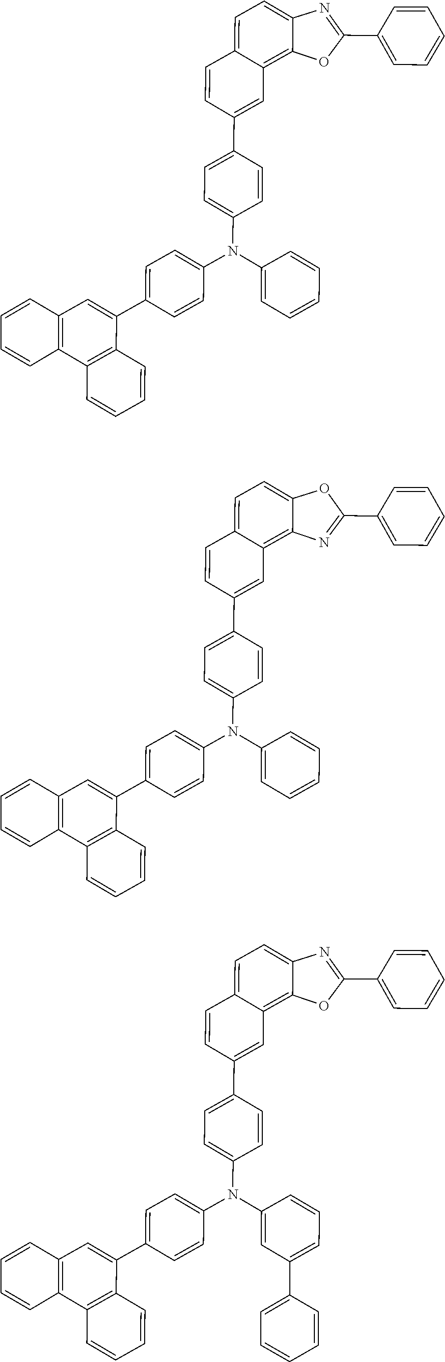 [SMILES: c1ccc(-c2nc3ccc4ccc(-c5ccc(N(c6ccccc6)c6ccc(-c7cc8ccccc8c8ccccc78)cc6)cc5)cc4c3o2)cc1 ; c1ccc(-c2nc3c(ccc4ccc(-c5ccc(N(c6ccccc6)c6ccc(-c7cc8ccccc8c8ccccc78)cc6)cc5)cc43)o2)cc1 ; c1ccc(-c2cccc(N(c3ccc(-c4ccc5ccc6nc(-c7ccccc7)oc6c5c4)cc3)c3ccc(-c4cc5ccccc5c5ccccc45)cc3)c2)cc1]

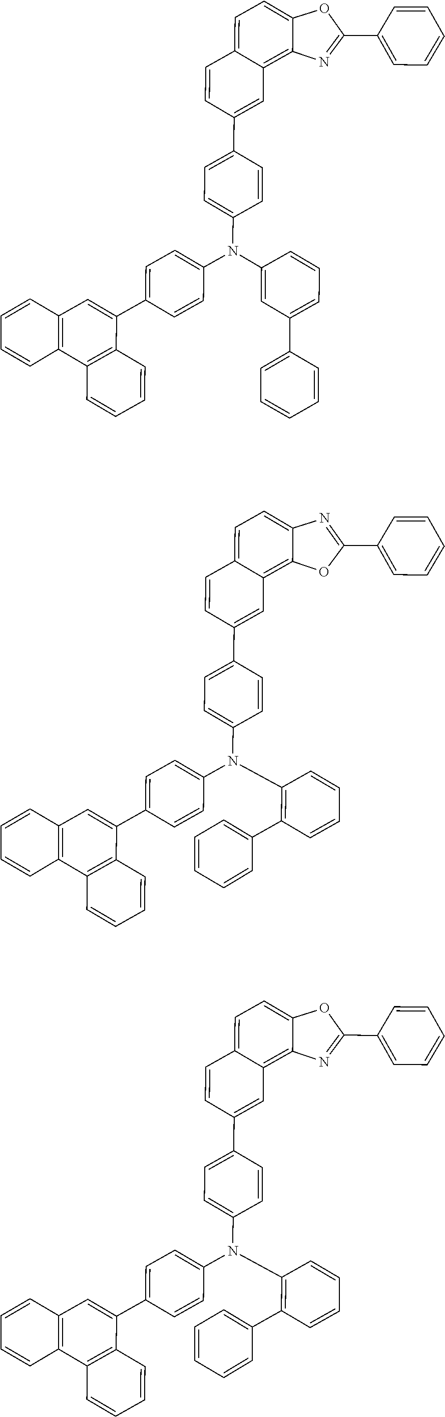 [SMILES: c1ccc(-c2cccc(N(c3ccc(-c4ccc5ccc6oc(-c7ccccc7)nc6c5c4)cc3)c3ccc(-c4cc5ccccc5c5ccccc45)cc3)c2)cc1 ; c1ccc(-c2nc3ccc4ccc(-c5ccc(N(c6ccc(-c7cc8ccccc8c8ccccc78)cc6)c6ccccc6-c6ccccc6)cc5)cc4c3o2)cc1 ; c1ccc(-c2nc3c(ccc4ccc(-c5ccc(N(c6ccc(-c7cc8ccccc8c8ccccc78)cc6)c6ccccc6-c6ccccc6)cc5)cc43)o2)cc1]

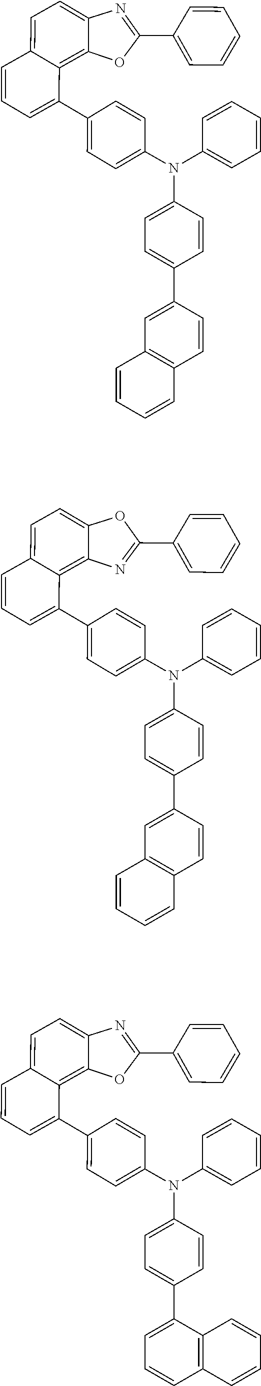 [SMILES: c1ccc(-c2nc3ccc4cccc(-c5ccc(N(c6ccccc6)c6ccc(-c7ccc8ccccc8c7)cc6)cc5)c4c3o2)cc1 ; c1ccc(-c2nc3c(ccc4cccc(-c5ccc(N(c6ccccc6)c6ccc(-c7ccc8ccccc8c7)cc6)cc5)c43)o2)cc1 ; c1ccc(-c2nc3ccc4cccc(-c5ccc(N(c6ccccc6)c6ccc(-c7cccc8ccccc78)cc6)cc5)c4c3o2)cc1]

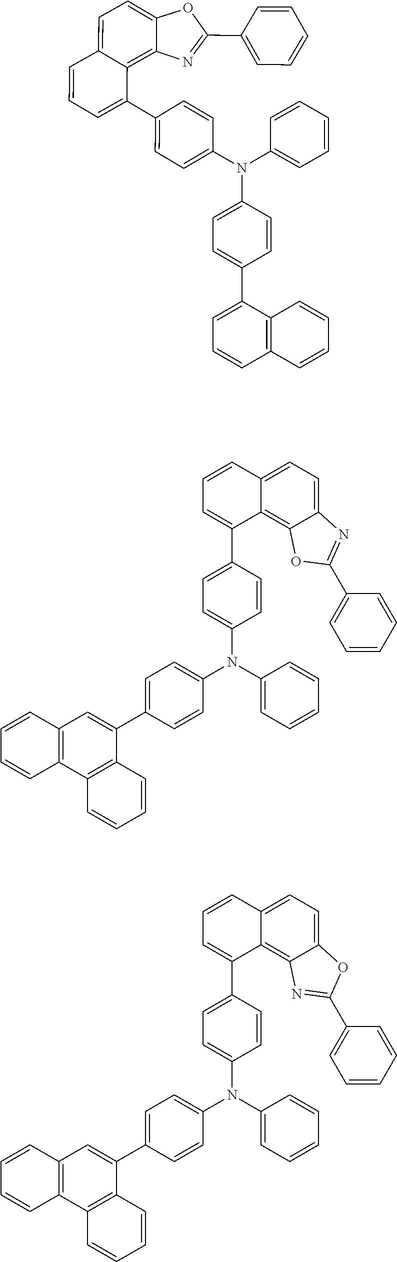 [SMILES: c1ccc(-c2nc3c(ccc4cccc(-c5ccc(N(c6ccccc6)c6ccc(-c7cccc8ccccc78)cc6)cc5)c43)o2)cc1 ; c1ccc(-c2nc3ccc4cccc(-c5ccc(N(c6ccccc6)c6ccc(-c7cc8ccccc8c8ccccc78)cc6)cc5)c4c3o2)cc1 ; c1ccc(-c2nc3c(ccc4cccc(-c5ccc(N(c6ccccc6)c6ccc(-c7cc8ccccc8c8ccccc78)cc6)cc5)c43)o2)cc1]

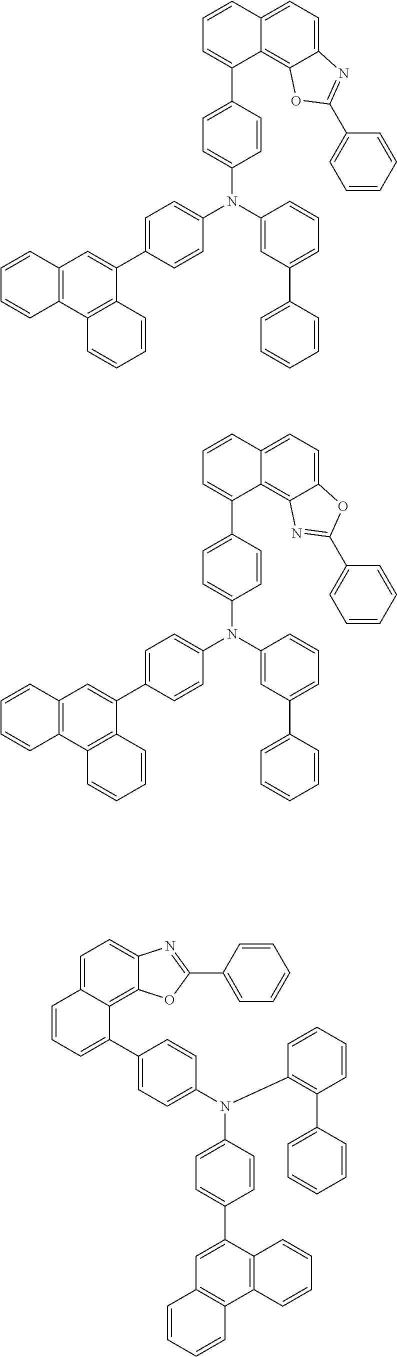 [SMILES: c1ccc(-c2cccc(N(c3ccc(-c4cc5ccccc5c5ccccc45)cc3)c3ccc(-c4cccc5ccc6nc(-c7ccccc7)oc6c45)cc3)c2)cc1 ; c1ccc(-c2cccc(N(c3ccc(-c4cc5ccccc5c5ccccc45)cc3)c3ccc(-c4cccc5ccc6oc(-c7ccccc7)nc6c45)cc3)c2)cc1 ; c1ccc(-c2nc3ccc4cccc(-c5ccc(N(c6ccc(-c7cc8ccccc8c8ccccc78)cc6)c6ccccc6-c6ccccc6)cc5)c4c3o2)cc1]

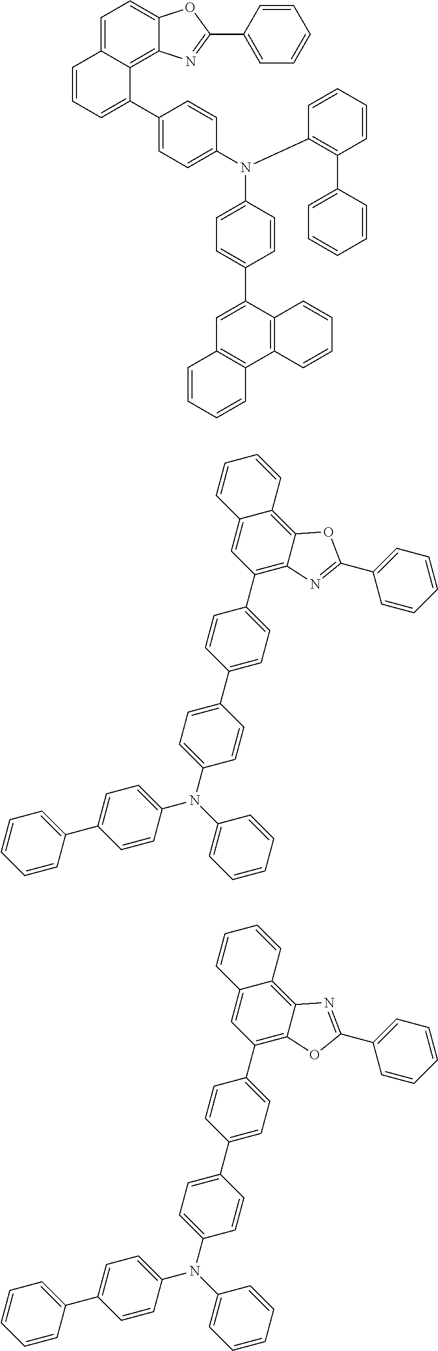

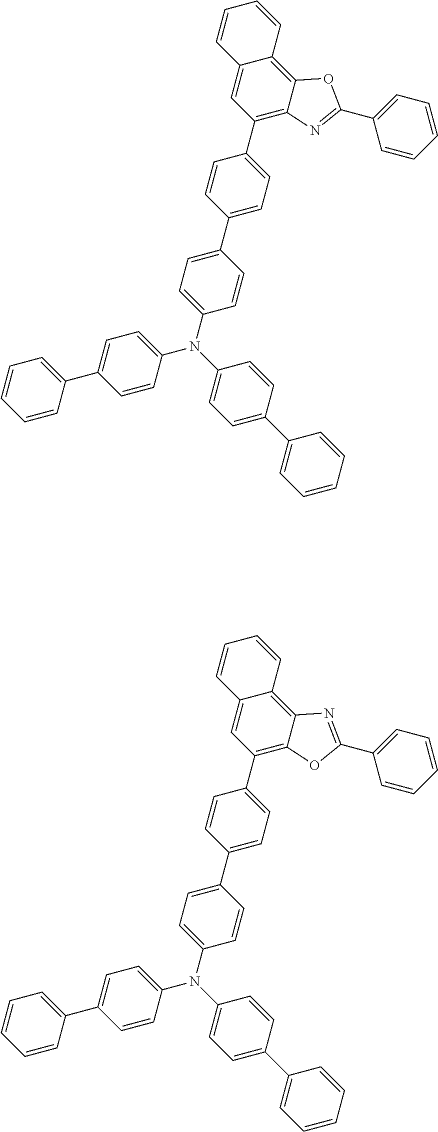 [SMILES: c1ccc(-c2ccc(N(c3ccc(-c4ccccc4)cc3)c3ccc(-c4ccc(-c5cc6ccccc6c6oc(-c7ccccc7)nc56)cc4)cc3)cc2)cc1 ; c1ccc(-c2ccc(N(c3ccc(-c4ccccc4)cc3)c3ccc(-c4ccc(-c5cc6ccccc6c6nc(-c7ccccc7)oc56)cc4)cc3)cc2)cc1]

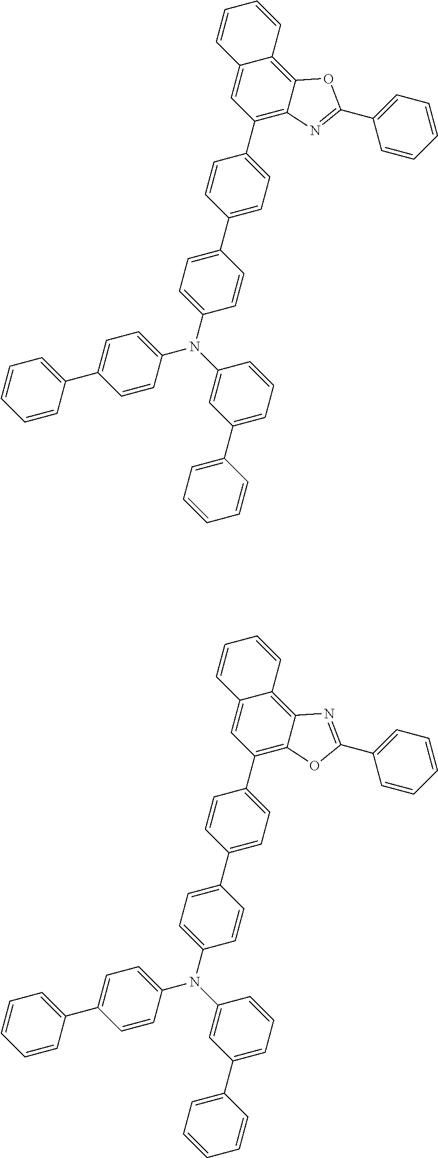 [SMILES: c1ccc(-c2ccc(N(c3ccc(-c4ccc(-c5cc6ccccc6c6oc(-c7ccccc7)nc56)cc4)cc3)c3cccc(-c4ccccc4)c3)cc2)cc1 ; c1ccc(-c2ccc(N(c3ccc(-c4ccc(-c5cc6ccccc6c6nc(-c7ccccc7)oc56)cc4)cc3)c3cccc(-c4ccccc4)c3)cc2)cc1]

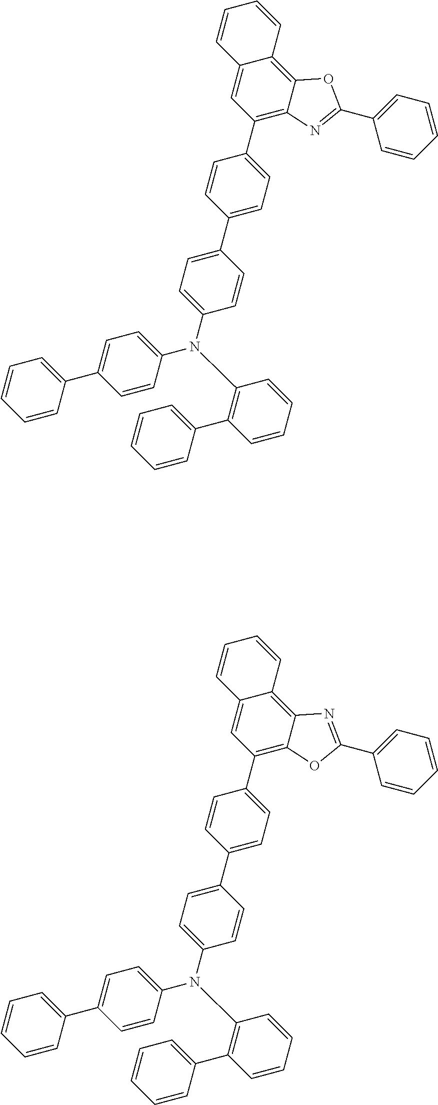 [SMILES: c1ccc(-c2ccc(N(c3ccc(-c4ccc(-c5cc6ccccc6c6oc(-c7ccccc7)nc56)cc4)cc3)c3ccccc3-c3ccccc3)cc2)cc1 ; c1ccc(-c2ccc(N(c3ccc(-c4ccc(-c5cc6ccccc6c6nc(-c7ccccc7)oc56)cc4)cc3)c3ccccc3-c3ccccc3)cc2)cc1]

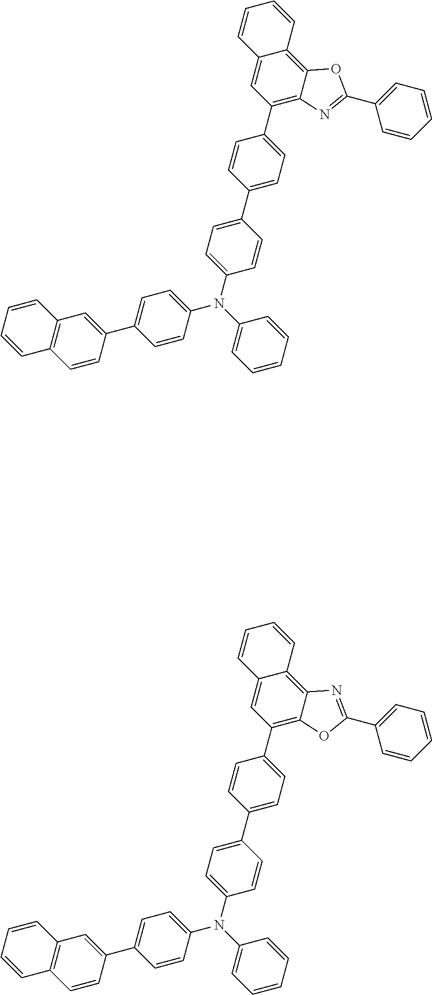 [SMILES: c1ccc(-c2nc3c(-c4ccc(-c5ccc(N(c6ccccc6)c6ccc(-c7ccc8ccccc8c7)cc6)cc5)cc4)cc4ccccc4c3o2)cc1 ; c1ccc(-c2nc3c(o2)c(-c2ccc(-c4ccc(N(c5ccccc5)c5ccc(-c6ccc7ccccc7c6)cc5)cc4)cc2)cc2ccccc23)cc1]

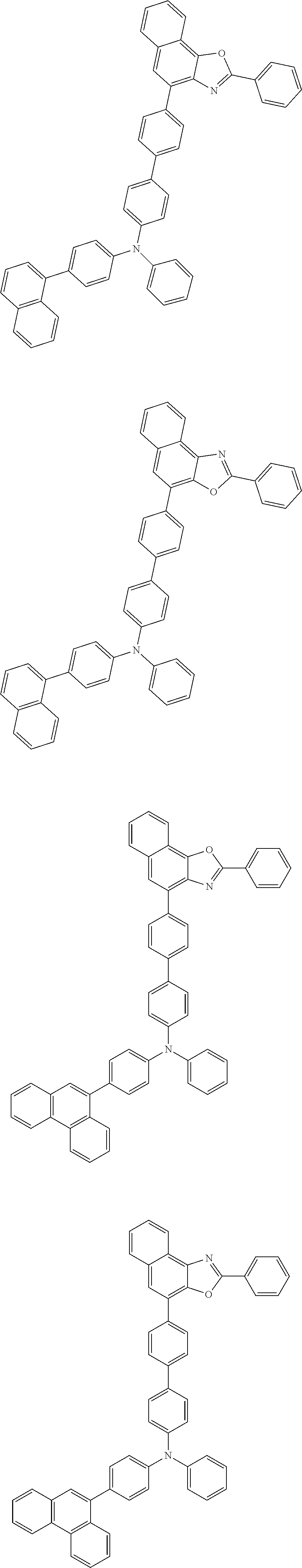 [SMILES: c1ccc(-c2nc3c(-c4ccc(-c5ccc(N(c6ccccc6)c6ccc(-c7cccc8ccccc78)cc6)cc5)cc4)cc4ccccc4c3o2)cc1 ; c1ccc(-c2nc3c(o2)c(-c2ccc(-c4ccc(N(c5ccccc5)c5ccc(-c6cccc7ccccc67)cc5)cc4)cc2)cc2ccccc23)cc1 ; c1ccc(-c2nc3c(-c4ccc(-c5ccc(N(c6ccccc6)c6ccc(-c7cc8ccccc8c8ccccc78)cc6)cc5)cc4)cc4ccccc4c3o2)cc1 ; c1ccc(-c2nc3c(o2)c(-c2ccc(-c4ccc(N(c5ccccc5)c5ccc(-c6cc7ccccc7c7ccccc67)cc5)cc4)cc2)cc2ccccc23)cc1]

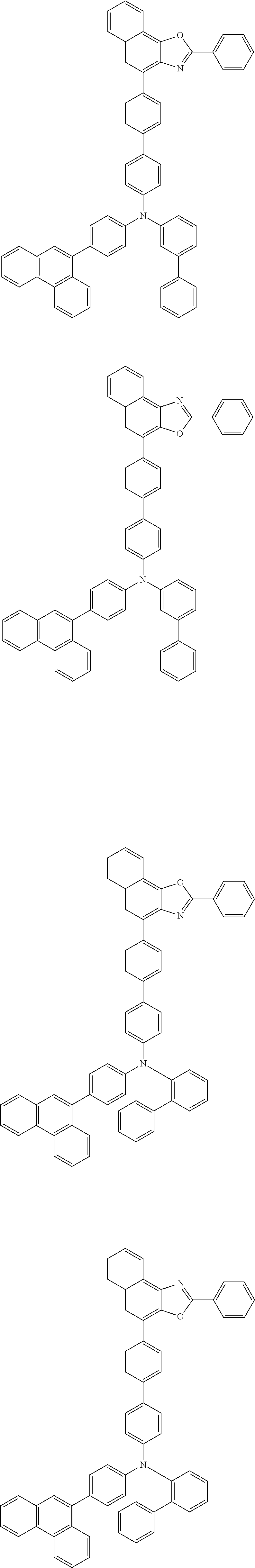 [SMILES: c1ccc(-c2cccc(N(c3ccc(-c4ccc(-c5cc6ccccc6c6oc(-c7ccccc7)nc56)cc4)cc3)c3ccc(-c4cc5ccccc5c5ccccc45)cc3)c2)cc1 ; c1ccc(-c2cccc(N(c3ccc(-c4ccc(-c5cc6ccccc6c6nc(-c7ccccc7)oc56)cc4)cc3)c3ccc(-c4cc5ccccc5c5ccccc45)cc3)c2)cc1 ; c1ccc(-c2nc3c(-c4ccc(-c5ccc(N(c6ccc(-c7cc8ccccc8c8ccccc78)cc6)c6ccccc6-c6ccccc6)cc5)cc4)cc4ccccc4c3o2)cc1 ; c1ccc(-c2nc3c(o2)c(-c2ccc(-c4ccc(N(c5ccc(-c6cc7ccccc7c7ccccc67)cc5)c5ccccc5-c5ccccc5)cc4)cc2)cc2ccccc23)cc1]

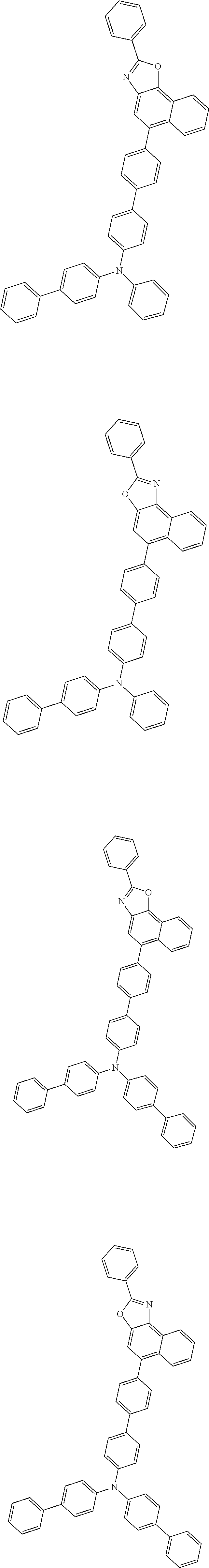 [SMILES: c1ccc(-c2ccc(N(c3ccccc3)c3ccc(-c4ccc(-c5cc6nc(-c7ccccc7)oc6c6ccccc56)cc4)cc3)cc2)cc1 ; c1ccc(-c2ccc(N(c3ccccc3)c3ccc(-c4ccc(-c5cc6oc(-c7ccccc7)nc6c6ccccc56)cc4)cc3)cc2)cc1 ; c1ccc(-c2ccc(N(c3ccc(-c4ccccc4)cc3)c3ccc(-c4ccc(-c5cc6nc(-c7ccccc7)oc6c6ccccc56)cc4)cc3)cc2)cc1 ; c1ccc(-c2ccc(N(c3ccc(-c4ccccc4)cc3)c3ccc(-c4ccc(-c5cc6oc(-c7ccccc7)nc6c6ccccc56)cc4)cc3)cc2)cc1]

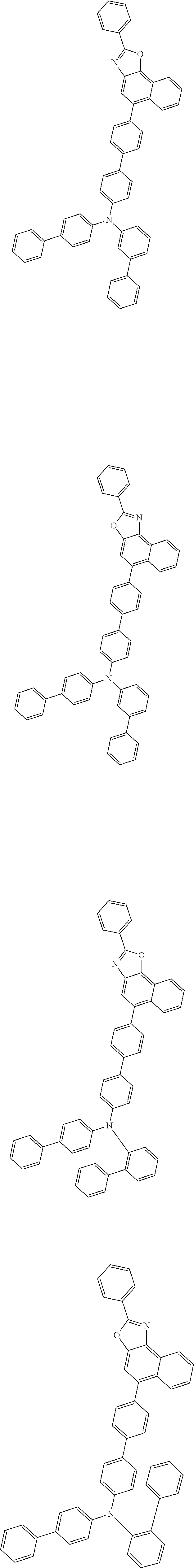 [SMILES: c1ccc(-c2ccc(N(c3ccc(-c4ccc(-c5cc6nc(-c7ccccc7)oc6c6ccccc56)cc4)cc3)c3cccc(-c4ccccc4)c3)cc2)cc1 ; c1ccc(-c2ccc(N(c3ccc(-c4ccc(-c5cc6oc(-c7ccccc7)nc6c6ccccc56)cc4)cc3)c3cccc(-c4ccccc4)c3)cc2)cc1 ; c1ccc(-c2ccc(N(c3ccc(-c4ccc(-c5cc6nc(-c7ccccc7)oc6c6ccccc56)cc4)cc3)c3ccccc3-c3ccccc3)cc2)cc1 ; c1ccc(-c2ccc(N(c3ccc(-c4ccc(-c5cc6oc(-c7ccccc7)nc6c6ccccc56)cc4)cc3)c3ccccc3-c3ccccc3)cc2)cc1]

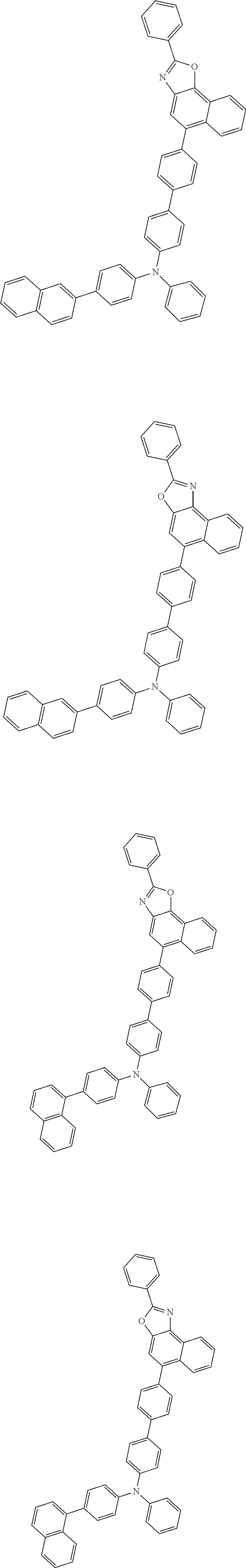 [SMILES: c1ccc(-c2nc3cc(-c4ccc(-c5ccc(N(c6ccccc6)c6ccc(-c7ccc8ccccc8c7)cc6)cc5)cc4)c4ccccc4c3o2)cc1 ; c1ccc(-c2nc3c(cc(-c4ccc(-c5ccc(N(c6ccccc6)c6ccc(-c7ccc8ccccc8c7)cc6)cc5)cc4)c4ccccc43)o2)cc1 ; c1ccc(-c2nc3cc(-c4ccc(-c5ccc(N(c6ccccc6)c6ccc(-c7cccc8ccccc78)cc6)cc5)cc4)c4ccccc4c3o2)cc1 ; c1ccc(-c2nc3c(cc(-c4ccc(-c5ccc(N(c6ccccc6)c6ccc(-c7cccc8ccccc78)cc6)cc5)cc4)c4ccccc43)o2)cc1]

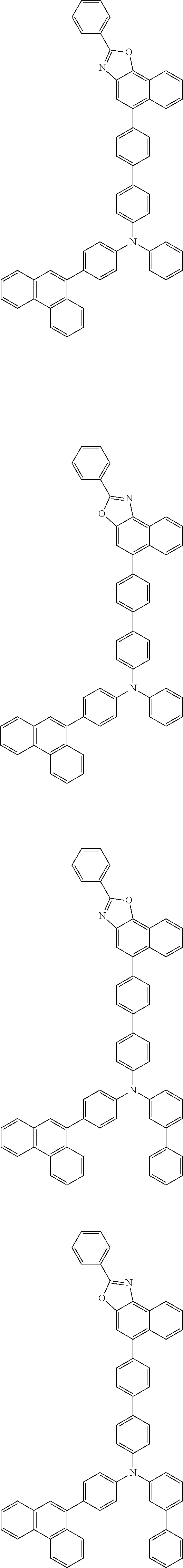 [SMILES: c1ccc(-c2nc3cc(-c4ccc(-c5ccc(N(c6ccccc6)c6ccc(-c7cc8ccccc8c8ccccc78)cc6)cc5)cc4)c4ccccc4c3o2)cc1 ; c1ccc(-c2nc3c(cc(-c4ccc(-c5ccc(N(c6ccccc6)c6ccc(-c7cc8ccccc8c8ccccc78)cc6)cc5)cc4)c4ccccc43)o2)cc1 ; c1ccc(-c2cccc(N(c3ccc(-c4ccc(-c5cc6nc(-c7ccccc7)oc6c6ccccc56)cc4)cc3)c3ccc(-c4cc5ccccc5c5ccccc45)cc3)c2)cc1 ; c1ccc(-c2cccc(N(c3ccc(-c4ccc(-c5cc6oc(-c7ccccc7)nc6c6ccccc56)cc4)cc3)c3ccc(-c4cc5ccccc5c5ccccc45)cc3)c2)cc1]

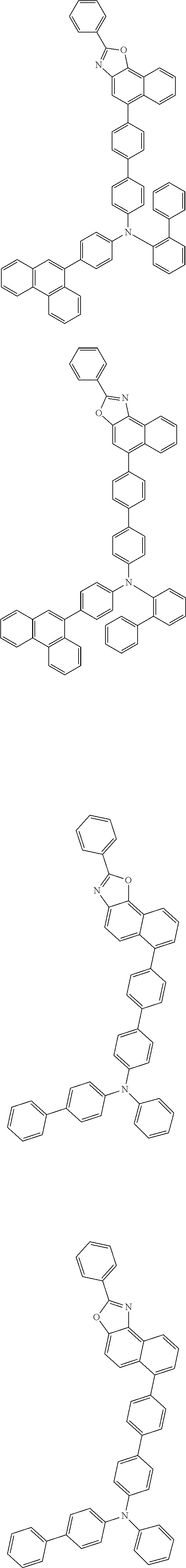 [SMILES: c1ccc(-c2nc3cc(-c4ccc(-c5ccc(N(c6ccc(-c7cc8ccccc8c8ccccc78)cc6)c6ccccc6-c6ccccc6)cc5)cc4)c4ccccc4c3o2)cc1 ; c1ccc(-c2nc3c(cc(-c4ccc(-c5ccc(N(c6ccc(-c7cc8ccccc8c8ccccc78)cc6)c6ccccc6-c6ccccc6)cc5)cc4)c4ccccc43)o2)cc1 ; c1ccc(-c2ccc(N(c3ccccc3)c3ccc(-c4ccc(-c5cccc6c5ccc5nc(-c7ccccc7)oc56)cc4)cc3)cc2)cc1 ; c1ccc(-c2ccc(N(c3ccccc3)c3ccc(-c4ccc(-c5cccc6c5ccc5oc(-c7ccccc7)nc56)cc4)cc3)cc2)cc1]

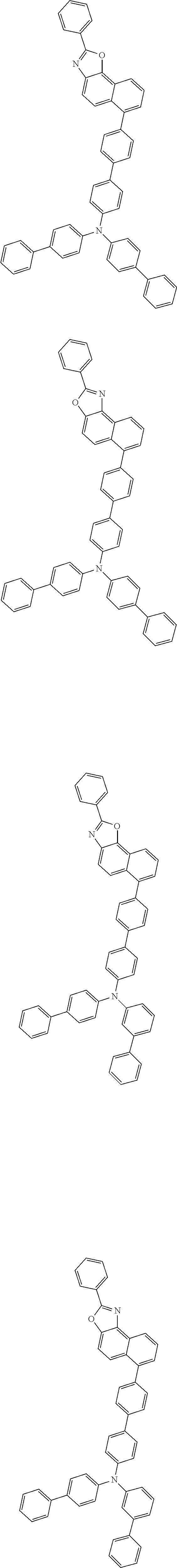 [SMILES: c1ccc(-c2ccc(N(c3ccc(-c4ccccc4)cc3)c3ccc(-c4ccc(-c5cccc6c5ccc5nc(-c7ccccc7)oc56)cc4)cc3)cc2)cc1 ; c1ccc(-c2ccc(N(c3ccc(-c4ccccc4)cc3)c3ccc(-c4ccc(-c5cccc6c5ccc5oc(-c7ccccc7)nc56)cc4)cc3)cc2)cc1 ; c1ccc(-c2ccc(N(c3ccc(-c4ccc(-c5cccc6c5ccc5nc(-c7ccccc7)oc56)cc4)cc3)c3cccc(-c4ccccc4)c3)cc2)cc1 ; c1ccc(-c2ccc(N(c3ccc(-c4ccc(-c5cccc6c5ccc5oc(-c7ccccc7)nc56)cc4)cc3)c3cccc(-c4ccccc4)c3)cc2)cc1]

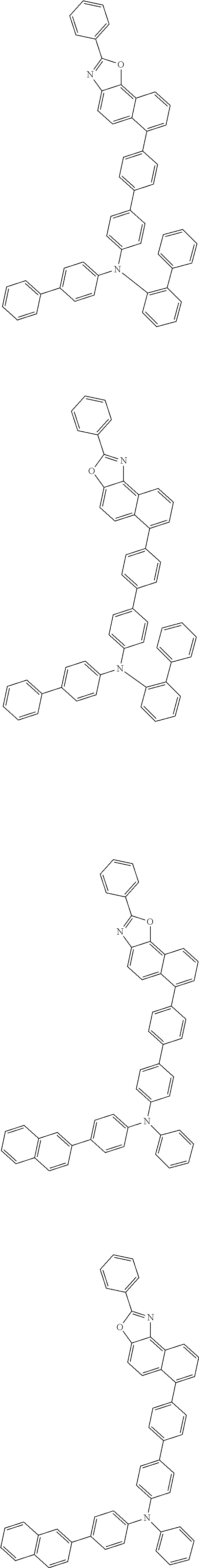 [SMILES: c1ccc(-c2ccc(N(c3ccc(-c4ccc(-c5cccc6c5ccc5nc(-c7ccccc7)oc56)cc4)cc3)c3ccccc3-c3ccccc3)cc2)cc1 ; c1ccc(-c2ccc(N(c3ccc(-c4ccc(-c5cccc6c5ccc5oc(-c7ccccc7)nc56)cc4)cc3)c3ccccc3-c3ccccc3)cc2)cc1 ; c1ccc(-c2nc3ccc4c(-c5ccc(-c6ccc(N(c7ccccc7)c7ccc(-c8ccc9ccccc9c8)cc7)cc6)cc5)cccc4c3o2)cc1 ; c1ccc(-c2nc3c(ccc4c(-c5ccc(-c6ccc(N(c7ccccc7)c7ccc(-c8ccc9ccccc9c8)cc7)cc6)cc5)cccc43)o2)cc1]

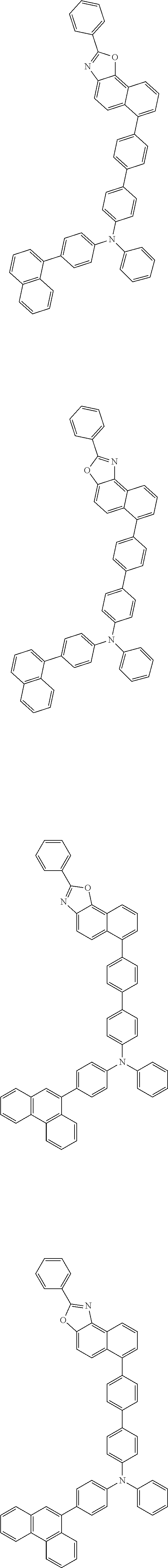 [SMILES: c1ccc(-c2nc3ccc4c(-c5ccc(-c6ccc(N(c7ccccc7)c7ccc(-c8cccc9ccccc89)cc7)cc6)cc5)cccc4c3o2)cc1 ; c1ccc(-c2nc3c(ccc4c(-c5ccc(-c6ccc(N(c7ccccc7)c7ccc(-c8cccc9ccccc89)cc7)cc6)cc5)cccc43)o2)cc1 ; c1ccc(-c2nc3ccc4c(-c5ccc(-c6ccc(N(c7ccccc7)c7ccc(-c8cc9ccccc9c9ccccc89)cc7)cc6)cc5)cccc4c3o2)cc1 ; c1ccc(-c2nc3c(ccc4c(-c5ccc(-c6ccc(N(c7ccccc7)c7ccc(-c8cc9ccccc9c9ccccc89)cc7)cc6)cc5)cccc43)o2)cc1]

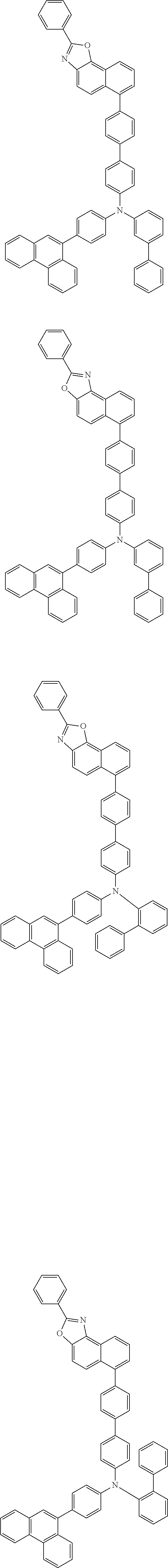 [SMILES: c1ccc(-c2cccc(N(c3ccc(-c4ccc(-c5cccc6c5ccc5nc(-c7ccccc7)oc56)cc4)cc3)c3ccc(-c4cc5ccccc5c5ccccc45)cc3)c2)cc1 ; c1ccc(-c2cccc(N(c3ccc(-c4ccc(-c5cccc6c5ccc5oc(-c7ccccc7)nc56)cc4)cc3)c3ccc(-c4cc5ccccc5c5ccccc45)cc3)c2)cc1 ; c1ccc(-c2nc3ccc4c(-c5ccc(-c6ccc(N(c7ccc(-c8cc9ccccc9c9ccccc89)cc7)c7ccccc7-c7ccccc7)cc6)cc5)cccc4c3o2)cc1 ; c1ccc(-c2nc3c(ccc4c(-c5ccc(-c6ccc(N(c7ccc(-c8cc9ccccc9c9ccccc89)cc7)c7ccccc7-c7ccccc7)cc6)cc5)cccc43)o2)cc1]

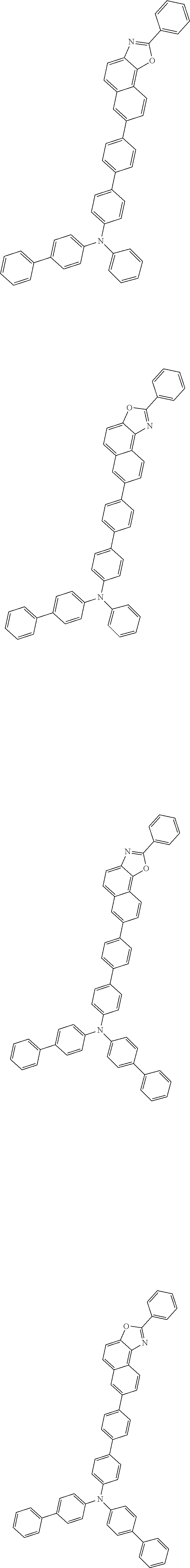 [SMILES: c1ccc(-c2ccc(N(c3ccccc3)c3ccc(-c4ccc(-c5ccc6c(ccc7nc(-c8ccccc8)oc76)c5)cc4)cc3)cc2)cc1 ; c1ccc(-c2ccc(N(c3ccccc3)c3ccc(-c4ccc(-c5ccc6c(ccc7oc(-c8ccccc8)nc76)c5)cc4)cc3)cc2)cc1 ; c1ccc(-c2ccc(N(c3ccc(-c4ccccc4)cc3)c3ccc(-c4ccc(-c5ccc6c(ccc7nc(-c8ccccc8)oc76)c5)cc4)cc3)cc2)cc1 ; c1ccc(-c2ccc(N(c3ccc(-c4ccccc4)cc3)c3ccc(-c4ccc(-c5ccc6c(ccc7oc(-c8ccccc8)nc76)c5)cc4)cc3)cc2)cc1]

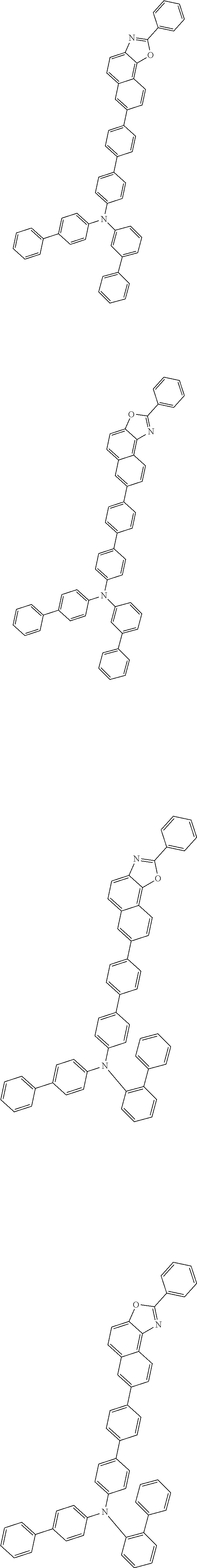 [SMILES: c1ccc(-c2ccc(N(c3ccc(-c4ccc(-c5ccc6c(ccc7nc(-c8ccccc8)oc76)c5)cc4)cc3)c3cccc(-c4ccccc4)c3)cc2)cc1 ; c1ccc(-c2ccc(N(c3ccc(-c4ccc(-c5ccc6c(ccc7oc(-c8ccccc8)nc76)c5)cc4)cc3)c3cccc(-c4ccccc4)c3)cc2)cc1 ; c1ccc(-c2ccc(N(c3ccc(-c4ccc(-c5ccc6c(ccc7nc(-c8ccccc8)oc76)c5)cc4)cc3)c3ccccc3-c3ccccc3)cc2)cc1 ; c1ccc(-c2ccc(N(c3ccc(-c4ccc(-c5ccc6c(ccc7oc(-c8ccccc8)nc76)c5)cc4)cc3)c3ccccc3-c3ccccc3)cc2)cc1]

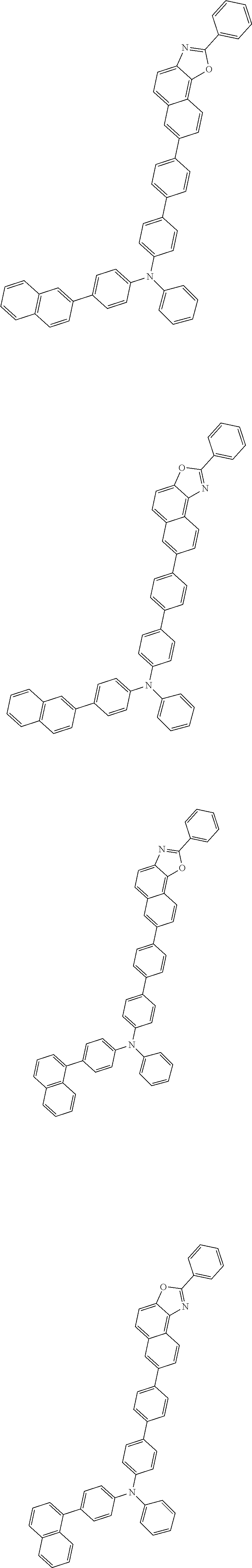 [SMILES: c1ccc(-c2nc3ccc4cc(-c5ccc(-c6ccc(N(c7ccccc7)c7ccc(-c8ccc9ccccc9c8)cc7)cc6)cc5)ccc4c3o2)cc1 ; c1ccc(-c2nc3c(ccc4cc(-c5ccc(-c6ccc(N(c7ccccc7)c7ccc(-c8ccc9ccccc9c8)cc7)cc6)cc5)ccc43)o2)cc1 ; c1ccc(-c2nc3ccc4cc(-c5ccc(-c6ccc(N(c7ccccc7)c7ccc(-c8cccc9ccccc89)cc7)cc6)cc5)ccc4c3o2)cc1 ; c1ccc(-c2nc3c(ccc4cc(-c5ccc(-c6ccc(N(c7ccccc7)c7ccc(-c8cccc9ccccc89)cc7)cc6)cc5)ccc43)o2)cc1]

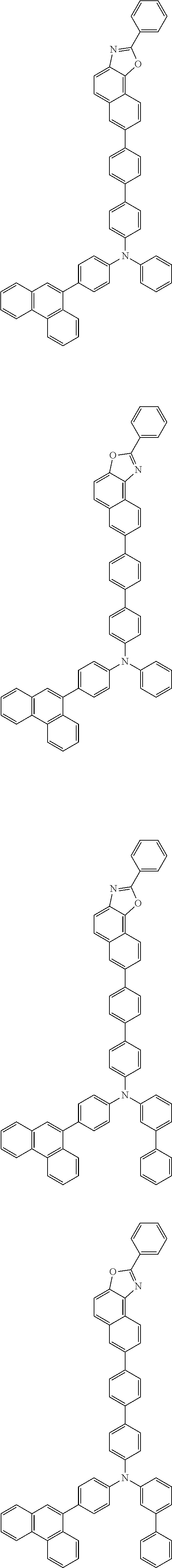 [SMILES: c1ccc(-c2nc3ccc4cc(-c5ccc(-c6ccc(N(c7ccccc7)c7ccc(-c8cc9ccccc9c9ccccc89)cc7)cc6)cc5)ccc4c3o2)cc1 ; c1ccc(-c2nc3c(ccc4cc(-c5ccc(-c6ccc(N(c7ccccc7)c7ccc(-c8cc9ccccc9c9ccccc89)cc7)cc6)cc5)ccc43)o2)cc1 ; c1ccc(-c2cccc(N(c3ccc(-c4ccc(-c5ccc6c(ccc7nc(-c8ccccc8)oc76)c5)cc4)cc3)c3ccc(-c4cc5ccccc5c5ccccc45)cc3)c2)cc1 ; c1ccc(-c2cccc(N(c3ccc(-c4ccc(-c5ccc6c(ccc7oc(-c8ccccc8)nc76)c5)cc4)cc3)c3ccc(-c4cc5ccccc5c5ccccc45)cc3)c2)cc1]

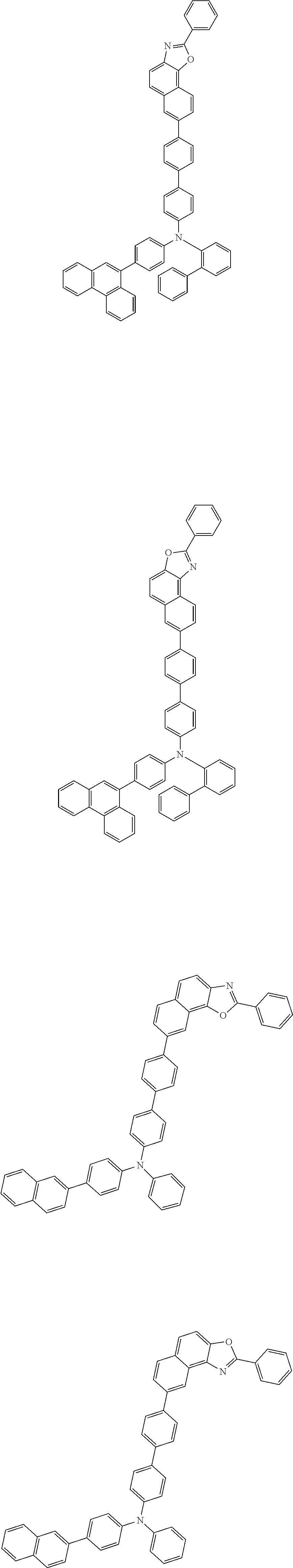 [SMILES: c1ccc(-c2nc3ccc4cc(-c5ccc(-c6ccc(N(c7ccc(-c8cc9ccccc9c9ccccc89)cc7)c7ccccc7-c7ccccc7)cc6)cc5)ccc4c3o2)cc1 ; c1ccc(-c2nc3c(ccc4cc(-c5ccc(-c6ccc(N(c7ccc(-c8cc9ccccc9c9ccccc89)cc7)c7ccccc7-c7ccccc7)cc6)cc5)ccc43)o2)cc1 ; c1ccc(-c2nc3ccc4ccc(-c5ccc(-c6ccc(N(c7ccccc7)c7ccc(-c8ccc9ccccc9c8)cc7)cc6)cc5)cc4c3o2)cc1 ; c1ccc(-c2nc3c(ccc4ccc(-c5ccc(-c6ccc(N(c7ccccc7)c7ccc(-c8ccc9ccccc9c8)cc7)cc6)cc5)cc43)o2)cc1]

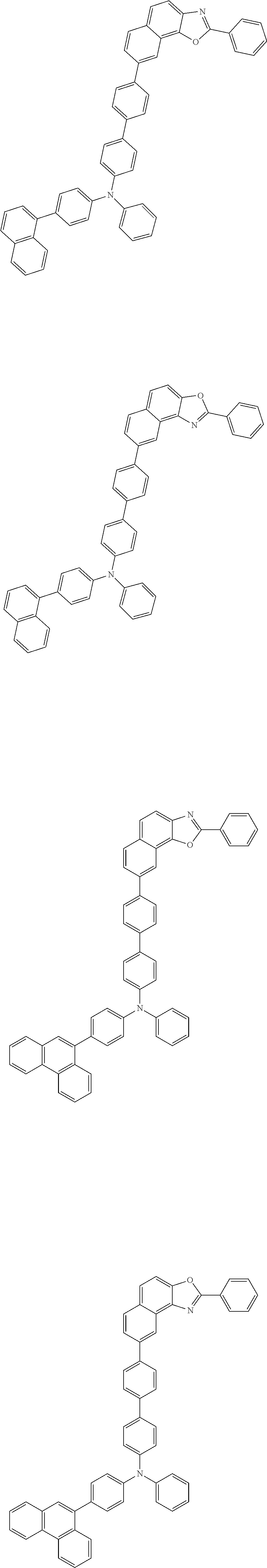 [SMILES: c1ccc(-c2nc3ccc4ccc(-c5ccc(-c6ccc(N(c7ccccc7)c7ccc(-c8cccc9ccccc89)cc7)cc6)cc5)cc4c3o2)cc1 ; c1ccc(-c2nc3c(ccc4ccc(-c5ccc(-c6ccc(N(c7ccccc7)c7ccc(-c8cccc9ccccc89)cc7)cc6)cc5)cc43)o2)cc1 ; c1ccc(-c2nc3ccc4ccc(-c5ccc(-c6ccc(N(c7ccccc7)c7ccc(-c8cc9ccccc9c9ccccc89)cc7)cc6)cc5)cc4c3o2)cc1 ; c1ccc(-c2nc3c(ccc4ccc(-c5ccc(-c6ccc(N(c7ccccc7)c7ccc(-c8cc9ccccc9c9ccccc89)cc7)cc6)cc5)cc43)o2)cc1]

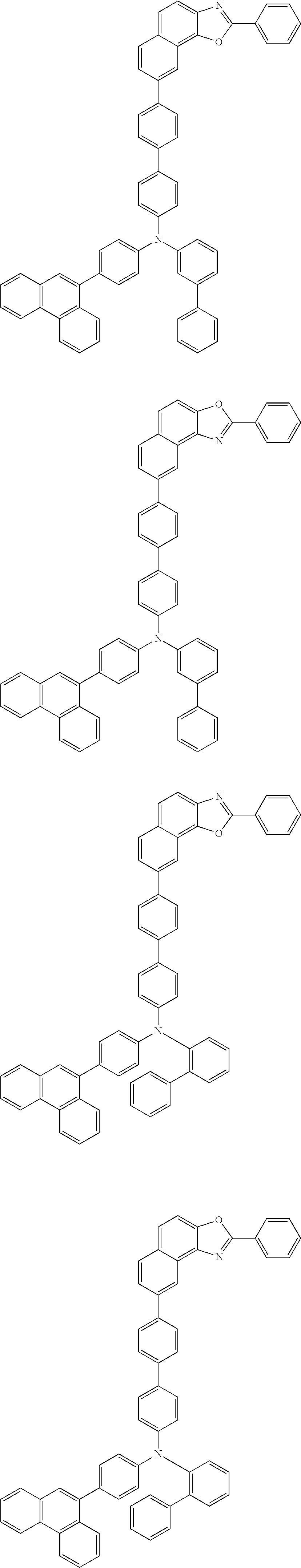 [SMILES: c1ccc(-c2cccc(N(c3ccc(-c4ccc(-c5ccc6ccc7nc(-c8ccccc8)oc7c6c5)cc4)cc3)c3ccc(-c4cc5ccccc5c5ccccc45)cc3)c2)cc1 ; c1ccc(-c2cccc(N(c3ccc(-c4ccc(-c5ccc6ccc7oc(-c8ccccc8)nc7c6c5)cc4)cc3)c3ccc(-c4cc5ccccc5c5ccccc45)cc3)c2)cc1 ; c1ccc(-c2nc3ccc4ccc(-c5ccc(-c6ccc(N(c7ccc(-c8cc9ccccc9c9ccccc89)cc7)c7ccccc7-c7ccccc7)cc6)cc5)cc4c3o2)cc1 ; c1ccc(-c2nc3c(ccc4ccc(-c5ccc(-c6ccc(N(c7ccc(-c8cc9ccccc9c9ccccc89)cc7)c7ccccc7-c7ccccc7)cc6)cc5)cc43)o2)cc1]

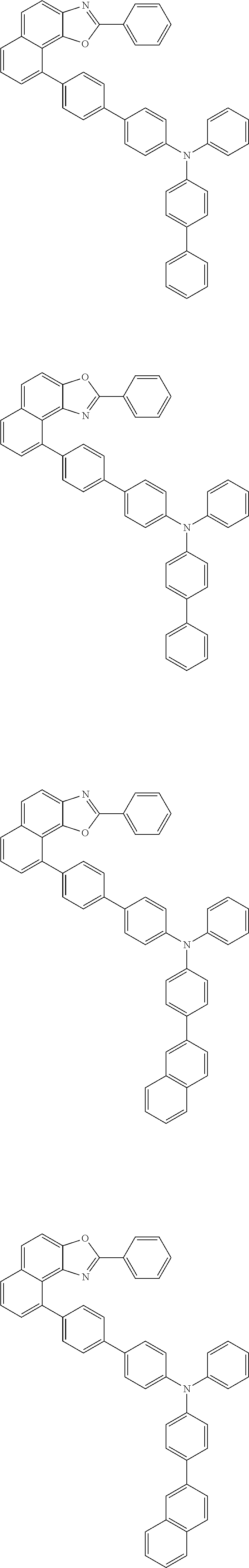 [SMILES: c1ccc(-c2ccc(N(c3ccccc3)c3ccc(-c4ccc(-c5cccc6ccc7nc(-c8ccccc8)oc7c56)cc4)cc3)cc2)cc1 ; c1ccc(-c2ccc(N(c3ccccc3)c3ccc(-c4ccc(-c5cccc6ccc7oc(-c8ccccc8)nc7c56)cc4)cc3)cc2)cc1 ; c1ccc(-c2nc3ccc4cccc(-c5ccc(-c6ccc(N(c7ccccc7)c7ccc(-c8ccc9ccccc9c8)cc7)cc6)cc5)c4c3o2)cc1 ; c1ccc(-c2nc3c(ccc4cccc(-c5ccc(-c6ccc(N(c7ccccc7)c7ccc(-c8ccc9ccccc9c8)cc7)cc6)cc5)c43)o2)cc1]

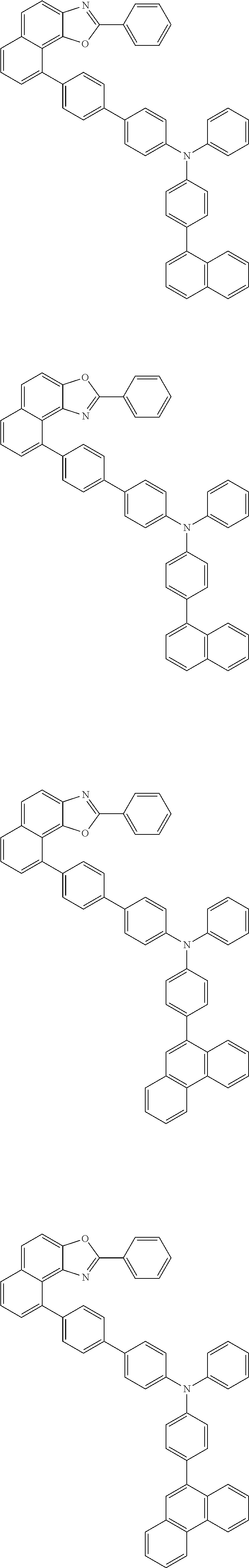 [SMILES: c1ccc(-c2nc3ccc4cccc(-c5ccc(-c6ccc(N(c7ccccc7)c7ccc(-c8cccc9ccccc89)cc7)cc6)cc5)c4c3o2)cc1 ; c1ccc(-c2nc3c(ccc4cccc(-c5ccc(-c6ccc(N(c7ccccc7)c7ccc(-c8cccc9ccccc89)cc7)cc6)cc5)c43)o2)cc1 ; c1ccc(-c2nc3ccc4cccc(-c5ccc(-c6ccc(N(c7ccccc7)c7ccc(-c8cc9ccccc9c9ccccc89)cc7)cc6)cc5)c4c3o2)cc1 ; c1ccc(-c2nc3c(ccc4cccc(-c5ccc(-c6ccc(N(c7ccccc7)c7ccc(-c8cc9ccccc9c9ccccc89)cc7)cc6)cc5)c43)o2)cc1]

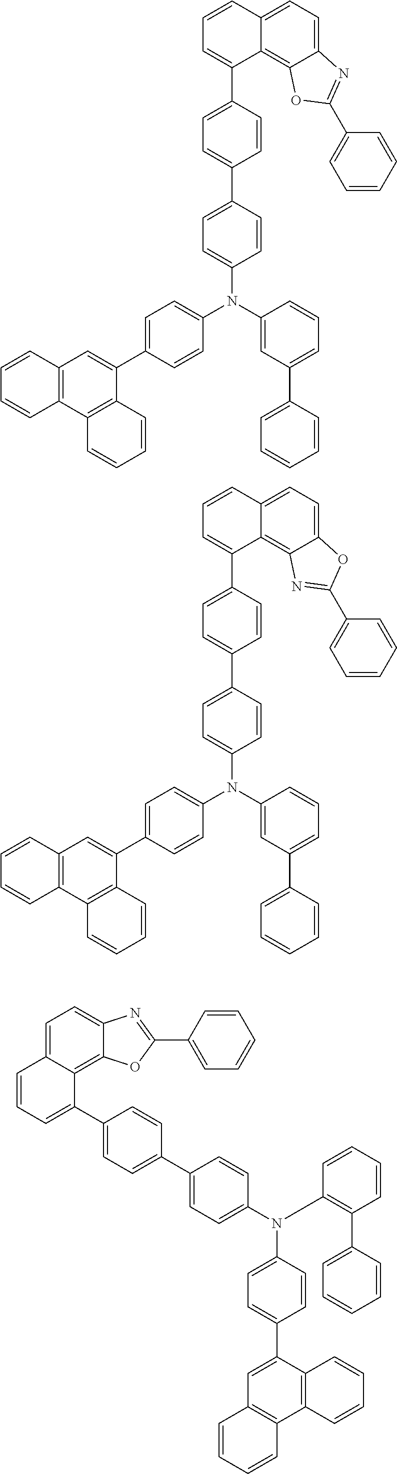

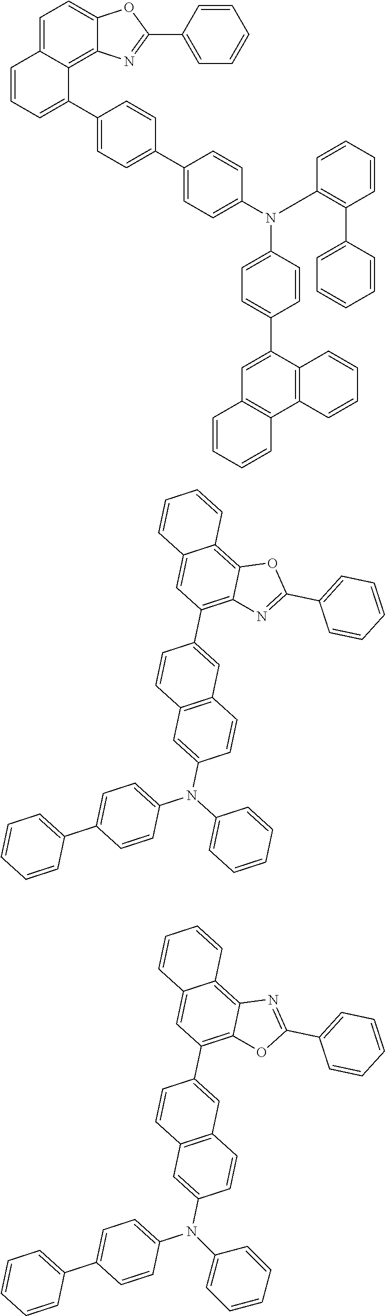

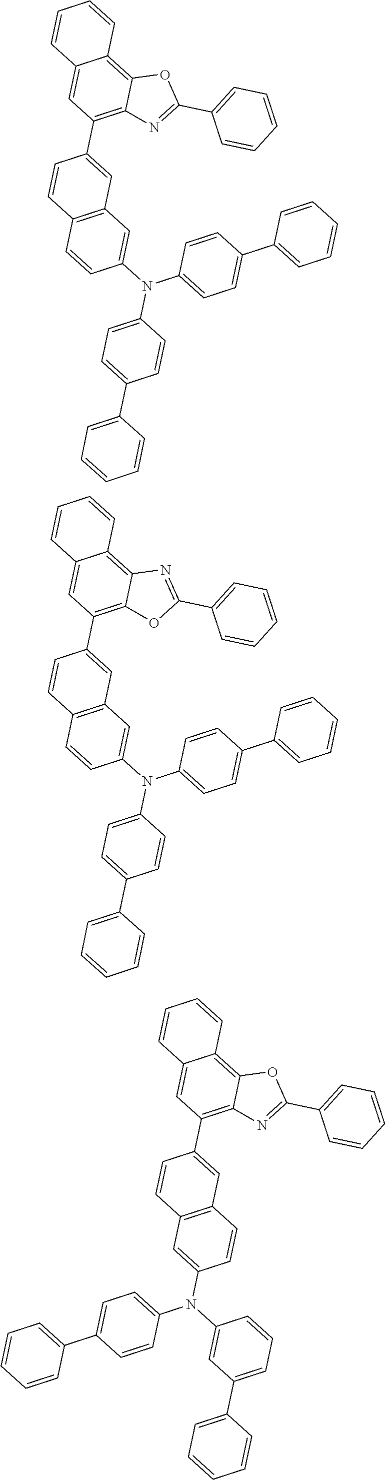

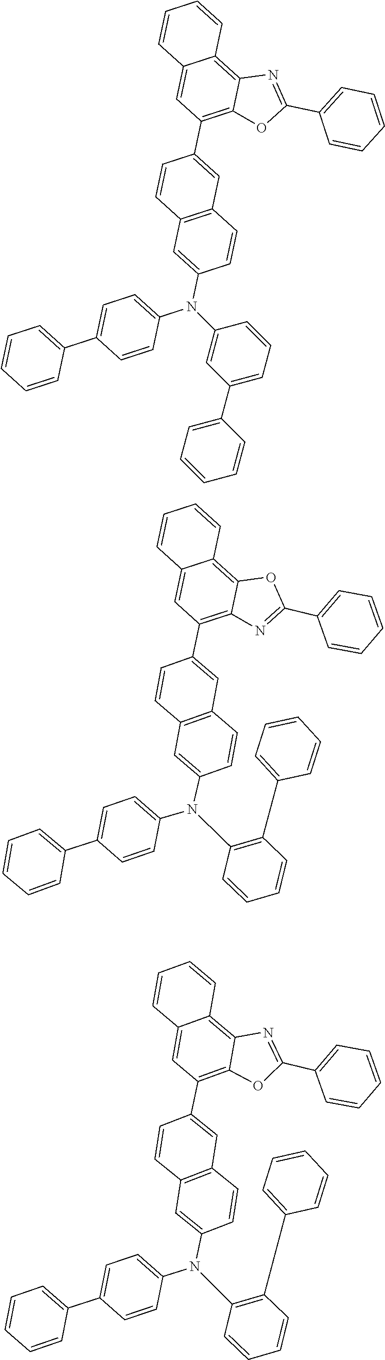

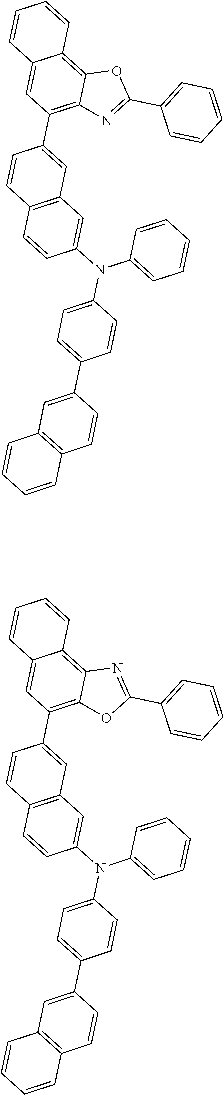 [SMILES: c1ccc(-c2nc3c(-c4ccc5ccc(N(c6ccccc6)c6ccc(-c7ccc8ccccc8c7)cc6)cc5c4)cc4ccccc4c3o2)cc1 ; c1ccc(-c2nc3c(o2)c(-c2ccc4ccc(N(c5ccccc5)c5ccc(-c6ccc7ccccc7c6)cc5)cc4c2)cc2ccccc23)cc1]

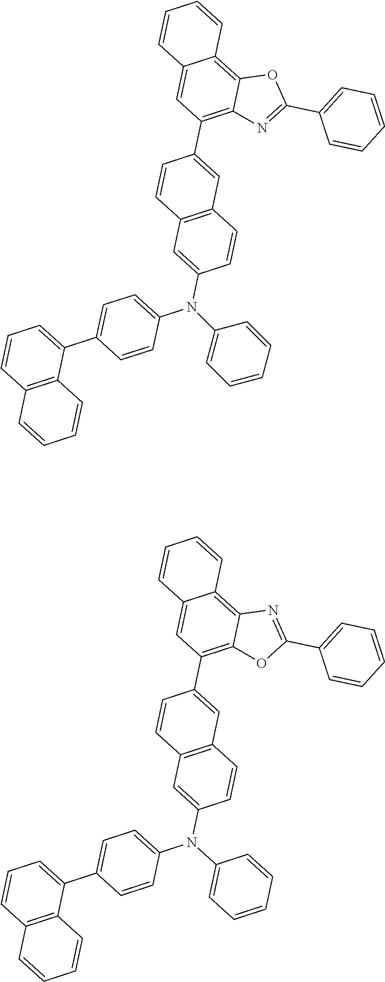 [SMILES: c1ccc(-c2nc3c(-c4ccc5cc(N(c6ccccc6)c6ccc(-c7cccc8ccccc78)cc6)ccc5c4)cc4ccccc4c3o2)cc1 ; c1ccc(-c2nc3c(o2)c(-c2ccc4cc(N(c5ccccc5)c5ccc(-c6cccc7ccccc67)cc5)ccc4c2)cc2ccccc23)cc1]

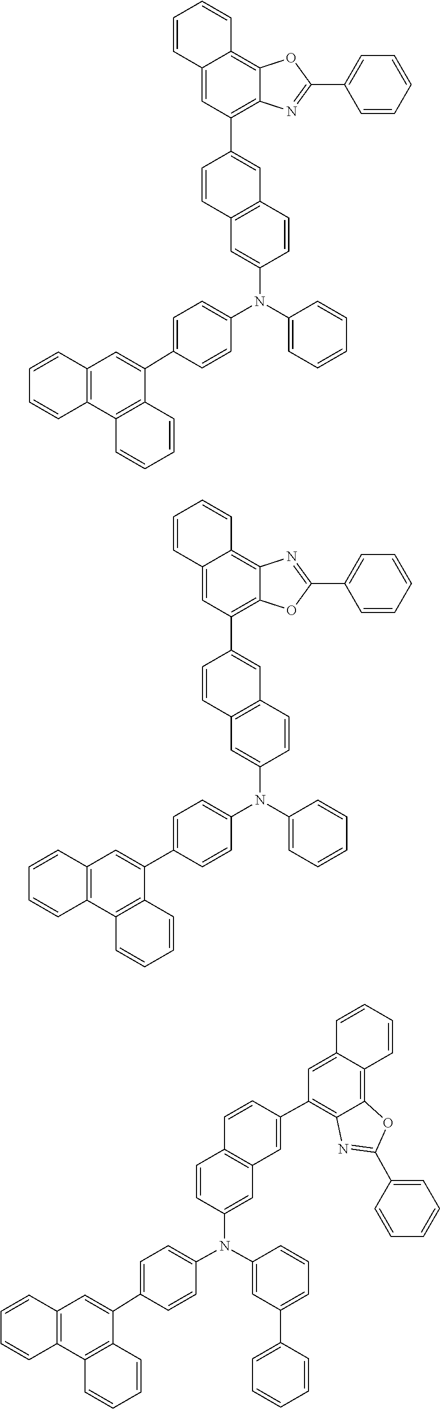 [SMILES: c1ccc(-c2nc3c(-c4ccc5cc(N(c6ccccc6)c6ccc(-c7cc8ccccc8c8ccccc78)cc6)ccc5c4)cc4ccccc4c3o2)cc1 ; c1ccc(-c2nc3c(o2)c(-c2ccc4cc(N(c5ccccc5)c5ccc(-c6cc7ccccc7c7ccccc67)cc5)ccc4c2)cc2ccccc23)cc1 ; c1ccc(-c2cccc(N(c3ccc(-c4cc5ccccc5c5ccccc45)cc3)c3ccc4ccc(-c5cc6ccccc6c6oc(-c7ccccc7)nc56)cc4c3)c2)cc1]

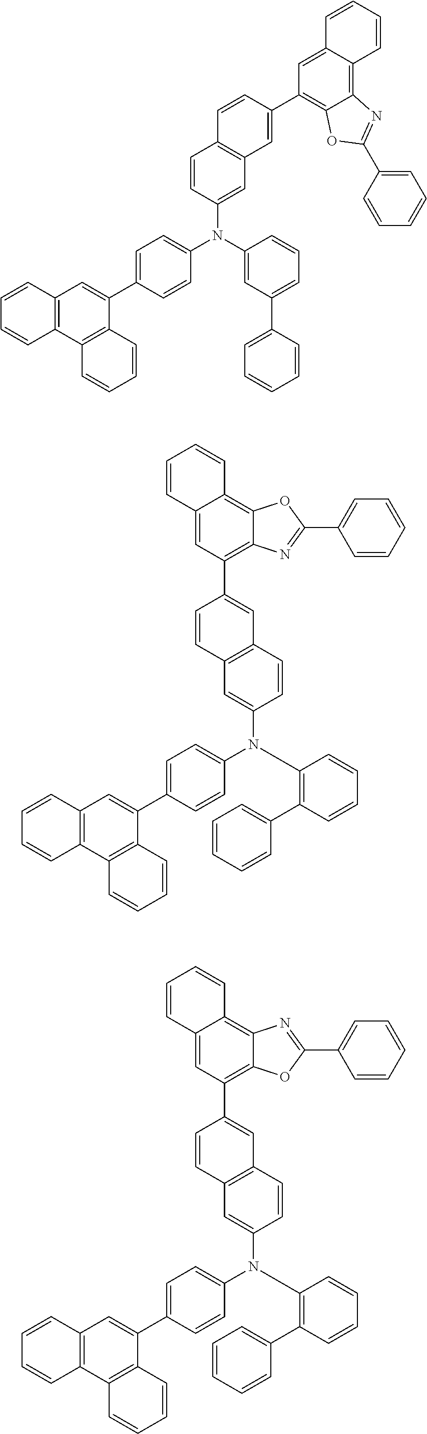 [SMILES: c1ccc(-c2cccc(N(c3ccc(-c4cc5ccccc5c5ccccc45)cc3)c3ccc4ccc(-c5cc6ccccc6c6nc(-c7ccccc7)oc56)cc4c3)c2)cc1 ; c1ccc(-c2nc3c(-c4ccc5cc(N(c6ccc(-c7cc8ccccc8c8ccccc78)cc6)c6ccccc6-c6ccccc6)ccc5c4)cc4ccccc4c3o2)cc1 ; c1ccc(-c2nc3c(o2)c(-c2ccc4cc(N(c5ccc(-c6cc7ccccc7c7ccccc67)cc5)c5ccccc5-c5ccccc5)ccc4c2)cc2ccccc23)cc1]

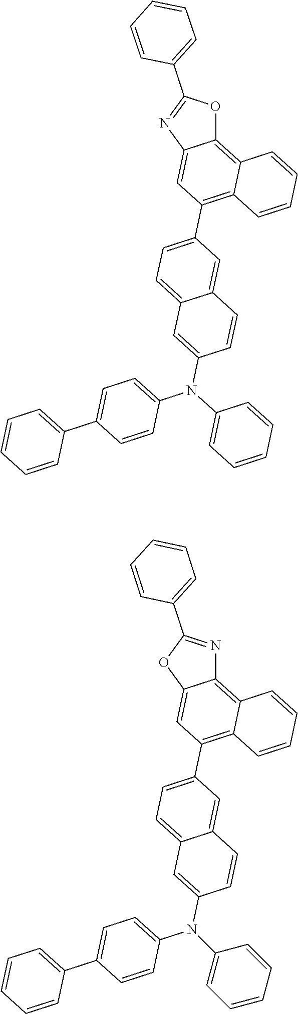 [SMILES: c1ccc(-c2ccc(N(c3ccccc3)c3ccc4cc(-c5cc6nc(-c7ccccc7)oc6c6ccccc56)ccc4c3)cc2)cc1 ; c1ccc(-c2ccc(N(c3ccccc3)c3ccc4cc(-c5cc6oc(-c7ccccc7)nc6c6ccccc56)ccc4c3)cc2)cc1]

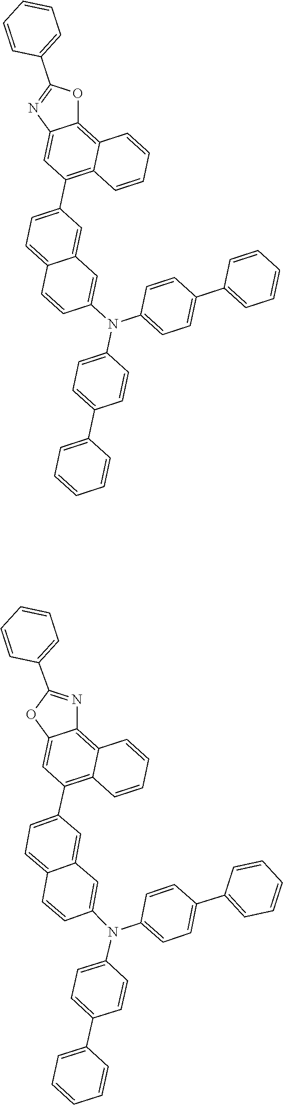 [SMILES: c1ccc(-c2ccc(N(c3ccc(-c4ccccc4)cc3)c3ccc4ccc(-c5cc6nc(-c7ccccc7)oc6c6ccccc56)cc4c3)cc2)cc1 ; c1ccc(-c2ccc(N(c3ccc(-c4ccccc4)cc3)c3ccc4ccc(-c5cc6oc(-c7ccccc7)nc6c6ccccc56)cc4c3)cc2)cc1]

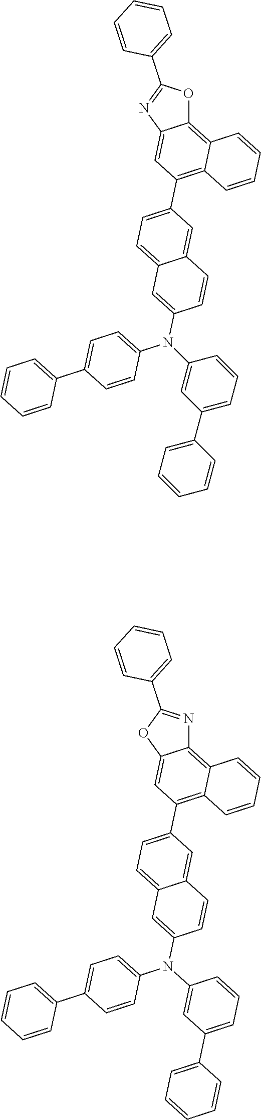 [SMILES: c1ccc(-c2ccc(N(c3cccc(-c4ccccc4)c3)c3ccc4cc(-c5cc6nc(-c7ccccc7)oc6c6ccccc56)ccc4c3)cc2)cc1 ; c1ccc(-c2ccc(N(c3cccc(-c4ccccc4)c3)c3ccc4cc(-c5cc6oc(-c7ccccc7)nc6c6ccccc56)ccc4c3)cc2)cc1]

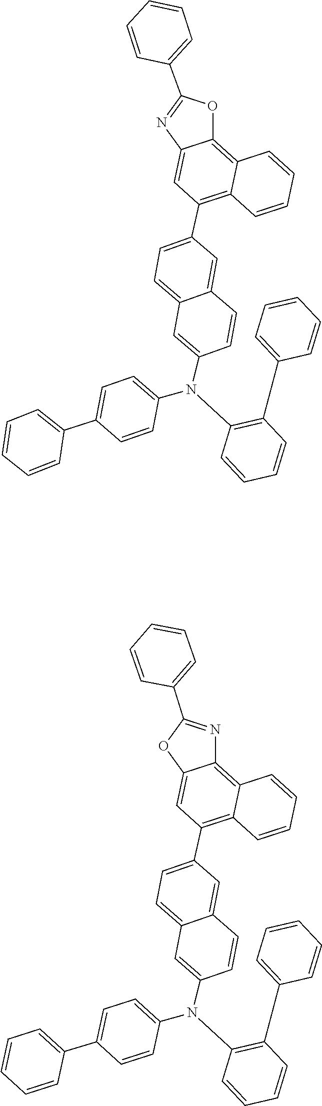 [SMILES: c1ccc(-c2ccc(N(c3ccc4cc(-c5cc6nc(-c7ccccc7)oc6c6ccccc56)ccc4c3)c3ccccc3-c3ccccc3)cc2)cc1 ; c1ccc(-c2ccc(N(c3ccc4cc(-c5cc6oc(-c7ccccc7)nc6c6ccccc56)ccc4c3)c3ccccc3-c3ccccc3)cc2)cc1]

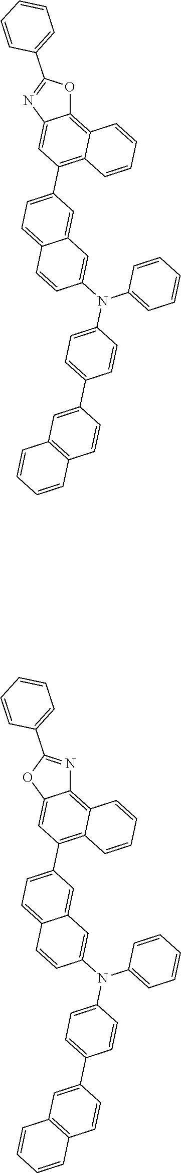 [SMILES: c1ccc(-c2nc3cc(-c4ccc5ccc(N(c6ccccc6)c6ccc(-c7ccc8ccccc8c7)cc6)cc5c4)c4ccccc4c3o2)cc1 ; c1ccc(-c2nc3c(cc(-c4ccc5ccc(N(c6ccccc6)c6ccc(-c7ccc8ccccc8c7)cc6)cc5c4)c4ccccc43)o2)cc1]

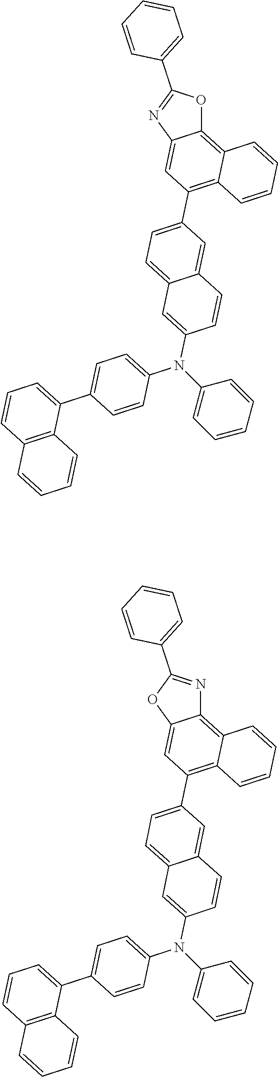 [SMILES: c1ccc(-c2nc3cc(-c4ccc5cc(N(c6ccccc6)c6ccc(-c7cccc8ccccc78)cc6)ccc5c4)c4ccccc4c3o2)cc1 ; c1ccc(-c2nc3c(cc(-c4ccc5cc(N(c6ccccc6)c6ccc(-c7cccc8ccccc78)cc6)ccc5c4)c4ccccc43)o2)cc1]

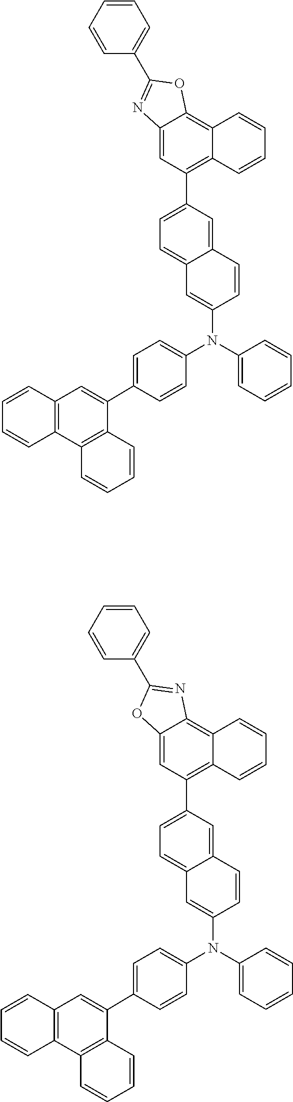 [SMILES: c1ccc(-c2nc3cc(-c4ccc5cc(N(c6ccccc6)c6ccc(-c7cc8ccccc8c8ccccc78)cc6)ccc5c4)c4ccccc4c3o2)cc1 ; c1ccc(-c2nc3c(cc(-c4ccc5cc(N(c6ccccc6)c6ccc(-c7cc8ccccc8c8ccccc78)cc6)ccc5c4)c4ccccc43)o2)cc1]

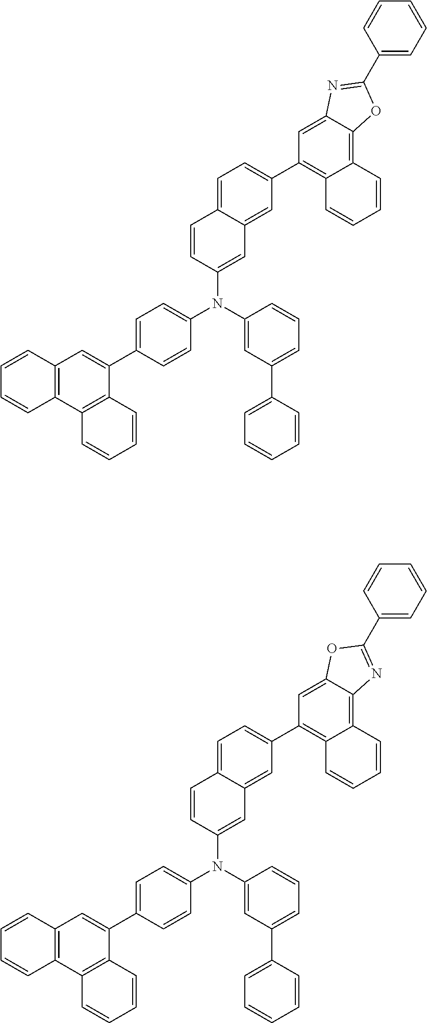 [SMILES: c1ccc(-c2cccc(N(c3ccc(-c4cc5ccccc5c5ccccc45)cc3)c3ccc4ccc(-c5cc6nc(-c7ccccc7)oc6c6ccccc56)cc4c3)c2)cc1 ; c1ccc(-c2cccc(N(c3ccc(-c4cc5ccccc5c5ccccc45)cc3)c3ccc4ccc(-c5cc6oc(-c7ccccc7)nc6c6ccccc56)cc4c3)c2)cc1]

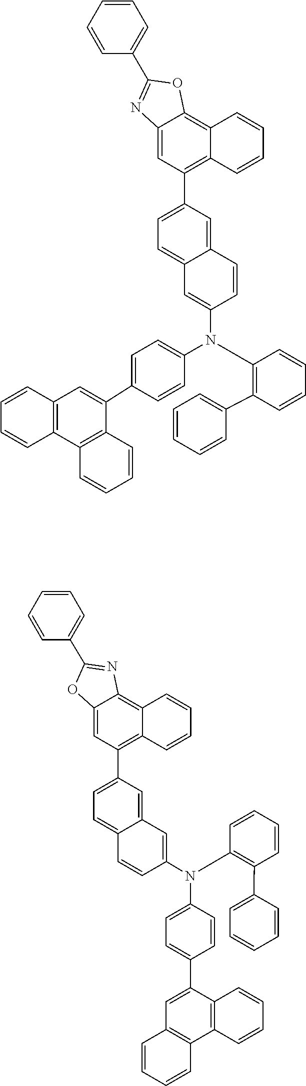 [SMILES: c1ccc(-c2nc3cc(-c4ccc5cc(N(c6ccc(-c7cc8ccccc8c8ccccc78)cc6)c6ccccc6-c6ccccc6)ccc5c4)c4ccccc4c3o2)cc1 ; c1ccc(-c2nc3c(cc(-c4ccc5ccc(N(c6ccc(-c7cc8ccccc8c8ccccc78)cc6)c6ccccc6-c6ccccc6)cc5c4)c4ccccc43)o2)cc1]

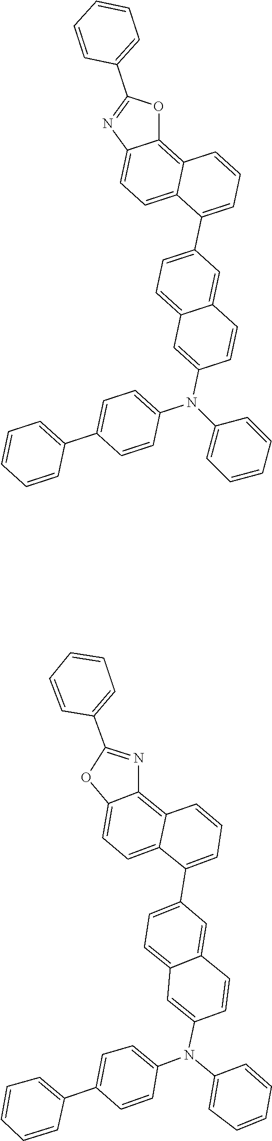

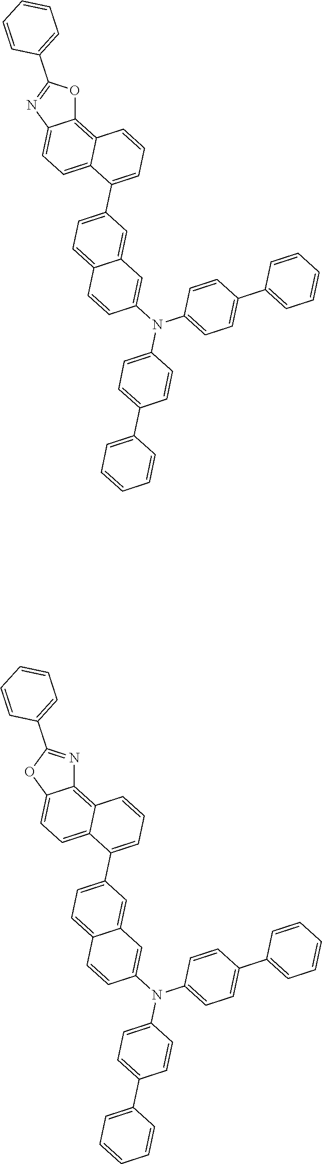 [SMILES: c1ccc(-c2ccc(N(c3ccc(-c4ccccc4)cc3)c3ccc4ccc(-c5cccc6c5ccc5nc(-c7ccccc7)oc56)cc4c3)cc2)cc1 ; c1ccc(-c2ccc(N(c3ccc(-c4ccccc4)cc3)c3ccc4ccc(-c5cccc6c5ccc5oc(-c7ccccc7)nc56)cc4c3)cc2)cc1]

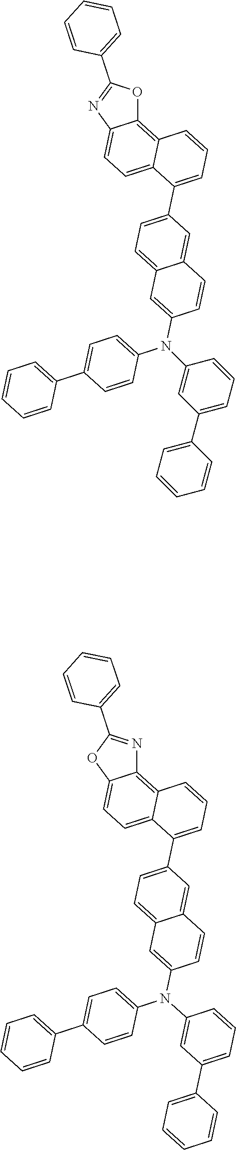

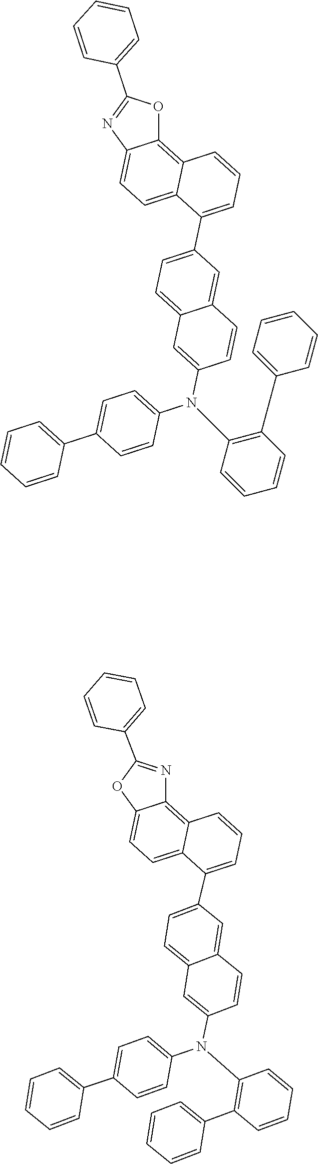 [SMILES: c1ccc(-c2ccc(N(c3ccc4cc(-c5cccc6c5ccc5nc(-c7ccccc7)oc56)ccc4c3)c3ccccc3-c3ccccc3)cc2)cc1 ; c1ccc(-c2ccc(N(c3ccc4cc(-c5cccc6c5ccc5oc(-c7ccccc7)nc56)ccc4c3)c3ccccc3-c3ccccc3)cc2)cc1]

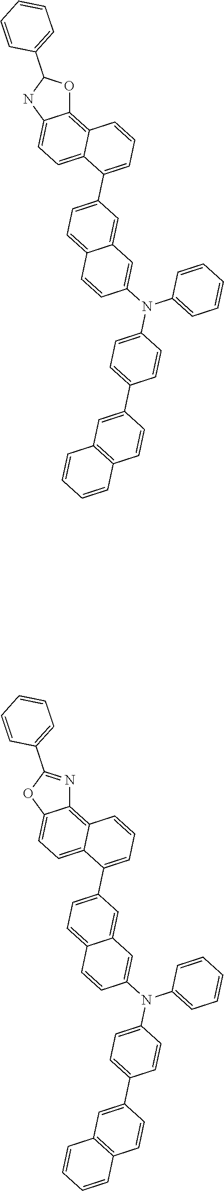

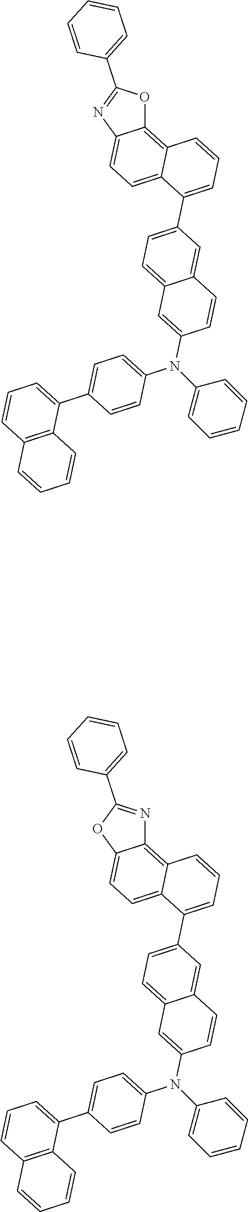 [SMILES: c1ccc(-c2nc3ccc4c(-c5ccc6cc(N(c7ccccc7)c7ccc(-c8cccc9ccccc89)cc7)ccc6c5)cccc4c3o2)cc1 ; c1ccc(-c2nc3c(ccc4c(-c5ccc6cc(N(c7ccccc7)c7ccc(-c8cccc9ccccc89)cc7)ccc6c5)cccc43)o2)cc1]

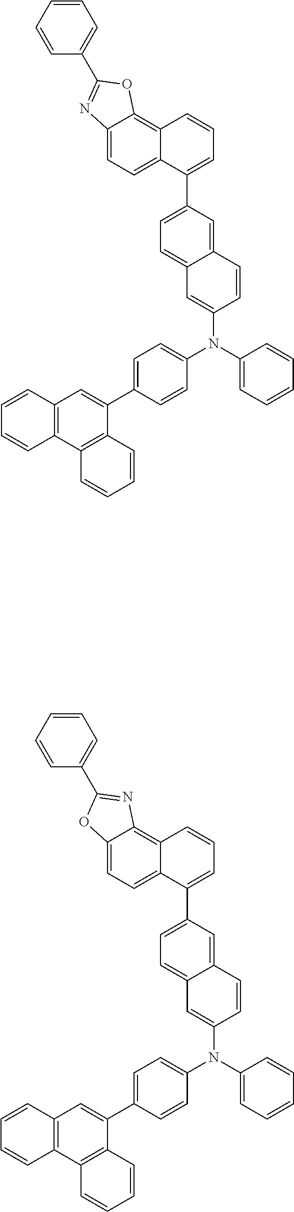 [SMILES: c1ccc(-c2nc3ccc4c(-c5ccc6cc(N(c7ccccc7)c7ccc(-c8cc9ccccc9c9ccccc89)cc7)ccc6c5)cccc4c3o2)cc1 ; c1ccc(-c2nc3c(ccc4c(-c5ccc6cc(N(c7ccccc7)c7ccc(-c8cc9ccccc9c9ccccc89)cc7)ccc6c5)cccc43)o2)cc1]

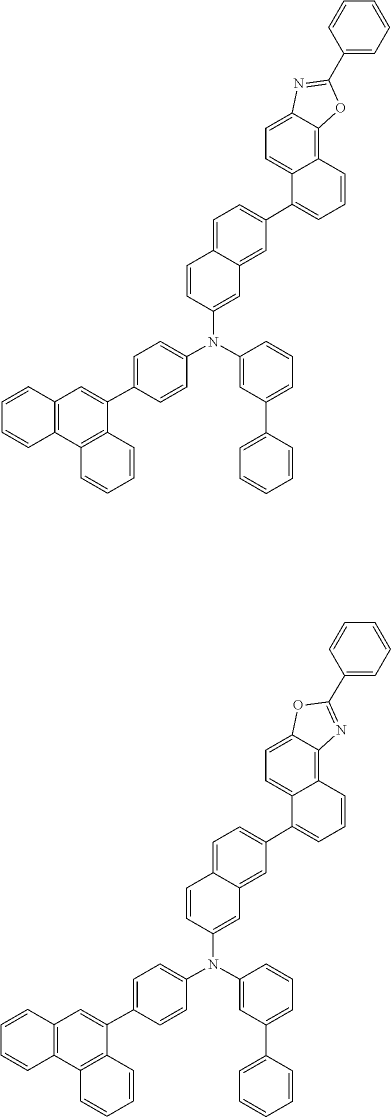 [SMILES: c1ccc(-c2cccc(N(c3ccc(-c4cc5ccccc5c5ccccc45)cc3)c3ccc4ccc(-c5cccc6c5ccc5nc(-c7ccccc7)oc56)cc4c3)c2)cc1 ; c1ccc(-c2cccc(N(c3ccc(-c4cc5ccccc5c5ccccc45)cc3)c3ccc4ccc(-c5cccc6c5ccc5oc(-c7ccccc7)nc56)cc4c3)c2)cc1]

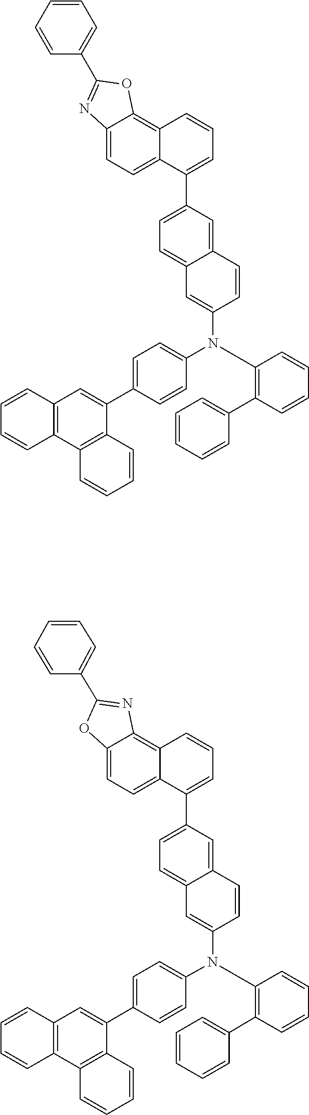 [SMILES: c1ccc(-c2nc3ccc4c(-c5ccc6cc(N(c7ccc(-c8cc9ccccc9c9ccccc89)cc7)c7ccccc7-c7ccccc7)ccc6c5)cccc4c3o2)cc1 ; c1ccc(-c2nc3c(ccc4c(-c5ccc6cc(N(c7ccc(-c8cc9ccccc9c9ccccc89)cc7)c7ccccc7-c7ccccc7)ccc6c5)cccc43)o2)cc1]

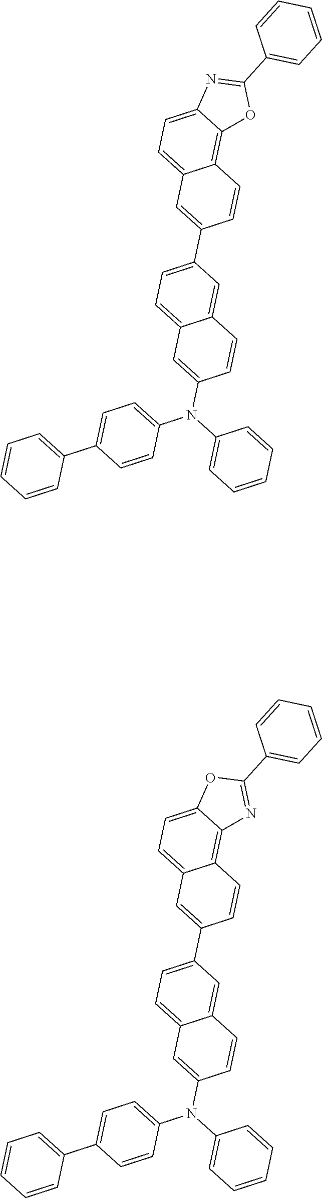 [SMILES: c1ccc(-c2ccc(N(c3ccccc3)c3ccc4cc(-c5ccc6c(ccc7nc(-c8ccccc8)oc76)c5)ccc4c3)cc2)cc1 ; c1ccc(-c2ccc(N(c3ccccc3)c3ccc4cc(-c5ccc6c(ccc7oc(-c8ccccc8)nc76)c5)ccc4c3)cc2)cc1]

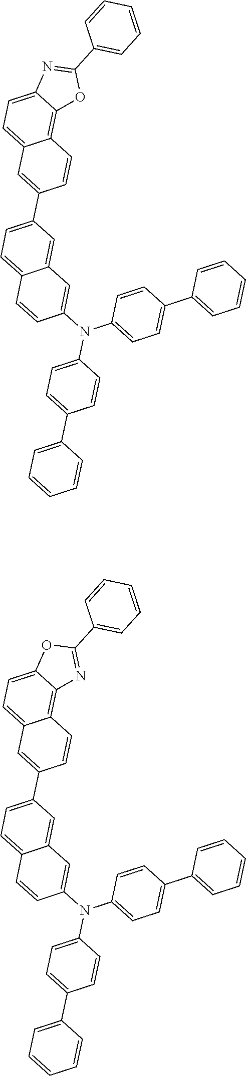 [SMILES: c1ccc(-c2ccc(N(c3ccc(-c4ccccc4)cc3)c3ccc4ccc(-c5ccc6c(ccc7nc(-c8ccccc8)oc76)c5)cc4c3)cc2)cc1 ; c1ccc(-c2ccc(N(c3ccc(-c4ccccc4)cc3)c3ccc4ccc(-c5ccc6c(ccc7oc(-c8ccccc8)nc76)c5)cc4c3)cc2)cc1]

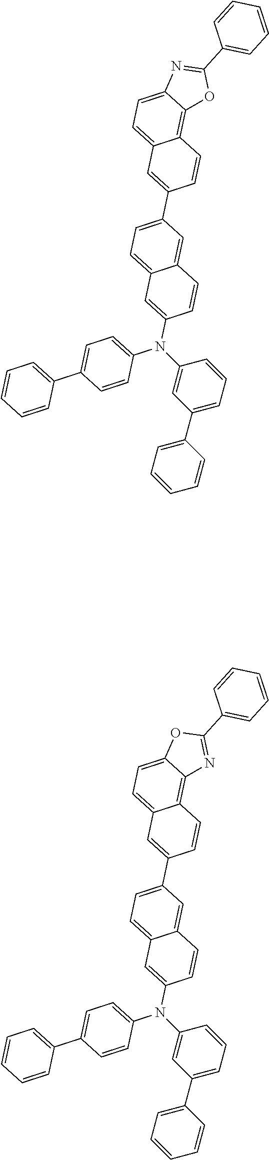 [SMILES: c1ccc(-c2ccc(N(c3cccc(-c4ccccc4)c3)c3ccc4cc(-c5ccc6c(ccc7nc(-c8ccccc8)oc76)c5)ccc4c3)cc2)cc1 ; c1ccc(-c2ccc(N(c3cccc(-c4ccccc4)c3)c3ccc4cc(-c5ccc6c(ccc7oc(-c8ccccc8)nc76)c5)ccc4c3)cc2)cc1]

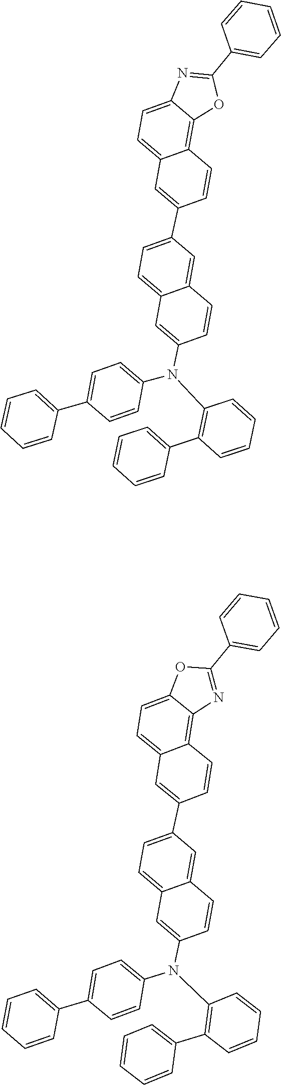 [SMILES: c1ccc(-c2ccc(N(c3ccc4cc(-c5ccc6c(ccc7nc(-c8ccccc8)oc76)c5)ccc4c3)c3ccccc3-c3ccccc3)cc2)cc1 ; c1ccc(-c2ccc(N(c3ccc4cc(-c5ccc6c(ccc7oc(-c8ccccc8)nc76)c5)ccc4c3)c3ccccc3-c3ccccc3)cc2)cc1]

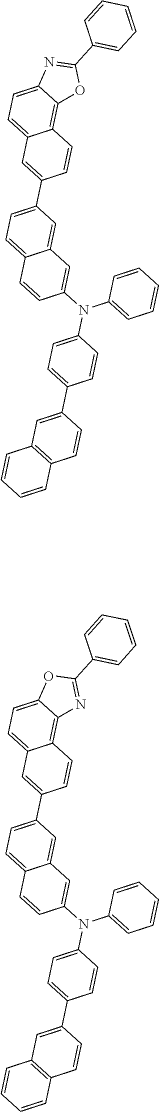 [SMILES: c1ccc(-c2nc3ccc4cc(-c5ccc6ccc(N(c7ccccc7)c7ccc(-c8ccc9ccccc9c8)cc7)cc6c5)ccc4c3o2)cc1 ; c1ccc(-c2nc3c(ccc4cc(-c5ccc6ccc(N(c7ccccc7)c7ccc(-c8ccc9ccccc9c8)cc7)cc6c5)ccc43)o2)cc1]

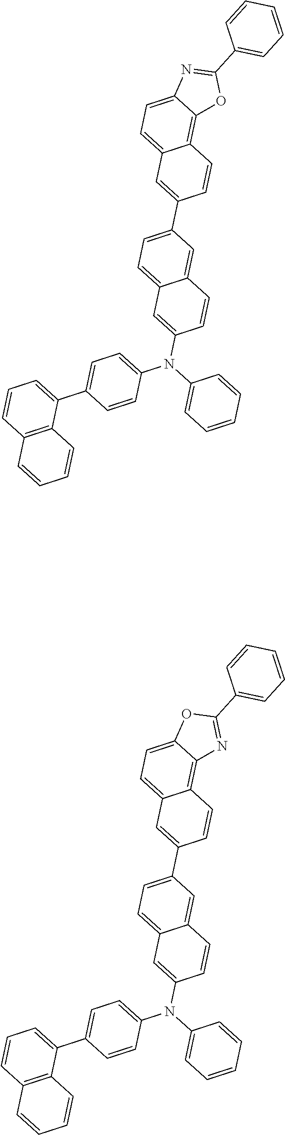 [SMILES: c1ccc(-c2nc3ccc4cc(-c5ccc6cc(N(c7ccccc7)c7ccc(-c8cccc9ccccc89)cc7)ccc6c5)ccc4c3o2)cc1 ; c1ccc(-c2nc3c(ccc4cc(-c5ccc6cc(N(c7ccccc7)c7ccc(-c8cccc9ccccc89)cc7)ccc6c5)ccc43)o2)cc1]

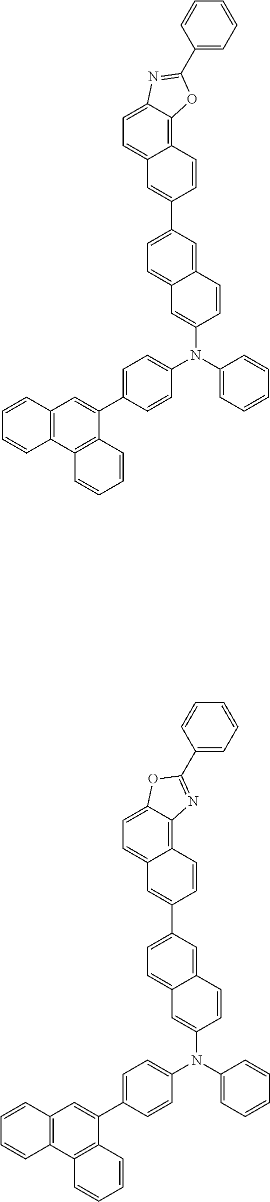 [SMILES: c1ccc(-c2nc3ccc4cc(-c5ccc6cc(N(c7ccccc7)c7ccc(-c8cc9ccccc9c9ccccc89)cc7)ccc6c5)ccc4c3o2)cc1 ; c1ccc(-c2nc3c(ccc4cc(-c5ccc6cc(N(c7ccccc7)c7ccc(-c8cc9ccccc9c9ccccc89)cc7)ccc6c5)ccc43)o2)cc1]

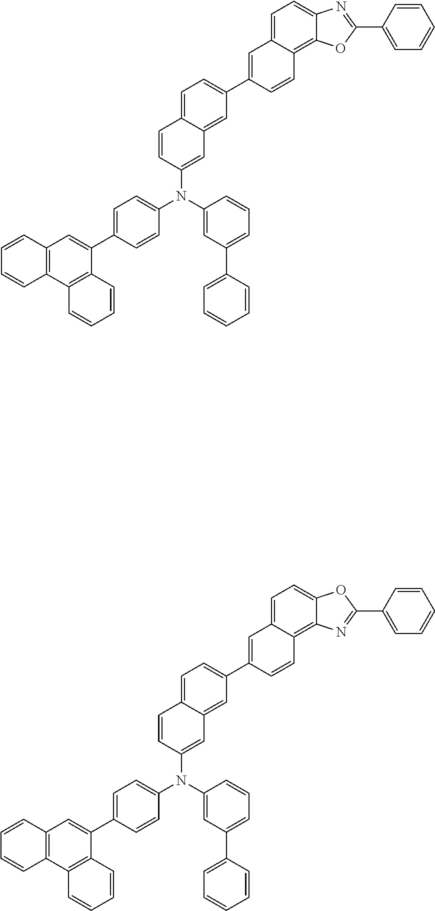 [SMILES: c1ccc(-c2cccc(N(c3ccc(-c4cc5ccccc5c5ccccc45)cc3)c3ccc4ccc(-c5ccc6c(ccc7nc(-c8ccccc8)oc76)c5)cc4c3)c2)cc1 ; c1ccc(-c2cccc(N(c3ccc(-c4cc5ccccc5c5ccccc45)cc3)c3ccc4ccc(-c5ccc6c(ccc7oc(-c8ccccc8)nc76)c5)cc4c3)c2)cc1]

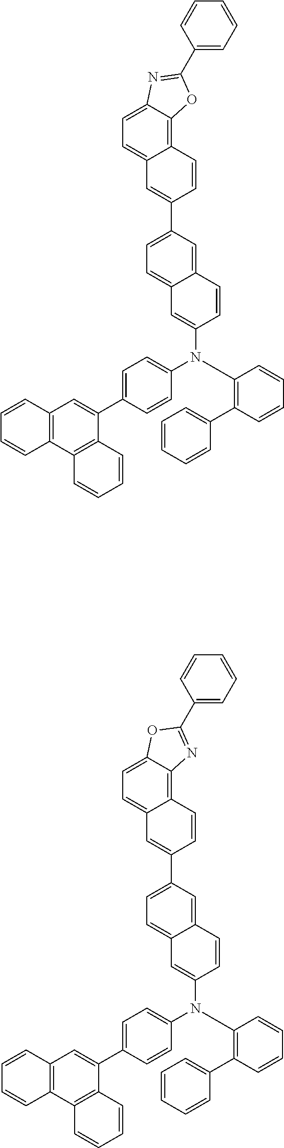 [SMILES: c1ccc(-c2nc3ccc4cc(-c5ccc6cc(N(c7ccc(-c8cc9ccccc9c9ccccc89)cc7)c7ccccc7-c7ccccc7)ccc6c5)ccc4c3o2)cc1 ; c1ccc(-c2nc3c(ccc4cc(-c5ccc6cc(N(c7ccc(-c8cc9ccccc9c9ccccc89)cc7)c7ccccc7-c7ccccc7)ccc6c5)ccc43)o2)cc1]

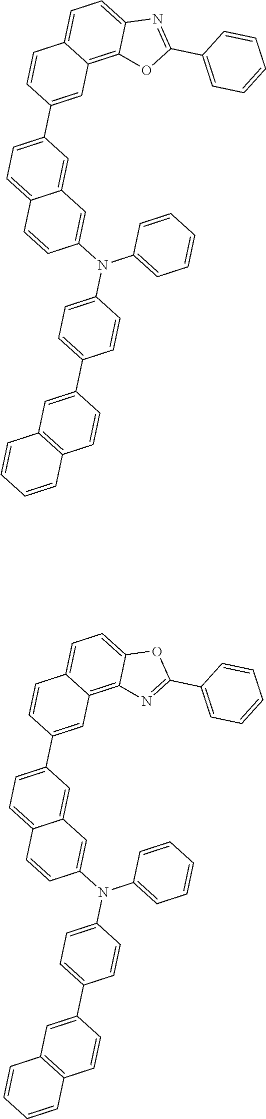 [SMILES: c1ccc(-c2nc3ccc4ccc(-c5ccc6ccc(N(c7ccccc7)c7ccc(-c8ccc9ccccc9c8)cc7)cc6c5)cc4c3o2)cc1 ; c1ccc(-c2nc3c(ccc4ccc(-c5ccc6ccc(N(c7ccccc7)c7ccc(-c8ccc9ccccc9c8)cc7)cc6c5)cc43)o2)cc1]

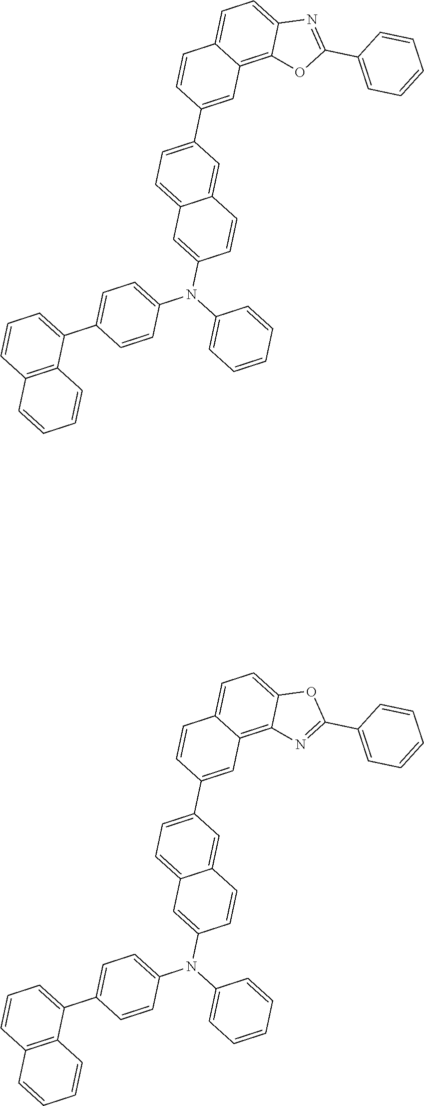 [SMILES: c1ccc(-c2nc3ccc4ccc(-c5ccc6cc(N(c7ccccc7)c7ccc(-c8cccc9ccccc89)cc7)ccc6c5)cc4c3o2)cc1 ; c1ccc(-c2nc3c(ccc4ccc(-c5ccc6cc(N(c7ccccc7)c7ccc(-c8cccc9ccccc89)cc7)ccc6c5)cc43)o2)cc1]

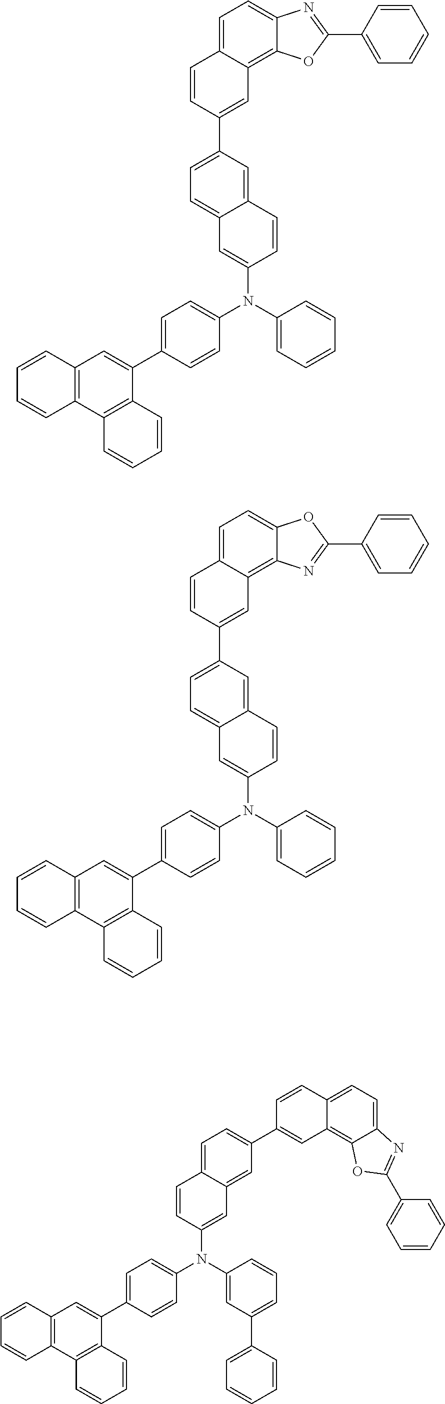 [SMILES: c1ccc(-c2nc3ccc4ccc(-c5ccc6cc(N(c7ccccc7)c7ccc(-c8cc9ccccc9c9ccccc89)cc7)ccc6c5)cc4c3o2)cc1 ; c1ccc(-c2nc3c(ccc4ccc(-c5ccc6cc(N(c7ccccc7)c7ccc(-c8cc9ccccc9c9ccccc89)cc7)ccc6c5)cc43)o2)cc1 ; c1ccc(-c2cccc(N(c3ccc(-c4cc5ccccc5c5ccccc45)cc3)c3ccc4ccc(-c5ccc6ccc7nc(-c8ccccc8)oc7c6c5)cc4c3)c2)cc1]

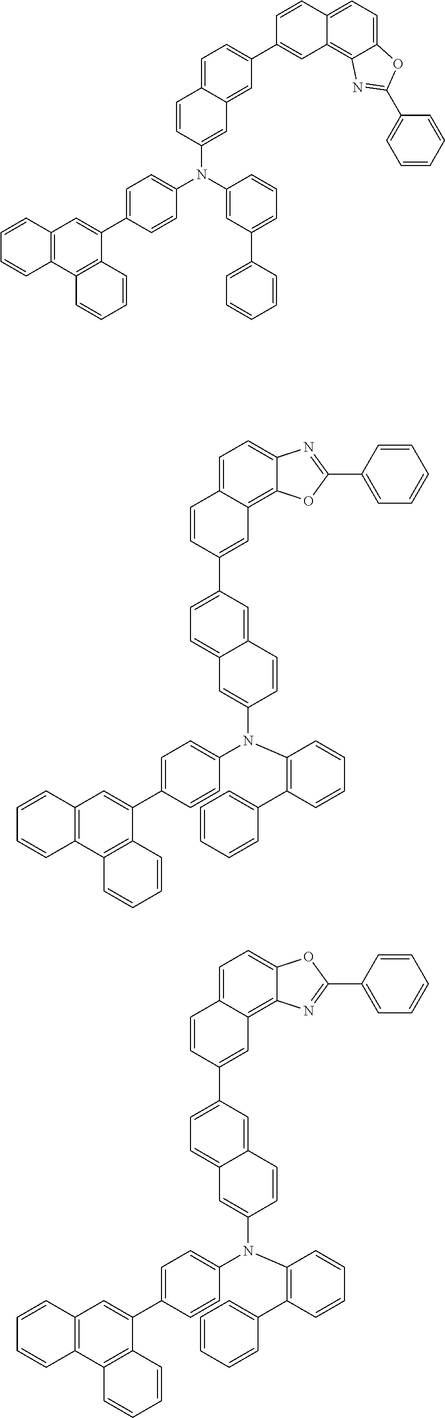 [SMILES: c1ccc(-c2cccc(N(c3ccc(-c4cc5ccccc5c5ccccc45)cc3)c3ccc4ccc(-c5ccc6ccc7oc(-c8ccccc8)nc7c6c5)cc4c3)c2)cc1 ; c1ccc(-c2nc3ccc4ccc(-c5ccc6cc(N(c7ccc(-c8cc9ccccc9c9ccccc89)cc7)c7ccccc7-c7ccccc7)ccc6c5)cc4c3o2)cc1 ; c1ccc(-c2nc3c(ccc4ccc(-c5ccc6cc(N(c7ccc(-c8cc9ccccc9c9ccccc89)cc7)c7ccccc7-c7ccccc7)ccc6c5)cc43)o2)cc1]

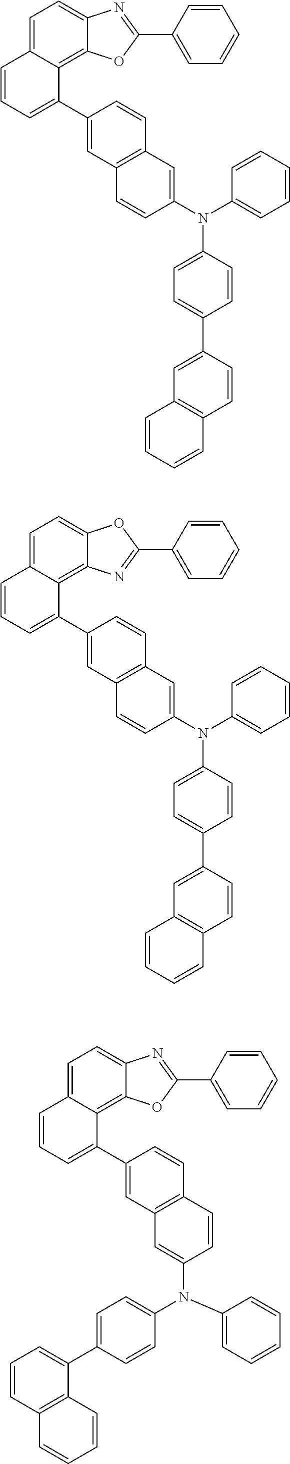 [SMILES: c1ccc(-c2nc3ccc4cccc(-c5ccc6cc(N(c7ccccc7)c7ccc(-c8ccc9ccccc9c8)cc7)ccc6c5)c4c3o2)cc1 ; c1ccc(-c2nc3c(ccc4cccc(-c5ccc6cc(N(c7ccccc7)c7ccc(-c8ccc9ccccc9c8)cc7)ccc6c5)c43)o2)cc1 ; c1ccc(-c2nc3ccc4cccc(-c5ccc6ccc(N(c7ccccc7)c7ccc(-c8cccc9ccccc89)cc7)cc6c5)c4c3o2)cc1]

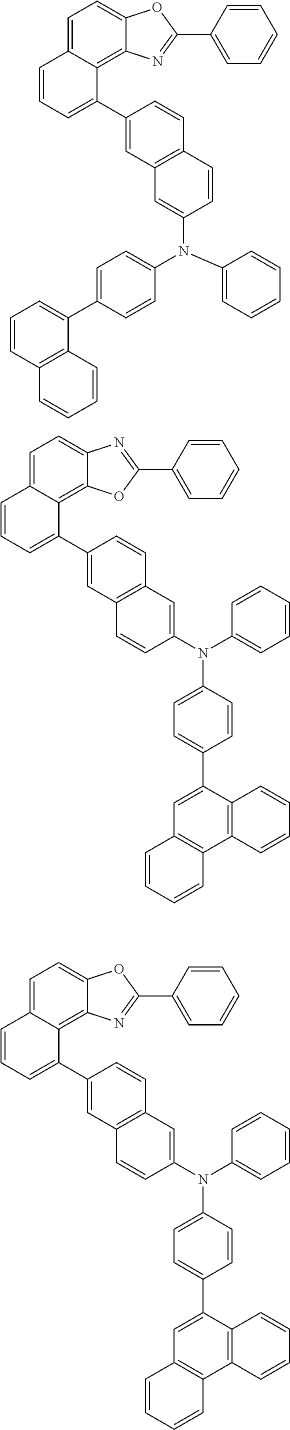

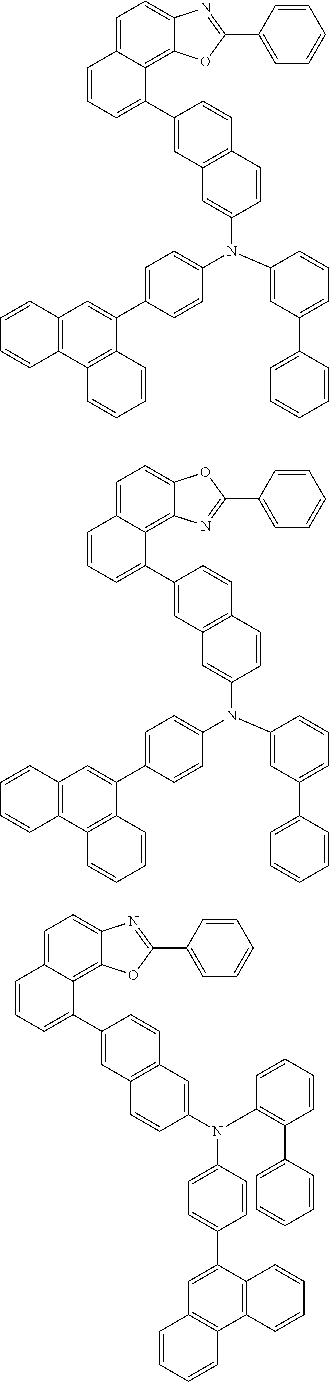

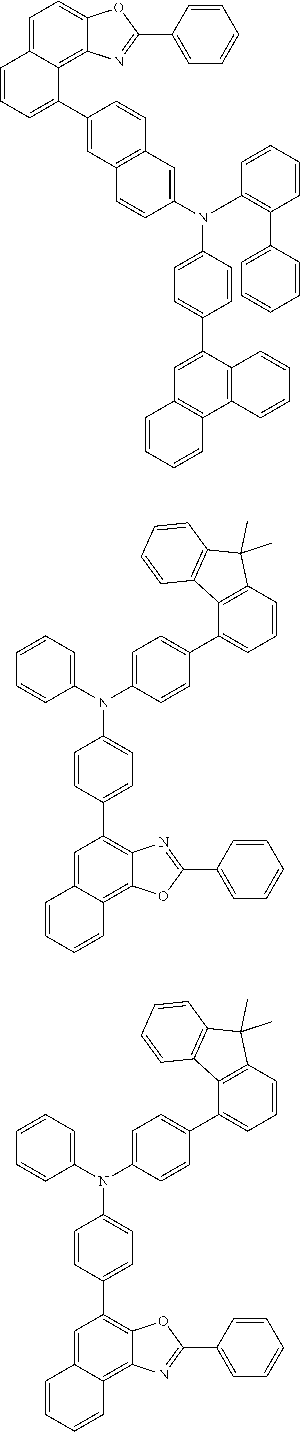 [SMILES: c1ccc(-c2nc3c(ccc4cccc(-c5ccc6cc(N(c7ccc(-c8cc9ccccc9c9ccccc89)cc7)c7ccccc7-c7ccccc7)ccc6c5)c43)o2)cc1 ; CC1(C)c2ccccc2-c2c(-c3ccc(N(c4ccccc4)c4ccc(-c5cc6ccccc6c6oc(-c7ccccc7)nc56)cc4)cc3)cccc21 ; CC1(C)c2ccccc2-c2c(-c3ccc(N(c4ccccc4)c4ccc(-c5cc6ccccc6c6nc(-c7ccccc7)oc56)cc4)cc3)cccc21]

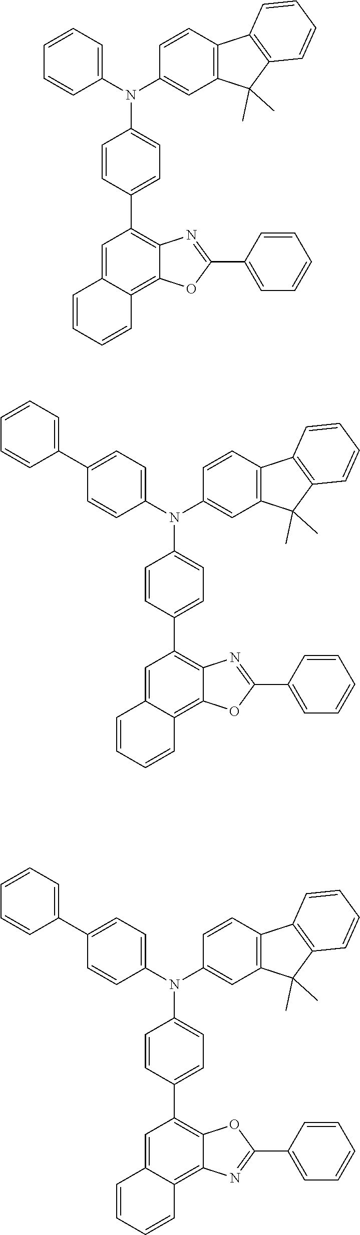 [SMILES: CC1(C)c2ccccc2-c2ccc(N(c3ccccc3)c3ccc(-c4cc5ccccc5c5oc(-c6ccccc6)nc45)cc3)cc21 ; CC1(C)c2ccccc2-c2ccc(N(c3ccc(-c4ccccc4)cc3)c3ccc(-c4cc5ccccc5c5oc(-c6ccccc6)nc45)cc3)cc21 ; CC1(C)c2ccccc2-c2ccc(N(c3ccc(-c4ccccc4)cc3)c3ccc(-c4cc5ccccc5c5nc(-c6ccccc6)oc45)cc3)cc21]

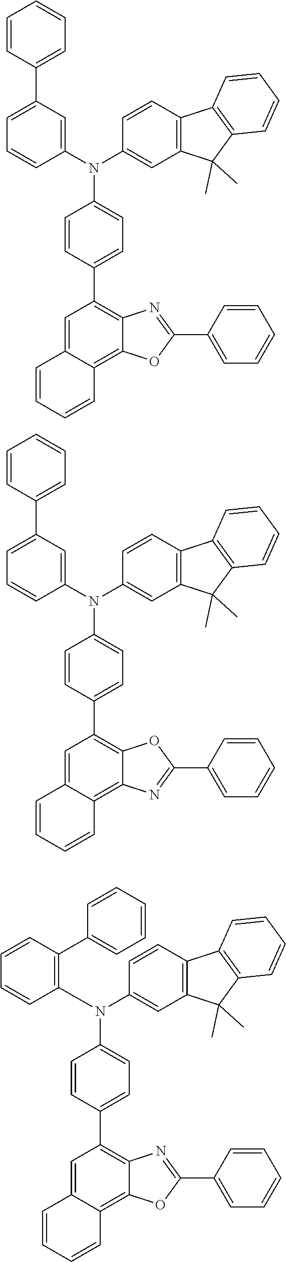

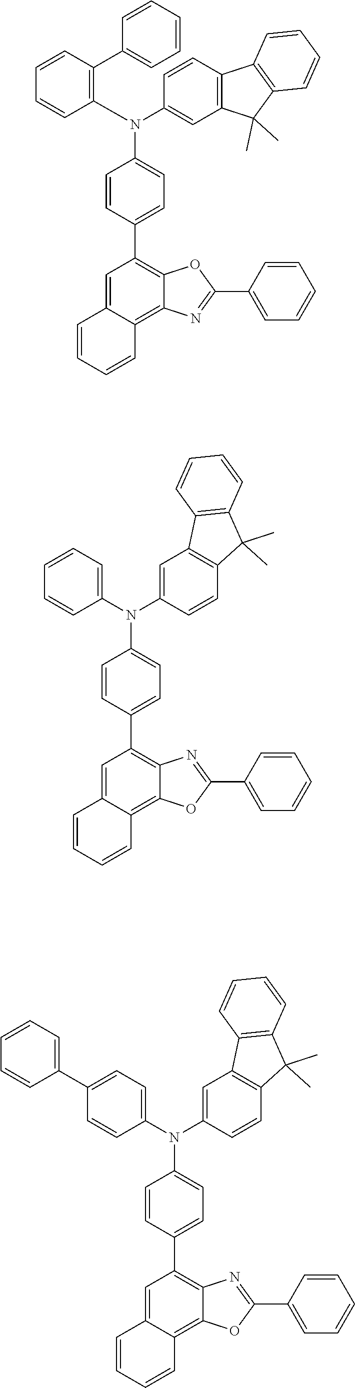 [SMILES: CC1(C)c2ccccc2-c2ccc(N(c3ccc(-c4cc5ccccc5c5nc(-c6ccccc6)oc45)cc3)c3ccccc3-c3ccccc3)cc21 ; CC1(C)c2ccccc2-c2cc(N(c3ccccc3)c3ccc(-c4cc5ccccc5c5oc(-c6ccccc6)nc45)cc3)ccc21 ; CC1(C)c2ccccc2-c2cc(N(c3ccc(-c4ccccc4)cc3)c3ccc(-c4cc5ccccc5c5oc(-c6ccccc6)nc45)cc3)ccc21]

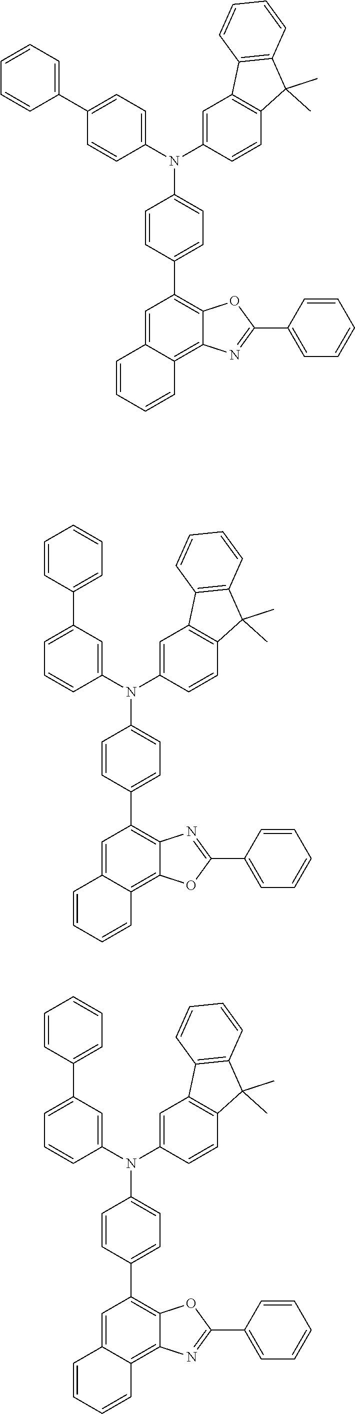 [SMILES: CC1(C)c2ccccc2-c2cc(N(c3ccc(-c4ccccc4)cc3)c3ccc(-c4cc5ccccc5c5nc(-c6ccccc6)oc45)cc3)ccc21 ; CC1(C)c2ccccc2-c2cc(N(c3ccc(-c4cc5ccccc5c5oc(-c6ccccc6)nc45)cc3)c3cccc(-c4ccccc4)c3)ccc21 ; CC1(C)c2ccccc2-c2cc(N(c3ccc(-c4cc5ccccc5c5nc(-c6ccccc6)oc45)cc3)c3cccc(-c4ccccc4)c3)ccc21]

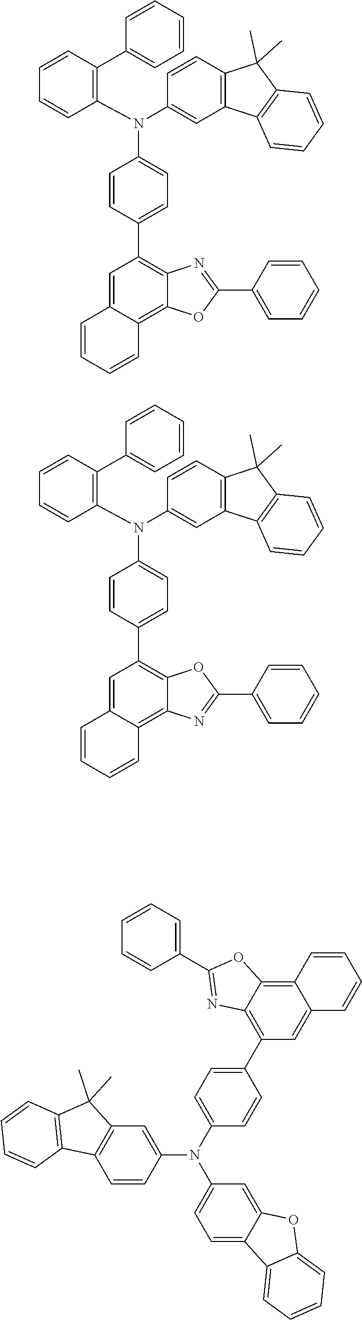 [SMILES: CC1(C)c2ccccc2-c2cc(N(c3ccc(-c4cc5ccccc5c5oc(-c6ccccc6)nc45)cc3)c3ccccc3-c3ccccc3)ccc21 ; CC1(C)c2ccccc2-c2cc(N(c3ccc(-c4cc5ccccc5c5nc(-c6ccccc6)oc45)cc3)c3ccccc3-c3ccccc3)ccc21 ; CC1(C)c2ccccc2-c2ccc(N(c3ccc(-c4cc5ccccc5c5oc(-c6ccccc6)nc45)cc3)c3ccc4c(c3)oc3ccccc34)cc21]

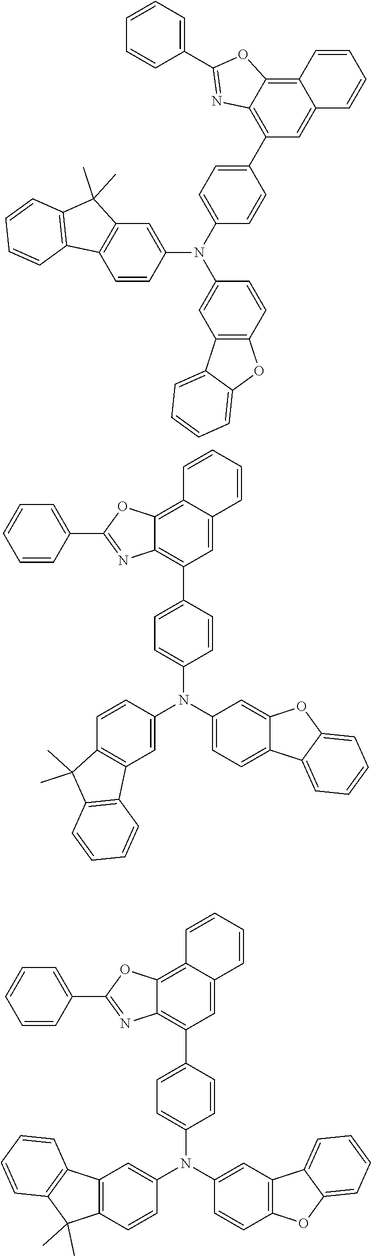

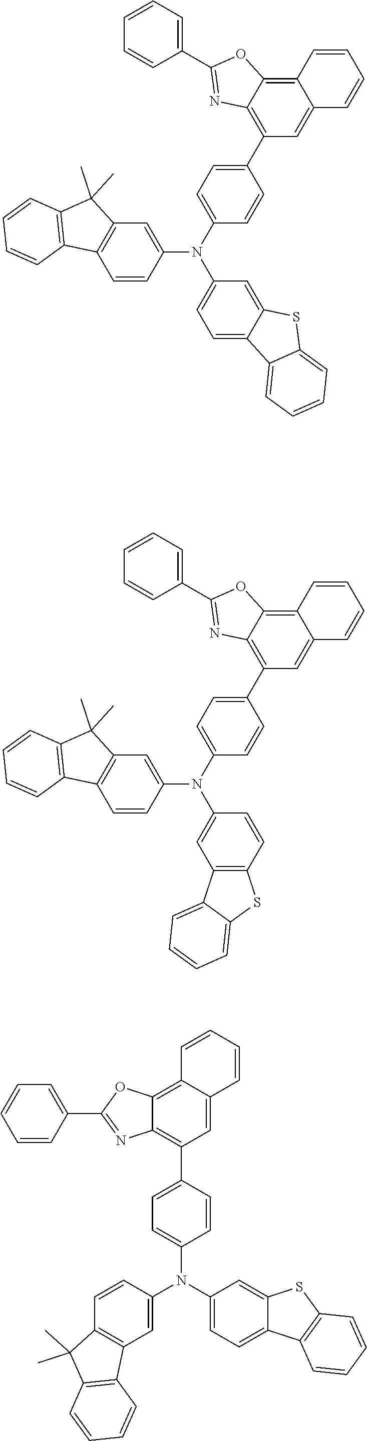 [SMILES: CC1(C)c2ccccc2-c2ccc(N(c3ccc(-c4cc5ccccc5c5oc(-c6ccccc6)nc45)cc3)c3ccc4c(c3)sc3ccccc34)cc21 ; CC1(C)c2ccccc2-c2ccc(N(c3ccc(-c4cc5ccccc5c5oc(-c6ccccc6)nc45)cc3)c3ccc4sc5ccccc5c4c3)cc21 ; CC1(C)c2ccccc2-c2cc(N(c3ccc(-c4cc5ccccc5c5oc(-c6ccccc6)nc45)cc3)c3ccc4c(c3)sc3ccccc34)ccc21]

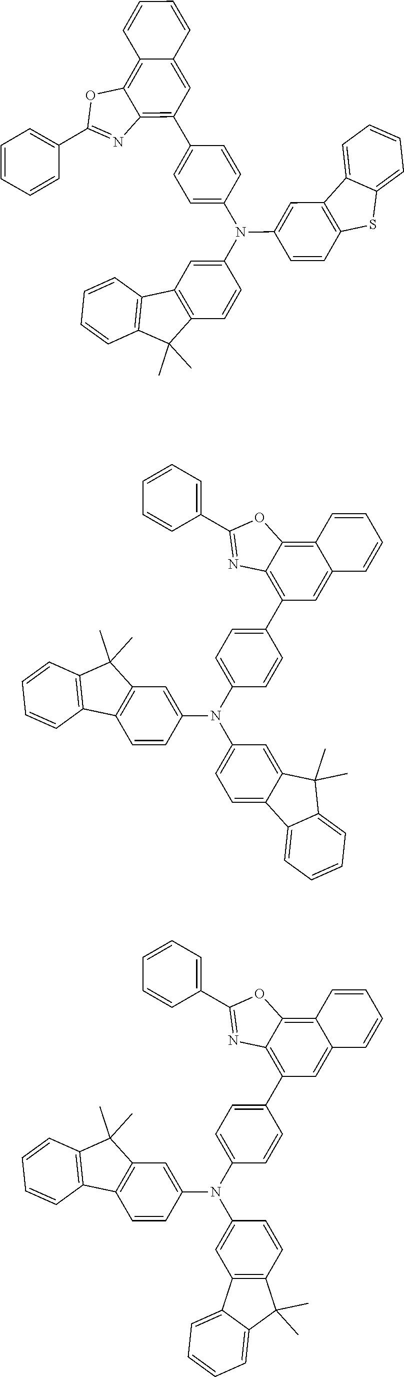 [SMILES: CC1(C)c2ccccc2-c2cc(N(c3ccc(-c4cc5ccccc5c5oc(-c6ccccc6)nc45)cc3)c3ccc4sc5ccccc5c4c3)ccc21 ; CC1(C)c2ccccc2-c2ccc(N(c3ccc(-c4cc5ccccc5c5oc(-c6ccccc6)nc45)cc3)c3ccc4c(c3)C(C)(C)c3ccccc3-4)cc21 ; CC1(C)c2ccccc2-c2cc(N(c3ccc(-c4cc5ccccc5c5oc(-c6ccccc6)nc45)cc3)c3ccc4c(c3)C(C)(C)c3ccccc3-4)ccc21]

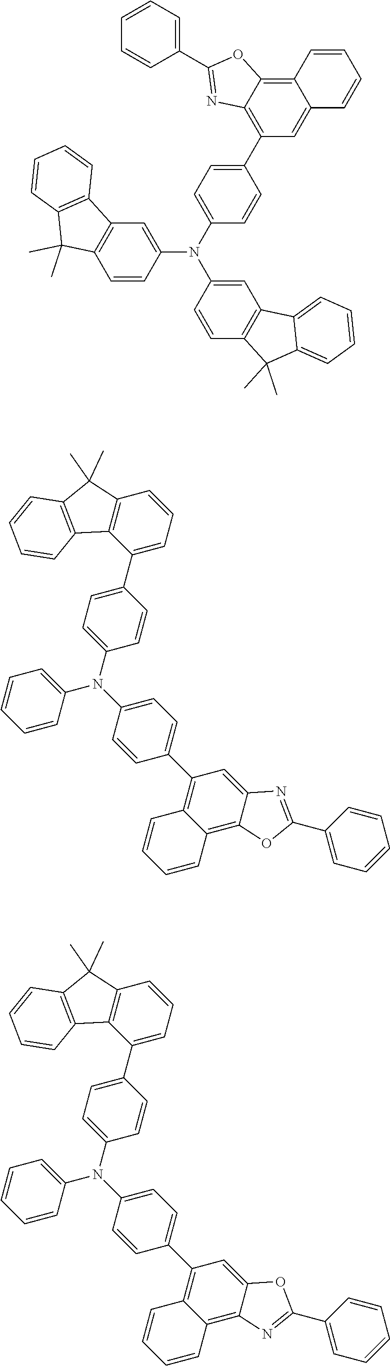 [SMILES: CC1(C)c2ccccc2-c2cc(N(c3ccc(-c4cc5ccccc5c5oc(-c6ccccc6)nc45)cc3)c3ccc4c(c3)-c3ccccc3C4(C)C)ccc21 ; CC1(C)c2ccccc2-c2c(-c3ccc(N(c4ccccc4)c4ccc(-c5cc6nc(-c7ccccc7)oc6c6ccccc56)cc4)cc3)cccc21 ; CC1(C)c2ccccc2-c2c(-c3ccc(N(c4ccccc4)c4ccc(-c5cc6oc(-c7ccccc7)nc6c6ccccc56)cc4)cc3)cccc21]

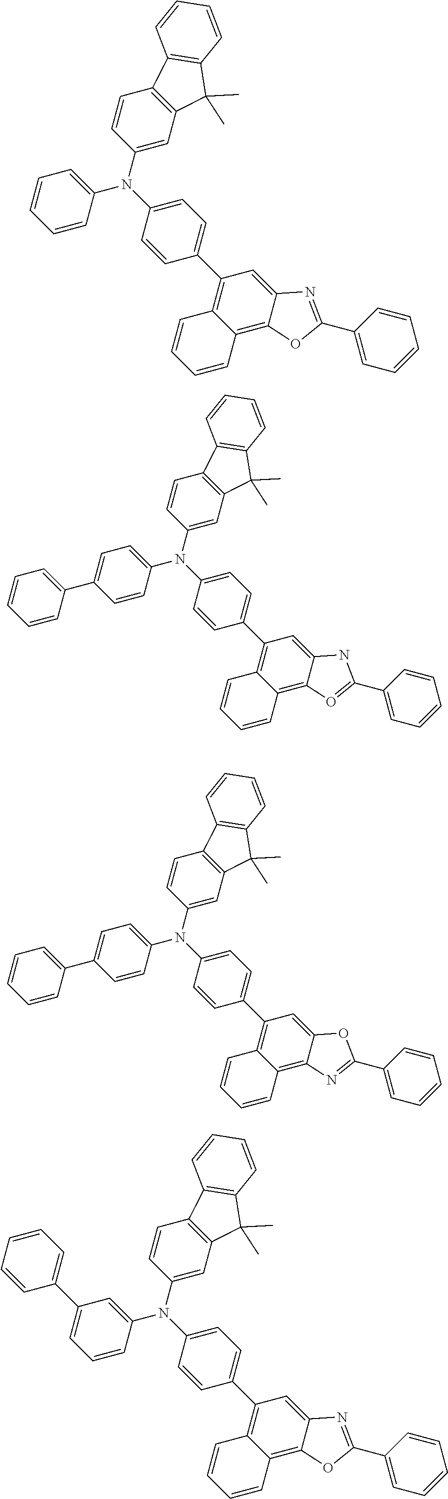

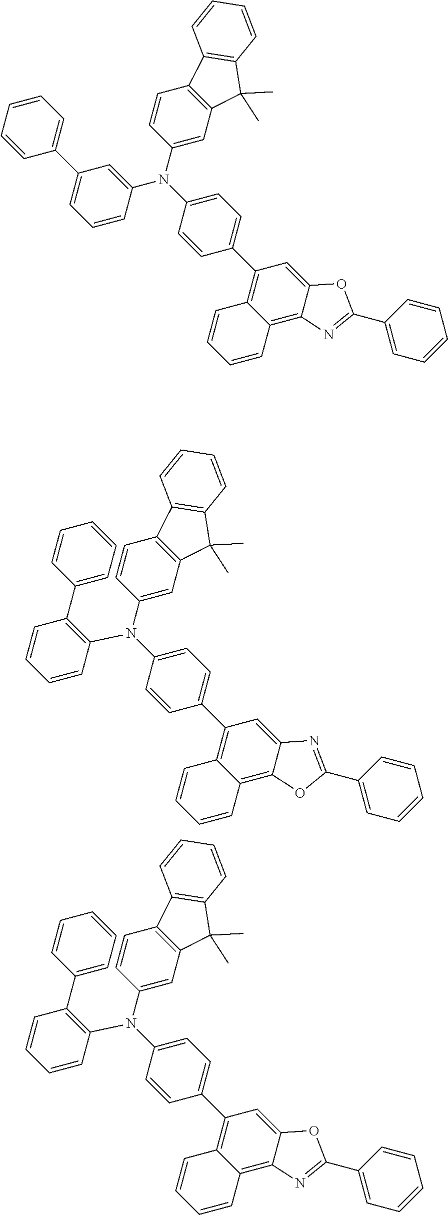

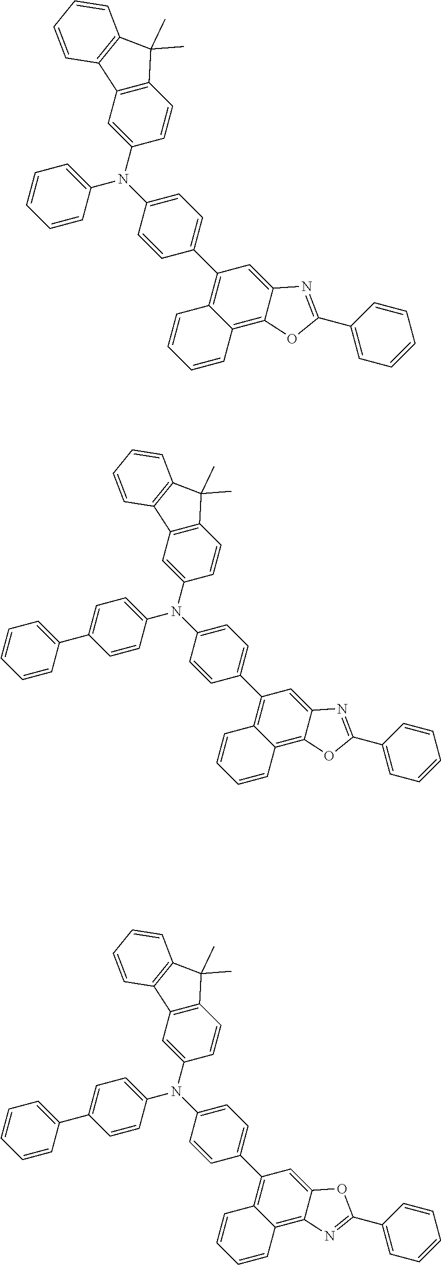 [SMILES: CC1(C)c2ccccc2-c2cc(N(c3ccccc3)c3ccc(-c4cc5nc(-c6ccccc6)oc5c5ccccc45)cc3)ccc21 ; CC1(C)c2ccccc2-c2cc(N(c3ccc(-c4ccccc4)cc3)c3ccc(-c4cc5nc(-c6ccccc6)oc5c5ccccc45)cc3)ccc21 ; CC1(C)c2ccccc2-c2cc(N(c3ccc(-c4ccccc4)cc3)c3ccc(-c4cc5oc(-c6ccccc6)nc5c5ccccc45)cc3)ccc21]

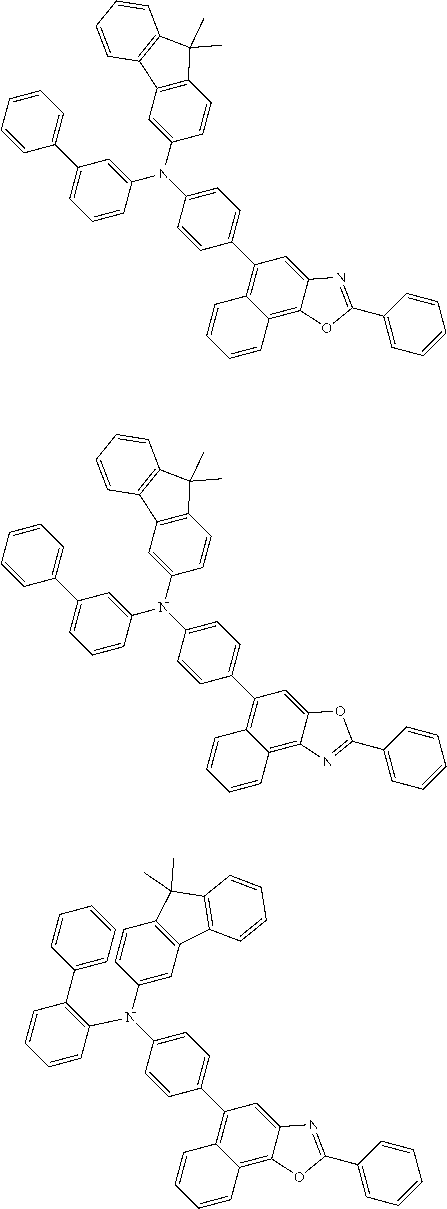 [SMILES: CC1(C)c2ccccc2-c2cc(N(c3ccc(-c4cc5nc(-c6ccccc6)oc5c5ccccc45)cc3)c3cccc(-c4ccccc4)c3)ccc21 ; CC1(C)c2ccccc2-c2cc(N(c3ccc(-c4cc5oc(-c6ccccc6)nc5c5ccccc45)cc3)c3cccc(-c4ccccc4)c3)ccc21 ; CC1(C)c2ccccc2-c2cc(N(c3ccc(-c4cc5nc(-c6ccccc6)oc5c5ccccc45)cc3)c3ccccc3-c3ccccc3)ccc21]

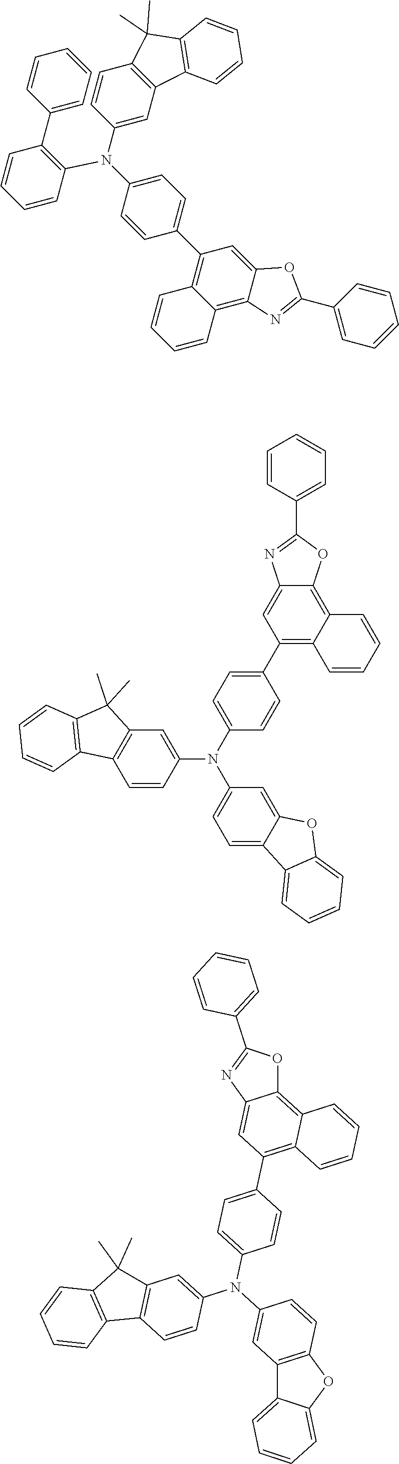 [SMILES: CC1(C)c2ccccc2-c2cc(N(c3ccc(-c4cc5oc(-c6ccccc6)nc5c5ccccc45)cc3)c3ccccc3-c3ccccc3)ccc21 ; CC1(C)c2ccccc2-c2ccc(N(c3ccc(-c4cc5nc(-c6ccccc6)oc5c5ccccc45)cc3)c3ccc4c(c3)oc3ccccc34)cc21 ; CC1(C)c2ccccc2-c2ccc(N(c3ccc(-c4cc5nc(-c6ccccc6)oc5c5ccccc45)cc3)c3ccc4oc5ccccc5c4c3)cc21]

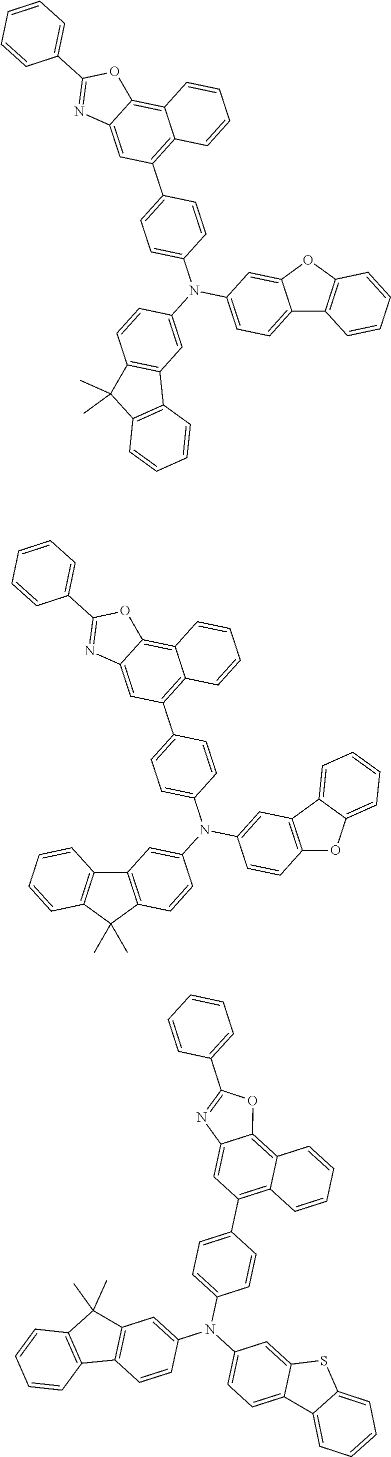 [SMILES: CC1(C)c2ccccc2-c2cc(N(c3ccc(-c4cc5nc(-c6ccccc6)oc5c5ccccc45)cc3)c3ccc4c(c3)oc3ccccc34)ccc21 ; CC1(C)c2ccccc2-c2cc(N(c3ccc(-c4cc5nc(-c6ccccc6)oc5c5ccccc45)cc3)c3ccc4oc5ccccc5c4c3)ccc21 ; CC1(C)c2ccccc2-c2ccc(N(c3ccc(-c4cc5nc(-c6ccccc6)oc5c5ccccc45)cc3)c3ccc4c(c3)sc3ccccc34)cc21]

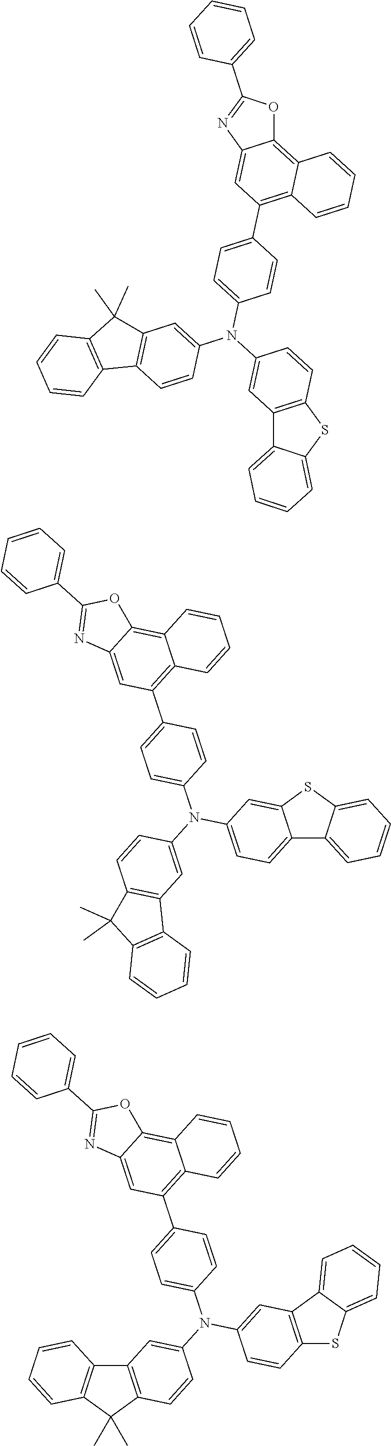 [SMILES: CC1(C)c2ccccc2-c2ccc(N(c3ccc(-c4cc5nc(-c6ccccc6)oc5c5ccccc45)cc3)c3ccc4sc5ccccc5c4c3)cc21 ; CC1(C)c2ccccc2-c2cc(N(c3ccc(-c4cc5nc(-c6ccccc6)oc5c5ccccc45)cc3)c3ccc4c(c3)sc3ccccc34)ccc21 ; CC1(C)c2ccccc2-c2cc(N(c3ccc(-c4cc5nc(-c6ccccc6)oc5c5ccccc45)cc3)c3ccc4sc5ccccc5c4c3)ccc21]

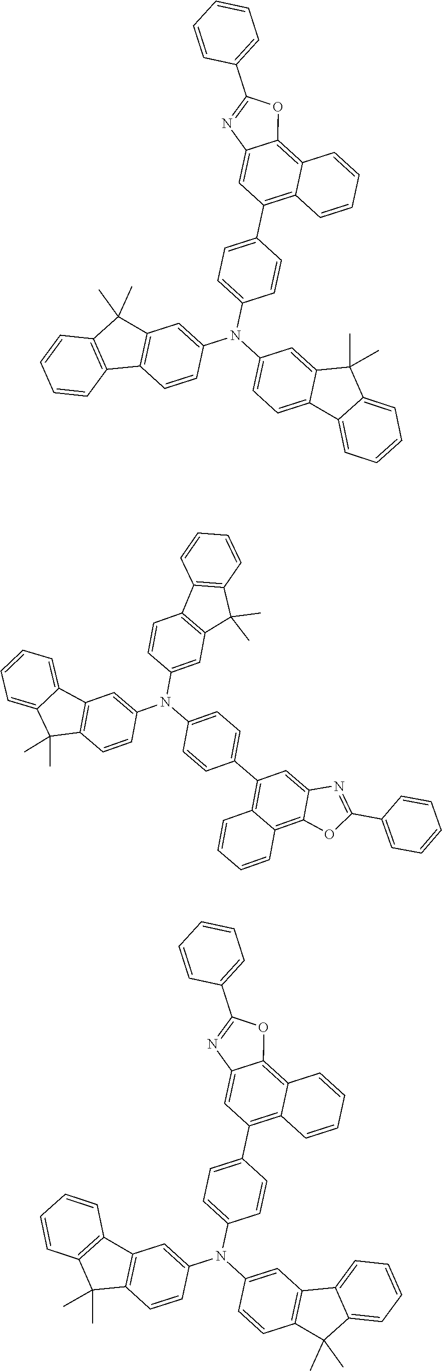 [SMILES: CC1(C)c2ccccc2-c2ccc(N(c3ccc(-c4cc5nc(-c6ccccc6)oc5c5ccccc45)cc3)c3ccc4c(c3)C(C)(C)c3ccccc3-4)cc21 ; CC1(C)c2ccccc2-c2cc(N(c3ccc(-c4cc5nc(-c6ccccc6)oc5c5ccccc45)cc3)c3ccc4c(c3)C(C)(C)c3ccccc3-4)ccc21 ; CC1(C)c2ccccc2-c2cc(N(c3ccc(-c4cc5nc(-c6ccccc6)oc5c5ccccc45)cc3)c3ccc4c(c3)-c3ccccc3C4(C)C)ccc21]

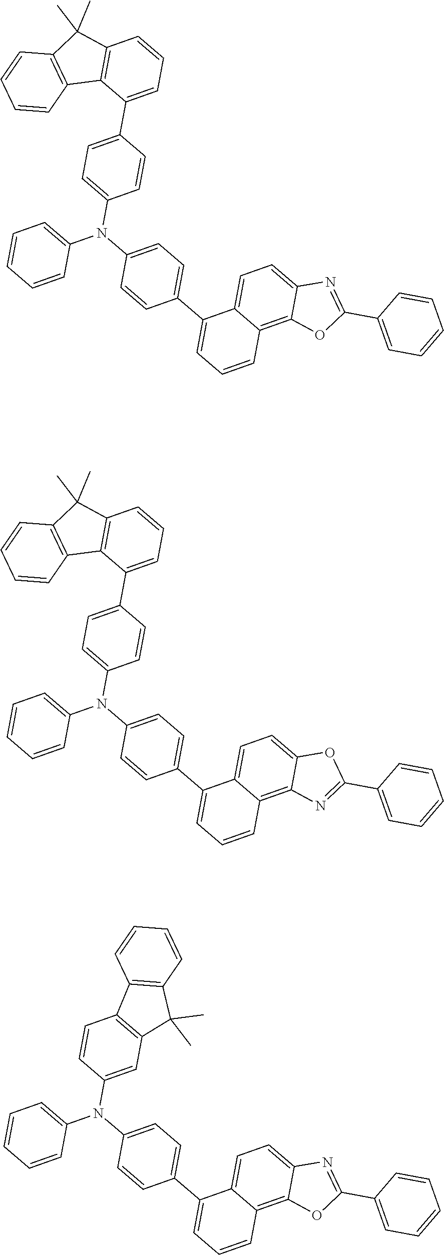 [SMILES: CC1(C)c2ccccc2-c2c(-c3ccc(N(c4ccccc4)c4ccc(-c5cccc6c5ccc5nc(-c7ccccc7)oc56)cc4)cc3)cccc21 ; CC1(C)c2ccccc2-c2c(-c3ccc(N(c4ccccc4)c4ccc(-c5cccc6c5ccc5oc(-c7ccccc7)nc56)cc4)cc3)cccc21 ; CC1(C)c2ccccc2-c2ccc(N(c3ccccc3)c3ccc(-c4cccc5c4ccc4nc(-c6ccccc6)oc45)cc3)cc21]

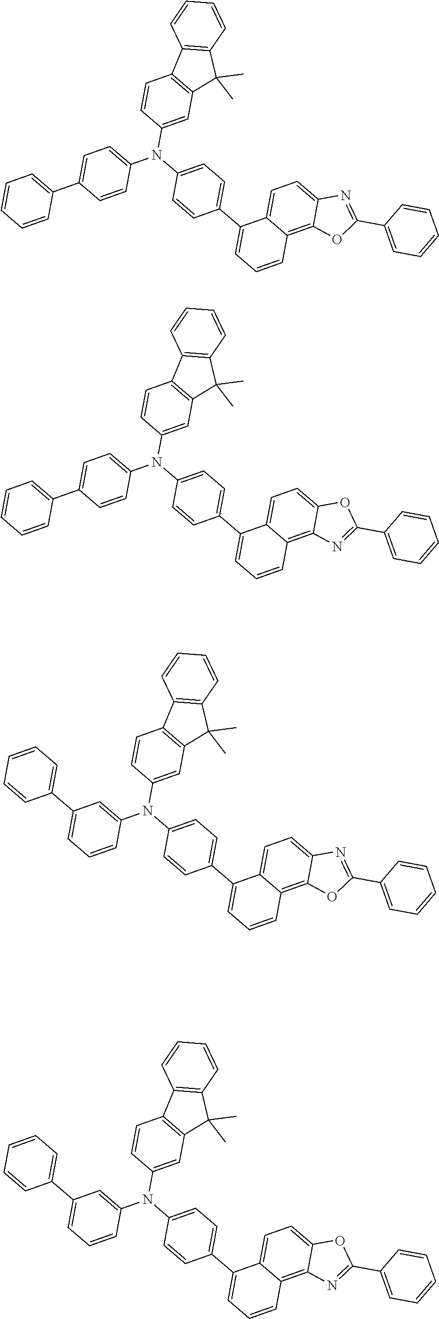 [SMILES: CC1(C)c2ccccc2-c2ccc(N(c3ccc(-c4ccccc4)cc3)c3ccc(-c4cccc5c4ccc4nc(-c6ccccc6)oc45)cc3)cc21 ; CC1(C)c2ccccc2-c2ccc(N(c3ccc(-c4ccccc4)cc3)c3ccc(-c4cccc5c4ccc4oc(-c6ccccc6)nc45)cc3)cc21 ; CC1(C)c2ccccc2-c2ccc(N(c3ccc(-c4cccc5c4ccc4nc(-c6ccccc6)oc45)cc3)c3cccc(-c4ccccc4)c3)cc21 ; CC1(C)c2ccccc2-c2ccc(N(c3ccc(-c4cccc5c4ccc4oc(-c6ccccc6)nc45)cc3)c3cccc(-c4ccccc4)c3)cc21]

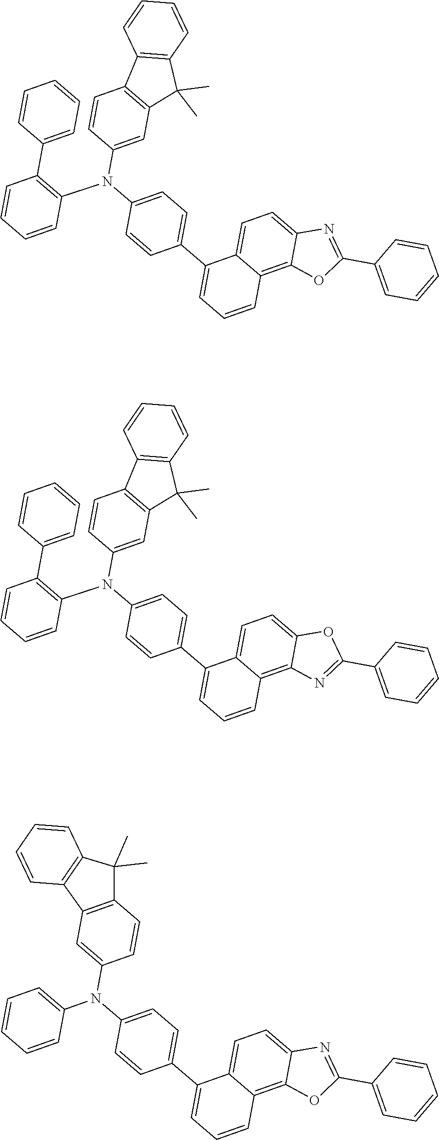 [SMILES: CC1(C)c2ccccc2-c2ccc(N(c3ccc(-c4cccc5c4ccc4nc(-c6ccccc6)oc45)cc3)c3ccccc3-c3ccccc3)cc21 ; CC1(C)c2ccccc2-c2ccc(N(c3ccc(-c4cccc5c4ccc4oc(-c6ccccc6)nc45)cc3)c3ccccc3-c3ccccc3)cc21 ; CC1(C)c2ccccc2-c2cc(N(c3ccccc3)c3ccc(-c4cccc5c4ccc4nc(-c6ccccc6)oc45)cc3)ccc21]

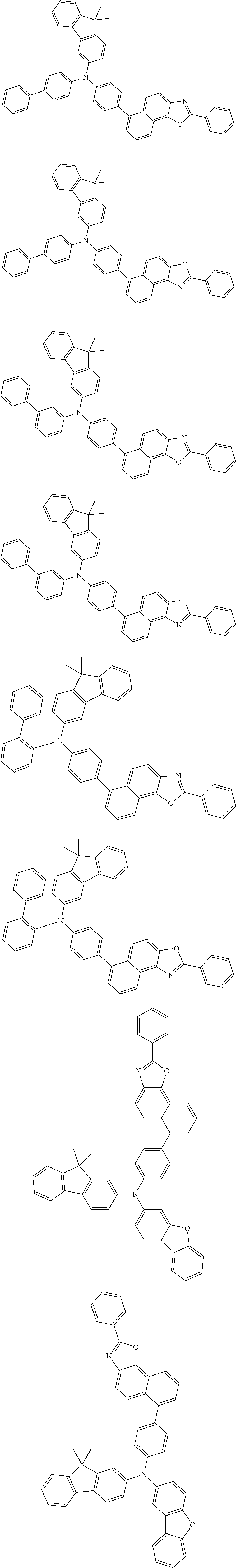

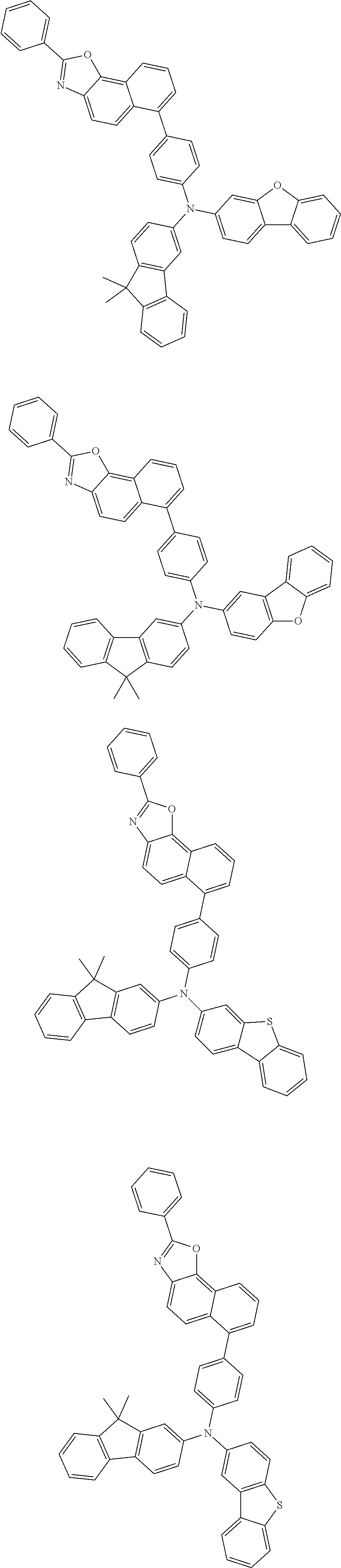

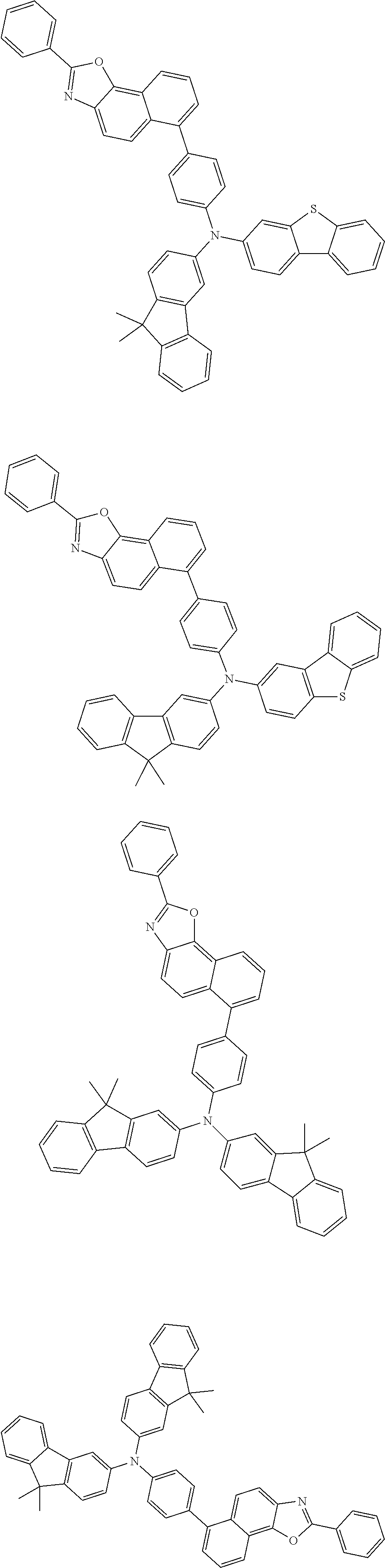

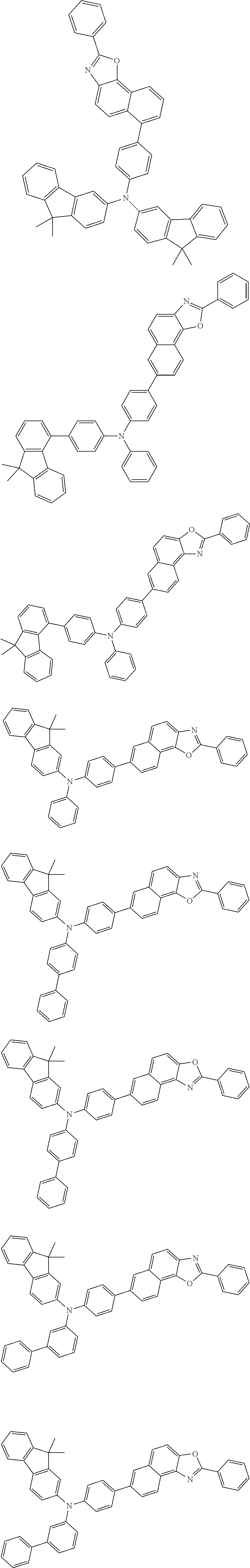

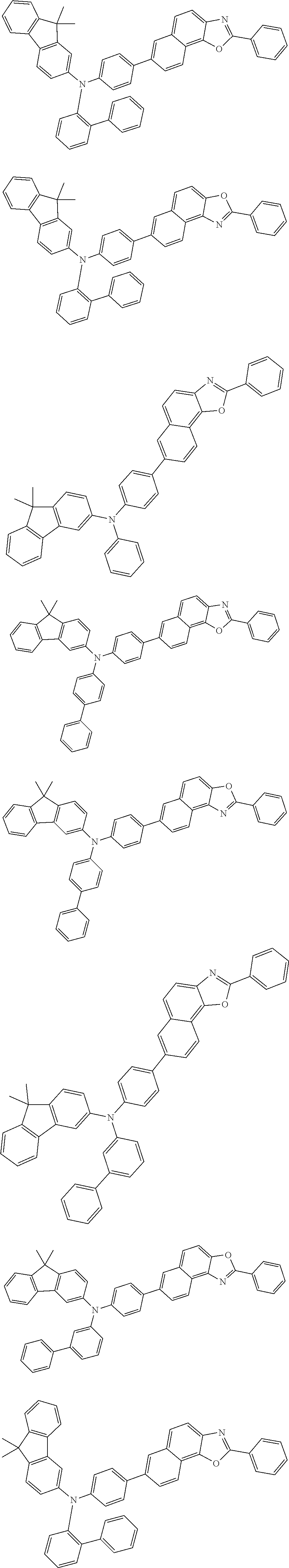

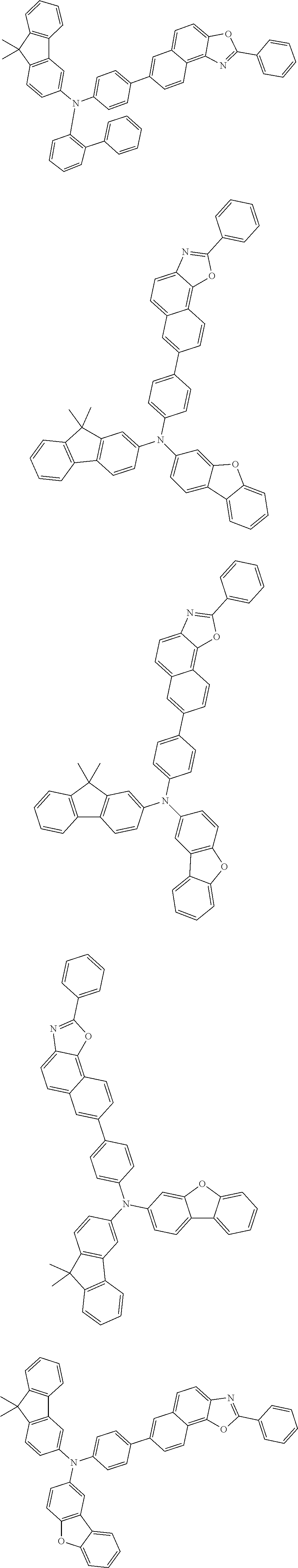 [SMILES: CC1(C)c2ccccc2-c2cc(N(c3ccc(-c4ccc5c(ccc6oc(-c7ccccc7)nc65)c4)cc3)c3ccccc3-c3ccccc3)ccc21 ; CC1(C)c2ccccc2-c2ccc(N(c3ccc(-c4ccc5c(ccc6nc(-c7ccccc7)oc65)c4)cc3)c3ccc4c(c3)oc3ccccc34)cc21 ; CC1(C)c2ccccc2-c2ccc(N(c3ccc(-c4ccc5c(ccc6nc(-c7ccccc7)oc65)c4)cc3)c3ccc4oc5ccccc5c4c3)cc21 ; CC1(C)c2ccccc2-c2cc(N(c3ccc(-c4ccc5c(ccc6nc(-c7ccccc7)oc65)c4)cc3)c3ccc4c(c3)oc3ccccc34)ccc21 ; CC1(C)c2ccccc2-c2cc(N(c3ccc(-c4ccc5c(ccc6nc(-c7ccccc7)oc65)c4)cc3)c3ccc4oc5ccccc5c4c3)ccc21]

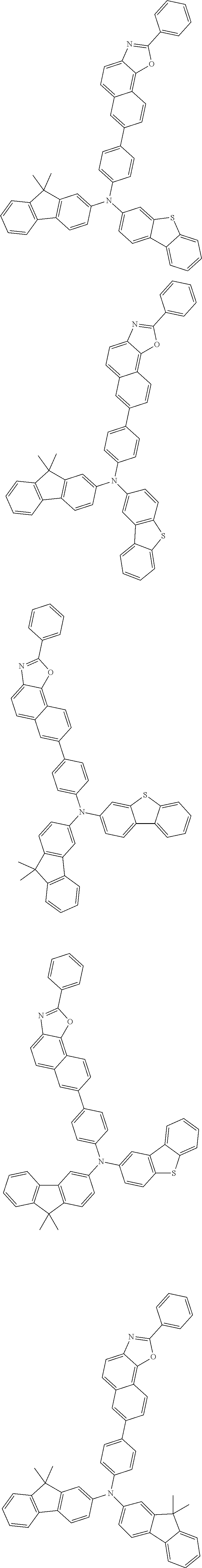

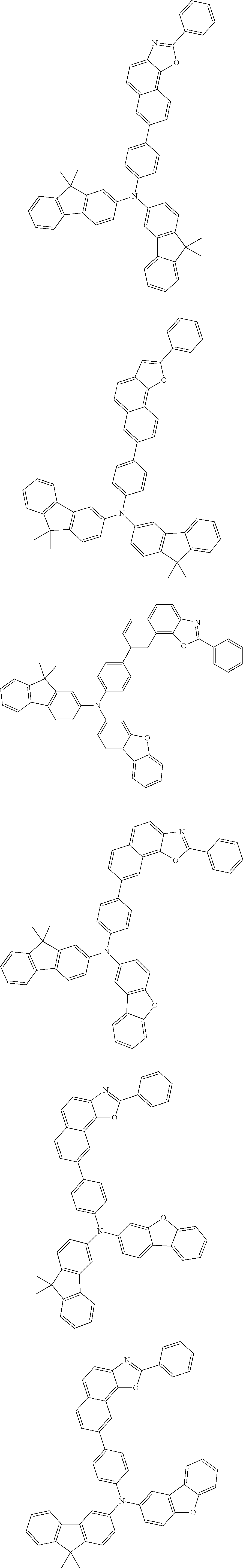

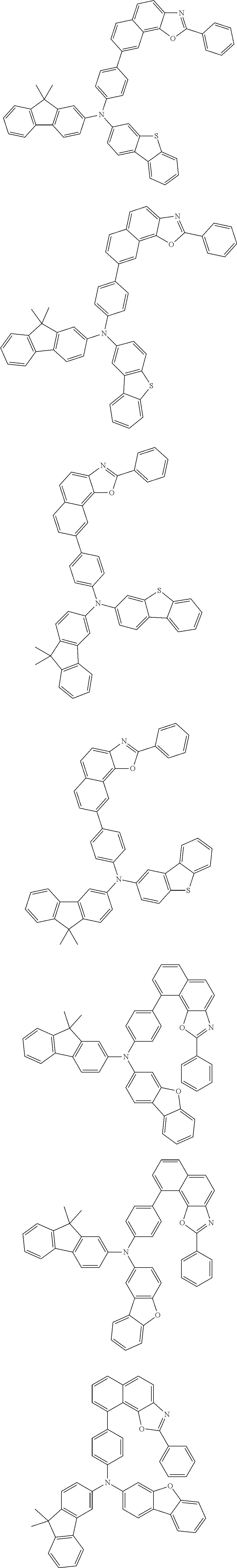

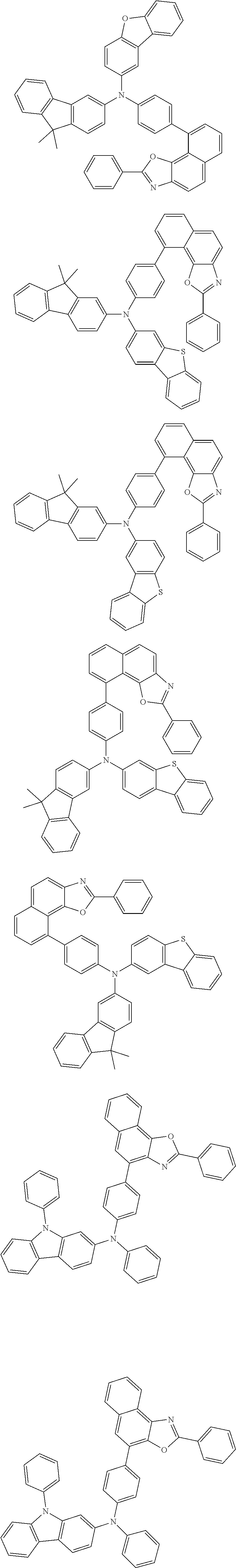

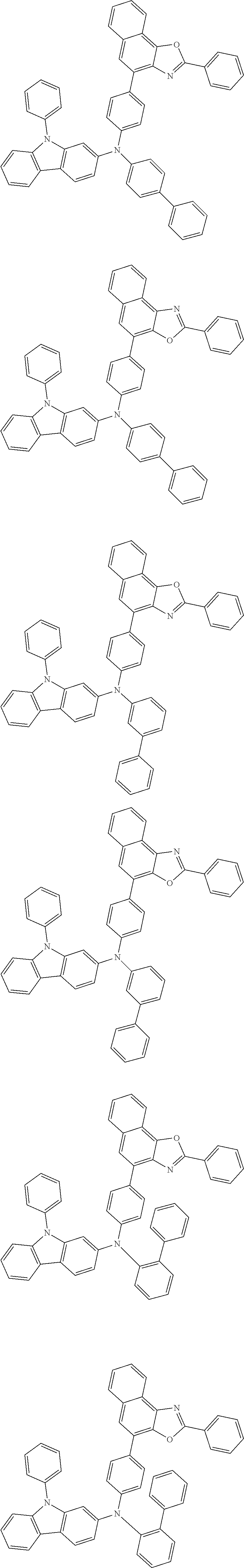

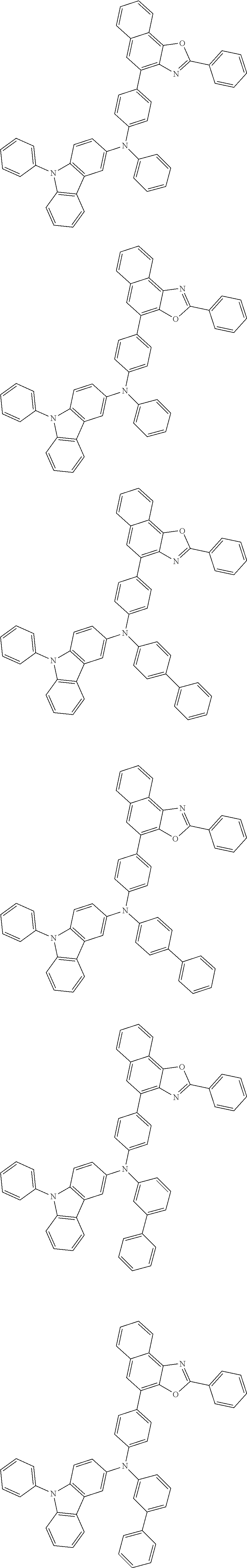

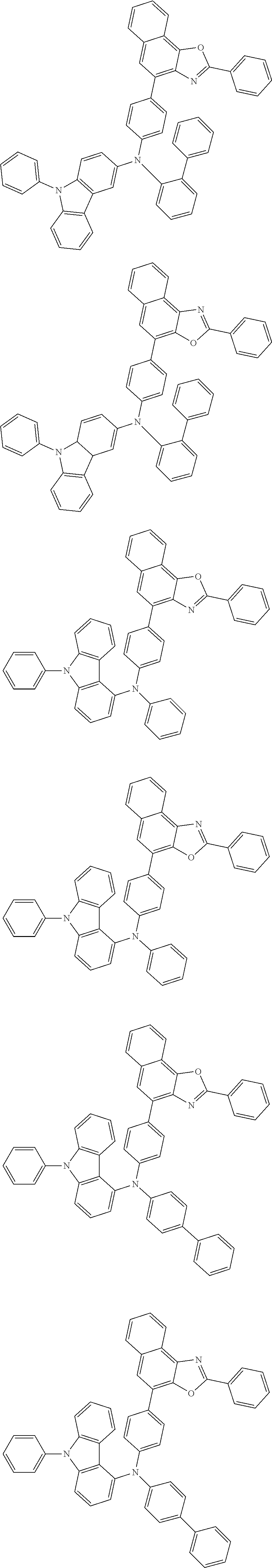

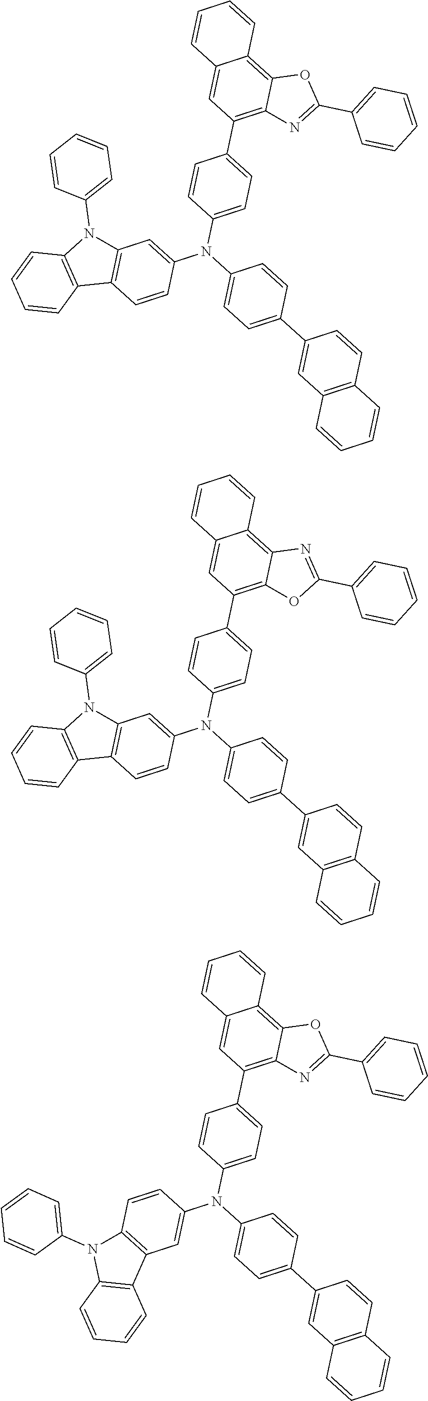 [SMILES: c1ccc(-c2nc3c(-c4ccc(N(c5ccc(-c6ccc7ccccc7c6)cc5)c5ccc6c7ccccc7n(-c7ccccc7)c6c5)cc4)cc4ccccc4c3o2)cc1 ; c1ccc(-c2nc3c(o2)c(-c2ccc(N(c4ccc(-c5ccc6ccccc6c5)cc4)c4ccc5c6ccccc6n(-c6ccccc6)c5c4)cc2)cc2ccccc23)cc1 ; c1ccc(-c2nc3c(-c4ccc(N(c5ccc(-c6ccc7ccccc7c6)cc5)c5ccc6c(c5)c5ccccc5n6-c5ccccc5)cc4)cc4ccccc4c3o2)cc1]

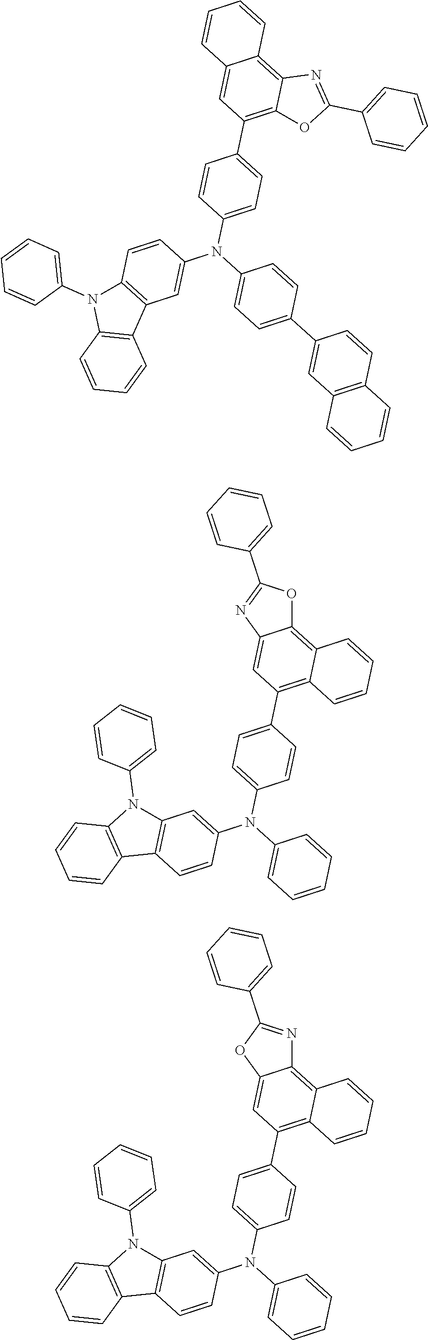 [SMILES: c1ccc(-c2nc3c(o2)c(-c2ccc(N(c4ccc(-c5ccc6ccccc6c5)cc4)c4ccc5c(c4)c4ccccc4n5-c4ccccc4)cc2)cc2ccccc23)cc1 ; c1ccc(-c2nc3cc(-c4ccc(N(c5ccccc5)c5ccc6c7ccccc7n(-c7ccccc7)c6c5)cc4)c4ccccc4c3o2)cc1 ; c1ccc(-c2nc3c(cc(-c4ccc(N(c5ccccc5)c5ccc6c7ccccc7n(-c7ccccc7)c6c5)cc4)c4ccccc43)o2)cc1]

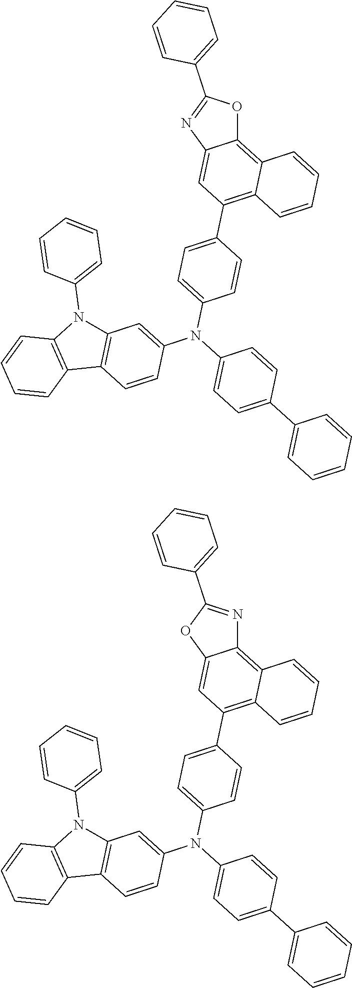 [SMILES: c1ccc(-c2ccc(N(c3ccc(-c4cc5nc(-c6ccccc6)oc5c5ccccc45)cc3)c3ccc4c5ccccc5n(-c5ccccc5)c4c3)cc2)cc1 ; c1ccc(-c2ccc(N(c3ccc(-c4cc5oc(-c6ccccc6)nc5c5ccccc45)cc3)c3ccc4c5ccccc5n(-c5ccccc5)c4c3)cc2)cc1]

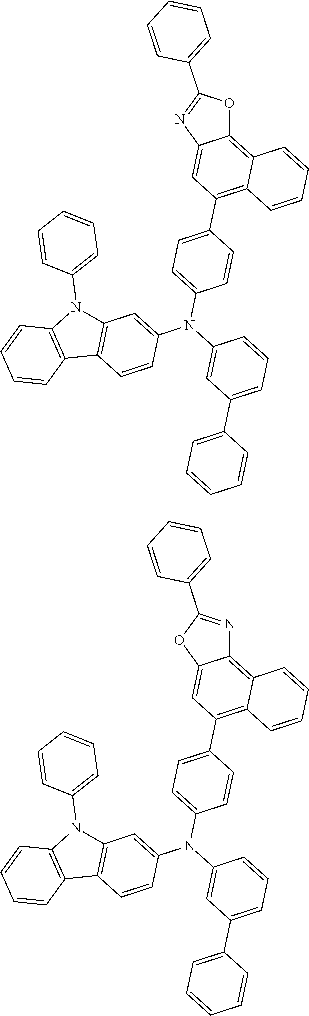

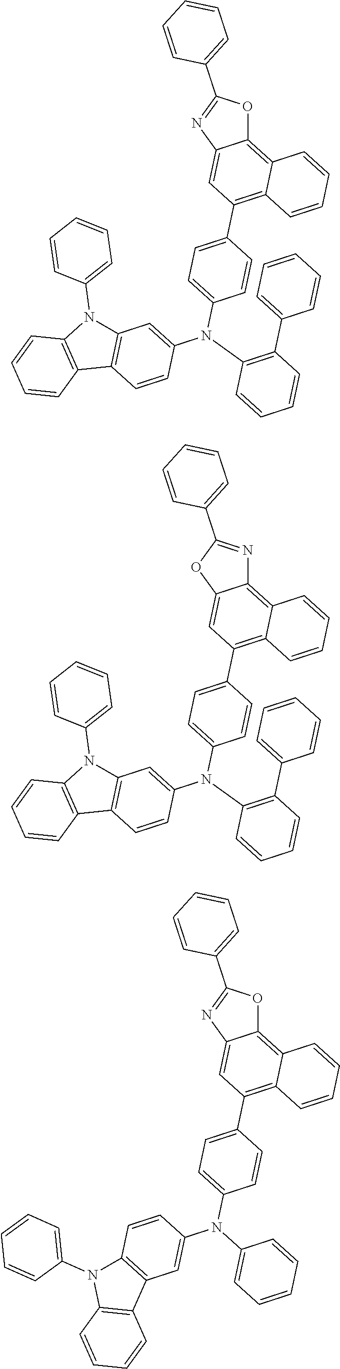 [SMILES: c1ccc(-c2nc3cc(-c4ccc(N(c5ccc6c7ccccc7n(-c7ccccc7)c6c5)c5ccccc5-c5ccccc5)cc4)c4ccccc4c3o2)cc1 ; c1ccc(-c2nc3c(cc(-c4ccc(N(c5ccc6c7ccccc7n(-c7ccccc7)c6c5)c5ccccc5-c5ccccc5)cc4)c4ccccc43)o2)cc1 ; c1ccc(-c2nc3cc(-c4ccc(N(c5ccccc5)c5ccc6c(c5)c5ccccc5n6-c5ccccc5)cc4)c4ccccc4c3o2)cc1]

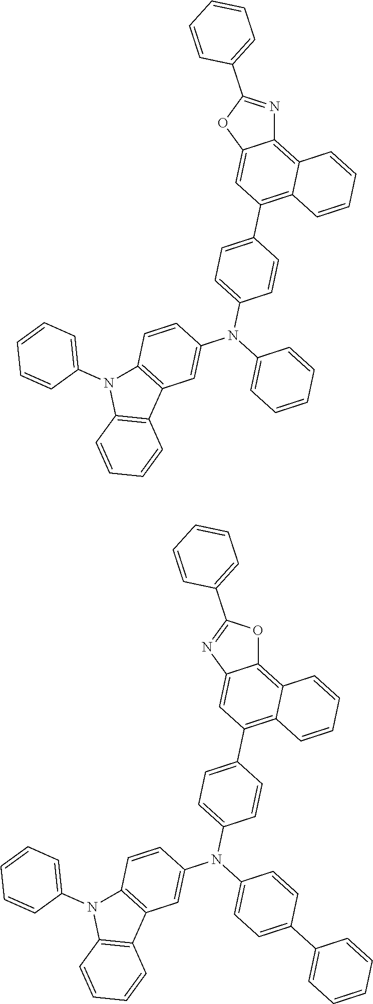 [SMILES: c1ccc(-c2nc3c(cc(-c4ccc(N(c5ccccc5)c5ccc6c(c5)c5ccccc5n6-c5ccccc5)cc4)c4ccccc43)o2)cc1 ; c1ccc(-c2ccc(N(c3ccc(-c4cc5nc(-c6ccccc6)oc5c5ccccc45)cc3)c3ccc4c(c3)c3ccccc3n4-c3ccccc3)cc2)cc1]

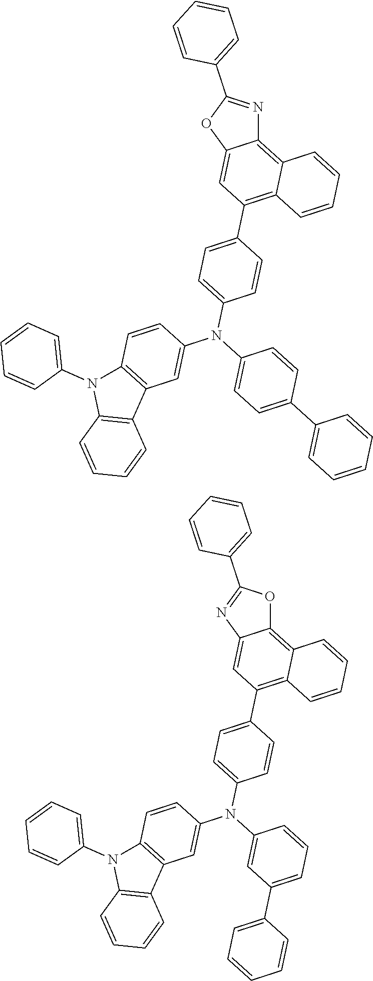 [SMILES: c1ccc(-c2ccc(N(c3ccc(-c4cc5oc(-c6ccccc6)nc5c5ccccc45)cc3)c3ccc4c(c3)c3ccccc3n4-c3ccccc3)cc2)cc1 ; c1ccc(-c2cccc(N(c3ccc(-c4cc5nc(-c6ccccc6)oc5c5ccccc45)cc3)c3ccc4c(c3)c3ccccc3n4-c3ccccc3)c2)cc1]

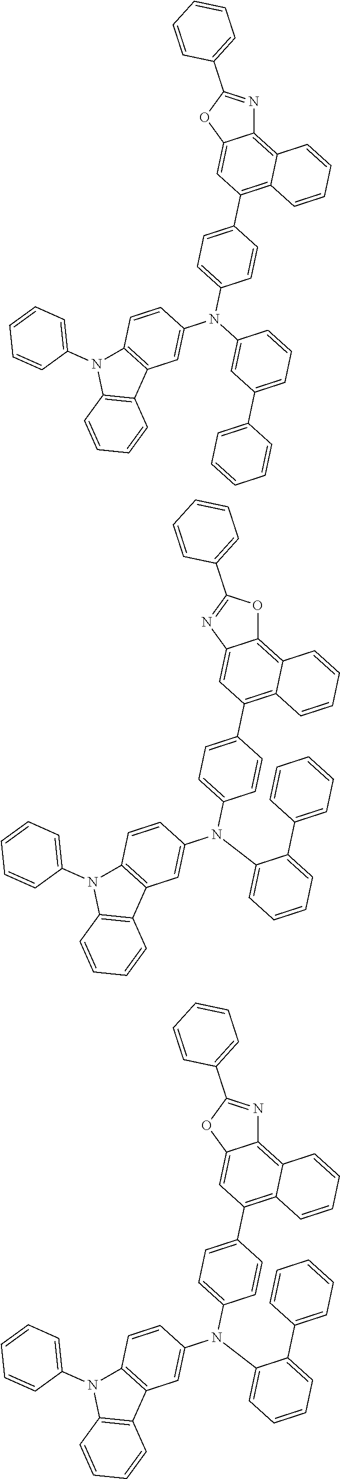

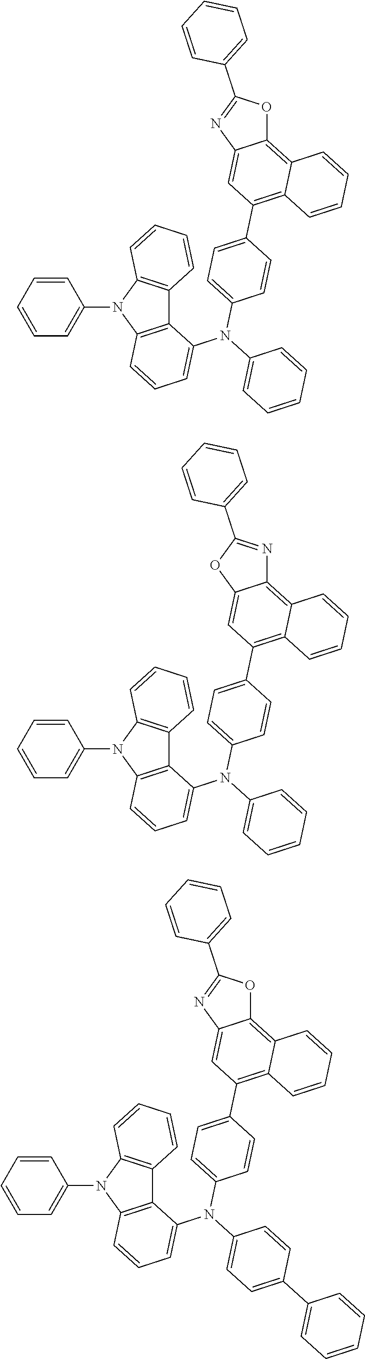 [SMILES: c1ccc(-c2nc3cc(-c4ccc(N(c5ccccc5)c5cccc6c5c5ccccc5n6-c5ccccc5)cc4)c4ccccc4c3o2)cc1 ; c1ccc(-c2nc3c(cc(-c4ccc(N(c5ccccc5)c5cccc6c5c5ccccc5n6-c5ccccc5)cc4)c4ccccc43)o2)cc1 ; c1ccc(-c2ccc(N(c3ccc(-c4cc5nc(-c6ccccc6)oc5c5ccccc45)cc3)c3cccc4c3c3ccccc3n4-c3ccccc3)cc2)cc1]

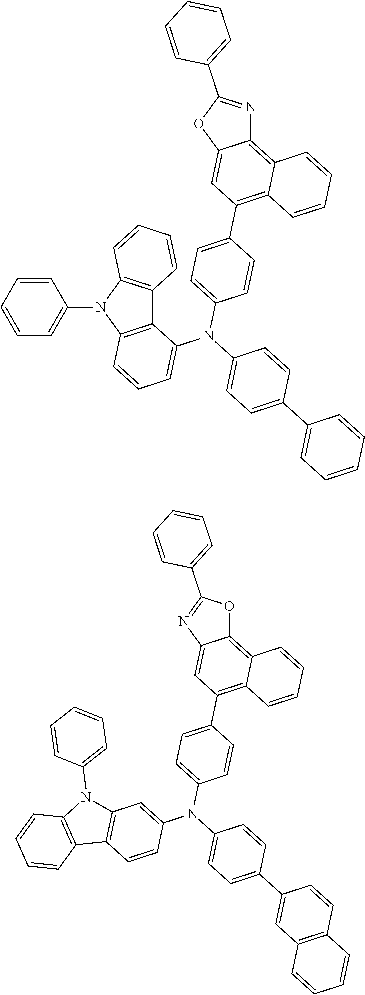 [SMILES: c1ccc(-c2ccc(N(c3ccc(-c4cc5oc(-c6ccccc6)nc5c5ccccc45)cc3)c3cccc4c3c3ccccc3n4-c3ccccc3)cc2)cc1 ; c1ccc(-c2nc3cc(-c4ccc(N(c5ccc(-c6ccc7ccccc7c6)cc5)c5ccc6c7ccccc7n(-c7ccccc7)c6c5)cc4)c4ccccc4c3o2)cc1]

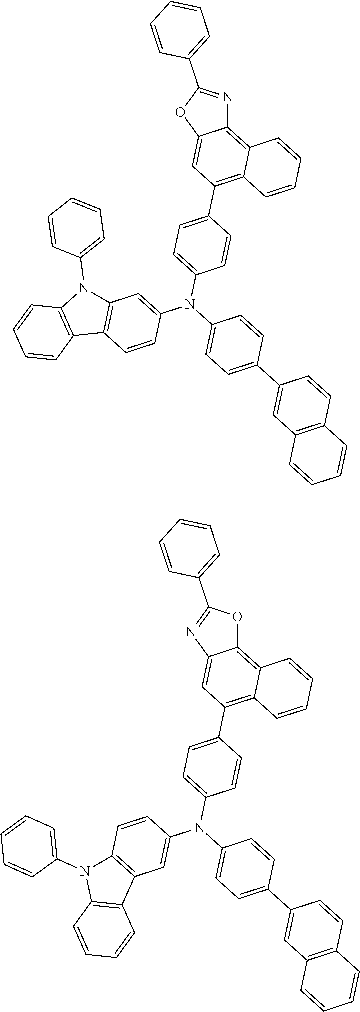 [SMILES: c1ccc(-c2nc3c(cc(-c4ccc(N(c5ccc(-c6ccc7ccccc7c6)cc5)c5ccc6c7ccccc7n(-c7ccccc7)c6c5)cc4)c4ccccc43)o2)cc1 ; c1ccc(-c2nc3cc(-c4ccc(N(c5ccc(-c6ccc7ccccc7c6)cc5)c5ccc6c(c5)c5ccccc5n6-c5ccccc5)cc4)c4ccccc4c3o2)cc1]

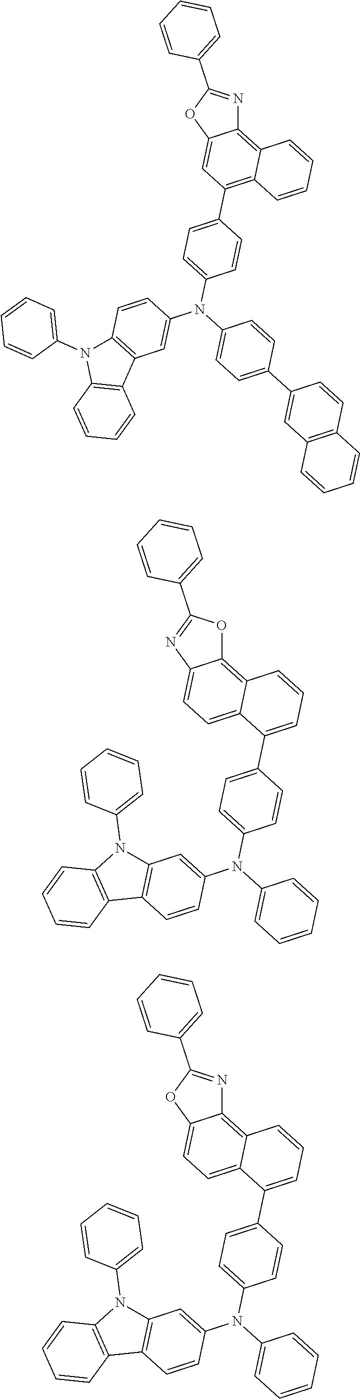 [SMILES: c1ccc(-c2nc3c(cc(-c4ccc(N(c5ccc(-c6ccc7ccccc7c6)cc5)c5ccc6c(c5)c5ccccc5n6-c5ccccc5)cc4)c4ccccc43)o2)cc1 ; c1ccc(-c2nc3ccc4c(-c5ccc(N(c6ccccc6)c6ccc7c8ccccc8n(-c8ccccc8)c7c6)cc5)cccc4c3o2)cc1 ; c1ccc(-c2nc3c(ccc4c(-c5ccc(N(c6ccccc6)c6ccc7c8ccccc8n(-c8ccccc8)c7c6)cc5)cccc43)o2)cc1]

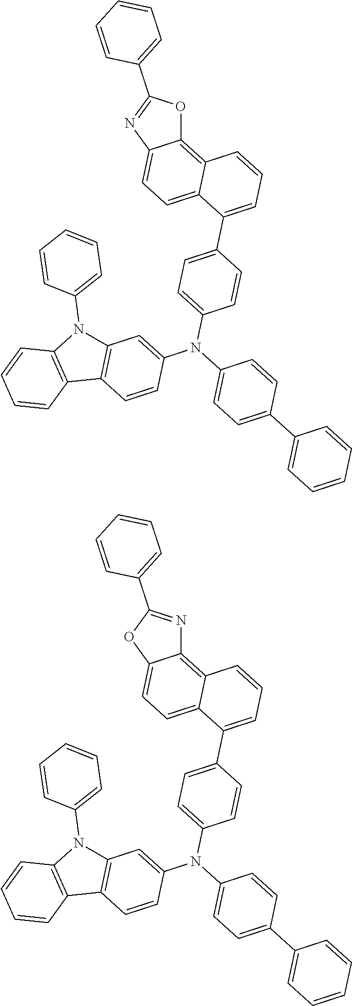 [SMILES: c1ccc(-c2ccc(N(c3ccc(-c4cccc5c4ccc4nc(-c6ccccc6)oc45)cc3)c3ccc4c5ccccc5n(-c5ccccc5)c4c3)cc2)cc1 ; c1ccc(-c2ccc(N(c3ccc(-c4cccc5c4ccc4oc(-c6ccccc6)nc45)cc3)c3ccc4c5ccccc5n(-c5ccccc5)c4c3)cc2)cc1]

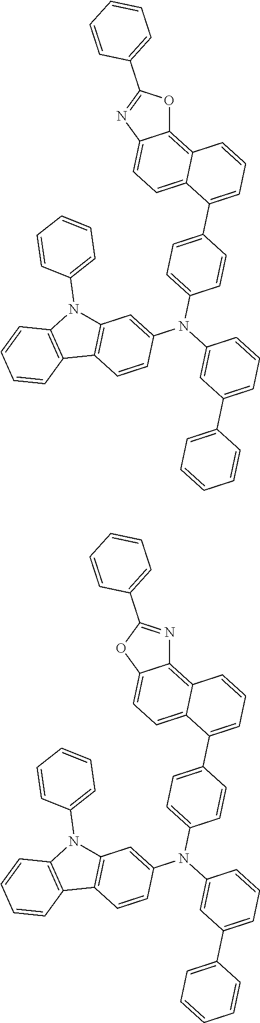 [SMILES: c1ccc(-c2cccc(N(c3ccc(-c4cccc5c4ccc4nc(-c6ccccc6)oc45)cc3)c3ccc4c5ccccc5n(-c5ccccc5)c4c3)c2)cc1 ; c1ccc(-c2cccc(N(c3ccc(-c4cccc5c4ccc4oc(-c6ccccc6)nc45)cc3)c3ccc4c5ccccc5n(-c5ccccc5)c4c3)c2)cc1]

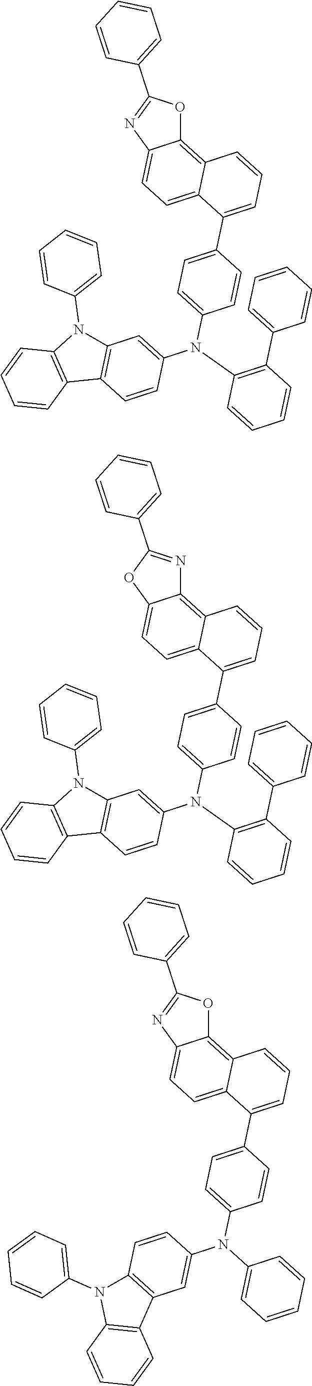 [SMILES: c1ccc(-c2nc3ccc4c(-c5ccc(N(c6ccc7c8ccccc8n(-c8ccccc8)c7c6)c6ccccc6-c6ccccc6)cc5)cccc4c3o2)cc1 ; c1ccc(-c2nc3c(ccc4c(-c5ccc(N(c6ccc7c8ccccc8n(-c8ccccc8)c7c6)c6ccccc6-c6ccccc6)cc5)cccc43)o2)cc1 ; c1ccc(-c2nc3ccc4c(-c5ccc(N(c6ccccc6)c6ccc7c(c6)c6ccccc6n7-c6ccccc6)cc5)cccc4c3o2)cc1]

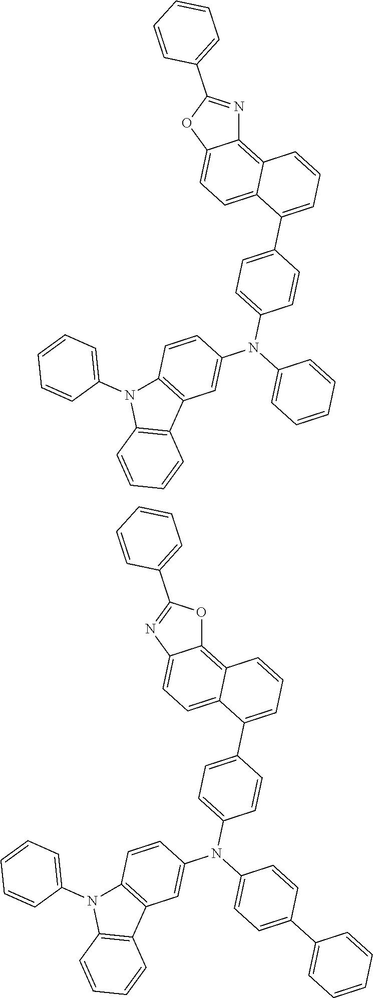

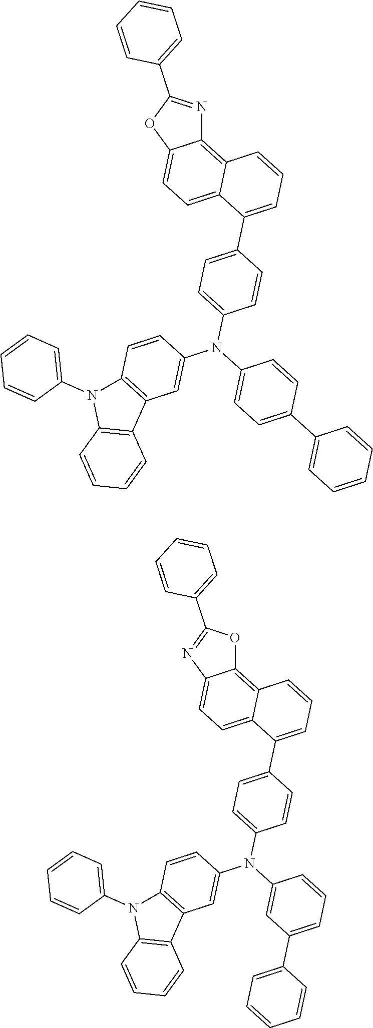 [SMILES: c1ccc(-c2ccc(N(c3ccc(-c4cccc5c4ccc4oc(-c6ccccc6)nc45)cc3)c3ccc4c(c3)c3ccccc3n4-c3ccccc3)cc2)cc1 ; c1ccc(-c2cccc(N(c3ccc(-c4cccc5c4ccc4nc(-c6ccccc6)oc45)cc3)c3ccc4c(c3)c3ccccc3n4-c3ccccc3)c2)cc1]

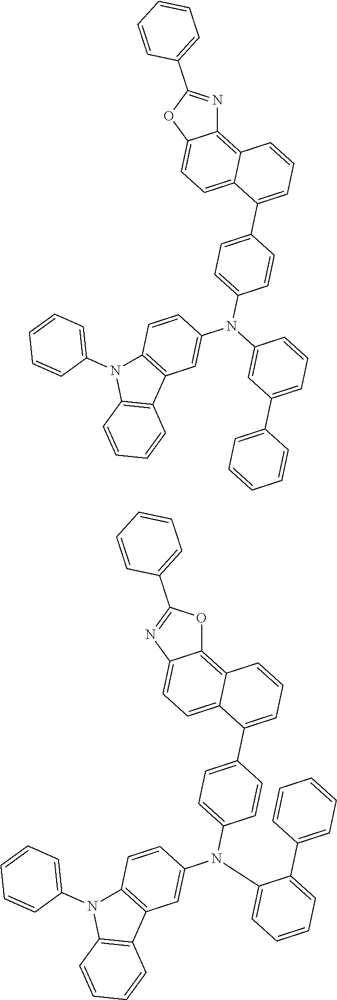 [SMILES: c1ccc(-c2cccc(N(c3ccc(-c4cccc5c4ccc4oc(-c6ccccc6)nc45)cc3)c3ccc4c(c3)c3ccccc3n4-c3ccccc3)c2)cc1 ; c1ccc(-c2nc3ccc4c(-c5ccc(N(c6ccc7c(c6)c6ccccc6n7-c6ccccc6)c6ccccc6-c6ccccc6)cc5)cccc4c3o2)cc1]

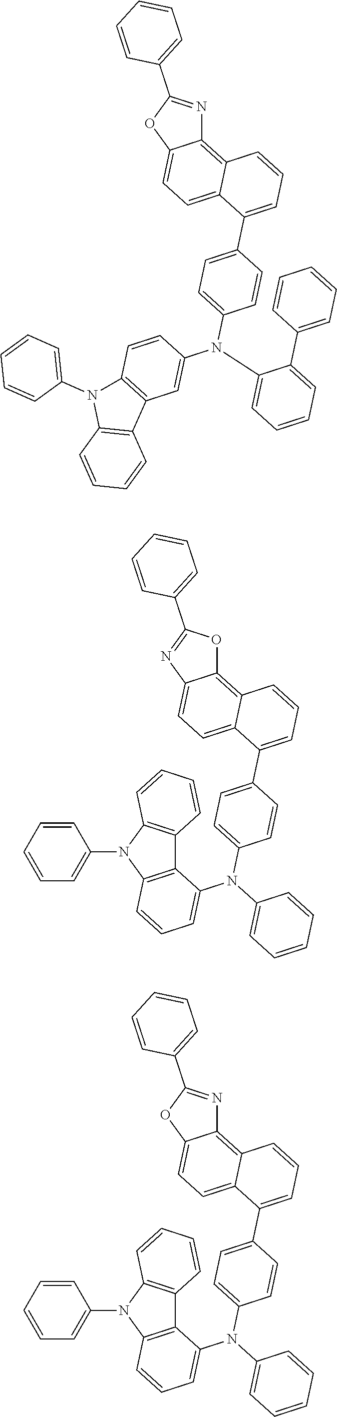 [SMILES: c1ccc(-c2nc3c(ccc4c(-c5ccc(N(c6ccc7c(c6)c6ccccc6n7-c6ccccc6)c6ccccc6-c6ccccc6)cc5)cccc43)o2)cc1 ; c1ccc(-c2nc3ccc4c(-c5ccc(N(c6ccccc6)c6cccc7c6c6ccccc6n7-c6ccccc6)cc5)cccc4c3o2)cc1 ; c1ccc(-c2nc3c(ccc4c(-c5ccc(N(c6ccccc6)c6cccc7c6c6ccccc6n7-c6ccccc6)cc5)cccc43)o2)cc1]

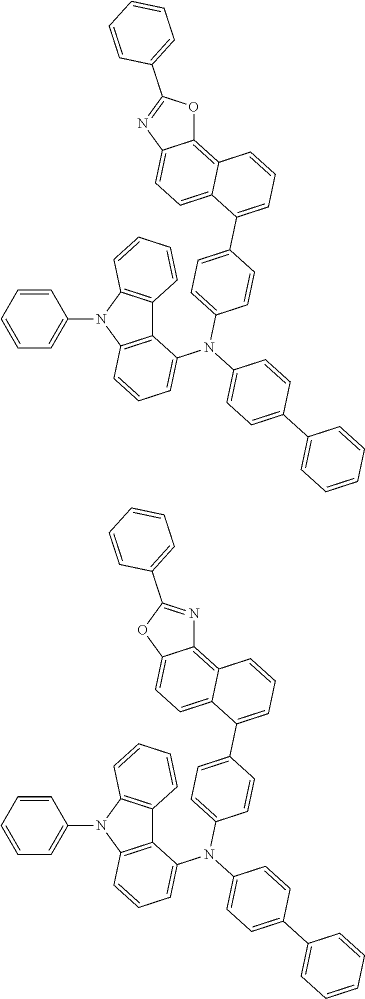 [SMILES: c1ccc(-c2ccc(N(c3ccc(-c4cccc5c4ccc4nc(-c6ccccc6)oc45)cc3)c3cccc4c3c3ccccc3n4-c3ccccc3)cc2)cc1 ; c1ccc(-c2ccc(N(c3ccc(-c4cccc5c4ccc4oc(-c6ccccc6)nc45)cc3)c3cccc4c3c3ccccc3n4-c3ccccc3)cc2)cc1]

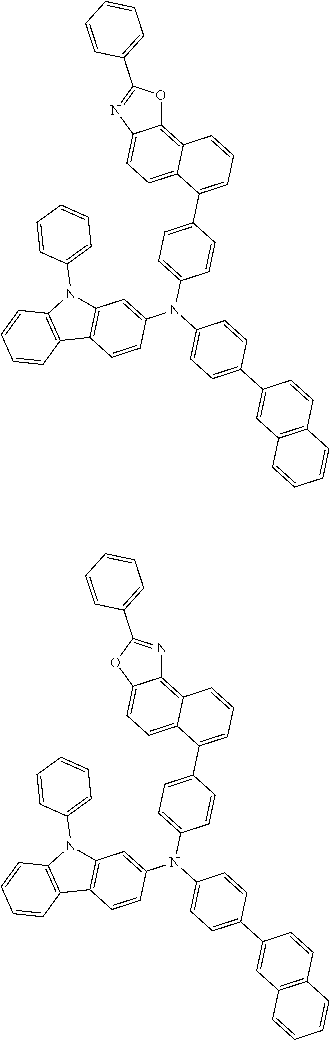 [SMILES: c1ccc(-c2nc3ccc4c(-c5ccc(N(c6ccc(-c7ccc8ccccc8c7)cc6)c6ccc7c8ccccc8n(-c8ccccc8)c7c6)cc5)cccc4c3o2)cc1 ; c1ccc(-c2nc3c(ccc4c(-c5ccc(N(c6ccc(-c7ccc8ccccc8c7)cc6)c6ccc7c8ccccc8n(-c8ccccc8)c7c6)cc5)cccc43)o2)cc1]

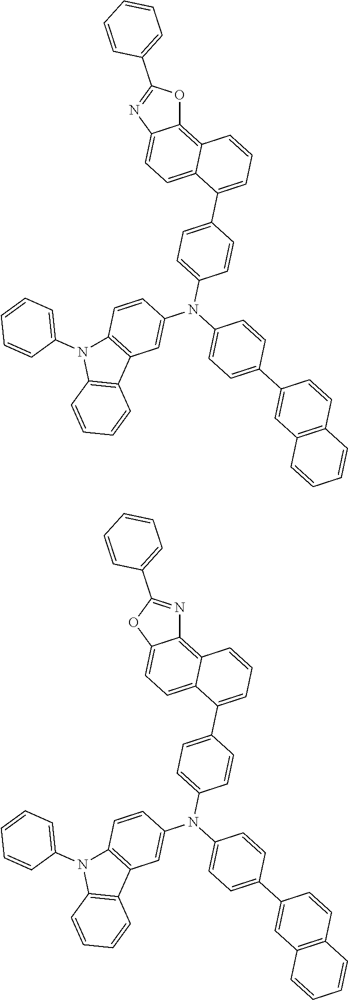 [SMILES: c1ccc(-c2nc3ccc4c(-c5ccc(N(c6ccc(-c7ccc8ccccc8c7)cc6)c6ccc7c(c6)c6ccccc6n7-c6ccccc6)cc5)cccc4c3o2)cc1 ; c1ccc(-c2nc3c(ccc4c(-c5ccc(N(c6ccc(-c7ccc8ccccc8c7)cc6)c6ccc7c(c6)c6ccccc6n7-c6ccccc6)cc5)cccc43)o2)cc1]

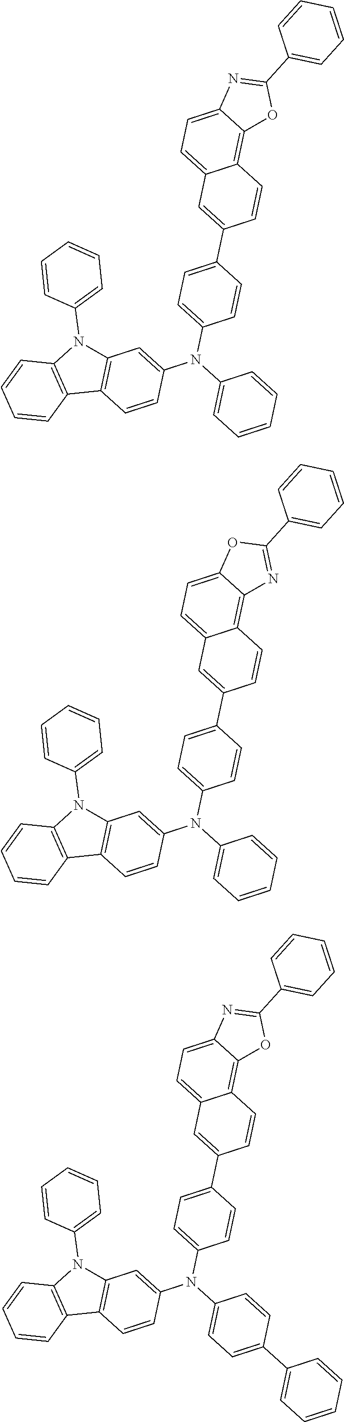 [SMILES: c1ccc(-c2nc3ccc4cc(-c5ccc(N(c6ccccc6)c6ccc7c8ccccc8n(-c8ccccc8)c7c6)cc5)ccc4c3o2)cc1 ; c1ccc(-c2nc3c(ccc4cc(-c5ccc(N(c6ccccc6)c6ccc7c8ccccc8n(-c8ccccc8)c7c6)cc5)ccc43)o2)cc1 ; c1ccc(-c2ccc(N(c3ccc(-c4ccc5c(ccc6nc(-c7ccccc7)oc65)c4)cc3)c3ccc4c5ccccc5n(-c5ccccc5)c4c3)cc2)cc1]

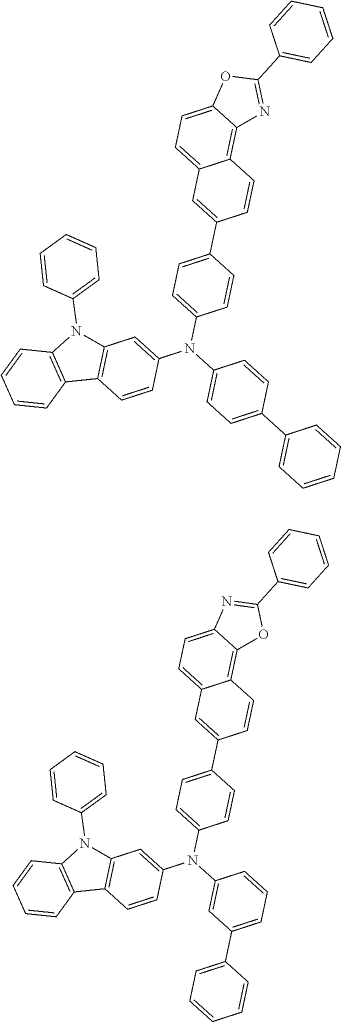

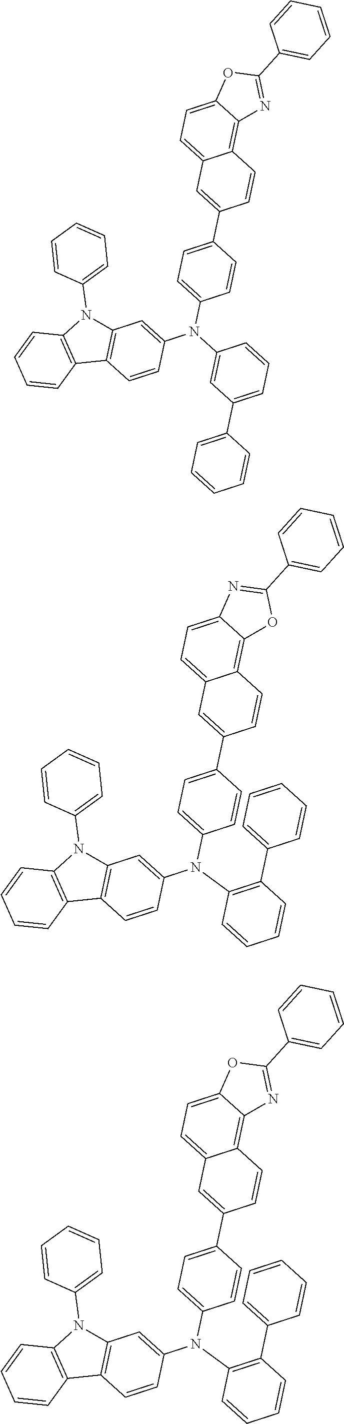 [SMILES: c1ccc(-c2cccc(N(c3ccc(-c4ccc5c(ccc6oc(-c7ccccc7)nc65)c4)cc3)c3ccc4c5ccccc5n(-c5ccccc5)c4c3)c2)cc1 ; c1ccc(-c2nc3ccc4cc(-c5ccc(N(c6ccc7c8ccccc8n(-c8ccccc8)c7c6)c6ccccc6-c6ccccc6)cc5)ccc4c3o2)cc1 ; c1ccc(-c2nc3c(ccc4cc(-c5ccc(N(c6ccc7c8ccccc8n(-c8ccccc8)c7c6)c6ccccc6-c6ccccc6)cc5)ccc43)o2)cc1]

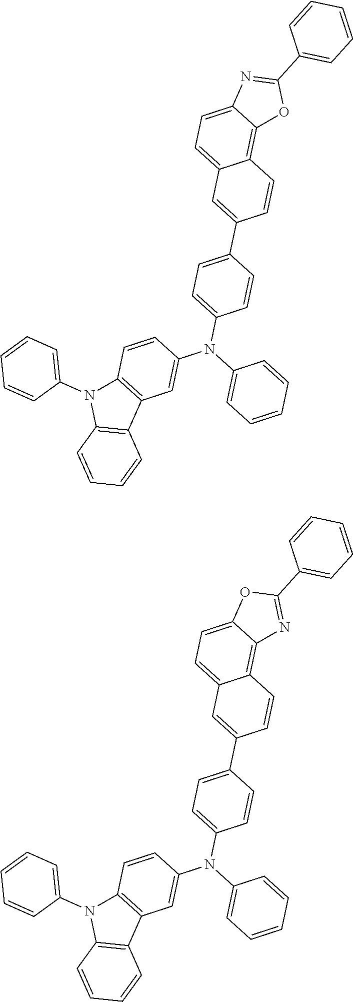 [SMILES: c1ccc(-c2nc3ccc4cc(-c5ccc(N(c6ccccc6)c6ccc7c(c6)c6ccccc6n7-c6ccccc6)cc5)ccc4c3o2)cc1 ; c1ccc(-c2nc3c(ccc4cc(-c5ccc(N(c6ccccc6)c6ccc7c(c6)c6ccccc6n7-c6ccccc6)cc5)ccc43)o2)cc1]

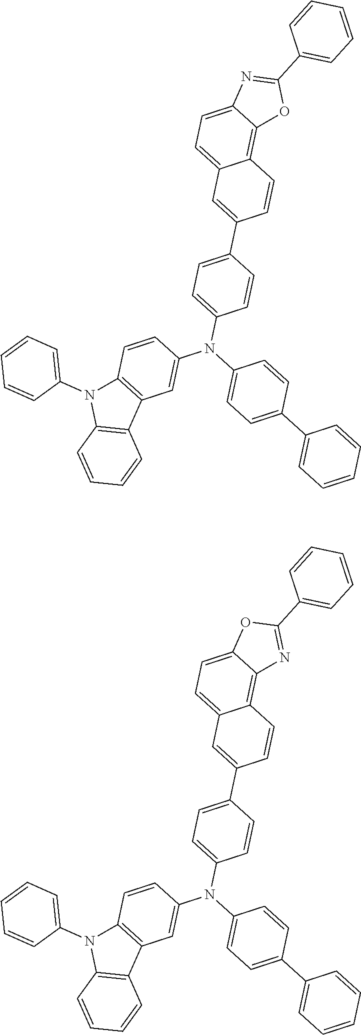 [SMILES: c1ccc(-c2ccc(N(c3ccc(-c4ccc5c(ccc6nc(-c7ccccc7)oc65)c4)cc3)c3ccc4c(c3)c3ccccc3n4-c3ccccc3)cc2)cc1 ; c1ccc(-c2ccc(N(c3ccc(-c4ccc5c(ccc6oc(-c7ccccc7)nc65)c4)cc3)c3ccc4c(c3)c3ccccc3n4-c3ccccc3)cc2)cc1]

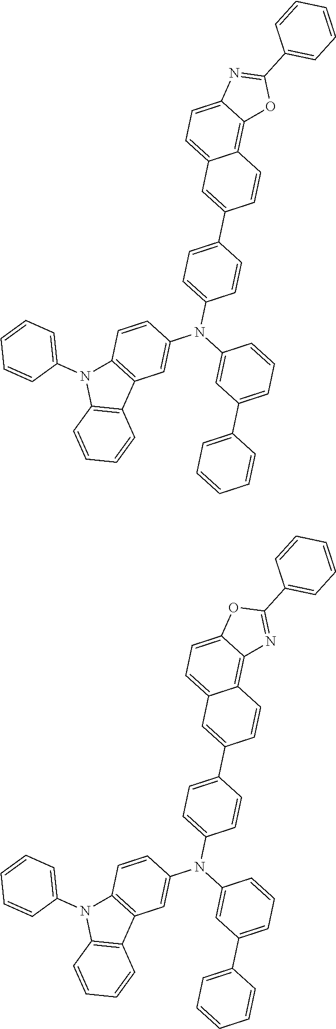 [SMILES: c1ccc(-c2cccc(N(c3ccc(-c4ccc5c(ccc6nc(-c7ccccc7)oc65)c4)cc3)c3ccc4c(c3)c3ccccc3n4-c3ccccc3)c2)cc1 ; c1ccc(-c2cccc(N(c3ccc(-c4ccc5c(ccc6oc(-c7ccccc7)nc65)c4)cc3)c3ccc4c(c3)c3ccccc3n4-c3ccccc3)c2)cc1]

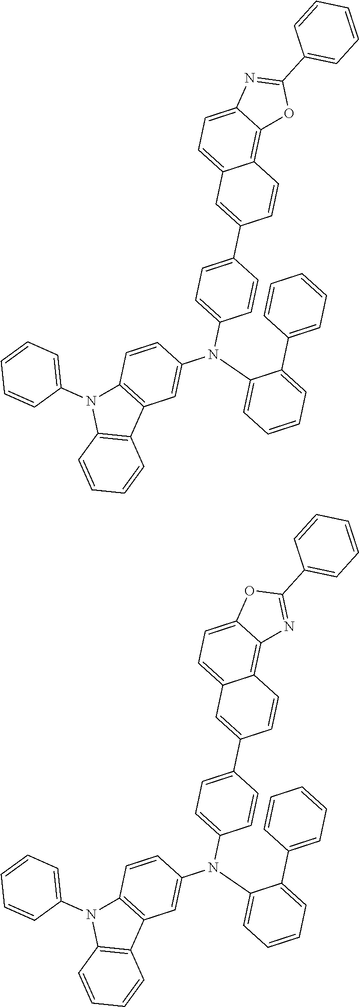 [SMILES: c1ccc(-c2nc3ccc4cc(-c5ccc(N(c6ccc7c(c6)c6ccccc6n7-c6ccccc6)c6ccccc6-c6ccccc6)cc5)ccc4c3o2)cc1 ; c1ccc(-c2nc3c(ccc4cc(-c5ccc(N(c6ccc7c(c6)c6ccccc6n7-c6ccccc6)c6ccccc6-c6ccccc6)cc5)ccc43)o2)cc1]

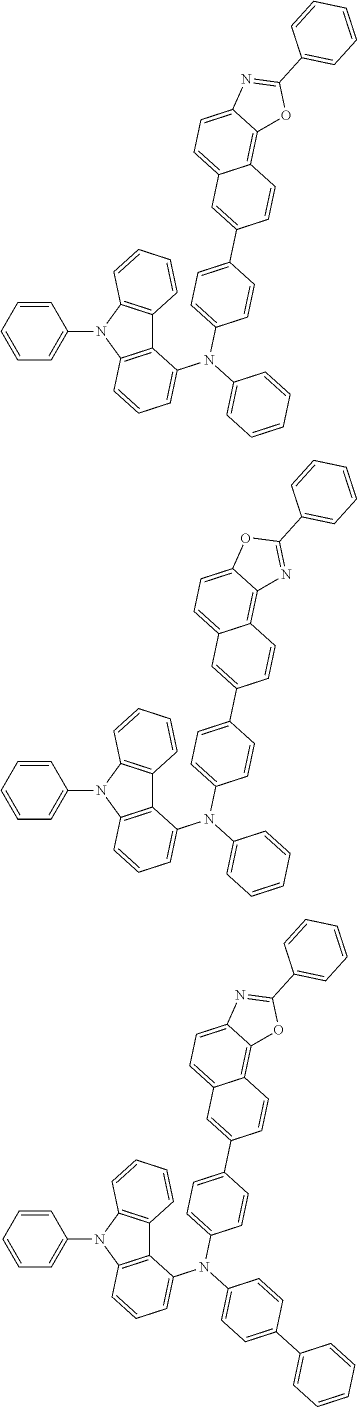

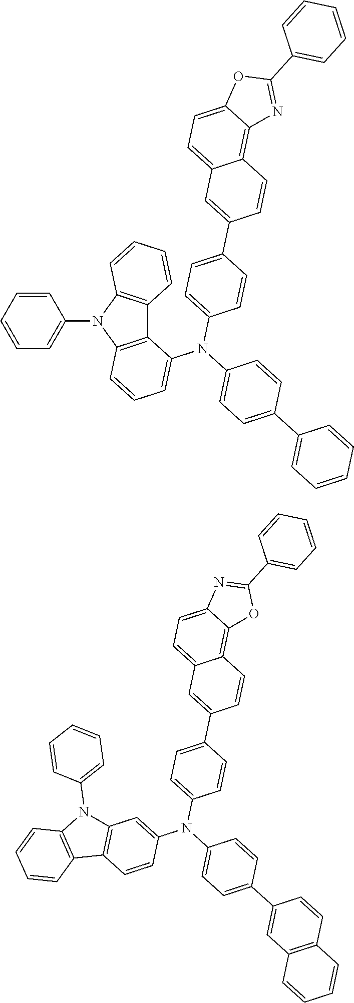

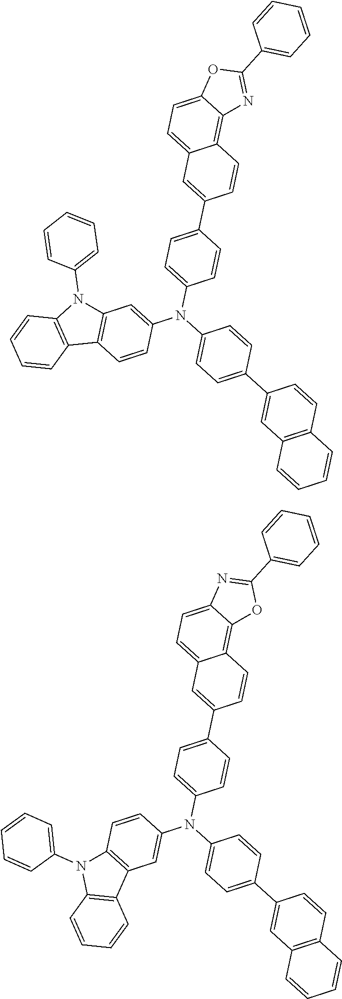

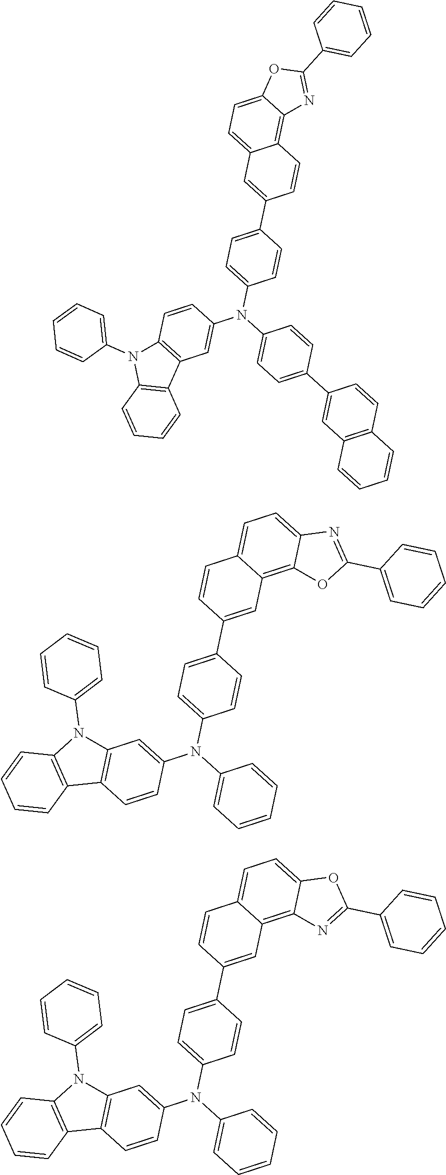

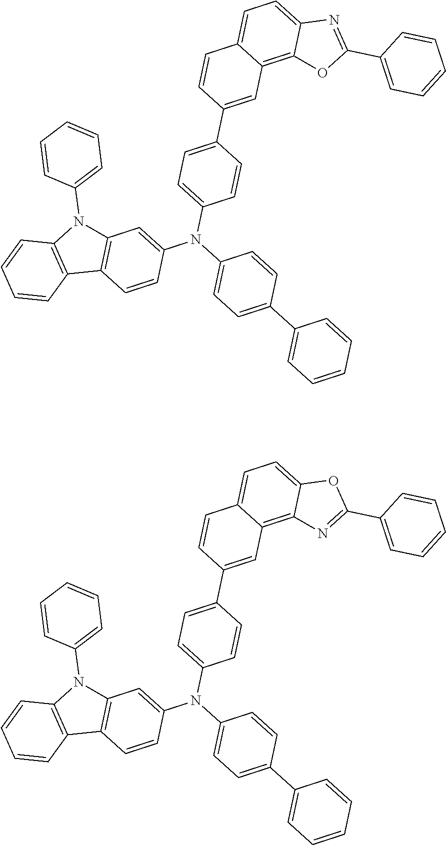 [SMILES: c1ccc(-c2ccc(N(c3ccc(-c4ccc5ccc6nc(-c7ccccc7)oc6c5c4)cc3)c3ccc4c5ccccc5n(-c5ccccc5)c4c3)cc2)cc1 ; c1ccc(-c2ccc(N(c3ccc(-c4ccc5ccc6oc(-c7ccccc7)nc6c5c4)cc3)c3ccc4c5ccccc5n(-c5ccccc5)c4c3)cc2)cc1]

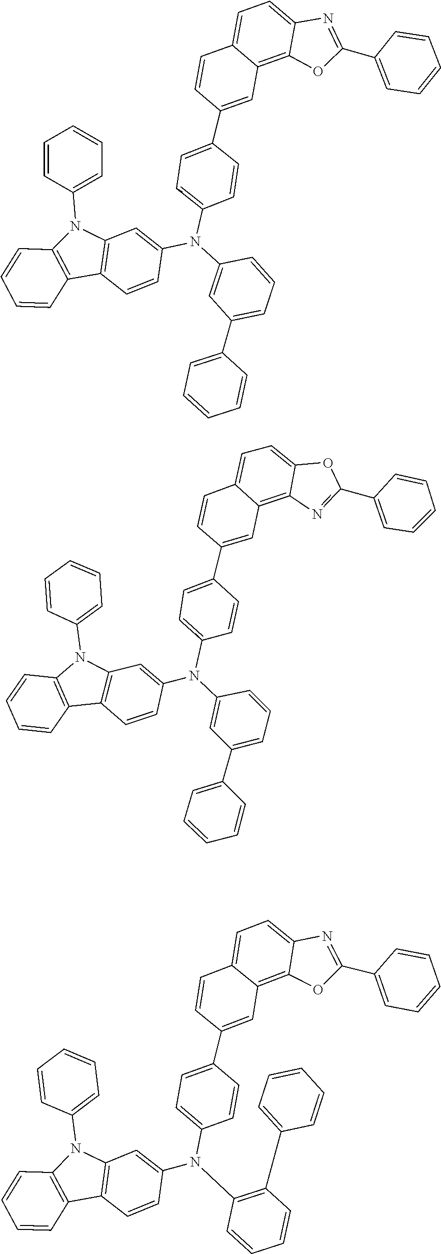

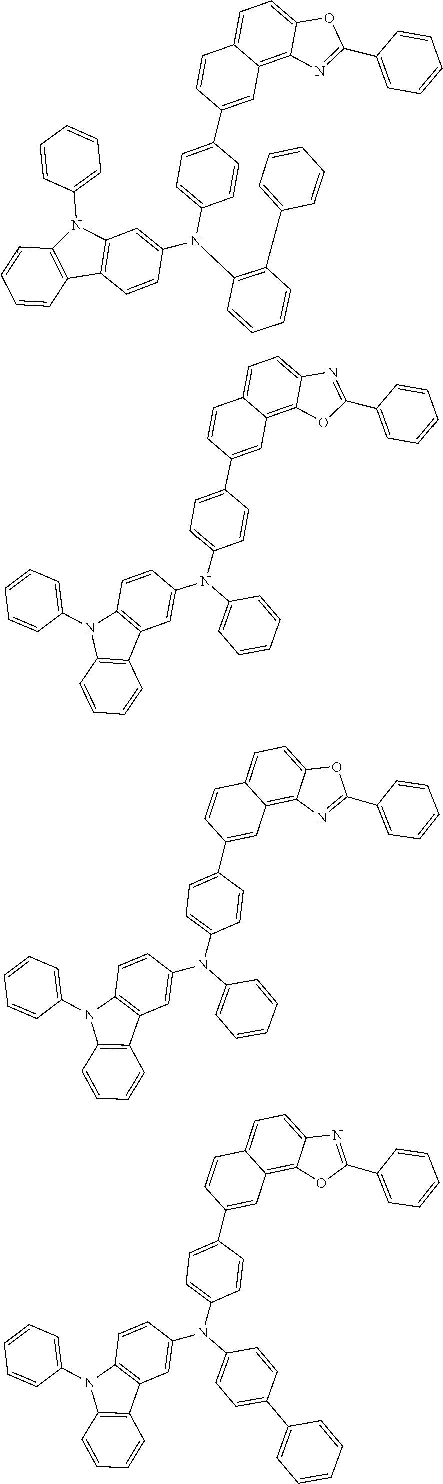

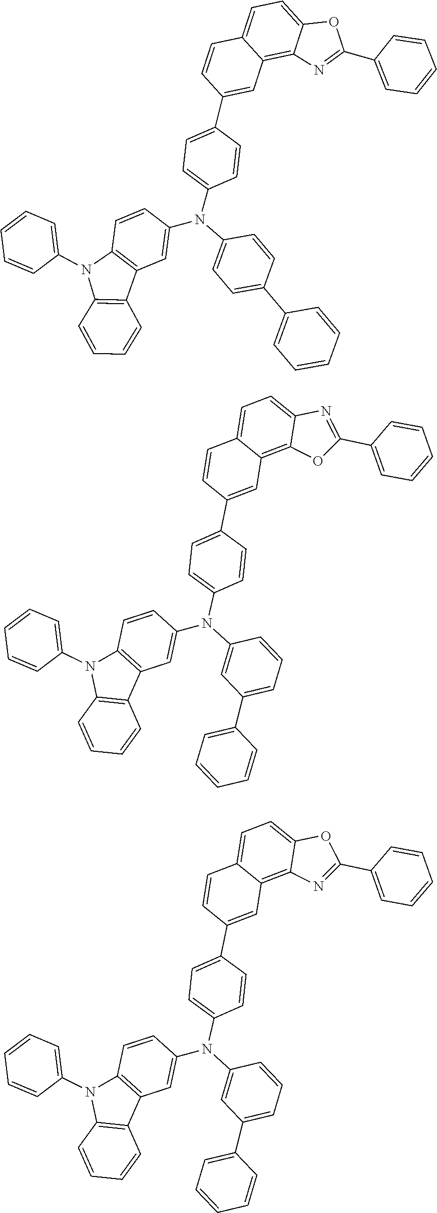

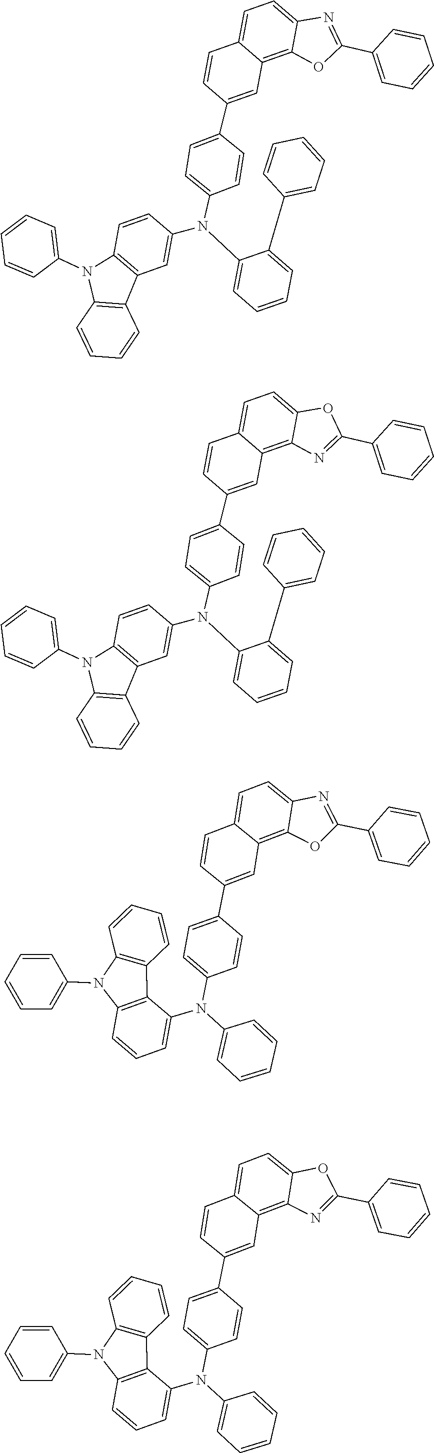 [SMILES: c1ccc(-c2nc3ccc4ccc(-c5ccc(N(c6ccc7c(c6)c6ccccc6n7-c6ccccc6)c6ccccc6-c6ccccc6)cc5)cc4c3o2)cc1 ; c1ccc(-c2nc3c(ccc4ccc(-c5ccc(N(c6ccc7c(c6)c6ccccc6n7-c6ccccc6)c6ccccc6-c6ccccc6)cc5)cc43)o2)cc1 ; c1ccc(-c2nc3ccc4ccc(-c5ccc(N(c6ccccc6)c6cccc7c6c6ccccc6n7-c6ccccc6)cc5)cc4c3o2)cc1 ; c1ccc(-c2nc3c(ccc4ccc(-c5ccc(N(c6ccccc6)c6cccc7c6c6ccccc6n7-c6ccccc6)cc5)cc43)o2)cc1]

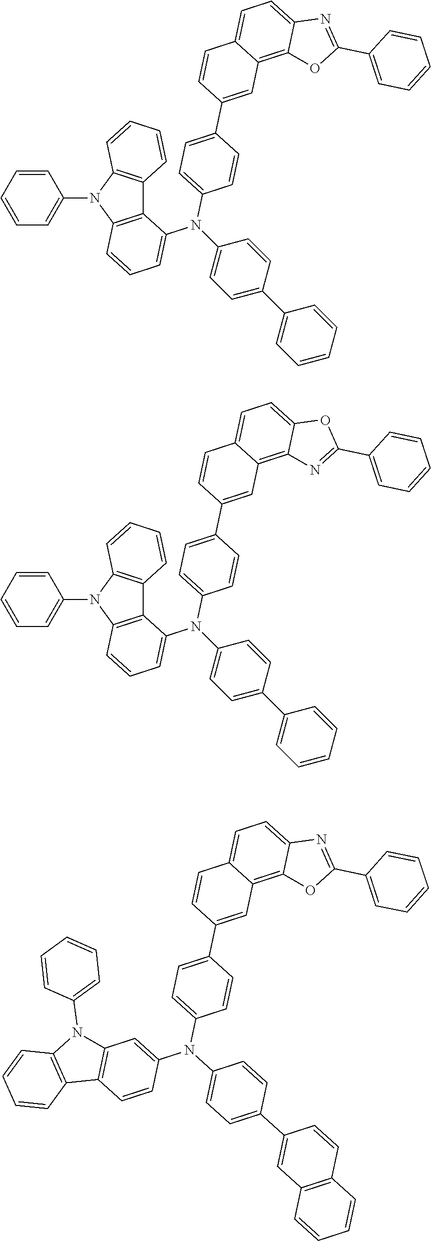 [SMILES: c1ccc(-c2ccc(N(c3ccc(-c4ccc5ccc6nc(-c7ccccc7)oc6c5c4)cc3)c3cccc4c3c3ccccc3n4-c3ccccc3)cc2)cc1 ; c1ccc(-c2ccc(N(c3ccc(-c4ccc5ccc6oc(-c7ccccc7)nc6c5c4)cc3)c3cccc4c3c3ccccc3n4-c3ccccc3)cc2)cc1 ; c1ccc(-c2nc3ccc4ccc(-c5ccc(N(c6ccc(-c7ccc8ccccc8c7)cc6)c6ccc7c8ccccc8n(-c8ccccc8)c7c6)cc5)cc4c3o2)cc1]

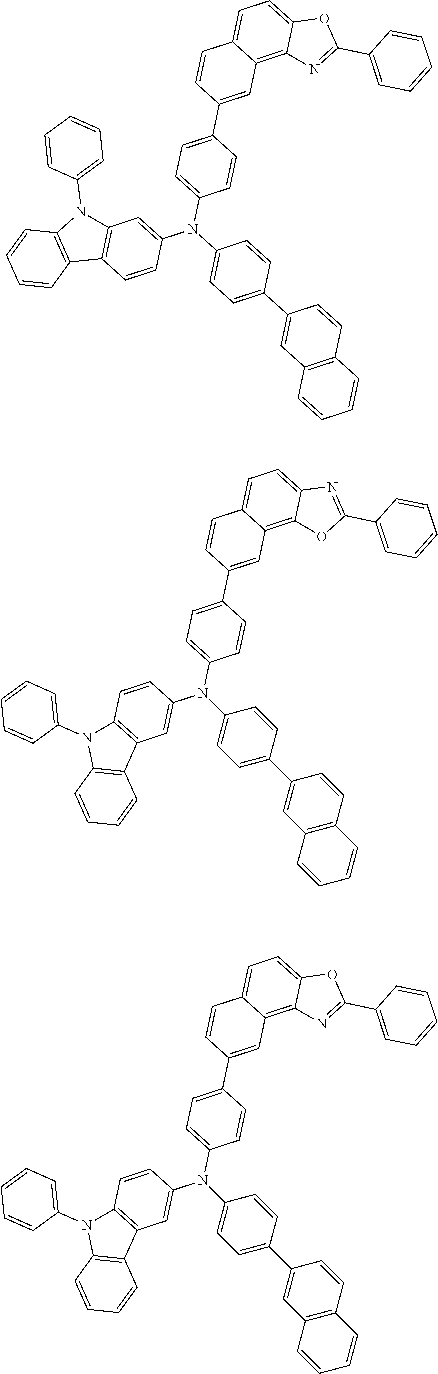 [SMILES: c1ccc(-c2nc3c(ccc4ccc(-c5ccc(N(c6ccc(-c7ccc8ccccc8c7)cc6)c6ccc7c8ccccc8n(-c8ccccc8)c7c6)cc5)cc43)o2)cc1 ; c1ccc(-c2nc3ccc4ccc(-c5ccc(N(c6ccc(-c7ccc8ccccc8c7)cc6)c6ccc7c(c6)c6ccccc6n7-c6ccccc6)cc5)cc4c3o2)cc1 ; c1ccc(-c2nc3c(ccc4ccc(-c5ccc(N(c6ccc(-c7ccc8ccccc8c7)cc6)c6ccc7c(c6)c6ccccc6n7-c6ccccc6)cc5)cc43)o2)cc1]

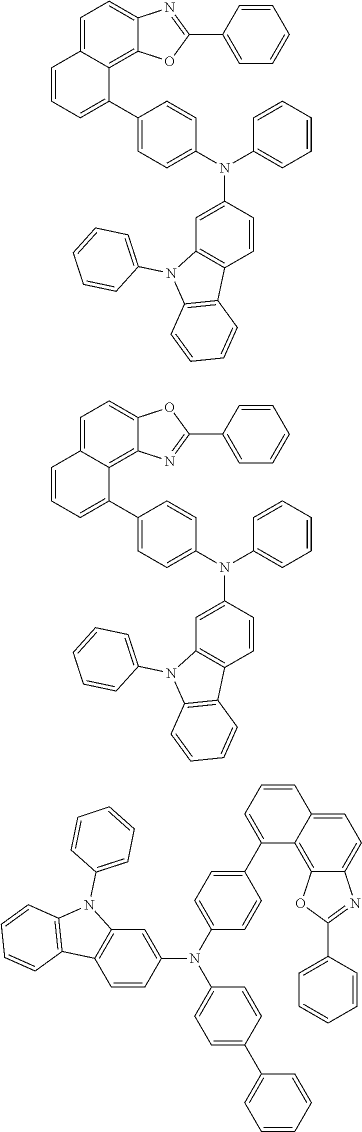 [SMILES: c1ccc(-c2nc3ccc4cccc(-c5ccc(N(c6ccccc6)c6ccc7c8ccccc8n(-c8ccccc8)c7c6)cc5)c4c3o2)cc1 ; c1ccc(-c2nc3c(ccc4cccc(-c5ccc(N(c6ccccc6)c6ccc7c8ccccc8n(-c8ccccc8)c7c6)cc5)c43)o2)cc1 ; c1ccc(-c2ccc(N(c3ccc(-c4cccc5ccc6nc(-c7ccccc7)oc6c45)cc3)c3ccc4c5ccccc5n(-c5ccccc5)c4c3)cc2)cc1]

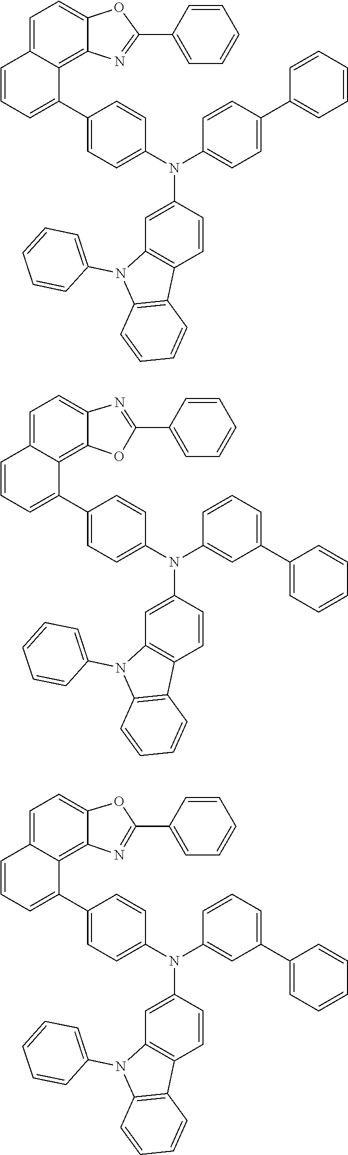

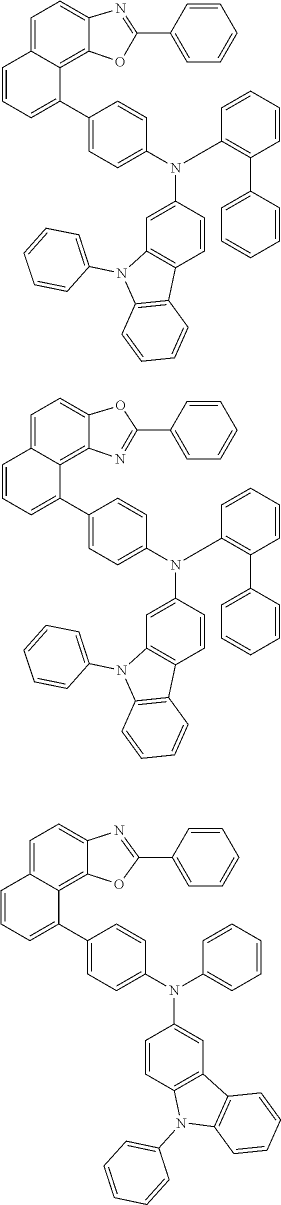

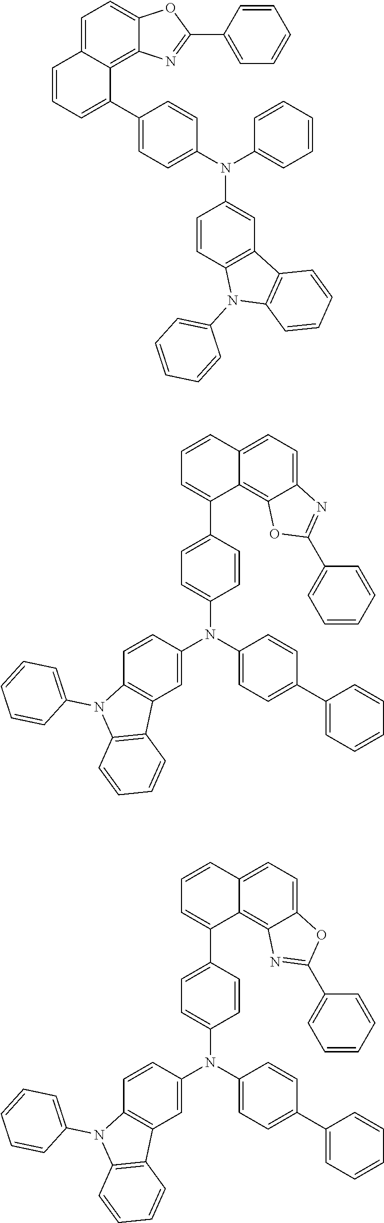 [SMILES: c1ccc(-c2nc3c(ccc4cccc(-c5ccc(N(c6ccccc6)c6ccc7c(c6)c6ccccc6n7-c6ccccc6)cc5)c43)o2)cc1 ; c1ccc(-c2ccc(N(c3ccc(-c4cccc5ccc6nc(-c7ccccc7)oc6c45)cc3)c3ccc4c(c3)c3ccccc3n4-c3ccccc3)cc2)cc1 ; c1ccc(-c2ccc(N(c3ccc(-c4cccc5ccc6oc(-c7ccccc7)nc6c45)cc3)c3ccc4c(c3)c3ccccc3n4-c3ccccc3)cc2)cc1]

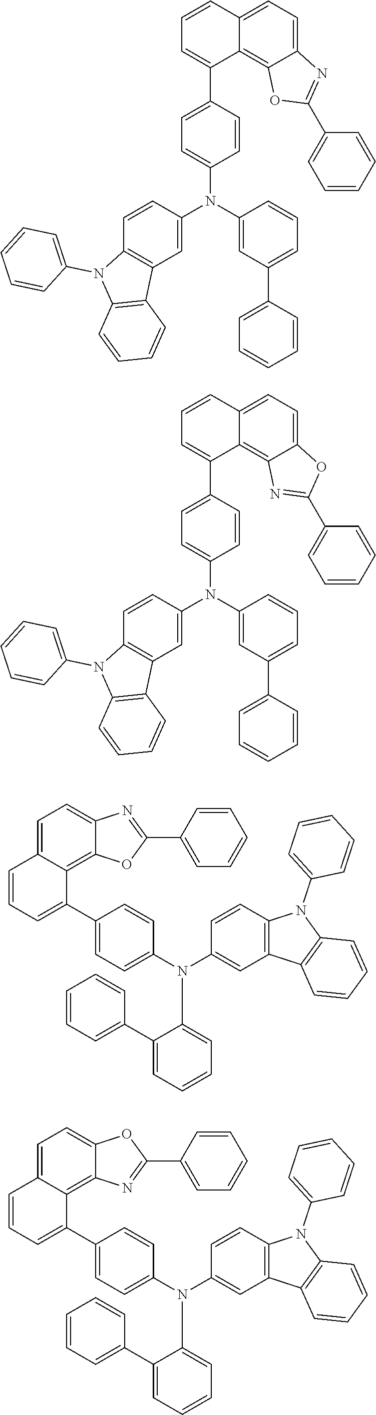

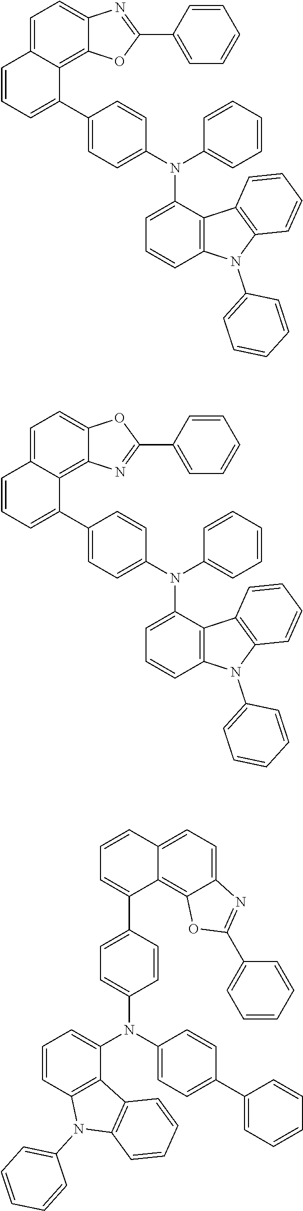 [SMILES: c1ccc(-c2nc3ccc4cccc(-c5ccc(N(c6ccccc6)c6cccc7c6c6ccccc6n7-c6ccccc6)cc5)c4c3o2)cc1 ; c1ccc(-c2nc3c(ccc4cccc(-c5ccc(N(c6ccccc6)c6cccc7c6c6ccccc6n7-c6ccccc6)cc5)c43)o2)cc1 ; c1ccc(-c2ccc(N(c3ccc(-c4cccc5ccc6nc(-c7ccccc7)oc6c45)cc3)c3cccc4c3c3ccccc3n4-c3ccccc3)cc2)cc1]

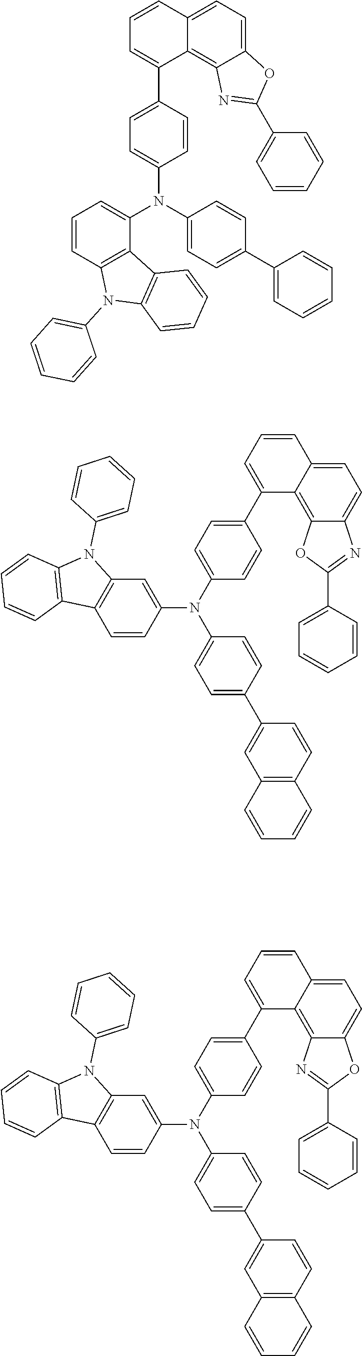 [SMILES: c1ccc(-c2ccc(N(c3ccc(-c4cccc5ccc6oc(-c7ccccc7)nc6c45)cc3)c3cccc4c3c3ccccc3n4-c3ccccc3)cc2)cc1 ; c1ccc(-c2nc3ccc4cccc(-c5ccc(N(c6ccc(-c7ccc8ccccc8c7)cc6)c6ccc7c8ccccc8n(-c8ccccc8)c7c6)cc5)c4c3o2)cc1 ; c1ccc(-c2nc3c(ccc4cccc(-c5ccc(N(c6ccc(-c7ccc8ccccc8c7)cc6)c6ccc7c8ccccc8n(-c8ccccc8)c7c6)cc5)c43)o2)cc1]

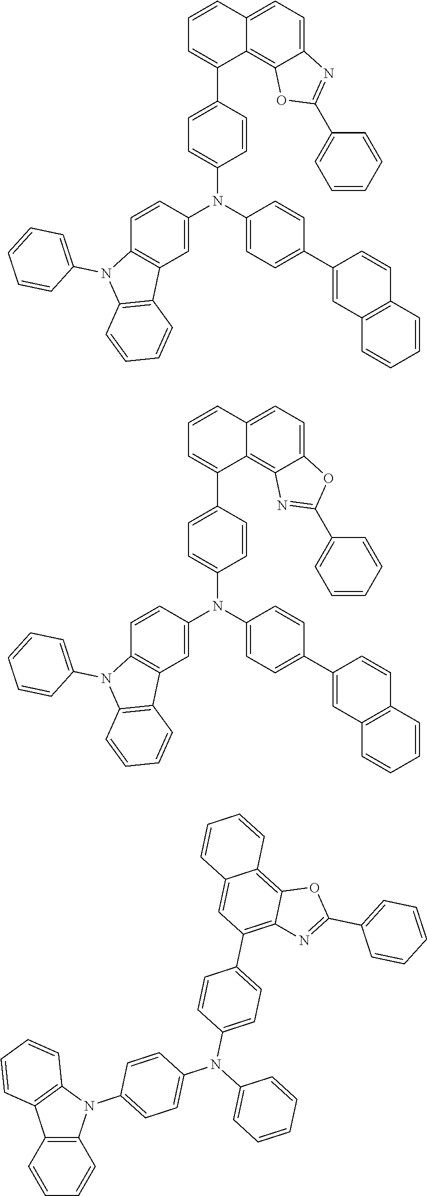 [SMILES: c1ccc(-c2nc3ccc4cccc(-c5ccc(N(c6ccc(-c7ccc8ccccc8c7)cc6)c6ccc7c(c6)c6ccccc6n7-c6ccccc6)cc5)c4c3o2)cc1 ; c1ccc(-c2nc3c(ccc4cccc(-c5ccc(N(c6ccc(-c7ccc8ccccc8c7)cc6)c6ccc7c(c6)c6ccccc6n7-c6ccccc6)cc5)c43)o2)cc1 ; c1ccc(-c2nc3c(-c4ccc(N(c5ccccc5)c5ccc(-n6c7ccccc7c7ccccc76)cc5)cc4)cc4ccccc4c3o2)cc1]

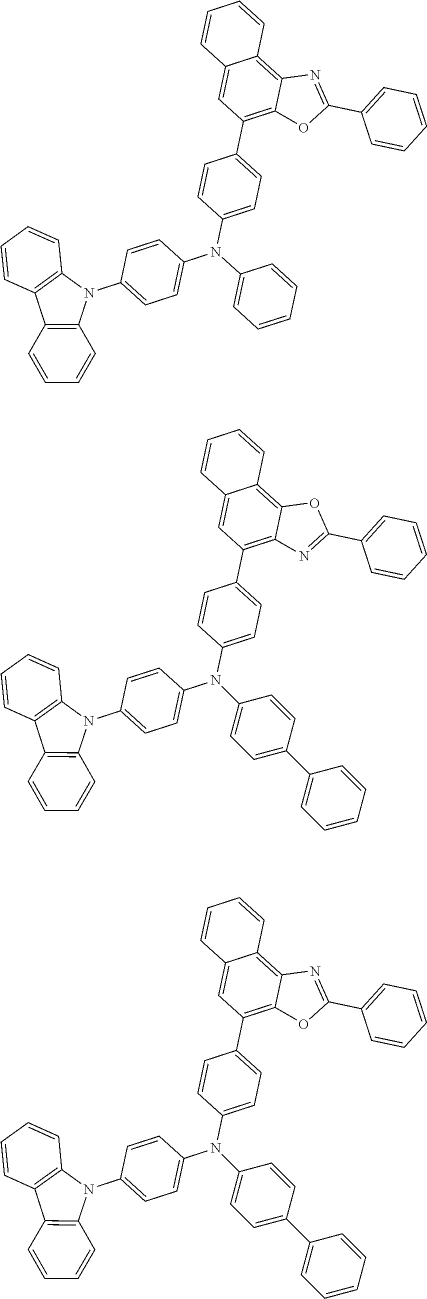 [SMILES: c1ccc(-c2nc3c(o2)c(-c2ccc(N(c4ccccc4)c4ccc(-n5c6ccccc6c6ccccc65)cc4)cc2)cc2ccccc23)cc1 ; c1ccc(-c2ccc(N(c3ccc(-c4cc5ccccc5c5oc(-c6ccccc6)nc45)cc3)c3ccc(-n4c5ccccc5c5ccccc54)cc3)cc2)cc1 ; c1ccc(-c2ccc(N(c3ccc(-c4cc5ccccc5c5nc(-c6ccccc6)oc45)cc3)c3ccc(-n4c5ccccc5c5ccccc54)cc3)cc2)cc1]

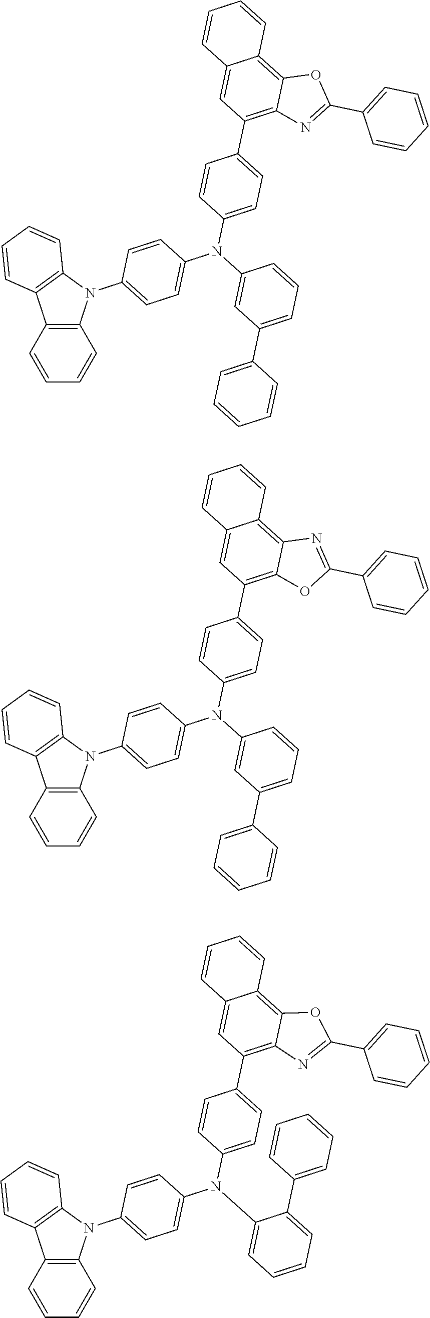

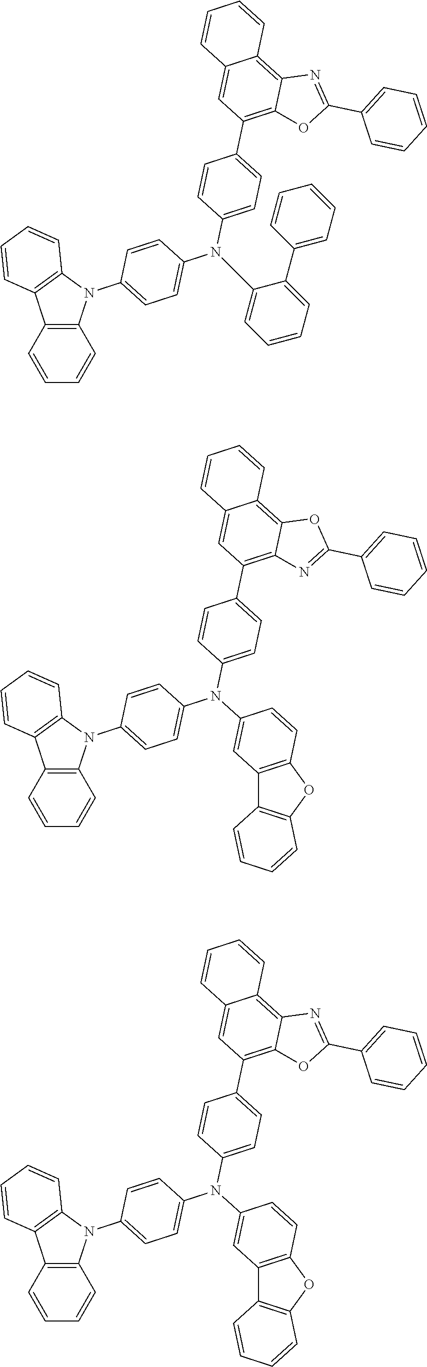 [SMILES: c1ccc(-c2nc3c(o2)c(-c2ccc(N(c4ccc(-n5c6ccccc6c6ccccc65)cc4)c4ccccc4-c4ccccc4)cc2)cc2ccccc23)cc1 ; c1ccc(-c2nc3c(-c4ccc(N(c5ccc(-n6c7ccccc7c7ccccc76)cc5)c5ccc6oc7ccccc7c6c5)cc4)cc4ccccc4c3o2)cc1 ; c1ccc(-c2nc3c(o2)c(-c2ccc(N(c4ccc(-n5c6ccccc6c6ccccc65)cc4)c4ccc5oc6ccccc6c5c4)cc2)cc2ccccc23)cc1]

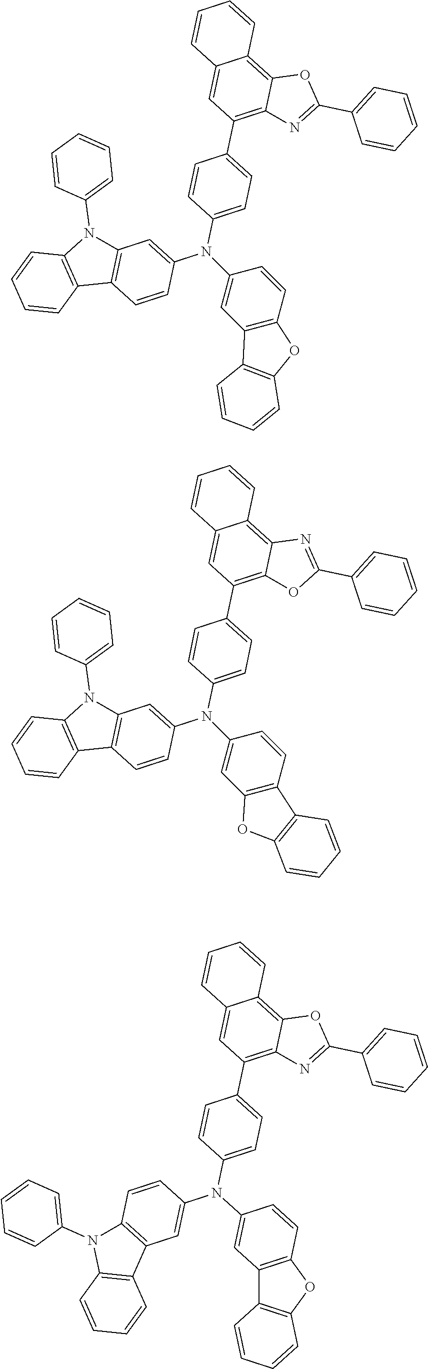

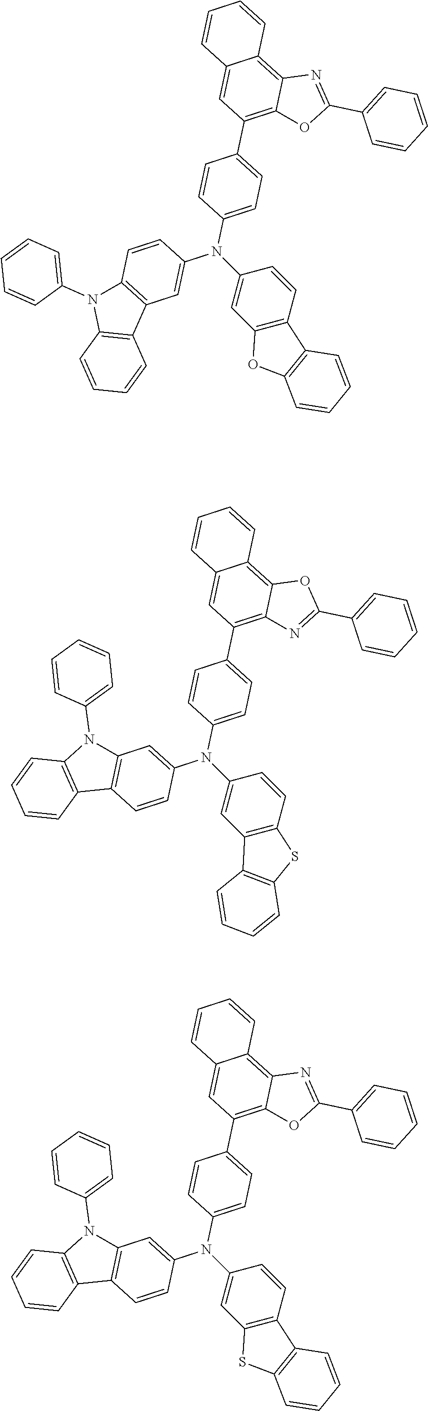 [SMILES: c1ccc(-c2nc3c(o2)c(-c2ccc(N(c4ccc5c(c4)oc4ccccc45)c4ccc5c(c4)c4ccccc4n5-c4ccccc4)cc2)cc2ccccc23)cc1 ; c1ccc(-c2nc3c(-c4ccc(N(c5ccc6sc7ccccc7c6c5)c5ccc6c7ccccc7n(-c7ccccc7)c6c5)cc4)cc4ccccc4c3o2)cc1 ; c1ccc(-c2nc3c(o2)c(-c2ccc(N(c4ccc5c(c4)sc4ccccc45)c4ccc5c6ccccc6n(-c6ccccc6)c5c4)cc2)cc2ccccc23)cc1]

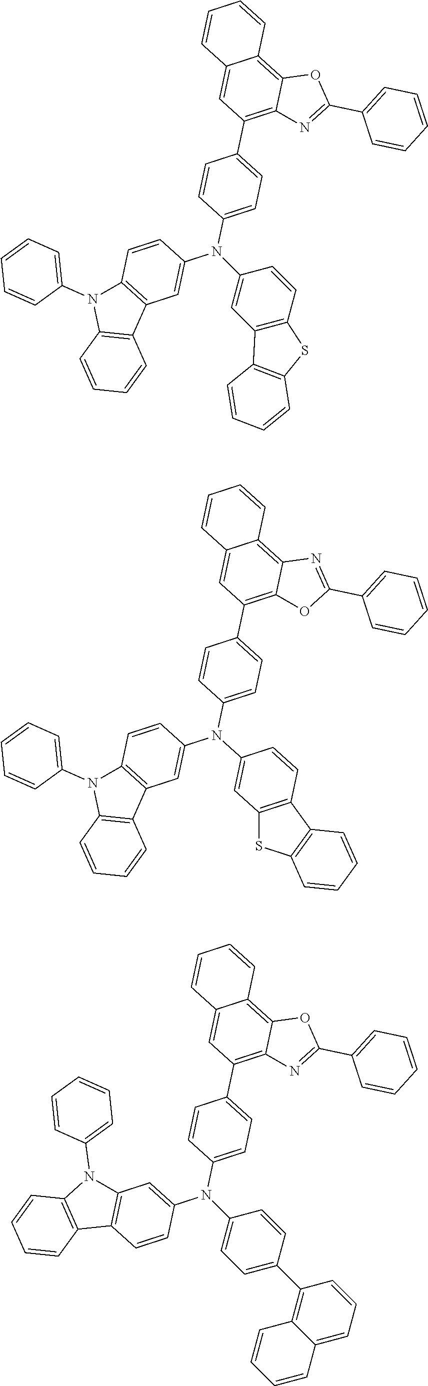 [SMILES: c1ccc(-c2nc3c(-c4ccc(N(c5ccc6sc7ccccc7c6c5)c5ccc6c(c5)c5ccccc5n6-c5ccccc5)cc4)cc4ccccc4c3o2)cc1 ; c1ccc(-c2nc3c(o2)c(-c2ccc(N(c4ccc5c(c4)sc4ccccc45)c4ccc5c(c4)c4ccccc4n5-c4ccccc4)cc2)cc2ccccc23)cc1 ; c1ccc(-c2nc3c(-c4ccc(N(c5ccc(-c6cccc7ccccc67)cc5)c5ccc6c7ccccc7n(-c7ccccc7)c6c5)cc4)cc4ccccc4c3o2)cc1]

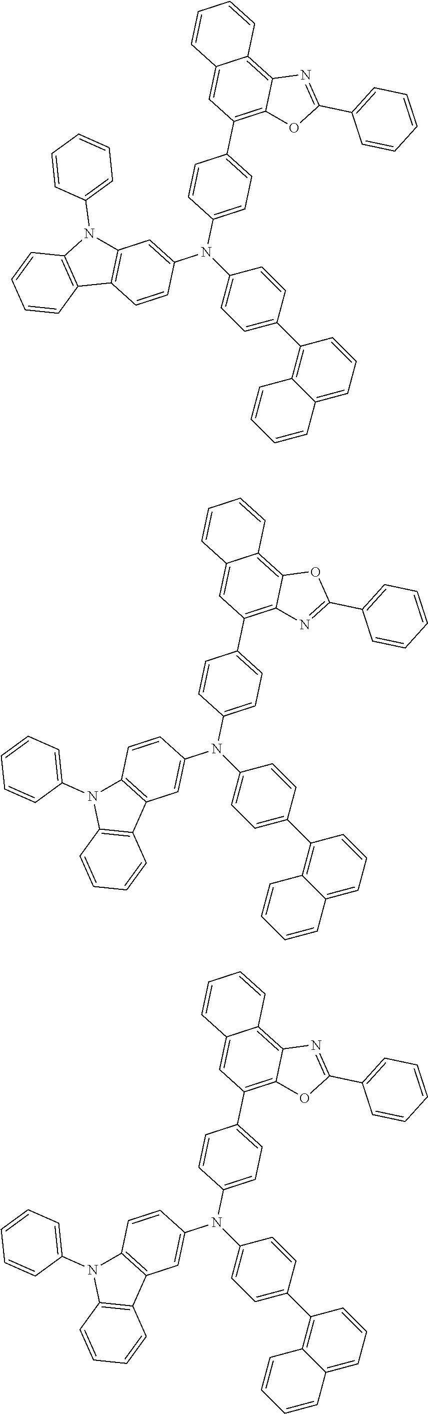 [SMILES: c1ccc(-c2nc3c(o2)c(-c2ccc(N(c4ccc(-c5cccc6ccccc56)cc4)c4ccc5c6ccccc6n(-c6ccccc6)c5c4)cc2)cc2ccccc23)cc1 ; c1ccc(-c2nc3c(-c4ccc(N(c5ccc(-c6cccc7ccccc67)cc5)c5ccc6c(c5)c5ccccc5n6-c5ccccc5)cc4)cc4ccccc4c3o2)cc1 ; c1ccc(-c2nc3c(o2)c(-c2ccc(N(c4ccc(-c5cccc6ccccc56)cc4)c4ccc5c(c4)c4ccccc4n5-c4ccccc4)cc2)cc2ccccc23)cc1]

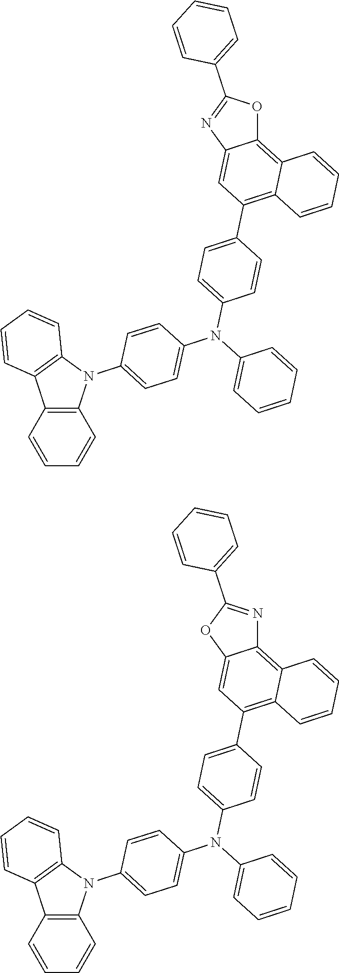 [SMILES: c1ccc(-c2nc3cc(-c4ccc(N(c5ccccc5)c5ccc(-n6c7ccccc7c7ccccc76)cc5)cc4)c4ccccc4c3o2)cc1 ; c1ccc(-c2nc3c(cc(-c4ccc(N(c5ccccc5)c5ccc(-n6c7ccccc7c7ccccc76)cc5)cc4)c4ccccc43)o2)cc1]

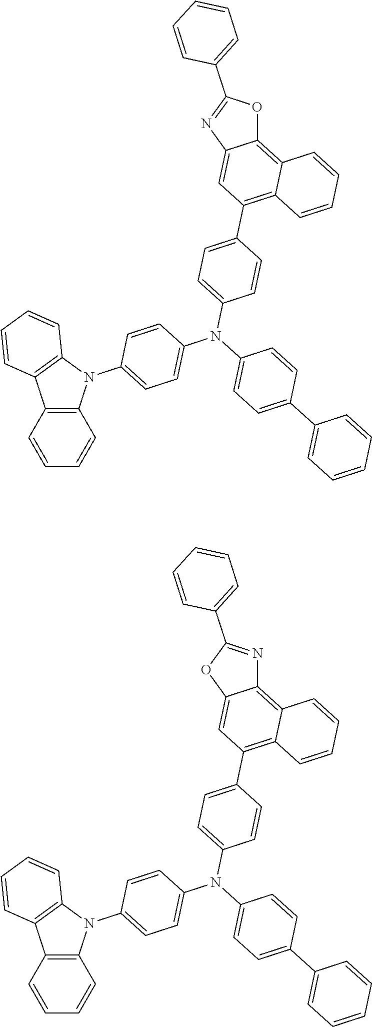 [SMILES: c1ccc(-c2ccc(N(c3ccc(-c4cc5nc(-c6ccccc6)oc5c5ccccc45)cc3)c3ccc(-n4c5ccccc5c5ccccc54)cc3)cc2)cc1 ; c1ccc(-c2ccc(N(c3ccc(-c4cc5oc(-c6ccccc6)nc5c5ccccc45)cc3)c3ccc(-n4c5ccccc5c5ccccc54)cc3)cc2)cc1]

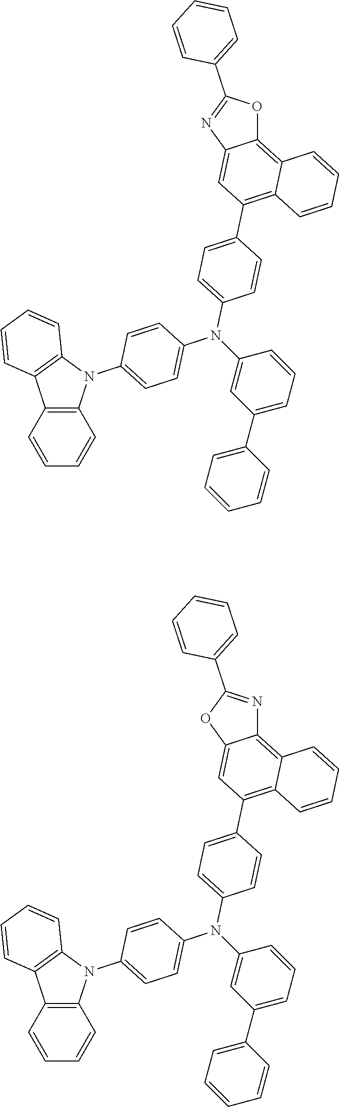 [SMILES: c1ccc(-c2cccc(N(c3ccc(-c4cc5nc(-c6ccccc6)oc5c5ccccc45)cc3)c3ccc(-n4c5ccccc5c5ccccc54)cc3)c2)cc1 ; c1ccc(-c2cccc(N(c3ccc(-c4cc5oc(-c6ccccc6)nc5c5ccccc45)cc3)c3ccc(-n4c5ccccc5c5ccccc54)cc3)c2)cc1]

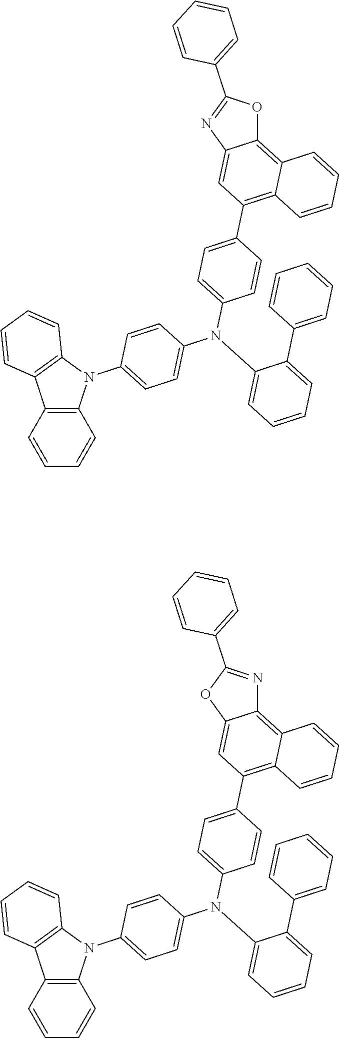 [SMILES: c1ccc(-c2nc3cc(-c4ccc(N(c5ccc(-n6c7ccccc7c7ccccc76)cc5)c5ccccc5-c5ccccc5)cc4)c4ccccc4c3o2)cc1 ; c1ccc(-c2nc3c(cc(-c4ccc(N(c5ccc(-n6c7ccccc7c7ccccc76)cc5)c5ccccc5-c5ccccc5)cc4)c4ccccc43)o2)cc1]

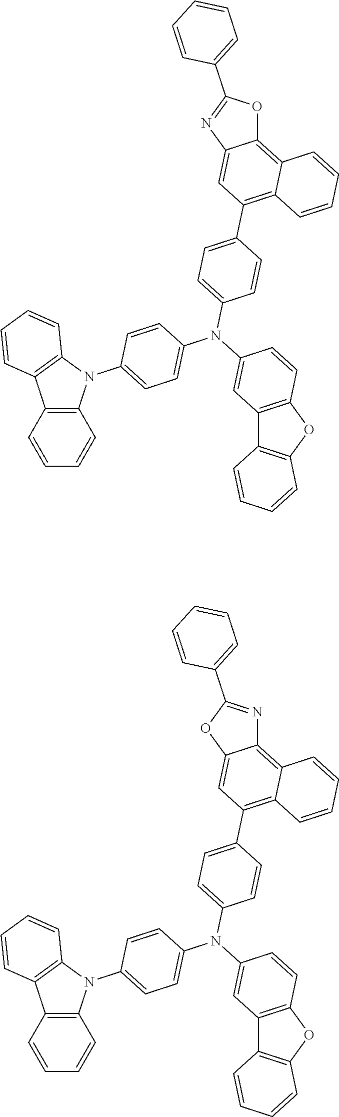 [SMILES: c1ccc(-c2nc3cc(-c4ccc(N(c5ccc(-n6c7ccccc7c7ccccc76)cc5)c5ccc6oc7ccccc7c6c5)cc4)c4ccccc4c3o2)cc1 ; c1ccc(-c2nc3c(cc(-c4ccc(N(c5ccc(-n6c7ccccc7c7ccccc76)cc5)c5ccc6oc7ccccc7c6c5)cc4)c4ccccc43)o2)cc1]

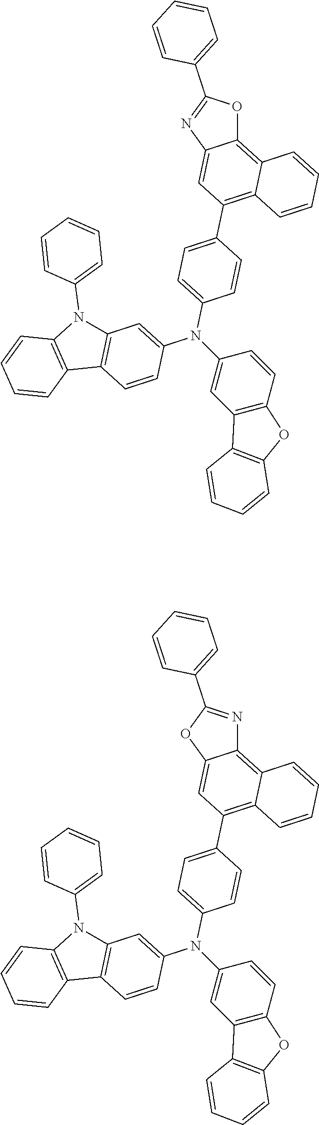 [SMILES: c1ccc(-c2nc3cc(-c4ccc(N(c5ccc6oc7ccccc7c6c5)c5ccc6c7ccccc7n(-c7ccccc7)c6c5)cc4)c4ccccc4c3o2)cc1 ; c1ccc(-c2nc3c(cc(-c4ccc(N(c5ccc6oc7ccccc7c6c5)c5ccc6c7ccccc7n(-c7ccccc7)c6c5)cc4)c4ccccc43)o2)cc1]

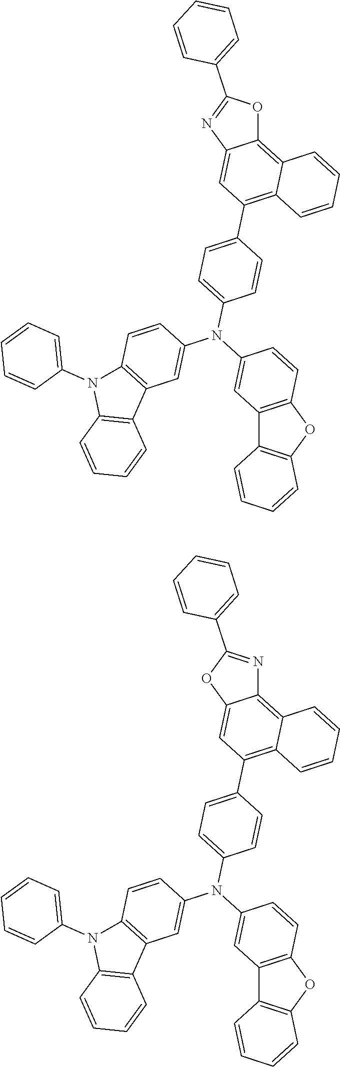 [SMILES: c1ccc(-c2nc3cc(-c4ccc(N(c5ccc6oc7ccccc7c6c5)c5ccc6c(c5)c5ccccc5n6-c5ccccc5)cc4)c4ccccc4c3o2)cc1 ; c1ccc(-c2nc3c(cc(-c4ccc(N(c5ccc6oc7ccccc7c6c5)c5ccc6c(c5)c5ccccc5n6-c5ccccc5)cc4)c4ccccc43)o2)cc1]

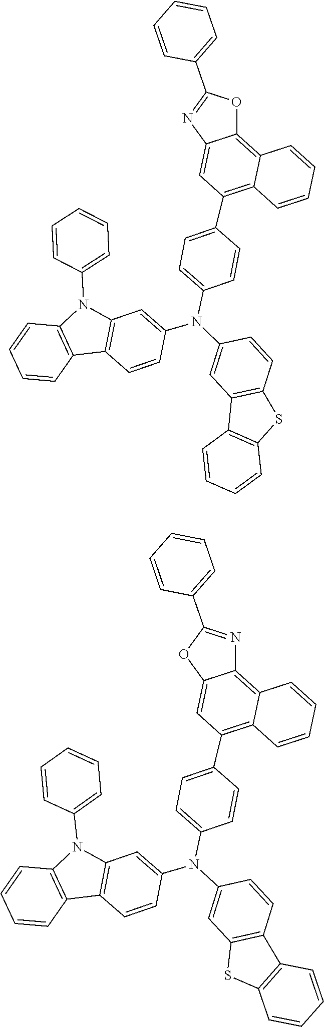 [SMILES: c1ccc(-c2nc3cc(-c4ccc(N(c5ccc6sc7ccccc7c6c5)c5ccc6c7ccccc7n(-c7ccccc7)c6c5)cc4)c4ccccc4c3o2)cc1 ; c1ccc(-c2nc3c(cc(-c4ccc(N(c5ccc6c(c5)sc5ccccc56)c5ccc6c7ccccc7n(-c7ccccc7)c6c5)cc4)c4ccccc43)o2)cc1]

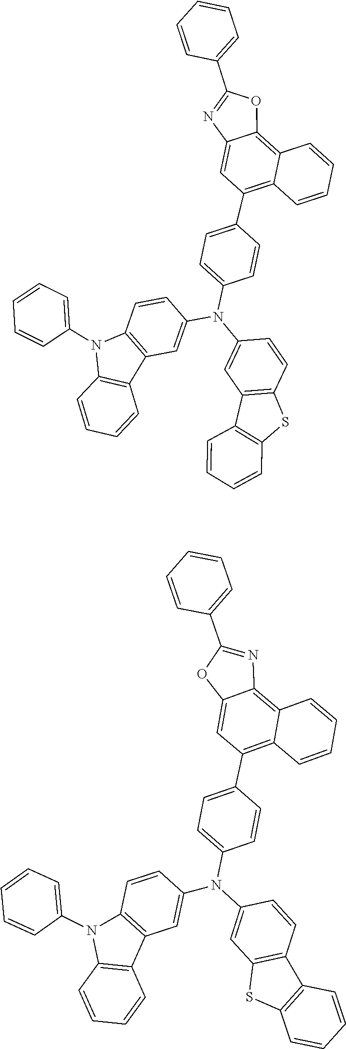 [SMILES: c1ccc(-c2nc3cc(-c4ccc(N(c5ccc6sc7ccccc7c6c5)c5ccc6c(c5)c5ccccc5n6-c5ccccc5)cc4)c4ccccc4c3o2)cc1 ; c1ccc(-c2nc3c(cc(-c4ccc(N(c5ccc6c(c5)sc5ccccc56)c5ccc6c(c5)c5ccccc5n6-c5ccccc5)cc4)c4ccccc43)o2)cc1]

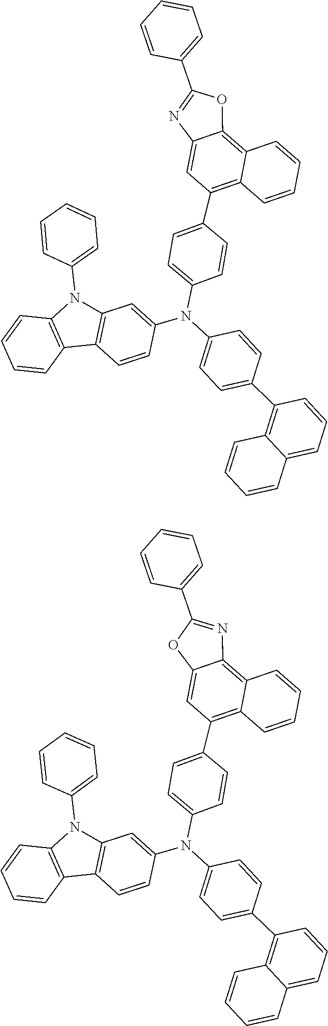 [SMILES: c1ccc(-c2nc3cc(-c4ccc(N(c5ccc(-c6cccc7ccccc67)cc5)c5ccc6c7ccccc7n(-c7ccccc7)c6c5)cc4)c4ccccc4c3o2)cc1 ; c1ccc(-c2nc3c(cc(-c4ccc(N(c5ccc(-c6cccc7ccccc67)cc5)c5ccc6c7ccccc7n(-c7ccccc7)c6c5)cc4)c4ccccc43)o2)cc1]

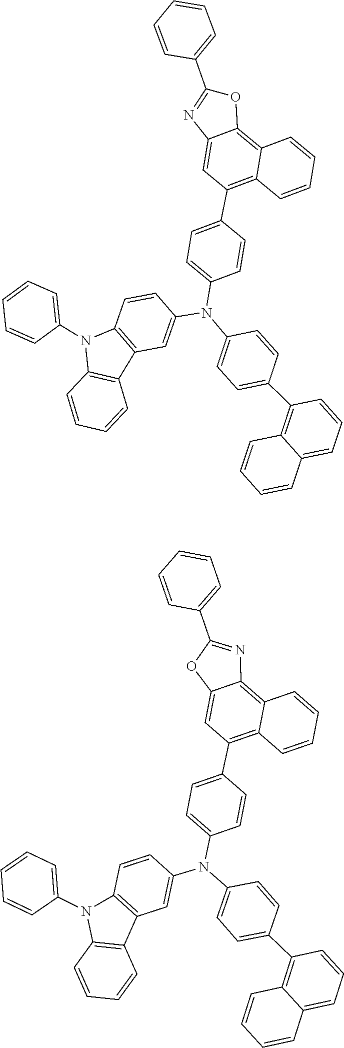 [SMILES: c1ccc(-c2nc3cc(-c4ccc(N(c5ccc(-c6cccc7ccccc67)cc5)c5ccc6c(c5)c5ccccc5n6-c5ccccc5)cc4)c4ccccc4c3o2)cc1 ; c1ccc(-c2nc3c(cc(-c4ccc(N(c5ccc(-c6cccc7ccccc67)cc5)c5ccc6c(c5)c5ccccc5n6-c5ccccc5)cc4)c4ccccc43)o2)cc1]

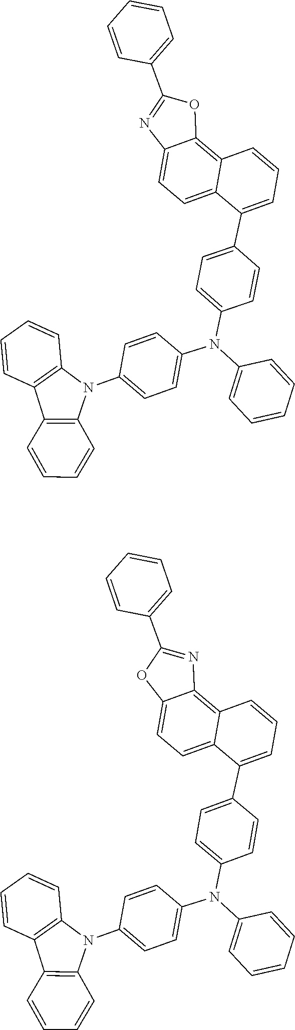 [SMILES: c1ccc(-c2nc3ccc4c(-c5ccc(N(c6ccccc6)c6ccc(-n7c8ccccc8c8ccccc87)cc6)cc5)cccc4c3o2)cc1 ; c1ccc(-c2nc3c(ccc4c(-c5ccc(N(c6ccccc6)c6ccc(-n7c8ccccc8c8ccccc87)cc6)cc5)cccc43)o2)cc1]

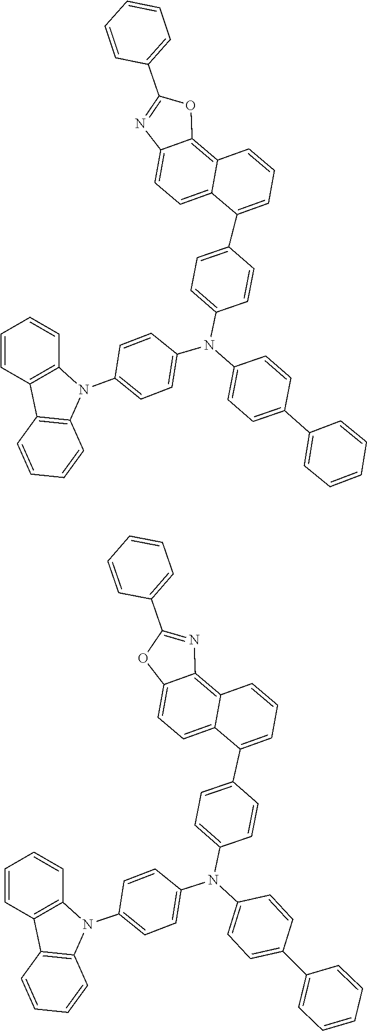 [SMILES: c1ccc(-c2ccc(N(c3ccc(-c4cccc5c4ccc4nc(-c6ccccc6)oc45)cc3)c3ccc(-n4c5ccccc5c5ccccc54)cc3)cc2)cc1 ; c1ccc(-c2ccc(N(c3ccc(-c4cccc5c4ccc4oc(-c6ccccc6)nc45)cc3)c3ccc(-n4c5ccccc5c5ccccc54)cc3)cc2)cc1]

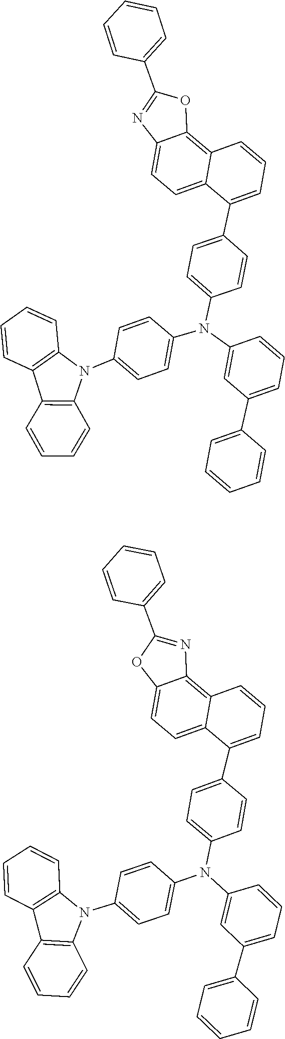 [SMILES: c1ccc(-c2cccc(N(c3ccc(-c4cccc5c4ccc4nc(-c6ccccc6)oc45)cc3)c3ccc(-n4c5ccccc5c5ccccc54)cc3)c2)cc1 ; c1ccc(-c2cccc(N(c3ccc(-c4cccc5c4ccc4oc(-c6ccccc6)nc45)cc3)c3ccc(-n4c5ccccc5c5ccccc54)cc3)c2)cc1]

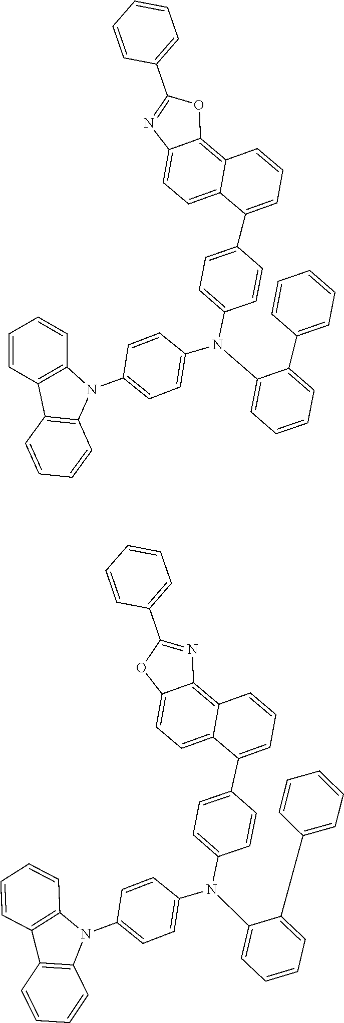 [SMILES: c1ccc(-c2nc3ccc4c(-c5ccc(N(c6ccc(-n7c8ccccc8c8ccccc87)cc6)c6ccccc6-c6ccccc6)cc5)cccc4c3o2)cc1 ; c1ccc(-c2nc3c(ccc4c(-c5ccc(N(c6ccc(-n7c8ccccc8c8ccccc87)cc6)c6ccccc6-c6ccccc6)cc5)cccc43)o2)cc1]

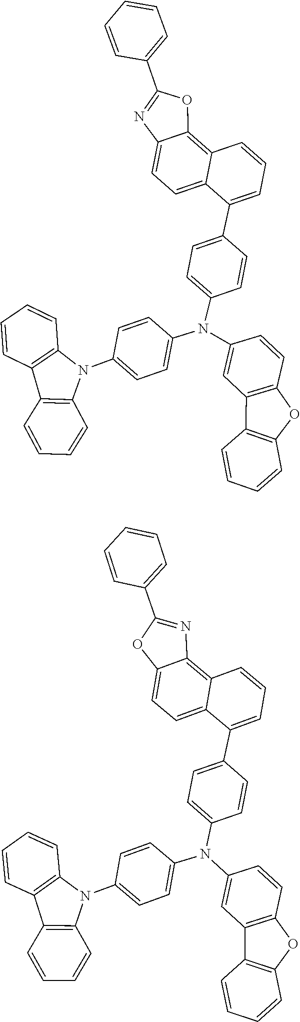 [SMILES: c1ccc(-c2nc3ccc4c(-c5ccc(N(c6ccc(-n7c8ccccc8c8ccccc87)cc6)c6ccc7oc8ccccc8c7c6)cc5)cccc4c3o2)cc1 ; c1ccc(-c2nc3c(ccc4c(-c5ccc(N(c6ccc(-n7c8ccccc8c8ccccc87)cc6)c6ccc7oc8ccccc8c7c6)cc5)cccc43)o2)cc1]

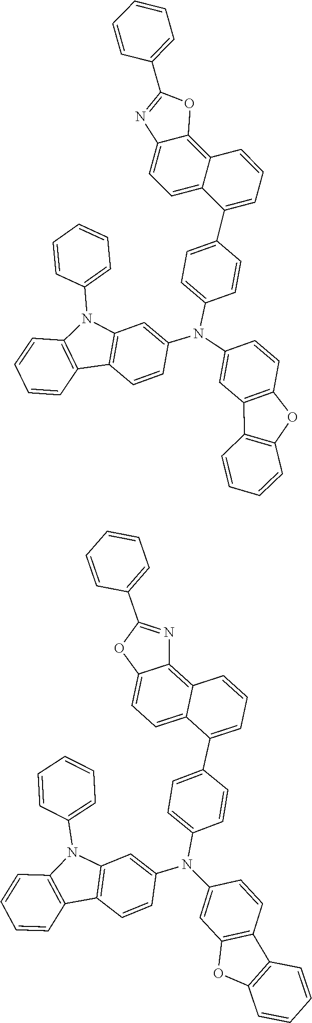 [SMILES: c1ccc(-c2nc3ccc4c(-c5ccc(N(c6ccc7oc8ccccc8c7c6)c6ccc7c8ccccc8n(-c8ccccc8)c7c6)cc5)cccc4c3o2)cc1 ; c1ccc(-c2nc3c(ccc4c(-c5ccc(N(c6ccc7c(c6)oc6ccccc67)c6ccc7c8ccccc8n(-c8ccccc8)c7c6)cc5)cccc43)o2)cc1]

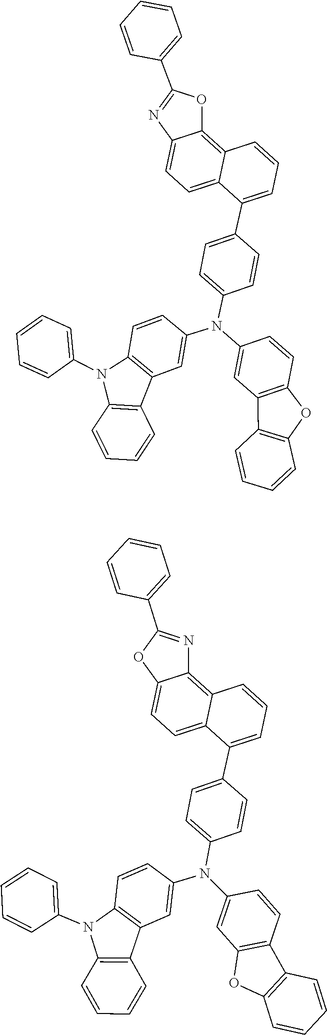 [SMILES: c1ccc(-c2nc3ccc4c(-c5ccc(N(c6ccc7oc8ccccc8c7c6)c6ccc7c(c6)c6ccccc6n7-c6ccccc6)cc5)cccc4c3o2)cc1 ; c1ccc(-c2nc3c(ccc4c(-c5ccc(N(c6ccc7c(c6)oc6ccccc67)c6ccc7c(c6)c6ccccc6n7-c6ccccc6)cc5)cccc43)o2)cc1]

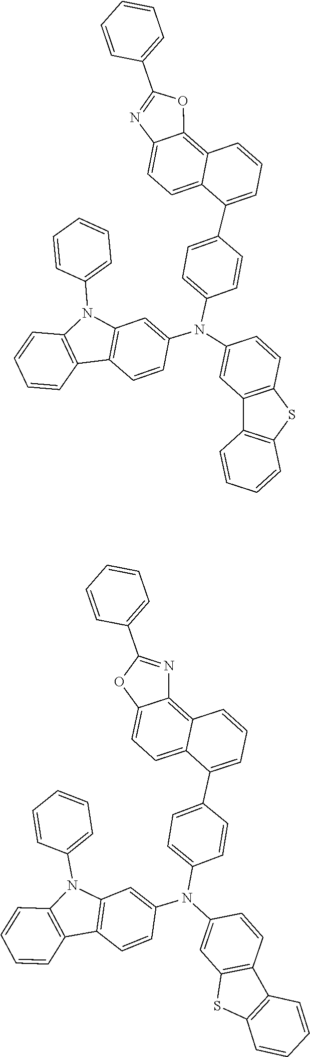 [SMILES: c1ccc(-c2nc3ccc4c(-c5ccc(N(c6ccc7sc8ccccc8c7c6)c6ccc7c8ccccc8n(-c8ccccc8)c7c6)cc5)cccc4c3o2)cc1 ; c1ccc(-c2nc3c(ccc4c(-c5ccc(N(c6ccc7c(c6)sc6ccccc67)c6ccc7c8ccccc8n(-c8ccccc8)c7c6)cc5)cccc43)o2)cc1]

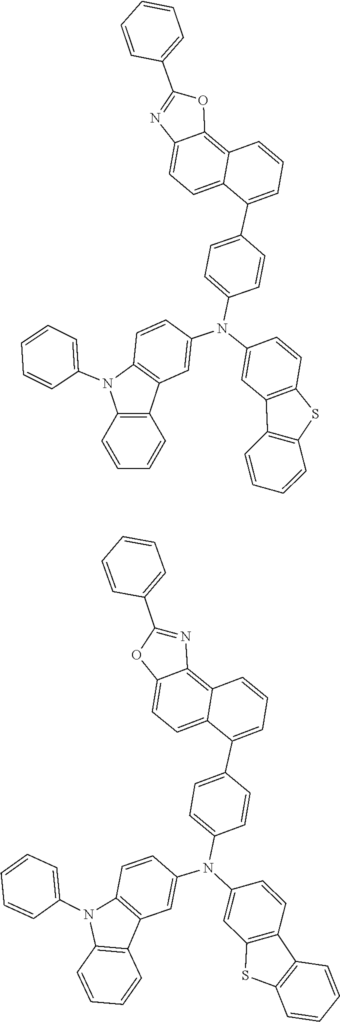 [SMILES: c1ccc(-c2nc3ccc4c(-c5ccc(N(c6ccc7sc8ccccc8c7c6)c6ccc7c(c6)c6ccccc6n7-c6ccccc6)cc5)cccc4c3o2)cc1 ; c1ccc(-c2nc3c(ccc4c(-c5ccc(N(c6ccc7c(c6)sc6ccccc67)c6ccc7c(c6)c6ccccc6n7-c6ccccc6)cc5)cccc43)o2)cc1]

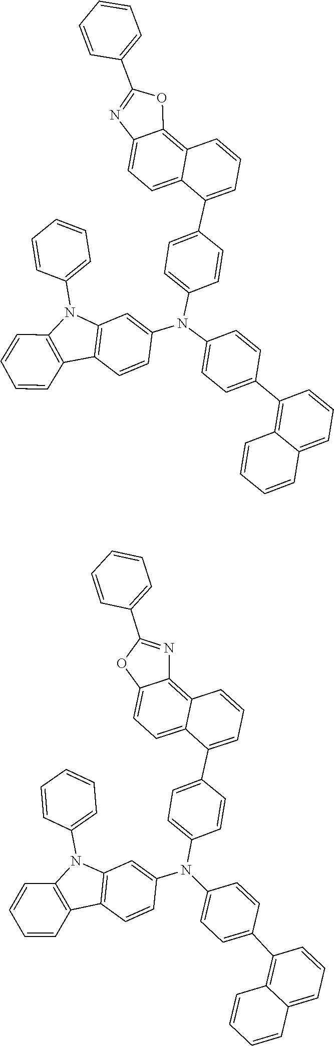 [SMILES: c1ccc(-c2nc3ccc4c(-c5ccc(N(c6ccc(-c7cccc8ccccc78)cc6)c6ccc7c8ccccc8n(-c8ccccc8)c7c6)cc5)cccc4c3o2)cc1 ; c1ccc(-c2nc3c(ccc4c(-c5ccc(N(c6ccc(-c7cccc8ccccc78)cc6)c6ccc7c8ccccc8n(-c8ccccc8)c7c6)cc5)cccc43)o2)cc1]

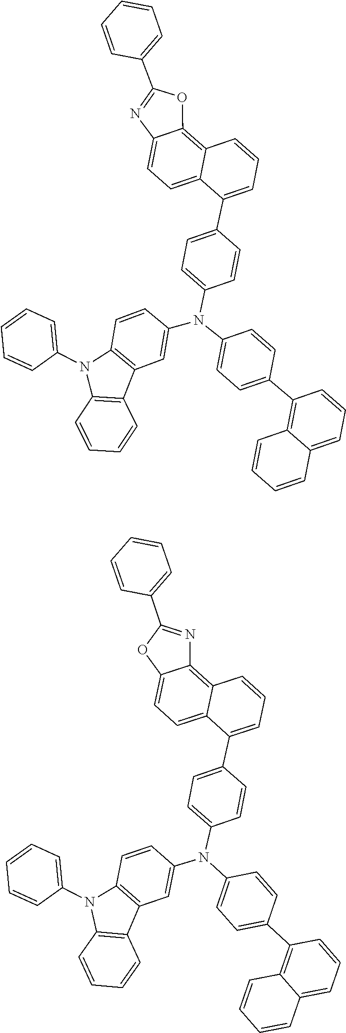 [SMILES: c1ccc(-c2nc3ccc4c(-c5ccc(N(c6ccc(-c7cccc8ccccc78)cc6)c6ccc7c(c6)c6ccccc6n7-c6ccccc6)cc5)cccc4c3o2)cc1 ; c1ccc(-c2nc3c(ccc4c(-c5ccc(N(c6ccc(-c7cccc8ccccc78)cc6)c6ccc7c(c6)c6ccccc6n7-c6ccccc6)cc5)cccc43)o2)cc1]

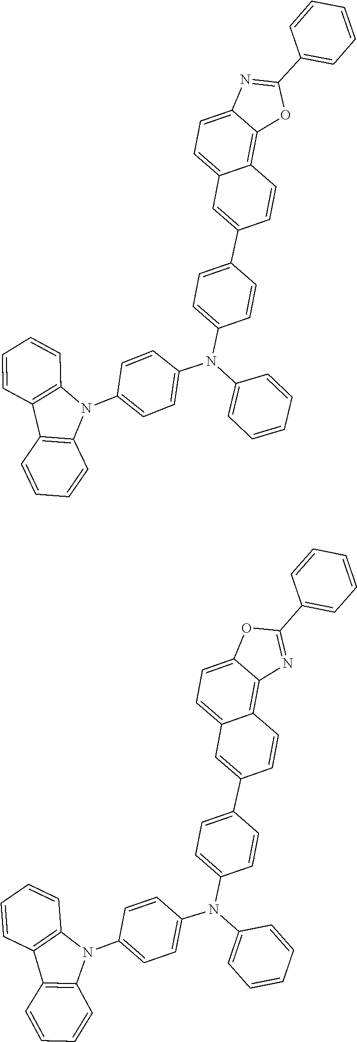 [SMILES: c1ccc(-c2nc3ccc4cc(-c5ccc(N(c6ccccc6)c6ccc(-n7c8ccccc8c8ccccc87)cc6)cc5)ccc4c3o2)cc1 ; c1ccc(-c2nc3c(ccc4cc(-c5ccc(N(c6ccccc6)c6ccc(-n7c8ccccc8c8ccccc87)cc6)cc5)ccc43)o2)cc1]

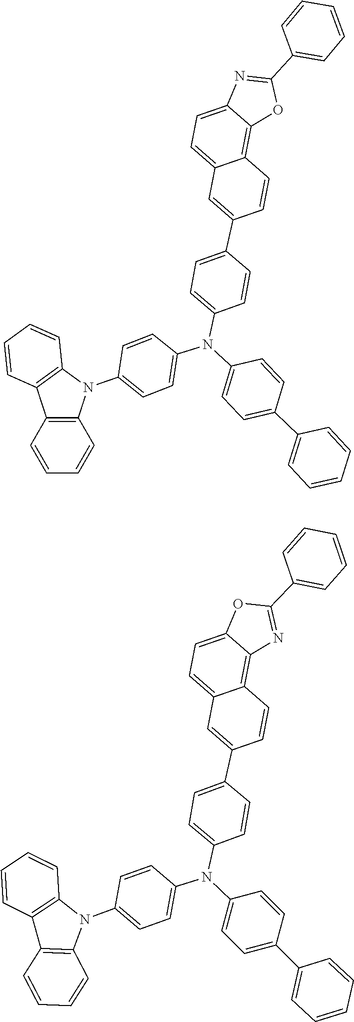

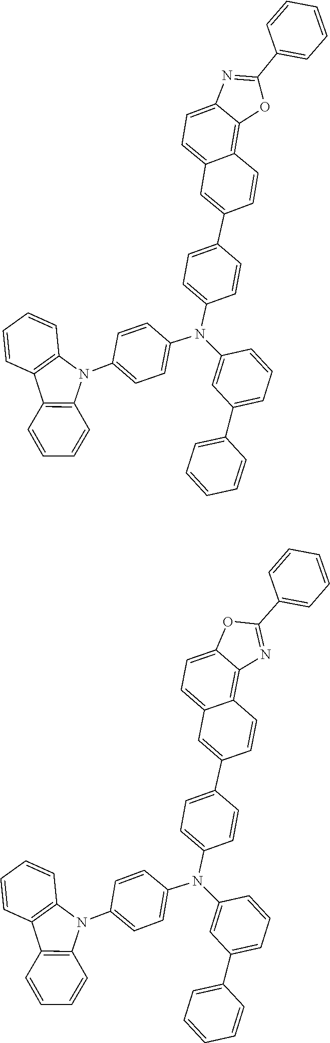 [SMILES: c1ccc(-c2cccc(N(c3ccc(-c4ccc5c(ccc6nc(-c7ccccc7)oc65)c4)cc3)c3ccc(-n4c5ccccc5c5ccccc54)cc3)c2)cc1 ; c1ccc(-c2cccc(N(c3ccc(-c4ccc5c(ccc6oc(-c7ccccc7)nc65)c4)cc3)c3ccc(-n4c5ccccc5c5ccccc54)cc3)c2)cc1]

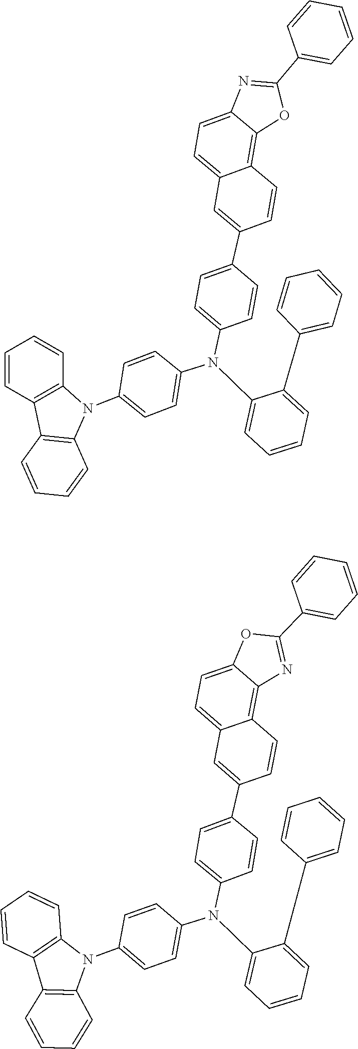 [SMILES: c1ccc(-c2nc3ccc4cc(-c5ccc(N(c6ccc(-n7c8ccccc8c8ccccc87)cc6)c6ccccc6-c6ccccc6)cc5)ccc4c3o2)cc1 ; c1ccc(-c2nc3c(ccc4cc(-c5ccc(N(c6ccc(-n7c8ccccc8c8ccccc87)cc6)c6ccccc6-c6ccccc6)cc5)ccc43)o2)cc1]

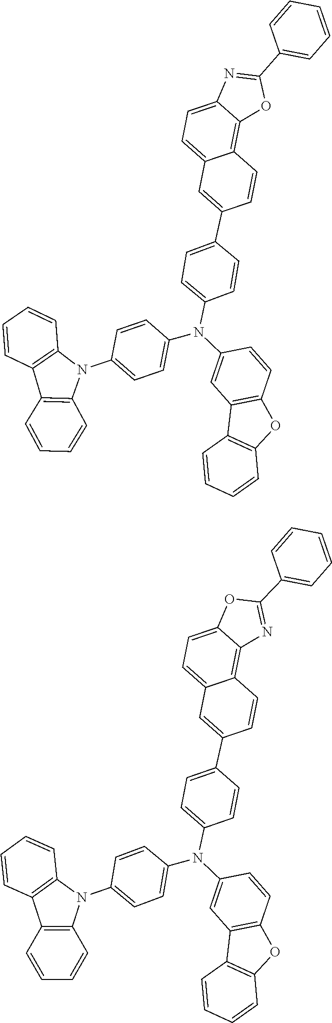 [SMILES: c1ccc(-c2nc3ccc4cc(-c5ccc(N(c6ccc(-n7c8ccccc8c8ccccc87)cc6)c6ccc7oc8ccccc8c7c6)cc5)ccc4c3o2)cc1 ; c1ccc(-c2nc3c(ccc4cc(-c5ccc(N(c6ccc(-n7c8ccccc8c8ccccc87)cc6)c6ccc7oc8ccccc8c7c6)cc5)ccc43)o2)cc1]

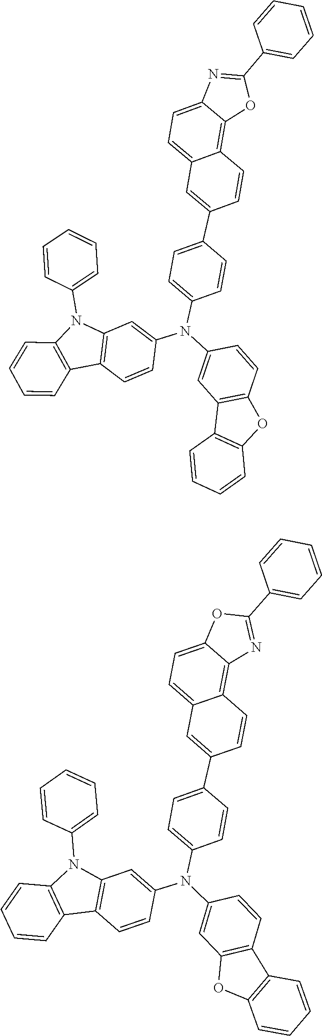 [SMILES: c1ccc(-c2nc3ccc4cc(-c5ccc(N(c6ccc7oc8ccccc8c7c6)c6ccc7c8ccccc8n(-c8ccccc8)c7c6)cc5)ccc4c3o2)cc1 ; c1ccc(-c2nc3c(ccc4cc(-c5ccc(N(c6ccc7c(c6)oc6ccccc67)c6ccc7c8ccccc8n(-c8ccccc8)c7c6)cc5)ccc43)o2)cc1]

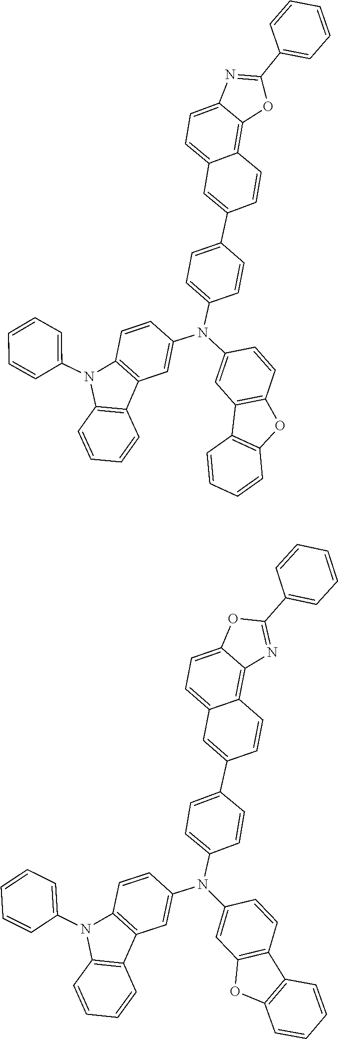 [SMILES: c1ccc(-c2nc3ccc4cc(-c5ccc(N(c6ccc7oc8ccccc8c7c6)c6ccc7c(c6)c6ccccc6n7-c6ccccc6)cc5)ccc4c3o2)cc1 ; c1ccc(-c2nc3c(ccc4cc(-c5ccc(N(c6ccc7c(c6)oc6ccccc67)c6ccc7c(c6)c6ccccc6n7-c6ccccc6)cc5)ccc43)o2)cc1]

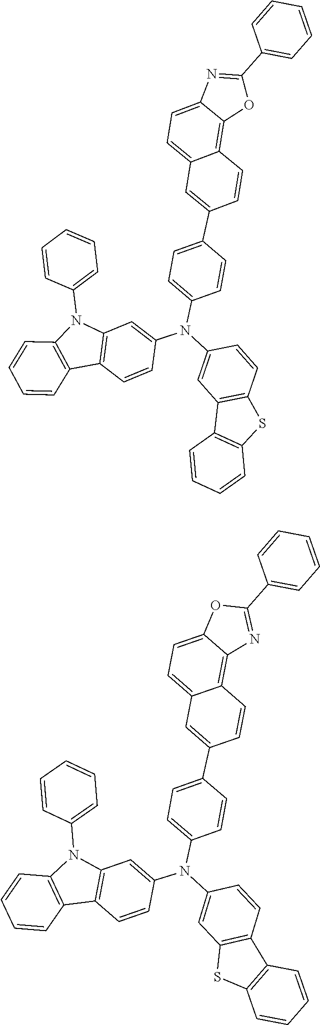 [SMILES: c1ccc(-c2nc3ccc4cc(-c5ccc(N(c6ccc7sc8ccccc8c7c6)c6ccc7c8ccccc8n(-c8ccccc8)c7c6)cc5)ccc4c3o2)cc1 ; c1ccc(-c2nc3c(ccc4cc(-c5ccc(N(c6ccc7c(c6)sc6ccccc67)c6ccc7c8ccccc8n(-c8ccccc8)c7c6)cc5)ccc43)o2)cc1]

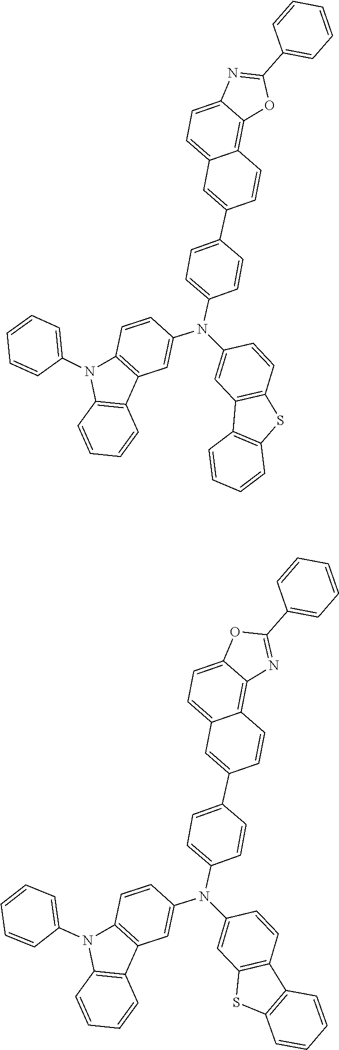 [SMILES: c1ccc(-c2nc3ccc4cc(-c5ccc(N(c6ccc7sc8ccccc8c7c6)c6ccc7c(c6)c6ccccc6n7-c6ccccc6)cc5)ccc4c3o2)cc1 ; c1ccc(-c2nc3c(ccc4cc(-c5ccc(N(c6ccc7c(c6)sc6ccccc67)c6ccc7c(c6)c6ccccc6n7-c6ccccc6)cc5)ccc43)o2)cc1]

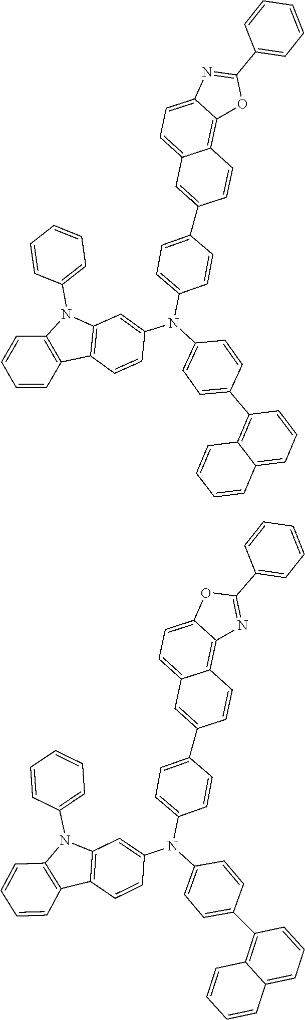

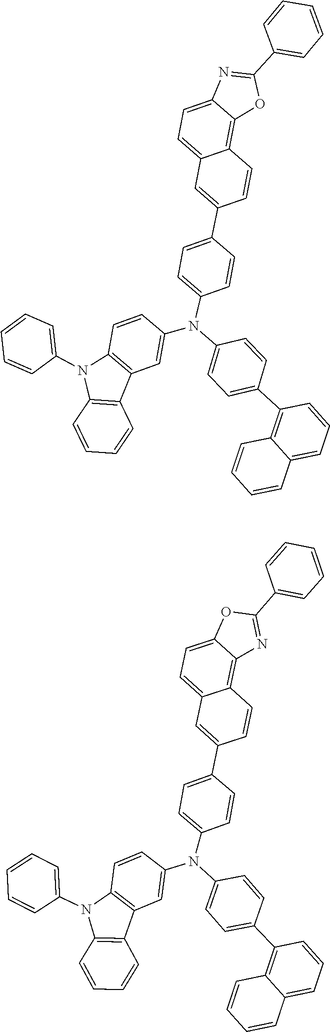 [SMILES: c1ccc(-c2nc3ccc4cc(-c5ccc(N(c6ccc(-c7cccc8ccccc78)cc6)c6ccc7c(c6)c6ccccc6n7-c6ccccc6)cc5)ccc4c3o2)cc1 ; c1ccc(-c2nc3c(ccc4cc(-c5ccc(N(c6ccc(-c7cccc8ccccc78)cc6)c6ccc7c(c6)c6ccccc6n7-c6ccccc6)cc5)ccc43)o2)cc1]

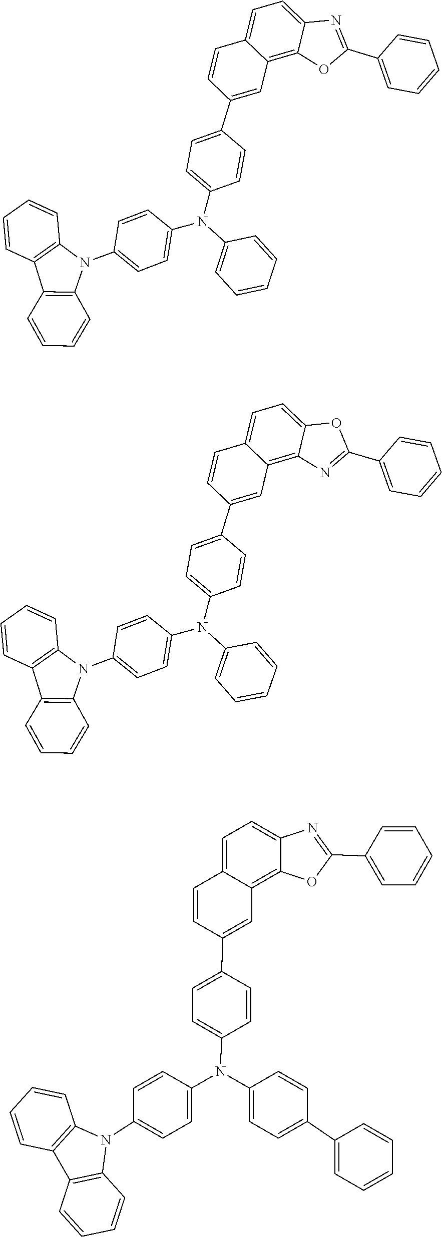 [SMILES: c1ccc(-c2nc3ccc4ccc(-c5ccc(N(c6ccccc6)c6ccc(-n7c8ccccc8c8ccccc87)cc6)cc5)cc4c3o2)cc1 ; c1ccc(-c2nc3c(ccc4ccc(-c5ccc(N(c6ccccc6)c6ccc(-n7c8ccccc8c8ccccc87)cc6)cc5)cc43)o2)cc1 ; c1ccc(-c2ccc(N(c3ccc(-c4ccc5ccc6nc(-c7ccccc7)oc6c5c4)cc3)c3ccc(-n4c5ccccc5c5ccccc54)cc3)cc2)cc1]

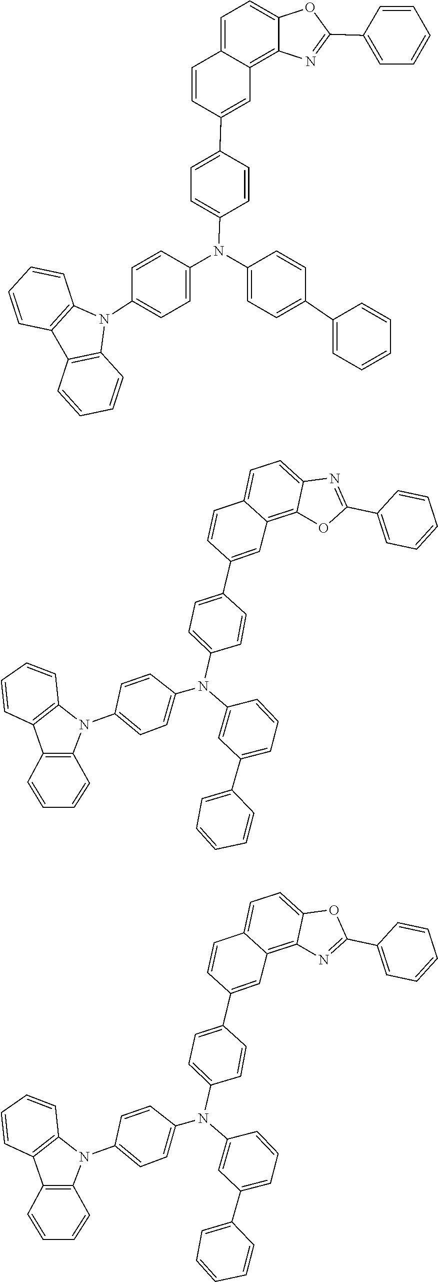 [SMILES: c1ccc(-c2ccc(N(c3ccc(-c4ccc5ccc6oc(-c7ccccc7)nc6c5c4)cc3)c3ccc(-n4c5ccccc5c5ccccc54)cc3)cc2)cc1 ; c1ccc(-c2cccc(N(c3ccc(-c4ccc5ccc6nc(-c7ccccc7)oc6c5c4)cc3)c3ccc(-n4c5ccccc5c5ccccc54)cc3)c2)cc1 ; c1ccc(-c2cccc(N(c3ccc(-c4ccc5ccc6oc(-c7ccccc7)nc6c5c4)cc3)c3ccc(-n4c5ccccc5c5ccccc54)cc3)c2)cc1]

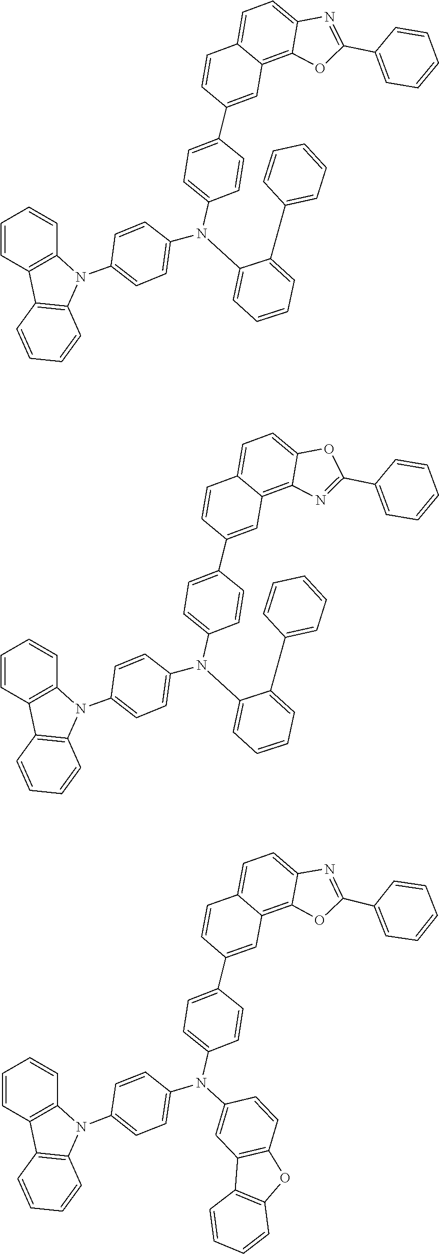 [SMILES: c1ccc(-c2nc3ccc4ccc(-c5ccc(N(c6ccc(-n7c8ccccc8c8ccccc87)cc6)c6ccccc6-c6ccccc6)cc5)cc4c3o2)cc1 ; c1ccc(-c2nc3c(ccc4ccc(-c5ccc(N(c6ccc(-n7c8ccccc8c8ccccc87)cc6)c6ccccc6-c6ccccc6)cc5)cc43)o2)cc1 ; c1ccc(-c2nc3ccc4ccc(-c5ccc(N(c6ccc(-n7c8ccccc8c8ccccc87)cc6)c6ccc7oc8ccccc8c7c6)cc5)cc4c3o2)cc1]

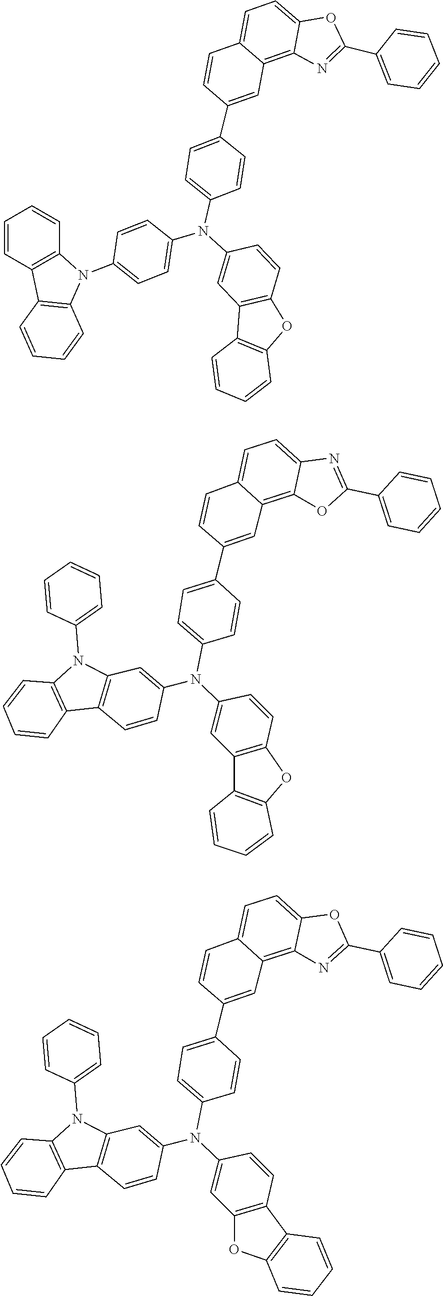 [SMILES: c1ccc(-c2nc3c(ccc4ccc(-c5ccc(N(c6ccc(-n7c8ccccc8c8ccccc87)cc6)c6ccc7oc8ccccc8c7c6)cc5)cc43)o2)cc1 ; c1ccc(-c2nc3ccc4ccc(-c5ccc(N(c6ccc7oc8ccccc8c7c6)c6ccc7c8ccccc8n(-c8ccccc8)c7c6)cc5)cc4c3o2)cc1 ; c1ccc(-c2nc3c(ccc4ccc(-c5ccc(N(c6ccc7c(c6)oc6ccccc67)c6ccc7c8ccccc8n(-c8ccccc8)c7c6)cc5)cc43)o2)cc1]

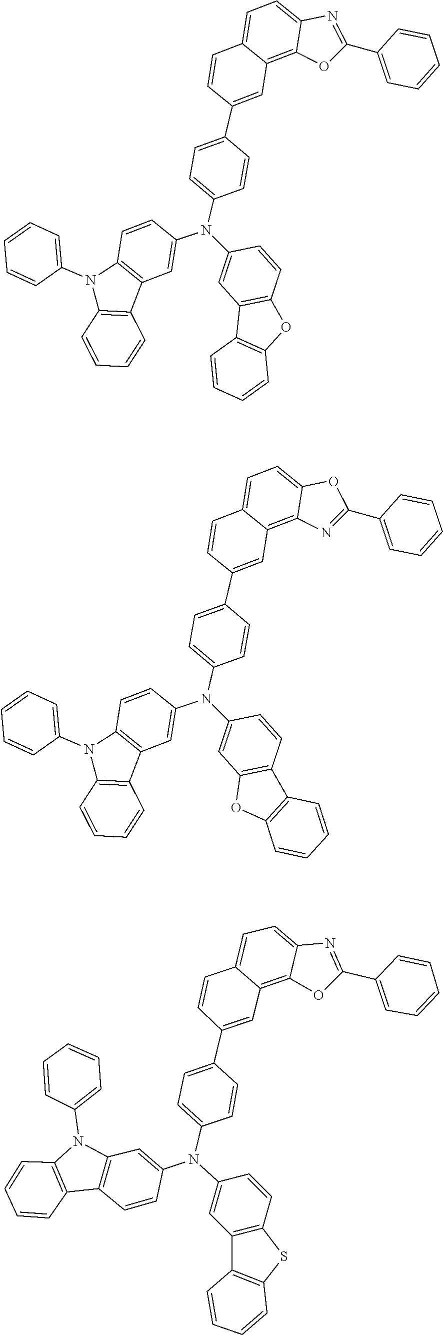 [SMILES: c1ccc(-c2nc3ccc4ccc(-c5ccc(N(c6ccc7oc8ccccc8c7c6)c6ccc7c(c6)c6ccccc6n7-c6ccccc6)cc5)cc4c3o2)cc1 ; c1ccc(-c2nc3c(ccc4ccc(-c5ccc(N(c6ccc7c(c6)oc6ccccc67)c6ccc7c(c6)c6ccccc6n7-c6ccccc6)cc5)cc43)o2)cc1 ; c1ccc(-c2nc3ccc4ccc(-c5ccc(N(c6ccc7sc8ccccc8c7c6)c6ccc7c8ccccc8n(-c8ccccc8)c7c6)cc5)cc4c3o2)cc1]

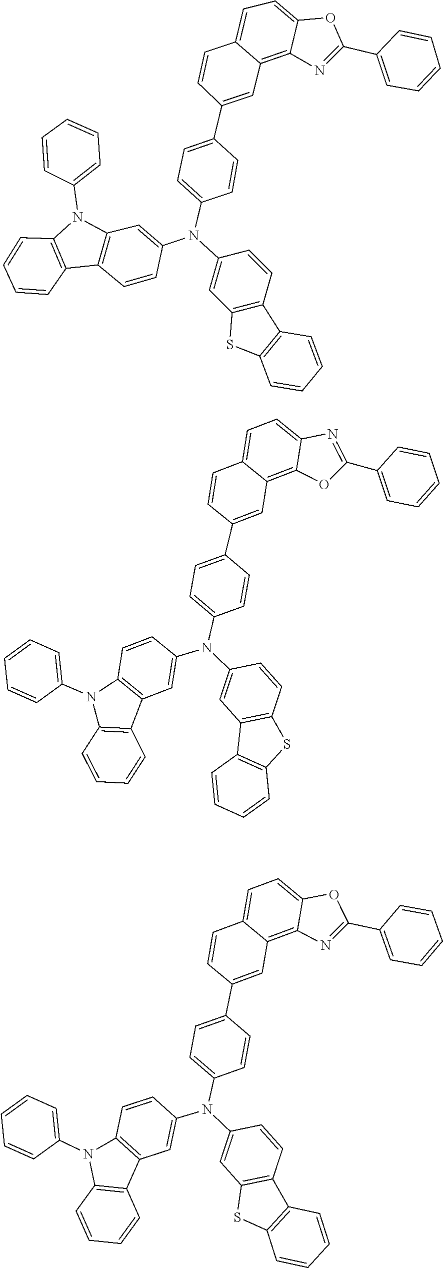

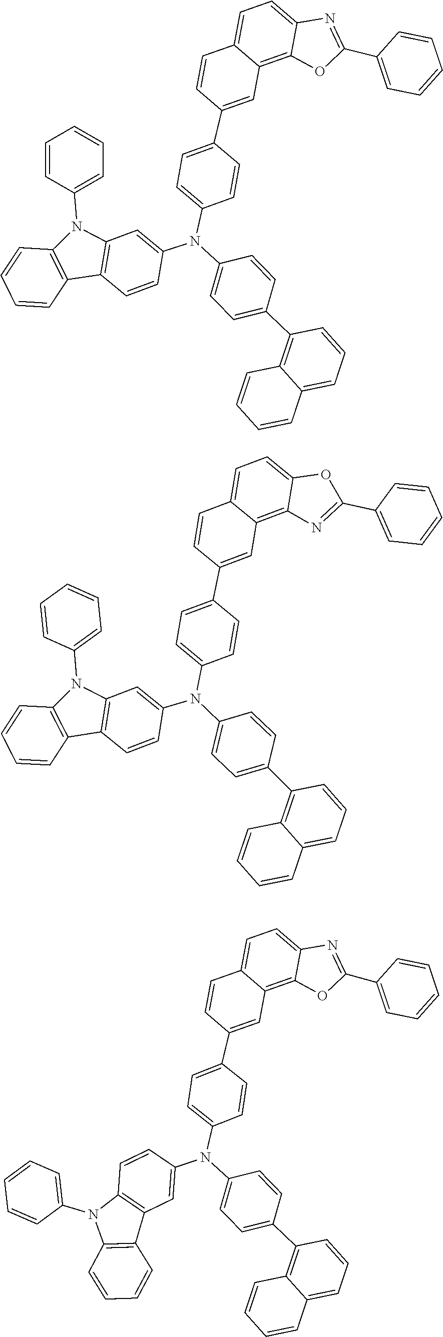

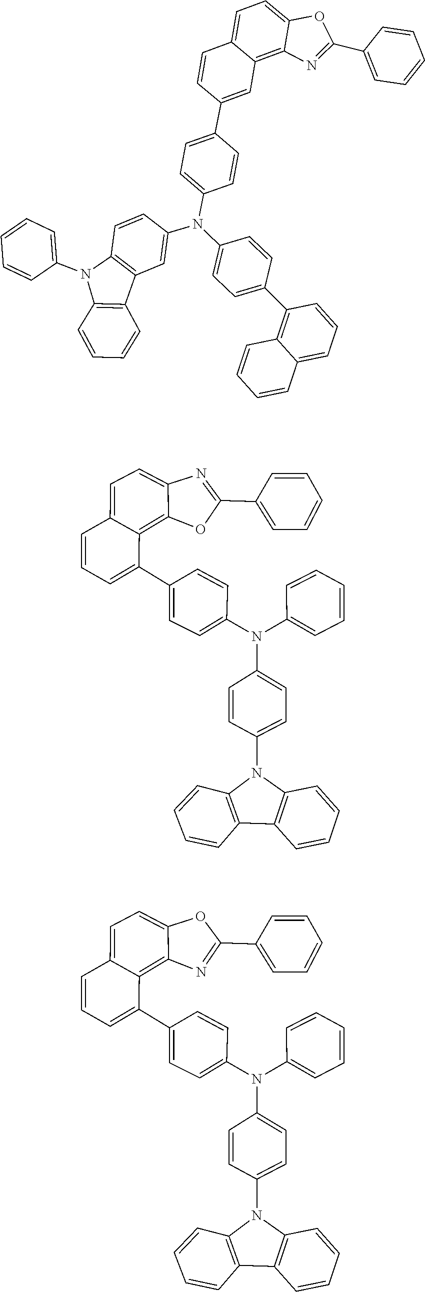 [SMILES: c1ccc(-c2nc3c(ccc4ccc(-c5ccc(N(c6ccc(-c7cccc8ccccc78)cc6)c6ccc7c(c6)c6ccccc6n7-c6ccccc6)cc5)cc43)o2)cc1 ; c1ccc(-c2nc3ccc4cccc(-c5ccc(N(c6ccccc6)c6ccc(-n7c8ccccc8c8ccccc87)cc6)cc5)c4c3o2)cc1 ; c1ccc(-c2nc3c(ccc4cccc(-c5ccc(N(c6ccccc6)c6ccc(-n7c8ccccc8c8ccccc87)cc6)cc5)c43)o2)cc1]

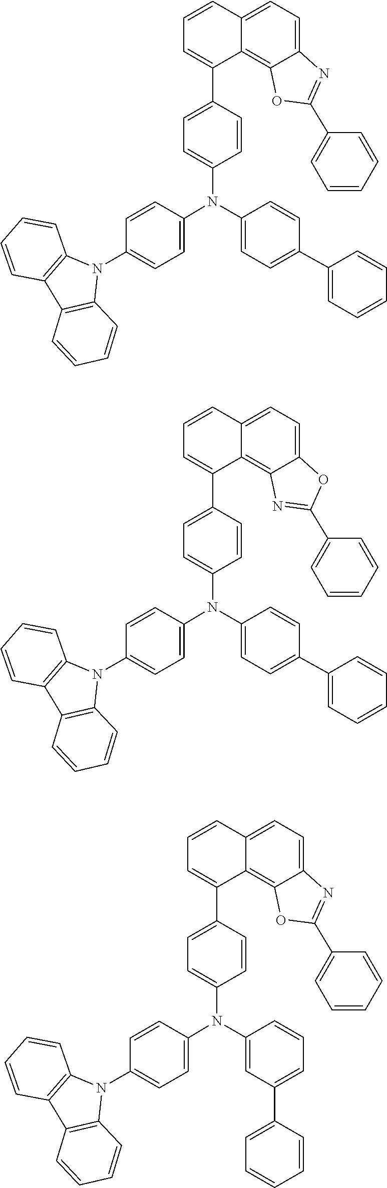 [SMILES: c1ccc(-c2ccc(N(c3ccc(-c4cccc5ccc6nc(-c7ccccc7)oc6c45)cc3)c3ccc(-n4c5ccccc5c5ccccc54)cc3)cc2)cc1 ; c1ccc(-c2ccc(N(c3ccc(-c4cccc5ccc6oc(-c7ccccc7)nc6c45)cc3)c3ccc(-n4c5ccccc5c5ccccc54)cc3)cc2)cc1 ; c1ccc(-c2cccc(N(c3ccc(-c4cccc5ccc6nc(-c7ccccc7)oc6c45)cc3)c3ccc(-n4c5ccccc5c5ccccc54)cc3)c2)cc1]

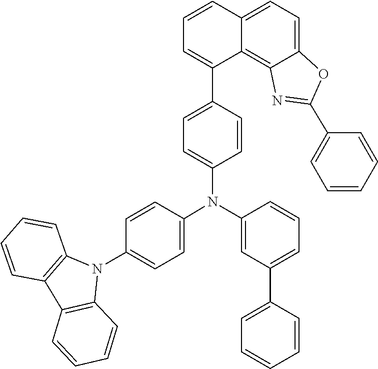 [SMILES: c1ccc(-c2cccc(N(c3ccc(-c4cccc5ccc6oc(-c7ccccc7)nc6c45)cc3)c3ccc(-n4c5ccccc5c5ccccc54)cc3)c2)cc1]

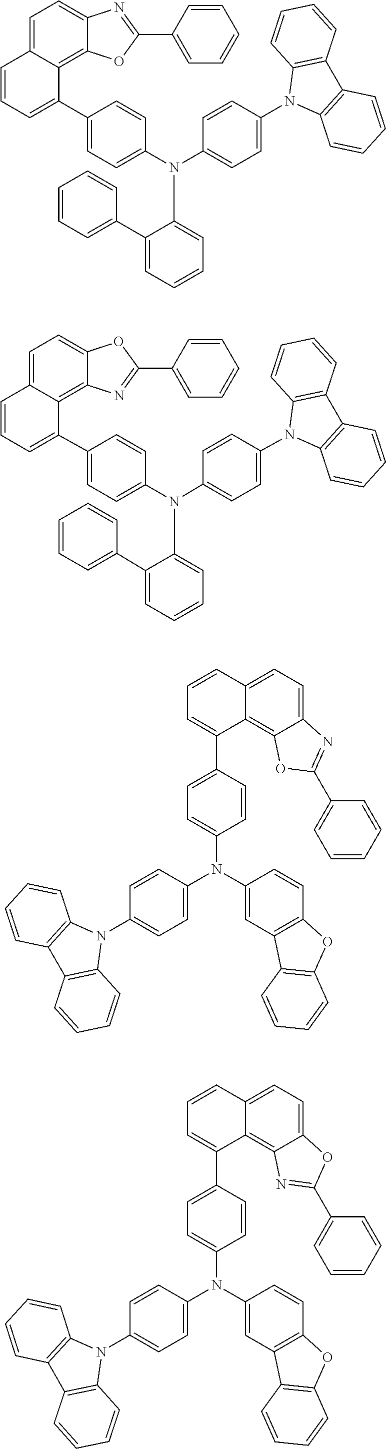 [SMILES: c1ccc(-c2nc3ccc4cccc(-c5ccc(N(c6ccc(-n7c8ccccc8c8ccccc87)cc6)c6ccccc6-c6ccccc6)cc5)c4c3o2)cc1 ; c1ccc(-c2nc3c(ccc4cccc(-c5ccc(N(c6ccc(-n7c8ccccc8c8ccccc87)cc6)c6ccccc6-c6ccccc6)cc5)c43)o2)cc1 ; c1ccc(-c2nc3ccc4cccc(-c5ccc(N(c6ccc(-n7c8ccccc8c8ccccc87)cc6)c6ccc7oc8ccccc8c7c6)cc5)c4c3o2)cc1 ; c1ccc(-c2nc3c(ccc4cccc(-c5ccc(N(c6ccc(-n7c8ccccc8c8ccccc87)cc6)c6ccc7oc8ccccc8c7c6)cc5)c43)o2)cc1]

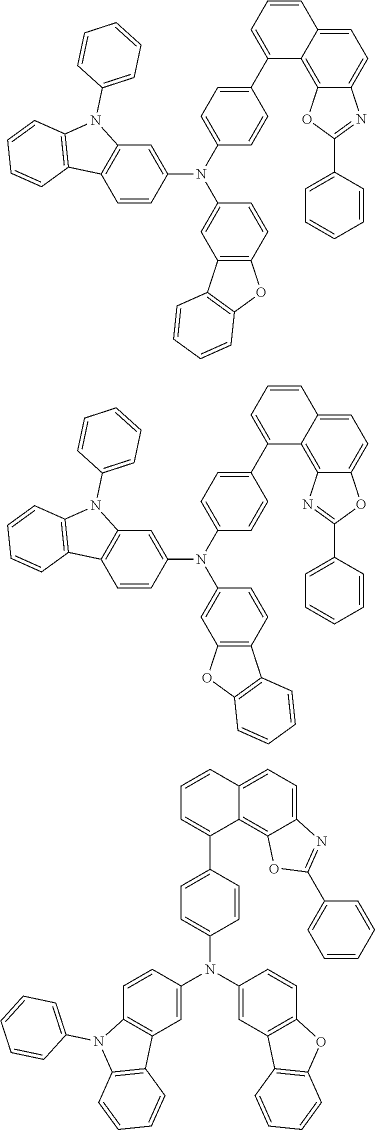

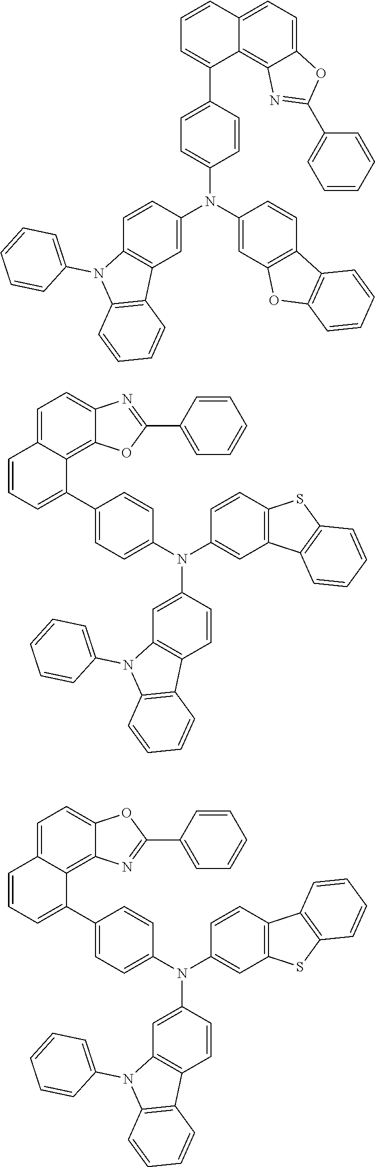

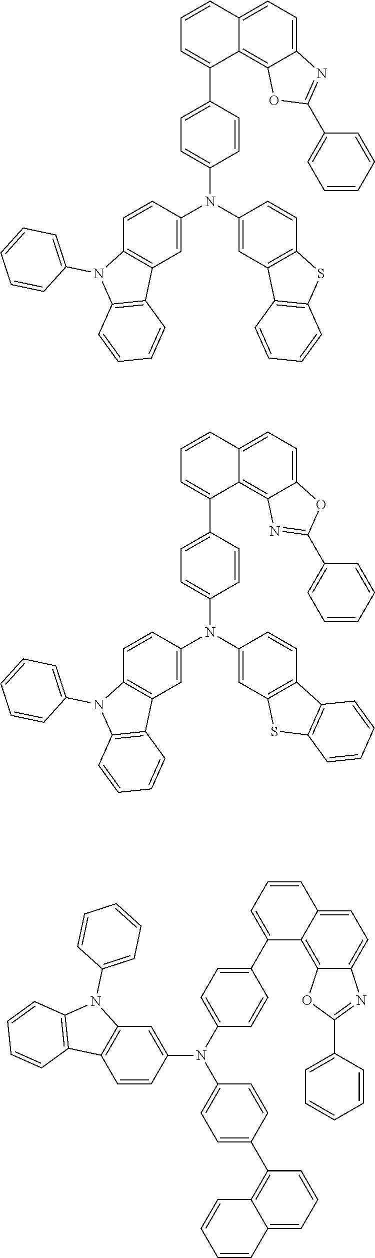 [SMILES: c1ccc(-c2nc3ccc4cccc(-c5ccc(N(c6ccc7sc8ccccc8c7c6)c6ccc7c(c6)c6ccccc6n7-c6ccccc6)cc5)c4c3o2)cc1 ; c1ccc(-c2nc3c(ccc4cccc(-c5ccc(N(c6ccc7c(c6)sc6ccccc67)c6ccc7c(c6)c6ccccc6n7-c6ccccc6)cc5)c43)o2)cc1 ; c1ccc(-c2nc3ccc4cccc(-c5ccc(N(c6ccc(-c7cccc8ccccc78)cc6)c6ccc7c8ccccc8n(-c8ccccc8)c7c6)cc5)c4c3o2)cc1]

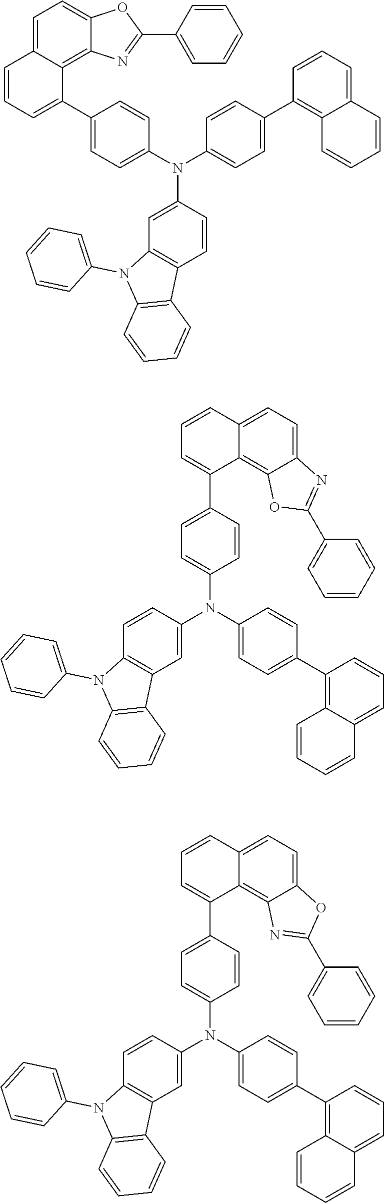 [SMILES: c1ccc(-c2nc3c(ccc4cccc(-c5ccc(N(c6ccc(-c7cccc8ccccc78)cc6)c6ccc7c8ccccc8n(-c8ccccc8)c7c6)cc5)c43)o2)cc1 ; c1ccc(-c2nc3ccc4cccc(-c5ccc(N(c6ccc(-c7cccc8ccccc78)cc6)c6ccc7c(c6)c6ccccc6n7-c6ccccc6)cc5)c4c3o2)cc1 ; c1ccc(-c2nc3c(ccc4cccc(-c5ccc(N(c6ccc(-c7cccc8ccccc78)cc6)c6ccc7c(c6)c6ccccc6n7-c6ccccc6)cc5)c43)o2)cc1]

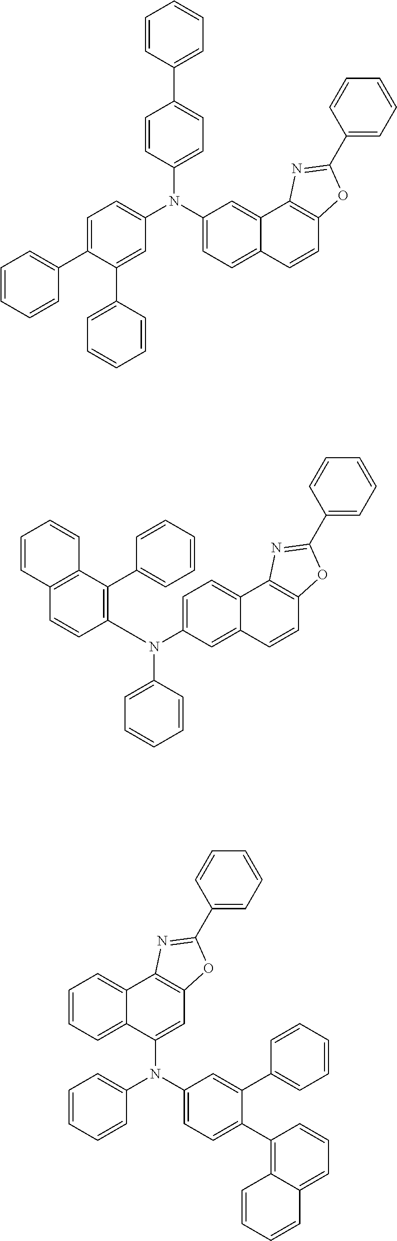 [SMILES: c1ccc(-c2ccc(N(c3ccc(-c4ccccc4)c(-c4ccccc4)c3)c3ccc4ccc5oc(-c6ccccc6)nc5c4c3)cc2)cc1 ; c1ccc(-c2nc3c(ccc4cc(N(c5ccccc5)c5ccc6ccccc6c5-c5ccccc5)ccc43)o2)cc1 ; c1ccc(-c2nc3c(cc(N(c4ccccc4)c4ccc(-c5cccc6ccccc56)c(-c5ccccc5)c4)c4ccccc43)o2)cc1]

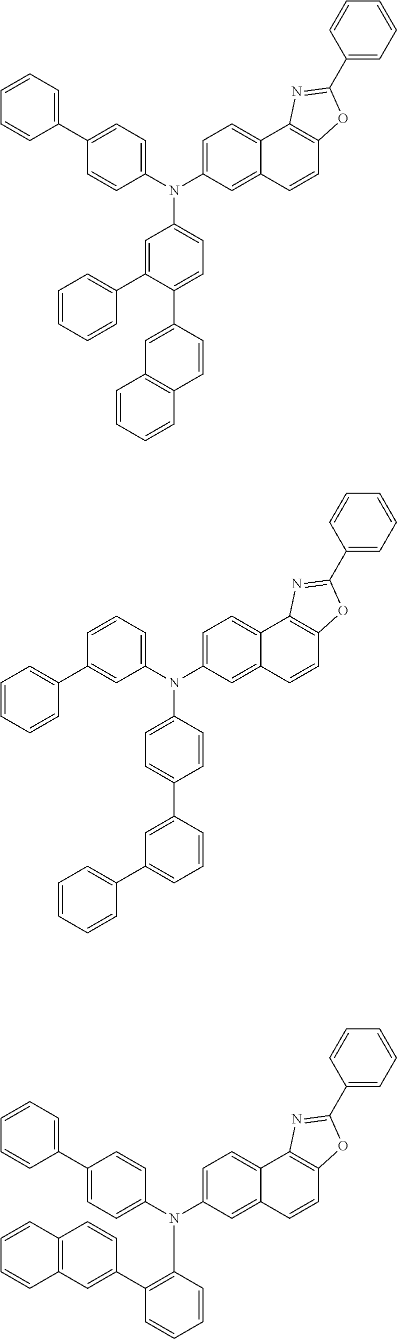 [SMILES: c1ccc(-c2ccc(N(c3ccc(-c4ccc5ccccc5c4)c(-c4ccccc4)c3)c3ccc4c(ccc5oc(-c6ccccc6)nc54)c3)cc2)cc1 ; c1ccc(-c2cccc(-c3ccc(N(c4cccc(-c5ccccc5)c4)c4ccc5c(ccc6oc(-c7ccccc7)nc65)c4)cc3)c2)cc1 ; c1ccc(-c2ccc(N(c3ccc4c(ccc5oc(-c6ccccc6)nc54)c3)c3ccccc3-c3ccc4ccccc4c3)cc2)cc1]

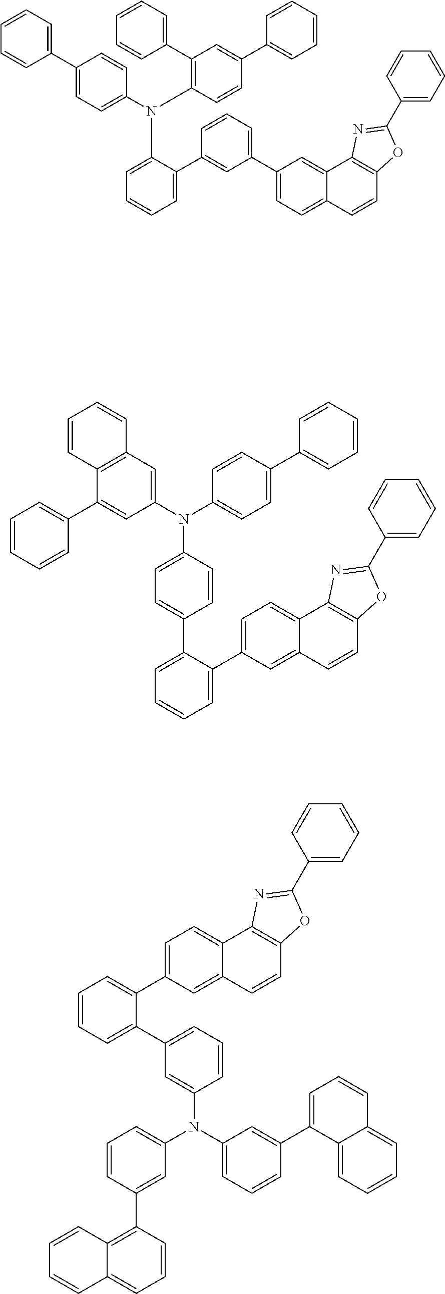 [SMILES: c1ccc(-c2ccc(N(c3ccccc3-c3cccc(-c4ccc5ccc6oc(-c7ccccc7)nc6c5c4)c3)c3ccc(-c4ccccc4)cc3-c3ccccc3)cc2)cc1 ; c1ccc(-c2ccc(N(c3ccc(-c4ccccc4-c4ccc5c(ccc6oc(-c7ccccc7)nc65)c4)cc3)c3cc(-c4ccccc4)c4ccccc4c3)cc2)cc1 ; c1ccc(-c2nc3c(ccc4cc(-c5ccccc5-c5cccc(N(c6cccc(-c7cccc8ccccc78)c6)c6cccc(-c7cccc8ccccc78)c6)c5)ccc43)o2)cc1]

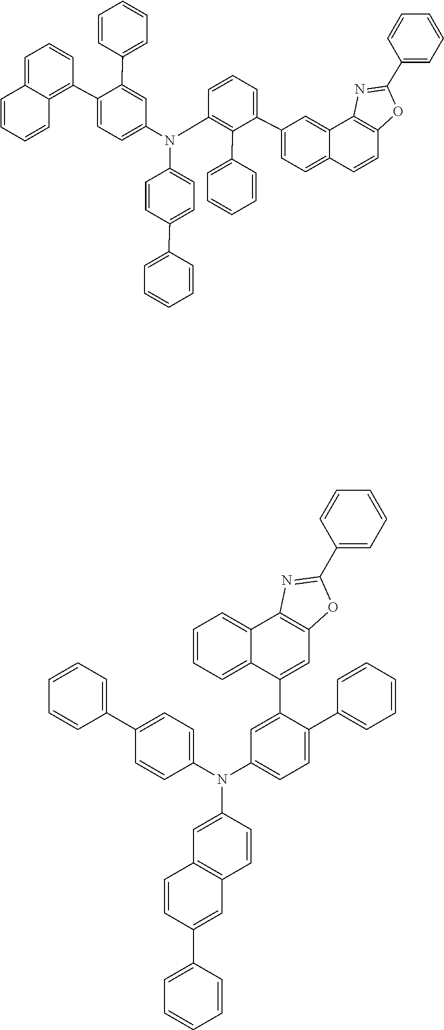 [SMILES: c1ccc(-c2ccc(N(c3ccc(-c4cccc5ccccc45)c(-c4ccccc4)c3)c3cccc(-c4ccc5ccc6oc(-c7ccccc7)nc6c5c4)c3-c3ccccc3)cc2)cc1 ; c1ccc(-c2ccc(N(c3ccc(-c4ccccc4)c(-c4cc5oc(-c6ccccc6)nc5c5ccccc45)c3)c3ccc4cc(-c5ccccc5)ccc4c3)cc2)cc1]

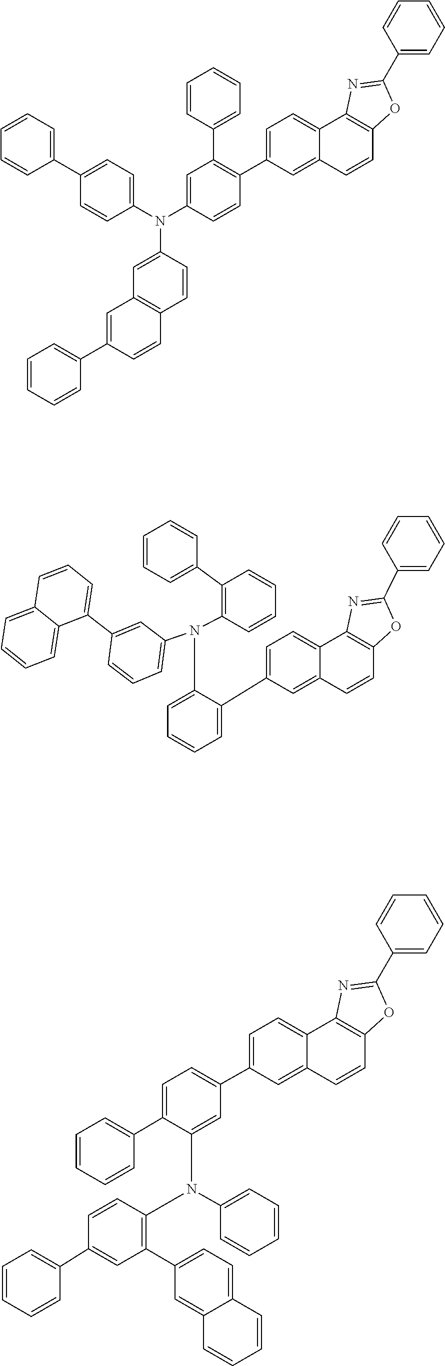 [SMILES: c1ccc(-c2ccc(N(c3ccc(-c4ccc5c(ccc6oc(-c7ccccc7)nc65)c4)c(-c4ccccc4)c3)c3ccc4ccc(-c5ccccc5)cc4c3)cc2)cc1 ; c1ccc(-c2nc3c(ccc4cc(-c5ccccc5N(c5cccc(-c6cccc7ccccc67)c5)c5ccccc5-c5ccccc5)ccc43)o2)cc1 ; c1ccc(-c2ccc(N(c3ccccc3)c3cc(-c4ccc5c(ccc6oc(-c7ccccc7)nc65)c4)ccc3-c3ccccc3)c(-c3ccc4ccccc4c3)c2)cc1]

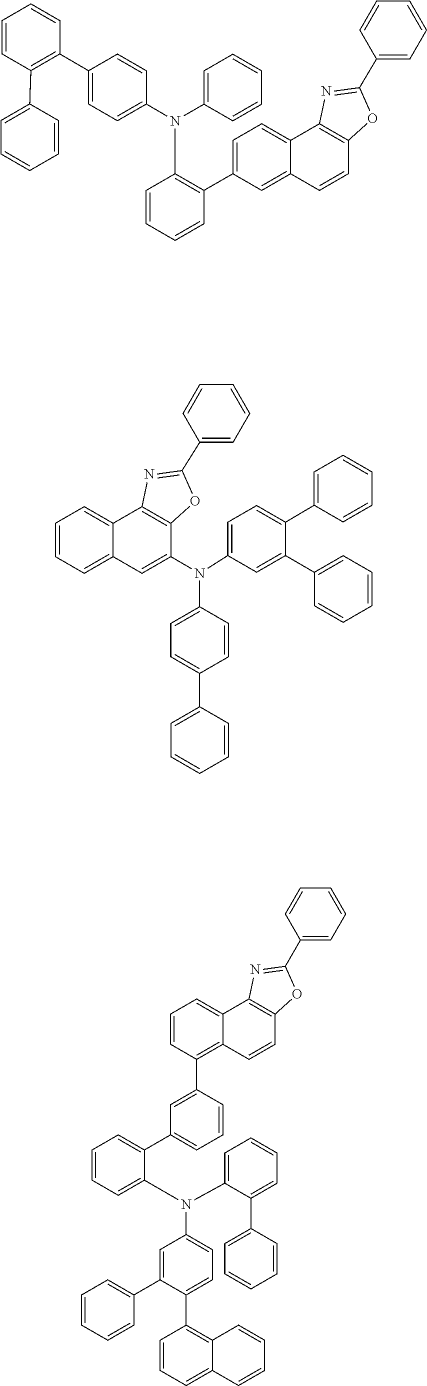 [SMILES: c1ccc(-c2nc3c(ccc4cc(-c5ccccc5N(c5ccccc5)c5ccc(-c6ccccc6-c6ccccc6)cc5)ccc43)o2)cc1 ; c1ccc(-c2ccc(N(c3ccc(-c4ccccc4)c(-c4ccccc4)c3)c3cc4ccccc4c4nc(-c5ccccc5)oc34)cc2)cc1 ; c1ccc(-c2nc3c(ccc4c(-c5cccc(-c6ccccc6N(c6ccc(-c7cccc8ccccc78)c(-c7ccccc7)c6)c6ccccc6-c6ccccc6)c5)cccc43)o2)cc1]

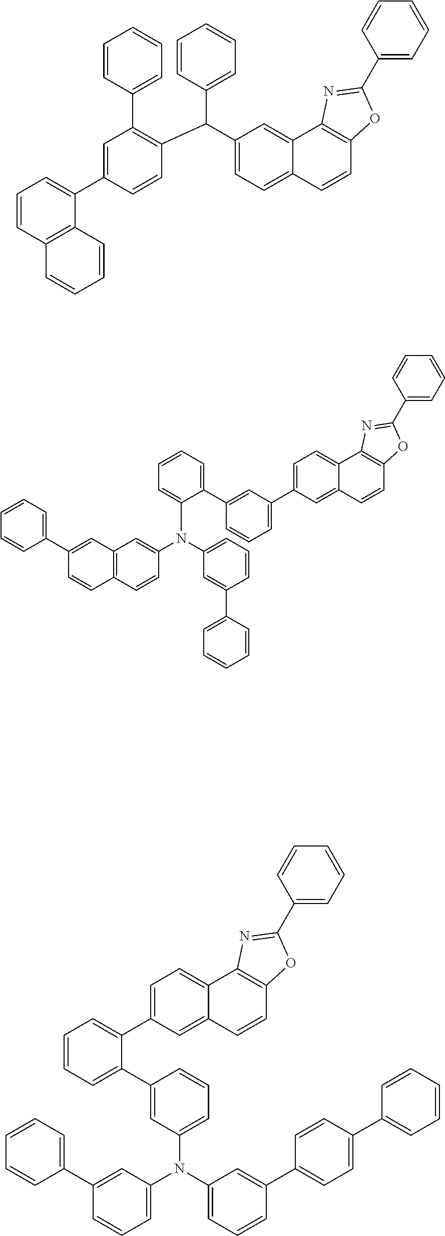 [SMILES: c1ccc(-c2nc3c(ccc4ccc(C(c5ccccc5)c5ccc(-c6cccc7ccccc67)cc5-c5ccccc5)cc43)o2)cc1 ; c1ccc(-c2cccc(N(c3ccc4ccc(-c5ccccc5)cc4c3)c3ccccc3-c3cccc(-c4ccc5c(ccc6oc(-c7ccccc7)nc65)c4)c3)c2)cc1 ; c1ccc(-c2ccc(-c3cccc(N(c4cccc(-c5ccccc5)c4)c4cccc(-c5ccccc5-c5ccc6c(ccc7oc(-c8ccccc8)nc76)c5)c4)c3)cc2)cc1]

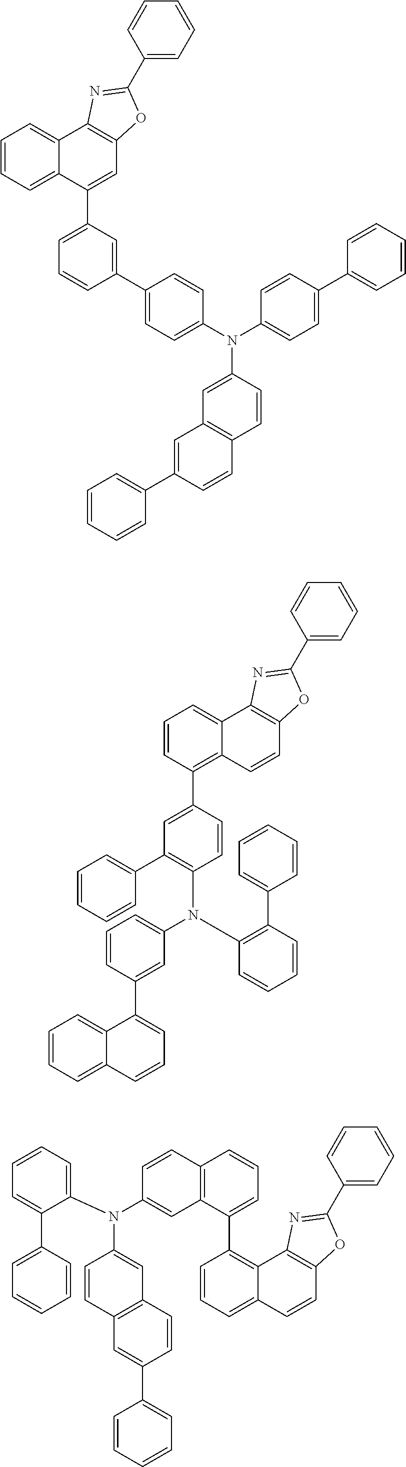 [SMILES: c1ccc(-c2ccc(N(c3ccc(-c4cccc(-c5cc6oc(-c7ccccc7)nc6c6ccccc56)c4)cc3)c3ccc4ccc(-c5ccccc5)cc4c3)cc2)cc1 ; c1ccc(-c2nc3c(ccc4c(-c5ccc(N(c6cccc(-c7cccc8ccccc78)c6)c6ccccc6-c6ccccc6)c(-c6ccccc6)c5)cccc43)o2)cc1 ; c1ccc(-c2ccc3cc(N(c4ccc5cccc(-c6cccc7ccc8oc(-c9ccccc9)nc8c67)c5c4)c4ccccc4-c4ccccc4)ccc3c2)cc1]

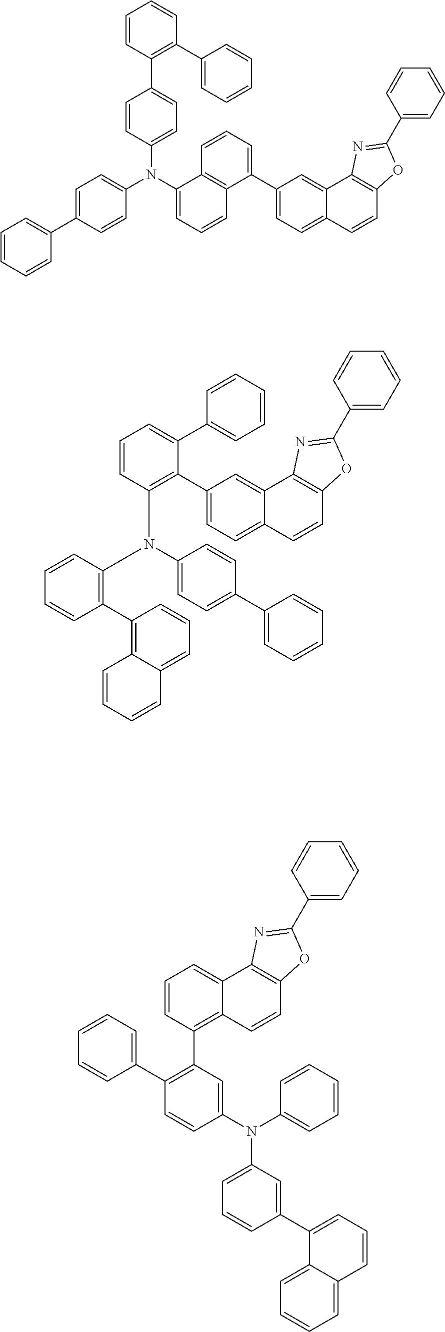 [SMILES: c1ccc(-c2ccc(N(c3ccc(-c4ccccc4-c4ccccc4)cc3)c3cccc4c(-c5ccc6ccc7oc(-c8ccccc8)nc7c6c5)cccc34)cc2)cc1 ; c1ccc(-c2ccc(N(c3ccccc3-c3cccc4ccccc34)c3cccc(-c4ccccc4)c3-c3ccc4ccc5oc(-c6ccccc6)nc5c4c3)cc2)cc1 ; c1ccc(-c2nc3c(ccc4c(-c5cc(N(c6ccccc6)c6cccc(-c7cccc8ccccc78)c6)ccc5-c5ccccc5)cccc43)o2)cc1]

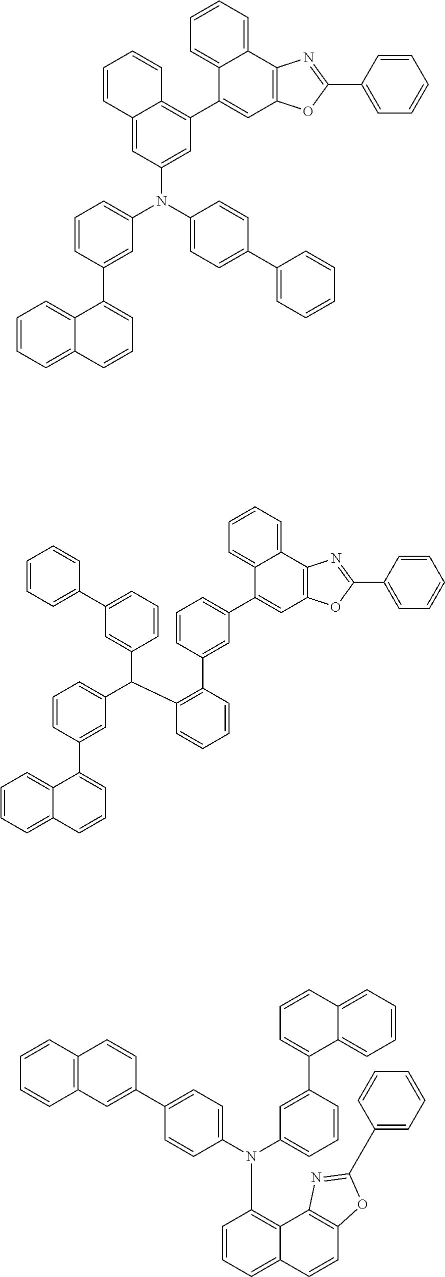 [SMILES: c1ccc(-c2ccc(N(c3cccc(-c4cccc5ccccc45)c3)c3cc(-c4cc5oc(-c6ccccc6)nc5c5ccccc45)c4ccccc4c3)cc2)cc1 ; c1ccc(-c2cccc(C(c3cccc(-c4cccc5ccccc45)c3)c3ccccc3-c3cccc(-c4cc5oc(-c6ccccc6)nc5c5ccccc45)c3)c2)cc1 ; c1ccc(-c2nc3c(ccc4cccc(N(c5ccc(-c6ccc7ccccc7c6)cc5)c5cccc(-c6cccc7ccccc67)c5)c43)o2)cc1]

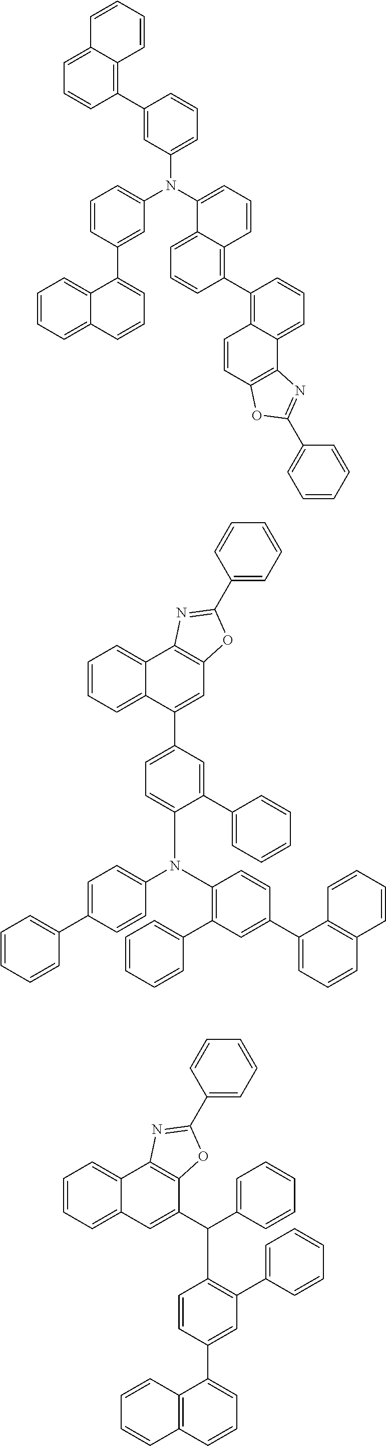

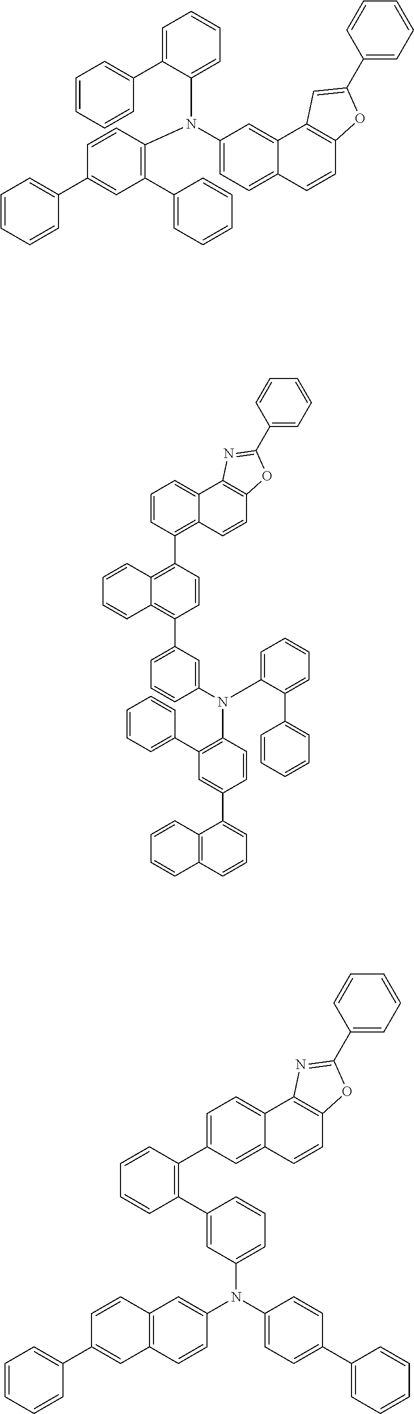 [SMILES: c1ccc(-c2ccc(N(c3ccc4ccc5oc(-c6ccccc6)cc5c4c3)c3ccccc3-c3ccccc3)c(-c3ccccc3)c2)cc1 ; c1ccc(-c2nc3c(ccc4c(-c5ccc(-c6cccc(N(c7ccccc7-c7ccccc7)c7ccc(-c8cccc9ccccc89)cc7-c7ccccc7)c6)c6ccccc56)cccc43)o2)cc1 ; c1ccc(-c2ccc(N(c3cccc(-c4ccccc4-c4ccc5c(ccc6oc(-c7ccccc7)nc65)c4)c3)c3ccc4cc(-c5ccccc5)ccc4c3)cc2)cc1]

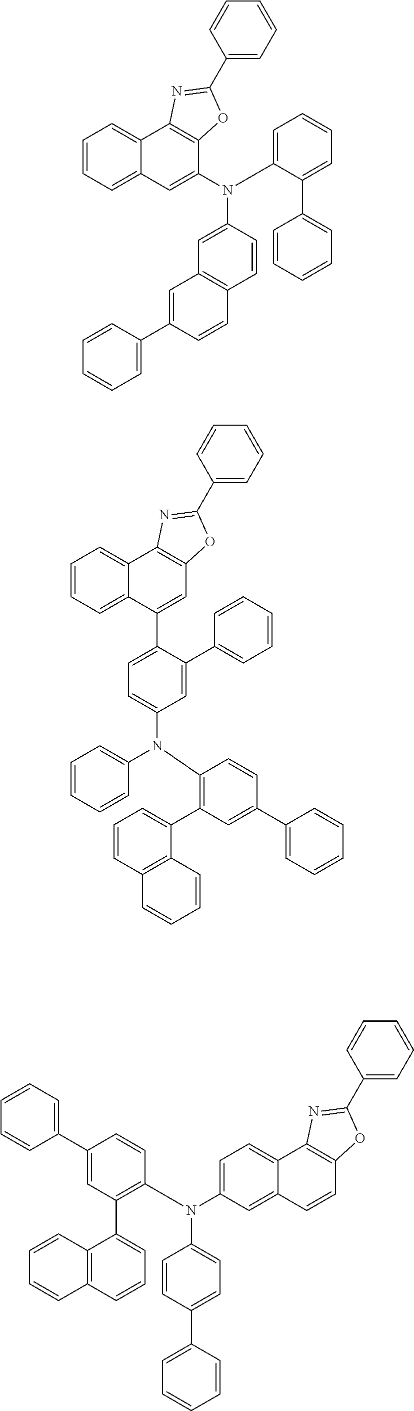 [SMILES: c1ccc(-c2ccc3ccc(N(c4ccccc4-c4ccccc4)c4cc5ccccc5c5nc(-c6ccccc6)oc45)cc3c2)cc1 ; c1ccc(-c2ccc(N(c3ccccc3)c3ccc(-c4cc5oc(-c6ccccc6)nc5c5ccccc45)c(-c4ccccc4)c3)c(-c3cccc4ccccc34)c2)cc1 ; c1ccc(-c2ccc(N(c3ccc4c(ccc5oc(-c6ccccc6)nc54)c3)c3ccc(-c4ccccc4)cc3-c3cccc4ccccc34)cc2)cc1]

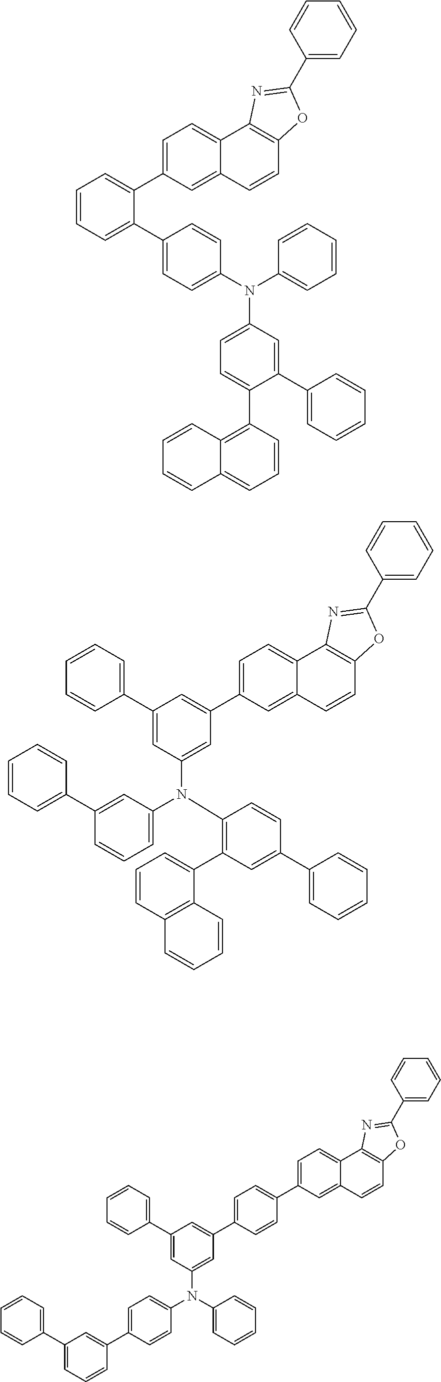 [SMILES: c1ccc(-c2nc3c(ccc4cc(-c5ccccc5-c5ccc(N(c6ccccc6)c6ccc(-c7cccc8ccccc78)c(-c7ccccc7)c6)cc5)ccc43)o2)cc1 ; c1ccc(-c2cccc(N(c3cc(-c4ccccc4)cc(-c4ccc5c(ccc6oc(-c7ccccc7)nc65)c4)c3)c3ccc(-c4ccccc4)cc3-c3cccc4ccccc34)c2)cc1 ; c1ccc(-c2cccc(-c3ccc(N(c4ccccc4)c4cc(-c5ccccc5)cc(-c5ccc(-c6ccc7c(ccc8oc(-c9ccccc9)nc87)c6)cc5)c4)cc3)c2)cc1]

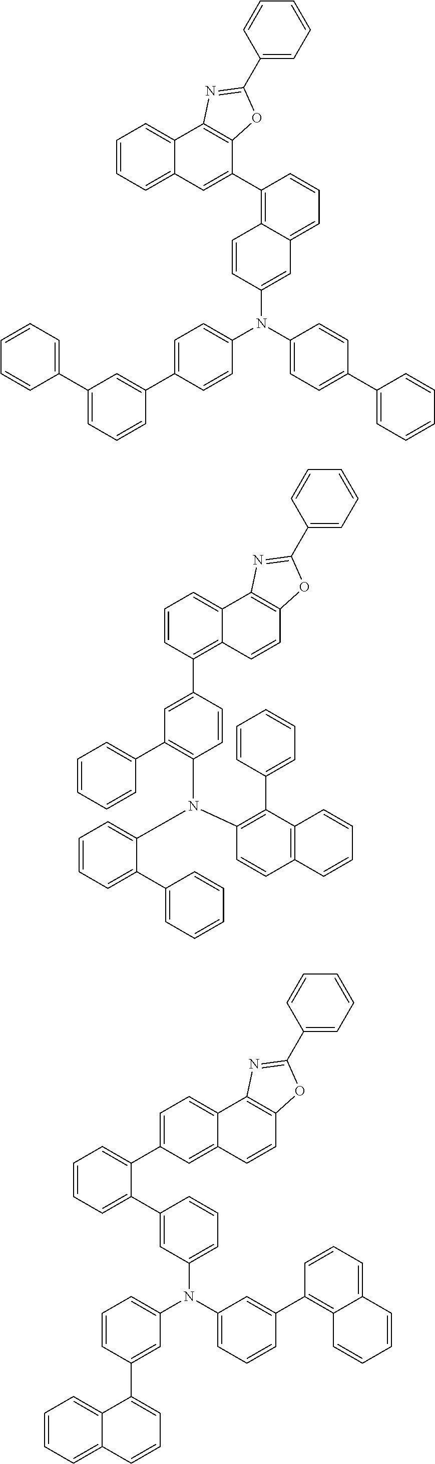 [SMILES: c1ccc(-c2ccc(N(c3ccc(-c4cccc(-c5ccccc5)c4)cc3)c3ccc4c(-c5cc6ccccc6c6nc(-c7ccccc7)oc56)cccc4c3)cc2)cc1 ; c1ccc(-c2nc3c(ccc4c(-c5ccc(N(c6ccccc6-c6ccccc6)c6ccc7ccccc7c6-c6ccccc6)c(-c6ccccc6)c5)cccc43)o2)cc1 ; c1ccc(-c2nc3c(ccc4cc(-c5ccccc5-c5cccc(N(c6cccc(-c7cccc8ccccc78)c6)c6cccc(-c7cccc8ccccc78)c6)c5)ccc43)o2)cc1]

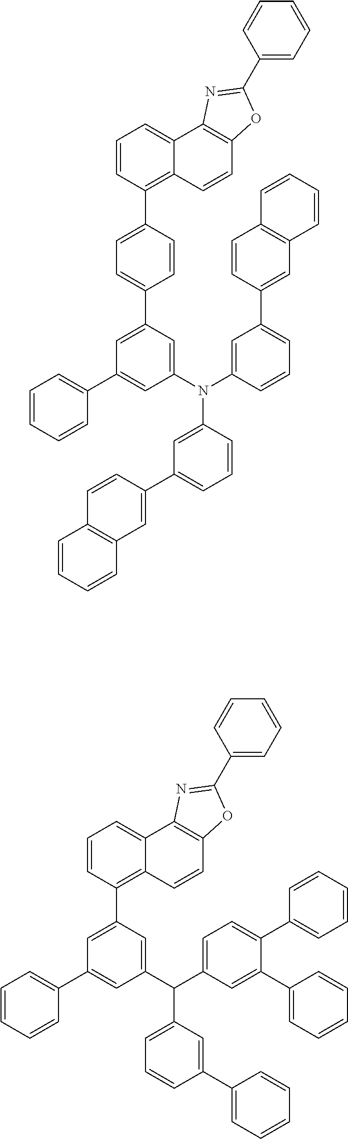 [SMILES: c1ccc(-c2cc(-c3ccc(-c4cccc5c4ccc4oc(-c6ccccc6)nc45)cc3)cc(N(c3cccc(-c4ccc5ccccc5c4)c3)c3cccc(-c4ccc5ccccc5c4)c3)c2)cc1 ; c1ccc(-c2cccc(C(c3cc(-c4ccccc4)cc(-c4cccc5c4ccc4oc(-c6ccccc6)nc45)c3)c3ccc(-c4ccccc4)c(-c4ccccc4)c3)c2)cc1]

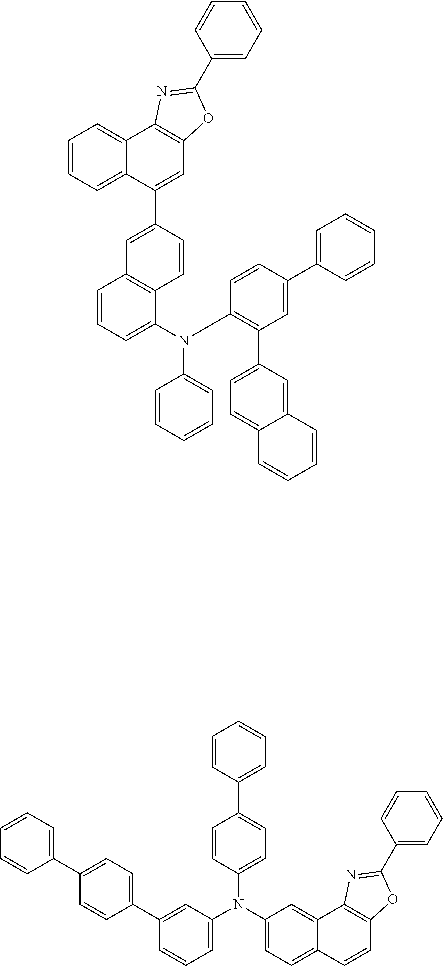 [SMILES: c1ccc(-c2ccc(N(c3ccccc3)c3cccc4cc(-c5cc6oc(-c7ccccc7)nc6c6ccccc56)ccc34)c(-c3ccc4ccccc4c3)c2)cc1 ; c1ccc(-c2ccc(-c3cccc(N(c4ccc(-c5ccccc5)cc4)c4ccc5ccc6oc(-c7ccccc7)nc6c5c4)c3)cc2)cc1]

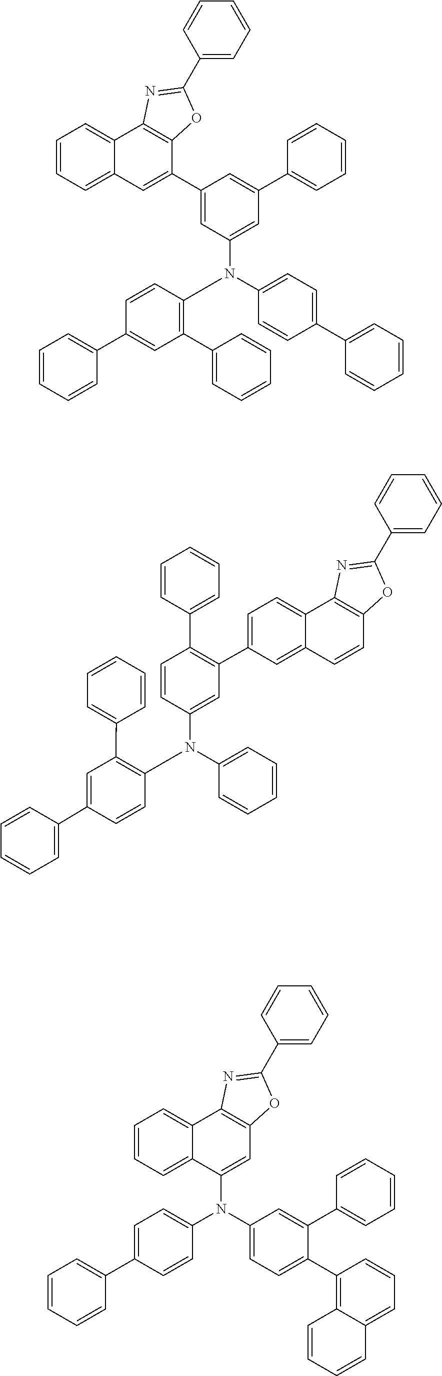 [SMILES: c1ccc(-c2ccc(N(c3cc(-c4ccccc4)cc(-c4cc5ccccc5c5nc(-c6ccccc6)oc45)c3)c3ccc(-c4ccccc4)cc3-c3ccccc3)cc2)cc1 ; c1ccc(-c2ccc(N(c3ccccc3)c3ccc(-c4ccccc4)c(-c4ccc5c(ccc6oc(-c7ccccc7)nc65)c4)c3)c(-c3ccccc3)c2)cc1 ; c1ccc(-c2ccc(N(c3ccc(-c4cccc5ccccc45)c(-c4ccccc4)c3)c3cc4oc(-c5ccccc5)nc4c4ccccc34)cc2)cc1]

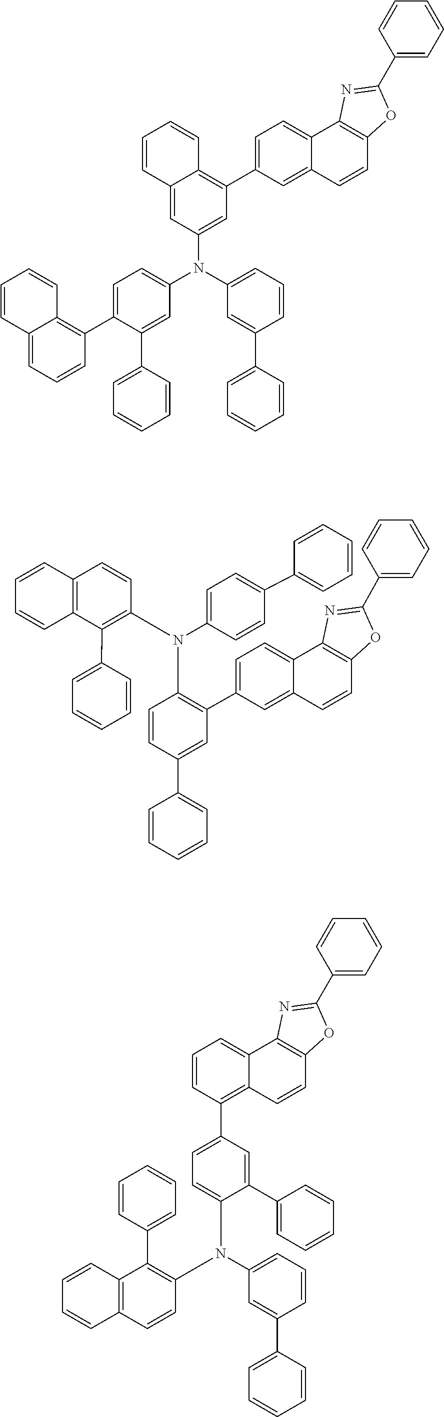 [SMILES: c1ccc(-c2cccc(N(c3ccc(-c4cccc5ccccc45)c(-c4ccccc4)c3)c3cc(-c4ccc5c(ccc6oc(-c7ccccc7)nc65)c4)c4ccccc4c3)c2)cc1 ; c1ccc(-c2ccc(N(c3ccc(-c4ccccc4)cc3-c3ccc4c(ccc5oc(-c6ccccc6)nc54)c3)c3ccc4ccccc4c3-c3ccccc3)cc2)cc1 ; c1ccc(-c2cccc(N(c3ccc(-c4cccc5c4ccc4oc(-c6ccccc6)nc45)cc3-c3ccccc3)c3ccc4ccccc4c3-c3ccccc3)c2)cc1]

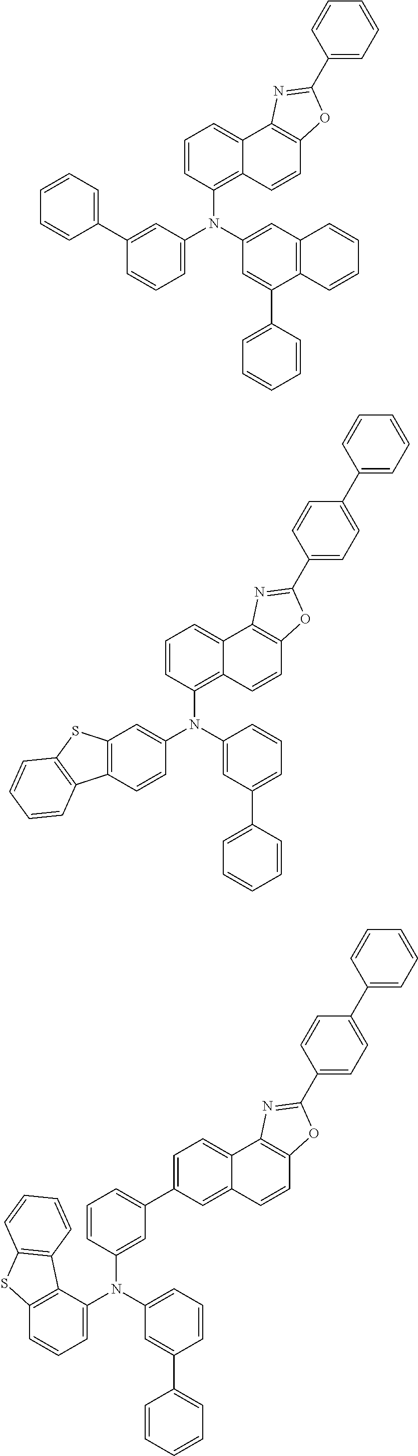 [SMILES: c1ccc(-c2cccc(N(c3cc(-c4ccccc4)c4ccccc4c3)c3cccc4c3ccc3oc(-c5ccccc5)nc34)c2)cc1 ; c1ccc(-c2ccc(-c3nc4c(ccc5c(N(c6cccc(-c7ccccc7)c6)c6ccc7c(c6)sc6ccccc67)cccc54)o3)cc2)cc1 ; c1ccc(-c2ccc(-c3nc4c(ccc5cc(-c6cccc(N(c7cccc(-c8ccccc8)c7)c7cccc8sc9ccccc9c78)c6)ccc54)o3)cc2)cc1]

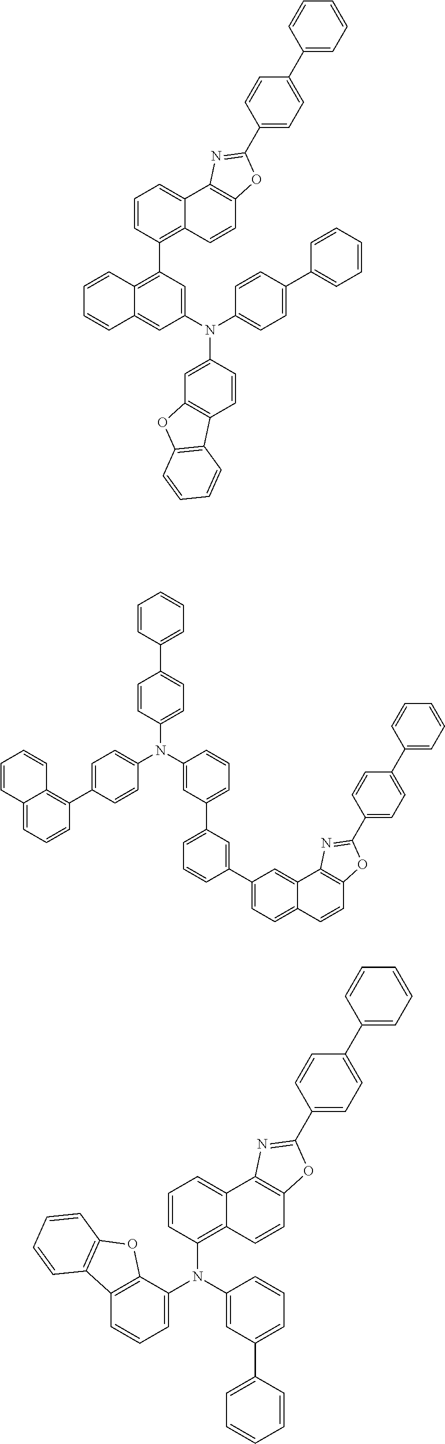 [SMILES: c1ccc(-c2ccc(-c3nc4c(ccc5c(-c6cc(N(c7ccc(-c8ccccc8)cc7)c7ccc8c(c7)oc7ccccc78)cc7ccccc67)cccc54)o3)cc2)cc1 ; c1ccc(-c2ccc(-c3nc4c(ccc5ccc(-c6cccc(-c7cccc(N(c8ccc(-c9ccccc9)cc8)c8ccc(-c9cccc%10ccccc9%10)cc8)c7)c6)cc54)o3)cc2)cc1 ; c1ccc(-c2ccc(-c3nc4c(ccc5c(N(c6cccc(-c7ccccc7)c6)c6cccc7c6oc6ccccc67)cccc54)o3)cc2)cc1]

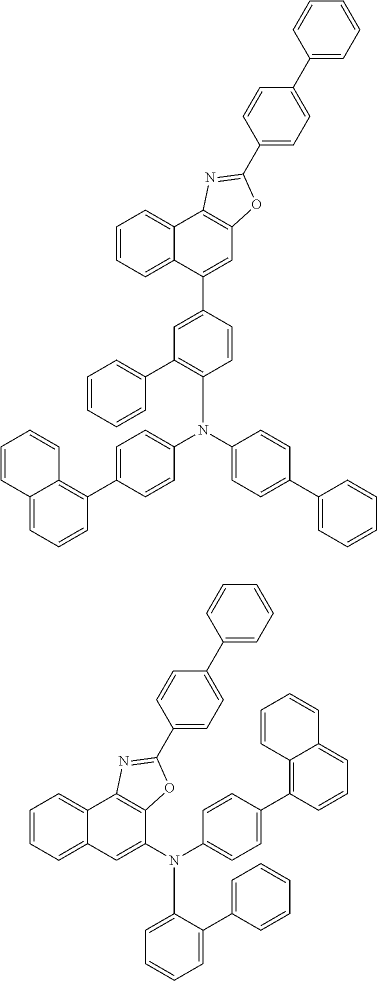 [SMILES: c1ccc(-c2ccc(-c3nc4c(cc(-c5ccc(N(c6ccc(-c7ccccc7)cc6)c6ccc(-c7cccc8ccccc78)cc6)c(-c6ccccc6)c5)c5ccccc54)o3)cc2)cc1 ; c1ccc(-c2ccc(-c3nc4c(o3)c(N(c3ccc(-c5cccc6ccccc56)cc3)c3ccccc3-c3ccccc3)cc3ccccc34)cc2)cc1]

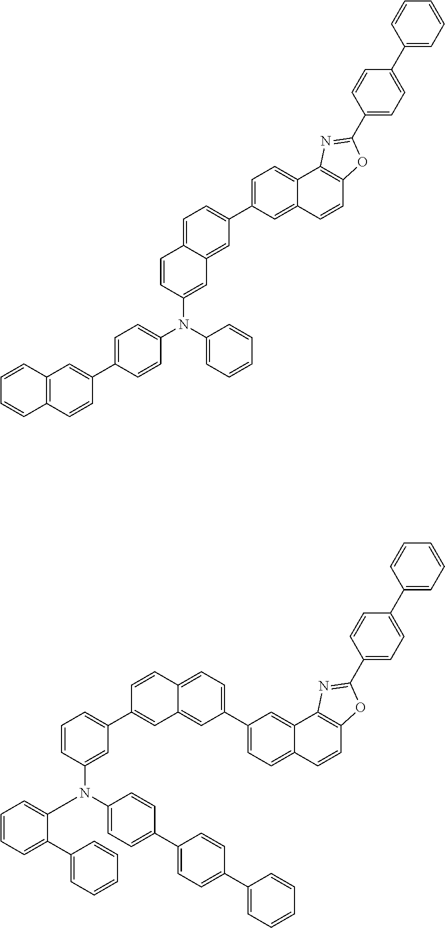 [SMILES: c1ccc(-c2ccc(-c3nc4c(ccc5cc(-c6ccc7ccc(N(c8ccccc8)c8ccc(-c9ccc%10ccccc%10c9)cc8)cc7c6)ccc54)o3)cc2)cc1 ; c1ccc(-c2ccc(-c3ccc(N(c4cccc(-c5ccc6ccc(-c7ccc8ccc9oc(-c%10ccc(-c%11ccccc%11)cc%10)nc9c8c7)cc6c5)c4)c4ccccc4-c4ccccc4)cc3)cc2)cc1]

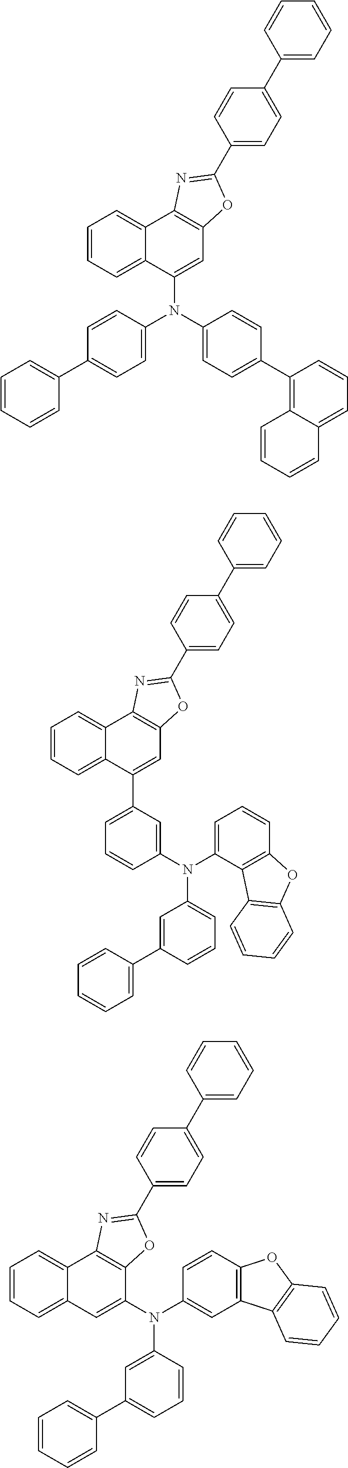

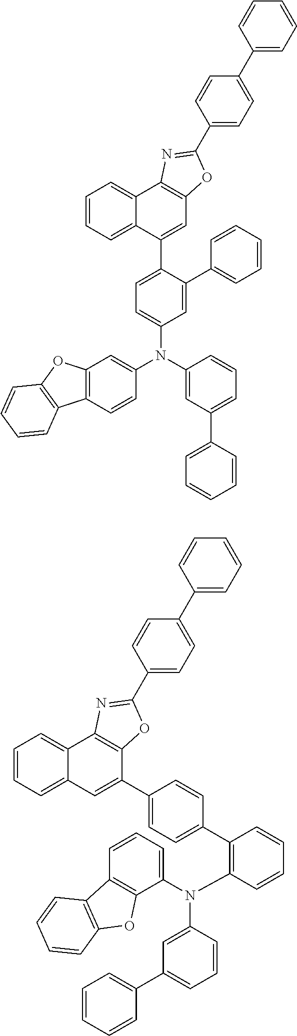

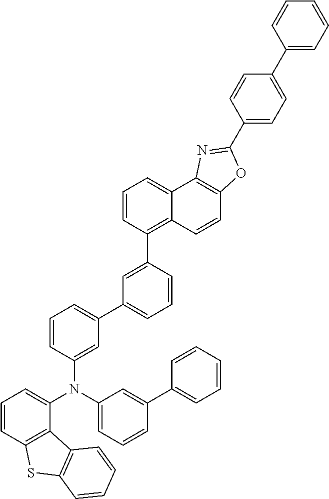 [SMILES: c1ccc(-c2ccc(-c3nc4c(ccc5c(-c6cccc(-c7cccc(N(c8cccc(-c9ccccc9)c8)c8cccc9sc%10ccccc%10c89)c7)c6)cccc54)o3)cc2)cc1]

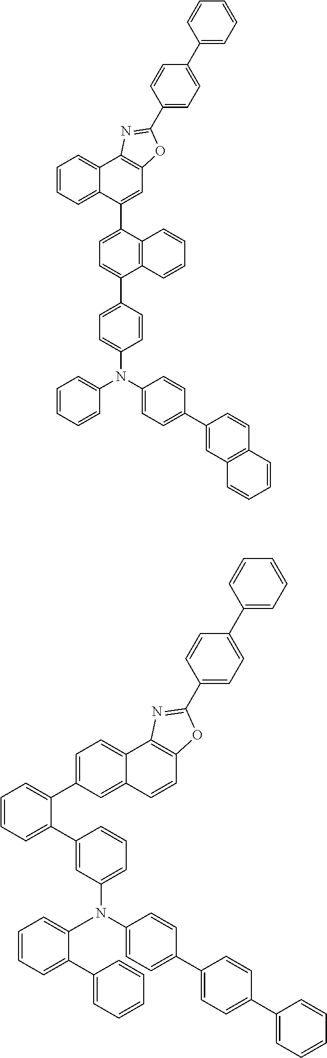 [SMILES: c1ccc(-c2ccc(-c3nc4c(cc(-c5ccc(-c6ccc(N(c7ccccc7)c7ccc(-c8ccc9ccccc9c8)cc7)cc6)c6ccccc56)c5ccccc54)o3)cc2)cc1 ; c1ccc(-c2ccc(-c3ccc(N(c4cccc(-c5ccccc5-c5ccc6c(ccc7oc(-c8ccc(-c9ccccc9)cc8)nc76)c5)c4)c4ccccc4-c4ccccc4)cc3)cc2)cc1]

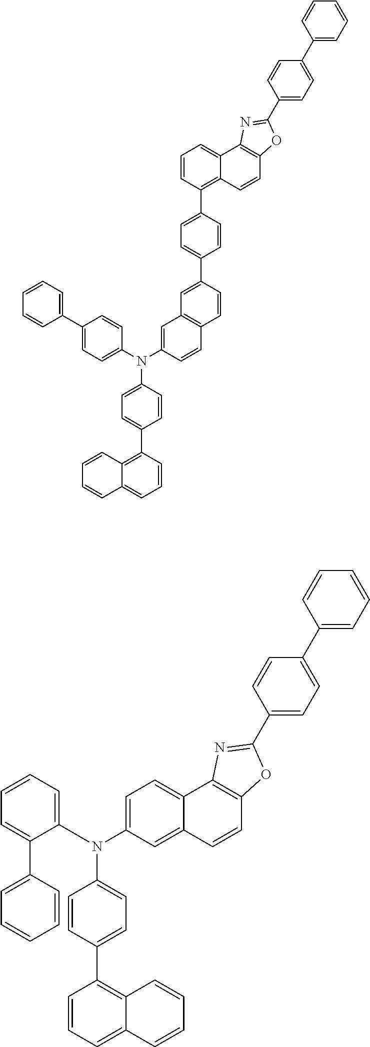 [SMILES: c1ccc(-c2ccc(-c3nc4c(ccc5c(-c6ccc(-c7ccc8ccc(N(c9ccc(-c%10ccccc%10)cc9)c9ccc(-c%10cccc%11ccccc%10%11)cc9)cc8c7)cc6)cccc54)o3)cc2)cc1 ; c1ccc(-c2ccc(-c3nc4c(ccc5cc(N(c6ccc(-c7cccc8ccccc78)cc6)c6ccccc6-c6ccccc6)ccc54)o3)cc2)cc1]

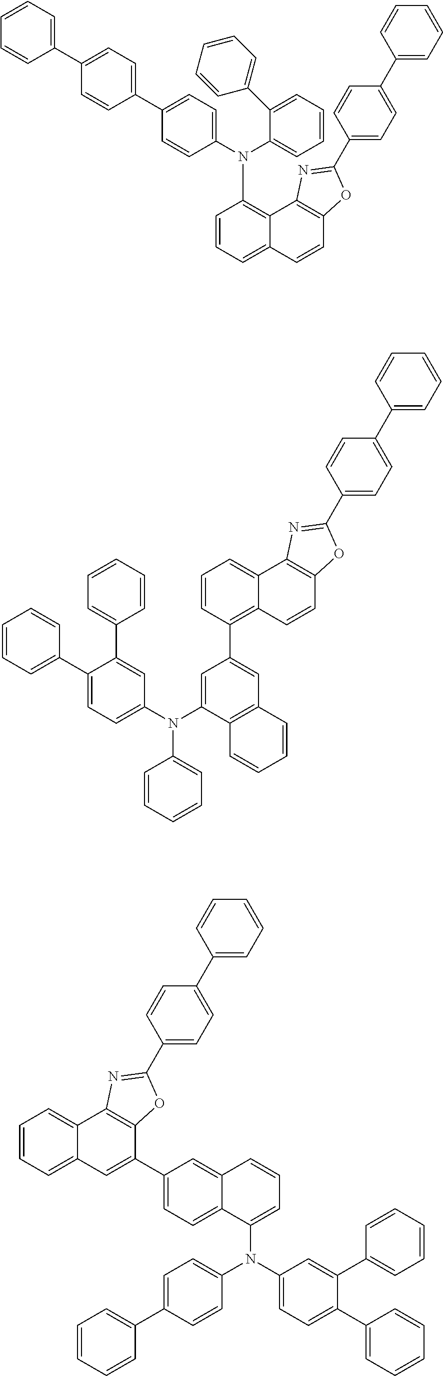 [SMILES: c1ccc(-c2ccc(-c3ccc(N(c4ccccc4-c4ccccc4)c4cccc5ccc6oc(-c7ccc(-c8ccccc8)cc7)nc6c45)cc3)cc2)cc1 ; c1ccc(-c2ccc(-c3nc4c(ccc5c(-c6cc(N(c7ccccc7)c7ccc(-c8ccccc8)c(-c8ccccc8)c7)c7ccccc7c6)cccc54)o3)cc2)cc1 ; c1ccc(-c2ccc(-c3nc4c(o3)c(-c3ccc5c(N(c6ccc(-c7ccccc7)cc6)c6ccc(-c7ccccc7)c(-c7ccccc7)c6)cccc5c3)cc3ccccc34)cc2)cc1]

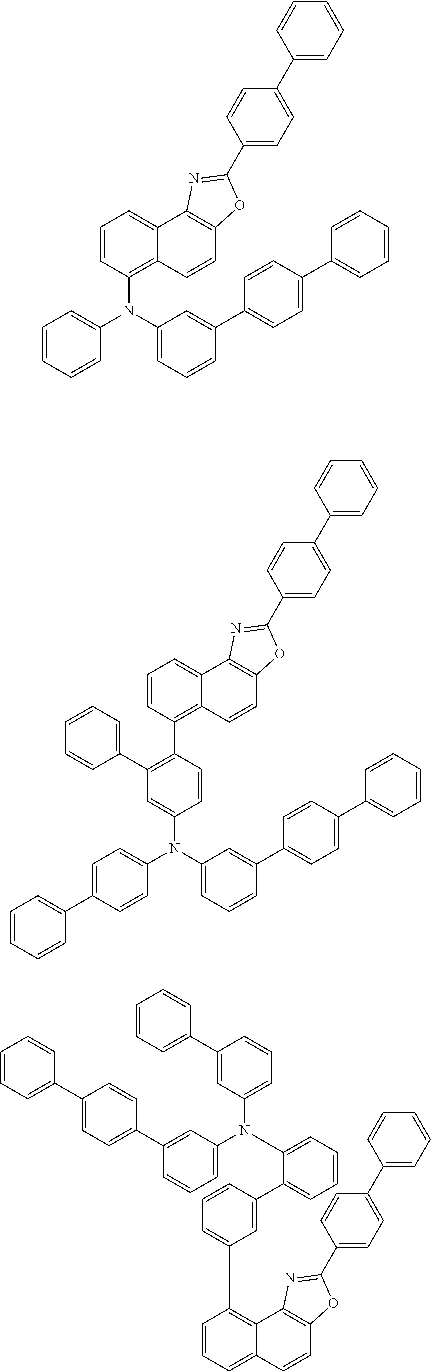 [SMILES: c1ccc(-c2ccc(-c3cccc(N(c4ccccc4)c4cccc5c4ccc4oc(-c6ccc(-c7ccccc7)cc6)nc45)c3)cc2)cc1 ; c1ccc(-c2ccc(-c3cccc(N(c4ccc(-c5ccccc5)cc4)c4ccc(-c5cccc6c5ccc5oc(-c7ccc(-c8ccccc8)cc7)nc56)c(-c5ccccc5)c4)c3)cc2)cc1 ; c1ccc(-c2ccc(-c3cccc(N(c4cccc(-c5ccccc5)c4)c4ccccc4-c4cccc(-c5cccc6ccc7oc(-c8ccc(-c9ccccc9)cc8)nc7c56)c4)c3)cc2)cc1]

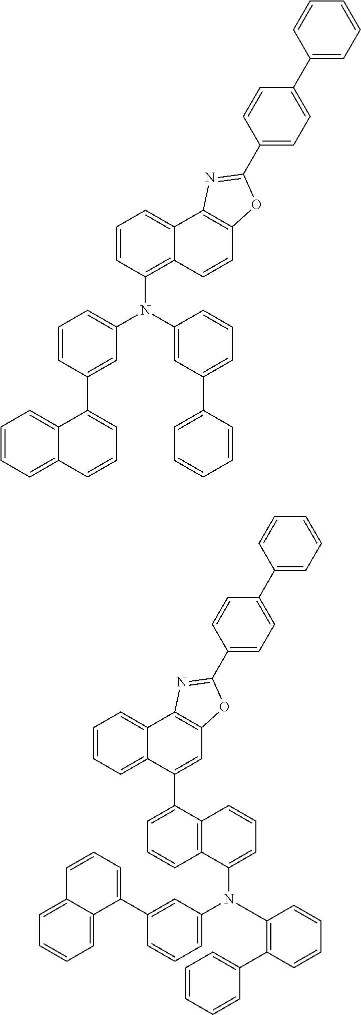 [SMILES: c1ccc(-c2ccc(-c3nc4c(ccc5c(N(c6cccc(-c7ccccc7)c6)c6cccc(-c7cccc8ccccc78)c6)cccc54)o3)cc2)cc1 ; c1ccc(-c2ccc(-c3nc4c(cc(-c5cccc6c(N(c7cccc(-c8cccc9ccccc89)c7)c7ccccc7-c7ccccc7)cccc56)c5ccccc54)o3)cc2)cc1]

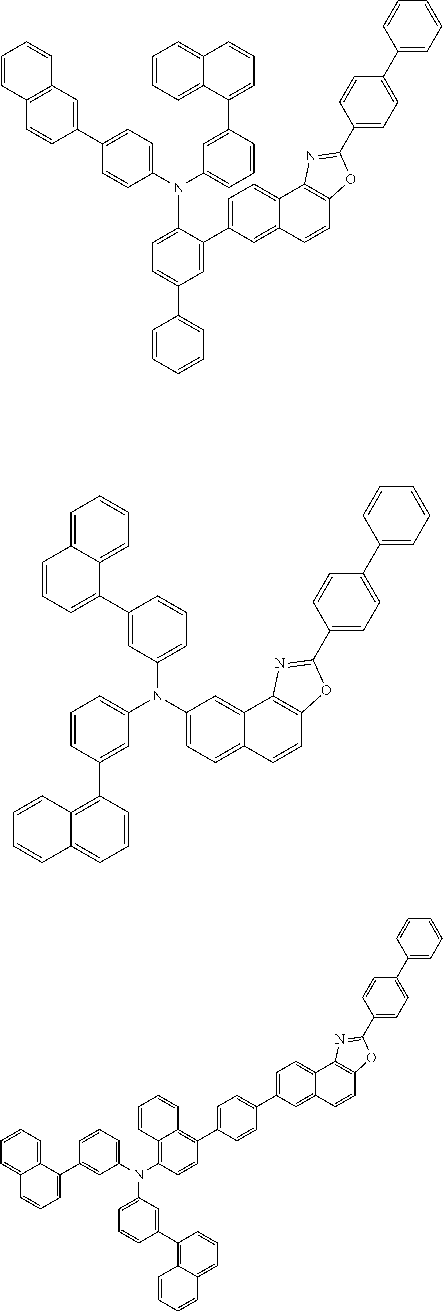 [SMILES: c1ccc(-c2ccc(-c3nc4c(ccc5cc(-c6cc(-c7ccccc7)ccc6N(c6ccc(-c7ccc8ccccc8c7)cc6)c6cccc(-c7cccc8ccccc78)c6)ccc54)o3)cc2)cc1 ; c1ccc(-c2ccc(-c3nc4c(ccc5ccc(N(c6cccc(-c7cccc8ccccc78)c6)c6cccc(-c7cccc8ccccc78)c6)cc54)o3)cc2)cc1 ; c1ccc(-c2ccc(-c3nc4c(ccc5cc(-c6ccc(-c7ccc(N(c8cccc(-c9cccc%10ccccc9%10)c8)c8cccc(-c9cccc%10ccccc9%10)c8)c8ccccc78)cc6)ccc54)o3)cc2)cc1]

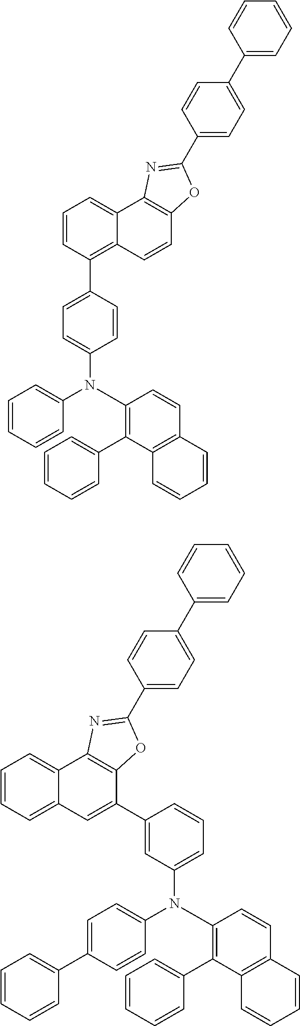 [SMILES: c1ccc(-c2ccc(-c3nc4c(ccc5c(-c6ccc(N(c7ccccc7)c7ccc8ccccc8c7-c7ccccc7)cc6)cccc54)o3)cc2)cc1 ; c1ccc(-c2ccc(-c3nc4c(o3)c(-c3cccc(N(c5ccc(-c6ccccc6)cc5)c5ccc6ccccc6c5-c5ccccc5)c3)cc3ccccc34)cc2)cc1]

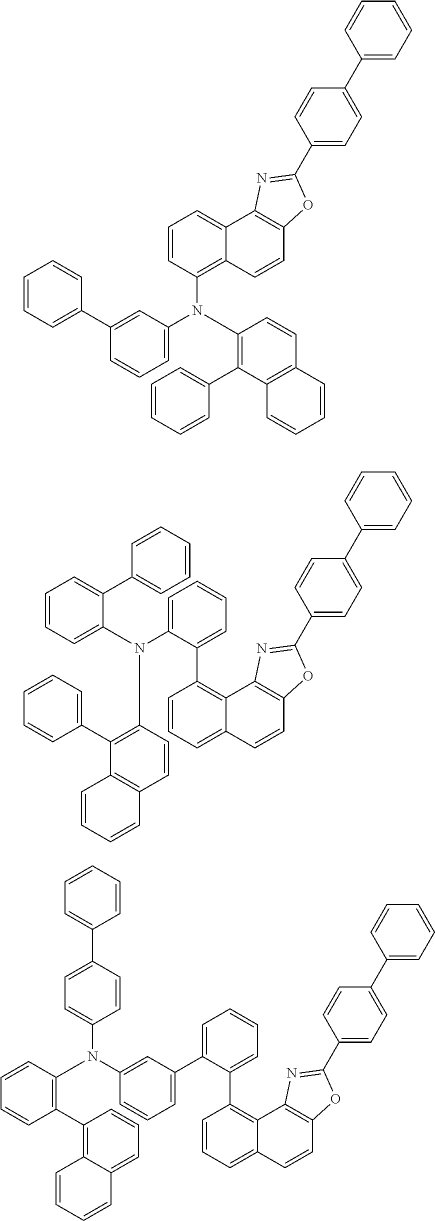

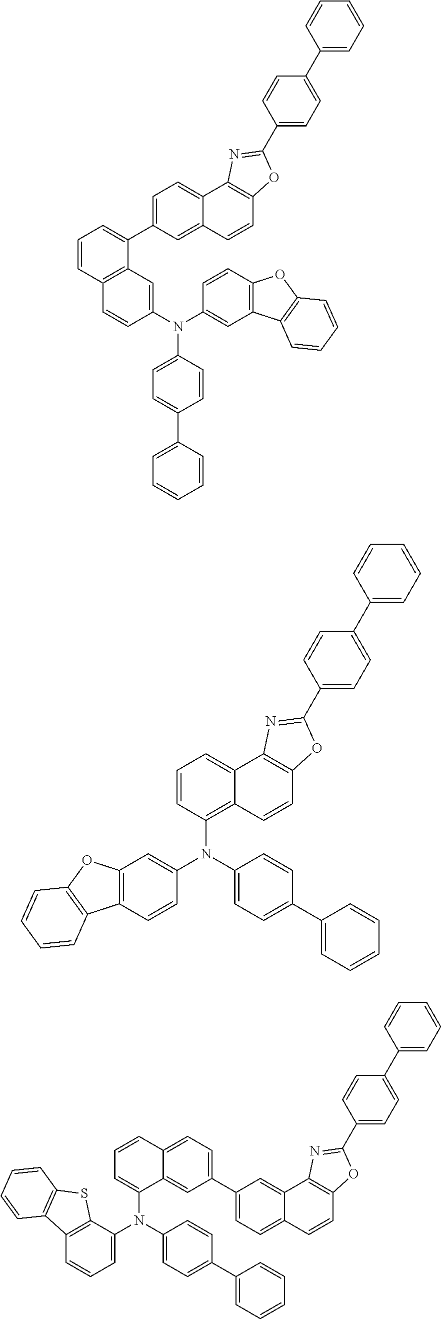 [SMILES: c1ccc(-c2ccc(-c3nc4c(ccc5cc(-c6cccc7ccc(N(c8ccc(-c9ccccc9)cc8)c8ccc9oc%10ccccc%10c9c8)cc67)ccc54)o3)cc2)cc1 ; c1ccc(-c2ccc(-c3nc4c(ccc5c(N(c6ccc(-c7ccccc7)cc6)c6ccc7c(c6)oc6ccccc67)cccc54)o3)cc2)cc1 ; c1ccc(-c2ccc(-c3nc4c(ccc5ccc(-c6ccc7cccc(N(c8ccc(-c9ccccc9)cc8)c8cccc9c8sc8ccccc89)c7c6)cc54)o3)cc2)cc1]

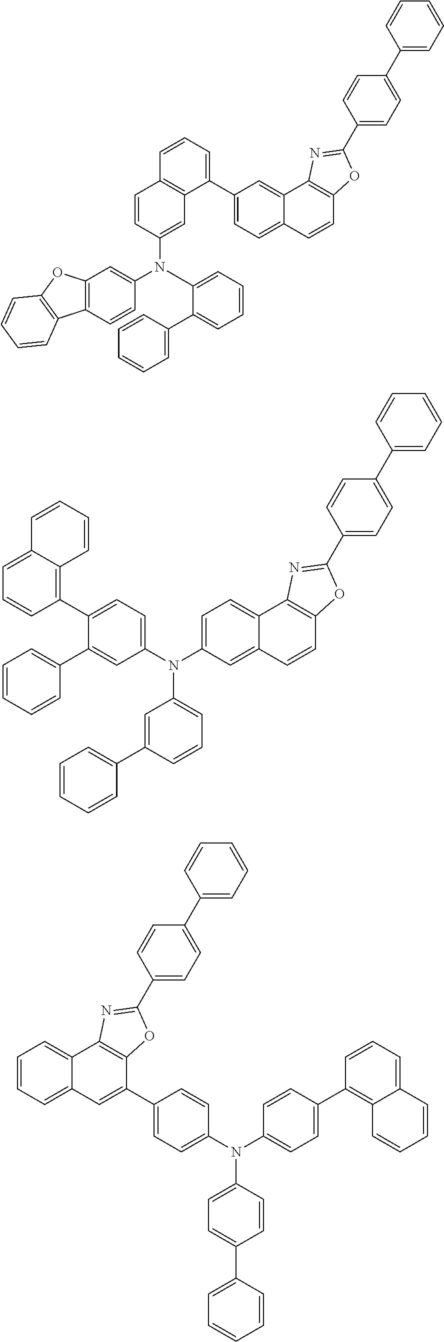 [SMILES: c1ccc(-c2ccc(-c3nc4c(ccc5ccc(-c6cccc7ccc(N(c8ccc9c(c8)oc8ccccc89)c8ccccc8-c8ccccc8)cc67)cc54)o3)cc2)cc1 ; c1ccc(-c2ccc(-c3nc4c(ccc5cc(N(c6cccc(-c7ccccc7)c6)c6ccc(-c7cccc8ccccc78)c(-c7ccccc7)c6)ccc54)o3)cc2)cc1 ; c1ccc(-c2ccc(-c3nc4c(o3)c(-c3ccc(N(c5ccc(-c6ccccc6)cc5)c5ccc(-c6cccc7ccccc67)cc5)cc3)cc3ccccc34)cc2)cc1]

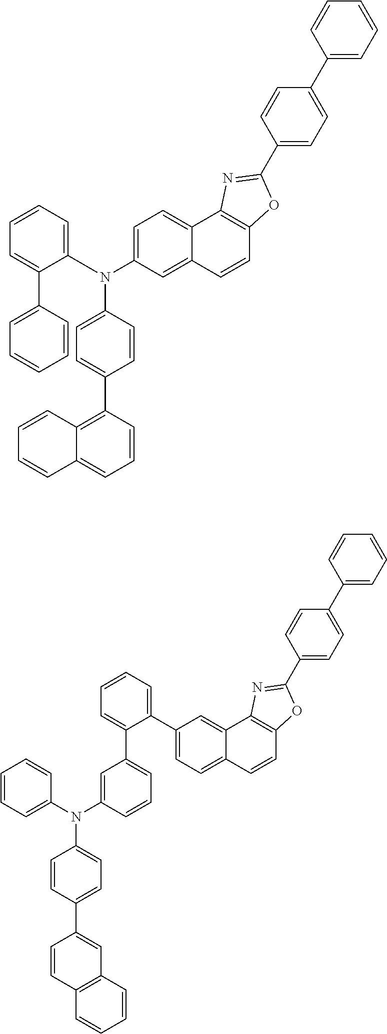 [SMILES: c1ccc(-c2ccc(-c3nc4c(ccc5cc(N(c6ccc(-c7cccc8ccccc78)cc6)c6ccccc6-c6ccccc6)ccc54)o3)cc2)cc1 ; c1ccc(-c2ccc(-c3nc4c(ccc5ccc(-c6ccccc6-c6cccc(N(c7ccccc7)c7ccc(-c8ccc9ccccc9c8)cc7)c6)cc54)o3)cc2)cc1]

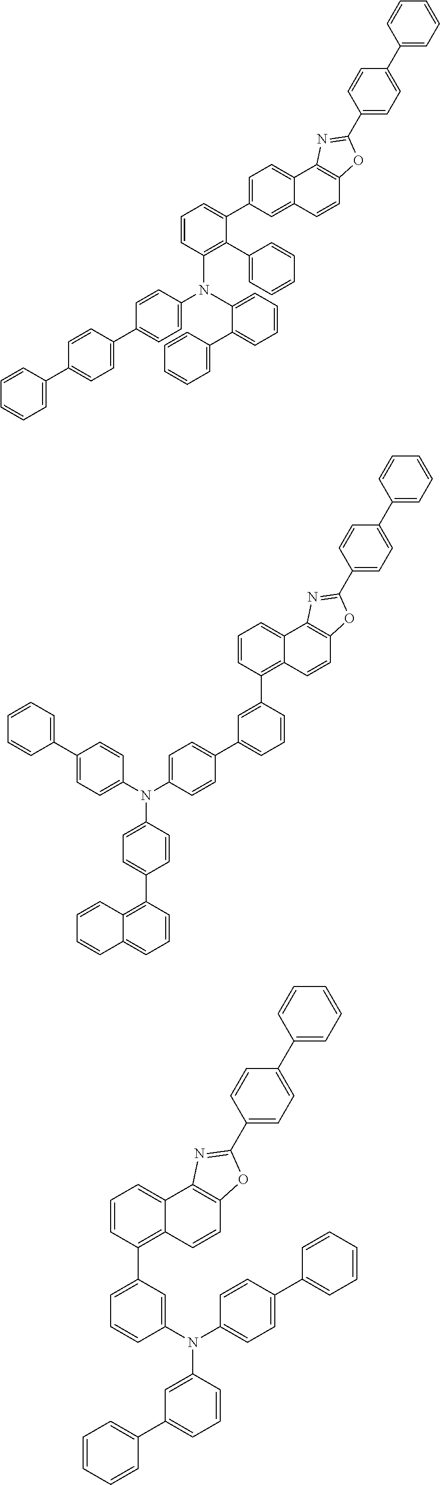 [SMILES: c1ccc(-c2ccc(-c3ccc(N(c4ccccc4-c4ccccc4)c4cccc(-c5ccc6c(ccc7oc(-c8ccc(-c9ccccc9)cc8)nc76)c5)c4-c4ccccc4)cc3)cc2)cc1 ; c1ccc(-c2ccc(-c3nc4c(ccc5c(-c6cccc(-c7ccc(N(c8ccc(-c9ccccc9)cc8)c8ccc(-c9cccc%10ccccc9%10)cc8)cc7)c6)cccc54)o3)cc2)cc1 ; c1ccc(-c2ccc(-c3nc4c(ccc5c(-c6cccc(N(c7ccc(-c8ccccc8)cc7)c7cccc(-c8ccccc8)c7)c6)cccc54)o3)cc2)cc1]

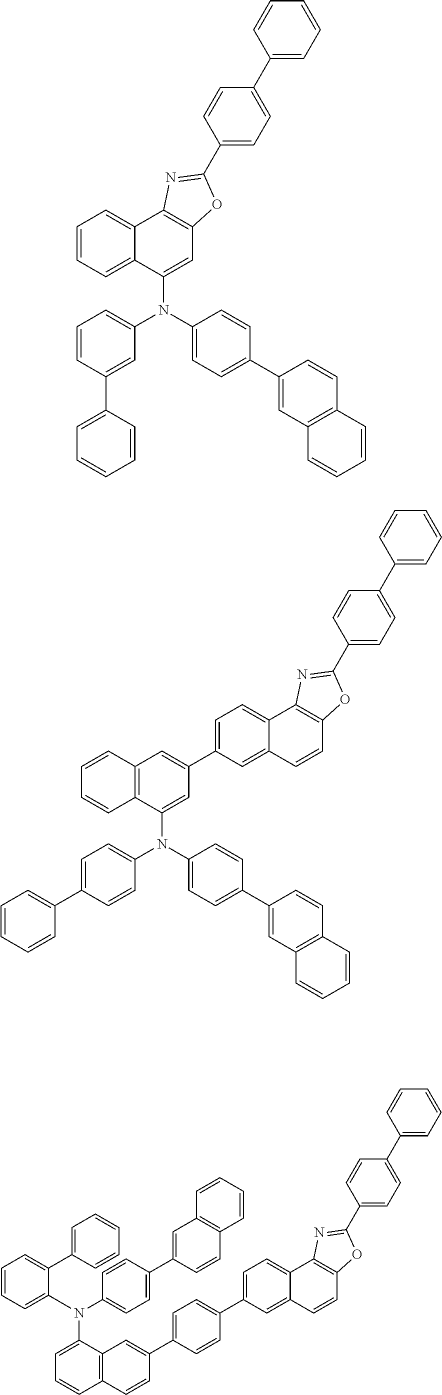 [SMILES: c1ccc(-c2ccc(-c3nc4c(cc(N(c5ccc(-c6ccc7ccccc7c6)cc5)c5cccc(-c6ccccc6)c5)c5ccccc54)o3)cc2)cc1 ; c1ccc(-c2ccc(-c3nc4c(ccc5cc(-c6cc(N(c7ccc(-c8ccccc8)cc7)c7ccc(-c8ccc9ccccc9c8)cc7)c7ccccc7c6)ccc54)o3)cc2)cc1 ; c1ccc(-c2ccc(-c3nc4c(ccc5cc(-c6ccc(-c7ccc8cccc(N(c9ccc(-c%10ccc%11ccccc%11c%10)cc9)c9ccccc9-c9ccccc9)c8c7)cc6)ccc54)o3)cc2)cc1]

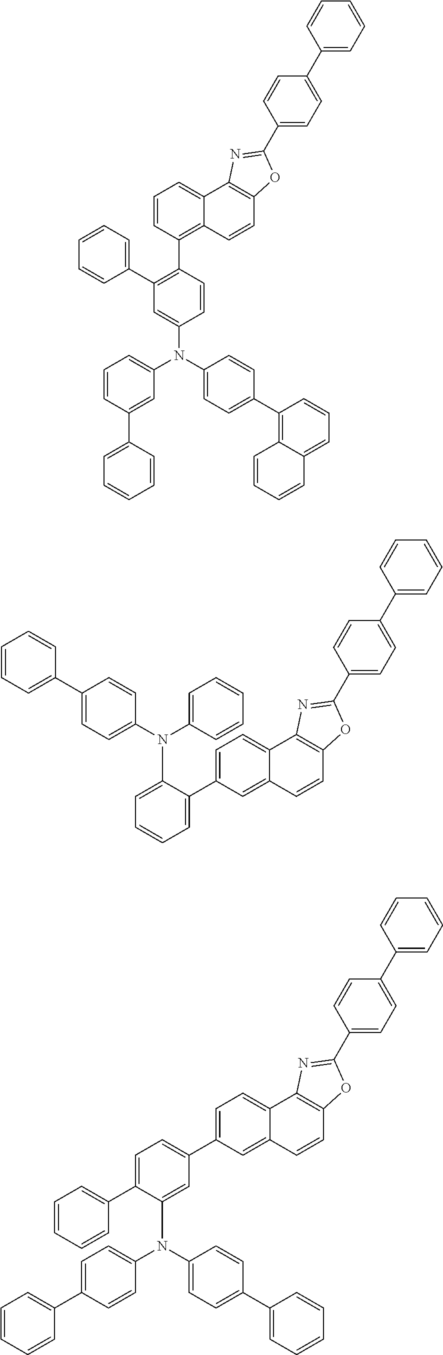 [SMILES: c1ccc(-c2ccc(-c3nc4c(ccc5c(-c6ccc(N(c7ccc(-c8cccc9ccccc89)cc7)c7cccc(-c8ccccc8)c7)cc6-c6ccccc6)cccc54)o3)cc2)cc1 ; c1ccc(-c2ccc(-c3nc4c(ccc5cc(-c6ccccc6N(c6ccccc6)c6ccc(-c7ccccc7)cc6)ccc54)o3)cc2)cc1 ; c1ccc(-c2ccc(-c3nc4c(ccc5cc(-c6ccc(-c7ccccc7)c(N(c7ccc(-c8ccccc8)cc7)c7ccc(-c8ccccc8)cc7)c6)ccc54)o3)cc2)cc1]

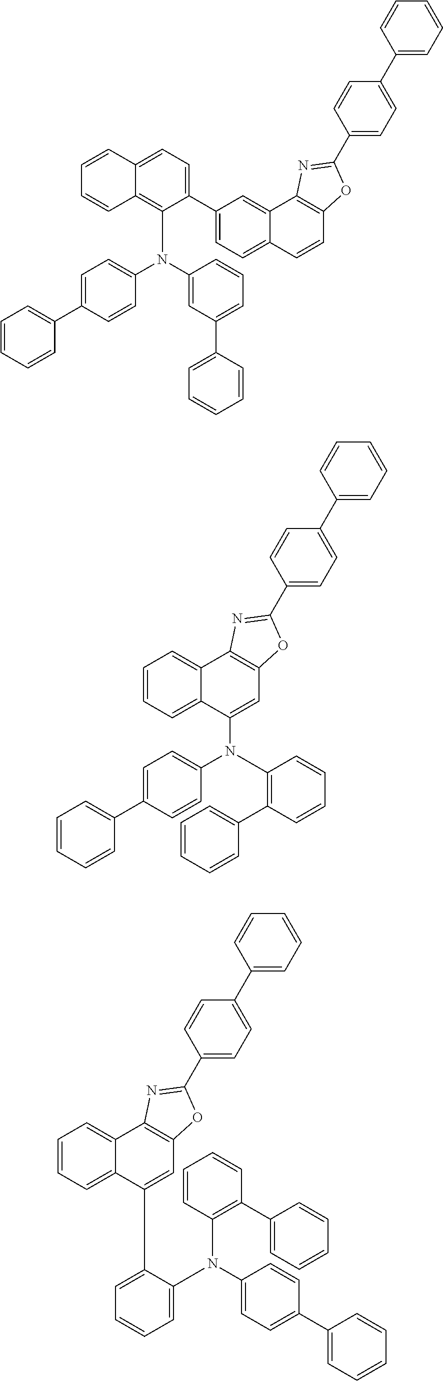 [SMILES: c1ccc(-c2ccc(-c3nc4c(ccc5ccc(-c6ccc7ccccc7c6N(c6ccc(-c7ccccc7)cc6)c6cccc(-c7ccccc7)c6)cc54)o3)cc2)cc1 ; c1ccc(-c2ccc(-c3nc4c(cc(N(c5ccc(-c6ccccc6)cc5)c5ccccc5-c5ccccc5)c5ccccc54)o3)cc2)cc1 ; c1ccc(-c2ccc(-c3nc4c(cc(-c5ccccc5N(c5ccc(-c6ccccc6)cc5)c5ccccc5-c5ccccc5)c5ccccc54)o3)cc2)cc1]

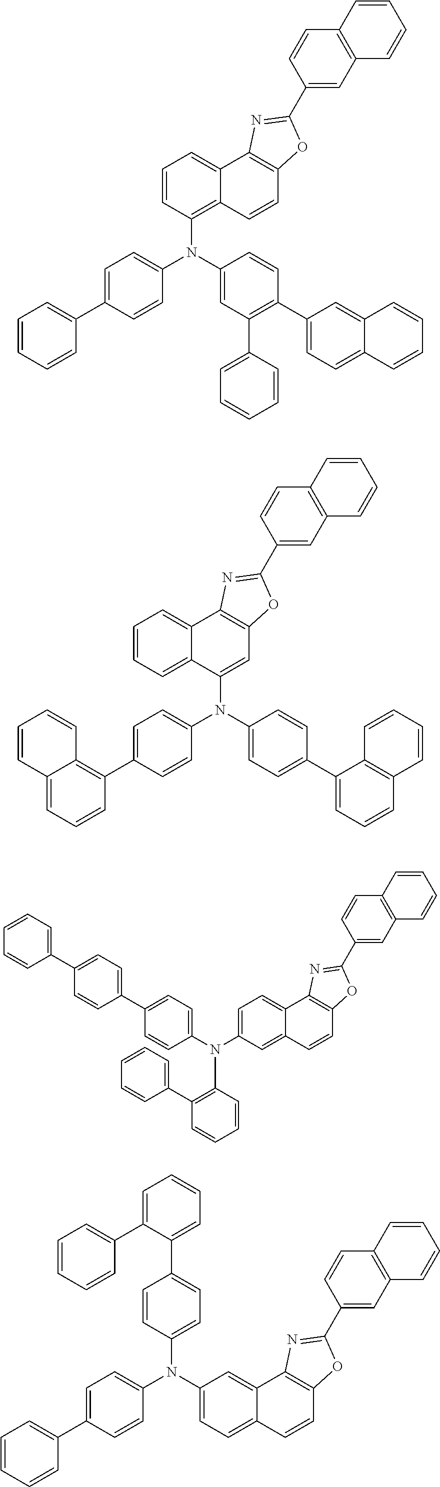 [SMILES: c1ccc(-c2ccc(N(c3ccc(-c4ccc5ccccc5c4)c(-c4ccccc4)c3)c3cccc4c3ccc3oc(-c5ccc6ccccc6c5)nc34)cc2)cc1 ; c1ccc2cc(-c3nc4c(cc(N(c5ccc(-c6cccc7ccccc67)cc5)c5ccc(-c6cccc7ccccc67)cc5)c5ccccc54)o3)ccc2c1 ; c1ccc(-c2ccc(-c3ccc(N(c4ccc5c(ccc6oc(-c7ccc8ccccc8c7)nc65)c4)c4ccccc4-c4ccccc4)cc3)cc2)cc1 ; c1ccc(-c2ccc(N(c3ccc(-c4ccccc4-c4ccccc4)cc3)c3ccc4ccc5oc(-c6ccc7ccccc7c6)nc5c4c3)cc2)cc1]

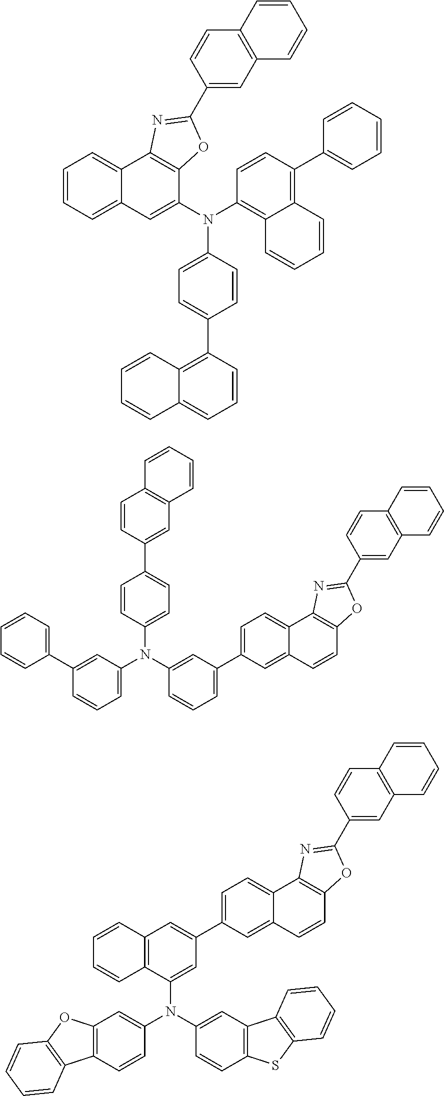

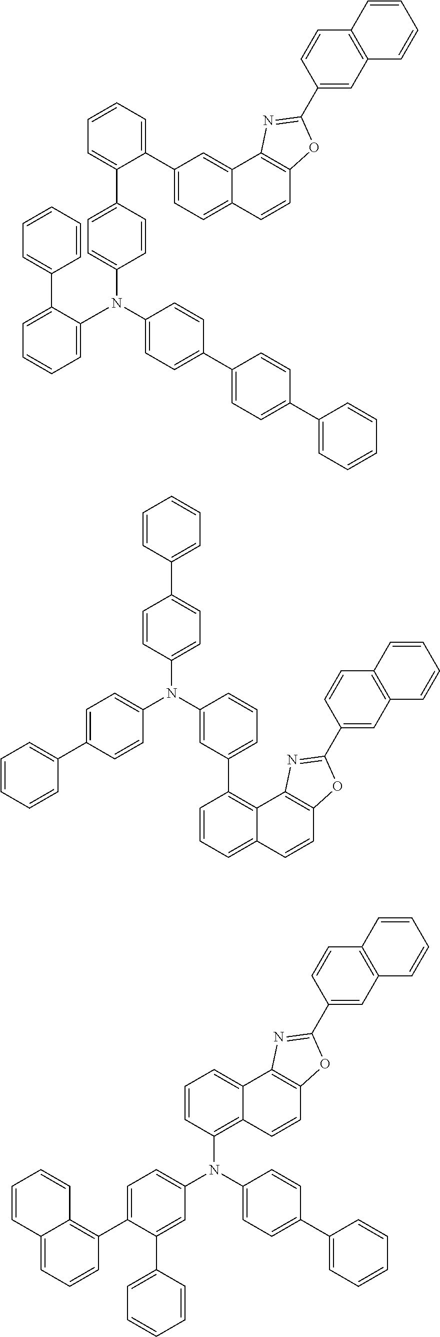 [SMILES: c1ccc(-c2ccc(-c3ccc(N(c4ccc(-c5ccccc5-c5ccc6ccc7oc(-c8ccc9ccccc9c8)nc7c6c5)cc4)c4ccccc4-c4ccccc4)cc3)cc2)cc1 ; c1ccc(-c2ccc(N(c3ccc(-c4ccccc4)cc3)c3cccc(-c4cccc5ccc6oc(-c7ccc8ccccc8c7)nc6c45)c3)cc2)cc1 ; c1ccc(-c2ccc(N(c3ccc(-c4cccc5ccccc45)c(-c4ccccc4)c3)c3cccc4c3ccc3oc(-c5ccc6ccccc6c5)nc34)cc2)cc1]

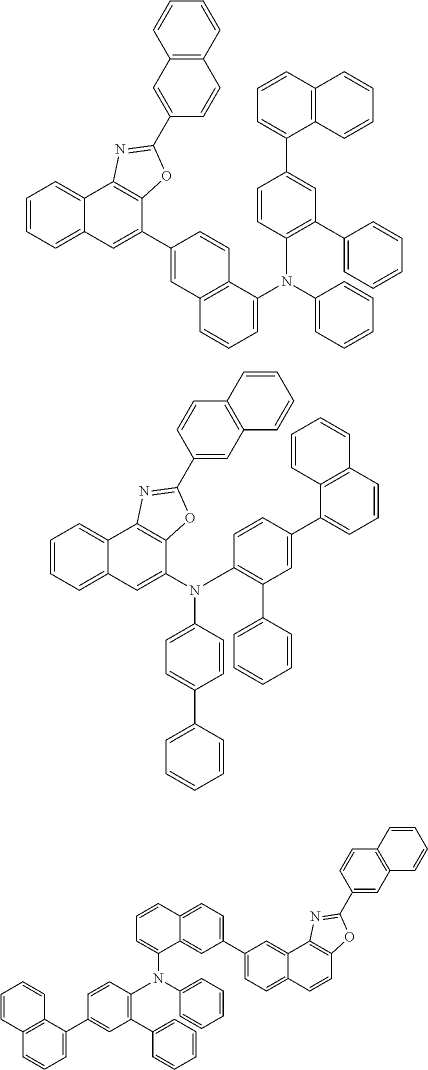

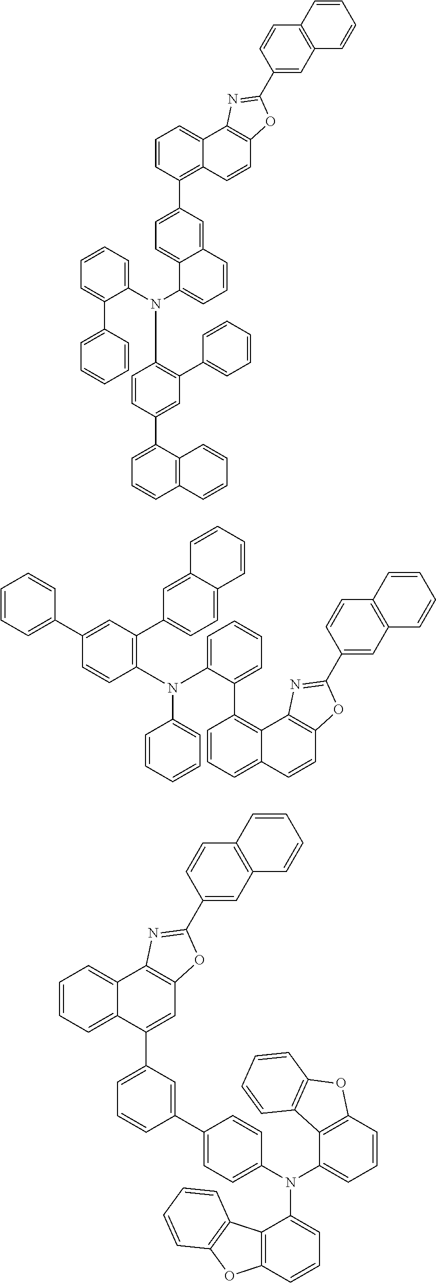

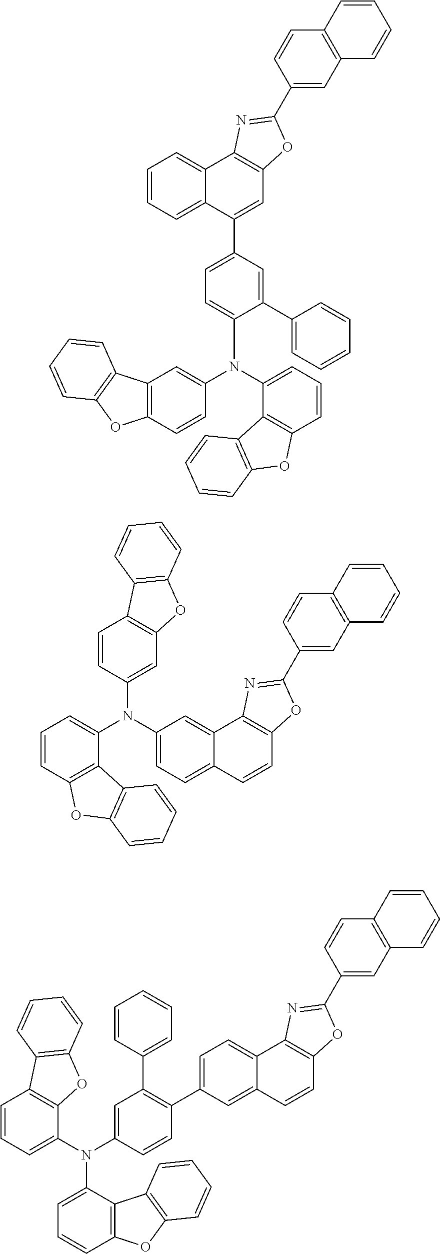 [SMILES: c1ccc(-c2cc(-c3cc4oc(-c5ccc6ccccc6c5)nc4c4ccccc34)ccc2N(c2ccc3oc4ccccc4c3c2)c2cccc3oc4ccccc4c23)cc1 ; c1ccc2cc(-c3nc4c(ccc5ccc(N(c6ccc7c(c6)oc6ccccc67)c6cccc7oc8ccccc8c67)cc54)o3)ccc2c1 ; c1ccc(-c2cc(N(c3cccc4c3oc3ccccc34)c3cccc4oc5ccccc5c34)ccc2-c2ccc3c(ccc4oc(-c5ccc6ccccc6c5)nc43)c2)cc1]

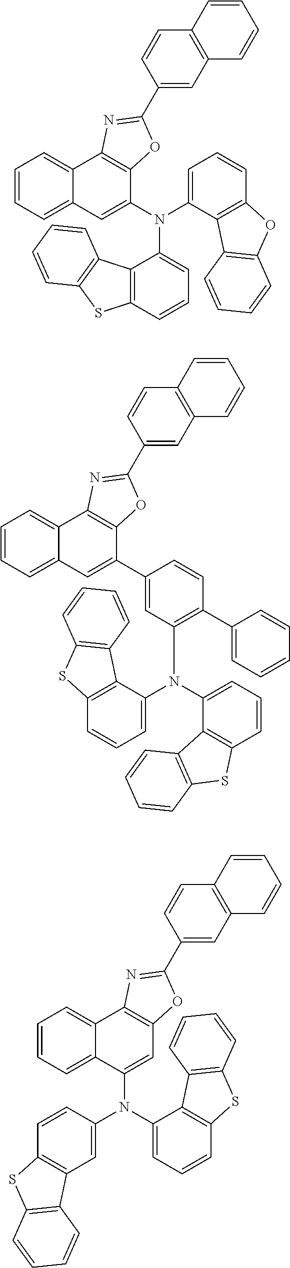 [SMILES: c1ccc2cc(-c3nc4c(o3)c(N(c3cccc5oc6ccccc6c35)c3cccc5sc6ccccc6c35)cc3ccccc34)ccc2c1 ; c1ccc(-c2ccc(-c3cc4ccccc4c4nc(-c5ccc6ccccc6c5)oc34)cc2N(c2cccc3sc4ccccc4c23)c2cccc3sc4ccccc4c23)cc1 ; c1ccc2cc(-c3nc4c(cc(N(c5ccc6sc7ccccc7c6c5)c5cccc6sc7ccccc7c56)c5ccccc54)o3)ccc2c1]

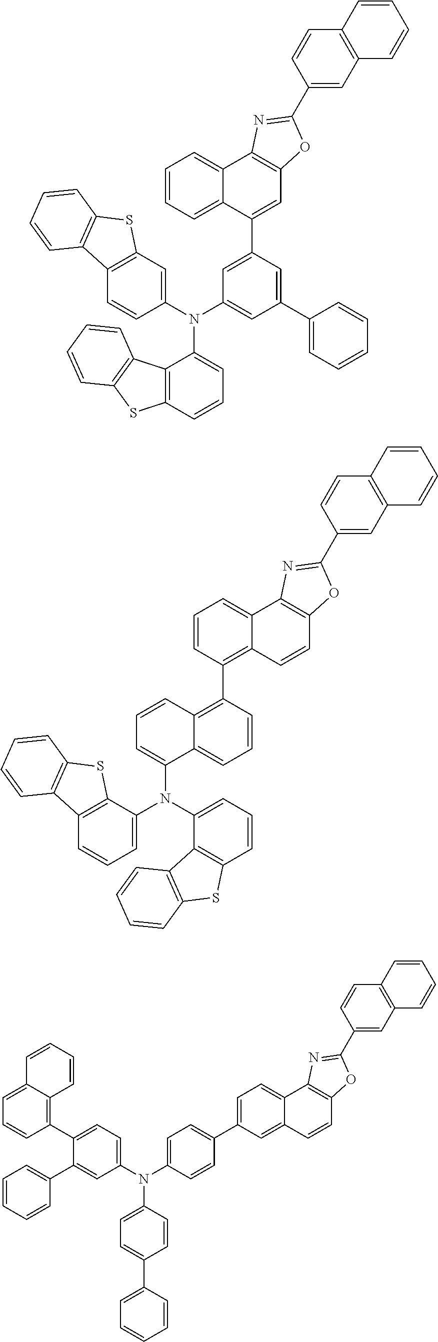 [SMILES: c1ccc(-c2cc(-c3cc4oc(-c5ccc6ccccc6c5)nc4c4ccccc34)cc(N(c3ccc4c(c3)sc3ccccc34)c3cccc4sc5ccccc5c34)c2)cc1 ; c1ccc2cc(-c3nc4c(ccc5c(-c6cccc7c(N(c8cccc9c8sc8ccccc89)c8cccc9sc%10ccccc%10c89)cccc67)cccc54)o3)ccc2c1 ; c1ccc(-c2ccc(N(c3ccc(-c4ccc5c(ccc6oc(-c7ccc8ccccc8c7)nc65)c4)cc3)c3ccc(-c4cccc5ccccc45)c(-c4ccccc4)c3)cc2)cc1]

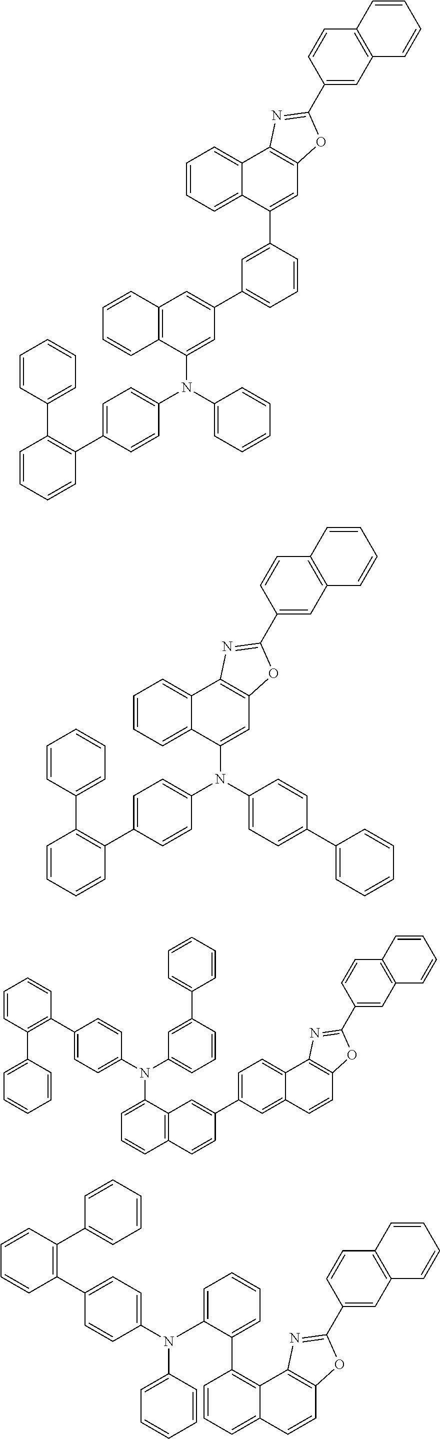

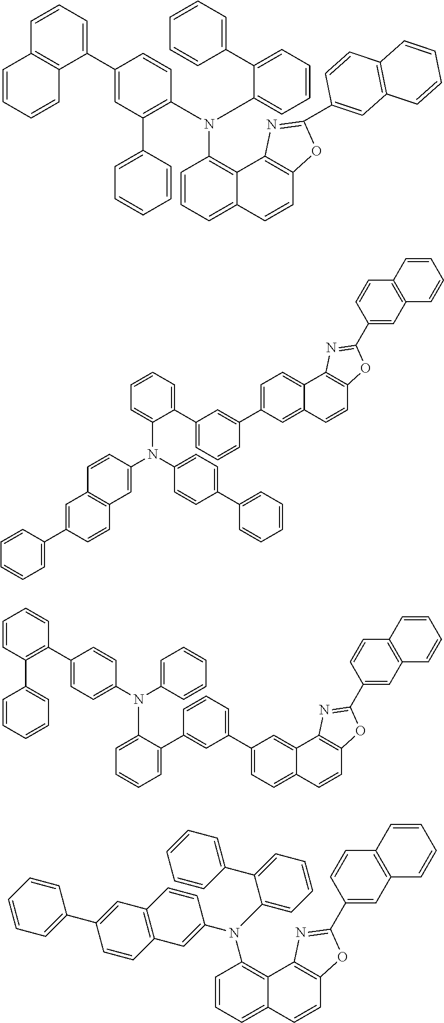

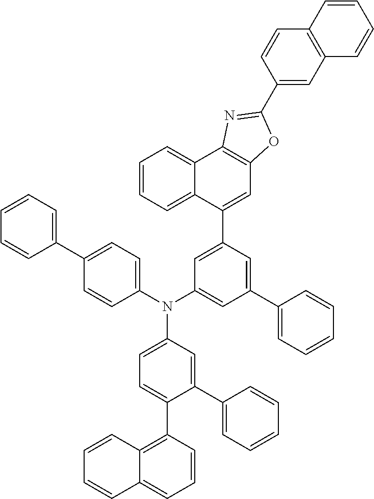 [SMILES: c1ccc(-c2ccc(N(c3cc(-c4ccccc4)cc(-c4cc5oc(-c6ccc7ccccc7c6)nc5c5ccccc45)c3)c3ccc(-c4cccc5ccccc45)c(-c4ccccc4)c3)cc2)cc1]

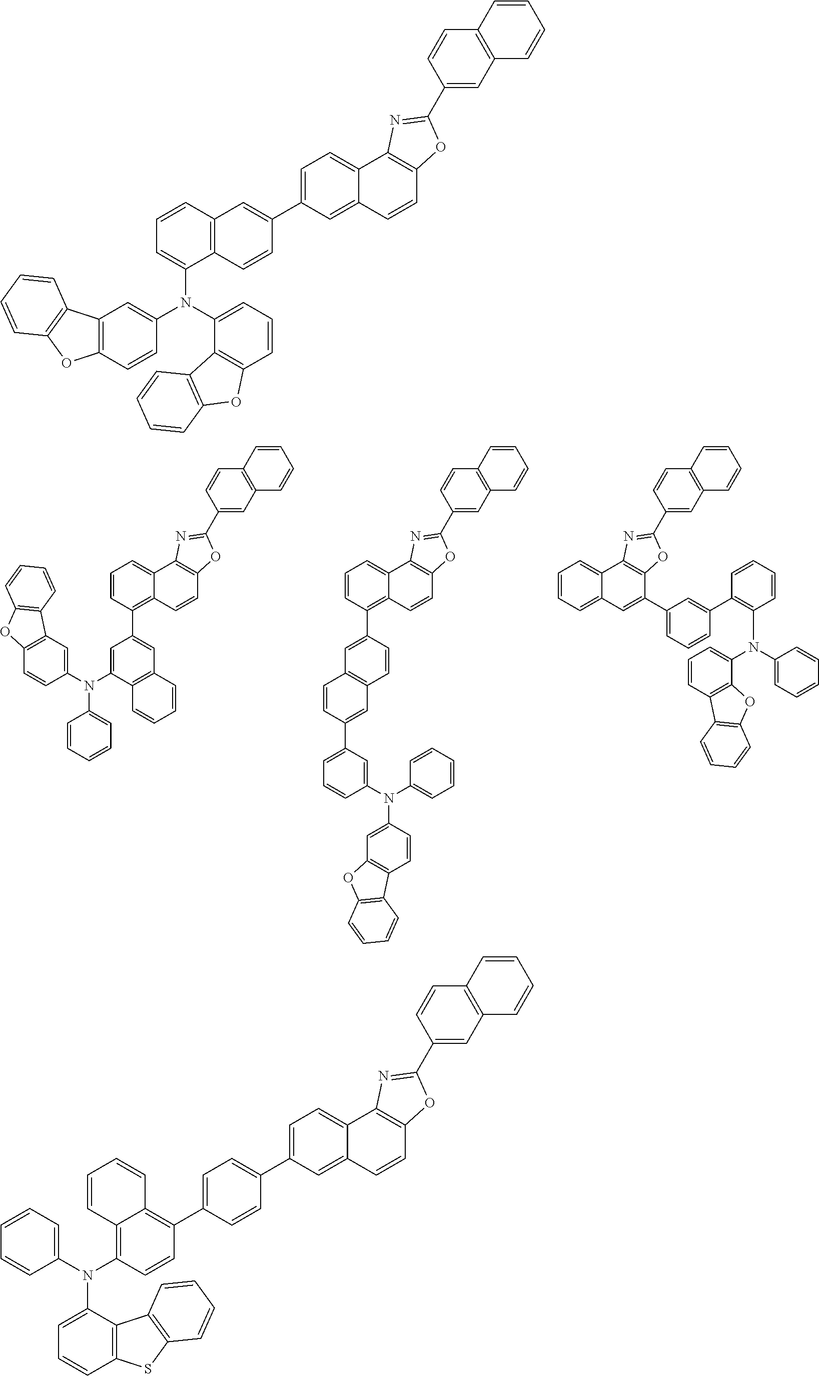 [SMILES: c1ccc2cc(-c3nc4c(ccc5cc(-c6ccc7c(N(c8ccc9oc%10ccccc%10c9c8)c8cccc9oc%10ccccc%10c89)cccc7c6)ccc54)o3)ccc2c1 ; c1ccc(N(c2ccc3oc4ccccc4c3c2)c2cc(-c3cccc4c3ccc3oc(-c5ccc6ccccc6c5)nc34)cc3ccccc23)cc1 ; c1ccc(N(c2cccc(-c3ccc4cc(-c5cccc6c5ccc5oc(-c7ccc8ccccc8c7)nc56)ccc4c3)c2)c2ccc3c(c2)oc2ccccc23)cc1 ; c1ccc(N(c2ccccc2-c2cccc(-c3cc4ccccc4c4nc(-c5ccc6ccccc6c5)oc34)c2)c2cccc3c2oc2ccccc23)cc1 ; c1ccc(N(c2ccc(-c3ccc(-c4ccc5c(ccc6oc(-c7ccc8ccccc8c7)nc65)c4)cc3)c3ccccc23)c2cccc3sc4ccccc4c23)cc1]

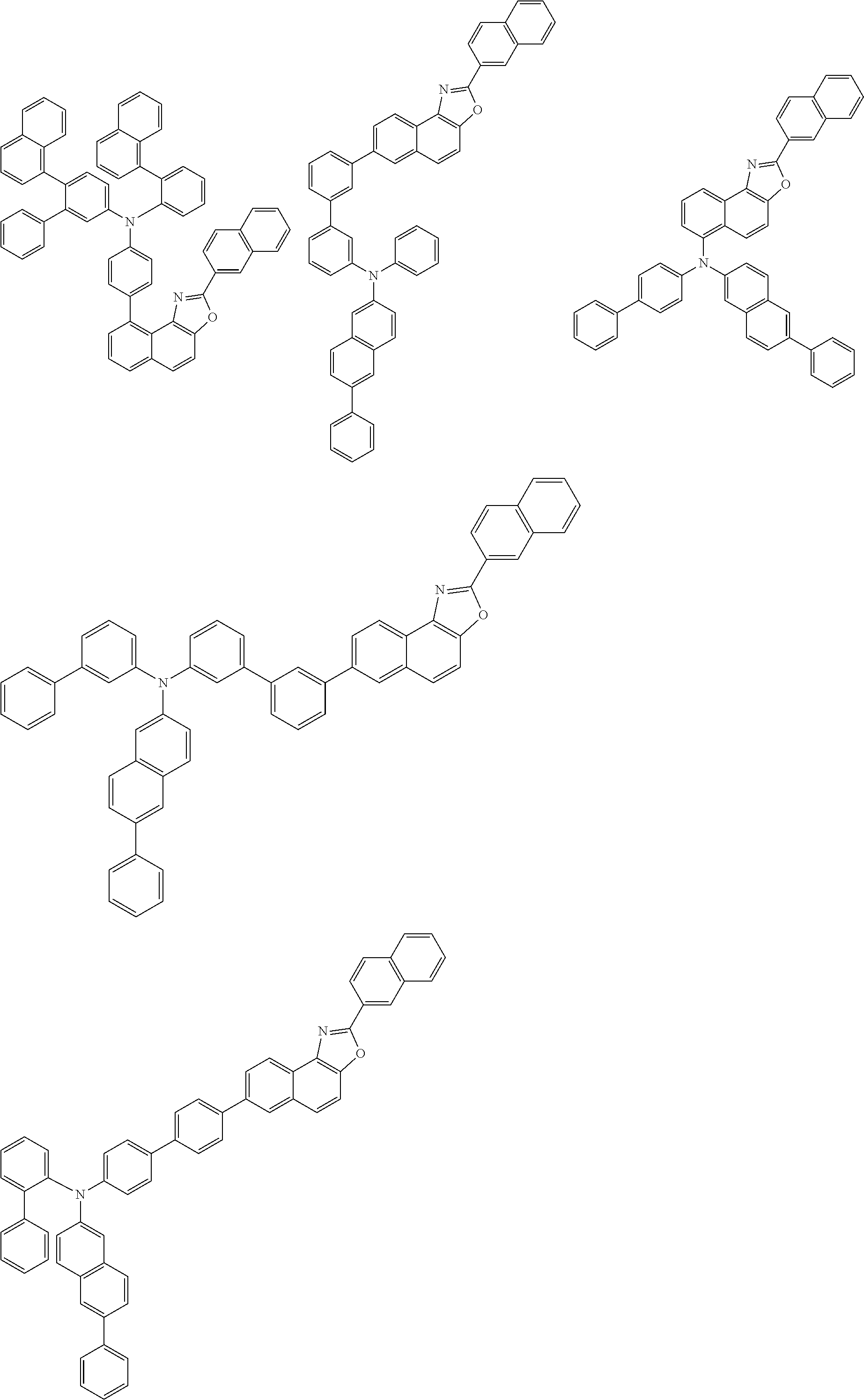

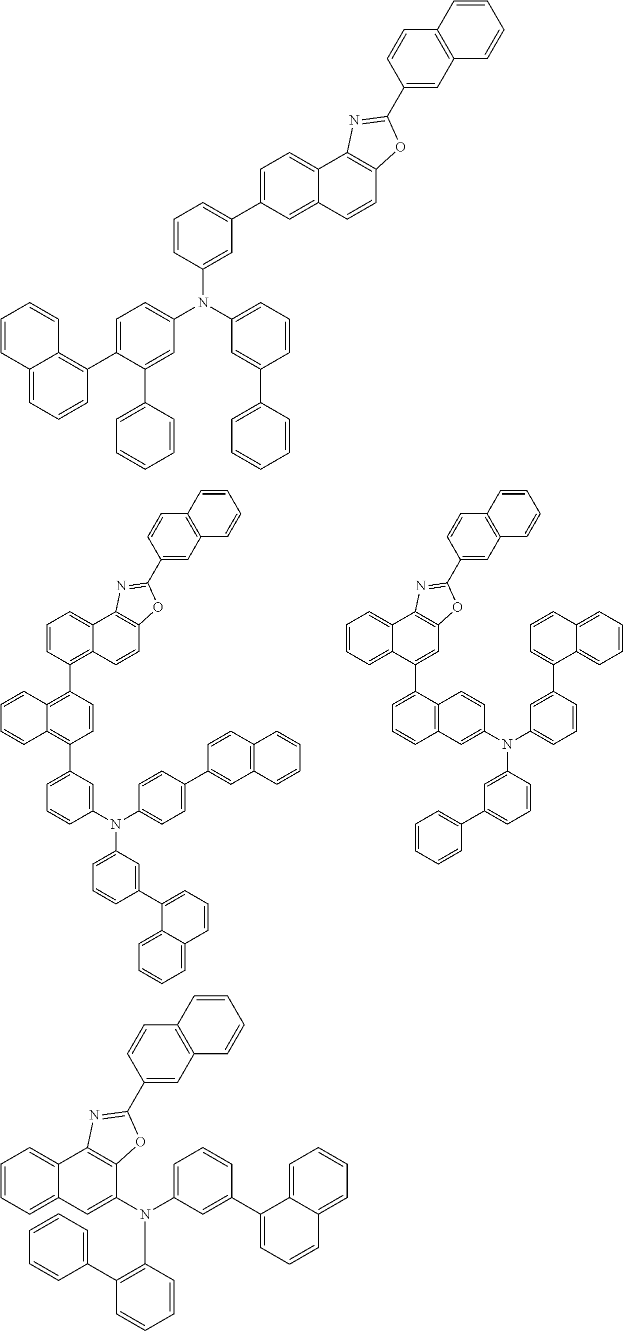

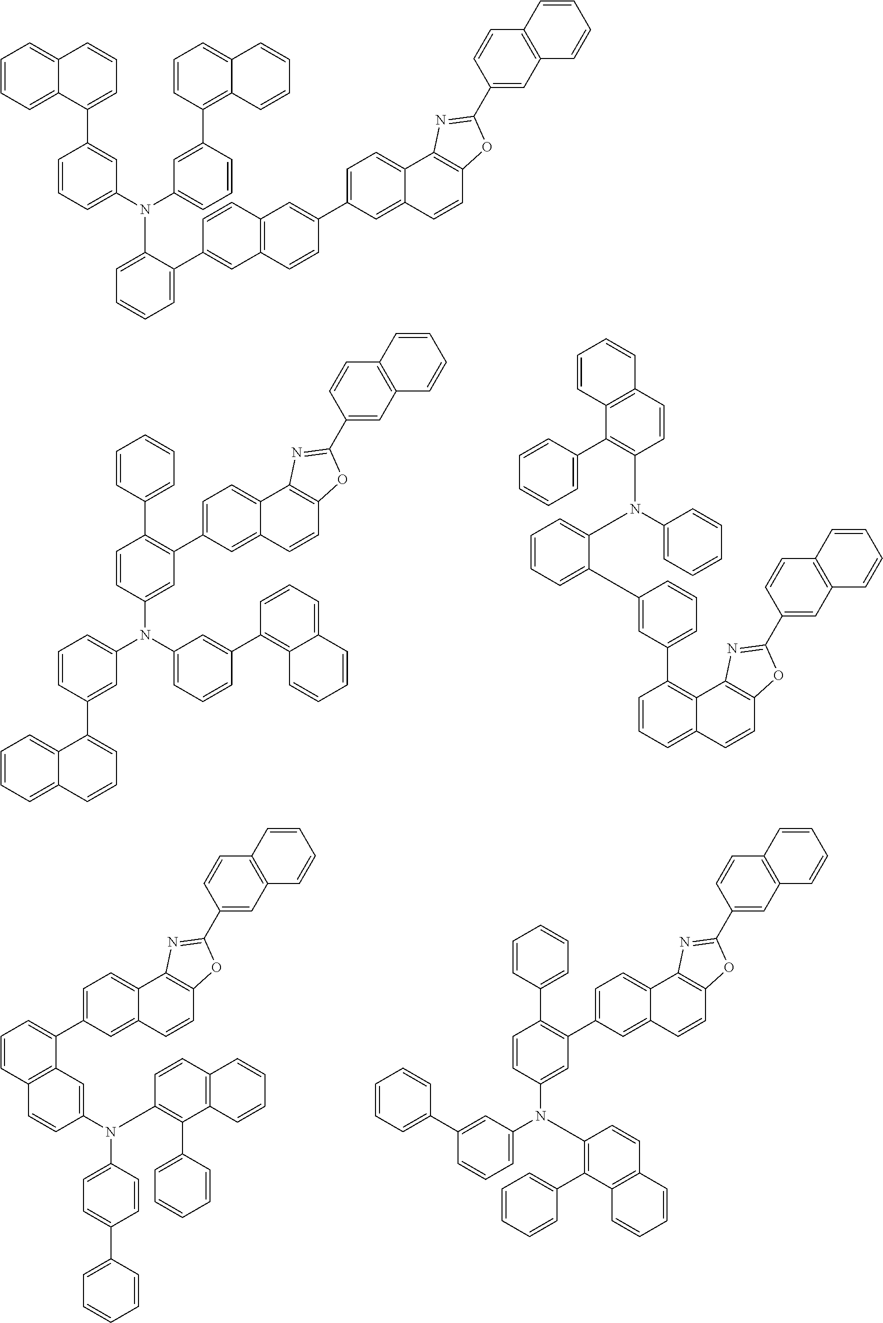

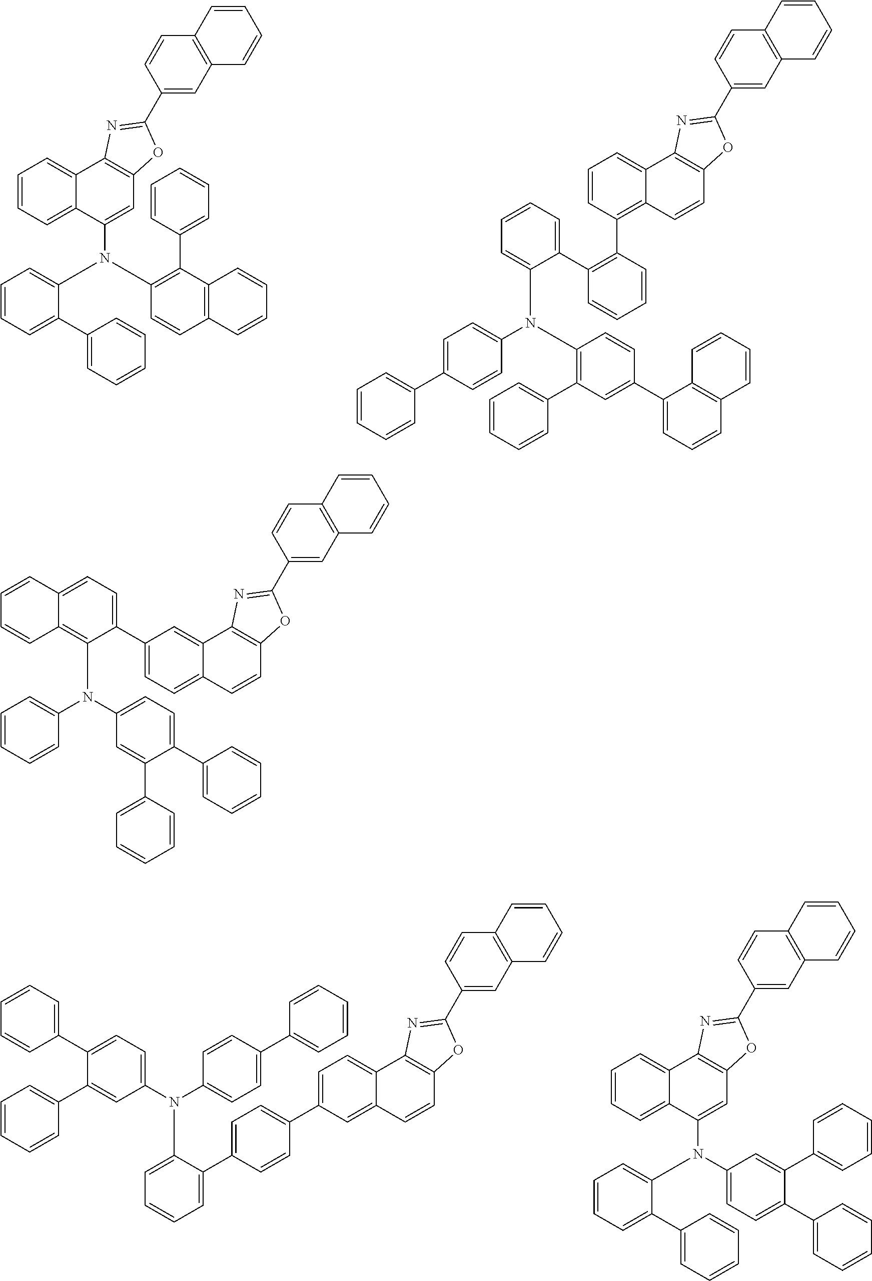

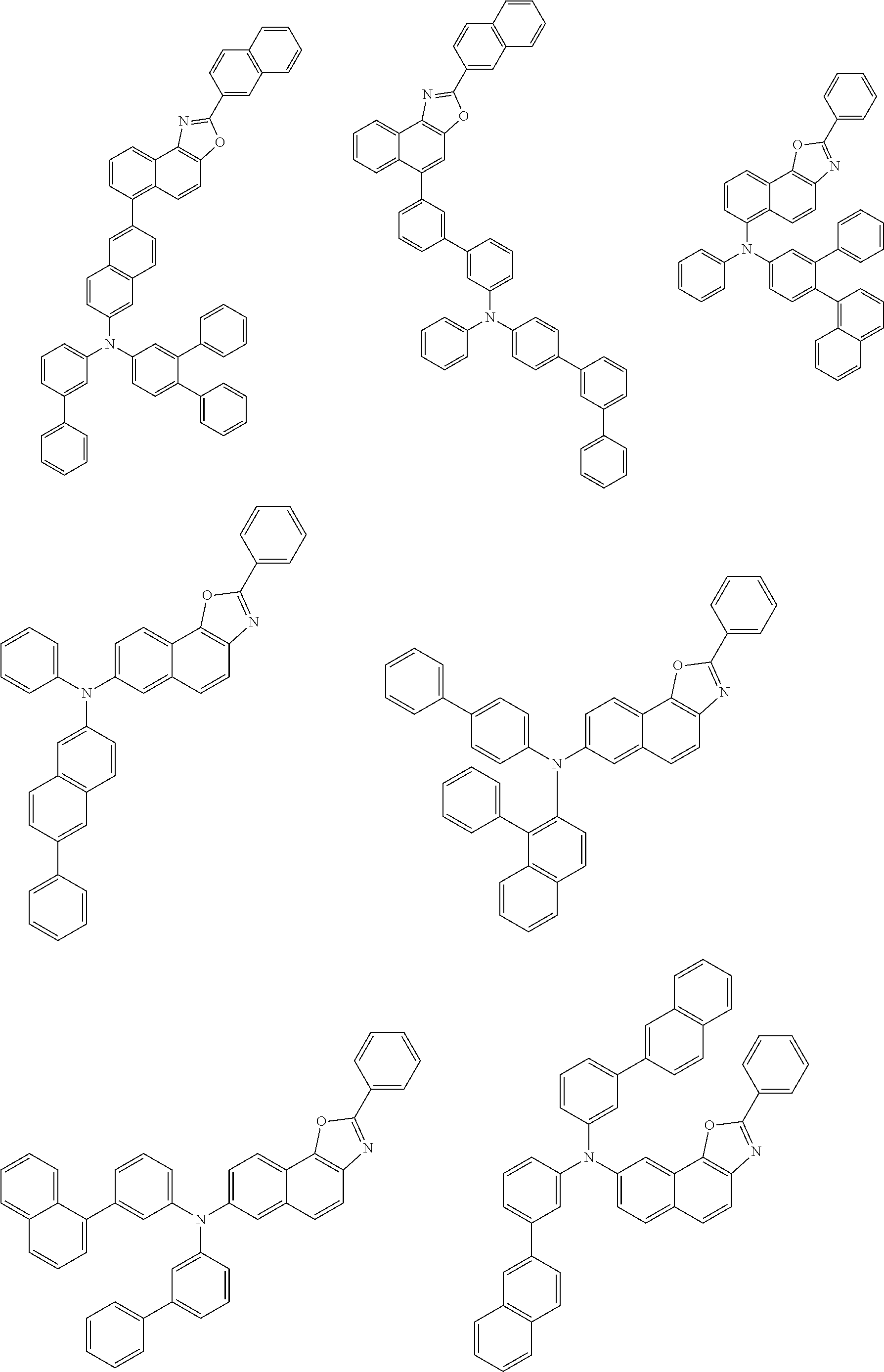

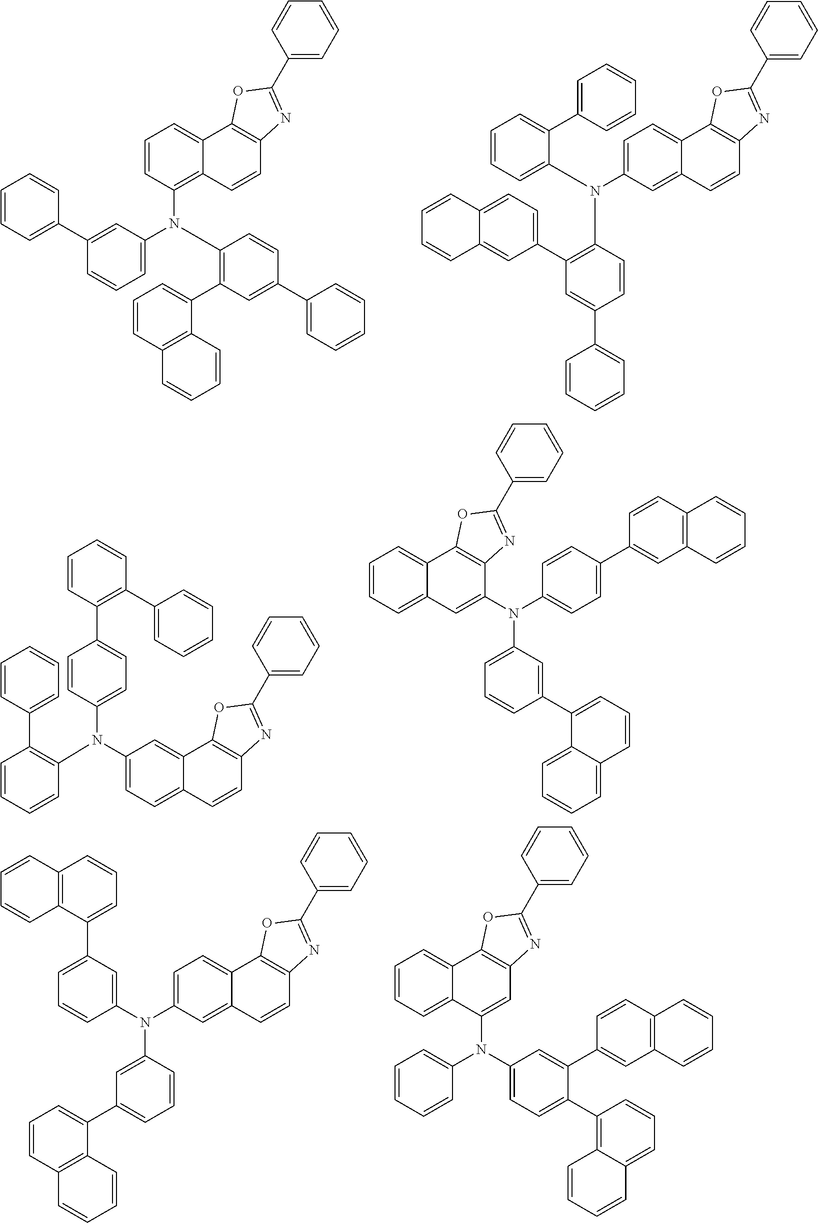

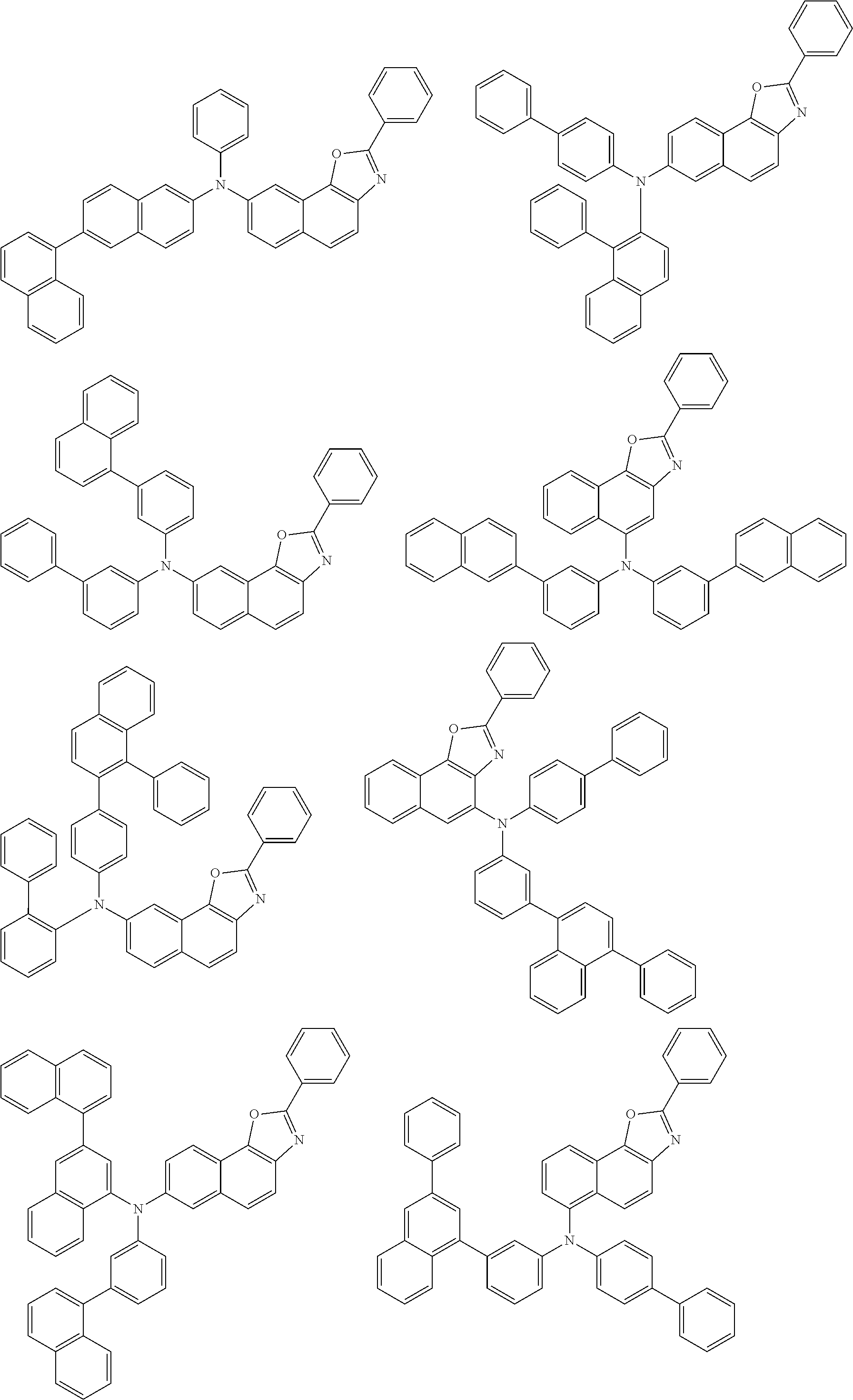

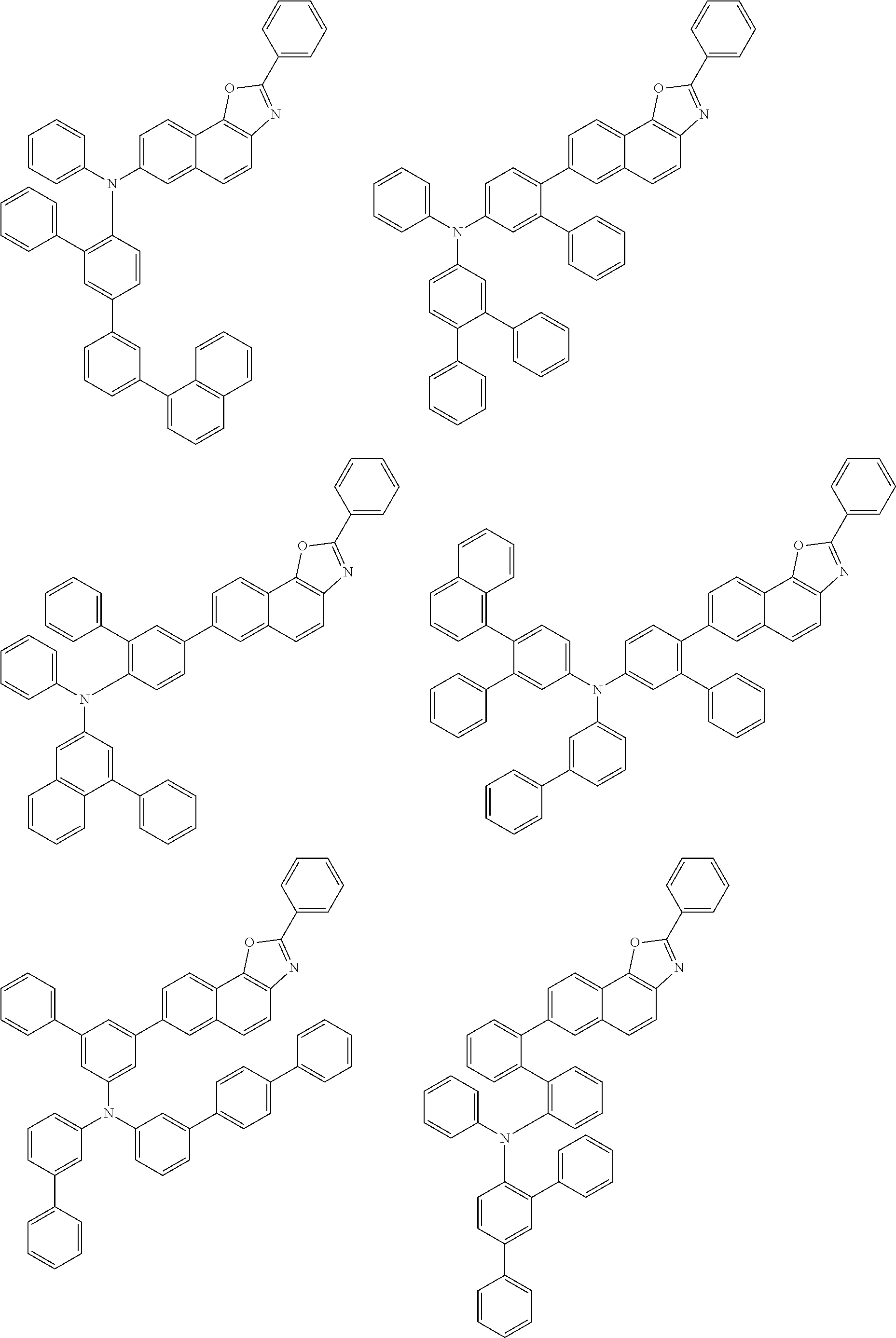

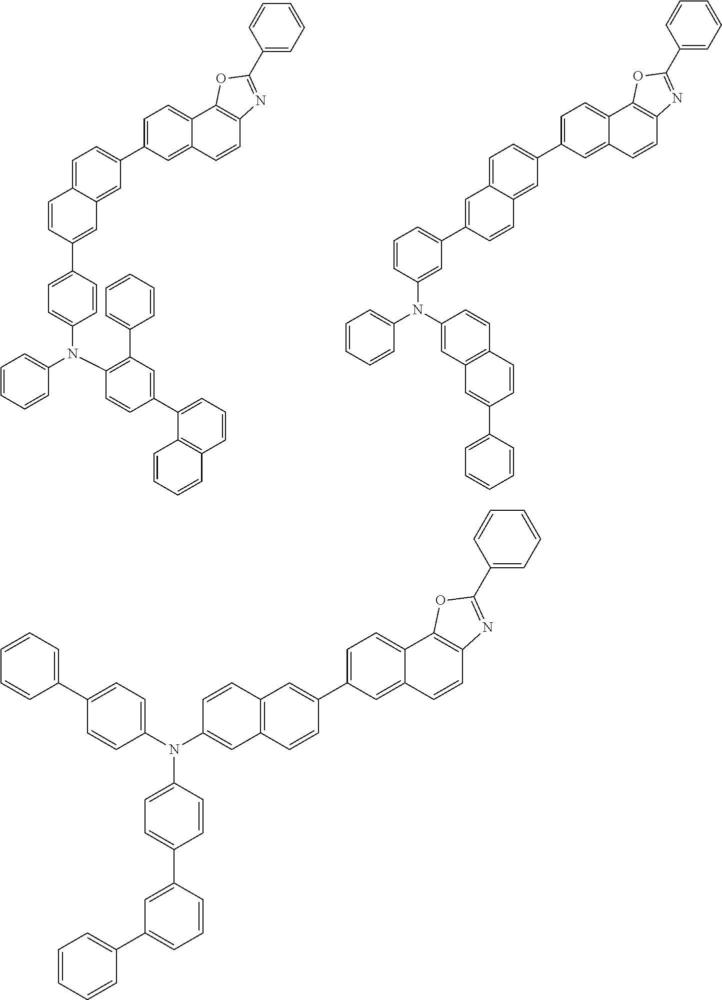

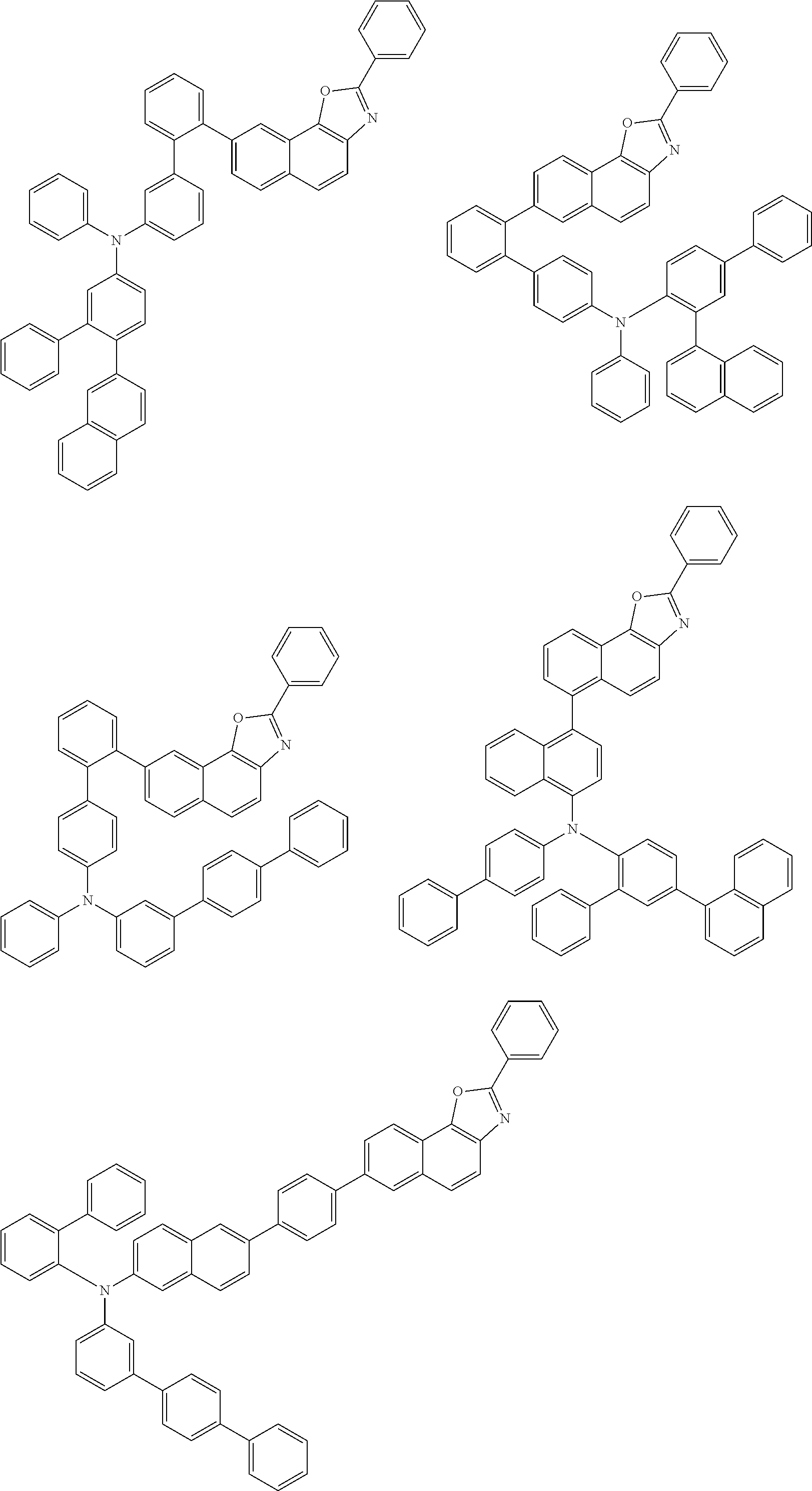

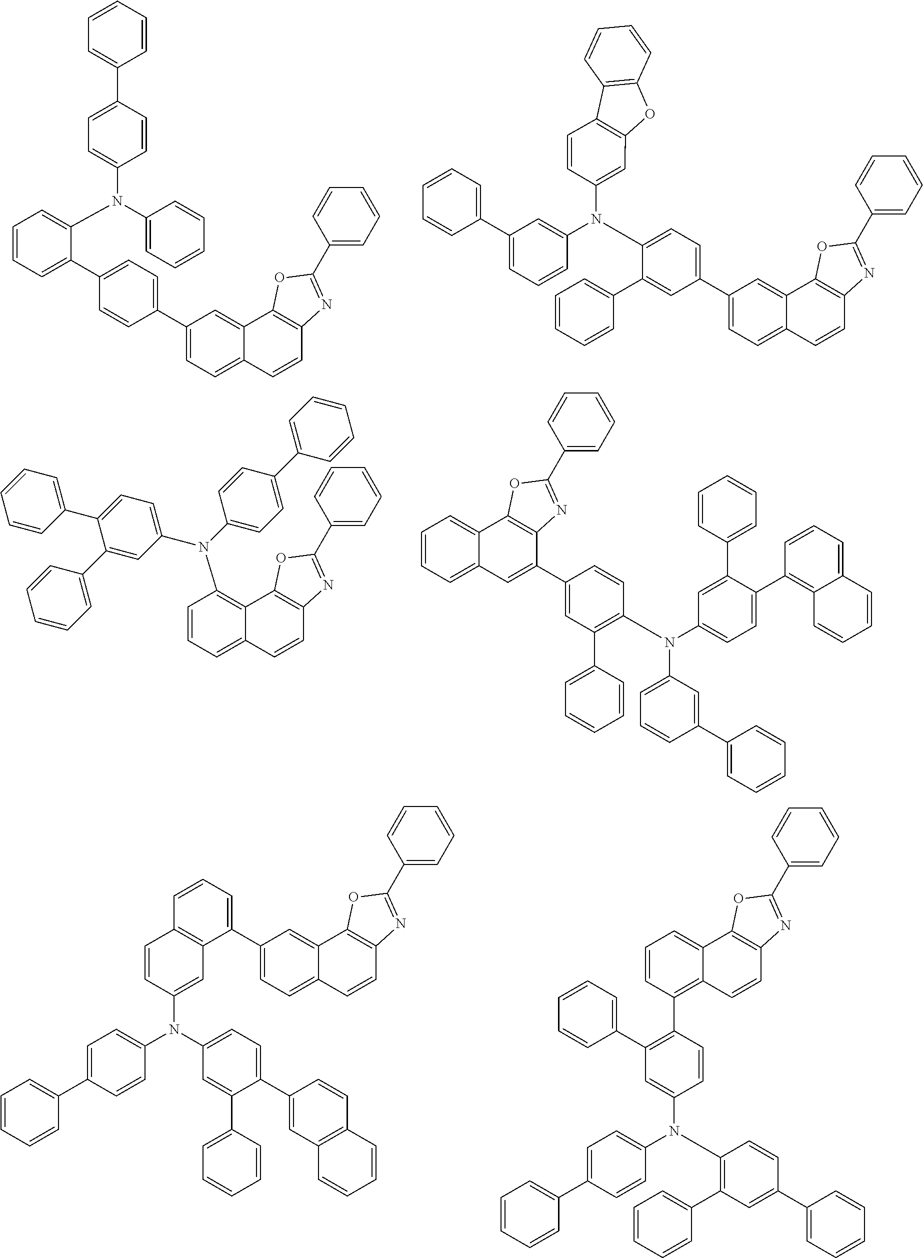

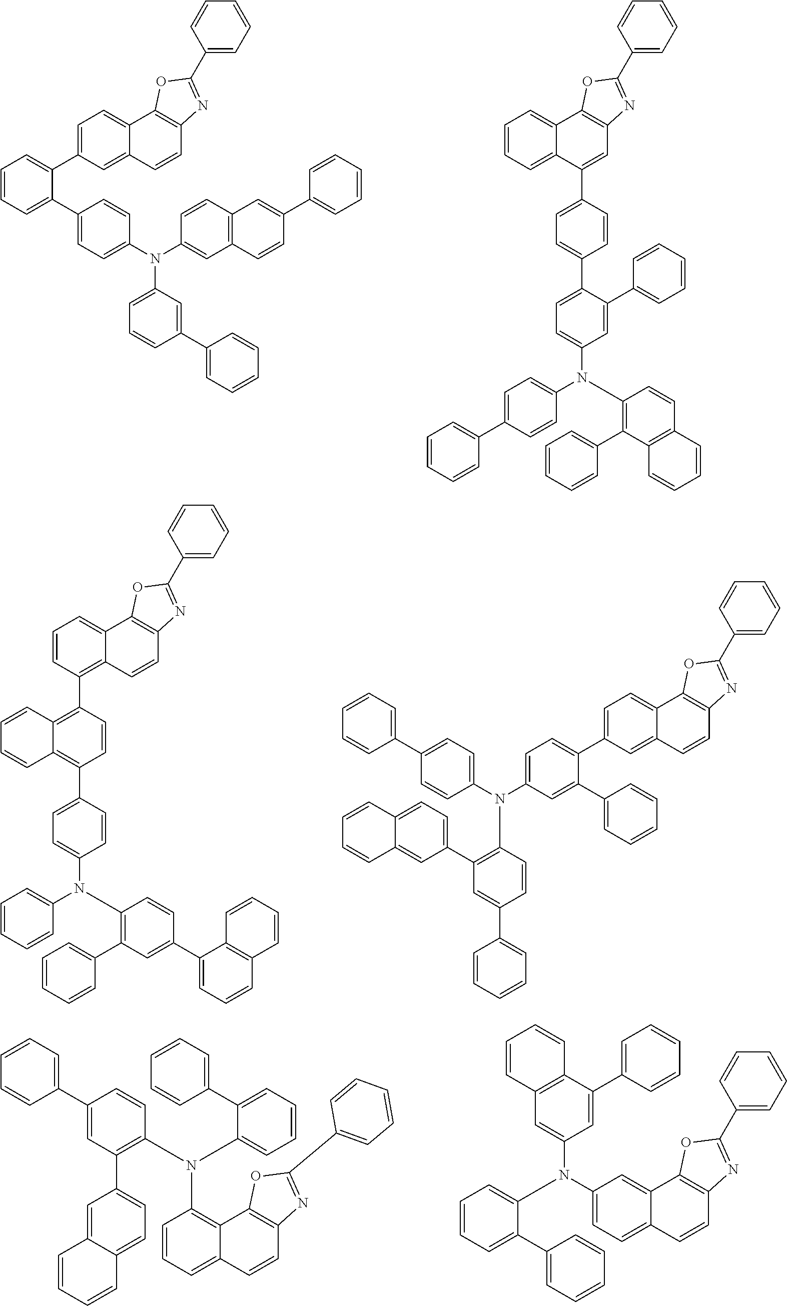

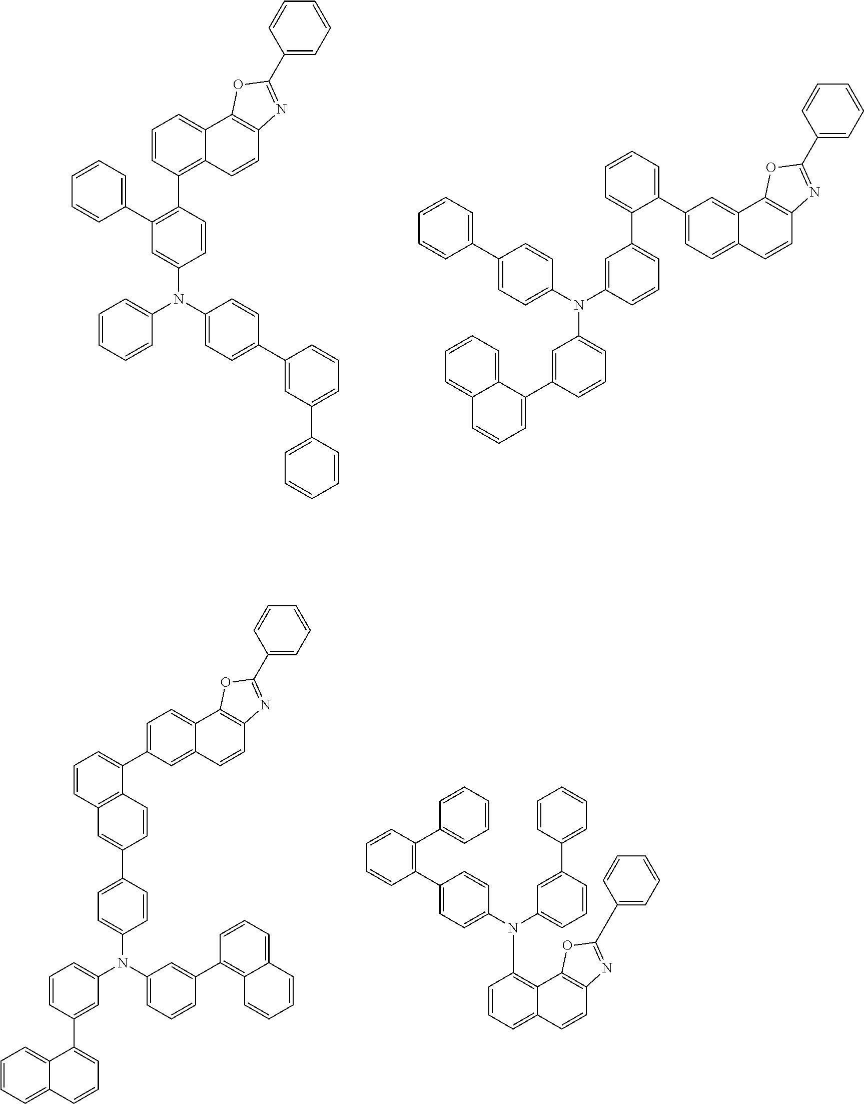 [SMILES: c1ccc(-c2cccc(-c3ccc(N(c4ccccc4)c4ccc(-c5cccc6c5ccc5nc(-c7ccccc7)oc56)c(-c5ccccc5)c4)cc3)c2)cc1 ; c1ccc(-c2ccc(N(c3cccc(-c4ccccc4-c4ccc5ccc6nc(-c7ccccc7)oc6c5c4)c3)c3cccc(-c4cccc5ccccc45)c3)cc2)cc1 ; c1ccc(-c2nc3ccc4cc(-c5cccc6cc(-c7ccc(N(c8cccc(-c9cccc%10ccccc9%10)c8)c8cccc(-c9cccc%10ccccc9%10)c8)cc7)ccc56)ccc4c3o2)cc1 ; c1ccc(-c2cccc(N(c3ccc(-c4ccccc4-c4ccccc4)cc3)c3cccc4ccc5nc(-c6ccccc6)oc5c34)c2)cc1]

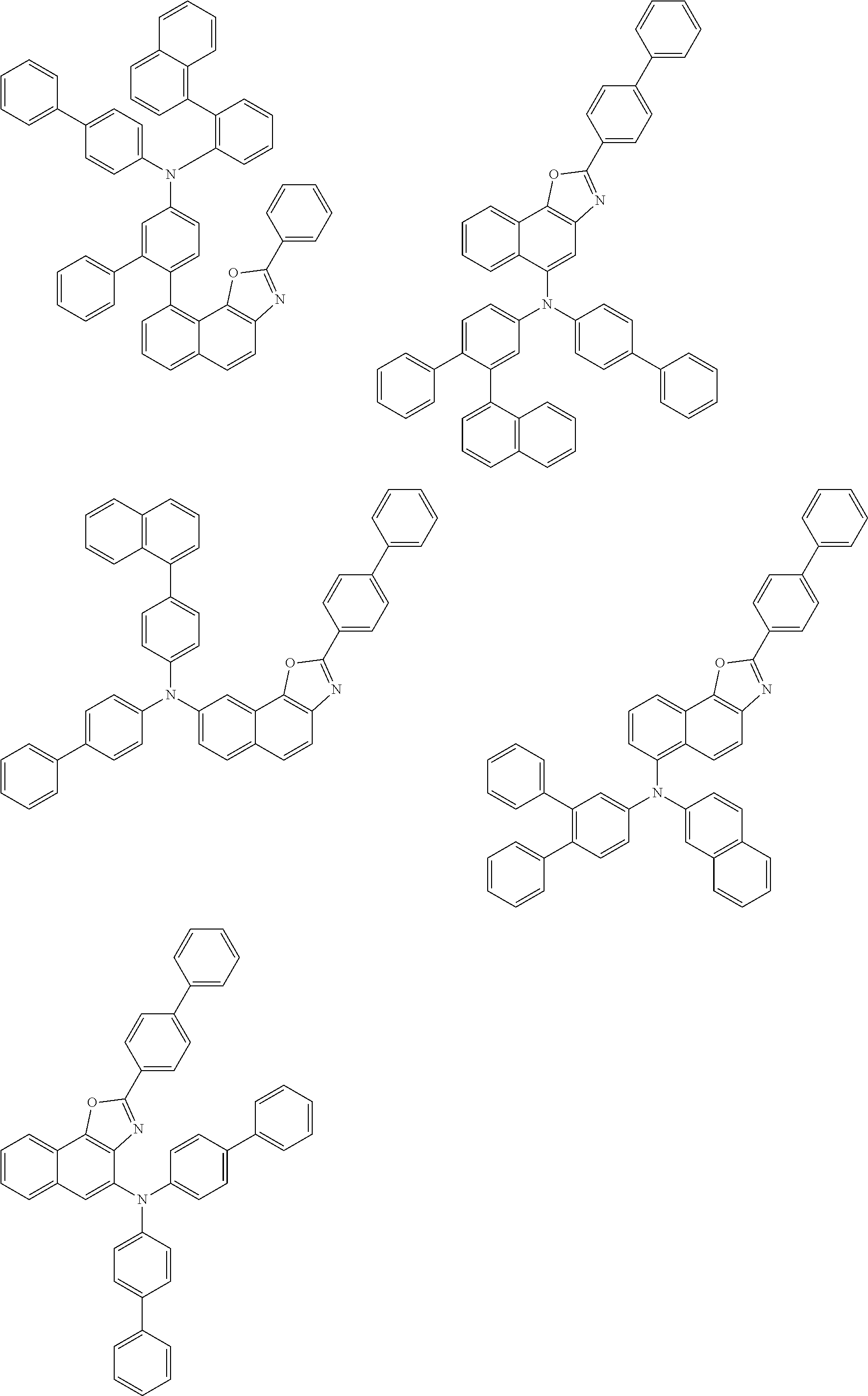 [SMILES: c1ccc(-c2ccc(N(c3ccc(-c4cccc5ccc6nc(-c7ccccc7)oc6c45)c(-c4ccccc4)c3)c3ccccc3-c3cccc4ccccc34)cc2)cc1 ; c1ccc(-c2ccc(-c3nc4cc(N(c5ccc(-c6ccccc6)cc5)c5ccc(-c6ccccc6)c(-c6cccc7ccccc67)c5)c5ccccc5c4o3)cc2)cc1 ; c1ccc(-c2ccc(-c3nc4ccc5ccc(N(c6ccc(-c7ccccc7)cc6)c6ccc(-c7cccc8ccccc78)cc6)cc5c4o3)cc2)cc1 ; c1ccc(-c2ccc(-c3nc4ccc5c(N(c6ccc(-c7ccccc7)c(-c7ccccc7)c6)c6ccc7ccccc7c6)cccc5c4o3)cc2)cc1 ; c1ccc(-c2ccc(-c3nc4c(N(c5ccc(-c6ccccc6)cc5)c5ccc(-c6ccccc6)cc5)cc5ccccc5c4o3)cc2)cc1]

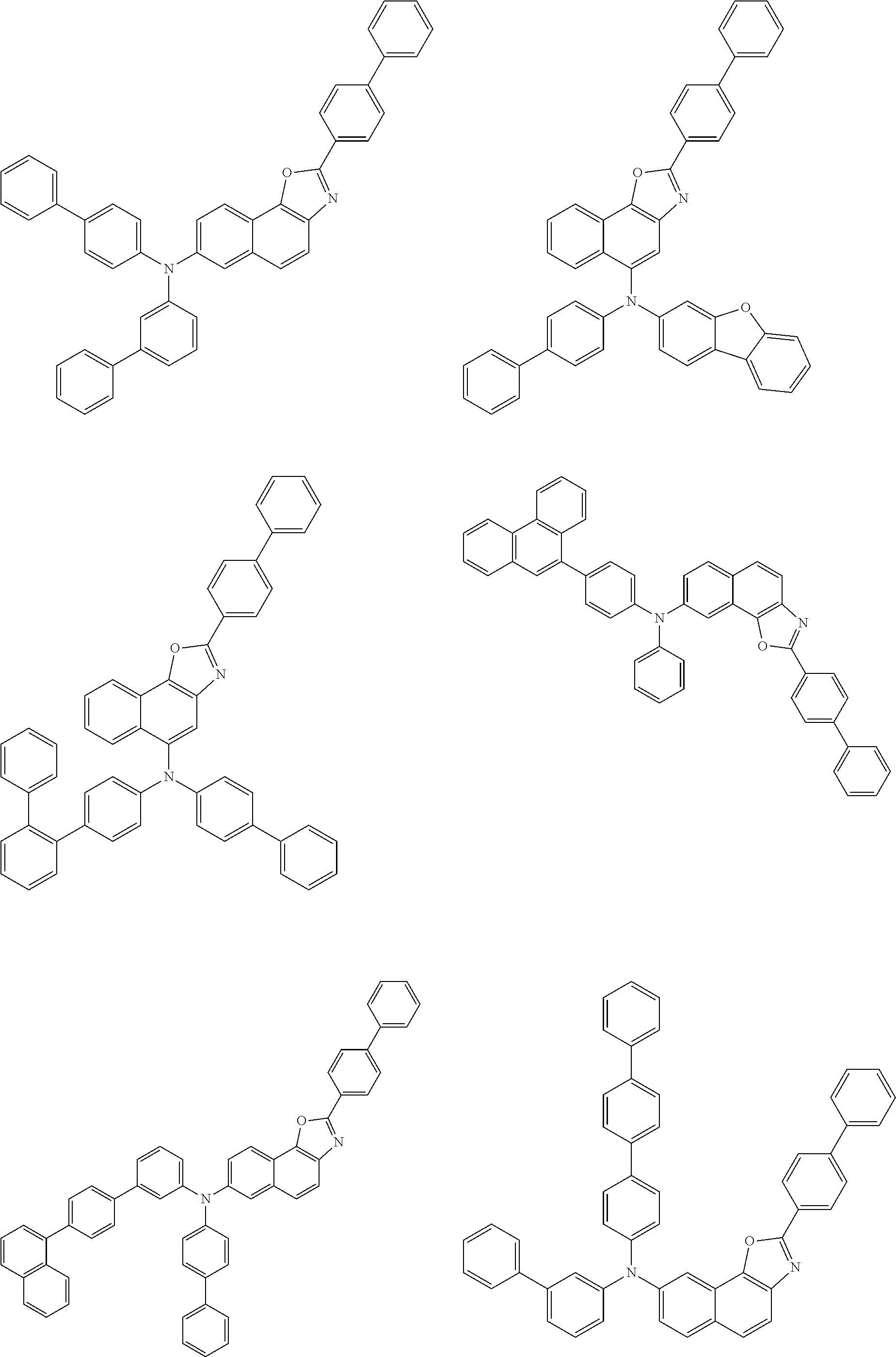

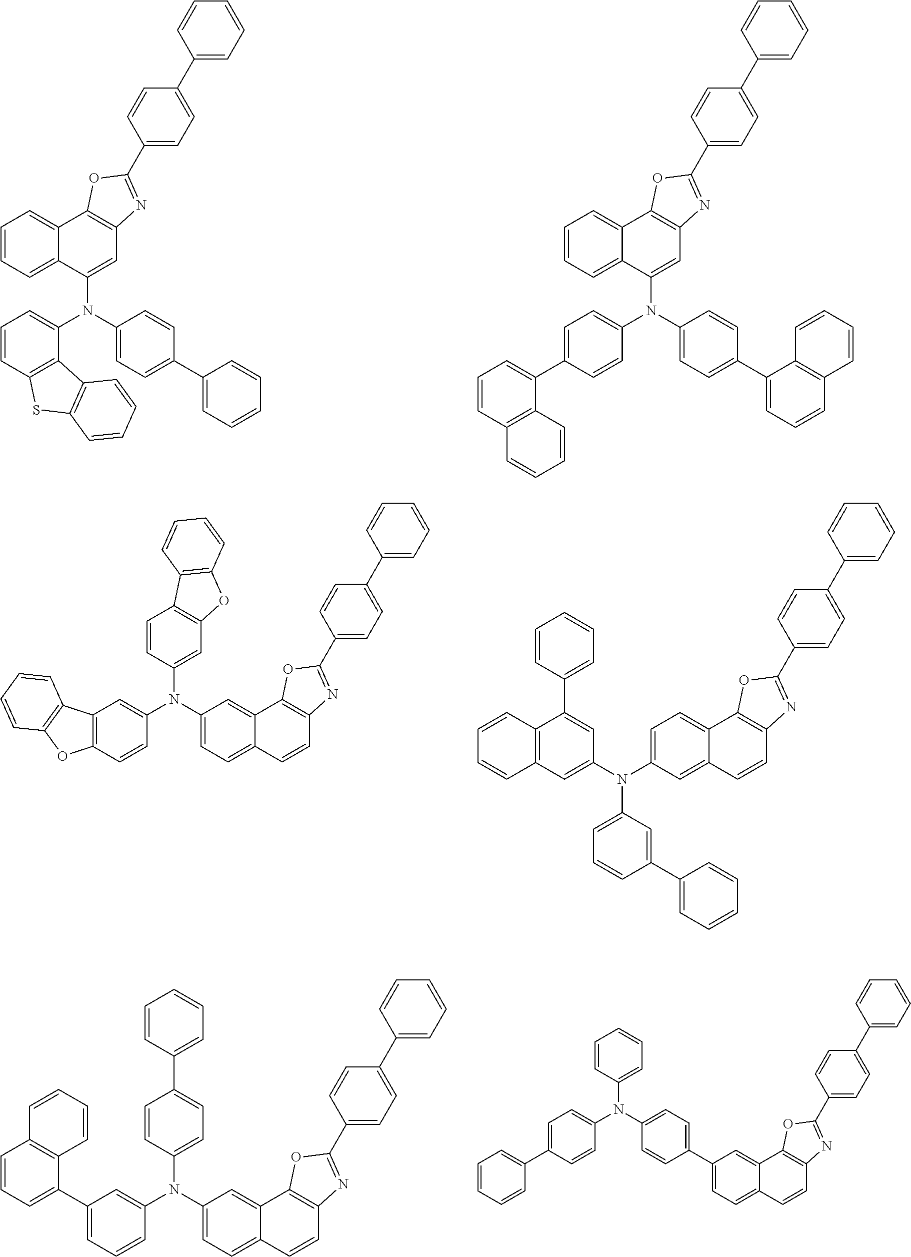

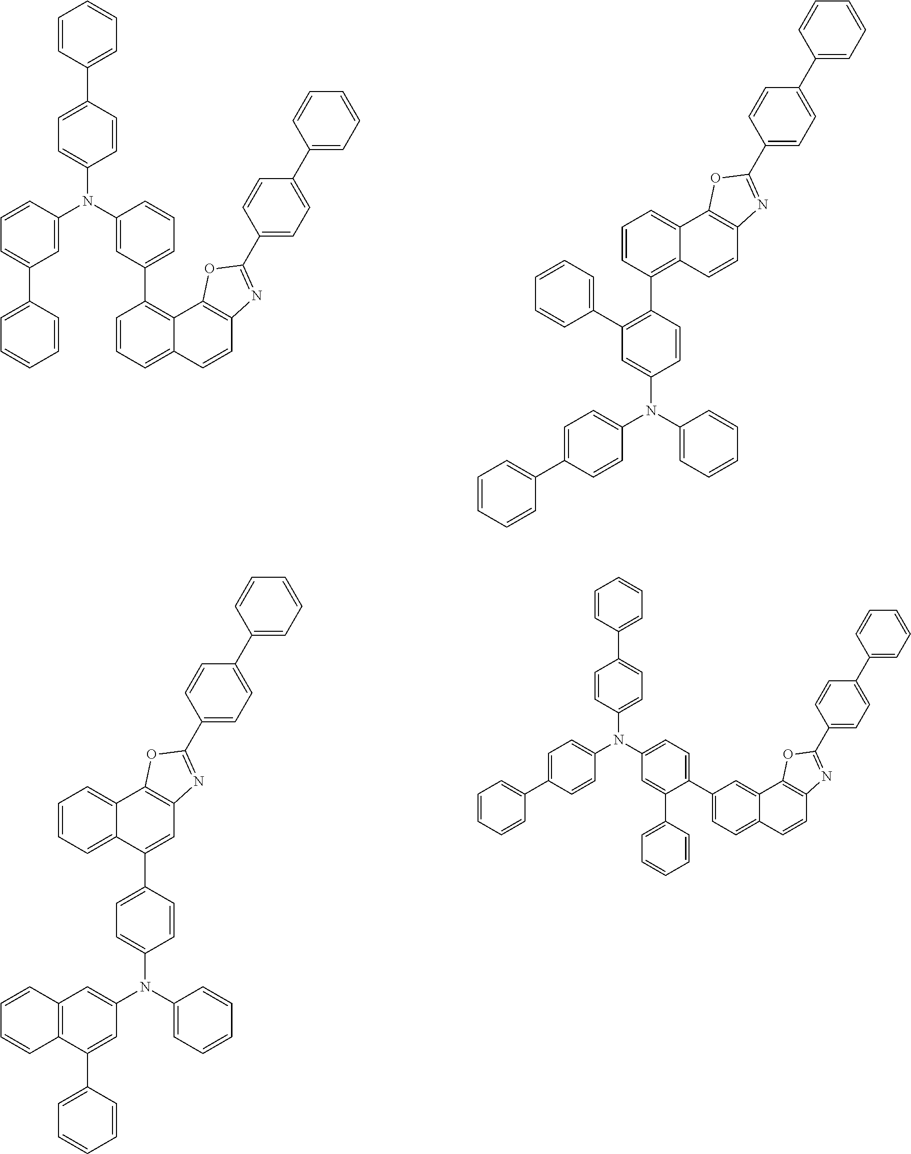 [SMILES: c1ccc(-c2ccc(-c3nc4ccc5cccc(-c6cccc(N(c7ccc(-c8ccccc8)cc7)c7cccc(-c8ccccc8)c7)c6)c5c4o3)cc2)cc1 ; c1ccc(-c2ccc(-c3nc4ccc5c(-c6ccc(N(c7ccccc7)c7ccc(-c8ccccc8)cc7)cc6-c6ccccc6)cccc5c4o3)cc2)cc1 ; c1ccc(-c2ccc(-c3nc4cc(-c5ccc(N(c6ccccc6)c6cc(-c7ccccc7)c7ccccc7c6)cc5)c5ccccc5c4o3)cc2)cc1 ; c1ccc(-c2ccc(-c3nc4ccc5ccc(-c6ccc(N(c7ccc(-c8ccccc8)cc7)c7ccc(-c8ccccc8)cc7)cc6-c6ccccc6)cc5c4o3)cc2)cc1]

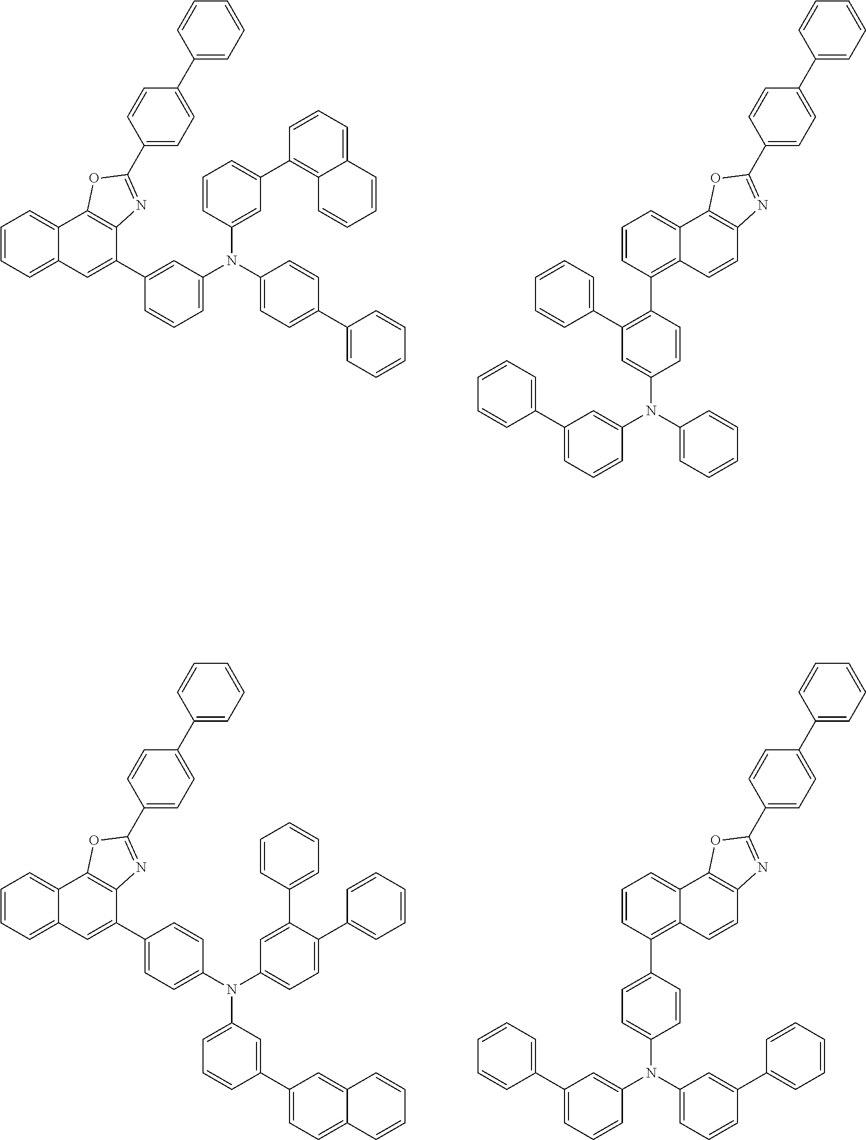 [SMILES: c1ccc(-c2ccc(-c3nc4c(-c5cccc(N(c6ccc(-c7ccccc7)cc6)c6cccc(-c7cccc8ccccc78)c6)c5)cc5ccccc5c4o3)cc2)cc1 ; c1ccc(-c2ccc(-c3nc4ccc5c(-c6ccc(N(c7ccccc7)c7cccc(-c8ccccc8)c7)cc6-c6ccccc6)cccc5c4o3)cc2)cc1 ; c1ccc(-c2ccc(-c3nc4c(-c5ccc(N(c6cccc(-c7ccc8ccccc8c7)c6)c6ccc(-c7ccccc7)c(-c7ccccc7)c6)cc5)cc5ccccc5c4o3)cc2)cc1 ; c1ccc(-c2ccc(-c3nc4ccc5c(-c6ccc(N(c7cccc(-c8ccccc8)c7)c7cccc(-c8ccccc8)c7)cc6)cccc5c4o3)cc2)cc1]

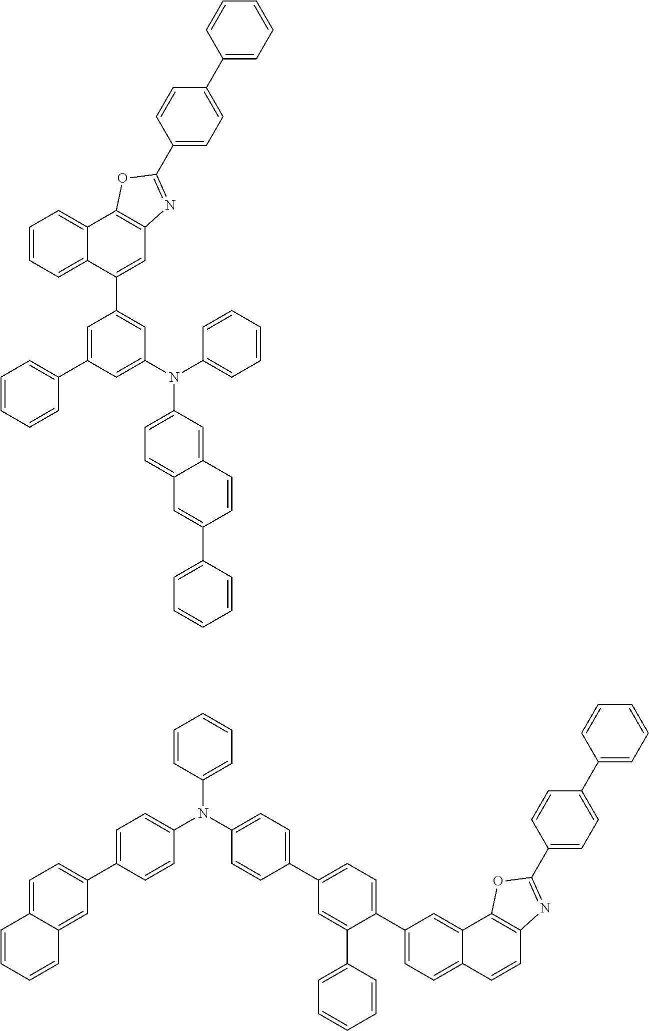 [SMILES: c1ccc(-c2ccc(-c3nc4cc(-c5cc(-c6ccccc6)cc(N(c6ccccc6)c6ccc7cc(-c8ccccc8)ccc7c6)c5)c5ccccc5c4o3)cc2)cc1 ; c1ccc(-c2ccc(-c3nc4ccc5ccc(-c6ccc(-c7ccc(N(c8ccccc8)c8ccc(-c9ccc%10ccccc%10c9)cc8)cc7)cc6-c6ccccc6)cc5c4o3)cc2)cc1]

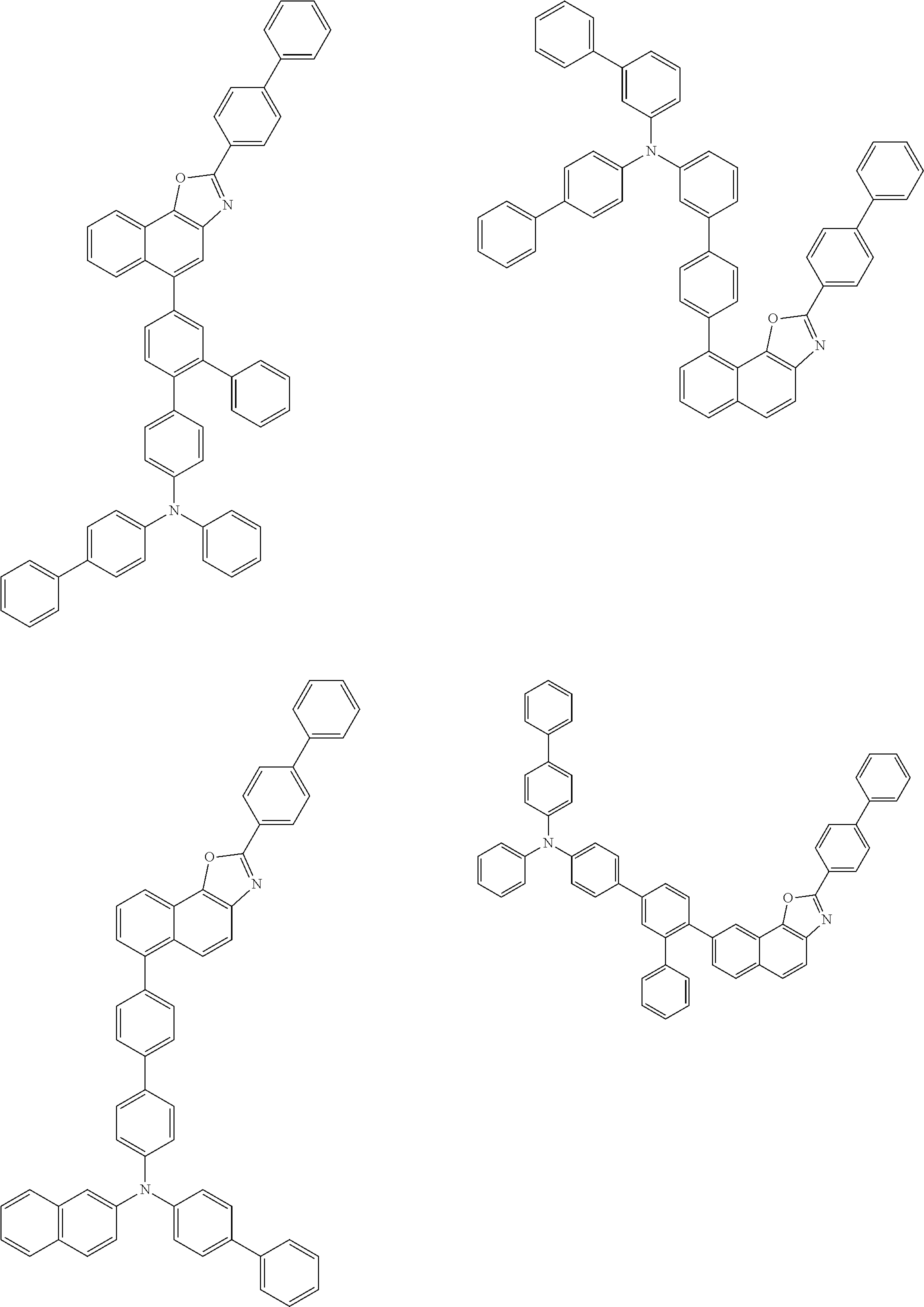 [SMILES: c1ccc(-c2ccc(-c3nc4cc(-c5ccc(-c6ccc(N(c7ccccc7)c7ccc(-c8ccccc8)cc7)cc6)c(-c6ccccc6)c5)c5ccccc5c4o3)cc2)cc1 ; c1ccc(-c2ccc(-c3nc4ccc5cccc(-c6ccc(-c7cccc(N(c8ccc(-c9ccccc9)cc8)c8cccc(-c9ccccc9)c8)c7)cc6)c5c4o3)cc2)cc1 ; c1ccc(-c2ccc(-c3nc4ccc5c(-c6ccc(-c7ccc(N(c8ccc(-c9ccccc9)cc8)c8ccc9ccccc9c8)cc7)cc6)cccc5c4o3)cc2)cc1 ; c1ccc(-c2ccc(-c3nc4ccc5ccc(-c6ccc(-c7ccc(N(c8ccccc8)c8ccc(-c9ccccc9)cc8)cc7)cc6-c6ccccc6)cc5c4o3)cc2)cc1]

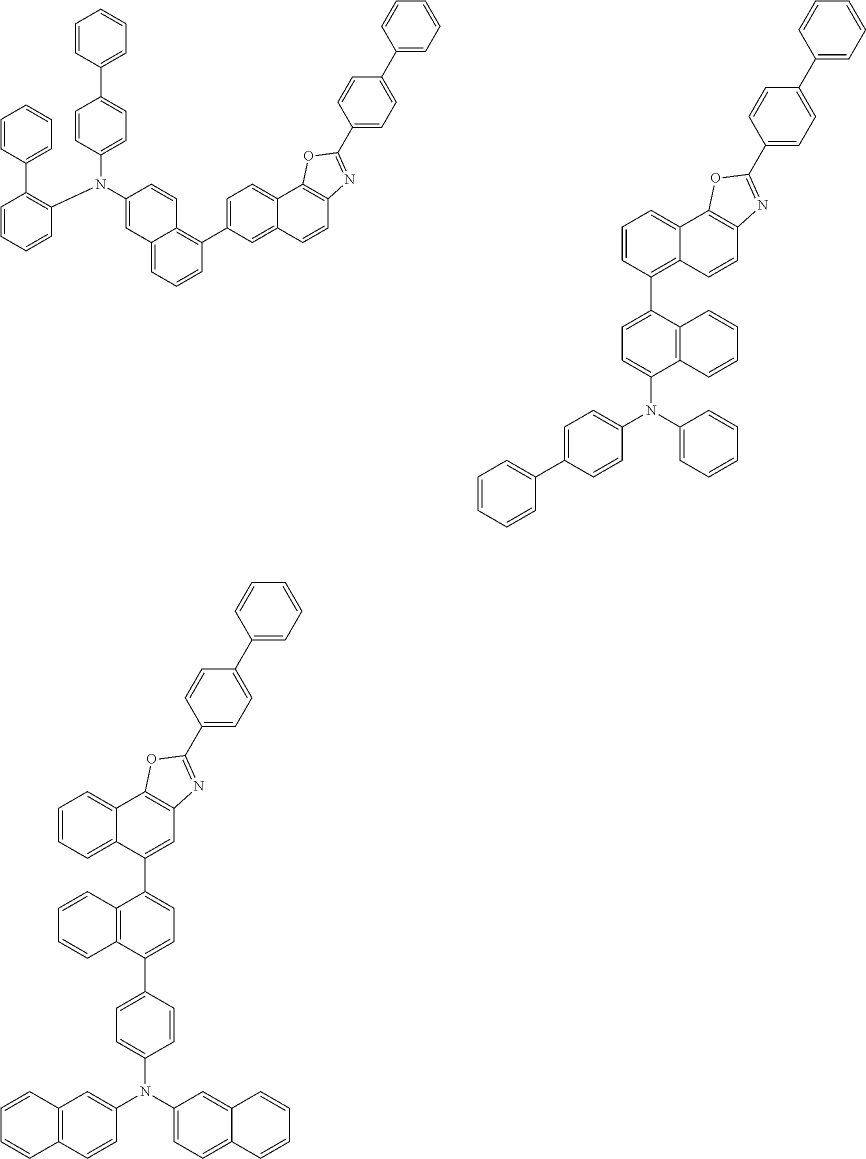 [SMILES: c1ccc(-c2ccc(-c3nc4ccc5cc(-c6cccc7cc(N(c8ccc(-c9ccccc9)cc8)c8ccccc8-c8ccccc8)ccc67)ccc5c4o3)cc2)cc1 ; c1ccc(-c2ccc(-c3nc4ccc5c(-c6ccc(N(c7ccccc7)c7ccc(-c8ccccc8)cc7)c7ccccc67)cccc5c4o3)cc2)cc1 ; c1ccc(-c2ccc(-c3nc4cc(-c5ccc(-c6ccc(N(c7ccc8ccccc8c7)c7ccc8ccccc8c7)cc6)c6ccccc56)c5ccccc5c4o3)cc2)cc1]

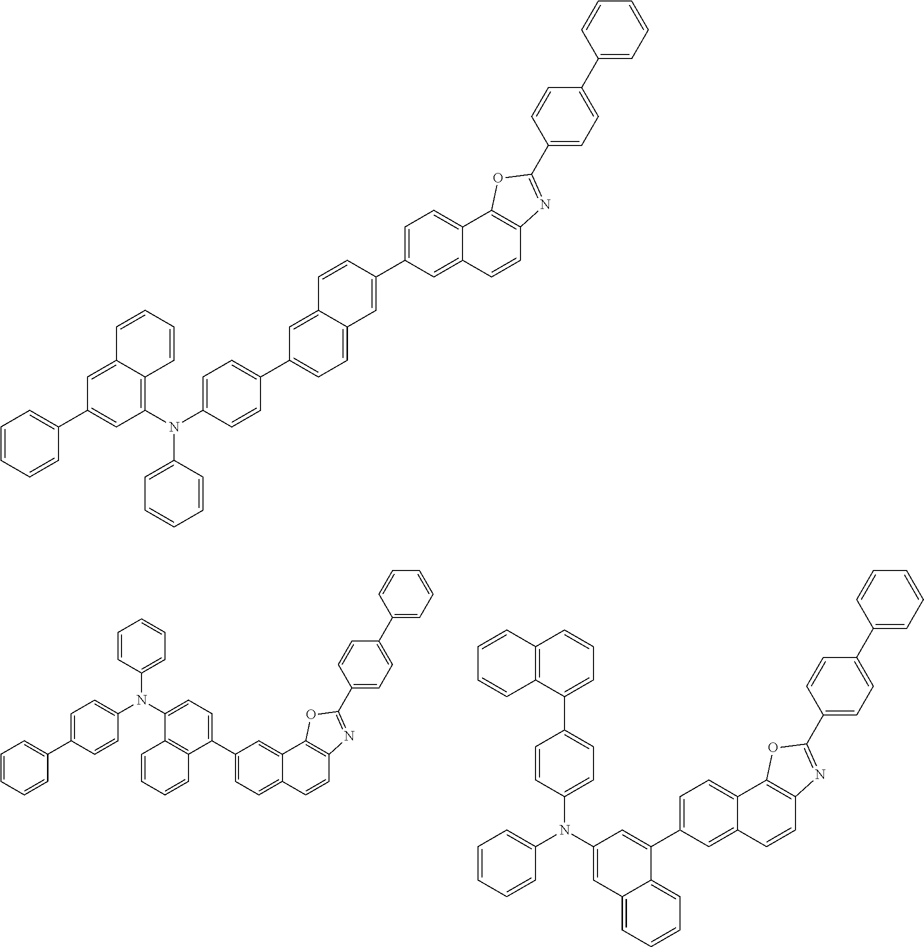 [SMILES: c1ccc(-c2ccc(-c3nc4ccc5cc(-c6ccc7cc(-c8ccc(N(c9ccccc9)c9cc(-c%10ccccc%10)cc%10ccccc9%10)cc8)ccc7c6)ccc5c4o3)cc2)cc1 ; c1ccc(-c2ccc(-c3nc4ccc5ccc(-c6ccc(N(c7ccccc7)c7ccc(-c8ccccc8)cc7)c7ccccc67)cc5c4o3)cc2)cc1 ; c1ccc(-c2ccc(-c3nc4ccc5cc(-c6cc(N(c7ccccc7)c7ccc(-c8cccc9ccccc89)cc7)cc7ccccc67)ccc5c4o3)cc2)cc1]

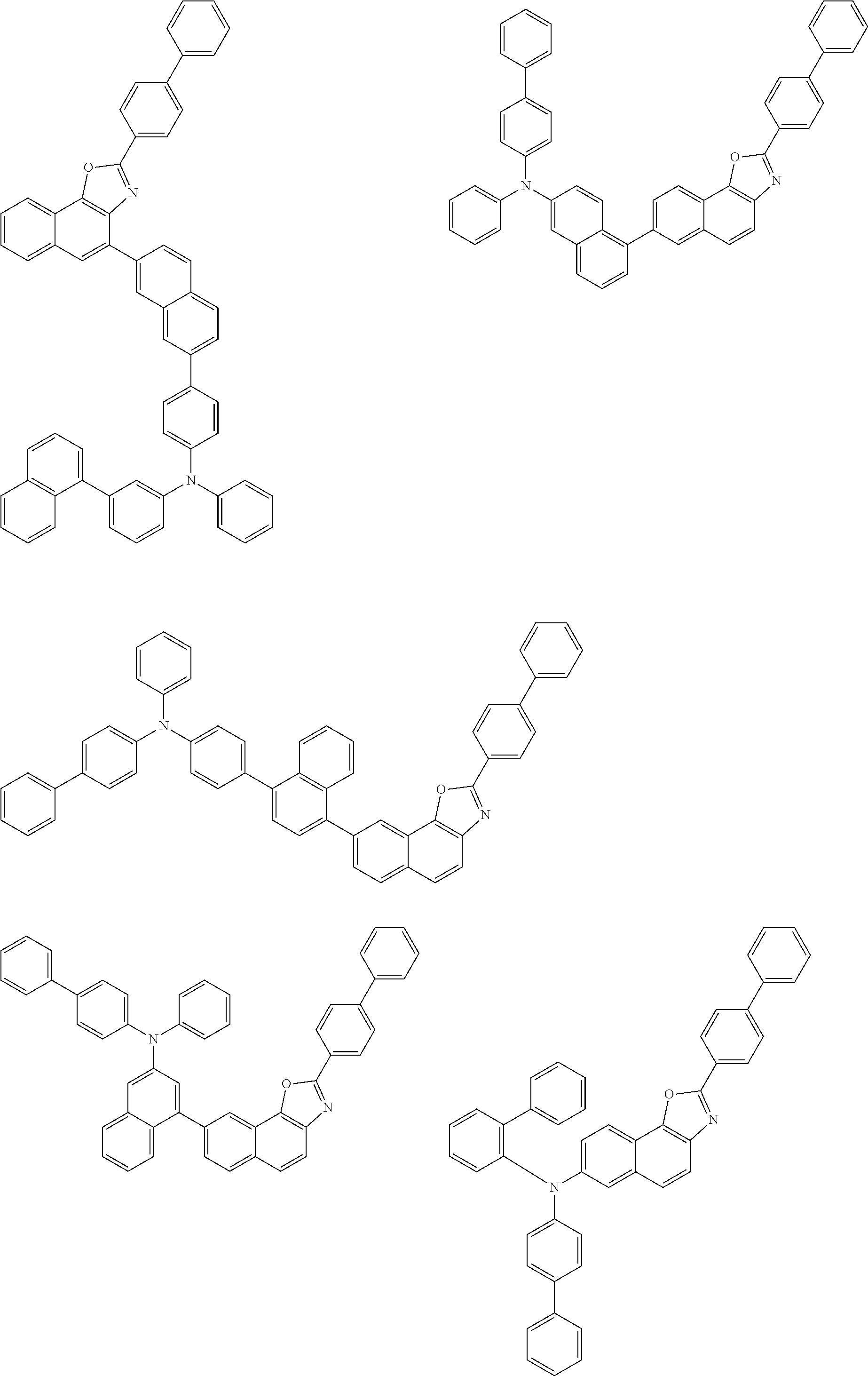 [SMILES: c1ccc(-c2ccc(-c3nc4c(-c5ccc6ccc(-c7ccc(N(c8ccccc8)c8cccc(-c9cccc%10ccccc9%10)c8)cc7)cc6c5)cc5ccccc5c4o3)cc2)cc1 ; c1ccc(-c2ccc(-c3nc4ccc5cc(-c6cccc7cc(N(c8ccccc8)c8ccc(-c9ccccc9)cc8)ccc67)ccc5c4o3)cc2)cc1 ; c1ccc(-c2ccc(-c3nc4ccc5ccc(-c6ccc(-c7ccc(N(c8ccccc8)c8ccc(-c9ccccc9)cc8)cc7)c7ccccc67)cc5c4o3)cc2)cc1 ; c1ccc(-c2ccc(-c3nc4ccc5ccc(-c6cc(N(c7ccccc7)c7ccc(-c8ccccc8)cc7)cc7ccccc67)cc5c4o3)cc2)cc1 ; c1ccc(-c2ccc(-c3nc4ccc5cc(N(c6ccc(-c7ccccc7)cc6)c6ccccc6-c6ccccc6)ccc5c4o3)cc2)cc1]

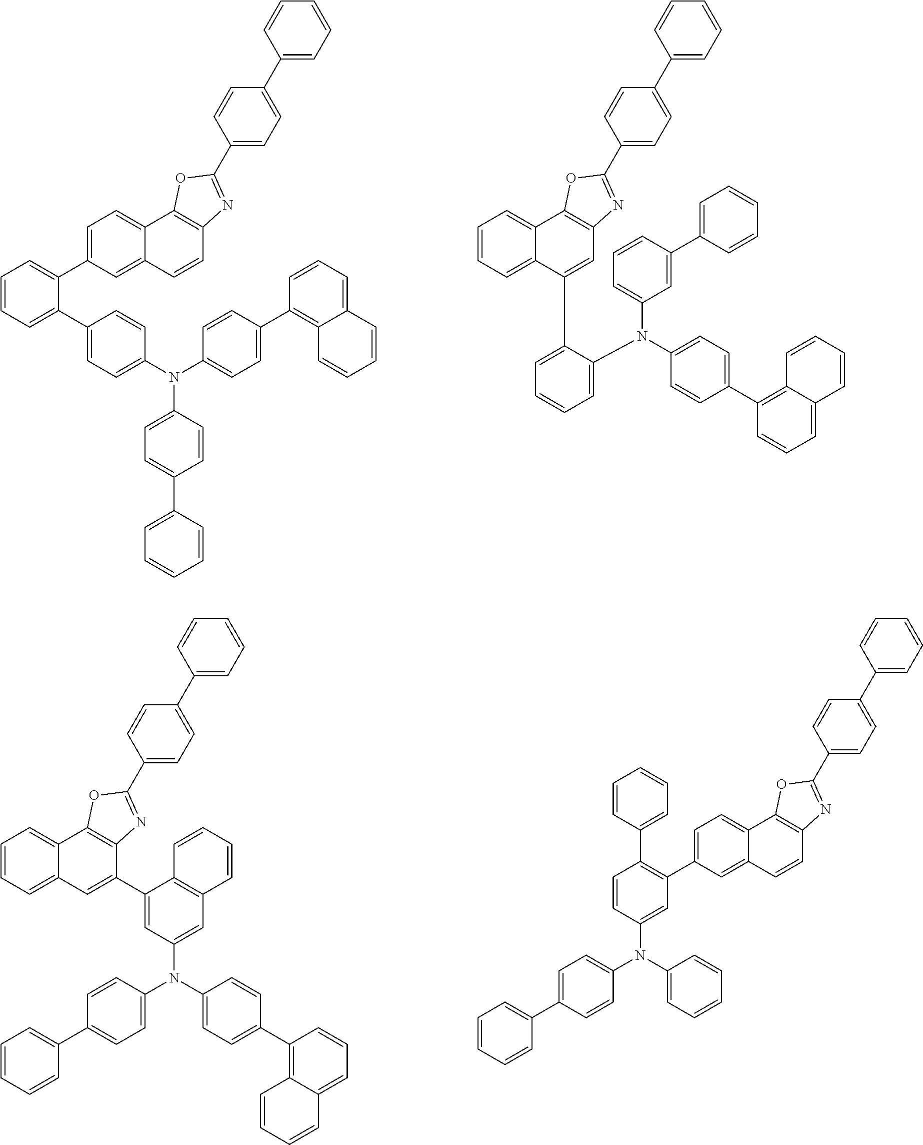

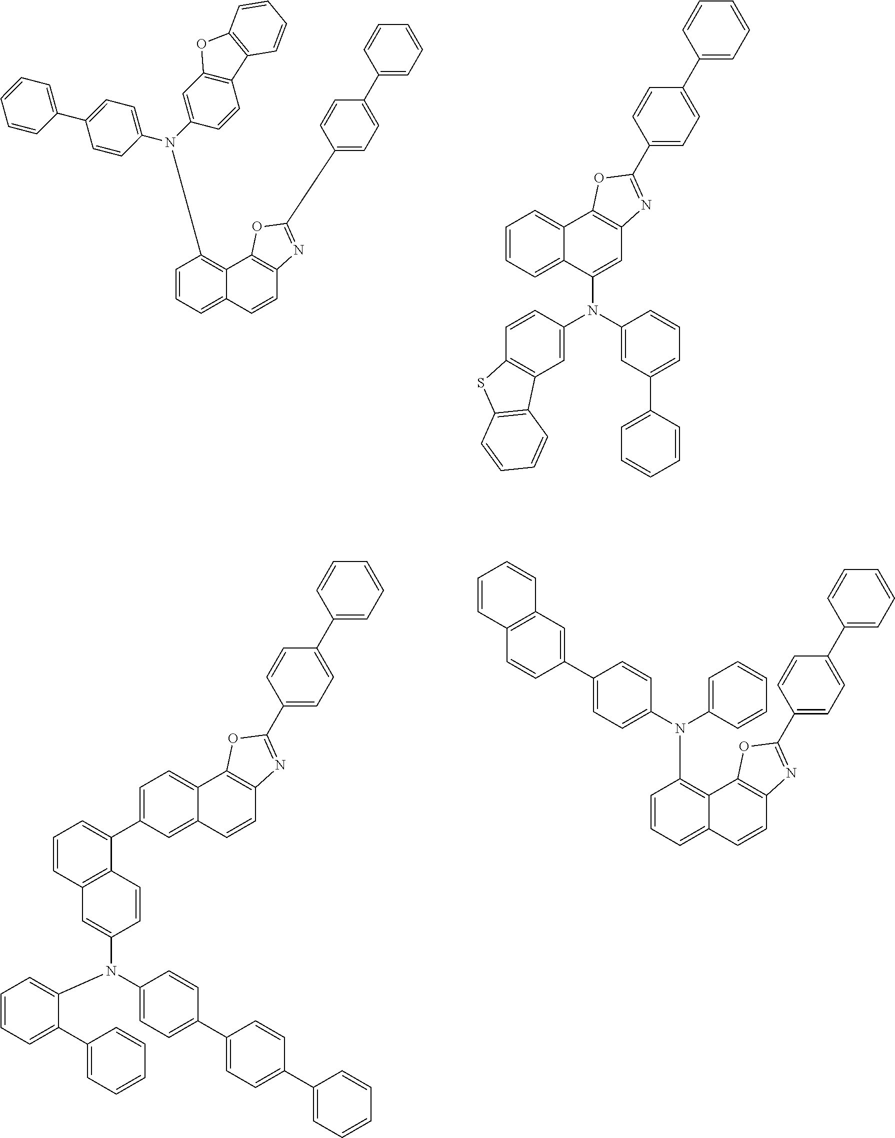 [SMILES: c1ccc(-c2ccc(-c3nc4ccc5cccc(N(c6ccc(-c7ccccc7)cc6)c6ccc7c(c6)oc6ccccc67)c5c4o3)cc2)cc1 ; c1ccc(-c2ccc(-c3nc4cc(N(c5cccc(-c6ccccc6)c5)c5ccc6sc7ccccc7c6c5)c5ccccc5c4o3)cc2)cc1 ; c1ccc(-c2ccc(-c3ccc(N(c4ccc5c(-c6ccc7c(ccc8nc(-c9ccc(-c%10ccccc%10)cc9)oc87)c6)cccc5c4)c4ccccc4-c4ccccc4)cc3)cc2)cc1 ; c1ccc(-c2ccc(-c3nc4ccc5cccc(N(c6ccccc6)c6ccc(-c7ccc8ccccc8c7)cc6)c5c4o3)cc2)cc1]

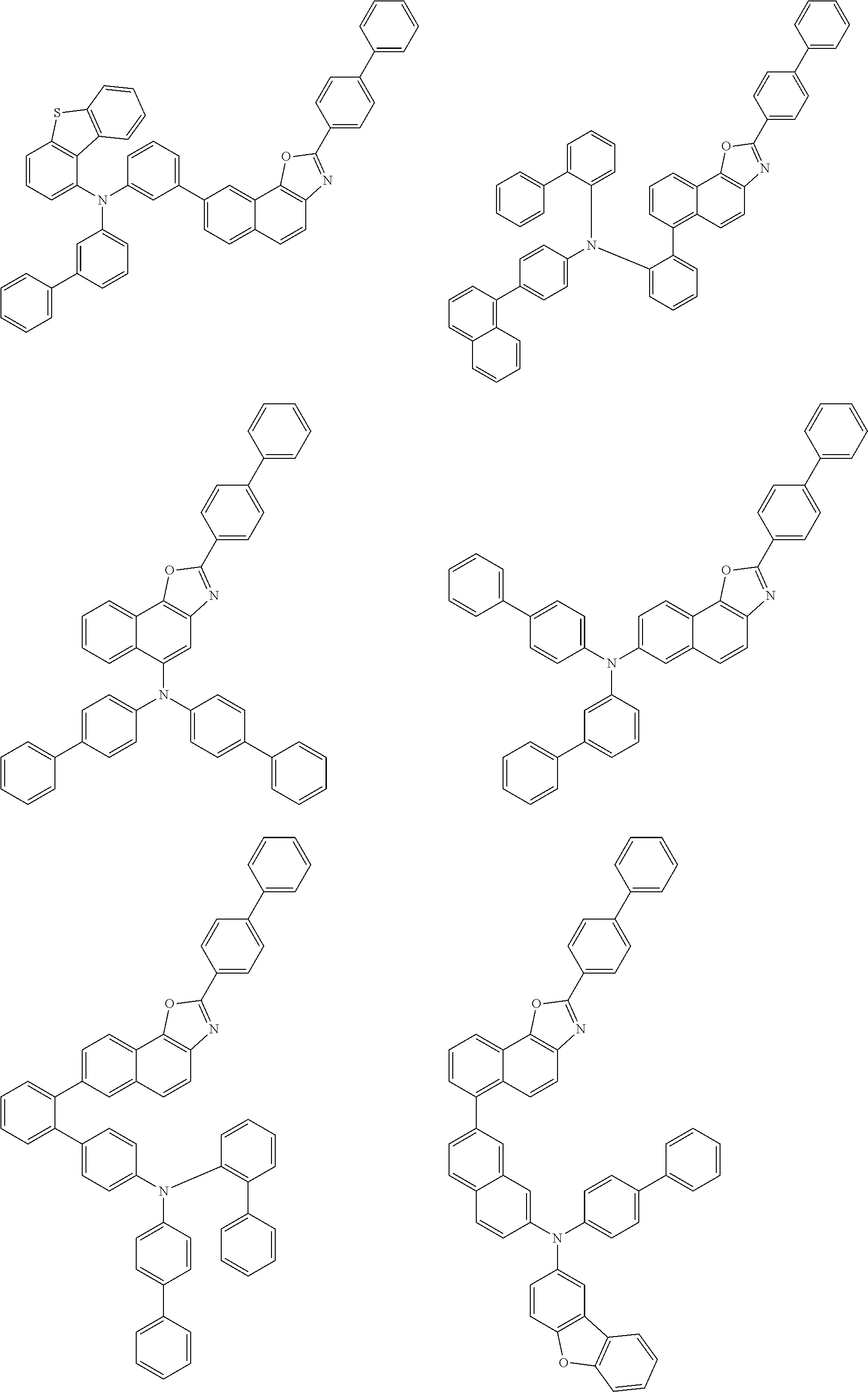

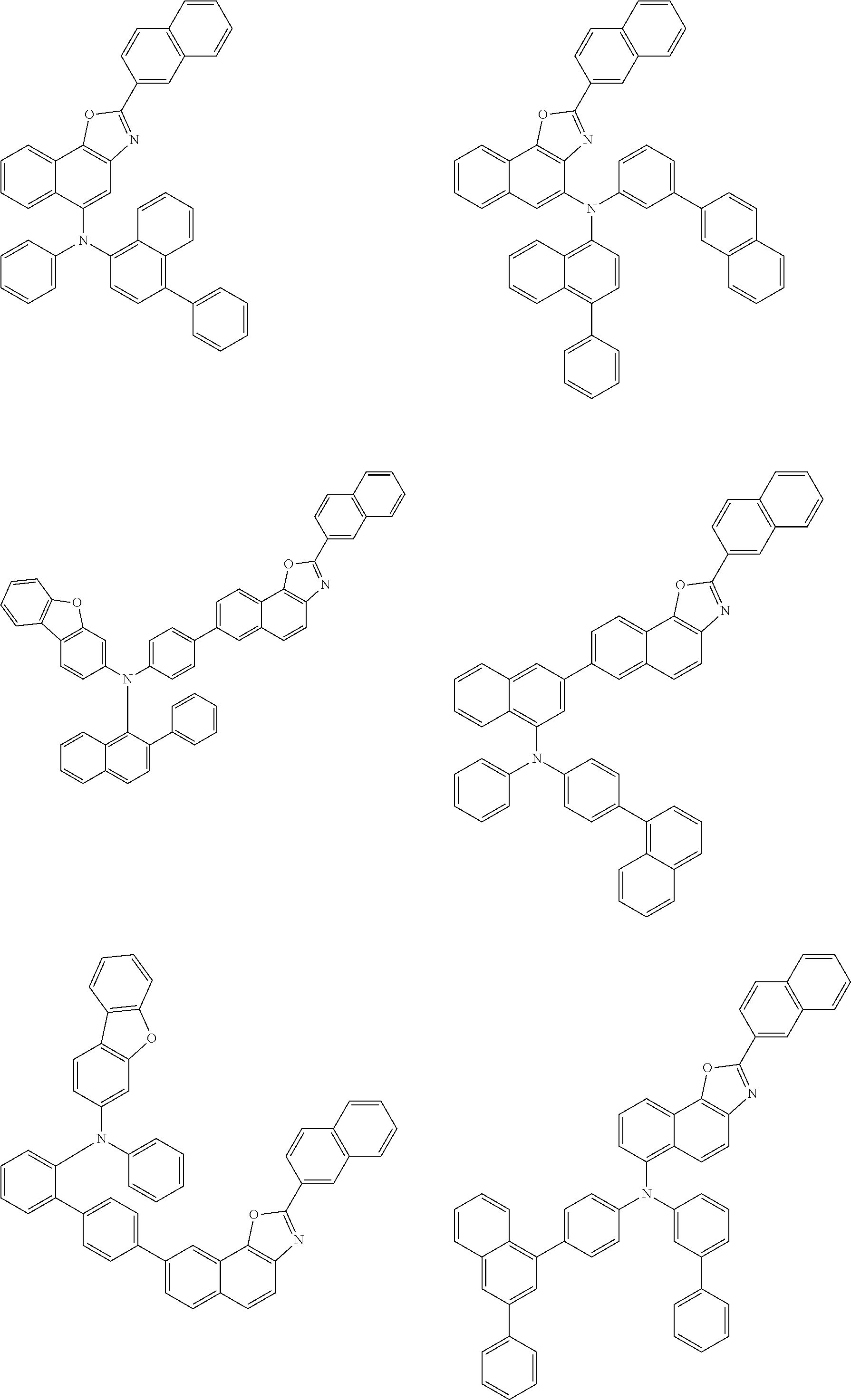

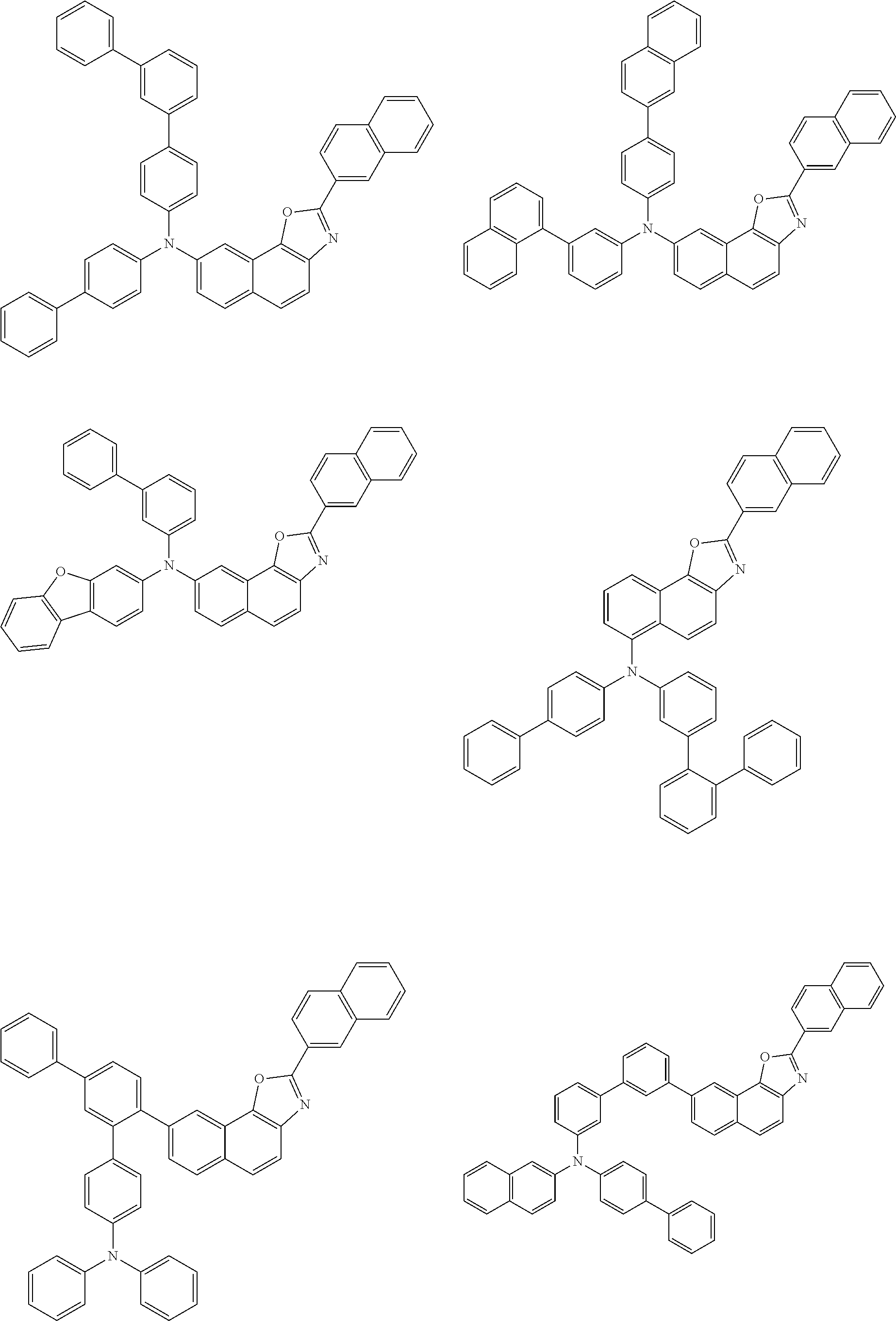

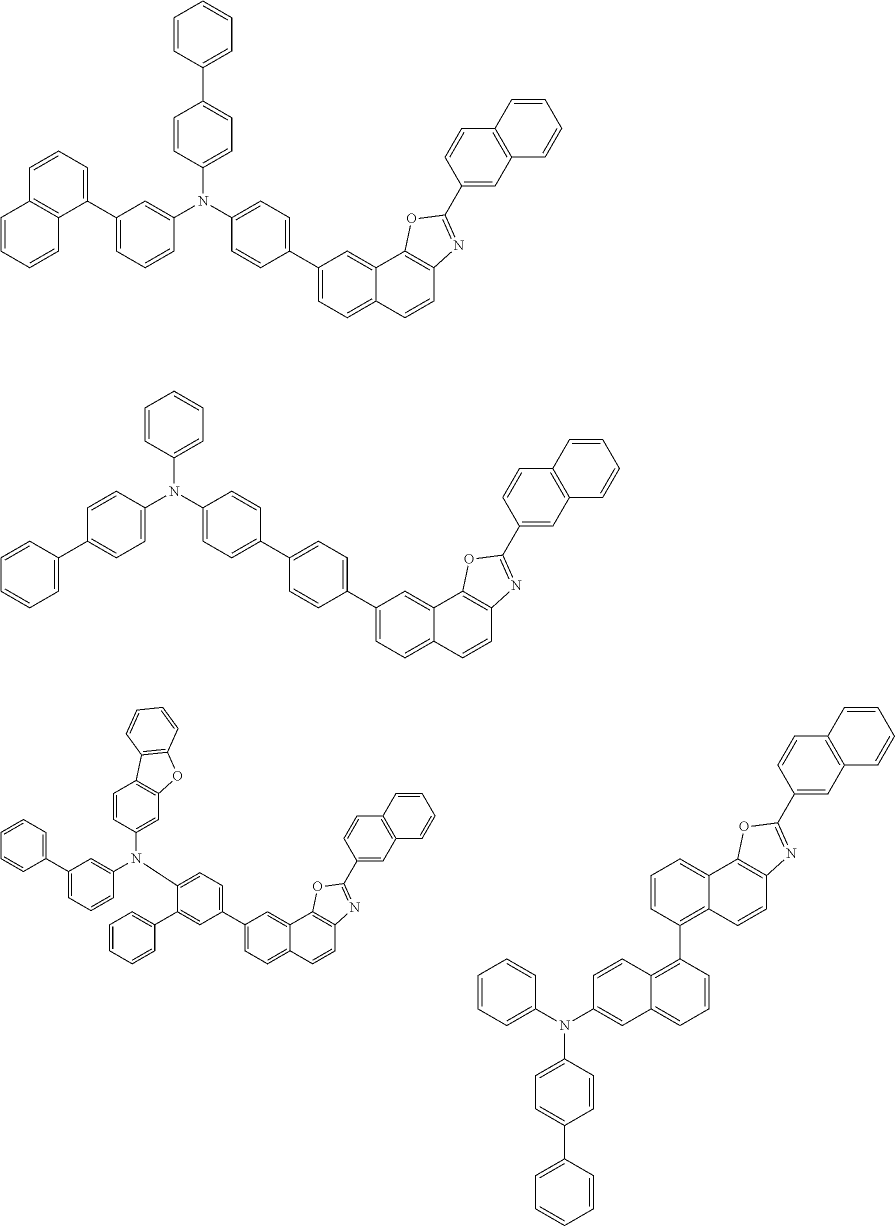 [SMILES: c1ccc(-c2ccc(N(c3ccc(-c4ccc5ccc6nc(-c7ccc8ccccc8c7)oc6c5c4)cc3)c3cccc(-c4cccc5ccccc45)c3)cc2)cc1 ; c1ccc(-c2ccc(N(c3ccccc3)c3ccc(-c4ccc(-c5ccc6ccc7nc(-c8ccc9ccccc9c8)oc7c6c5)cc4)cc3)cc2)cc1 ; c1ccc(-c2cccc(N(c3ccc4c(c3)oc3ccccc34)c3ccc(-c4ccc5ccc6nc(-c7ccc8ccccc8c7)oc6c5c4)cc3-c3ccccc3)c2)cc1 ; c1ccc(-c2ccc(N(c3ccccc3)c3ccc4c(-c5cccc6c5ccc5nc(-c7ccc8ccccc8c7)oc56)cccc4c3)cc2)cc1]

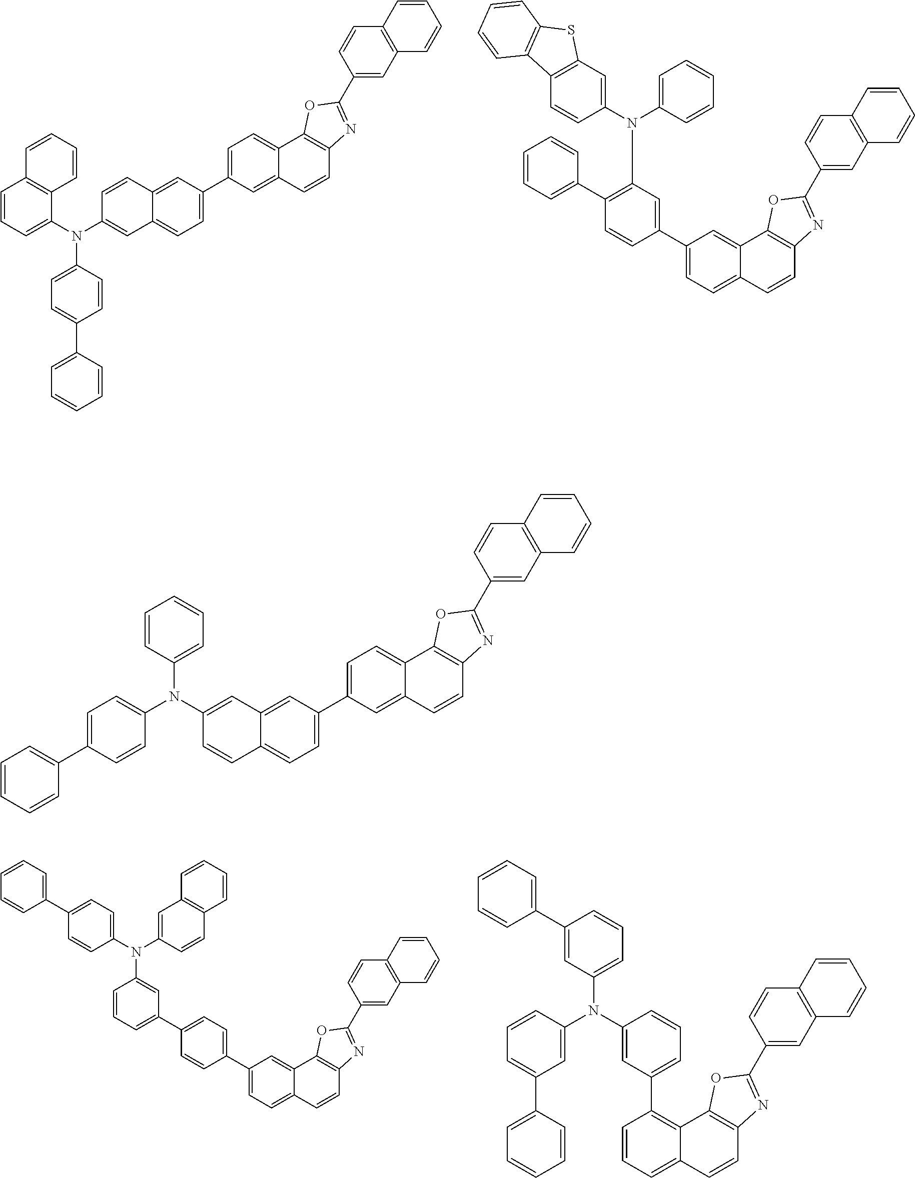

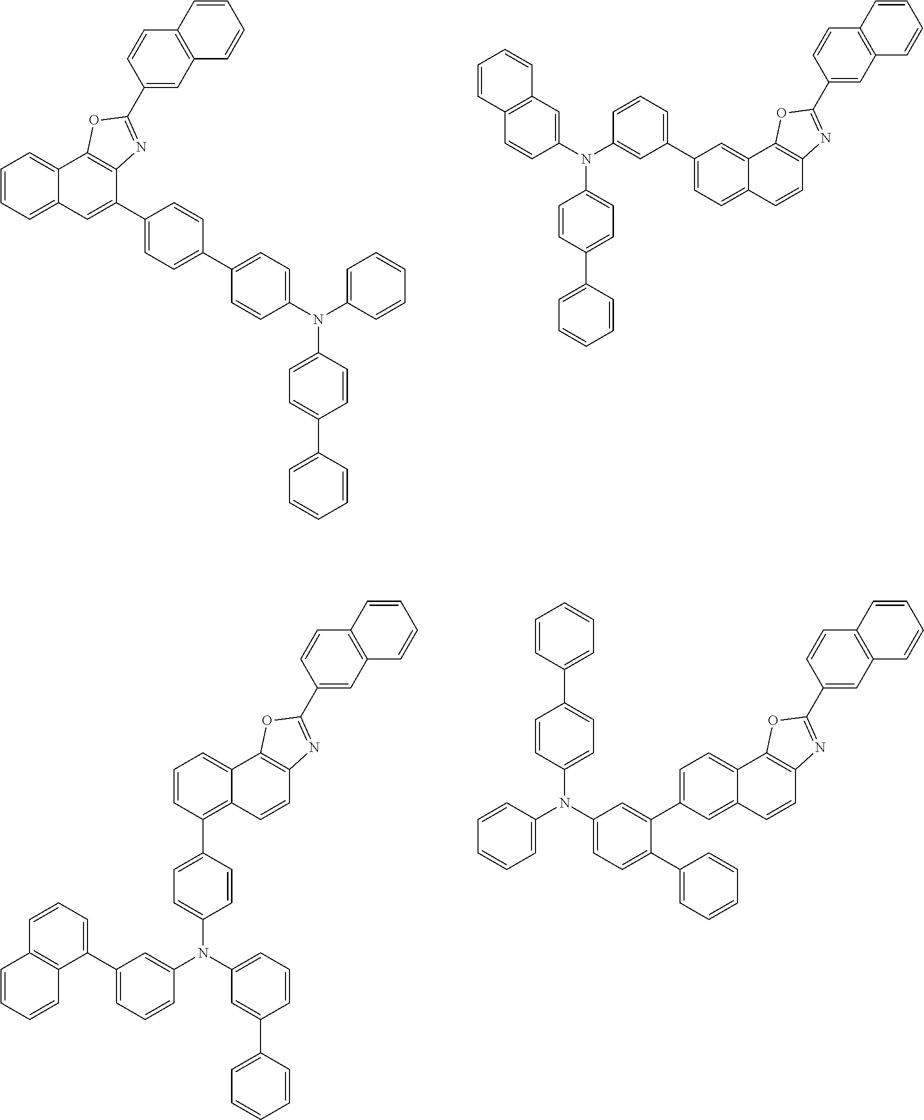 [SMILES: c1ccc(-c2ccc(N(c3ccccc3)c3ccc(-c4ccc(-c5cc6ccccc6c6oc(-c7ccc8ccccc8c7)nc56)cc4)cc3)cc2)cc1 ; c1ccc(-c2ccc(N(c3cccc(-c4ccc5ccc6nc(-c7ccc8ccccc8c7)oc6c5c4)c3)c3ccc4ccccc4c3)cc2)cc1 ; c1ccc(-c2cccc(N(c3ccc(-c4cccc5c4ccc4nc(-c6ccc7ccccc7c6)oc45)cc3)c3cccc(-c4cccc5ccccc45)c3)c2)cc1 ; c1ccc(-c2ccc(N(c3ccccc3)c3ccc(-c4ccccc4)c(-c4ccc5c(ccc6nc(-c7ccc8ccccc8c7)oc65)c4)c3)cc2)cc1]

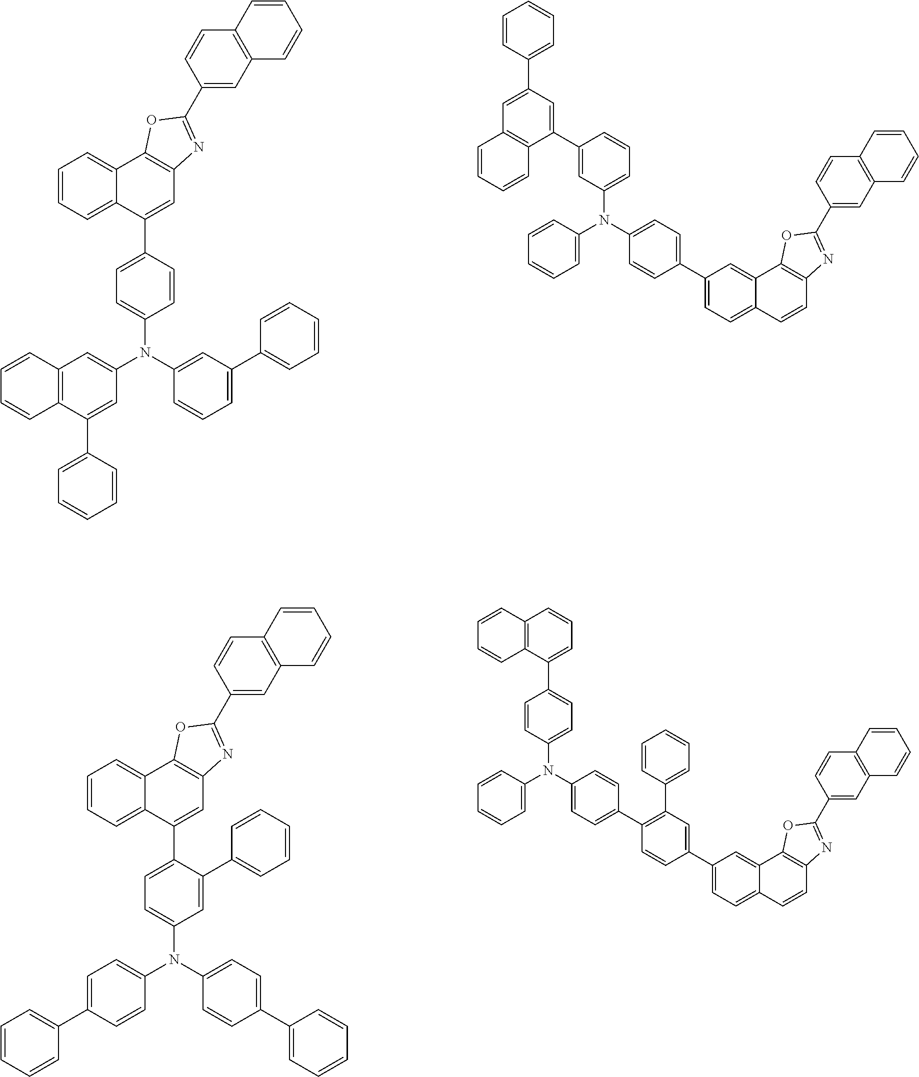 [SMILES: c1ccc(-c2cccc(N(c3ccc(-c4cc5nc(-c6ccc7ccccc7c6)oc5c5ccccc45)cc3)c3cc(-c4ccccc4)c4ccccc4c3)c2)cc1 ; c1ccc(-c2cc(-c3cccc(N(c4ccccc4)c4ccc(-c5ccc6ccc7nc(-c8ccc9ccccc9c8)oc7c6c5)cc4)c3)c3ccccc3c2)cc1 ; c1ccc(-c2ccc(N(c3ccc(-c4ccccc4)cc3)c3ccc(-c4cc5nc(-c6ccc7ccccc7c6)oc5c5ccccc45)c(-c4ccccc4)c3)cc2)cc1 ; c1ccc(-c2cc(-c3ccc4ccc5nc(-c6ccc7ccccc7c6)oc5c4c3)ccc2-c2ccc(N(c3ccccc3)c3ccc(-c4cccc5ccccc45)cc3)cc2)cc1]

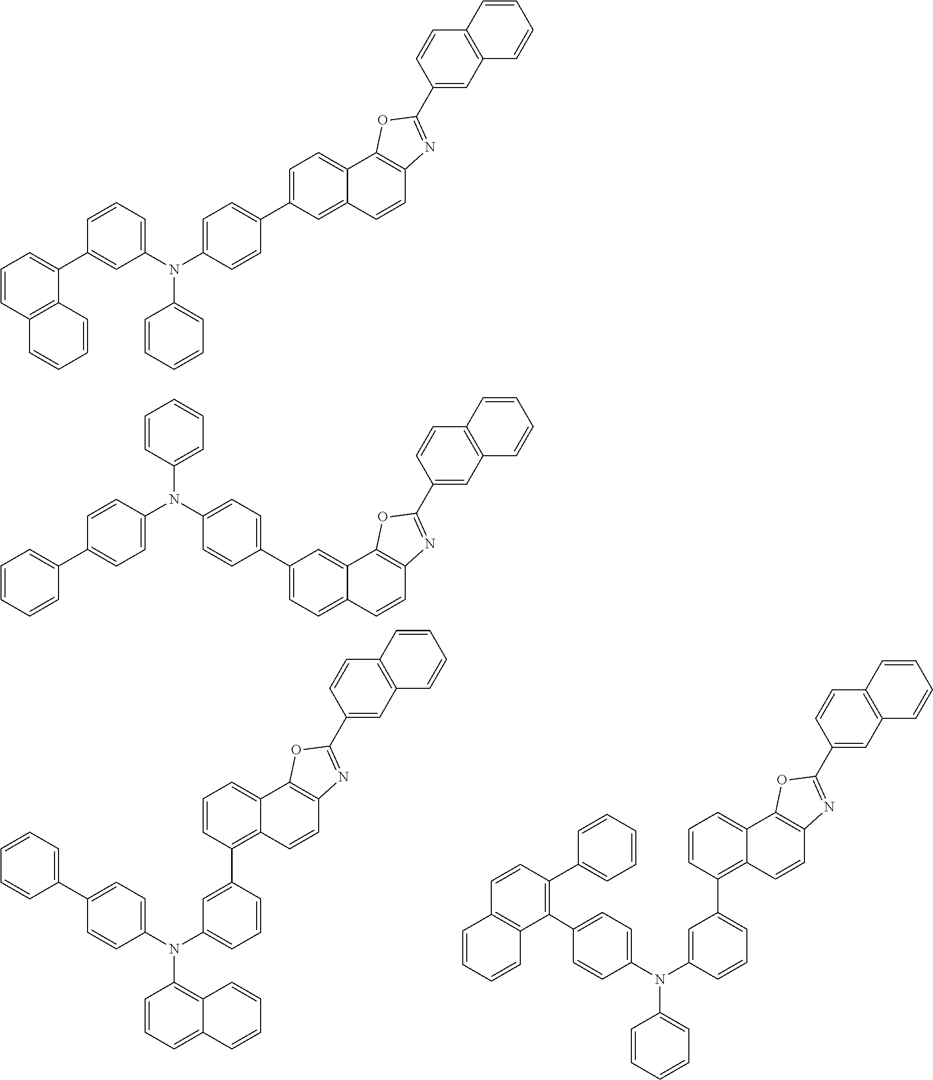

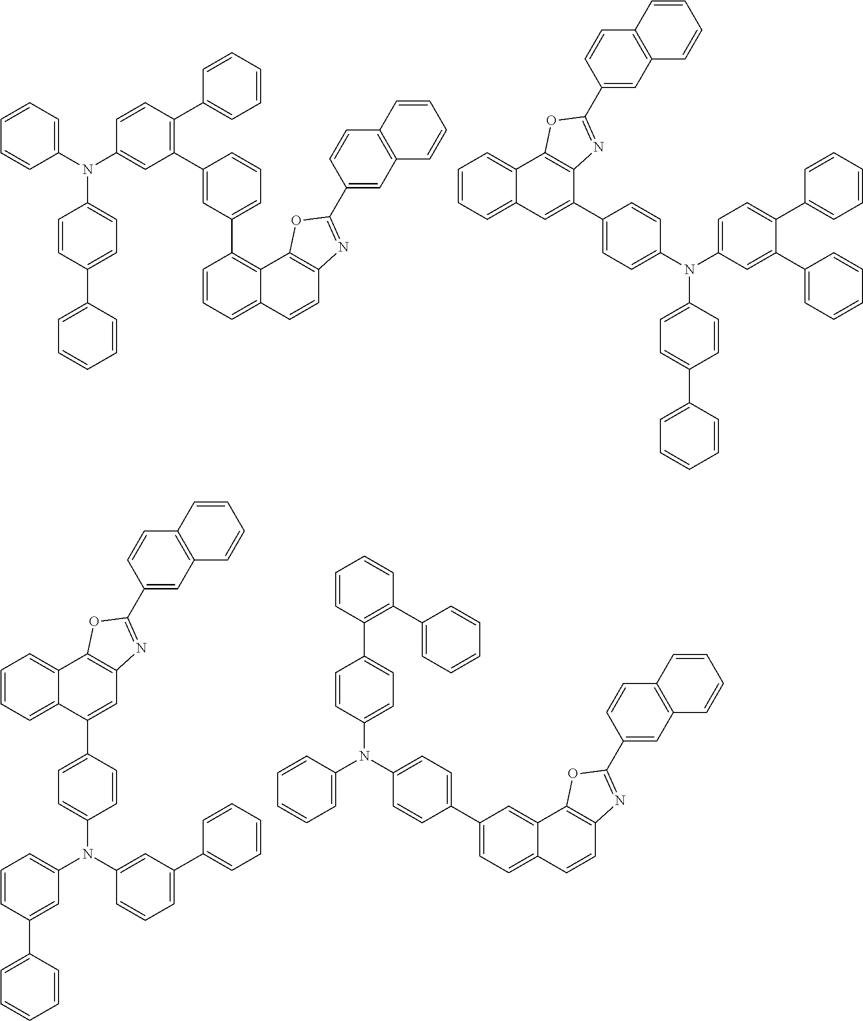

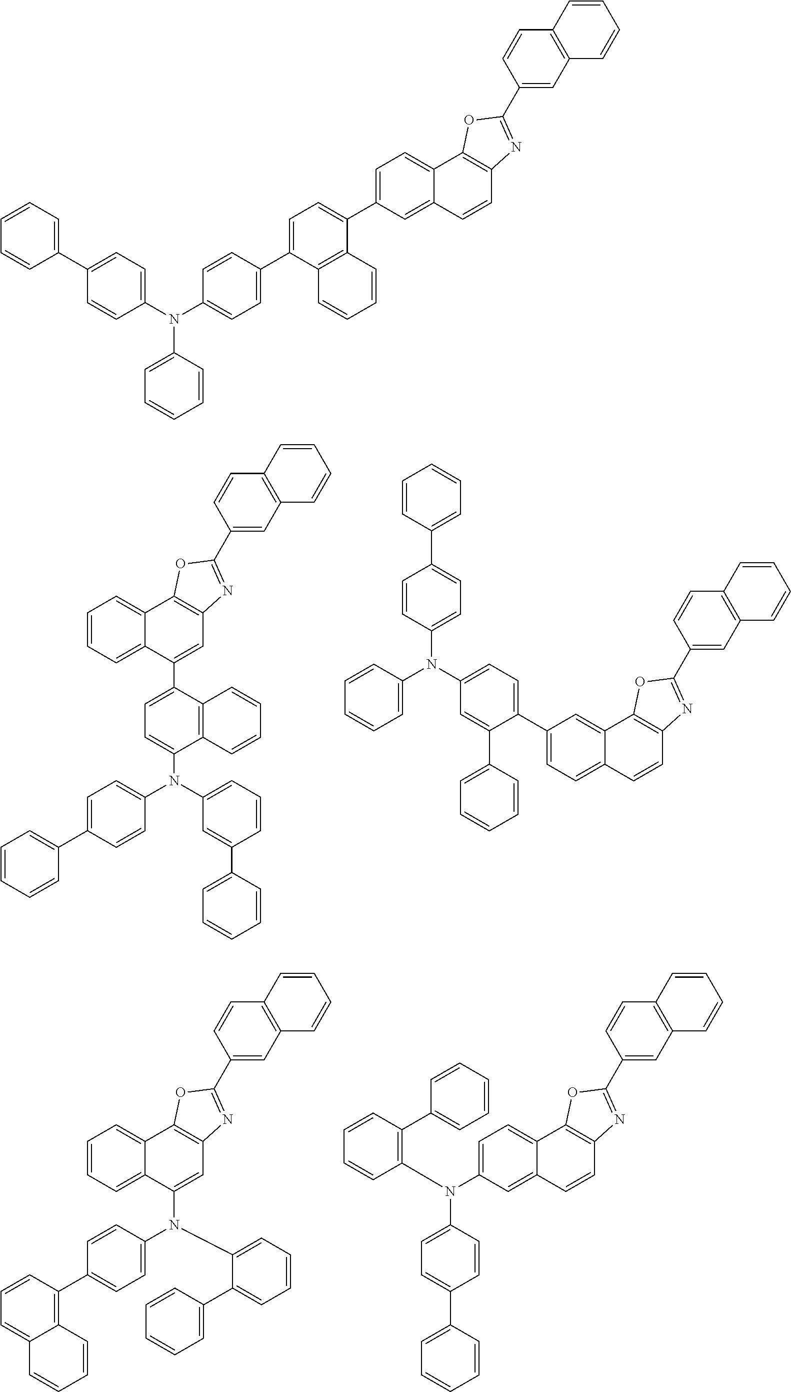 [SMILES: c1ccc(-c2ccc(N(c3ccccc3)c3ccc(-c4ccc(-c5ccc6c(ccc7nc(-c8ccc9ccccc9c8)oc76)c5)c5ccccc45)cc3)cc2)cc1 ; c1ccc(-c2ccc(N(c3cccc(-c4ccccc4)c3)c3ccc(-c4cc5nc(-c6ccc7ccccc7c6)oc5c5ccccc45)c4ccccc34)cc2)cc1 ; c1ccc(-c2ccc(N(c3ccccc3)c3ccc(-c4ccc5ccc6nc(-c7ccc8ccccc8c7)oc6c5c4)c(-c4ccccc4)c3)cc2)cc1 ; c1ccc(-c2ccccc2N(c2ccc(-c3cccc4ccccc34)cc2)c2cc3nc(-c4ccc5ccccc5c4)oc3c3ccccc23)cc1 ; c1ccc(-c2ccc(N(c3ccc4c(ccc5nc(-c6ccc7ccccc7c6)oc54)c3)c3ccccc3-c3ccccc3)cc2)cc1]

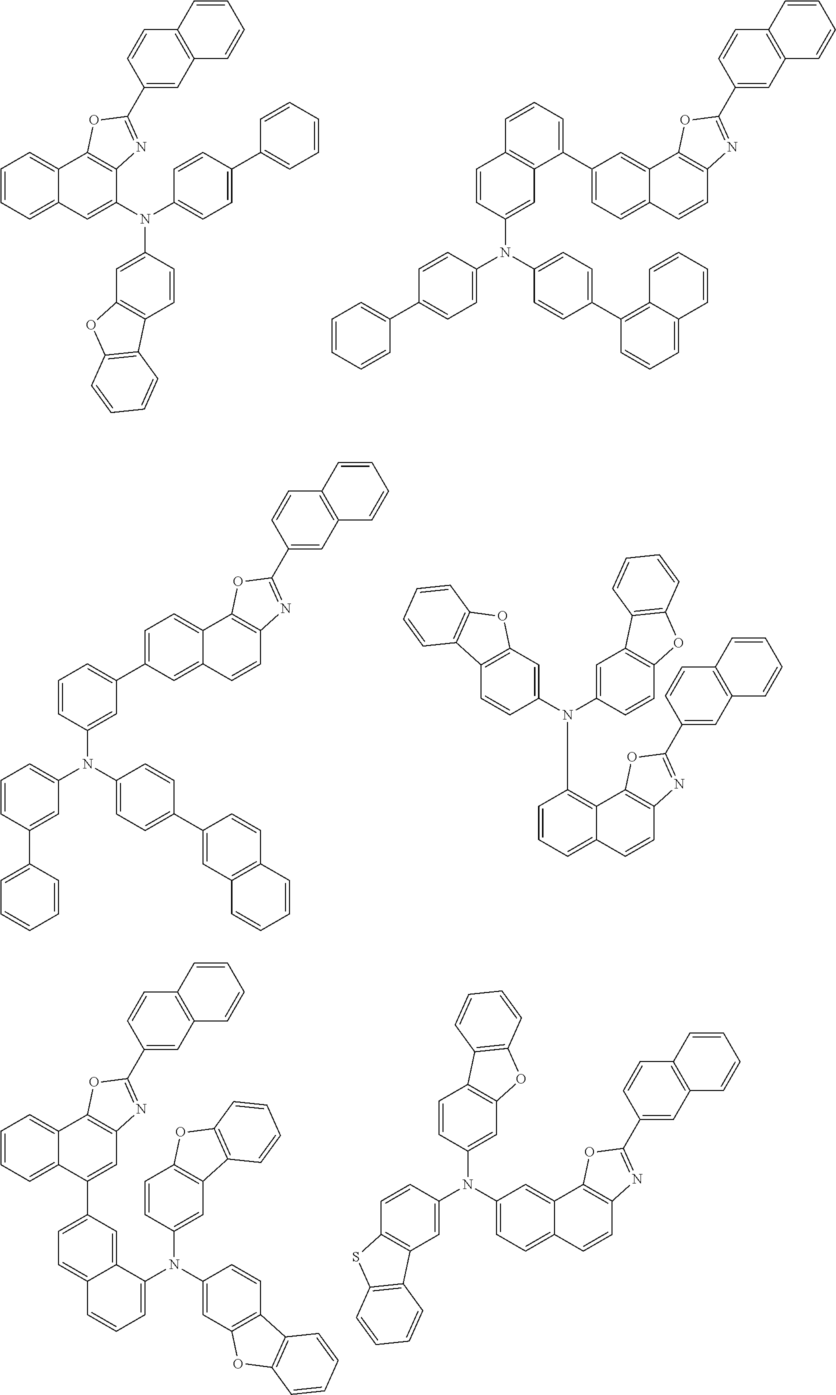

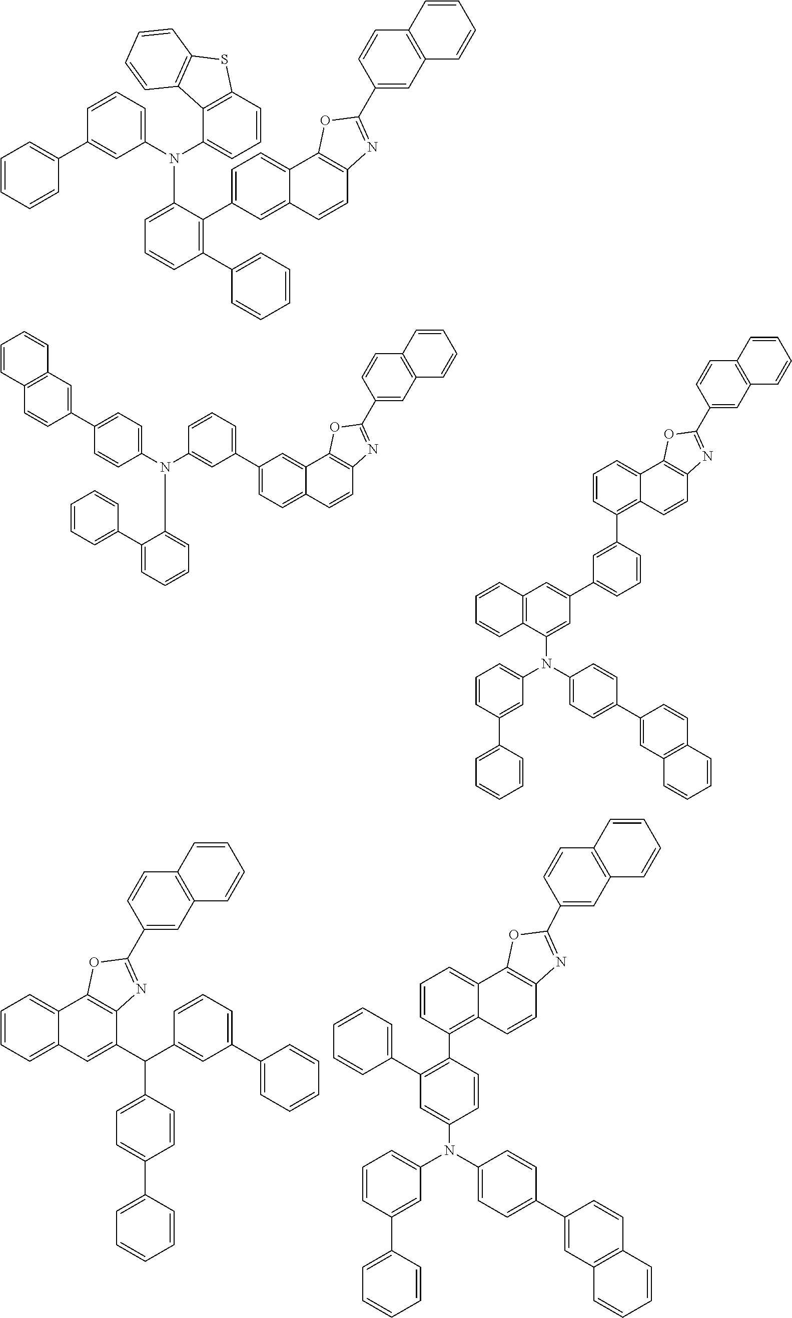

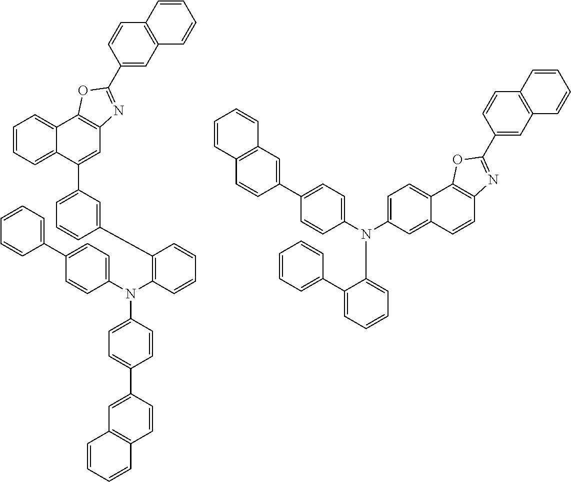 [SMILES: c1ccc(-c2ccc(N(c3ccc(-c4ccc5ccccc5c4)cc3)c3ccccc3-c3cccc(-c4cc5nc(-c6ccc7ccccc7c6)oc5c5ccccc45)c3)cc2)cc1 ; c1ccc(-c2ccccc2N(c2ccc(-c3ccc4ccccc4c3)cc2)c2ccc3c(ccc4nc(-c5ccc6ccccc6c5)oc43)c2)cc1]

In addition, the present disclosure provides a method for preparing a compound represented by the Chemical Formula 1 in which R₁ is the Chemical Formula 3 as shown in Reaction Scheme 1 below, and the other compounds represented by the Chemical Formulae 1 and 2 may also be prepared in a similar manner.

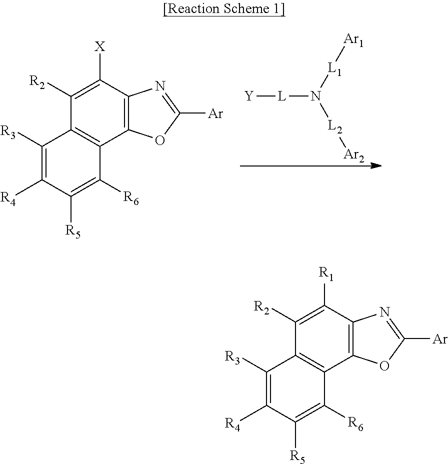

in the Reaction Scheme 1, definitions of other substituents except for X and Y are the same as defined above, and X is halogen, preferably bromo, or chloro. Y is hydrogen when L is a single bond, and —B(OH)₂ when L is not a single bond. The Reaction Scheme 1 is an amine substitution reaction or a Suzuki coupling reaction, and preferably performed in the presence of a palladium catalyst and a base. In addition, the reactive group for each reaction may be appropriately changed, and the preparation method may be more specifically described in Preparation Examples described below.

In addition, according to another aspect of the present disclosure, there is provided an organic light emitting device including the above-mentioned compound represented by the Chemical Formula 1 or Chemical Formula 2. As an example, there is provided an organic light emitting device including: a first electrode; a second electrode disposed to face the first electrode; and an organic material layer including one or more layers between the first electrode and the second electrode, wherein one or more layers of the organic material layer include the compound represented by the Chemical Formula 1 or 2.

The organic material layer of the organic light emitting device of the present disclosure may have a single-layer structure, or it may have a multilayered structure in which two or more organic layers are stacked. For example, the organic light emitting device of the present disclosure may have a structure including a hole injection layer, a hole transport layer, an electron blocking layer, a light emitting layer, a hole blocking layer, an electron transport layer, an electron injection layer, and the like as the organic material layer. However, the structure of the organic light emitting device is not limited thereto, and it may include a smaller number of organic layers.

The organic material layer may include a light emitting layer, and the light emitting layer may include a compound represented by the Chemical Formula 1 or Chemical Formula 2. In particular, the compound according to the present disclosure can be used as a dopant in the light emitting layer.

In addition, the organic material layer may include an electron transport layer or an electron injection layer, and the electron transport layer or the electron injection layer may include a compound represented by the Chemical Formula 1 or Chemical Formula 2.

In addition, the organic material layer may include an electron transport and injection layer, and the electron transport and injection layer may include a compound represented by the Chemical Formula 1 or Chemical Formula 2.

In addition, the organic material layer may include a light emitting layer and an electron transport layer, and the electron transport layer may include a compound represented by the Chemical Formula 1 or Chemical Formula 2.

In addition, the organic material layer may include an electron blocking layer, and the electron blocking layer may include a compound represented by the Chemical Formula 1 or Chemical Formula 2.

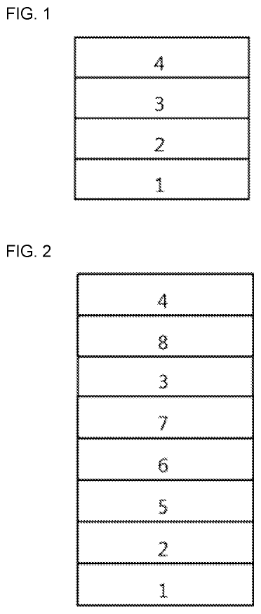

Further, the organic light emitting device according to the present disclosure may be a normal type organic light emitting device in which an anode, one or more layers of an organic material layer and a cathode are sequentially stacked on a substrate. Further, the organic light emitting device according to the present disclosure may be an inverted type organic light emitting device in which a cathode, one or more layers of an organic material layer and an anode are sequentially stacked on a substrate. For example, the structure of an organic light emitting device according to an embodiment of the present disclosure is illustrated in FIGS. 1 and 2.

FIG. 1 shows an example of an organic light emitting device including a substrate 1, an anode 2, a light emitting layer 3, and a cathode 4. In such a structure, the compound represented by the Chemical Formula 1 or Chemical Formula 2 may be included in the light emitting layer.

FIG. 2 shows an example of an organic light emitting device including a substrate 1, an anode 2, a hole injection layer 5, a hole transport layer 6, an electron blocking layer 7, a light emitting layer 3, an electron transport layer 8, and a cathode 4. In such a structure, the compound represented by the Chemical Formula 1 or Chemical Formula 2 may be included in at least one of the hole injection layer, the hole transport layer, the electron blocking layer, the light emitting layer, and the electron transport layer.

The organic light emitting device according to the present disclosure may be manufactured using materials and methods known in the art, except that one or more layers of the organic material layer include the compound represented by Chemical Formula 1 or Chemical Formula 2. Moreover, when the organic light emitting device includes the organic material layer comprising a plurality of organic layers, the organic layers may be formed of the same material or different materials.

For example, the organic light emitting device according to the present disclosure can be manufactured by sequentially stacking a first electrode, an organic material layer and a second electrode on a substrate. In this case, the organic light emitting device may be manufactured by depositing a metal, metal oxides having conductivity, or an alloy thereof on the substrate using a PVD (physical vapor deposition) method such as a sputtering method or an e-beam evaporation method to form an anode, forming an organic material layer including the hole injection layer, the hole transport layer, the light emitting layer and the electron transport layer thereon, and then depositing a material that can be used as the cathode thereon. In addition to such a method, the organic light emitting device may be manufactured by sequentially depositing a cathode material, an organic material layer and an anode material on a substrate.

Further, the compound represented by Chemical Formula 1 or Chemical Formula 2 may be formed into an organic material layer by a solution coating method as well as a vacuum deposition method at the time of manufacturing an organic light emitting device. Herein, the solution coating method means a spin coating, a dip coating, a doctor blading, an inkjet printing, a screen printing, a spray method, a roll coating, or the like, but is not limited thereto.

In addition to such a method, the organic light emitting device may be manufactured by sequentially depositing a cathode material, an organic material layer and an anode material on a substrate (International Publication WO2003/012890). However, the manufacturing method is not limited thereto.

For example, the first electrode is an anode and the second electrode is a cathode, or alternatively, the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SnO₂:Sb; conductive polymers such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

The hole injection layer is a layer for injecting holes from the electrode, and the hole injection material is preferably a compound which has a capability of transporting the holes, thus has a hole injecting effect in the anode and an excellent hole-injecting effect to the light emitting layer or the light emitting material, prevents excitons produced in the light emitting layer from moving to an electron injection layer or the electron injection material, and is excellent in the ability to form a thin film. It is preferable that a HOMO (highest occupied molecular orbital) of the hole injection material is between the work function of the anode material and a HOMO of a peripheral organic material layer. Specific examples of the hole injection material include metal porphyrine, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

In addition, the hole transport layer is a layer that receives holes from a hole injection layer and transports the holes to the light emitting layer. The hole transport material is suitably a material having large mobility to the holes, which may receive holes from the anode or the hole injection layer and transfer the holes to the light emitting layer. Specific examples thereof include an arylamine-based organic material, a conductive polymer, a block copolymer in which a conjugate portion and a non-conjugate portion are present together, and the like, but are not limited thereto.

The light emitting material is suitably a material capable of emitting light in a visible ray region by receiving holes and electrons from the hole transport layer and the electron transport layer, respectively, to combine them, and having good quantum efficiency to fluorescence or phosphorescence. Specific examples thereof include 8-hydroxy-quinoline aluminum complex (Alq₃); a carbazole-based compound; a dimerized styryl compound; BAlq; a 10-hydroxybenzo quinoline-metal compound; a benzoxazole-, benzthiazole- and benzimidazole-based compound; a poly(p-phenylenevinylene) (PPV)-based polymer; a spiro compound; polyfluorene, rubrene, and the like, but are not limited thereto.

In addition, the light emitting layer may include a host material and a dopant material. The host material may be a fused aromatic ring derivative or a heterocycle-containing compound. Specific examples of the fused aromatic ring derivatives include anthracene derivatives, pyrene derivatives, naphthalene derivatives, pentacene derivatives, phenanthrene compounds, fluoranthene compounds, and the like. Examples of the heterocyclic-containing compounds include carbazole derivatives, dibenzofuran derivatives, ladder-type furan compounds, pyrimidine derivatives, and the like, but are not limited thereto.

The dopant material includes an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene and the like, which have an arylamino group. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto.

The electron transport layer is a layer which receives electrons from an electron injection layer and transports the electrons to a light emitting layer, and an electron transport material used is suitably a material which can receive electrons well from a cathode and transfer the electrons to a light emitting layer and has large mobility for electrons. Specifically, examples thereof may include an Al complex of 8-hydroxyquinoline; a complex including Alq₃; an organic radical compound; a hydroxyflavone-metal complex, and the like, but are not limited thereto. The electron transport layer may be used with any desired cathode material, as used according to the related art. In particular, appropriate examples of the cathode material are a typical material which has a low work function, followed by an aluminum layer or a silver layer. Specific examples thereof include cesium, barium, calcium, ytterbium, and samarium, in each case followed by an aluminum layer or a silver layer.

The electron injection layer is a layer which injects electrons from an electrode, and is preferably a compound which has a capability of transporting electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film. Specific examples thereof include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

The organic light emitting device according to the present disclosure may be a front side emission type, a backside emission type, or a double-sided emission type according to the used material.

The preparation of the compound represented by the Chemical Formula 1 or Chemical Formula 2 and the organic light emitting device including the same will be described in detail in the following examples. However, these examples are presented for illustrative purposes only, and are not intended to limit the scope of the present disclosure.

### PREPARATION EXAMPLES

### Preparation Example 1: Preparation of Compound AA

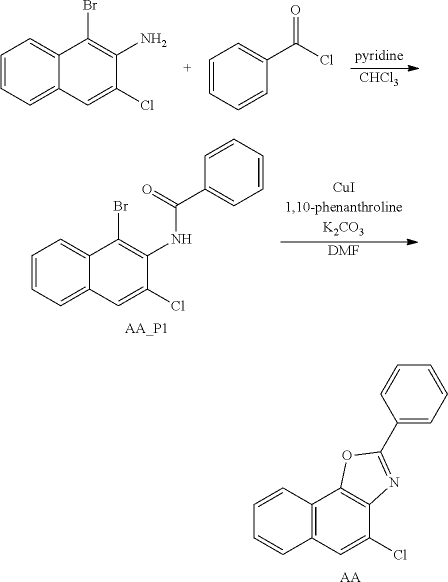

1-bromo-3-chloronaphthalen-2-amine (15 g, 58.5 mmol) and benzoyl chloride (9.9 g, 70.2 mmol) were added to chloroform (300 ml) under a nitrogen atmosphere, and stirred. After that, pyridine (6.9 g, 87.7 mmol) was added dropwise. After 9 hours of reaction at room temperature, ethanol (600 ml) was added for solidification. The resulting solid was filtered, dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 17 g of Compound AA_P1 (Yield 81%, MS: [M+H]⁺=360).

Compound AA_P1 (15 g, 41.6 mmol) and potassium carbonate (17.2 g, 124.8 mmol) were added to DMF (150 ml) under a nitrogen atmosphere, and the mixture was stirred and refluxed. After sufficient stirring, copper iodide (0.1 g, 0.4 mmol) and 1,10-phenanthroline (0.1 g, 0.8 mmol) were added thereto. After 11 hours of reaction, cooling was performed to at room temperature and water (300 ml) was added for solidification. The resulting solid was filtered, dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 9.6 g of Compound AA (Yield 83%, MS: [M+H]⁺=280).

### Preparation Example 2: Preparation of Compound AB

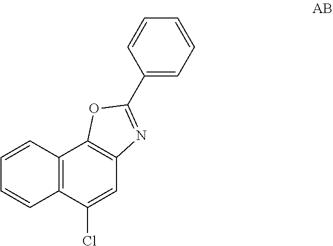 [SMILES: Clc1cc2nc(-c3ccccc3)oc2c2ccccc12]

Compound AB was prepared in the same manner as in Preparation Example 1, except that 1-bromo-4-chloronaphthalen-2-amine was used instead of 1-bromo-3-chloronaphthalen-2-amine.

### Preparation Example 3: Preparation of Compound AC

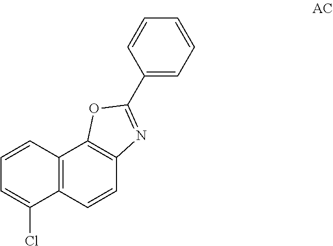 [SMILES: Clc1cccc2c1ccc1nc(-c3ccccc3)oc12]

Compound AC was prepared in the same manner as in Preparation Example 1, except that 1-bromo-5-chloronaphthalen-2-amine was used instead of 1-bromo-3-chloronaphthalen-2-amine.

### Preparation Example 4: Preparation of Compound AD

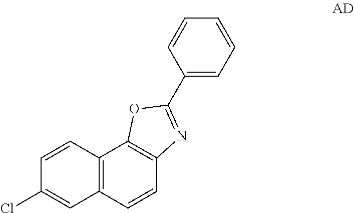 [SMILES: Clc1ccc2c(ccc3nc(-c4ccccc4)oc32)c1]

Compound AD was prepared in the same manner as in Preparation Example 1, except that 1-bromo-6-chloronaphthalen-2-amine was used instead of 1-bromo-3-chloronaphthalen-2-amine.

### Preparation Example 5: Preparation of Compound AE

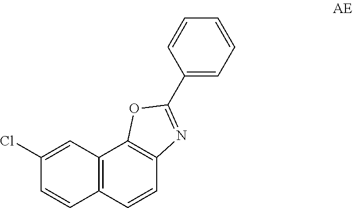 [SMILES: Clc1ccc2ccc3nc(-c4ccccc4)oc3c2c1]

Compound AE was prepared in the same manner as in Preparation Example 1, except that 1-bromo-7-chloronaphthalen-2-amine was used instead of 1-bromo-3-chloronaphthalen-2-amine.

### Preparation Example 6: Preparation of Compound AF

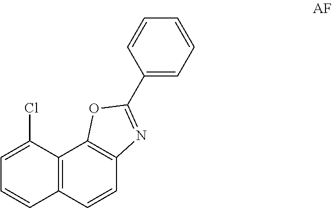 [SMILES: Clc1cccc2ccc3nc(-c4ccccc4)oc3c12]

Compound AF was prepared in the same manner as in Preparation Example 1, except that 1-bromo-8-chloronaphthalen-2-amine was used instead of 1-bromo-3-chloronaphthalen-2-amine.

### Preparation Example 7: Preparation of Compound AG

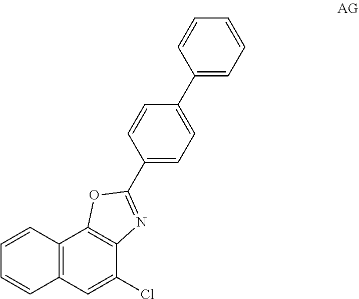 [SMILES: Clc1cc2ccccc2c2oc(-c3ccc(-c4ccccc4)cc3)nc12]

Compound AG was prepared in the same manner as in Preparation Example 1, except that [1,1′-biphenyl]-4-carbonyl chloride was used instead of benzoyl chloride.

### Preparation Example 8: Preparation of Compound AH

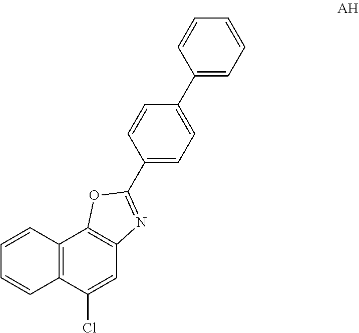 [SMILES: Clc1cc2nc(-c3ccc(-c4ccccc4)cc3)oc2c2ccccc12]

Compound AH was prepared in the same manner as in Preparation Example 1, except that [1,1′-biphenyl]-4-carbonyl chloride was used instead of benzoyl chloride, and 1-bromo-4-chloronaphthalen-2-amine was used instead of 1-bromo-3-chloronaphthalen-2-amine.

### Preparation Example 9: Preparation of Compound AI

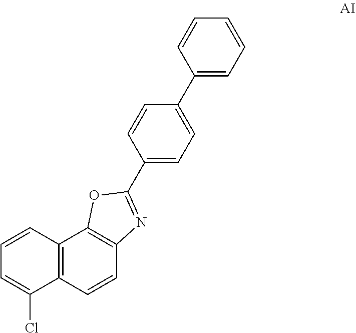 [SMILES: Clc1cccc2c1ccc1nc(-c3ccc(-c4ccccc4)cc3)oc12]

Compound AH was prepared in the same manner as in Preparation Example 1, except that [1,1′-biphenyl]-4-carbonyl chloride was used instead of benzoyl chloride, and 1-bromo-5-chloronaphthalen-2-amine was used instead of 1-bromo-3-chloronaphthalen-2-amine.

### Preparation Example 10: Preparation of Compound AJ

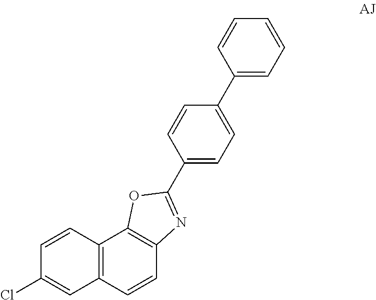 [SMILES: Clc1ccc2c(ccc3nc(-c4ccc(-c5ccccc5)cc4)oc32)c1]

Compound AJ was prepared in the same manner as in Preparation Example 1, except that [1,1′-biphenyl]-4-carbonyl chloride was used instead of benzoyl chloride, and 1-bromo-6-chloronaphthalen-2-amine was used instead of 1-bromo-3-chloronaphthalen-2-amine.

### Preparation Example 11: Preparation of Compound AK

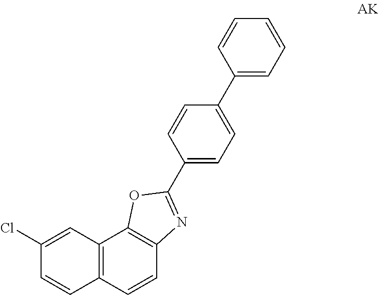 [SMILES: Clc1ccc2ccc3nc(-c4ccc(-c5ccccc5)cc4)oc3c2c1]

Compound AK was prepared in the same manner as in Preparation Example 1, except that [1,1′-biphenyl]-4-carbonyl chloride was used instead of benzoyl chloride, and 1-bromo-7-chloronaphthalen-2-amine was used instead of 1-bromo-3-chloronaphthalen-2-amine.

### Preparation Example 12: Preparation of Compound AL

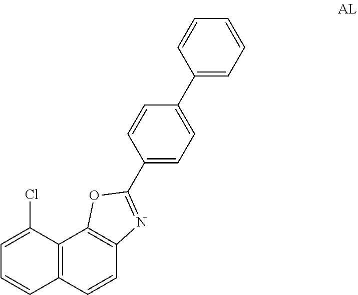 [SMILES: Clc1cccc2ccc3nc(-c4ccc(-c5ccccc5)cc4)oc3c12]

Compound AL was prepared in the same manner as in Preparation Example 1, except that [1,1′-biphenyl]-4-carbonyl chloride was used instead of benzoyl chloride, and 1-bromo-8-chloronaphthalen-2-amine was used instead of 1-bromo-3-chloronaphthalen-2-amine.

### Preparation Example 13: Preparation of Compound AM

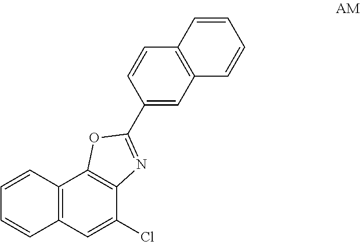 [SMILES: Clc1cc2ccccc2c2oc(-c3ccc4ccccc4c3)nc12]

Compound AM was prepared in the same manner as in Preparation Example 1, except that 2-naphthoyl chloride was used instead of benzoyl chloride.

### Preparation Example 14: Preparation of Compound AN

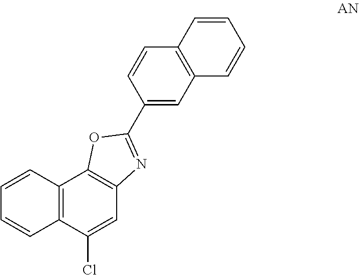 [SMILES: Clc1cc2nc(-c3ccc4ccccc4c3)oc2c2ccccc12]

Compound AN was prepared in the same manner as in Preparation Example 1, except that 2-naphthoyl chloride was used instead of benzoyl chloride, and 1-bromo-4-chloronaphthalen-2-amine was used instead of 1-bromo-3-chloronaphthalen-2-amine.

### Preparation Example 15: Preparation of Compound AO

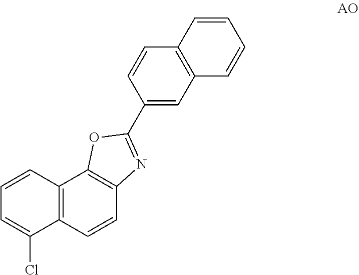 [SMILES: Clc1cccc2c1ccc1nc(-c3ccc4ccccc4c3)oc12]

Compound AO was prepared in the same manner as in Preparation Example 1, except that 2-naphthoyl chloride was used instead of benzoyl chloride, and 1-bromo-5-chloronaphthalen-2-amine was used instead of 1-bromo-3-chloronaphthalen-2-amine.

### Preparation Example 16: Preparation of Compound AP

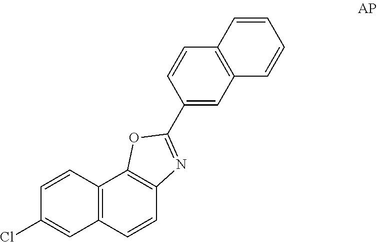 [SMILES: Clc1ccc2c(ccc3nc(-c4ccc5ccccc5c4)oc32)c1]

Compound AP was prepared in the same manner as in Preparation Example 1, except that 2-naphthoyl chloride was used instead of benzoyl chloride, and 1-bromo-6-chloronaphthalen-2-amine was used instead of 1-bromo-3-chloronaphthalen-2-amine.

### Preparation Example 17: Preparation of Compound AQ

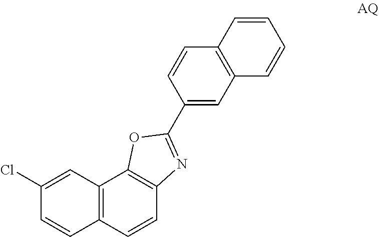 [SMILES: Clc1ccc2ccc3nc(-c4ccc5ccccc5c4)oc3c2c1]

Compound AQ was prepared in the same manner as in Preparation Example 1, except that 2-naphthoyl chloride was used instead of benzoyl chloride, and 1-bromo-7-chloronaphthalen-2-amine was used instead of 1-bromo-3-chloronaphthalen-2-amine.

### Preparation Example 18: Preparation of Compound AR

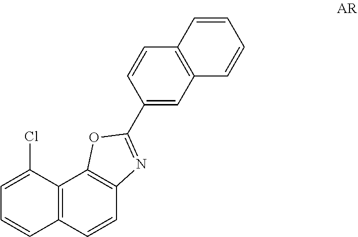 [SMILES: Clc1cccc2ccc3nc(-c4ccc5ccccc5c4)oc3c12]

Compound AR was prepared in the same manner as in Preparation Example 1, except that 2-naphthoyl chloride was used instead of benzoyl chloride, and 1-bromo-8-chloronaphthalen-2-amine was used instead of 1-bromo-3-chloronaphthalen-2-amine.

### Preparation Example 19: Preparation of Compound BA

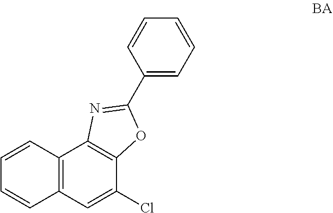 [SMILES: Clc1cc2ccccc2c2nc(-c3ccccc3)oc12]

Compound BA was prepared in the same manner as in Preparation Example 1, except that 2-bromo-3-chloronaphthalen-1-amine was used instead of 1-bromo-3-chloronaphthalen-2-amine.

### Preparation Example 20: Preparation of Compound BB

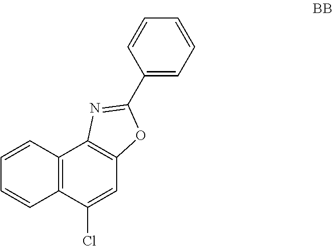 [SMILES: Clc1cc2oc(-c3ccccc3)nc2c2ccccc12]

Compound BB was prepared in the same manner as in Preparation Example 1, except that 2-bromo-4-chloronaphthalen-1-amine was used instead of 1-bromo-3-chloronaphthalen-2-amine.

### Preparation Example 21: Preparation of Compound BC

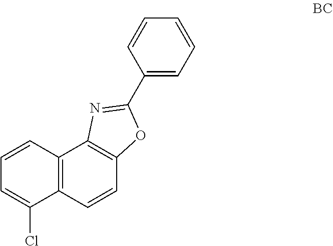 [SMILES: Clc1cccc2c1ccc1oc(-c3ccccc3)nc12]

Compound BC was prepared in the same manner as in Preparation Example 1, except that 2-bromo-5-chloronaphthalen-1-amine was used instead of 1-bromo-3-chloronaphthalen-2-amine.

### Preparation Example 22: Preparation of Compound BD

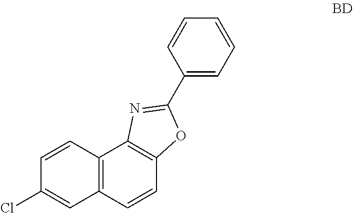 [SMILES: Clc1ccc2c(ccc3oc(-c4ccccc4)nc32)c1]

Compound BD was prepared in the same manner as in Preparation Example 1, except that 2-bromo-6-chloronaphthalen-1-amine was used instead of 1-bromo-3-chloronaphthalen-2-amine.

### Preparation Example 23: Preparation of Compound BE

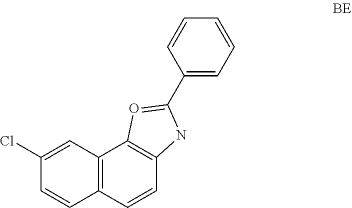

Compound BE was prepared in the same manner as in Preparation Example 1, except that 2-bromo-7-chloronaphthalen-1-amine was used instead of 1-bromo-3-chloronaphthalen-2-amine.

### Preparation Example 24: Preparation of Compound BF

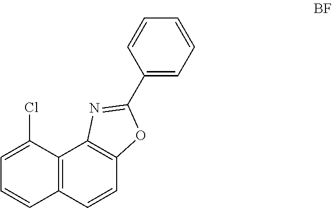 [SMILES: Clc1cccc2ccc3oc(-c4ccccc4)nc3c12]

Compound BF was prepared in the same manner as in Preparation Example 1, except that 2-bromo-8-chloronaphthalen-1-amine was used instead of 1-bromo-3-chloronaphthalen-2-amine.

### Preparation Example 25: Preparation of Compound BG

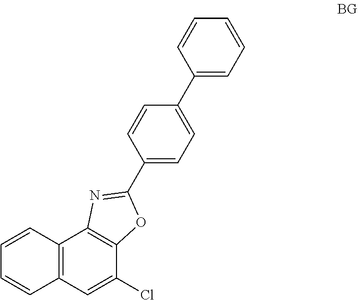 [SMILES: Clc1cc2ccccc2c2nc(-c3ccc(-c4ccccc4)cc3)oc12]

Compound BG was prepared in the same manner as in Preparation Example 1, except that [1,1′-biphenyl]-4-carbonyl chloride was used instead of benzoyl chloride, and 2-bromo-3-chloronaphthalen-1-amine was used instead of 1-bromo-3-chloronaphthalen-2-amine.

### Preparation Example 26: Preparation of Compound BH

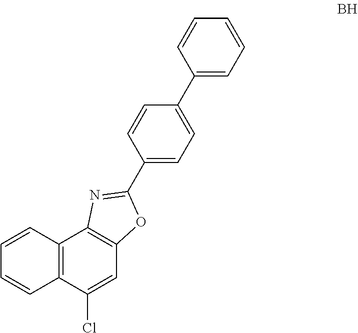 [SMILES: Clc1cc2oc(-c3ccc(-c4ccccc4)cc3)nc2c2ccccc12]

Compound BH was prepared in the same manner as in Preparation Example 1, except that [1,1′-biphenyl]-4-carbonyl chloride was used instead of benzoyl chloride, and 2-bromo-4-chloronaphthalen-1-amine was used instead of 1-bromo-3-chloronaphthalen-2-amine.

### Preparation Example 27: Preparation of Compound BI

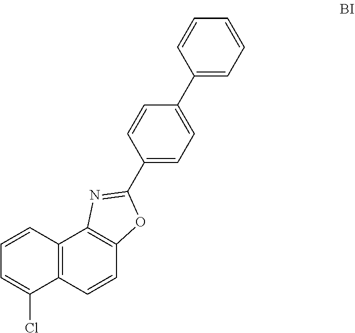 [SMILES: Clc1cccc2c1ccc1oc(-c3ccc(-c4ccccc4)cc3)nc12]

Compound BI was prepared in the same manner as in Preparation Example 1, except that [1,1′-biphenyl]-4-carbonyl chloride was used instead of benzoyl chloride, and 2-bromo-3-chloronaphthalen-1-amine was used instead of 1-bromo-3-chloronaphthalen-2-amine.

### Preparation Example 28: Preparation of Compound BJ

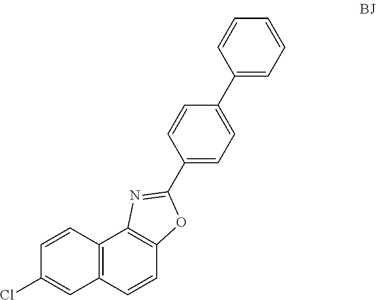 [SMILES: Clc1ccc2c(ccc3oc(-c4ccc(-c5ccccc5)cc4)nc32)c1]

Compound BJ was prepared in the same manner as in Preparation Example 1, except that [1,1′-biphenyl]-4-carbonyl chloride was used instead of benzoyl chloride, and 2-bromo-6-chloronaphthalen-1-amine was used instead of 1-bromo-3-chloronaphthalen-2-amine.

### Preparation Example 29: Preparation of Compound BK

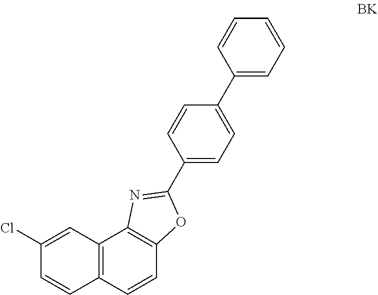 [SMILES: Clc1ccc2ccc3oc(-c4ccc(-c5ccccc5)cc4)nc3c2c1]

Compound BK was prepared in the same manner as in Preparation Example 1, except that [1,1′-biphenyl]-4-carbonyl chloride was used instead of benzoyl chloride, and 2-bromo-7-chloronaphthalen-1-amine was used instead of 1-bromo-3-chloronaphthalen-2-amine.

### Preparation Example 30: Preparation of Compound BL

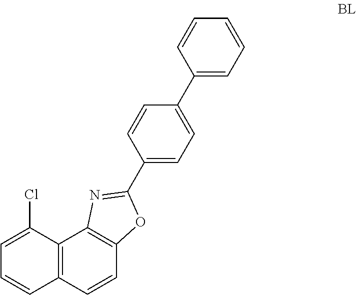 [SMILES: Clc1cccc2ccc3oc(-c4ccc(-c5ccccc5)cc4)nc3c12]

Compound BL was prepared in the same manner as in Preparation Example 1, except that [1,1′-biphenyl]-4-carbonyl chloride was used instead of benzoyl chloride, and 2-bromo-8-chloronaphthalen-1-amine was used instead of 1-bromo-3-chloronaphthalen-2-amine.

### Preparation Example 31: Preparation of Compound BM

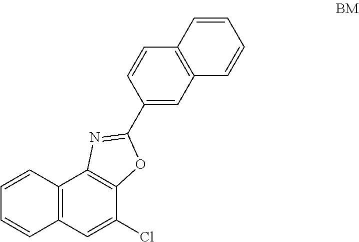 [SMILES: Clc1cc2ccccc2c2nc(-c3ccc4ccccc4c3)oc12]

Compound BM was prepared in the same manner as in Preparation Example 1, except that 2-naphthoyl chloride was used instead of benzoyl chloride, and 2-bromo-3-chloronaphthalen-1-amine was used instead of 1-bromo-3-chloronaphthalen-2-amine.

### Preparation Example 32: Preparation of Compound BN

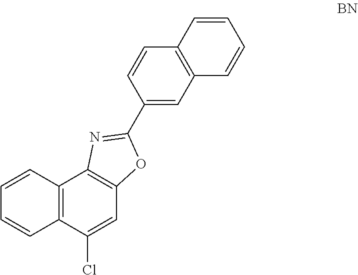 [SMILES: Clc1cc2oc(-c3ccc4ccccc4c3)nc2c2ccccc12]

Compound BN was prepared in the same manner as in Preparation Example 1, except that 2-naphthoyl chloride was used instead of benzoyl chloride, and 2-bromo-4-chloronaphthalen-1-amine was used instead of 1-bromo-3-chloronaphthalen-2-amine.

### Preparation Example 33: Preparation of Compound BO

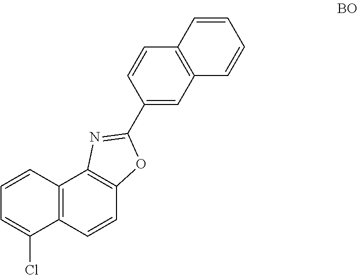 [SMILES: Clc1cccc2c1ccc1oc(-c3ccc4ccccc4c3)nc12]

Compound BO was prepared in the same manner as in Preparation Example 1, except that 2-naphthoyl chloride was used instead of benzoyl chloride, and 2-bromo-5-chloronaphthalen-1-amine was used instead of 1-bromo-3-chloronaphthalen-2-amine.

### Preparation Example 34: Preparation of Compound BP

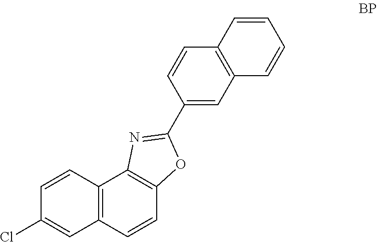 [SMILES: Clc1ccc2c(ccc3oc(-c4ccc5ccccc5c4)nc32)c1]

Compound BP was prepared in the same manner as in Preparation Example 1, except that 2-naphthoyl chloride was used instead of benzoyl chloride, and 2-bromo-6-chloronaphthalen-1-amine was used instead of 1-bromo-3-chloronaphthalen-2-amine.

### Preparation Example 35: Preparation of Compound BQ

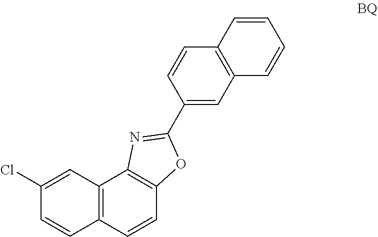 [SMILES: Clc1ccc2ccc3oc(-c4ccc5ccccc5c4)nc3c2c1]

Compound BQ was prepared in the same manner as in Preparation Example 1, except that 2-naphthoyl chloride was used instead of benzoyl chloride, and 2-bromo-7-chloronaphthalen-1-amine was used instead of 1-bromo-3-chloronaphthalen-2-amine.

### Preparation Example 36: Preparation of Compound BR

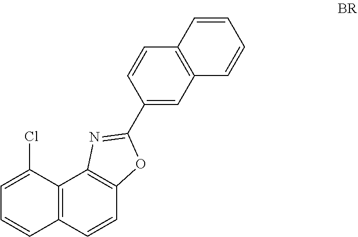 [SMILES: Clc1cccc2ccc3oc(-c4ccc5ccccc5c4)nc3c12]

Compound BR was prepared in the same manner as in Preparation Example 1, except that 2-naphthoyl chloride was used instead of benzoyl chloride, and 2-bromo-8-chloronaphthalen-1-amine was used instead of 1-bromo-3-chloronaphthalen-2-amine.

### EXAMPLES

### Example 1: Preparation of Compound 1

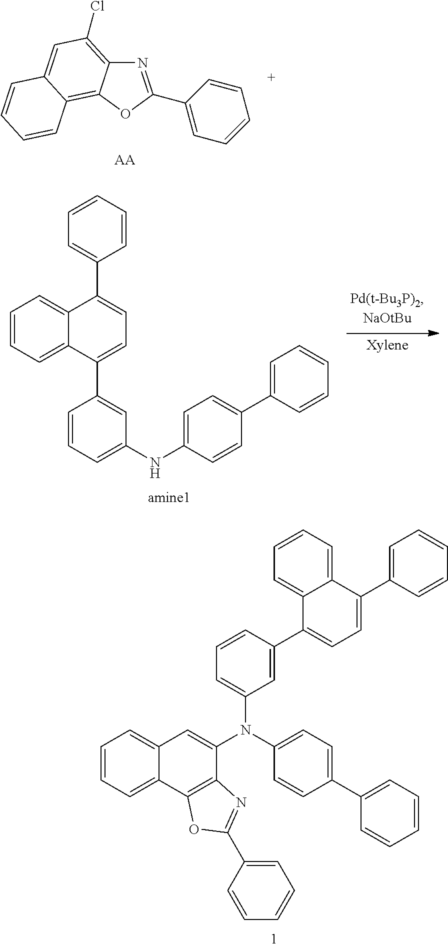

Compound AA (10 g, 35.8 mmol), Compound amine1 (16 g, 35.8 mmol), and sodium tert-butoxide (11.4 g, 53.6 mmol) were added to xylene (200 ml) under a nitrogen atmosphere, and the mixture was stirred and refluxed. Thereafter, bis(tri-tert-butylphosphine)palladium(0) (0.2 g, 0.4 mmol) was added thereto. Upon completion of the reaction after 2 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 16.8 g of Compound 1 (Yield 68%, MS: [M+H]⁺=691).

### Example 2: Preparation of Compound 2

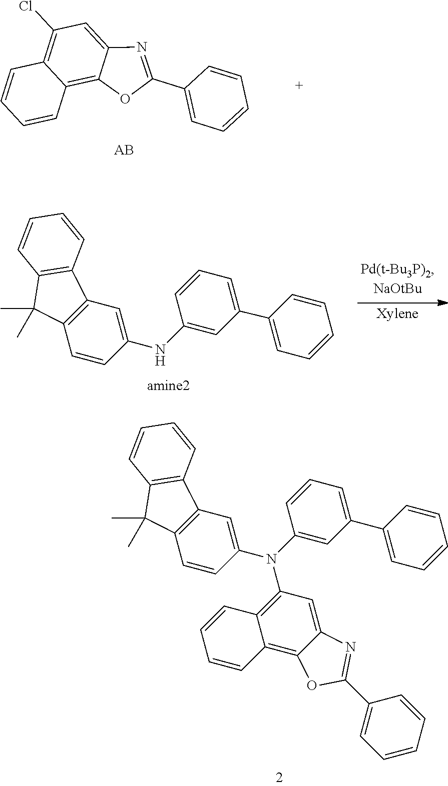 [SMILES: Clc1cc2nc(-c3ccccc3)oc2c2ccccc12 ; CC1(C)c2ccccc2-c2cc(Nc3cccc(-c4ccccc4)c3)ccc21 ; CC1(C)c2ccccc2-c2cc(N(c3cccc(-c4ccccc4)c3)c3cc4nc(-c5ccccc5)oc4c4ccccc34)ccc21]

Compound AB (10 g, 35.8 mmol), Compound amine2 (12.9 g, 35.8 mmol), and sodium tert-butoxide (11.4 g, 53.6 mmol) were added to xylene (200 ml) under a nitrogen atmosphere, and the mixture was stirred and refluxed. Thereafter, bis(tri-tert-butylphosphine)palladium(0) (0.2 g, 0.4 mmol) was added thereto. Upon completion of the reaction after 2 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 13.2 g of Compound 2 (Yield 61%, MS: [M+H]⁺=605).

### Example 3: Preparation of Compound 3

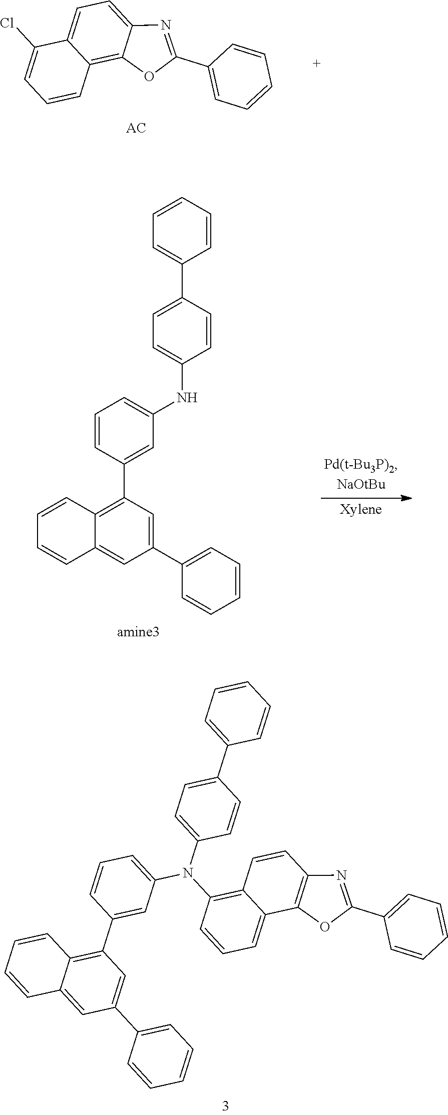 [SMILES: Clc1cccc2c1ccc1nc(-c3ccccc3)oc12 ; c1ccc(-c2ccc(Nc3cccc(-c4cc(-c5ccccc5)cc5ccccc45)c3)cc2)cc1 ; c1ccc(-c2ccc(N(c3cccc(-c4cc(-c5ccccc5)cc5ccccc45)c3)c3cccc4c3ccc3nc(-c5ccccc5)oc34)cc2)cc1]

Compound AC (10 g, 35.8 mmol), Compound amine3 (16 g, 35.8 mmol), and sodium tert-butoxide (11.4 g, 53.6 mmol) were added to xylene (200 ml) under a nitrogen atmosphere, and the mixture was stirred and refluxed. Thereafter, bis(tri-tert-butylphosphine)palladium(0) (0.2 g, 0.4 mmol) was added thereto. Upon completion of the reaction after 2 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 17.3 g of Compound 3 (Yield 70%, MS: [M+H]⁺=691).

### Example 4: Preparation of Compound 4

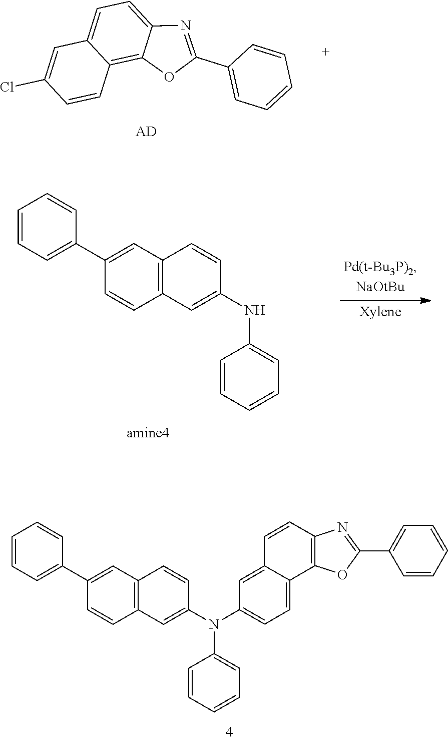

Compound AD (10 g, 35.8 mmol), Compound amine4 (10.6 g, 35.8 mmol), and sodium tert-butoxide (11.4 g, 53.6 mmol) were added to xylene (200 ml) under a nitrogen atmosphere, and the mixture was stirred and refluxed. Thereafter, bis(tri-tert-butylphosphine)palladium(0) (0.2 g, 0.4 mmol) was added thereto. Upon completion of the reaction after 3 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 12.7 g of Compound 4 (Yield 66%, MS: [M+H]⁺=539).

### Example 5: Preparation of Compound 5

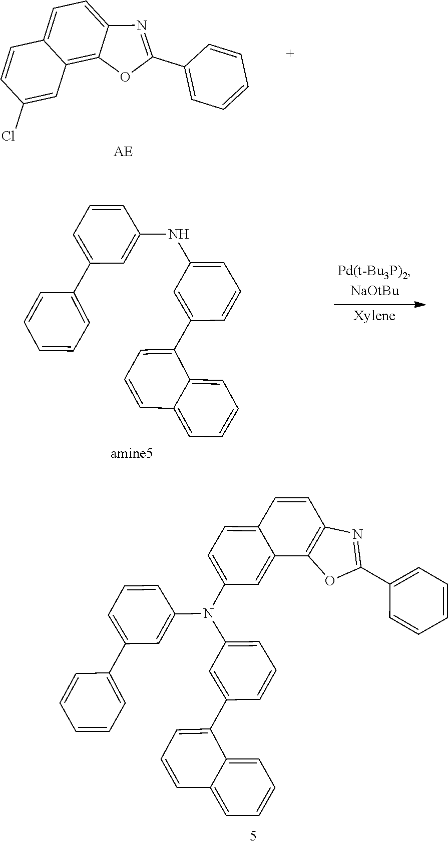 [SMILES: Clc1ccc2ccc3nc(-c4ccccc4)oc3c2c1 ; "AE" ; c1ccc(-c2cccc(Nc3cccc(-c4cccc5ccccc45)c3)c2)cc1 ; c1ccc(-c2cccc(N(c3cccc(-c4cccc5ccccc45)c3)c3ccc4ccc5nc(-c6ccccc6)oc5c4c3)c2)cc1]

Compound AE (10 g, 35.8 mmol), Compound amine5 (13.3 g, 35.8 mmol), and sodium tert-butoxide (11.4 g, 53.6 mmol) were added to xylene (200 ml) under a nitrogen atmosphere, and the mixture was stirred and refluxed. Thereafter, bis(tri-tert-butylphosphine)palladium(0) (0.2 g, 0.4 mmol) was added thereto. Upon completion of the reaction after 2 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 13.4 g of Compound 5 (Yield 61%, MS: [M+H]⁺=615).

### Example 6: Preparation of Compound 6

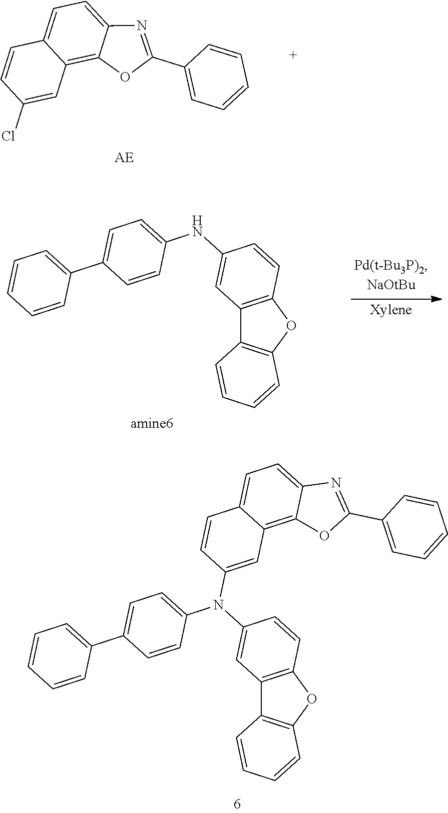 [SMILES: Clc1ccc2ccc3nc(-c4ccccc4)oc3c2c1 ; "AE" ; c1ccc(-c2ccc(Nc3ccc4oc5ccccc5c4c3)cc2)cc1 ; c1ccc(-c2ccc(N(c3ccc4oc5ccccc5c4c3)c3ccc4ccc5nc(-c6ccccc6)oc5c4c3)cc2)cc1]

Compound AE (10 g, 35.8 mmol), Compound amine6 (12 g, 35.8 mmol), and sodium tert-butoxide (11.4 g, 53.6 mmol) were added to xylene (200 ml) under a nitrogen atmosphere, and the mixture was stirred and refluxed. Thereafter, bis(tri-tert-butylphosphine)palladium(0) (0.2 g, 0.4 mmol) was added thereto. Upon completion of the reaction after 3 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 13.4 g of Compound 6 (Yield 65%, MS: [M+H]⁺=579).

### Example 7: Preparation of Compound 7

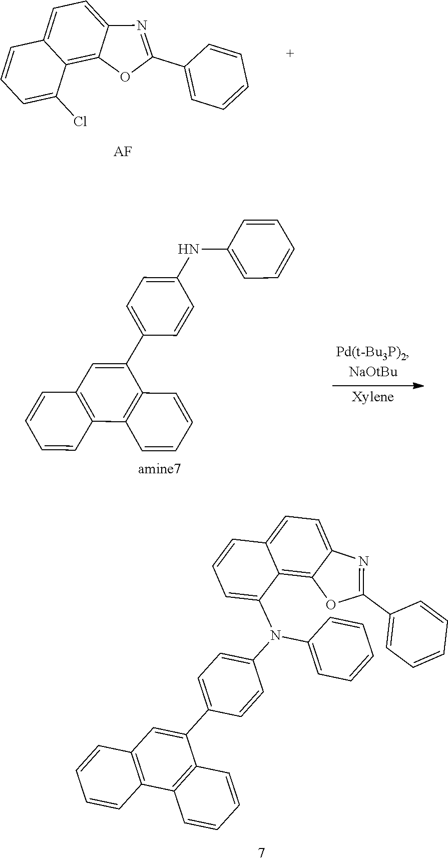

Compound AF (10 g, 35.8 mmol), Compound amine7 (12.3 g, 35.8 mmol), and sodium tert-butoxide (11.4 g, 53.6 mmol) were added to xylene (200 ml) under a nitrogen atmosphere, and the mixture was stirred and refluxed. Thereafter, bis(tri-tert-butylphosphine)palladium(0) (0.2 g, 0.4 mmol) was added thereto. Upon completion of the reaction after 2 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 13.5 g of Compound 7 (Yield 64%, MS: [M+H]⁺=589).

### Example 8: Preparation of Compound 8

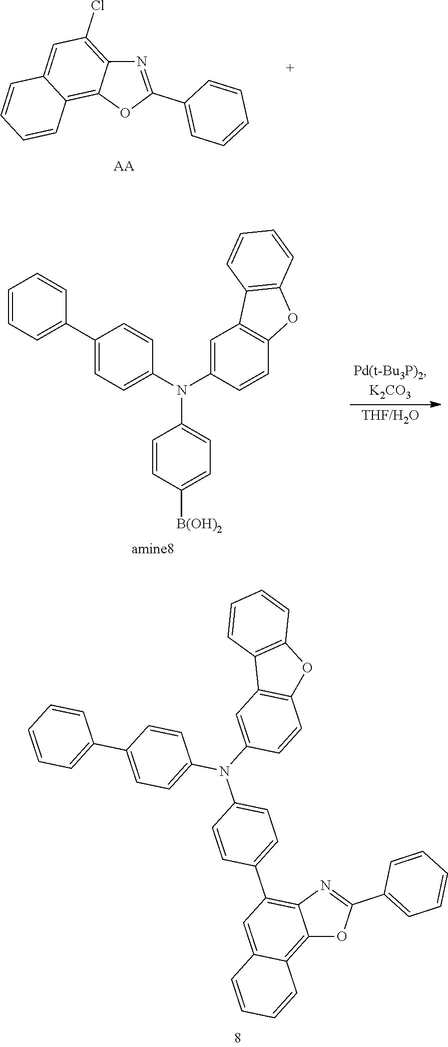 [SMILES: Clc1cc2ccccc2c2oc(-c3ccccc3)nc12 ; "AA" ; OB(O)c1ccc(N(c2ccc(-c3ccccc3)cc2)c2ccc3oc4ccccc4c3c2)cc1 ; c1ccc(-c2ccc(N(c3ccc(-c4cc5ccccc5c5oc(-c6ccccc6)nc45)cc3)c3ccc4oc5ccccc5c4c3)cc2)cc1]

Compound AA (15 g, 53.6 mmol), and Compound amine8 (25.6 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 23.9 g of Compound 8 (Yield 68%, MS: [M+H]⁺=655).

### Example 9: Preparation of Compound 9

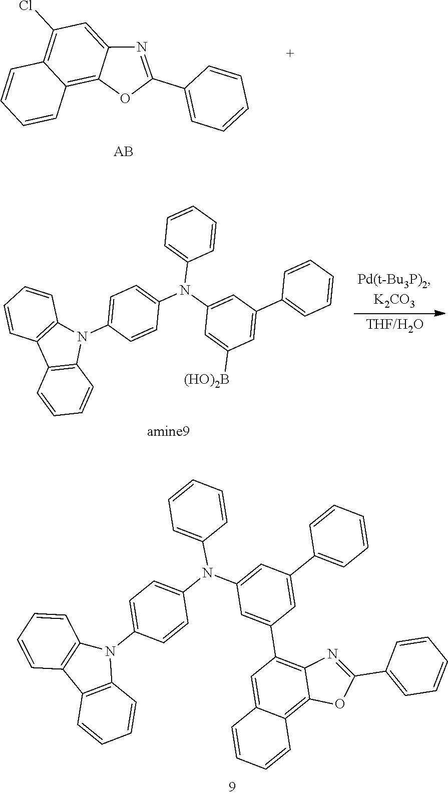 [SMILES: Clc1cc2nc(-c3ccccc3)oc2c2ccccc12 ; "AB" ; OB(O)c1cc(-c2ccccc2)cc(N(c2ccccc2)c2ccc(-n3c4ccccc4c4ccccc43)cc2)c1 ; c1ccc(-c2cc(-c3cc4ccccc4c4oc(-c5ccccc5)nc34)cc(N(c3ccccc3)c3ccc(-n4c5ccccc5c5ccccc54)cc3)c2)cc1]

Compound AB (15 g, 53.6 mmol) and Compound amine9 (29.9 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 23.9 g of Compound 9 (Yield 61%, MS: [M+H]⁺=730).

### Example 10: Preparation of Compound 10

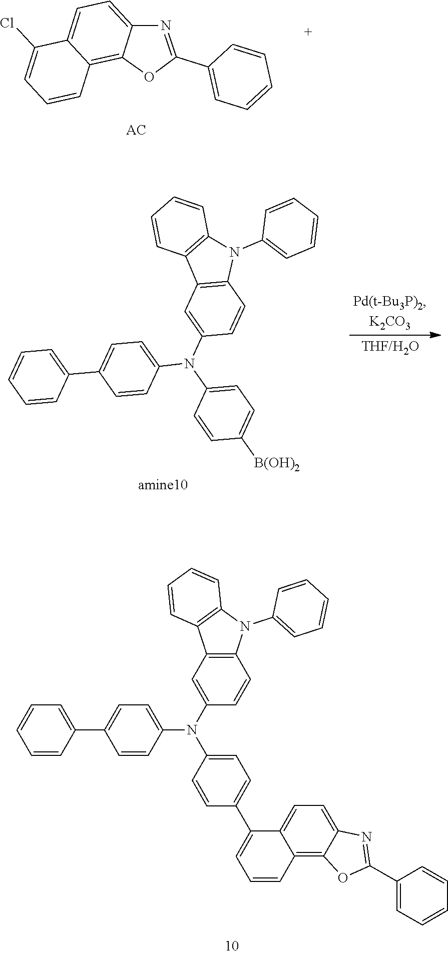 [SMILES: Clc1cccc2c1ccc1nc(-c3ccccc3)oc12 ; "AC" ; OB(O)c1ccc(N(c2ccc(-c3ccccc3)cc2)c2ccc3c(c2)c2ccccc2n3-c2ccccc2)cc1 ; c1ccc(-c2ccc(N(c3ccc(-c4cccc5c4ccc4nc(-c6ccccc6)oc45)cc3)c3ccc4c(c3)c3ccccc3n4-c3ccccc3)cc2)cc1]

Compound AC (15 g, 53.6 mmol) and Compound amine10 (29.9 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 24.2 g of Compound 10 (Yield 62%, MS: [M+H]⁺=730).

### Example 11: Preparation of Compound 11

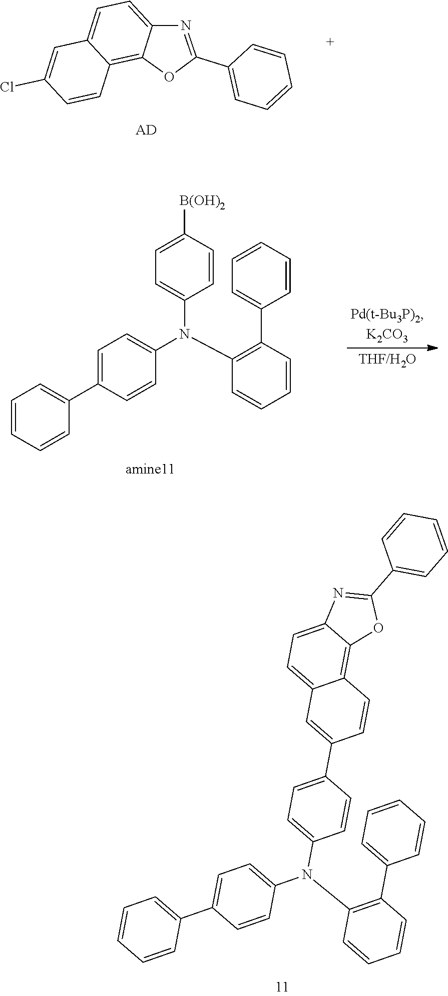

Compound AD (15 g, 53.6 mmol) and Compound amine11 (24.9 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20.9 g of Compound 11 (Yield 61%, MS: [M+H]⁺=641).

### Example 12: Preparation of Compound 12

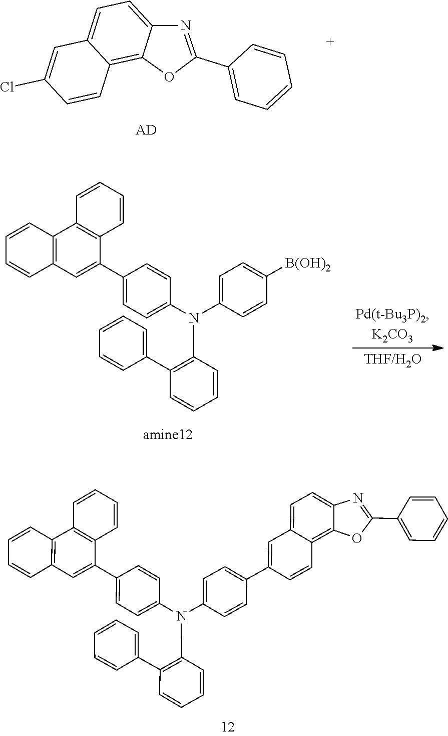 [SMILES: Clc1ccc2c(ccc3nc(-c4ccccc4)oc32)c1 ; "AD" ; OB(O)c1ccc(N(c2ccc(-c3cc4ccccc4c4ccccc34)cc2)c2ccccc2-c2ccccc2)cc1 ; c1ccc(-c2nc3ccc4cc(-c5ccc(N(c6ccc(-c7cc8ccccc8c8ccccc78)cc6)c6ccccc6-c6ccccc6)cc5)ccc4c3o2)cc1]

Compound AD (15 g, 53.6 mmol) and Compound amine12 (30.5 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 25.8 g of Compound 12 (Yield 65%, MS: [M+H]⁺=741).

### Example 13: Preparation of Compound 13

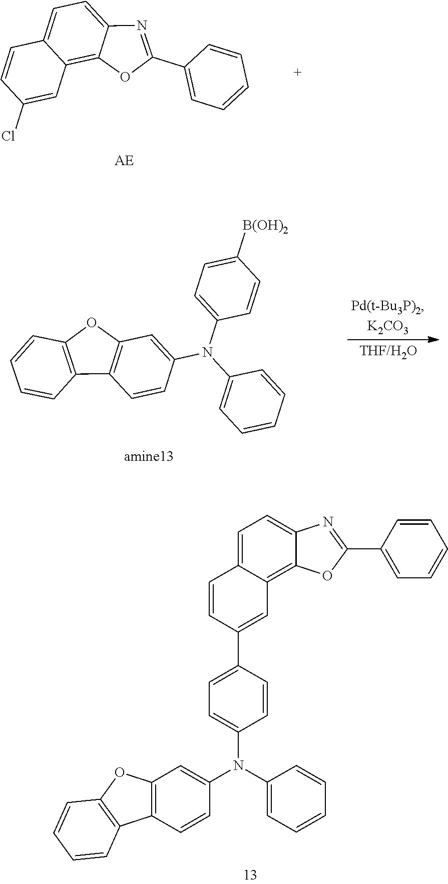 [SMILES: Clc1ccc2ccc3nc(-c4ccccc4)oc3c2c1 ; "AE" ; OB(O)c1ccc(N(c2ccccc2)c2ccc3c(c2)oc2ccccc23)cc1 ; c1ccc(-c2nc3ccc4ccc(-c5ccc(N(c6ccccc6)c6ccc7c(c6)oc6ccccc67)cc5)cc4c3o2)cc1]

Compound AE (15 g, 53.6 mmol) and Compound amine13 (21.4 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 18.6 g of Compound 13 (Yield 60%, MS: [M+H]⁺=579).

### Example 14: Preparation of Compound 14

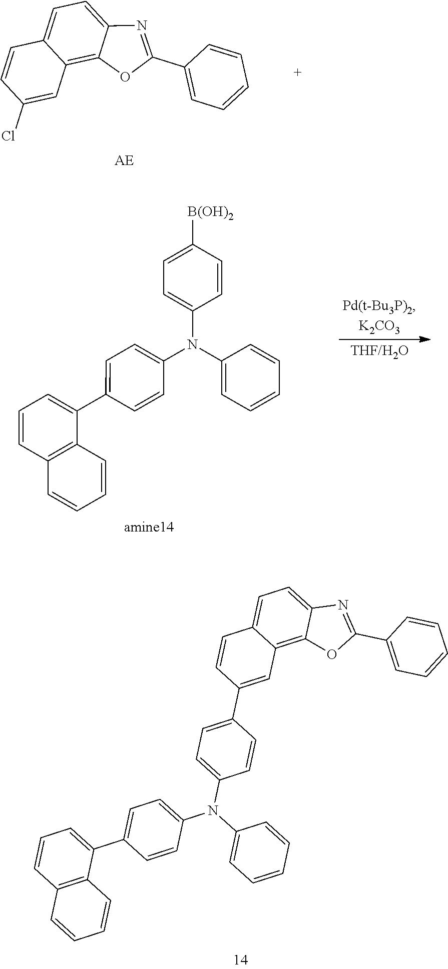 [SMILES: Clc1ccc2ccc3nc(-c4ccccc4)oc3c2c1 ; "AE" ; OB(O)c1ccc(N(c2ccccc2)c2ccc(-c3cccc4ccccc34)cc2)cc1 ; c1ccc(-c2nc3ccc4ccc(-c5ccc(N(c6ccccc6)c6ccc(-c7cccc8ccccc78)cc6)cc5)cc4c3o2)cc1]

Compound AE (15 g, 53.6 mmol) and Compound amine14 (23.4 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 22.1 g of Compound 14 (Yield 67%, MS: [M+H]⁺=615).

### Example 15: Preparation of Compound 15

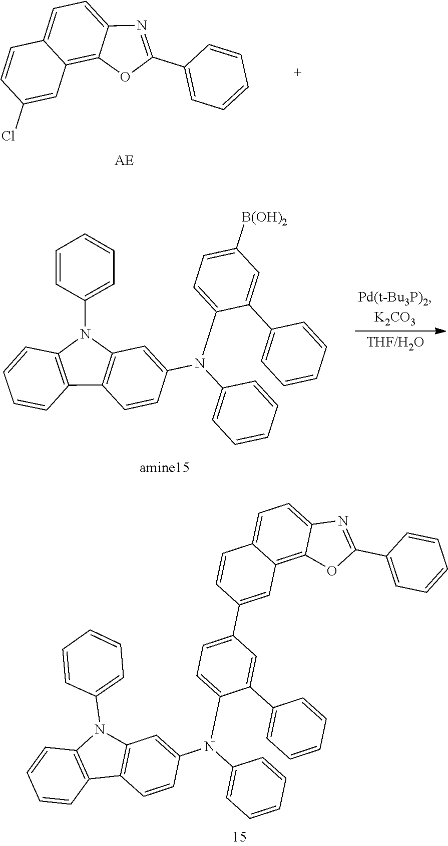 [SMILES: Clc1ccc2ccc3nc(-c4ccccc4)oc3c2c1 ; "AE" ; OB(O)c1ccc(N(c2ccccc2)c2ccc3c4ccccc4n(-c4ccccc4)c3c2)c(-c2ccccc2)c1 ; c1ccc(-c2nc3ccc4ccc(-c5ccc(N(c6ccccc6)c6ccc7c8ccccc8n(-c8ccccc8)c7c6)c(-c6ccccc6)c5)cc4c3o2)cc1]

Compound AE (15 g, 53.6 mmol) and Compound amine15 (29.9 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 24.2 g of Compound 15 (Yield 62%, MS: [M+H]⁺=730).

### Example 16: Preparation of Compound 16

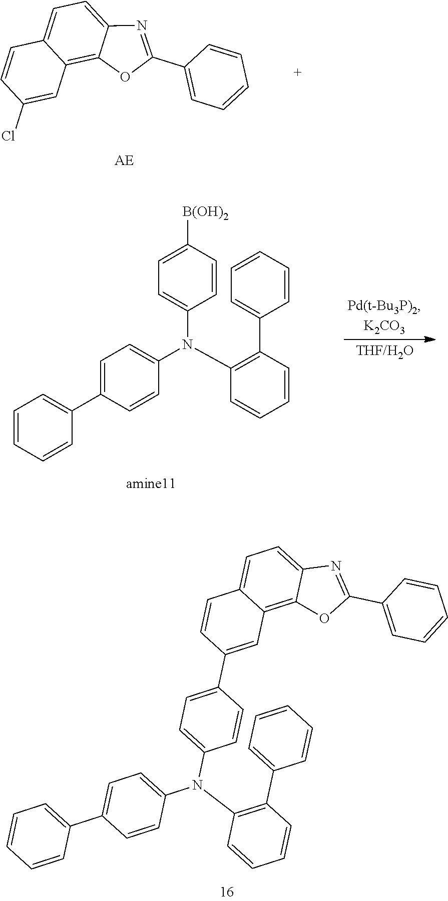 [SMILES: Clc1ccc2ccc3nc(-c4ccccc4)oc3c2c1 ; "AE" ; OB(O)c1ccc(N(c2ccc(-c3ccccc3)cc2)c2ccccc2-c2ccccc2)cc1 ; c1ccc(-c2ccc(N(c3ccc(-c4ccc5ccc6nc(-c7ccccc7)oc6c5c4)cc3)c3ccccc3-c3ccccc3)cc2)cc1]

Compound AE (15 g, 53.6 mmol) and Compound amine11 (24.9 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 23 g of Compound 16 (Yield 67%, MS: [M+H]⁺=641).

### Example 17: Preparation of Compound 17

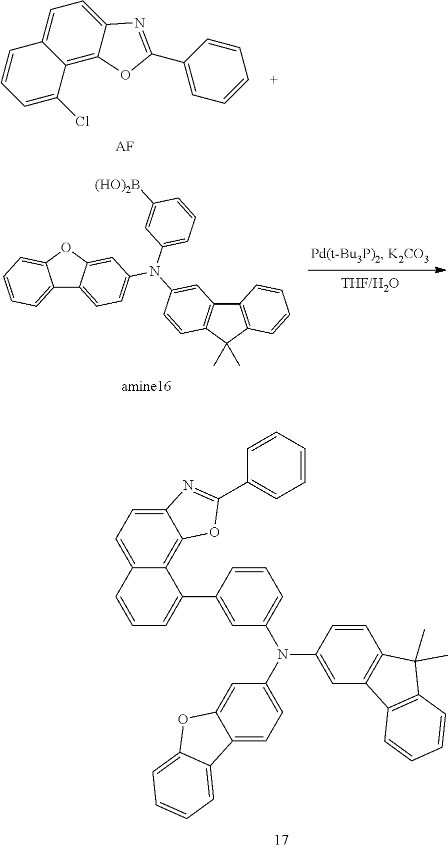

Compound AF (15 g, 53.6 mmol) and Compound amine16 (27.9 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 23.5 g of Compound 17 (Yield 63%, MS: [M+H]⁺=695).

### Example 18: Preparation of Compound 18

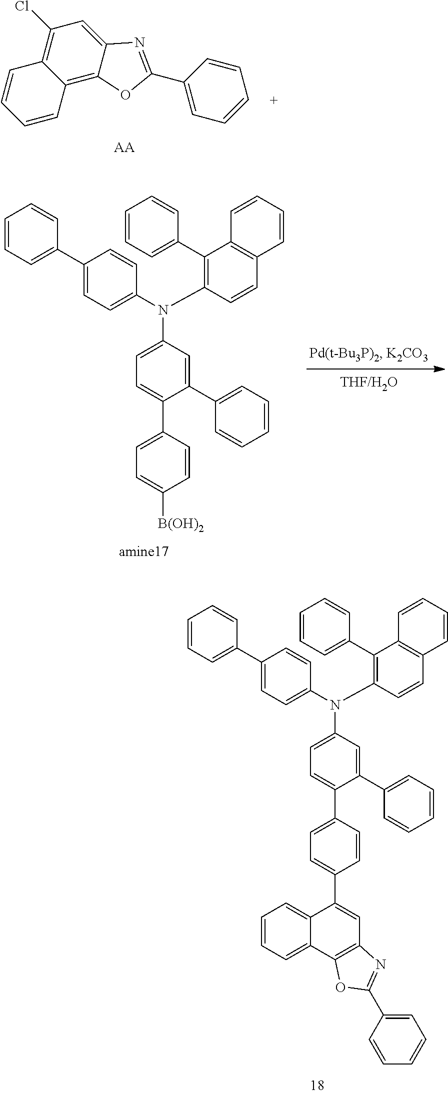 [SMILES: Clc1cc2nc(-c3ccccc3)oc2c2ccccc12 ; "AA" ; OB(O)c1ccc(-c2ccc(N(c3ccc(-c4ccccc4)cc3)c3ccc4ccccc4c3-c3ccccc3)cc2-c2ccccc2)cc1 ; c1ccc(-c2ccc(N(c3ccc(-c4ccc(-c5cc6nc(-c7ccccc7)oc6c6ccccc56)cc4)c(-c4ccccc4)c3)c3ccc4ccccc4c3-c3ccccc3)cc2)cc1]

Compound AA (15 g, 53.6 mmol) and Compound amine17 (36.2 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 28.5 g of Compound 18 (Yield 63%, MS: [M+H]⁺=843).

### Example 19: Preparation of Compound 19

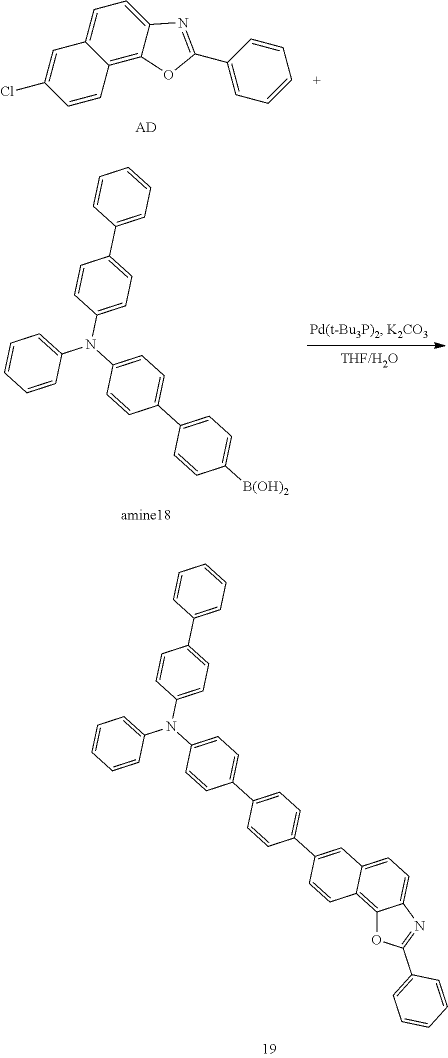

Compound AD (15 g, 53.6 mmol) and Compound amine18 (24.9 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 23.3 g of Compound 19 (Yield 68%, MS: [M+H]⁺=641).

### Example 20: Preparation of Compound 20

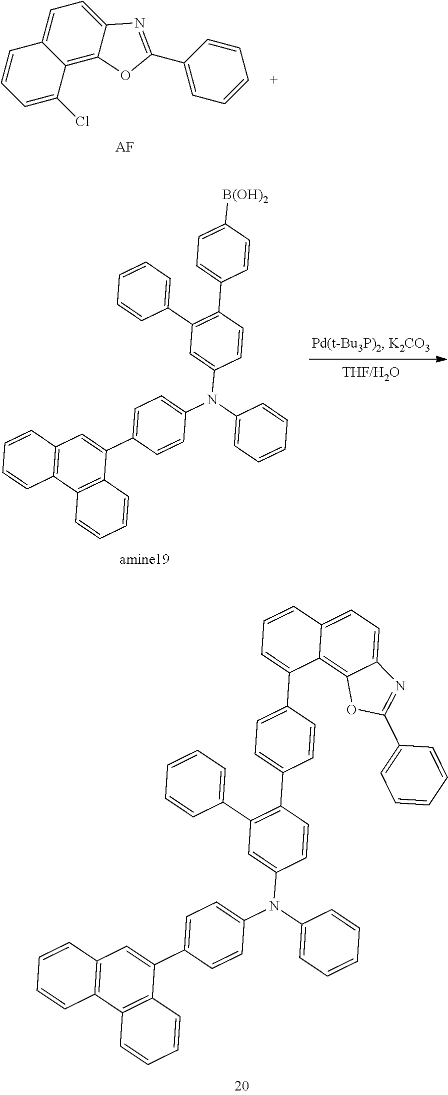

Compound AF (15 g, 53.6 mmol) and Compound amine19 (34.8 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 27.6 g of Compound 20 (Yield 63%, MS: [M+H]⁺=817).

### Example 21: Preparation of Compound 21

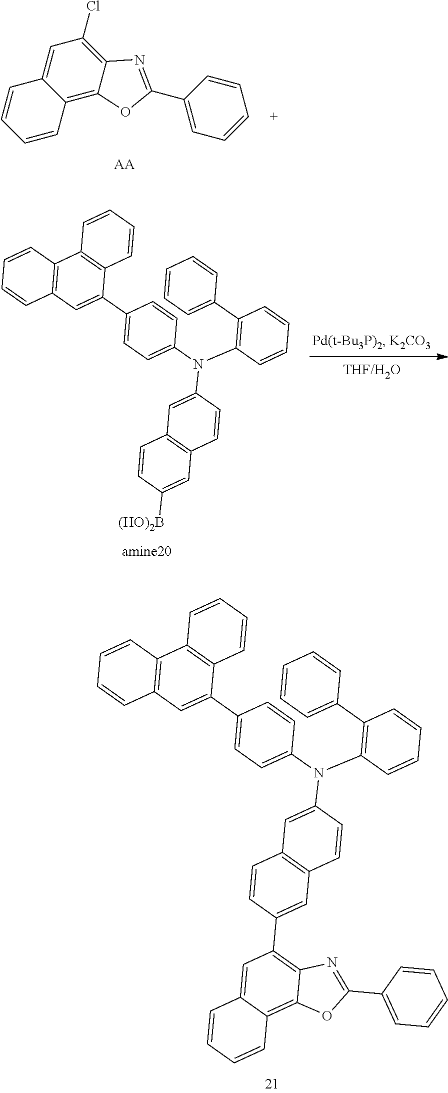 [SMILES: Clc1cc2ccccc2c2oc(-c3ccccc3)nc12 ; "AA" ; OB(O)c1ccc2cc(N(c3ccc(-c4cc5ccccc5c5ccccc45)cc3)c3ccccc3-c3ccccc3)ccc2c1 ; c1ccc(-c2nc3c(-c4ccc5cc(N(c6ccc(-c7cc8ccccc8c8ccccc78)cc6)c6ccccc6-c6ccccc6)ccc5c4)cc4ccccc4c3o2)cc1]

Compound AA (15 g, 53.6 mmol) and Compound amine20 (33.3 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 27.5 g of Compound 21 (Yield 65%, MS: [M+H]⁺=791).

### Example 22: Preparation of Compound 22

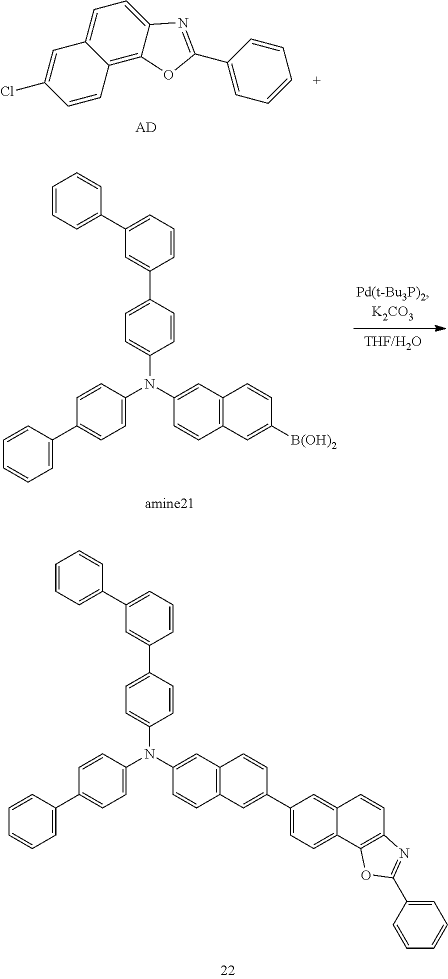

Compound AD (15 g, 53.6 mmol) and Compound amine21 (32 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 27.9 g of Compound 22 (Yield 68%, MS: [M+H]⁺=767).

### Example 23: Preparation of Compound 23

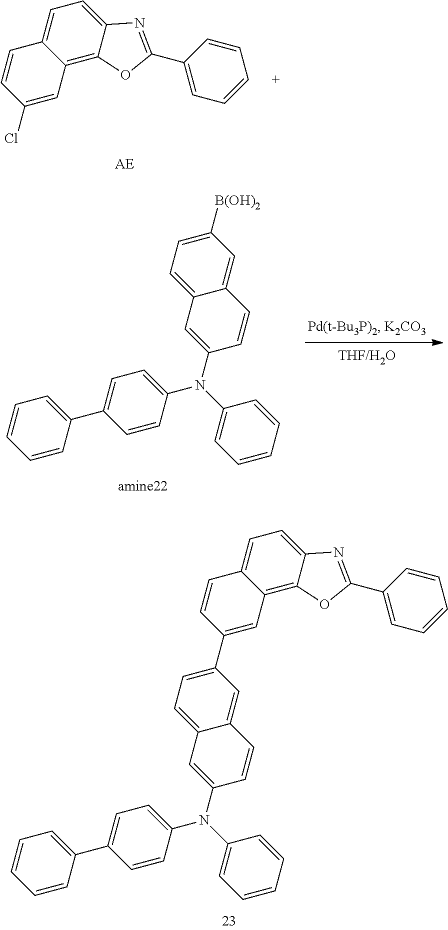 [SMILES: Clc1ccc2ccc3nc(-c4ccccc4)oc3c2c1 ; "AE" ; OB(O)c1ccc2cc(N(c3ccccc3)c3ccc(-c4ccccc4)cc3)ccc2c1 ; c1ccc(-c2ccc(N(c3ccccc3)c3ccc4cc(-c5ccc6ccc7nc(-c8ccccc8)oc7c6c5)ccc4c3)cc2)cc1]

Compound AE (15 g, 53.6 mmol) and Compound amine22 (23.4 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 22.4 g of Compound 23 (Yield 68%, MS: [M+H]⁺=615).

### Example 24: Preparation of Compound 24

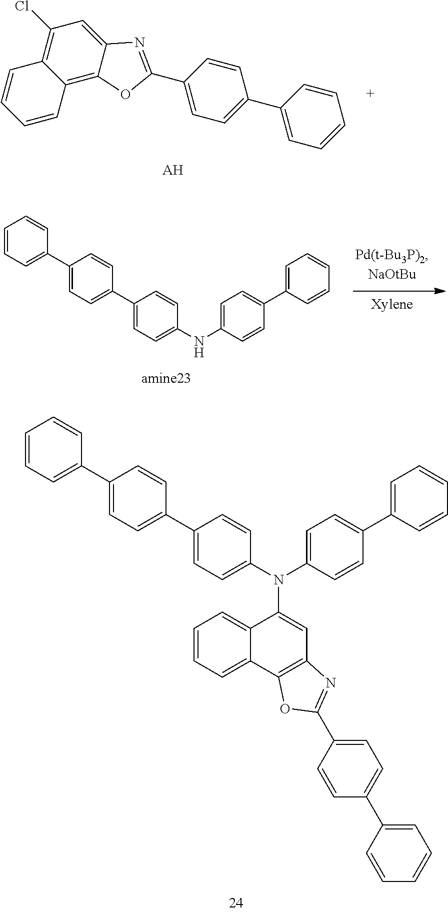 [SMILES: Clc1cc2nc(-c3ccc(-c4ccccc4)cc3)oc2c2ccccc12 ; "AH" ; c1ccc(-c2ccc(Nc3ccc(-c4ccc(-c5ccccc5)cc4)cc3)cc2)cc1 ; c1ccc(-c2ccc(-c3ccc(N(c4ccc(-c5ccccc5)cc4)c4cc5nc(-c6ccc(-c7ccccc7)cc6)oc5c5ccccc45)cc3)cc2)cc1]

Compound AH (10 g, 28.1 mmol), Compound amine23 (11.2 g, 28.1 mmol), and sodium tert-butoxide (8.9 g, 42.2 mmol) were added to xylene (200 ml) under a nitrogen atmosphere, and the mixture was stirred and refluxed. Thereafter, bis(tri-tert-butylphosphine)palladium(0) (0.1 g, 0.2 mmol) was added thereto. Upon completion of the reaction after 3 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 14.1 g of Compound 24 (Yield 70%, MS: [M+H]⁺=717).

### Example 25, Preparation of Compound 25

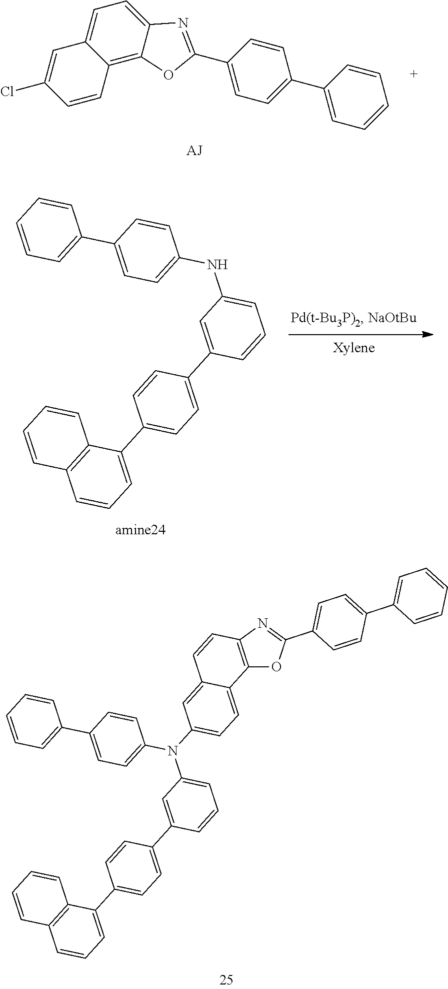 [SMILES: Clc1ccc2c(ccc3nc(-c4ccc(-c5ccccc5)cc4)oc32)c1 ; "AJ" ; c1ccc(-c2ccc(Nc3cccc(-c4ccc(-c5cccc6ccccc56)cc4)c3)cc2)cc1 ; c1ccc(-c2ccc(-c3nc4ccc5cc(N(c6ccc(-c7ccccc7)cc6)c6cccc(-c7ccc(-c8cccc9ccccc89)cc7)c6)ccc5c4o3)cc2)cc1]

Compound AJ (10 g, 28.1 mmol), Compound amine24 (12.6 g, 28.1 mmol), and sodium tert-butoxide (8.9 g, 42.2 mmol) were added to xylene (200 ml) under a nitrogen atmosphere, and the mixture was stirred and refluxed. Thereafter, bis(tri-tert-butylphosphine)palladium(0) (0.1 g, 0.2 mmol) was added thereto. Upon completion of the reaction after 2 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 13.1 g of Compound 25 (Yield 61%, MS: [M+H]⁺=767).

### Example 26: Preparation of Compound 26

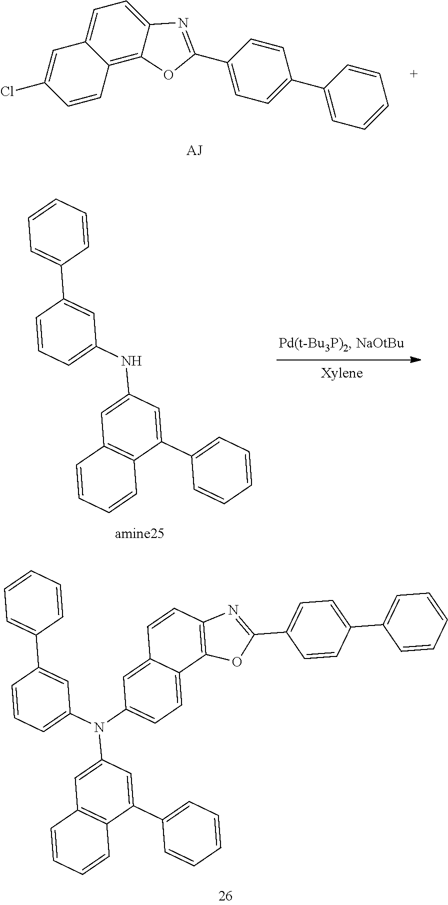 [SMILES: Clc1ccc2c(ccc3nc(-c4ccc(-c5ccccc5)cc4)oc32)c1 ; "AJ" ; c1ccc(-c2cccc(Nc3cc(-c4ccccc4)c4ccccc4c3)c2)cc1 ; c1ccc(-c2ccc(-c3nc4ccc5cc(N(c6cccc(-c7ccccc7)c6)c6cc(-c7ccccc7)c7ccccc7c6)ccc5c4o3)cc2)cc1]

Compound AJ (10 g, 28.1 mmol), Compound amine25 (10.4 g, 28.1 mmol), and sodium tert-butoxide (8.9 g, 42.2 mmol) were added to xylene (200 ml) under a nitrogen atmosphere, and the mixture was stirred and refluxed. Thereafter, bis(tri-tert-butylphosphine)palladium(0) (0.1 g, 0.2 mmol) was added thereto. Upon completion of the reaction after 2 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 11.8 g of Compound 26 (Yield 61%, MS: [M+H]⁺=691).

### Example 27: Preparation of Compound 27

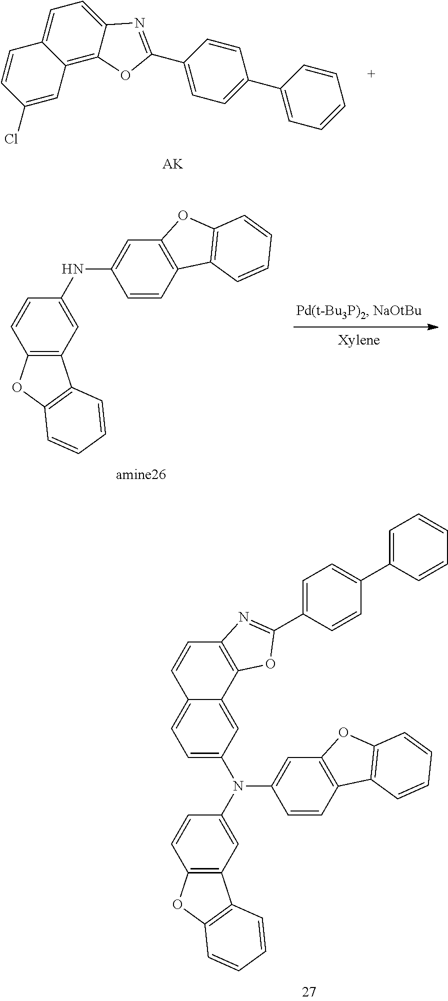 [SMILES: Clc1ccc2ccc3nc(-c4ccc(-c5ccccc5)cc4)oc3c2c1 ; "AK" ; c1ccc2c(c1)oc1cc(Nc3ccc4oc5ccccc5c4c3)ccc12 ; c1ccc(-c2ccc(-c3nc4ccc5ccc(N(c6ccc7c(c6)oc6ccccc67)c6ccc7oc8ccccc8c7c6)cc5c4o3)cc2)cc1]

Compound AK (10 g, 28.1 mmol), Compound amine26 (9.8 g, 28.1 mmol), and sodium tert-butoxide (8.9 g, 42.2 mmol) were added to xylene (200 ml) under a nitrogen atmosphere, and the mixture was stirred and refluxed. Thereafter, bis(tri-tert-butylphosphine)palladium(0) (0.1 g, 0.2 mmol) was added thereto. Upon completion of the reaction after 2 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 11.6 g of Compound 27 (Yield 62%, MS: [M+H]⁺=669).

### Example 28: Preparation of Compound 28

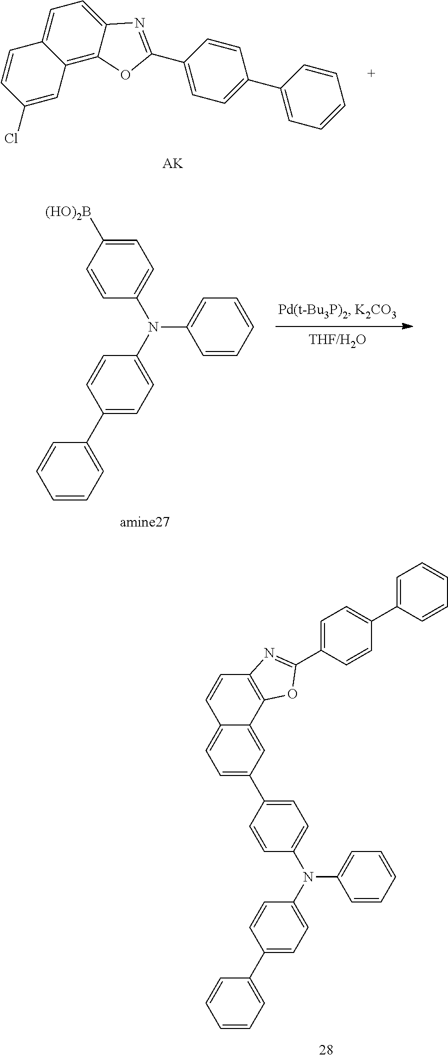 [SMILES: Clc1ccc2ccc3nc(-c4ccc(-c5ccccc5)cc4)oc3c2c1 ; "AK" ; OB(O)c1ccc(N(c2ccccc2)c2ccc(-c3ccccc3)cc2)cc1 ; c1ccc(-c2ccc(-c3nc4ccc5ccc(-c6ccc(N(c7ccccc7)c7ccc(-c8ccccc8)cc7)cc6)cc5c4o3)cc2)cc1]

Compound AK (15 g, 42.2 mmol) and Compound amine27 (16.2 g, 44.3 mmol) were added to THE (300 ml) under a nitrogen atmosphere, and the mixture was stirred and refluxed. Then, potassium carbonate (17.5 g, 126.5 mmol) was dissolved in water (52 ml), and then added thereto. Thereafter, it was stirred sufficiently, followed by adding bis(tri-tert-butylphosphine)palladium(0) (0.5 g, 1.0 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 16.2 g of Compound 28 (Yield 60%, MS: [M+H]⁺=641).

### Example 29: Preparation of Compound 29

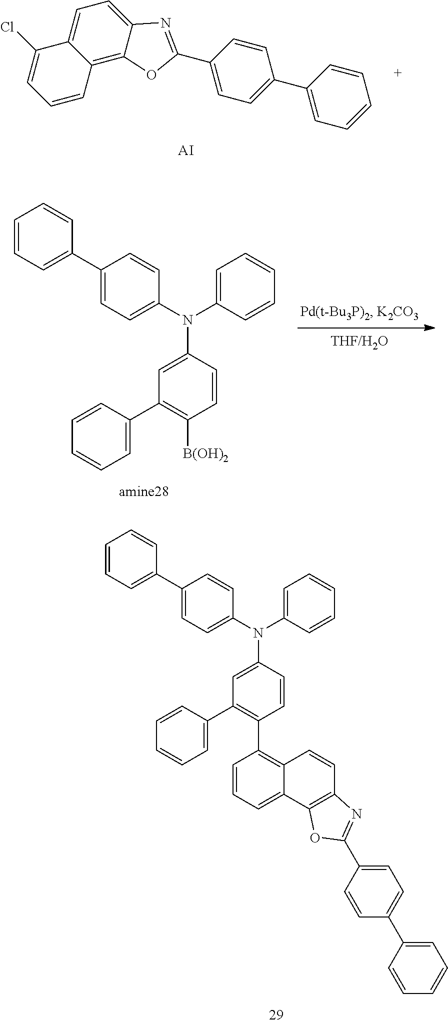 [SMILES: Clc1cccc2c1ccc1nc(-c3ccc(-c4ccccc4)cc3)oc12 ; "AI" ; OB(O)c1ccc(N(c2ccccc2)c2ccc(-c3ccccc3)cc2)cc1-c1ccccc1 ; c1ccc(-c2ccc(-c3nc4ccc5c(-c6ccc(N(c7ccccc7)c7ccc(-c8ccccc8)cc7)cc6-c6ccccc6)cccc5c4o3)cc2)cc1]

Compound AI (15 g, 42.2 mmol) and Compound amine28 (19.5 g, 44.3 mmol) were added to THE (300 ml) under a nitrogen atmosphere, and the mixture was stirred and refluxed. Then, potassium carbonate (17.5 g, 126.5 mmol) was dissolved in water (52 ml), and then added thereto. Thereafter, it was stirred sufficiently, followed by adding bis(tri-tert-butylphosphine)palladium(0) (0.5 g, 1.0 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 18.1 g of Compound 29 (Yield 60%, MS: [M+H]⁺=717).

### Example 30: Preparation of Compound 30

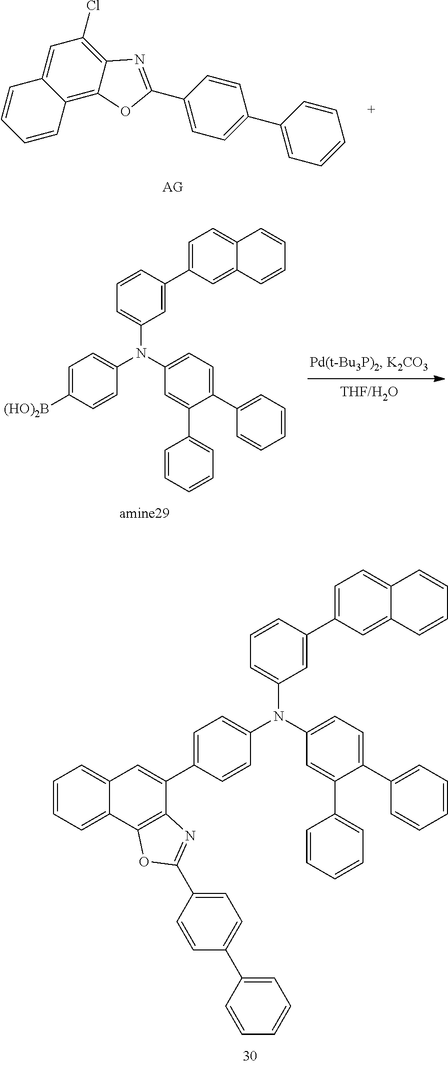 [SMILES: Clc1cc2ccccc2c2oc(-c3ccc(-c4ccccc4)cc3)nc12 ; "AG" ; OB(O)c1ccc(N(c2cccc(-c3ccc4ccccc4c3)c2)c2ccc(-c3ccccc3)c(-c3ccccc3)c2)cc1 ; c1ccc(-c2ccc(-c3nc4c(-c5ccc(N(c6cccc(-c7ccc8ccccc8c7)c6)c6ccc(-c7ccccc7)c(-c7ccccc7)c6)cc5)cc5ccccc5c4o3)cc2)cc1]

Compound AG (15 g, 42.2 mmol) and Compound amine29 (25.1 g, 44.3 mmol) were added to THE (300 ml) under a nitrogen atmosphere, and the mixture was stirred and refluxed. Then, potassium carbonate (17.5 g, 126.5 mmol) was dissolved in water (52 ml), and then added thereto. Thereafter, it was stirred sufficiently, followed by adding bis(tri-tert-butylphosphine)palladium(0) (0.5 g, 1.0 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 22.7 g of Compound 30 (Yield 64%, MS: [M+H]⁺=843).

### Example 31: Preparation of Compound 31

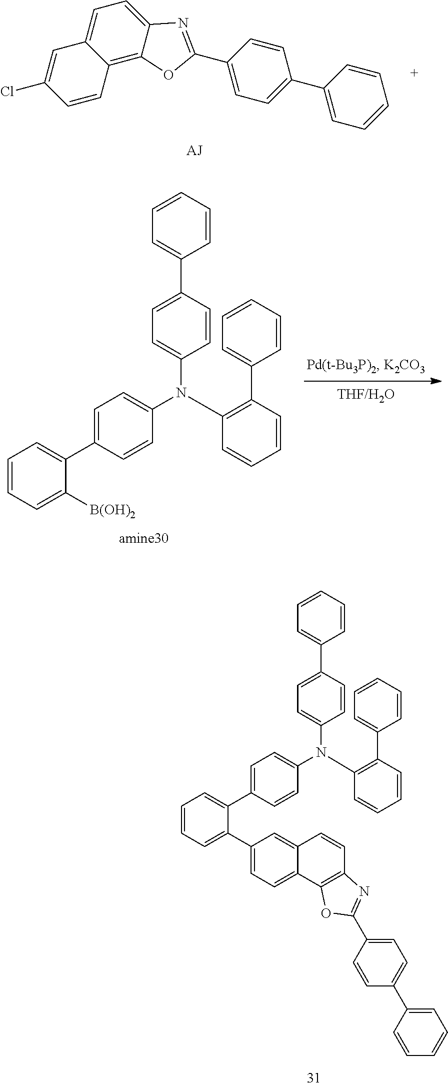

Compound AJ (15 g, 42.2 mmol) and Compound amine30 (22.9 g, 44.3 mmol) were added to THE (300 ml) under a nitrogen atmosphere, and the mixture was stirred and refluxed. Then, potassium carbonate (17.5 g, 126.5 mmol) was dissolved in water (52 ml), and then added thereto. Thereafter, it was stirred sufficiently, followed by adding bis(tri-tert-butylphosphine)palladium(0) (0.5 g, 1.0 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 22.4 g of Compound 31 (Yield 67%, MS: [M+H]⁺=793).

### Example 32: Preparation of Compound 32

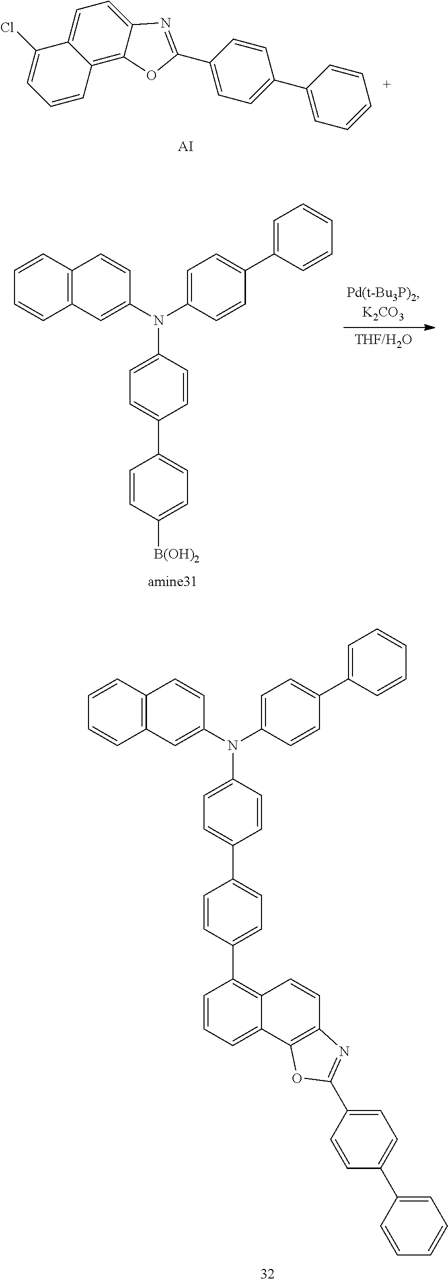 [SMILES: Clc1cccc2c1ccc1nc(-c3ccc(-c4ccccc4)cc3)oc12 ; "AI" ; OB(O)c1ccc(-c2ccc(N(c3ccc(-c4ccccc4)cc3)c3ccc4ccccc4c3)cc2)cc1 ; c1ccc(-c2ccc(-c3nc4ccc5c(-c6ccc(-c7ccc(N(c8ccc(-c9ccccc9)cc8)c8ccc9ccccc9c8)cc7)cc6)cccc5c4o3)cc2)cc1]

Compound AI (15 g, 42.2 mmol) and Compound amine31 (21.8 g, 44.3 mmol) were added to THE (300 ml) under a nitrogen atmosphere, and the mixture was stirred and refluxed. Then, potassium carbonate (17.5 g, 126.5 mmol) was dissolved in water (52 ml), and then added thereto. Thereafter, it was stirred sufficiently, followed by adding bis(tri-tert-butylphosphine)palladium(0) (0.5 g, 1.0 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 22.6 g of Compound 32 (Yield 70%, MS: [M+H]⁺=767).

### Example 33: Preparation of Compound 33

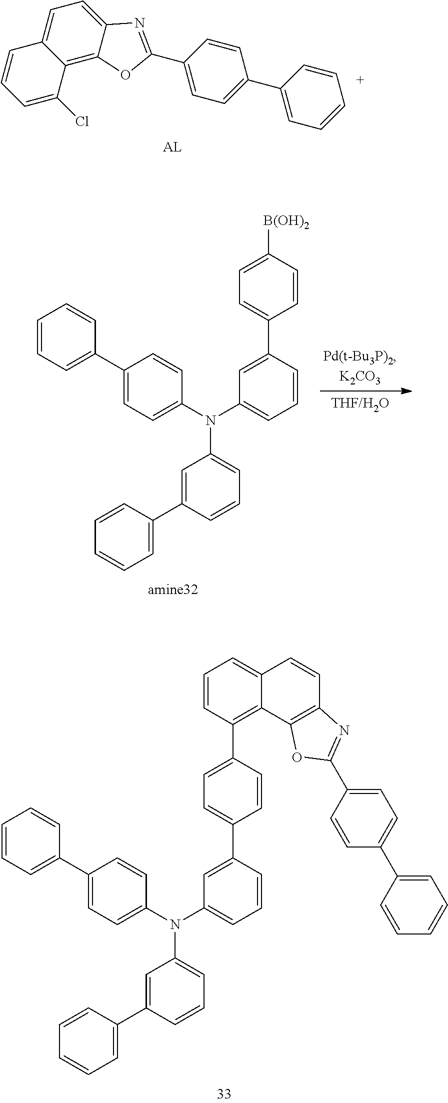 [SMILES: Clc1cccc2ccc3nc(-c4ccc(-c5ccccc5)cc4)oc3c12 ; "AL" ; OB(O)c1ccc(-c2cccc(N(c3ccc(-c4ccccc4)cc3)c3cccc(-c4ccccc4)c3)c2)cc1 ; c1ccc(-c2ccc(-c3nc4ccc5cccc(-c6ccc(-c7cccc(N(c8ccc(-c9ccccc9)cc8)c8cccc(-c9ccccc9)c8)c7)cc6)c5c4o3)cc2)cc1]

Compound AL (15 g, 42.2 mmol) and Compound amine32 (22.9 g, 44.3 mmol) were added to THE (300 ml) under a nitrogen atmosphere, and the mixture was stirred and refluxed. Then, potassium carbonate (17.5 g, 126.5 mmol) was dissolved in water (52 ml), and then added thereto. Thereafter, it was stirred sufficiently, followed by adding bis(tri-tert-butylphosphine)palladium(0) (0.5 g, 1.0 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 23.4 g of Compound 33 (Yield 70%, MS: [M+H]⁺=793).

### Example 34: Preparation of Compound 34

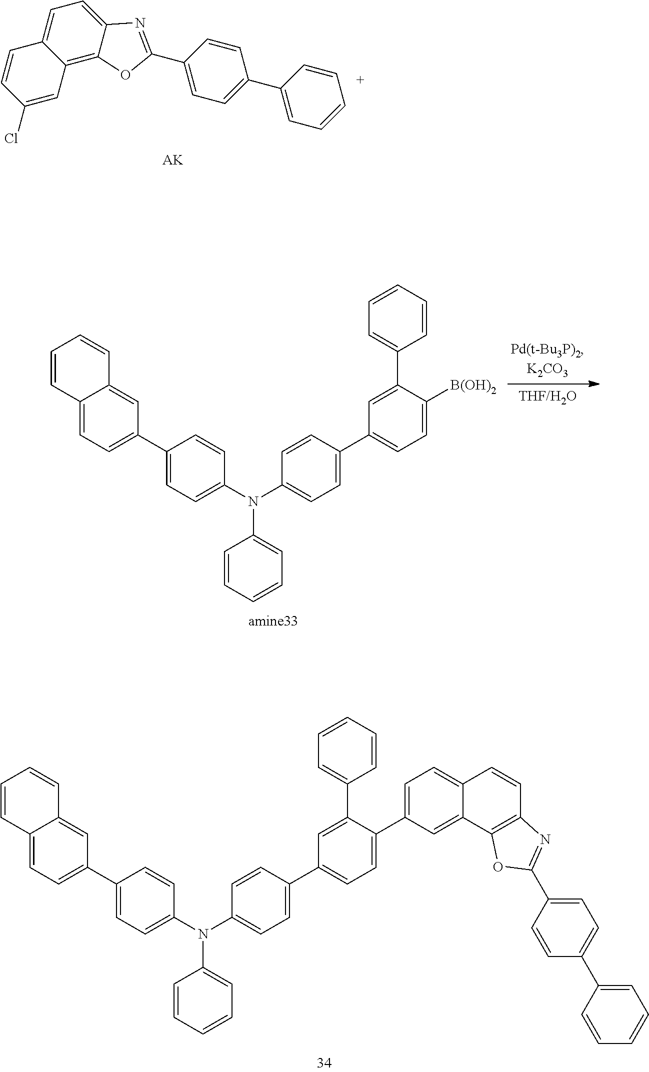 [SMILES: Clc1ccc2ccc3nc(-c4ccc(-c5ccccc5)cc4)oc3c2c1 ; "AK" ; OB(O)c1ccc(-c2ccc(N(c3ccccc3)c3ccc(-c4ccc5ccccc5c4)cc3)cc2)cc1-c1ccccc1 ; c1ccc(-c2ccc(-c3nc4ccc5ccc(-c6ccc(-c7ccc(N(c8ccccc8)c8ccc(-c9ccc%10ccccc%10c9)cc8)cc7)cc6-c6ccccc6)cc5c4o3)cc2)cc1]

Compound AK (15 g, 42.2 mmol) and Compound amine33 (25.1 g, 44.3 mmol) were added to THE (300 ml) under a nitrogen atmosphere, and the mixture was stirred and refluxed. Then, potassium carbonate (17.5 g, 126.5 mmol) was dissolved in water (52 ml), and then added thereto. Thereafter, it was stirred sufficiently, followed by adding bis(tri-tert-butylphosphine)palladium(0) (0.5 g, 1.0 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 21.3 g of Compound 34 (Yield 60%, MS: [M+H]⁺=843).

### Example 35: Preparation of Compound 35

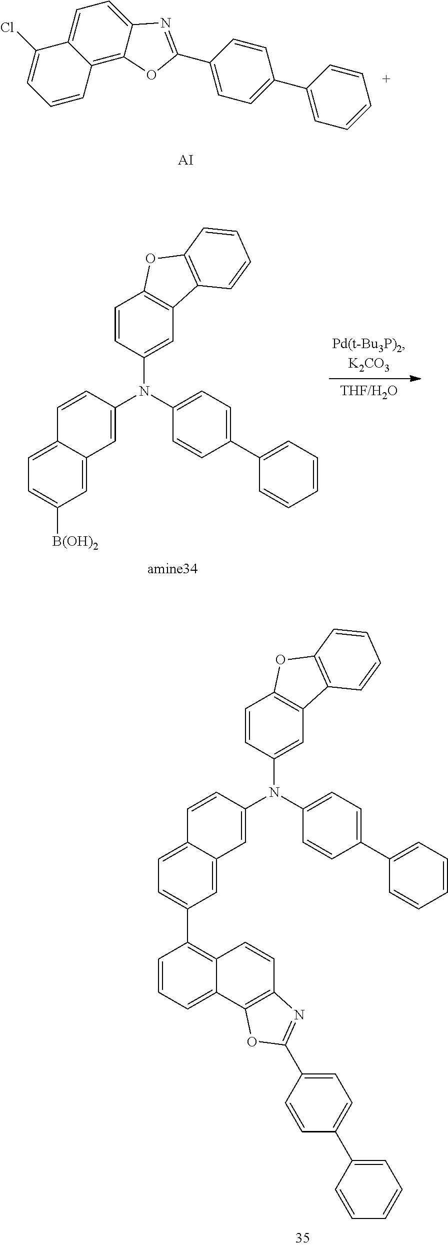 [SMILES: Clc1cccc2c1ccc1nc(-c3ccc(-c4ccccc4)cc3)oc12 ; "AI" ; OB(O)c1ccc2ccc(N(c3ccc(-c4ccccc4)cc3)c3ccc4oc5ccccc5c4c3)cc2c1 ; c1ccc(-c2ccc(-c3nc4ccc5c(-c6ccc7ccc(N(c8ccc(-c9ccccc9)cc8)c8ccc9oc%10ccccc%10c9c8)cc7c6)cccc5c4o3)cc2)cc1]

Compound AI (15 g, 42.2 mmol) and Compound amine34 (22.4 g, 44.3 mmol) were added to THE (300 ml) under a nitrogen atmosphere, and the mixture was stirred and refluxed. Then, potassium carbonate (17.5 g, 126.5 mmol) was dissolved in water (52 ml), and then added thereto. Thereafter, it was stirred sufficiently, followed by adding bis(tri-tert-butylphosphine)palladium(0) (0.5 g, 1.0 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 22.7 g of Compound 35 (Yield 69%, MS: [M+H]⁺=781).

### Example 36: Preparation of Compound 36

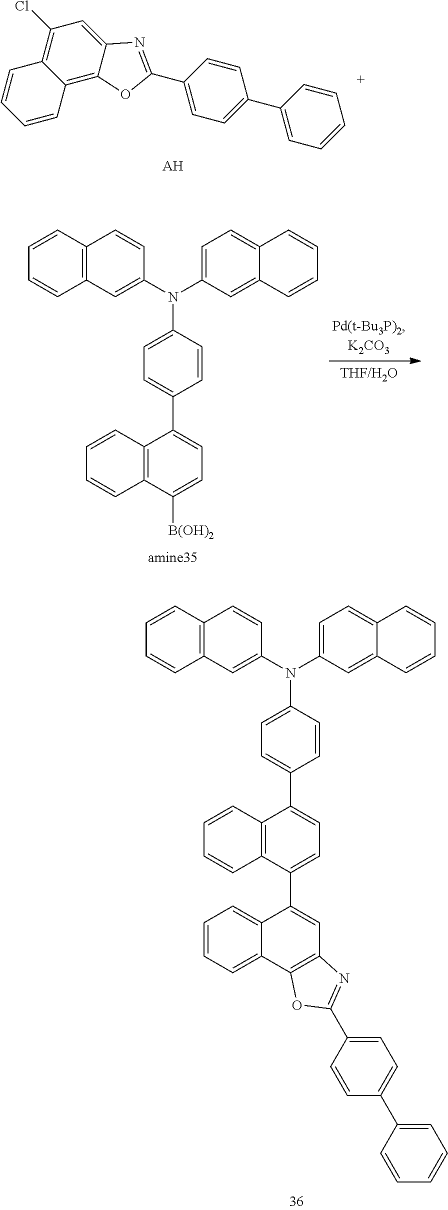 [SMILES: Clc1cc2nc(-c3ccc(-c4ccccc4)cc3)oc2c2ccccc12 ; "AH" ; OB(O)c1ccc(-c2ccc(N(c3ccc4ccccc4c3)c3ccc4ccccc4c3)cc2)c2ccccc12 ; c1ccc(-c2ccc(-c3nc4cc(-c5ccc(-c6ccc(N(c7ccc8ccccc8c7)c7ccc8ccccc8c7)cc6)c6ccccc56)c5ccccc5c4o3)cc2)cc1]

Compound AH (15 g, 42.2 mmol) and Compound amine35 (22.8 g, 44.3 mmol) were added to THE (300 ml) under a nitrogen atmosphere, and the mixture was stirred and refluxed. Then, potassium carbonate (17.5 g, 126.5 mmol) was dissolved in water (52 ml), and then added thereto. Thereafter, it was stirred sufficiently, followed by adding bis(tri-tert-butylphosphine)palladium(0) (0.5 g, 1.0 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 23 g of Compound 36 (Yield 69%, MS: [M+H]⁺=791).

### Example 37: Preparation of Compound 37

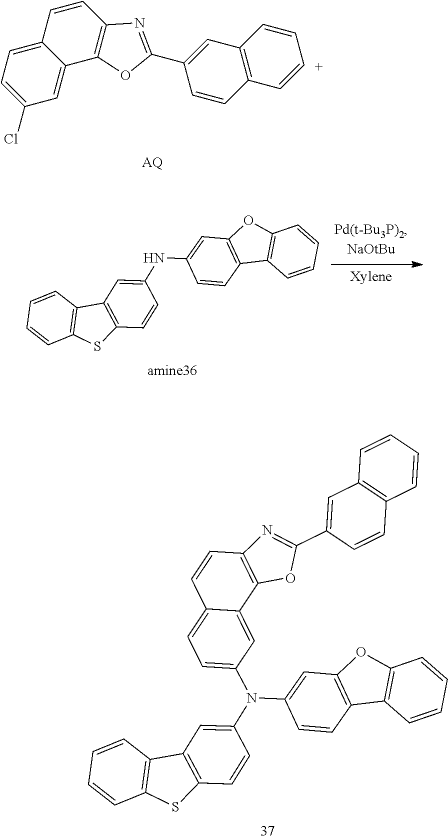

Compound AQ (10 g, 30.3 mmol), Compound amine36 (11.1 g, 30.3 mmol), and sodium tert-butoxide (9.7 g, 45.5 mmol) were added to xylene (200 ml) under a nitrogen atmosphere, and the mixture was stirred and refluxed. Thereafter, bis(tri-tert-butylphosphine)palladium(0) (0.2 g, 0.4 mmol) was added thereto. Upon completion of the reaction after 3 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 13.6 g of Compound 37 (Yield 68%, MS: [M+H]⁺=659).

### Example 38: Preparation of Compound 38

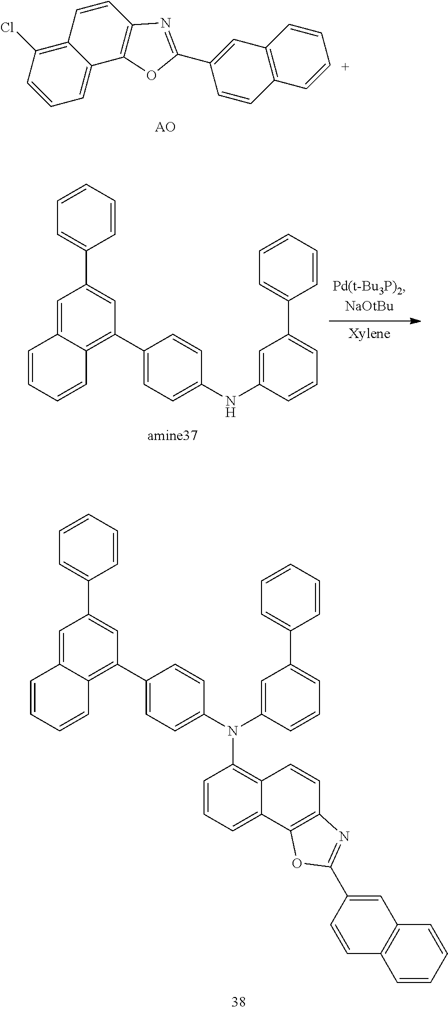 [SMILES: Clc1cccc2c1ccc1nc(-c3ccc4c(c3)C=C[C+]=C4)oc12 ; c1ccc(-c2cccc(Nc3ccc(-c4cc(-c5ccccc5)cc5ccccc45)cc3)c2)cc1 ; c1ccc(-c2cccc(N(c3ccc(-c4cc(-c5ccccc5)cc5ccccc45)cc3)c3cccc4c3ccc3nc(-c5ccc6ccccc6c5)oc34)c2)cc1]

Compound AO (10 g, 30.3 mmol), Compound amine37 (13.6 g, 30.3 mmol), and sodium tert-butoxide (9.7 g, 45.5 mmol) were added to xylene (200 ml) under a nitrogen atmosphere, and the mixture was stirred and refluxed. Thereafter, bis(tri-tert-butylphosphine)palladium(0) (0.2 g, 0.4 mmol) was added thereto. Upon completion of the reaction after 2 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 14.4 g of Compound 38 (Yield 64%, MS: [M+H]⁺=741).

### Example 39: Preparation of Compound 39

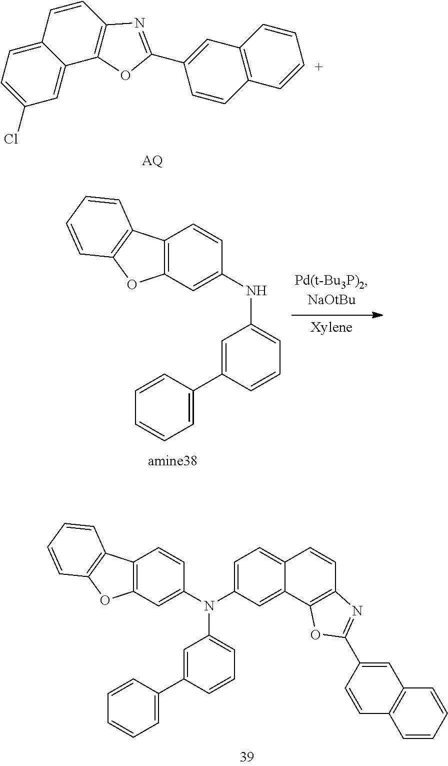

Compound AQ (10 g, 30.3 mmol), Compound amine38 (10.2 g, 30.3 mmol), and sodium tert-butoxide (9.7 g, 45.5 mmol) were added to xylene (200 ml) under a nitrogen atmosphere, and the mixture was stirred and refluxed. Thereafter, bis(tri-tert-butylphosphine)palladium(0) (0.2 g, 0.4 mmol) was added thereto. Upon completion of the reaction after 2 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 12 g of Compound 39 (Yield 63%, MS: [M+H]⁺=629).

### Example 40: Preparation of Compound 40

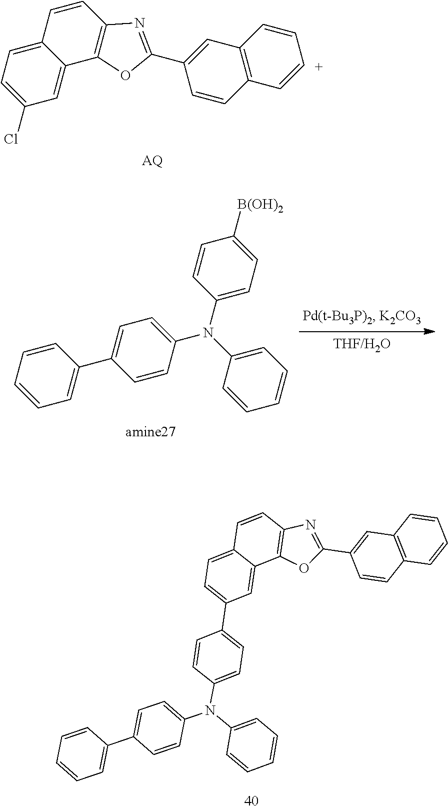

Compound AQ (15 g, 45.5 mmol) and Compound amine27 (17.4 g, 47.8 mmol) were added to THE (300 ml) under a nitrogen atmosphere, and the mixture was stirred and refluxed. Then, potassium carbonate (18.9 g, 136.5 mmol) was dissolved in water (57 ml), and then added thereto. Thereafter, it was stirred sufficiently, followed by adding bis(tri-tert-butylphosphine)palladium(0) (0.5 g, 1.0 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 18.4 g of Compound 40 (Yield 66%, MS: [M+H]⁺=615).

### Example 41: Preparation of Compound 41

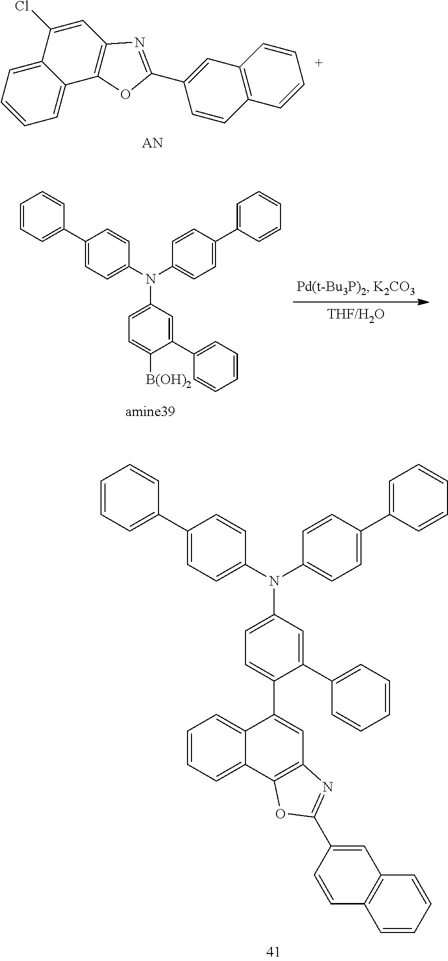 [SMILES: Clc1cc2nc(-c3ccc4ccccc4c3)oc2c2ccccc12 ; "AN" ; OB(O)c1ccc(N(c2ccc(-c3ccccc3)cc2)c2ccc(-c3ccccc3)cc2)cc1-c1ccccc1 ; c1ccc(-c2ccc(N(c3ccc(-c4ccccc4)cc3)c3ccc(-c4cc5nc(-c6ccc7ccccc7c6)oc5c5ccccc45)c(-c4ccccc4)c3)cc2)cc1]

Compound AN (15 g, 45.5 mmol) and Compound amine39 (24.7 g, 47.8 mmol) were added to THE (300 ml) under a nitrogen atmosphere, and the mixture was stirred and refluxed. Then, potassium carbonate (18.9 g, 136.5 mmol) was dissolved in water (57 ml), and then added thereto. Thereafter, it was stirred sufficiently, followed by adding bis(tri-tert-butylphosphine)palladium(0) (0.5 g, 1.0 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20.9 g of Compound 41 (Yield 60%, MS: [M+H]⁺=767).

### Example 42: Preparation of Compound 42

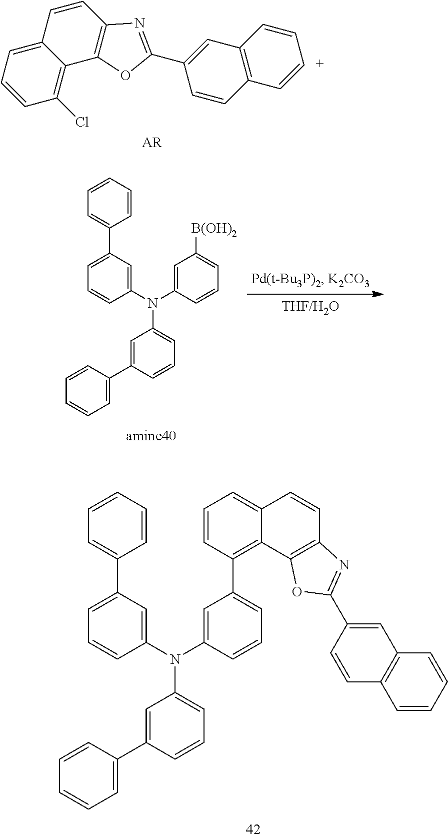

Compound AR (15 g, 45.5 mmol) and Compound amine40 (21.1 g, 47.8 mmol) were added to THE (300 ml) under a nitrogen atmosphere, and the mixture was stirred and refluxed. Then, potassium carbonate (18.9 g, 136.5 mmol) was dissolved in water (57 ml), and then added thereto. Thereafter, it was stirred sufficiently, followed by adding bis(tri-tert-butylphosphine)palladium(0) (0.5 g, 1.0 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20.7 g of Compound 42 (Yield 66%, MS: [M+H]⁺=691).

### Example 43: Preparation of Compound 43

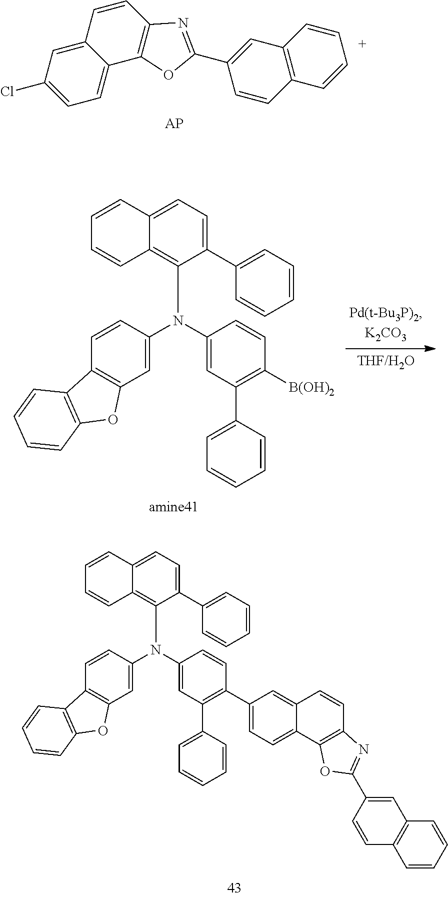 [SMILES: Clc1ccc2c(ccc3nc(-c4ccc5ccccc5c4)oc32)c1 ; "AP" ; OB(O)c1ccc(N(c2ccc3c(c2)oc2ccccc23)c2c(-c3ccccc3)ccc3ccccc23)cc1-c1ccccc1 ; c1ccc(-c2cc(N(c3ccc4c(c3)oc3ccccc34)c3c(-c4ccccc4)ccc4ccccc34)ccc2-c2ccc3c(ccc4nc(-c5ccc6ccccc6c5)oc43)c2)cc1]

Compound AP (15 g, 45.5 mmol) and Compound amine4l (27.8 g, 47.8 mmol) were added to THE (300 ml) under a nitrogen atmosphere, and the mixture was stirred and refluxed. Then, potassium carbonate (18.9 g, 136.5 mmol) was dissolved in water (57 ml), and then added thereto. Thereafter, it was stirred sufficiently, followed by adding bis(tri-tert-butylphosphine)palladium(0) (0.5 g, 1.0 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 26.1 g of Compound 43 (Yield 69%, MS: [M+H]⁺=831).

### Example 44: Preparation of Compound 44

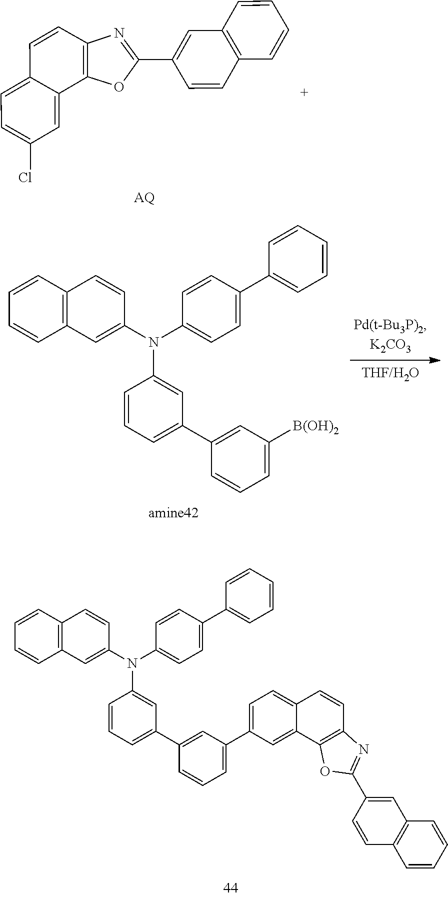

Compound AQ (15 g, 45.5 mmol) and Compound amine42 (23.5 g, 47.8 mmol) were added to THE (300 ml) under a nitrogen atmosphere, and the mixture was stirred and refluxed. Then, potassium carbonate (18.9 g, 136.5 mmol) was dissolved in water (57 ml), and then added thereto. Thereafter, it was stirred sufficiently, followed by adding bis(tri-tert-butylphosphine)palladium(0) (0.5 g, 1.0 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 22.9 g of Compound 44 (Yield 68%, MS: [M+H]⁺=741).

### Example 45: Preparation of Compound 45

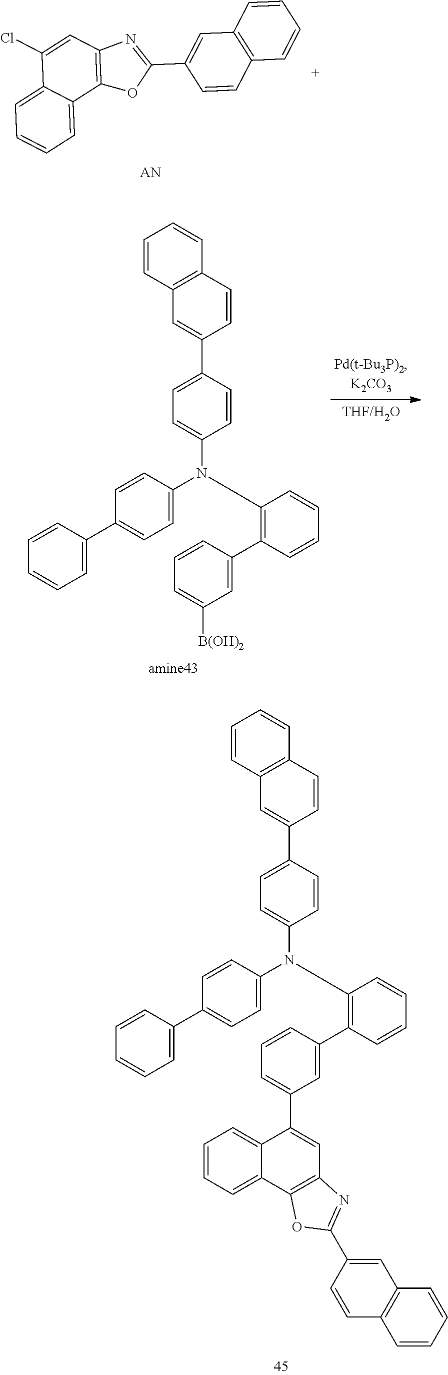 [SMILES: Clc1cc2nc(-c3ccc4ccccc4c3)oc2c2ccccc12 ; OB(O)c1cccc(-c2ccccc2N(c2ccc(-c3ccccc3)cc2)c2ccc(-c3ccc4ccccc4c3)cc2)c1 ; c1ccc(-c2ccc(N(c3ccc(-c4ccc5ccccc5c4)cc3)c3ccccc3-c3cccc(-c4cc5nc(-c6ccc7ccccc7c6)oc5c5ccccc45)c3)cc2)cc1]

Compound AN (15 g, 45.5 mmol) and Compound amine43 (27.1 g, 47.8 mmol) were added to THE (300 ml) under a nitrogen atmosphere, and the mixture was stirred and refluxed. Then, potassium carbonate (18.9 g, 136.5 mmol) was dissolved in water (57 ml), and then added thereto. Thereafter, it was stirred sufficiently, followed by adding bis(tri-tert-butylphosphine)palladium(0) (0.5 g, 1.0 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 26 g of Compound 45 (Yield 70%, MS: [M+H]⁺=817).

### Example 46: Preparation of Compound 46

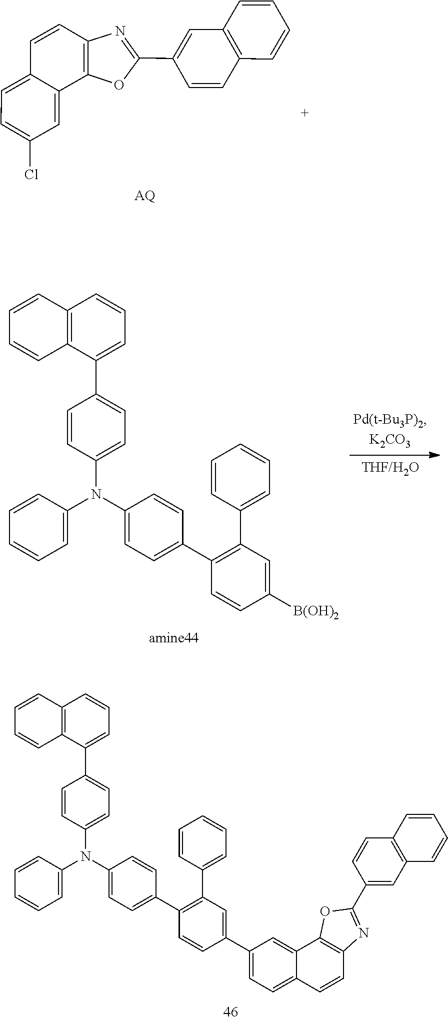

Compound AQ (15 g, 45.5 mmol) and Compound amine44 (27.1 g, 47.8 mmol) were added to THE (300 ml) under a nitrogen atmosphere, and the mixture was stirred and refluxed. Then, potassium carbonate (18.9 g, 136.5 mmol) was dissolved in water (57 ml), and then added thereto. Thereafter, it was stirred sufficiently, followed by adding bis(tri-tert-butylphosphine)palladium(0) (0.5 g, 1.0 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 22.3 g of Compound 46 (Yield 60%, MS: [M+H]⁺=817).

### Example 47: Preparation of Compound 47

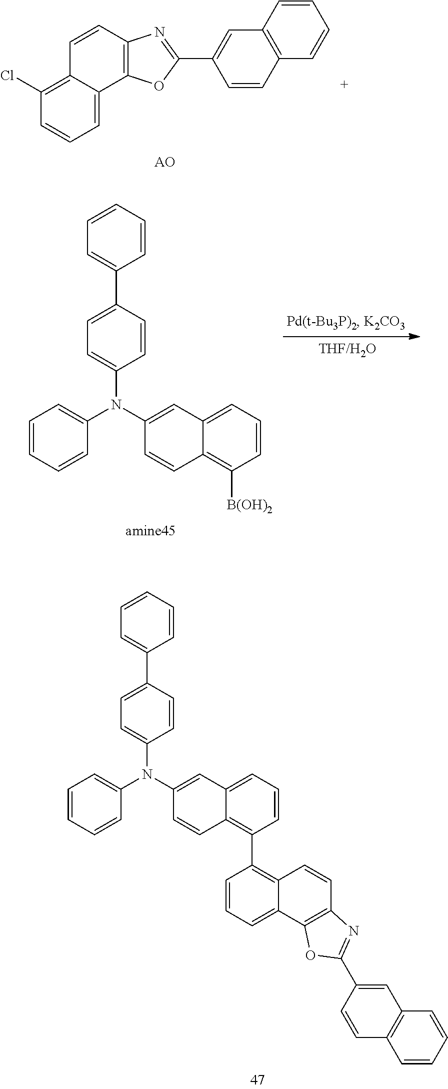 [SMILES: Clc1cccc2c1ccc1nc(-c3ccc4ccccc4c3)oc12 ; OB(O)c1cccc2cc(N(c3ccccc3)c3ccc(-c4ccccc4)cc3)ccc12 ; c1ccc(-c2ccc(N(c3ccccc3)c3ccc4c(-c5cccc6c5ccc5nc(-c7ccc8ccccc8c7)oc56)cccc4c3)cc2)cc1]

Compound AQ (15 g, 43.4 mmol) and Compound amine45 (25.8 g, 45.5 mmol) were added to THE (300 ml) under a nitrogen atmosphere, and the mixture was stirred and refluxed. Then, potassium carbonate (18 g, 130.1 mmol) was dissolved in water (54 ml), and then added thereto. Thereafter, it was stirred sufficiently, followed by adding bis(tri-tert-butylphosphine)palladium(0) (0.5 g, 1.0 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 19.5 g of Compound 47 (Yield 54%, MS: [M+H]⁺=833).

### Example 48: Preparation of Compound 48

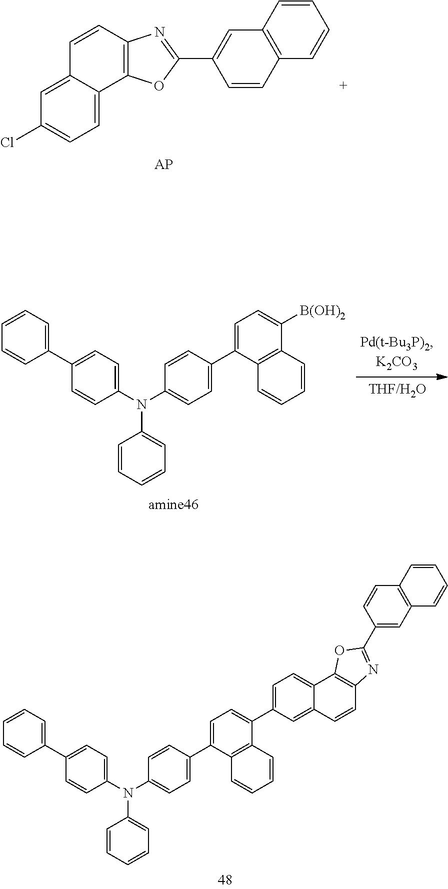 [SMILES: Clc1ccc2c(ccc3nc(-c4ccc5ccccc5c4)oc32)c1 ; OB(O)c1ccc(-c2ccc(N(c3ccccc3)c3ccc(-c4ccccc4)cc3)cc2)c2ccccc12 ; c1ccc(-c2ccc(N(c3ccccc3)c3ccc(-c4ccc(-c5ccc6c(ccc7nc(-c8ccc9ccccc9c8)oc76)c5)c5ccccc45)cc3)cc2)cc1]

Compound AP (15 g, 45.5 mmol) and Compound amine46 (23.5 g, 47.8 mmol) were added to THE (300 ml) under a nitrogen atmosphere, and the mixture was stirred and refluxed. Then, potassium carbonate (18.9 g, 136.5 mmol) was dissolved in water (57 ml), and then added thereto. Thereafter, it was stirred sufficiently, followed by adding bis(tri-tert-butylphosphine)palladium(0) (0.5 g, 1.0 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20.2 g of Compound 48 (Yield 60%, MS: [M+H]⁺=741).

### Example 49: Preparation of Compound 49

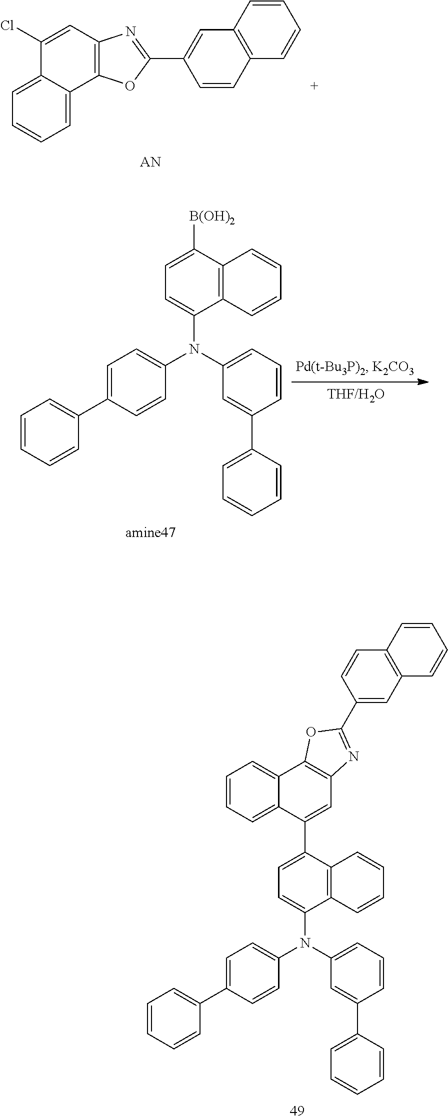 [SMILES: Clc1cc2nc(-c3ccc4ccccc4c3)oc2c2ccccc12 ; OB(O)c1ccc(N(c2ccc(-c3ccccc3)cc2)c2cccc(-c3ccccc3)c2)c2ccccc12 ; c1ccc(-c2ccc(N(c3cccc(-c4ccccc4)c3)c3ccc(-c4cc5nc(-c6ccc7ccccc7c6)oc5c5ccccc45)c4ccccc34)cc2)cc1]

Compound AN (15 g, 45.5 mmol) and Compound amine47 (23.5 g, 47.8 mmol) were added to THE (300 ml) under a nitrogen atmosphere, and the mixture was stirred and refluxed. Then, potassium carbonate (18.9 g, 136.5 mmol) was dissolved in water (57 ml), and then added thereto. Thereafter, it was stirred sufficiently, followed by adding bis(tri-tert-butylphosphine)palladium(0) (0.5 g, 1.0 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 21.5 g of Compound 49 (Yield 64%, MS: [M+H]⁺=741).

### Example 50: Preparation of Compound 50

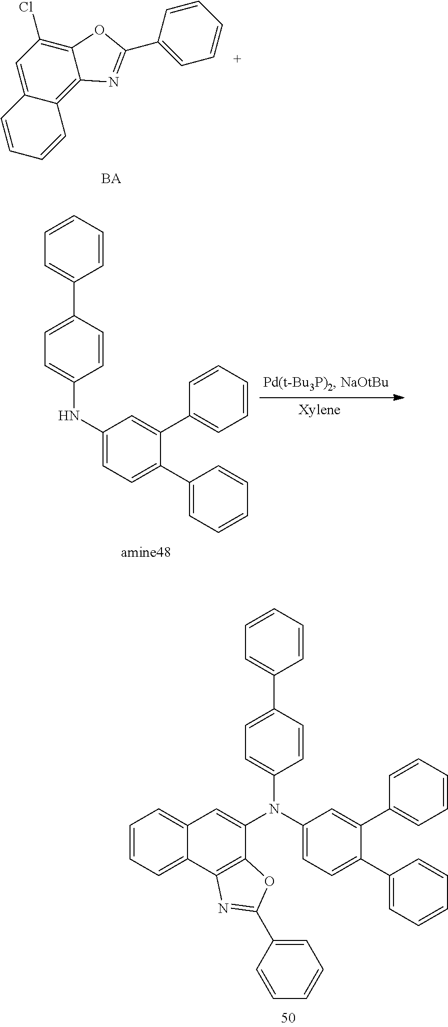 [SMILES: Clc1cc2ccccc2c2nc(-c3ccccc3)oc12 ; c1ccc(-c2ccc(Nc3ccc(-c4ccccc4)c(-c4ccccc4)c3)cc2)cc1 ; c1ccc(-c2ccc(N(c3ccc(-c4ccccc4)c(-c4ccccc4)c3)c3cc4ccccc4c4nc(-c5ccccc5)oc34)cc2)cc1]

Compound BA (10 g, 30.3 mmol), Compound amine48 (12.1 g, 30.3 mmol), and sodium tert-butoxide (9.7 g, 45.5 mmol) were added to xylene (200 ml) under a nitrogen atmosphere, and the mixture was stirred and refluxed. Thereafter, bis(tri-tert-butylphosphine)palladium(0) (0.2 g, 0.4 mmol) was added thereto. Upon completion of the reaction after 2 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 11.8 g of Compound 50 (Yield 61%, MS: [M+H]⁺=641).

### Example 51: Preparation of Compound 51

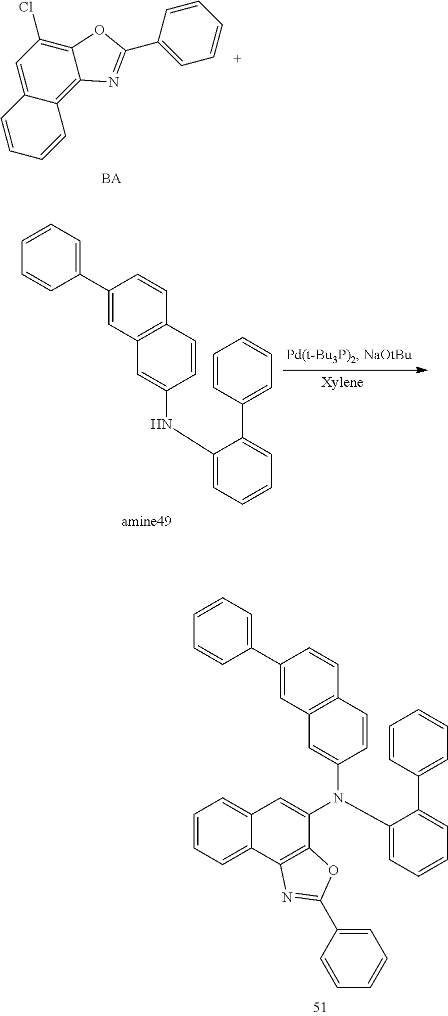 [SMILES: Clc1cc2ccccc2c2nc(-c3ccccc3)oc12 ; c1ccc(-c2ccc3ccc(Nc4ccccc4-c4ccccc4)cc3c2)cc1 ; c1ccc(-c2ccc3ccc(N(c4ccccc4-c4ccccc4)c4cc5ccccc5c5nc(-c6ccccc6)oc45)cc3c2)cc1]

Compound BA (10 g, 30.3 mmol), Compound amine49 (11.3 g, 30.3 mmol), and sodium tert-butoxide (9.7 g, 45.5 mmol) were added to xylene (200 ml) under a nitrogen atmosphere, and the mixture was stirred and refluxed. Thereafter, bis(tri-tert-butylphosphine)palladium(0) (0.2 g, 0.4 mmol) was added thereto. Upon completion of the reaction after 3 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 11.7 g of Compound 51 (Yield 63%, MS: [M+H]⁺=615).

### Example 52: Preparation of Compound 52

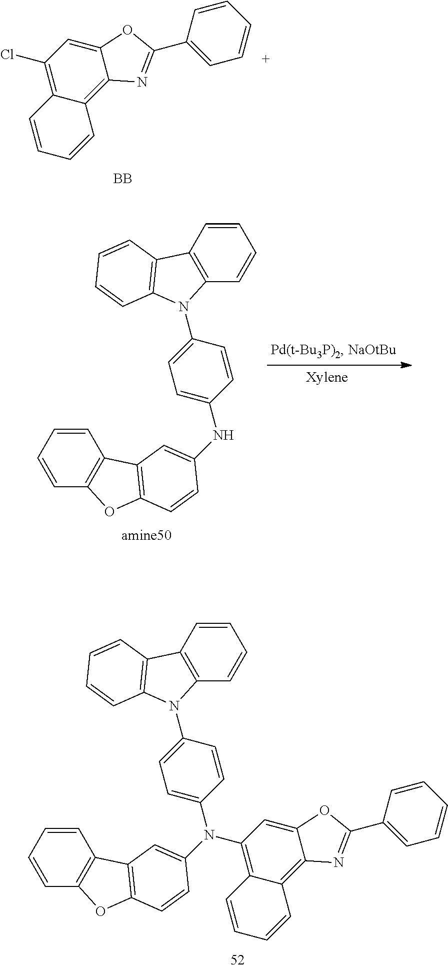

Compound BB (10 g, 30.3 mmol), Compound amine50 (12.9 g, 30.3 mmol), and sodium tert-butoxide (9.7 g, 45.5 mmol) were added to xylene (200 ml) under a nitrogen atmosphere, and the mixture was stirred and refluxed. Thereafter, bis(tri-tert-butylphosphine)palladium(0) (0.2 g, 0.4 mmol) was added thereto. Upon completion of the reaction after 2 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 14 g of Compound 52 (Yield 69%, MS: [M+H]⁺=668).

### Example 53: Preparation of Compound 53

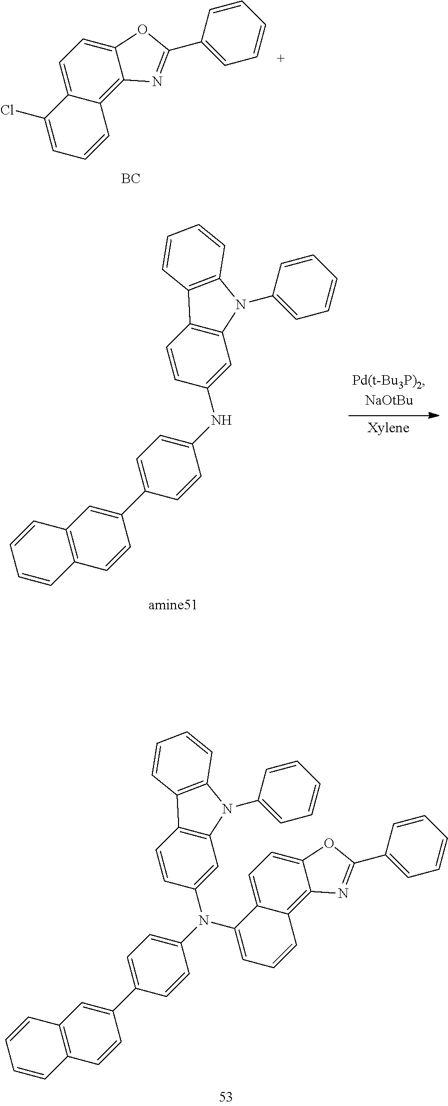

Compound BC (10 g, 30.3 mmol), Compound amine51 (14 g, 30.3 mmol), and sodium tert-butoxide (9.7 g, 45.5 mmol) were added to xylene (200 ml) under a nitrogen atmosphere, and the mixture was stirred and refluxed. Thereafter, bis(tri-tert-butylphosphine)palladium(0) (0.2 g, 0.4 mmol) was added thereto. Upon completion of the reaction after 2 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 12.8 g of Compound 53 (Yield 60%, MS: [M+H]⁺=704).

### Example 54: Preparation of Compound 54

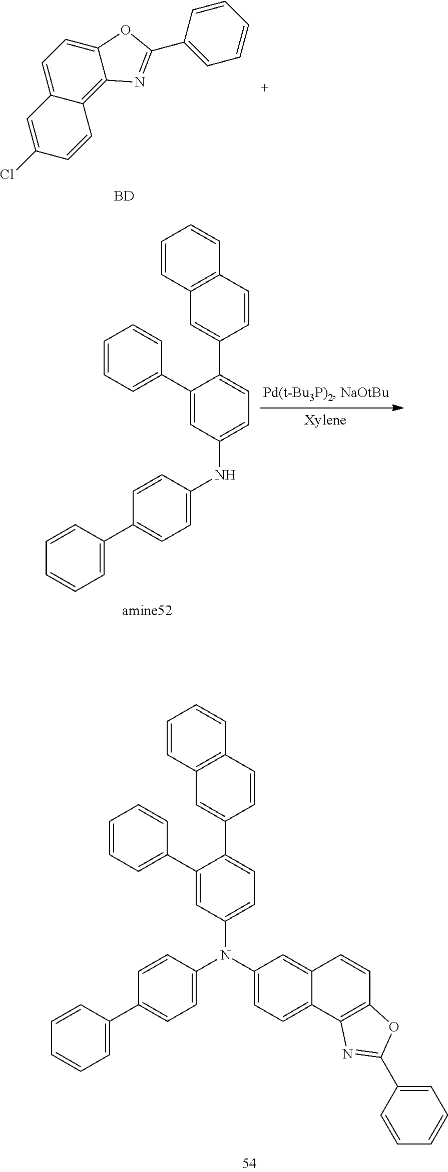 [SMILES: Clc1ccc2c(ccc3oc(-c4ccccc4)nc32)c1 ; "BD" ; c1ccc(-c2ccc(Nc3ccc(-c4ccc5ccccc5c4)c(-c4ccccc4)c3)cc2)cc1 ; c1ccc(-c2ccc(N(c3ccc(-c4ccc5ccccc5c4)c(-c4ccccc4)c3)c3ccc4c(ccc5oc(-c6ccccc6)nc54)c3)cc2)cc1]

Compound BD (10 g, 30.3 mmol), Compound amine52 (13.6 g, 30.3 mmol), and sodium tert-butoxide (9.7 g, 45.5 mmol) were added to xylene (200 ml) under a nitrogen atmosphere, and the mixture was stirred and refluxed. Thereafter, bis(tri-tert-butylphosphine)palladium(0) (0.2 g, 0.4 mmol) was added thereto. Upon completion of the reaction after 2 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 12.6 g of Compound 54 (Yield 60%, MS: [M+H]⁺=691).

### Example 55: Preparation of Compound 55

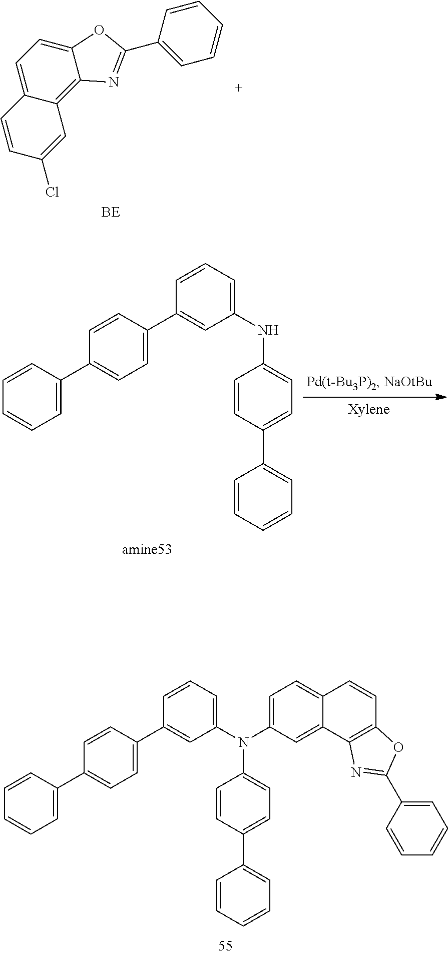 [SMILES: Clc1ccc2ccc3oc(-c4ccccc4)nc3c2c1 ; "BE" ; c1ccc(-c2ccc(Nc3cccc(-c4ccc(-c5ccccc5)cc4)c3)cc2)cc1 ; c1ccc(-c2ccc(-c3cccc(N(c4ccc(-c5ccccc5)cc4)c4ccc5ccc6oc(-c7ccccc7)nc6c5c4)c3)cc2)cc1]

Compound BE (10 g, 30.3 mmol), Compound amine53 (12.1 g, 30.3 mmol), and sodium tert-butoxide (9.7 g, 45.5 mmol) were added to xylene (200 ml) under a nitrogen atmosphere, and the mixture was stirred and refluxed. Thereafter, bis(tri-tert-butylphosphine)palladium(0) (0.2 g, 0.4 mmol) was added thereto. Upon completion of the reaction after 2 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 13.2 g of Compound 55 (Yield 68%, MS: [M+H]⁺=641).

### Example 56: Preparation of Compound 56

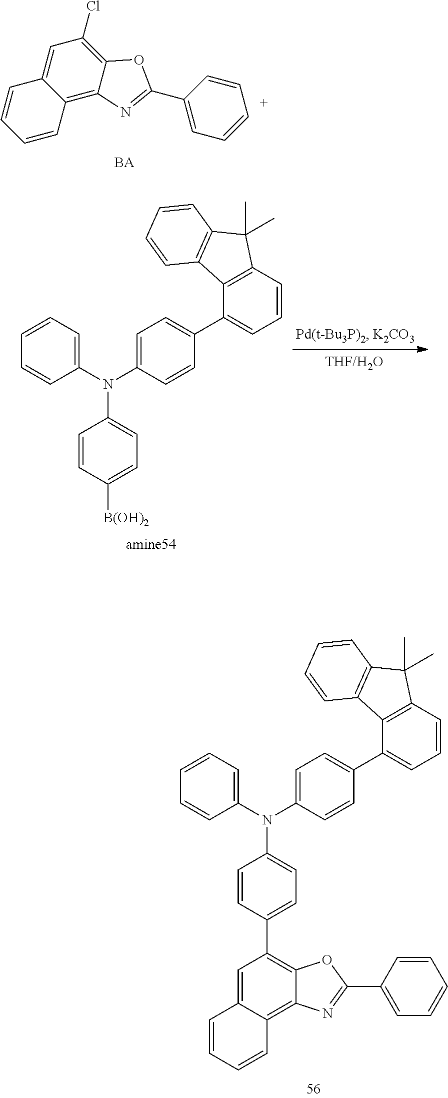 [SMILES: Clc1cc2ccccc2c2nc(-c3ccccc3)oc12 ; CC1(C)c2ccccc2-c2c(-c3ccc(N(c4ccccc4)c4ccc(B(O)O)cc4)cc3)cccc21 ; CC1(C)c2ccccc2-c2c(-c3ccc(N(c4ccccc4)c4ccc(-c5cc6ccccc6c6nc(-c7ccccc7)oc56)cc4)cc3)cccc21]

Compound BA (15 g, 53.6 mmol) and Compound amine54 (27.1 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 21.9 g of Compound 56 (Yield 60%, MS: [M+H]⁺=681).

### Example 57: Preparation of Compound 57

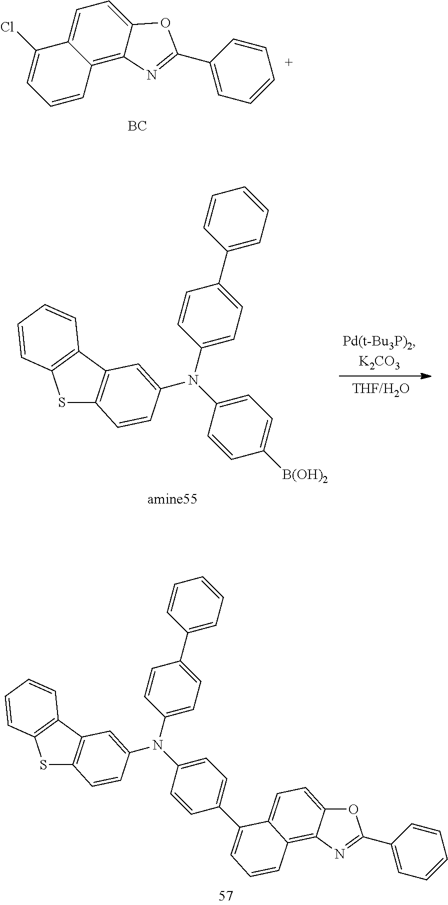

Compound BC (15 g, 53.6 mmol) and Compound amine55 (26.5 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 23 g of Compound 57 (Yield 64%, MS: [M+H]⁺=671).

### Example 58: Preparation of Compound 58

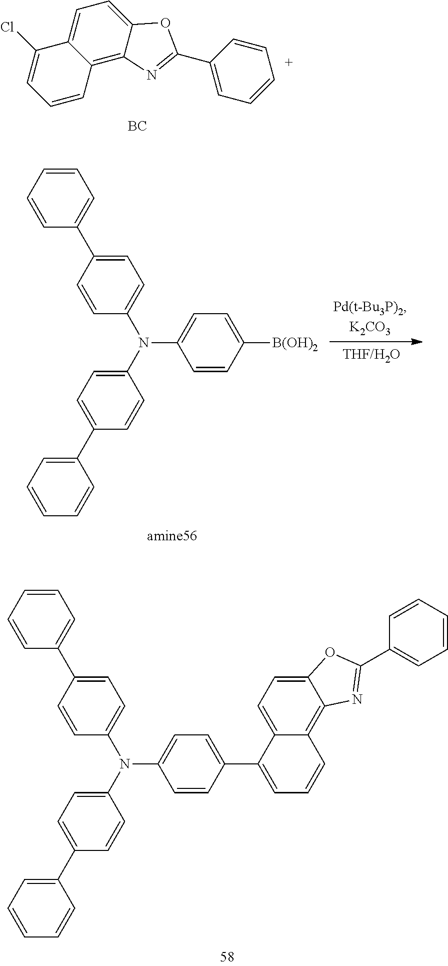

Compound BC (15 g, 53.6 mmol) and Compound amine56 (24.9 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 22.7 g of Compound 58 (Yield 66%, MS: [M+H]⁺=641).

### Example 59: Preparation of Compound 59

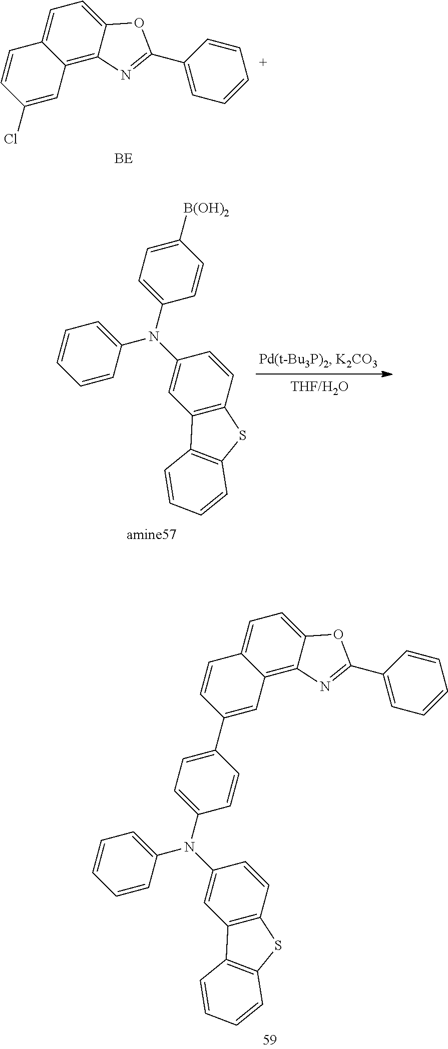 [SMILES: Clc1ccc2ccc3oc(-c4ccccc4)nc3c2c1 ; "BE" ; OB(O)c1ccc(N(c2ccccc2)c2ccc3sc4ccccc4c3c2)cc1 ; c1ccc(-c2nc3c(ccc4ccc(-c5ccc(N(c6ccccc6)c6ccc7sc8ccccc8c7c6)cc5)cc43)o2)cc1]

Compound BE (15 g, 53.6 mmol) and Compound amine57 (22.3 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 22.3 g of Compound 59 (Yield 70%, MS: [M+H]⁺=595).

### Example 60: Preparation of Compound 60

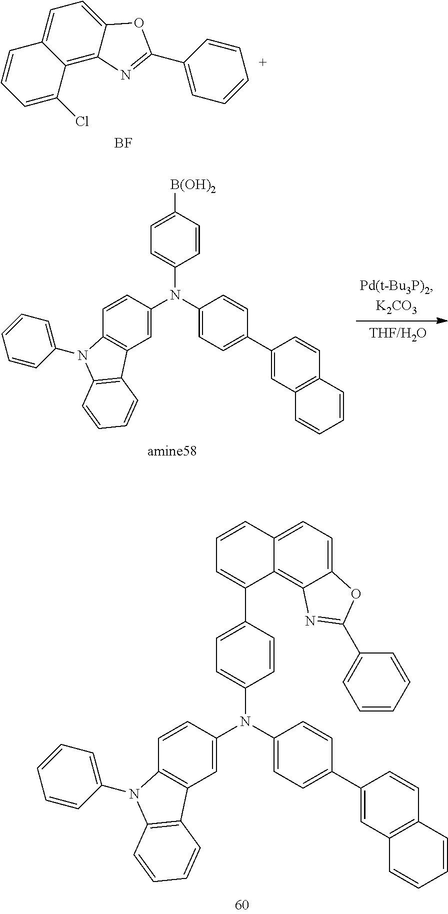

Compound BF (15 g, 53.6 mmol) and Compound amine58 (32.7 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 25.5 g of Compound 60 (Yield 61%, MS: [M+H]⁺=780).

### Example 61: Preparation of Compound 61

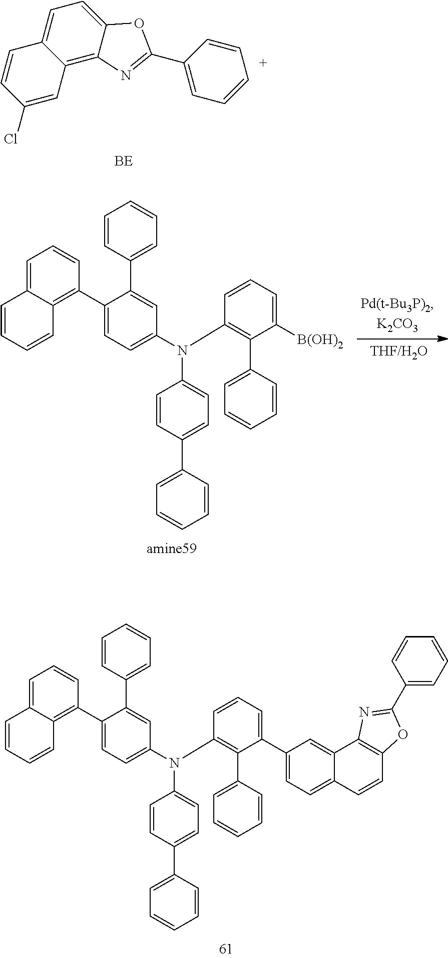

Compound BE (15 g, 53.6 mmol) and Compound amine59 (36.2 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 31.6 g of Compound 61 (Yield 70%, MS: [M+H]⁺=843).

### Example 62: Preparation of Compound 62

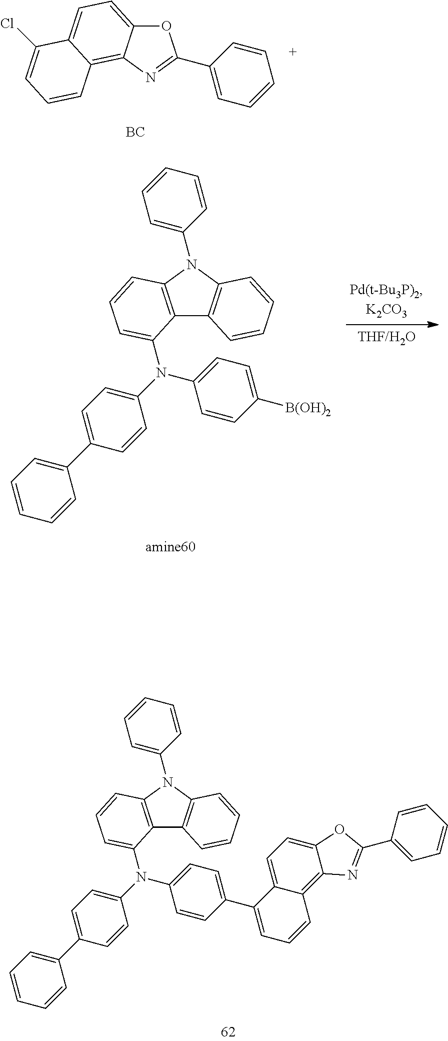 [SMILES: Clc1cccc2c1ccc1oc(-c3ccccc3)nc12 ; "BC" ; OB(O)c1ccc(N(c2ccc(-c3ccccc3)cc2)c2cccc3c2c2ccccc2n3-c2ccccc2)cc1 ; c1ccc(-c2ccc(N(c3ccc(-c4cccc5c4ccc4oc(-c6ccccc6)nc45)cc3)c3cccc4c3c3ccccc3n4-c3ccccc3)cc2)cc1]

Compound BC (15 g, 53.6 mmol) and Compound amine60 (29.9 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 26.2 g of Compound 62 (Yield 67%, MS: [M+H]⁺=730).

### Example 63: Preparation of Compound 63

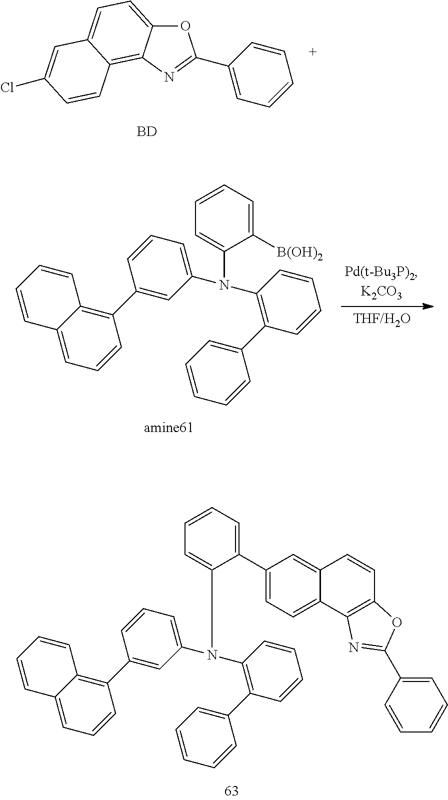

Compound BD (15 g, 53.6 mmol) and Compound amine61 (27.7 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 22.2 g of Compound 63 (Yield 60%, MS: [M+H]⁺=691).

### Example 64: Preparation of Compound 64

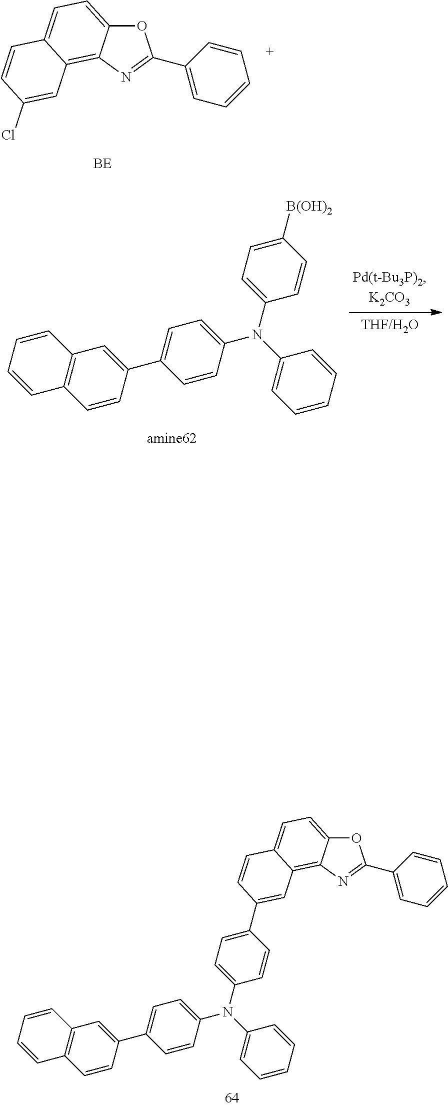 [SMILES: Clc1ccc2ccc3oc(-c4ccccc4)nc3c2c1 ; "BE" ; OB(O)c1ccc(N(c2ccccc2)c2ccc(-c3ccc4ccccc4c3)cc2)cc1 ; c1ccc(-c2nc3c(ccc4ccc(-c5ccc(N(c6ccccc6)c6ccc(-c7ccc8ccccc8c7)cc6)cc5)cc43)o2)cc1]

Compound BE (15 g, 53.6 mmol) and Compound amine62 (23.4 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 21.1 g of Compound 64 (Yield 64%, MS: [M+H]⁺=615).

### Example 65: Preparation of Compound 65

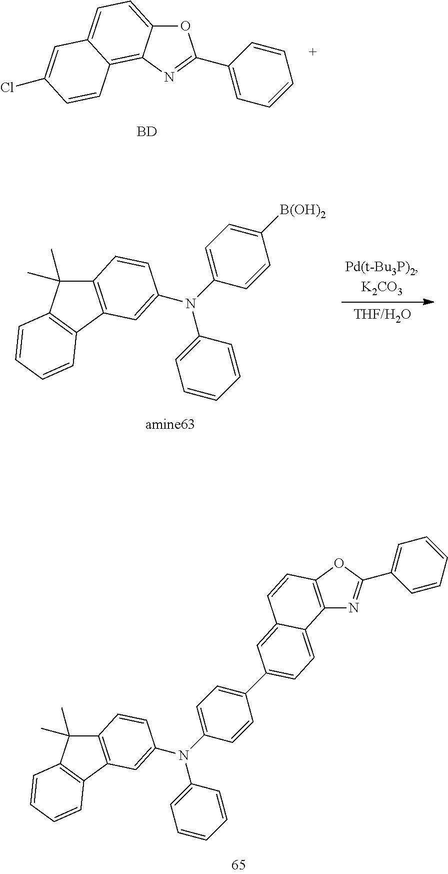

Compound BD (15 g, 53.6 mmol) and Compound amine63 (22.8 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 21.7 g of Compound 65 (Yield 67%, MS: [M+H]⁺=605).

### Example 66: Preparation of Compound 66

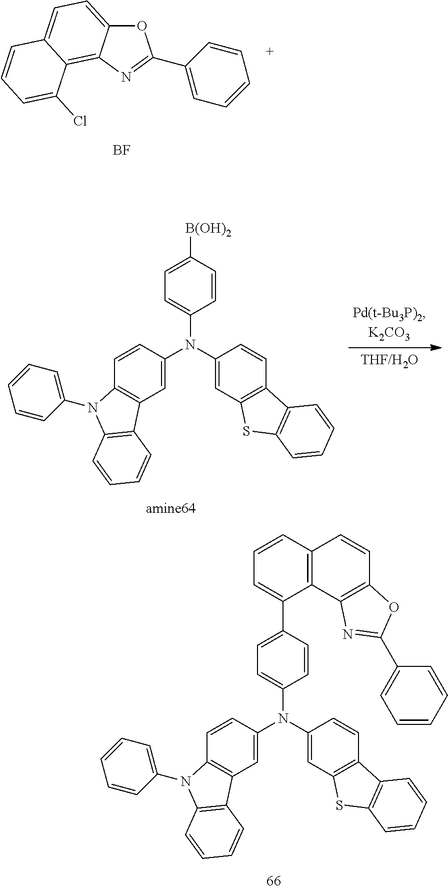

Compound BF (15 g, 53.6 mmol) and Compound amine64 (31.6 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 26.9 g of Compound 66 (Yield 66%, MS: [M+H]⁺=760).

### Example 67: Preparation of Compound 67

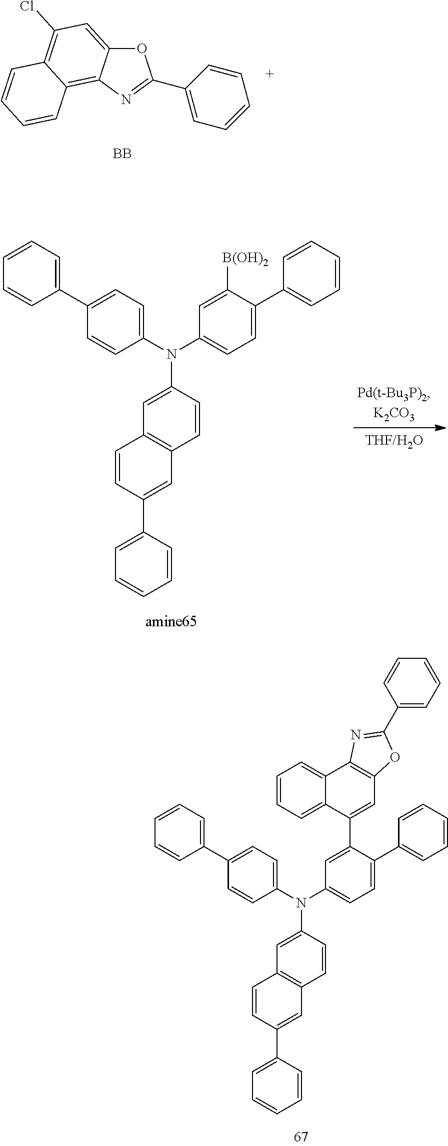 [SMILES: Clc1cc2oc(-c3ccccc3)nc2c2ccccc12 ; "BB" ; OB(O)c1cc(N(c2ccc(-c3ccccc3)cc2)c2ccc3cc(-c4ccccc4)ccc3c2)ccc1-c1ccccc1 ; c1ccc(-c2ccc(N(c3ccc(-c4ccccc4)c(-c4cc5oc(-c6ccccc6)nc5c5ccccc45)c3)c3ccc4cc(-c5ccccc5)ccc4c3)cc2)cc1]

Compound BB (15 g, 53.6 mmol) and Compound amine65 (32 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 27.1 g of Compound 67 (Yield 66%, MS: [M+H]⁺=767).

### Example 68: Preparation of Compound 68

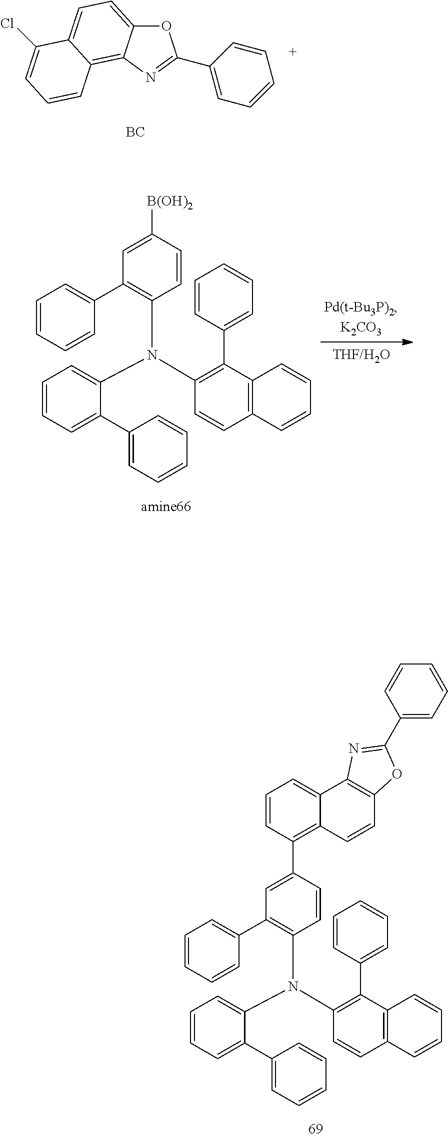 [SMILES: Clc1cccc2c1ccc1oc(-c3ccccc3)nc12 ; "BC" ; OB(O)c1ccc(N(c2ccccc2-c2ccccc2)c2ccc3ccccc3c2-c2ccccc2)c(-c2ccccc2)c1 ; c1ccc(-c2nc3c(ccc4c(-c5ccc(N(c6ccccc6-c6ccccc6)c6ccc7ccccc7c6-c6ccccc6)c(-c6ccccc6)c5)cccc43)o2)cc1]

Compound BC (15 g, 53.6 mmol) and Compound amine66 (32 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 19.8 g of Compound 68 (Yield 65%, MS: [M+H]⁺=569).

### Example 69: Preparation of Compound 69

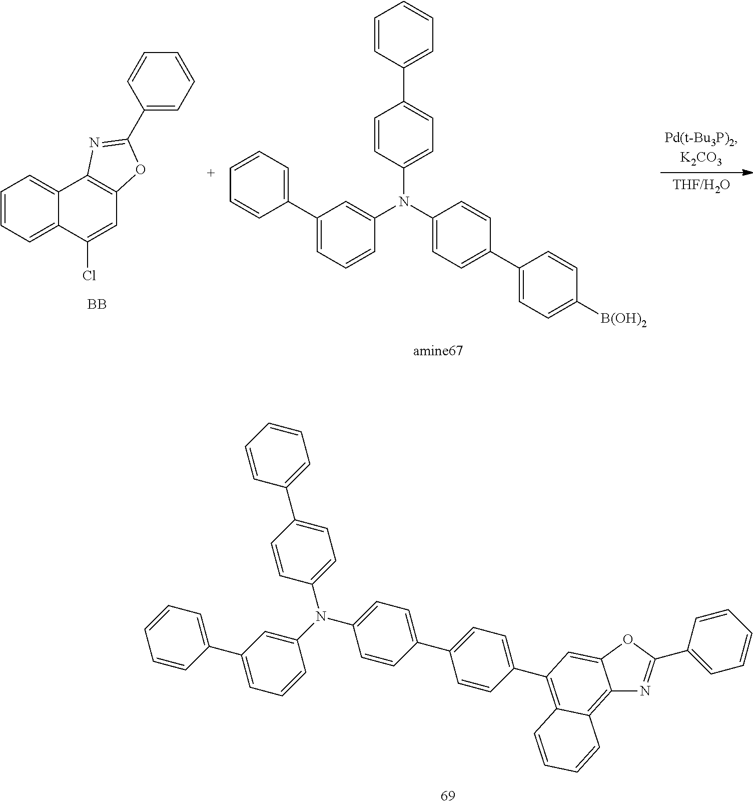

Compound BB (15 g, 53.6 mmol) and Compound amine67 (29.1 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 26.5 g of Compound 69 (Yield 69%, MS: [M+H]⁺=717).

### Example 70: Preparation of Compound 70

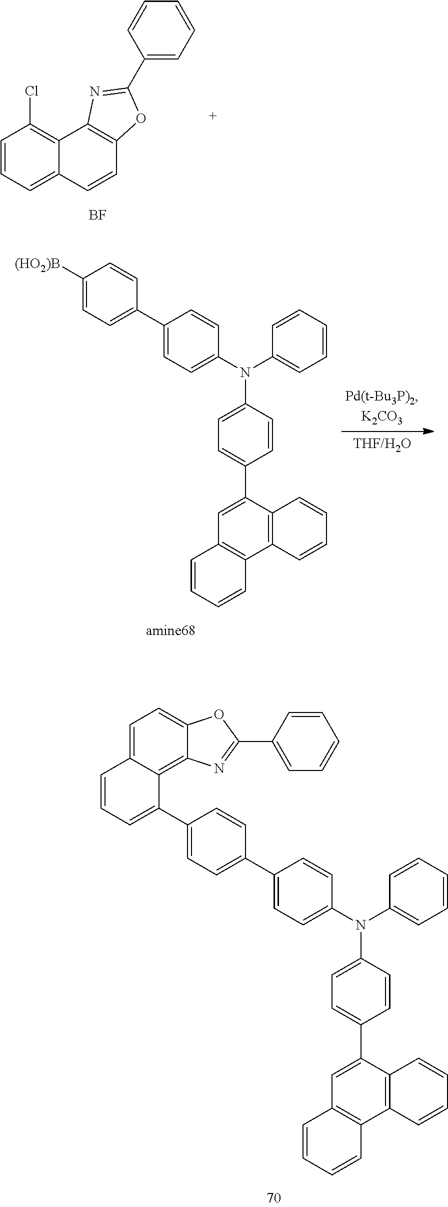

Compound BF (15 g, 53.6 mmol) and Compound amine68 (30.5 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 25 g of Compound 70 (Yield 63%, MS: [M+H]⁺=741).

### Example 71: Preparation of Compound 71

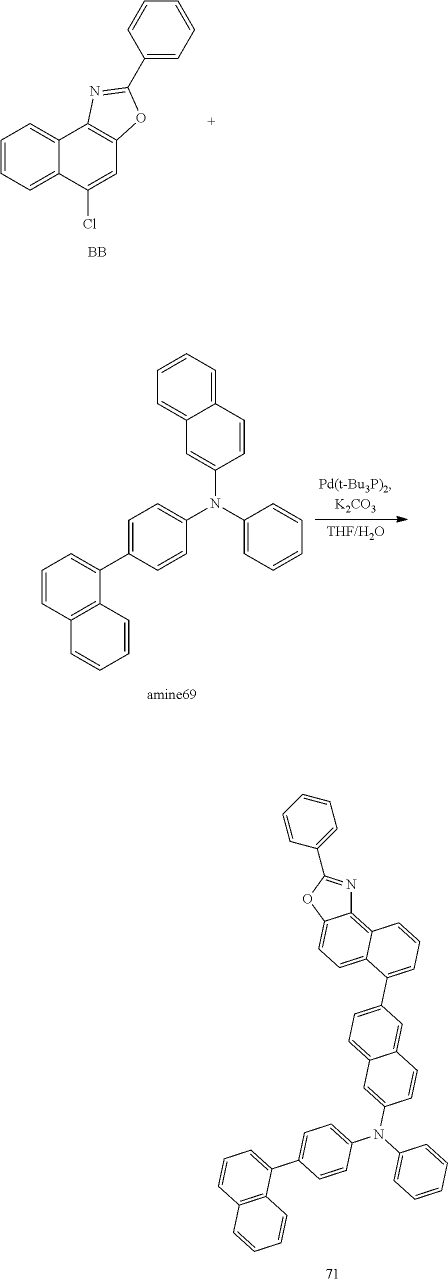

Compound BC (15 g, 53.6 mmol) and Compound amine69 (26.2 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 22.1 g of Compound 71 (Yield 62%, MS: [M+H]⁺=665).

### Example 72: Preparation of Compound 72

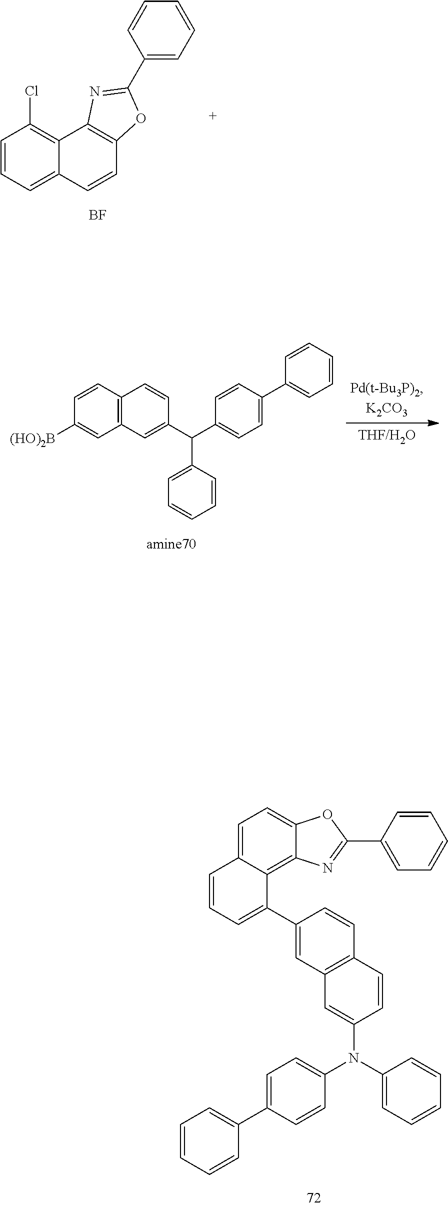

Compound BF (15 g, 53.6 mmol) and Compound amine70 (23.4 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 21.7 g of Compound 72 (Yield 66%, MS: [M+H]⁺=615).

### Example 73: Preparation of Compound 73

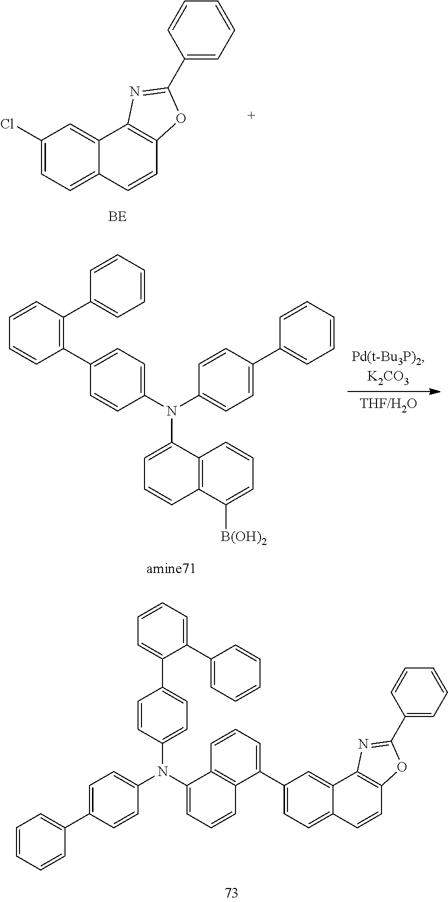 [SMILES: Clc1ccc2ccc3oc(-c4ccccc4)nc3c2c1 ; OB(O)c1cccc2c(N(c3ccc(-c4ccccc4)cc3)c3ccc(-c4ccccc4-c4ccccc4)cc3)cccc12 ; c1ccc(-c2ccc(N(c3ccc(-c4ccccc4-c4ccccc4)cc3)c3cccc4c(-c5ccc6ccc7oc(-c8ccccc8)nc7c6c5)cccc34)cc2)cc1]

Compound BE (15 g, 53.6 mmol) and Compound amine71 (32 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 25.5 g of Compound 73 (Yield 62%, MS: [M+H]⁺=767).

### Example 74: Preparation of Compound 74

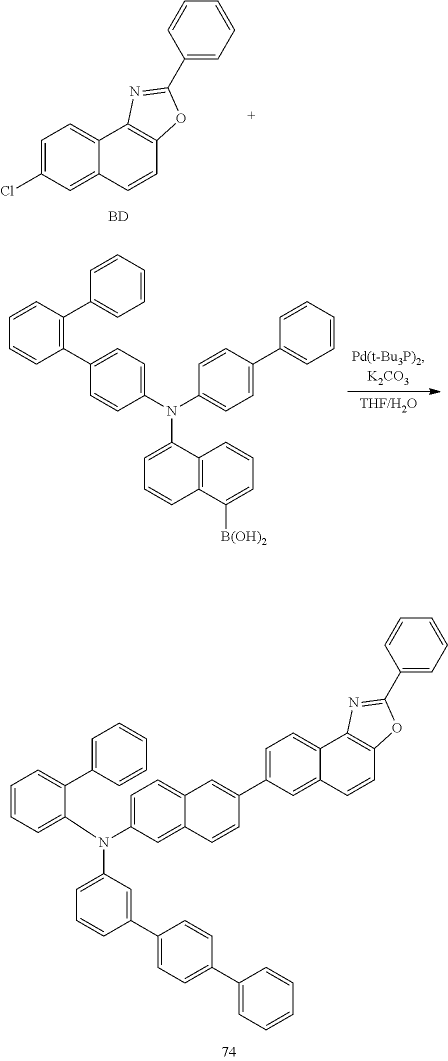

Compound BD (15 g, 53.6 mmol) and Compound amine72 (36.2 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 30.7 g of Compound 74 (Yield 68%, MS: [M+H]⁺=843).

### Example 75: Preparation of Compound 75

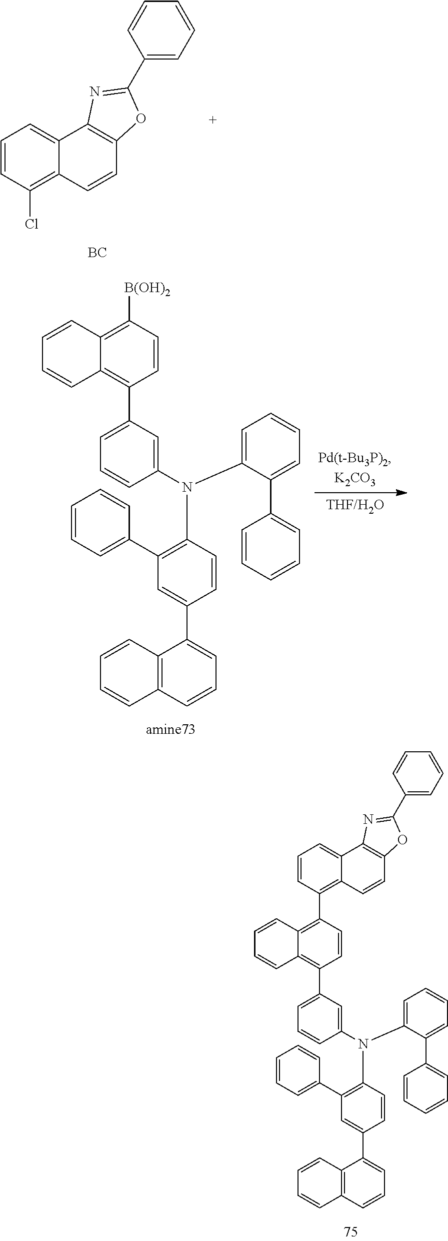 [SMILES: Clc1cccc2c1ccc1oc(-c3ccccc3)nc12 ; "BC" ; OB(O)c1ccc(-c2cccc(N(c3ccccc3-c3ccccc3)c3ccc(-c4cccc5ccccc45)cc3-c3ccccc3)c2)c2ccccc12 ; c1ccc(-c2nc3c(ccc4c(-c5ccc(-c6cccc(N(c7ccccc7-c7ccccc7)c7ccc(-c8cccc9ccccc89)cc7-c7ccccc7)c6)c6ccccc56)cccc43)o2)cc1]

Compound BC (15 g, 53.6 mmol) and Compound amine73 (39.1 g, 56.3 mmol) were added to THE (300 ml) under a nitrogen atmosphere, and the mixture was stirred and refluxed. Then, potassium carbonate (22.2 g, 160.9 mmol) was dissolved in water (67 ml), and then added thereto. Thereafter, it was stirred sufficiently, followed by adding bis(tri-tert-butylphosphine)palladium(0) (0.6 g, 1.2 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 29.7 g of Compound 75 (Yield 62%, MS: [M+H]⁺=893).

### Example 76: Preparation of Compound 76

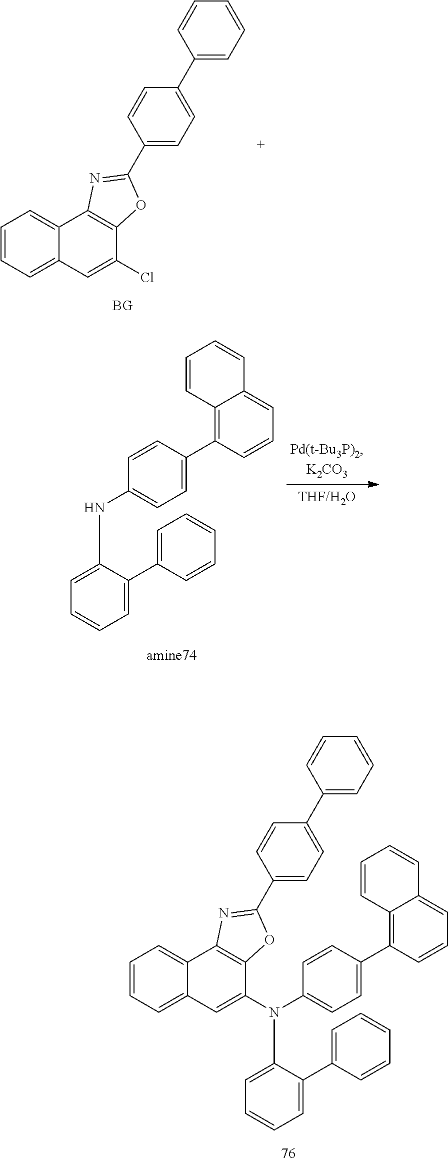 [SMILES: Clc1cc2ccccc2c2nc(-c3ccc(-c4ccccc4)cc3)oc12 ; c1ccc(-c2ccccc2Nc2ccc(-c3cccc4ccccc34)cc2)cc1 ; c1ccc(-c2ccc(-c3nc4c(o3)c(N(c3ccc(-c5cccc6ccccc56)cc3)c3ccccc3-c3ccccc3)cc3ccccc34)cc2)cc1]

Compound BG (10 g, 28.1 mmol), Compound amine74 (10.4 g, 28.1 mmol), and sodium tert-butoxide (8.9 g, 42.2 mmol) were added to xylene (200 ml) under a nitrogen atmosphere, and the mixture was stirred and refluxed. Thereafter, bis(tri-tert-butylphosphine)palladium(0) (0.1 g, 0.2 mmol) was added thereto. Upon completion of the reaction after 2 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 12.6 g of Compound 76 (Yield 65%, MS: [M+H]⁺=691).

### Example 77: Preparation of Compound 77

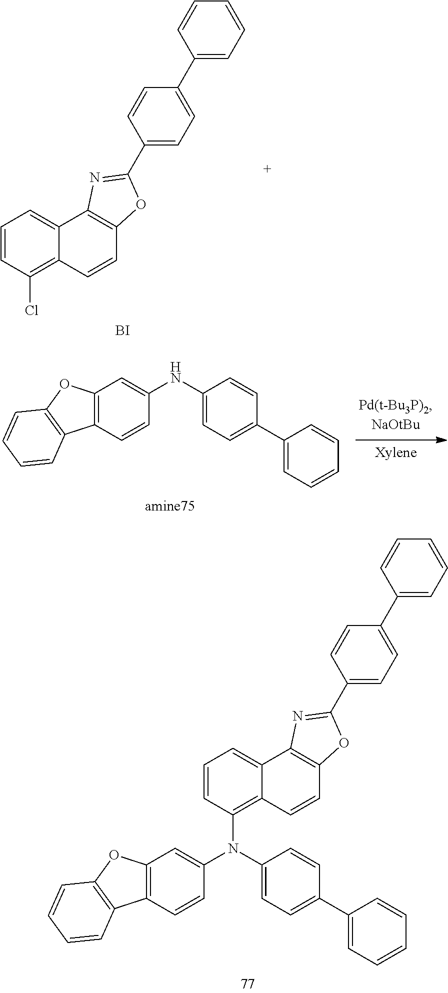 [SMILES: Clc1cccc2c1ccc1oc(-c3ccc(-c4ccccc4)cc3)nc12 ; "BI" ; c1ccc(-c2ccc(Nc3ccc4c(c3)oc3ccccc34)cc2)cc1 ; c1ccc(-c2ccc(-c3nc4c(ccc5c(N(c6ccc(-c7ccccc7)cc6)c6ccc7c(c6)oc6ccccc67)cccc54)o3)cc2)cc1]

Compound BI (10 g, 28.1 mmol), Compound amine75 (9.4 g, 28.1 mmol), and sodium tert-butoxide (8.9 g, 42.2 mmol) were added to xylene (200 ml) under a nitrogen atmosphere, and the mixture was stirred and refluxed. Thereafter, bis(tri-tert-butylphosphine)palladium(0) (0.1 g, 0.2 mmol) was added thereto. Upon completion of the reaction after 2 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 11 g of Compound 77 (Yield 60%, MS: [M+H]⁺=655).

### Example 78: Preparation of Compound 78

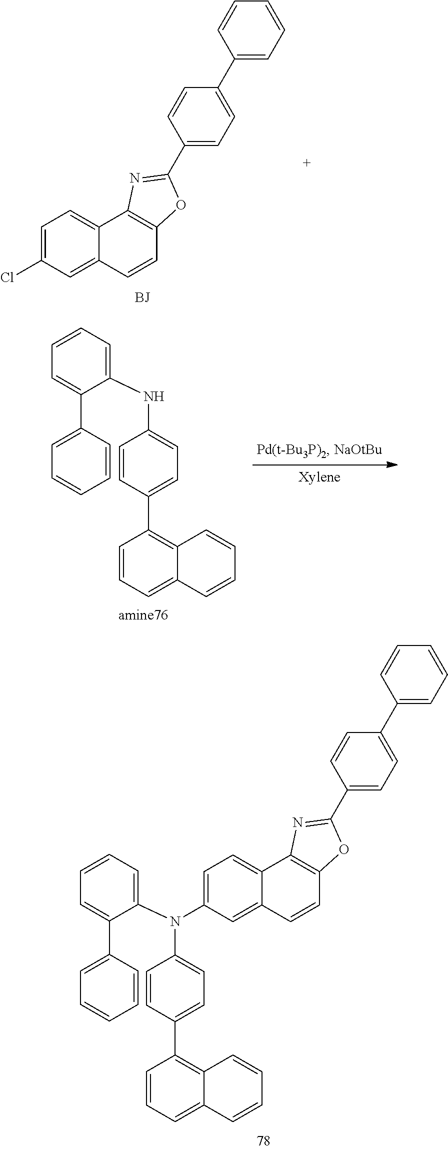

Compound BJ (10 g, 28.1 mmol), Compound amine76 (10.4 g, 28.1 mmol), and sodium tert-butoxide (8.9 g, 42.2 mmol) were added to xylene (200 ml) under a nitrogen atmosphere, and the mixture was stirred and refluxed. Thereafter, bis(tri-tert-butylphosphine)palladium(0) (0.1 g, 0.2 mmol) was added thereto. Upon completion of the reaction after 3 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 11.8 g of Compound 78 (Yield 61%, MS: [M+H]⁺=691).

### Example 79: Preparation of Compound 79

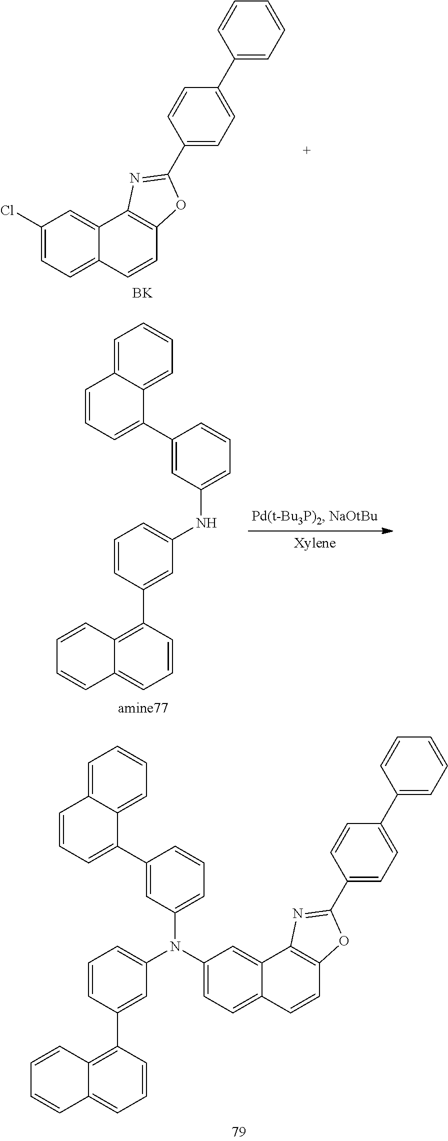

Compound BK (10 g, 28.1 mmol), Compound amine77 (11.8 g, 28.1 mmol), and sodium tert-butoxide (8.9 g, 42.2 mmol) were added to xylene (200 ml) under a nitrogen atmosphere, and the mixture was stirred and refluxed. Thereafter, bis(tri-tert-butylphosphine)palladium(0) (0.1 g, 0.2 mmol) was added thereto. Upon completion of the reaction after 2 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 13.1 g of Compound 79 (Yield 63%, MS: [M+H]⁺=741).

### Example 80: Preparation of Compound 80

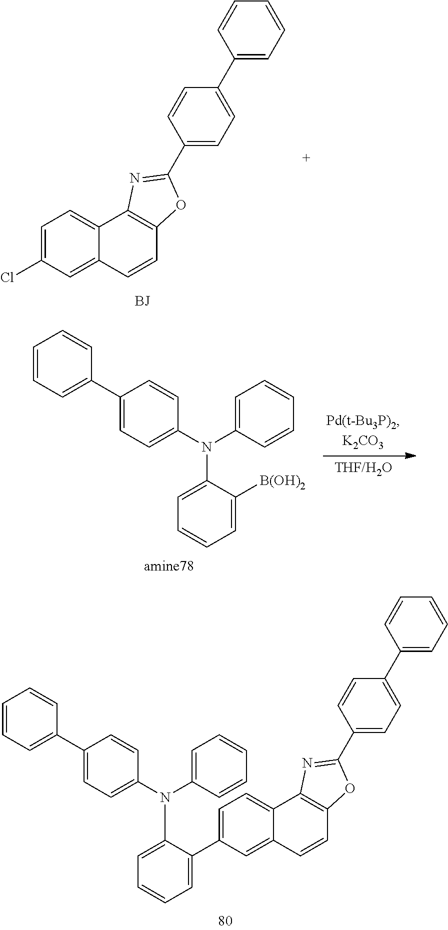 [SMILES: Clc1ccc2c(ccc3oc(-c4ccc(-c5ccccc5)cc4)nc32)c1 ; OB(O)c1ccccc1N(c1ccccc1)c1ccc(-c2ccccc2)cc1 ; c1ccc(-c2ccc(-c3nc4c(ccc5cc(-c6ccccc6N(c6ccccc6)c6ccc(-c7ccccc7)cc6)ccc54)o3)cc2)cc1]

Compound BJ (15 g, 42.2 mmol) and Compound amine78 (16.2 g, 44.3 mmol) were added to THE (300 ml) under a nitrogen atmosphere, and the mixture was stirred and refluxed. Then, potassium carbonate (17.5 g, 126.5 mmol) was dissolved in water (52 ml), and then added thereto. Thereafter, it was stirred sufficiently, followed by adding bis(tri-tert-butylphosphine)palladium(0) (0.5 g, 1.0 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 18.1 g of Compound 80 (Yield 67%, MS: [M+H]⁺=641).

### Example 81: Preparation of Compound 81

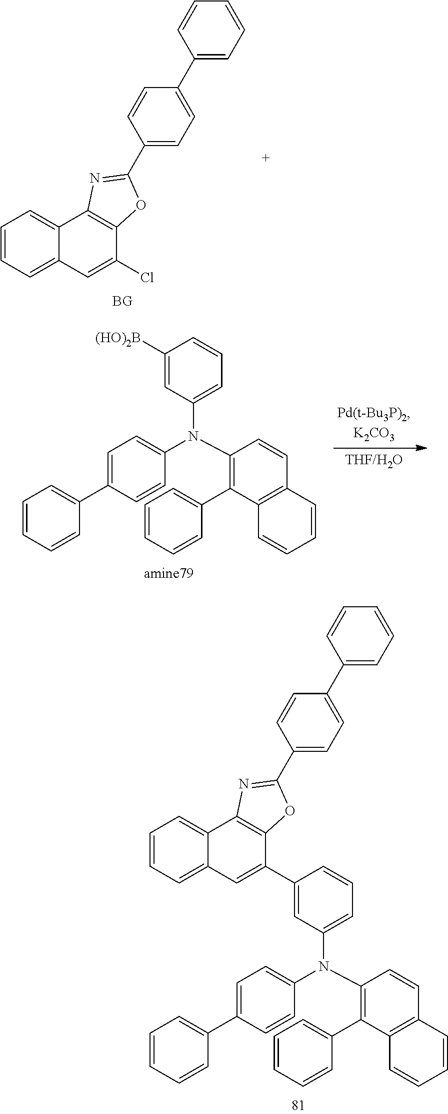 [SMILES: Clc1cc2ccccc2c2nc(-c3ccc(-c4ccccc4)cc3)oc12 ; OB(O)c1cccc(N(c2ccc(-c3ccccc3)cc2)c2ccc3ccccc3c2-c2ccccc2)c1 ; c1ccc(-c2ccc(-c3nc4c(o3)c(-c3cccc(N(c5ccc(-c6ccccc6)cc5)c5ccc6ccccc6c5-c5ccccc5)c3)cc3ccccc34)cc2)cc1]

Compound BG (15 g, 42.2 mmol) and Compound amine79 (21.8 g, 44.3 mmol) were added to THE (300 ml) under a nitrogen atmosphere, and the mixture was stirred and refluxed. Then, potassium carbonate (17.5 g, 126.5 mmol) was dissolved in water (52 ml), and then added thereto. Thereafter, it was stirred sufficiently, followed by adding bis(tri-tert-butylphosphine)palladium(0) (0.5 g, 1.0 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 19.7 g of Compound 81 (Yield 61%, MS: [M+H]⁺=767).

### Example 82: Preparation of Compound 82

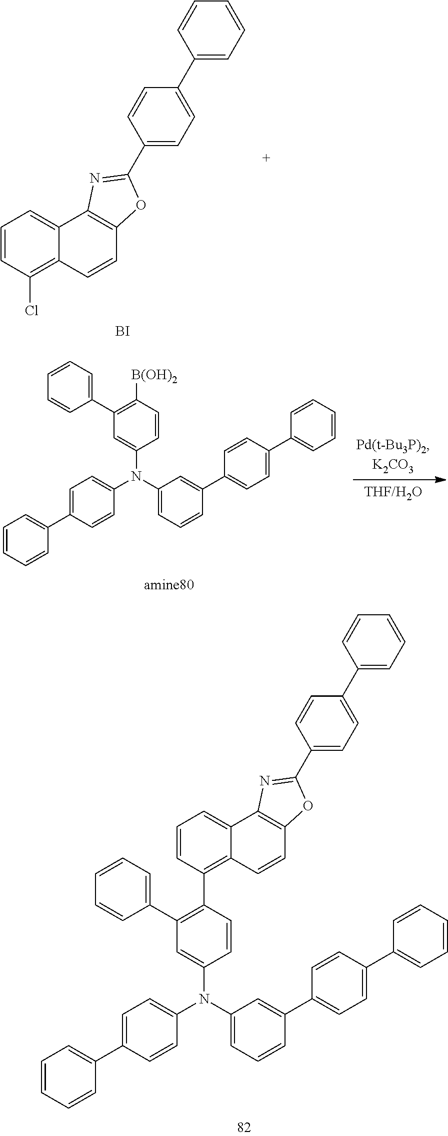 [SMILES: Clc1cccc2c1ccc1oc(-c3ccc(-c4ccccc4)cc3)nc12 ; "BI" ; OB(O)c1ccc(N(c2ccc(-c3ccccc3)cc2)c2cccc(-c3ccc(-c4ccccc4)cc3)c2)cc1-c1ccccc1 ; c1ccc(-c2ccc(-c3cccc(N(c4ccc(-c5ccccc5)cc4)c4ccc(-c5cccc6c5ccc5oc(-c7ccc(-c8ccccc8)cc7)nc56)c(-c5ccccc5)c4)c3)cc2)cc1]

Compound BI (15 g, 42.2 mmol) and Compound amine80 (26.3 g, 44.3 mmol) were added to THE (300 ml) under a nitrogen atmosphere, and the mixture was stirred and refluxed. Then, potassium carbonate (17.5 g, 126.5 mmol) was dissolved in water (52 ml), and then added thereto. Thereafter, it was stirred sufficiently, followed by adding bis(tri-tert-butylphosphine)palladium(0) (0.5 g, 1.0 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 24.9 g of Compound 82 (Yield 68%, MS: [M+H]⁺=869).

### Example 83: Preparation of Compound 83

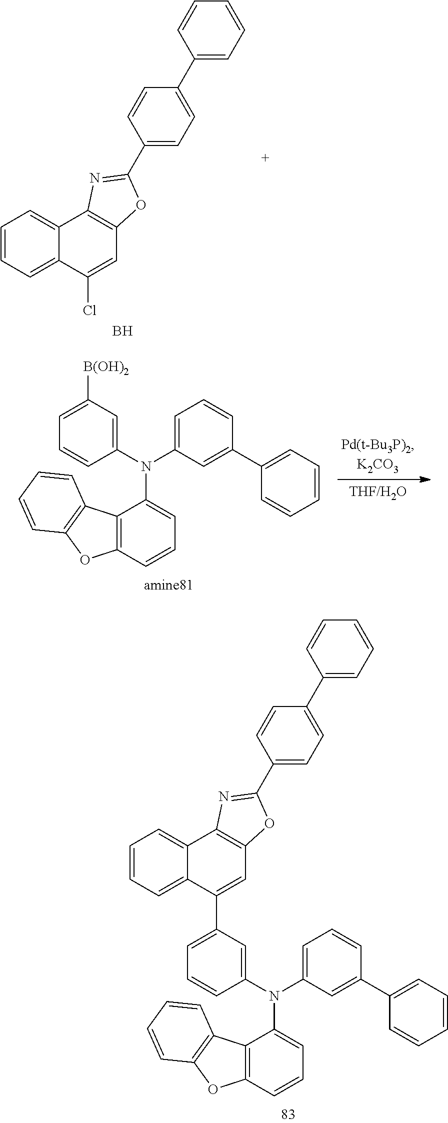

Compound BH (15 g, 42.2 mmol) and Compound amine81 (20.2 g, 44.3 mmol) were added to THE (300 ml) under a nitrogen atmosphere, and the mixture was stirred and refluxed. Then, potassium carbonate (17.5 g, 126.5 mmol) was dissolved in water (52 ml), and then added thereto. Thereafter, it was stirred sufficiently, followed by adding bis(tri-tert-butylphosphine)palladium(0) (0.5 g, 1.0 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 19.7 g of Compound 83 (Yield 64%, MS: [M+H]⁺=731).

### Example 84: Preparation of Compound 84

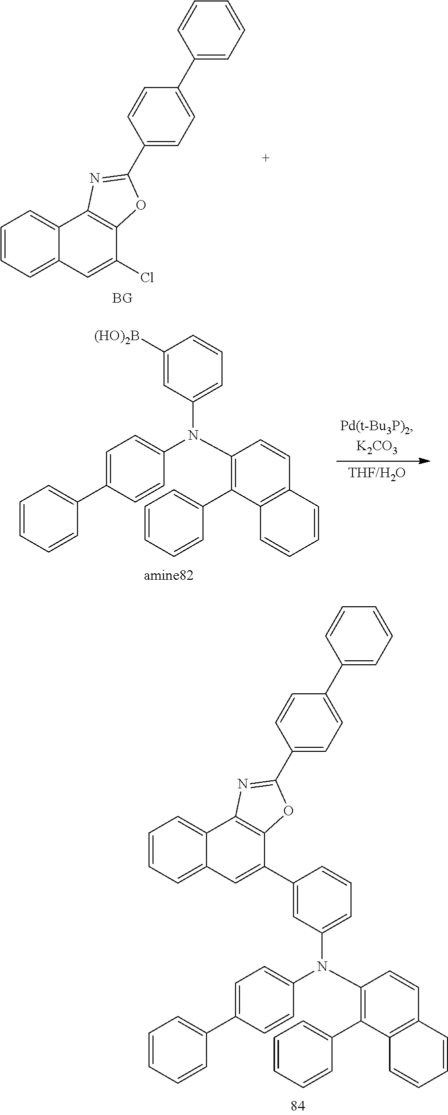

Compound BG (15 g, 42.2 mmol) and Compound amine82 (21.8 g, 44.3 mmol) were added to THE (300 ml) under a nitrogen atmosphere, and the mixture was stirred and refluxed. Then, potassium carbonate (17.5 g, 126.5 mmol) was dissolved in water (52 ml), and then added thereto. Thereafter, it was stirred sufficiently, followed by adding bis(tri-tert-butylphosphine)palladium(0) (0.5 g, 1.0 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20 g of Compound 84 (Yield 62%, MS: [M+H]⁺=767).

### Example 85: Preparation of Compound 85

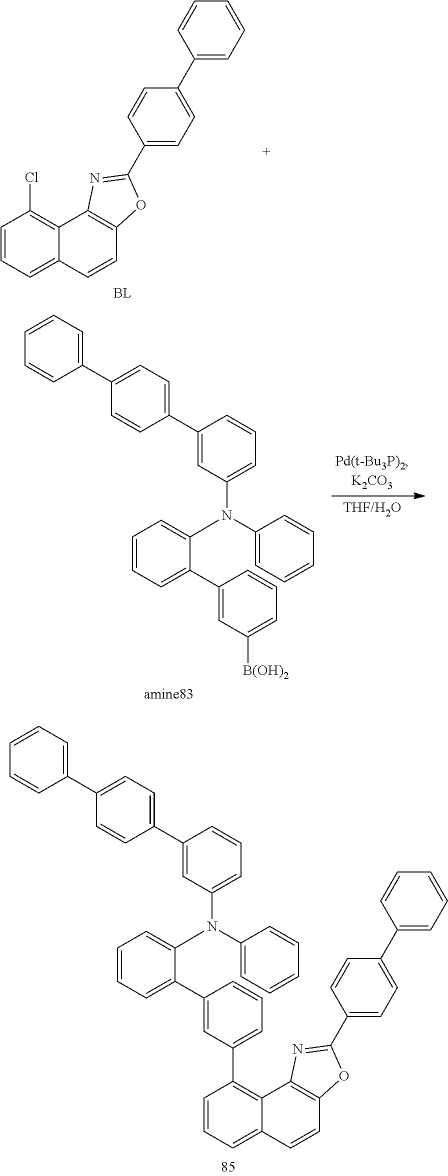

Compound BL (15 g, 42.2 mmol) and Compound amine83 (22.9 g, 44.3 mmol) were added to THE (300 ml) under a nitrogen atmosphere, and the mixture was stirred and refluxed. Then, potassium carbonate (17.5 g, 126.5 mmol) was dissolved in water (52 ml), and then added thereto. Thereafter, it was stirred sufficiently, followed by adding bis(tri-tert-butylphosphine)palladium(0) (0.5 g, 1.0 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20 g of Compound 85 (Yield 60%, MS: [M+H]⁺=793).

### Example 86: Preparation of Compound 86

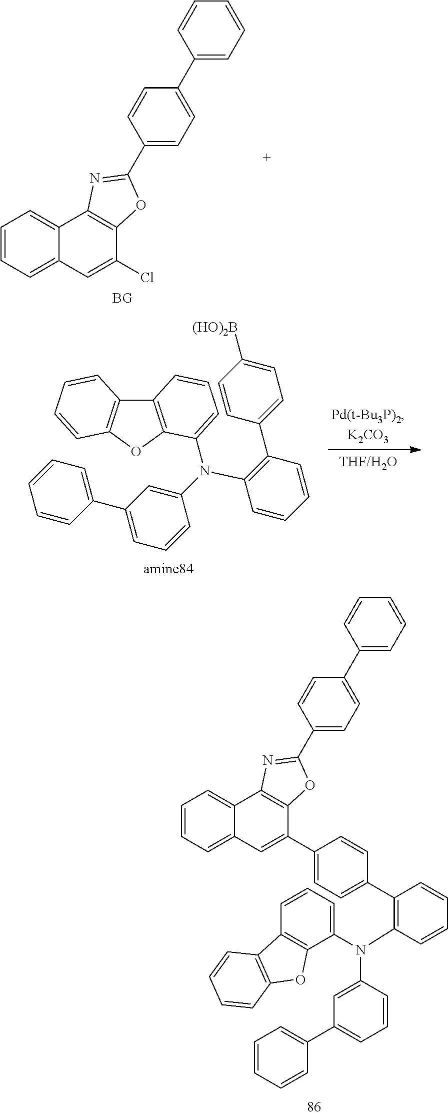

Compound BG (15 g, 42.2 mmol) and Compound amine84 (23.5 g, 44.3 mmol) were added to THE (300 ml) under a nitrogen atmosphere, and the mixture was stirred and refluxed. Then, potassium carbonate (17.5 g, 126.5 mmol) was dissolved in water (52 ml), and then added thereto. Thereafter, it was stirred sufficiently, followed by adding bis(tri-tert-butylphosphine)palladium(0) (0.5 g, 1.0 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 23.5 g of Compound 86 (Yield 69%, MS: [M+H]⁺=807).

### Example 87: Preparation of Compound 87

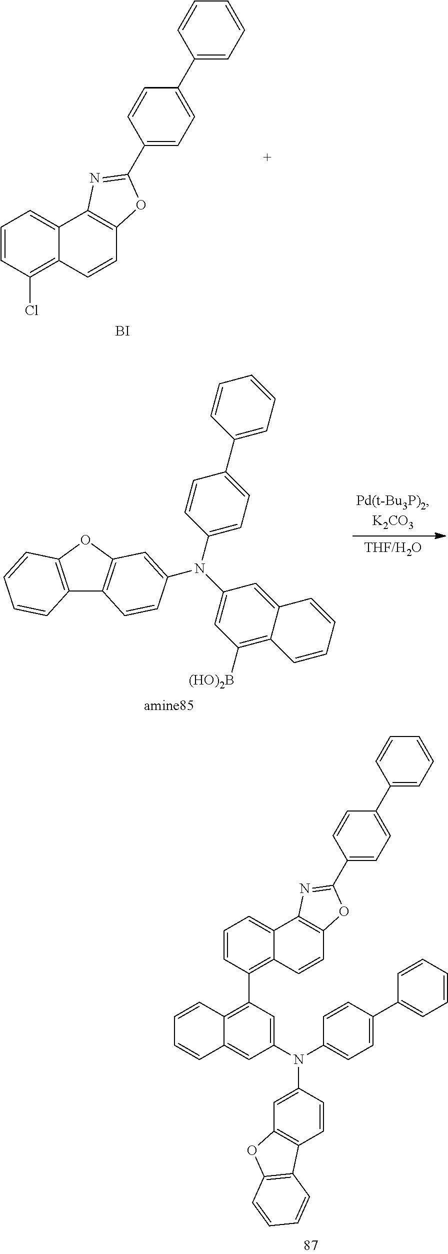 [SMILES: Clc1cccc2c1ccc1oc(-c3ccc(-c4ccccc4)cc3)nc12 ; "BI" ; OB(O)c1cc(N(c2ccc(-c3ccccc3)cc2)c2ccc3c(c2)oc2ccccc23)cc2ccccc12 ; c1ccc(-c2ccc(-c3nc4c(ccc5c(-c6cc(N(c7ccc(-c8ccccc8)cc7)c7ccc8c(c7)oc7ccccc78)cc7ccccc67)cccc54)o3)cc2)cc1]

Compound BI (15 g, 42.2 mmol) and Compound amine85 (22.4 g, 44.3 mmol) were added to THE (300 ml) under a nitrogen atmosphere, and the mixture was stirred and refluxed. Then, potassium carbonate (17.5 g, 126.5 mmol) was dissolved in water (52 ml), and then added thereto. Thereafter, it was stirred sufficiently, followed by adding bis(tri-tert-butylphosphine)palladium(0) (0.5 g, 1.0 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 22.7 g of Compound 87 (Yield 69%, MS: [M+H]⁺=781).

### Example 88: Preparation of Compound 88

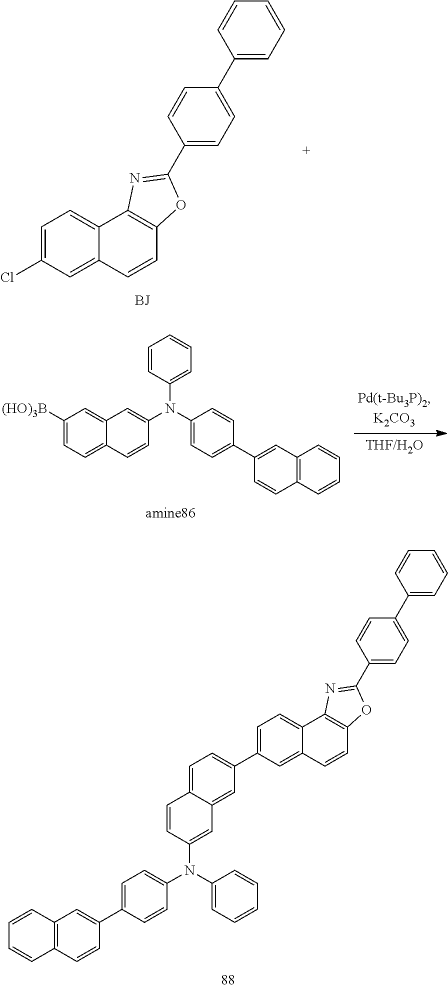

Compound BJ (15 g, 42.2 mmol) and Compound amine86 (20.6 g, 44.3 mmol) were added to THE (300 ml) under a nitrogen atmosphere, and the mixture was stirred and refluxed. Then, potassium carbonate (17.5 g, 126.5 mmol) was dissolved in water (52 ml), and then added thereto. Thereafter, it was stirred sufficiently, followed by adding bis(tri-tert-butylphosphine)palladium(0) (0.5 g, 1.0 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20.6 g of Compound 88 (Yield 66%, MS: [M+H]⁺=741).

### Example 89: Preparation of Compound 89

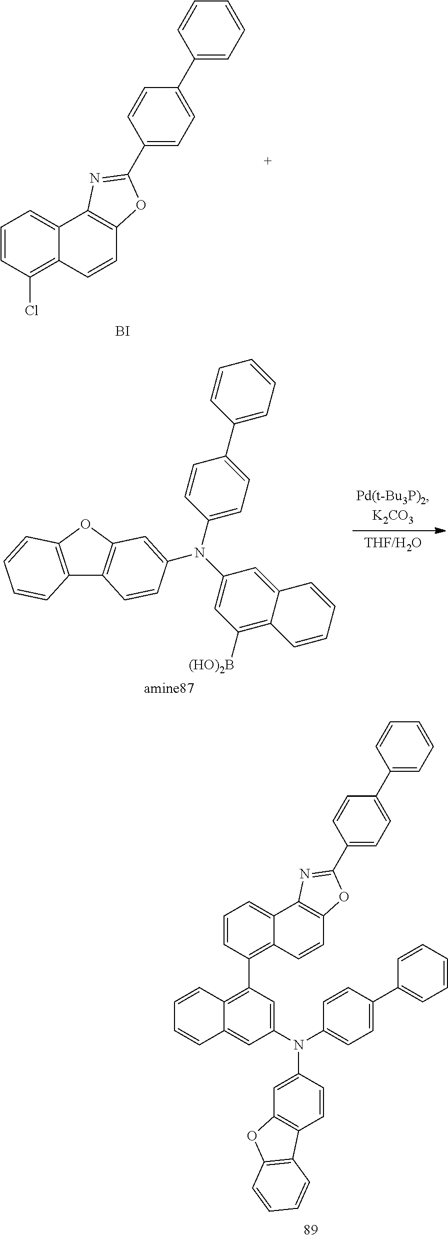 [SMILES: Clc1cccc2c1ccc1oc(-c3ccc(-c4ccccc4)cc3)nc12 ; "BI" ; OB(O)c1cc(N(c2ccc(-c3ccccc3)cc2)c2ccc3c(c2)oc2ccccc23)cc2ccccc12 ; c1ccc(-c2ccc(-c3nc4c(ccc5c(-c6cc(N(c7ccc(-c8ccccc8)cc7)c7ccc8c(c7)oc7ccccc78)cc7ccccc67)cccc54)o3)cc2)cc1]

Compound BI (15 g, 42.2 mmol) and Compound amine87 (22.4 g, 44.3 mmol) were added to THE (300 ml) under a nitrogen atmosphere, and the mixture was stirred and refluxed. Then, potassium carbonate (17.5 g, 126.5 mmol) was dissolved in water (52 ml), and then added thereto. Thereafter, it was stirred sufficiently, followed by adding bis(tri-tert-butylphosphine)palladium(0) (0.5 g, 1.0 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20.4 g of Compound 89 (Yield 62%, MS: [M+H]⁺=781).

### Example 90: Preparation of Compound 90

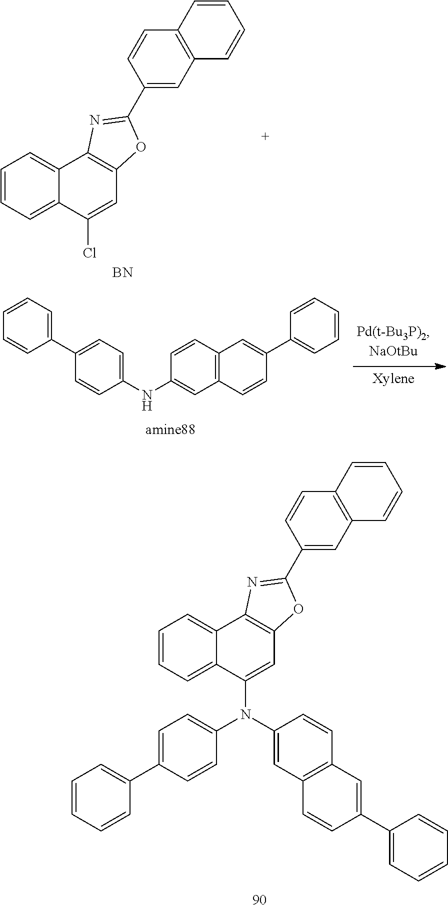

Compound BN (10 g, 30.3 mmol), Compound amine88 (11.3 g, 30.3 mmol), and sodium tert-butoxide (9.7 g, 45.5 mmol) were added to xylene (200 ml) under a nitrogen atmosphere, and the mixture was stirred and refluxed. Thereafter, bis(tri-tert-butylphosphine)palladium(0) (0.2 g, 0.4 mmol) was added thereto. Upon completion of the reaction after 3 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 13.3 g of Compound 90 (Yield 66%, MS: [M+H]⁺=665).

### Example 91: Preparation of Compound 91

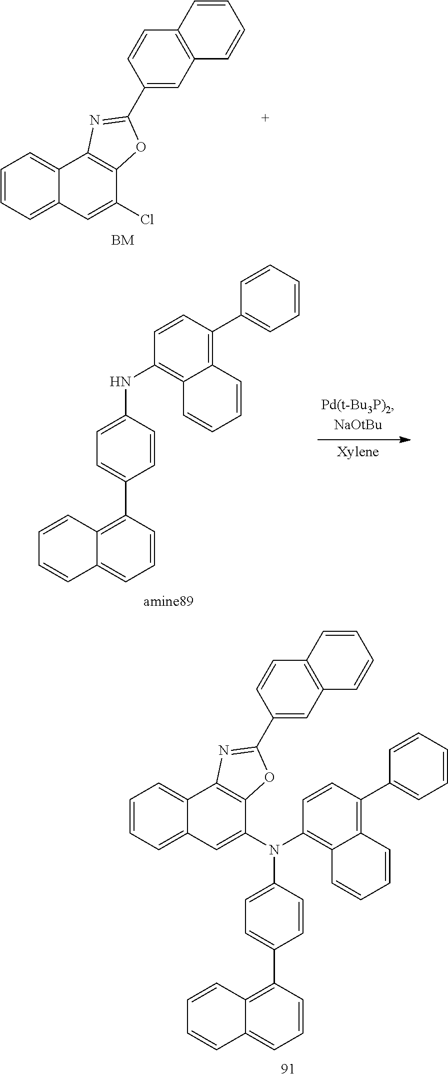

Compound BM (10 g, 30.3 mmol), Compound amine89 (12.8 g, 30.3 mmol), and sodium tert-butoxide (9.7 g, 45.5 mmol) were added to xylene (200 ml) under a nitrogen atmosphere, and the mixture was stirred and refluxed. Thereafter, bis(tri-tert-butylphosphine)palladium(0) (0.2 g, 0.4 mmol) was added thereto. Upon completion of the reaction after 2 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 13.4 g of Compound 91 (Yield 62%, MS: [M+H]⁺=715).

### Example 92: Preparation of Compound 92

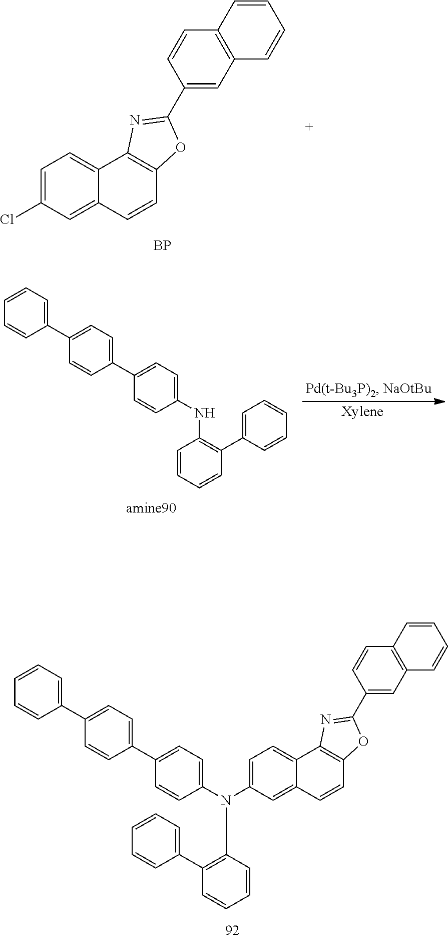

Compound BP (10 g, 30.3 mmol), Compound amine90 (12.1 g, 30.3 mmol), and sodium tert-butoxide (9.7 g, 45.5 mmol) were added to xylene (200 ml) under a nitrogen atmosphere, and the mixture was stirred and refluxed. Thereafter, bis(tri-tert-butylphosphine)palladium(0) (0.2 g, 0.4 mmol) was added thereto. Upon completion of the reaction after 3 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 13.8 g of Compound 92 (Yield 66%, MS: [M+H]⁺=691).

### Example 93: Preparation of Compound 93

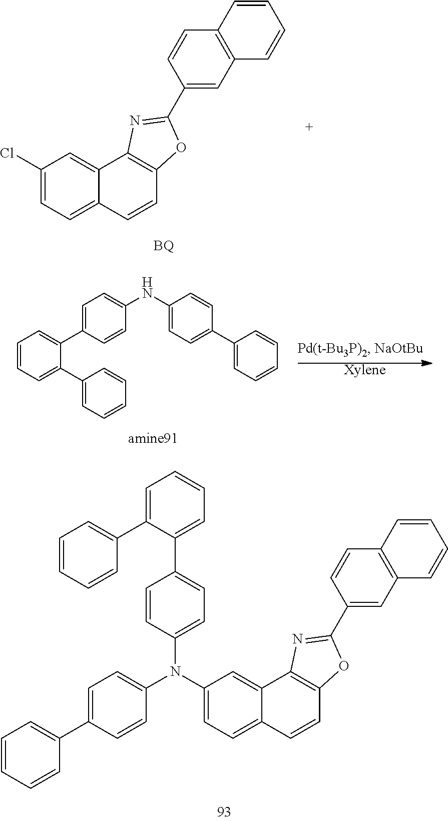

Compound BQ (10 g, 30.3 mmol), Compound amine91 (12.1 g, 30.3 mmol), and sodium tert-butoxide (9.7 g, 45.5 mmol) were added to xylene (200 ml) under a nitrogen atmosphere, and the mixture was stirred and refluxed. Thereafter, bis(tri-tert-butylphosphine)palladium(0) (0.2 g, 0.4 mmol) was added thereto. Upon completion of the reaction after 3 hours, the solvent was removed by cooling to room temperature and reducing the pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 13.4 g of Compound 93 (Yield 64%, MS: [M+H]⁺=691).

### Example 94: Preparation of Compound 94

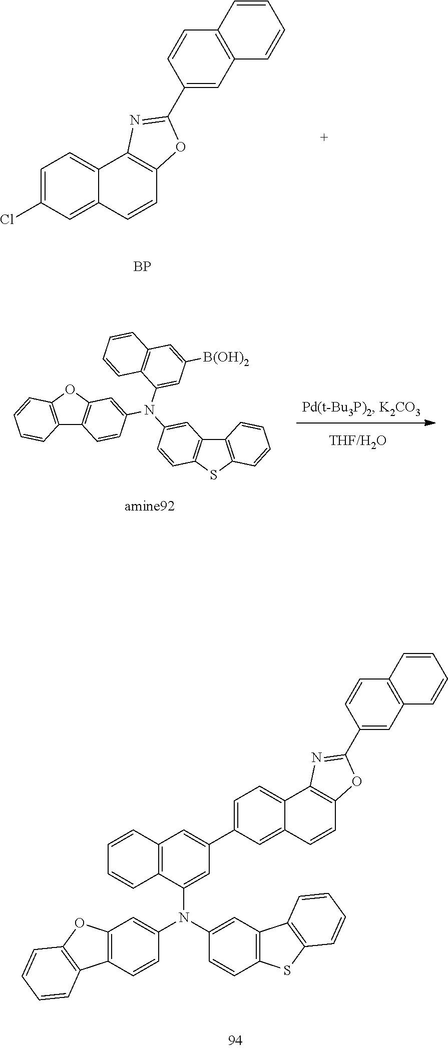 [SMILES: Clc1ccc2c(ccc3oc(-c4ccc5ccccc5c4)nc32)c1 ; "BP" ; OB(O)c1cc(N(c2ccc3c(c2)oc2ccccc23)c2ccc3sc4ccccc4c3c2)c2ccccc2c1 ; c1ccc2cc(-c3nc4c(ccc5cc(-c6cc(N(c7ccc8c(c7)oc7ccccc78)c7ccc8sc9ccccc9c8c7)c7ccccc7c6)ccc54)o3)ccc2c1]

Compound BP (15 g, 45.5 mmol) and Compound amine92 (25.6 g, 47.8 mmol) were added to THE (300 ml) under a nitrogen atmosphere, and the mixture was stirred and refluxed. Then, potassium carbonate (18.9 g, 136.5 mmol) was dissolved in water (57 ml), and then added thereto. Thereafter, it was stirred sufficiently, followed by adding bis(tri-tert-butylphosphine)palladium(0) (0.5 g, 1.0 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 21.8 g of Compound 94 (Yield 61%, MS: [M+H]⁺=785).

### Example 95: Preparation of Compound 95

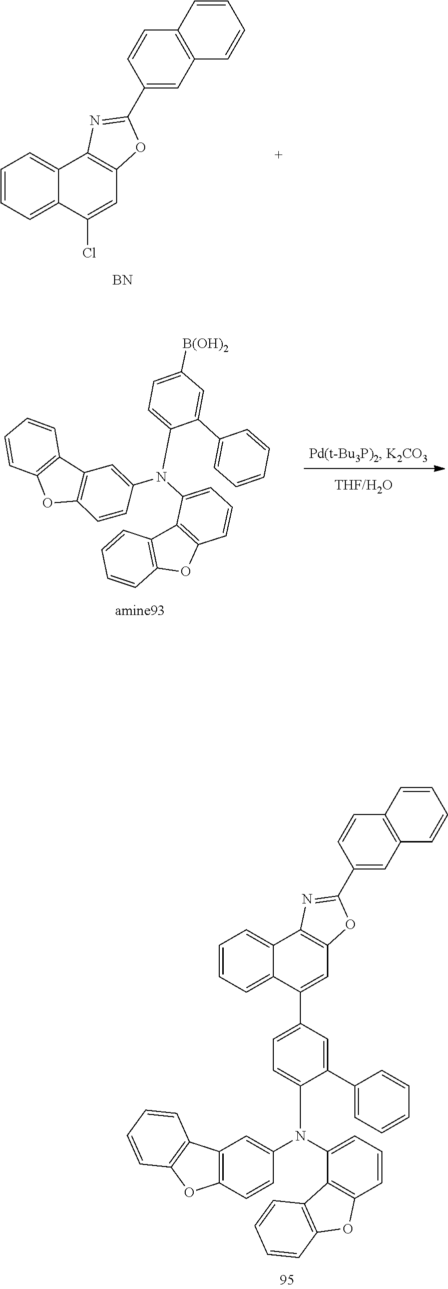

Compound BN (15 g, 45.5 mmol) and Compound amine93 (26 g, 47.8 mmol) were added to THE (300 ml) under a nitrogen atmosphere, and the mixture was stirred and refluxed. Then, potassium carbonate (18.9 g, 136.5 mmol) was dissolved in water (57 ml), and then added thereto. Thereafter, it was stirred sufficiently, followed by adding bis(tri-tert-butylphosphine)palladium(0) (0.5 g, 1.0 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 24.6 g of Compound 95 (Yield 68%, MS: [M+H]⁺=795).

### Example 96: Preparation of Compound 96

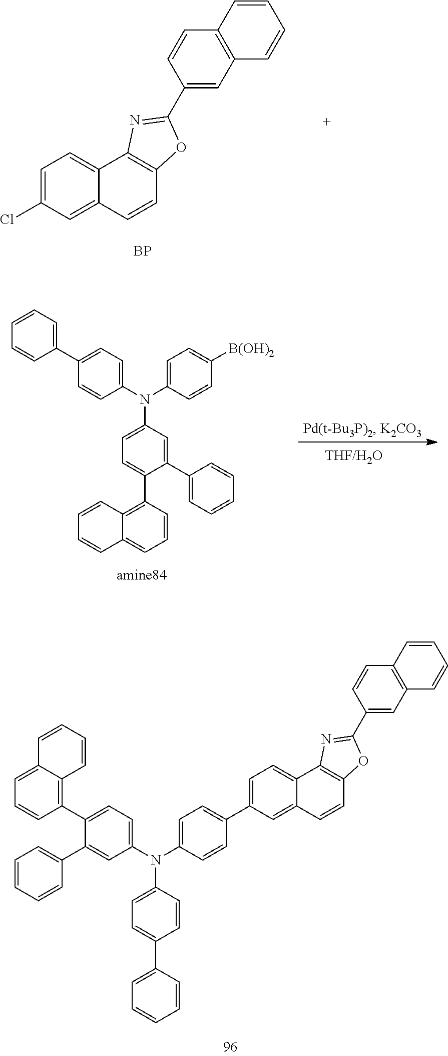

Compound BP (15 g, 45.5 mmol) and Compound amine94 (27.1 g, 47.8 mmol) were added to THE (300 ml) under a nitrogen atmosphere, and the mixture was stirred and refluxed. Then, potassium carbonate (18.9 g, 136.5 mmol) was dissolved in water (57 ml), and then added thereto. Thereafter, it was stirred sufficiently, followed by adding bis(tri-tert-butylphosphine)palladium(0) (0.5 g, 1.0 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 25.6 g of Compound 96 (Yield 69%, MS: [M+H]⁺=817).

### Example 97: Preparation of Compound 97

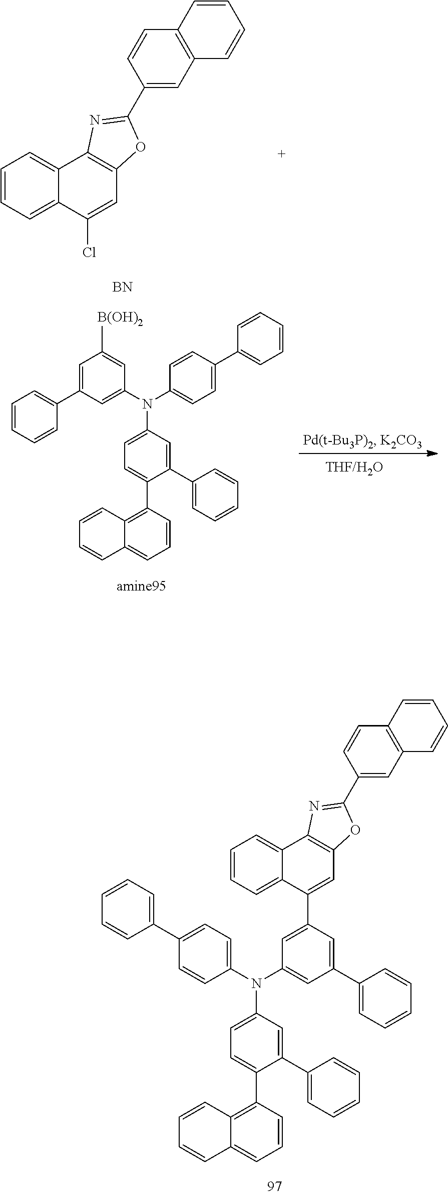

Compound BN (15 g, 45.5 mmol) and Compound amine95 (30.7 g, 47.8 mmol) were added to THF (300 ml) under a nitrogen atmosphere, and the mixture was stirred and refluxed. Then, potassium carbonate (18.9 g, 136.5 mmol) was dissolved in water (57 ml), and then added thereto. Thereafter, it was stirred sufficiently, followed by adding bis(tri-tert-butylphosphine)palladium(0) (0.5 g, 1.0 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 25.2 g of Compound 97 (Yield 62%, MS: [M+H]⁺=893).

### Example 98: Preparation of Compound 98

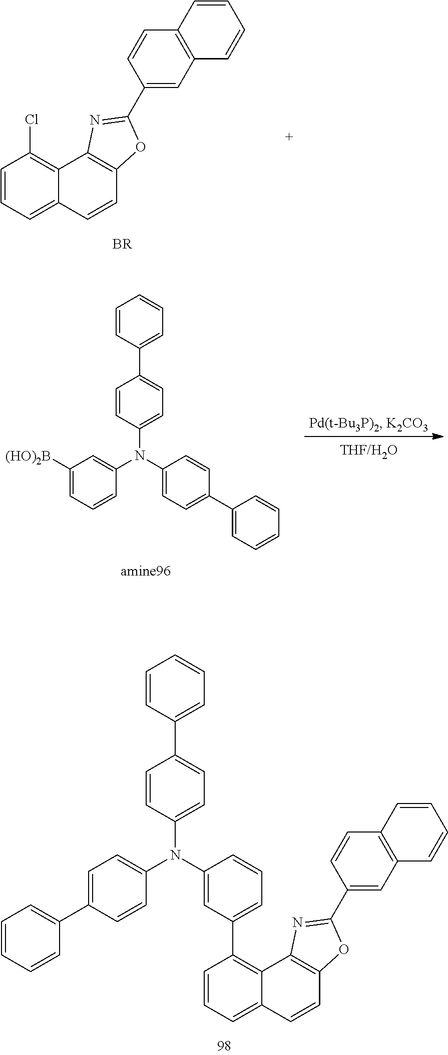 [SMILES: Clc1cccc2ccc3oc(-c4ccc5ccccc5c4)nc3c12 ; OB(O)c1cccc(N(c2ccc(-c3ccccc3)cc2)c2ccc(-c3ccccc3)cc2)c1 ; c1ccc(-c2ccc(N(c3ccc(-c4ccccc4)cc3)c3cccc(-c4cccc5ccc6oc(-c7ccc8ccccc8c7)nc6c45)c3)cc2)cc1]

Compound BR (15 g, 45.5 mmol) and Compound amine96 (21.1 g, 47.8 mmol) were added to THE (300 ml) under a nitrogen atmosphere, and the mixture was stirred and refluxed. Then, potassium carbonate (18.9 g, 136.5 mmol) was dissolved in water (57 ml), and then added thereto. Thereafter, it was stirred sufficiently, followed by adding bis(tri-tert-butylphosphine)palladium(0) (0.5 g, 1.0 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20.1 g of Compound 98 (Yield 64%, MS: [M+H]⁺=691).

### Example 99: Preparation of Compound 99

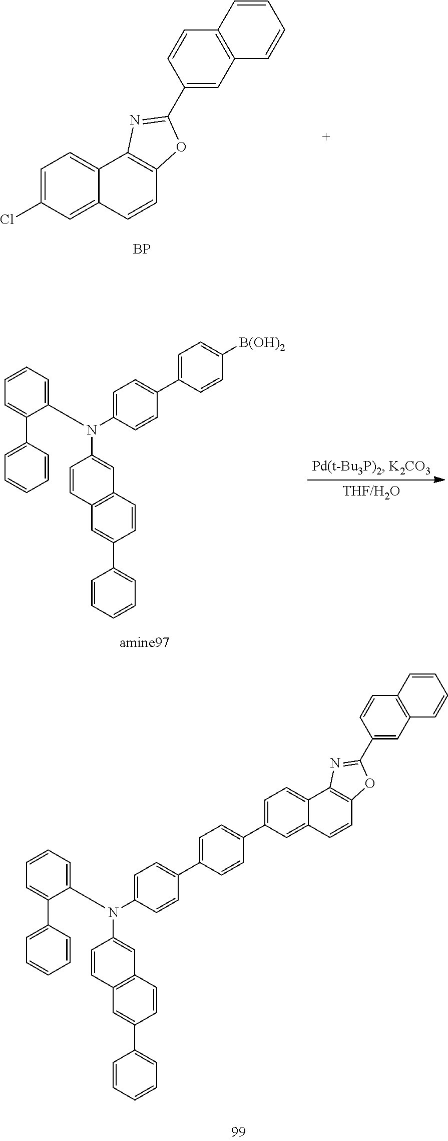

Compound BP (15 g, 45.5 mmol) and Compound amine97 (27.1 g, 47.8 mmol) were added to THE (300 ml) under a nitrogen atmosphere, and the mixture was stirred and refluxed. Then, potassium carbonate (18.9 g, 136.5 mmol) was dissolved in water (57 ml), and then added thereto. Thereafter, it was stirred sufficiently, followed by adding bis(tri-tert-butylphosphine)palladium(0) (0.5 g, 1.0 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 23 g of Compound 99 (Yield 62%, MS: [M+H]⁺=817).

### Example 100: Preparation of Compound 100

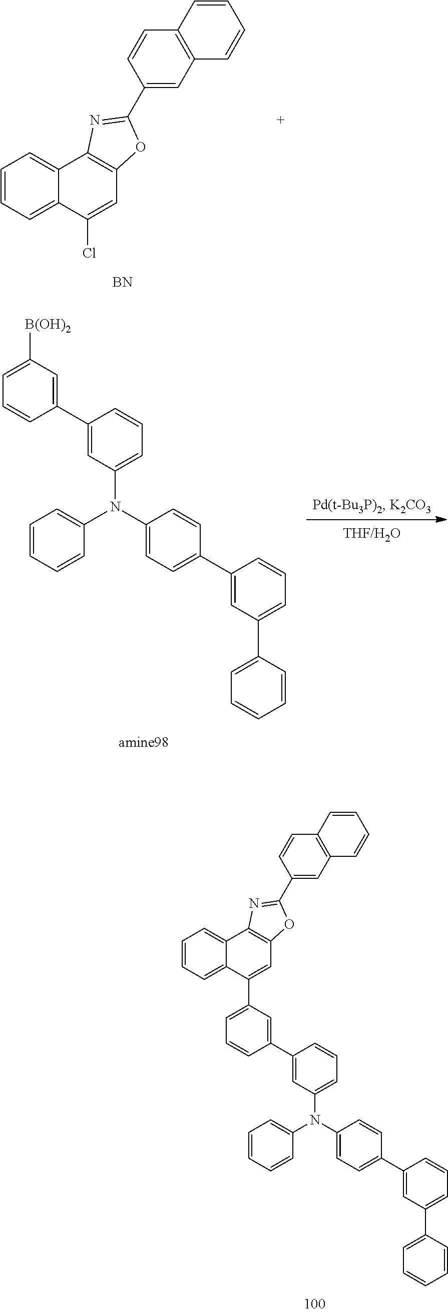

Compound BB (15 g, 45.5 mmol) and Compound amine98 (24.7 g, 47.8 mmol) were added to THE (300 ml) under a nitrogen atmosphere, and the mixture was stirred and refluxed. Then, potassium carbonate (18.9 g, 136.5 mmol) was dissolved in water (57 ml), and then added thereto. Thereafter, it was stirred sufficiently, followed by adding bis(tri-tert-butylphosphine)palladium(0) (0.5 g, 1.0 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 22.3 g of Compound 100 (Yield 64%, MS: [M+H]⁺=767).

### Example 101: Preparation of Compound 101

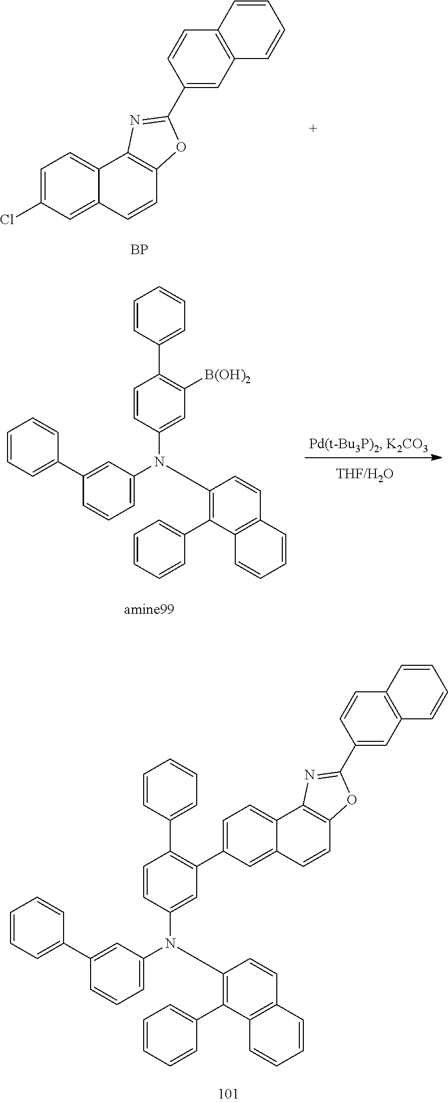

Compound BP (15 g, 45.5 mmol) and Compound amine99 (27.1 g, 47.8 mmol) were added to THE (300 ml) under a nitrogen atmosphere, and the mixture was stirred and refluxed. Then, potassium carbonate (18.9 g, 136.5 mmol) was dissolved in water (57 ml), and then added thereto. Thereafter, it was stirred sufficiently, followed by adding bis(tri-tert-butylphosphine)palladium(0) (0.5 g, 1.0 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 22.3 g of Compound 101 (Yield 60%, MS: [M+H]⁺=817).

### Example 102: Preparation of Compound 102

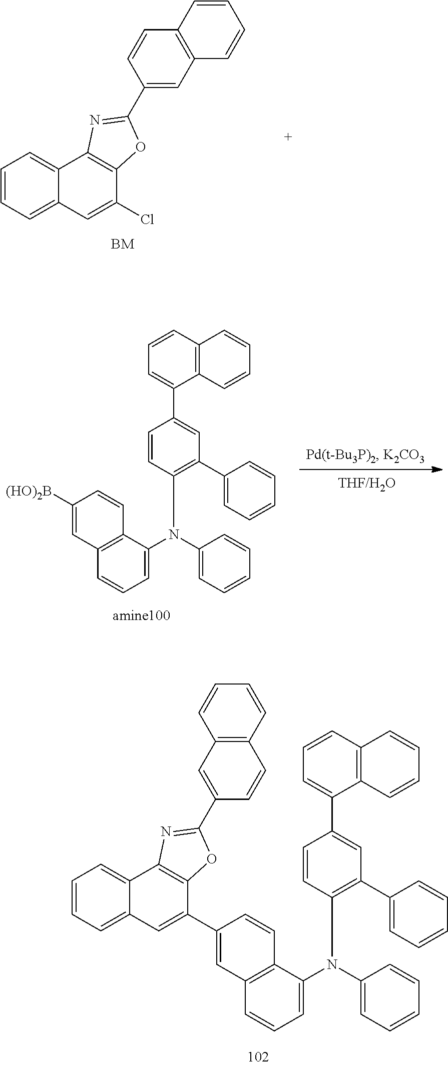 [SMILES: Clc1cc2ccccc2c2nc(-c3ccc4ccccc4c3)oc12 ; OB(O)c1ccc2c(N(Cc3ccc(-c4cccc5ccccc45)cc3-c3ccccc3)c3ccccc3)cccc2c1 ; c1ccc(-c2cc(-c3cccc4ccccc34)ccc2CN(c2ccccc2)c2cccc3cc(-c4cc5ccccc5c5nc(-c6ccc7ccccc7c6)oc45)ccc23)cc1]

Compound BM (15 g, 45.5 mmol) and Compound amine100 (25.9 g, 47.8 mmol) were added to THE (300 ml) under a nitrogen atmosphere, and the mixture was stirred and refluxed. Then, potassium carbonate (18.9 g, 136.5 mmol) was dissolved in water (57 ml), and then added thereto. Thereafter, it was stirred sufficiently, followed by adding bis(tri-tert-butylphosphine)palladium(0) (0.5 g, 1.0 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 21.6 g of Compound 102 (Yield 60%, MS: [M+H]⁺=791).

### Example 103: Preparation of Compound 103

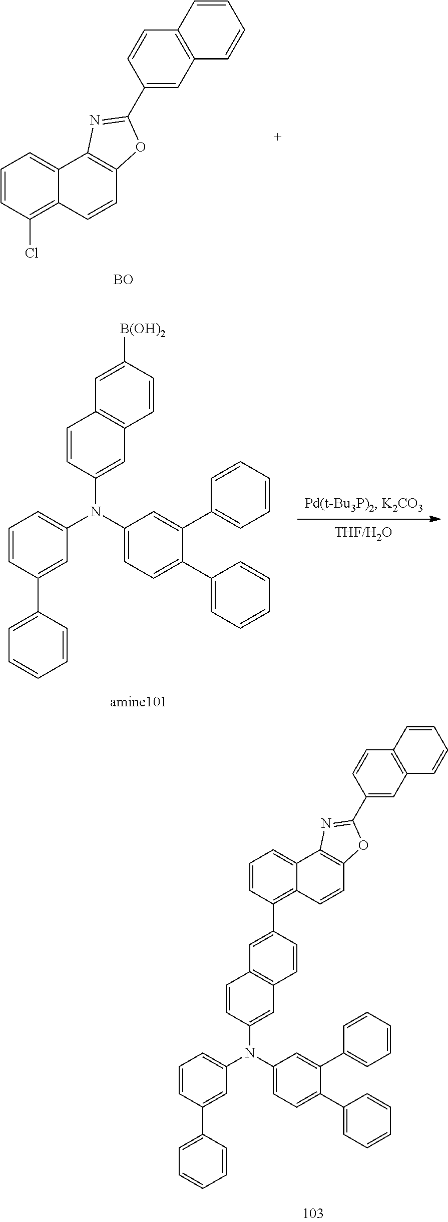 [SMILES: Clc1cccc2c1ccc1oc(-c3ccc4ccccc4c3)nc12 ; "BO" ; OB(O)c1ccc2cc(N(c3cccc(-c4ccccc4)c3)c3ccc(-c4ccccc4)c(-c4ccccc4)c3)ccc2c1 ; c1ccc(-c2cccc(N(c3ccc(-c4ccccc4)c(-c4ccccc4)c3)c3ccc4cc(-c5cccc6c5ccc5oc(-c7ccc8ccccc8c7)nc56)ccc4c3)c2)cc1]

Compound BO (15 g, 45.5 mmol) and Compound amine101 (27.1 g, 47.8 mmol) were added to THE (300 ml) under a nitrogen atmosphere, and the mixture was stirred and refluxed. Then, potassium carbonate (18.9 g, 136.5 mmol) was dissolved in water (57 ml), and then added thereto. Thereafter, it was stirred sufficiently, followed by adding bis(tri-tert-butylphosphine)palladium(0) (0.5 g, 1.0 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 26 g of Compound 103 (Yield 70%, MS: [M+H]⁺=817).

### Example 104: Preparation of Compound 104

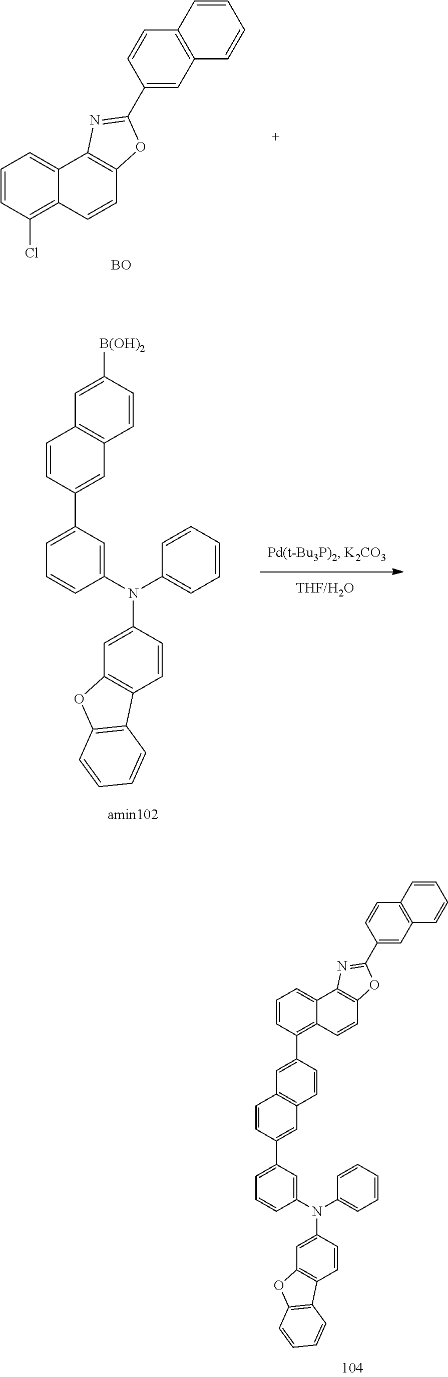 [SMILES: Clc1cccc2c1ccc1oc(-c3ccc4ccccc4c3)nc12 ; "BO" ; OB(O)c1ccc2cc(-c3cccc(N(c4ccccc4)c4ccc5c(c4)oc4ccccc45)c3)ccc2c1 ; c1ccc(N(c2cccc(-c3ccc4cc(-c5cccc6c5ccc5oc(-c7ccc8ccccc8c7)nc56)ccc4c3)c2)c2ccc3c(c2)oc2ccccc23)cc1]

Compound BO (15 g, 45.5 mmol) and Compound amine102 (24.1 g, 47.8 mmol) were added to THE (300 ml) under a nitrogen atmosphere, and the mixture was stirred and refluxed. Then, potassium carbonate (18.9 g, 136.5 mmol) was dissolved in water (57 ml), and then added thereto. Thereafter, it was stirred sufficiently, followed by adding bis(tri-tert-butylphosphine)palladium(0) (0.5 g, 1.0 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 22.6 g of Compound 104 (Yield 63%, MS: [M+H]⁺=791).

### Example 105: Preparation of Compound 105

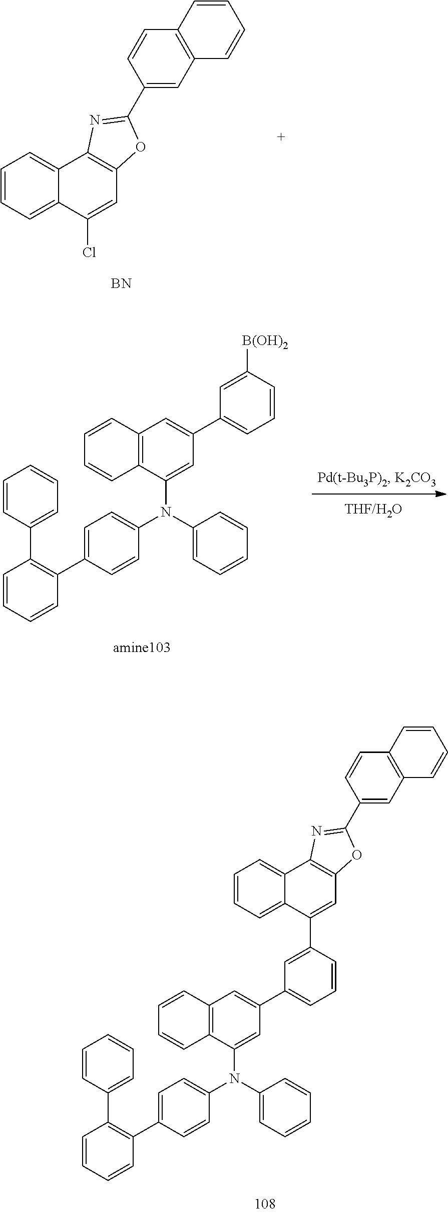

Compound BN (15 g, 45.5 mmol) and Compound amine103 (27.1 g, 47.8 mmol) were added to THE (300 ml) under a nitrogen atmosphere, and the mixture was stirred and refluxed. Then, potassium carbonate (18.9 g, 136.5 mmol) was dissolved in water (57 ml), and then added thereto. Thereafter, it was stirred sufficiently, followed by adding bis(tri-tert-butylphosphine)palladium(0) (0.5 g, 1.0 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 23.7 g of Compound 105 (Yield 69%, MS: [M+H]⁺=755).

### EXPERIMENTAL EXAMPLES

### Experimental Example 1

A glass substrate on which ITO (Indium Tin Oxide) was coated as a thin film to a thickness of 1,000 Å was put into distilled water in which a detergent was dissolved, and ultrasonically cleaned. At this time, a product manufactured by Fischer Co. was used as the detergent, and distilled water filtered twice using a filter manufactured by Millipore Co. was used as the distilled water. After the ITO was cleaned for 30 minutes, ultrasonic cleaning was repeated twice using distilled water for 10 minutes. After the cleaning with distilled water was completed, the substrate was ultrasonically cleaned with solvents of isopropyl alcohol, acetone, and methanol, dried, and then transferred to a plasma cleaner. In addition, the substrate was cleaned for 5 minutes using oxygen plasma and then transferred to a vacuum depositor.

On the prepared ITO transparent electrode, the following Compound HI-1 was formed to a thickness of 1150 Å while the following Compound A-1 was p-doped at a concentration of 1.5% to form a hole injection layer. On the hole injection layer, the following Compound HT-1 was vacuum-deposited to form a hole transport layer having a thickness of 800 Å. Then, on the hole transport layer, the Compound 1 prepared above was vacuum-deposited to form an electron blocking layer having a thickness of 150 Å. On the electron blocking layer, the following Compound RH-1 as a host and the following Compound Dp-7 as a dopant were vacuum-deposited at a weight ratio of 98:2 to form a red light emitting layer having a thickness of 400 Å. On the light emitting layer, the following Compound HB-1 was vacuum-deposited to form a hole blocking layer having a thickness of 30 Å. On the hole blocking layer, the following Compound ET-1 and the following Compound LiQ were vacuum-deposited at a weight ratio of 2:1 to form an electron injection and transport layer having a thickness of 300 Å. On the electron injection and transport layer, lithium fluoride (LiF) and aluminum were sequentially deposited to a thickness of 12 Å and 1,000 Å, respectively to form a cathode.

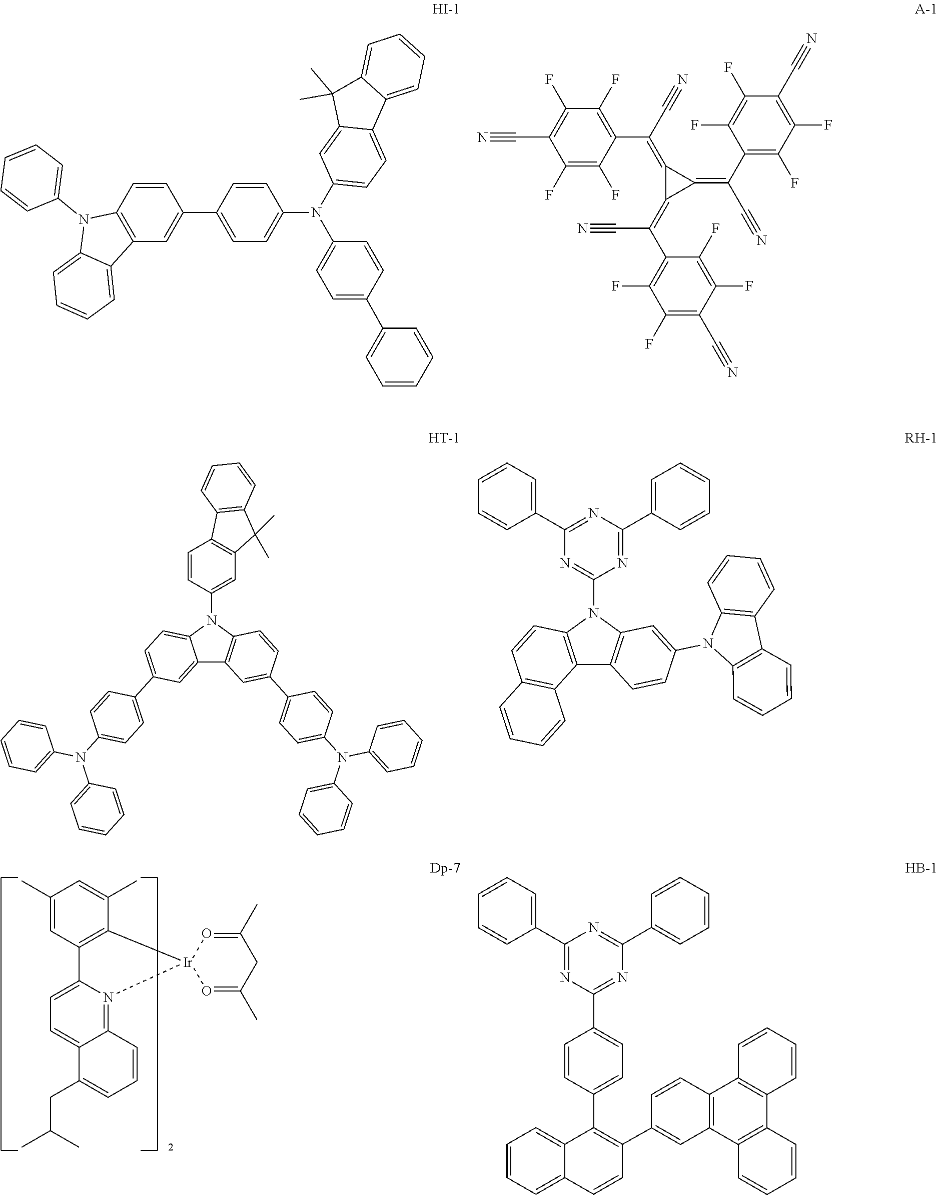

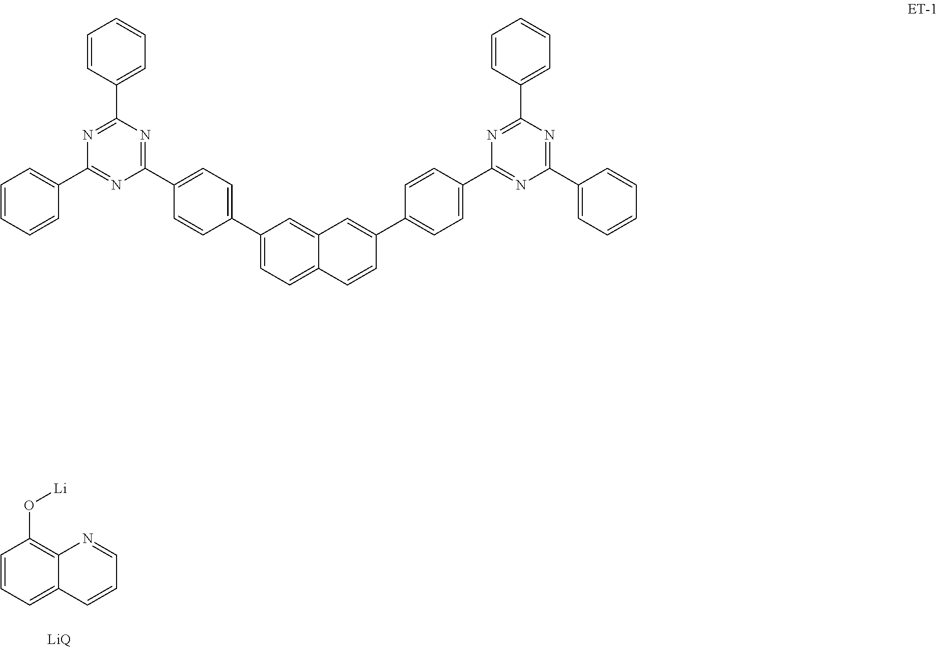

In the above process, the deposition rate of the organic material was maintained at 0.4 to 0.7 Å/sec, the deposition rate of lithium fluoride of the cathode was maintained at 0.3 Å/sec, and the deposition rate of aluminum was maintained at 2 Å/sec. In addition, the degree of vacuum during the deposition was maintained at 2×10⁻⁷ to 5×10⁻⁶ torr, thereby manufacturing an organic light emitting device.

### Experimental Examples 2 to 105

An organic light emitting device was manufactured in the same manner as in Experimental Example 1, except that the compound shown in Tables 1 to 5 was used instead of Compound 1.

### Comparative Experimental Examples 1 to 16

An organic light emitting device was manufactured in the same manner as in Experimental Example 1, except that the compound shown in Table 6 was used instead of Compound 1. At this time, Compounds C-1 to C-16 listed in Table 6 are as follows.

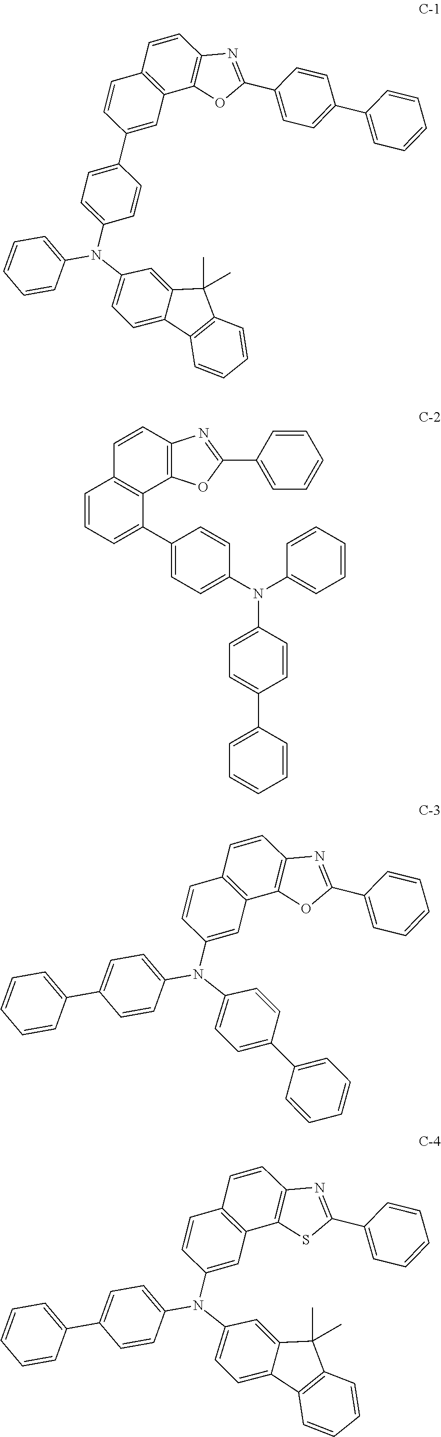

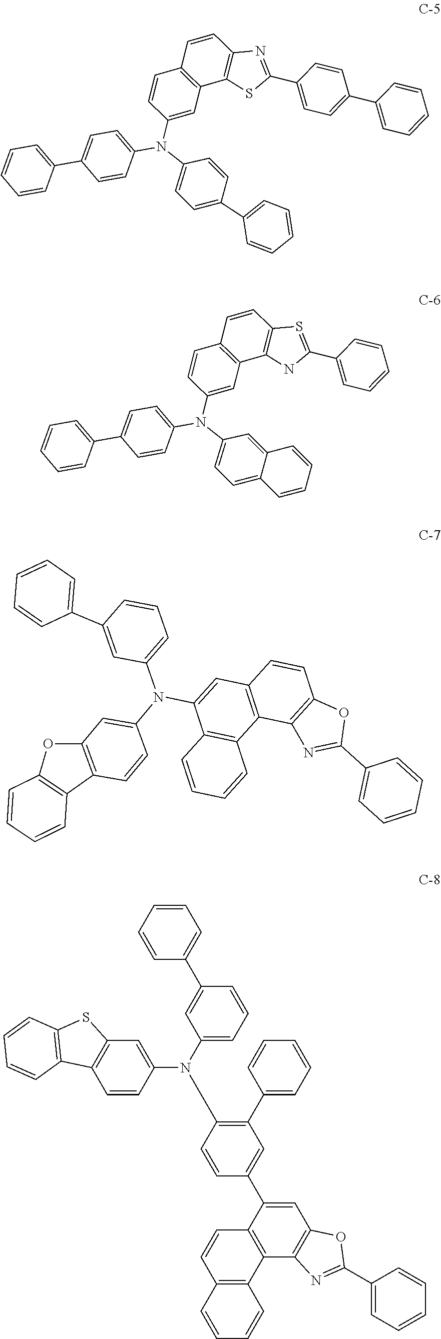

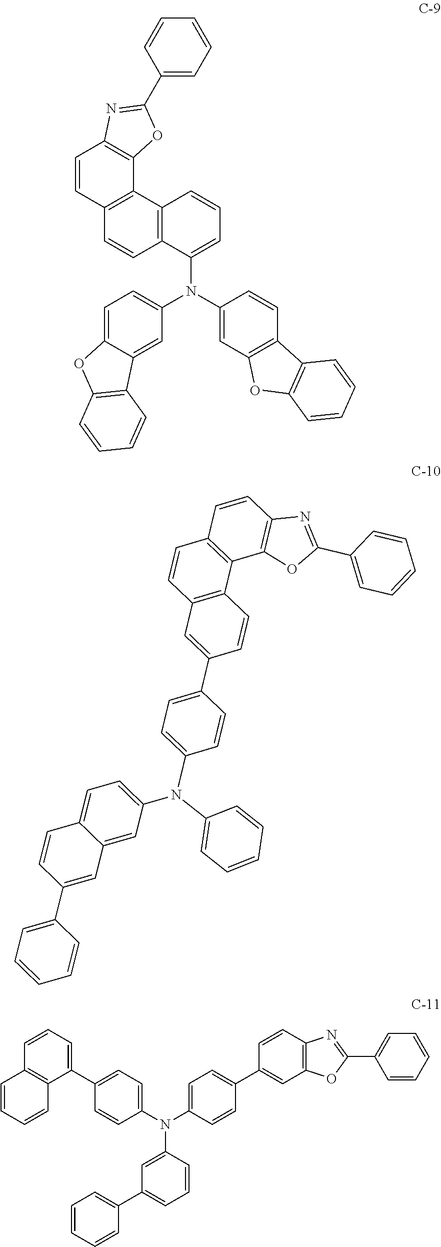

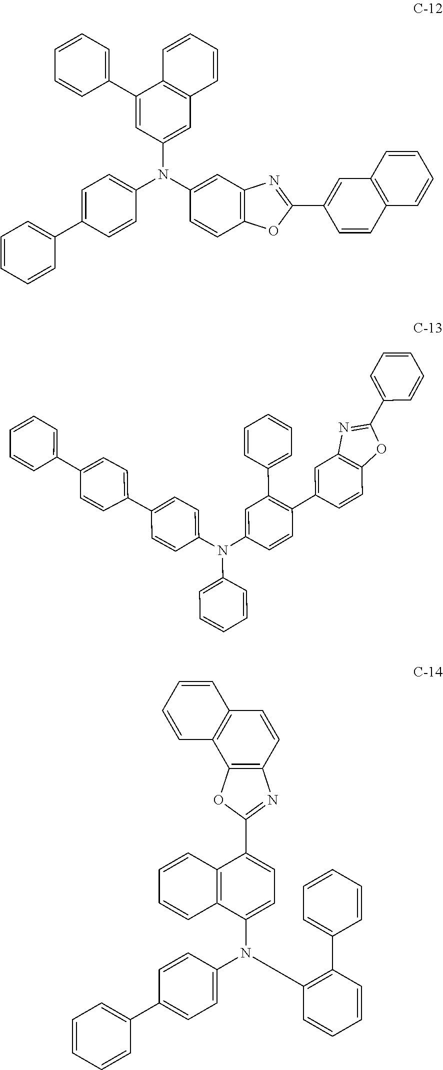 [SMILES: c1ccc(-c2ccc(N(c3cc(-c4ccccc4)c4ccccc4c3)c3ccc4oc(-c5ccc6ccccc6c5)nc4c3)cc2)cc1 ; c1ccc(-c2ccc(-c3ccc(N(c4ccccc4)c4ccc(-c5ccc6oc(-c7ccccc7)nc6c5)c(-c5ccccc5)c4)cc3)cc2)cc1 ; c1ccc(-c2ccc(N(Cc3ccccc3-c3ccccc3)c3ccc(-c4nc5ccc6ccccc6c5o4)c4ccccc34)cc2)cc1]

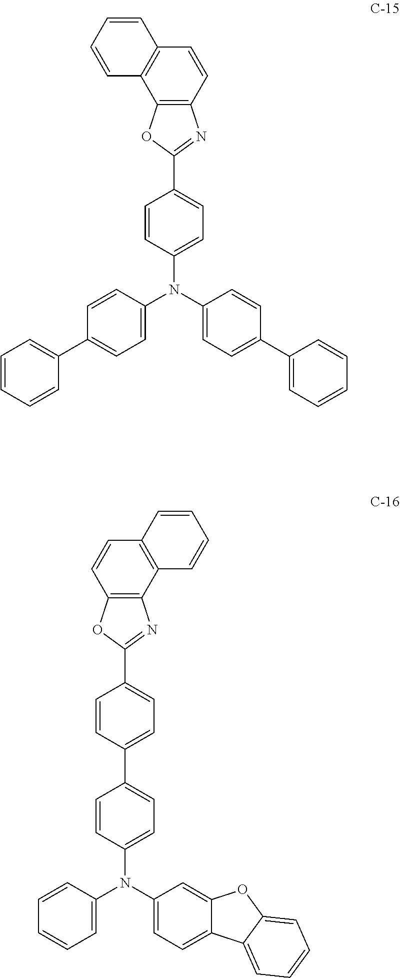 [SMILES: c1ccc(-c2ccc(N(c3ccc(-c4ccccc4)cc3)c3ccc(-c4nc5ccc6ccccc6c5o4)cc3)cc2)cc1 ; c1ccc(N(c2ccc(-c3ccc(-c4nc5c(ccc6ccccc65)o4)cc3)cc2)c2ccc3c(c2)oc2ccccc23)cc1]

For the organic light emitting devices prepared in Experimental Examples and Experimental Comparative Examples, the voltage and efficiency were measured by applying a current (15 mA/cm²), and the results are shown in Tables 1 to 6 below. In addition, lifespan (T95) means the time (hr) taken until the initial luminance (6000 nit) decreases to 95%.

TABLE 1

| | Electron | Driving | Efficiency | Lifespan | Emission |
|---|---|---|---|---|---|
| | blocking layer | voltage (V) | (cd/A) | T95(hr) | color |
| Experimental Ex. 1 | Compound 1 | 3.83 | 19.65 | 192 | Red |
| Experimental Ex. 2 | Compound 2 | 3.78 | 19.30 | 193 | Red |
| Experimental Ex. 3 | Compound 3 | 3.92 | 19.55 | 195 | Red |
| Experimental Ex. 4 | Compound 4 | 3.83 | 18.97 | 221 | Red |
| Experimental Ex. 5 | Compound 5 | 3.78 | 19.20 | 214 | Red |
| Experimental Ex. 6 | Compound 6 | 3.73 | 18.80 | 222 | Red |
| Experimental Ex. 7 | Compound 7 | 3.82 | 19.16 | 193 | Red |
| Experimental Ex. 8 | Compound 8 | 3.80 | 19.37 | 190 | Red |
| Experimental Ex. 9 | Compound 9 | 3.84 | 19.43 | 195 | Red |
| Experimental Ex. 10 | Compound 10 | 3.88 | 19.80 | 180 | Red |
| Experimental Ex. 11 | Compound 11 | 3.97 | 20.27 | 234 | Red |
| Experimental Ex. 12 | Compound 12 | 3.88 | 21.83 | 237 | Red |
| Experimental Ex. 13 | Compound 13 | 3.62 | 20.83 | 239 | Red |
| Experimental Ex. 14 | Compound 14 | 3.69 | 22.16 | 236 | Red |
| Experimental Ex. 15 | Compound 15 | 3.73 | 21.47 | 234 | Red |
| Experimental Ex. 16 | Compound 16 | 3.70 | 19.75 | 230 | Red |
| Experimental Ex. 17 | Compound 17 | 3.92 | 21.33 | 241 | Red |
| Experimental Ex. 18 | Compound 18 | 3.88 | 20.85 | 239 | Red |
| Experimental Ex. 19 | Compound 19 | 3.91 | 22.20 | 240 | Red |
| Experimental Ex. 20 | Compound 20 | 3.98 | 21.88 | 241 | Red |

TABLE 2

| | Electron | Driving | Efficiency | Lifespan | Emission |
|---|---|---|---|---|---|
| | blocking layer | voltage (V) | (cd/A) | T95(hr) | color |
| Experimental Ex. 21 | Compound 21 | 3.76 | 18.77 | 211 | Red |
| Experimental Ex. 22 | Compound 22 | 3.82 | 19.12 | 229 | Red |
| Experimental Ex. 23 | Compound 23 | 3.70 | 19.10 | 234 | Red |
| Experimental Ex. 24 | Compound 24 | 3.75 | 19.28 | 230 | Red |
| Experimental Ex. 25 | Compound 25 | 3.70 | 18.64 | 233 | Red |
| Experimental Ex. 26 | Compound 26 | 3.74 | 18.71 | 227 | Red |
| Experimental Ex. 27 | Compound 27 | 3.82 | 19.04 | 232 | Red |
| Experimental Ex. 28 | Compound 28 | 3.73 | 18.82 | 228 | Red |
| Experimental Ex. 29 | Compound 29 | 3.76 | 19.07 | 217 | Red |
| Experimental Ex. 30 | Compound 30 | 3.76 | 18.95 | 222 | Red |
| Experimental Ex. 31 | Compound 31 | 3.88 | 19.27 | 192 | Red |
| Experimental Ex. 32 | Compound 32 | 3.86 | 19.61 | 190 | Red |
| Experimental Ex. 33 | Compound 33 | 3.82 | 19.29 | 191 | Red |
| Experimental Ex. 34 | Compound 34 | 3.72 | 18.75 | 218 | Red |
| Experimental Ex. 35 | Compound 35 | 3.90 | 19.17 | 193 | Red |
| Experimental Ex. 36 | Compound 36 | 3.80 | 19.14 | 185 | Red |
| Experimental Ex. 37 | Compound 37 | 3.68 | 18.58 | 227 | Red |
| Experimental Ex. 38 | Compound 38 | 3.90 | 19.21 | 194 | Red |
| Experimental Ex. 39 | Compound 39 | 3.77 | 19.25 | 215 | Red |
| Experimental Ex. 40 | Compound 40 | 3.62 | 19.54 | 232 | Red |

TABLE 3

| | Electron | Driving | Efficiency | Lifespan | Emission |
|---|---|---|---|---|---|
| | blocking layer | voltage (V) | (cd/A) | T95(hr) | color |
| Experimental Ex. 41 | Compound 41 | 3.67 | 18.39 | 213 | Red |
| Experimental Ex. 42 | Compound 42 | 3.66 | 18.62 | 208 | Red |
| Experimental Ex. 43 | Compound 43 | 3.69 | 18.46 | 199 | Red |
| Experimental Ex. 44 | Compound 44 | 3.63 | 18.65 | 191 | Red |
| Experimental Ex. 45 | Compound 45 | 3.69 | 18.29 | 204 | Red |
| Experimental Ex. 46 | Compound 46 | 3.71 | 20.97 | 235 | Red |
| Experimental Ex. 47 | Compound 47 | 3.66 | 18.56 | 198 | Red |
| Experimental Ex. 48 | Compound 48 | 3.63 | 18.41 | 195 | Red |
| Experimental Ex. 49 | Compound 49 | 3.71 | 18.64 | 213 | Red |
| Experimental Ex. 50 | Compound 50 | 3.70 | 18.70 | 209 | Red |
| Experimental Ex. 51 | Compound 51 | 3.90 | 19.33 | 183 | Red |
| Experimental Ex. 52 | Compound 52 | 3.87 | 19.27 | 192 | Red |
| Experimental Ex. 53 | Compound 53 | 3.79 | 19.66 | 184 | Red |
| Experimental Ex. 54 | Compound 54 | 3.85 | 19.49 | 182 | Red |
| Experimental Ex. 55 | Compound 55 | 3.76 | 18.79 | 225 | Red |
| Experimental Ex. 56 | Compound 56 | 3.87 | 19.62 | 180 | Red |
| Experimental Ex. 57 | Compound 57 | 3.77 | 19.76 | 191 | Red |
| Experimental Ex. 58 | Compound 58 | 3.77 | 19.76 | 186 | Red |
| Experimental Ex. 59 | Compound 59 | 3.70 | 20.80 | 244 | Red |
| Experimental Ex. 60 | Compound 60 | 3.83 | 19.40 | 195 | Red |

TABLE 4

| | Electron | Driving | Efficiency | Lifespan | Emission |
|---|---|---|---|---|---|
| | blocking layer | voltage (V) | (cd/A) | T95(hr) | color |
| Experimental Ex. 61 | Compound 61 | 3.73 | 19.13 | 229 | Red |
| Experimental Ex. 62 | Compound 62 | 3.71 | 18.93 | 230 | Red |
| Experimental Ex. 63 | Compound 63 | 3.73 | 18.83 | 222 | Red |
| Experimental Ex. 64 | Compound 64 | 3.72 | 20.63 | 242 | Red |
| Experimental Ex. 65 | Compound 65 | 3.74 | 19.13 | 226 | Red |
| Experimental Ex. 66 | Compound 66 | 3.68 | 19.28 | 228 | Red |
| Experimental Ex. 67 | Compound 67 | 3.72 | 19.21 | 225 | Red |
| Experimental Ex. 68 | Compound 68 | 3.73 | 19.20 | 227 | Red |
| Experimental Ex. 69 | Compound 69 | 3.76 | 18.69 | 228 | Red |
| Experimental Ex. 70 | Compound 70 | 3.76 | 18.55 | 218 | Red |
| Experimental Ex. 71 | Compound 71 | 3.98 | 20.33 | 233 | Red |
| Experimental Ex. 72 | Compound 72 | 3.92 | 19.52 | 232 | Red |
| Experimental Ex. 73 | Compound 73 | 3.98 | 19.91 | 229 | Red |
| Experimental Ex. 74 | Compound 74 | 3.97 | 21.15 | 242 | Red |
| Experimental Ex. 75 | Compound 75 | 3.89 | 19.55 | 231 | Red |
| Experimental Ex. 76 | Compound 76 | 3.95 | 20.12 | 246 | Red |
| Experimental Ex. 77 | Compound 77 | 3.89 | 22.10 | 247 | Red |
| Experimental Ex. 78 | Compound 78 | 3.95 | 20.63 | 248 | Red |
| Experimental Ex. 79 | Compound 79 | 3.65 | 22.23 | 234 | Red |
| Experimental Ex. 80 | Compound 80 | 3.88 | 20.57 | 233 | Red |

TABLE 5

| | Electron | Driving | Efficiency | Lifespan | Emission |
|---|---|---|---|---|---|
| | blocking layer | voltage (V) | (cd/A) | T95(hr) | color |
| Experimental Ex. 81 | Compound 81 | 3.64 | 19.69 | 241 | Red |
| Experimental Ex. 82 | Compound 82 | 3.70 | 21.03 | 243 | Red |
| Experimental Ex. 83 | Compound 83 | 3.73 | 19.67 | 228 | Red |
| Experimental Ex. 84 | Compound 84 | 3.63 | 21.06 | 248 | Red |
| Experimental Ex. 85 | Compound 85 | 3.74 | 22.06 | 229 | Red |
| Experimental Ex. 86 | Compound 86 | 3.63 | 19.53 | 241 | Red |
| Experimental Ex. 87 | Compound 87 | 3.65 | 20.37 | 242 | Red |
| Experimental Ex. 88 | Compound 88 | 3.62 | 21.25 | 244 | Red |
| Experimental Ex. 89 | Compound 89 | 3.64 | 22.04 | 236 | Red |
| Experimental Ex. 90 | Compound 90 | 3.71 | 22.11 | 230 | Red |
| Experimental Ex. 91 | Compound 91 | 3.63 | 18.49 | 195 | Red |
| Experimental Ex. 92 | Compound 92 | 3.63 | 18.36 | 209 | Red |
| Experimental Ex. 93 | Compound 93 | 3.72 | 19.72 | 245 | Red |
| Experimental Ex. 94 | Compound 94 | 3.69 | 18.68 | 196 | Red |
| Experimental Ex. 95 | Compound 95 | 3.70 | 18.30 | 201 | Red |
| Experimental Ex. 96 | Compound 96 | 3.70 | 18.04 | 212 | Red |
| Experimental Ex. 97 | Compound 97 | 3.65 | 18.36 | 209 | Red |
| Experimental Ex. 98 | Compound 98 | 3.74 | 18.29 | 198 | Red |
| Experimental Ex. 99 | Compound 99 | 3.69 | 18.28 | 196 | Red |
| Experimental Ex. 100 | Compound 100 | 3.64 | 18.63 | 208 | Red |
| Experimental Ex. 101 | Compound 101 | 3.90 | 19.46 | 186 | Red |
| Experimental Ex. 102 | Compound 102 | 3.90 | 19.37 | 183 | Red |
| Experimental Ex. 103 | Compound 103 | 3.91 | 19.65 | 192 | Red |
| Experimental Ex. 104 | Compound 104 | 3.88 | 19.62 | 192 | Red |
| Experimental Ex. 105 | Compound 105 | 3.93 | 19.29 | 188 | Red |

TABLE 6

| | Electron | Driving | Efficiency | Lifespan | Emission |
|---|---|---|---|---|---|
| | blocking layer | voltage (V) | (cd/A) | T95(hr) | color |
| Comparative | Compound C-1 | 4.11 | 17.13 | 165 | Red |
| Experimental Ex. 1 | | | | | |
| Comparative | Compound C-2 | 4.07 | 17.17 | 168 | Red |
| Experimental Ex. 2 | | | | | |
| Comparative | Compound C-3 | 4.06 | 17.29 | 150 | Red |
| Experimental Ex. 3 | | | | | |
| Comparative | Compound C-4 | 4.26 | 16.17 | 93 | Red |
| Experimental Ex. 4 | | | | | |
| Comparative | Compound C-5 | 4.22 | 15.29 | 99 | Red |
| Experimental Ex. 5 | | | | | |
| Comparative | Compound C-6 | 4.29 | 15.31 | 109 | Red |
| Experimental Ex. 6 | | | | | |
| Comparative | Compound C-7 | 4.11 | 17.16 | 158 | Red |
| Experimental Ex. 7 | | | | | |
| Comparative | Compound C-8 | 4.07 | 17.15 | 161 | Red |
| Experimental Ex. 8 | | | | | |
| Comparative | Compound C-9 | 4.11 | 16.95 | 167 | Red |
| Experimental Ex. 9 | | | | | |
| Comparative | Compound C-10 | 4.12 | 17.09 | 168 | Red |
| Experimental Ex. 10 | | | | | |
| Comparative | Compound C-11 | 4.28 | 15.22 | 103 | Red |
| Experimental Ex. 11 | | | | | |
| Comparative | Compound C-12 | 4.27 | 16.00 | 96 | Red |
| Experimental Ex. 12 | | | | | |
| Comparative | Compound C-13 | 4.31 | 15.45 | 101 | Red |
| Experimental Ex. 13 | | | | | |
| Comparative | Compound C-14 | 4.13 | 16.74 | 107 | Red |
| Experimental Ex. 14 | | | | | |
| Comparative | Compound C-15 | 4.07 | 16.96 | 138 | Red |
| Experimental Ex. 15 | | | | | |
| Comparative | Compound C-16 | 4.06 | 16.44 | 123 | Red |
| Experimental Ex. 16 | | | | | |

When a current was applied to the organic light emitting devices manufactured according to Experimental Examples 1 to 105 and Comparative Experimental Examples 1 to 16, the results shown in Tables 1 to 6 were obtained. The red organic light emitting device of Experimental Example 1 used a material widely used in the prior art, and has a structure using Dp-7 as a dopant for the red light-emitting layer. In Comparative Experimental Examples 1 to 16, organic light emitting devices were prepared by using Compounds C-1 to C-16 instead of Compound 1. Referring to the results of Table 1, when the compound of the present disclosure was used as the electron blocking layer, the driving voltage was significantly lowered and the efficiency was increased compared to the case of using the material of Comparative Examples, so it was found that the energy transfer from the host to the red dopant was excellent. In addition, it was found that the lifespan could be greatly improved while maintaining high efficiency. This may be because the compound of the present disclosure has higher stability to electrons and holes than the compound of Comparative Examples. In conclusion, it can be confirmed that when the compound of the present disclosure is used as the electron blocking layer of the red light emitting layer, the driving voltage, luminous efficiency, and lifespan of the organic light emitting device can be improved.

### DESCRIPTION OF REFERENCE NUMERALS

1: Substrate
2: Anode
3: Light emitting layer
4: Cathode
5: Hole injection layer
6: Hole transport layer
7: Electron Blocking Layer
8: Electron transport layer

1. A compound represented by the following Chemical Formula 1 or 2: 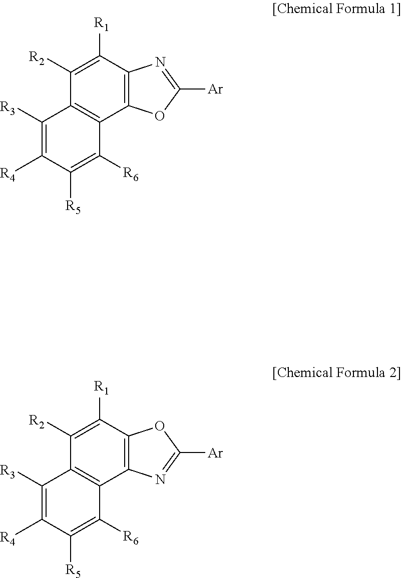
in Chemical Formulae 1 and 2,
Ar is a substituted or unsubstituted C₆₋₆₀ aryl, and
one of R₁ to R₆ is the following Chemical Formula 3, and the rest are each independently hydrogen or deuterium, 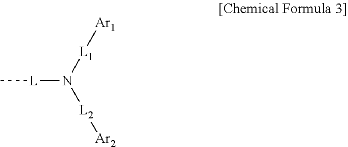
in Chemical Formula 3,
L is a single bond, a substituted or unsubstituted C₆₋₆₀ arylene, or a substituted or unsubstituted C₂₋₆₀ heteroarylene containing at least one selected from the group consisting of N, O and S,
L₁ and L₂ are each independently a single bond, a substituted or unsubstituted C₆₋₆₀ arylene, or a substituted or unsubstituted C₂₋₆₀ heteroarylene containing at least one selected from the group consisting of N, O and S, and
Ar₁ and Ar₂ are each independently a substituted or unsubstituted C₆₋₆₀ aryl, or a substituted or unsubstituted C₂₋₆₀ heteroaryl containing at least one selected from the group consisting of N, O and S,
provided that when R₅ or R₆ is the substituent represented by the Chemical Formula 3,
L₁ is a substituted or unsubstituted C₆₋₆₀ arylene, and Ar₁ is a substituted or unsubstituted C₈₋₆₀ aryl; or
L₁ is a single bond, or a substituted or unsubstituted C₆₋₆₀ arylene, and Ar₁ is a substituted or unsubstituted C₂₋₆₀ heteroaryl containing at least one selected from the group consisting of N, O and S.

2. The compound of claim 1,
wherein Ar is phenyl, biphenyl, or naphthyl.

3. The compound of claim 1,
wherein L is a single bond, phenylene, biphenyldiyl, terphenyldiyl, naphthylene, or -(phenylene)-(naphthylene)-.

4. The compound of claim 1,
wherein L₁ and L₂ are each independently a single bond, phenylene, or biphenyldiyl.

5. The compound of claim 1,
wherein Ar₁ and Ar₂ are each independently phenyl, biphenylyl, terphenylyl, naphthyl, naphthylphenyl, phenylnaphthyl, phenanthrenyl, dimethylfluorenyl, diphenylfluorenyl, dibenzofuranyl, dibenzothiophenyl, 9H-carbazol-9-yl, or 9-phenyl-9H-carbazolyl.

6. The compound of claim 1,
wherein one of R₁ to R₄ is the Chemical Formula 3, and the rest are each independently hydrogen; or deuterium; and
R₅ and R₆ are each independently hydrogen; or deuterium.

7. The compound of claim 1,
wherein R₁ to R₄ are each independently hydrogen or deuterium, and
one of R₅ and R₆ is the Chemical Formula 3, and the rest is hydrogen or deuterium.

8. The compound of claim 7,
wherein L₁ is phenylene or biphenyldiyl, and
Ar₁ is biphenylyl, terphenylyl, naphthyl, phenanthrenyl, dimethylfluorenyl, or diphenylfluorenyl.

9. The compound of claim 7,
wherein L₁ is a single bond, phenylene, or biphenyldiyl, and
Ar₁ is dibenzofuranyl, dibenzothiophenyl, 9H-carbazol-9-yl, or 9-phenyl-9H-carbazolyl.

10. The compound of claim 1,
wherein the compound represented by the Chemical Formula 1 or 2 is one selected from the following:

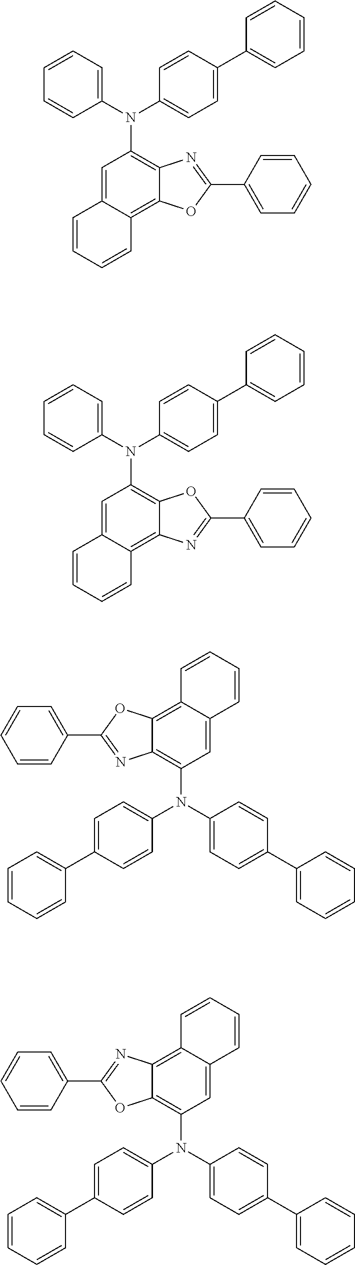 [SMILES: c1ccc(-c2ccc(N(c3ccccc3)c3cc4ccccc4c4oc(-c5ccccc5)nc34)cc2)cc1 ; c1ccc(-c2ccc(N(c3ccccc3)c3cc4ccccc4c4nc(-c5ccccc5)oc34)cc2)cc1 ; c1ccc(-c2ccc(N(c3ccc(-c4ccccc4)cc3)c3cc4ccccc4c4oc(-c5ccccc5)nc34)cc2)cc1 ; c1ccc(-c2ccc(N(c3ccc(-c4ccccc4)cc3)c3cc4ccccc4c4nc(-c5ccccc5)oc34)cc2)cc1]

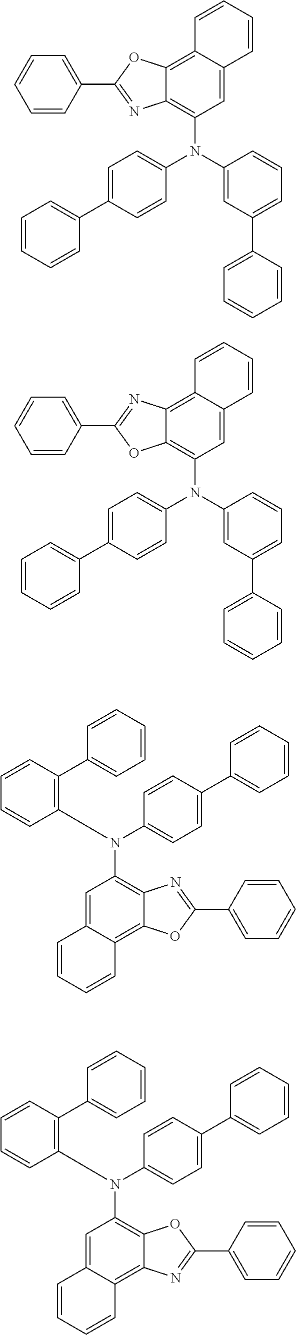

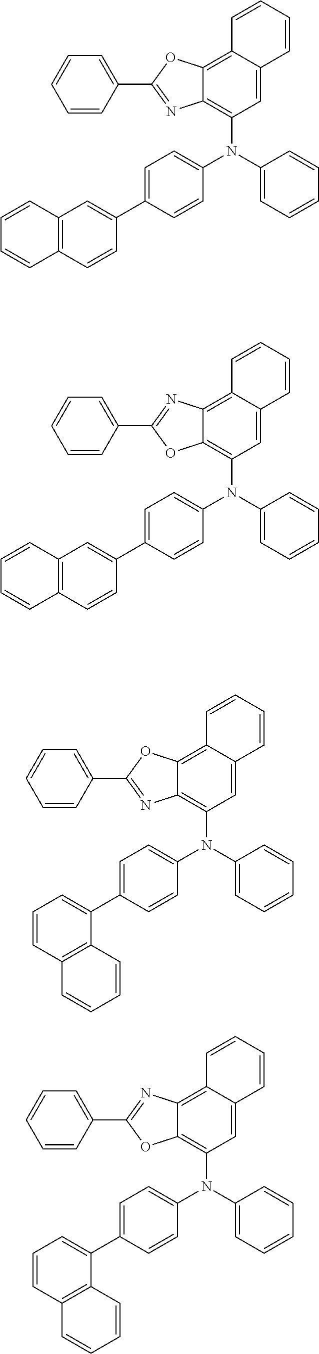 [SMILES: c1ccc(-c2nc3c(N(c4ccccc4)c4ccc(-c5ccc6ccccc6c5)cc4)cc4ccccc4c3o2)cc1 ; c1ccc(-c2nc3c(o2)c(N(c2ccccc2)c2ccc(-c4ccc5ccccc5c4)cc2)cc2ccccc23)cc1 ; c1ccc(-c2nc3c(N(c4ccccc4)c4ccc(-c5cccc6ccccc56)cc4)cc4ccccc4c3o2)cc1 ; c1ccc(-c2nc3c(o2)c(N(c2ccccc2)c2ccc(-c4cccc5ccccc45)cc2)cc2ccccc23)cc1]

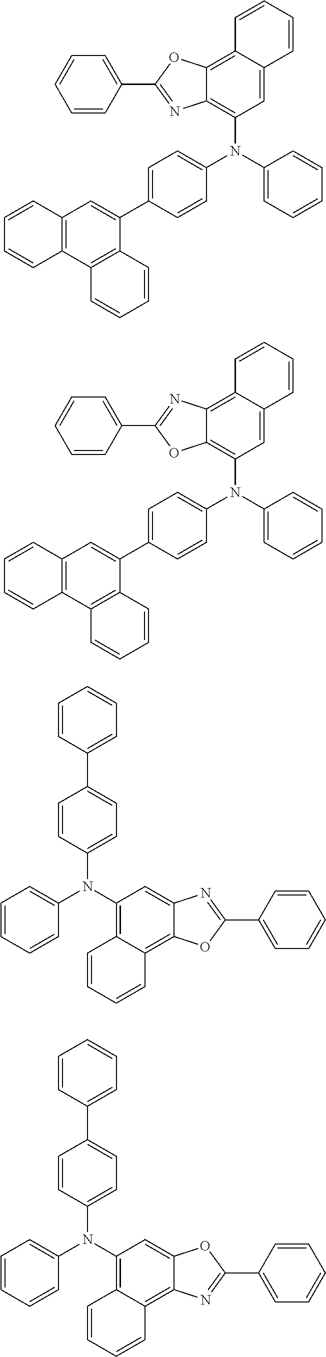

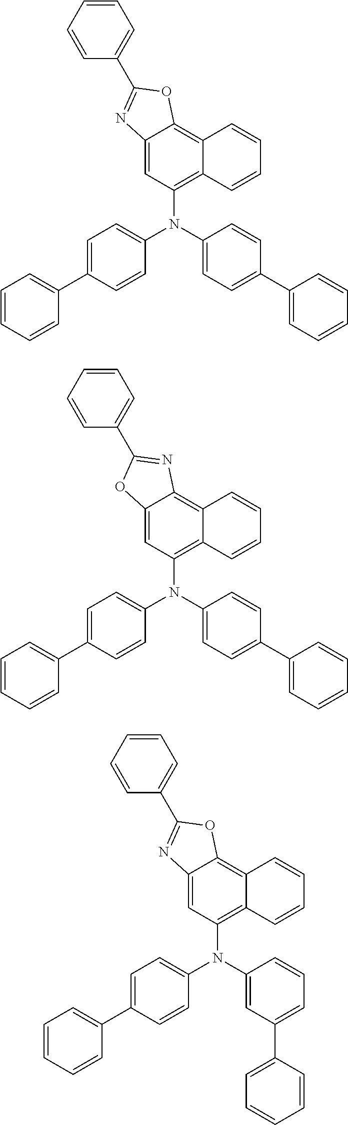 [SMILES: c1ccc(-c2ccc(N(c3ccc(-c4ccccc4)cc3)c3cc4nc(-c5ccccc5)oc4c4ccccc34)cc2)cc1 ; c1ccc(-c2ccc(N(c3ccc(-c4ccccc4)cc3)c3cc4oc(-c5ccccc5)nc4c4ccccc34)cc2)cc1 ; c1ccc(-c2ccc(N(c3cccc(-c4ccccc4)c3)c3cc4nc(-c5ccccc5)oc4c4ccccc34)cc2)cc1]

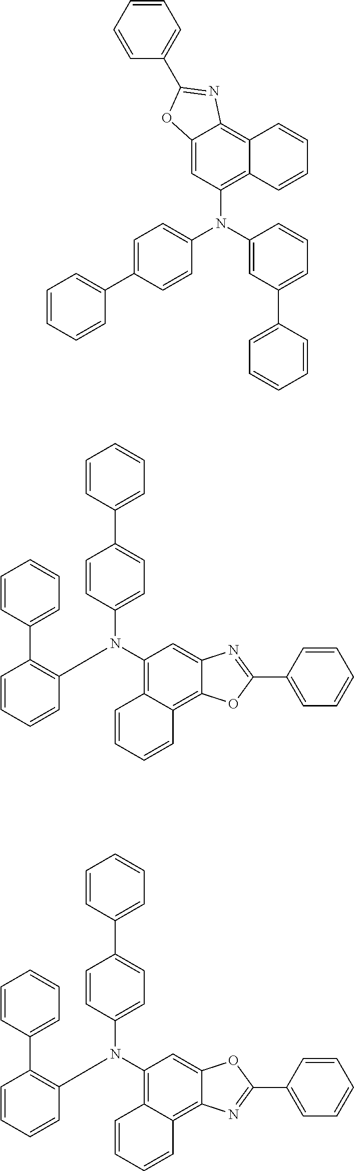 [SMILES: c1ccc(-c2ccc(N(c3cccc(-c4ccccc4)c3)c3cc4oc(-c5ccccc5)nc4c4ccccc34)cc2)cc1 ; c1ccc(-c2ccc(N(c3ccccc3-c3ccccc3)c3cc4nc(-c5ccccc5)oc4c4ccccc34)cc2)cc1 ; c1ccc(-c2ccc(N(c3ccccc3-c3ccccc3)c3cc4oc(-c5ccccc5)nc4c4ccccc34)cc2)cc1]

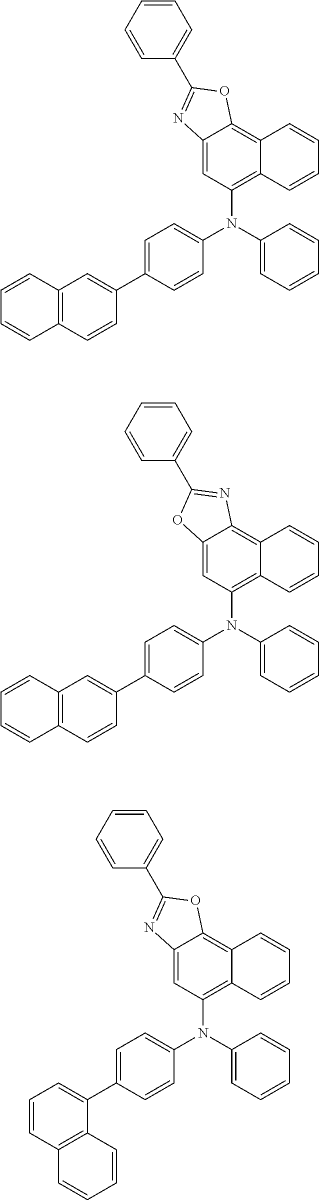 [SMILES: c1ccc(-c2nc3cc(N(c4ccccc4)c4ccc(-c5ccc6ccccc6c5)cc4)c4ccccc4c3o2)cc1 ; c1ccc(-c2nc3c(cc(N(c4ccccc4)c4ccc(-c5ccc6ccccc6c5)cc4)c4ccccc43)o2)cc1 ; c1ccc(-c2nc3cc(N(c4ccccc4)c4ccc(-c5cccc6ccccc56)cc4)c4ccccc4c3o2)cc1]

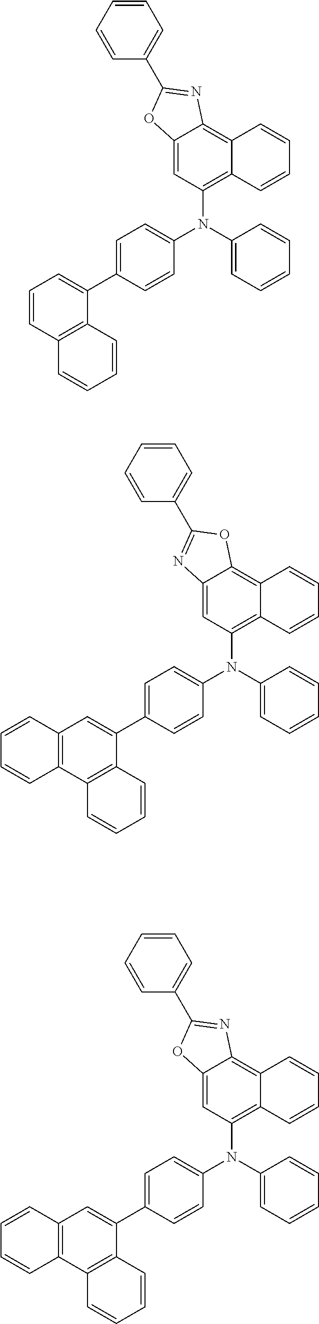 [SMILES: c1ccc(-c2nc3c(cc(N(c4ccccc4)c4ccc(-c5cccc6ccccc56)cc4)c4ccccc43)o2)cc1 ; c1ccc(-c2nc3cc(N(c4ccccc4)c4ccc(-c5cc6ccccc6c6ccccc56)cc4)c4ccccc4c3o2)cc1 ; c1ccc(-c2nc3c(cc(N(c4ccccc4)c4ccc(-c5cc6ccccc6c6ccccc56)cc4)c4ccccc43)o2)cc1]

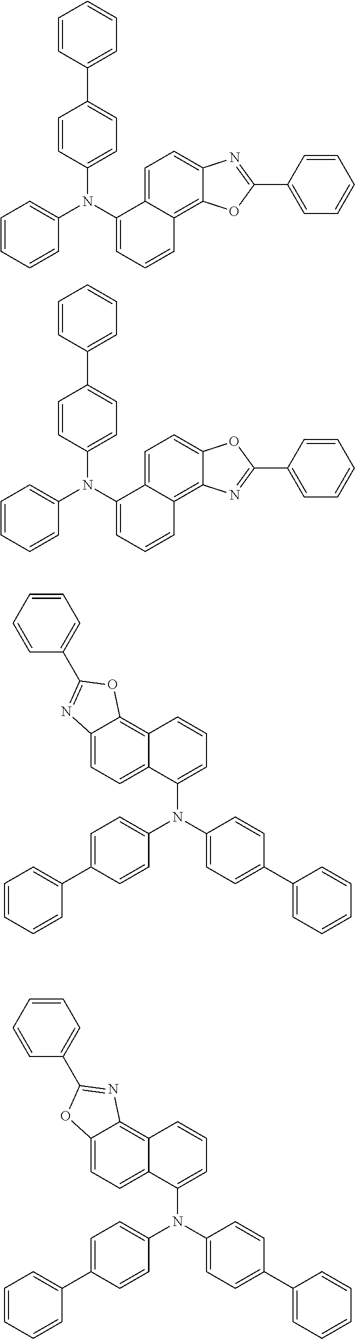

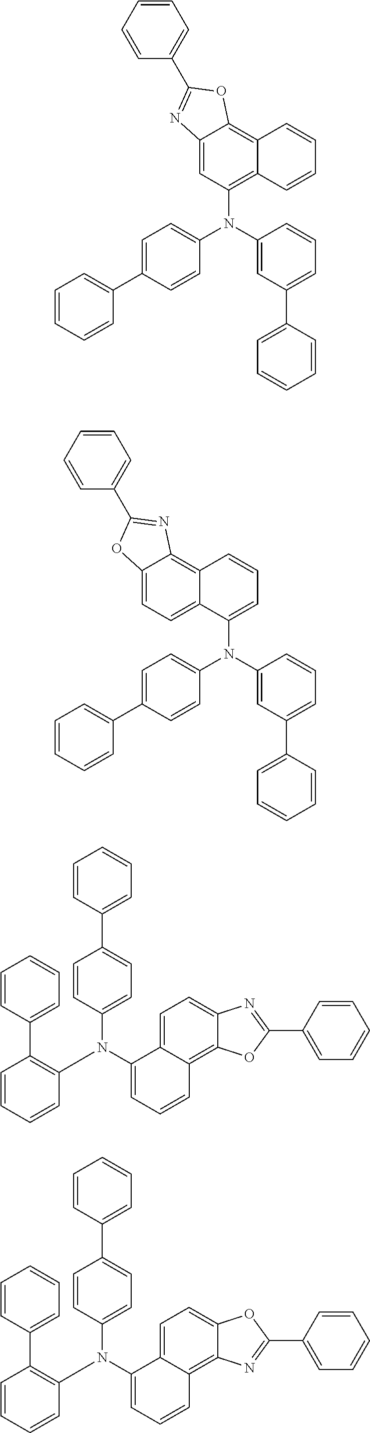 [SMILES: c1ccc(-c2ccc(N(c3cccc(-c4ccccc4)c3)c3cc4nc(-c5ccccc5)oc4c4ccccc34)cc2)cc1 ; c1ccc(-c2ccc(N(c3cccc(-c4ccccc4)c3)c3cccc4c3ccc3oc(-c5ccccc5)nc34)cc2)cc1 ; c1ccc(-c2ccc(N(c3ccccc3-c3ccccc3)c3cccc4c3ccc3nc(-c5ccccc5)oc34)cc2)cc1 ; c1ccc(-c2ccc(N(c3ccccc3-c3ccccc3)c3cccc4c3ccc3oc(-c5ccccc5)nc34)cc2)cc1]

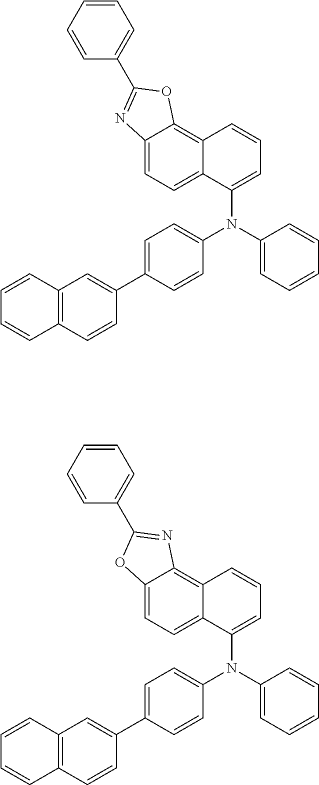 [SMILES: c1ccc(-c2nc3ccc4c(N(c5ccccc5)c5ccc(-c6ccc7ccccc7c6)cc5)cccc4c3o2)cc1 ; c1ccc(-c2nc3c(ccc4c(N(c5ccccc5)c5ccc(-c6ccc7ccccc7c6)cc5)cccc43)o2)cc1]

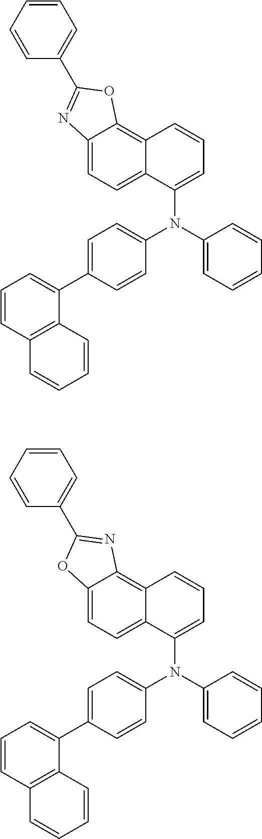 [SMILES: c1ccc(-c2nc3ccc4c(N(c5ccccc5)c5ccc(-c6cccc7ccccc67)cc5)cccc4c3o2)cc1 ; c1ccc(-c2nc3c(ccc4c(N(c5ccccc5)c5ccc(-c6cccc7ccccc67)cc5)cccc43)o2)cc1]

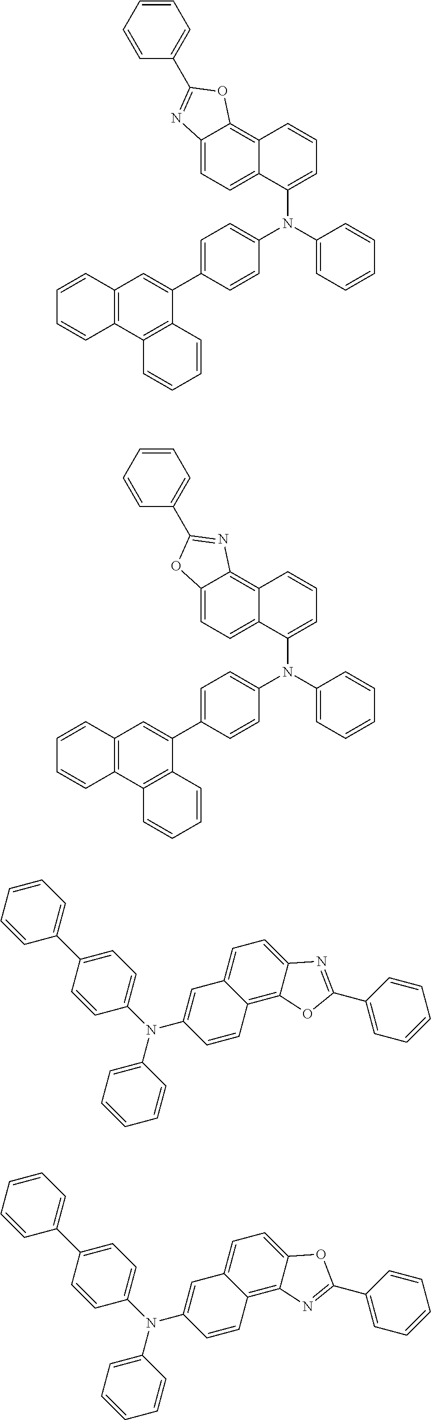 [SMILES: c1ccc(-c2nc3ccc4c(N(c5ccccc5)c5ccc(-c6cc7ccccc7c7ccccc67)cc5)cccc4c3o2)cc1 ; c1ccc(-c2nc3c(ccc4c(N(c5ccccc5)c5ccc(-c6cc7ccccc7c7ccccc67)cc5)cccc43)o2)cc1 ; c1ccc(-c2ccc(N(c3ccccc3)c3ccc4c(ccc5nc(-c6ccccc6)oc54)c3)cc2)cc1 ; c1ccc(-c2ccc(N(c3ccccc3)c3ccc4c(ccc5oc(-c6ccccc6)nc54)c3)cc2)cc1]

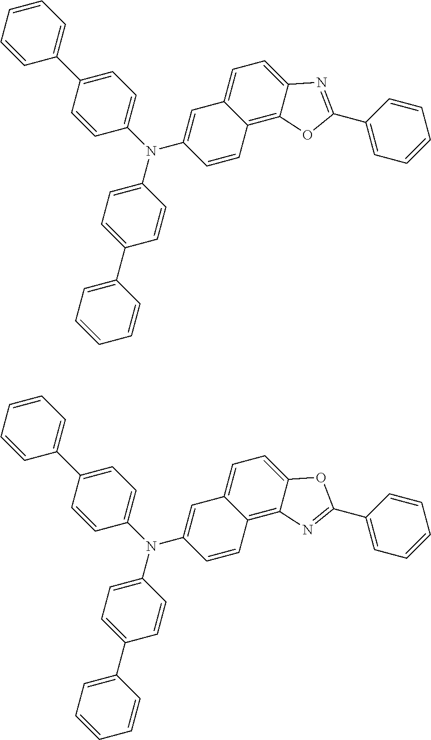 [SMILES: c1ccc(-c2ccc(N(c3ccc(-c4ccccc4)cc3)c3ccc4c(ccc5nc(-c6ccccc6)oc54)c3)cc2)cc1 ; c1ccc(-c2ccc(N(c3ccc(-c4ccccc4)cc3)c3ccc4c(ccc5oc(-c6ccccc6)nc54)c3)cc2)cc1]

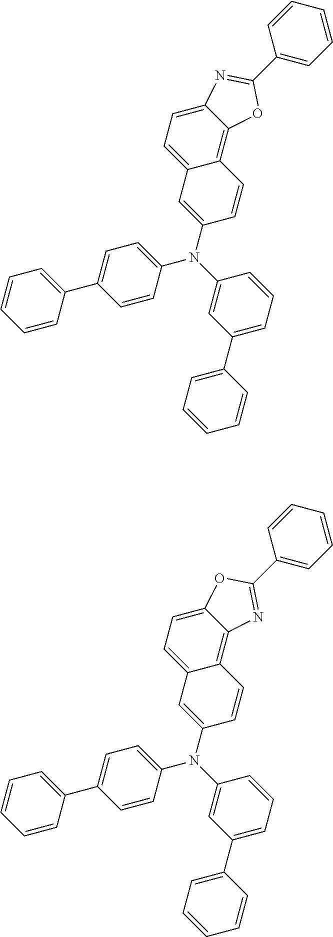 [SMILES: c1ccc(-c2ccc(N(c3cccc(-c4ccccc4)c3)c3ccc4c(ccc5nc(-c6ccccc6)oc54)c3)cc2)cc1 ; c1ccc(-c2ccc(N(c3cccc(-c4ccccc4)c3)c3ccc4c(ccc5oc(-c6ccccc6)nc54)c3)cc2)cc1]

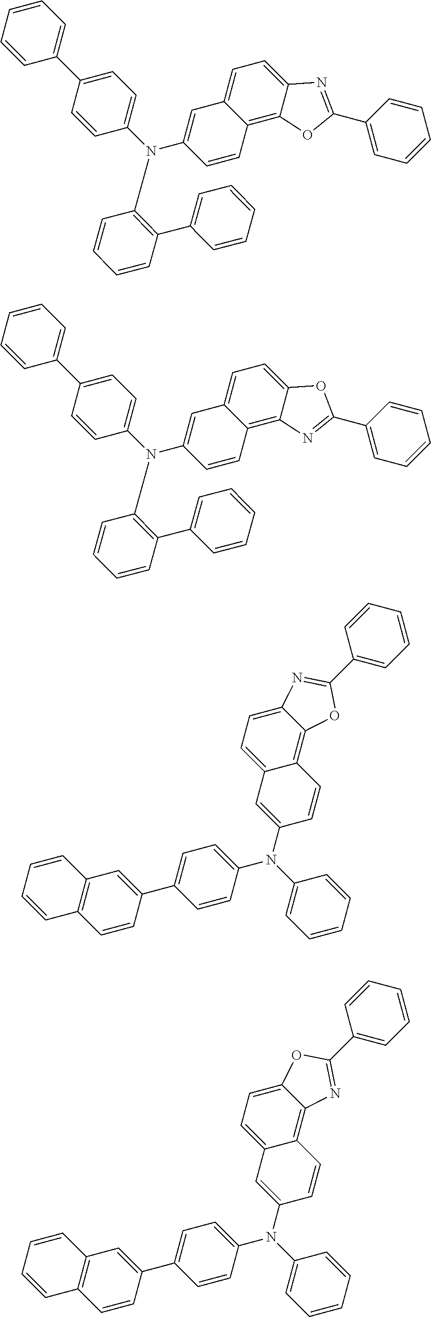 [SMILES: c1ccc(-c2ccc(N(c3ccc4c(ccc5nc(-c6ccccc6)oc54)c3)c3ccccc3-c3ccccc3)cc2)cc1 ; c1ccc(-c2ccc(N(c3ccc4c(ccc5oc(-c6ccccc6)nc54)c3)c3ccccc3-c3ccccc3)cc2)cc1 ; c1ccc(-c2nc3ccc4cc(N(c5ccccc5)c5ccc(-c6ccc7ccccc7c6)cc5)ccc4c3o2)cc1 ; c1ccc(-c2nc3c(ccc4cc(N(c5ccccc5)c5ccc(-c6ccc7ccccc7c6)cc5)ccc43)o2)cc1]

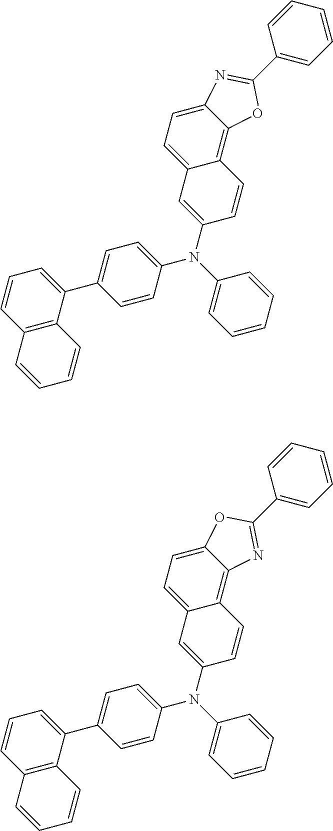 [SMILES: c1ccc(-c2nc3ccc4cc(N(c5ccccc5)c5ccc(-c6cccc7ccccc67)cc5)ccc4c3o2)cc1 ; c1ccc(-c2nc3c(ccc4cc(N(c5ccccc5)c5ccc(-c6cccc7ccccc67)cc5)ccc43)o2)cc1]

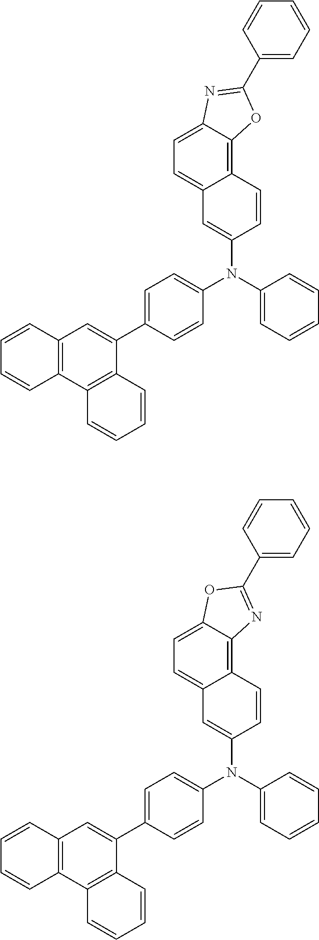 [SMILES: c1ccc(-c2nc3ccc4cc(N(c5ccccc5)c5ccc(-c6cc7ccccc7c7ccccc67)cc5)ccc4c3o2)cc1 ; c1ccc(-c2nc3c(ccc4cc(N(c5ccccc5)c5ccc(-c6cc7ccccc7c7ccccc67)cc5)ccc43)o2)cc1]

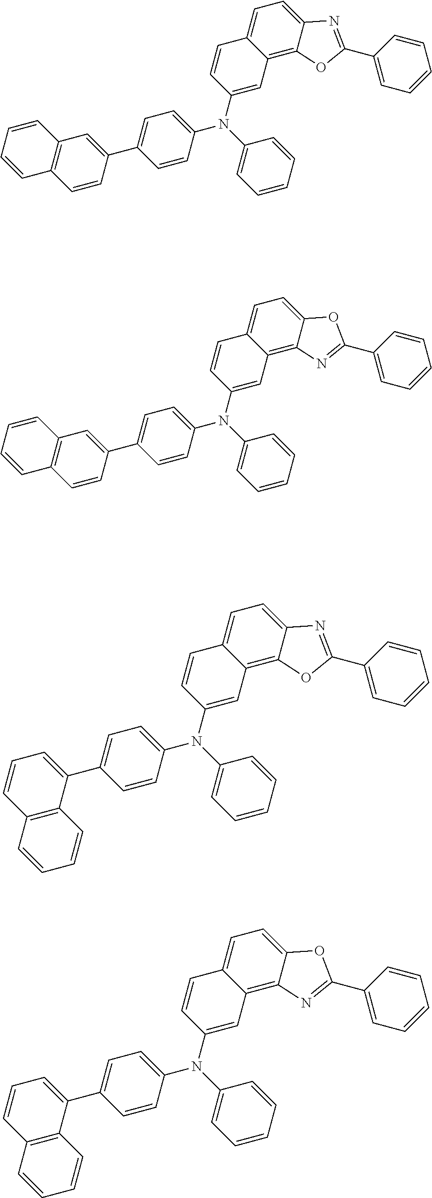 [SMILES: c1ccc(-c2nc3ccc4ccc(N(c5ccccc5)c5ccc(-c6ccc7ccccc7c6)cc5)cc4c3o2)cc1 ; c1ccc(-c2nc3c(ccc4ccc(N(c5ccccc5)c5ccc(-c6ccc7ccccc7c6)cc5)cc43)o2)cc1 ; c1ccc(-c2nc3ccc4ccc(N(c5ccccc5)c5ccc(-c6cccc7ccccc67)cc5)cc4c3o2)cc1 ; c1ccc(-c2nc3c(ccc4ccc(N(c5ccccc5)c5ccc(-c6cccc7ccccc67)cc5)cc43)o2)cc1]

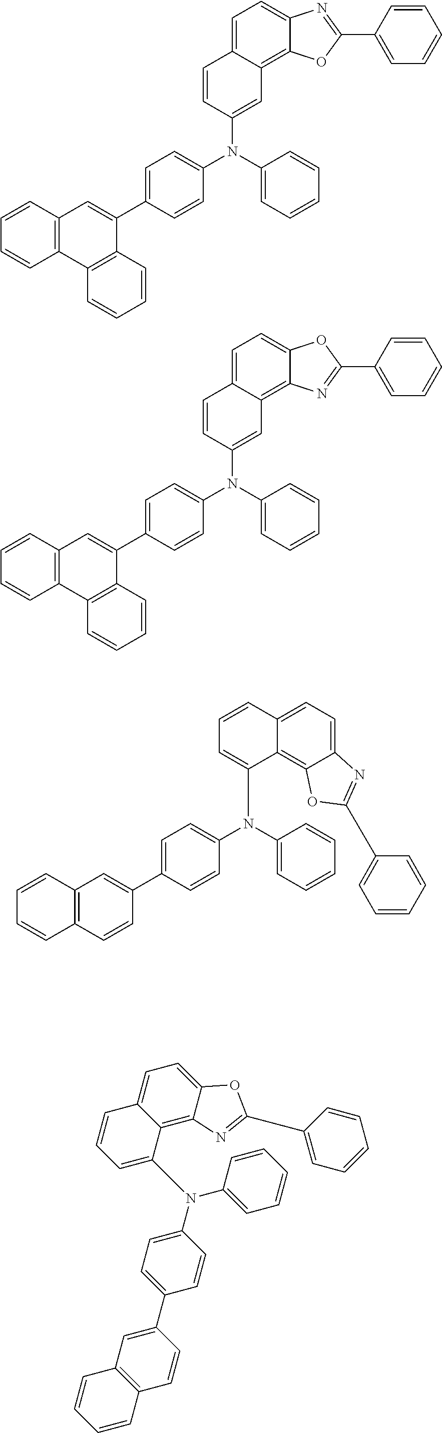 [SMILES: c1ccc(-c2nc3ccc4ccc(N(c5ccccc5)c5ccc(-c6cc7ccccc7c7ccccc67)cc5)cc4c3o2)cc1 ; c1ccc(-c2nc3c(ccc4ccc(N(c5ccccc5)c5ccc(-c6cc7ccccc7c7ccccc67)cc5)cc43)o2)cc1 ; c1ccc(-c2nc3ccc4cccc(N(c5ccccc5)c5ccc(-c6ccc7ccccc7c6)cc5)c4c3o2)cc1 ; c1ccc(-c2nc3c(ccc4cccc(N(c5ccccc5)c5ccc(-c6ccc7ccccc7c6)cc5)c43)o2)cc1]

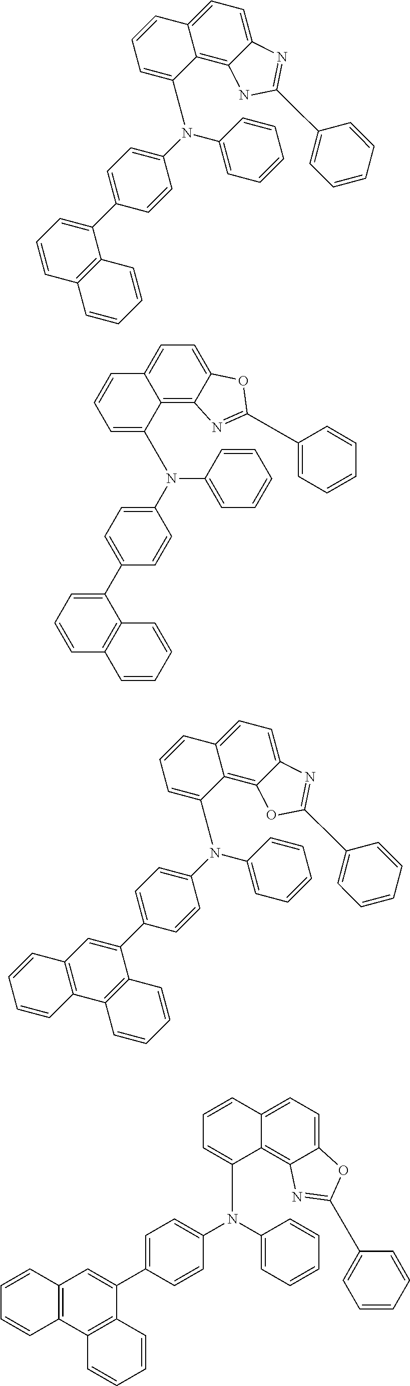 [SMILES: c1ccc(-c2nc3ccc4cccc(N(c5ccccc5)c5ccc(-c6cccc7ccccc67)cc5)c4c3[nH]2)cc1 ; c1ccc(-c2nc3c(ccc4cccc(N(c5ccccc5)c5ccc(-c6cccc7ccccc67)cc5)c43)o2)cc1 ; c1ccc(-c2nc3ccc4cccc(N(c5ccccc5)c5ccc(-c6cc7ccccc7c7ccccc67)cc5)c4c3o2)cc1 ; c1ccc(-c2nc3c(ccc4cccc(N(c5ccccc5)c5ccc(-c6cc7ccccc7c7ccccc67)cc5)c43)o2)cc1]

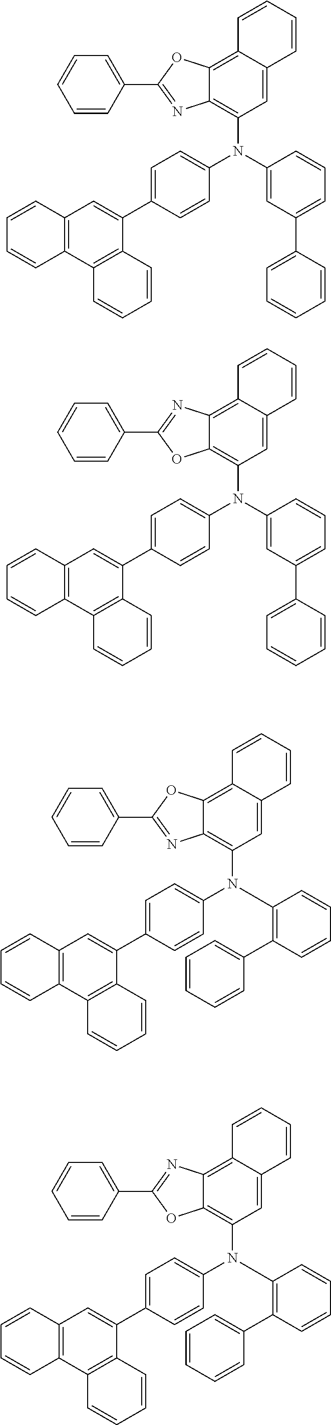

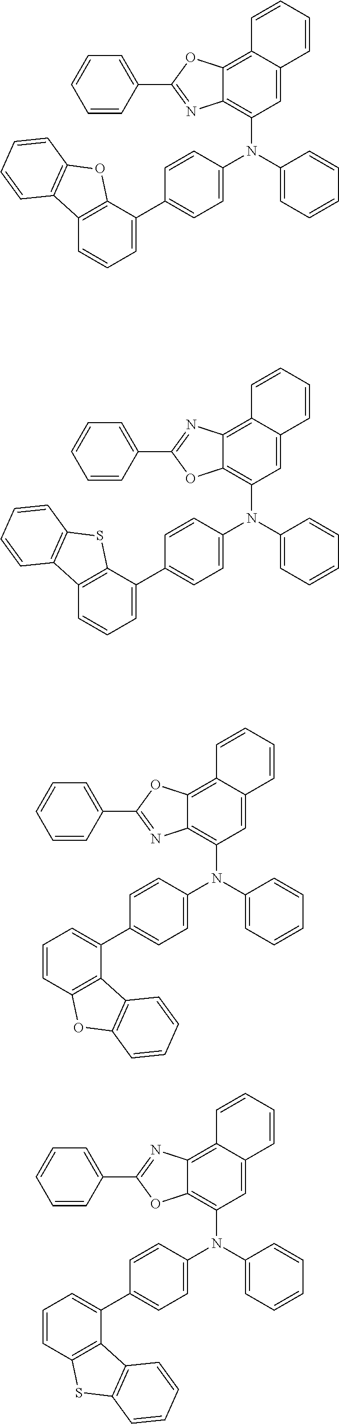 [SMILES: c1ccc(-c2nc3c(N(c4ccccc4)c4ccc(-c5cccc6c5oc5ccccc56)cc4)cc4ccccc4c3o2)cc1 ; c1ccc(-c2nc3c(o2)c(N(c2ccccc2)c2ccc(-c4cccc5c4sc4ccccc45)cc2)cc2ccccc23)cc1 ; c1ccc(-c2nc3c(N(c4ccccc4)c4ccc(-c5cccc6oc7ccccc7c56)cc4)cc4ccccc4c3o2)cc1 ; c1ccc(-c2nc3c(o2)c(N(c2ccccc2)c2ccc(-c4cccc5sc6ccccc6c45)cc2)cc2ccccc23)cc1]

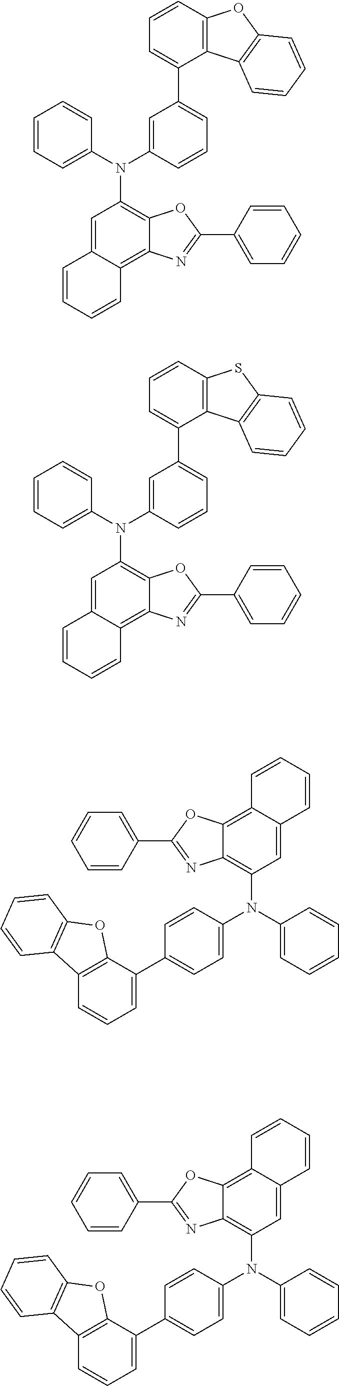 [SMILES: c1ccc(-c2nc3c(o2)c(N(c2ccccc2)c2cccc(-c4cccc5oc6ccccc6c45)c2)cc2ccccc23)cc1 ; c1ccc(-c2nc3c(o2)c(N(c2ccccc2)c2cccc(-c4cccc5sc6ccccc6c45)c2)cc2ccccc23)cc1 ; c1ccc(-c2nc3c(N(c4ccccc4)c4ccc(-c5cccc6c5oc5ccccc56)cc4)cc4ccccc4c3o2)cc1 ; c1ccc(-c2nc3c(N(c4ccccc4)c4ccc(-c5cccc6c5oc5ccccc56)cc4)cc4ccccc4c3o2)cc1]

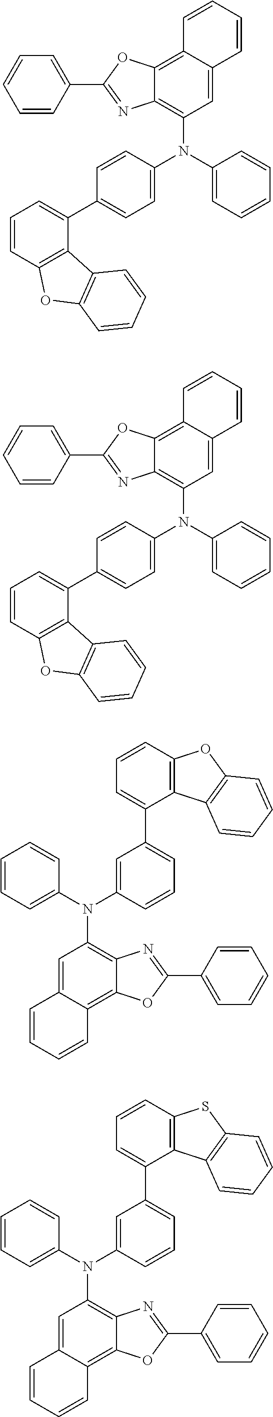

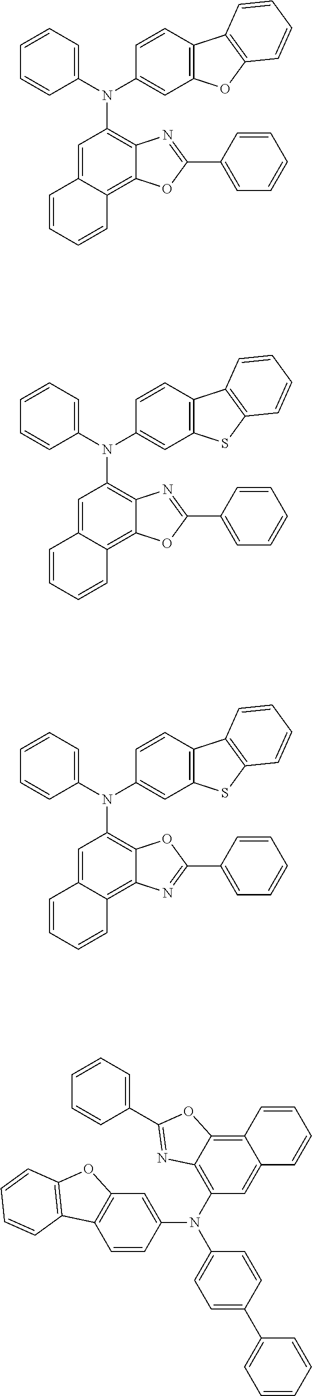 [SMILES: c1ccc(-c2nc3c(N(c4ccccc4)c4ccc5c(c4)oc4ccccc45)cc4ccccc4c3o2)cc1 ; c1ccc(-c2nc3c(N(c4ccccc4)c4ccc5c(c4)sc4ccccc45)cc4ccccc4c3o2)cc1 ; c1ccc(-c2nc3c(o2)c(N(c2ccccc2)c2ccc4c(c2)sc2ccccc24)cc2ccccc23)cc1 ; c1ccc(-c2ccc(N(c3ccc4c(c3)oc3ccccc34)c3cc4ccccc4c4oc(-c5ccccc5)nc34)cc2)cc1]

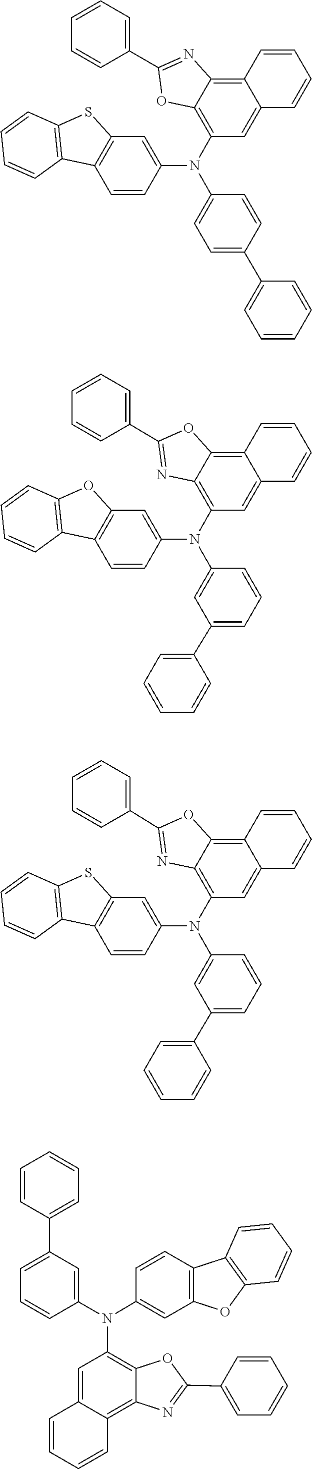 [SMILES: c1ccc(-c2ccc(N(c3ccc4c(c3)sc3ccccc34)c3cc4ccccc4c4nc(-c5ccccc5)oc34)cc2)cc1 ; c1ccc(-c2cccc(N(c3ccc4c(c3)oc3ccccc34)c3cc4ccccc4c4oc(-c5ccccc5)nc34)c2)cc1 ; c1ccc(-c2cccc(N(c3ccc4c(c3)sc3ccccc34)c3cc4ccccc4c4oc(-c5ccccc5)nc34)c2)cc1 ; c1ccc(-c2cccc(N(c3ccc4c(c3)oc3ccccc34)c3cc4ccccc4c4nc(-c5ccccc5)oc34)c2)cc1]

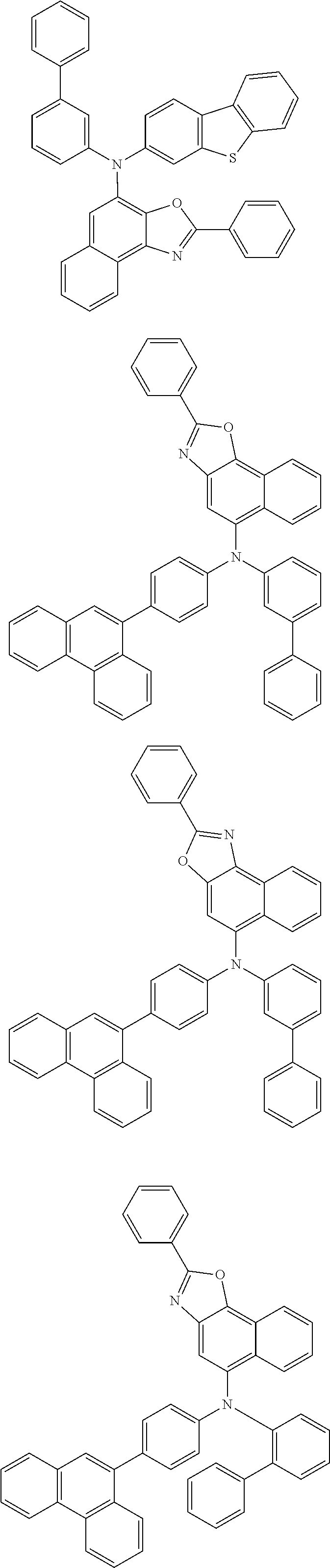

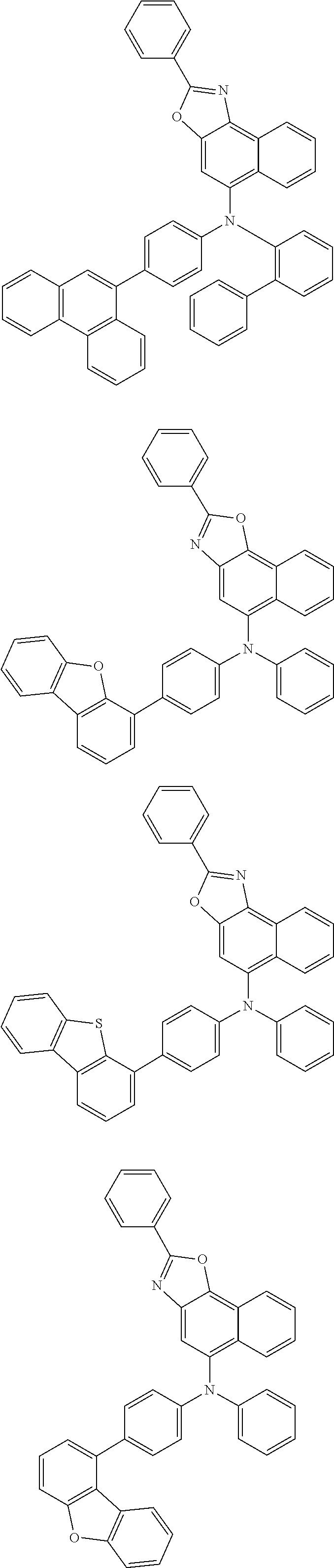 [SMILES: c1ccc(-c2nc3c(cc(N(c4ccc(-c5cc6ccccc6c6ccccc56)cc4)c4ccccc4-c4ccccc4)c4ccccc43)o2)cc1 ; c1ccc(-c2nc3cc(N(c4ccccc4)c4ccc(-c5cccc6c5oc5ccccc56)cc4)c4ccccc4c3o2)cc1 ; c1ccc(-c2nc3c(cc(N(c4ccccc4)c4ccc(-c5cccc6c5sc5ccccc56)cc4)c4ccccc43)o2)cc1 ; c1ccc(-c2nc3cc(N(c4ccccc4)c4ccc(-c5cccc6oc7ccccc7c56)cc4)c4ccccc4c3o2)cc1]

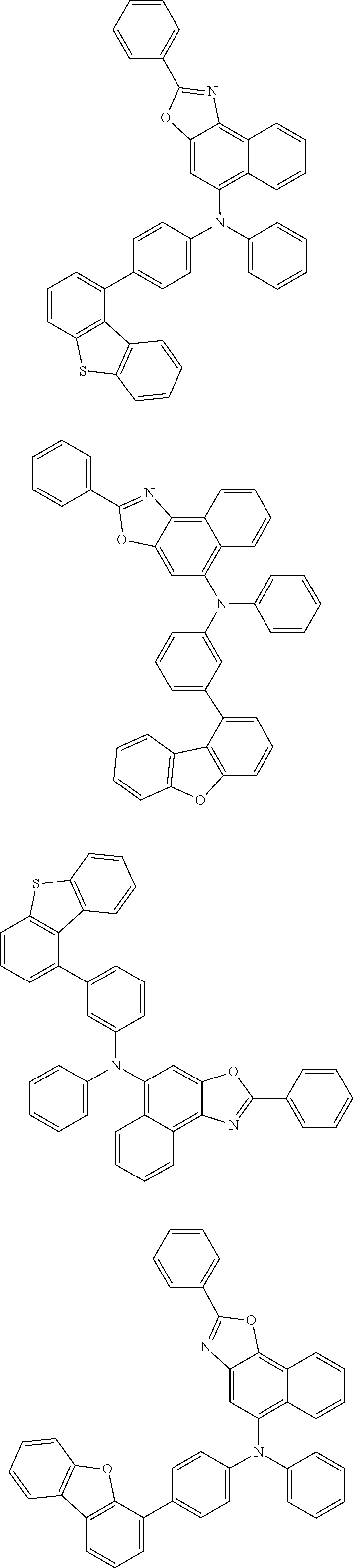 [SMILES: c1ccc(-c2nc3c(cc(N(c4ccccc4)c4ccc(-c5cccc6sc7ccccc7c56)cc4)c4ccccc43)o2)cc1 ; c1ccc(-c2nc3c(cc(N(c4ccccc4)c4cccc(-c5cccc6oc7ccccc7c56)c4)c4ccccc43)o2)cc1 ; c1ccc(-c2nc3c(cc(N(c4ccccc4)c4cccc(-c5cccc6sc7ccccc7c56)c4)c4ccccc43)o2)cc1 ; c1ccc(-c2nc3cc(N(c4ccccc4)c4ccc(-c5cccc6c5oc5ccccc56)cc4)c4ccccc4c3o2)cc1]

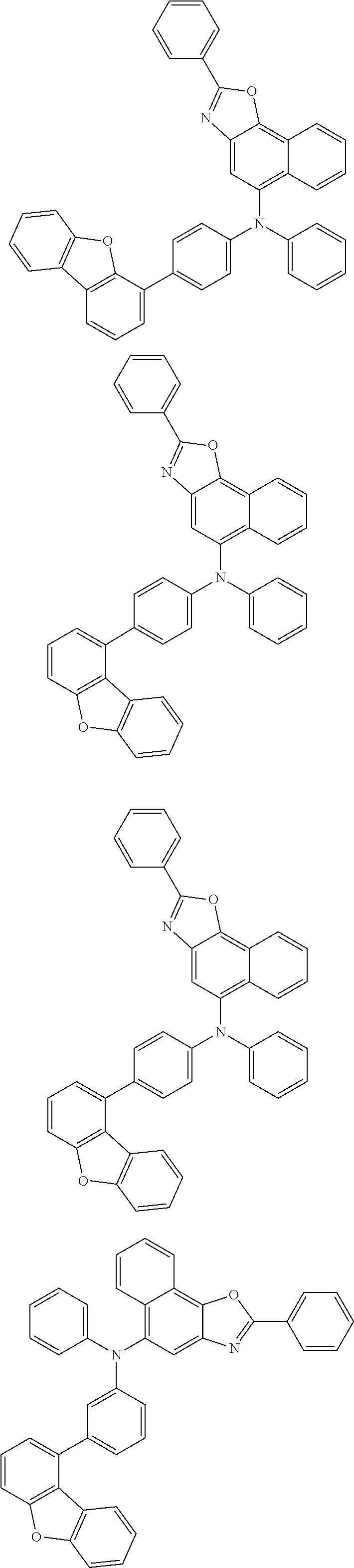

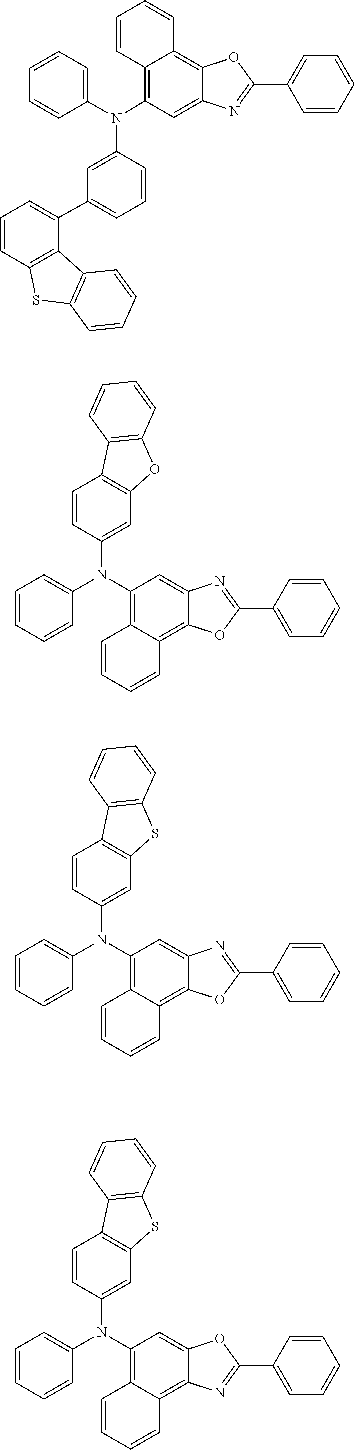 [SMILES: c1ccc(-c2nc3cc(N(c4ccccc4)c4cccc(-c5cccc6sc7ccccc7c56)c4)c4ccccc4c3o2)cc1 ; c1ccc(-c2nc3cc(N(c4ccccc4)c4ccc5c(c4)oc4ccccc45)c4ccccc4c3o2)cc1 ; c1ccc(-c2nc3cc(N(c4ccccc4)c4ccc5c(c4)sc4ccccc45)c4ccccc4c3o2)cc1 ; c1ccc(-c2nc3c(cc(N(c4ccccc4)c4ccc5c(c4)sc4ccccc45)c4ccccc43)o2)cc1]

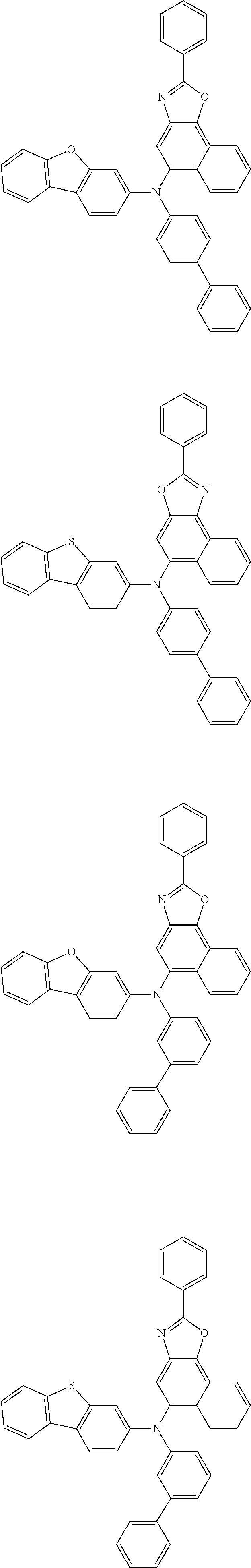 [SMILES: c1ccc(-c2ccc(N(c3ccc4c(c3)oc3ccccc34)c3cc4nc(-c5ccccc5)oc4c4ccccc34)cc2)cc1 ; c1ccc(-c2ccc(N(c3ccc4c(c3)sc3ccccc34)c3cc4oc(-c5ccccc5)nc4c4ccccc34)cc2)cc1 ; c1ccc(-c2cccc(N(c3ccc4c(c3)oc3ccccc34)c3cc4nc(-c5ccccc5)oc4c4ccccc34)c2)cc1 ; c1ccc(-c2cccc(N(c3ccc4c(c3)sc3ccccc34)c3cc4nc(-c5ccccc5)oc4c4ccccc34)c2)cc1]

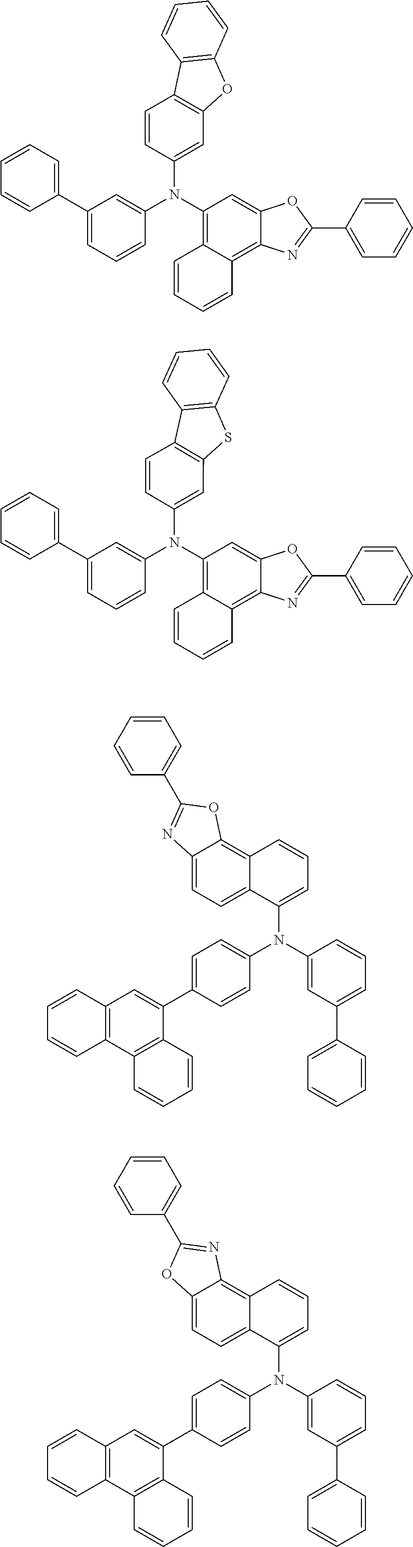

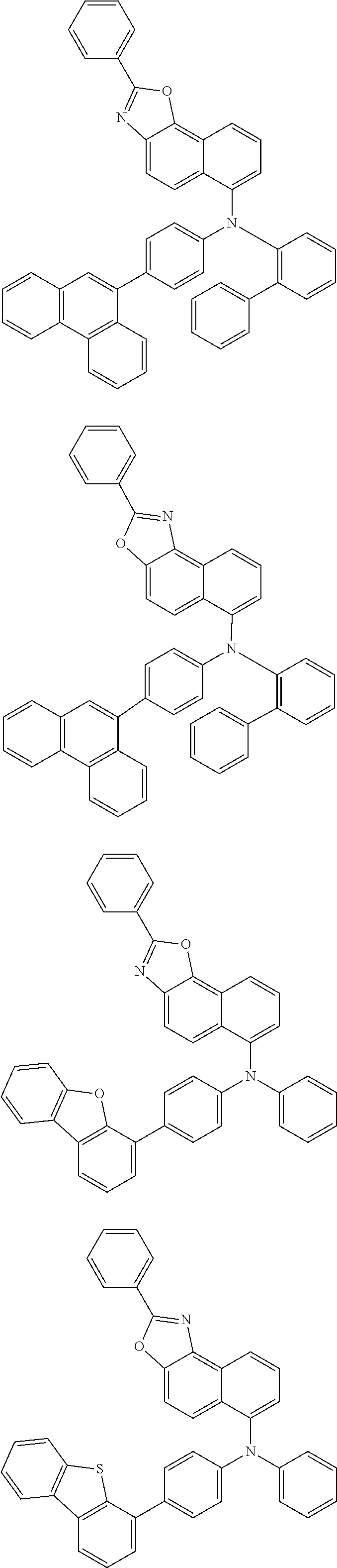

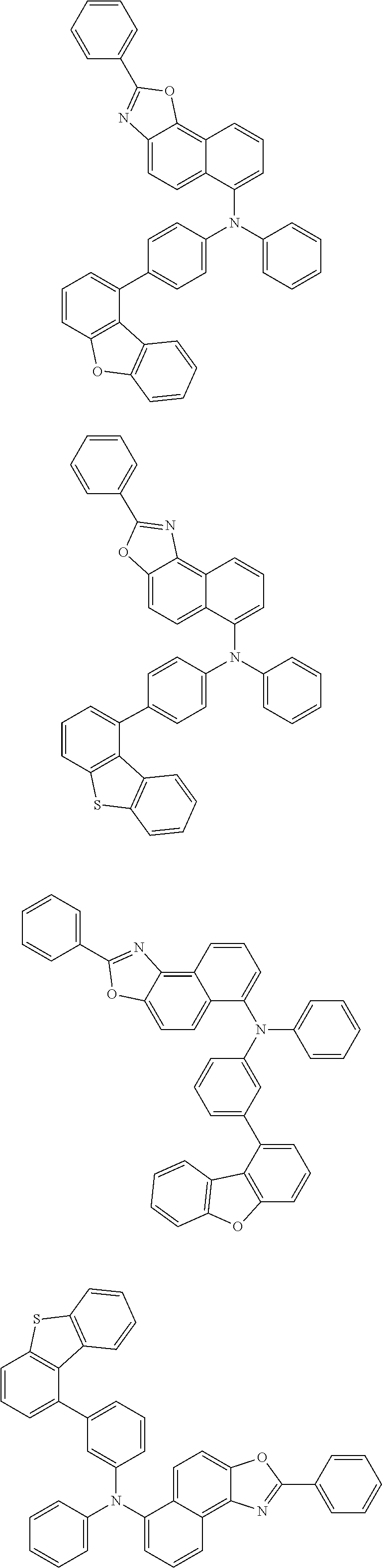

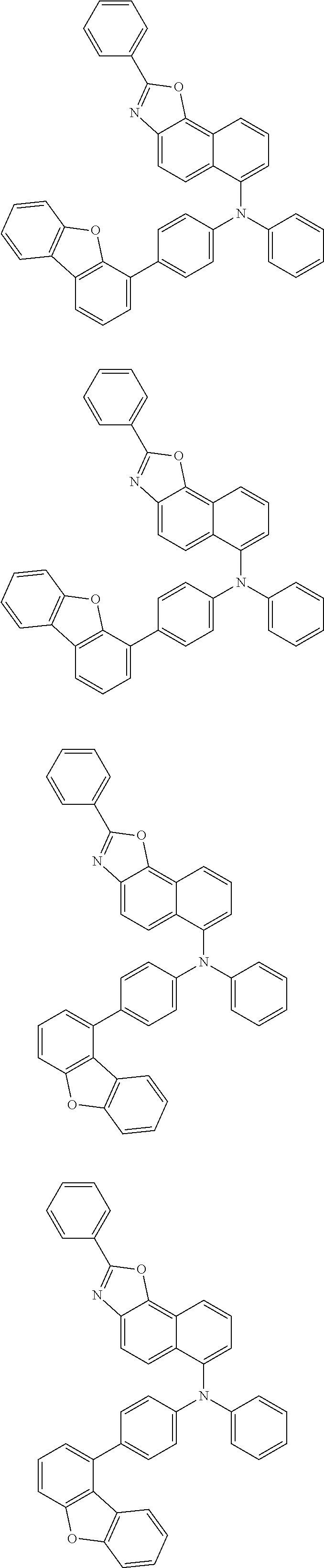

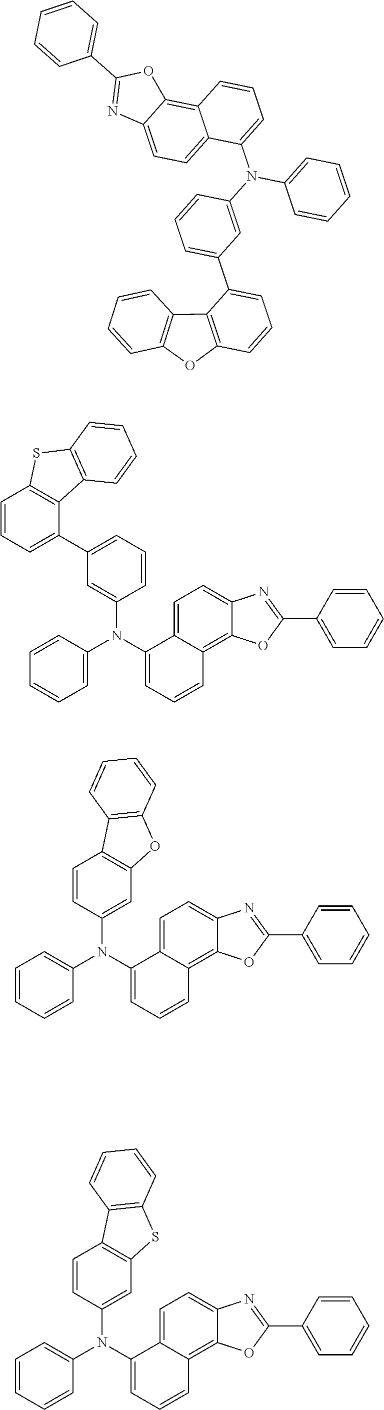 [SMILES: c1ccc(-c2nc3ccc4c(N(c5ccccc5)c5cccc(-c6cccc7oc8ccccc8c67)c5)cccc4c3o2)cc1 ; c1ccc(-c2nc3ccc4c(N(c5ccccc5)c5cccc(-c6cccc7sc8ccccc8c67)c5)cccc4c3o2)cc1 ; c1ccc(-c2nc3ccc4c(N(c5ccccc5)c5ccc6c(c5)oc5ccccc56)cccc4c3o2)cc1 ; c1ccc(-c2nc3ccc4c(N(c5ccccc5)c5ccc6c(c5)sc5ccccc56)cccc4c3o2)cc1]

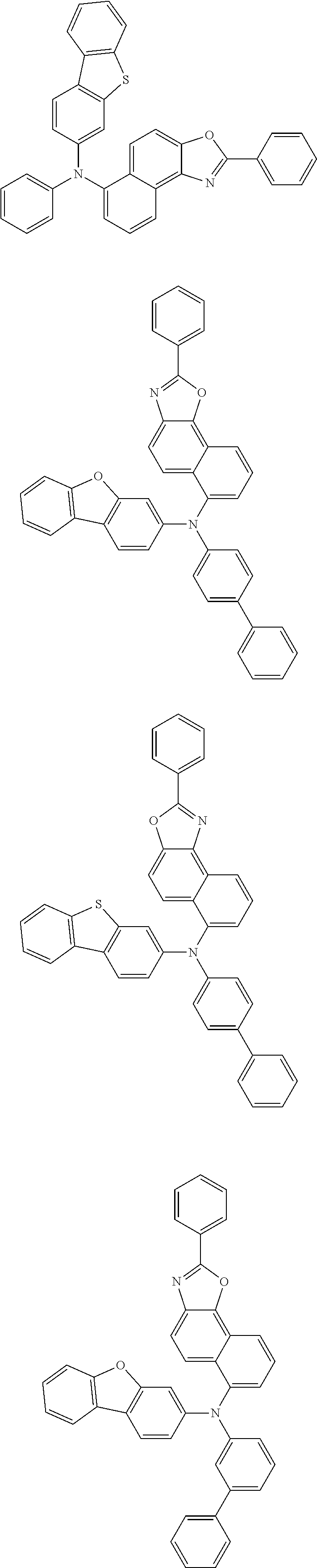 [SMILES: c1ccc(-c2nc3c(ccc4c(N(c5ccccc5)c5ccc6c(c5)sc5ccccc56)cccc43)o2)cc1 ; c1ccc(-c2ccc(N(c3ccc4c(c3)oc3ccccc34)c3cccc4c3ccc3nc(-c5ccccc5)oc34)cc2)cc1 ; c1ccc(-c2ccc(N(c3ccc4c(c3)sc3ccccc34)c3cccc4c3ccc3oc(-c5ccccc5)nc34)cc2)cc1 ; c1ccc(-c2cccc(N(c3ccc4c(c3)oc3ccccc34)c3cccc4c3ccc3nc(-c5ccccc5)oc34)c2)cc1]

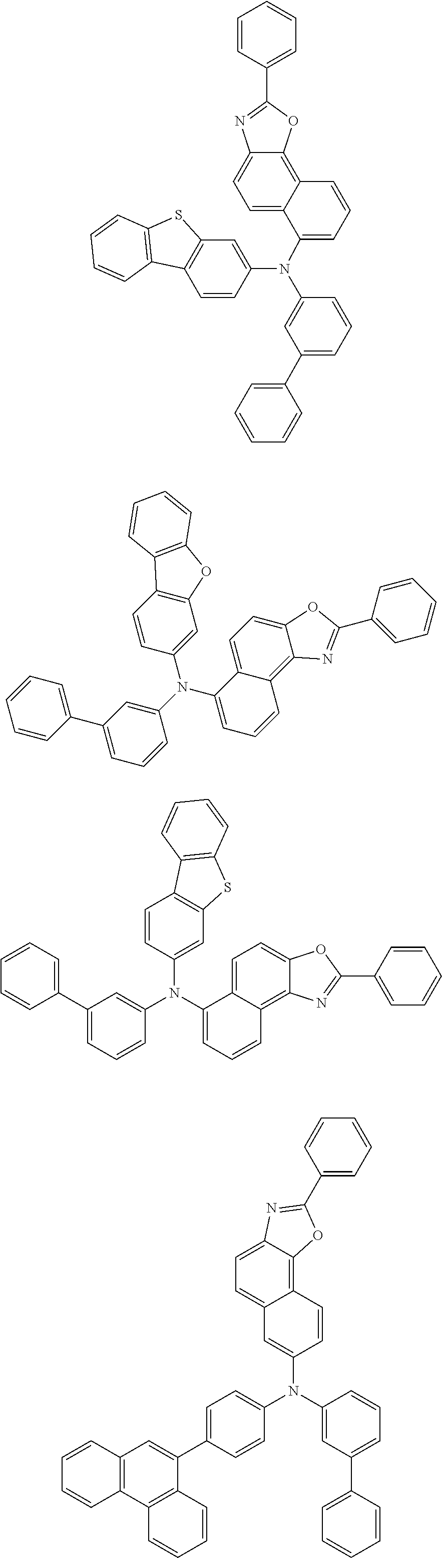 [SMILES: c1ccc(-c2cccc(N(c3ccc4c(c3)sc3ccccc34)c3cccc4c3ccc3nc(-c5ccccc5)oc34)c2)cc1 ; c1ccc(-c2cccc(N(c3ccc4c(c3)oc3ccccc34)c3cccc4c3ccc3oc(-c5ccccc5)nc34)c2)cc1 ; c1ccc(-c2cccc(N(c3ccc4c(c3)sc3ccccc34)c3cccc4c3ccc3oc(-c5ccccc5)nc34)c2)cc1 ; c1ccc(-c2cccc(N(c3ccc(-c4cc5ccccc5c5ccccc45)cc3)c3ccc4c(ccc5nc(-c6ccccc6)oc54)c3)c2)cc1]

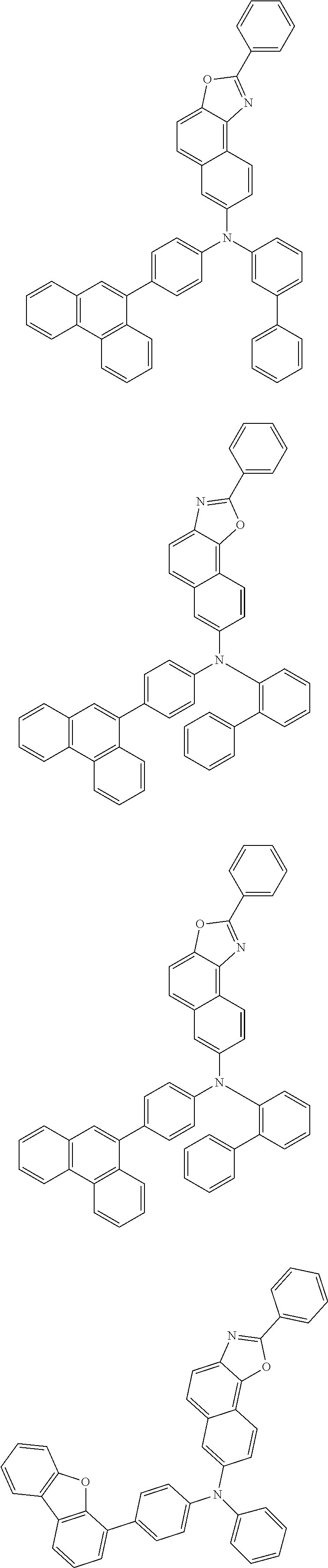 [SMILES: c1ccc(-c2cccc(N(c3ccc(-c4cc5ccccc5c5ccccc45)cc3)c3ccc4c(ccc5oc(-c6ccccc6)nc54)c3)c2)cc1 ; c1ccc(-c2nc3ccc4cc(N(c5ccc(-c6cc7ccccc7c7ccccc67)cc5)c5ccccc5-c5ccccc5)ccc4c3o2)cc1 ; c1ccc(-c2nc3c(ccc4cc(N(c5ccc(-c6cc7ccccc7c7ccccc67)cc5)c5ccccc5-c5ccccc5)ccc43)o2)cc1 ; c1ccc(-c2nc3ccc4cc(N(c5ccccc5)c5ccc(-c6cccc7c6oc6ccccc67)cc5)ccc4c3o2)cc1]

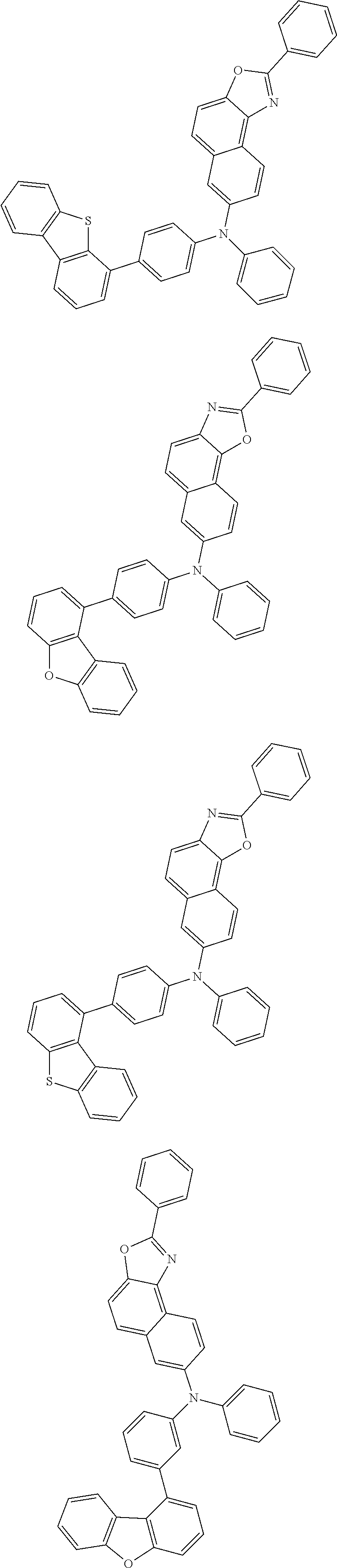

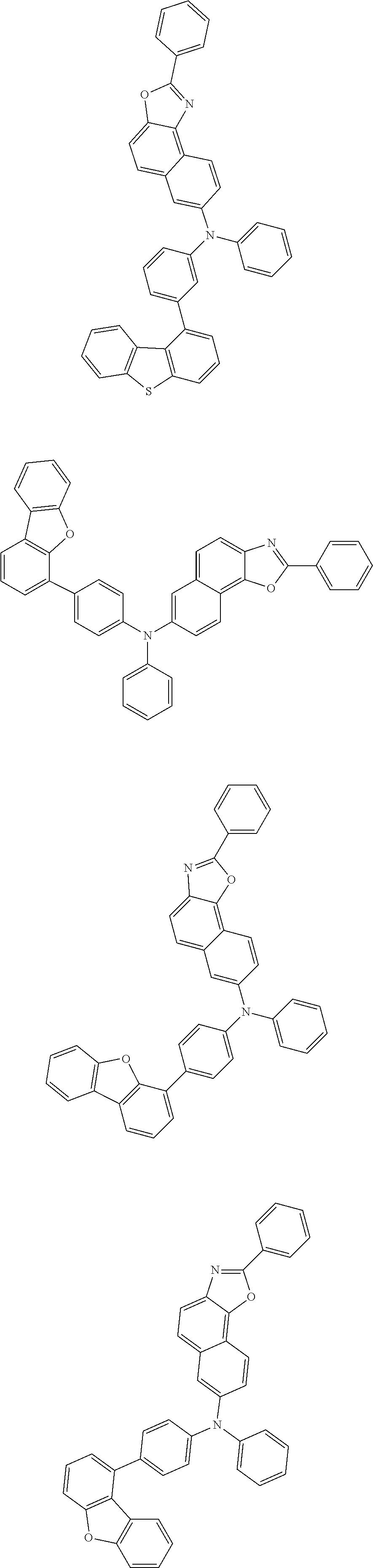 [SMILES: c1ccc(-c2nc3c(ccc4cc(N(c5ccccc5)c5cccc(-c6cccc7sc8ccccc8c67)c5)ccc43)o2)cc1 ; c1ccc(-c2nc3ccc4cc(N(c5ccccc5)c5ccc(-c6cccc7c6oc6ccccc67)cc5)ccc4c3o2)cc1 ; c1ccc(-c2nc3ccc4cc(N(c5ccccc5)c5ccc(-c6cccc7c6oc6ccccc67)cc5)ccc4c3o2)cc1 ; c1ccc(-c2nc3ccc4cc(N(c5ccccc5)c5ccc(-c6cccc7oc8ccccc8c67)cc5)ccc4c3o2)cc1]

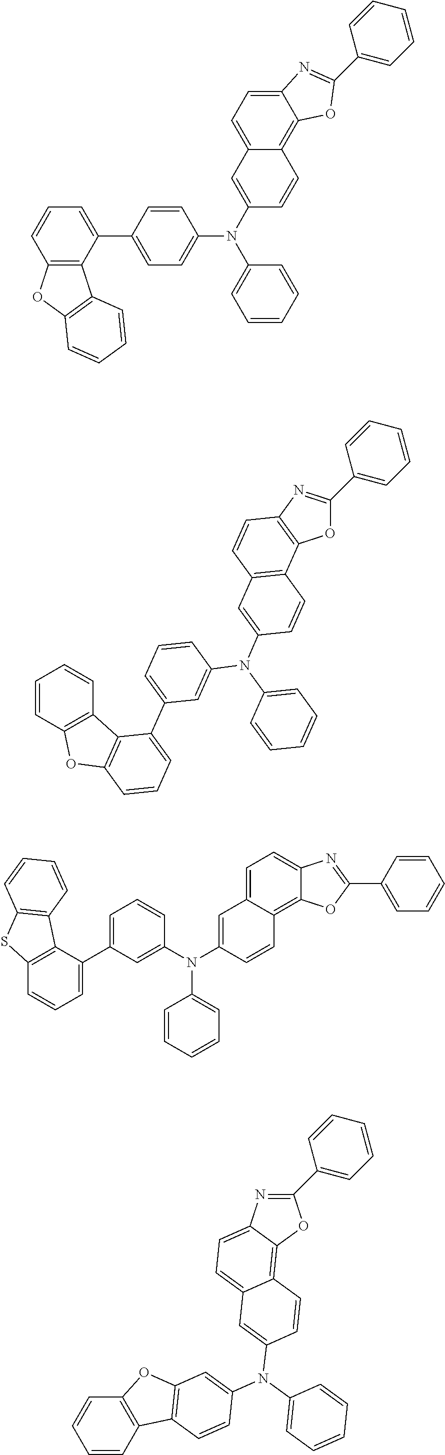 [SMILES: c1ccc(-c2nc3ccc4cc(N(c5ccccc5)c5ccc(-c6cccc7oc8ccccc8c67)cc5)ccc4c3o2)cc1 ; c1ccc(-c2nc3ccc4cc(N(c5ccccc5)c5cccc(-c6cccc7oc8ccccc8c67)c5)ccc4c3o2)cc1 ; c1ccc(-c2nc3ccc4cc(N(c5ccccc5)c5cccc(-c6cccc7sc8ccccc8c67)c5)ccc4c3o2)cc1 ; c1ccc(-c2nc3ccc4cc(N(c5ccccc5)c5ccc6c(c5)oc5ccccc56)ccc4c3o2)cc1]

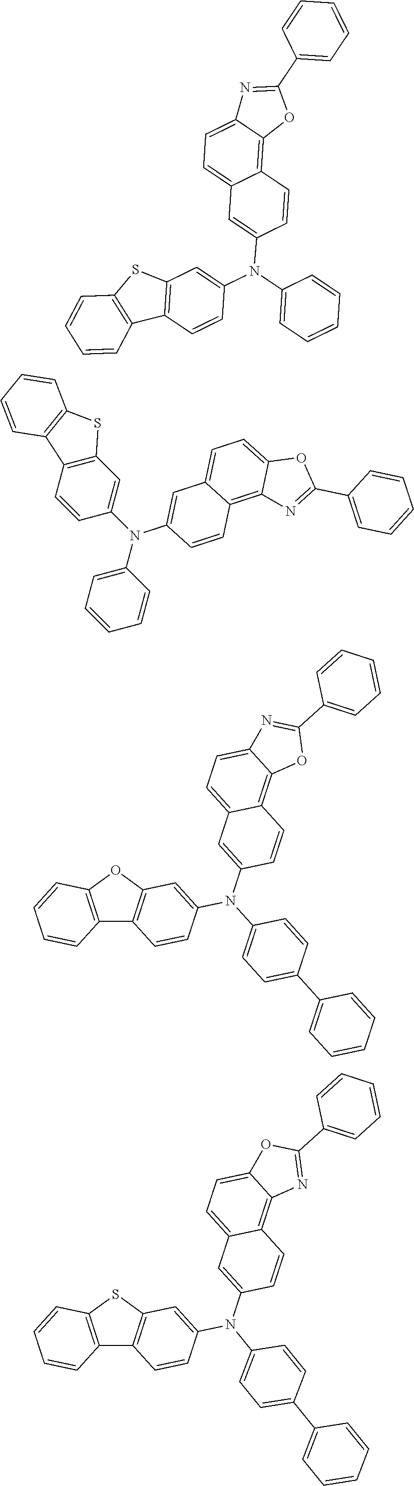

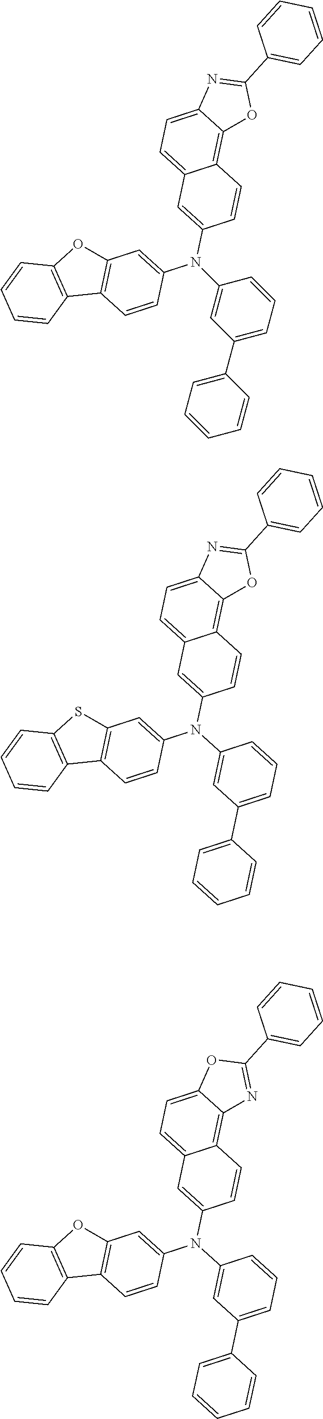 [SMILES: c1ccc(-c2cccc(N(c3ccc4c(ccc5nc(-c6ccccc6)oc54)c3)c3ccc4c(c3)oc3ccccc34)c2)cc1 ; c1ccc(-c2cccc(N(c3ccc4c(ccc5nc(-c6ccccc6)oc54)c3)c3ccc4c(c3)sc3ccccc34)c2)cc1 ; c1ccc(-c2cccc(N(c3ccc4c(ccc5oc(-c6ccccc6)nc54)c3)c3ccc4c(c3)oc3ccccc34)c2)cc1]

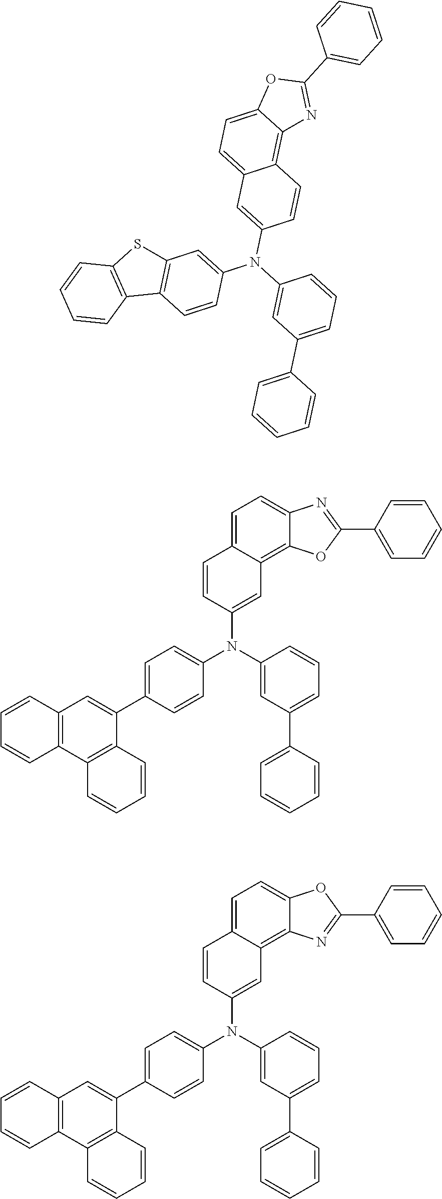 [SMILES: c1ccc(-c2cccc(N(c3ccc4c(ccc5oc(-c6ccccc6)nc54)c3)c3ccc4c(c3)sc3ccccc34)c2)cc1 ; c1ccc(-c2cccc(N(c3ccc(-c4cc5ccccc5c5ccccc45)cc3)c3ccc4ccc5nc(-c6ccccc6)oc5c4c3)c2)cc1 ; c1ccc(-c2cccc(N(c3ccc(-c4cc5ccccc5c5ccccc45)cc3)c3ccc4ccc5oc(-c6ccccc6)nc5c4c3)c2)cc1]

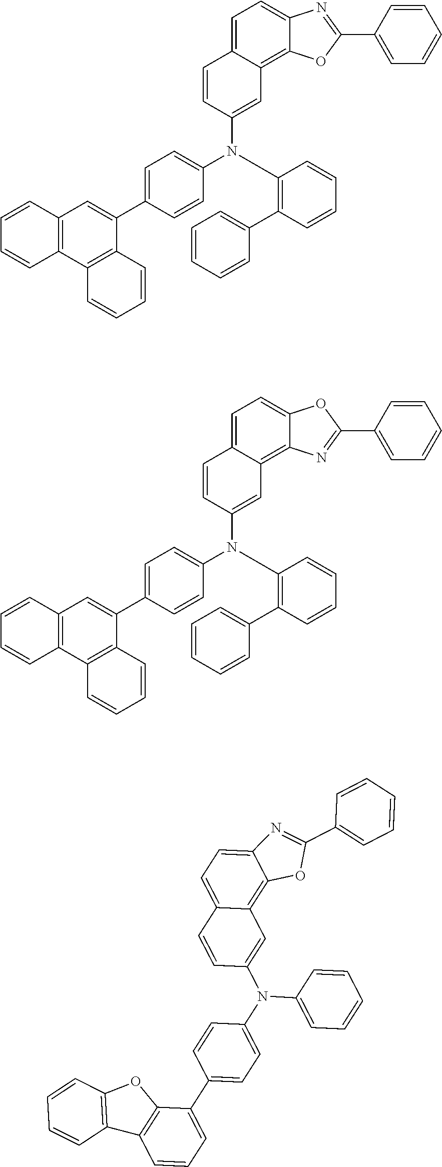 [SMILES: c1ccc(-c2nc3ccc4ccc(N(c5ccc(-c6cc7ccccc7c7ccccc67)cc5)c5ccccc5-c5ccccc5)cc4c3o2)cc1 ; c1ccc(-c2nc3c(ccc4ccc(N(c5ccc(-c6cc7ccccc7c7ccccc67)cc5)c5ccccc5-c5ccccc5)cc43)o2)cc1 ; c1ccc(-c2nc3ccc4ccc(N(c5ccccc5)c5ccc(-c6cccc7c6oc6ccccc67)cc5)cc4c3o2)cc1]

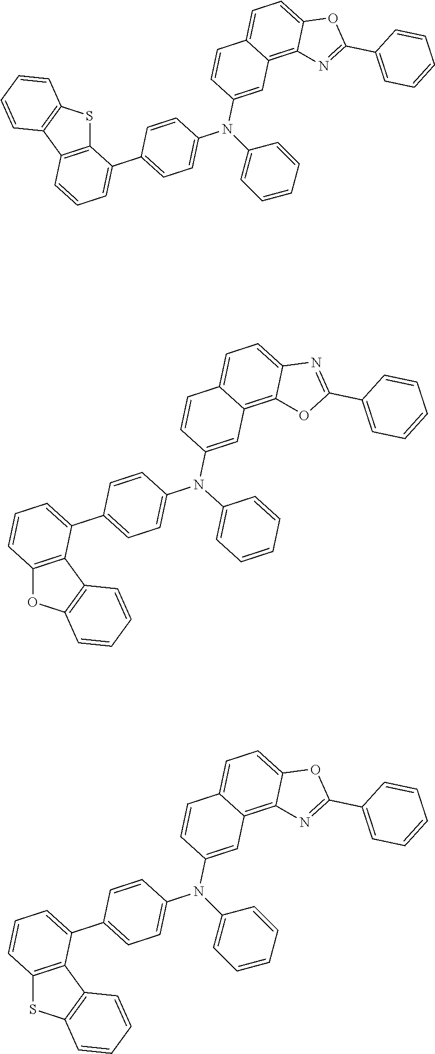 [SMILES: c1ccc(-c2nc3c(ccc4ccc(N(c5ccccc5)c5ccc(-c6cccc7c6sc6ccccc67)cc5)cc43)o2)cc1 ; c1ccc(-c2nc3ccc4ccc(N(c5ccccc5)c5ccc(-c6cccc7oc8ccccc8c67)cc5)cc4c3o2)cc1 ; c1ccc(-c2nc3c(ccc4ccc(N(c5ccccc5)c5ccc(-c6cccc7sc8ccccc8c67)cc5)cc43)o2)cc1]

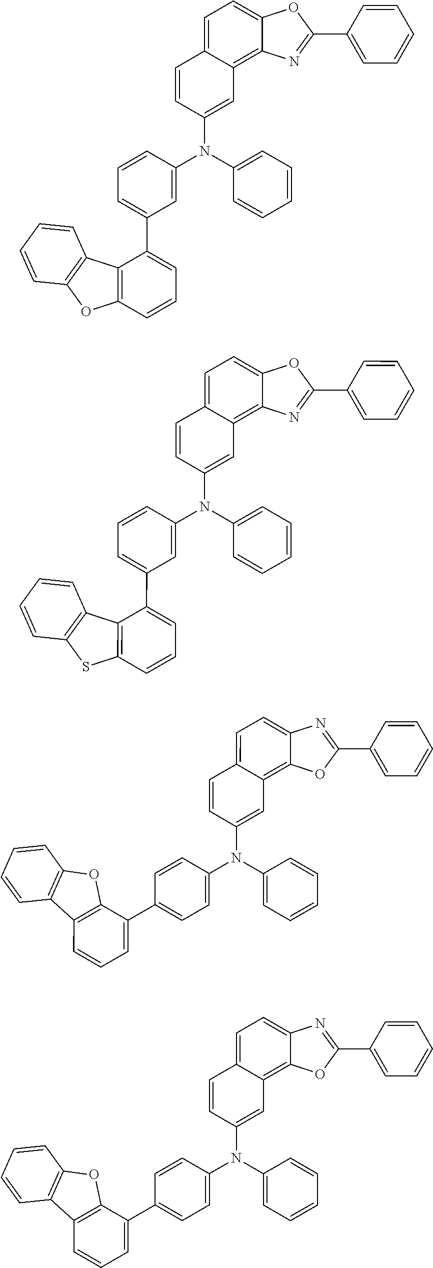 [SMILES: c1ccc(-c2nc3c(ccc4ccc(N(c5ccccc5)c5cccc(-c6cccc7oc8ccccc8c67)c5)cc43)o2)cc1 ; c1ccc(-c2nc3c(ccc4ccc(N(c5ccccc5)c5cccc(-c6cccc7sc8ccccc8c67)c5)cc43)o2)cc1 ; c1ccc(-c2nc3ccc4ccc(N(c5ccccc5)c5ccc(-c6cccc7c6oc6ccccc67)cc5)cc4c3o2)cc1 ; c1ccc(-c2nc3ccc4ccc(N(c5ccccc5)c5ccc(-c6cccc7c6oc6ccccc67)cc5)cc4c3o2)cc1]

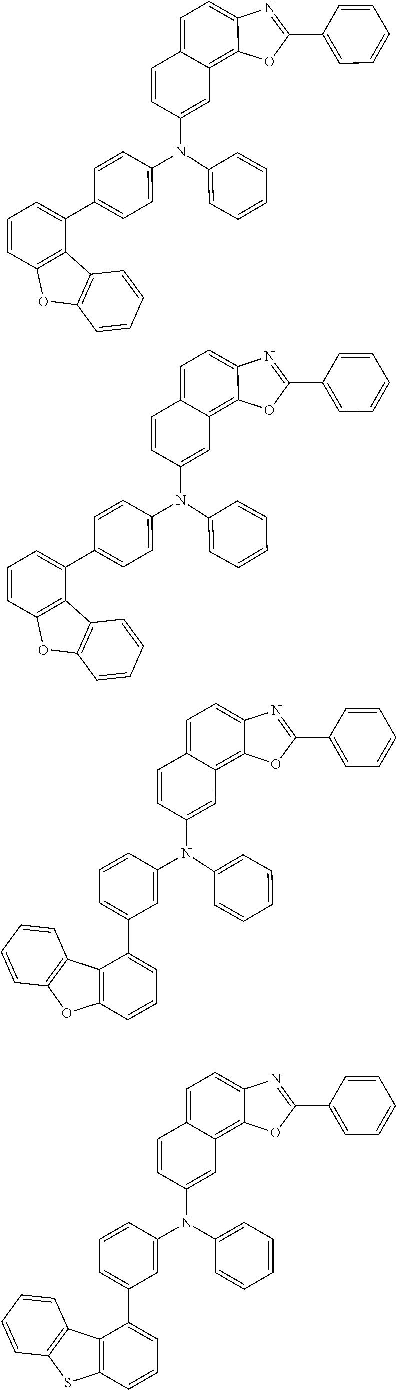 [SMILES: c1ccc(-c2nc3ccc4ccc(N(c5ccccc5)c5ccc(-c6cccc7oc8ccccc8c67)cc5)cc4c3o2)cc1 ; c1ccc(-c2nc3ccc4ccc(N(c5ccccc5)c5ccc(-c6cccc7oc8ccccc8c67)cc5)cc4c3o2)cc1 ; c1ccc(-c2nc3ccc4ccc(N(c5ccccc5)c5cccc(-c6cccc7oc8ccccc8c67)c5)cc4c3o2)cc1 ; c1ccc(-c2nc3ccc4ccc(N(c5ccccc5)c5cccc(-c6cccc7sc8ccccc8c67)c5)cc4c3o2)cc1]

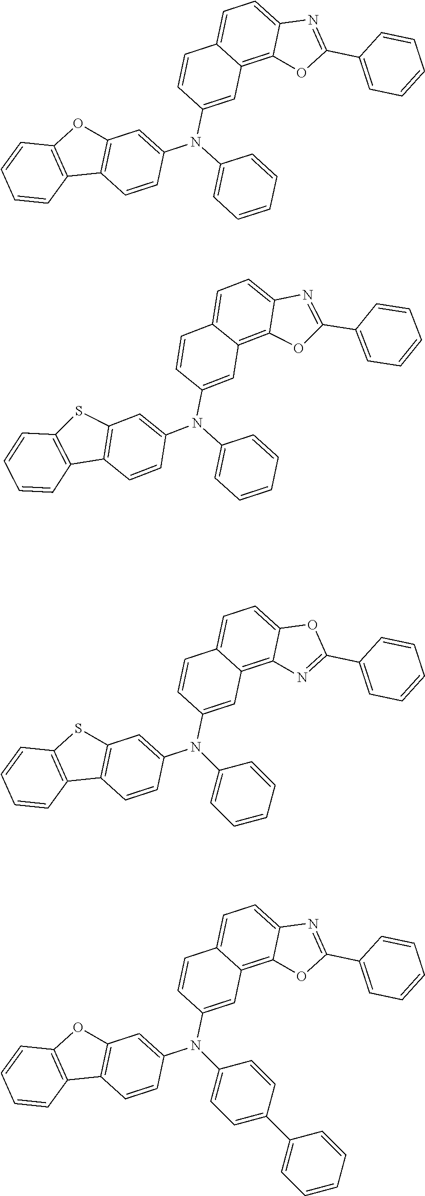 [SMILES: c1ccc(-c2nc3ccc4ccc(N(c5ccccc5)c5ccc6c(c5)oc5ccccc56)cc4c3o2)cc1 ; c1ccc(-c2nc3ccc4ccc(N(c5ccccc5)c5ccc6c(c5)sc5ccccc56)cc4c3o2)cc1 ; c1ccc(-c2nc3c(ccc4ccc(N(c5ccccc5)c5ccc6c(c5)sc5ccccc56)cc43)o2)cc1 ; c1ccc(-c2ccc(N(c3ccc4c(c3)oc3ccccc34)c3ccc4ccc5nc(-c6ccccc6)oc5c4c3)cc2)cc1]

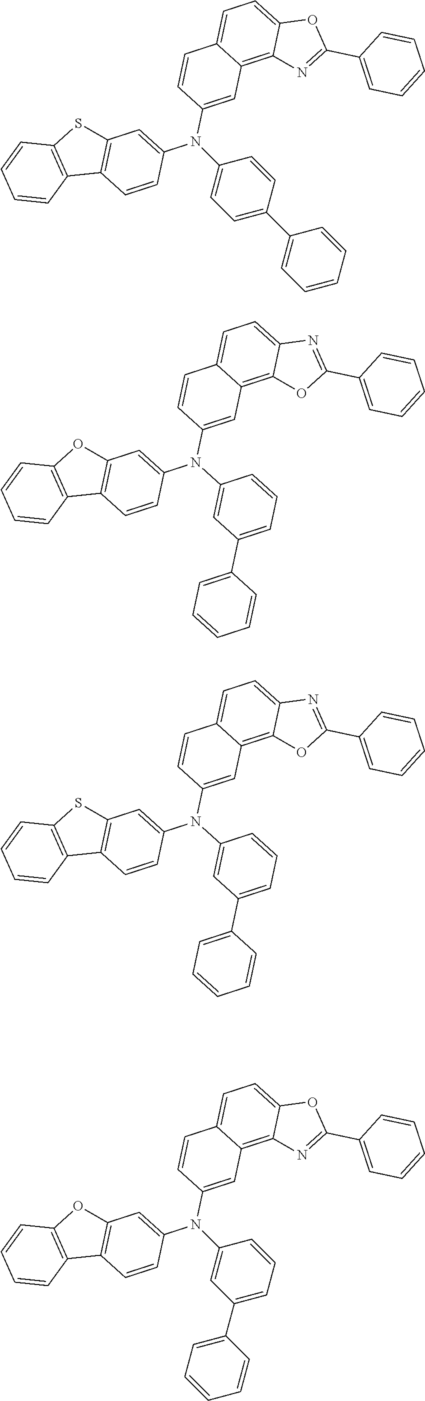 [SMILES: c1ccc(-c2ccc(N(c3ccc4c(c3)sc3ccccc34)c3ccc4ccc5oc(-c6ccccc6)nc5c4c3)cc2)cc1 ; c1ccc(-c2cccc(N(c3ccc4c(c3)oc3ccccc34)c3ccc4ccc5nc(-c6ccccc6)oc5c4c3)c2)cc1 ; c1ccc(-c2cccc(N(c3ccc4c(c3)sc3ccccc34)c3ccc4ccc5nc(-c6ccccc6)oc5c4c3)c2)cc1 ; c1ccc(-c2cccc(N(c3ccc4c(c3)oc3ccccc34)c3ccc4ccc5oc(-c6ccccc6)nc5c4c3)c2)cc1]

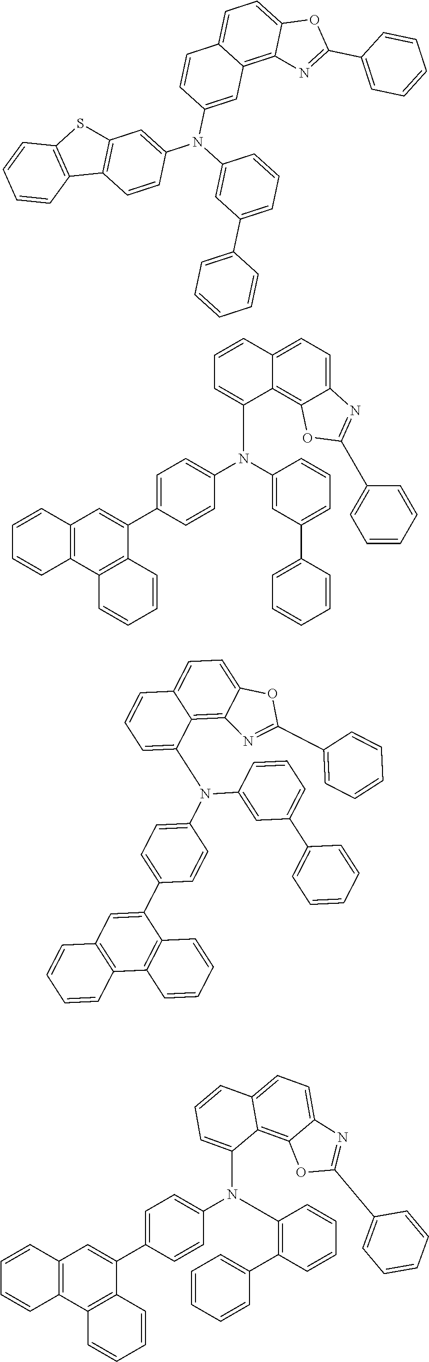

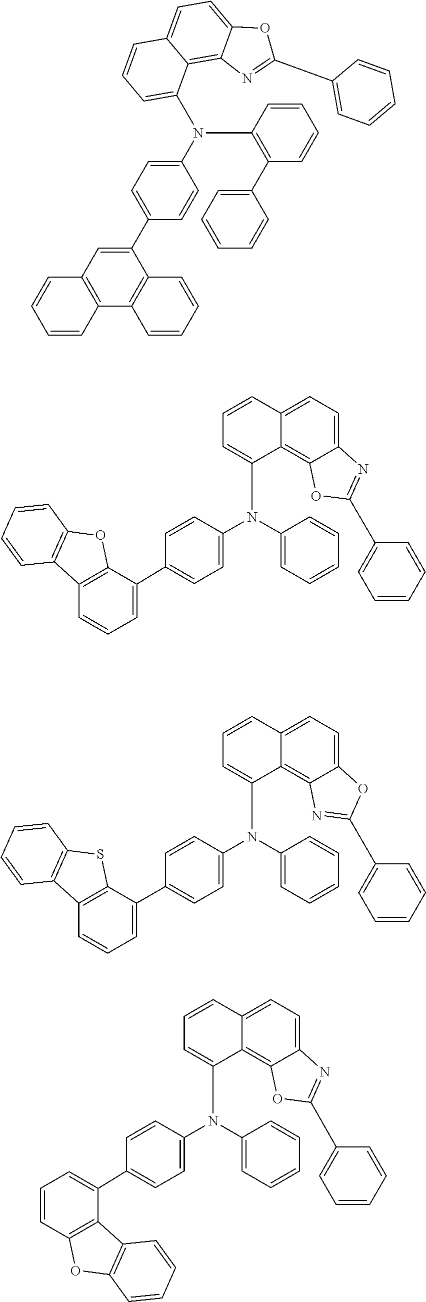 [SMILES: c1ccc(-c2nc3c(ccc4cccc(N(c5ccc(-c6cc7ccccc7c7ccccc67)cc5)c5ccccc5-c5ccccc5)c43)o2)cc1 ; c1ccc(-c2nc3ccc4cccc(N(c5ccccc5)c5ccc(-c6cccc7c6oc6ccccc67)cc5)c4c3o2)cc1 ; c1ccc(-c2nc3c(ccc4cccc(N(c5ccccc5)c5ccc(-c6cccc7c6sc6ccccc67)cc5)c43)o2)cc1 ; c1ccc(-c2nc3ccc4cccc(N(c5ccccc5)c5ccc(-c6cccc7oc8ccccc8c67)cc5)c4c3o2)cc1]

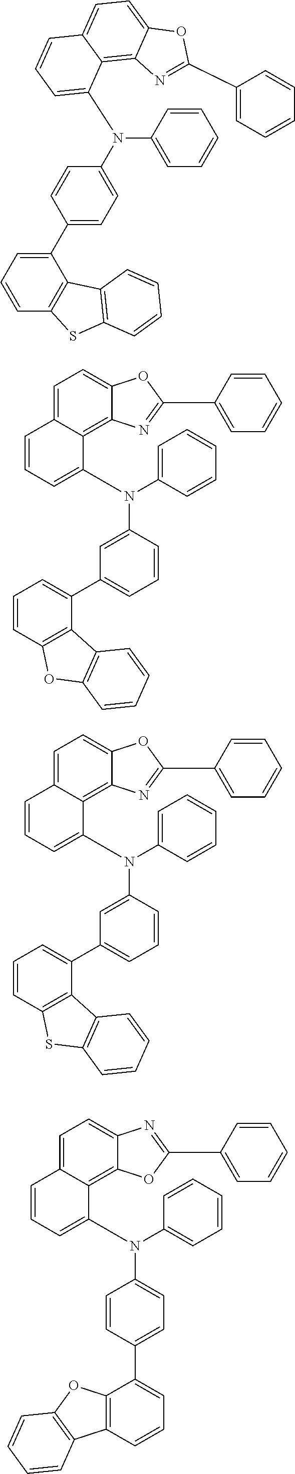

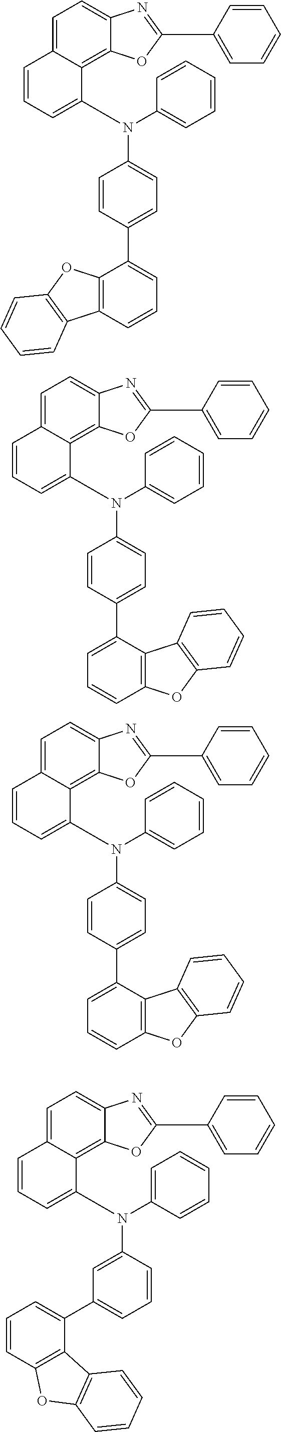

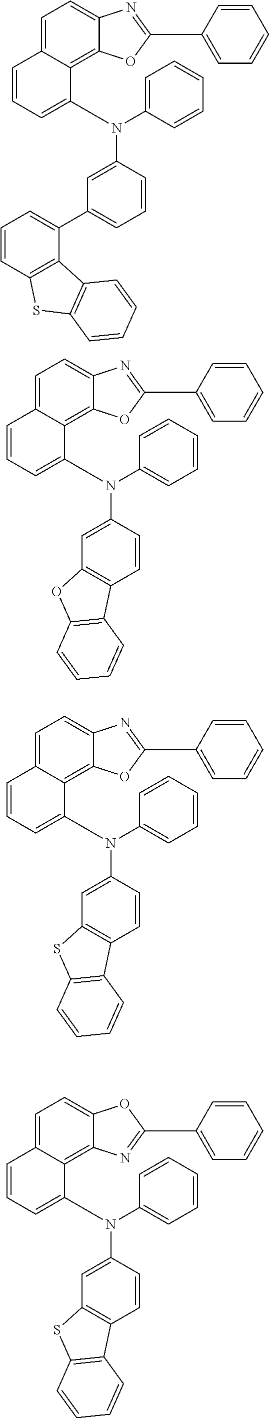

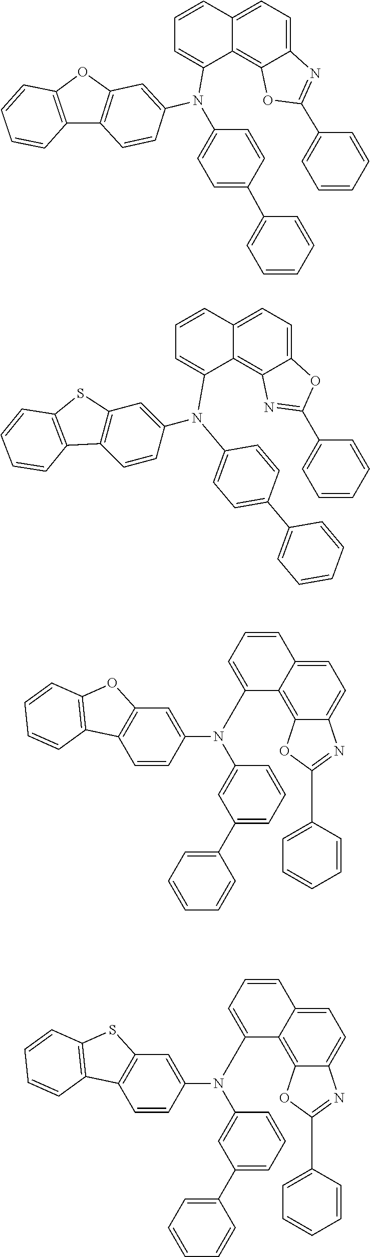

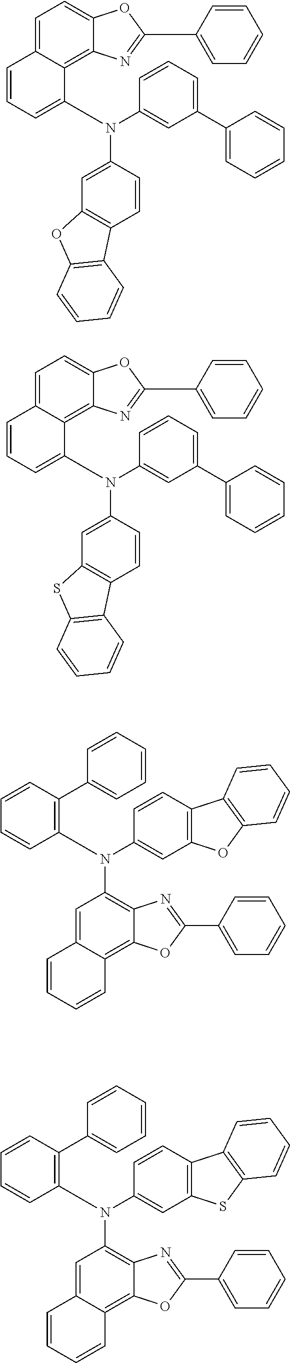

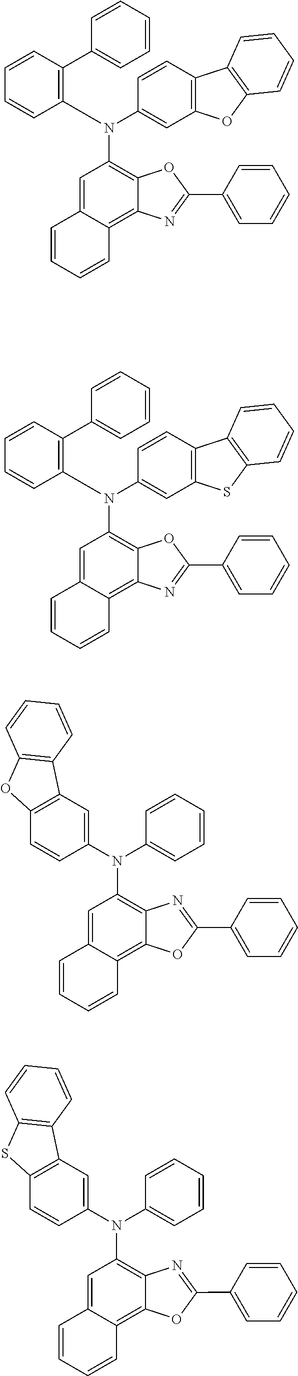 [SMILES: c1ccc(-c2nc3c(o2)c(N(c2ccc4c(c2)oc2ccccc24)c2ccccc2-c2ccccc2)cc2ccccc23)cc1 ; c1ccc(-c2nc3c(o2)c(N(c2ccc4c(c2)sc2ccccc24)c2ccccc2-c2ccccc2)cc2ccccc23)cc1 ; c1ccc(-c2nc3c(N(c4ccccc4)c4ccc5oc6ccccc6c5c4)cc4ccccc4c3o2)cc1 ; c1ccc(-c2nc3c(N(c4ccccc4)c4ccc5sc6ccccc6c5c4)cc4ccccc4c3o2)cc1]

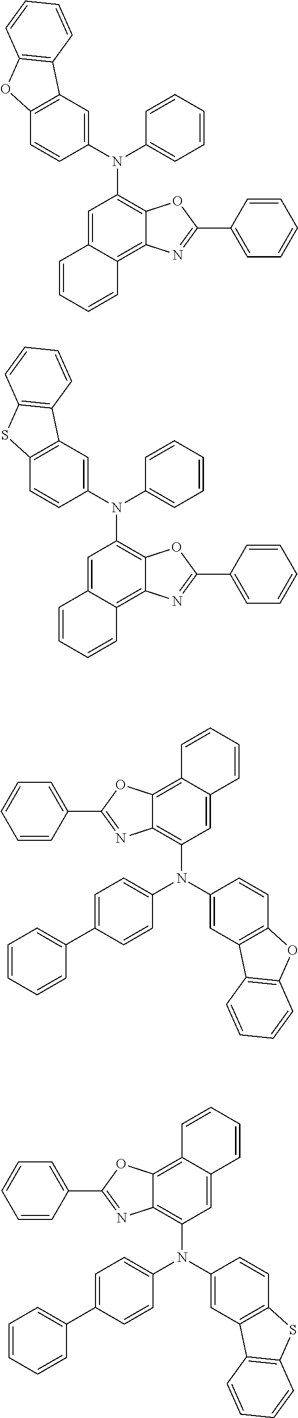 [SMILES: c1ccc(-c2nc3c(o2)c(N(c2ccccc2)c2ccc4oc5ccccc5c4c2)cc2ccccc23)cc1 ; c1ccc(-c2nc3c(o2)c(N(c2ccccc2)c2ccc4sc5ccccc5c4c2)cc2ccccc23)cc1 ; c1ccc(-c2ccc(N(c3ccc4oc5ccccc5c4c3)c3cc4ccccc4c4oc(-c5ccccc5)nc34)cc2)cc1 ; c1ccc(-c2ccc(N(c3ccc4sc5ccccc5c4c3)c3cc4ccccc4c4oc(-c5ccccc5)nc34)cc2)cc1]

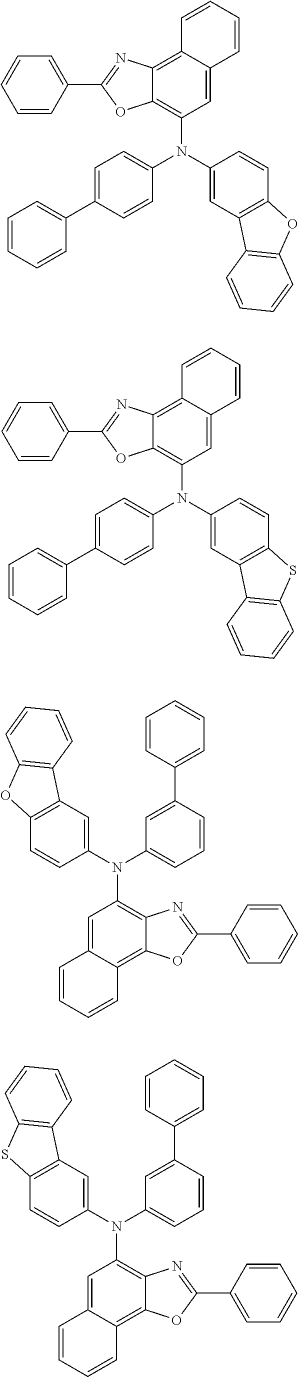 [SMILES: c1ccc(-c2ccc(N(c3ccc4oc5ccccc5c4c3)c3cc4ccccc4c4nc(-c5ccccc5)oc34)cc2)cc1 ; c1ccc(-c2ccc(N(c3ccc4sc5ccccc5c4c3)c3cc4ccccc4c4nc(-c5ccccc5)oc34)cc2)cc1 ; c1ccc(-c2cccc(N(c3ccc4oc5ccccc5c4c3)c3cc4ccccc4c4oc(-c5ccccc5)nc34)c2)cc1 ; c1ccc(-c2cccc(N(c3ccc4sc5ccccc5c4c3)c3cc4ccccc4c4oc(-c5ccccc5)nc34)c2)cc1]

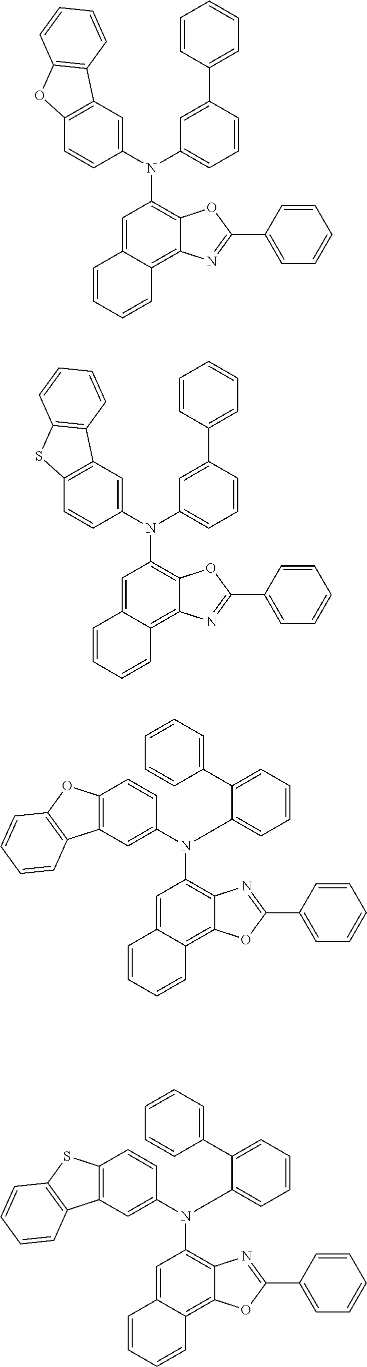 [SMILES: c1ccc(-c2cccc(N(c3ccc4oc5ccccc5c4c3)c3cc4ccccc4c4nc(-c5ccccc5)oc34)c2)cc1 ; c1ccc(-c2cccc(N(c3ccc4sc5ccccc5c4c3)c3cc4ccccc4c4nc(-c5ccccc5)oc34)c2)cc1 ; c1ccc(-c2nc3c(N(c4ccc5oc6ccccc6c5c4)c4ccccc4-c4ccccc4)cc4ccccc4c3o2)cc1 ; c1ccc(-c2nc3c(N(c4ccc5sc6ccccc6c5c4)c4ccccc4-c4ccccc4)cc4ccccc4c3o2)cc1]

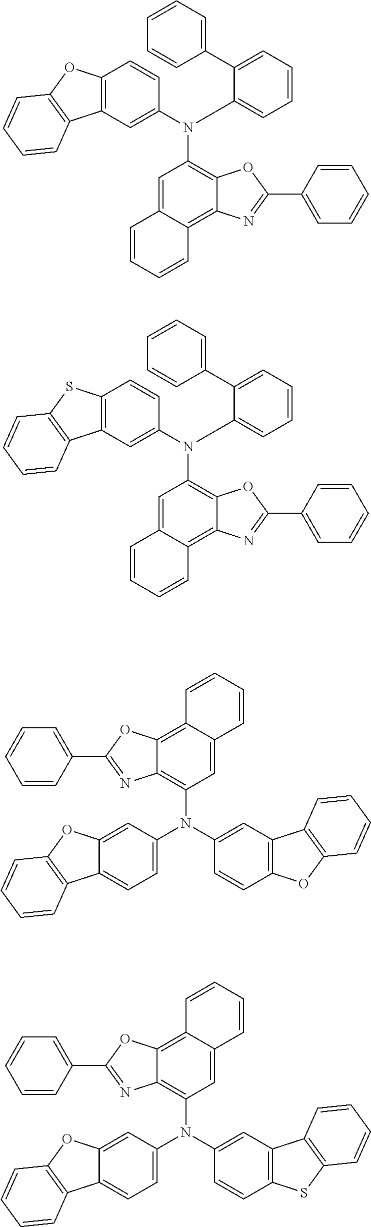

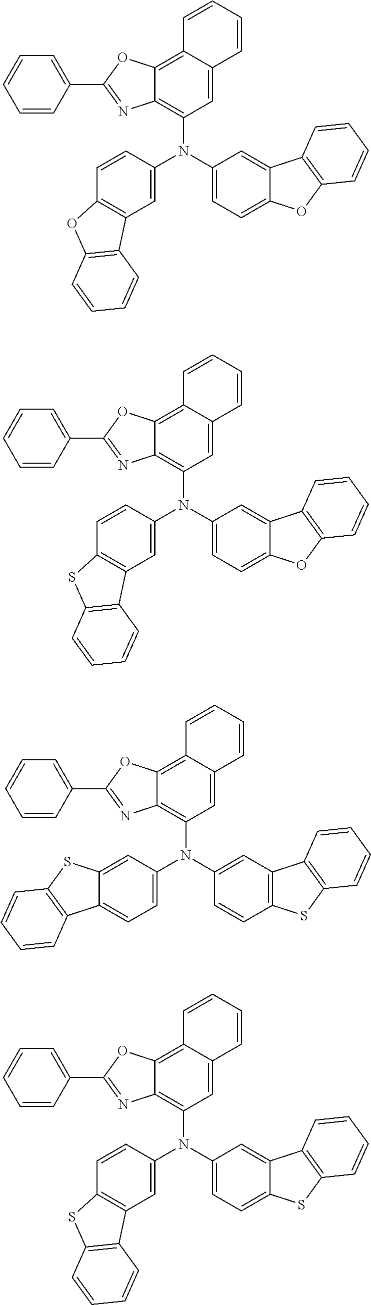 [SMILES: c1ccc(-c2nc3c(N(c4ccc5oc6ccccc6c5c4)c4ccc5oc6ccccc6c5c4)cc4ccccc4c3o2)cc1 ; c1ccc(-c2nc3c(N(c4ccc5oc6ccccc6c5c4)c4ccc5sc6ccccc6c5c4)cc4ccccc4c3o2)cc1 ; c1ccc(-c2nc3c(N(c4ccc5c(c4)sc4ccccc45)c4ccc5sc6ccccc6c5c4)cc4ccccc4c3o2)cc1 ; c1ccc(-c2nc3c(N(c4ccc5sc6ccccc6c5c4)c4ccc5sc6ccccc6c5c4)cc4ccccc4c3o2)cc1]

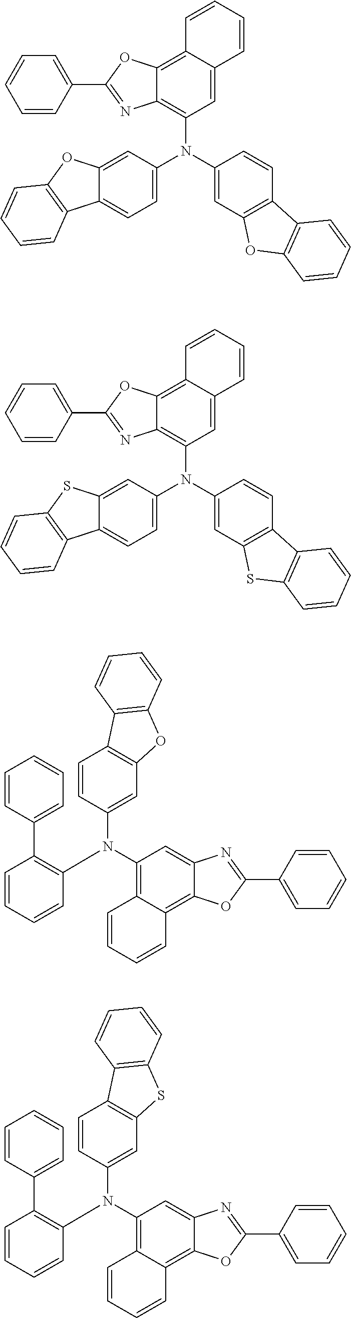

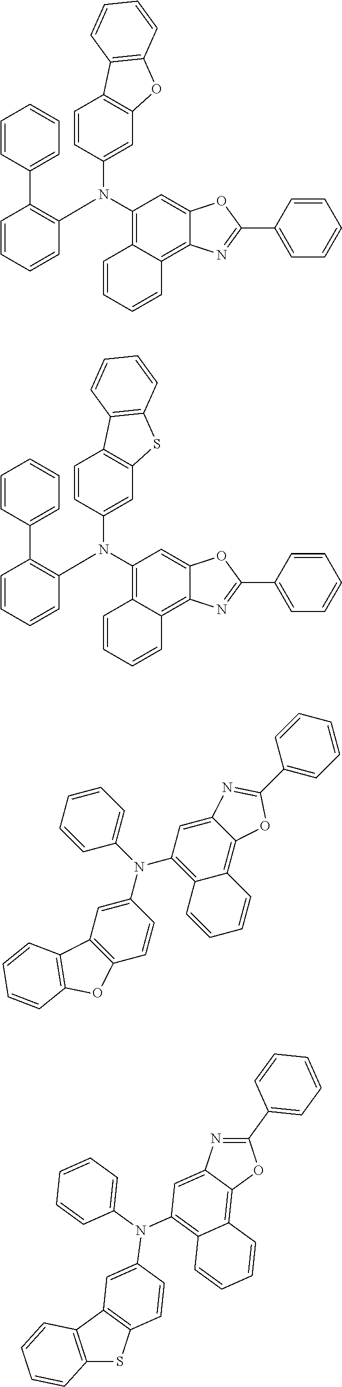

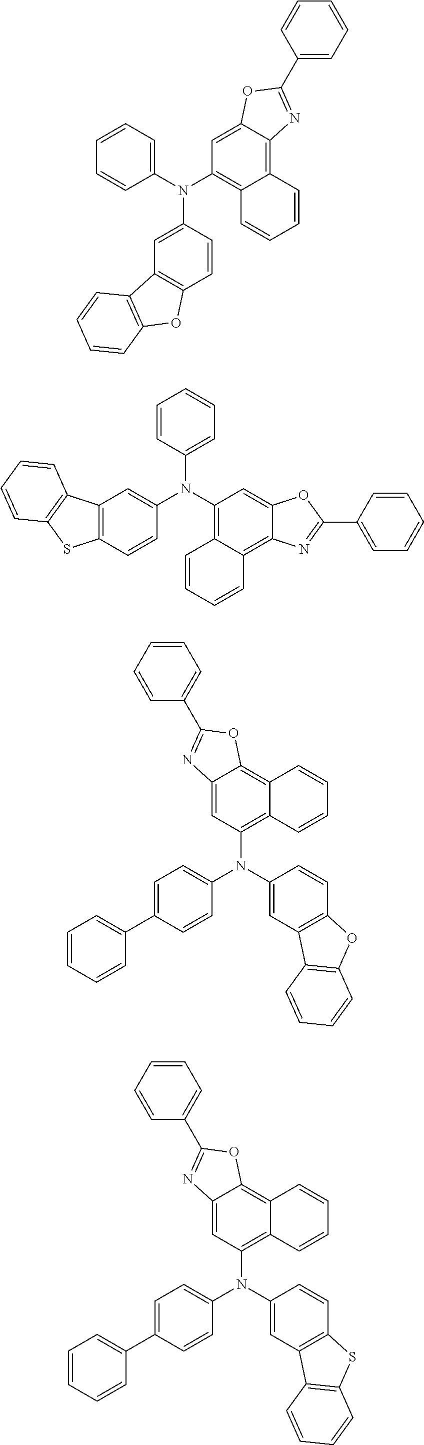 [SMILES: c1ccc(-c2nc3c(cc(N(c4ccccc4)c4ccc5oc6ccccc6c5c4)c4ccccc43)o2)cc1 ; c1ccc(-c2nc3c(cc(N(c4ccccc4)c4ccc5sc6ccccc6c5c4)c4ccccc43)o2)cc1 ; c1ccc(-c2ccc(N(c3ccc4oc5ccccc5c4c3)c3cc4nc(-c5ccccc5)oc4c4ccccc34)cc2)cc1 ; c1ccc(-c2ccc(N(c3ccc4sc5ccccc5c4c3)c3cc4nc(-c5ccccc5)oc4c4ccccc34)cc2)cc1]

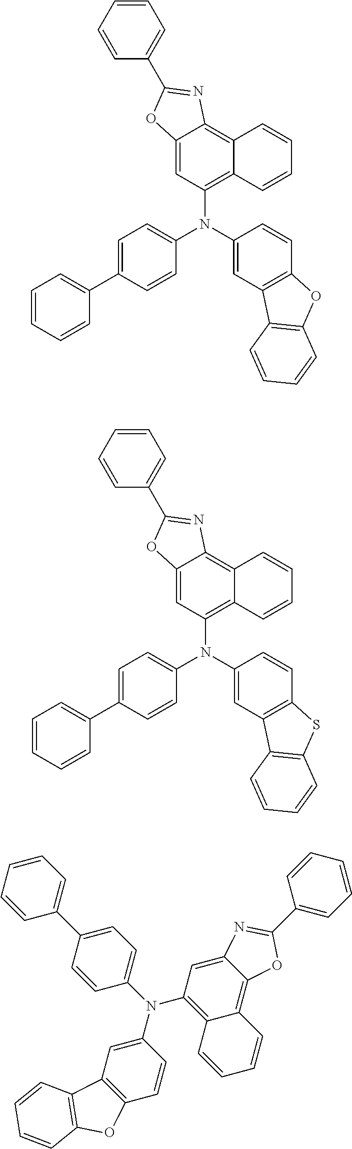

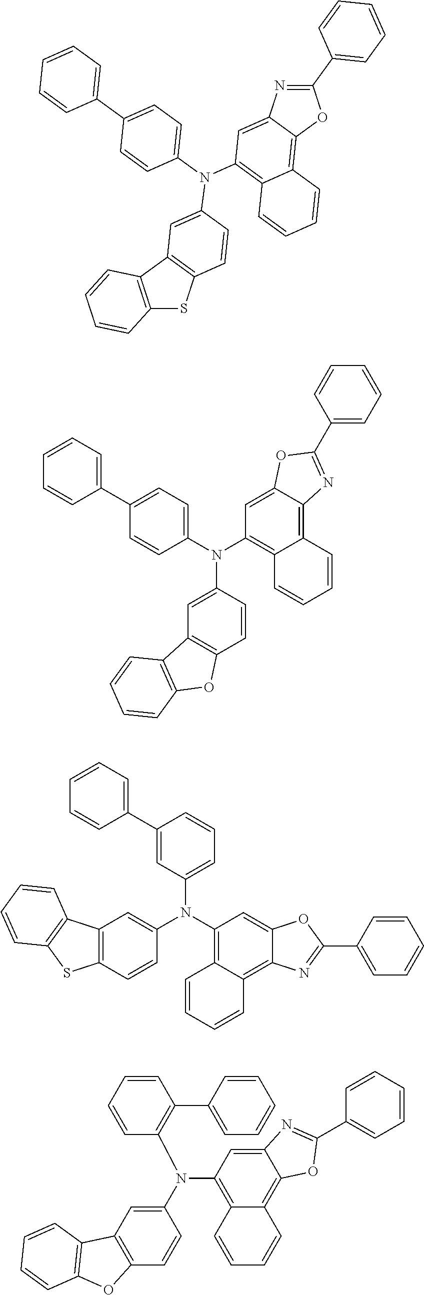 [SMILES: c1ccc(-c2ccc(N(c3ccc4sc5ccccc5c4c3)c3cc4nc(-c5ccccc5)oc4c4ccccc34)cc2)cc1 ; c1ccc(-c2ccc(N(c3ccc4oc5ccccc5c4c3)c3cc4oc(-c5ccccc5)nc4c4ccccc34)cc2)cc1 ; c1ccc(-c2cccc(N(c3ccc4sc5ccccc5c4c3)c3cc4oc(-c5ccccc5)nc4c4ccccc34)c2)cc1 ; c1ccc(-c2nc3cc(N(c4ccc5oc6ccccc6c5c4)c4ccccc4-c4ccccc4)c4ccccc4c3o2)cc1]

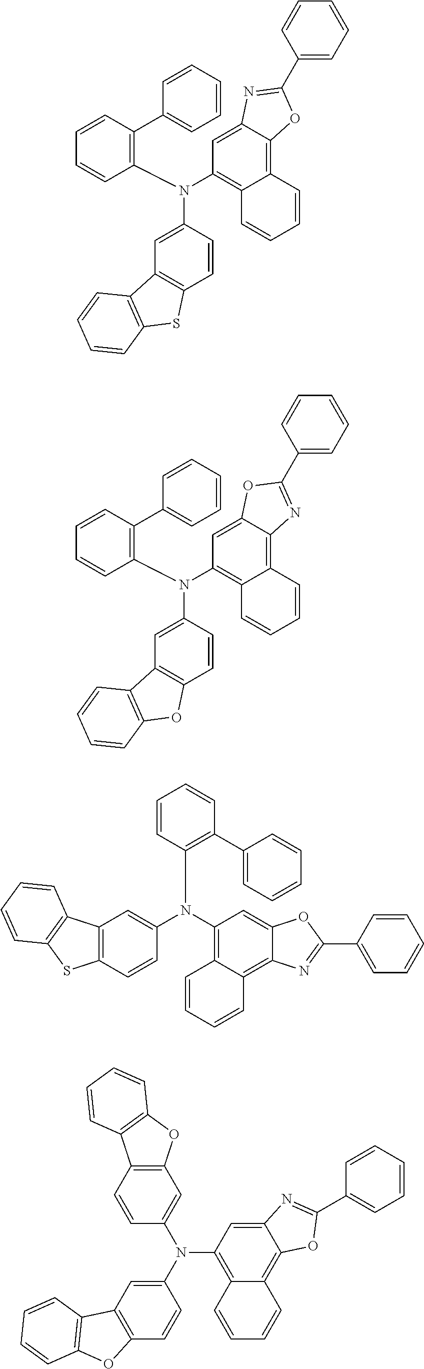 [SMILES: c1ccc(-c2nc3cc(N(c4ccc5sc6ccccc6c5c4)c4ccccc4-c4ccccc4)c4ccccc4c3o2)cc1 ; c1ccc(-c2nc3c(cc(N(c4ccc5oc6ccccc6c5c4)c4ccccc4-c4ccccc4)c4ccccc43)o2)cc1 ; c1ccc(-c2nc3c(cc(N(c4ccc5sc6ccccc6c5c4)c4ccccc4-c4ccccc4)c4ccccc43)o2)cc1 ; c1ccc(-c2nc3cc(N(c4ccc5c(c4)oc4ccccc45)c4ccc5oc6ccccc6c5c4)c4ccccc4c3o2)cc1]

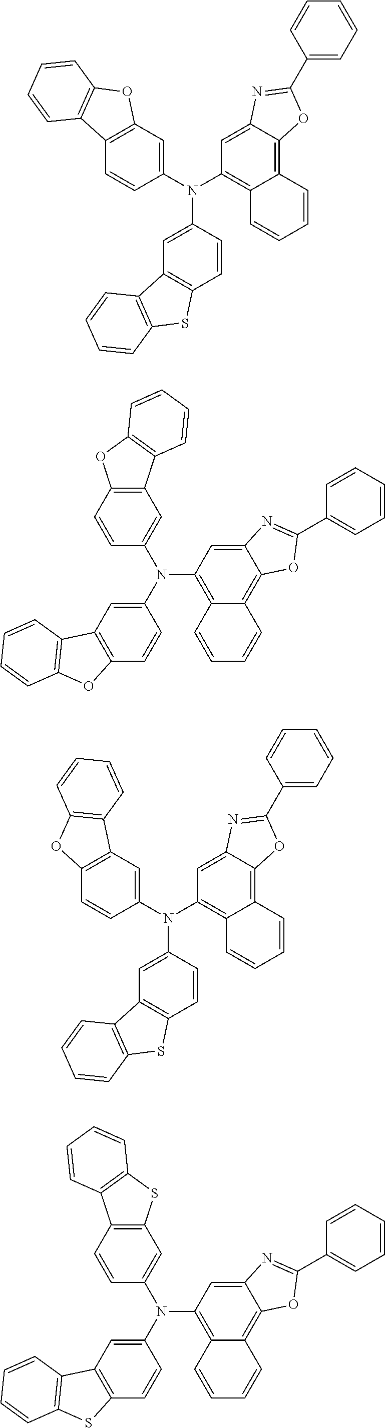

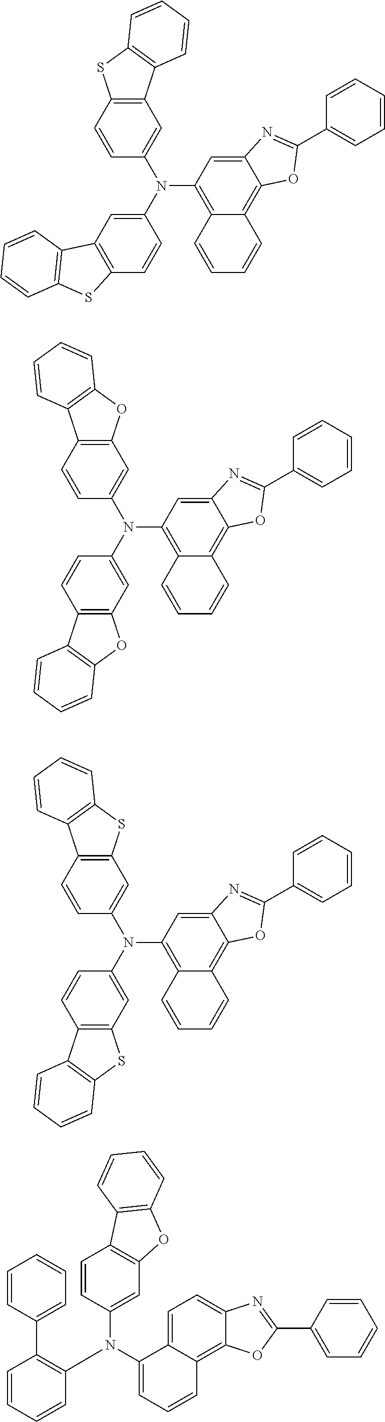

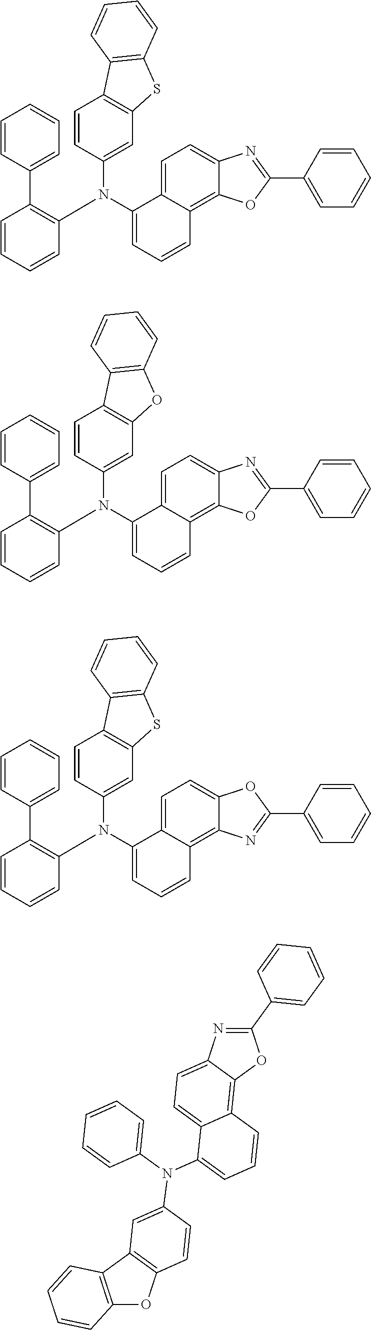 [SMILES: c1ccc(-c2nc3ccc4c(N(c5ccc6c(c5)sc5ccccc56)c5ccccc5-c5ccccc5)cccc4c3o2)cc1 ; c1ccc(-c2nc3ccc4c(N(c5ccc6c(c5)oc5ccccc56)c5ccccc5-c5ccccc5)cccc4c3o2)cc1 ; c1ccc(-c2nc3c(ccc4c(N(c5ccc6c(c5)sc5ccccc56)c5ccccc5-c5ccccc5)cccc43)o2)cc1 ; c1ccc(-c2nc3ccc4c(N(c5ccccc5)c5ccc6oc7ccccc7c6c5)cccc4c3o2)cc1]

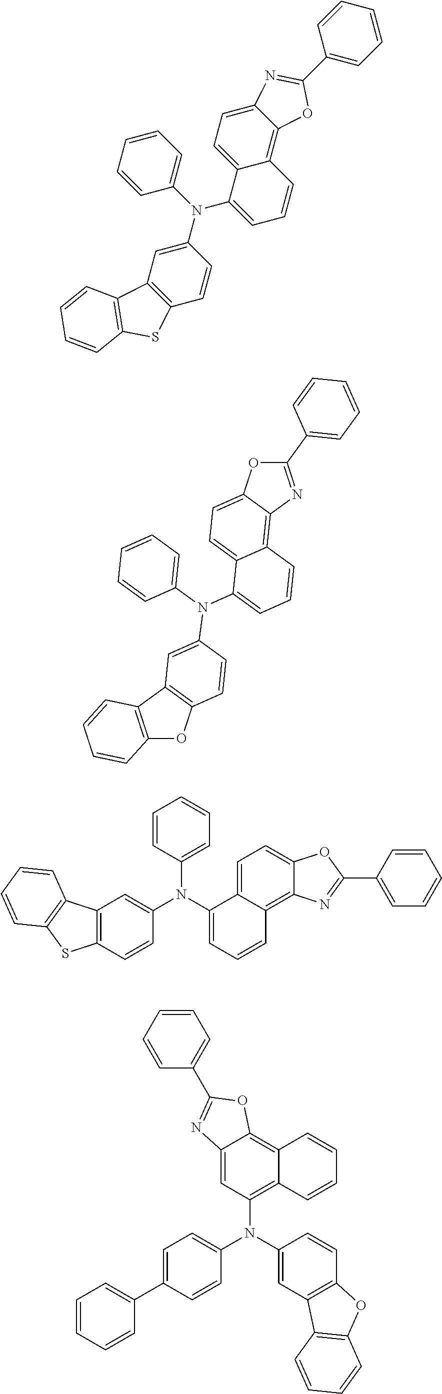 [SMILES: c1ccc(-c2nc3ccc4c(N(c5ccccc5)c5ccc6sc7ccccc7c6c5)cccc4c3o2)cc1 ; c1ccc(-c2nc3c(ccc4c(N(c5ccccc5)c5ccc6oc7ccccc7c6c5)cccc43)o2)cc1 ; c1ccc(-c2nc3c(ccc4c(N(c5ccccc5)c5ccc6sc7ccccc7c6c5)cccc43)o2)cc1 ; c1ccc(-c2ccc(N(c3ccc4oc5ccccc5c4c3)c3cc4nc(-c5ccccc5)oc4c4ccccc34)cc2)cc1]

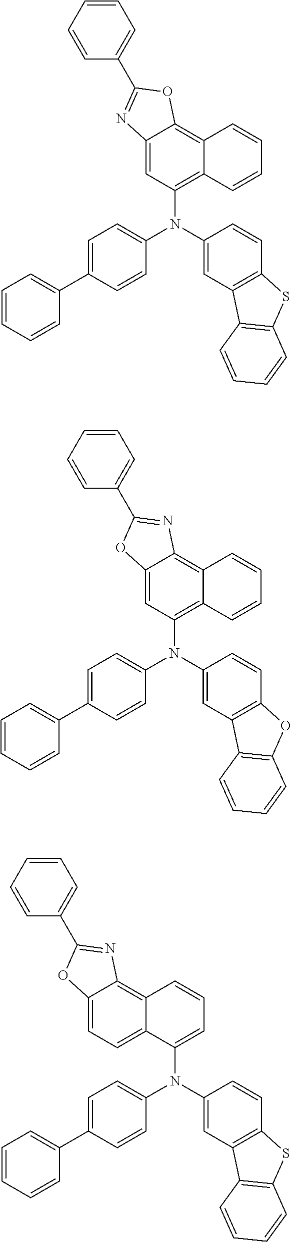 [SMILES: c1ccc(-c2ccc(N(c3ccc4sc5ccccc5c4c3)c3cc4nc(-c5ccccc5)oc4c4ccccc34)cc2)cc1 ; c1ccc(-c2ccc(N(c3ccc4oc5ccccc5c4c3)c3cc4oc(-c5ccccc5)nc4c4ccccc34)cc2)cc1 ; c1ccc(-c2ccc(N(c3ccc4sc5ccccc5c4c3)c3cccc4c3ccc3oc(-c5ccccc5)nc34)cc2)cc1]

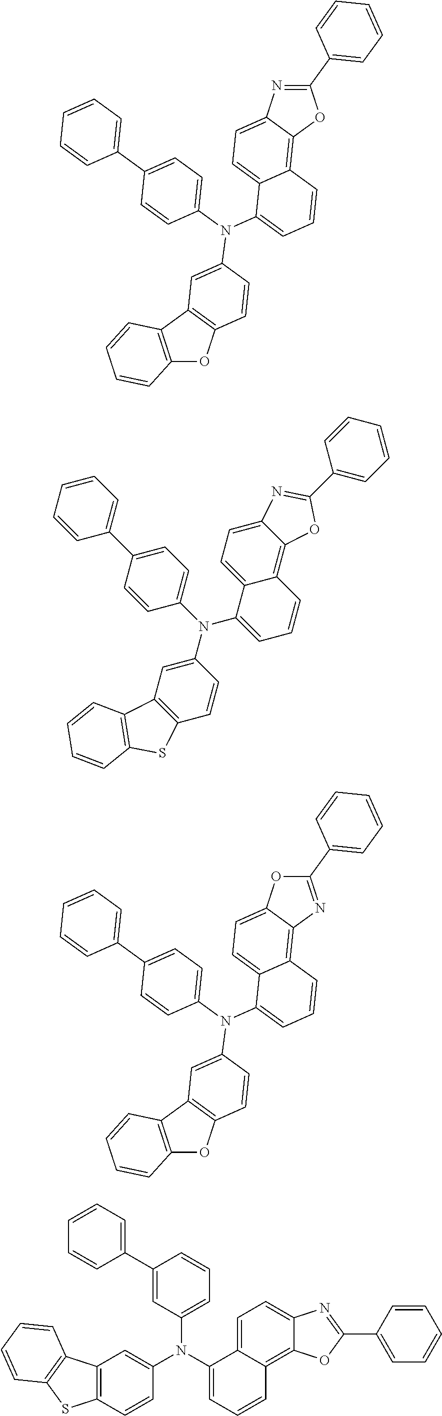

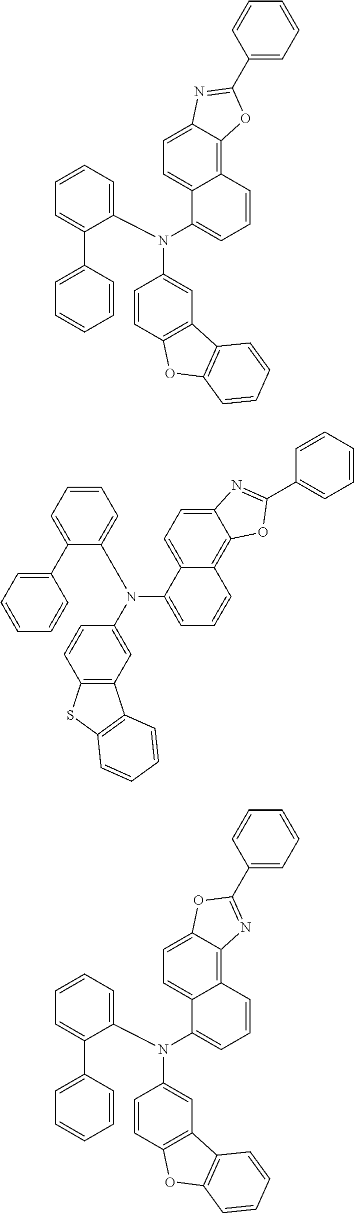 [SMILES: c1ccc(-c2nc3ccc4c(N(c5ccc6oc7ccccc7c6c5)c5ccccc5-c5ccccc5)cccc4c3o2)cc1 ; c1ccc(-c2nc3ccc4c(N(c5ccc6sc7ccccc7c6c5)c5ccccc5-c5ccccc5)cccc4c3o2)cc1 ; c1ccc(-c2nc3c(ccc4c(N(c5ccc6oc7ccccc7c6c5)c5ccccc5-c5ccccc5)cccc43)o2)cc1]

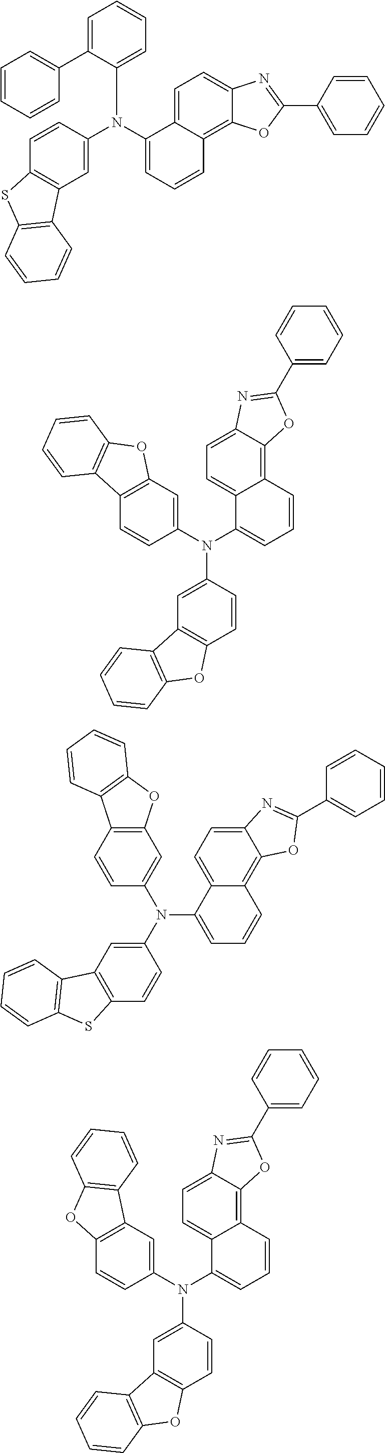

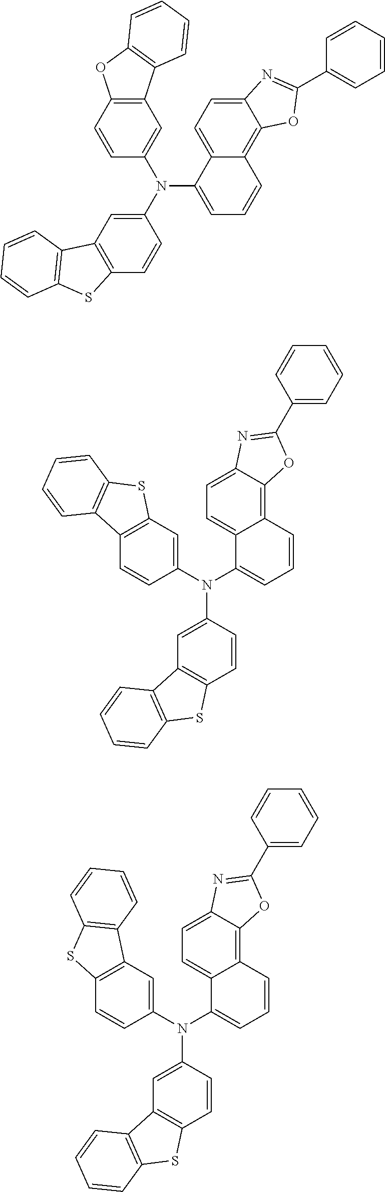 [SMILES: c1ccc(-c2nc3ccc4c(N(c5ccc6oc7ccccc7c6c5)c5ccc6sc7ccccc7c6c5)cccc4c3o2)cc1 ; c1ccc(-c2nc3ccc4c(N(c5ccc6c(c5)sc5ccccc56)c5ccc6sc7ccccc7c6c5)cccc4c3o2)cc1 ; c1ccc(-c2nc3ccc4c(N(c5ccc6sc7ccccc7c6c5)c5ccc6sc7ccccc7c6c5)cccc4c3o2)cc1]

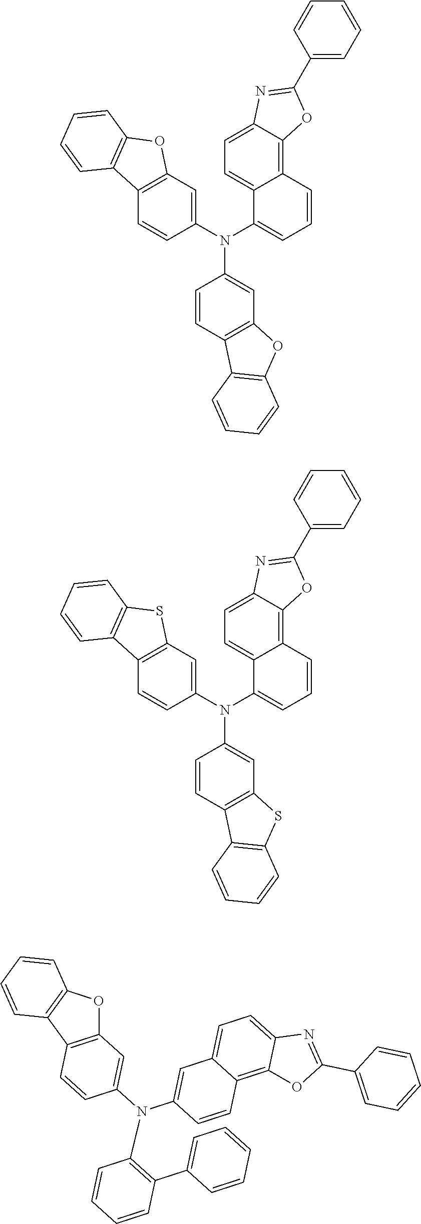 [SMILES: c1ccc(-c2nc3ccc4c(N(c5ccc6c(c5)oc5ccccc56)c5ccc6c(c5)oc5ccccc56)cccc4c3o2)cc1 ; c1ccc(-c2nc3ccc4c(N(c5ccc6c(c5)sc5ccccc56)c5ccc6c(c5)sc5ccccc56)cccc4c3o2)cc1 ; c1ccc(-c2nc3ccc4cc(N(c5ccc6c(c5)oc5ccccc56)c5ccccc5-c5ccccc5)ccc4c3o2)cc1]

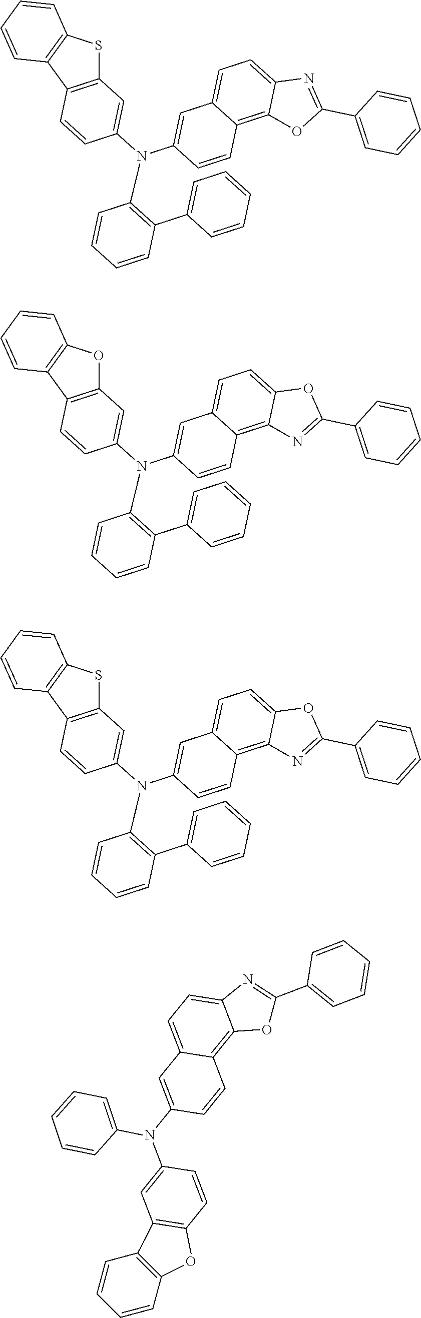 [SMILES: c1ccc(-c2nc3ccc4cc(N(c5ccc6c(c5)sc5ccccc56)c5ccccc5-c5ccccc5)ccc4c3o2)cc1 ; c1ccc(-c2nc3c(ccc4cc(N(c5ccc6c(c5)oc5ccccc56)c5ccccc5-c5ccccc5)ccc43)o2)cc1 ; c1ccc(-c2nc3c(ccc4cc(N(c5ccc6c(c5)sc5ccccc56)c5ccccc5-c5ccccc5)ccc43)o2)cc1 ; c1ccc(-c2nc3ccc4cc(N(c5ccccc5)c5ccc6oc7ccccc7c6c5)ccc4c3o2)cc1]

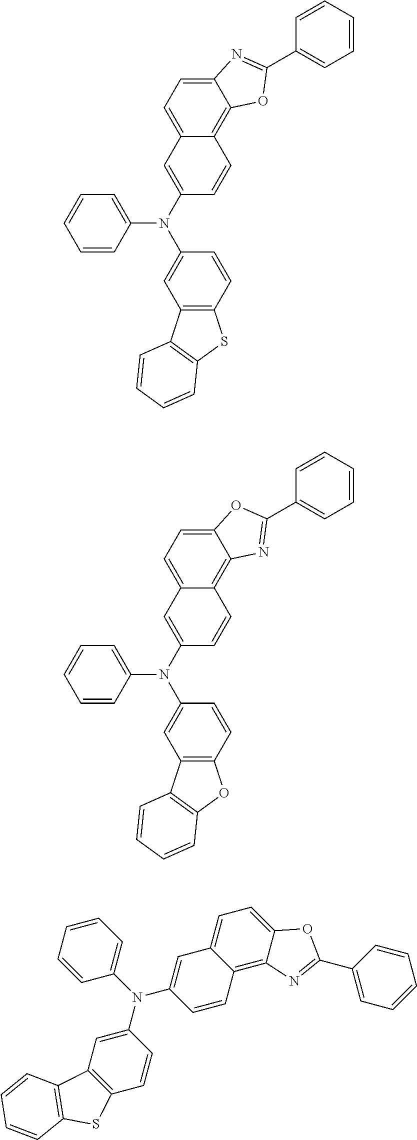 [SMILES: c1ccc(-c2nc3ccc4cc(N(c5ccccc5)c5ccc6sc7ccccc7c6c5)ccc4c3o2)cc1 ; c1ccc(-c2nc3c(ccc4cc(N(c5ccccc5)c5ccc6oc7ccccc7c6c5)ccc43)o2)cc1 ; c1ccc(-c2nc3c(ccc4cc(N(c5ccccc5)c5ccc6sc7ccccc7c6c5)ccc43)o2)cc1]

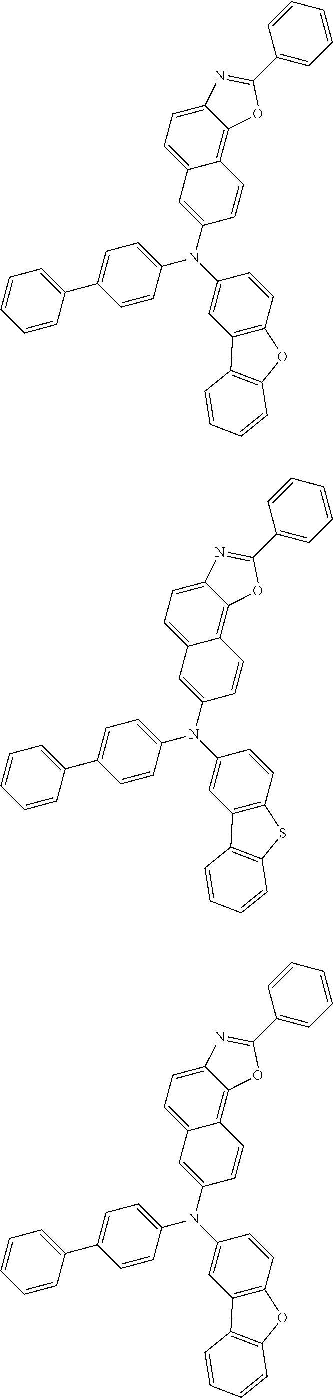 [SMILES: c1ccc(-c2ccc(N(c3ccc4c(ccc5nc(-c6ccccc6)oc54)c3)c3ccc4oc5ccccc5c4c3)cc2)cc1 ; c1ccc(-c2ccc(N(c3ccc4c(ccc5nc(-c6ccccc6)oc54)c3)c3ccc4sc5ccccc5c4c3)cc2)cc1 ; c1ccc(-c2ccc(N(c3ccc4c(ccc5nc(-c6ccccc6)oc54)c3)c3ccc4oc5ccccc5c4c3)cc2)cc1]

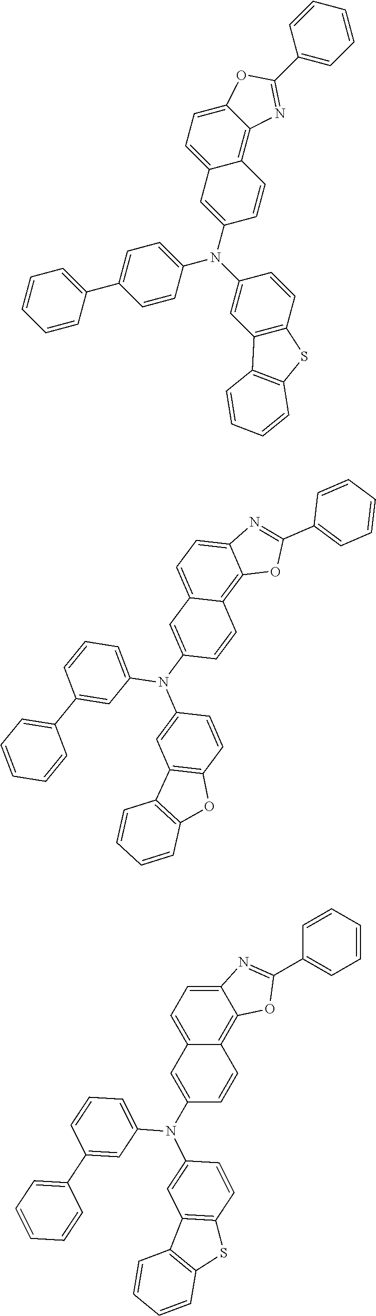 [SMILES: c1ccc(-c2ccc(N(c3ccc4c(ccc5oc(-c6ccccc6)nc54)c3)c3ccc4sc5ccccc5c4c3)cc2)cc1 ; c1ccc(-c2cccc(N(c3ccc4c(ccc5nc(-c6ccccc6)oc54)c3)c3ccc4oc5ccccc5c4c3)c2)cc1 ; c1ccc(-c2cccc(N(c3ccc4c(ccc5nc(-c6ccccc6)oc54)c3)c3ccc4sc5ccccc5c4c3)c2)cc1]

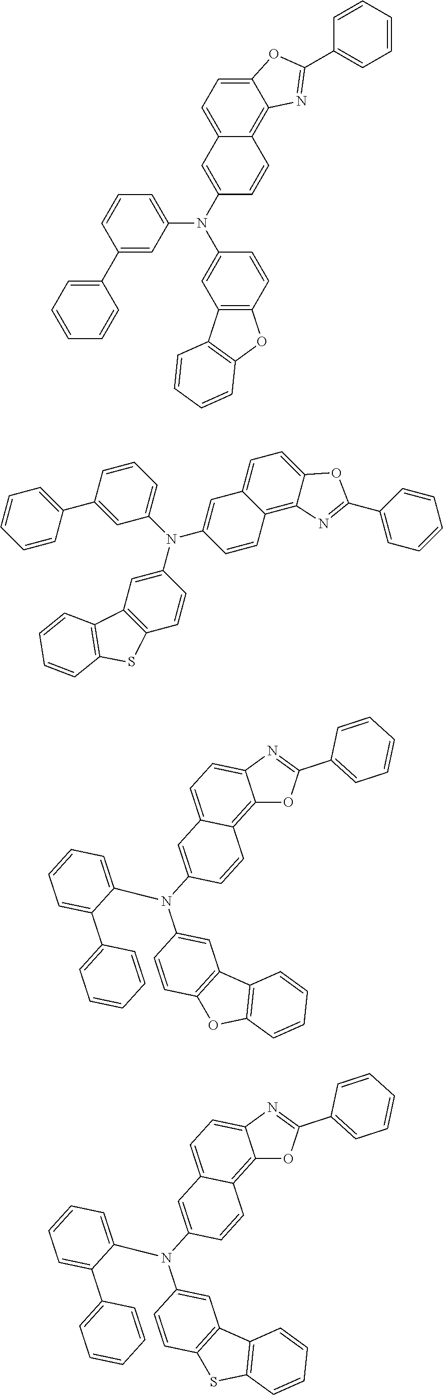 [SMILES: c1ccc(-c2cccc(N(c3ccc4c(ccc5oc(-c6ccccc6)nc54)c3)c3ccc4oc5ccccc5c4c3)c2)cc1 ; c1ccc(-c2cccc(N(c3ccc4c(ccc5oc(-c6ccccc6)nc54)c3)c3ccc4sc5ccccc5c4c3)c2)cc1 ; c1ccc(-c2nc3ccc4cc(N(c5ccc6oc7ccccc7c6c5)c5ccccc5-c5ccccc5)ccc4c3o2)cc1 ; c1ccc(-c2nc3ccc4cc(N(c5ccc6sc7ccccc7c6c5)c5ccccc5-c5ccccc5)ccc4c3o2)cc1]

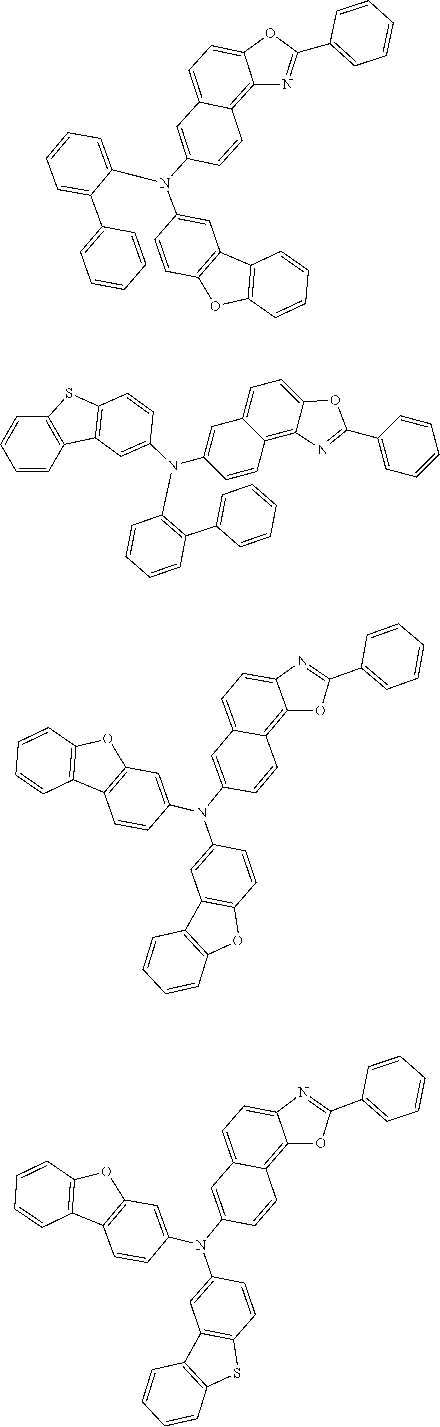 [SMILES: c1ccc(-c2nc3c(ccc4cc(N(c5ccc6oc7ccccc7c6c5)c5ccccc5-c5ccccc5)ccc43)o2)cc1 ; c1ccc(-c2nc3c(ccc4cc(N(c5ccc6sc7ccccc7c6c5)c5ccccc5-c5ccccc5)ccc43)o2)cc1 ; c1ccc(-c2nc3ccc4cc(N(c5ccc6c(c5)oc5ccccc56)c5ccc6oc7ccccc7c6c5)ccc4c3o2)cc1 ; c1ccc(-c2nc3ccc4cc(N(c5ccc6c(c5)oc5ccccc56)c5ccc6sc7ccccc7c6c5)ccc4c3o2)cc1]

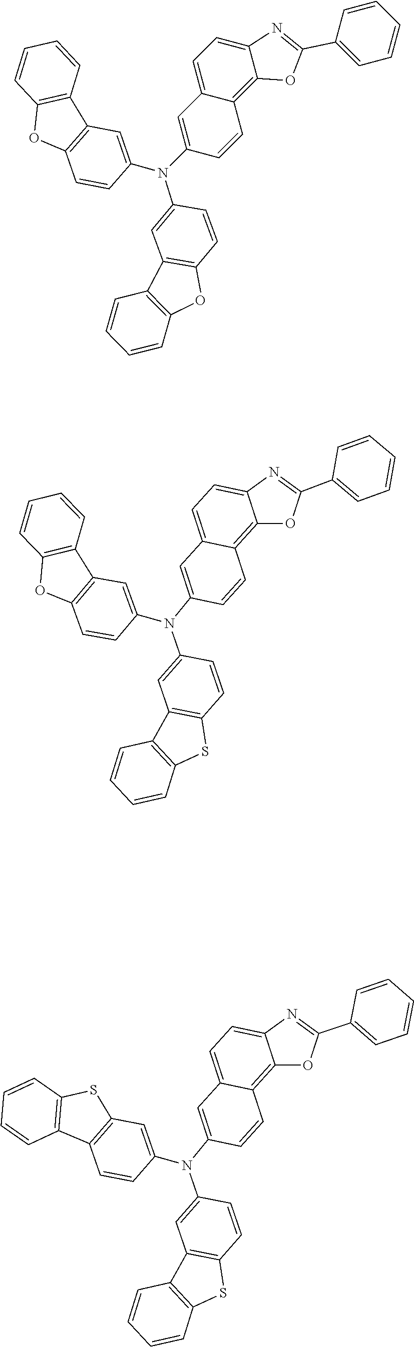 [SMILES: c1ccc(-c2nc3ccc4cc(N(c5ccc6oc7ccccc7c6c5)c5ccc6oc7ccccc7c6c5)ccc4c3o2)cc1 ; c1ccc(-c2nc3ccc4cc(N(c5ccc6oc7ccccc7c6c5)c5ccc6sc7ccccc7c6c5)ccc4c3o2)cc1 ; c1ccc(-c2nc3ccc4cc(N(c5ccc6c(c5)sc5ccccc56)c5ccc6sc7ccccc7c6c5)ccc4c3o2)cc1]

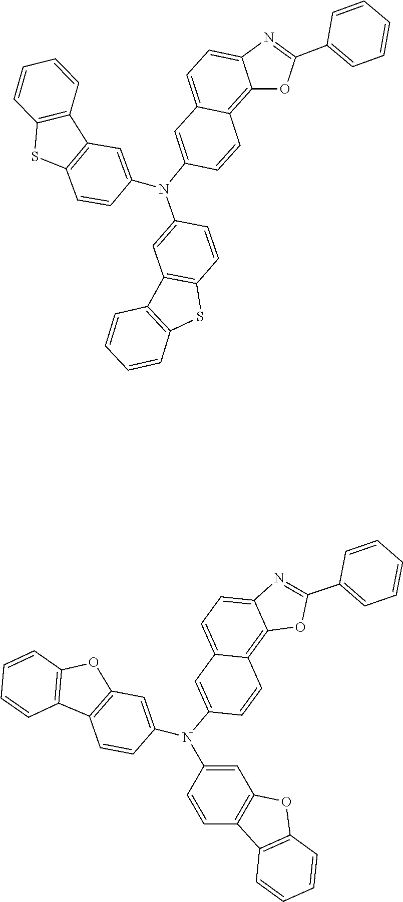 [SMILES: c1ccc(-c2nc3ccc4cc(N(c5ccc6sc7ccccc7c6c5)c5ccc6sc7ccccc7c6c5)ccc4c3o2)cc1 ; c1ccc(-c2nc3ccc4cc(N(c5ccc6c(c5)oc5ccccc56)c5ccc6c(c5)oc5ccccc56)ccc4c3o2)cc1]

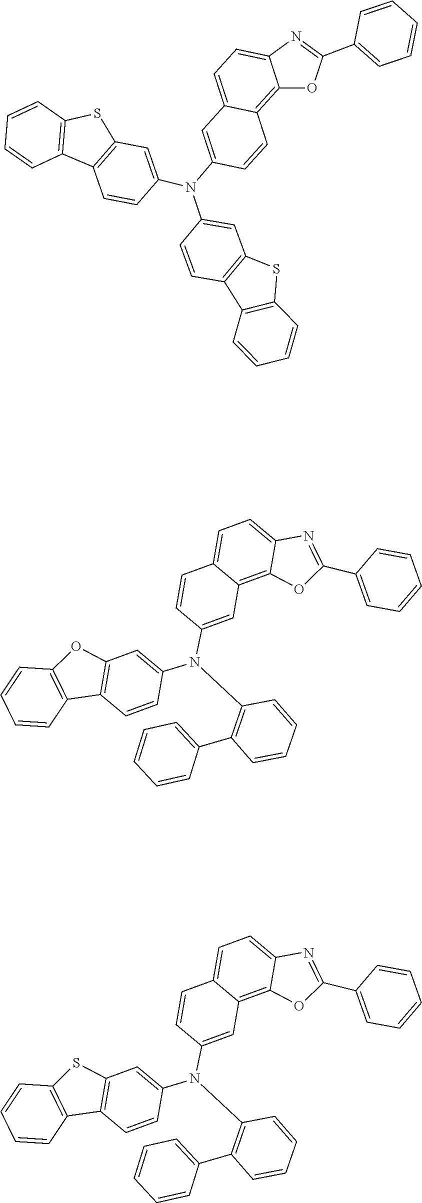 [SMILES: c1ccc(-c2nc3ccc4cc(N(c5ccc6c(c5)sc5ccccc56)c5ccc6c(c5)sc5ccccc56)ccc4c3o2)cc1 ; c1ccc(-c2nc3ccc4ccc(N(c5ccc6c(c5)oc5ccccc56)c5ccccc5-c5ccccc5)cc4c3o2)cc1 ; c1ccc(-c2nc3ccc4ccc(N(c5ccc6c(c5)sc5ccccc56)c5ccccc5-c5ccccc5)cc4c3o2)cc1]

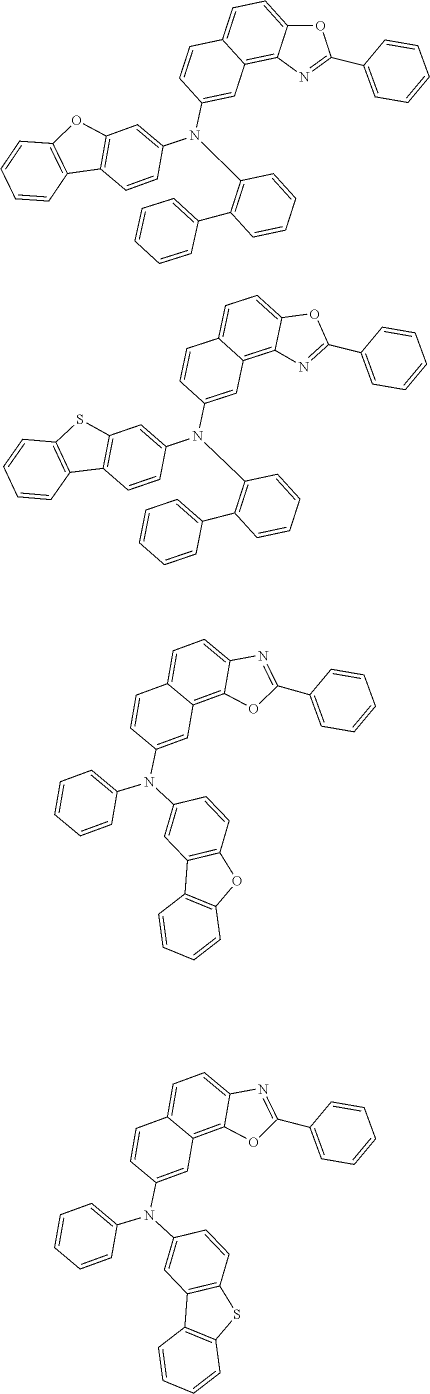 [SMILES: c1ccc(-c2nc3c(ccc4ccc(N(c5ccc6c(c5)oc5ccccc56)c5ccccc5-c5ccccc5)cc43)o2)cc1 ; c1ccc(-c2nc3c(ccc4ccc(N(c5ccc6c(c5)sc5ccccc56)c5ccccc5-c5ccccc5)cc43)o2)cc1 ; c1ccc(-c2nc3ccc4ccc(N(c5ccccc5)c5ccc6oc7ccccc7c6c5)cc4c3o2)cc1 ; c1ccc(-c2nc3ccc4ccc(N(c5ccccc5)c5ccc6sc7ccccc7c6c5)cc4c3o2)cc1]

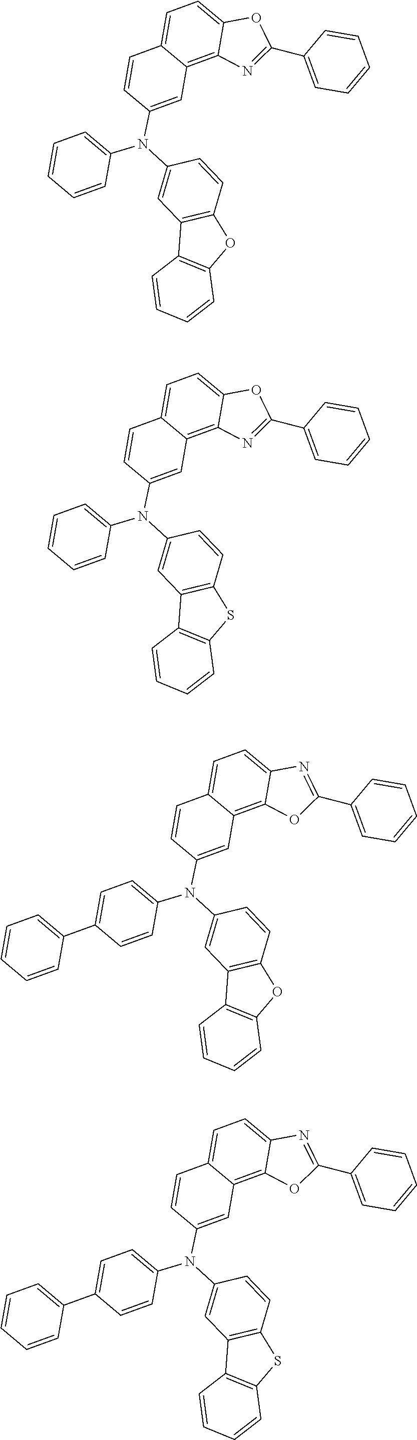 [SMILES: c1ccc(-c2nc3c(ccc4ccc(N(c5ccccc5)c5ccc6oc7ccccc7c6c5)cc43)o2)cc1 ; c1ccc(-c2nc3c(ccc4ccc(N(c5ccccc5)c5ccc6sc7ccccc7c6c5)cc43)o2)cc1 ; c1ccc(-c2ccc(N(c3ccc4oc5ccccc5c4c3)c3ccc4ccc5nc(-c6ccccc6)oc5c4c3)cc2)cc1 ; c1ccc(-c2ccc(N(c3ccc4sc5ccccc5c4c3)c3ccc4ccc5nc(-c6ccccc6)oc5c4c3)cc2)cc1]

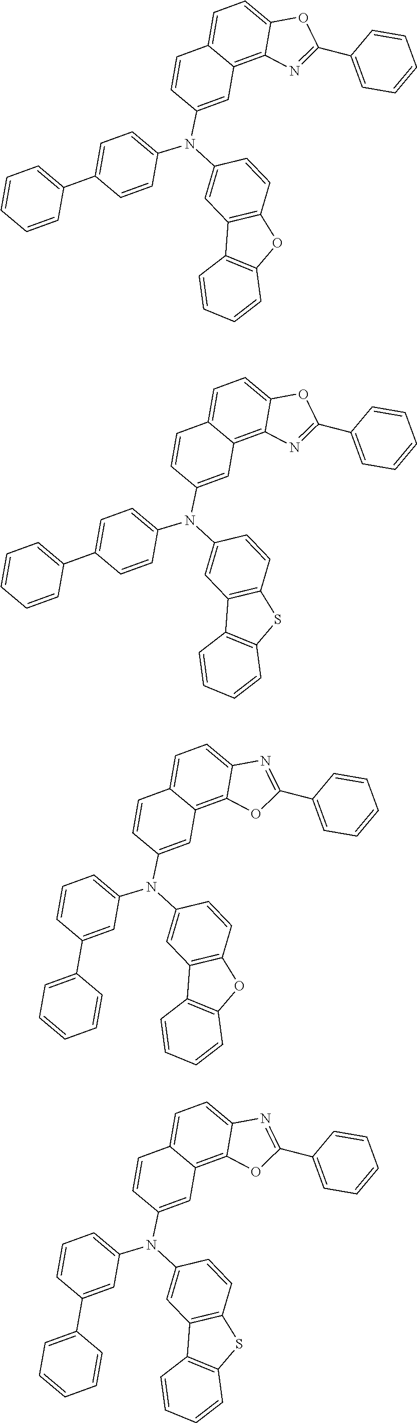

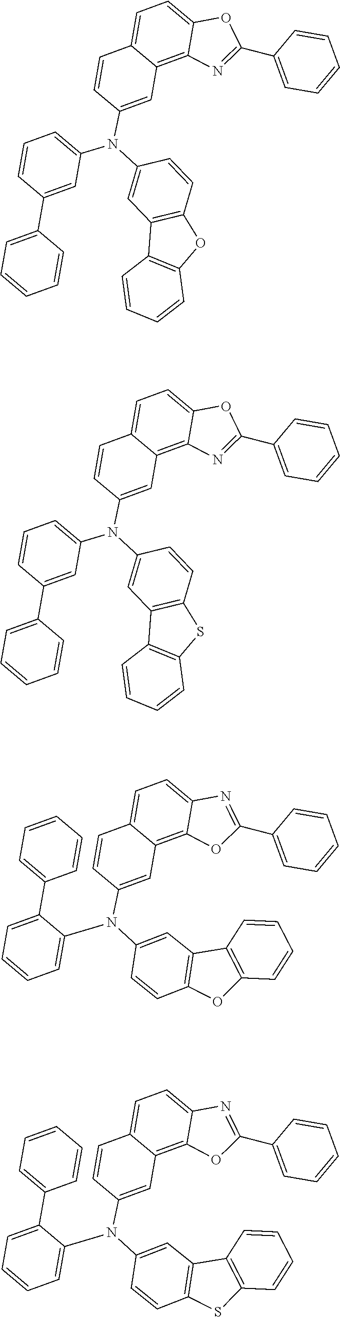 [SMILES: c1ccc(-c2cccc(N(c3ccc4oc5ccccc5c4c3)c3ccc4ccc5oc(-c6ccccc6)nc5c4c3)c2)cc1 ; c1ccc(-c2cccc(N(c3ccc4sc5ccccc5c4c3)c3ccc4ccc5oc(-c6ccccc6)nc5c4c3)c2)cc1 ; c1ccc(-c2nc3ccc4ccc(N(c5ccc6oc7ccccc7c6c5)c5ccccc5-c5ccccc5)cc4c3o2)cc1 ; c1ccc(-c2nc3ccc4ccc(N(c5ccc6sc7ccccc7c6c5)c5ccccc5-c5ccccc5)cc4c3o2)cc1]

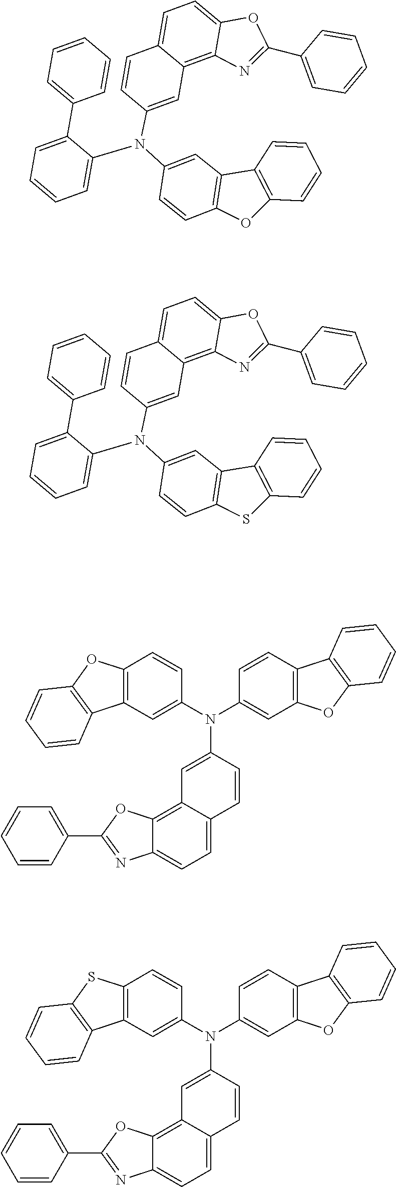 [SMILES: c1ccc(-c2nc3c(ccc4ccc(N(c5ccc6oc7ccccc7c6c5)c5ccccc5-c5ccccc5)cc43)o2)cc1 ; c1ccc(-c2nc3c(ccc4ccc(N(c5ccc6sc7ccccc7c6c5)c5ccccc5-c5ccccc5)cc43)o2)cc1 ; c1ccc(-c2nc3ccc4ccc(N(c5ccc6c(c5)oc5ccccc56)c5ccc6oc7ccccc7c6c5)cc4c3o2)cc1 ; c1ccc(-c2nc3ccc4ccc(N(c5ccc6c(c5)oc5ccccc56)c5ccc6sc7ccccc7c6c5)cc4c3o2)cc1]

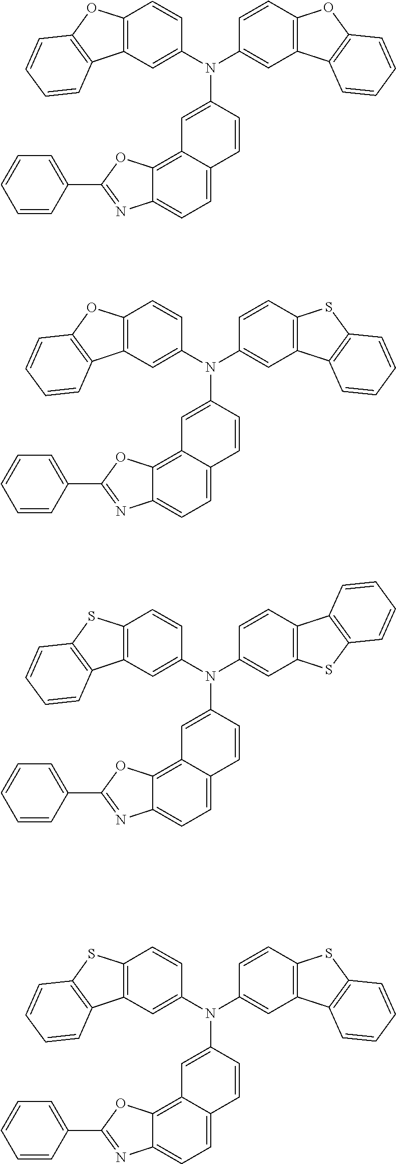 [SMILES: c1ccc(-c2nc3ccc4ccc(N(c5ccc6oc7ccccc7c6c5)c5ccc6oc7ccccc7c6c5)cc4c3o2)cc1 ; c1ccc(-c2nc3ccc4ccc(N(c5ccc6oc7ccccc7c6c5)c5ccc6sc7ccccc7c6c5)cc4c3o2)cc1 ; c1ccc(-c2nc3ccc4ccc(N(c5ccc6c(c5)sc5ccccc56)c5ccc6sc7ccccc7c6c5)cc4c3o2)cc1 ; c1ccc(-c2nc3ccc4ccc(N(c5ccc6sc7ccccc7c6c5)c5ccc6sc7ccccc7c6c5)cc4c3o2)cc1]

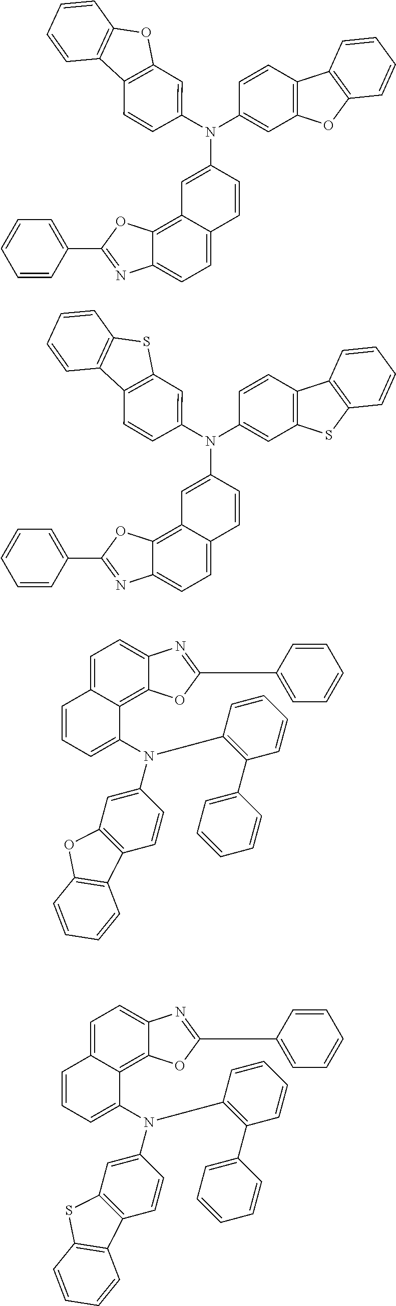

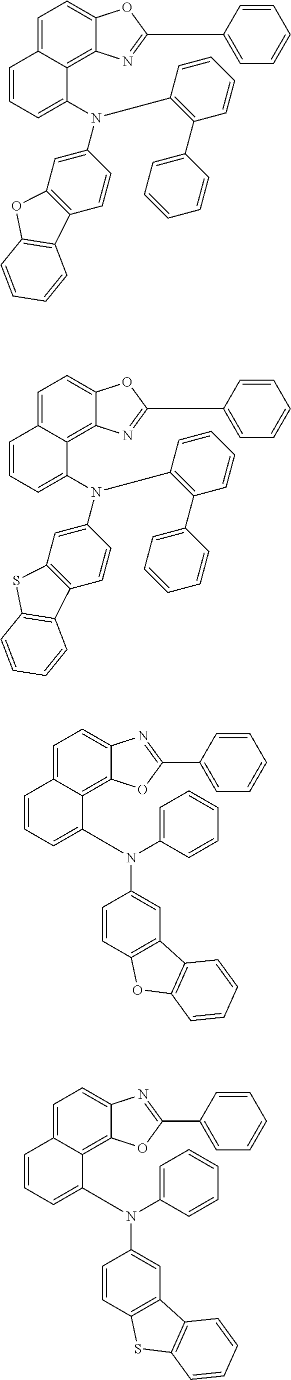 [SMILES: c1ccc(-c2nc3c(ccc4cccc(N(c5ccc6c(c5)oc5ccccc56)c5ccccc5-c5ccccc5)c43)o2)cc1 ; c1ccc(-c2nc3c(ccc4cccc(N(c5ccc6c(c5)sc5ccccc56)c5ccccc5-c5ccccc5)c43)o2)cc1 ; c1ccc(-c2nc3ccc4cccc(N(c5ccccc5)c5ccc6oc7ccccc7c6c5)c4c3o2)cc1 ; c1ccc(-c2nc3ccc4cccc(N(c5ccccc5)c5ccc6sc7ccccc7c6c5)c4c3o2)cc1]

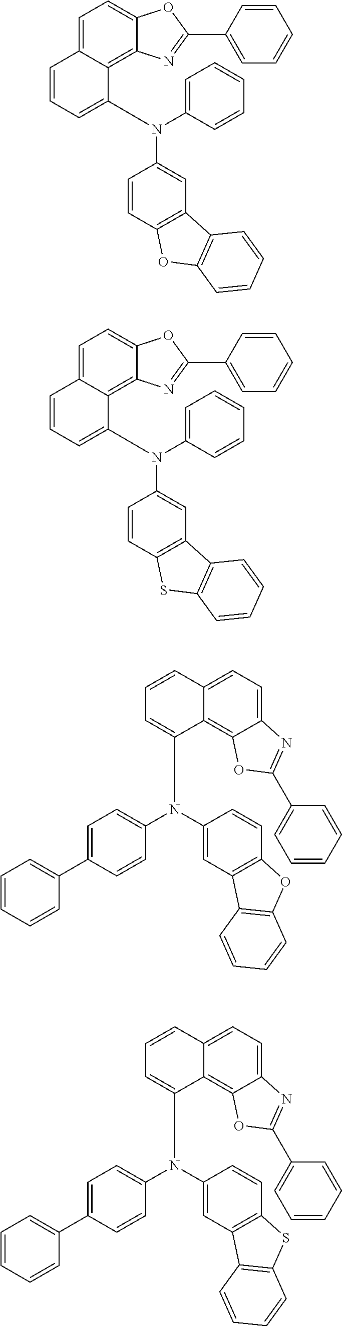

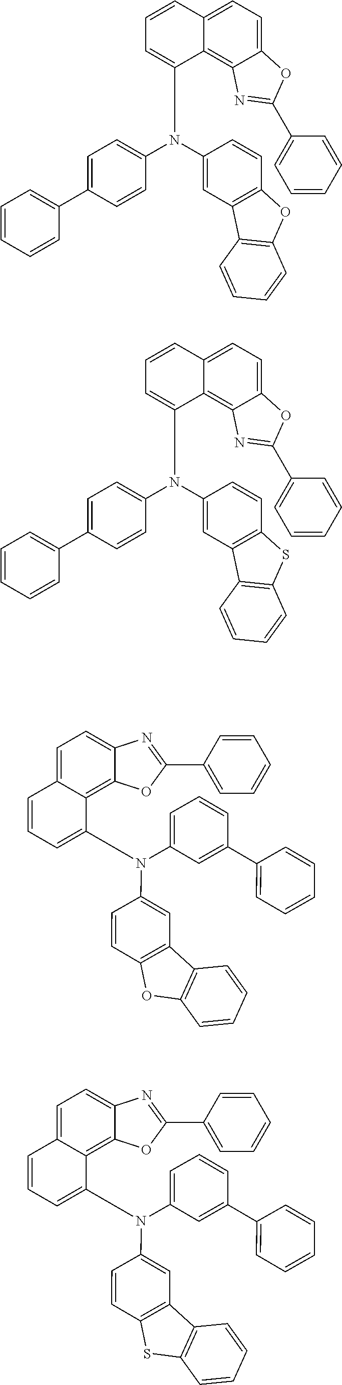 [SMILES: c1ccc(-c2ccc(N(c3ccc4oc5ccccc5c4c3)c3cccc4ccc5oc(-c6ccccc6)nc5c34)cc2)cc1 ; c1ccc(-c2ccc(N(c3ccc4sc5ccccc5c4c3)c3cccc4ccc5oc(-c6ccccc6)nc5c34)cc2)cc1 ; c1ccc(-c2cccc(N(c3ccc4oc5ccccc5c4c3)c3cccc4ccc5nc(-c6ccccc6)oc5c34)c2)cc1 ; c1ccc(-c2cccc(N(c3ccc4sc5ccccc5c4c3)c3cccc4ccc5nc(-c6ccccc6)oc5c34)c2)cc1]

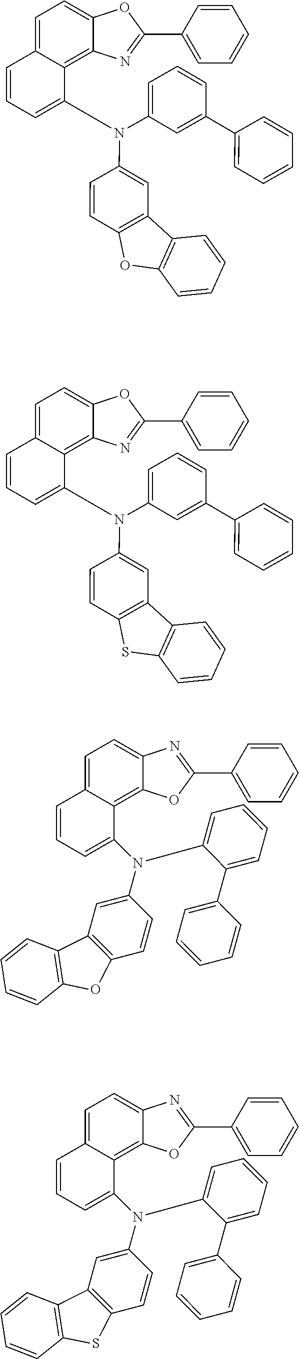 [SMILES: c1ccc(-c2cccc(N(c3ccc4oc5ccccc5c4c3)c3cccc4ccc5oc(-c6ccccc6)nc5c34)c2)cc1 ; c1ccc(-c2cccc(N(c3ccc4sc5ccccc5c4c3)c3cccc4ccc5oc(-c6ccccc6)nc5c34)c2)cc1 ; c1ccc(-c2nc3ccc4cccc(N(c5ccc6oc7ccccc7c6c5)c5ccccc5-c5ccccc5)c4c3o2)cc1 ; c1ccc(-c2nc3ccc4cccc(N(c5ccc6sc7ccccc7c6c5)c5ccccc5-c5ccccc5)c4c3o2)cc1]

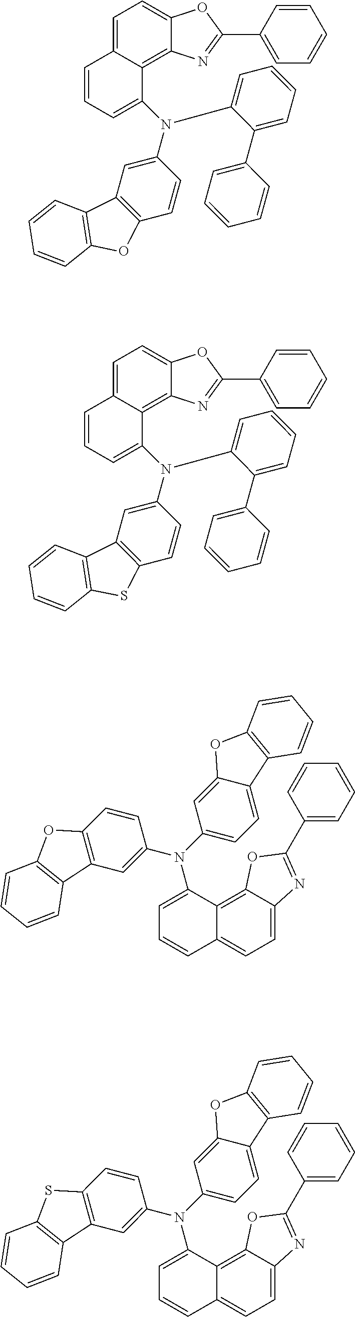 [SMILES: c1ccc(-c2nc3c(ccc4cccc(N(c5ccc6oc7ccccc7c6c5)c5ccccc5-c5ccccc5)c43)o2)cc1 ; c1ccc(-c2nc3c(ccc4cccc(N(c5ccc6sc7ccccc7c6c5)c5ccccc5-c5ccccc5)c43)o2)cc1 ; c1ccc(-c2nc3ccc4cccc(N(c5ccc6c(c5)oc5ccccc56)c5ccc6oc7ccccc7c6c5)c4c3o2)cc1 ; c1ccc(-c2nc3ccc4cccc(N(c5ccc6c(c5)oc5ccccc56)c5ccc6sc7ccccc7c6c5)c4c3o2)cc1]

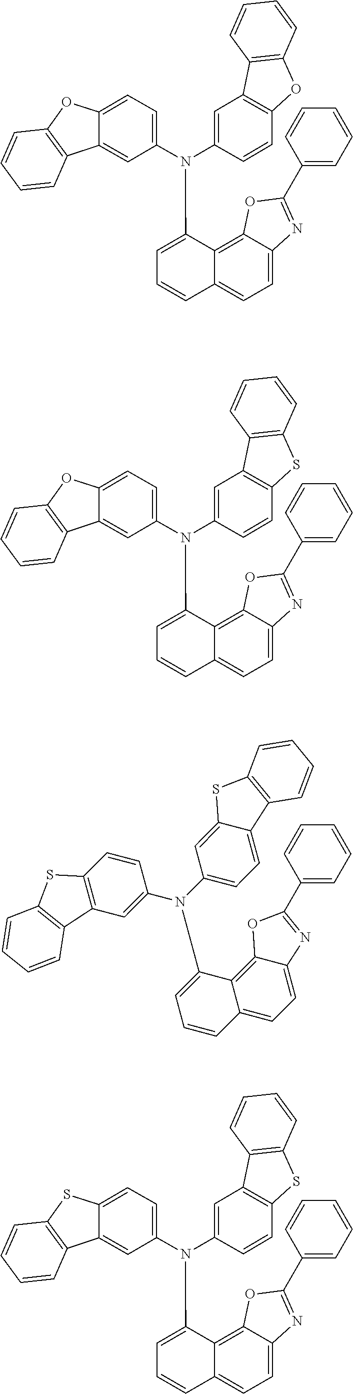 [SMILES: c1ccc(-c2nc3ccc4cccc(N(c5ccc6oc7ccccc7c6c5)c5ccc6oc7ccccc7c6c5)c4c3o2)cc1 ; c1ccc(-c2nc3ccc4cccc(N(c5ccc6oc7ccccc7c6c5)c5ccc6sc7ccccc7c6c5)c4c3o2)cc1 ; c1ccc(-c2nc3ccc4cccc(N(c5ccc6c(c5)sc5ccccc56)c5ccc6sc7ccccc7c6c5)c4c3o2)cc1 ; c1ccc(-c2nc3ccc4cccc(N(c5ccc6sc7ccccc7c6c5)c5ccc6sc7ccccc7c6c5)c4c3o2)cc1]

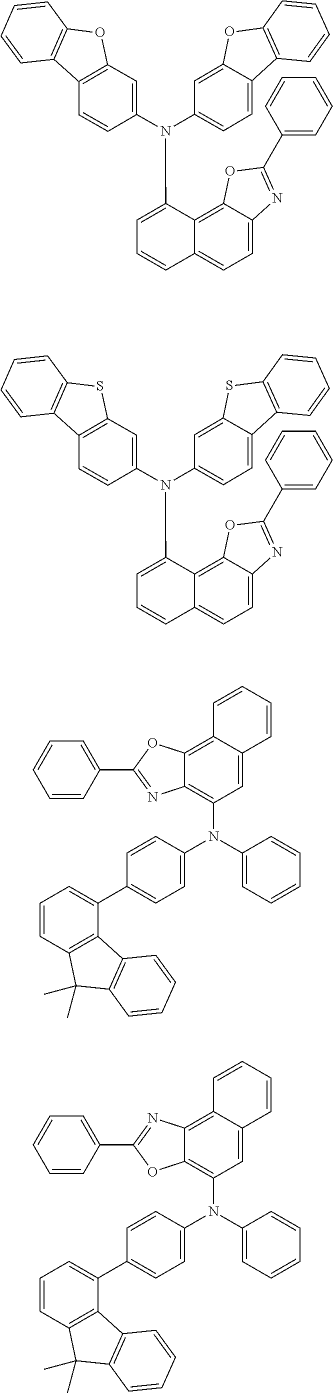 [SMILES: c1ccc(-c2nc3ccc4cccc(N(c5ccc6c(c5)oc5ccccc56)c5ccc6c(c5)oc5ccccc56)c4c3o2)cc1 ; c1ccc(-c2nc3ccc4cccc(N(c5ccc6c(c5)sc5ccccc56)c5ccc6c(c5)sc5ccccc56)c4c3o2)cc1 ; CC1(C)c2ccccc2-c2c(-c3ccc(N(c4ccccc4)c4cc5ccccc5c5oc(-c6ccccc6)nc45)cc3)cccc21 ; CC1(C)c2ccccc2-c2c(-c3ccc(N(c4ccccc4)c4cc5ccccc5c5nc(-c6ccccc6)oc45)cc3)cccc21]

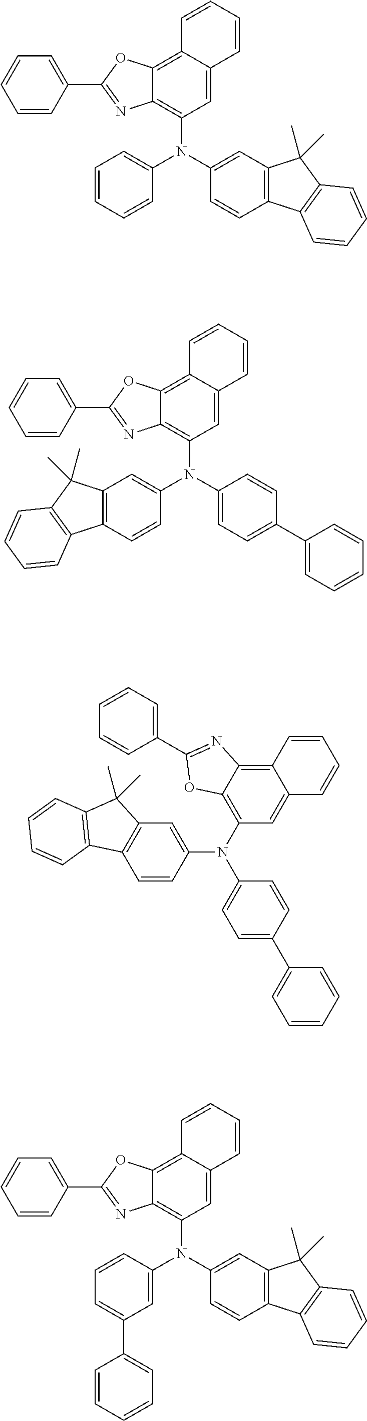 [SMILES: CC1(C)c2ccccc2-c2ccc(N(c3ccccc3)c3cc4ccccc4c4oc(-c5ccccc5)nc34)cc21 ; CC1(C)c2ccccc2-c2ccc(N(c3ccc(-c4ccccc4)cc3)c3cc4ccccc4c4oc(-c5ccccc5)nc34)cc21 ; CC1(C)c2ccccc2-c2ccc(N(c3ccc(-c4ccccc4)cc3)c3cc4ccccc4c4nc(-c5ccccc5)oc34)cc21 ; CC1(C)c2ccccc2-c2ccc(N(c3cccc(-c4ccccc4)c3)c3cc4ccccc4c4oc(-c5ccccc5)nc34)cc21]

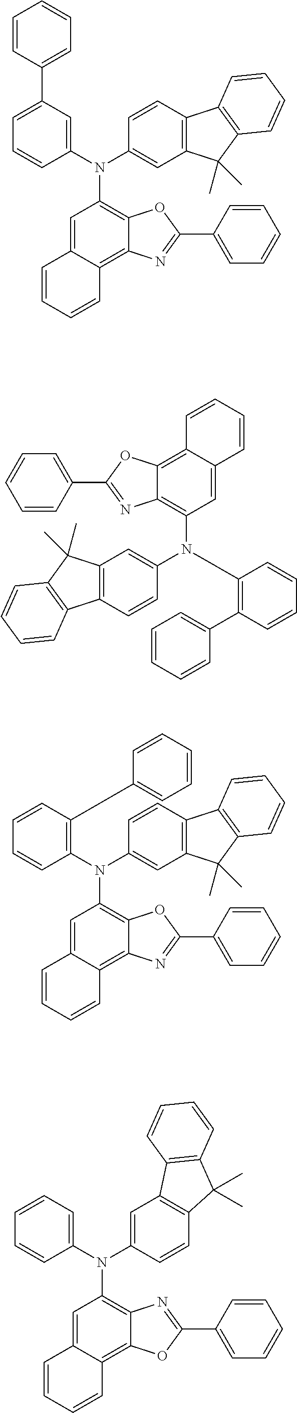 [SMILES: CC1(C)c2ccccc2-c2ccc(N(c3cccc(-c4ccccc4)c3)c3cc4ccccc4c4nc(-c5ccccc5)oc34)cc21 ; CC1(C)c2ccccc2-c2ccc(N(c3ccccc3-c3ccccc3)c3cc4ccccc4c4oc(-c5ccccc5)nc34)cc21 ; CC1(C)c2ccccc2-c2ccc(N(c3ccccc3-c3ccccc3)c3cc4ccccc4c4nc(-c5ccccc5)oc34)cc21 ; CC1(C)c2ccccc2-c2cc(N(c3ccccc3)c3cc4ccccc4c4oc(-c5ccccc5)nc34)ccc21]

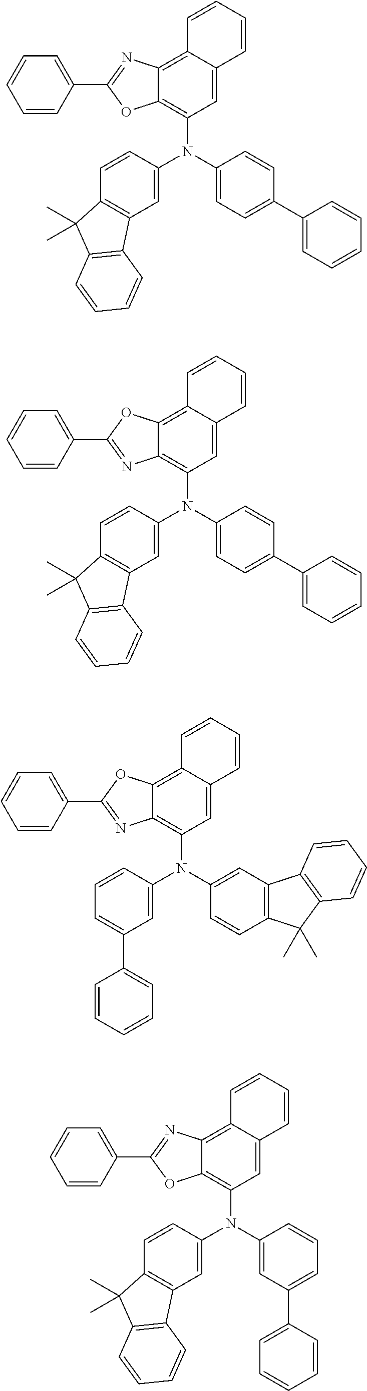 [SMILES: CC1(C)c2ccccc2-c2cc(N(c3ccc(-c4ccccc4)cc3)c3cc4ccccc4c4nc(-c5ccccc5)oc34)ccc21 ; CC1(C)c2ccccc2-c2cc(N(c3ccc(-c4ccccc4)cc3)c3cc4ccccc4c4oc(-c5ccccc5)nc34)ccc21 ; CC1(C)c2ccccc2-c2cc(N(c3cccc(-c4ccccc4)c3)c3cc4ccccc4c4oc(-c5ccccc5)nc34)ccc21 ; CC1(C)c2ccccc2-c2cc(N(c3cccc(-c4ccccc4)c3)c3cc4ccccc4c4nc(-c5ccccc5)oc34)ccc21]

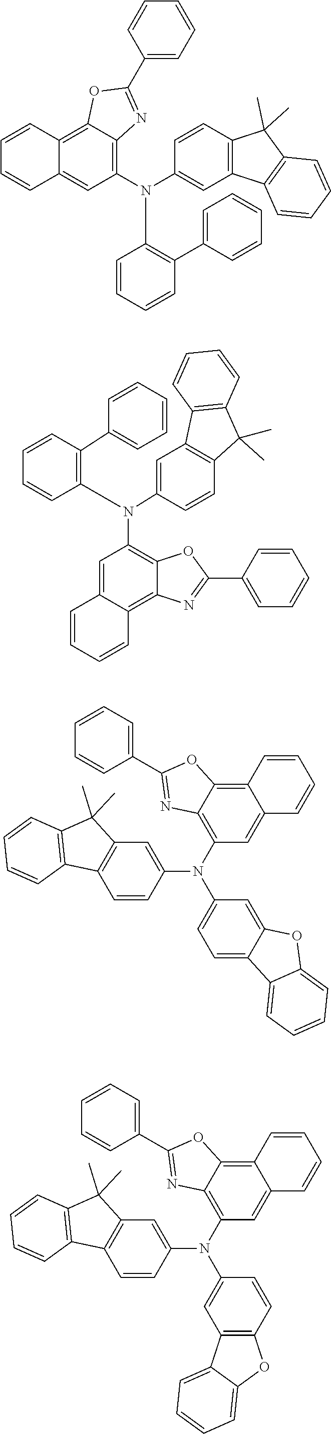

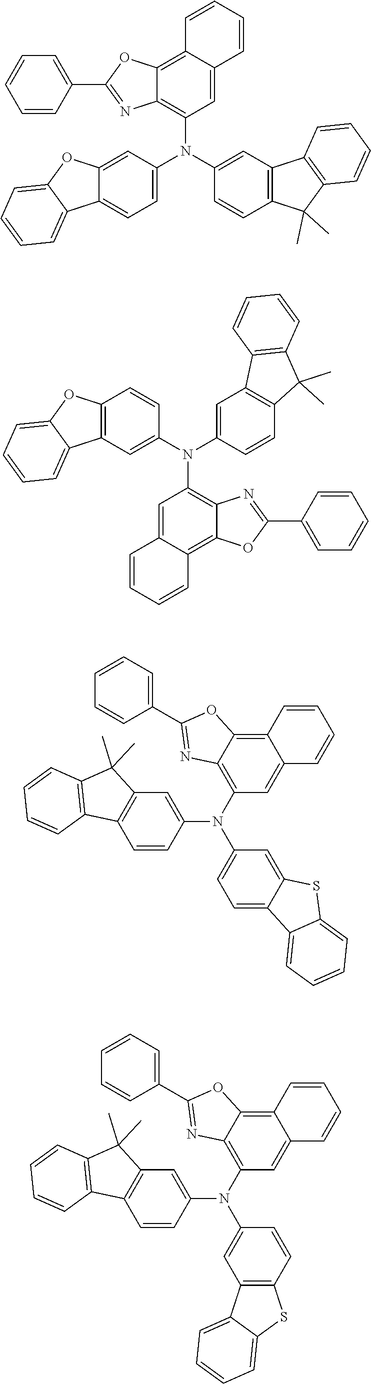 [SMILES: CC1(C)c2ccccc2-c2cc(N(c3ccc4c(c3)oc3ccccc34)c3cc4ccccc4c4oc(-c5ccccc5)nc34)ccc21 ; CC1(C)c2ccccc2-c2cc(N(c3ccc4oc5ccccc5c4c3)c3cc4ccccc4c4oc(-c5ccccc5)nc34)ccc21 ; CC1(C)c2ccccc2-c2ccc(N(c3ccc4c(c3)sc3ccccc34)c3cc4ccccc4c4oc(-c5ccccc5)nc34)cc21 ; CC1(C)c2ccccc2-c2ccc(N(c3ccc4sc5ccccc5c4c3)c3cc4ccccc4c4oc(-c5ccccc5)nc34)cc21]

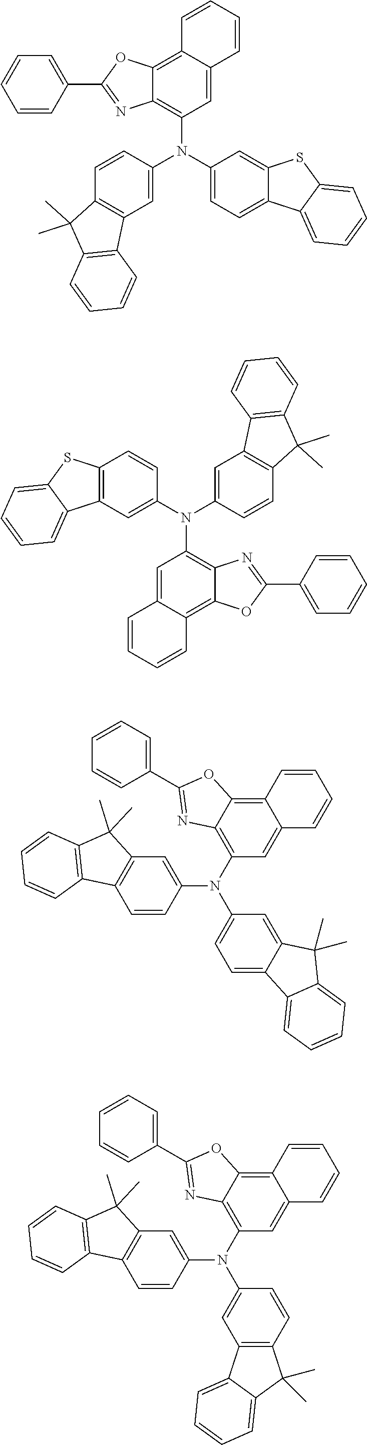 [SMILES: CC1(C)c2ccccc2-c2cc(N(c3ccc4c(c3)sc3ccccc34)c3cc4ccccc4c4oc(-c5ccccc5)nc34)ccc21 ; CC1(C)c2ccccc2-c2cc(N(c3ccc4sc5ccccc5c4c3)c3cc4ccccc4c4oc(-c5ccccc5)nc34)ccc21 ; CC1(C)c2ccccc2-c2ccc(N(c3ccc4c(c3)C(C)(C)c3ccccc3-4)c3cc4ccccc4c4oc(-c5ccccc5)nc34)cc21 ; CC1(C)c2ccccc2-c2cc(N(c3ccc4c(c3)C(C)(C)c3ccccc3-4)c3cc4ccccc4c4oc(-c5ccccc5)nc34)ccc21]

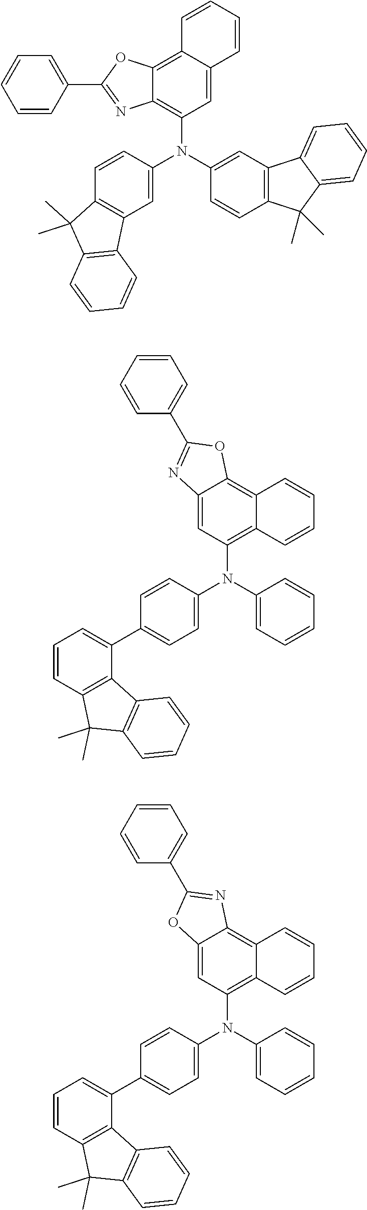 [SMILES: CC1(C)c2ccccc2-c2cc(N(c3ccc4c(c3)-c3ccccc3C4(C)C)c3cc4ccccc4c4oc(-c5ccccc5)nc34)ccc21 ; CC1(C)c2ccccc2-c2c(-c3ccc(N(c4ccccc4)c4cc5nc(-c6ccccc6)oc5c5ccccc45)cc3)cccc21 ; CC1(C)c2ccccc2-c2c(-c3ccc(N(c4ccccc4)c4cc5oc(-c6ccccc6)nc5c5ccccc45)cc3)cccc21]

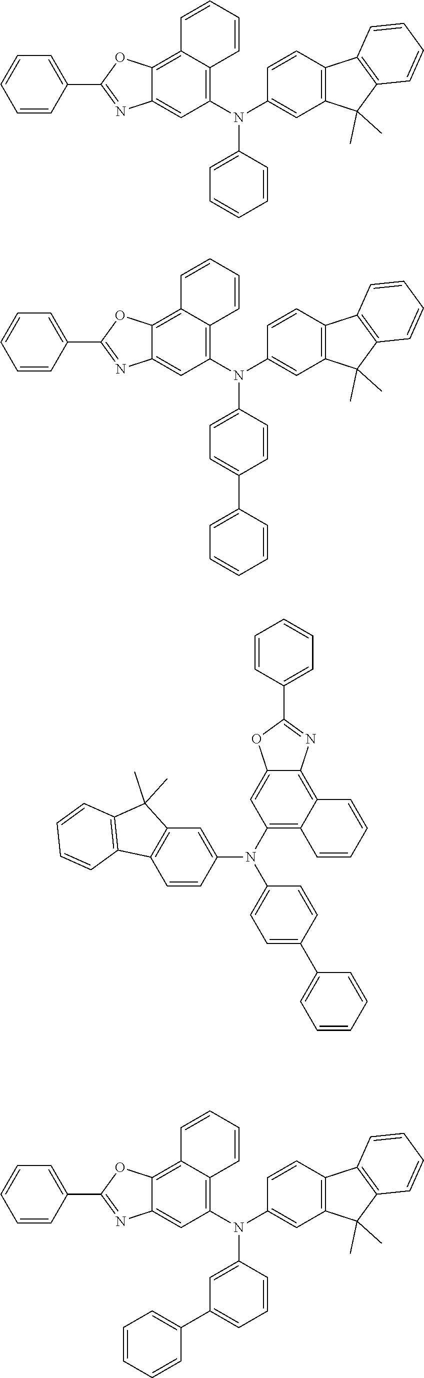 [SMILES: CC1(C)c2ccccc2-c2ccc(N(c3ccccc3)c3cc4nc(-c5ccccc5)oc4c4ccccc34)cc21 ; CC1(C)c2ccccc2-c2ccc(N(c3ccc(-c4ccccc4)cc3)c3cc4nc(-c5ccccc5)oc4c4ccccc34)cc21 ; CC1(C)c2ccccc2-c2ccc(N(c3ccc(-c4ccccc4)cc3)c3cc4oc(-c5ccccc5)nc4c4ccccc34)cc21 ; CC1(C)c2ccccc2-c2ccc(N(c3cccc(-c4ccccc4)c3)c3cc4nc(-c5ccccc5)oc4c4ccccc34)cc21]

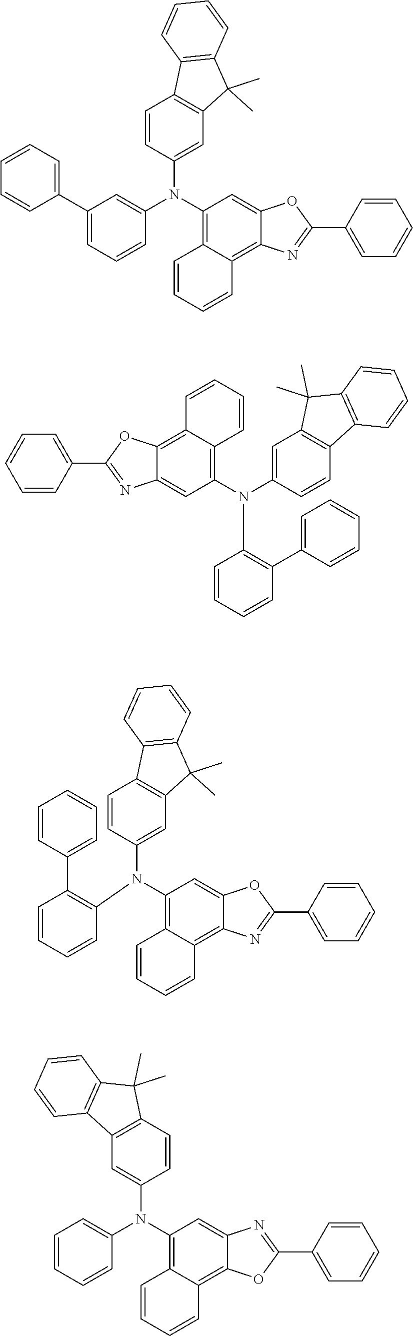 [SMILES: CC1(C)c2ccccc2-c2ccc(N(c3cccc(-c4ccccc4)c3)c3cc4oc(-c5ccccc5)nc4c4ccccc34)cc21 ; CC1(C)c2ccccc2-c2ccc(N(c3ccccc3-c3ccccc3)c3cc4nc(-c5ccccc5)oc4c4ccccc34)cc21 ; CC1(C)c2ccccc2-c2ccc(N(c3ccccc3-c3ccccc3)c3cc4oc(-c5ccccc5)nc4c4ccccc34)cc21 ; CC1(C)c2ccccc2-c2cc(N(c3ccccc3)c3cc4nc(-c5ccccc5)oc4c4ccccc34)ccc21]

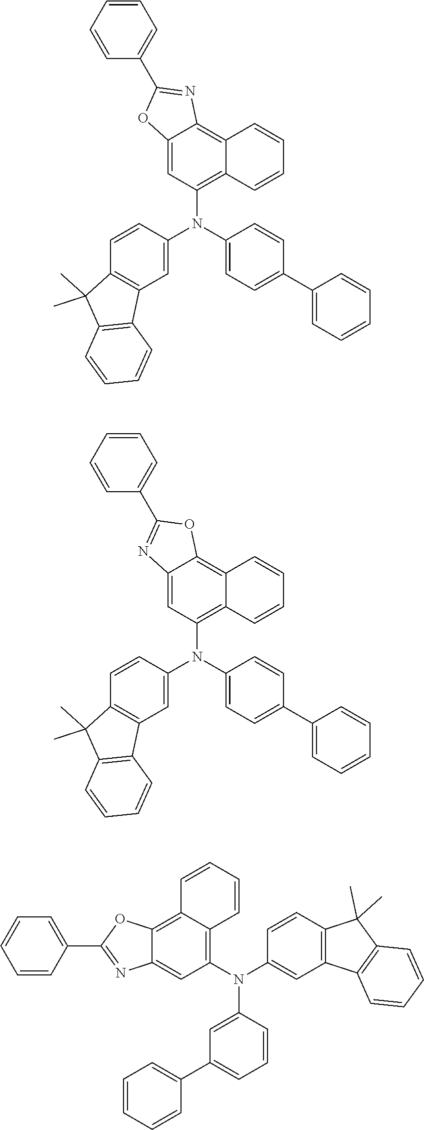 [SMILES: CC1(C)c2ccccc2-c2cc(N(c3ccc(-c4ccccc4)cc3)c3cc4oc(-c5ccccc5)nc4c4ccccc34)ccc21 ; CC1(C)c2ccccc2-c2cc(N(c3ccc(-c4ccccc4)cc3)c3cc4nc(-c5ccccc5)oc4c4ccccc34)ccc21 ; CC1(C)c2ccccc2-c2cc(N(c3cccc(-c4ccccc4)c3)c3cc4nc(-c5ccccc5)oc4c4ccccc34)ccc21]

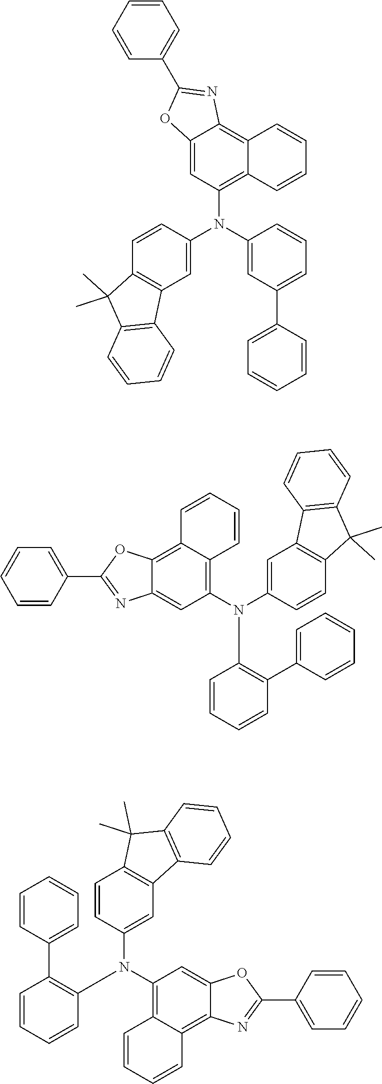 [SMILES: CC1(C)c2ccccc2-c2cc(N(c3cccc(-c4ccccc4)c3)c3cc4oc(-c5ccccc5)nc4c4ccccc34)ccc21 ; CC1(C)c2ccccc2-c2cc(N(c3ccccc3-c3ccccc3)c3cc4nc(-c5ccccc5)oc4c4ccccc34)ccc21 ; CC1(C)c2ccccc2-c2cc(N(c3ccccc3-c3ccccc3)c3cc4oc(-c5ccccc5)nc4c4ccccc34)ccc21]

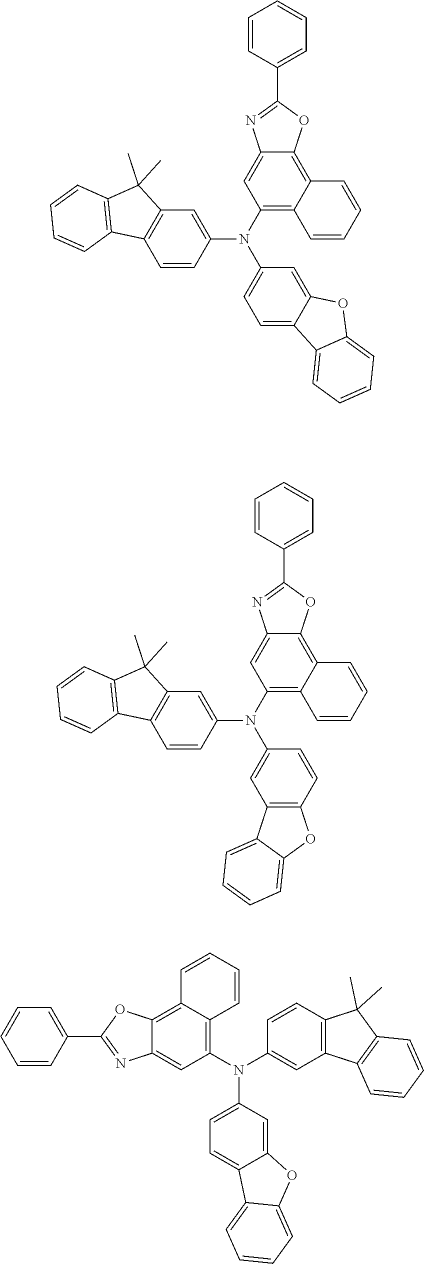 [SMILES: CC1(C)c2ccccc2-c2ccc(N(c3ccc4c(c3)oc3ccccc34)c3cc4nc(-c5ccccc5)oc4c4ccccc34)cc21 ; CC1(C)c2ccccc2-c2ccc(N(c3ccc4oc5ccccc5c4c3)c3cc4nc(-c5ccccc5)oc4c4ccccc34)cc21 ; CC1(C)c2ccccc2-c2cc(N(c3ccc4c(c3)oc3ccccc34)c3cc4nc(-c5ccccc5)oc4c4ccccc34)ccc21]

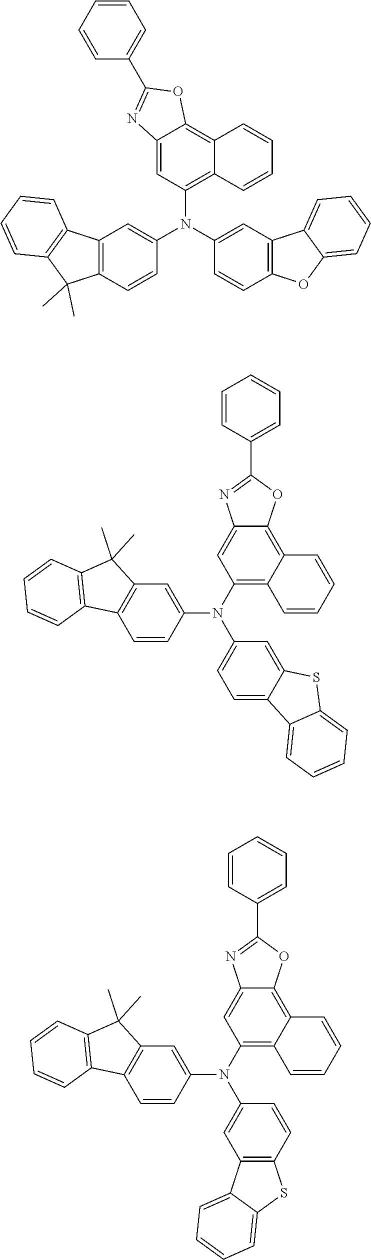 [SMILES: CC1(C)c2ccccc2-c2cc(N(c3ccc4oc5ccccc5c4c3)c3cc4nc(-c5ccccc5)oc4c4ccccc34)ccc21 ; CC1(C)c2ccccc2-c2ccc(N(c3ccc4c(c3)sc3ccccc34)c3cc4nc(-c5ccccc5)oc4c4ccccc34)cc21 ; CC1(C)c2ccccc2-c2ccc(N(c3ccc4sc5ccccc5c4c3)c3cc4nc(-c5ccccc5)oc4c4ccccc34)cc21]

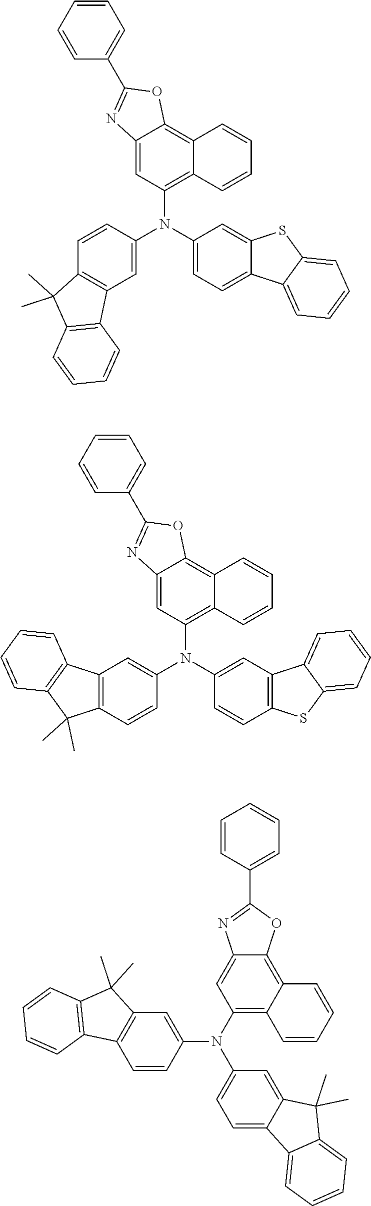 [SMILES: CC1(C)c2ccccc2-c2cc(N(c3ccc4c(c3)sc3ccccc34)c3cc4nc(-c5ccccc5)oc4c4ccccc34)ccc21 ; CC1(C)c2ccccc2-c2cc(N(c3ccc4sc5ccccc5c4c3)c3cc4nc(-c5ccccc5)oc4c4ccccc34)ccc21 ; CC1(C)c2ccccc2-c2ccc(N(c3ccc4c(c3)C(C)(C)c3ccccc3-4)c3cc4nc(-c5ccccc5)oc4c4ccccc34)cc21]

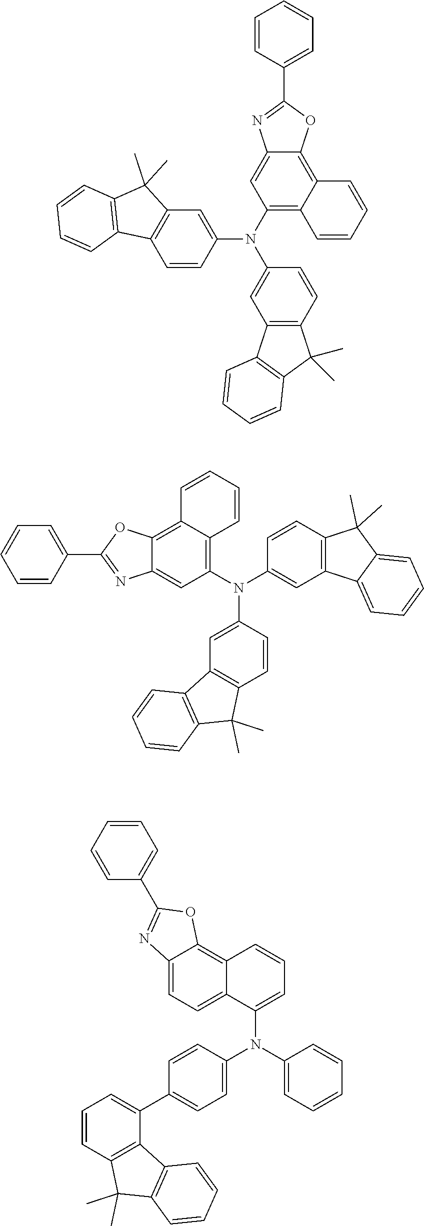 [SMILES: CC1(C)c2ccccc2-c2cc(N(c3ccc4c(c3)C(C)(C)c3ccccc3-4)c3cc4nc(-c5ccccc5)oc4c4ccccc34)ccc21 ; CC1(C)c2ccccc2-c2cc(N(c3ccc4c(c3)-c3ccccc3C4(C)C)c3cc4nc(-c5ccccc5)oc4c4ccccc34)ccc21 ; CC1(C)c2ccccc2-c2c(-c3ccc(N(c4ccccc4)c4cccc5c4ccc4nc(-c6ccccc6)oc45)cc3)cccc21]

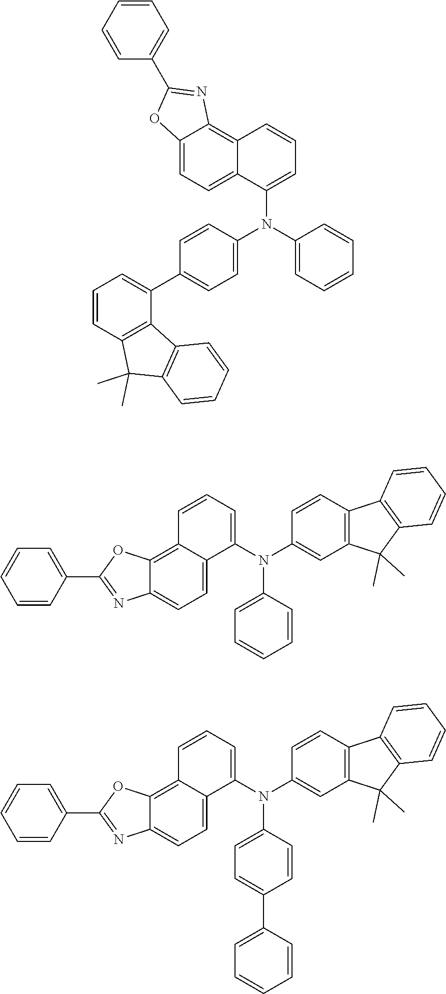 [SMILES: CC1(C)c2ccccc2-c2c(-c3ccc(N(c4ccccc4)c4cccc5c4ccc4oc(-c6ccccc6)nc45)cc3)cccc21 ; CC1(C)c2ccccc2-c2ccc(N(c3ccccc3)c3cccc4c3ccc3nc(-c5ccccc5)oc34)cc21 ; CC1(C)c2ccccc2-c2ccc(N(c3ccc(-c4ccccc4)cc3)c3cccc4c3ccc3nc(-c5ccccc5)oc34)cc21]

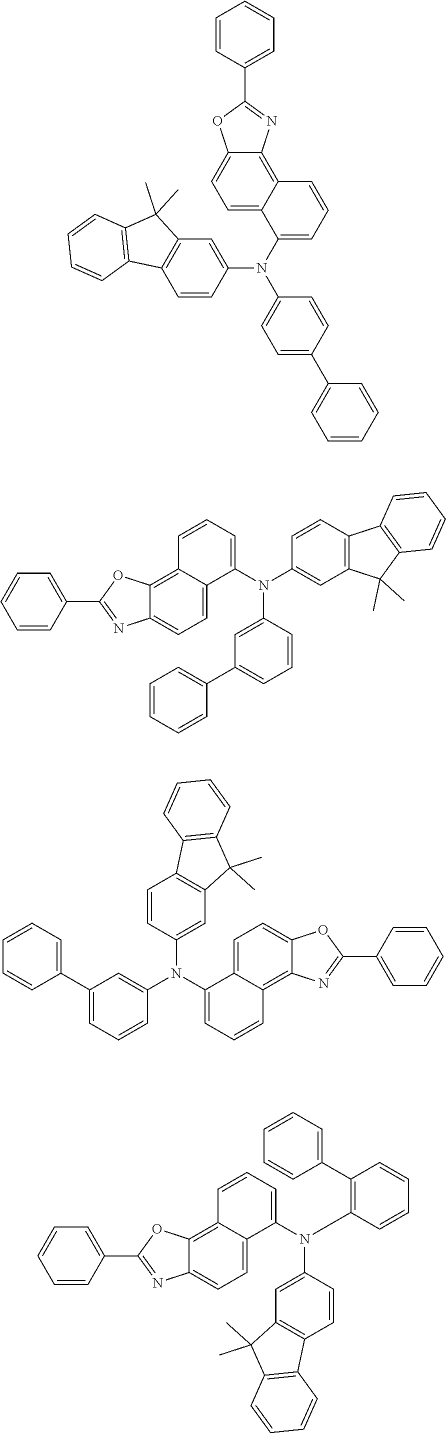 [SMILES: CC1(C)c2ccccc2-c2ccc(N(c3ccc(-c4ccccc4)cc3)c3cccc4c3ccc3oc(-c5ccccc5)nc34)cc21 ; CC1(C)c2ccccc2-c2ccc(N(c3cccc(-c4ccccc4)c3)c3cccc4c3ccc3nc(-c5ccccc5)oc34)cc21 ; CC1(C)c2ccccc2-c2ccc(N(c3cccc(-c4ccccc4)c3)c3cccc4c3ccc3oc(-c5ccccc5)nc34)cc21 ; CC1(C)c2ccccc2-c2ccc(N(c3ccccc3-c3ccccc3)c3cccc4c3ccc3nc(-c5ccccc5)oc34)cc21]

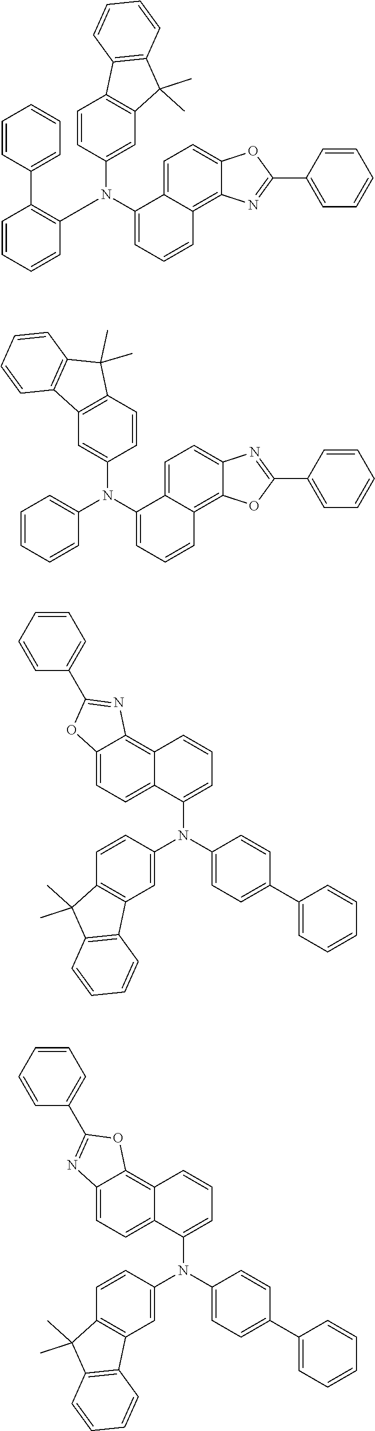 [SMILES: CC1(C)c2ccccc2-c2ccc(N(c3ccccc3-c3ccccc3)c3cccc4c3ccc3oc(-c5ccccc5)nc34)cc21 ; CC1(C)c2ccccc2-c2cc(N(c3ccccc3)c3cccc4c3ccc3nc(-c5ccccc5)oc34)ccc21 ; CC1(C)c2ccccc2-c2cc(N(c3ccc(-c4ccccc4)cc3)c3cccc4c3ccc3oc(-c5ccccc5)nc34)ccc21 ; CC1(C)c2ccccc2-c2cc(N(c3ccc(-c4ccccc4)cc3)c3cccc4c3ccc3nc(-c5ccccc5)oc34)ccc21]

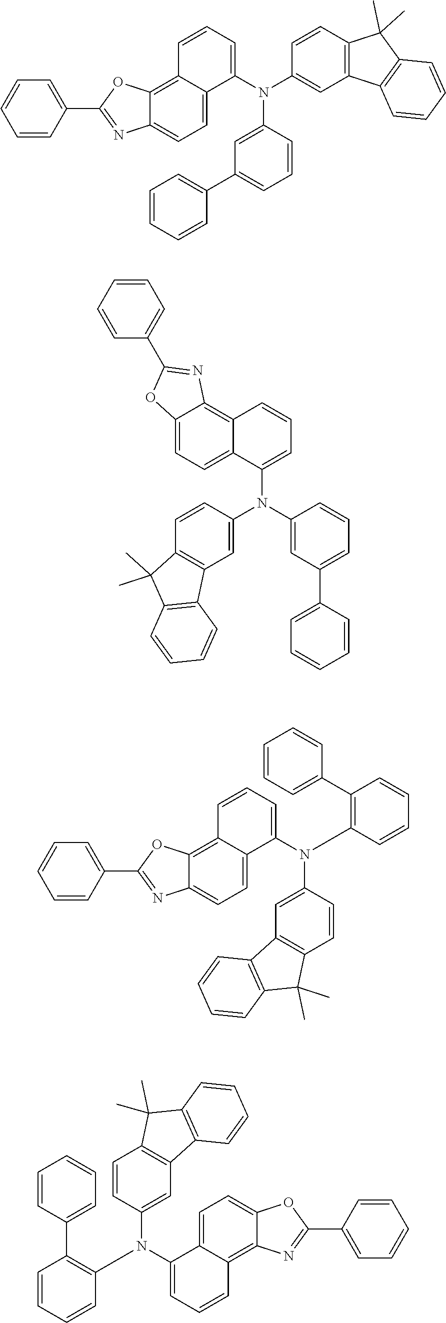 [SMILES: CC1(C)c2ccccc2-c2cc(N(c3cccc(-c4ccccc4)c3)c3cccc4c3ccc3nc(-c5ccccc5)oc34)ccc21 ; CC1(C)c2ccccc2-c2cc(N(c3cccc(-c4ccccc4)c3)c3cccc4c3ccc3oc(-c5ccccc5)nc34)ccc21 ; CC1(C)c2ccccc2-c2cc(N(c3ccccc3-c3ccccc3)c3cccc4c3ccc3nc(-c5ccccc5)oc34)ccc21 ; CC1(C)c2ccccc2-c2cc(N(c3ccccc3-c3ccccc3)c3cccc4c3ccc3oc(-c5ccccc5)nc34)ccc21]

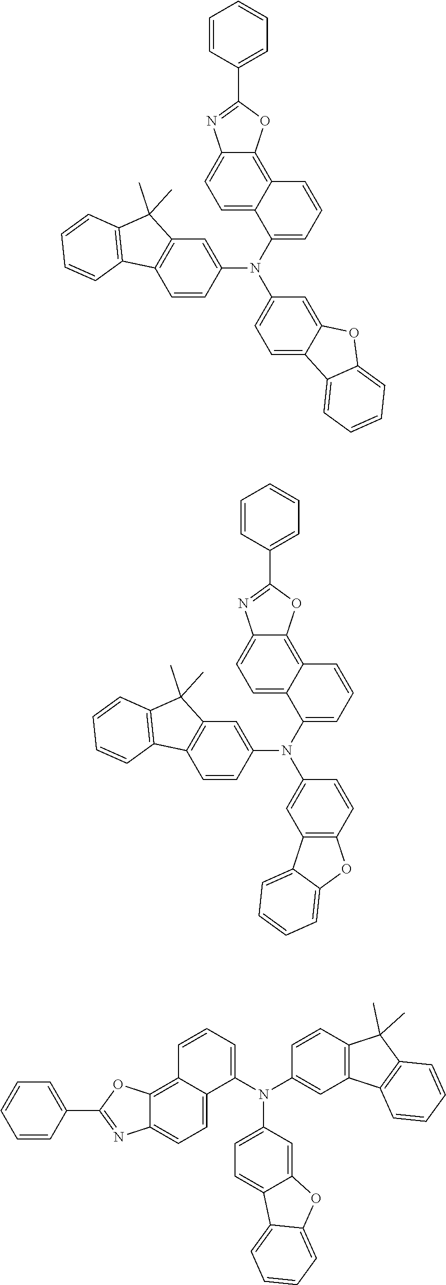 [SMILES: CC1(C)c2ccccc2-c2ccc(N(c3ccc4c(c3)oc3ccccc34)c3cccc4c3ccc3nc(-c5ccccc5)oc34)cc21 ; CC1(C)c2ccccc2-c2ccc(N(c3ccc4oc5ccccc5c4c3)c3cccc4c3ccc3nc(-c5ccccc5)oc34)cc21 ; CC1(C)c2ccccc2-c2cc(N(c3ccc4c(c3)oc3ccccc34)c3cccc4c3ccc3nc(-c5ccccc5)oc34)ccc21]

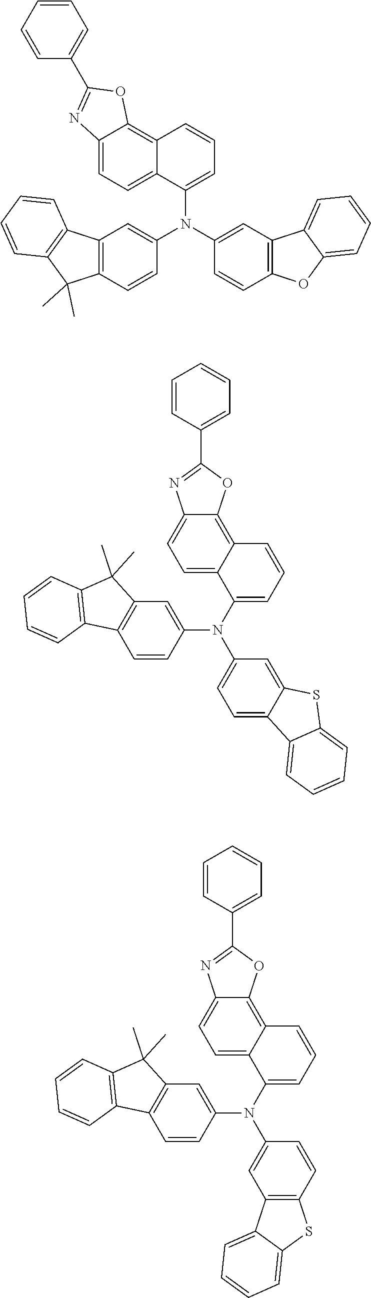 [SMILES: CC1(C)c2ccccc2-c2cc(N(c3ccc4oc5ccccc5c4c3)c3cccc4c3ccc3nc(-c5ccccc5)oc34)ccc21 ; CC1(C)c2ccccc2-c2ccc(N(c3ccc4c(c3)sc3ccccc34)c3cccc4c3ccc3nc(-c5ccccc5)oc34)cc21 ; CC1(C)c2ccccc2-c2ccc(N(c3ccc4sc5ccccc5c4c3)c3cccc4c3ccc3nc(-c5ccccc5)oc34)cc21]

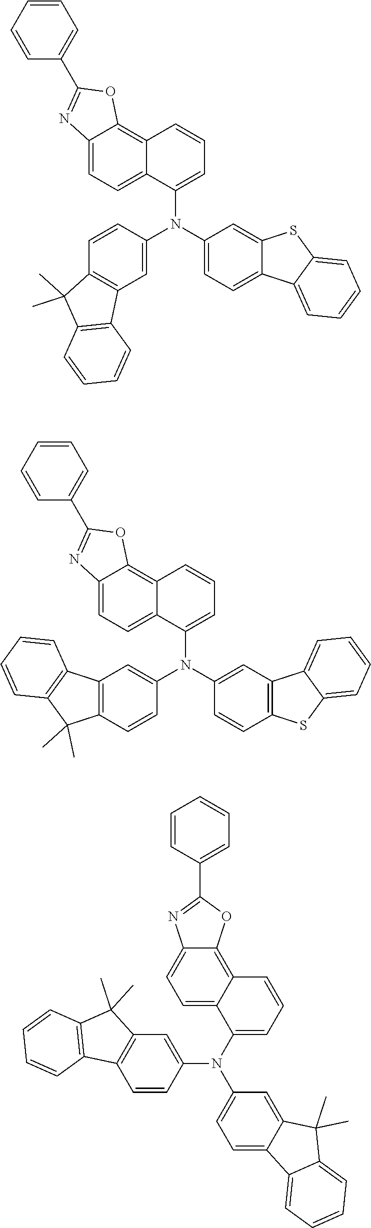 [SMILES: CC1(C)c2ccccc2-c2cc(N(c3ccc4c(c3)sc3ccccc34)c3cccc4c3ccc3nc(-c5ccccc5)oc34)ccc21 ; CC1(C)c2ccccc2-c2cc(N(c3ccc4sc5ccccc5c4c3)c3cccc4c3ccc3nc(-c5ccccc5)oc34)ccc21 ; CC1(C)c2ccccc2-c2ccc(N(c3ccc4c(c3)C(C)(C)c3ccccc3-4)c3cccc4c3ccc3nc(-c5ccccc5)oc34)cc21]

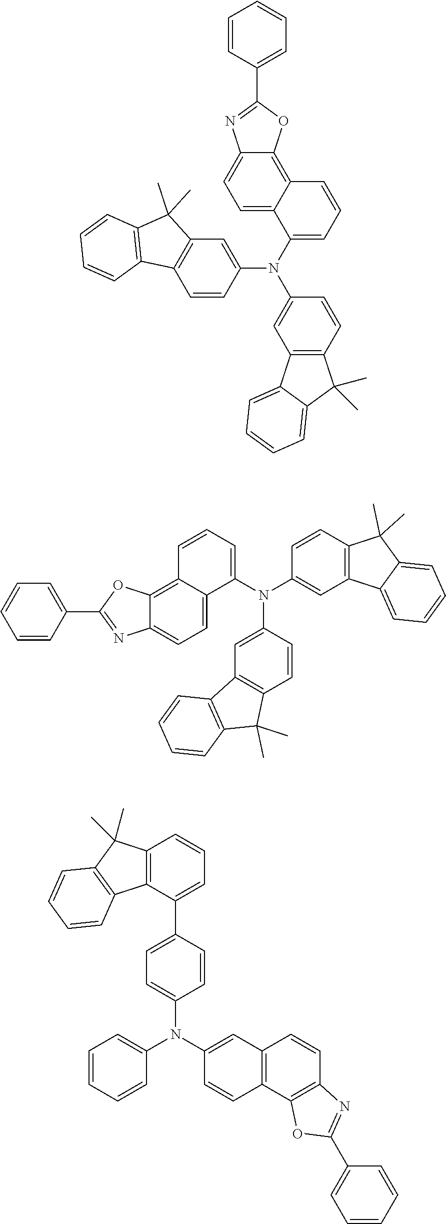 [SMILES: CC1(C)c2ccccc2-c2cc(N(c3ccc4c(c3)C(C)(C)c3ccccc3-4)c3cccc4c3ccc3nc(-c5ccccc5)oc34)ccc21 ; CC1(C)c2ccccc2-c2cc(N(c3ccc4c(c3)-c3ccccc3C4(C)C)c3cccc4c3ccc3nc(-c5ccccc5)oc34)ccc21 ; CC1(C)c2ccccc2-c2c(-c3ccc(N(c4ccccc4)c4ccc5c(ccc6nc(-c7ccccc7)oc65)c4)cc3)cccc21]

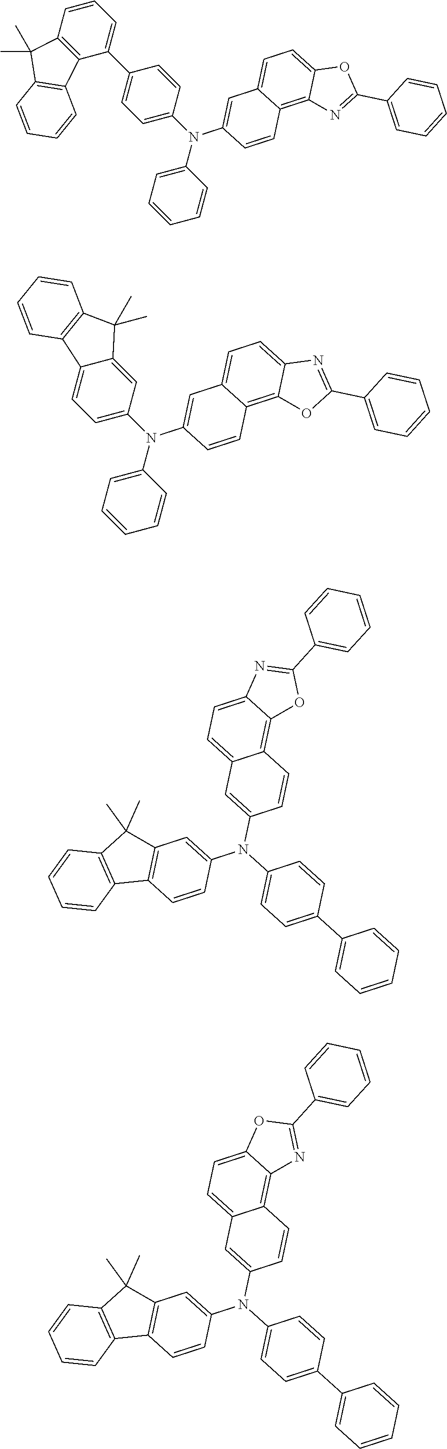 [SMILES: CC1(C)c2ccccc2-c2c(-c3ccc(N(c4ccccc4)c4ccc5c(ccc6oc(-c7ccccc7)nc65)c4)cc3)cccc21 ; CC1(C)c2ccccc2-c2ccc(N(c3ccccc3)c3ccc4c(ccc5nc(-c6ccccc6)oc54)c3)cc21 ; CC1(C)c2ccccc2-c2ccc(N(c3ccc(-c4ccccc4)cc3)c3ccc4c(ccc5nc(-c6ccccc6)oc54)c3)cc21 ; CC1(C)c2ccccc2-c2ccc(N(c3ccc(-c4ccccc4)cc3)c3ccc4c(ccc5oc(-c6ccccc6)nc54)c3)cc21]

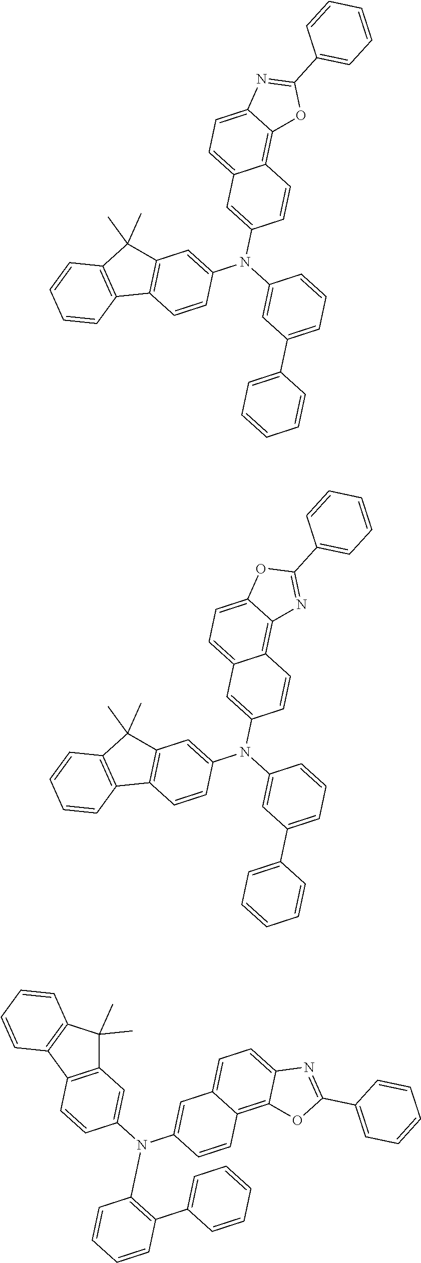 [SMILES: CC1(C)c2ccccc2-c2ccc(N(c3cccc(-c4ccccc4)c3)c3ccc4c(ccc5nc(-c6ccccc6)oc54)c3)cc21 ; CC1(C)c2ccccc2-c2ccc(N(c3cccc(-c4ccccc4)c3)c3ccc4c(ccc5oc(-c6ccccc6)nc54)c3)cc21 ; CC1(C)c2ccccc2-c2ccc(N(c3ccc4c(ccc5nc(-c6ccccc6)oc54)c3)c3ccccc3-c3ccccc3)cc21]

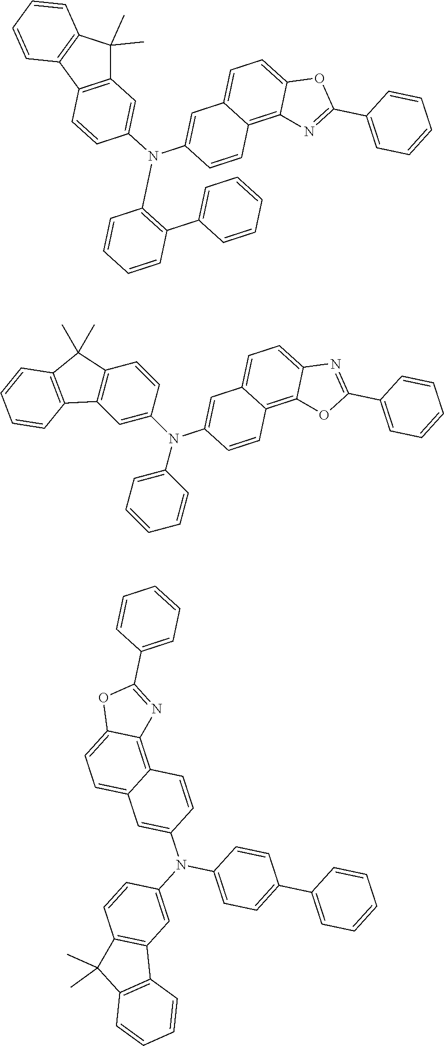 [SMILES: CC1(C)c2ccccc2-c2ccc(N(c3ccc4c(ccc5oc(-c6ccccc6)nc54)c3)c3ccccc3-c3ccccc3)cc21 ; CC1(C)c2ccccc2-c2cc(N(c3ccccc3)c3ccc4c(ccc5nc(-c6ccccc6)oc54)c3)ccc21 ; CC1(C)c2ccccc2-c2cc(N(c3ccc(-c4ccccc4)cc3)c3ccc4c(ccc5oc(-c6ccccc6)nc54)c3)ccc21]

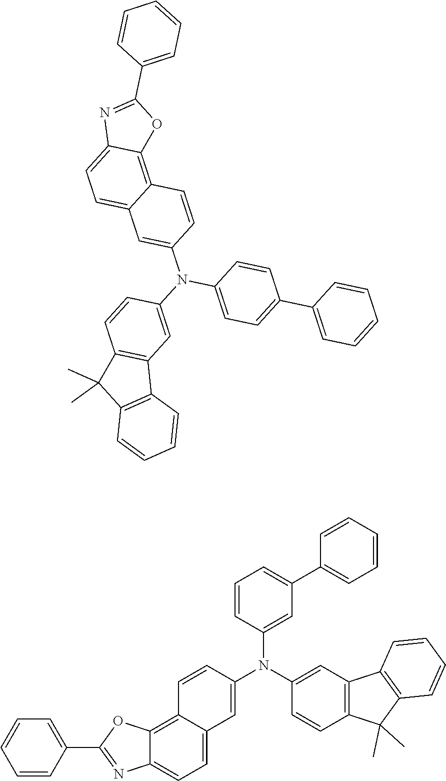 [SMILES: CC1(C)c2ccccc2-c2cc(N(c3ccc(-c4ccccc4)cc3)c3ccc4c(ccc5nc(-c6ccccc6)oc54)c3)ccc21 ; CC1(C)c2ccccc2-c2cc(N(c3cccc(-c4ccccc4)c3)c3ccc4c(ccc5nc(-c6ccccc6)oc54)c3)ccc21]

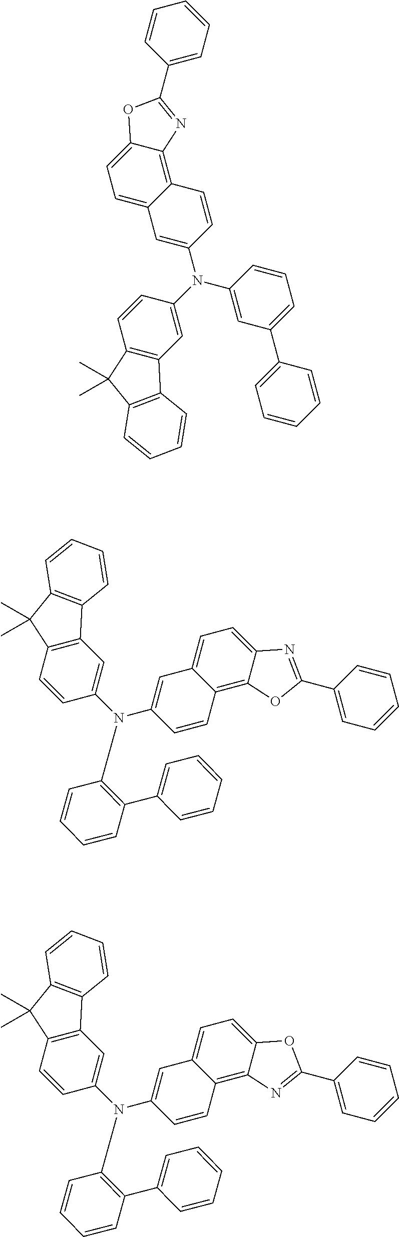 [SMILES: CC1(C)c2ccccc2-c2cc(N(c3cccc(-c4ccccc4)c3)c3ccc4c(ccc5oc(-c6ccccc6)nc54)c3)ccc21 ; CC1(C)c2ccccc2-c2cc(N(c3ccc4c(ccc5nc(-c6ccccc6)oc54)c3)c3ccccc3-c3ccccc3)ccc21 ; CC1(C)c2ccccc2-c2cc(N(c3ccc4c(ccc5oc(-c6ccccc6)nc54)c3)c3ccccc3-c3ccccc3)ccc21]

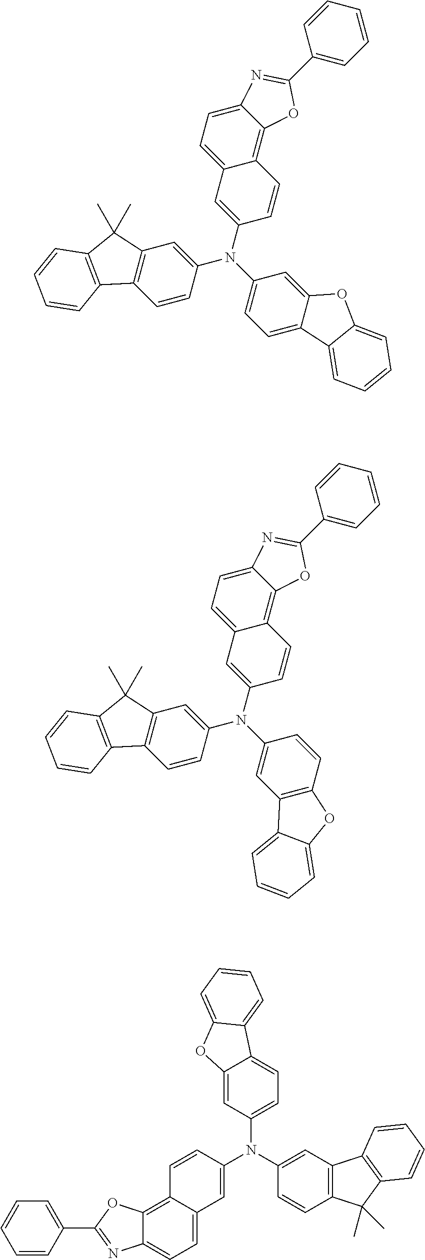 [SMILES: CC1(C)c2ccccc2-c2ccc(N(c3ccc4c(ccc5nc(-c6ccccc6)oc54)c3)c3ccc4c(c3)oc3ccccc34)cc21 ; CC1(C)c2ccccc2-c2ccc(N(c3ccc4c(ccc5nc(-c6ccccc6)oc54)c3)c3ccc4oc5ccccc5c4c3)cc21 ; CC1(C)c2ccccc2-c2cc(N(c3ccc4c(ccc5nc(-c6ccccc6)oc54)c3)c3ccc4c(c3)oc3ccccc34)ccc21]

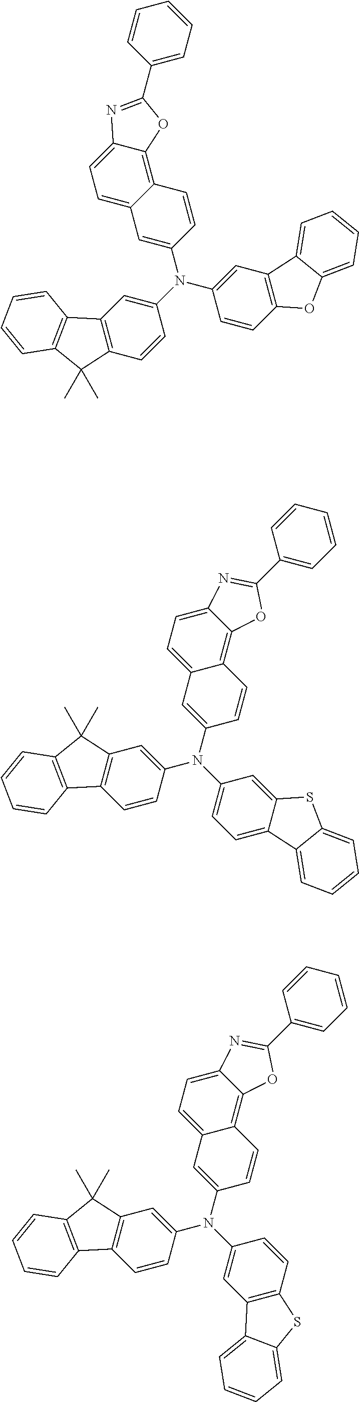 [SMILES: CC1(C)c2ccccc2-c2cc(N(c3ccc4c(ccc5nc(-c6ccccc6)oc54)c3)c3ccc4oc5ccccc5c4c3)ccc21 ; CC1(C)c2ccccc2-c2ccc(N(c3ccc4c(ccc5nc(-c6ccccc6)oc54)c3)c3ccc4c(c3)sc3ccccc34)cc21 ; CC1(C)c2ccccc2-c2ccc(N(c3ccc4c(ccc5nc(-c6ccccc6)oc54)c3)c3ccc4sc5ccccc5c4c3)cc21]

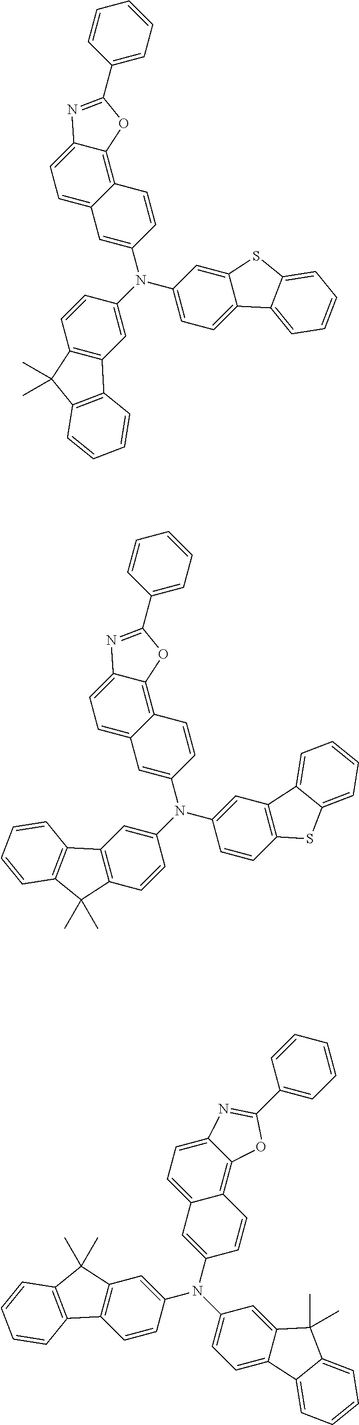 [SMILES: CC1(C)c2ccccc2-c2cc(N(c3ccc4c(ccc5nc(-c6ccccc6)oc54)c3)c3ccc4c(c3)sc3ccccc34)ccc21 ; CC1(C)c2ccccc2-c2cc(N(c3ccc4c(ccc5nc(-c6ccccc6)oc54)c3)c3ccc4sc5ccccc5c4c3)ccc21 ; CC1(C)c2ccccc2-c2ccc(N(c3ccc4c(c3)C(C)(C)c3ccccc3-4)c3ccc4c(ccc5nc(-c6ccccc6)oc54)c3)cc21]

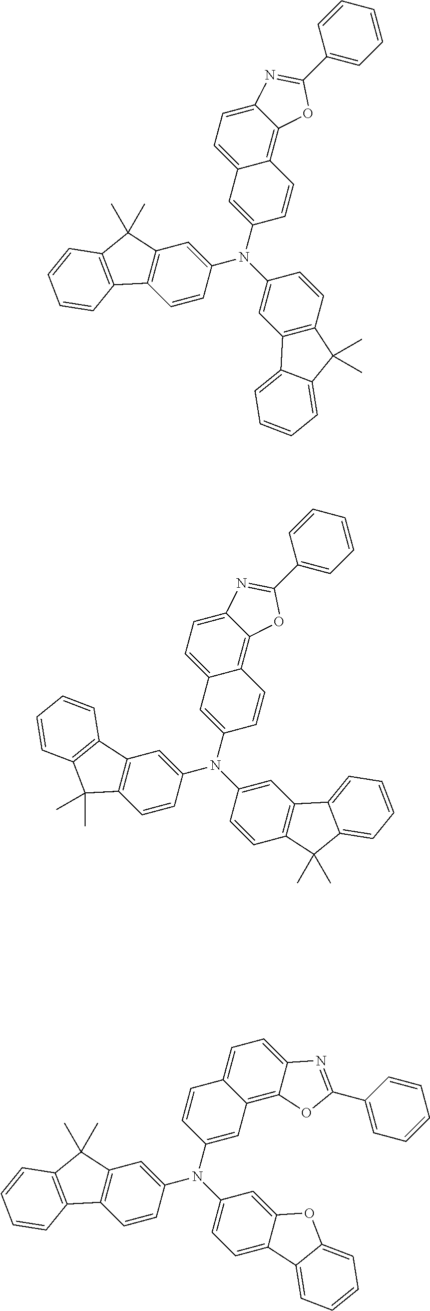 [SMILES: CC1(C)c2ccccc2-c2cc(N(c3ccc4c(c3)C(C)(C)c3ccccc3-4)c3ccc4c(ccc5nc(-c6ccccc6)oc54)c3)ccc21 ; CC1(C)c2ccccc2-c2cc(N(c3ccc4c(c3)-c3ccccc3C4(C)C)c3ccc4c(ccc5nc(-c6ccccc6)oc54)c3)ccc21 ; CC1(C)c2ccccc2-c2ccc(N(c3ccc4c(c3)oc3ccccc34)c3ccc4ccc5nc(-c6ccccc6)oc5c4c3)cc21]

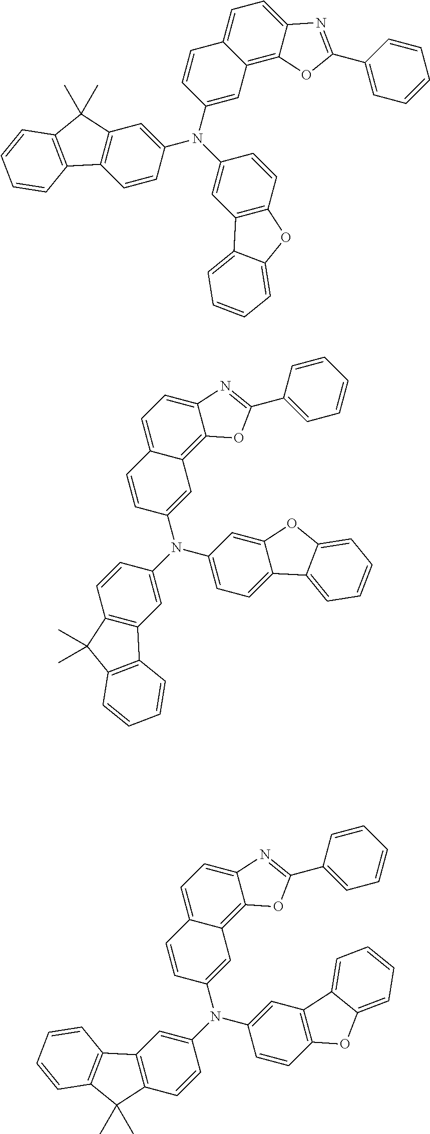 [SMILES: CC1(C)c2ccccc2-c2ccc(N(c3ccc4oc5ccccc5c4c3)c3ccc4ccc5nc(-c6ccccc6)oc5c4c3)cc21 ; CC1(C)c2ccccc2-c2cc(N(c3ccc4c(c3)oc3ccccc34)c3ccc4ccc5nc(-c6ccccc6)oc5c4c3)ccc21 ; CC1(C)c2ccccc2-c2cc(N(c3ccc4oc5ccccc5c4c3)c3ccc4ccc5nc(-c6ccccc6)oc5c4c3)ccc21]

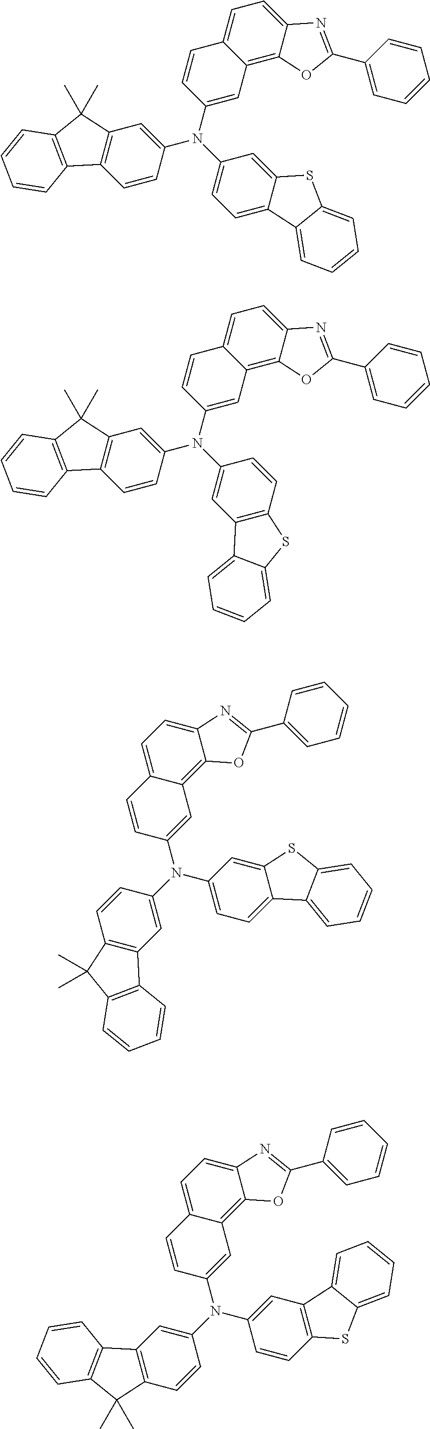 [SMILES: CC1(C)c2ccccc2-c2ccc(N(c3ccc4c(c3)sc3ccccc34)c3ccc4ccc5nc(-c6ccccc6)oc5c4c3)cc21 ; CC1(C)c2ccccc2-c2ccc(N(c3ccc4sc5ccccc5c4c3)c3ccc4ccc5nc(-c6ccccc6)oc5c4c3)cc21 ; CC1(C)c2ccccc2-c2cc(N(c3ccc4c(c3)sc3ccccc34)c3ccc4ccc5nc(-c6ccccc6)oc5c4c3)ccc21 ; CC1(C)c2ccccc2-c2cc(N(c3ccc4sc5ccccc5c4c3)c3ccc4ccc5nc(-c6ccccc6)oc5c4c3)ccc21]

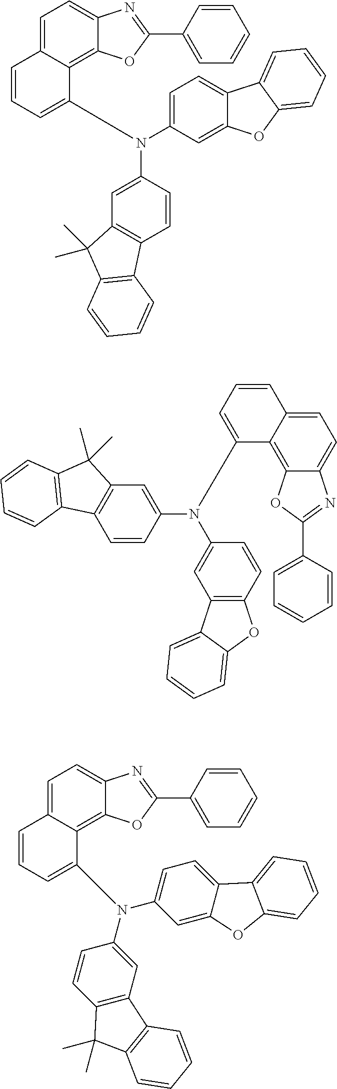 [SMILES: CC1(C)c2ccccc2-c2ccc(N(c3ccc4c(c3)oc3ccccc34)c3cccc4ccc5nc(-c6ccccc6)oc5c34)cc21 ; CC1(C)c2ccccc2-c2ccc(N(c3ccc4oc5ccccc5c4c3)c3cccc4ccc5nc(-c6ccccc6)oc5c34)cc21 ; CC1(C)c2ccccc2-c2cc(N(c3ccc4c(c3)oc3ccccc34)c3cccc4ccc5nc(-c6ccccc6)oc5c34)ccc21]

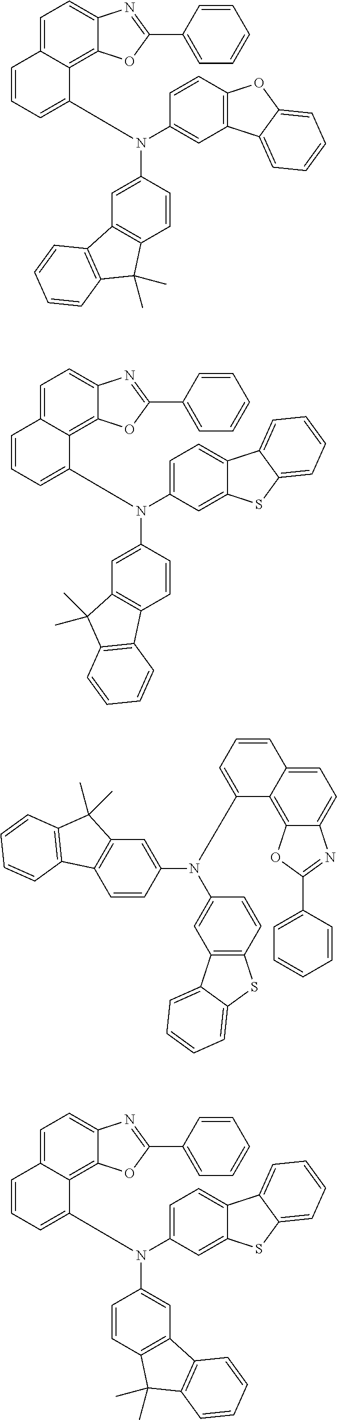 [SMILES: CC1(C)c2ccccc2-c2cc(N(c3ccc4oc5ccccc5c4c3)c3cccc4ccc5nc(-c6ccccc6)oc5c34)ccc21 ; CC1(C)c2ccccc2-c2ccc(N(c3ccc4c(c3)sc3ccccc34)c3cccc4ccc5nc(-c6ccccc6)oc5c34)cc21 ; CC1(C)c2ccccc2-c2ccc(N(c3ccc4sc5ccccc5c4c3)c3cccc4ccc5nc(-c6ccccc6)oc5c34)cc21 ; CC1(C)c2ccccc2-c2cc(N(c3ccc4c(c3)sc3ccccc34)c3cccc4ccc5nc(-c6ccccc6)oc5c34)ccc21]

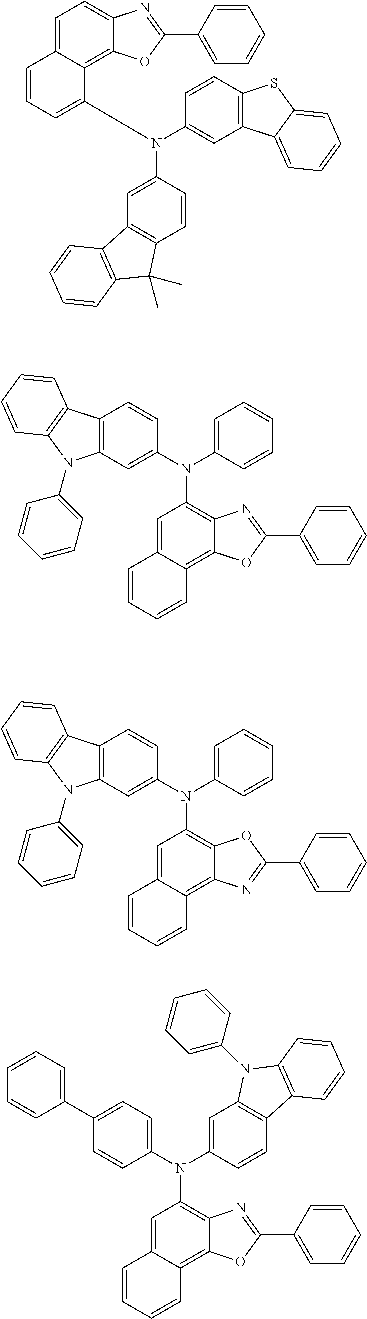 [SMILES: CC1(C)c2ccccc2-c2cc(N(c3ccc4sc5ccccc5c4c3)c3cccc4ccc5nc(-c6ccccc6)oc5c34)ccc21 ; c1ccc(-c2nc3c(N(c4ccccc4)c4ccc5c6ccccc6n(-c6ccccc6)c5c4)cc4ccccc4c3o2)cc1 ; c1ccc(-c2nc3c(o2)c(N(c2ccccc2)c2ccc4c5ccccc5n(-c5ccccc5)c4c2)cc2ccccc23)cc1 ; c1ccc(-c2ccc(N(c3ccc4c5ccccc5n(-c5ccccc5)c4c3)c3cc4ccccc4c4oc(-c5ccccc5)nc34)cc2)cc1]

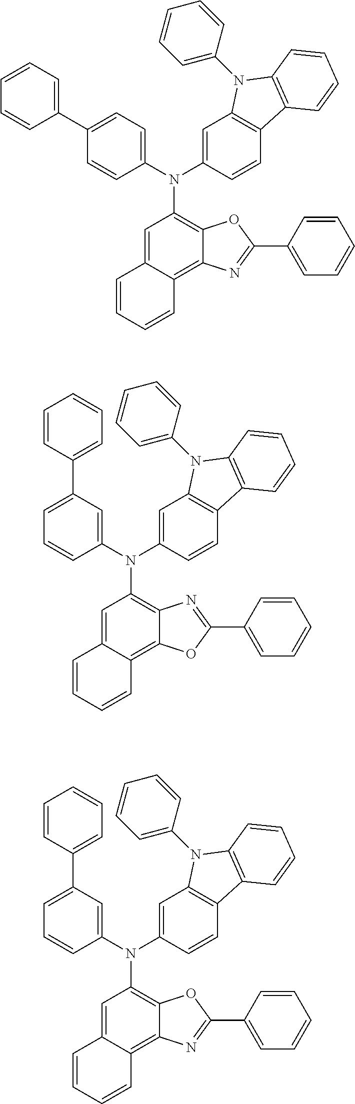 [SMILES: c1ccc(-c2ccc(N(c3ccc4c5ccccc5n(-c5ccccc5)c4c3)c3cc4ccccc4c4nc(-c5ccccc5)oc34)cc2)cc1 ; c1ccc(-c2cccc(N(c3ccc4c5ccccc5n(-c5ccccc5)c4c3)c3cc4ccccc4c4oc(-c5ccccc5)nc34)c2)cc1 ; c1ccc(-c2cccc(N(c3ccc4c5ccccc5n(-c5ccccc5)c4c3)c3cc4ccccc4c4nc(-c5ccccc5)oc34)c2)cc1]

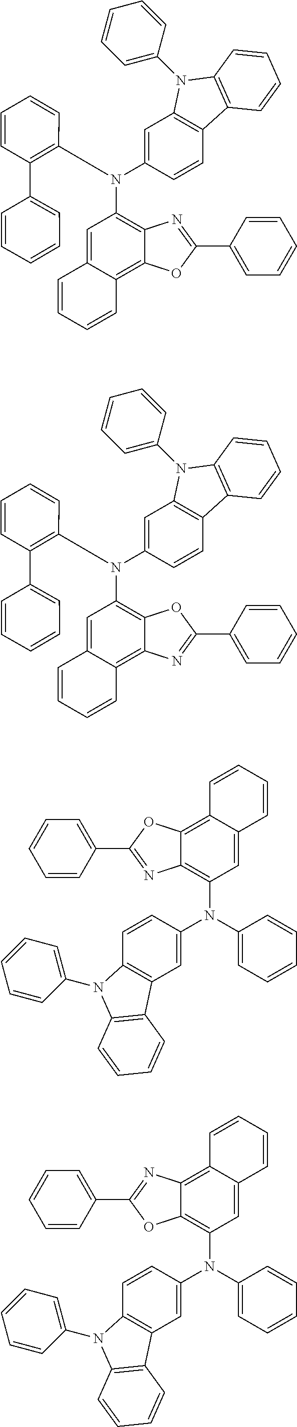 [SMILES: c1ccc(-c2nc3c(N(c4ccc5c6ccccc6n(-c6ccccc6)c5c4)c4ccccc4-c4ccccc4)cc4ccccc4c3o2)cc1 ; c1ccc(-c2nc3c(o2)c(N(c2ccc4c5ccccc5n(-c5ccccc5)c4c2)c2ccccc2-c2ccccc2)cc2ccccc23)cc1 ; c1ccc(-c2nc3c(N(c4ccccc4)c4ccc5c(c4)c4ccccc4n5-c4ccccc4)cc4ccccc4c3o2)cc1 ; c1ccc(-c2nc3c(o2)c(N(c2ccccc2)c2ccc4c(c2)c2ccccc2n4-c2ccccc2)cc2ccccc23)cc1]

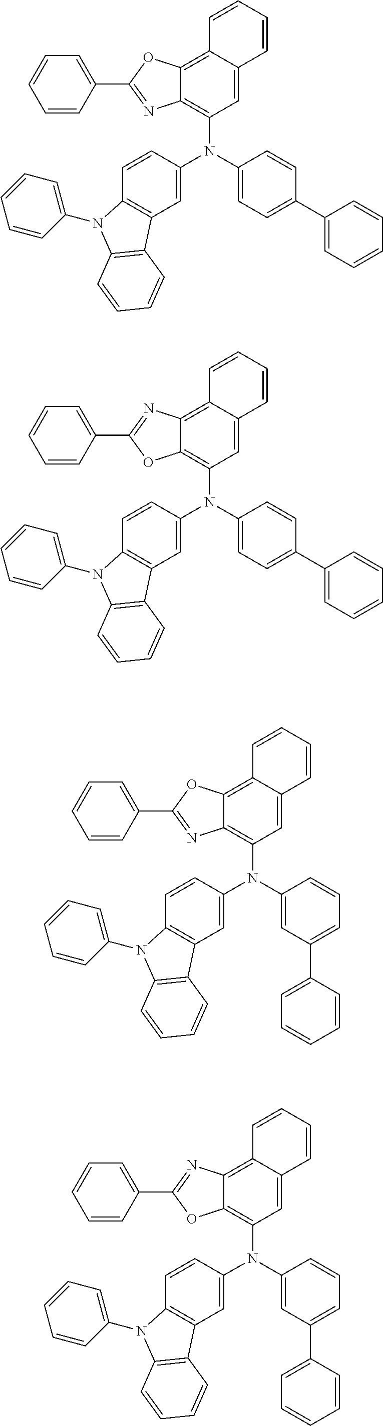 [SMILES: c1ccc(-c2ccc(N(c3ccc4c(c3)c3ccccc3n4-c3ccccc3)c3cc4ccccc4c4oc(-c5ccccc5)nc34)cc2)cc1 ; c1ccc(-c2ccc(N(c3ccc4c(c3)c3ccccc3n4-c3ccccc3)c3cc4ccccc4c4nc(-c5ccccc5)oc34)cc2)cc1 ; c1ccc(-c2cccc(N(c3ccc4c(c3)c3ccccc3n4-c3ccccc3)c3cc4ccccc4c4oc(-c5ccccc5)nc34)c2)cc1 ; c1ccc(-c2cccc(N(c3ccc4c(c3)c3ccccc3n4-c3ccccc3)c3cc4ccccc4c4nc(-c5ccccc5)oc34)c2)cc1]

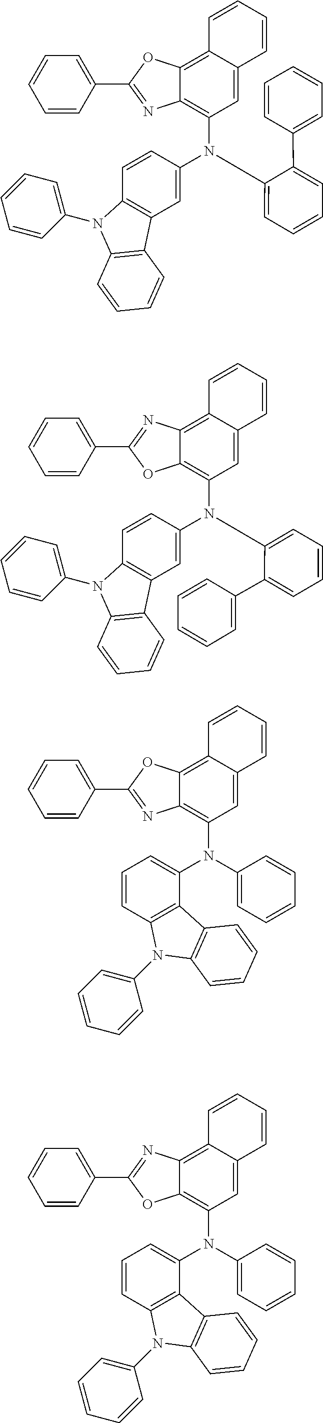 [SMILES: c1ccc(-c2nc3c(N(c4ccc5c(c4)c4ccccc4n5-c4ccccc4)c4ccccc4-c4ccccc4)cc4ccccc4c3o2)cc1 ; c1ccc(-c2nc3c(o2)c(N(c2ccc4c(c2)c2ccccc2n4-c2ccccc2)c2ccccc2-c2ccccc2)cc2ccccc23)cc1 ; c1ccc(-c2nc3c(N(c4ccccc4)c4cccc5c4c4ccccc4n5-c4ccccc4)cc4ccccc4c3o2)cc1 ; c1ccc(-c2nc3c(o2)c(N(c2ccccc2)c2cccc4c2c2ccccc2n4-c2ccccc2)cc2ccccc23)cc1]

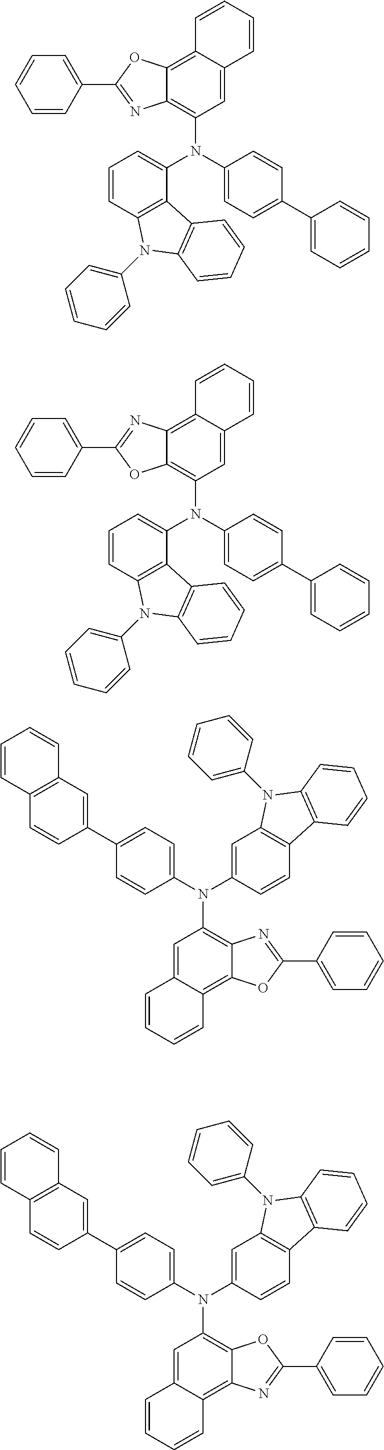 [SMILES: c1ccc(-c2ccc(N(c3cc4ccccc4c4oc(-c5ccccc5)nc34)c3cccc4c3c3ccccc3n4-c3ccccc3)cc2)cc1 ; c1ccc(-c2ccc(N(c3cc4ccccc4c4nc(-c5ccccc5)oc34)c3cccc4c3c3ccccc3n4-c3ccccc3)cc2)cc1 ; c1ccc(-c2nc3c(N(c4ccc(-c5ccc6ccccc6c5)cc4)c4ccc5c6ccccc6n(-c6ccccc6)c5c4)cc4ccccc4c3o2)cc1 ; c1ccc(-c2nc3c(o2)c(N(c2ccc(-c4ccc5ccccc5c4)cc2)c2ccc4c5ccccc5n(-c5ccccc5)c4c2)cc2ccccc23)cc1]

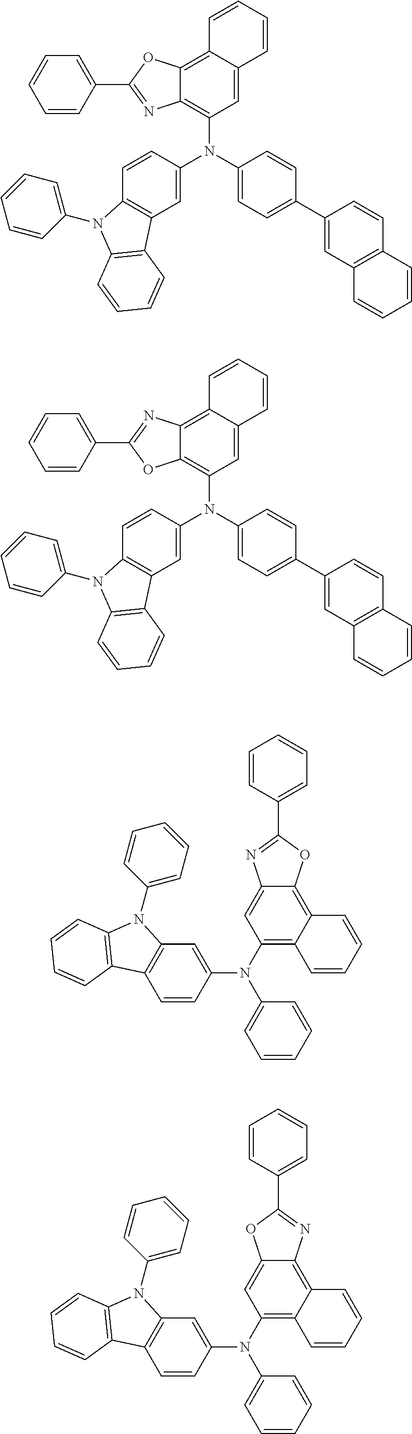 [SMILES: c1ccc(-c2nc3c(N(c4ccc(-c5ccc6ccccc6c5)cc4)c4ccc5c(c4)c4ccccc4n5-c4ccccc4)cc4ccccc4c3o2)cc1 ; c1ccc(-c2nc3c(o2)c(N(c2ccc(-c4ccc5ccccc5c4)cc2)c2ccc4c(c2)c2ccccc2n4-c2ccccc2)cc2ccccc23)cc1 ; c1ccc(-c2nc3cc(N(c4ccccc4)c4ccc5c6ccccc6n(-c6ccccc6)c5c4)c4ccccc4c3o2)cc1 ; c1ccc(-c2nc3c(cc(N(c4ccccc4)c4ccc5c6ccccc6n(-c6ccccc6)c5c4)c4ccccc43)o2)cc1]

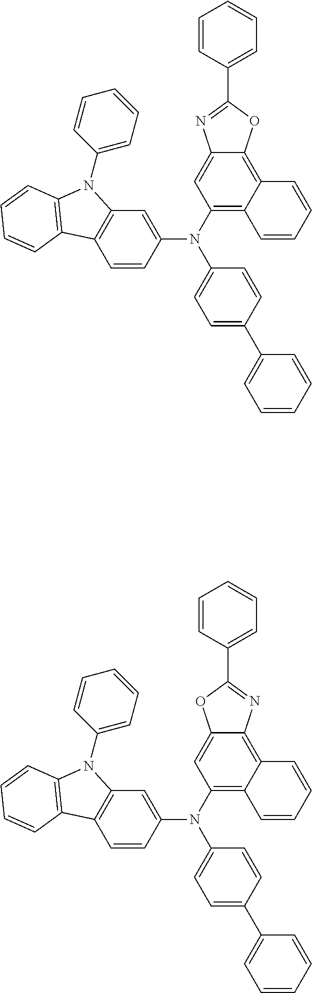 [SMILES: c1ccc(-c2ccc(N(c3ccc4c5ccccc5n(-c5ccccc5)c4c3)c3cc4nc(-c5ccccc5)oc4c4ccccc34)cc2)cc1 ; c1ccc(-c2ccc(N(c3ccc4c5ccccc5n(-c5ccccc5)c4c3)c3cc4oc(-c5ccccc5)nc4c4ccccc34)cc2)cc1]

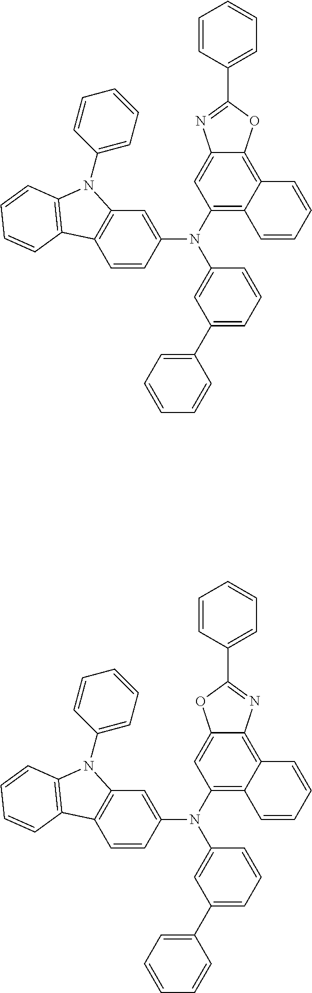 [SMILES: c1ccc(-c2cccc(N(c3ccc4c5ccccc5n(-c5ccccc5)c4c3)c3cc4nc(-c5ccccc5)oc4c4ccccc34)c2)cc1 ; c1ccc(-c2cccc(N(c3ccc4c5ccccc5n(-c5ccccc5)c4c3)c3cc4oc(-c5ccccc5)nc4c4ccccc34)c2)cc1]

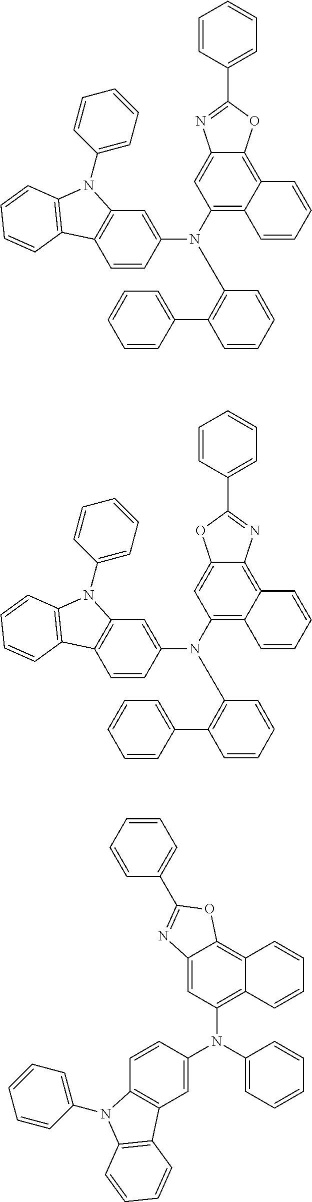 [SMILES: c1ccc(-c2nc3cc(N(c4ccc5c6ccccc6n(-c6ccccc6)c5c4)c4ccccc4-c4ccccc4)c4ccccc4c3o2)cc1 ; c1ccc(-c2nc3c(cc(N(c4ccc5c6ccccc6n(-c6ccccc6)c5c4)c4ccccc4-c4ccccc4)c4ccccc43)o2)cc1 ; c1ccc(-c2nc3cc(N(c4ccccc4)c4ccc5c(c4)c4ccccc4n5-c4ccccc4)c4ccccc4c3o2)cc1]

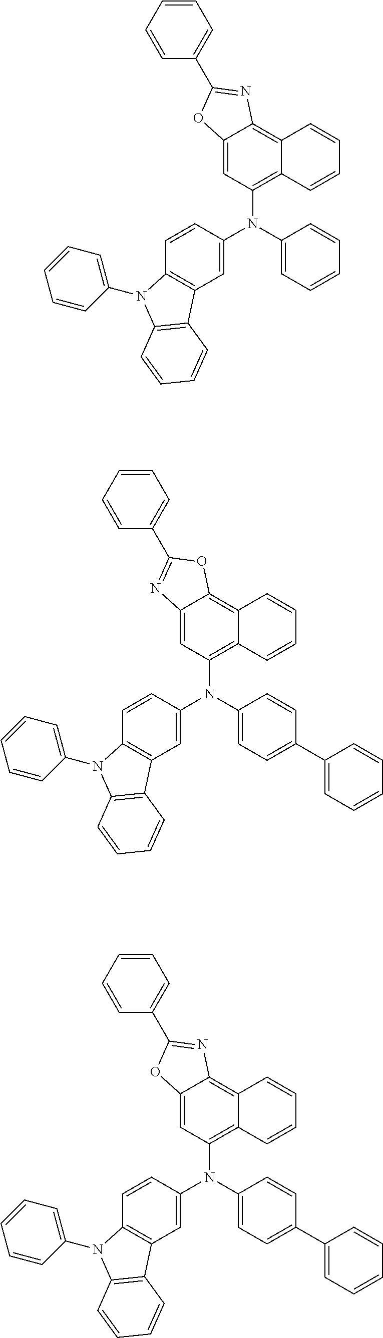 [SMILES: c1ccc(-c2nc3c(cc(N(c4ccccc4)c4ccc5c(c4)c4ccccc4n5-c4ccccc4)c4ccccc43)o2)cc1 ; c1ccc(-c2ccc(N(c3ccc4c(c3)c3ccccc3n4-c3ccccc3)c3cc4nc(-c5ccccc5)oc4c4ccccc34)cc2)cc1 ; c1ccc(-c2ccc(N(c3ccc4c(c3)c3ccccc3n4-c3ccccc3)c3cc4oc(-c5ccccc5)nc4c4ccccc34)cc2)cc1]

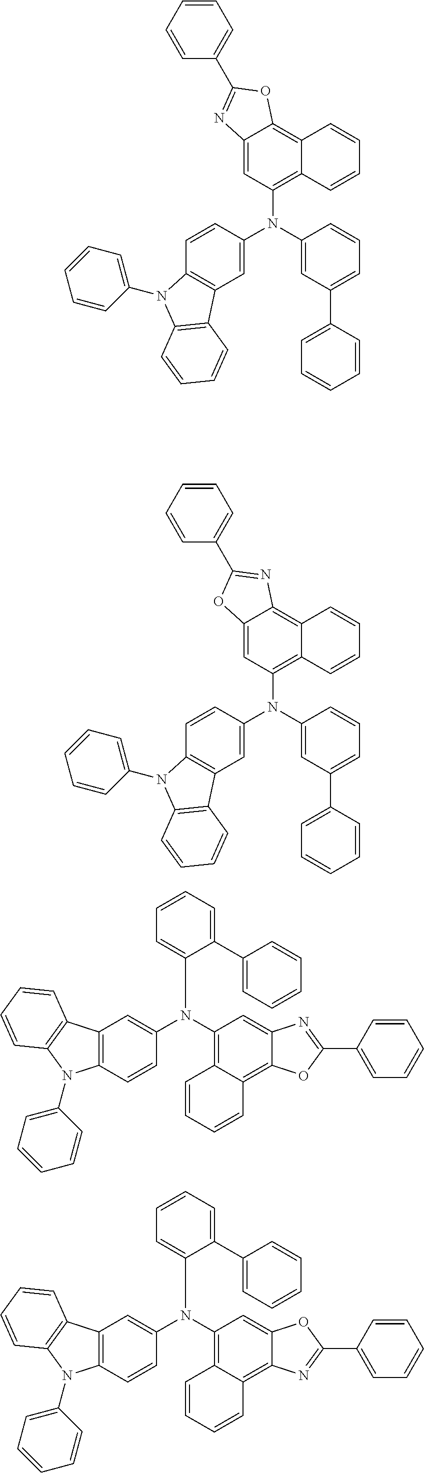

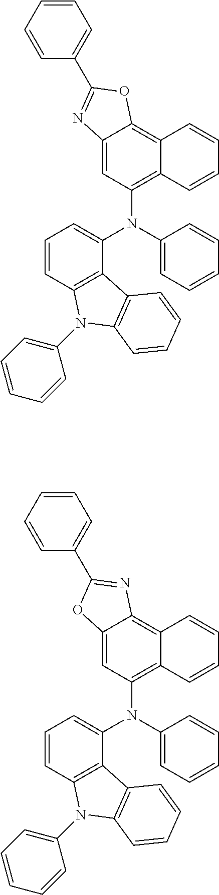 [SMILES: c1ccc(-c2nc3cc(N(c4ccccc4)c4cccc5c4c4ccccc4n5-c4ccccc4)c4ccccc4c3o2)cc1 ; c1ccc(-c2nc3c(cc(N(c4ccccc4)c4cccc5c4c4ccccc4n5-c4ccccc4)c4ccccc43)o2)cc1]

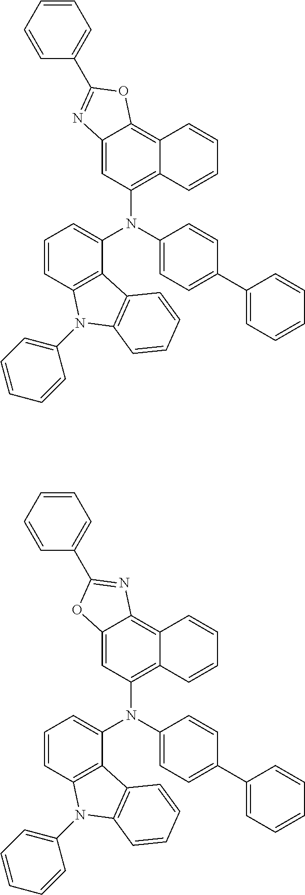 [SMILES: c1ccc(-c2ccc(N(c3cc4nc(-c5ccccc5)oc4c4ccccc34)c3cccc4c3c3ccccc3n4-c3ccccc3)cc2)cc1 ; c1ccc(-c2ccc(N(c3cc4oc(-c5ccccc5)nc4c4ccccc34)c3cccc4c3c3ccccc3n4-c3ccccc3)cc2)cc1]

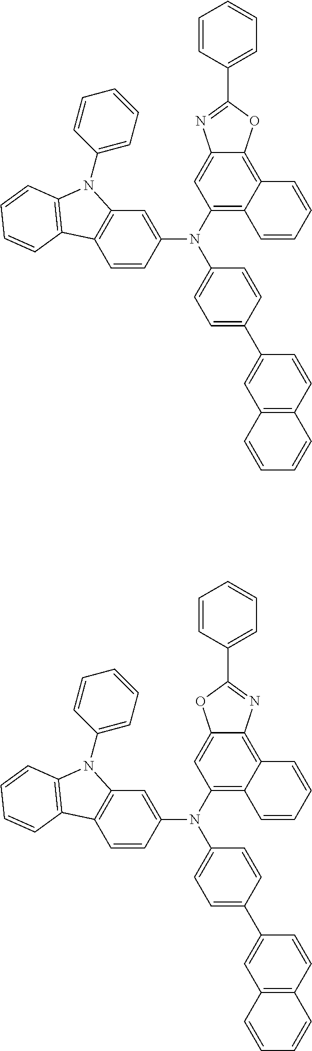 [SMILES: c1ccc(-c2nc3cc(N(c4ccc(-c5ccc6ccccc6c5)cc4)c4ccc5c6ccccc6n(-c6ccccc6)c5c4)c4ccccc4c3o2)cc1 ; c1ccc(-c2nc3c(cc(N(c4ccc(-c5ccc6ccccc6c5)cc4)c4ccc5c6ccccc6n(-c6ccccc6)c5c4)c4ccccc43)o2)cc1]

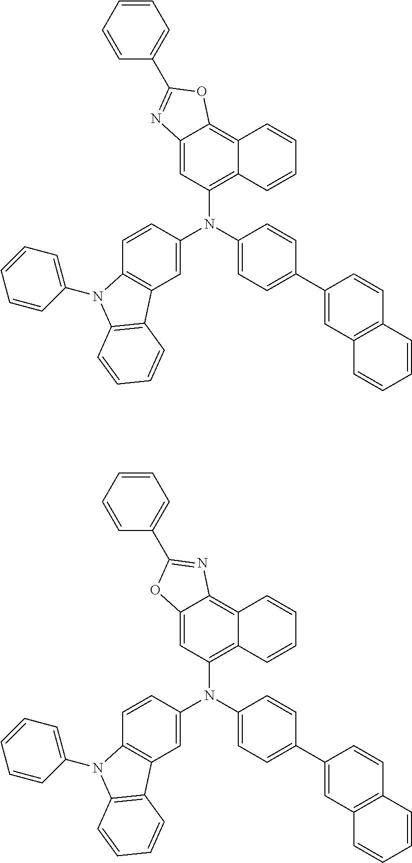 [SMILES: c1ccc(-c2nc3cc(N(c4ccc(-c5ccc6ccccc6c5)cc4)c4ccc5c(c4)c4ccccc4n5-c4ccccc4)c4ccccc4c3o2)cc1 ; c1ccc(-c2nc3c(cc(N(c4ccc(-c5ccc6ccccc6c5)cc4)c4ccc5c(c4)c4ccccc4n5-c4ccccc4)c4ccccc43)o2)cc1]

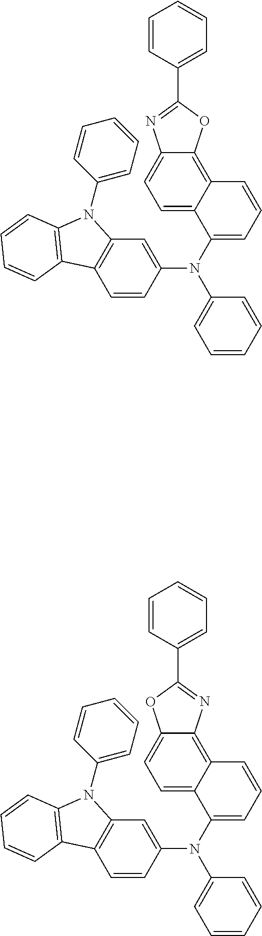 [SMILES: c1ccc(-c2nc3ccc4c(N(c5ccccc5)c5ccc6c7ccccc7n(-c7ccccc7)c6c5)cccc4c3o2)cc1 ; c1ccc(-c2nc3c(ccc4c(N(c5ccccc5)c5ccc6c7ccccc7n(-c7ccccc7)c6c5)cccc43)o2)cc1]

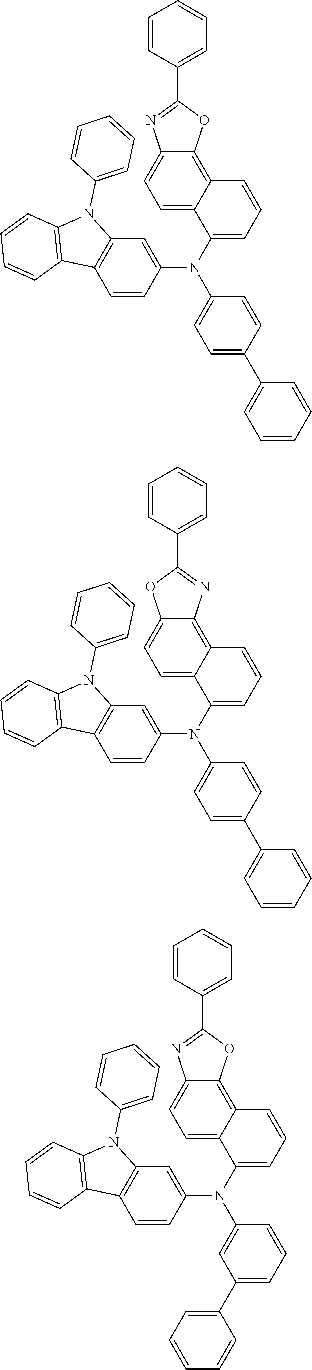 [SMILES: c1ccc(-c2ccc(N(c3ccc4c5ccccc5n(-c5ccccc5)c4c3)c3cccc4c3ccc3nc(-c5ccccc5)oc34)cc2)cc1 ; c1ccc(-c2ccc(N(c3ccc4c5ccccc5n(-c5ccccc5)c4c3)c3cccc4c3ccc3oc(-c5ccccc5)nc34)cc2)cc1 ; c1ccc(-c2cccc(N(c3ccc4c5ccccc5n(-c5ccccc5)c4c3)c3cccc4c3ccc3nc(-c5ccccc5)oc34)c2)cc1]

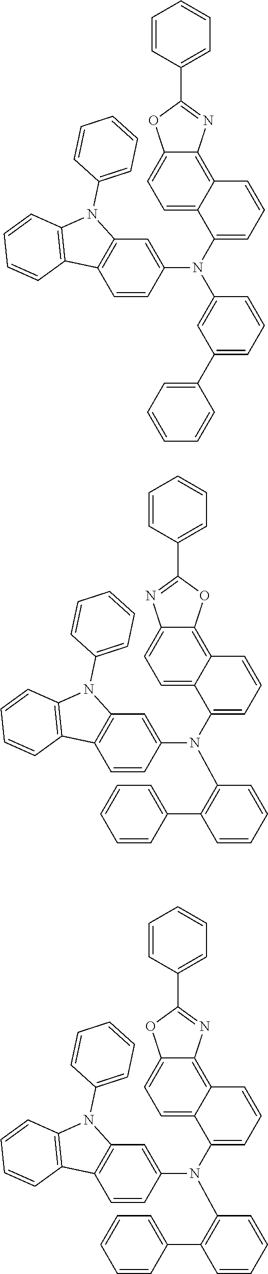

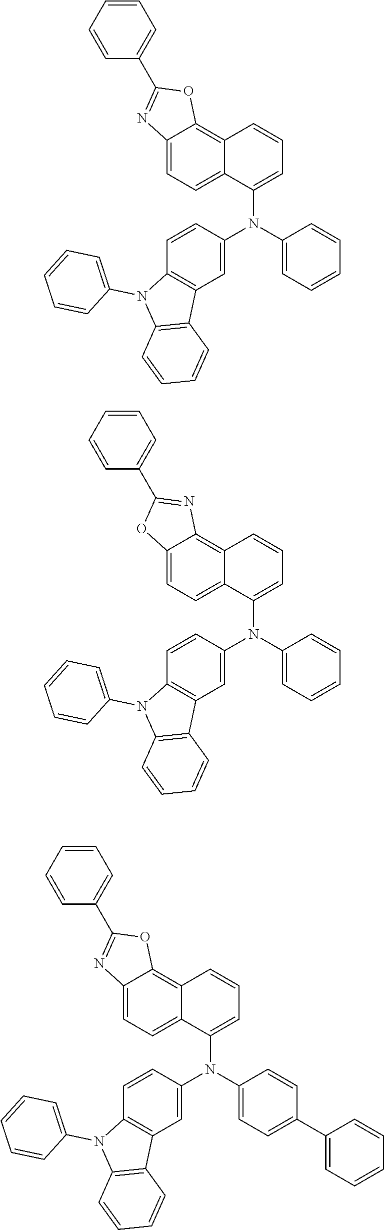

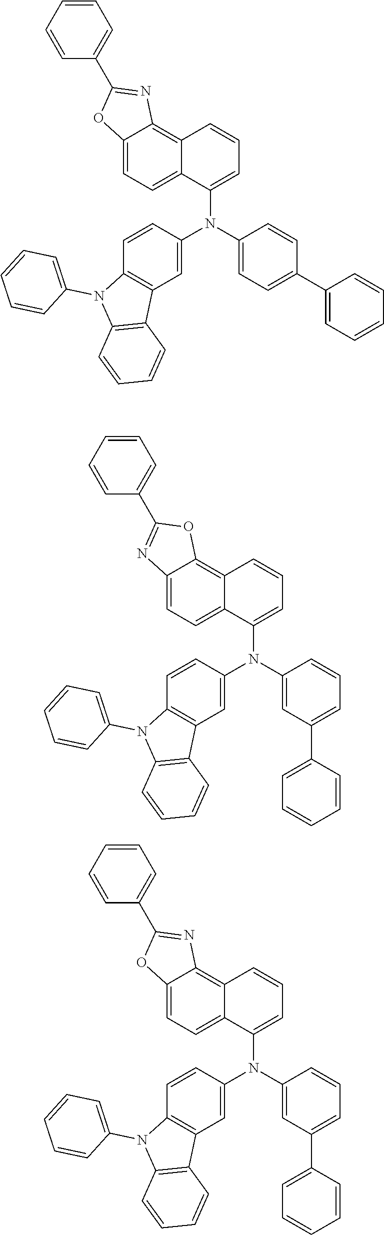

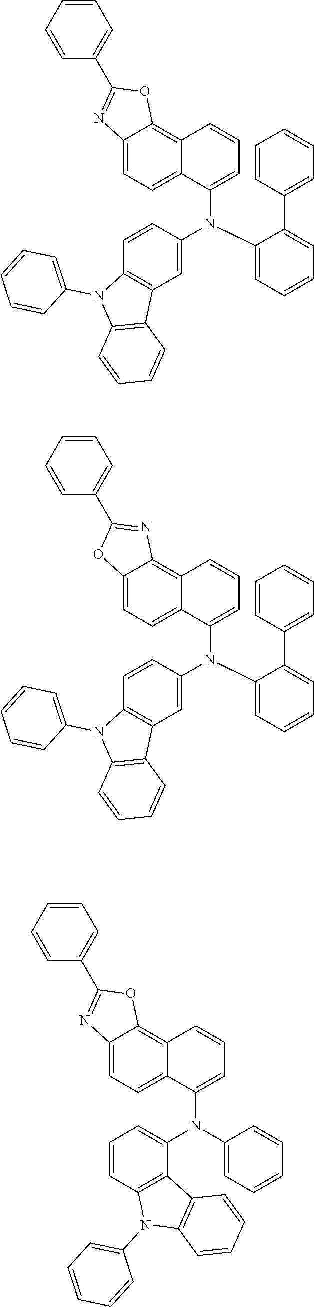 [SMILES: c1ccc(-c2nc3ccc4c(N(c5ccc6c(c5)c5ccccc5n6-c5ccccc5)c5ccccc5-c5ccccc5)cccc4c3o2)cc1 ; c1ccc(-c2nc3c(ccc4c(N(c5ccc6c(c5)c5ccccc5n6-c5ccccc5)c5ccccc5-c5ccccc5)cccc43)o2)cc1 ; c1ccc(-c2nc3ccc4c(N(c5ccccc5)c5cccc6c5c5ccccc5n6-c5ccccc5)cccc4c3o2)cc1]

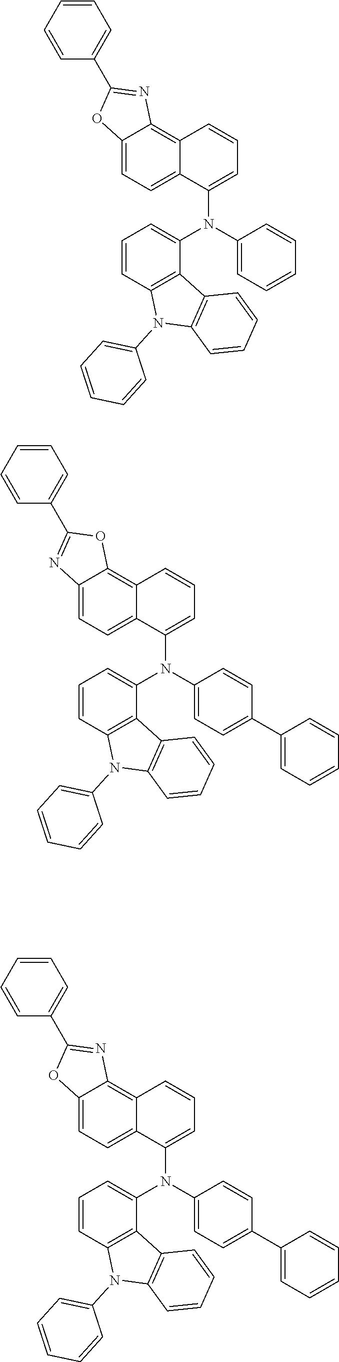 [SMILES: c1ccc(-c2nc3c(ccc4c(N(c5ccccc5)c5cccc6c5c5ccccc5n6-c5ccccc5)cccc43)o2)cc1 ; c1ccc(-c2ccc(N(c3cccc4c3ccc3nc(-c5ccccc5)oc34)c3cccc4c3c3ccccc3n4-c3ccccc3)cc2)cc1 ; c1ccc(-c2ccc(N(c3cccc4c3ccc3oc(-c5ccccc5)nc34)c3cccc4c3c3ccccc3n4-c3ccccc3)cc2)cc1]

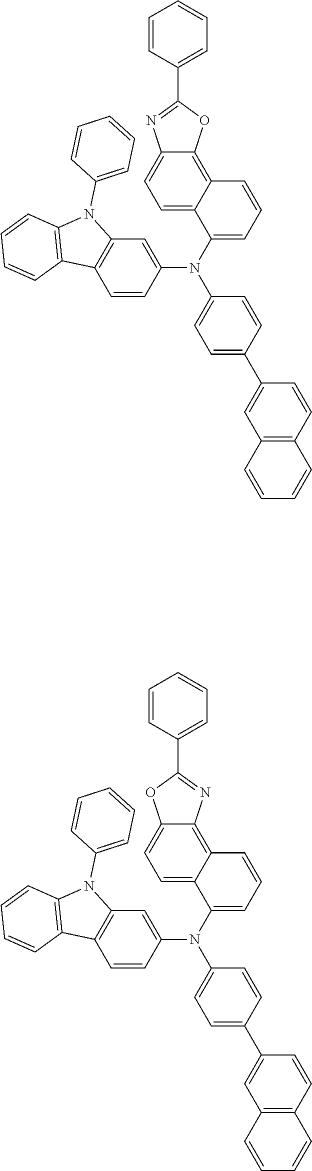 [SMILES: c1ccc(-c2nc3ccc4c(N(c5ccc(-c6ccc7ccccc7c6)cc5)c5ccc6c7ccccc7n(-c7ccccc7)c6c5)cccc4c3o2)cc1 ; c1ccc(-c2nc3c(ccc4c(N(c5ccc(-c6ccc7ccccc7c6)cc5)c5ccc6c7ccccc7n(-c7ccccc7)c6c5)cccc43)o2)cc1]

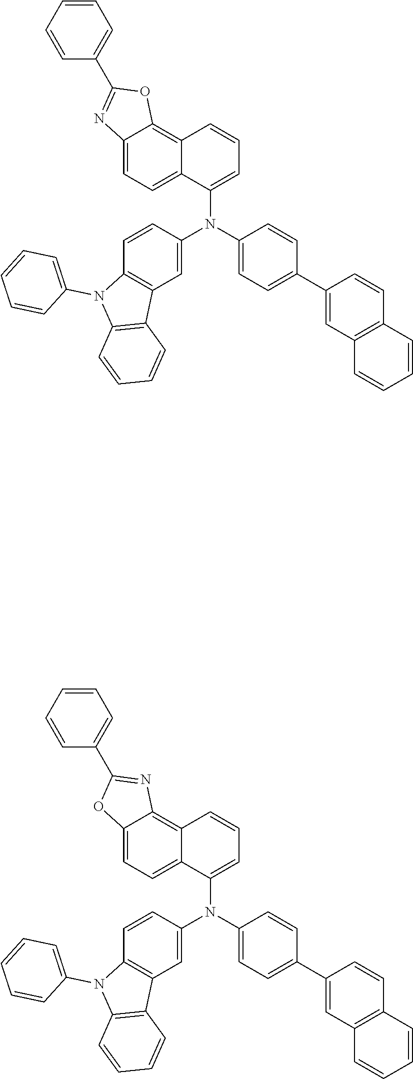 [SMILES: c1ccc(-c2nc3ccc4c(N(c5ccc(-c6ccc7ccccc7c6)cc5)c5ccc6c(c5)c5ccccc5n6-c5ccccc5)cccc4c3o2)cc1 ; c1ccc(-c2nc3c(ccc4c(N(c5ccc(-c6ccc7ccccc7c6)cc5)c5ccc6c(c5)c5ccccc5n6-c5ccccc5)cccc43)o2)cc1]

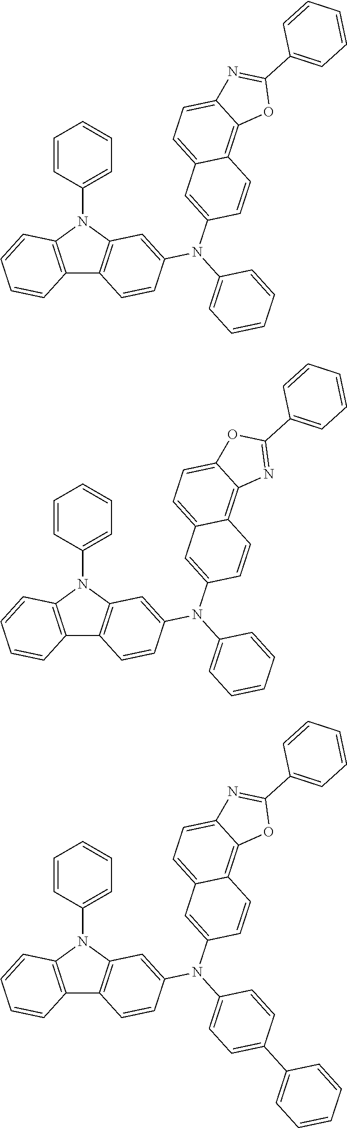 [SMILES: c1ccc(-c2nc3ccc4cc(N(c5ccccc5)c5ccc6c7ccccc7n(-c7ccccc7)c6c5)ccc4c3o2)cc1 ; c1ccc(-c2nc3c(ccc4cc(N(c5ccccc5)c5ccc6c7ccccc7n(-c7ccccc7)c6c5)ccc43)o2)cc1 ; c1ccc(-c2ccc(N(c3ccc4c(ccc5nc(-c6ccccc6)oc54)c3)c3ccc4c5ccccc5n(-c5ccccc5)c4c3)cc2)cc1]

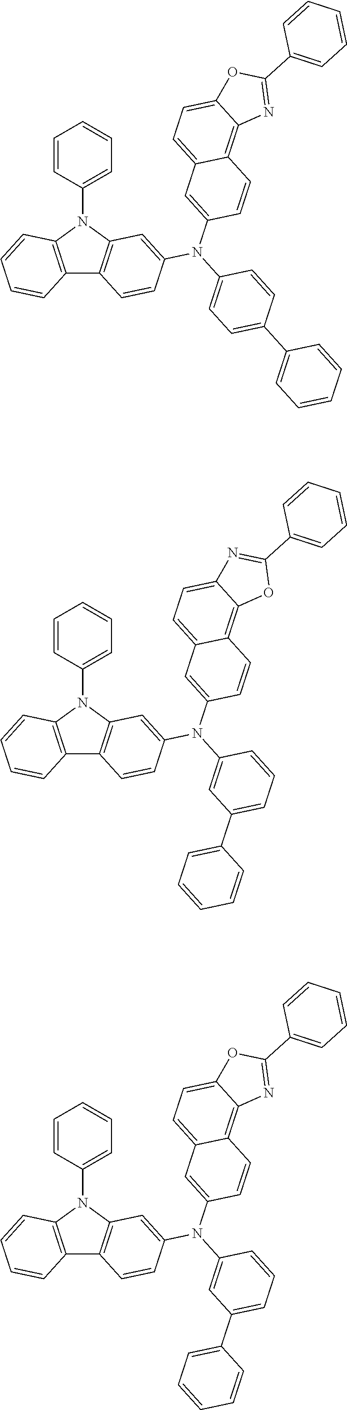 [SMILES: c1ccc(-c2ccc(N(c3ccc4c(ccc5oc(-c6ccccc6)nc54)c3)c3ccc4c5ccccc5n(-c5ccccc5)c4c3)cc2)cc1 ; c1ccc(-c2cccc(N(c3ccc4c(ccc5nc(-c6ccccc6)oc54)c3)c3ccc4c5ccccc5n(-c5ccccc5)c4c3)c2)cc1 ; c1ccc(-c2cccc(N(c3ccc4c(ccc5oc(-c6ccccc6)nc54)c3)c3ccc4c5ccccc5n(-c5ccccc5)c4c3)c2)cc1]

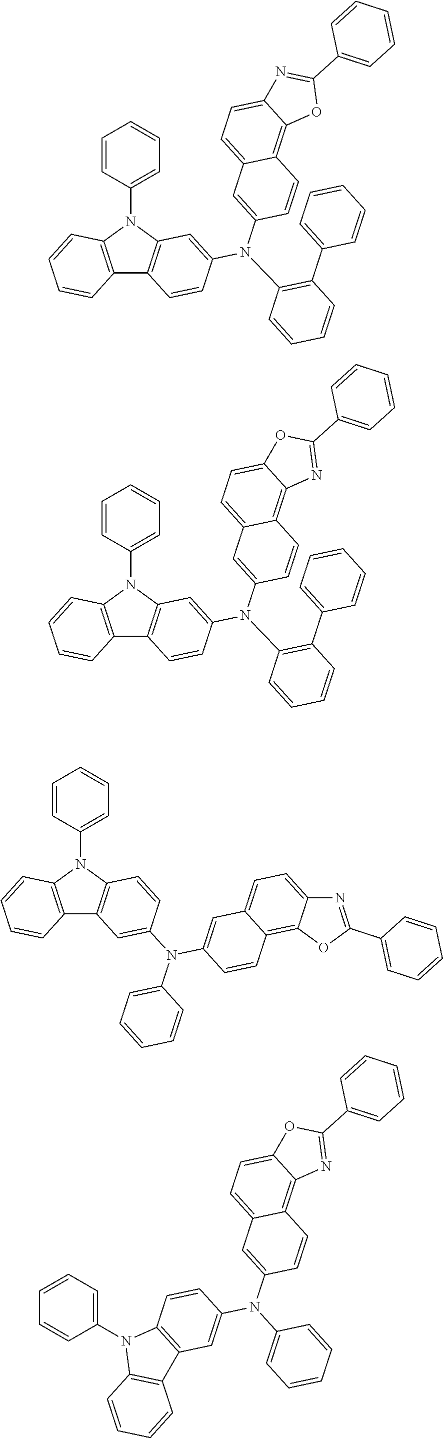

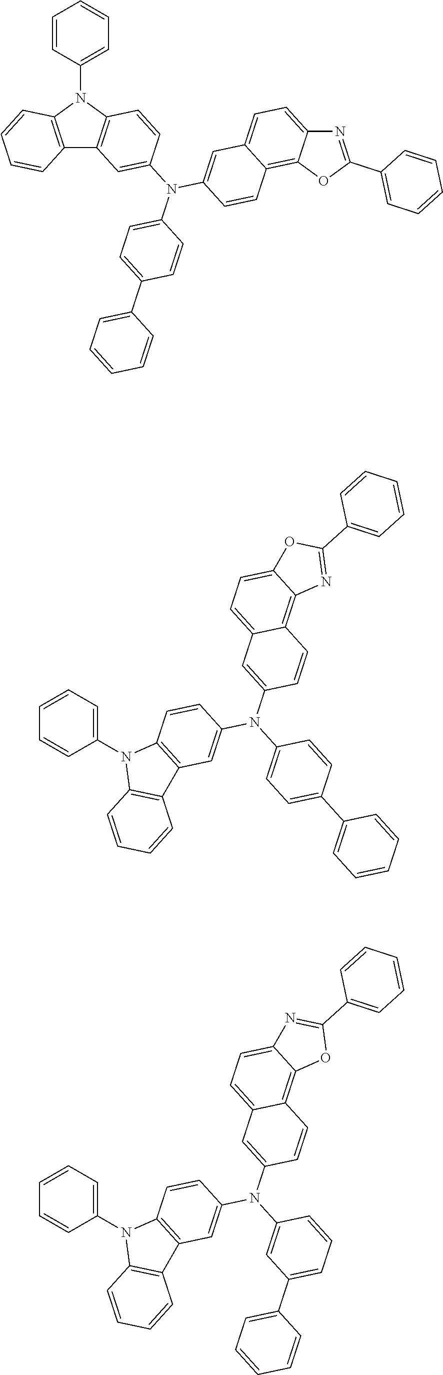 [SMILES: c1ccc(-c2ccc(N(c3ccc4c(ccc5nc(-c6ccccc6)oc54)c3)c3ccc4c(c3)c3ccccc3n4-c3ccccc3)cc2)cc1 ; c1ccc(-c2ccc(N(c3ccc4c(ccc5oc(-c6ccccc6)nc54)c3)c3ccc4c(c3)c3ccccc3n4-c3ccccc3)cc2)cc1 ; c1ccc(-c2cccc(N(c3ccc4c(ccc5nc(-c6ccccc6)oc54)c3)c3ccc4c(c3)c3ccccc3n4-c3ccccc3)c2)cc1]

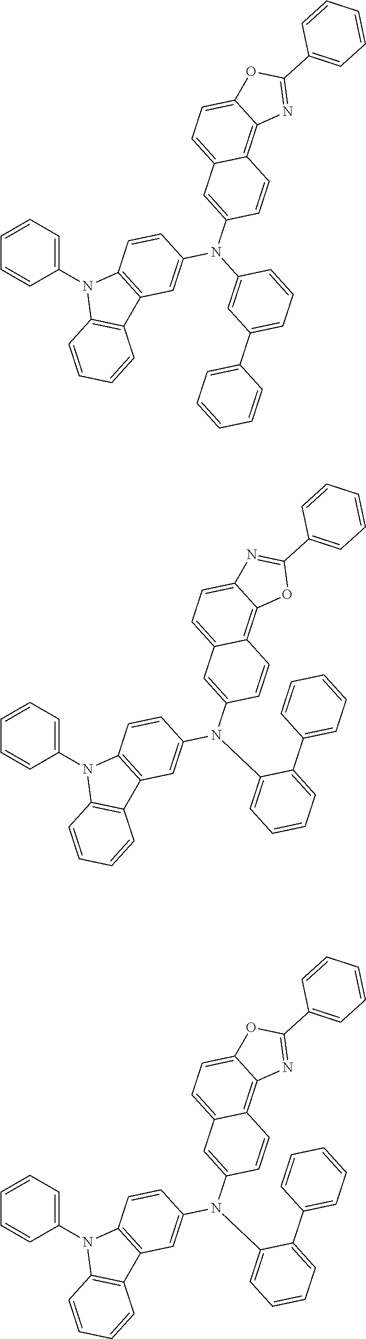 [SMILES: c1ccc(-c2cccc(N(c3ccc4c(ccc5oc(-c6ccccc6)nc54)c3)c3ccc4c(c3)c3ccccc3n4-c3ccccc3)c2)cc1 ; c1ccc(-c2nc3ccc4cc(N(c5ccc6c(c5)c5ccccc5n6-c5ccccc5)c5ccccc5-c5ccccc5)ccc4c3o2)cc1 ; c1ccc(-c2nc3c(ccc4cc(N(c5ccc6c(c5)c5ccccc5n6-c5ccccc5)c5ccccc5-c5ccccc5)ccc43)o2)cc1]

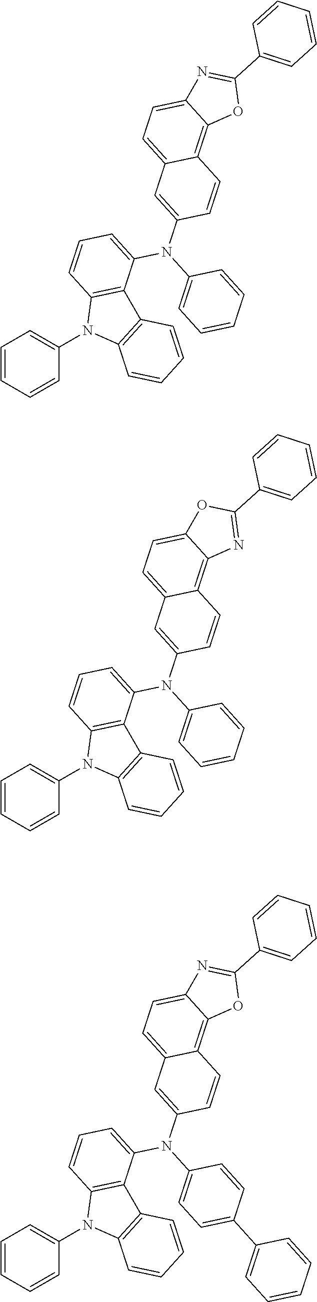 [SMILES: c1ccc(-c2nc3ccc4cc(N(c5ccccc5)c5cccc6c5c5ccccc5n6-c5ccccc5)ccc4c3o2)cc1 ; c1ccc(-c2nc3c(ccc4cc(N(c5ccccc5)c5cccc6c5c5ccccc5n6-c5ccccc5)ccc43)o2)cc1 ; c1ccc(-c2ccc(N(c3ccc4c(ccc5nc(-c6ccccc6)oc54)c3)c3cccc4c3c3ccccc3n4-c3ccccc3)cc2)cc1]

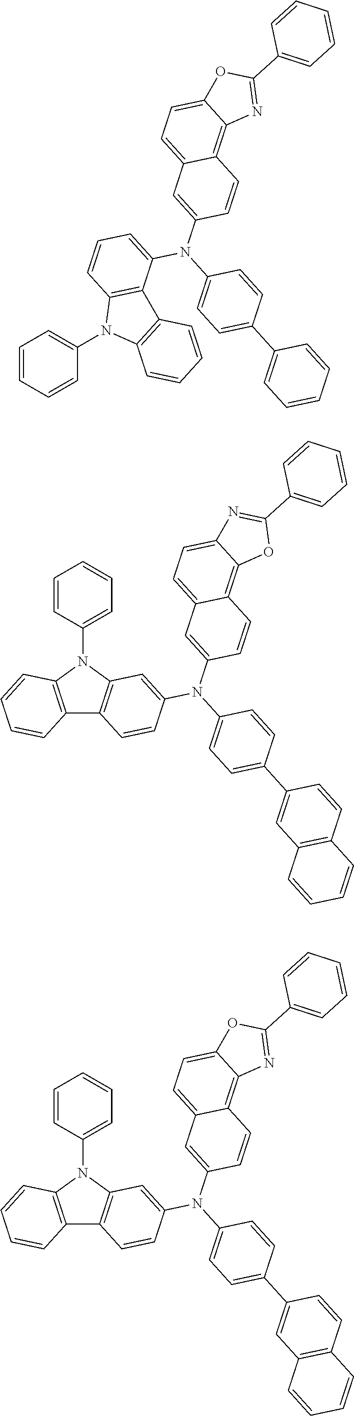

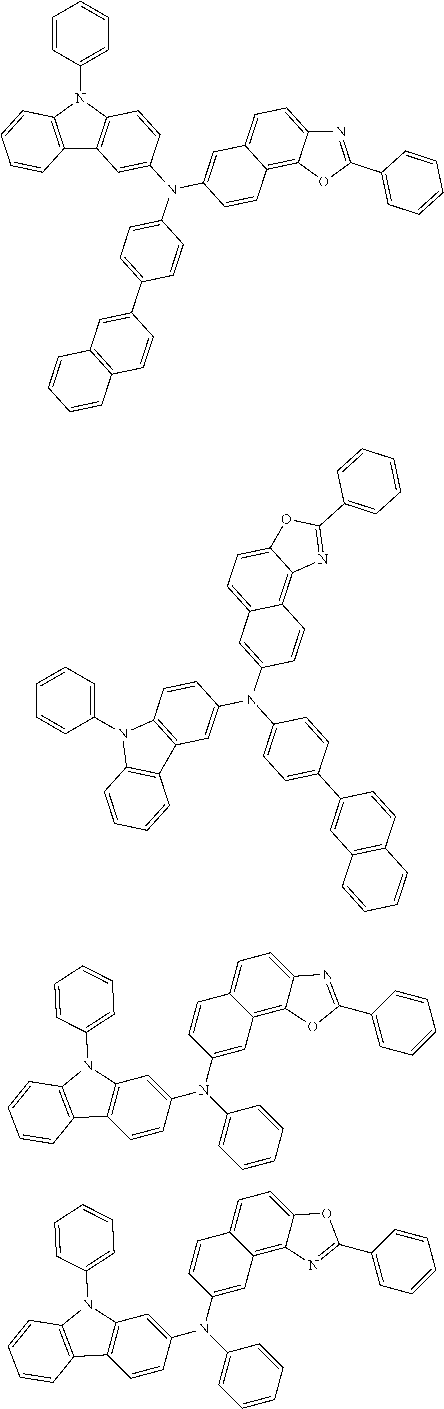

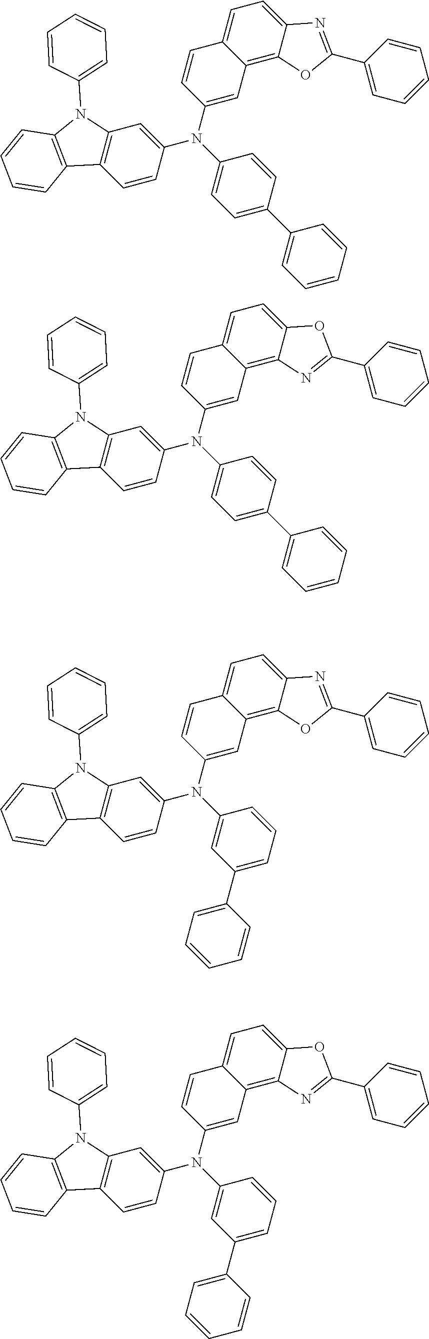

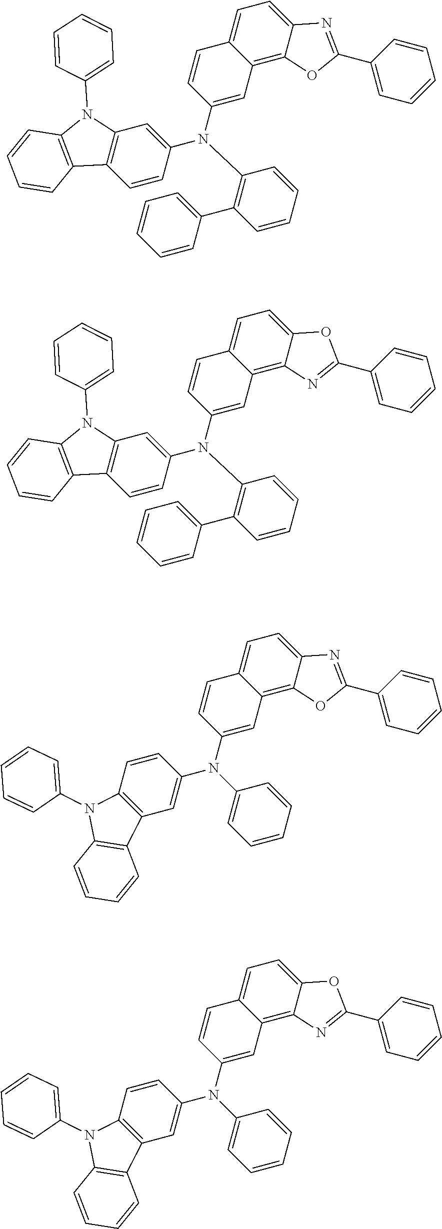 [SMILES: c1ccc(-c2nc3ccc4ccc(N(c5ccc6c7ccccc7n(-c7ccccc7)c6c5)c5ccccc5-c5ccccc5)cc4c3o2)cc1 ; c1ccc(-c2nc3c(ccc4ccc(N(c5ccc6c7ccccc7n(-c7ccccc7)c6c5)c5ccccc5-c5ccccc5)cc43)o2)cc1 ; c1ccc(-c2nc3ccc4ccc(N(c5ccccc5)c5ccc6c(c5)c5ccccc5n6-c5ccccc5)cc4c3o2)cc1 ; c1ccc(-c2nc3c(ccc4ccc(N(c5ccccc5)c5ccc6c(c5)c5ccccc5n6-c5ccccc5)cc43)o2)cc1]

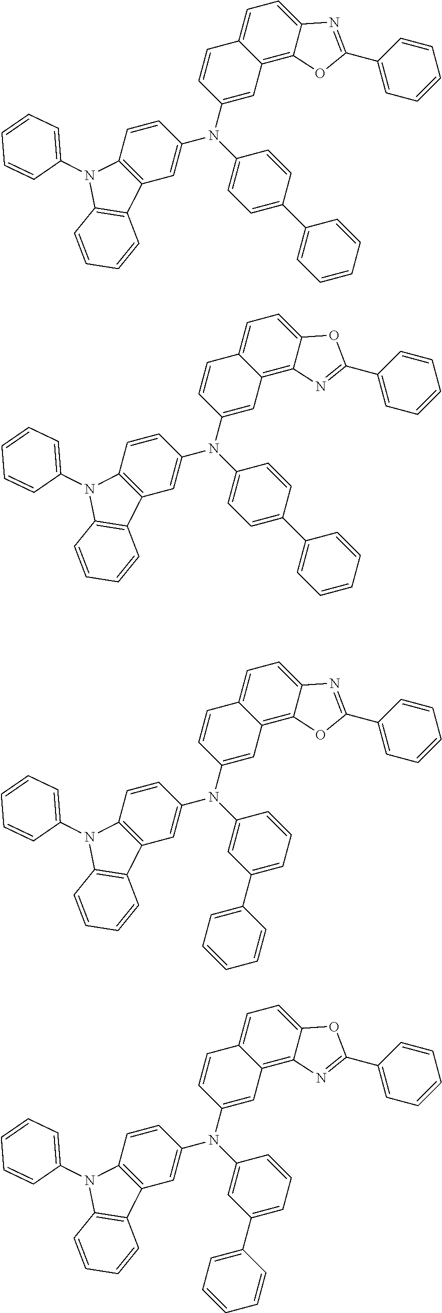 [SMILES: c1ccc(-c2ccc(N(c3ccc4ccc5nc(-c6ccccc6)oc5c4c3)c3ccc4c(c3)c3ccccc3n4-c3ccccc3)cc2)cc1 ; c1ccc(-c2ccc(N(c3ccc4ccc5oc(-c6ccccc6)nc5c4c3)c3ccc4c(c3)c3ccccc3n4-c3ccccc3)cc2)cc1 ; c1ccc(-c2cccc(N(c3ccc4ccc5nc(-c6ccccc6)oc5c4c3)c3ccc4c(c3)c3ccccc3n4-c3ccccc3)c2)cc1 ; c1ccc(-c2cccc(N(c3ccc4ccc5oc(-c6ccccc6)nc5c4c3)c3ccc4c(c3)c3ccccc3n4-c3ccccc3)c2)cc1]

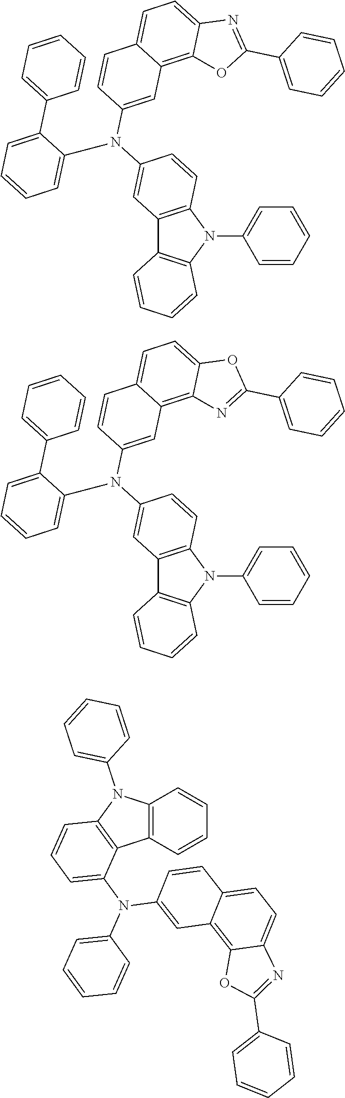

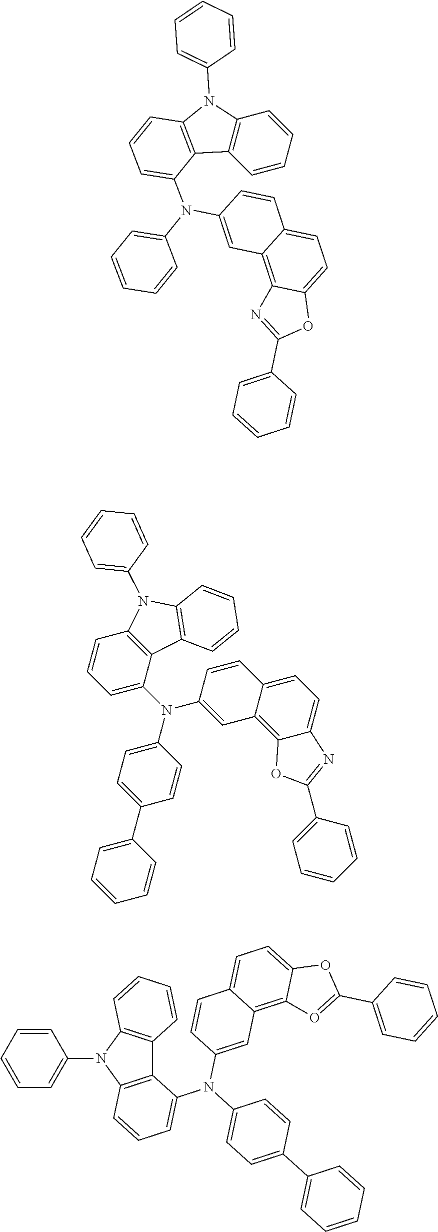

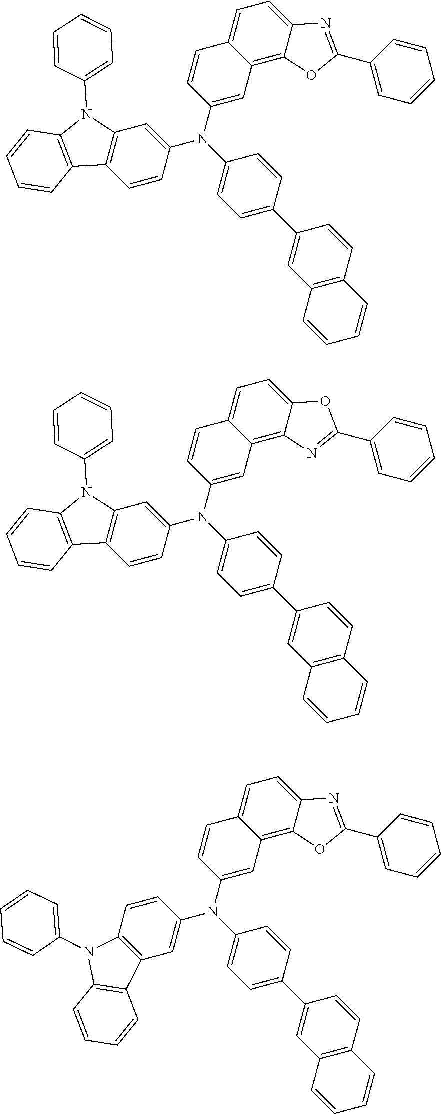 [SMILES: c1ccc(-c2nc3ccc4ccc(N(c5ccc(-c6ccc7ccccc7c6)cc5)c5ccc6c7ccccc7n(-c7ccccc7)c6c5)cc4c3o2)cc1 ; c1ccc(-c2nc3c(ccc4ccc(N(c5ccc(-c6ccc7ccccc7c6)cc5)c5ccc6c7ccccc7n(-c7ccccc7)c6c5)cc43)o2)cc1 ; c1ccc(-c2nc3ccc4ccc(N(c5ccc(-c6ccc7ccccc7c6)cc5)c5ccc6c(c5)c5ccccc5n6-c5ccccc5)cc4c3o2)cc1]

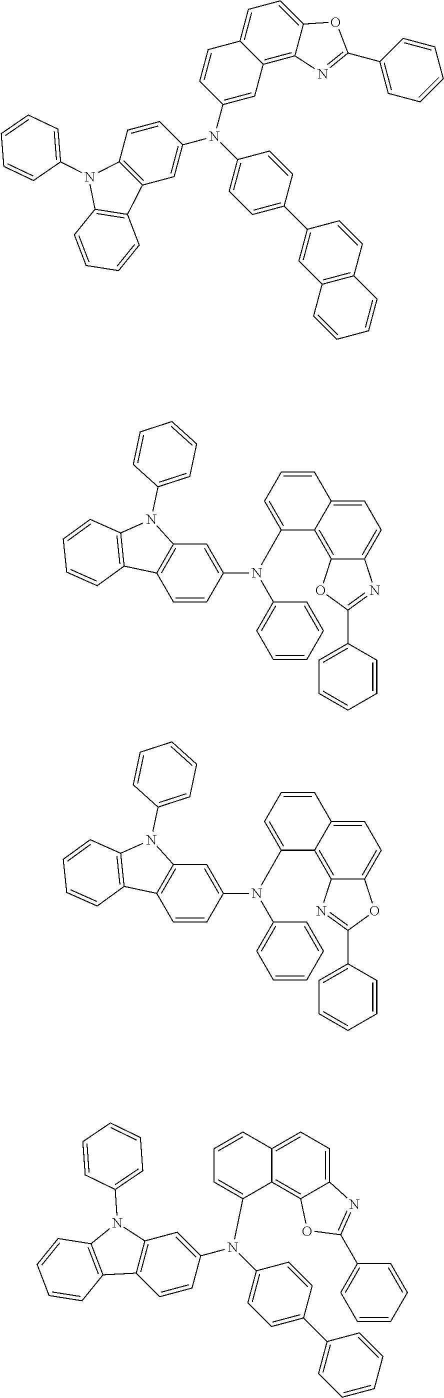 [SMILES: c1ccc(-c2nc3c(ccc4ccc(N(c5ccc(-c6ccc7ccccc7c6)cc5)c5ccc6c(c5)c5ccccc5n6-c5ccccc5)cc43)o2)cc1 ; c1ccc(-c2nc3ccc4cccc(N(c5ccccc5)c5ccc6c7ccccc7n(-c7ccccc7)c6c5)c4c3o2)cc1 ; c1ccc(-c2nc3c(ccc4cccc(N(c5ccccc5)c5ccc6c7ccccc7n(-c7ccccc7)c6c5)c43)o2)cc1 ; c1ccc(-c2ccc(N(c3ccc4c5ccccc5n(-c5ccccc5)c4c3)c3cccc4ccc5nc(-c6ccccc6)oc5c34)cc2)cc1]

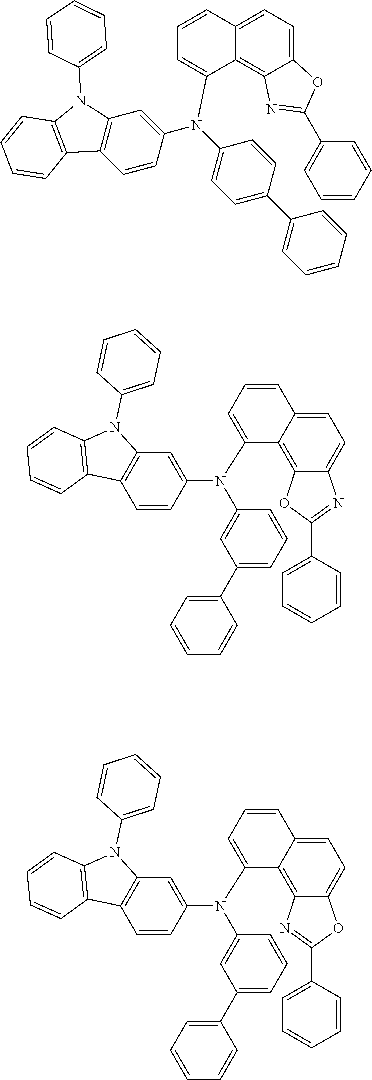 [SMILES: c1ccc(-c2ccc(N(c3ccc4c5ccccc5n(-c5ccccc5)c4c3)c3cccc4ccc5oc(-c6ccccc6)nc5c34)cc2)cc1 ; c1ccc(-c2cccc(N(c3ccc4c5ccccc5n(-c5ccccc5)c4c3)c3cccc4ccc5nc(-c6ccccc6)oc5c34)c2)cc1 ; c1ccc(-c2cccc(N(c3ccc4c5ccccc5n(-c5ccccc5)c4c3)c3cccc4ccc5oc(-c6ccccc6)nc5c34)c2)cc1]

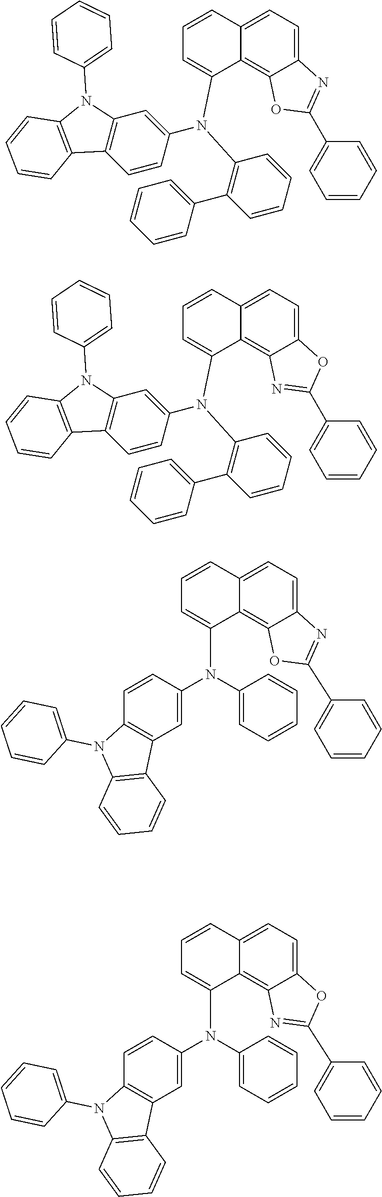 [SMILES: c1ccc(-c2nc3ccc4cccc(N(c5ccc6c7ccccc7n(-c7ccccc7)c6c5)c5ccccc5-c5ccccc5)c4c3o2)cc1 ; c1ccc(-c2nc3c(ccc4cccc(N(c5ccc6c7ccccc7n(-c7ccccc7)c6c5)c5ccccc5-c5ccccc5)c43)o2)cc1 ; c1ccc(-c2nc3ccc4cccc(N(c5ccccc5)c5ccc6c(c5)c5ccccc5n6-c5ccccc5)c4c3o2)cc1 ; c1ccc(-c2nc3c(ccc4cccc(N(c5ccccc5)c5ccc6c(c5)c5ccccc5n6-c5ccccc5)c43)o2)cc1]

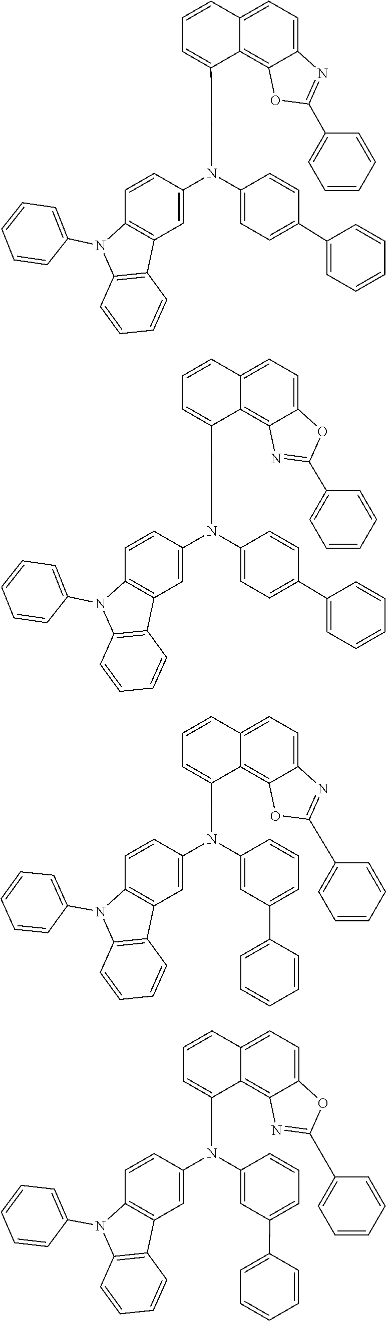

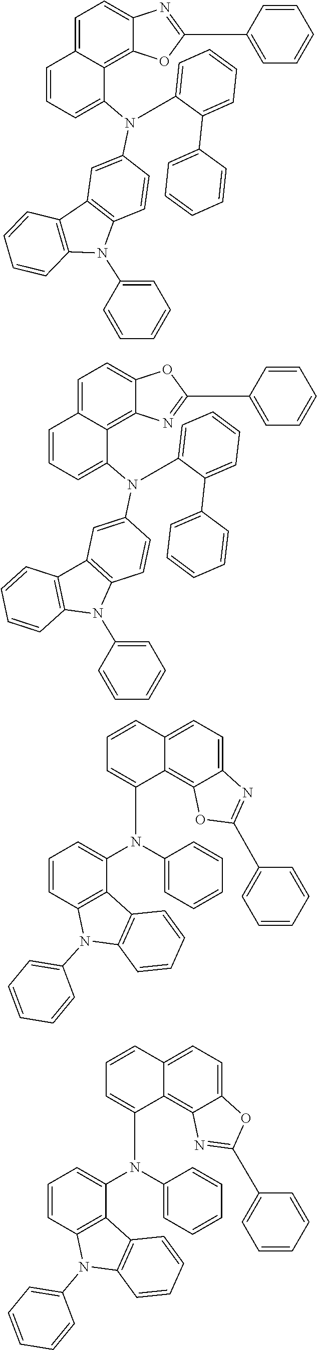

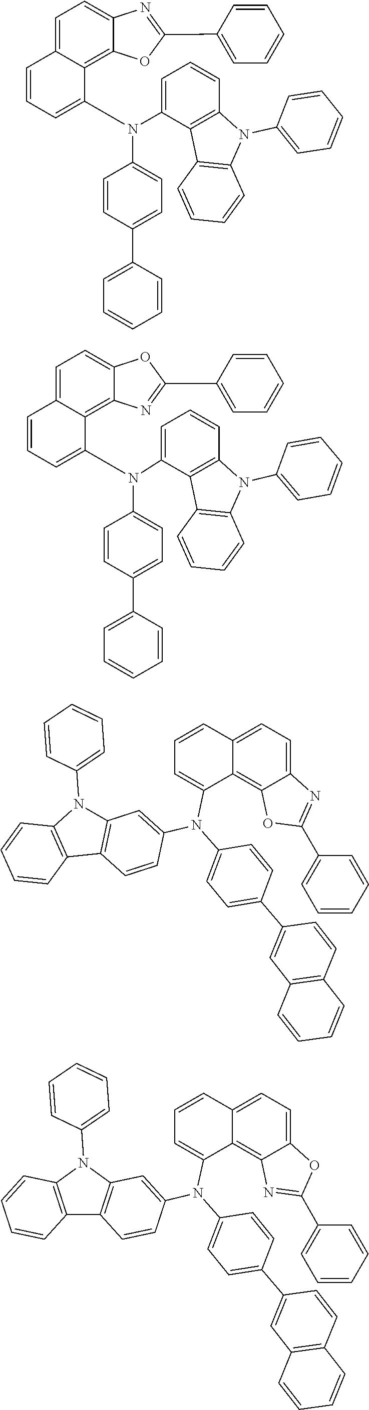

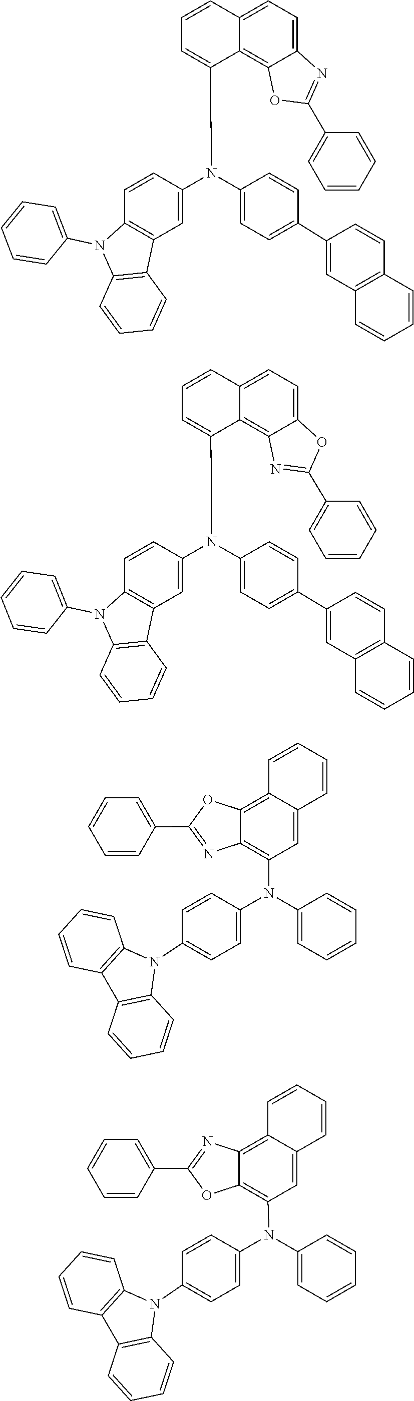 [SMILES: c1ccc(-c2nc3ccc4cccc(N(c5ccc(-c6ccc7ccccc7c6)cc5)c5ccc6c(c5)c5ccccc5n6-c5ccccc5)c4c3o2)cc1 ; c1ccc(-c2nc3c(ccc4cccc(N(c5ccc(-c6ccc7ccccc7c6)cc5)c5ccc6c(c5)c5ccccc5n6-c5ccccc5)c43)o2)cc1 ; c1ccc(-c2nc3c(N(c4ccccc4)c4ccc(-n5c6ccccc6c6ccccc65)cc4)cc4ccccc4c3o2)cc1 ; c1ccc(-c2nc3c(o2)c(N(c2ccccc2)c2ccc(-n4c5ccccc5c5ccccc54)cc2)cc2ccccc23)cc1]

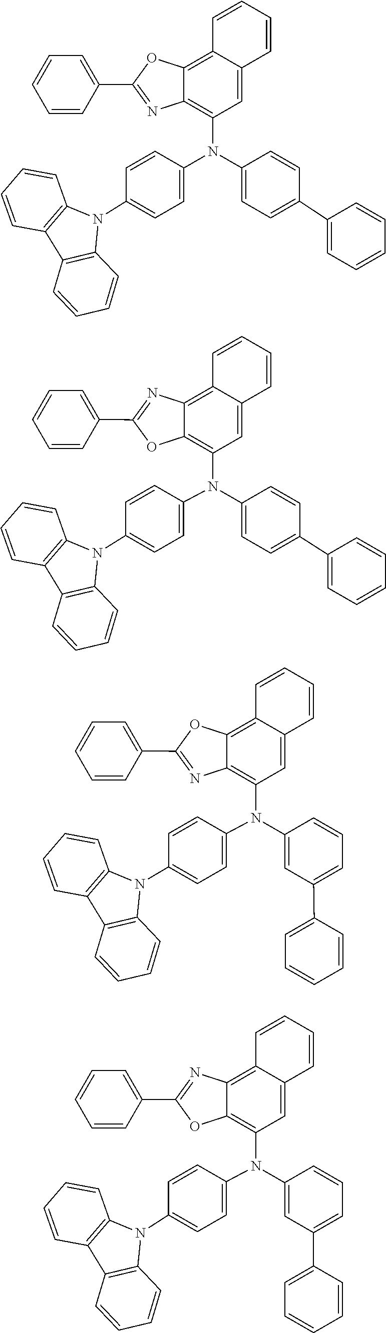

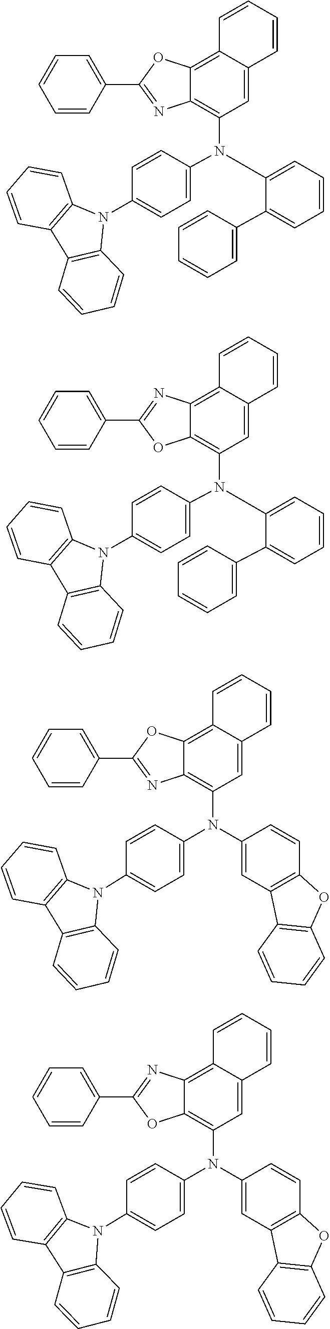

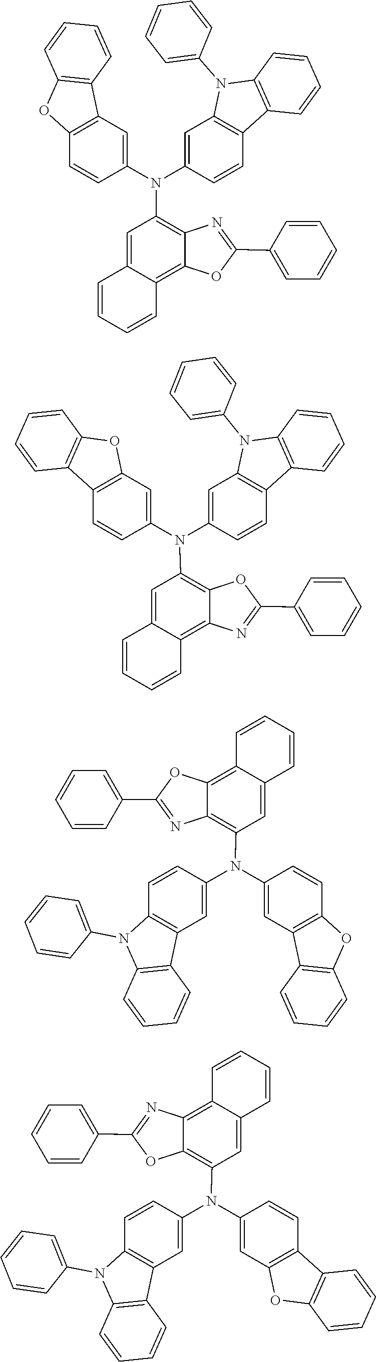 [SMILES: c1ccc(-c2nc3c(N(c4ccc5oc6ccccc6c5c4)c4ccc5c6ccccc6n(-c6ccccc6)c5c4)cc4ccccc4c3o2)cc1 ; c1ccc(-c2nc3c(o2)c(N(c2ccc4c(c2)oc2ccccc24)c2ccc4c5ccccc5n(-c5ccccc5)c4c2)cc2ccccc23)cc1 ; c1ccc(-c2nc3c(N(c4ccc5oc6ccccc6c5c4)c4ccc5c(c4)c4ccccc4n5-c4ccccc4)cc4ccccc4c3o2)cc1 ; c1ccc(-c2nc3c(o2)c(N(c2ccc4c(c2)oc2ccccc24)c2ccc4c(c2)c2ccccc2n4-c2ccccc2)cc2ccccc23)cc1]

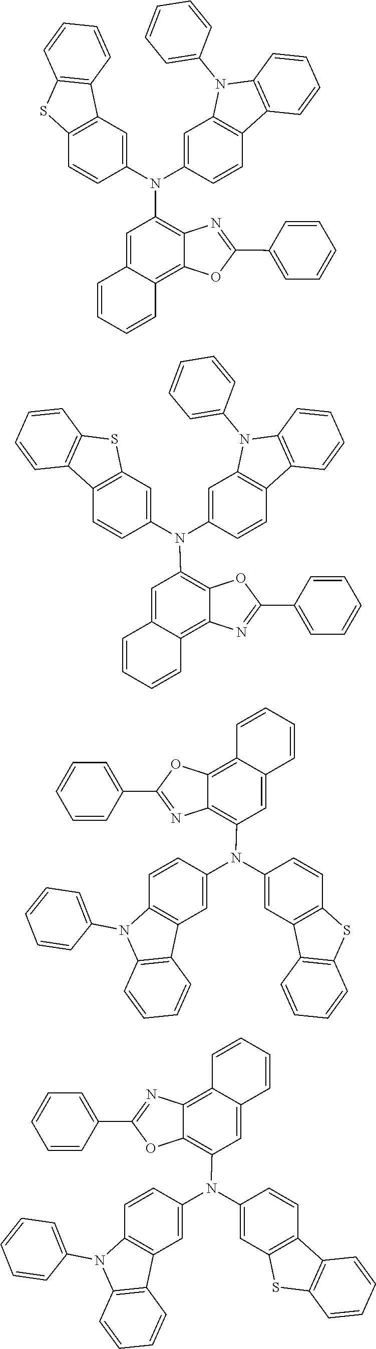 [SMILES: c1ccc(-c2nc3c(N(c4ccc5sc6ccccc6c5c4)c4ccc5c6ccccc6n(-c6ccccc6)c5c4)cc4ccccc4c3o2)cc1 ; c1ccc(-c2nc3c(o2)c(N(c2ccc4c(c2)sc2ccccc24)c2ccc4c5ccccc5n(-c5ccccc5)c4c2)cc2ccccc23)cc1 ; c1ccc(-c2nc3c(N(c4ccc5sc6ccccc6c5c4)c4ccc5c(c4)c4ccccc4n5-c4ccccc4)cc4ccccc4c3o2)cc1 ; c1ccc(-c2nc3c(o2)c(N(c2ccc4c(c2)sc2ccccc24)c2ccc4c(c2)c2ccccc2n4-c2ccccc2)cc2ccccc23)cc1]

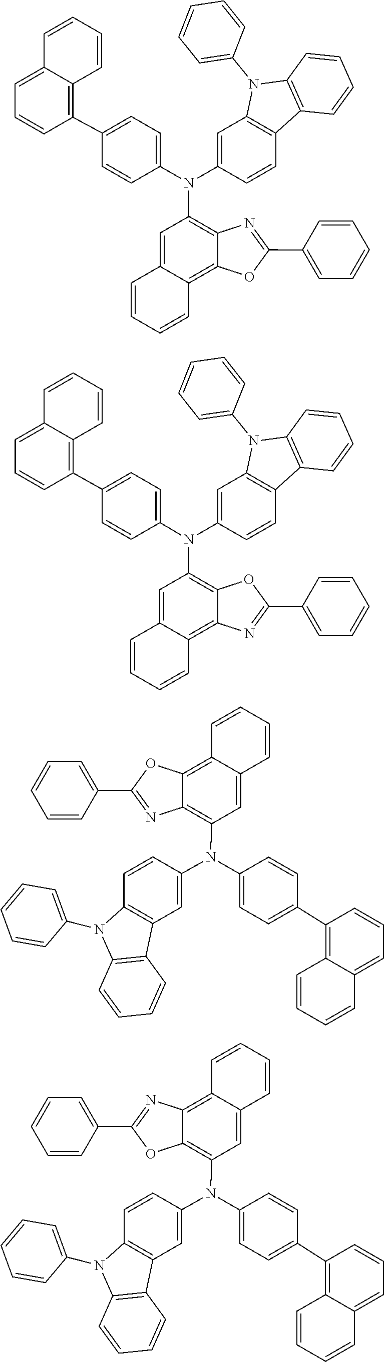

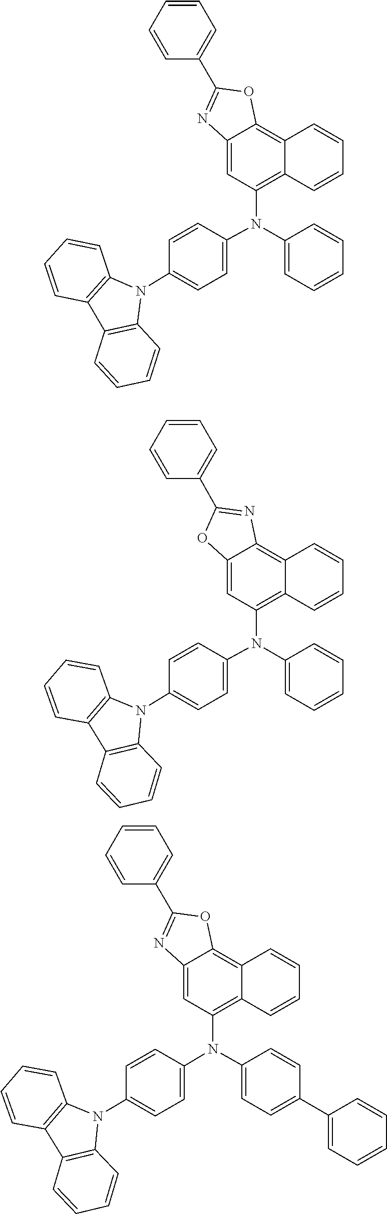

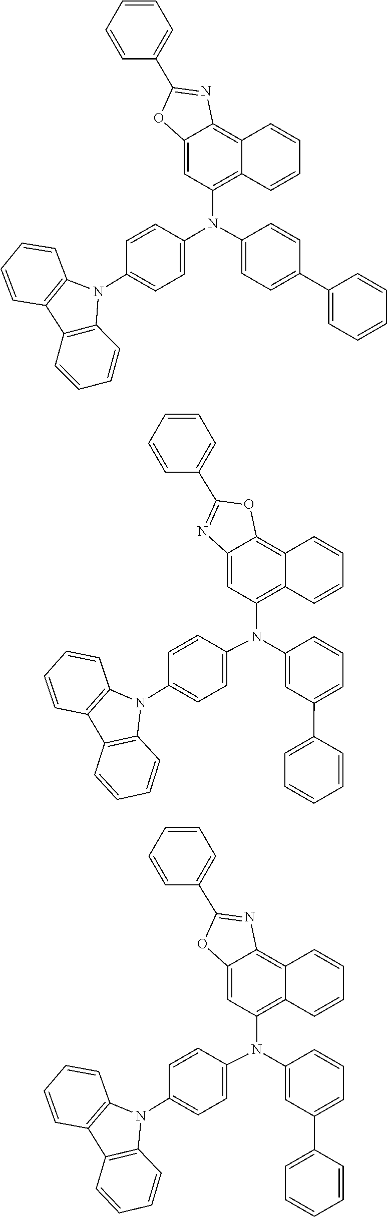

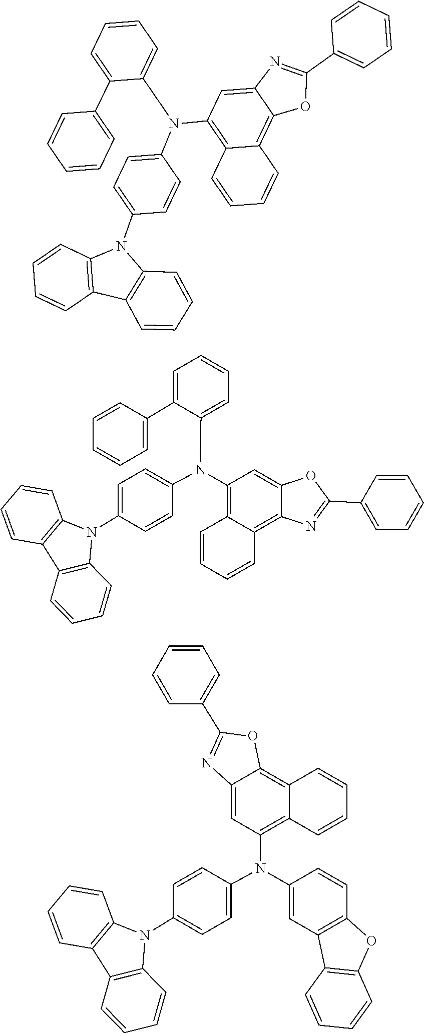

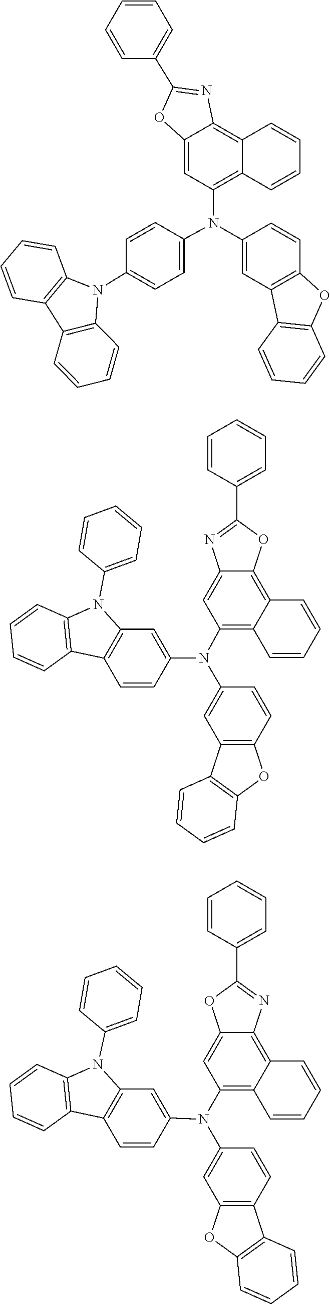 [SMILES: c1ccc(-c2nc3c(cc(N(c4ccc(-n5c6ccccc6c6ccccc65)cc4)c4ccc5oc6ccccc6c5c4)c4ccccc43)o2)cc1 ; c1ccc(-c2nc3cc(N(c4ccc5oc6ccccc6c5c4)c4ccc5c6ccccc6n(-c6ccccc6)c5c4)c4ccccc4c3o2)cc1 ; c1ccc(-c2nc3c(cc(N(c4ccc5c(c4)oc4ccccc45)c4ccc5c6ccccc6n(-c6ccccc6)c5c4)c4ccccc43)o2)cc1]

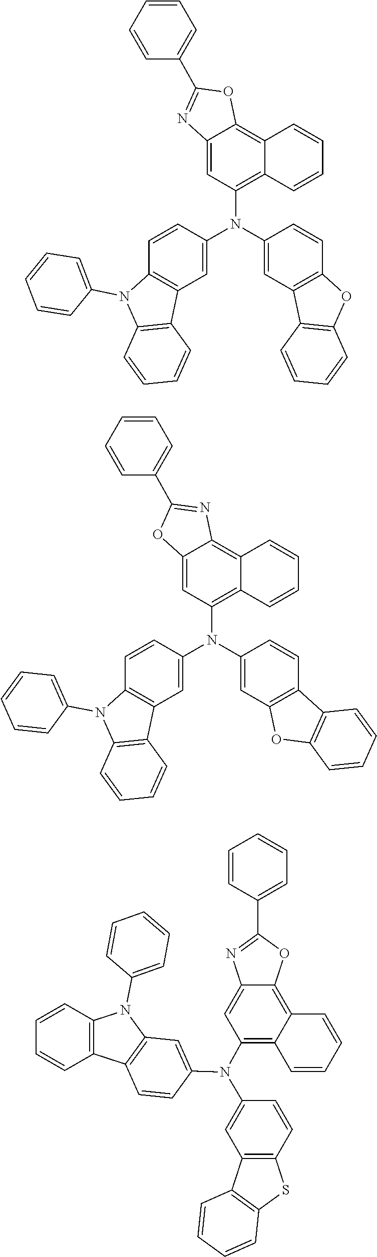

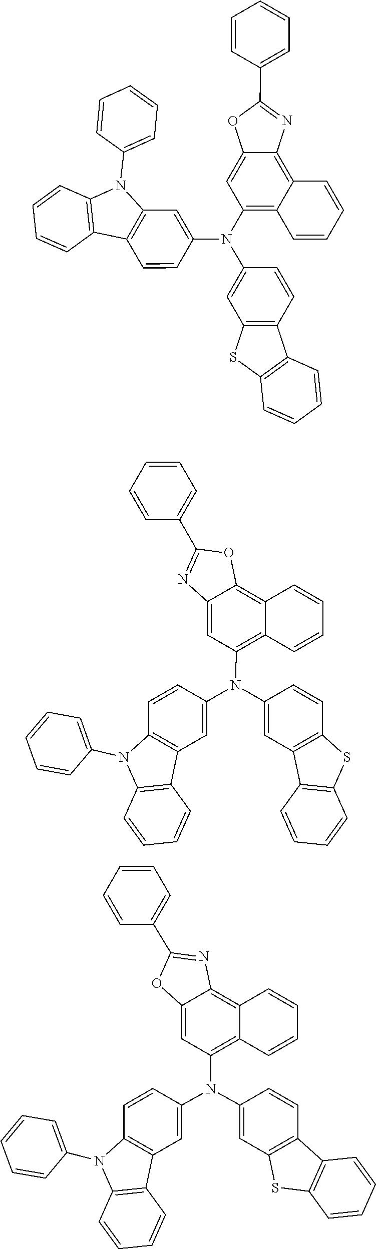

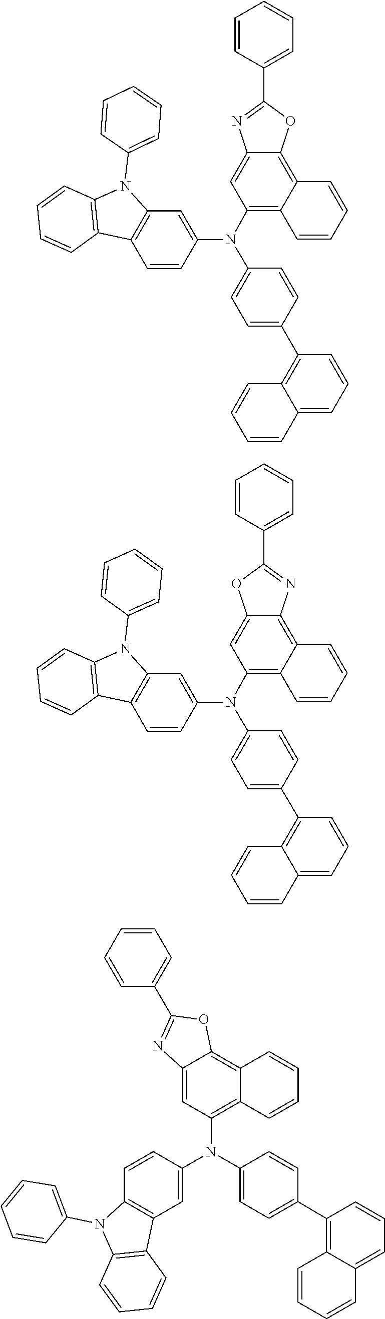

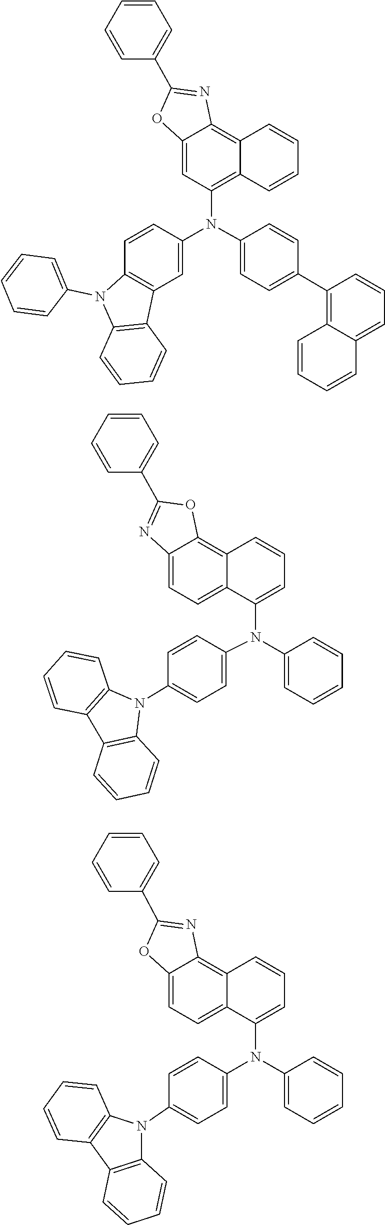

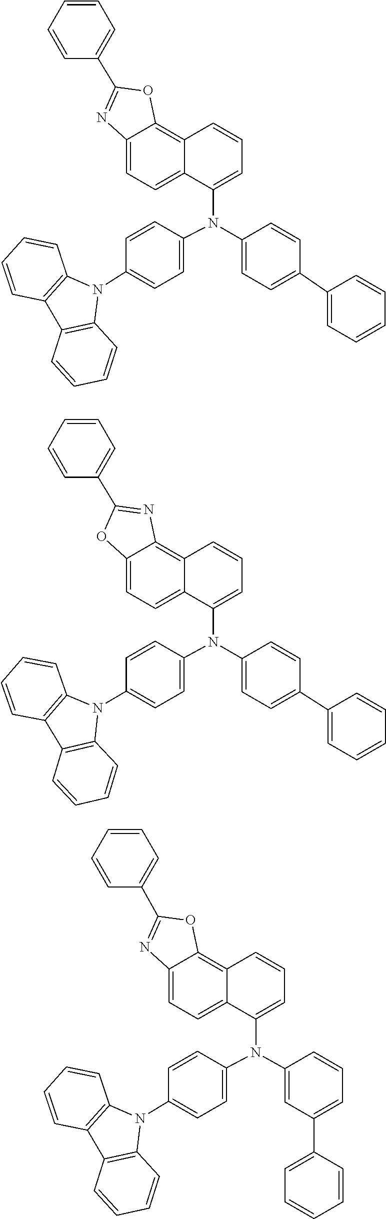

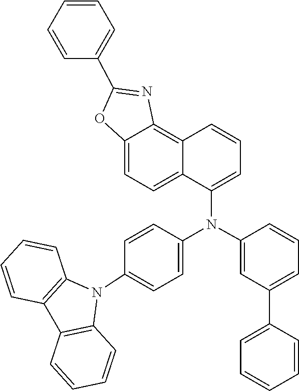 [SMILES: c1ccc(-c2cccc(N(c3ccc(-n4c5ccccc5c5ccccc54)cc3)c3cccc4c3ccc3oc(-c5ccccc5)nc34)c2)cc1]

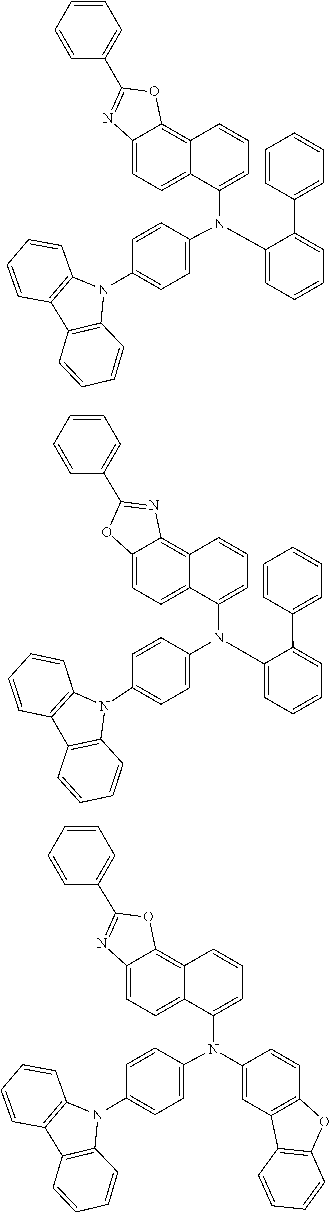

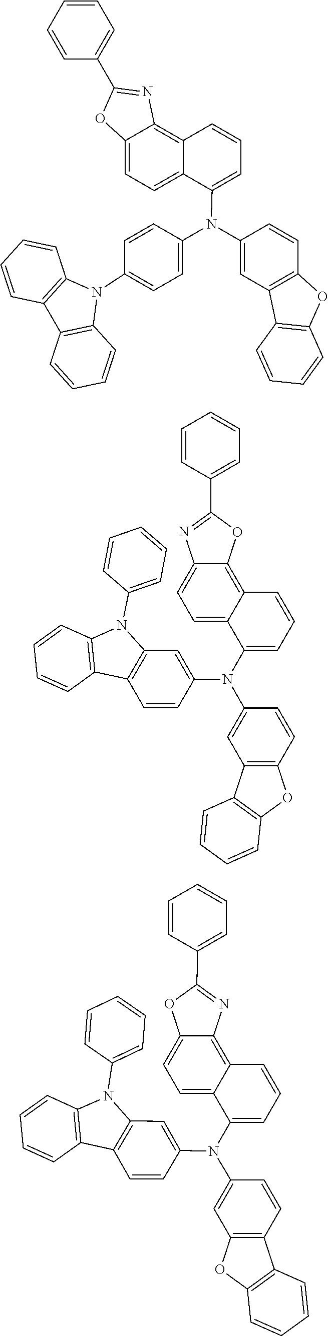

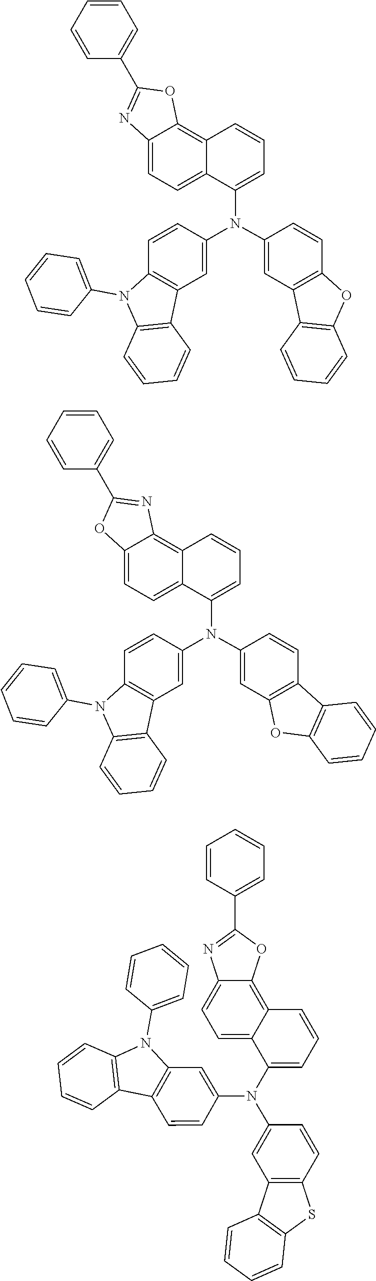 [SMILES: c1ccc(-c2nc3ccc4c(N(c5ccc6oc7ccccc7c6c5)c5ccc6c(c5)c5ccccc5n6-c5ccccc5)cccc4c3o2)cc1 ; c1ccc(-c2nc3c(ccc4c(N(c5ccc6c(c5)oc5ccccc56)c5ccc6c(c5)c5ccccc5n6-c5ccccc5)cccc43)o2)cc1 ; c1ccc(-c2nc3ccc4c(N(c5ccc6sc7ccccc7c6c5)c5ccc6c7ccccc7n(-c7ccccc7)c6c5)cccc4c3o2)cc1]

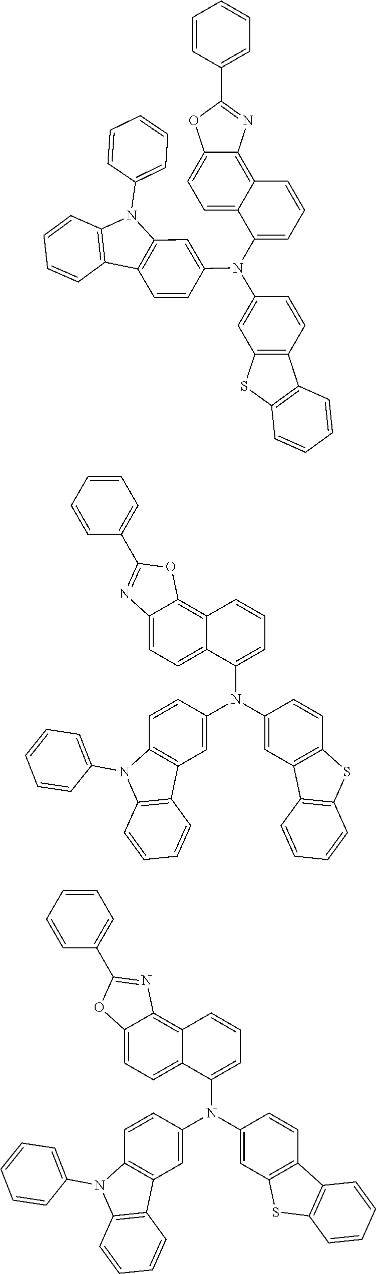 [SMILES: c1ccc(-c2nc3c(ccc4c(N(c5ccc6c(c5)sc5ccccc56)c5ccc6c7ccccc7n(-c7ccccc7)c6c5)cccc43)o2)cc1 ; c1ccc(-c2nc3ccc4c(N(c5ccc6sc7ccccc7c6c5)c5ccc6c(c5)c5ccccc5n6-c5ccccc5)cccc4c3o2)cc1 ; c1ccc(-c2nc3c(ccc4c(N(c5ccc6c(c5)sc5ccccc56)c5ccc6c(c5)c5ccccc5n6-c5ccccc5)cccc43)o2)cc1]

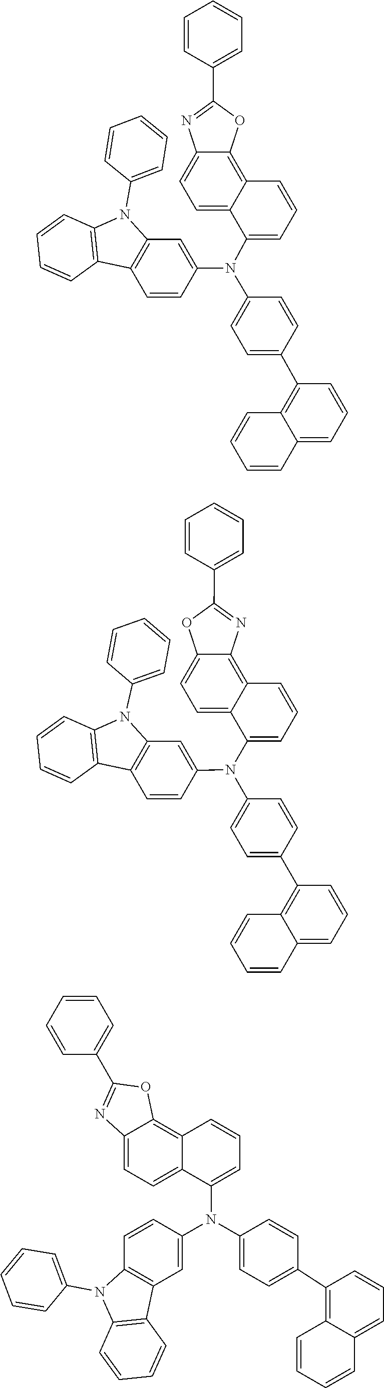

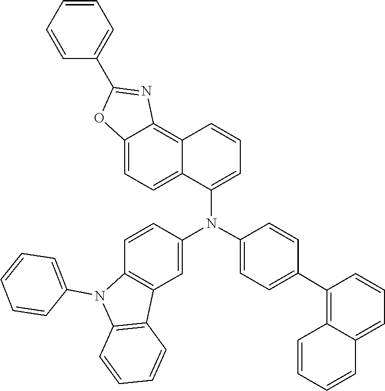 [SMILES: c1ccc(-c2nc3c(ccc4c(N(c5ccc(-c6cccc7ccccc67)cc5)c5ccc6c(c5)c5ccccc5n6-c5ccccc5)cccc43)o2)cc1]

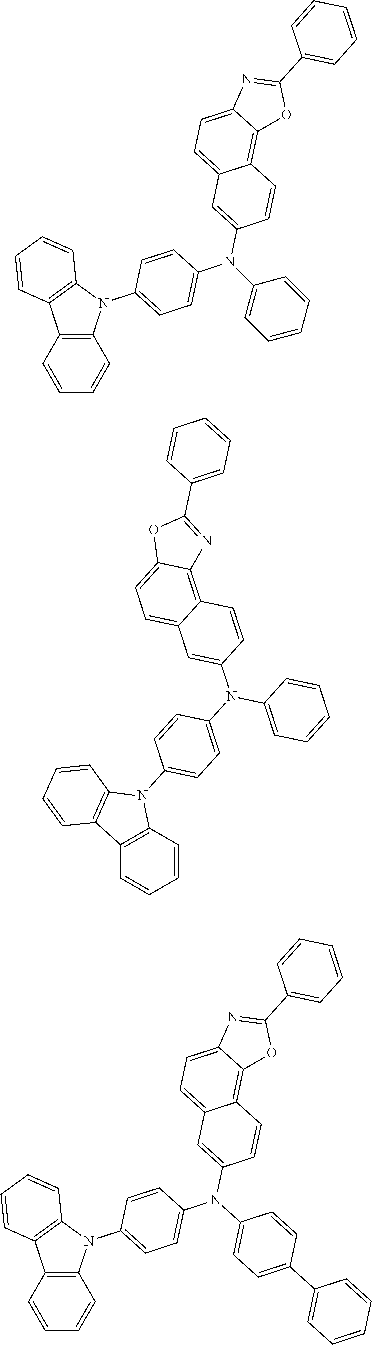 [SMILES: c1ccc(-c2nc3ccc4cc(N(c5ccccc5)c5ccc(-n6c7ccccc7c7ccccc76)cc5)ccc4c3o2)cc1 ; c1ccc(-c2nc3c(ccc4cc(N(c5ccccc5)c5ccc(-n6c7ccccc7c7ccccc76)cc5)ccc43)o2)cc1 ; c1ccc(-c2ccc(N(c3ccc(-n4c5ccccc5c5ccccc54)cc3)c3ccc4c(ccc5nc(-c6ccccc6)oc54)c3)cc2)cc1]

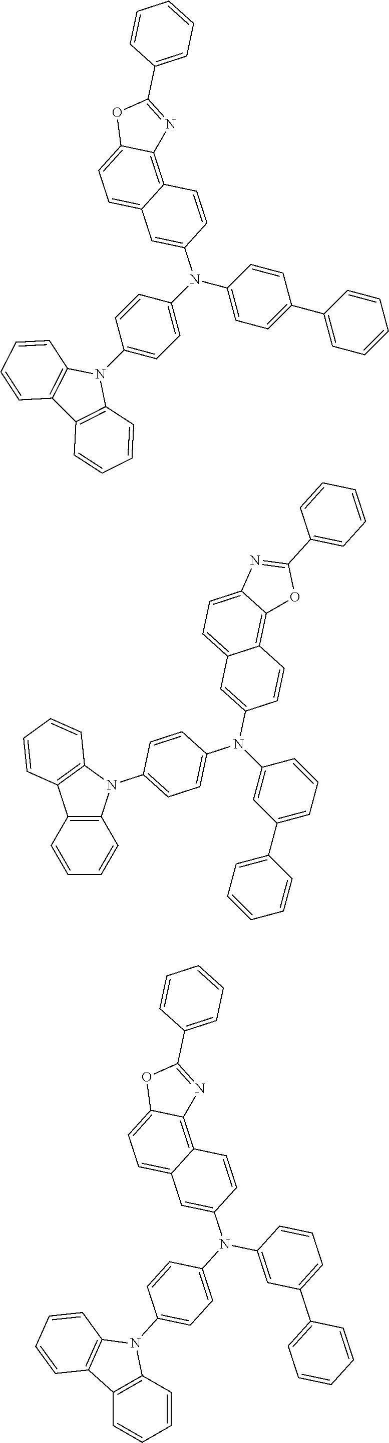 [SMILES: c1ccc(-c2ccc(N(c3ccc(-n4c5ccccc5c5ccccc54)cc3)c3ccc4c(ccc5oc(-c6ccccc6)nc54)c3)cc2)cc1 ; c1ccc(-c2cccc(N(c3ccc(-n4c5ccccc5c5ccccc54)cc3)c3ccc4c(ccc5nc(-c6ccccc6)oc54)c3)c2)cc1 ; c1ccc(-c2cccc(N(c3ccc(-n4c5ccccc5c5ccccc54)cc3)c3ccc4c(ccc5oc(-c6ccccc6)nc54)c3)c2)cc1]

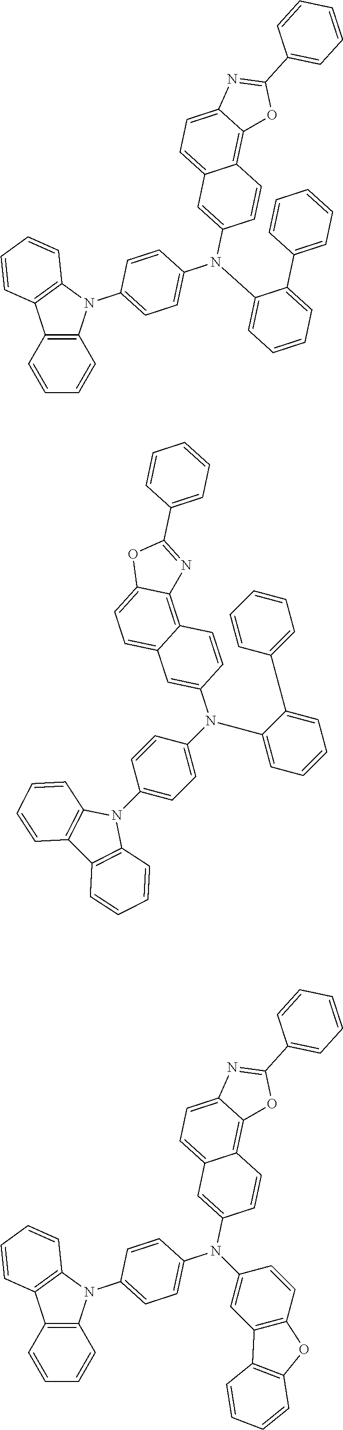 [SMILES: c1ccc(-c2nc3ccc4cc(N(c5ccc(-n6c7ccccc7c7ccccc76)cc5)c5ccccc5-c5ccccc5)ccc4c3o2)cc1 ; c1ccc(-c2nc3c(ccc4cc(N(c5ccc(-n6c7ccccc7c7ccccc76)cc5)c5ccccc5-c5ccccc5)ccc43)o2)cc1 ; c1ccc(-c2nc3ccc4cc(N(c5ccc(-n6c7ccccc7c7ccccc76)cc5)c5ccc6oc7ccccc7c6c5)ccc4c3o2)cc1]

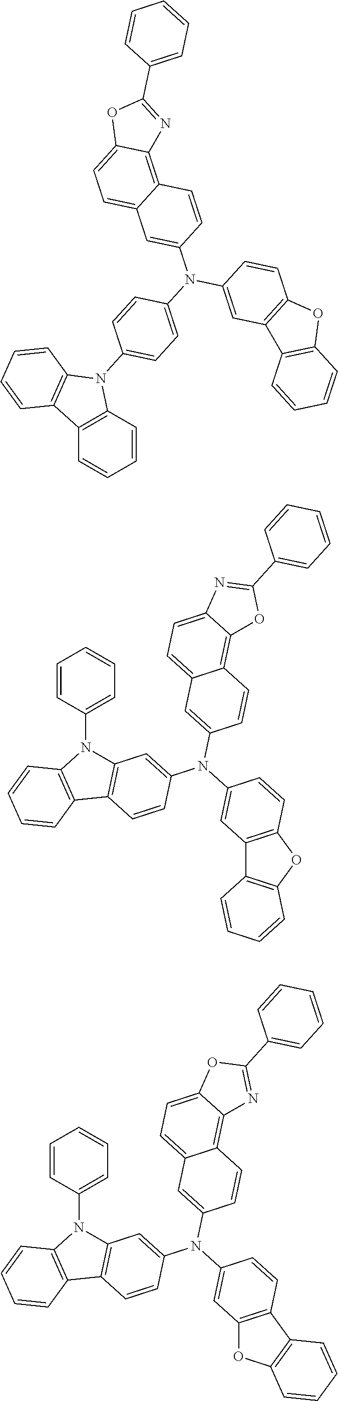 [SMILES: c1ccc(-c2nc3c(ccc4cc(N(c5ccc(-n6c7ccccc7c7ccccc76)cc5)c5ccc6oc7ccccc7c6c5)ccc43)o2)cc1 ; c1ccc(-c2nc3ccc4cc(N(c5ccc6oc7ccccc7c6c5)c5ccc6c7ccccc7n(-c7ccccc7)c6c5)ccc4c3o2)cc1 ; c1ccc(-c2nc3c(ccc4cc(N(c5ccc6c(c5)oc5ccccc56)c5ccc6c7ccccc7n(-c7ccccc7)c6c5)ccc43)o2)cc1]

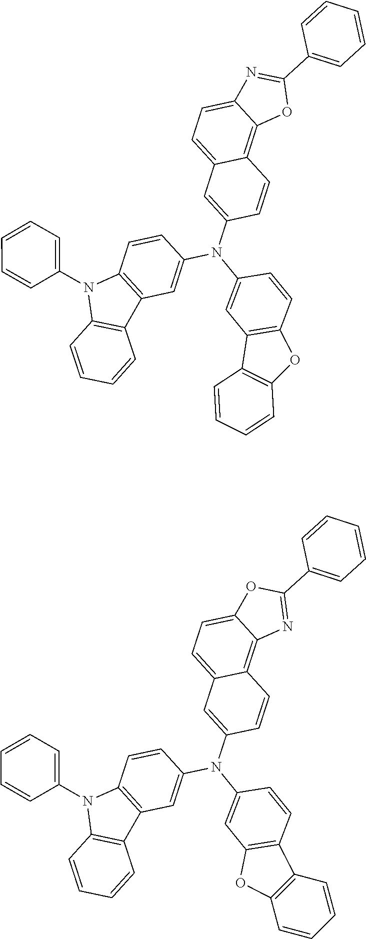 [SMILES: c1ccc(-c2nc3ccc4cc(N(c5ccc6oc7ccccc7c6c5)c5ccc6c(c5)c5ccccc5n6-c5ccccc5)ccc4c3o2)cc1 ; c1ccc(-c2nc3c(ccc4cc(N(c5ccc6c(c5)oc5ccccc56)c5ccc6c(c5)c5ccccc5n6-c5ccccc5)ccc43)o2)cc1]

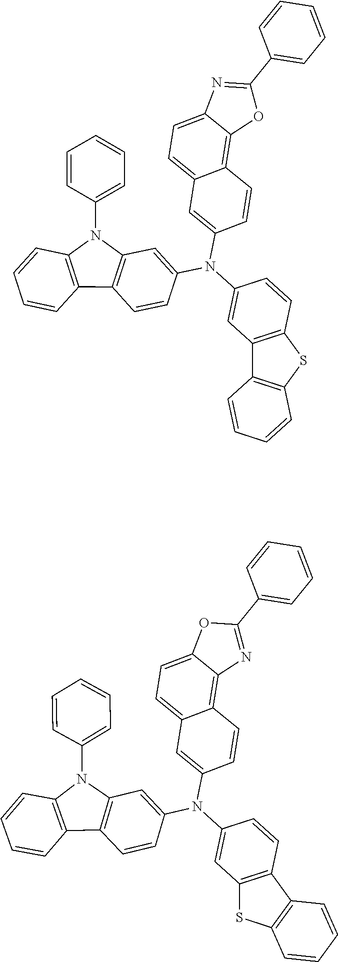 [SMILES: c1ccc(-c2nc3ccc4cc(N(c5ccc6sc7ccccc7c6c5)c5ccc6c7ccccc7n(-c7ccccc7)c6c5)ccc4c3o2)cc1 ; c1ccc(-c2nc3c(ccc4cc(N(c5ccc6c(c5)sc5ccccc56)c5ccc6c7ccccc7n(-c7ccccc7)c6c5)ccc43)o2)cc1]

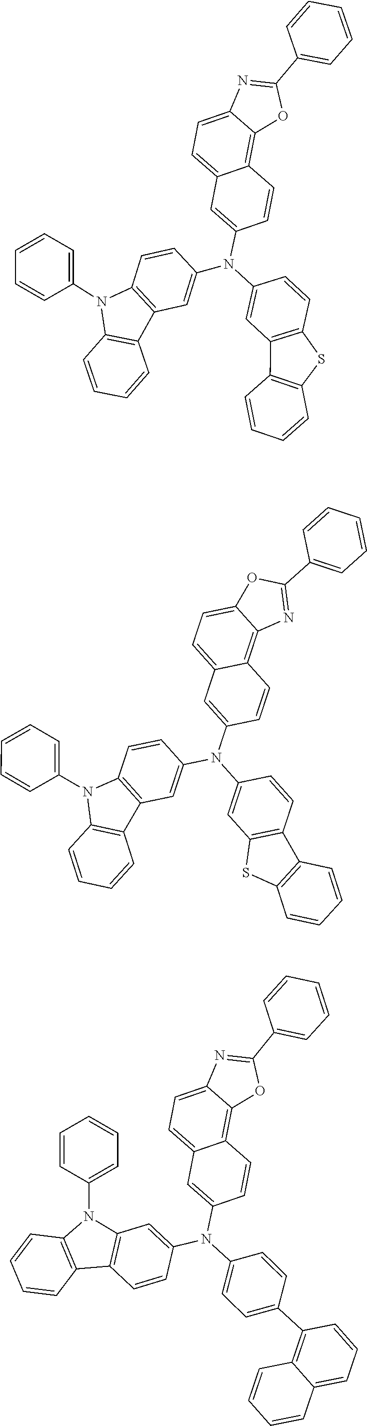 [SMILES: c1ccc(-c2nc3ccc4cc(N(c5ccc6sc7ccccc7c6c5)c5ccc6c(c5)c5ccccc5n6-c5ccccc5)ccc4c3o2)cc1 ; c1ccc(-c2nc3c(ccc4cc(N(c5ccc6c(c5)sc5ccccc56)c5ccc6c(c5)c5ccccc5n6-c5ccccc5)ccc43)o2)cc1 ; c1ccc(-c2nc3ccc4cc(N(c5ccc(-c6cccc7ccccc67)cc5)c5ccc6c7ccccc7n(-c7ccccc7)c6c5)ccc4c3o2)cc1]

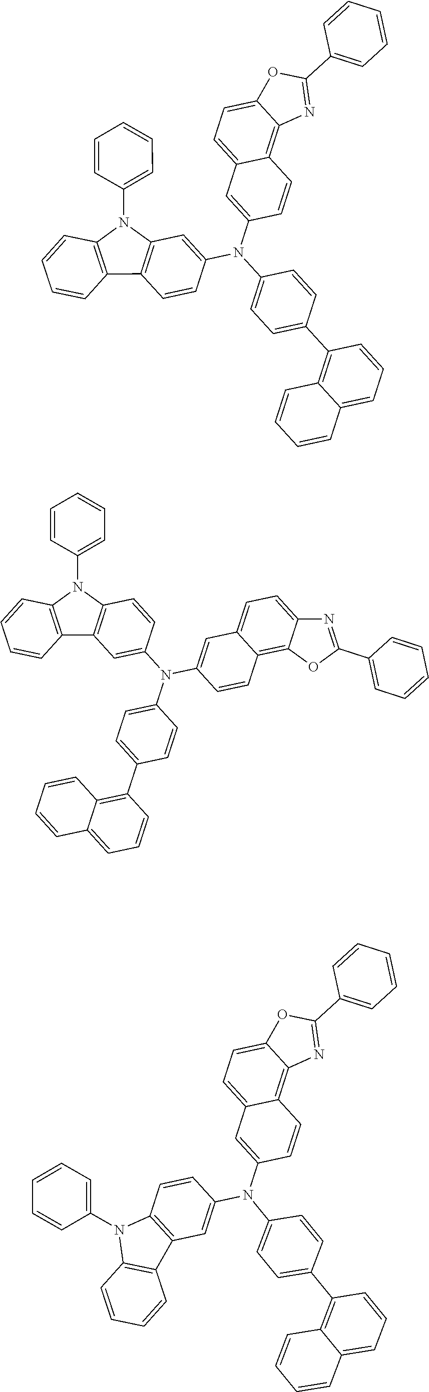 [SMILES: c1ccc(-c2nc3c(ccc4cc(N(c5ccc(-c6cccc7ccccc67)cc5)c5ccc6c7ccccc7n(-c7ccccc7)c6c5)ccc43)o2)cc1 ; c1ccc(-c2nc3ccc4cc(N(c5ccc(-c6cccc7ccccc67)cc5)c5ccc6c(c5)c5ccccc5n6-c5ccccc5)ccc4c3o2)cc1 ; c1ccc(-c2nc3c(ccc4cc(N(c5ccc(-c6cccc7ccccc67)cc5)c5ccc6c(c5)c5ccccc5n6-c5ccccc5)ccc43)o2)cc1]

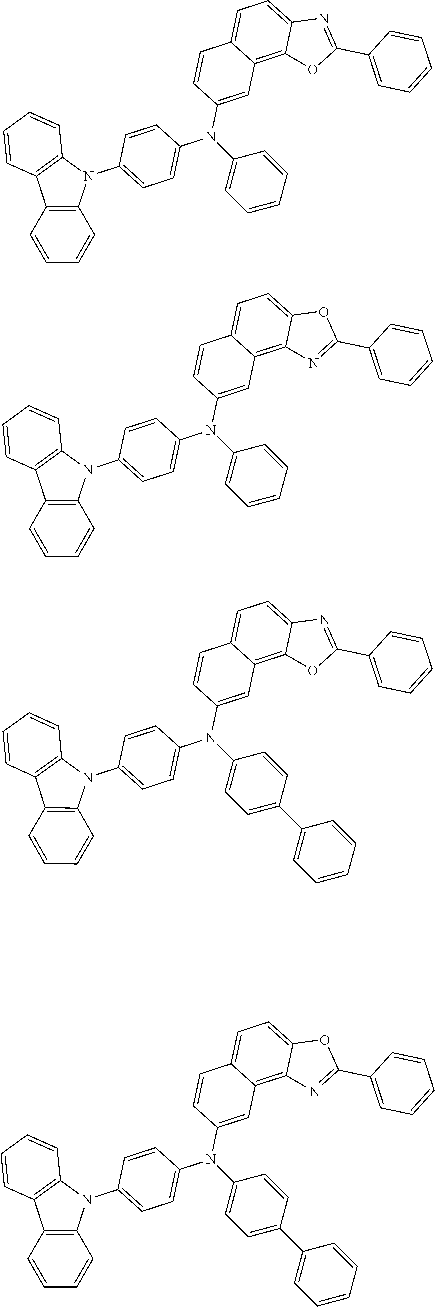 [SMILES: c1ccc(-c2nc3ccc4ccc(N(c5ccccc5)c5ccc(-n6c7ccccc7c7ccccc76)cc5)cc4c3o2)cc1 ; c1ccc(-c2nc3c(ccc4ccc(N(c5ccccc5)c5ccc(-n6c7ccccc7c7ccccc76)cc5)cc43)o2)cc1 ; c1ccc(-c2ccc(N(c3ccc(-n4c5ccccc5c5ccccc54)cc3)c3ccc4ccc5nc(-c6ccccc6)oc5c4c3)cc2)cc1 ; c1ccc(-c2ccc(N(c3ccc(-n4c5ccccc5c5ccccc54)cc3)c3ccc4ccc5oc(-c6ccccc6)nc5c4c3)cc2)cc1]

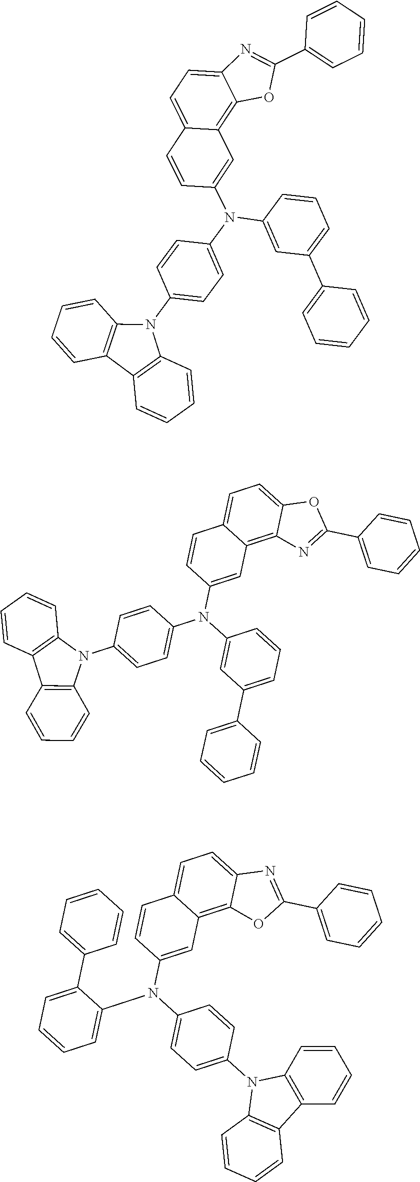 [SMILES: c1ccc(-c2cccc(N(c3ccc(-n4c5ccccc5c5ccccc54)cc3)c3ccc4ccc5nc(-c6ccccc6)oc5c4c3)c2)cc1 ; c1ccc(-c2cccc(N(c3ccc(-n4c5ccccc5c5ccccc54)cc3)c3ccc4ccc5oc(-c6ccccc6)nc5c4c3)c2)cc1 ; c1ccc(-c2nc3ccc4ccc(N(c5ccc(-n6c7ccccc7c7ccccc76)cc5)c5ccccc5-c5ccccc5)cc4c3o2)cc1]

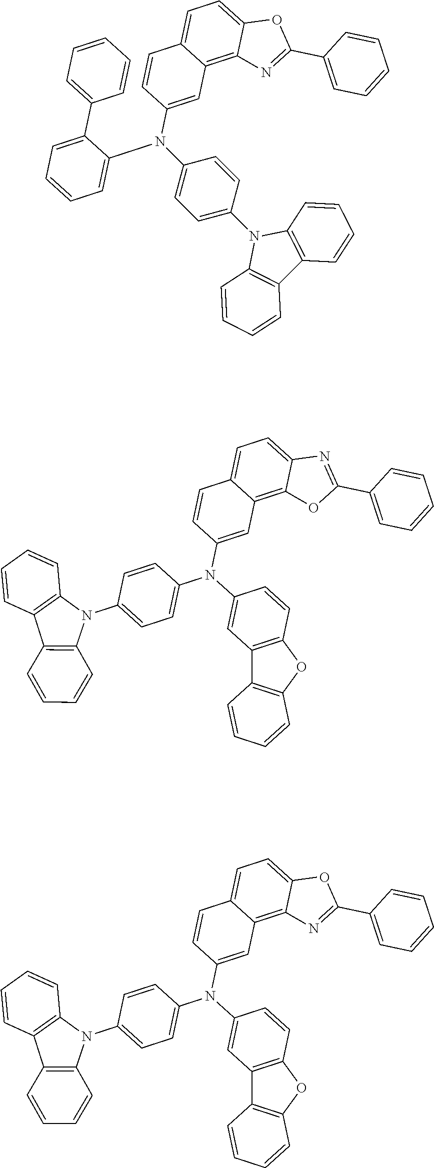 [SMILES: c1ccc(-c2nc3c(ccc4ccc(N(c5ccc(-n6c7ccccc7c7ccccc76)cc5)c5ccccc5-c5ccccc5)cc43)o2)cc1 ; c1ccc(-c2nc3ccc4ccc(N(c5ccc(-n6c7ccccc7c7ccccc76)cc5)c5ccc6oc7ccccc7c6c5)cc4c3o2)cc1 ; c1ccc(-c2nc3c(ccc4ccc(N(c5ccc(-n6c7ccccc7c7ccccc76)cc5)c5ccc6oc7ccccc7c6c5)cc43)o2)cc1]

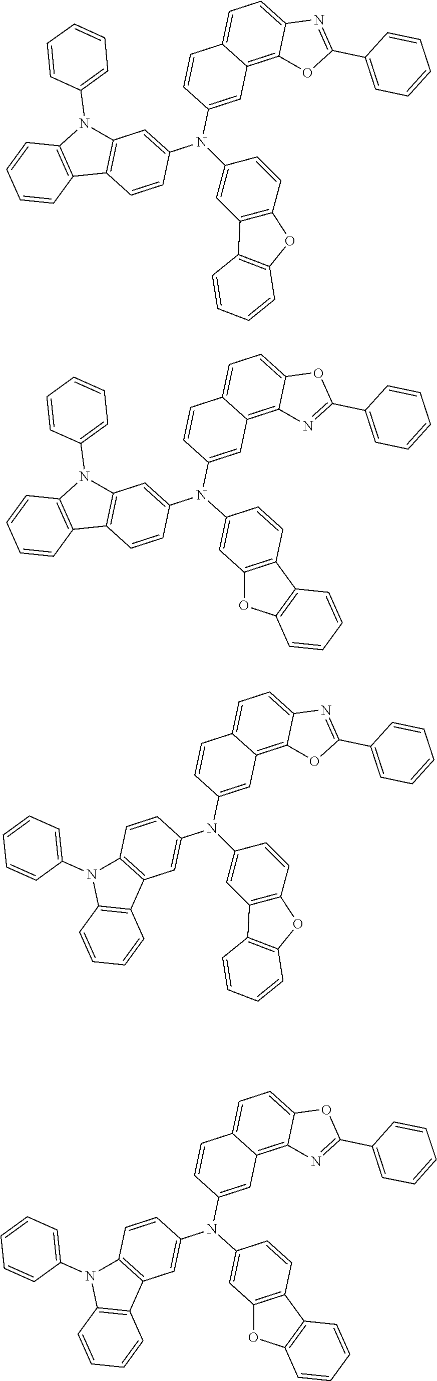

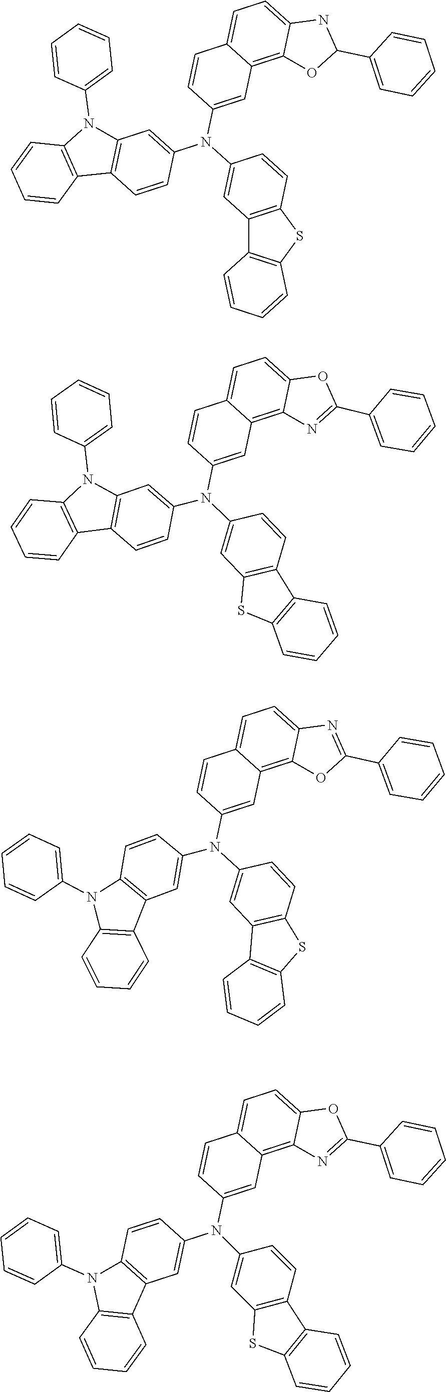

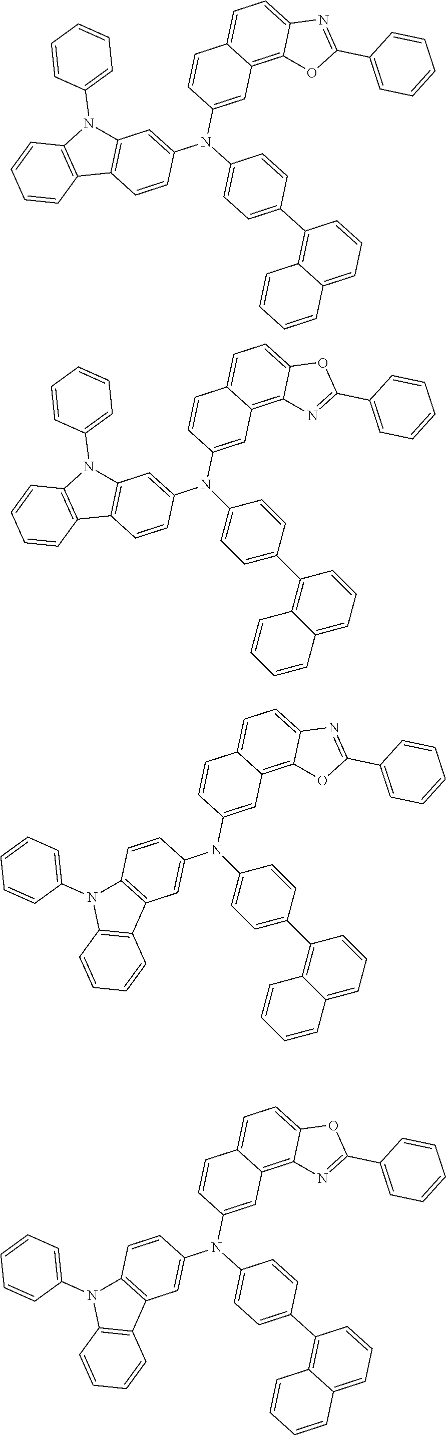

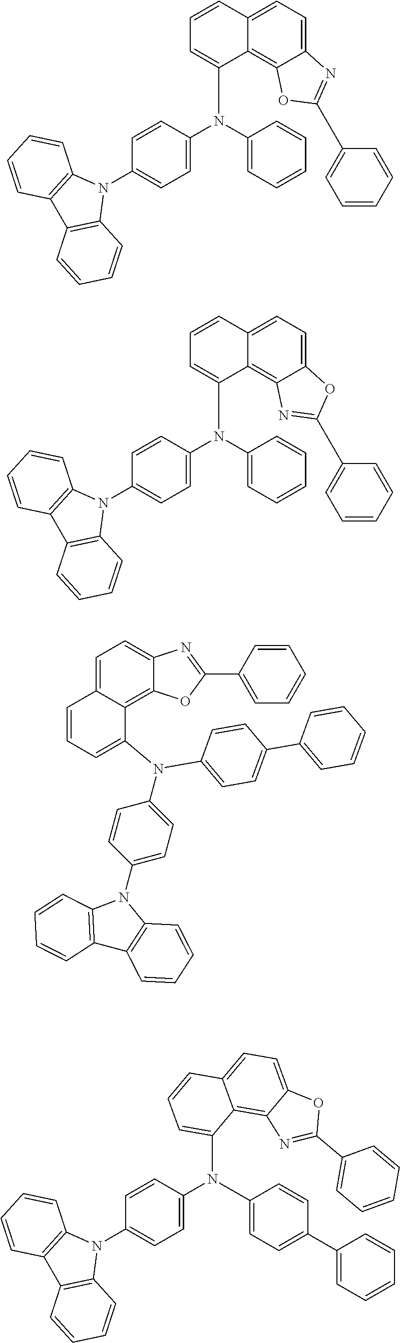 [SMILES: c1ccc(-c2nc3ccc4cccc(N(c5ccccc5)c5ccc(-n6c7ccccc7c7ccccc76)cc5)c4c3o2)cc1 ; c1ccc(-c2nc3c(ccc4cccc(N(c5ccccc5)c5ccc(-n6c7ccccc7c7ccccc76)cc5)c43)o2)cc1 ; c1ccc(-c2ccc(N(c3ccc(-n4c5ccccc5c5ccccc54)cc3)c3cccc4ccc5nc(-c6ccccc6)oc5c34)cc2)cc1 ; c1ccc(-c2ccc(N(c3ccc(-n4c5ccccc5c5ccccc54)cc3)c3cccc4ccc5oc(-c6ccccc6)nc5c34)cc2)cc1]

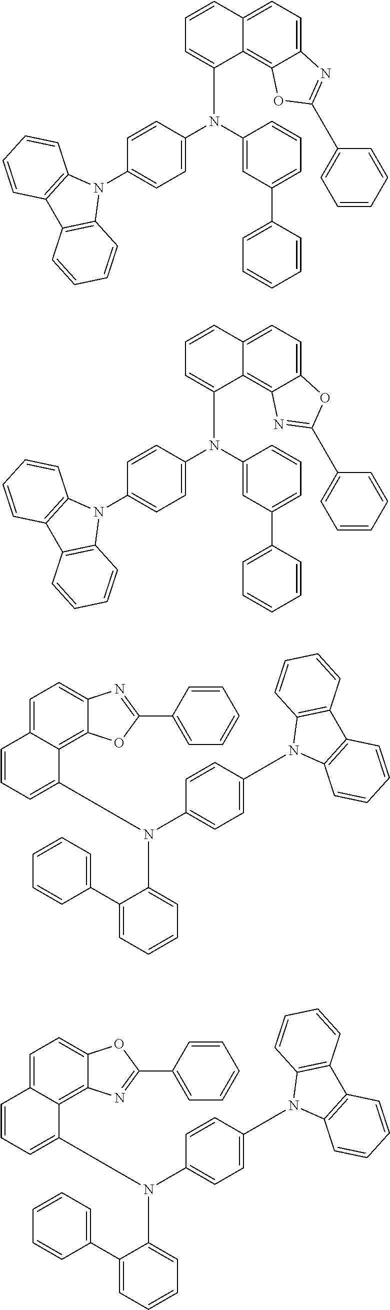

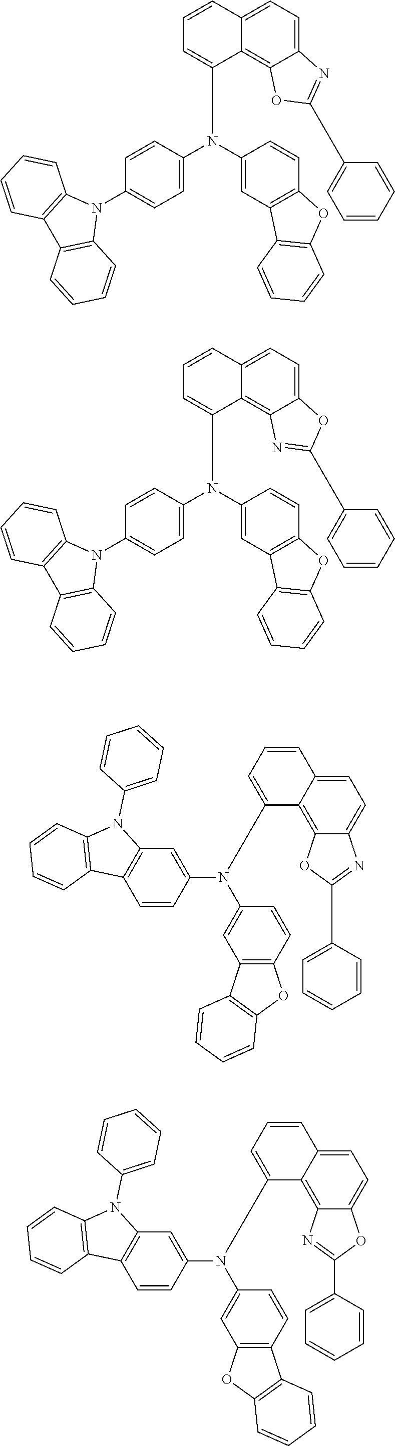 [SMILES: c1ccc(-c2nc3ccc4cccc(N(c5ccc(-n6c7ccccc7c7ccccc76)cc5)c5ccc6oc7ccccc7c6c5)c4c3o2)cc1 ; c1ccc(-c2nc3c(ccc4cccc(N(c5ccc(-n6c7ccccc7c7ccccc76)cc5)c5ccc6oc7ccccc7c6c5)c43)o2)cc1 ; c1ccc(-c2nc3ccc4cccc(N(c5ccc6oc7ccccc7c6c5)c5ccc6c7ccccc7n(-c7ccccc7)c6c5)c4c3o2)cc1 ; c1ccc(-c2nc3c(ccc4cccc(N(c5ccc6c(c5)oc5ccccc56)c5ccc6c7ccccc7n(-c7ccccc7)c6c5)c43)o2)cc1]

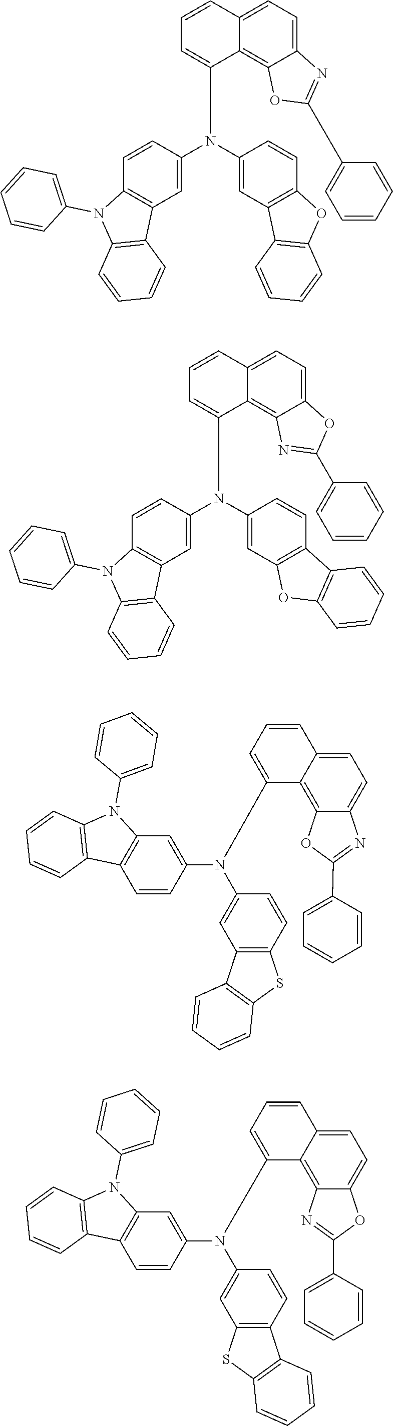 [SMILES: c1ccc(-c2nc3ccc4cccc(N(c5ccc6oc7ccccc7c6c5)c5ccc6c(c5)c5ccccc5n6-c5ccccc5)c4c3o2)cc1 ; c1ccc(-c2nc3c(ccc4cccc(N(c5ccc6c(c5)oc5ccccc56)c5ccc6c(c5)c5ccccc5n6-c5ccccc5)c43)o2)cc1 ; c1ccc(-c2nc3ccc4cccc(N(c5ccc6sc7ccccc7c6c5)c5ccc6c7ccccc7n(-c7ccccc7)c6c5)c4c3o2)cc1 ; c1ccc(-c2nc3c(ccc4cccc(N(c5ccc6c(c5)sc5ccccc56)c5ccc6c7ccccc7n(-c7ccccc7)c6c5)c43)o2)cc1]

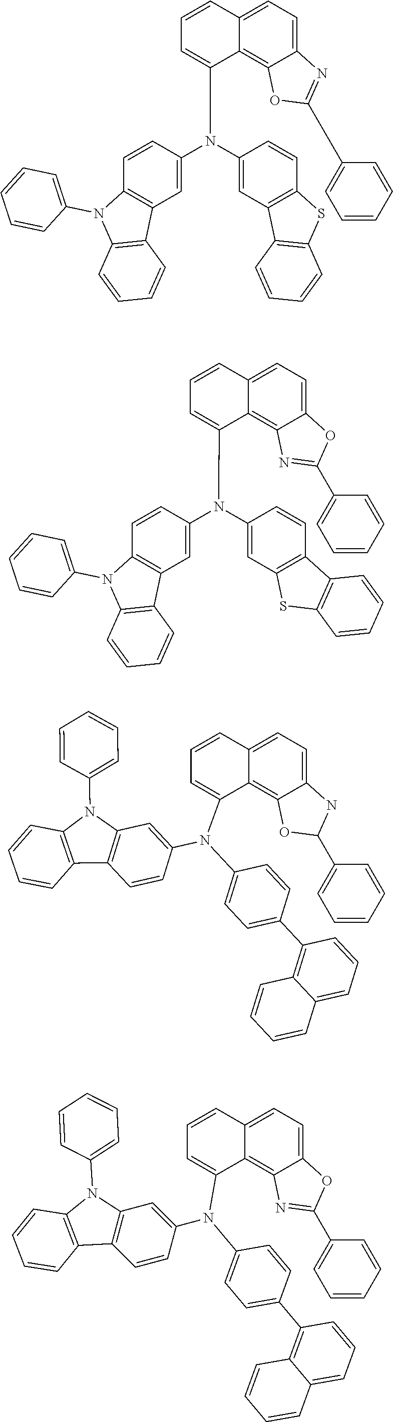

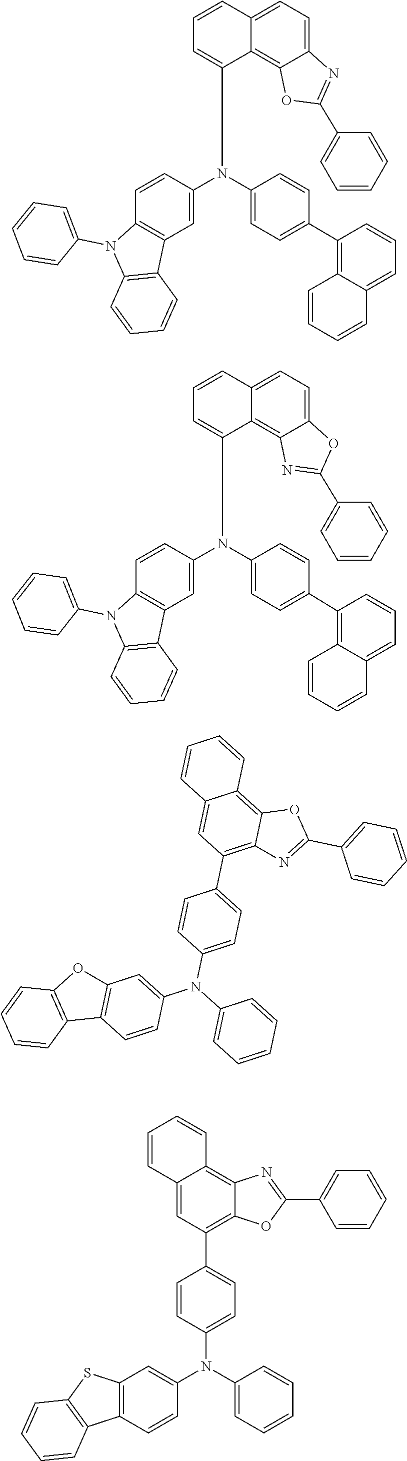 [SMILES: c1ccc(-c2nc3ccc4cccc(N(c5ccc(-c6cccc7ccccc67)cc5)c5ccc6c(c5)c5ccccc5n6-c5ccccc5)c4c3o2)cc1 ; c1ccc(-c2nc3c(ccc4cccc(N(c5ccc(-c6cccc7ccccc67)cc5)c5ccc6c(c5)c5ccccc5n6-c5ccccc5)c43)o2)cc1 ; c1ccc(-c2nc3c(-c4ccc(N(c5ccccc5)c5ccc6c(c5)oc5ccccc56)cc4)cc4ccccc4c3o2)cc1 ; c1ccc(-c2nc3c(o2)c(-c2ccc(N(c4ccccc4)c4ccc5c(c4)sc4ccccc45)cc2)cc2ccccc23)cc1]

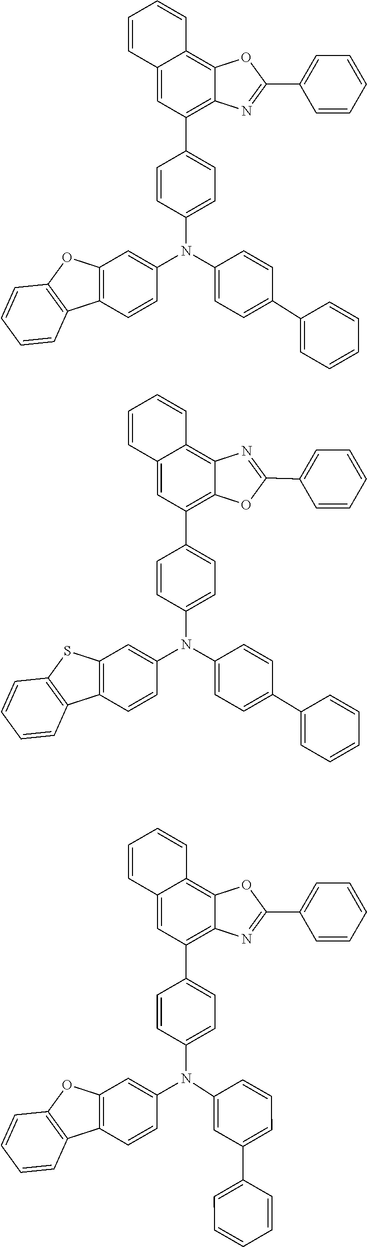 [SMILES: c1ccc(-c2ccc(N(c3ccc(-c4cc5ccccc5c5oc(-c6ccccc6)nc45)cc3)c3ccc4c(c3)oc3ccccc34)cc2)cc1 ; c1ccc(-c2ccc(N(c3ccc(-c4cc5ccccc5c5nc(-c6ccccc6)oc45)cc3)c3ccc4c(c3)sc3ccccc34)cc2)cc1 ; c1ccc(-c2cccc(N(c3ccc(-c4cc5ccccc5c5oc(-c6ccccc6)nc45)cc3)c3ccc4c(c3)oc3ccccc34)c2)cc1]

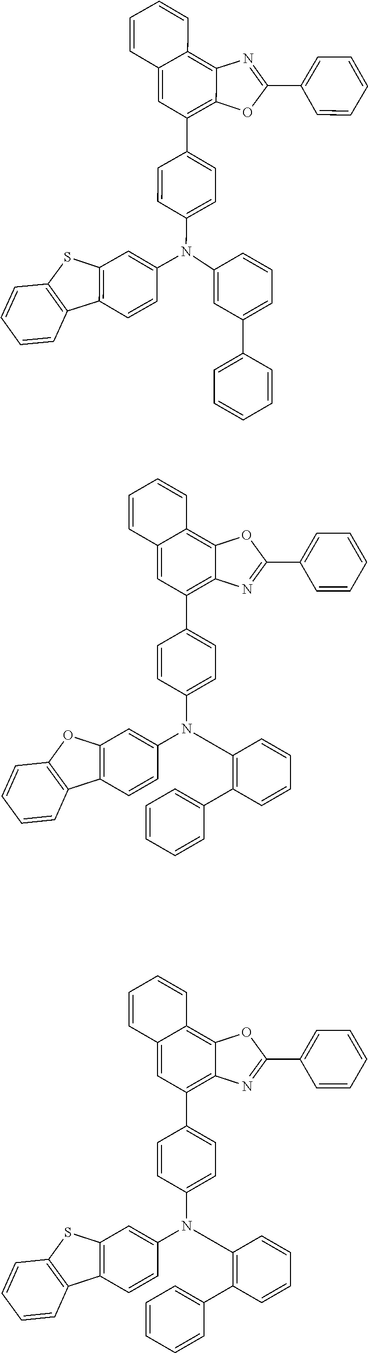 [SMILES: c1ccc(-c2cccc(N(c3ccc(-c4cc5ccccc5c5nc(-c6ccccc6)oc45)cc3)c3ccc4c(c3)sc3ccccc34)c2)cc1 ; c1ccc(-c2nc3c(-c4ccc(N(c5ccc6c(c5)oc5ccccc56)c5ccccc5-c5ccccc5)cc4)cc4ccccc4c3o2)cc1 ; c1ccc(-c2nc3c(-c4ccc(N(c5ccc6c(c5)sc5ccccc56)c5ccccc5-c5ccccc5)cc4)cc4ccccc4c3o2)cc1]

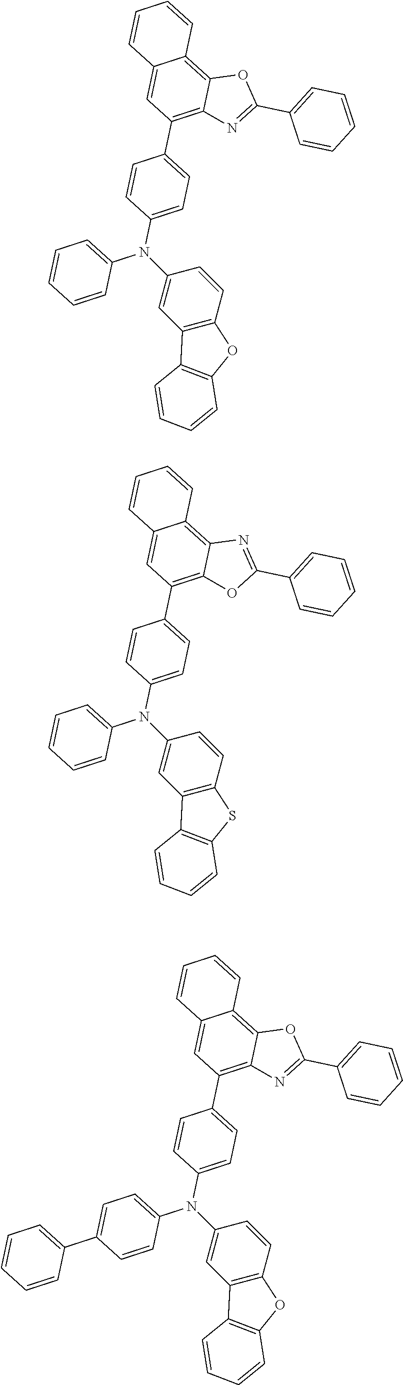

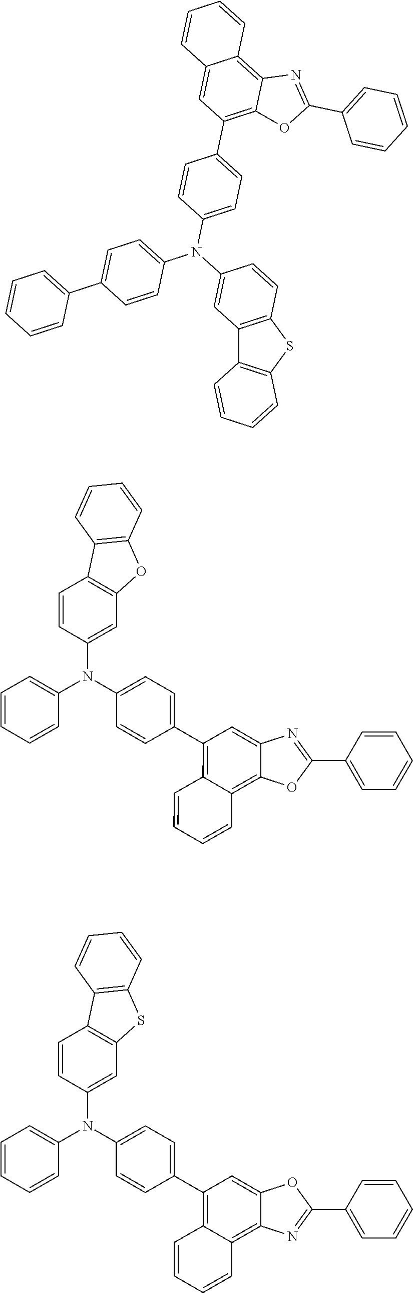 [SMILES: c1ccc(-c2ccc(N(c3ccc(-c4cc5ccccc5c5nc(-c6ccccc6)oc45)cc3)c3ccc4sc5ccccc5c4c3)cc2)cc1 ; c1ccc(-c2nc3cc(-c4ccc(N(c5ccccc5)c5ccc6c(c5)oc5ccccc56)cc4)c4ccccc4c3o2)cc1 ; c1ccc(-c2nc3c(cc(-c4ccc(N(c5ccccc5)c5ccc6c(c5)sc5ccccc56)cc4)c4ccccc43)o2)cc1]

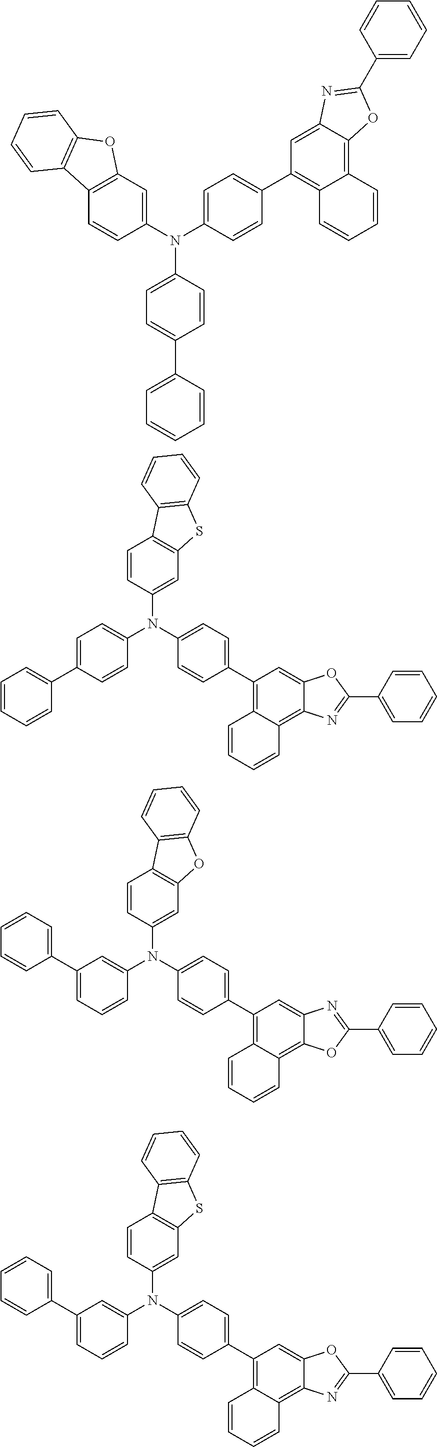

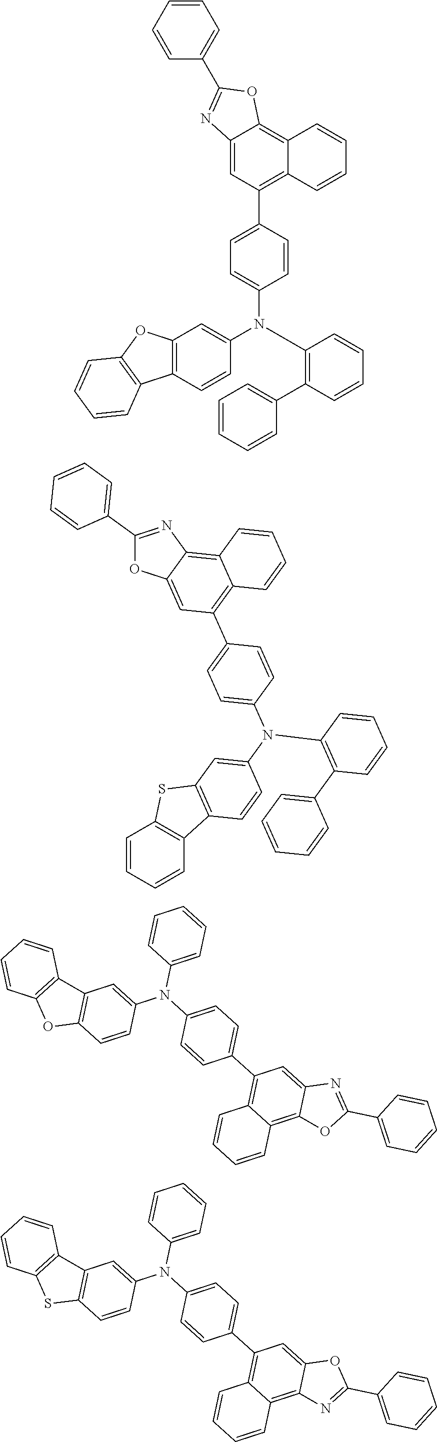

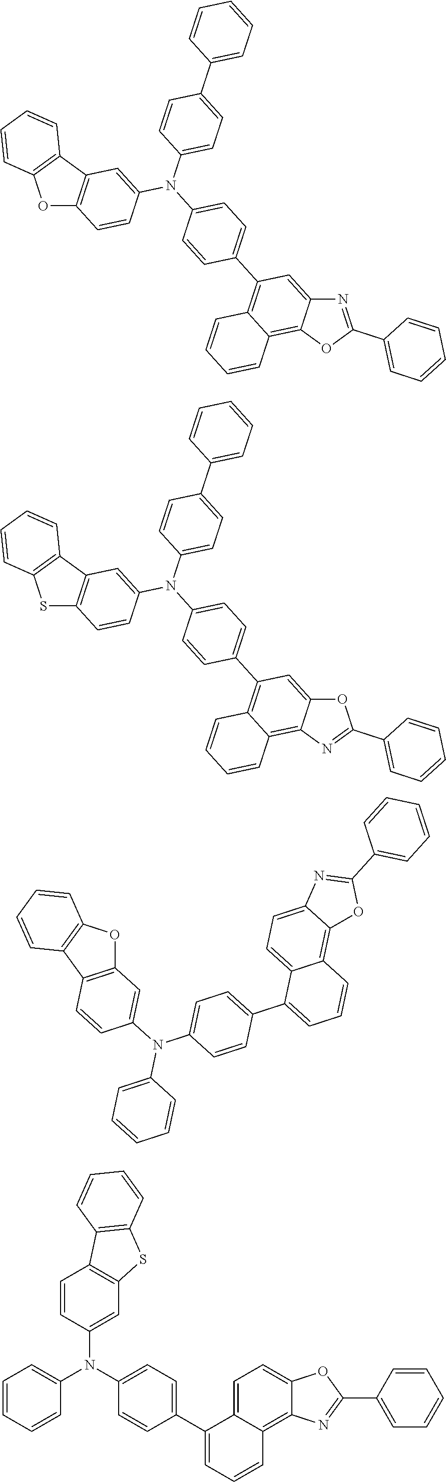

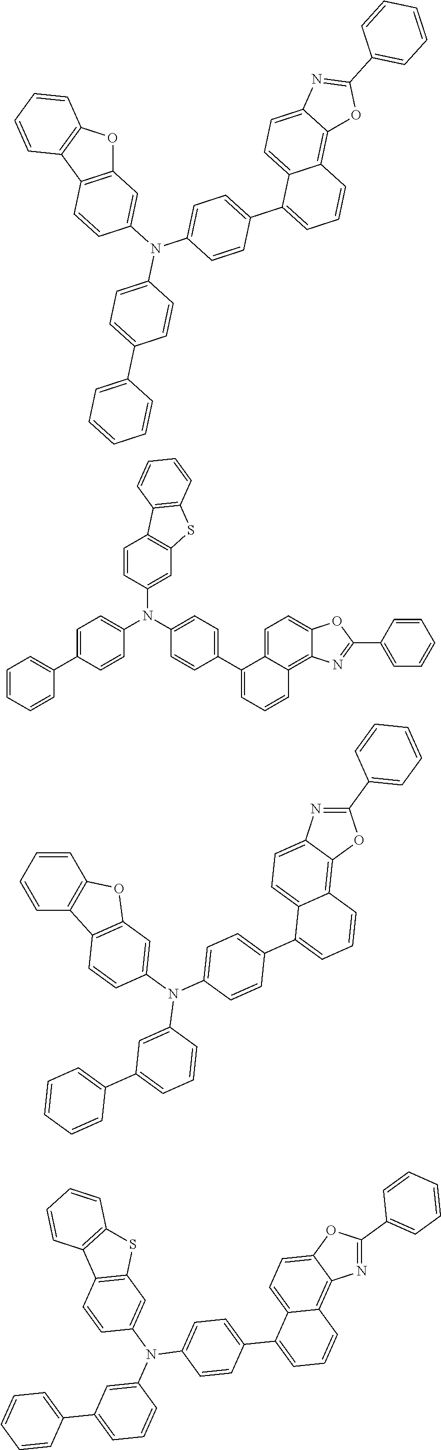

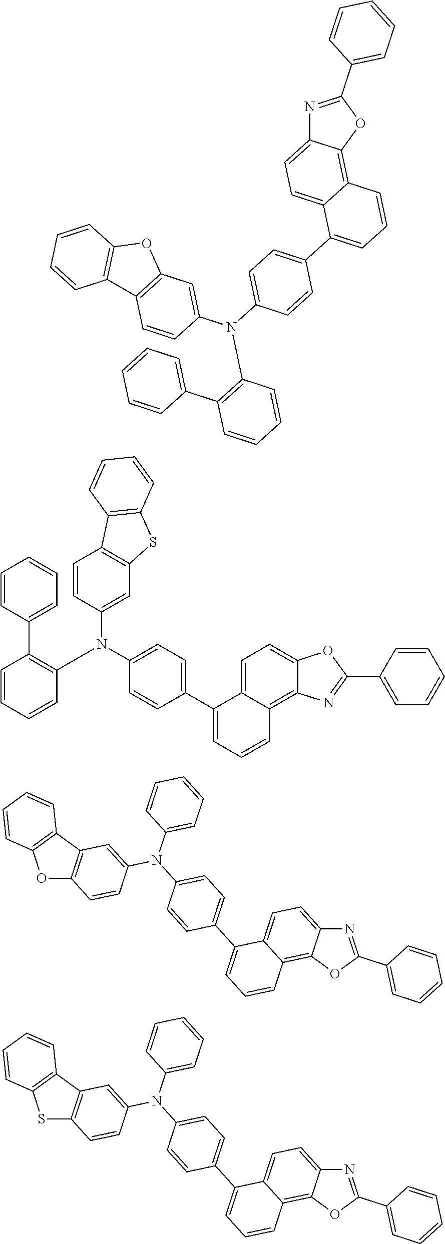 [SMILES: c1ccc(-c2nc3ccc4c(-c5ccc(N(c6ccc7c(c6)oc6ccccc67)c6ccccc6-c6ccccc6)cc5)cccc4c3o2)cc1 ; c1ccc(-c2nc3c(ccc4c(-c5ccc(N(c6ccc7c(c6)sc6ccccc67)c6ccccc6-c6ccccc6)cc5)cccc43)o2)cc1 ; c1ccc(-c2nc3ccc4c(-c5ccc(N(c6ccccc6)c6ccc7oc8ccccc8c7c6)cc5)cccc4c3o2)cc1 ; c1ccc(-c2nc3ccc4c(-c5ccc(N(c6ccccc6)c6ccc7sc8ccccc8c7c6)cc5)cccc4c3o2)cc1]

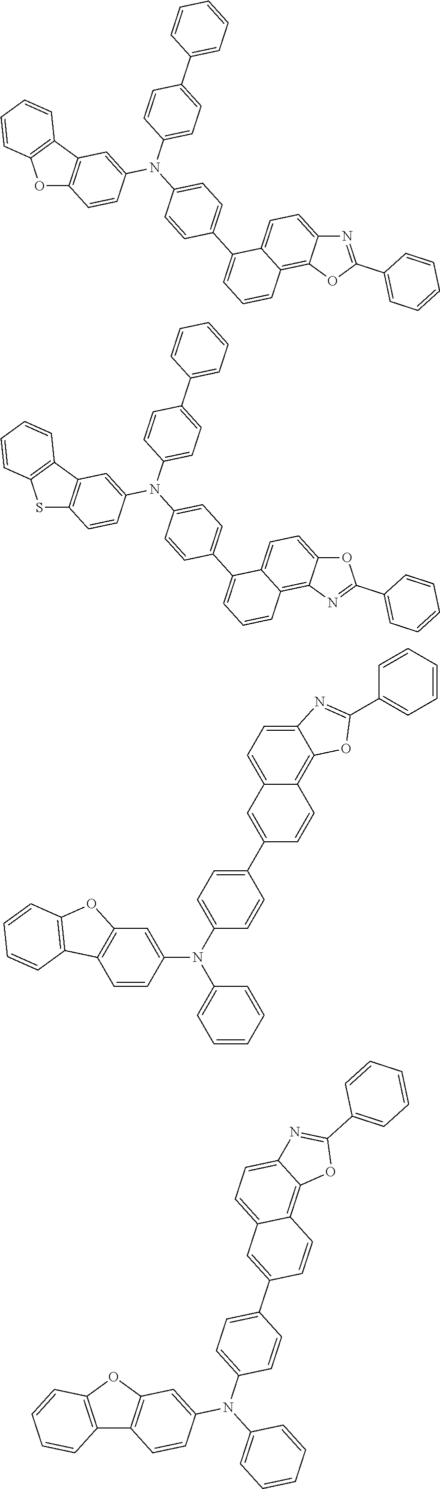

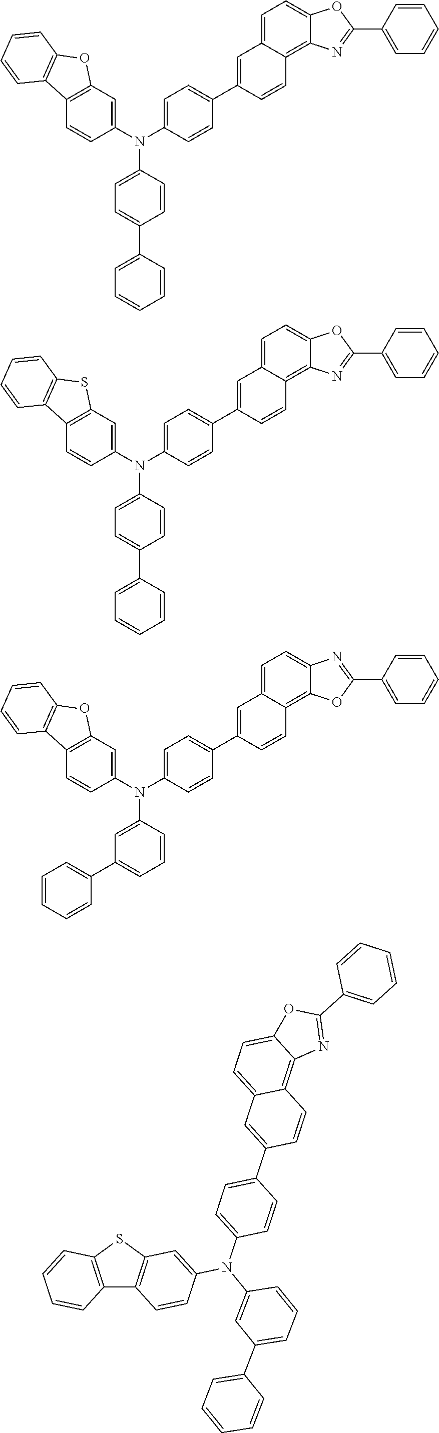 [SMILES: c1ccc(-c2ccc(N(c3ccc(-c4ccc5c(ccc6oc(-c7ccccc7)nc65)c4)cc3)c3ccc4c(c3)oc3ccccc34)cc2)cc1 ; c1ccc(-c2ccc(N(c3ccc(-c4ccc5c(ccc6oc(-c7ccccc7)nc65)c4)cc3)c3ccc4c(c3)sc3ccccc34)cc2)cc1 ; c1ccc(-c2cccc(N(c3ccc(-c4ccc5c(ccc6nc(-c7ccccc7)oc65)c4)cc3)c3ccc4c(c3)oc3ccccc34)c2)cc1 ; c1ccc(-c2cccc(N(c3ccc(-c4ccc5c(ccc6oc(-c7ccccc7)nc65)c4)cc3)c3ccc4c(c3)sc3ccccc34)c2)cc1]

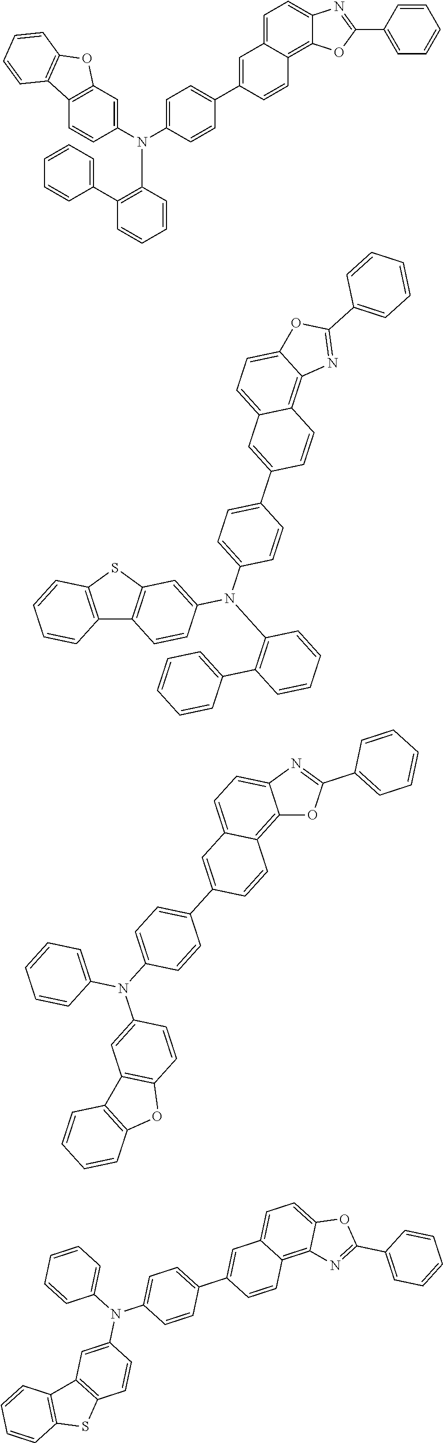 [SMILES: c1ccc(-c2nc3ccc4cc(-c5ccc(N(c6ccc7c(c6)oc6ccccc67)c6ccccc6-c6ccccc6)cc5)ccc4c3o2)cc1 ; c1ccc(-c2nc3c(ccc4cc(-c5ccc(N(c6ccc7c(c6)sc6ccccc67)c6ccccc6-c6ccccc6)cc5)ccc43)o2)cc1 ; c1ccc(-c2nc3ccc4cc(-c5ccc(N(c6ccccc6)c6ccc7oc8ccccc8c7c6)cc5)ccc4c3o2)cc1 ; c1ccc(-c2nc3c(ccc4cc(-c5ccc(N(c6ccccc6)c6ccc7sc8ccccc8c7c6)cc5)ccc43)o2)cc1]

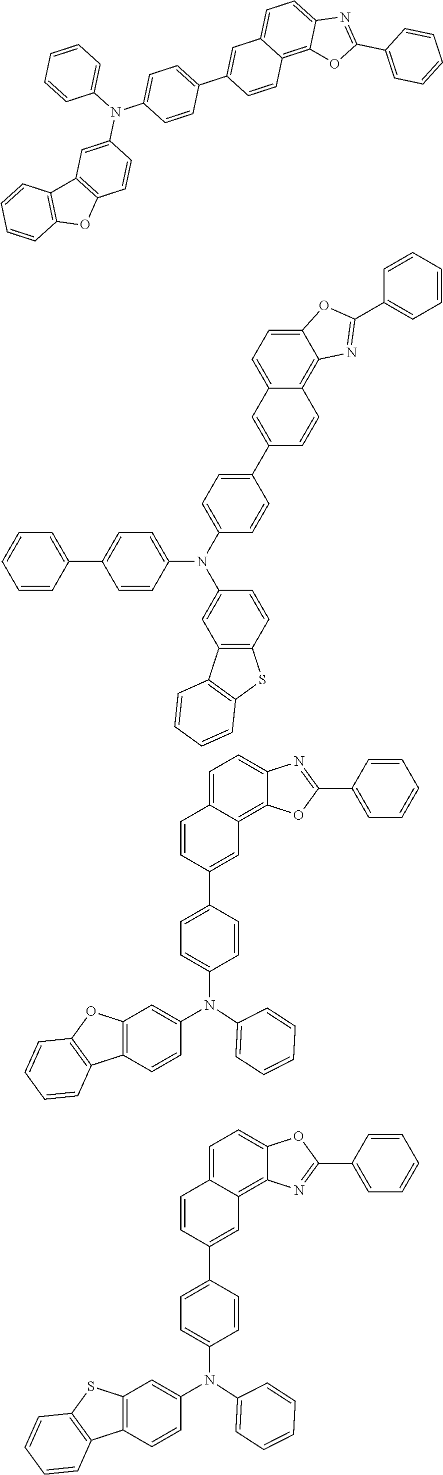

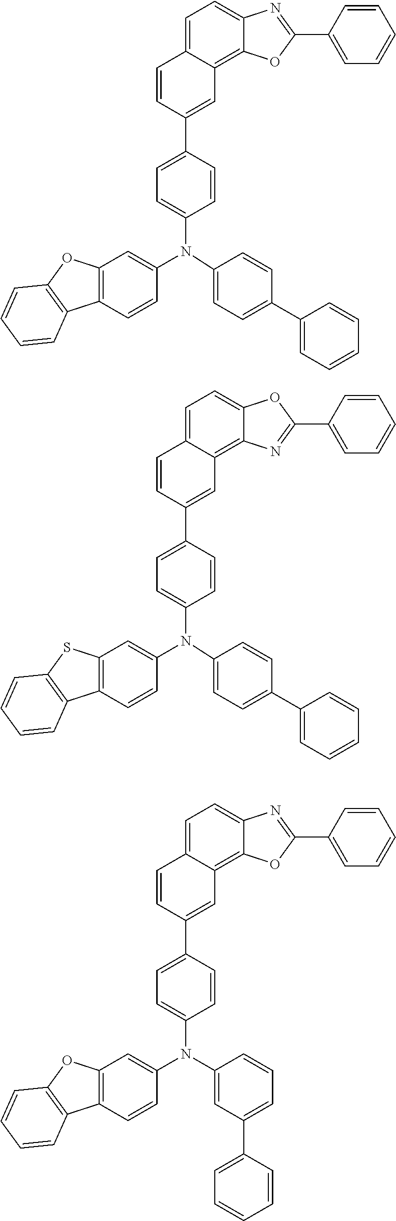

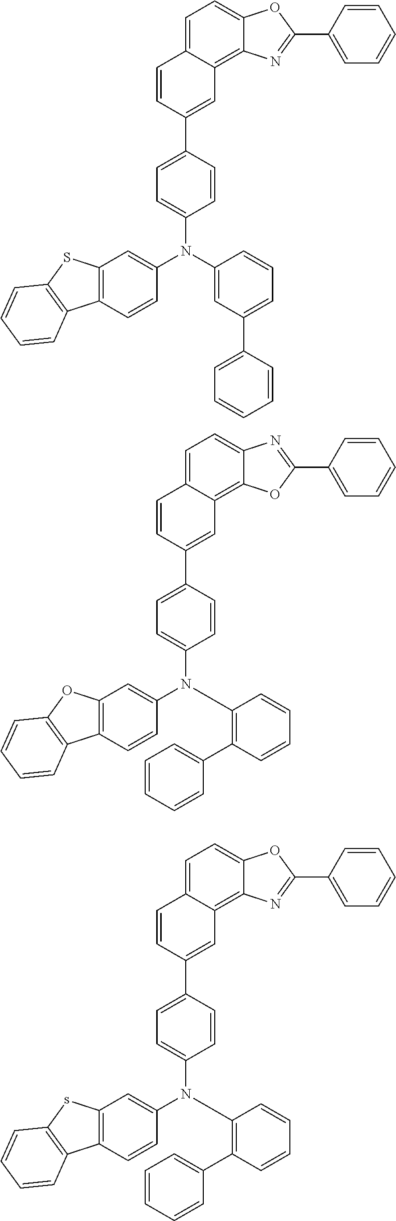

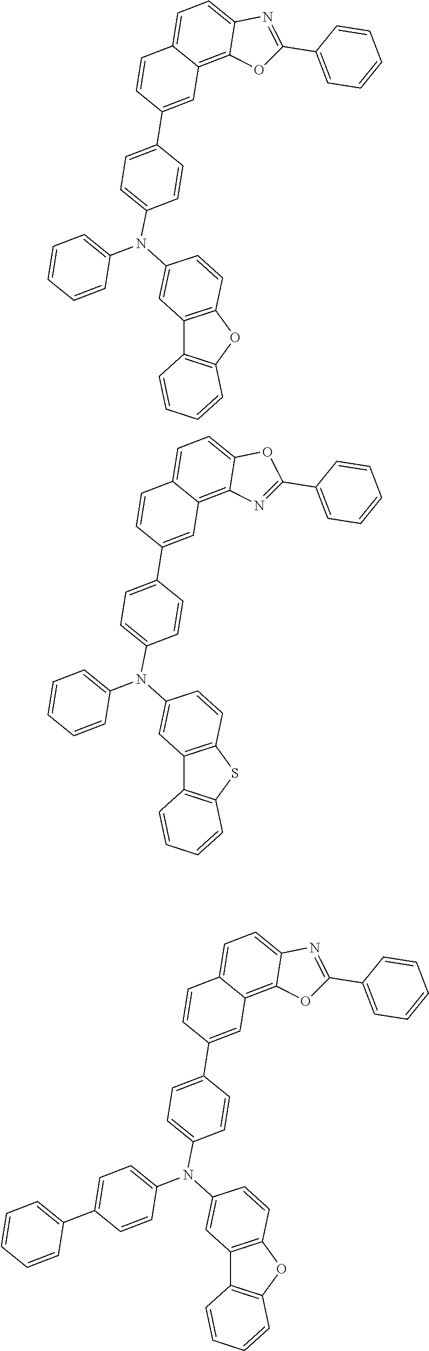

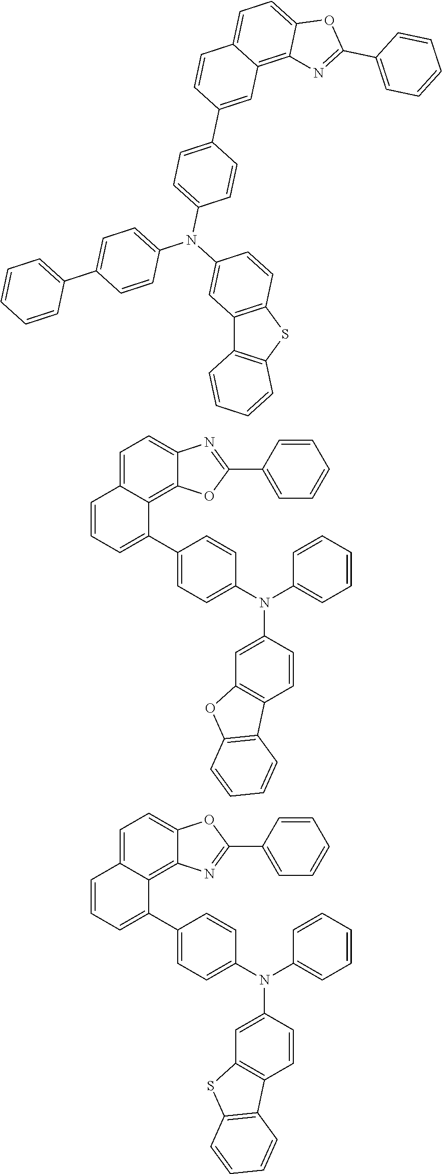

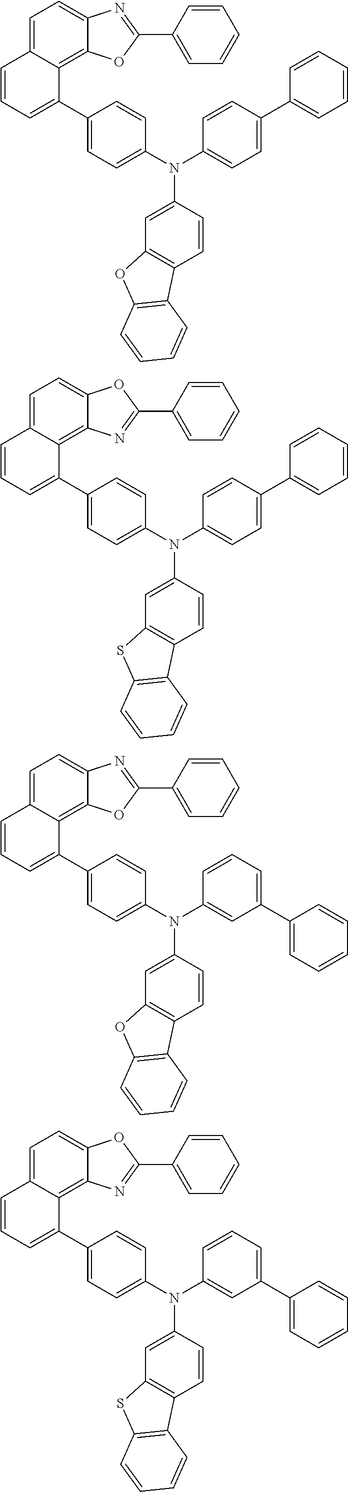

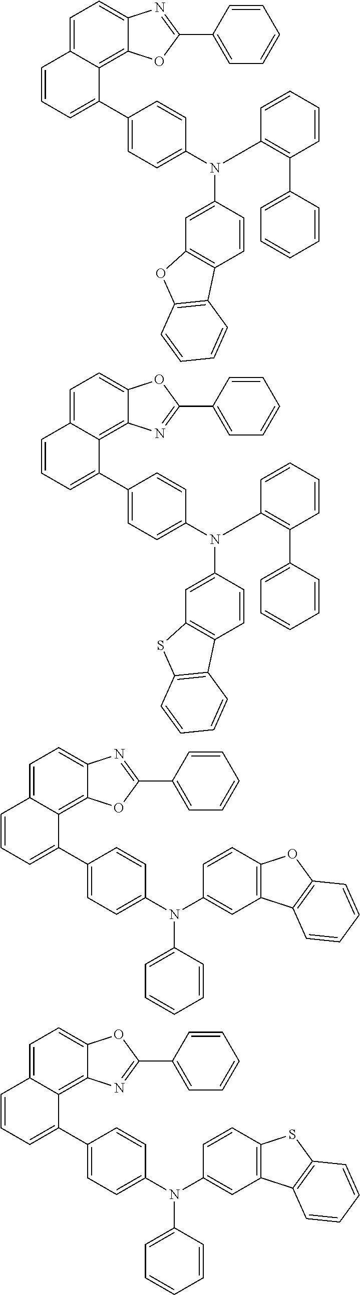

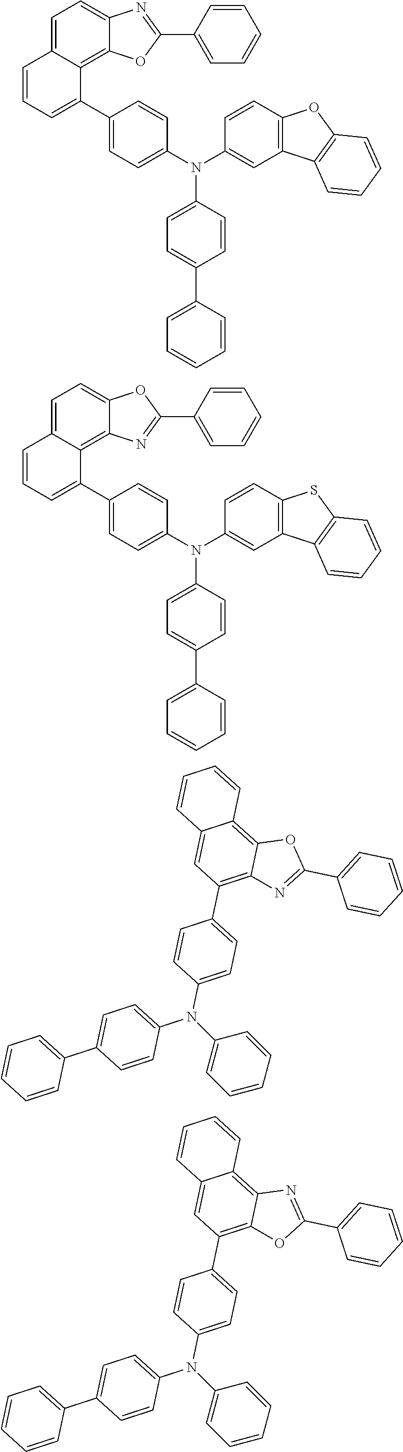

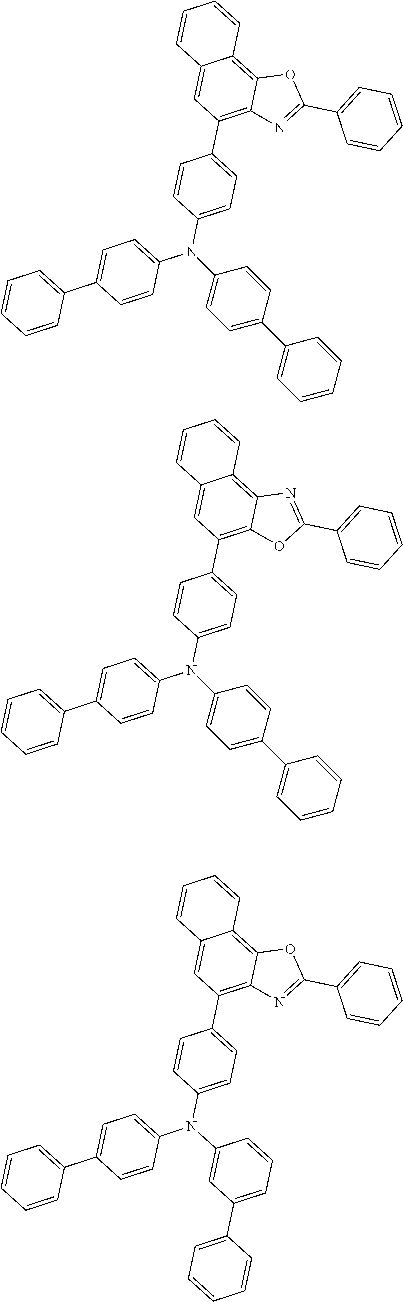 [SMILES: c1ccc(-c2ccc(N(c3ccc(-c4ccccc4)cc3)c3ccc(-c4cc5ccccc5c5oc(-c6ccccc6)nc45)cc3)cc2)cc1 ; c1ccc(-c2ccc(N(c3ccc(-c4ccccc4)cc3)c3ccc(-c4cc5ccccc5c5nc(-c6ccccc6)oc45)cc3)cc2)cc1 ; c1ccc(-c2ccc(N(c3ccc(-c4cc5ccccc5c5oc(-c6ccccc6)nc45)cc3)c3cccc(-c4ccccc4)c3)cc2)cc1]

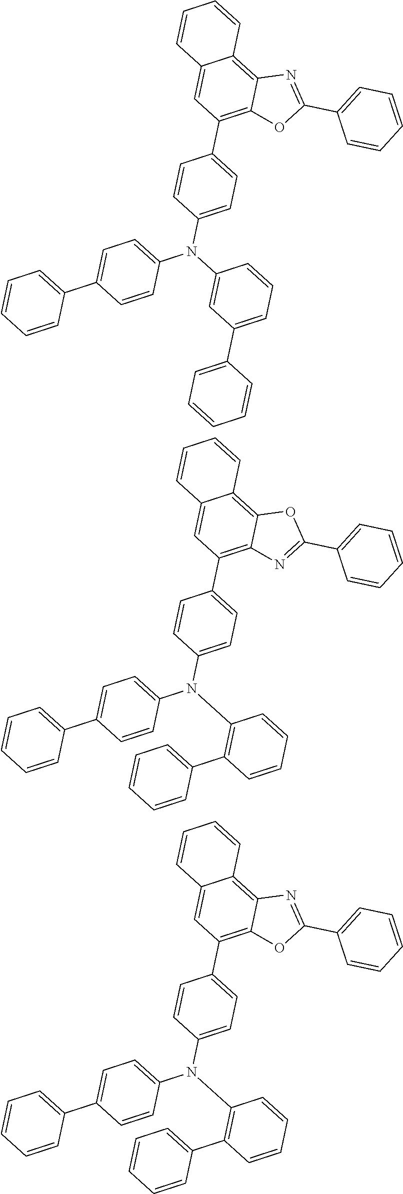

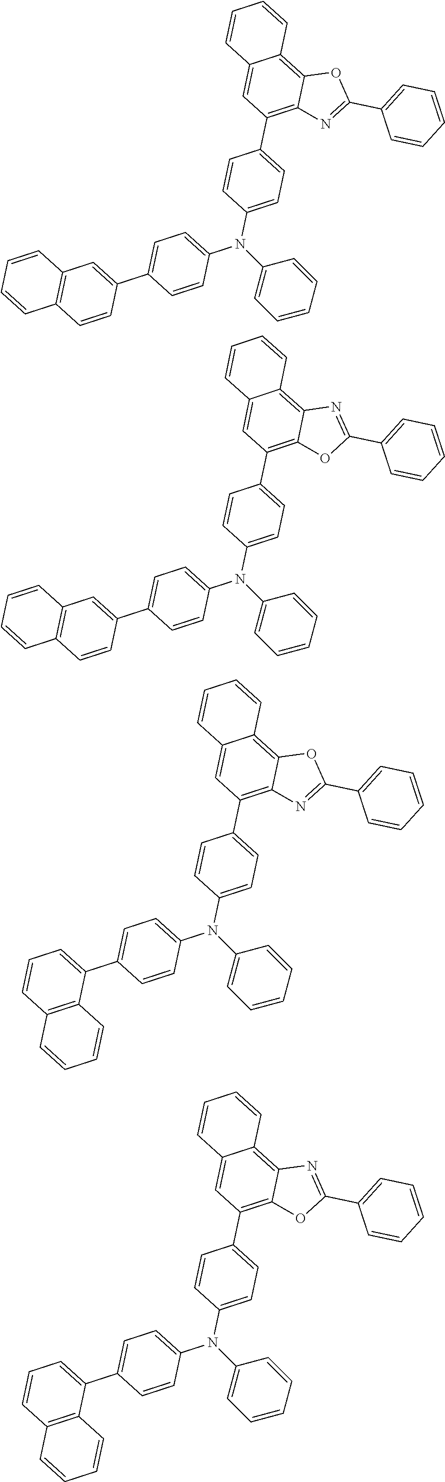

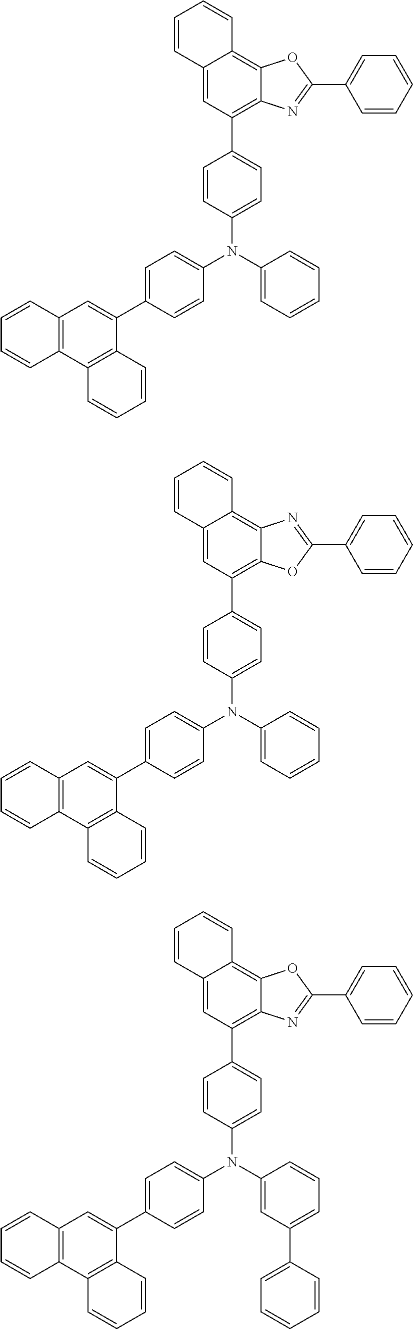 [SMILES: c1ccc(-c2nc3c(-c4ccc(N(c5ccccc5)c5ccc(-c6cc7ccccc7c7ccccc67)cc5)cc4)cc4ccccc4c3o2)cc1 ; c1ccc(-c2nc3c(o2)c(-c2ccc(N(c4ccccc4)c4ccc(-c5cc6ccccc6c6ccccc56)cc4)cc2)cc2ccccc23)cc1 ; c1ccc(-c2cccc(N(c3ccc(-c4cc5ccccc5c5ccccc45)cc3)c3ccc(-c4cc5ccccc5c5oc(-c6ccccc6)nc45)cc3)c2)cc1]

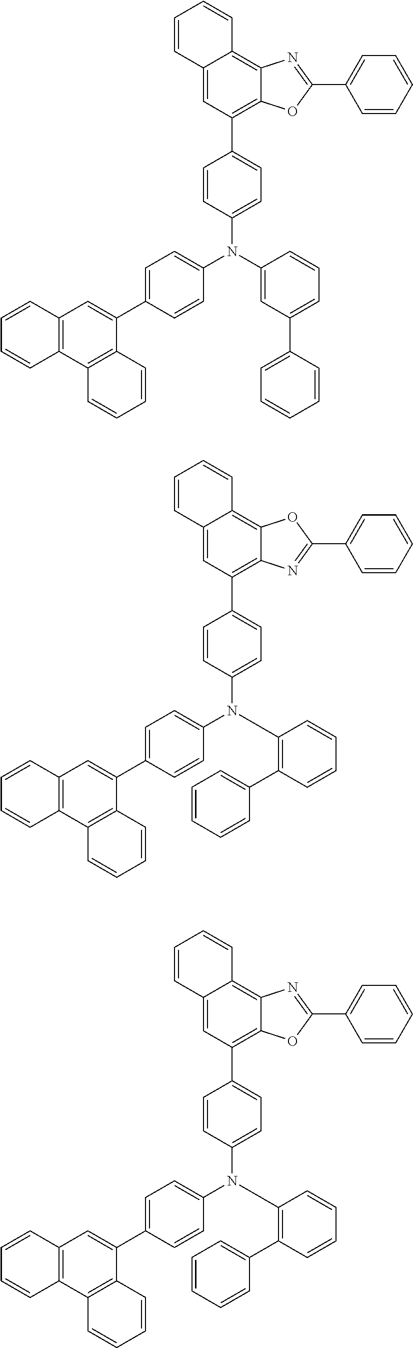 [SMILES: c1ccc(-c2cccc(N(c3ccc(-c4cc5ccccc5c5ccccc45)cc3)c3ccc(-c4cc5ccccc5c5nc(-c6ccccc6)oc45)cc3)c2)cc1 ; c1ccc(-c2nc3c(-c4ccc(N(c5ccc(-c6cc7ccccc7c7ccccc67)cc5)c5ccccc5-c5ccccc5)cc4)cc4ccccc4c3o2)cc1 ; c1ccc(-c2nc3c(o2)c(-c2ccc(N(c4ccc(-c5cc6ccccc6c6ccccc56)cc4)c4ccccc4-c4ccccc4)cc2)cc2ccccc23)cc1]

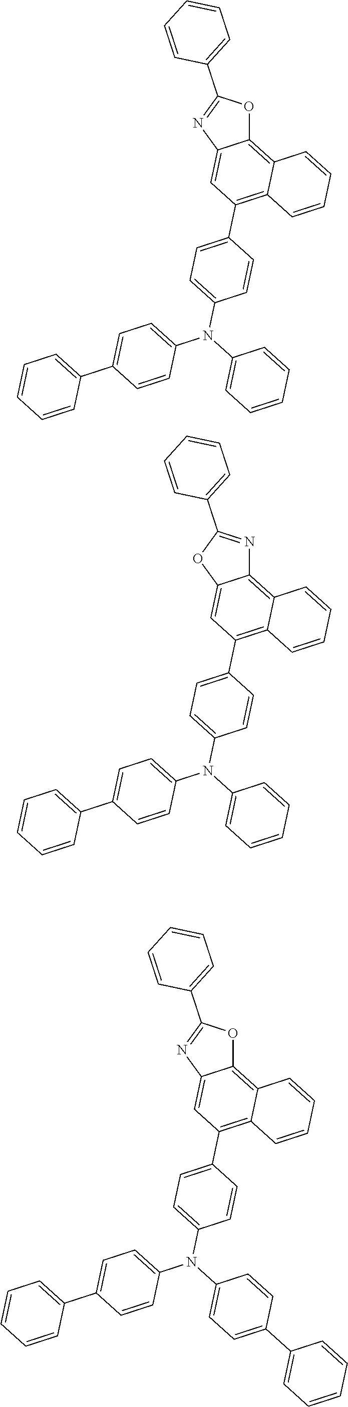

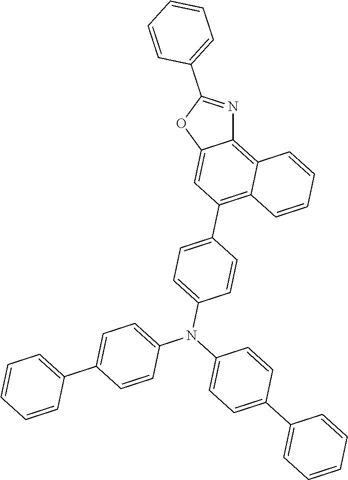 [SMILES: c1ccc(-c2ccc(N(c3ccc(-c4ccccc4)cc3)c3ccc(-c4cc5oc(-c6ccccc6)nc5c5ccccc45)cc3)cc2)cc1]

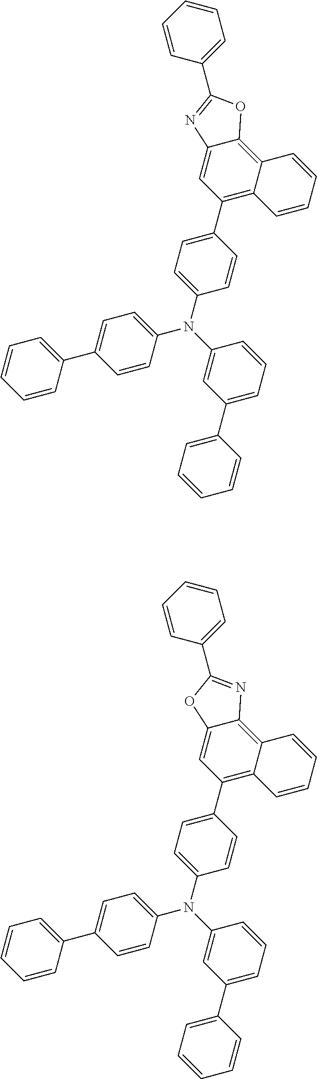 [SMILES: c1ccc(-c2ccc(N(c3ccc(-c4cc5nc(-c6ccccc6)oc5c5ccccc45)cc3)c3cccc(-c4ccccc4)c3)cc2)cc1 ; c1ccc(-c2ccc(N(c3ccc(-c4cc5oc(-c6ccccc6)nc5c5ccccc45)cc3)c3cccc(-c4ccccc4)c3)cc2)cc1]

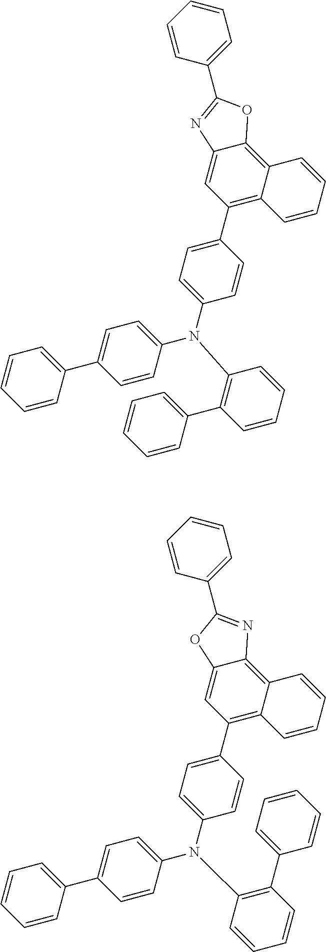 [SMILES: c1ccc(-c2ccc(N(c3ccc(-c4cc5nc(-c6ccccc6)oc5c5ccccc45)cc3)c3ccccc3-c3ccccc3)cc2)cc1 ; c1ccc(-c2ccc(N(c3ccc(-c4cc5oc(-c6ccccc6)nc5c5ccccc45)cc3)c3ccccc3-c3ccccc3)cc2)cc1]

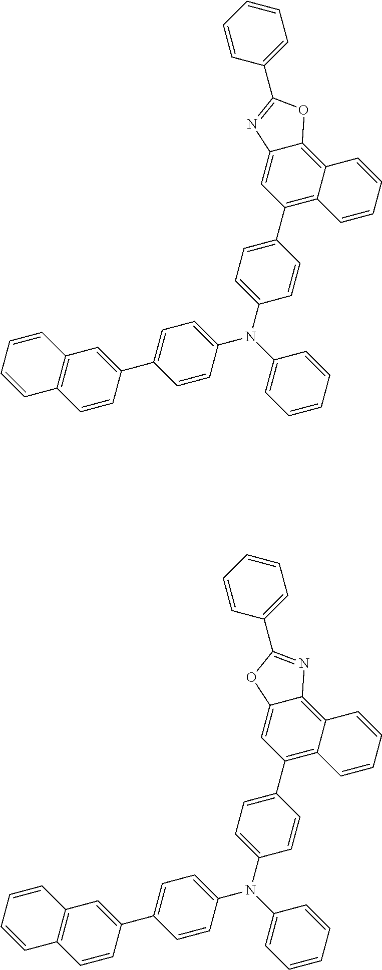 [SMILES: c1ccc(-c2nc3cc(-c4ccc(N(c5ccccc5)c5ccc(-c6ccc7ccccc7c6)cc5)cc4)c4ccccc4c3o2)cc1 ; c1ccc(-c2nc3c(cc(-c4ccc(N(c5ccccc5)c5ccc(-c6ccc7ccccc7c6)cc5)cc4)c4ccccc43)o2)cc1]

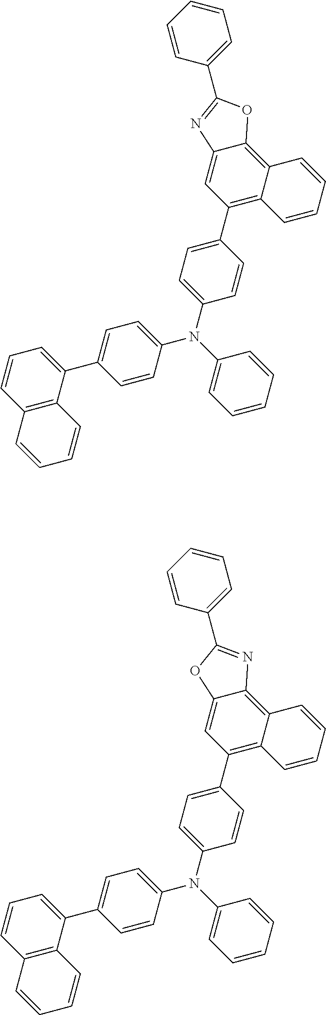 [SMILES: c1ccc(-c2nc3cc(-c4ccc(N(c5ccccc5)c5ccc(-c6cccc7ccccc67)cc5)cc4)c4ccccc4c3o2)cc1 ; c1ccc(-c2nc3c(cc(-c4ccc(N(c5ccccc5)c5ccc(-c6cccc7ccccc67)cc5)cc4)c4ccccc43)o2)cc1]

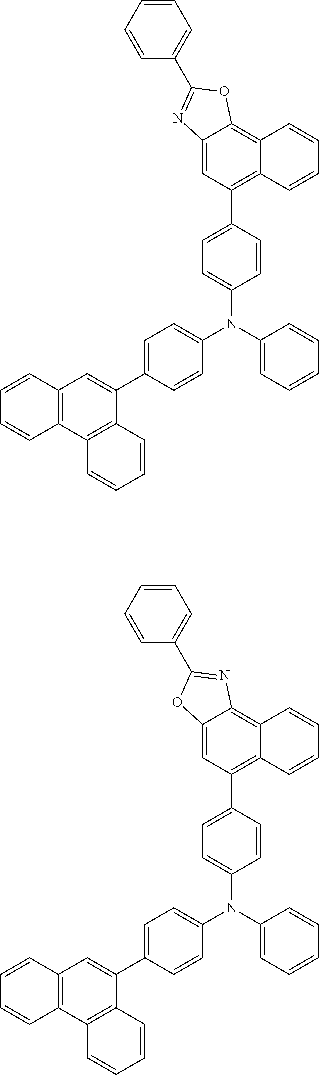 [SMILES: c1ccc(-c2nc3cc(-c4ccc(N(c5ccccc5)c5ccc(-c6cc7ccccc7c7ccccc67)cc5)cc4)c4ccccc4c3o2)cc1 ; c1ccc(-c2nc3c(cc(-c4ccc(N(c5ccccc5)c5ccc(-c6cc7ccccc7c7ccccc67)cc5)cc4)c4ccccc43)o2)cc1]

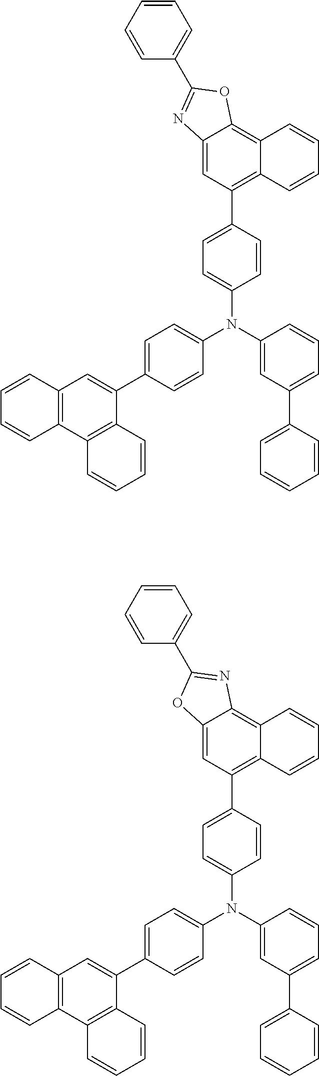 [SMILES: c1ccc(-c2cccc(N(c3ccc(-c4cc5ccccc5c5ccccc45)cc3)c3ccc(-c4cc5nc(-c6ccccc6)oc5c5ccccc45)cc3)c2)cc1 ; c1ccc(-c2cccc(N(c3ccc(-c4cc5ccccc5c5ccccc45)cc3)c3ccc(-c4cc5oc(-c6ccccc6)nc5c5ccccc45)cc3)c2)cc1]

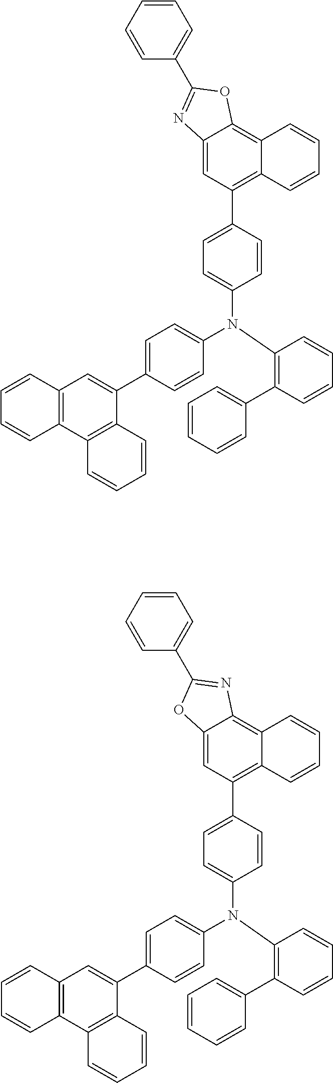 [SMILES: c1ccc(-c2nc3cc(-c4ccc(N(c5ccc(-c6cc7ccccc7c7ccccc67)cc5)c5ccccc5-c5ccccc5)cc4)c4ccccc4c3o2)cc1 ; c1ccc(-c2nc3c(cc(-c4ccc(N(c5ccc(-c6cc7ccccc7c7ccccc67)cc5)c5ccccc5-c5ccccc5)cc4)c4ccccc43)o2)cc1]

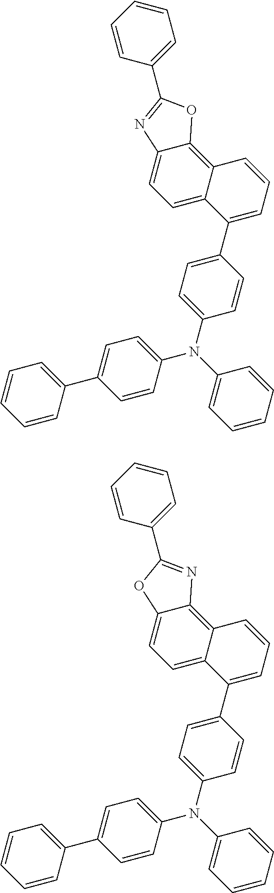 [SMILES: c1ccc(-c2ccc(N(c3ccccc3)c3ccc(-c4cccc5c4ccc4nc(-c6ccccc6)oc45)cc3)cc2)cc1 ; c1ccc(-c2ccc(N(c3ccccc3)c3ccc(-c4cccc5c4ccc4oc(-c6ccccc6)nc45)cc3)cc2)cc1]

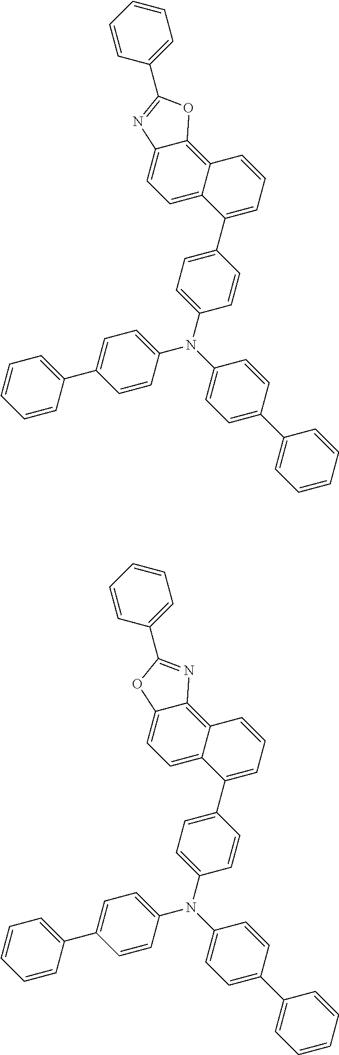 [SMILES: c1ccc(-c2ccc(N(c3ccc(-c4ccccc4)cc3)c3ccc(-c4cccc5c4ccc4nc(-c6ccccc6)oc45)cc3)cc2)cc1 ; c1ccc(-c2ccc(N(c3ccc(-c4ccccc4)cc3)c3ccc(-c4cccc5c4ccc4oc(-c6ccccc6)nc45)cc3)cc2)cc1]

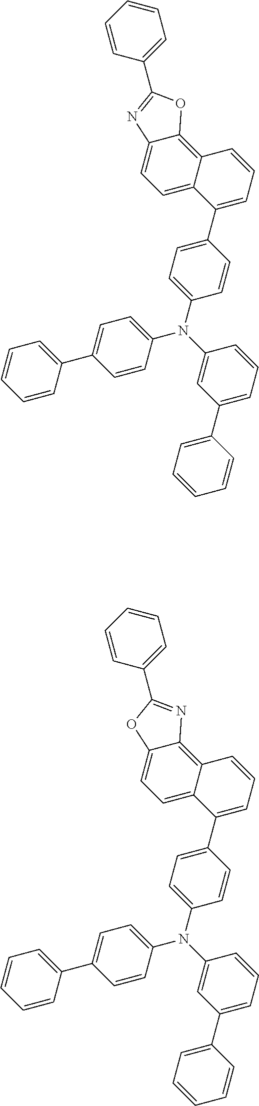 [SMILES: c1ccc(-c2ccc(N(c3ccc(-c4cccc5c4ccc4nc(-c6ccccc6)oc45)cc3)c3cccc(-c4ccccc4)c3)cc2)cc1 ; c1ccc(-c2ccc(N(c3ccc(-c4cccc5c4ccc4oc(-c6ccccc6)nc45)cc3)c3cccc(-c4ccccc4)c3)cc2)cc1]

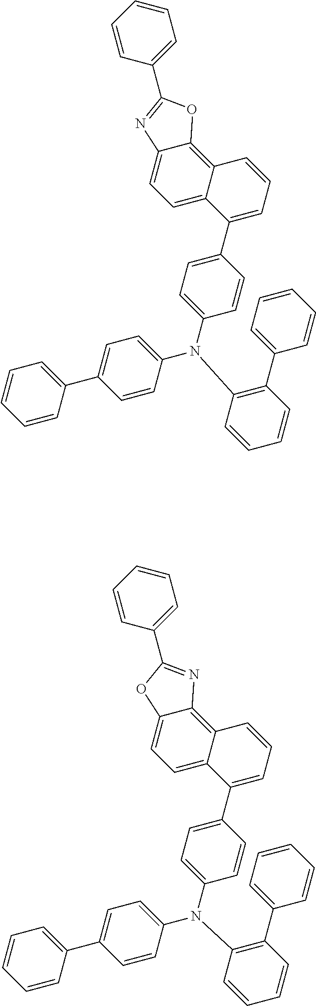 [SMILES: c1ccc(-c2ccc(N(c3ccc(-c4cccc5c4ccc4nc(-c6ccccc6)oc45)cc3)c3ccccc3-c3ccccc3)cc2)cc1 ; c1ccc(-c2ccc(N(c3ccc(-c4cccc5c4ccc4oc(-c6ccccc6)nc45)cc3)c3ccccc3-c3ccccc3)cc2)cc1]

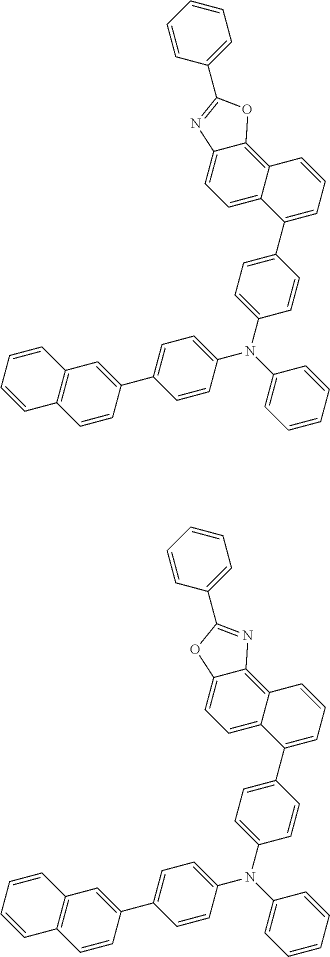 [SMILES: c1ccc(-c2nc3ccc4c(-c5ccc(N(c6ccccc6)c6ccc(-c7ccc8ccccc8c7)cc6)cc5)cccc4c3o2)cc1 ; c1ccc(-c2nc3c(ccc4c(-c5ccc(N(c6ccccc6)c6ccc(-c7ccc8ccccc8c7)cc6)cc5)cccc43)o2)cc1]

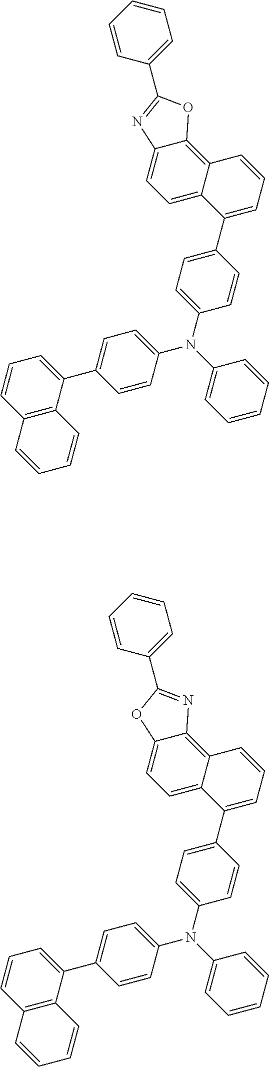 [SMILES: c1ccc(-c2nc3ccc4c(-c5ccc(N(c6ccccc6)c6ccc(-c7cccc8ccccc78)cc6)cc5)cccc4c3o2)cc1 ; c1ccc(-c2nc3c(ccc4c(-c5ccc(N(c6ccccc6)c6ccc(-c7cccc8ccccc78)cc6)cc5)cccc43)o2)cc1]

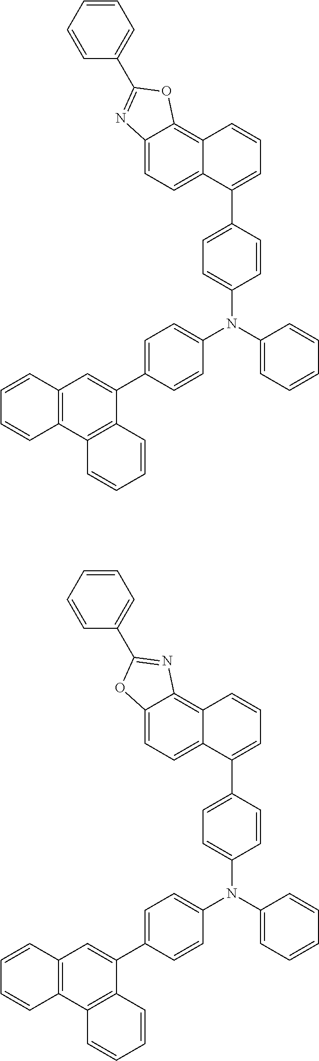 [SMILES: c1ccc(-c2nc3ccc4c(-c5ccc(N(c6ccccc6)c6ccc(-c7cc8ccccc8c8ccccc78)cc6)cc5)cccc4c3o2)cc1 ; c1ccc(-c2nc3c(ccc4c(-c5ccc(N(c6ccccc6)c6ccc(-c7cc8ccccc8c8ccccc78)cc6)cc5)cccc43)o2)cc1]

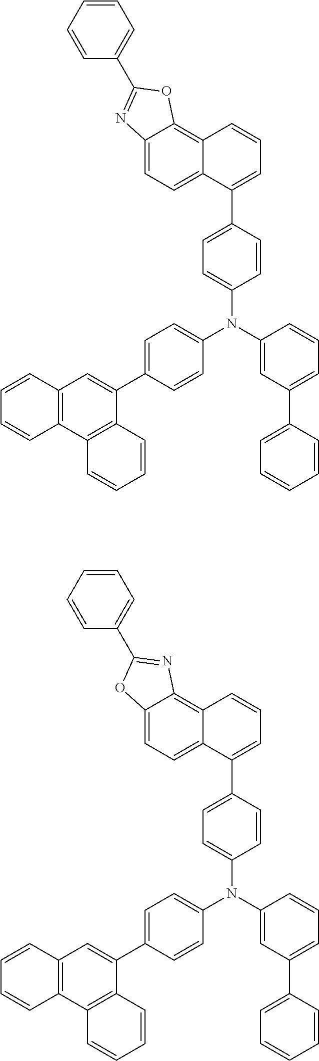 [SMILES: c1ccc(-c2cccc(N(c3ccc(-c4cc5ccccc5c5ccccc45)cc3)c3ccc(-c4cccc5c4ccc4nc(-c6ccccc6)oc45)cc3)c2)cc1 ; c1ccc(-c2cccc(N(c3ccc(-c4cc5ccccc5c5ccccc45)cc3)c3ccc(-c4cccc5c4ccc4oc(-c6ccccc6)nc45)cc3)c2)cc1]

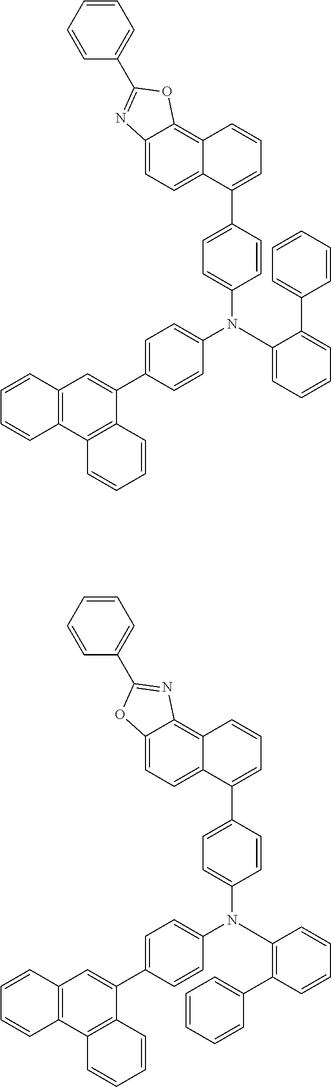 [SMILES: c1ccc(-c2nc3ccc4c(-c5ccc(N(c6ccc(-c7cc8ccccc8c8ccccc78)cc6)c6ccccc6-c6ccccc6)cc5)cccc4c3o2)cc1 ; c1ccc(-c2nc3c(ccc4c(-c5ccc(N(c6ccc(-c7cc8ccccc8c8ccccc78)cc6)c6ccccc6-c6ccccc6)cc5)cccc43)o2)cc1]

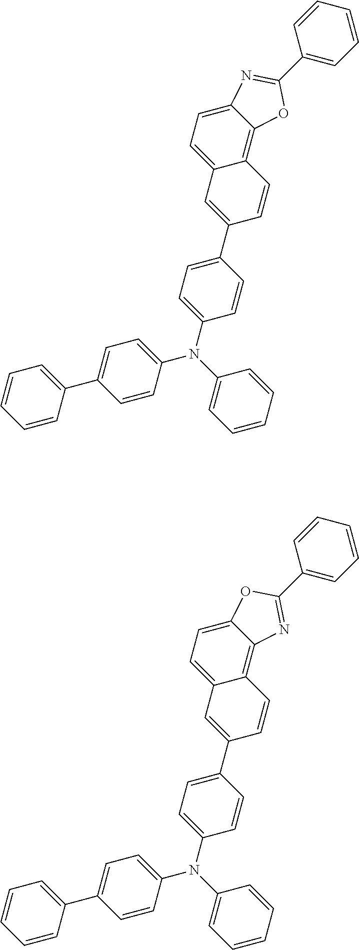 [SMILES: c1ccc(-c2ccc(N(c3ccccc3)c3ccc(-c4ccc5c(ccc6nc(-c7ccccc7)oc65)c4)cc3)cc2)cc1 ; c1ccc(-c2ccc(N(c3ccccc3)c3ccc(-c4ccc5c(ccc6oc(-c7ccccc7)nc65)c4)cc3)cc2)cc1]

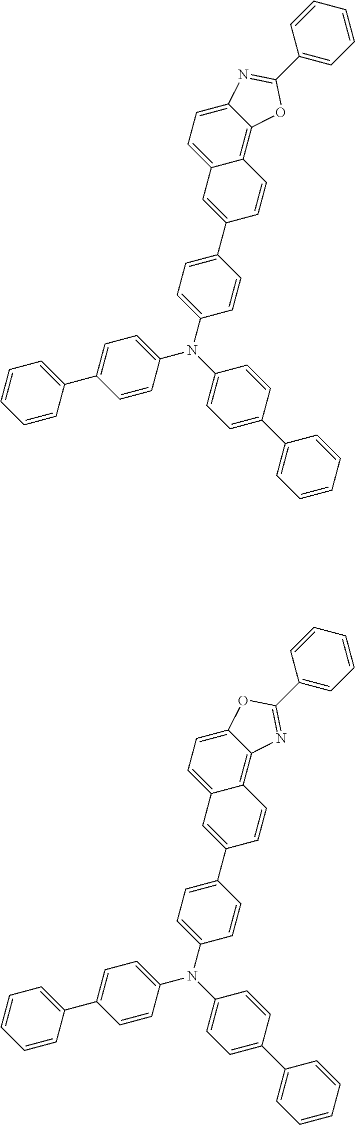 [SMILES: c1ccc(-c2ccc(N(c3ccc(-c4ccccc4)cc3)c3ccc(-c4ccc5c(ccc6nc(-c7ccccc7)oc65)c4)cc3)cc2)cc1 ; c1ccc(-c2ccc(N(c3ccc(-c4ccccc4)cc3)c3ccc(-c4ccc5c(ccc6oc(-c7ccccc7)nc65)c4)cc3)cc2)cc1]

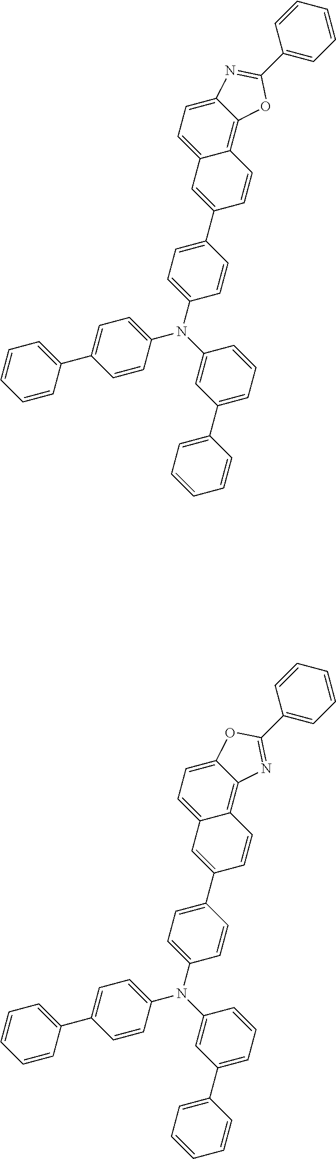 [SMILES: c1ccc(-c2ccc(N(c3ccc(-c4ccc5c(ccc6nc(-c7ccccc7)oc65)c4)cc3)c3cccc(-c4ccccc4)c3)cc2)cc1 ; c1ccc(-c2ccc(N(c3ccc(-c4ccc5c(ccc6oc(-c7ccccc7)nc65)c4)cc3)c3cccc(-c4ccccc4)c3)cc2)cc1]

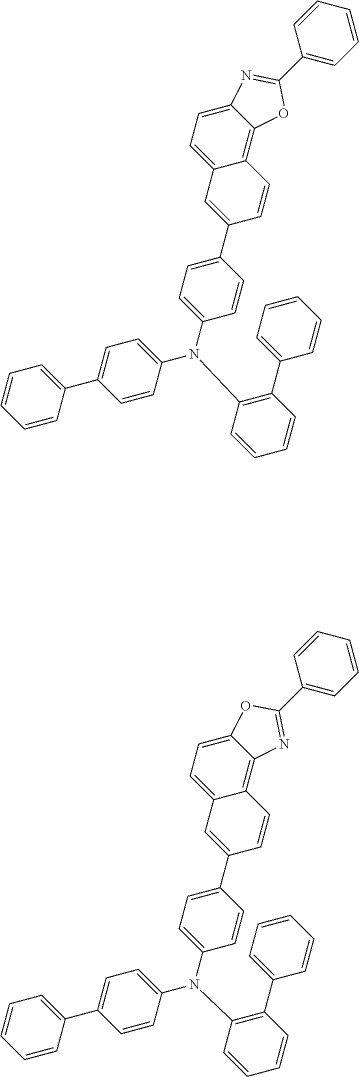 [SMILES: c1ccc(-c2ccc(N(c3ccc(-c4ccc5c(ccc6nc(-c7ccccc7)oc65)c4)cc3)c3ccccc3-c3ccccc3)cc2)cc1 ; c1ccc(-c2ccc(N(c3ccc(-c4ccc5c(ccc6oc(-c7ccccc7)nc65)c4)cc3)c3ccccc3-c3ccccc3)cc2)cc1]

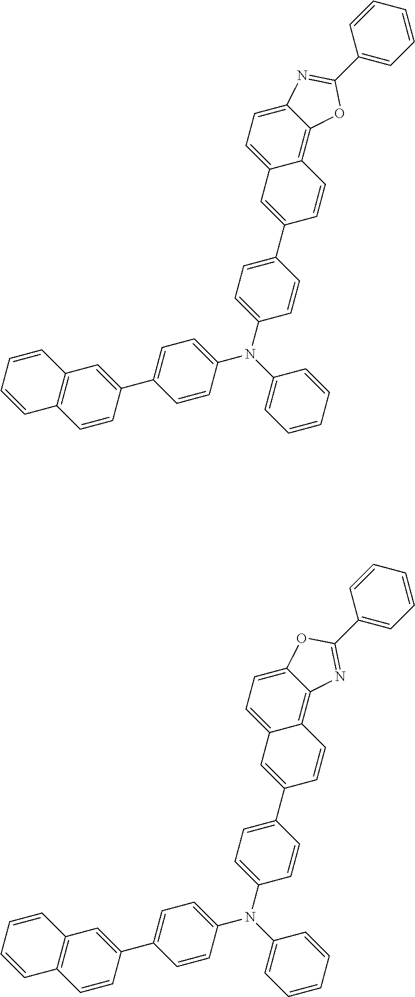 [SMILES: c1ccc(-c2nc3ccc4cc(-c5ccc(N(c6ccccc6)c6ccc(-c7ccc8ccccc8c7)cc6)cc5)ccc4c3o2)cc1 ; c1ccc(-c2nc3c(ccc4cc(-c5ccc(N(c6ccccc6)c6ccc(-c7ccc8ccccc8c7)cc6)cc5)ccc43)o2)cc1]

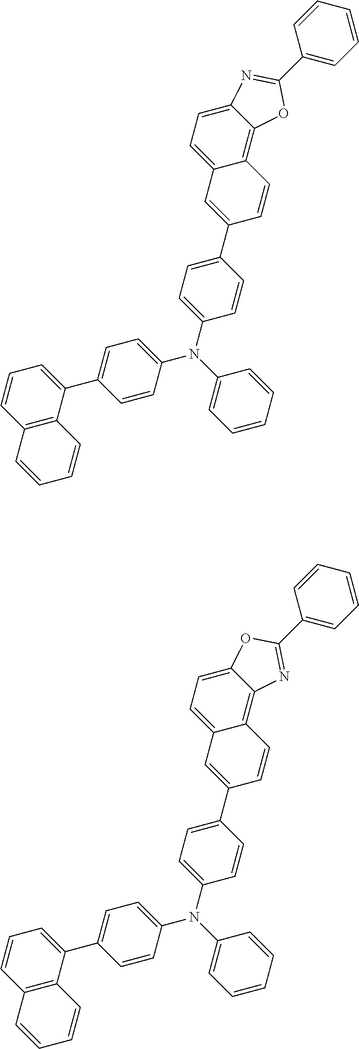 [SMILES: c1ccc(-c2nc3ccc4cc(-c5ccc(N(c6ccccc6)c6ccc(-c7cccc8ccccc78)cc6)cc5)ccc4c3o2)cc1 ; c1ccc(-c2nc3c(ccc4cc(-c5ccc(N(c6ccccc6)c6ccc(-c7cccc8ccccc78)cc6)cc5)ccc43)o2)cc1]

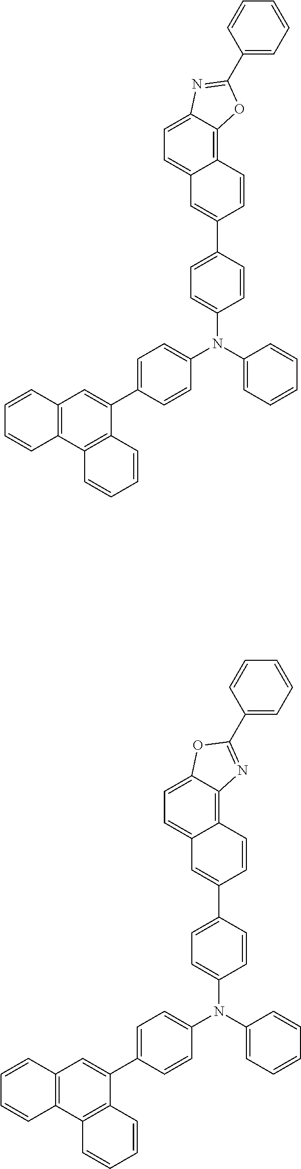 [SMILES: c1ccc(-c2nc3ccc4cc(-c5ccc(N(c6ccccc6)c6ccc(-c7cc8ccccc8c8ccccc78)cc6)cc5)ccc4c3o2)cc1 ; c1ccc(-c2nc3c(ccc4cc(-c5ccc(N(c6ccccc6)c6ccc(-c7cc8ccccc8c8ccccc78)cc6)cc5)ccc43)o2)cc1]

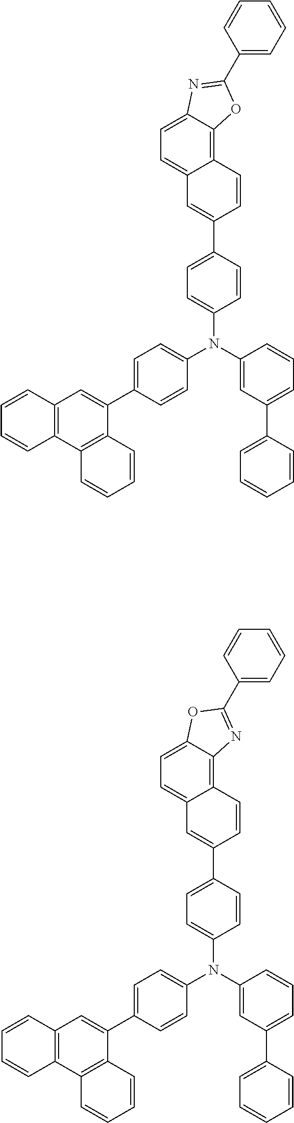 [SMILES: c1ccc(-c2cccc(N(c3ccc(-c4ccc5c(ccc6nc(-c7ccccc7)oc65)c4)cc3)c3ccc(-c4cc5ccccc5c5ccccc45)cc3)c2)cc1 ; c1ccc(-c2cccc(N(c3ccc(-c4ccc5c(ccc6oc(-c7ccccc7)nc65)c4)cc3)c3ccc(-c4cc5ccccc5c5ccccc45)cc3)c2)cc1]

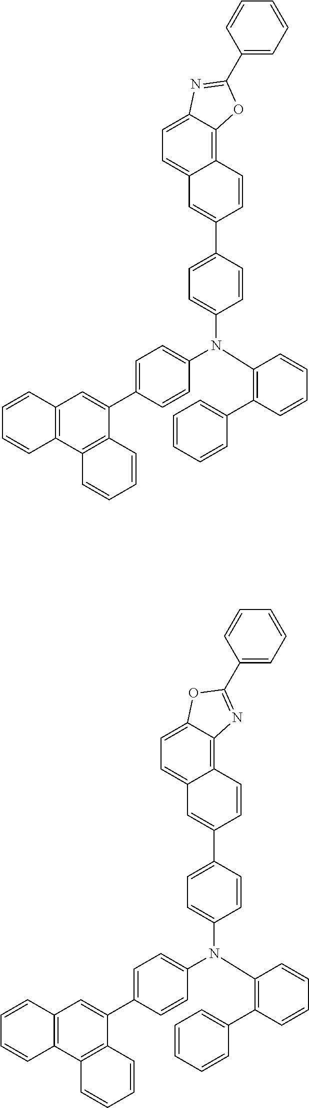 [SMILES: c1ccc(-c2nc3ccc4cc(-c5ccc(N(c6ccc(-c7cc8ccccc8c8ccccc78)cc6)c6ccccc6-c6ccccc6)cc5)ccc4c3o2)cc1 ; c1ccc(-c2nc3c(ccc4cc(-c5ccc(N(c6ccc(-c7cc8ccccc8c8ccccc78)cc6)c6ccccc6-c6ccccc6)cc5)ccc43)o2)cc1]

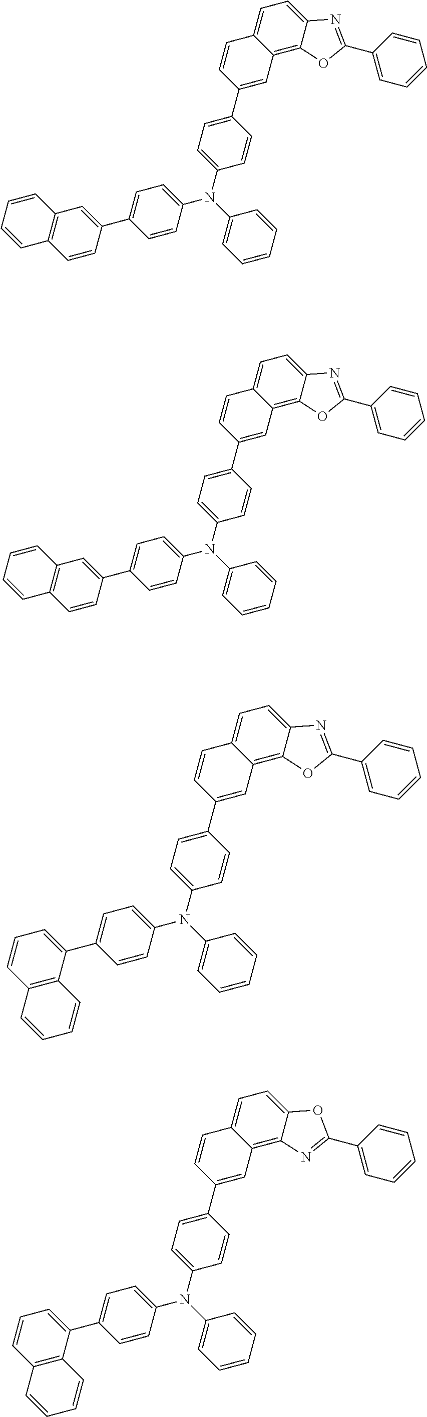 [SMILES: c1ccc(-c2nc3ccc4ccc(-c5ccc(N(c6ccccc6)c6ccc(-c7ccc8ccccc8c7)cc6)cc5)cc4c3o2)cc1 ; c1ccc(-c2nc3ccc4ccc(-c5ccc(N(c6ccccc6)c6ccc(-c7ccc8ccccc8c7)cc6)cc5)cc4c3o2)cc1 ; c1ccc(-c2nc3ccc4ccc(-c5ccc(N(c6ccccc6)c6ccc(-c7cccc8ccccc78)cc6)cc5)cc4c3o2)cc1 ; c1ccc(-c2nc3c(ccc4ccc(-c5ccc(N(c6ccccc6)c6ccc(-c7cccc8ccccc78)cc6)cc5)cc43)o2)cc1]

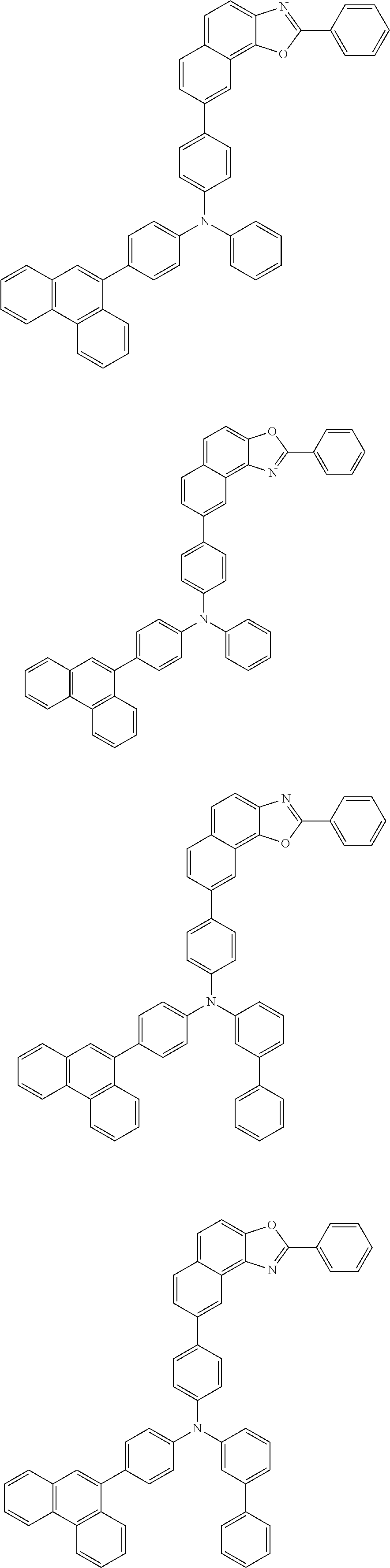 [SMILES: c1ccc(-c2nc3ccc4ccc(-c5ccc(N(c6ccccc6)c6ccc(-c7cc8ccccc8c8ccccc78)cc6)cc5)cc4c3o2)cc1 ; c1ccc(-c2nc3c(ccc4ccc(-c5ccc(N(c6ccccc6)c6ccc(-c7cc8ccccc8c8ccccc78)cc6)cc5)cc43)o2)cc1 ; c1ccc(-c2cccc(N(c3ccc(-c4ccc5ccc6nc(-c7ccccc7)oc6c5c4)cc3)c3ccc(-c4cc5ccccc5c5ccccc45)cc3)c2)cc1 ; c1ccc(-c2cccc(N(c3ccc(-c4ccc5ccc6oc(-c7ccccc7)nc6c5c4)cc3)c3ccc(-c4cc5ccccc5c5ccccc45)cc3)c2)cc1]

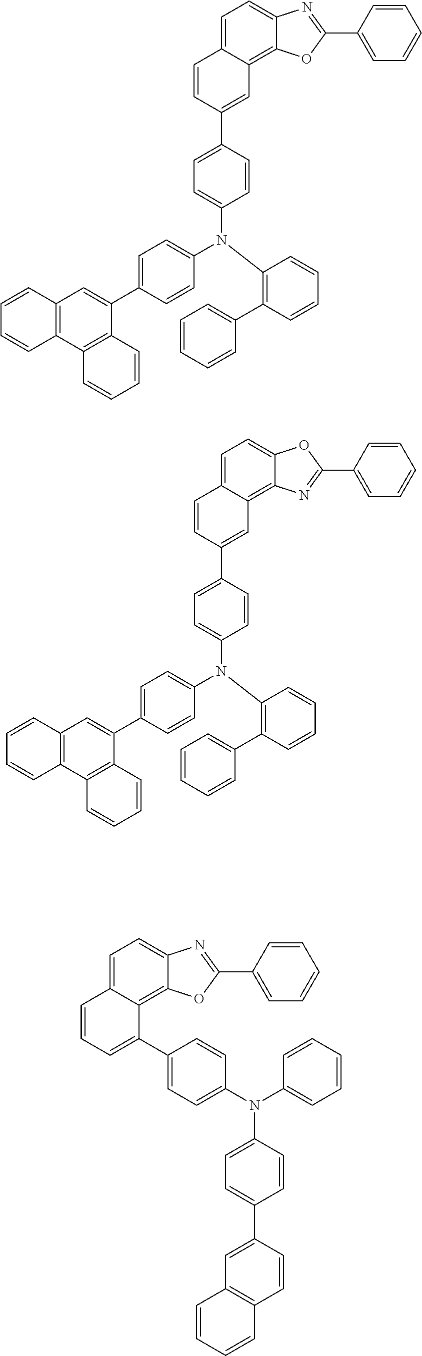 [SMILES: c1ccc(-c2nc3ccc4ccc(-c5ccc(N(c6ccc(-c7cc8ccccc8c8ccccc78)cc6)c6ccccc6-c6ccccc6)cc5)cc4c3o2)cc1 ; c1ccc(-c2nc3c(ccc4ccc(-c5ccc(N(c6ccc(-c7cc8ccccc8c8ccccc78)cc6)c6ccccc6-c6ccccc6)cc5)cc43)o2)cc1 ; c1ccc(-c2nc3ccc4cccc(-c5ccc(N(c6ccccc6)c6ccc(-c7ccc8ccccc8c7)cc6)cc5)c4c3o2)cc1]

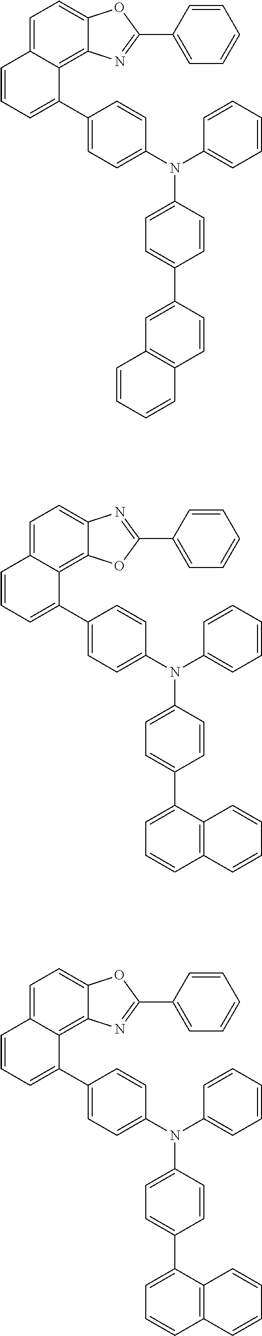 [SMILES: c1ccc(-c2nc3c(ccc4cccc(-c5ccc(N(c6ccccc6)c6ccc(-c7ccc8ccccc8c7)cc6)cc5)c43)o2)cc1 ; c1ccc(-c2nc3ccc4cccc(-c5ccc(N(c6ccccc6)c6ccc(-c7cccc8ccccc78)cc6)cc5)c4c3o2)cc1 ; c1ccc(-c2nc3c(ccc4cccc(-c5ccc(N(c6ccccc6)c6ccc(-c7cccc8ccccc78)cc6)cc5)c43)o2)cc1]

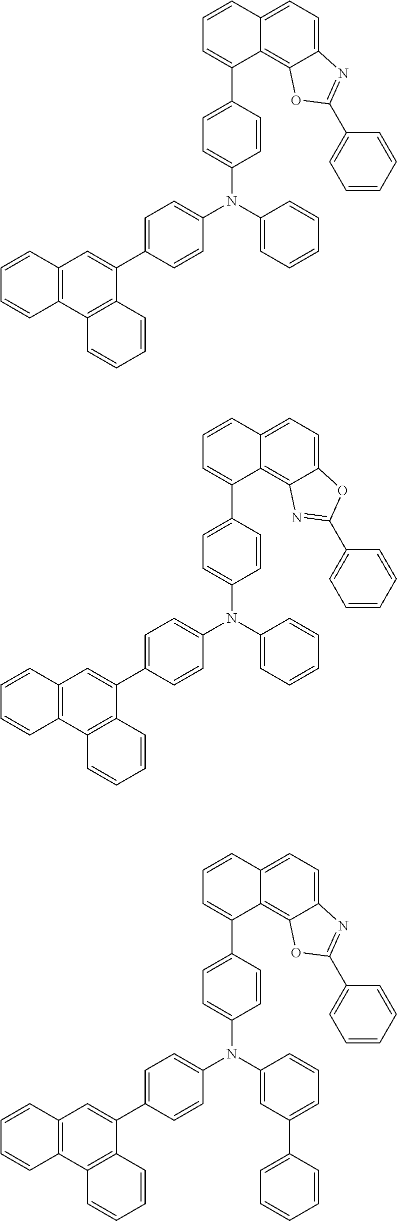 [SMILES: c1ccc(-c2nc3ccc4cccc(-c5ccc(N(c6ccccc6)c6ccc(-c7cc8ccccc8c8ccccc78)cc6)cc5)c4c3o2)cc1 ; c1ccc(-c2nc3c(ccc4cccc(-c5ccc(N(c6ccccc6)c6ccc(-c7cc8ccccc8c8ccccc78)cc6)cc5)c43)o2)cc1 ; c1ccc(-c2cccc(N(c3ccc(-c4cc5ccccc5c5ccccc45)cc3)c3ccc(-c4cccc5ccc6nc(-c7ccccc7)oc6c45)cc3)c2)cc1]

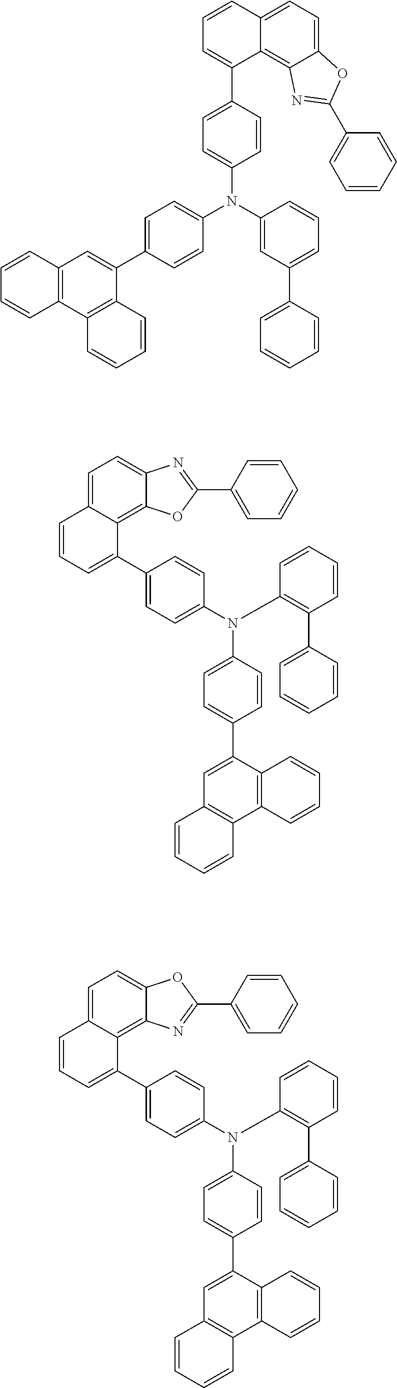 [SMILES: c1ccc(-c2cccc(N(c3ccc(-c4cc5ccccc5c5ccccc45)cc3)c3ccc(-c4cccc5ccc6oc(-c7ccccc7)nc6c45)cc3)c2)cc1 ; c1ccc(-c2nc3ccc4cccc(-c5ccc(N(c6ccc(-c7cc8ccccc8c8ccccc78)cc6)c6ccccc6-c6ccccc6)cc5)c4c3o2)cc1 ; c1ccc(-c2nc3c(ccc4cccc(-c5ccc(N(c6ccc(-c7cc8ccccc8c8ccccc78)cc6)c6ccccc6-c6ccccc6)cc5)c43)o2)cc1]

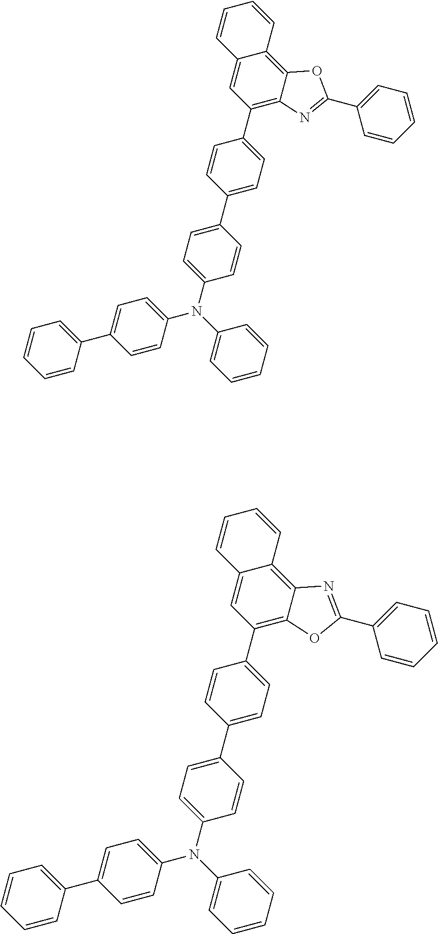 [SMILES: c1ccc(-c2ccc(N(c3ccccc3)c3ccc(-c4ccc(-c5cc6ccccc6c6oc(-c7ccccc7)nc56)cc4)cc3)cc2)cc1 ; c1ccc(-c2ccc(N(c3ccccc3)c3ccc(-c4ccc(-c5cc6ccccc6c6nc(-c7ccccc7)oc56)cc4)cc3)cc2)cc1]

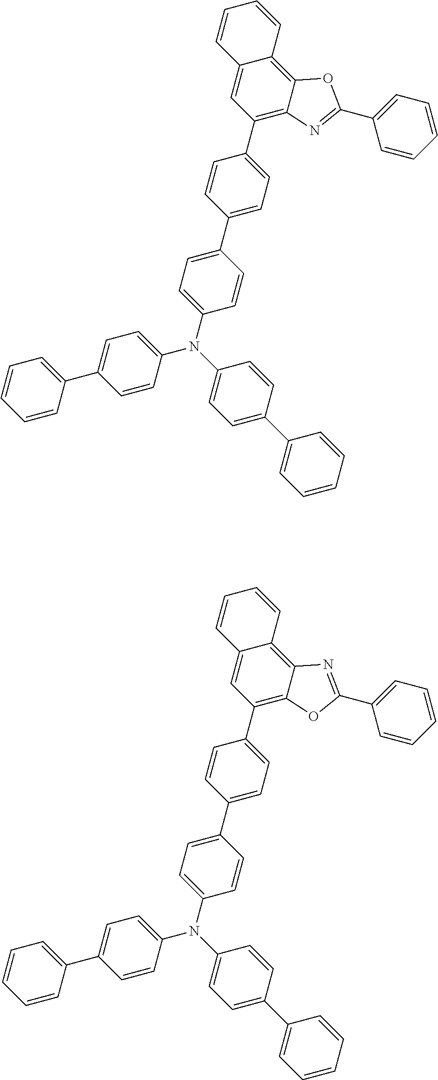 [SMILES: c1ccc(-c2ccc(N(c3ccc(-c4ccccc4)cc3)c3ccc(-c4ccc(-c5cc6ccccc6c6oc(-c7ccccc7)nc56)cc4)cc3)cc2)cc1 ; c1ccc(-c2ccc(N(c3ccc(-c4ccccc4)cc3)c3ccc(-c4ccc(-c5cc6ccccc6c6nc(-c7ccccc7)oc56)cc4)cc3)cc2)cc1]

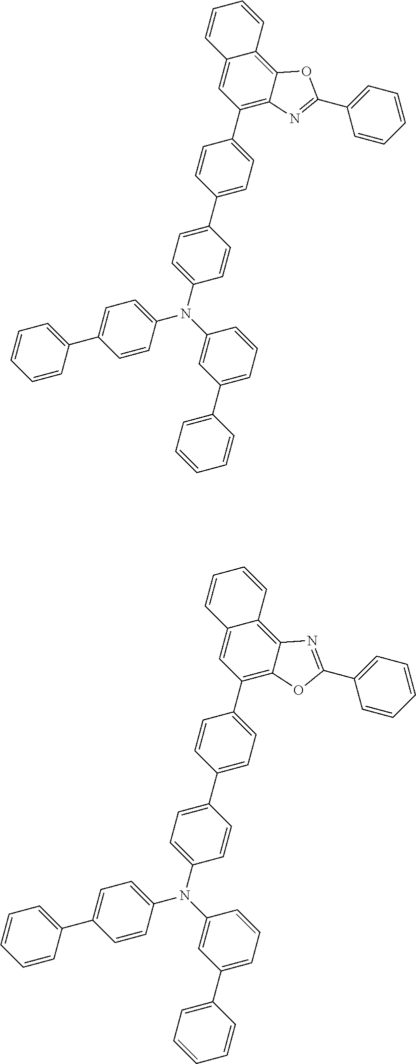 [SMILES: c1ccc(-c2ccc(N(c3ccc(-c4ccc(-c5cc6ccccc6c6oc(-c7ccccc7)nc56)cc4)cc3)c3cccc(-c4ccccc4)c3)cc2)cc1 ; c1ccc(-c2ccc(N(c3ccc(-c4ccc(-c5cc6ccccc6c6nc(-c7ccccc7)oc56)cc4)cc3)c3cccc(-c4ccccc4)c3)cc2)cc1]

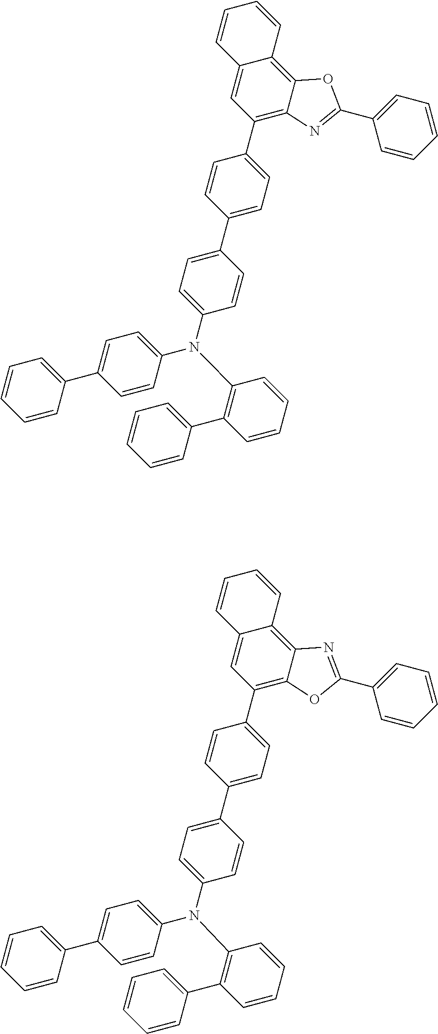 [SMILES: c1ccc(-c2ccc(N(c3ccc(-c4ccc(-c5cc6ccccc6c6oc(-c7ccccc7)nc56)cc4)cc3)c3ccccc3-c3ccccc3)cc2)cc1 ; c1ccc(-c2ccc(N(c3ccc(-c4ccc(-c5cc6ccccc6c6nc(-c7ccccc7)oc56)cc4)cc3)c3ccccc3-c3ccccc3)cc2)cc1]

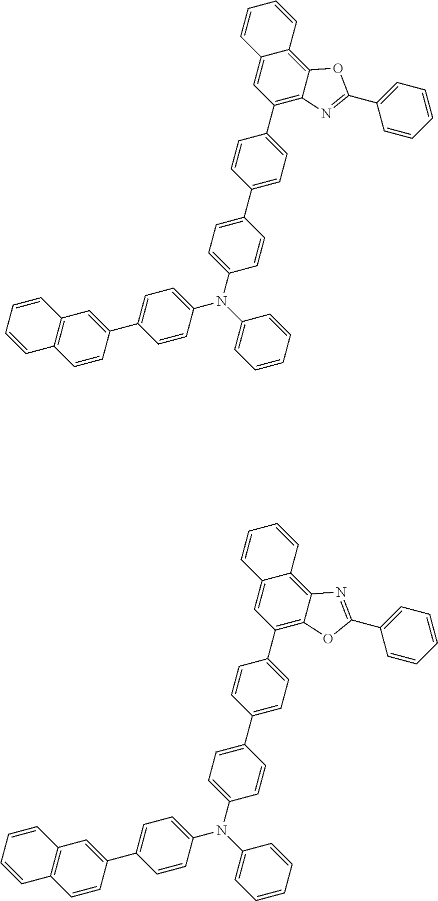 [SMILES: c1ccc(-c2nc3c(-c4ccc(-c5ccc(N(c6ccccc6)c6ccc(-c7ccc8ccccc8c7)cc6)cc5)cc4)cc4ccccc4c3o2)cc1 ; c1ccc(-c2nc3c(o2)c(-c2ccc(-c4ccc(N(c5ccccc5)c5ccc(-c6ccc7ccccc7c6)cc5)cc4)cc2)cc2ccccc23)cc1]

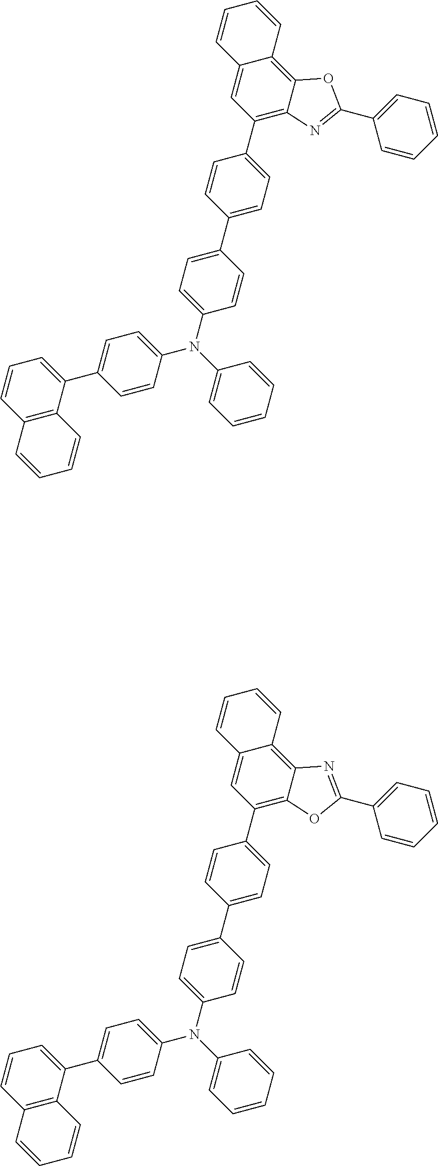 [SMILES: c1ccc(-c2nc3c(-c4ccc(-c5ccc(N(c6ccccc6)c6ccc(-c7cccc8ccccc78)cc6)cc5)cc4)cc4ccccc4c3o2)cc1 ; c1ccc(-c2nc3c(o2)c(-c2ccc(-c4ccc(N(c5ccccc5)c5ccc(-c6cccc7ccccc67)cc5)cc4)cc2)cc2ccccc23)cc1]

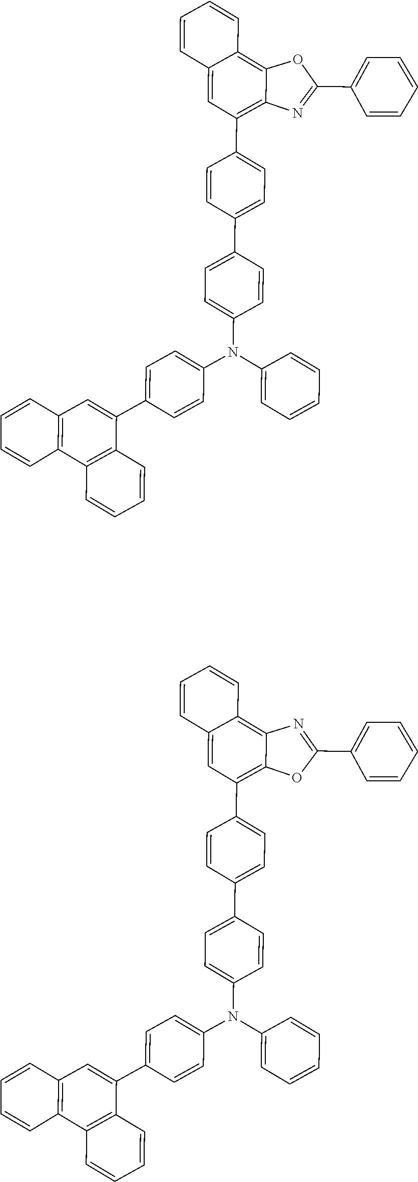 [SMILES: c1ccc(-c2nc3c(-c4ccc(-c5ccc(N(c6ccccc6)c6ccc(-c7cc8ccccc8c8ccccc78)cc6)cc5)cc4)cc4ccccc4c3o2)cc1 ; c1ccc(-c2nc3c(o2)c(-c2ccc(-c4ccc(N(c5ccccc5)c5ccc(-c6cc7ccccc7c7ccccc67)cc5)cc4)cc2)cc2ccccc23)cc1]

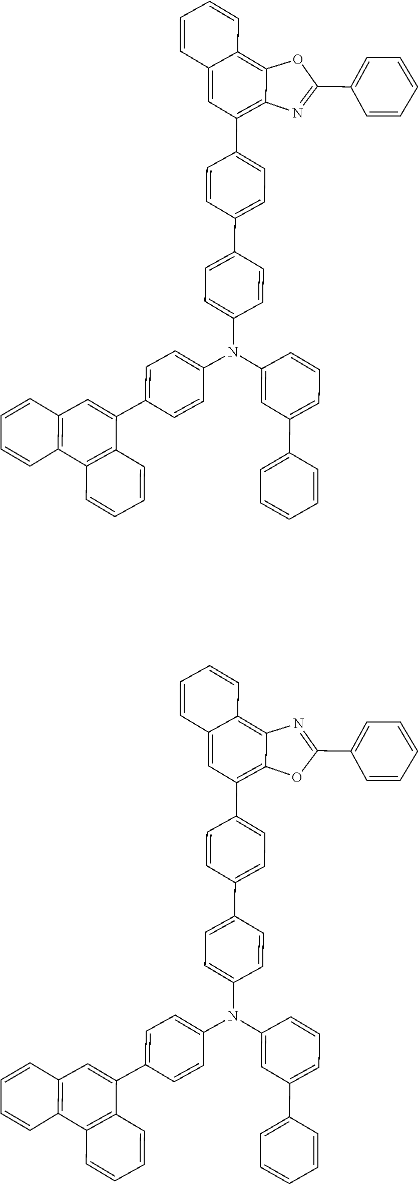 [SMILES: c1ccc(-c2cccc(N(c3ccc(-c4ccc(-c5cc6ccccc6c6oc(-c7ccccc7)nc56)cc4)cc3)c3ccc(-c4cc5ccccc5c5ccccc45)cc3)c2)cc1 ; c1ccc(-c2cccc(N(c3ccc(-c4ccc(-c5cc6ccccc6c6nc(-c7ccccc7)oc56)cc4)cc3)c3ccc(-c4cc5ccccc5c5ccccc45)cc3)c2)cc1]

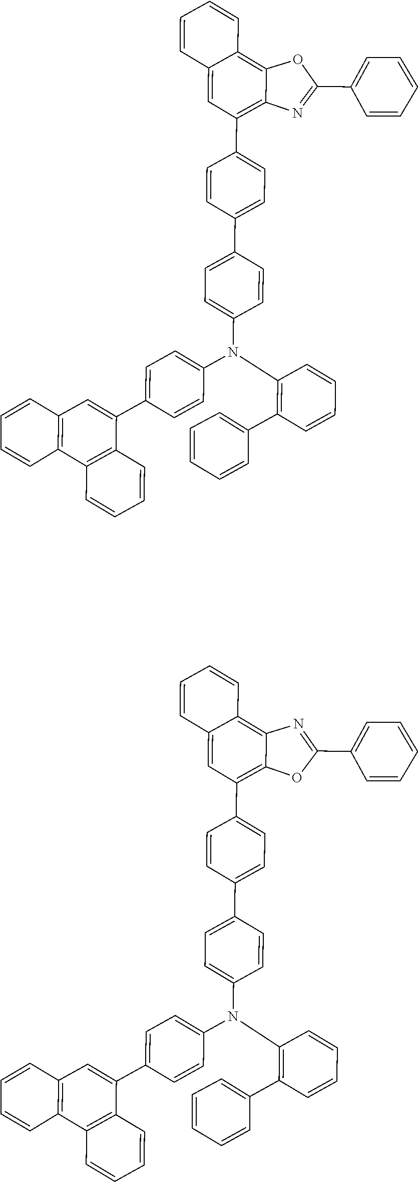 [SMILES: c1ccc(-c2nc3c(-c4ccc(-c5ccc(N(c6ccc(-c7cc8ccccc8c8ccccc78)cc6)c6ccccc6-c6ccccc6)cc5)cc4)cc4ccccc4c3o2)cc1 ; c1ccc(-c2nc3c(o2)c(-c2ccc(-c4ccc(N(c5ccc(-c6cc7ccccc7c7ccccc67)cc5)c5ccccc5-c5ccccc5)cc4)cc2)cc2ccccc23)cc1]

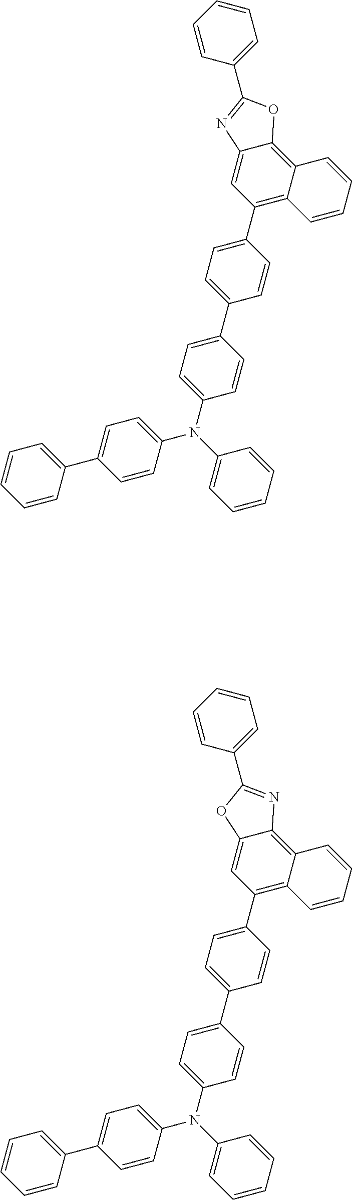 [SMILES: c1ccc(-c2ccc(N(c3ccccc3)c3ccc(-c4ccc(-c5cc6nc(-c7ccccc7)oc6c6ccccc56)cc4)cc3)cc2)cc1 ; c1ccc(-c2ccc(N(c3ccccc3)c3ccc(-c4ccc(-c5cc6oc(-c7ccccc7)nc6c6ccccc56)cc4)cc3)cc2)cc1]

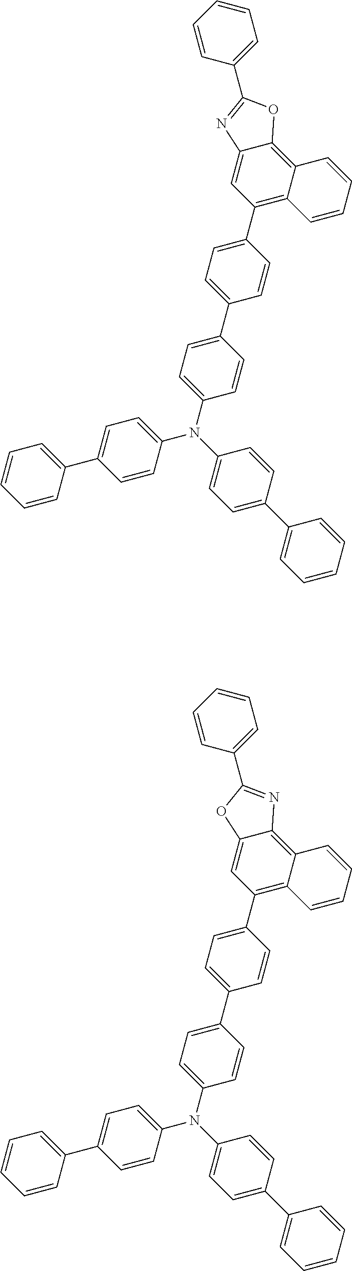 [SMILES: c1ccc(-c2ccc(N(c3ccc(-c4ccccc4)cc3)c3ccc(-c4ccc(-c5cc6nc(-c7ccccc7)oc6c6ccccc56)cc4)cc3)cc2)cc1 ; c1ccc(-c2ccc(N(c3ccc(-c4ccccc4)cc3)c3ccc(-c4ccc(-c5cc6oc(-c7ccccc7)nc6c6ccccc56)cc4)cc3)cc2)cc1]

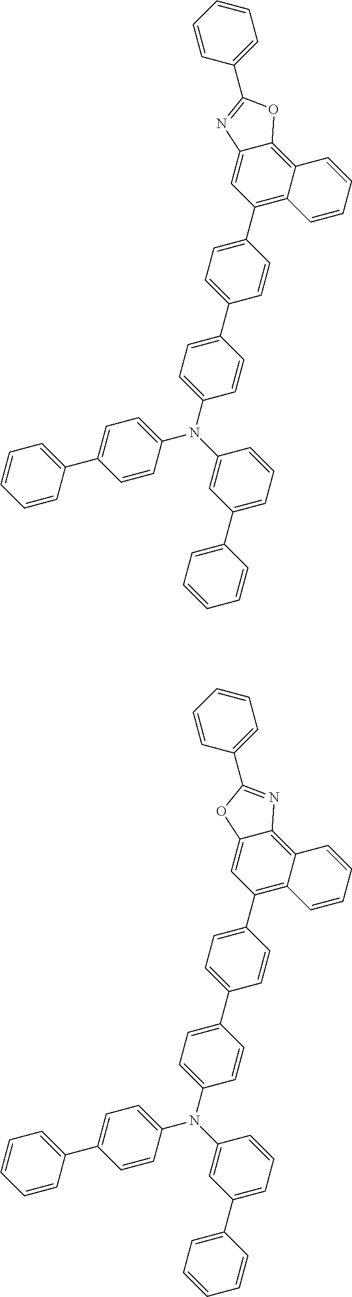 [SMILES: c1ccc(-c2ccc(N(c3ccc(-c4ccc(-c5cc6nc(-c7ccccc7)oc6c6ccccc56)cc4)cc3)c3cccc(-c4ccccc4)c3)cc2)cc1 ; c1ccc(-c2ccc(N(c3ccc(-c4ccc(-c5cc6oc(-c7ccccc7)nc6c6ccccc56)cc4)cc3)c3cccc(-c4ccccc4)c3)cc2)cc1]

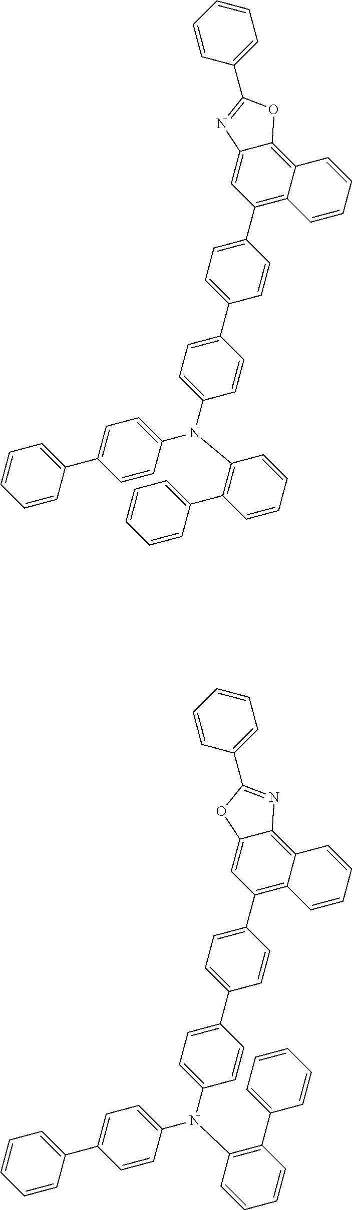 [SMILES: c1ccc(-c2ccc(N(c3ccc(-c4ccc(-c5cc6nc(-c7ccccc7)oc6c6ccccc56)cc4)cc3)c3ccccc3-c3ccccc3)cc2)cc1 ; c1ccc(-c2ccc(N(c3ccc(-c4ccc(-c5cc6oc(-c7ccccc7)nc6c6ccccc56)cc4)cc3)c3ccccc3-c3ccccc3)cc2)cc1]

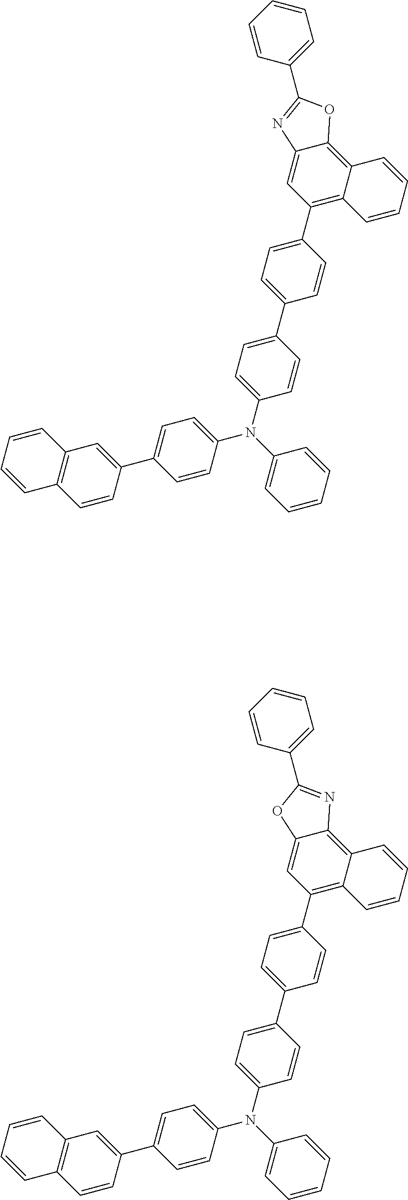 [SMILES: c1ccc(-c2nc3cc(-c4ccc(-c5ccc(N(c6ccccc6)c6ccc(-c7ccc8ccccc8c7)cc6)cc5)cc4)c4ccccc4c3o2)cc1 ; c1ccc(-c2nc3c(cc(-c4ccc(-c5ccc(N(c6ccccc6)c6ccc(-c7ccc8ccccc8c7)cc6)cc5)cc4)c4ccccc43)o2)cc1]

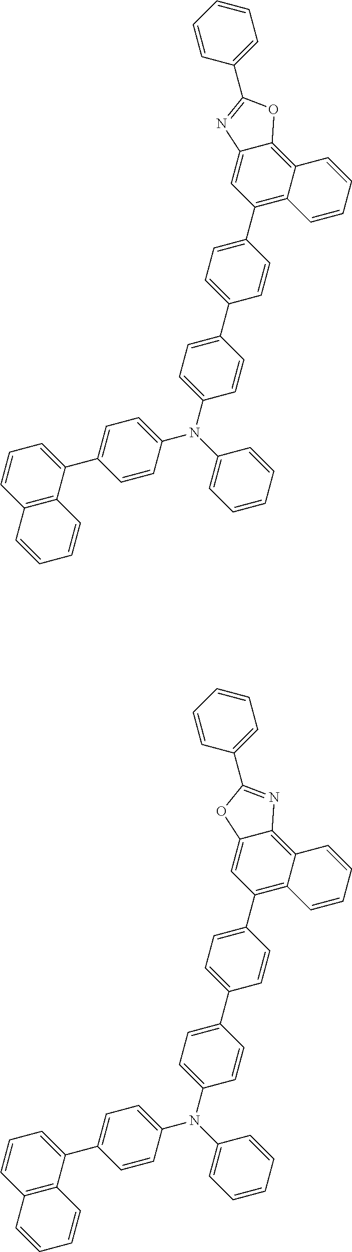 [SMILES: c1ccc(-c2nc3cc(-c4ccc(-c5ccc(N(c6ccccc6)c6ccc(-c7cccc8ccccc78)cc6)cc5)cc4)c4ccccc4c3o2)cc1 ; c1ccc(-c2nc3c(cc(-c4ccc(-c5ccc(N(c6ccccc6)c6ccc(-c7cccc8ccccc78)cc6)cc5)cc4)c4ccccc43)o2)cc1]

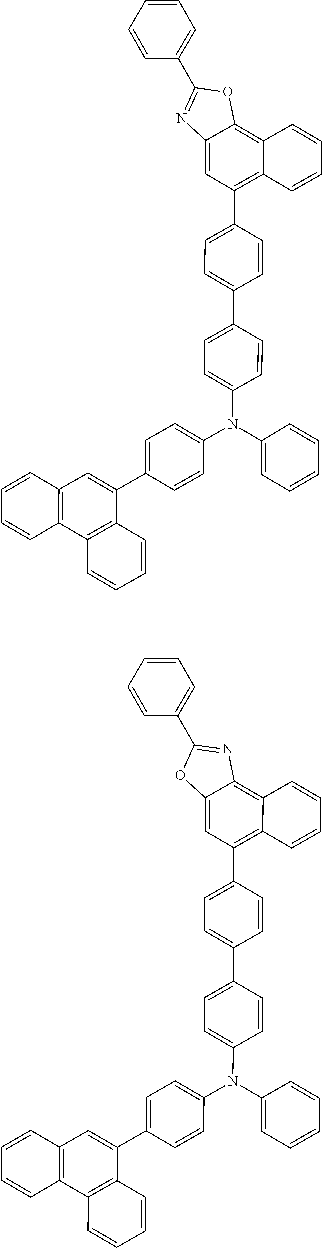 [SMILES: c1ccc(-c2nc3cc(-c4ccc(-c5ccc(N(c6ccccc6)c6ccc(-c7cc8ccccc8c8ccccc78)cc6)cc5)cc4)c4ccccc4c3o2)cc1 ; c1ccc(-c2nc3c(cc(-c4ccc(-c5ccc(N(c6ccccc6)c6ccc(-c7cc8ccccc8c8ccccc78)cc6)cc5)cc4)c4ccccc43)o2)cc1]

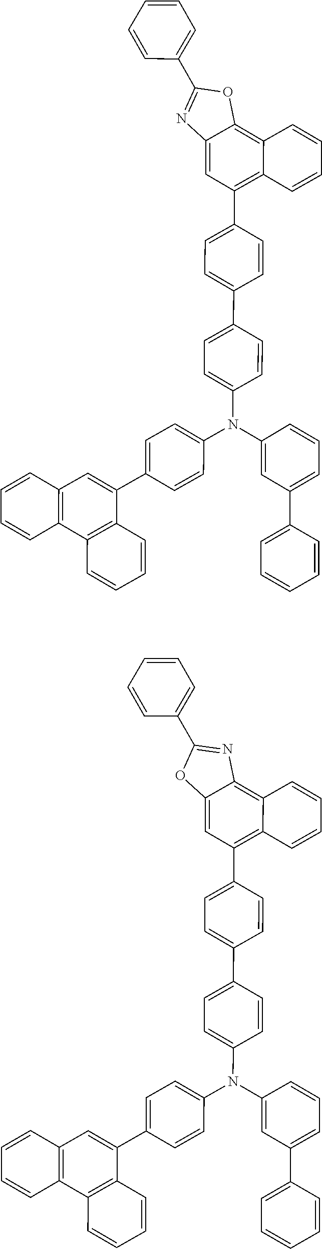 [SMILES: c1ccc(-c2cccc(N(c3ccc(-c4ccc(-c5cc6nc(-c7ccccc7)oc6c6ccccc56)cc4)cc3)c3ccc(-c4cc5ccccc5c5ccccc45)cc3)c2)cc1 ; c1ccc(-c2cccc(N(c3ccc(-c4ccc(-c5cc6oc(-c7ccccc7)nc6c6ccccc56)cc4)cc3)c3ccc(-c4cc5ccccc5c5ccccc45)cc3)c2)cc1]

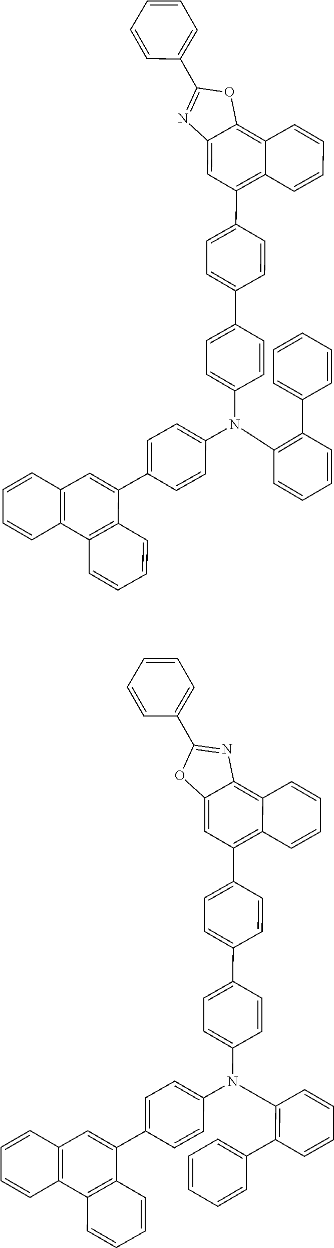 [SMILES: c1ccc(-c2nc3cc(-c4ccc(-c5ccc(N(c6ccc(-c7cc8ccccc8c8ccccc78)cc6)c6ccccc6-c6ccccc6)cc5)cc4)c4ccccc4c3o2)cc1 ; c1ccc(-c2nc3c(cc(-c4ccc(-c5ccc(N(c6ccc(-c7cc8ccccc8c8ccccc78)cc6)c6ccccc6-c6ccccc6)cc5)cc4)c4ccccc43)o2)cc1]

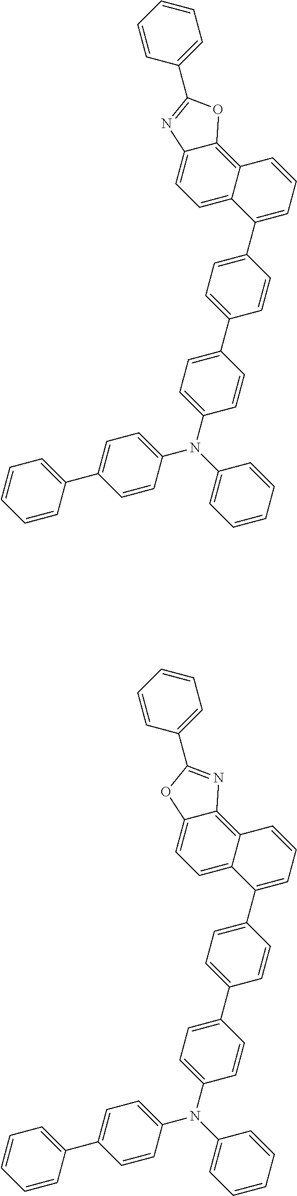 [SMILES: c1ccc(-c2ccc(N(c3ccccc3)c3ccc(-c4ccc(-c5cccc6c5ccc5nc(-c7ccccc7)oc56)cc4)cc3)cc2)cc1 ; c1ccc(-c2ccc(N(c3ccccc3)c3ccc(-c4ccc(-c5cccc6c5ccc5oc(-c7ccccc7)nc56)cc4)cc3)cc2)cc1]

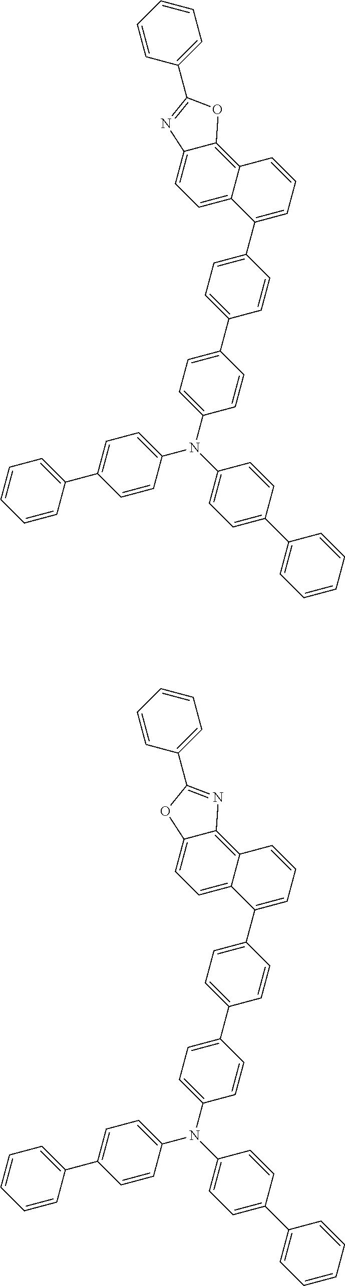 [SMILES: c1ccc(-c2ccc(N(c3ccc(-c4ccccc4)cc3)c3ccc(-c4ccc(-c5cccc6c5ccc5nc(-c7ccccc7)oc56)cc4)cc3)cc2)cc1 ; c1ccc(-c2ccc(N(c3ccc(-c4ccccc4)cc3)c3ccc(-c4ccc(-c5cccc6c5ccc5oc(-c7ccccc7)nc56)cc4)cc3)cc2)cc1]

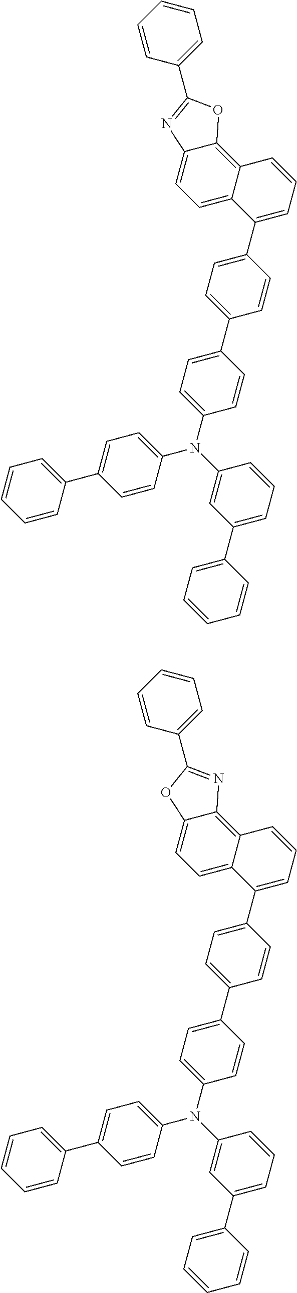 [SMILES: c1ccc(-c2ccc(N(c3ccc(-c4ccc(-c5cccc6c5ccc5nc(-c7ccccc7)oc56)cc4)cc3)c3cccc(-c4ccccc4)c3)cc2)cc1 ; c1ccc(-c2ccc(N(c3ccc(-c4ccc(-c5cccc6c5ccc5oc(-c7ccccc7)nc56)cc4)cc3)c3cccc(-c4ccccc4)c3)cc2)cc1]

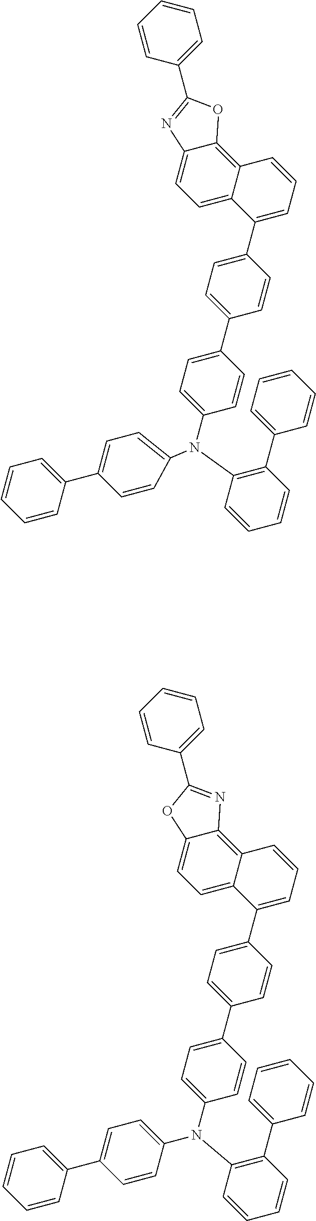 [SMILES: c1ccc(-c2ccc(N(c3ccc(-c4ccc(-c5cccc6c5ccc5nc(-c7ccccc7)oc56)cc4)cc3)c3ccccc3-c3ccccc3)cc2)cc1 ; c1ccc(-c2ccc(N(c3ccc(-c4ccc(-c5cccc6c5ccc5oc(-c7ccccc7)nc56)cc4)cc3)c3ccccc3-c3ccccc3)cc2)cc1]

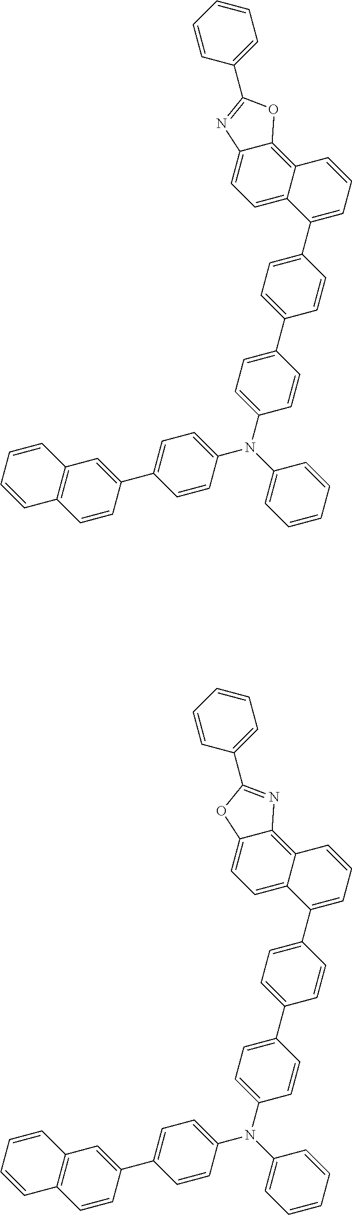 [SMILES: c1ccc(-c2nc3ccc4c(-c5ccc(-c6ccc(N(c7ccccc7)c7ccc(-c8ccc9ccccc9c8)cc7)cc6)cc5)cccc4c3o2)cc1 ; c1ccc(-c2nc3c(ccc4c(-c5ccc(-c6ccc(N(c7ccccc7)c7ccc(-c8ccc9ccccc9c8)cc7)cc6)cc5)cccc43)o2)cc1]

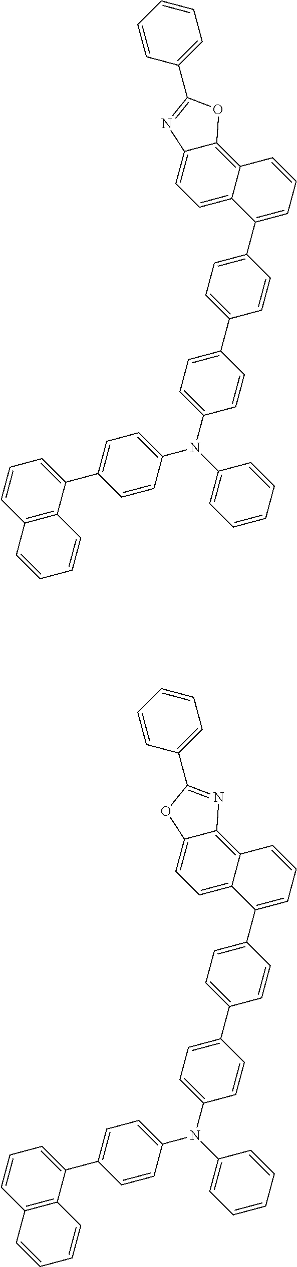 [SMILES: c1ccc(-c2nc3ccc4c(-c5ccc(-c6ccc(N(c7ccccc7)c7ccc(-c8cccc9ccccc89)cc7)cc6)cc5)cccc4c3o2)cc1 ; c1ccc(-c2nc3c(ccc4c(-c5ccc(-c6ccc(N(c7ccccc7)c7ccc(-c8cccc9ccccc89)cc7)cc6)cc5)cccc43)o2)cc1]

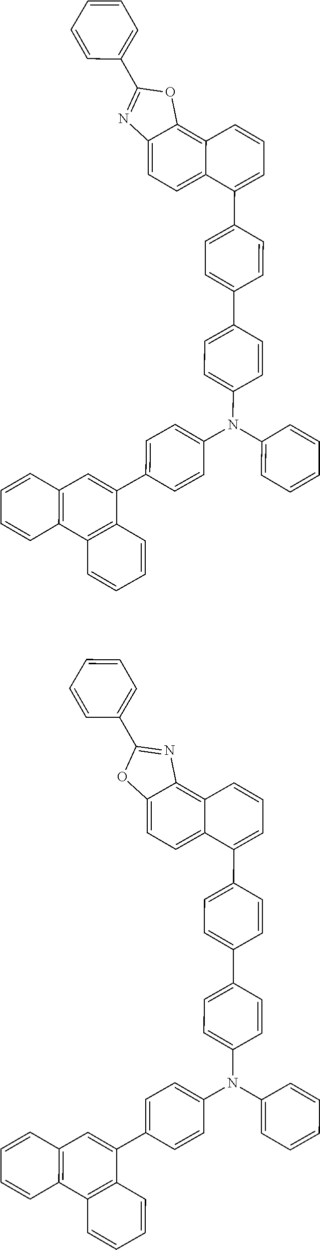 [SMILES: c1ccc(-c2nc3ccc4c(-c5ccc(-c6ccc(N(c7ccccc7)c7ccc(-c8cc9ccccc9c9ccccc89)cc7)cc6)cc5)cccc4c3o2)cc1 ; c1ccc(-c2nc3c(ccc4c(-c5ccc(-c6ccc(N(c7ccccc7)c7ccc(-c8cc9ccccc9c9ccccc89)cc7)cc6)cc5)cccc43)o2)cc1]

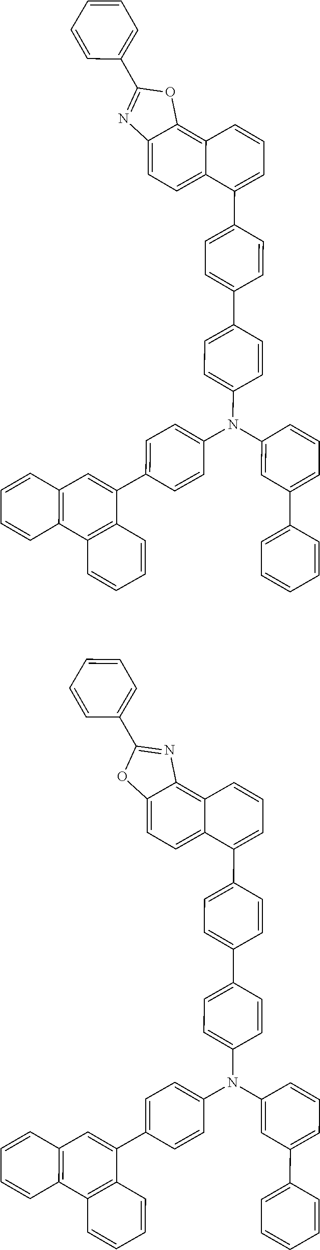 [SMILES: c1ccc(-c2cccc(N(c3ccc(-c4ccc(-c5cccc6c5ccc5nc(-c7ccccc7)oc56)cc4)cc3)c3ccc(-c4cc5ccccc5c5ccccc45)cc3)c2)cc1 ; c1ccc(-c2cccc(N(c3ccc(-c4ccc(-c5cccc6c5ccc5oc(-c7ccccc7)nc56)cc4)cc3)c3ccc(-c4cc5ccccc5c5ccccc45)cc3)c2)cc1]

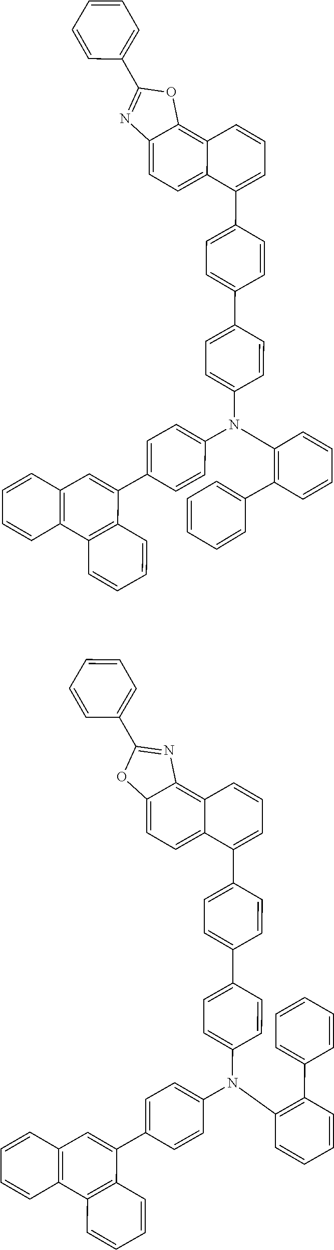 [SMILES: c1ccc(-c2nc3ccc4c(-c5ccc(-c6ccc(N(c7ccc(-c8cc9ccccc9c9ccccc89)cc7)c7ccccc7-c7ccccc7)cc6)cc5)cccc4c3o2)cc1 ; c1ccc(-c2nc3c(ccc4c(-c5ccc(-c6ccc(N(c7ccc(-c8cc9ccccc9c9ccccc89)cc7)c7ccccc7-c7ccccc7)cc6)cc5)cccc43)o2)cc1]

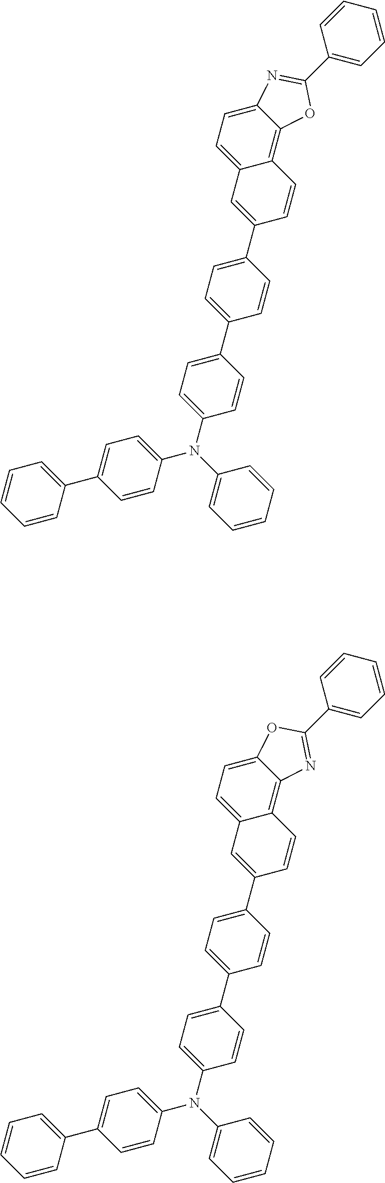 [SMILES: c1ccc(-c2ccc(N(c3ccccc3)c3ccc(-c4ccc(-c5ccc6c(ccc7nc(-c8ccccc8)oc76)c5)cc4)cc3)cc2)cc1 ; c1ccc(-c2ccc(N(c3ccccc3)c3ccc(-c4ccc(-c5ccc6c(ccc7oc(-c8ccccc8)nc76)c5)cc4)cc3)cc2)cc1]

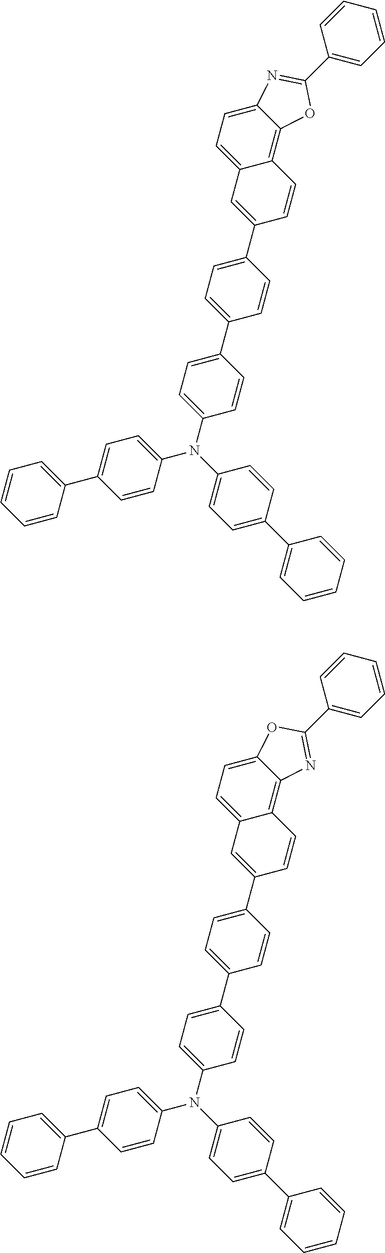 [SMILES: c1ccc(-c2ccc(N(c3ccc(-c4ccccc4)cc3)c3ccc(-c4ccc(-c5ccc6c(ccc7nc(-c8ccccc8)oc76)c5)cc4)cc3)cc2)cc1 ; c1ccc(-c2ccc(N(c3ccc(-c4ccccc4)cc3)c3ccc(-c4ccc(-c5ccc6c(ccc7oc(-c8ccccc8)nc76)c5)cc4)cc3)cc2)cc1]

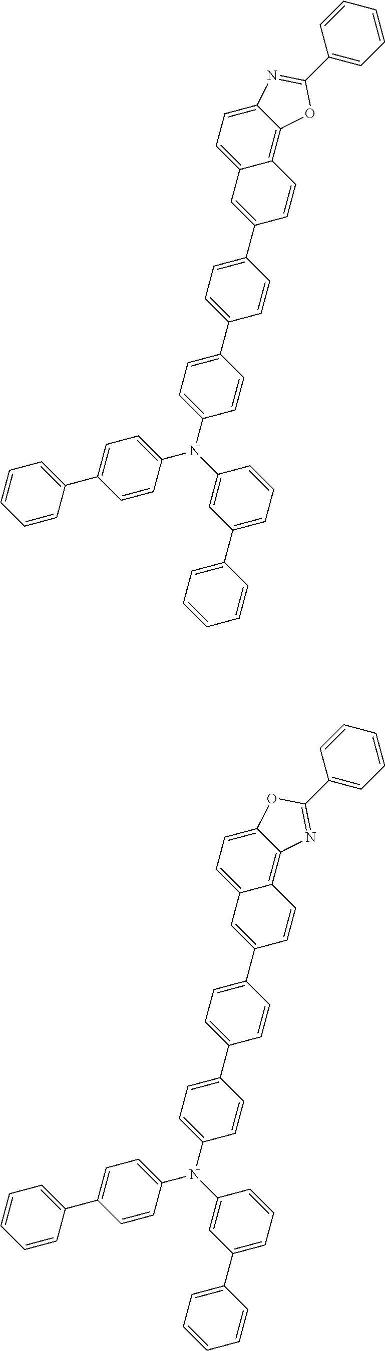 [SMILES: c1ccc(-c2ccc(N(c3ccc(-c4ccc(-c5ccc6c(ccc7nc(-c8ccccc8)oc76)c5)cc4)cc3)c3cccc(-c4ccccc4)c3)cc2)cc1 ; c1ccc(-c2ccc(N(c3ccc(-c4ccc(-c5ccc6c(ccc7oc(-c8ccccc8)nc76)c5)cc4)cc3)c3cccc(-c4ccccc4)c3)cc2)cc1]

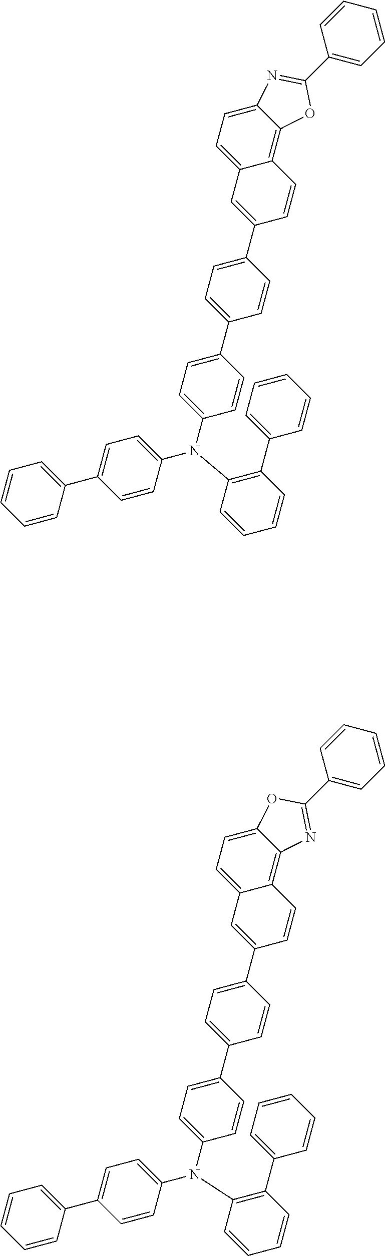 [SMILES: c1ccc(-c2ccc(N(c3ccc(-c4ccc(-c5ccc6c(ccc7nc(-c8ccccc8)oc76)c5)cc4)cc3)c3ccccc3-c3ccccc3)cc2)cc1 ; c1ccc(-c2ccc(N(c3ccc(-c4ccc(-c5ccc6c(ccc7oc(-c8ccccc8)nc76)c5)cc4)cc3)c3ccccc3-c3ccccc3)cc2)cc1]

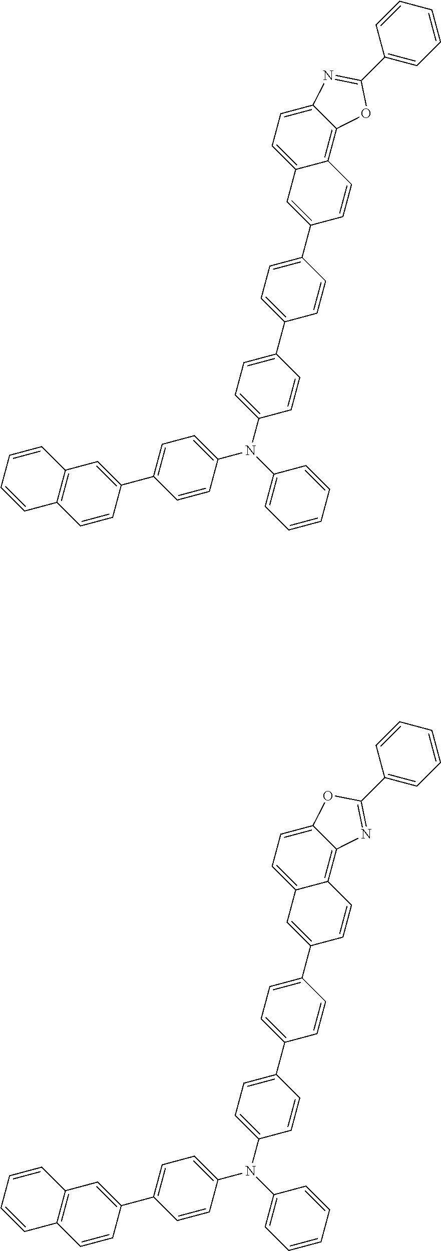 [SMILES: c1ccc(-c2nc3ccc4cc(-c5ccc(-c6ccc(N(c7ccccc7)c7ccc(-c8ccc9ccccc9c8)cc7)cc6)cc5)ccc4c3o2)cc1 ; c1ccc(-c2nc3c(ccc4cc(-c5ccc(-c6ccc(N(c7ccccc7)c7ccc(-c8ccc9ccccc9c8)cc7)cc6)cc5)ccc43)o2)cc1]

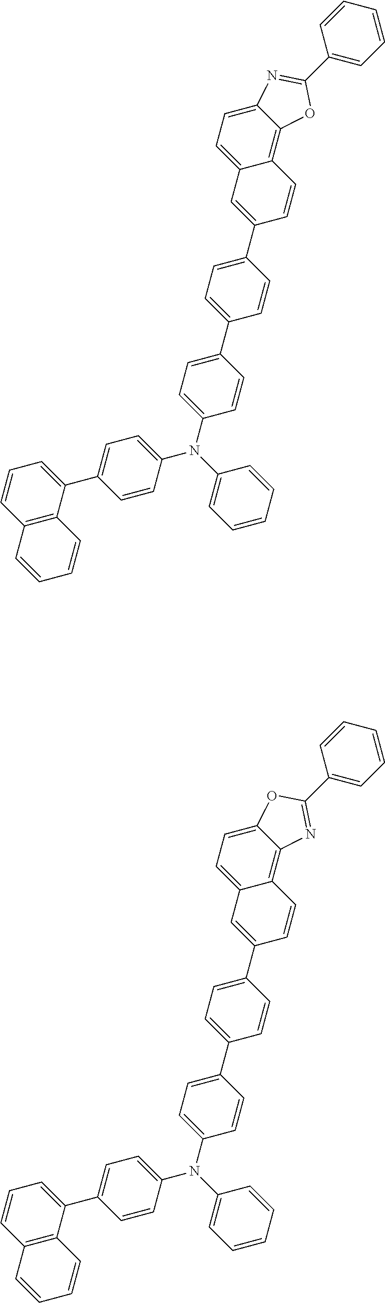 [SMILES: c1ccc(-c2nc3ccc4cc(-c5ccc(-c6ccc(N(c7ccccc7)c7ccc(-c8cccc9ccccc89)cc7)cc6)cc5)ccc4c3o2)cc1 ; c1ccc(-c2nc3c(ccc4cc(-c5ccc(-c6ccc(N(c7ccccc7)c7ccc(-c8cccc9ccccc89)cc7)cc6)cc5)ccc43)o2)cc1]

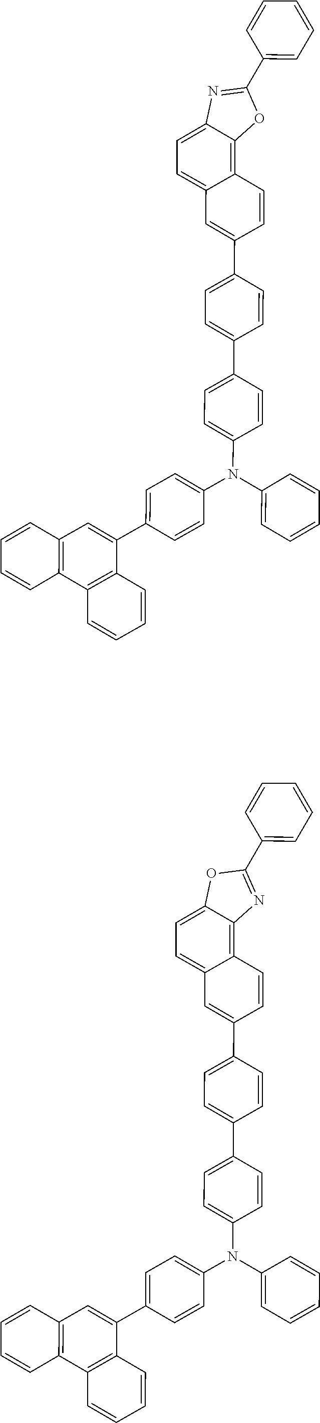 [SMILES: c1ccc(-c2nc3ccc4cc(-c5ccc(-c6ccc(N(c7ccccc7)c7ccc(-c8cc9ccccc9c9ccccc89)cc7)cc6)cc5)ccc4c3o2)cc1 ; c1ccc(-c2nc3c(ccc4cc(-c5ccc(-c6ccc(N(c7ccccc7)c7ccc(-c8cc9ccccc9c9ccccc89)cc7)cc6)cc5)ccc43)o2)cc1]

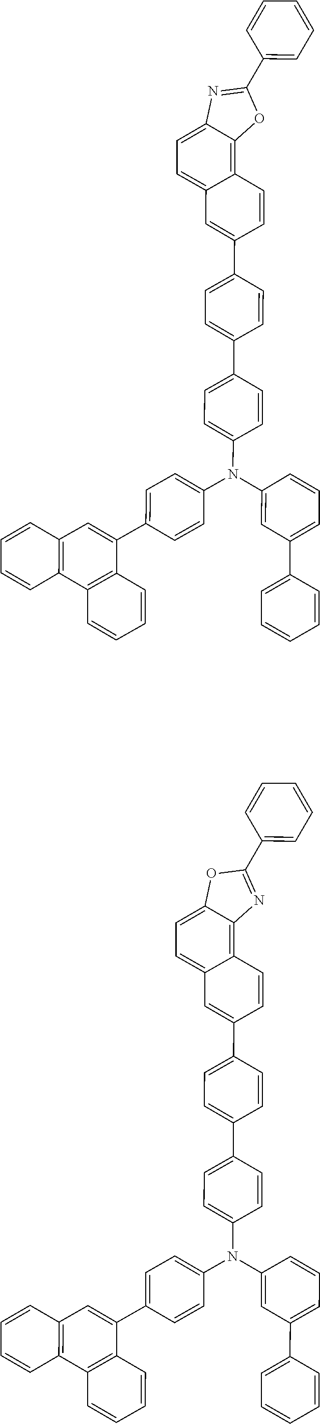 [SMILES: c1ccc(-c2cccc(N(c3ccc(-c4ccc(-c5ccc6c(ccc7nc(-c8ccccc8)oc76)c5)cc4)cc3)c3ccc(-c4cc5ccccc5c5ccccc45)cc3)c2)cc1 ; c1ccc(-c2cccc(N(c3ccc(-c4ccc(-c5ccc6c(ccc7oc(-c8ccccc8)nc76)c5)cc4)cc3)c3ccc(-c4cc5ccccc5c5ccccc45)cc3)c2)cc1]

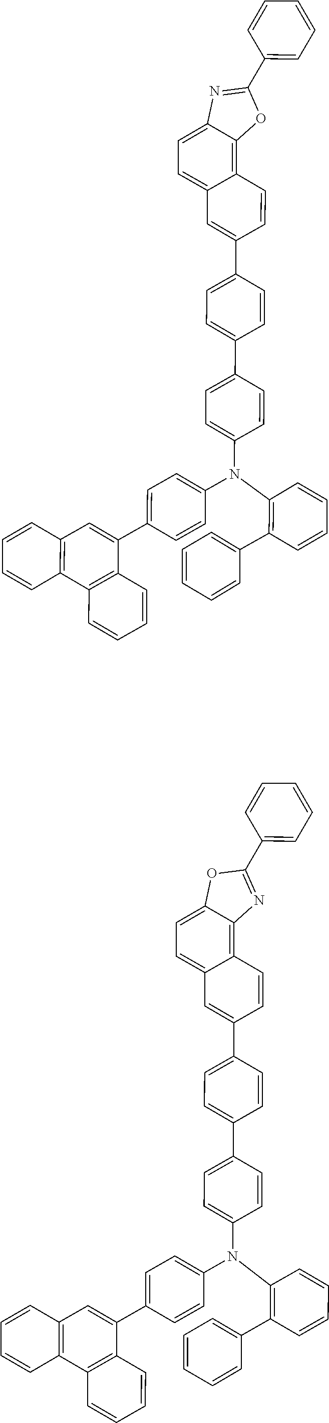 [SMILES: c1ccc(-c2nc3ccc4cc(-c5ccc(-c6ccc(N(c7ccc(-c8cc9ccccc9c9ccccc89)cc7)c7ccccc7-c7ccccc7)cc6)cc5)ccc4c3o2)cc1 ; c1ccc(-c2nc3c(ccc4cc(-c5ccc(-c6ccc(N(c7ccc(-c8cc9ccccc9c9ccccc89)cc7)c7ccccc7-c7ccccc7)cc6)cc5)ccc43)o2)cc1]

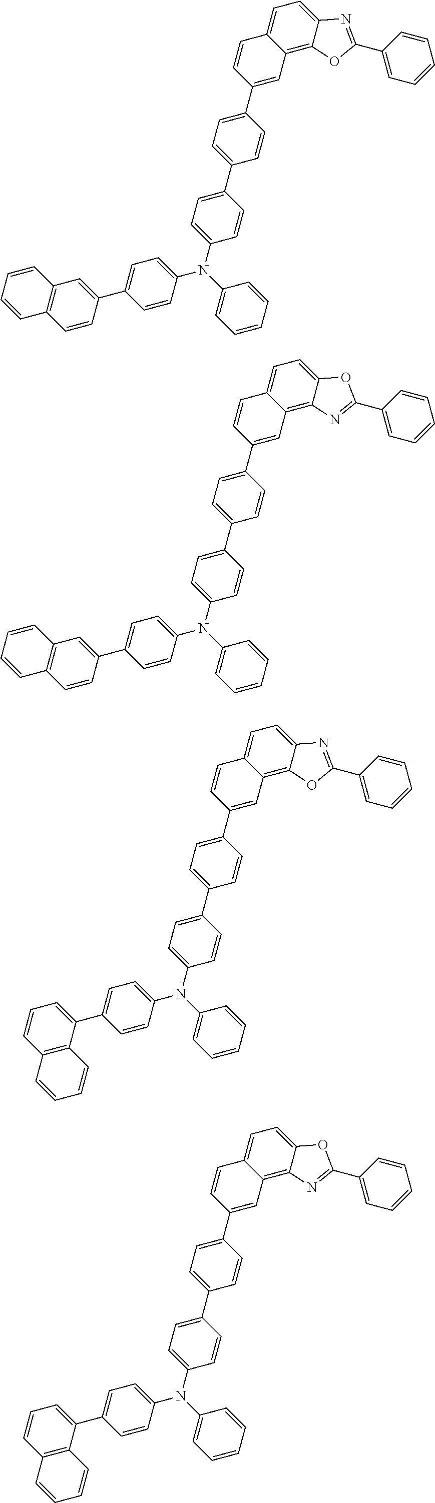 [SMILES: c1ccc(-c2nc3ccc4ccc(-c5ccc(-c6ccc(N(c7ccccc7)c7ccc(-c8ccc9ccccc9c8)cc7)cc6)cc5)cc4c3o2)cc1 ; c1ccc(-c2nc3c(ccc4ccc(-c5ccc(-c6ccc(N(c7ccccc7)c7ccc(-c8ccc9ccccc9c8)cc7)cc6)cc5)cc43)o2)cc1 ; c1ccc(-c2nc3ccc4ccc(-c5ccc(-c6ccc(N(c7ccccc7)c7ccc(-c8cccc9ccccc89)cc7)cc6)cc5)cc4c3o2)cc1 ; c1ccc(-c2nc3c(ccc4ccc(-c5ccc(-c6ccc(N(c7ccccc7)c7ccc(-c8cccc9ccccc89)cc7)cc6)cc5)cc43)o2)cc1]

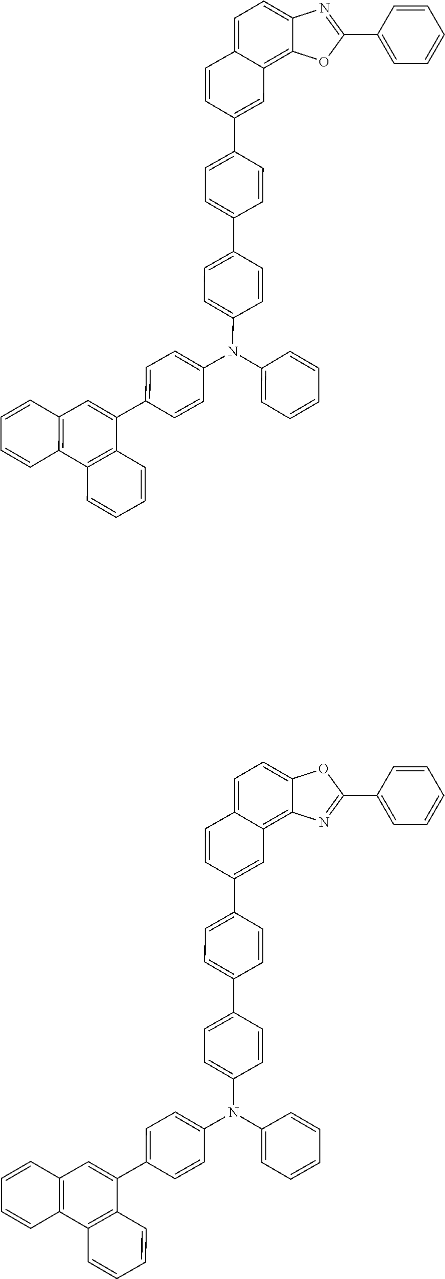 [SMILES: c1ccc(-c2nc3ccc4ccc(-c5ccc(-c6ccc(N(c7ccccc7)c7ccc(-c8cc9ccccc9c9ccccc89)cc7)cc6)cc5)cc4c3o2)cc1 ; c1ccc(-c2nc3c(ccc4ccc(-c5ccc(-c6ccc(N(c7ccccc7)c7ccc(-c8cc9ccccc9c9ccccc89)cc7)cc6)cc5)cc43)o2)cc1]

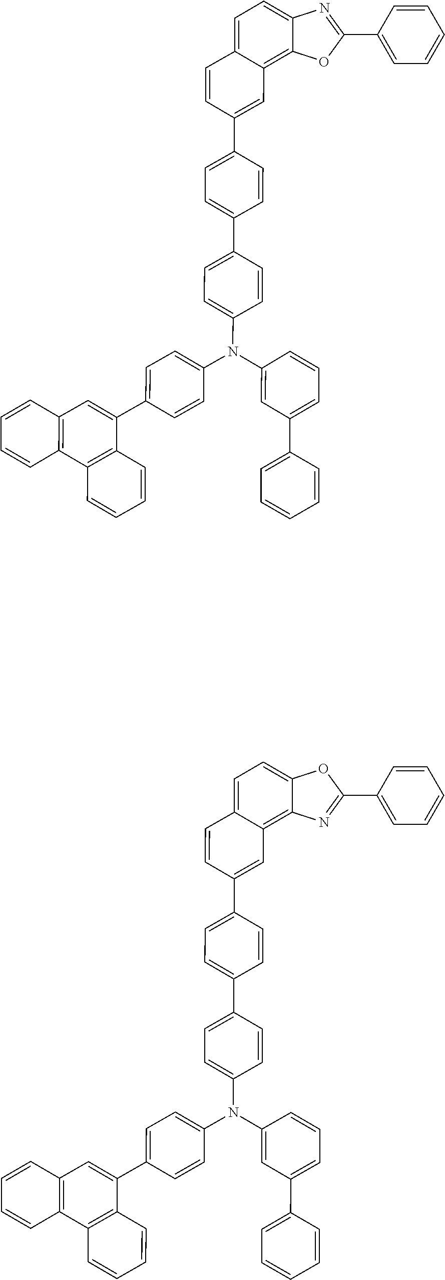 [SMILES: c1ccc(-c2cccc(N(c3ccc(-c4ccc(-c5ccc6ccc7nc(-c8ccccc8)oc7c6c5)cc4)cc3)c3ccc(-c4cc5ccccc5c5ccccc45)cc3)c2)cc1 ; c1ccc(-c2cccc(N(c3ccc(-c4ccc(-c5ccc6ccc7oc(-c8ccccc8)nc7c6c5)cc4)cc3)c3ccc(-c4cc5ccccc5c5ccccc45)cc3)c2)cc1]

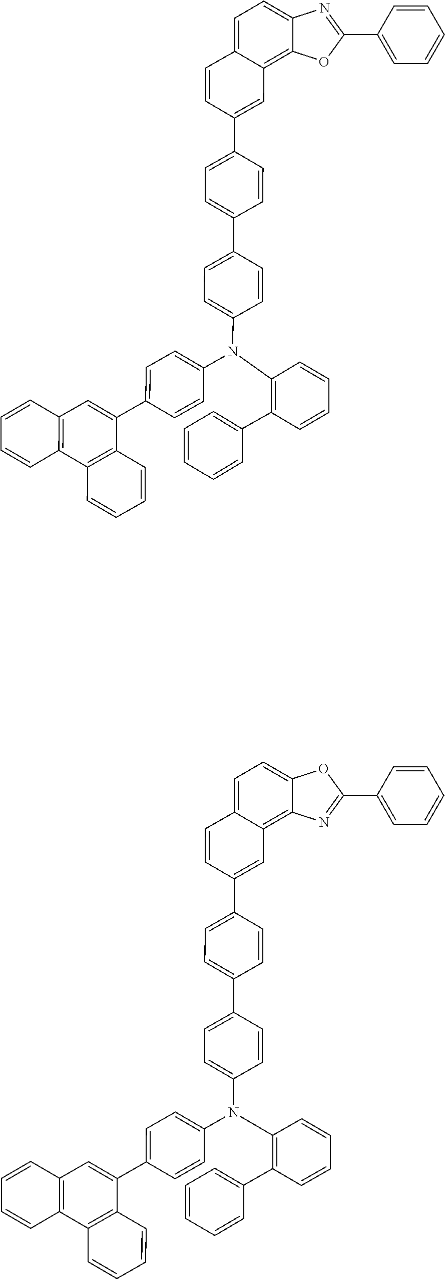 [SMILES: c1ccc(-c2nc3ccc4ccc(-c5ccc(-c6ccc(N(c7ccc(-c8cc9ccccc9c9ccccc89)cc7)c7ccccc7-c7ccccc7)cc6)cc5)cc4c3o2)cc1 ; c1ccc(-c2nc3c(ccc4ccc(-c5ccc(-c6ccc(N(c7ccc(-c8cc9ccccc9c9ccccc89)cc7)c7ccccc7-c7ccccc7)cc6)cc5)cc43)o2)cc1]

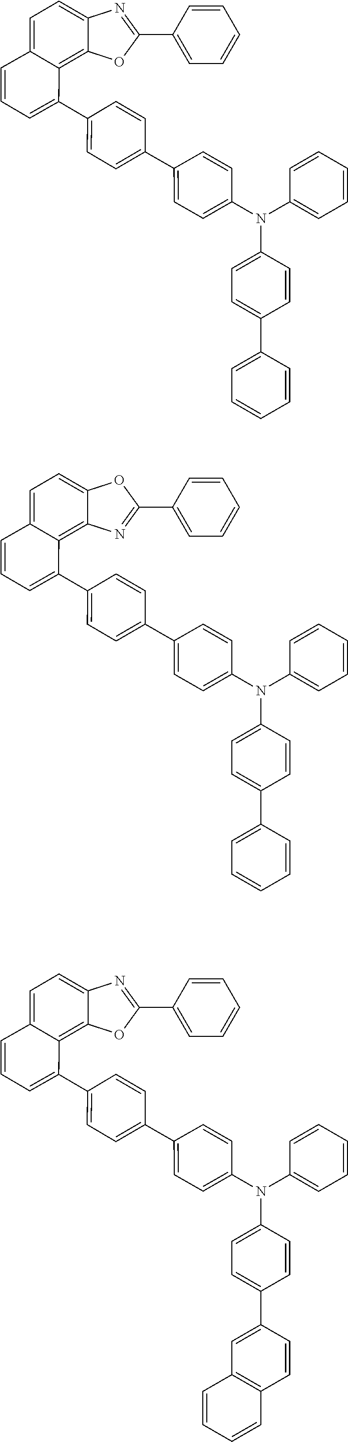 [SMILES: c1ccc(-c2ccc(N(c3ccccc3)c3ccc(-c4ccc(-c5cccc6ccc7nc(-c8ccccc8)oc7c56)cc4)cc3)cc2)cc1 ; c1ccc(-c2ccc(N(c3ccccc3)c3ccc(-c4ccc(-c5cccc6ccc7oc(-c8ccccc8)nc7c56)cc4)cc3)cc2)cc1 ; c1ccc(-c2nc3ccc4cccc(-c5ccc(-c6ccc(N(c7ccccc7)c7ccc(-c8ccc9ccccc9c8)cc7)cc6)cc5)c4c3o2)cc1]

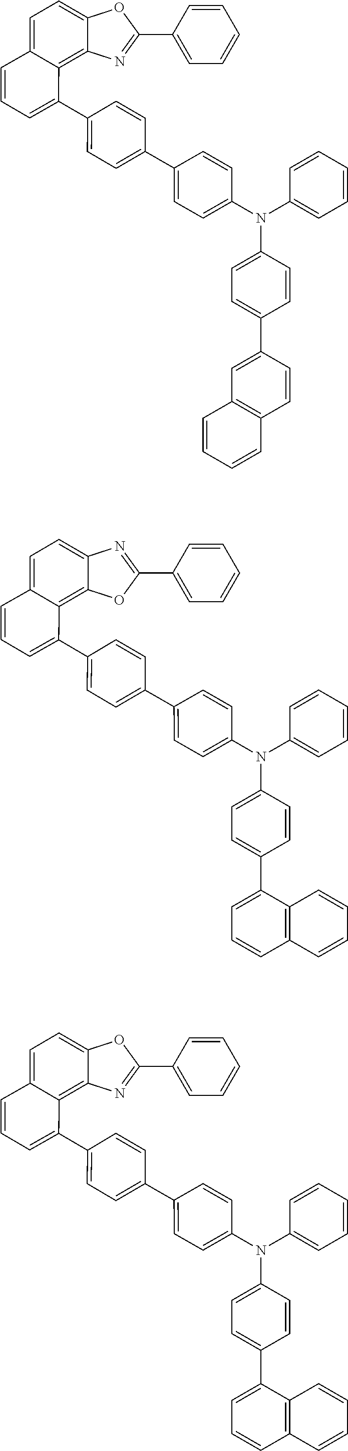 [SMILES: c1ccc(-c2nc3c(ccc4cccc(-c5ccc(-c6ccc(N(c7ccccc7)c7ccc(-c8ccc9ccccc9c8)cc7)cc6)cc5)c43)o2)cc1 ; c1ccc(-c2nc3ccc4cccc(-c5ccc(-c6ccc(N(c7ccccc7)c7ccc(-c8cccc9ccccc89)cc7)cc6)cc5)c4c3o2)cc1 ; c1ccc(-c2nc3c(ccc4cccc(-c5ccc(-c6ccc(N(c7ccccc7)c7ccc(-c8cccc9ccccc89)cc7)cc6)cc5)c43)o2)cc1]

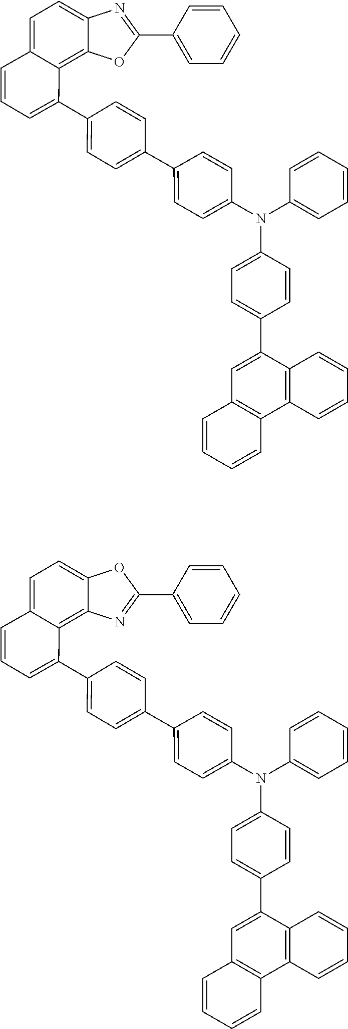 [SMILES: c1ccc(-c2nc3ccc4cccc(-c5ccc(-c6ccc(N(c7ccccc7)c7ccc(-c8cc9ccccc9c9ccccc89)cc7)cc6)cc5)c4c3o2)cc1 ; c1ccc(-c2nc3c(ccc4cccc(-c5ccc(-c6ccc(N(c7ccccc7)c7ccc(-c8cc9ccccc9c9ccccc89)cc7)cc6)cc5)c43)o2)cc1]

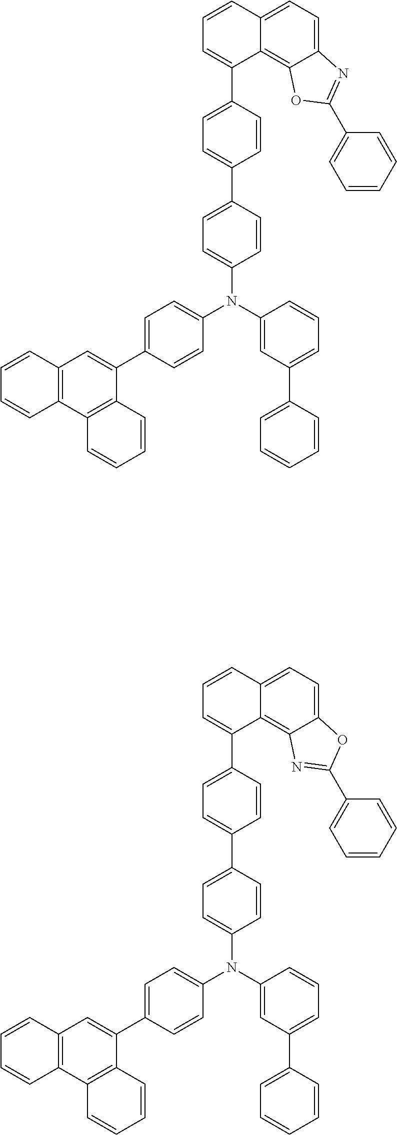 [SMILES: c1ccc(-c2cccc(N(c3ccc(-c4ccc(-c5cccc6ccc7nc(-c8ccccc8)oc7c56)cc4)cc3)c3ccc(-c4cc5ccccc5c5ccccc45)cc3)c2)cc1 ; c1ccc(-c2cccc(N(c3ccc(-c4ccc(-c5cccc6ccc7oc(-c8ccccc8)nc7c56)cc4)cc3)c3ccc(-c4cc5ccccc5c5ccccc45)cc3)c2)cc1]

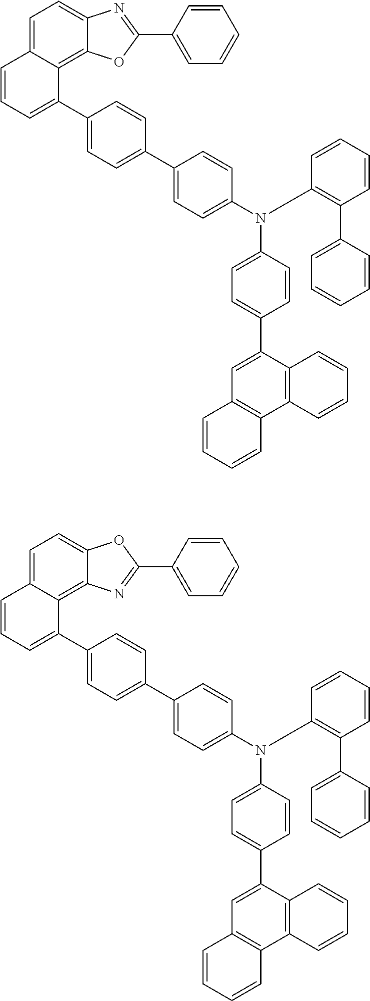 [SMILES: c1ccc(-c2nc3ccc4cccc(-c5ccc(-c6ccc(N(c7ccc(-c8cc9ccccc9c9ccccc89)cc7)c7ccccc7-c7ccccc7)cc6)cc5)c4c3o2)cc1 ; c1ccc(-c2nc3c(ccc4cccc(-c5ccc(-c6ccc(N(c7ccc(-c8cc9ccccc9c9ccccc89)cc7)c7ccccc7-c7ccccc7)cc6)cc5)c43)o2)cc1]

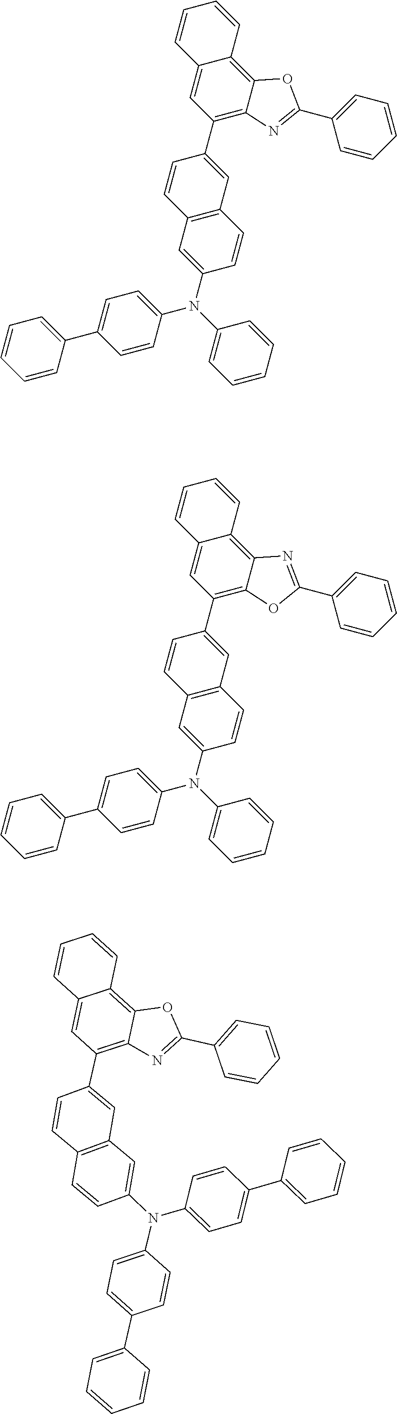 [SMILES: c1ccc(-c2ccc(N(c3ccccc3)c3ccc4cc(-c5cc6ccccc6c6oc(-c7ccccc7)nc56)ccc4c3)cc2)cc1 ; c1ccc(-c2ccc(N(c3ccccc3)c3ccc4cc(-c5cc6ccccc6c6nc(-c7ccccc7)oc56)ccc4c3)cc2)cc1 ; c1ccc(-c2ccc(N(c3ccc(-c4ccccc4)cc3)c3ccc4ccc(-c5cc6ccccc6c6oc(-c7ccccc7)nc56)cc4c3)cc2)cc1]

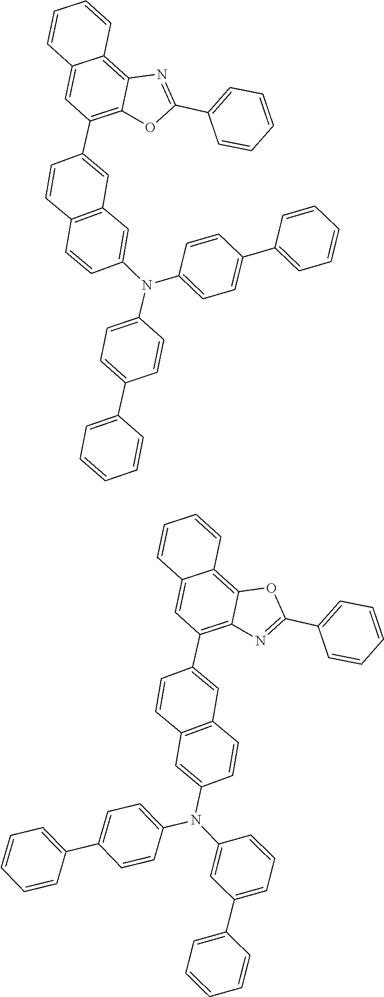 [SMILES: c1ccc(-c2ccc(N(c3ccc(-c4ccccc4)cc3)c3ccc4ccc(-c5cc6ccccc6c6nc(-c7ccccc7)oc56)cc4c3)cc2)cc1 ; c1ccc(-c2ccc(N(c3cccc(-c4ccccc4)c3)c3ccc4cc(-c5cc6ccccc6c6oc(-c7ccccc7)nc56)ccc4c3)cc2)cc1]

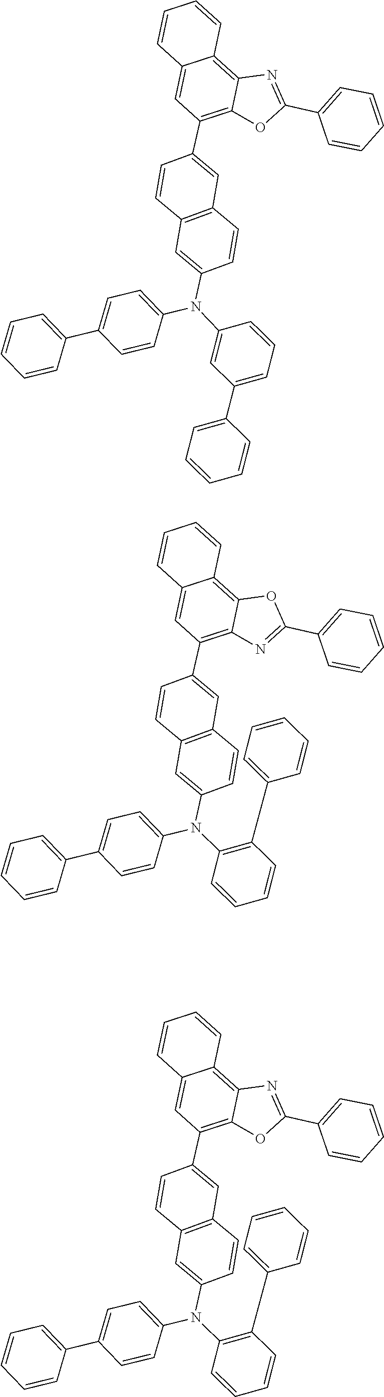

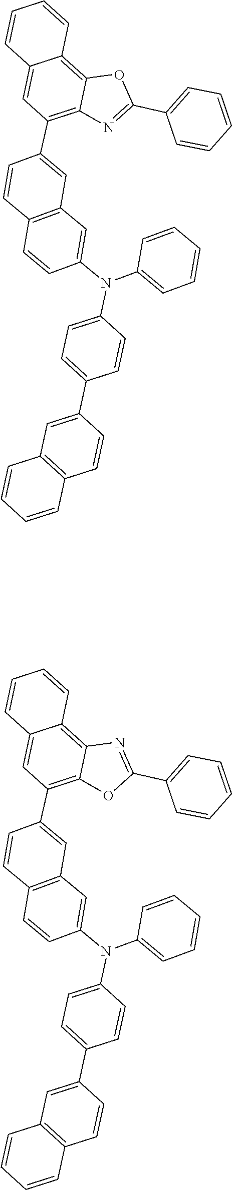 [SMILES: c1ccc(-c2nc3c(-c4ccc5ccc(N(c6ccccc6)c6ccc(-c7ccc8ccccc8c7)cc6)cc5c4)cc4ccccc4c3o2)cc1 ; c1ccc(-c2nc3c(o2)c(-c2ccc4ccc(N(c5ccccc5)c5ccc(-c6ccc7ccccc7c6)cc5)cc4c2)cc2ccccc23)cc1]

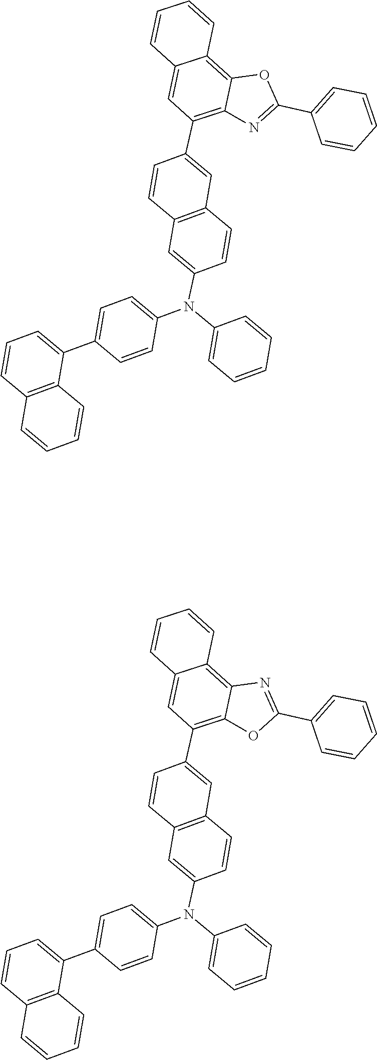 [SMILES: c1ccc(-c2nc3c(-c4ccc5cc(N(c6ccccc6)c6ccc(-c7cccc8ccccc78)cc6)ccc5c4)cc4ccccc4c3o2)cc1 ; c1ccc(-c2nc3c(o2)c(-c2ccc4cc(N(c5ccccc5)c5ccc(-c6cccc7ccccc67)cc5)ccc4c2)cc2ccccc23)cc1]

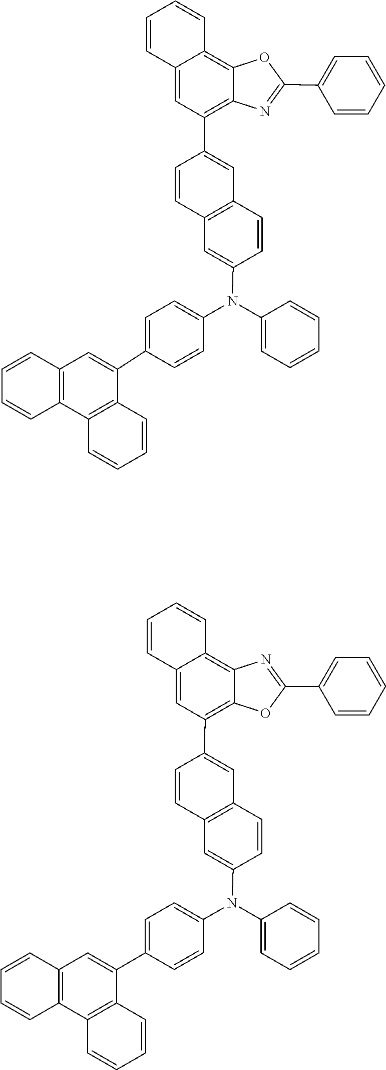 [SMILES: c1ccc(-c2nc3c(-c4ccc5cc(N(c6ccccc6)c6ccc(-c7cc8ccccc8c8ccccc78)cc6)ccc5c4)cc4ccccc4c3o2)cc1 ; c1ccc(-c2nc3c(o2)c(-c2ccc4cc(N(c5ccccc5)c5ccc(-c6cc7ccccc7c7ccccc67)cc5)ccc4c2)cc2ccccc23)cc1]

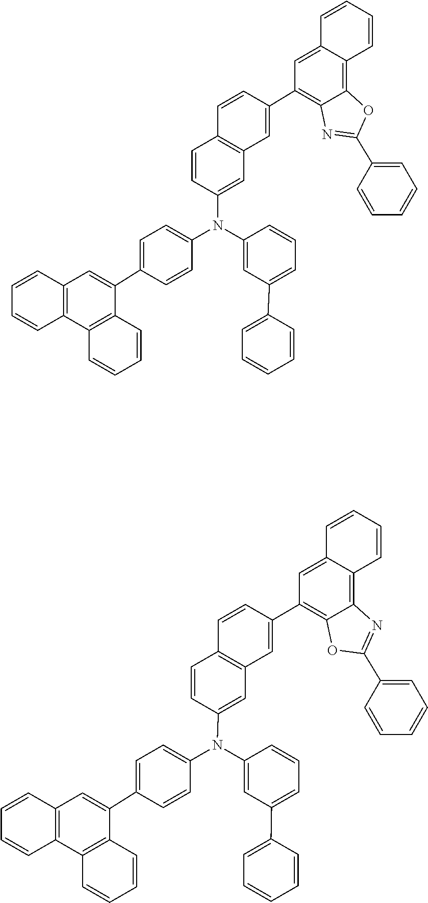 [SMILES: c1ccc(-c2cccc(N(c3ccc(-c4cc5ccccc5c5ccccc45)cc3)c3ccc4ccc(-c5cc6ccccc6c6oc(-c7ccccc7)nc56)cc4c3)c2)cc1 ; c1ccc(-c2cccc(N(c3ccc(-c4cc5ccccc5c5ccccc45)cc3)c3ccc4ccc(-c5cc6ccccc6c6nc(-c7ccccc7)oc56)cc4c3)c2)cc1]

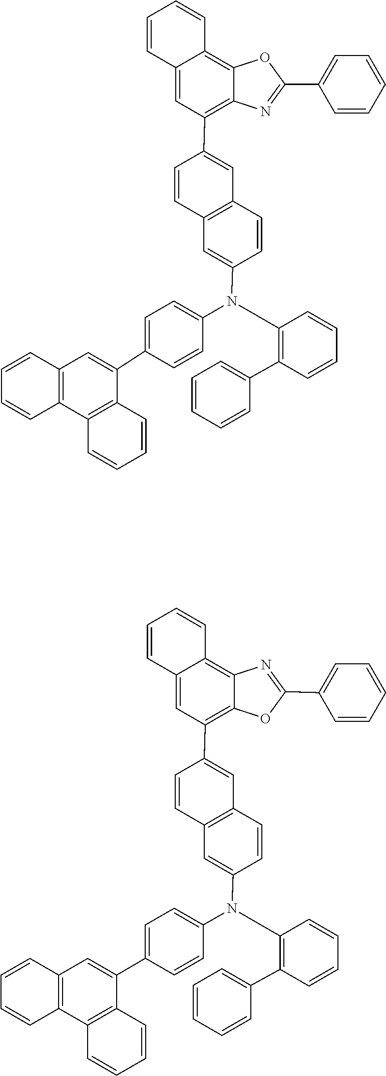 [SMILES: c1ccc(-c2nc3c(-c4ccc5cc(N(c6ccc(-c7cc8ccccc8c8ccccc78)cc6)c6ccccc6-c6ccccc6)ccc5c4)cc4ccccc4c3o2)cc1 ; c1ccc(-c2nc3c(o2)c(-c2ccc4cc(N(c5ccc(-c6cc7ccccc7c7ccccc67)cc5)c5ccccc5-c5ccccc5)ccc4c2)cc2ccccc23)cc1]

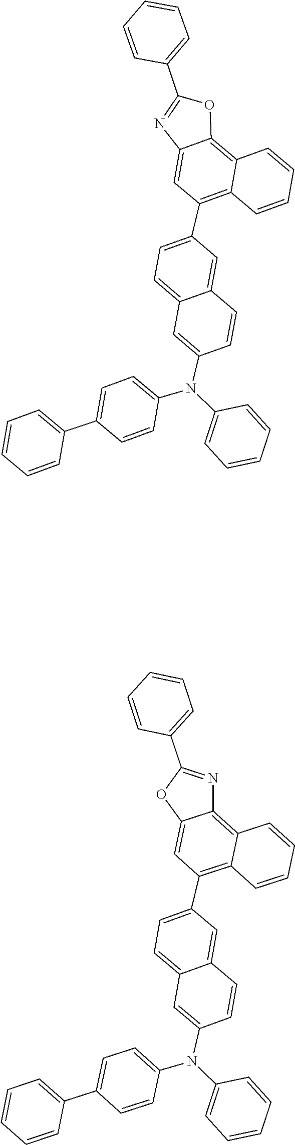 [SMILES: c1ccc(-c2ccc(N(c3ccccc3)c3ccc4cc(-c5cc6nc(-c7ccccc7)oc6c6ccccc56)ccc4c3)cc2)cc1 ; c1ccc(-c2ccc(N(c3ccccc3)c3ccc4cc(-c5cc6oc(-c7ccccc7)nc6c6ccccc56)ccc4c3)cc2)cc1]

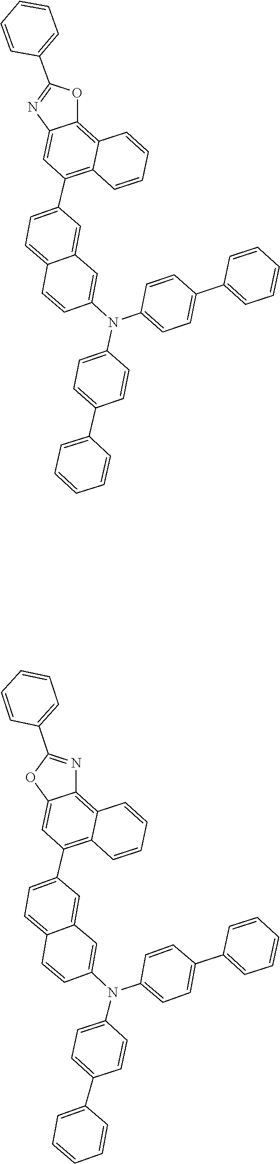 [SMILES: c1ccc(-c2ccc(N(c3ccc(-c4ccccc4)cc3)c3ccc4ccc(-c5cc6nc(-c7ccccc7)oc6c6ccccc56)cc4c3)cc2)cc1 ; c1ccc(-c2ccc(N(c3ccc(-c4ccccc4)cc3)c3ccc4ccc(-c5cc6oc(-c7ccccc7)nc6c6ccccc56)cc4c3)cc2)cc1]

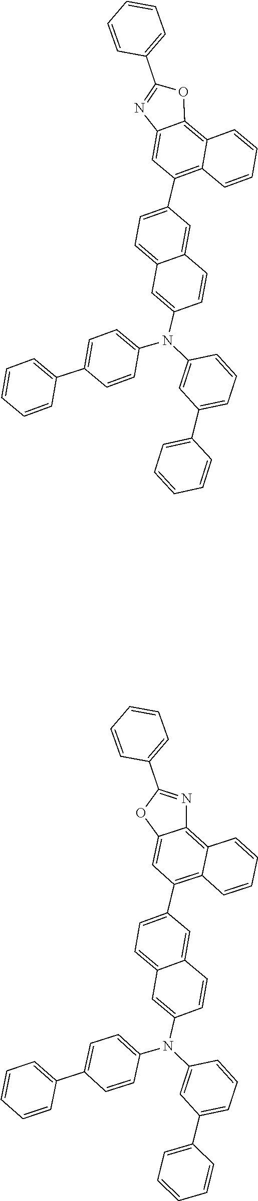 [SMILES: c1ccc(-c2ccc(N(c3cccc(-c4ccccc4)c3)c3ccc4cc(-c5cc6nc(-c7ccccc7)oc6c6ccccc56)ccc4c3)cc2)cc1 ; c1ccc(-c2ccc(N(c3cccc(-c4ccccc4)c3)c3ccc4cc(-c5cc6oc(-c7ccccc7)nc6c6ccccc56)ccc4c3)cc2)cc1]

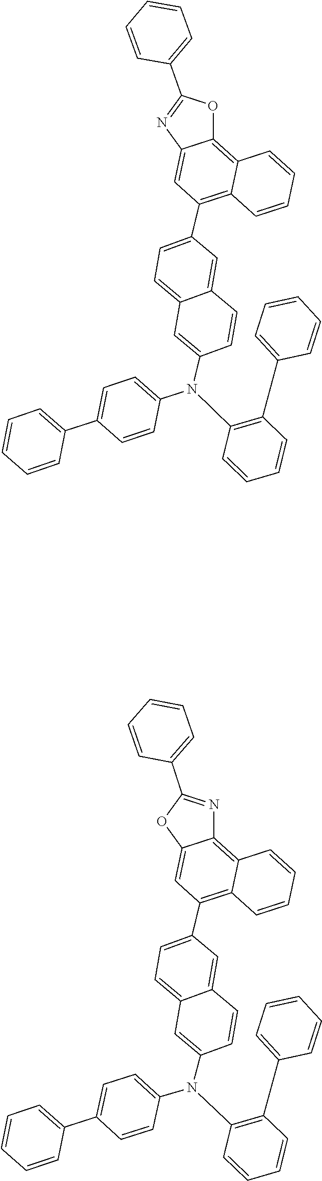 [SMILES: c1ccc(-c2ccc(N(c3ccc4cc(-c5cc6nc(-c7ccccc7)oc6c6ccccc56)ccc4c3)c3ccccc3-c3ccccc3)cc2)cc1 ; c1ccc(-c2ccc(N(c3ccc4cc(-c5cc6oc(-c7ccccc7)nc6c6ccccc56)ccc4c3)c3ccccc3-c3ccccc3)cc2)cc1]

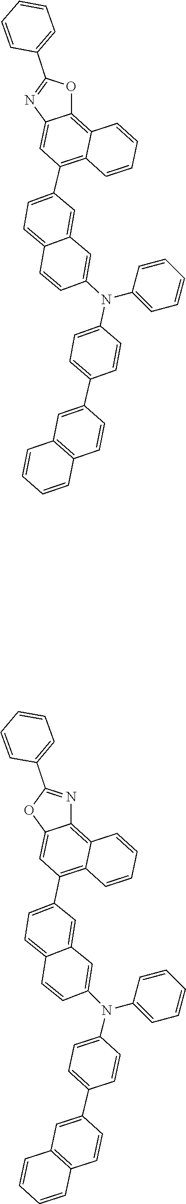 [SMILES: c1ccc(-c2nc3cc(-c4ccc5ccc(N(c6ccccc6)c6ccc(-c7ccc8ccccc8c7)cc6)cc5c4)c4ccccc4c3o2)cc1 ; c1ccc(-c2nc3c(cc(-c4ccc5ccc(N(c6ccccc6)c6ccc(-c7ccc8ccccc8c7)cc6)cc5c4)c4ccccc43)o2)cc1]

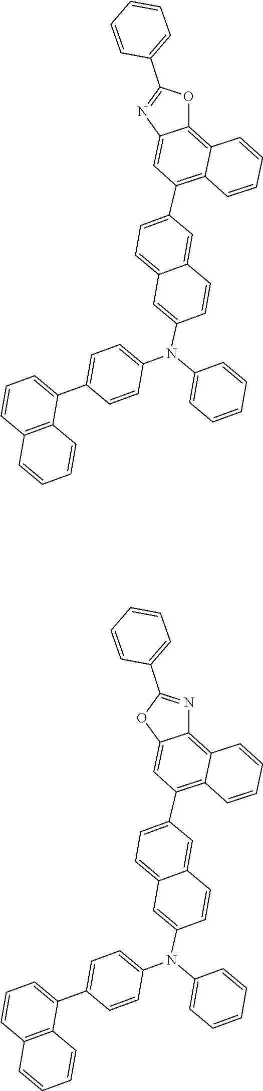 [SMILES: c1ccc(-c2nc3cc(-c4ccc5cc(N(c6ccccc6)c6ccc(-c7cccc8ccccc78)cc6)ccc5c4)c4ccccc4c3o2)cc1 ; c1ccc(-c2nc3c(cc(-c4ccc5cc(N(c6ccccc6)c6ccc(-c7cccc8ccccc78)cc6)ccc5c4)c4ccccc43)o2)cc1]

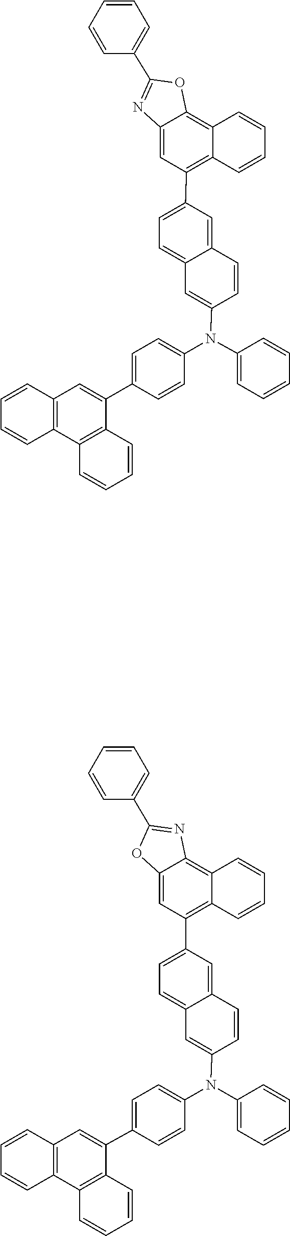 [SMILES: c1ccc(-c2nc3cc(-c4ccc5cc(N(c6ccccc6)c6ccc(-c7cc8ccccc8c8ccccc78)cc6)ccc5c4)c4ccccc4c3o2)cc1 ; c1ccc(-c2nc3c(cc(-c4ccc5cc(N(c6ccccc6)c6ccc(-c7cc8ccccc8c8ccccc78)cc6)ccc5c4)c4ccccc43)o2)cc1]

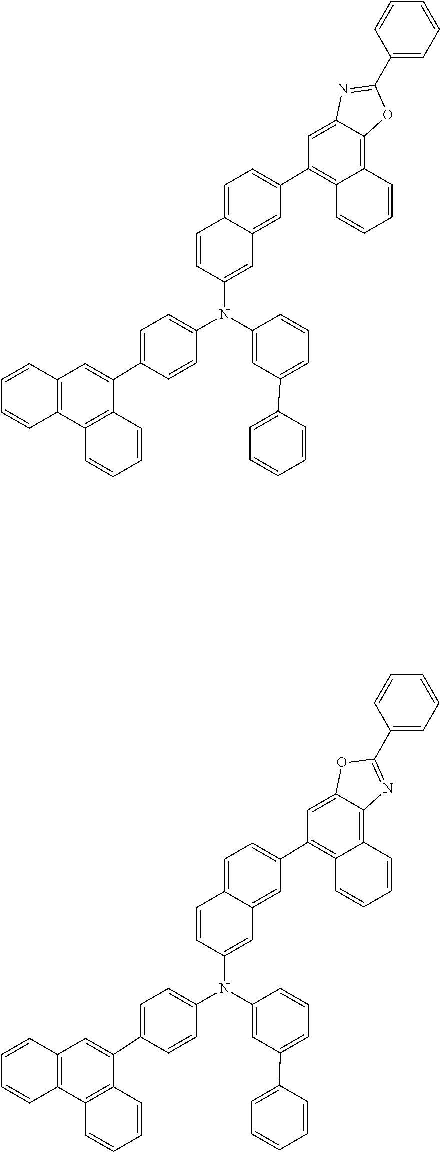 [SMILES: c1ccc(-c2cccc(N(c3ccc(-c4cc5ccccc5c5ccccc45)cc3)c3ccc4ccc(-c5cc6nc(-c7ccccc7)oc6c6ccccc56)cc4c3)c2)cc1 ; c1ccc(-c2cccc(N(c3ccc(-c4cc5ccccc5c5ccccc45)cc3)c3ccc4ccc(-c5cc6oc(-c7ccccc7)nc6c6ccccc56)cc4c3)c2)cc1]

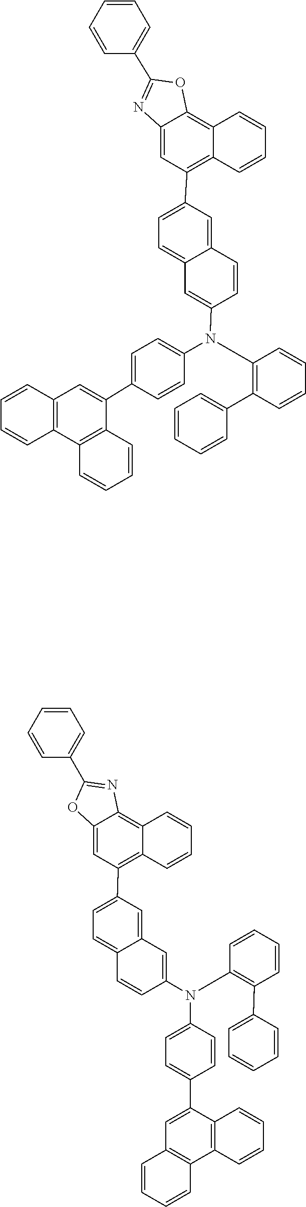 [SMILES: c1ccc(-c2nc3cc(-c4ccc5cc(N(c6ccc(-c7cc8ccccc8c8ccccc78)cc6)c6ccccc6-c6ccccc6)ccc5c4)c4ccccc4c3o2)cc1 ; c1ccc(-c2nc3c(cc(-c4ccc5ccc(N(c6ccc(-c7cc8ccccc8c8ccccc78)cc6)c6ccccc6-c6ccccc6)cc5c4)c4ccccc43)o2)cc1]

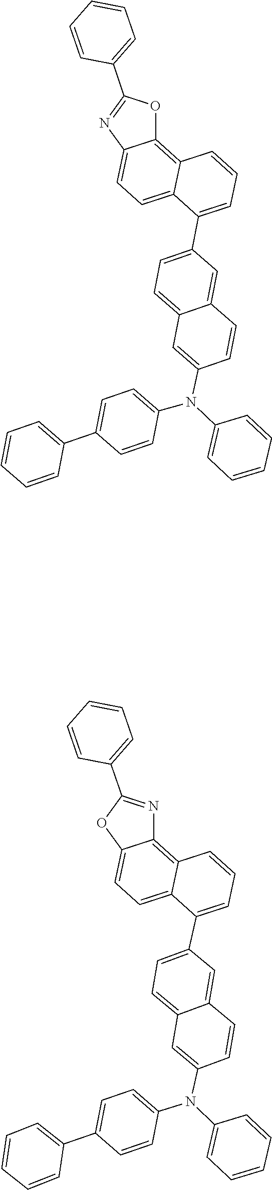 [SMILES: c1ccc(-c2ccc(N(c3ccccc3)c3ccc4cc(-c5cccc6c5ccc5nc(-c7ccccc7)oc56)ccc4c3)cc2)cc1 ; c1ccc(-c2ccc(N(c3ccccc3)c3ccc4cc(-c5cccc6c5ccc5oc(-c7ccccc7)nc56)ccc4c3)cc2)cc1]

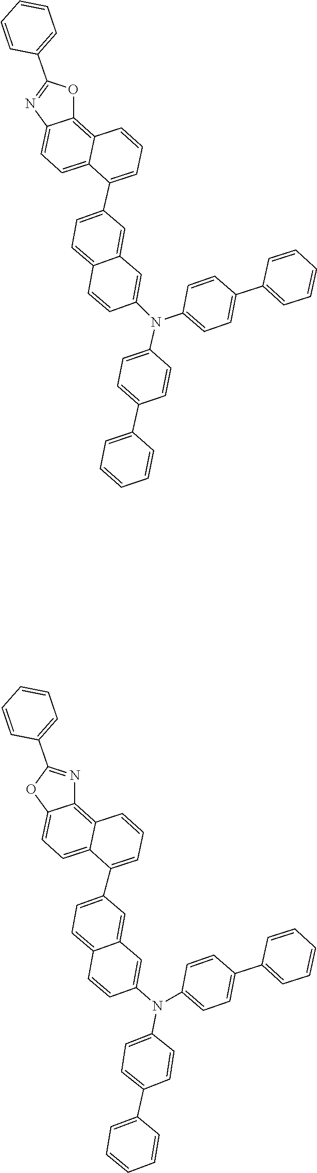 [SMILES: c1ccc(-c2ccc(N(c3ccc(-c4ccccc4)cc3)c3ccc4ccc(-c5cccc6c5ccc5nc(-c7ccccc7)oc56)cc4c3)cc2)cc1 ; c1ccc(-c2ccc(N(c3ccc(-c4ccccc4)cc3)c3ccc4ccc(-c5cccc6c5ccc5oc(-c7ccccc7)nc56)cc4c3)cc2)cc1]

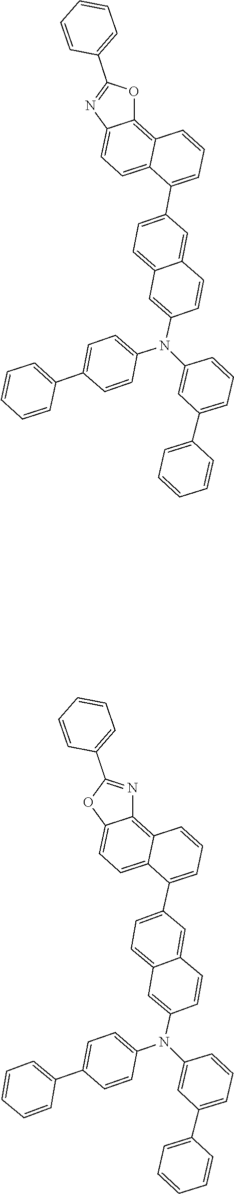 [SMILES: c1ccc(-c2ccc(N(c3cccc(-c4ccccc4)c3)c3ccc4cc(-c5cccc6c5ccc5nc(-c7ccccc7)oc56)ccc4c3)cc2)cc1 ; c1ccc(-c2ccc(N(c3cccc(-c4ccccc4)c3)c3ccc4cc(-c5cccc6c5ccc5oc(-c7ccccc7)nc56)ccc4c3)cc2)cc1]

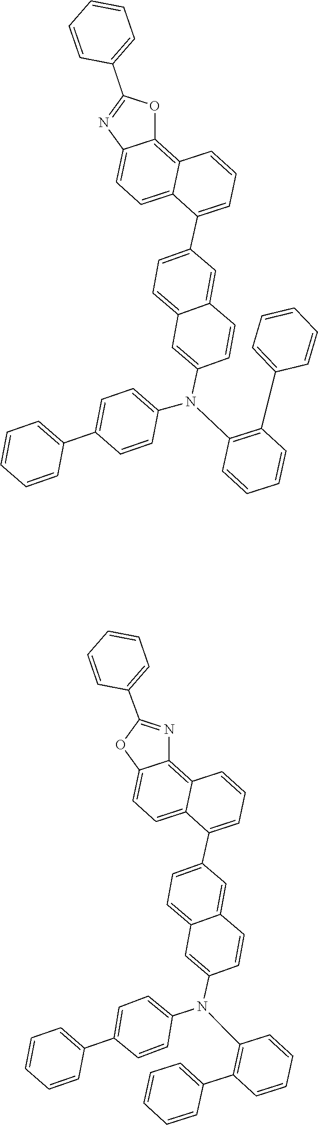 [SMILES: c1ccc(-c2ccc(N(c3ccc4cc(-c5cccc6c5ccc5nc(-c7ccccc7)oc56)ccc4c3)c3ccccc3-c3ccccc3)cc2)cc1 ; c1ccc(-c2ccc(N(c3ccc4cc(-c5cccc6c5ccc5oc(-c7ccccc7)nc56)ccc4c3)c3ccccc3-c3ccccc3)cc2)cc1]

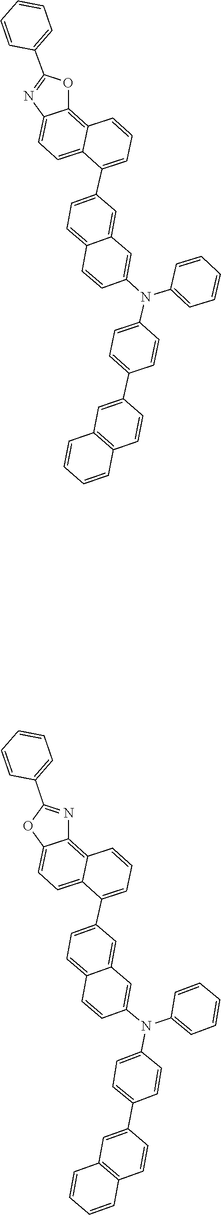 [SMILES: c1ccc(-c2nc3ccc4c(-c5ccc6ccc(N(c7ccccc7)c7ccc(-c8ccc9ccccc9c8)cc7)cc6c5)cccc4c3o2)cc1 ; c1ccc(-c2nc3c(ccc4c(-c5ccc6ccc(N(c7ccccc7)c7ccc(-c8ccc9ccccc9c8)cc7)cc6c5)cccc43)o2)cc1]

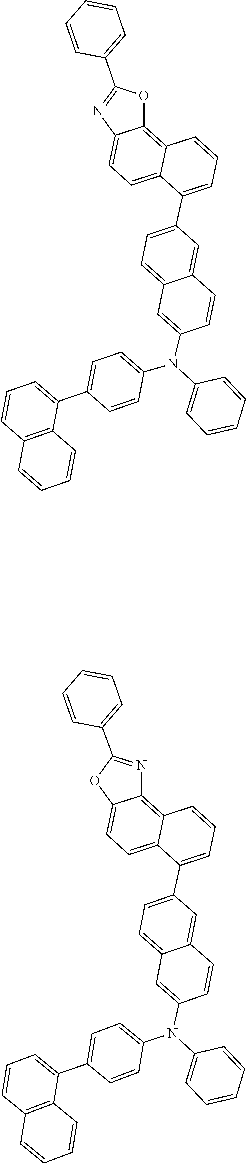 [SMILES: c1ccc(-c2nc3ccc4c(-c5ccc6cc(N(c7ccccc7)c7ccc(-c8cccc9ccccc89)cc7)ccc6c5)cccc4c3o2)cc1 ; c1ccc(-c2nc3c(ccc4c(-c5ccc6cc(N(c7ccccc7)c7ccc(-c8cccc9ccccc89)cc7)ccc6c5)cccc43)o2)cc1]

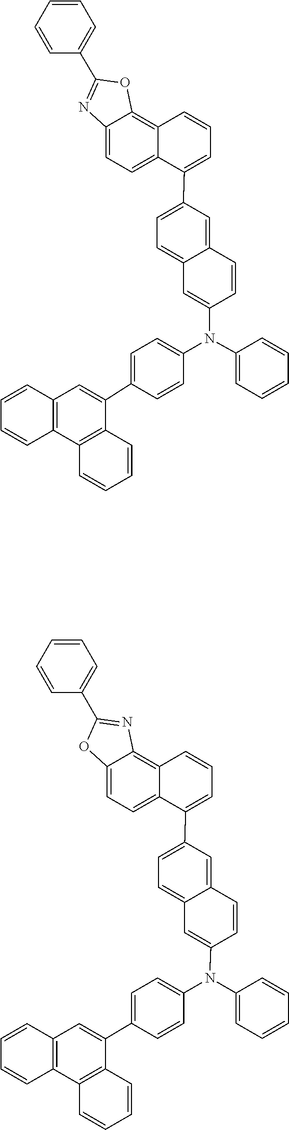 [SMILES: c1ccc(-c2nc3ccc4c(-c5ccc6cc(N(c7ccccc7)c7ccc(-c8cc9ccccc9c9ccccc89)cc7)ccc6c5)cccc4c3o2)cc1 ; c1ccc(-c2nc3c(ccc4c(-c5ccc6cc(N(c7ccccc7)c7ccc(-c8cc9ccccc9c9ccccc89)cc7)ccc6c5)cccc43)o2)cc1]

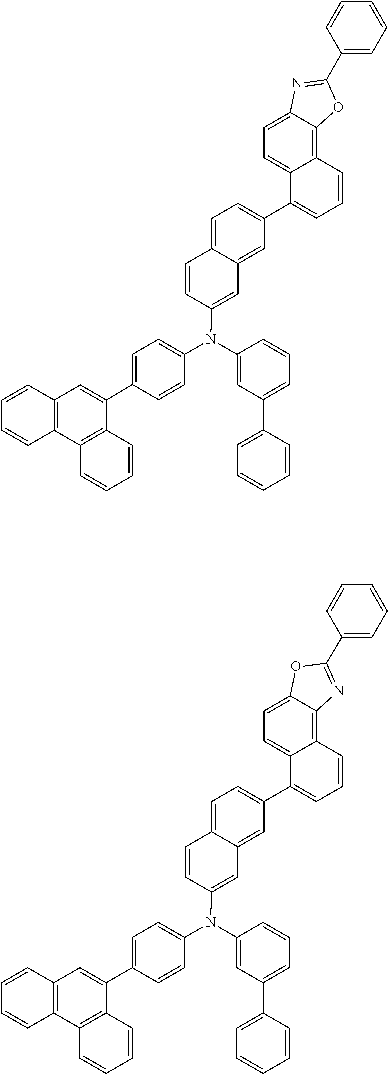 [SMILES: c1ccc(-c2cccc(N(c3ccc(-c4cc5ccccc5c5ccccc45)cc3)c3ccc4ccc(-c5cccc6c5ccc5nc(-c7ccccc7)oc56)cc4c3)c2)cc1 ; c1ccc(-c2cccc(N(c3ccc(-c4cc5ccccc5c5ccccc45)cc3)c3ccc4ccc(-c5cccc6c5ccc5oc(-c7ccccc7)nc56)cc4c3)c2)cc1]

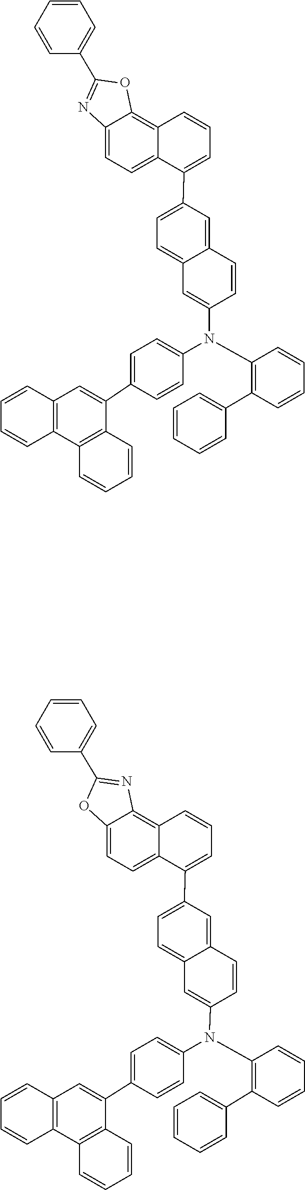 [SMILES: c1ccc(-c2nc3ccc4c(-c5ccc6cc(N(c7ccc(-c8cc9ccccc9c9ccccc89)cc7)c7ccccc7-c7ccccc7)ccc6c5)cccc4c3o2)cc1 ; c1ccc(-c2nc3c(ccc4c(-c5ccc6cc(N(c7ccc(-c8cc9ccccc9c9ccccc89)cc7)c7ccccc7-c7ccccc7)ccc6c5)cccc43)o2)cc1]

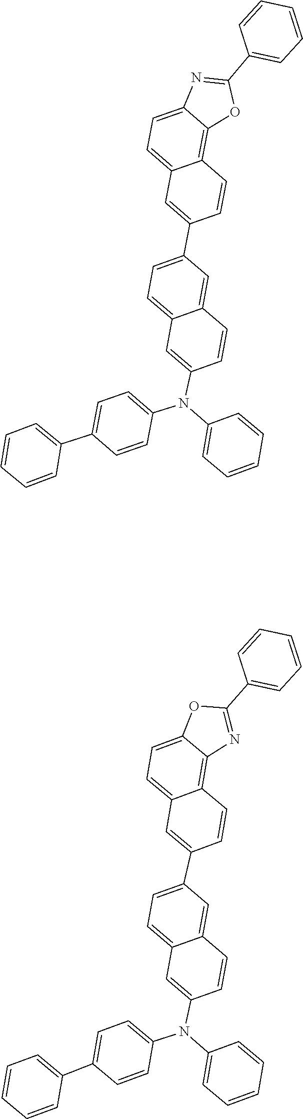 [SMILES: c1ccc(-c2ccc(N(c3ccccc3)c3ccc4cc(-c5ccc6c(ccc7nc(-c8ccccc8)oc76)c5)ccc4c3)cc2)cc1 ; c1ccc(-c2ccc(N(c3ccccc3)c3ccc4cc(-c5ccc6c(ccc7oc(-c8ccccc8)nc76)c5)ccc4c3)cc2)cc1]

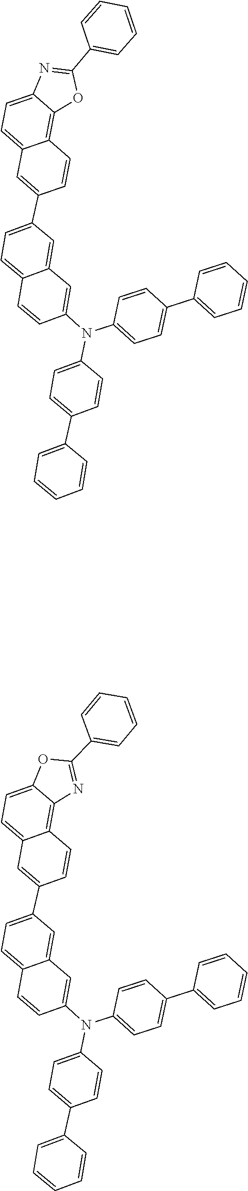 [SMILES: c1ccc(-c2ccc(N(c3ccc(-c4ccccc4)cc3)c3ccc4ccc(-c5ccc6c(ccc7nc(-c8ccccc8)oc76)c5)cc4c3)cc2)cc1 ; c1ccc(-c2ccc(N(c3ccc(-c4ccccc4)cc3)c3ccc4ccc(-c5ccc6c(ccc7oc(-c8ccccc8)nc76)c5)cc4c3)cc2)cc1]

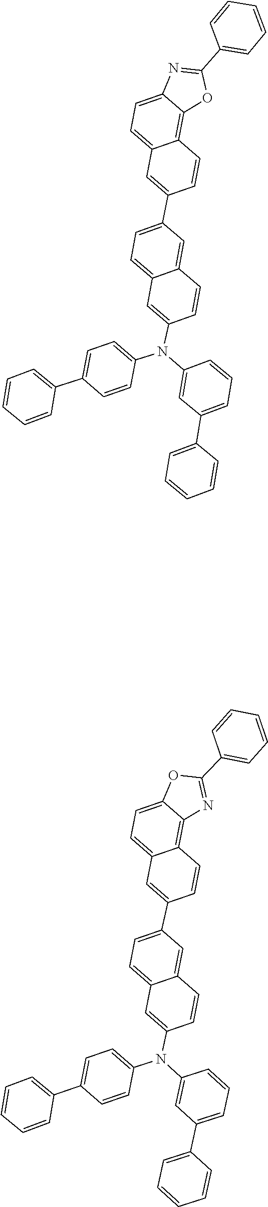 [SMILES: c1ccc(-c2ccc(N(c3cccc(-c4ccccc4)c3)c3ccc4cc(-c5ccc6c(ccc7nc(-c8ccccc8)oc76)c5)ccc4c3)cc2)cc1 ; c1ccc(-c2ccc(N(c3cccc(-c4ccccc4)c3)c3ccc4cc(-c5ccc6c(ccc7oc(-c8ccccc8)nc76)c5)ccc4c3)cc2)cc1]

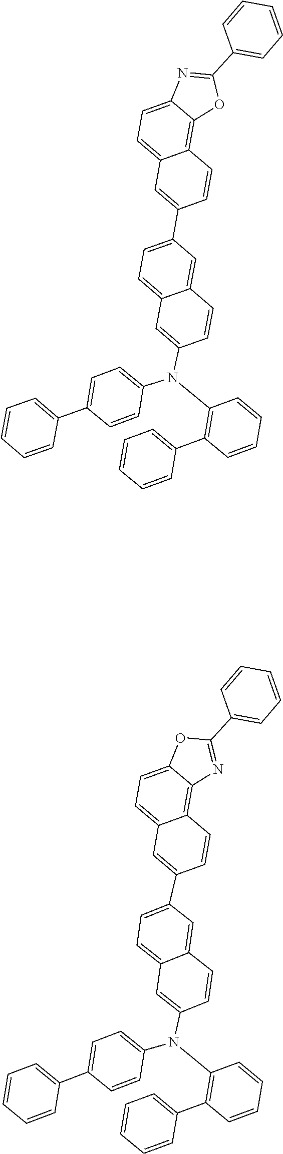

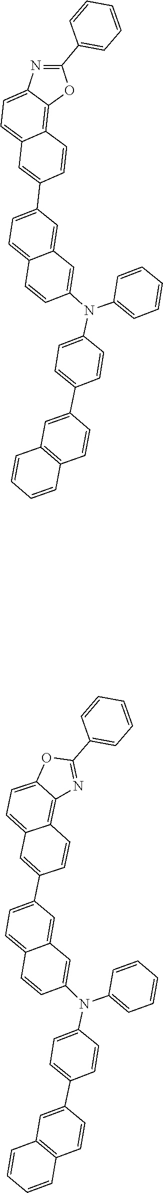 [SMILES: c1ccc(-c2nc3ccc4cc(-c5ccc6ccc(N(c7ccccc7)c7ccc(-c8ccc9ccccc9c8)cc7)cc6c5)ccc4c3o2)cc1 ; c1ccc(-c2nc3c(ccc4cc(-c5ccc6ccc(N(c7ccccc7)c7ccc(-c8ccc9ccccc9c8)cc7)cc6c5)ccc43)o2)cc1]

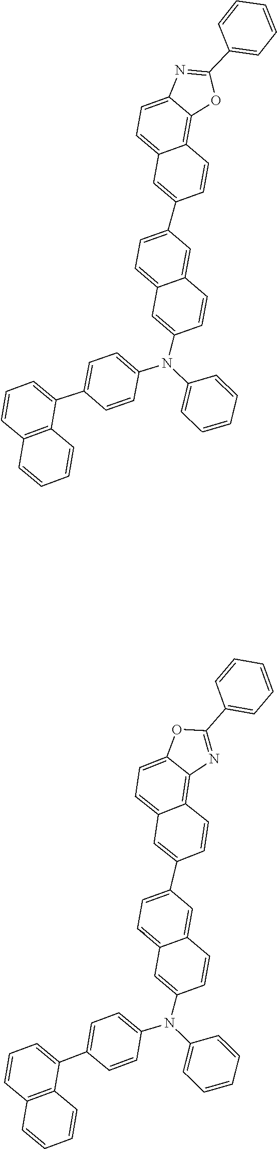

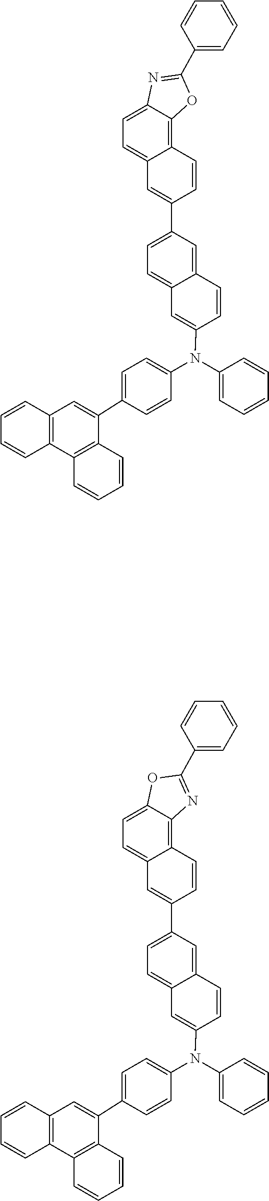 [SMILES: c1ccc(-c2nc3ccc4cc(-c5ccc6cc(N(c7ccccc7)c7ccc(-c8cc9ccccc9c9ccccc89)cc7)ccc6c5)ccc4c3o2)cc1 ; c1ccc(-c2nc3c(ccc4cc(-c5ccc6cc(N(c7ccccc7)c7ccc(-c8cc9ccccc9c9ccccc89)cc7)ccc6c5)ccc43)o2)cc1]

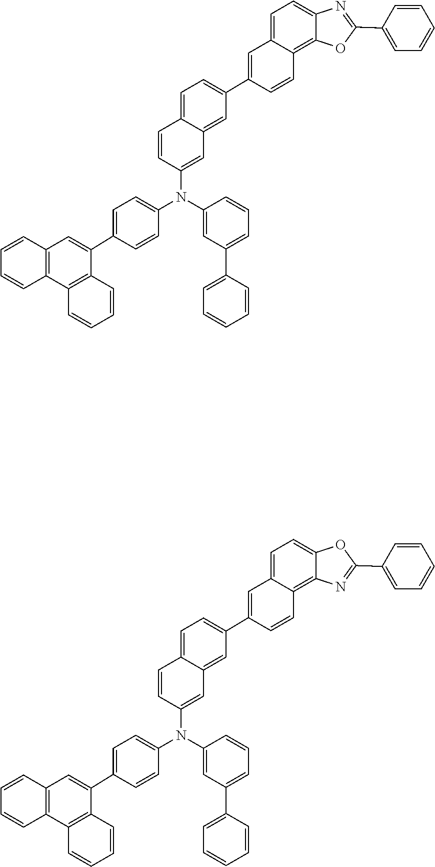 [SMILES: c1ccc(-c2cccc(N(c3ccc(-c4cc5ccccc5c5ccccc45)cc3)c3ccc4ccc(-c5ccc6c(ccc7nc(-c8ccccc8)oc76)c5)cc4c3)c2)cc1 ; c1ccc(-c2cccc(N(c3ccc(-c4cc5ccccc5c5ccccc45)cc3)c3ccc4ccc(-c5ccc6c(ccc7oc(-c8ccccc8)nc76)c5)cc4c3)c2)cc1]

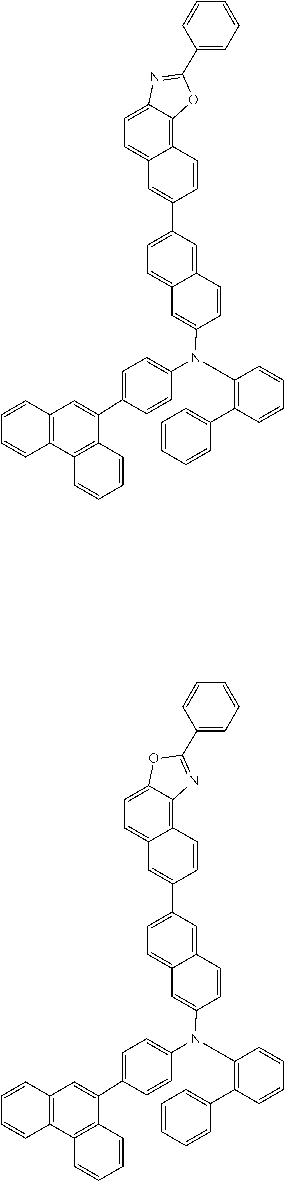 [SMILES: c1ccc(-c2nc3ccc4cc(-c5ccc6cc(N(c7ccc(-c8cc9ccccc9c9ccccc89)cc7)c7ccccc7-c7ccccc7)ccc6c5)ccc4c3o2)cc1 ; c1ccc(-c2nc3c(ccc4cc(-c5ccc6cc(N(c7ccc(-c8cc9ccccc9c9ccccc89)cc7)c7ccccc7-c7ccccc7)ccc6c5)ccc43)o2)cc1]

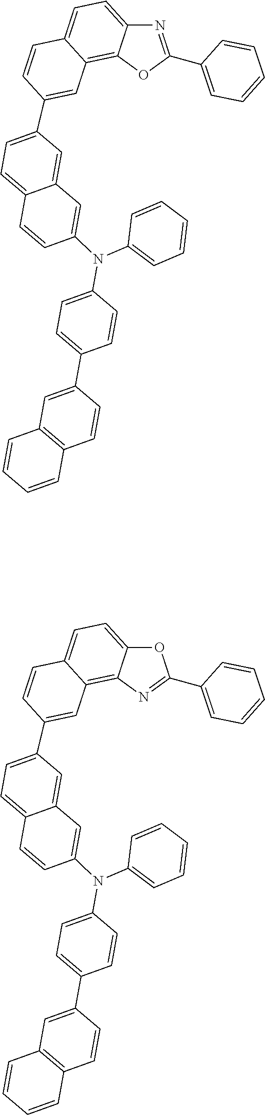 [SMILES: c1ccc(-c2nc3ccc4ccc(-c5ccc6ccc(N(c7ccccc7)c7ccc(-c8ccc9ccccc9c8)cc7)cc6c5)cc4c3o2)cc1 ; c1ccc(-c2nc3c(ccc4ccc(-c5ccc6ccc(N(c7ccccc7)c7ccc(-c8ccc9ccccc9c8)cc7)cc6c5)cc43)o2)cc1]

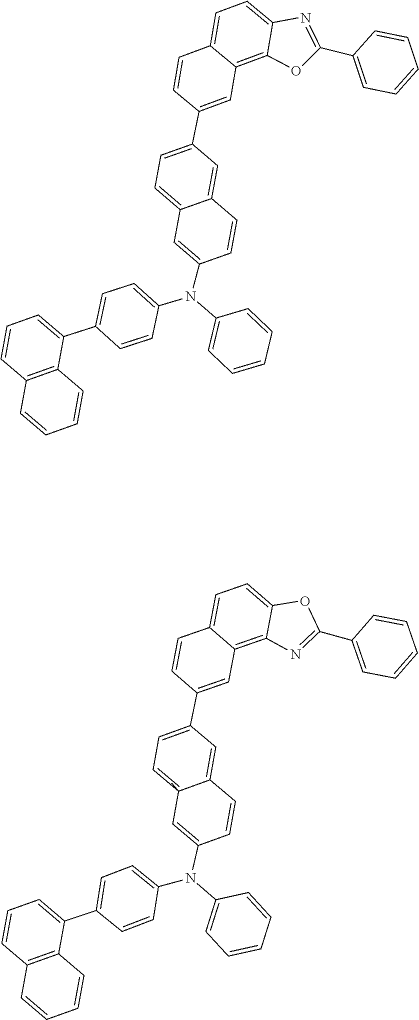

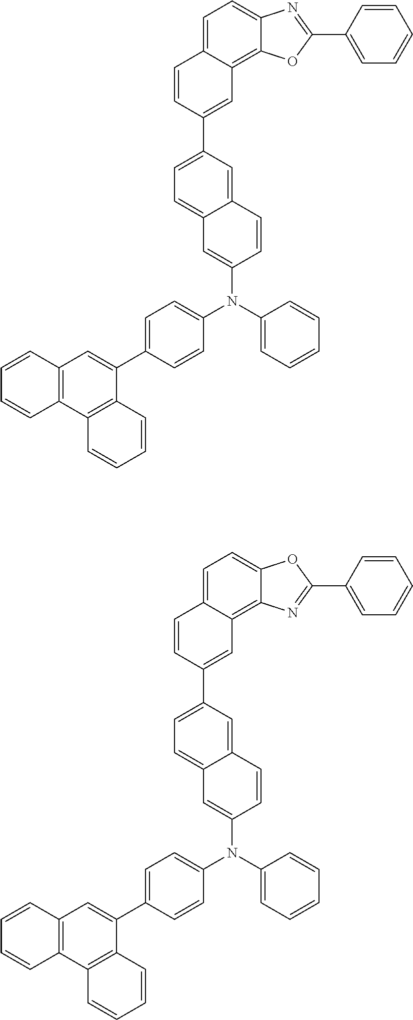 [SMILES: c1ccc(-c2nc3ccc4ccc(-c5ccc6cc(N(c7ccccc7)c7ccc(-c8cc9ccccc9c9ccccc89)cc7)ccc6c5)cc4c3o2)cc1 ; c1ccc(-c2nc3c(ccc4ccc(-c5ccc6cc(N(c7ccccc7)c7ccc(-c8cc9ccccc9c9ccccc89)cc7)ccc6c5)cc43)o2)cc1]

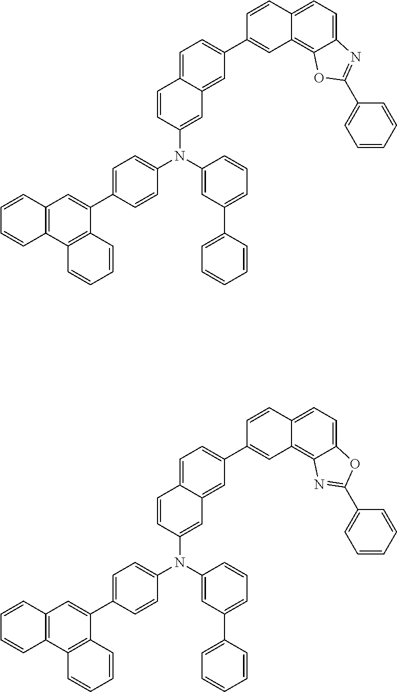 [SMILES: c1ccc(-c2cccc(N(c3ccc(-c4cc5ccccc5c5ccccc45)cc3)c3ccc4ccc(-c5ccc6ccc7nc(-c8ccccc8)oc7c6c5)cc4c3)c2)cc1 ; c1ccc(-c2cccc(N(c3ccc(-c4cc5ccccc5c5ccccc45)cc3)c3ccc4ccc(-c5ccc6ccc7oc(-c8ccccc8)nc7c6c5)cc4c3)c2)cc1]

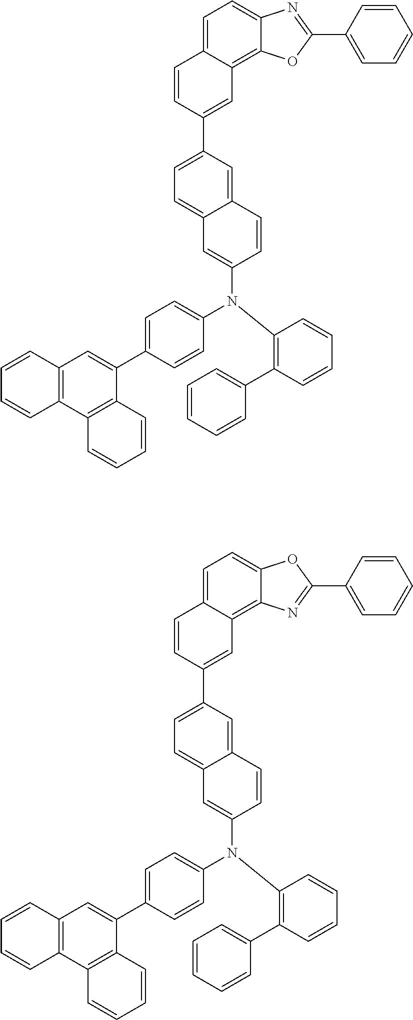 [SMILES: c1ccc(-c2nc3ccc4ccc(-c5ccc6cc(N(c7ccc(-c8cc9ccccc9c9ccccc89)cc7)c7ccccc7-c7ccccc7)ccc6c5)cc4c3o2)cc1 ; c1ccc(-c2nc3c(ccc4ccc(-c5ccc6cc(N(c7ccc(-c8cc9ccccc9c9ccccc89)cc7)c7ccccc7-c7ccccc7)ccc6c5)cc43)o2)cc1]

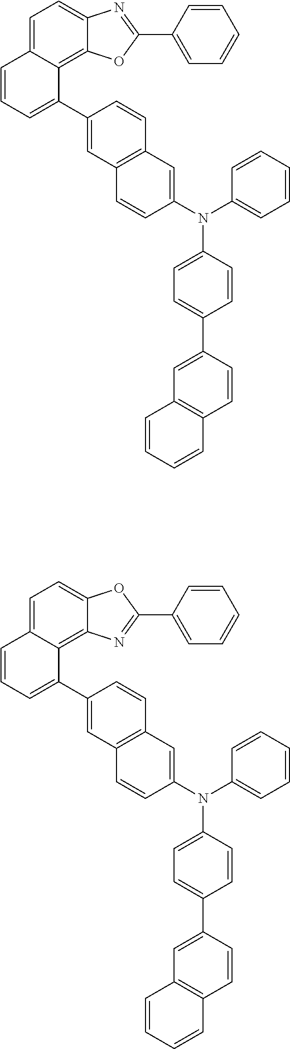 [SMILES: c1ccc(-c2nc3ccc4cccc(-c5ccc6cc(N(c7ccccc7)c7ccc(-c8ccc9ccccc9c8)cc7)ccc6c5)c4c3o2)cc1 ; c1ccc(-c2nc3c(ccc4cccc(-c5ccc6cc(N(c7ccccc7)c7ccc(-c8ccc9ccccc9c8)cc7)ccc6c5)c43)o2)cc1]

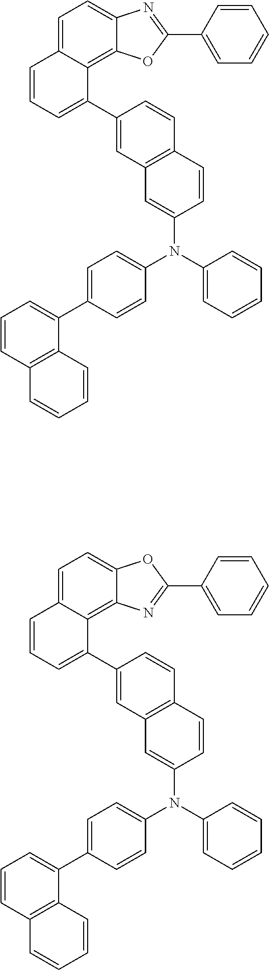 [SMILES: c1ccc(-c2nc3ccc4cccc(-c5ccc6ccc(N(c7ccccc7)c7ccc(-c8cccc9ccccc89)cc7)cc6c5)c4c3o2)cc1 ; c1ccc(-c2nc3c(ccc4cccc(-c5ccc6ccc(N(c7ccccc7)c7ccc(-c8cccc9ccccc89)cc7)cc6c5)c43)o2)cc1]

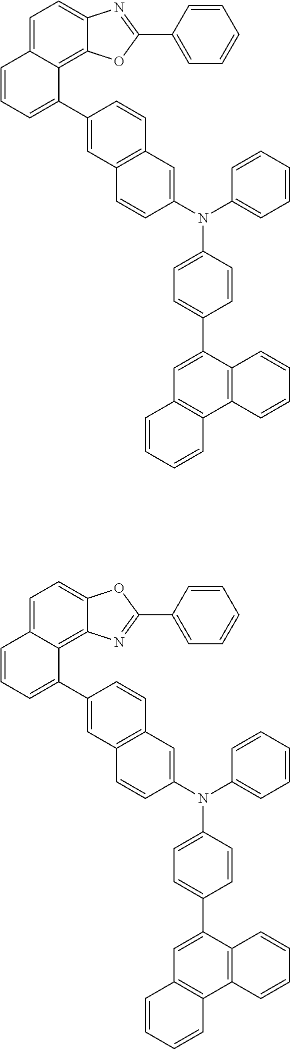 [SMILES: c1ccc(-c2nc3ccc4cccc(-c5ccc6cc(N(c7ccccc7)c7ccc(-c8cc9ccccc9c9ccccc89)cc7)ccc6c5)c4c3o2)cc1 ; c1ccc(-c2nc3c(ccc4cccc(-c5ccc6cc(N(c7ccccc7)c7ccc(-c8cc9ccccc9c9ccccc89)cc7)ccc6c5)c43)o2)cc1]

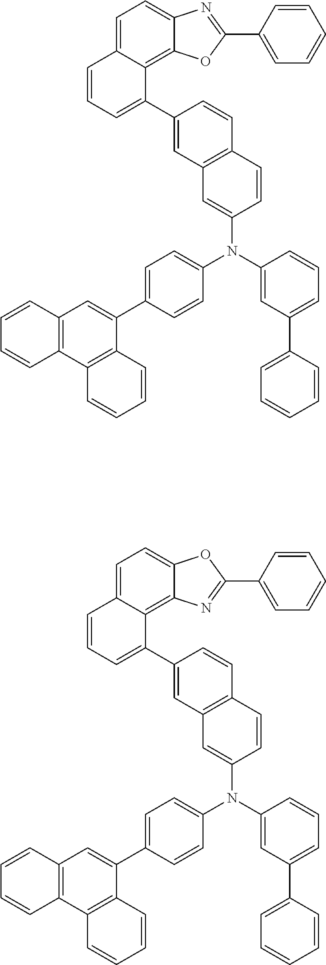 [SMILES: c1ccc(-c2cccc(N(c3ccc(-c4cc5ccccc5c5ccccc45)cc3)c3ccc4ccc(-c5cccc6ccc7nc(-c8ccccc8)oc7c56)cc4c3)c2)cc1 ; c1ccc(-c2cccc(N(c3ccc(-c4cc5ccccc5c5ccccc45)cc3)c3ccc4ccc(-c5cccc6ccc7oc(-c8ccccc8)nc7c56)cc4c3)c2)cc1]

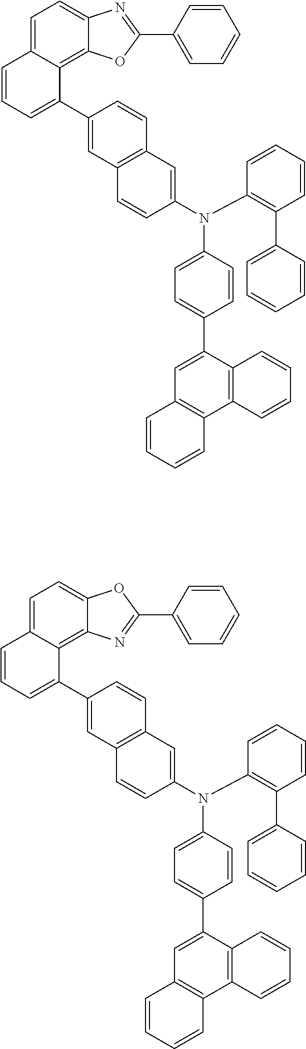 [SMILES: c1ccc(-c2nc3ccc4cccc(-c5ccc6cc(N(c7ccc(-c8cc9ccccc9c9ccccc89)cc7)c7ccccc7-c7ccccc7)ccc6c5)c4c3o2)cc1 ; c1ccc(-c2nc3c(ccc4cccc(-c5ccc6cc(N(c7ccc(-c8cc9ccccc9c9ccccc89)cc7)c7ccccc7-c7ccccc7)ccc6c5)c43)o2)cc1]

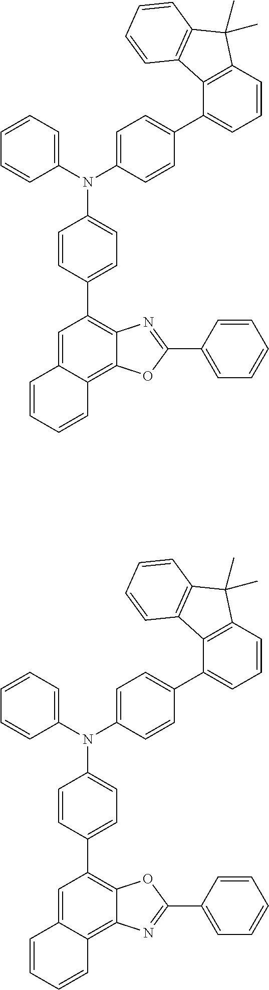 [SMILES: CC1(C)c2ccccc2-c2c(-c3ccc(N(c4ccccc4)c4ccc(-c5cc6ccccc6c6oc(-c7ccccc7)nc56)cc4)cc3)cccc21 ; CC1(C)c2ccccc2-c2c(-c3ccc(N(c4ccccc4)c4ccc(-c5cc6ccccc6c6nc(-c7ccccc7)oc56)cc4)cc3)cccc21]

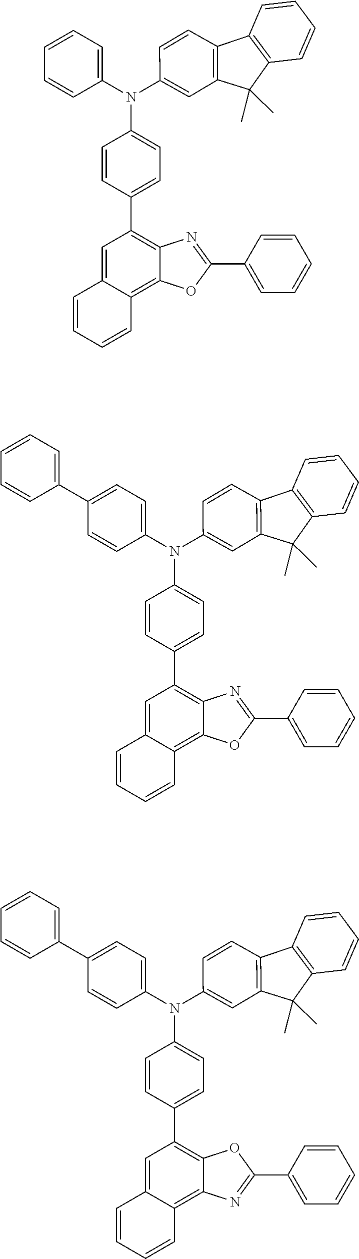 [SMILES: CC1(C)c2ccccc2-c2ccc(N(c3ccccc3)c3ccc(-c4cc5ccccc5c5oc(-c6ccccc6)nc45)cc3)cc21 ; CC1(C)c2ccccc2-c2ccc(N(c3ccc(-c4ccccc4)cc3)c3ccc(-c4cc5ccccc5c5oc(-c6ccccc6)nc45)cc3)cc21 ; CC1(C)c2ccccc2-c2ccc(N(c3ccc(-c4ccccc4)cc3)c3ccc(-c4cc5ccccc5c5nc(-c6ccccc6)oc45)cc3)cc21]

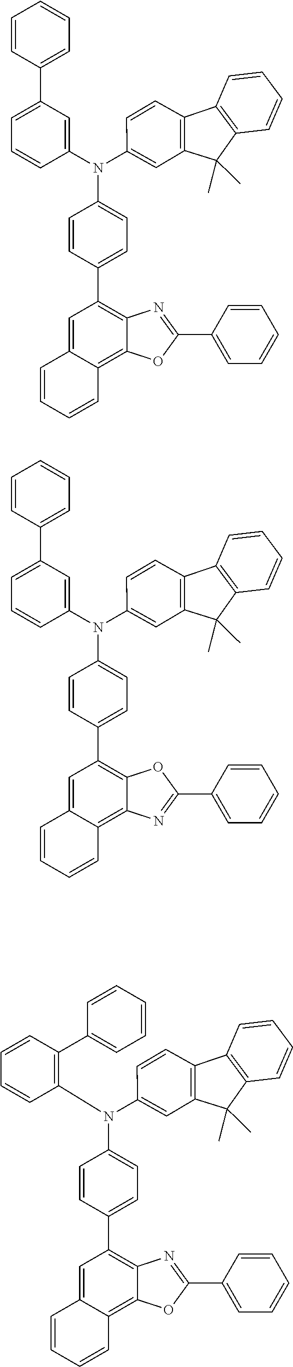 [SMILES: CC1(C)c2ccccc2-c2ccc(N(c3ccc(-c4cc5ccccc5c5oc(-c6ccccc6)nc45)cc3)c3cccc(-c4ccccc4)c3)cc21 ; CC1(C)c2ccccc2-c2ccc(N(c3ccc(-c4cc5ccccc5c5nc(-c6ccccc6)oc45)cc3)c3cccc(-c4ccccc4)c3)cc21 ; CC1(C)c2ccccc2-c2ccc(N(c3ccc(-c4cc5ccccc5c5oc(-c6ccccc6)nc45)cc3)c3ccccc3-c3ccccc3)cc21]

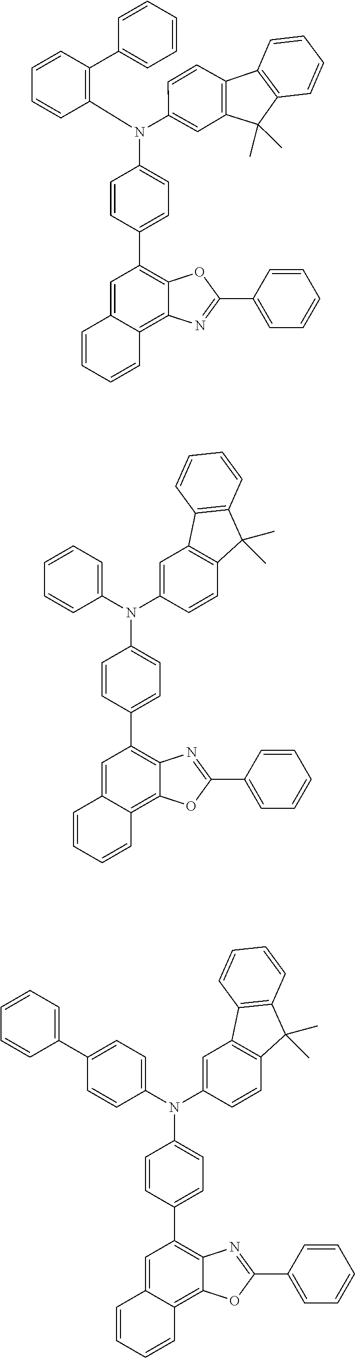 [SMILES: CC1(C)c2ccccc2-c2ccc(N(c3ccc(-c4cc5ccccc5c5nc(-c6ccccc6)oc45)cc3)c3ccccc3-c3ccccc3)cc21 ; CC1(C)c2ccccc2-c2cc(N(c3ccccc3)c3ccc(-c4cc5ccccc5c5oc(-c6ccccc6)nc45)cc3)ccc21 ; CC1(C)c2ccccc2-c2cc(N(c3ccc(-c4ccccc4)cc3)c3ccc(-c4cc5ccccc5c5oc(-c6ccccc6)nc45)cc3)ccc21]

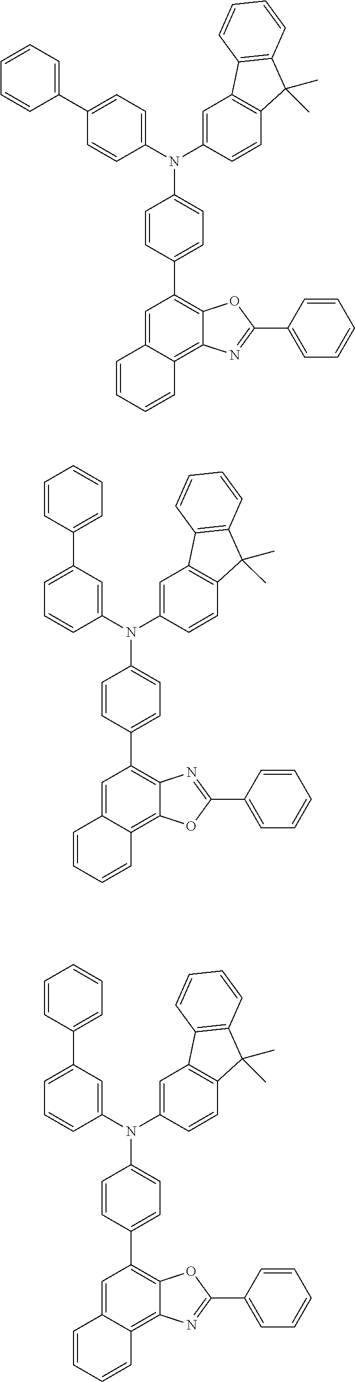 [SMILES: CC1(C)c2ccccc2-c2cc(N(c3ccc(-c4ccccc4)cc3)c3ccc(-c4cc5ccccc5c5nc(-c6ccccc6)oc45)cc3)ccc21 ; CC1(C)c2ccccc2-c2cc(N(c3ccc(-c4cc5ccccc5c5oc(-c6ccccc6)nc45)cc3)c3cccc(-c4ccccc4)c3)ccc21 ; CC1(C)c2ccccc2-c2cc(N(c3ccc(-c4cc5ccccc5c5nc(-c6ccccc6)oc45)cc3)c3cccc(-c4ccccc4)c3)ccc21]

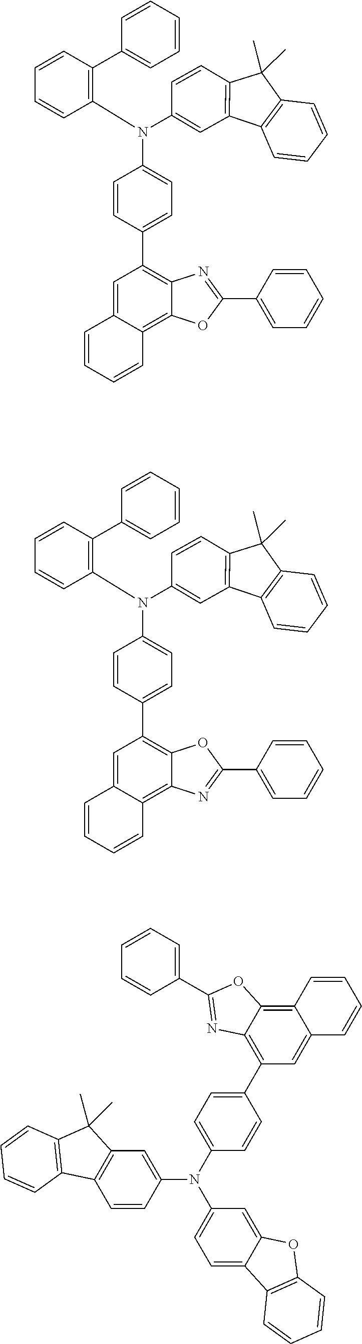 [SMILES: CC1(C)c2ccccc2-c2cc(N(c3ccc(-c4cc5ccccc5c5oc(-c6ccccc6)nc45)cc3)c3ccccc3-c3ccccc3)ccc21 ; CC1(C)c2ccccc2-c2cc(N(c3ccc(-c4cc5ccccc5c5nc(-c6ccccc6)oc45)cc3)c3ccccc3-c3ccccc3)ccc21 ; CC1(C)c2ccccc2-c2ccc(N(c3ccc(-c4cc5ccccc5c5oc(-c6ccccc6)nc45)cc3)c3ccc4c(c3)oc3ccccc34)cc21]

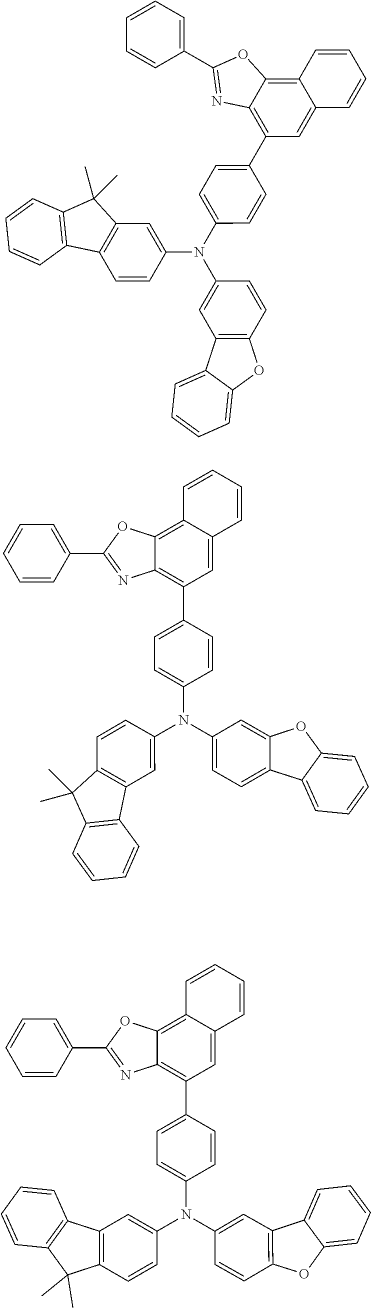

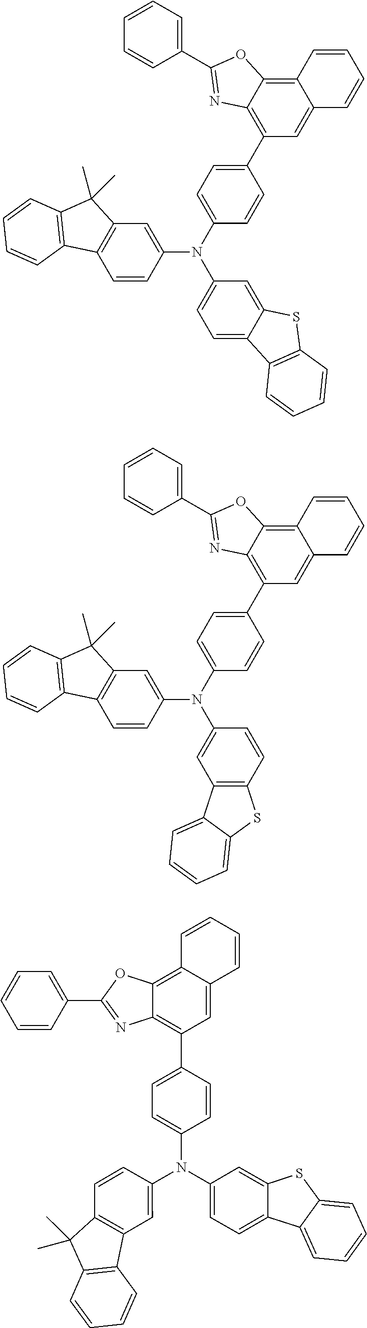

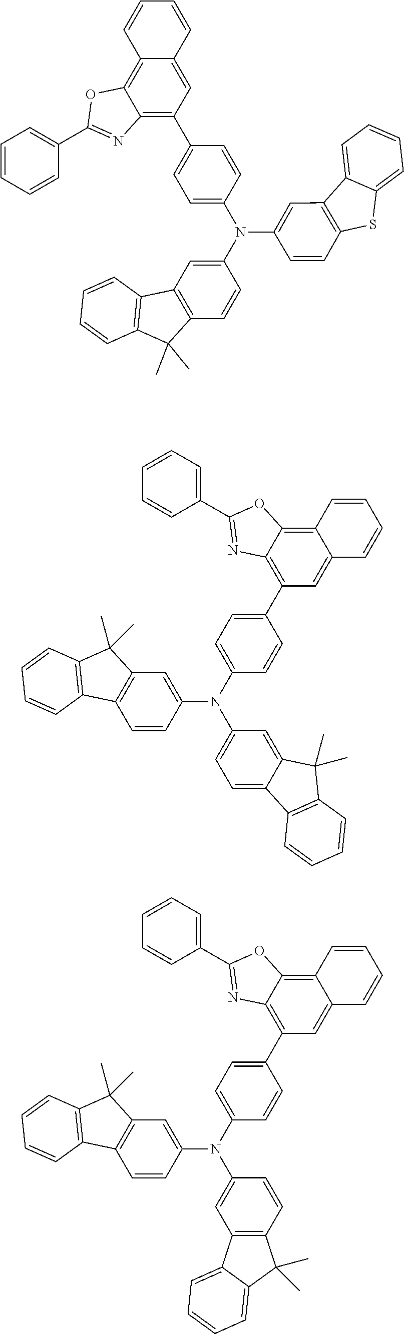 [SMILES: CC1(C)c2ccccc2-c2cc(N(c3ccc(-c4cc5ccccc5c5oc(-c6ccccc6)nc45)cc3)c3ccc4sc5ccccc5c4c3)ccc21 ; CC1(C)c2ccccc2-c2ccc(N(c3ccc(-c4cc5ccccc5c5oc(-c6ccccc6)nc45)cc3)c3ccc4c(c3)C(C)(C)c3ccccc3-4)cc21 ; CC1(C)c2ccccc2-c2cc(N(c3ccc(-c4cc5ccccc5c5oc(-c6ccccc6)nc45)cc3)c3ccc4c(c3)C(C)(C)c3ccccc3-4)ccc21]

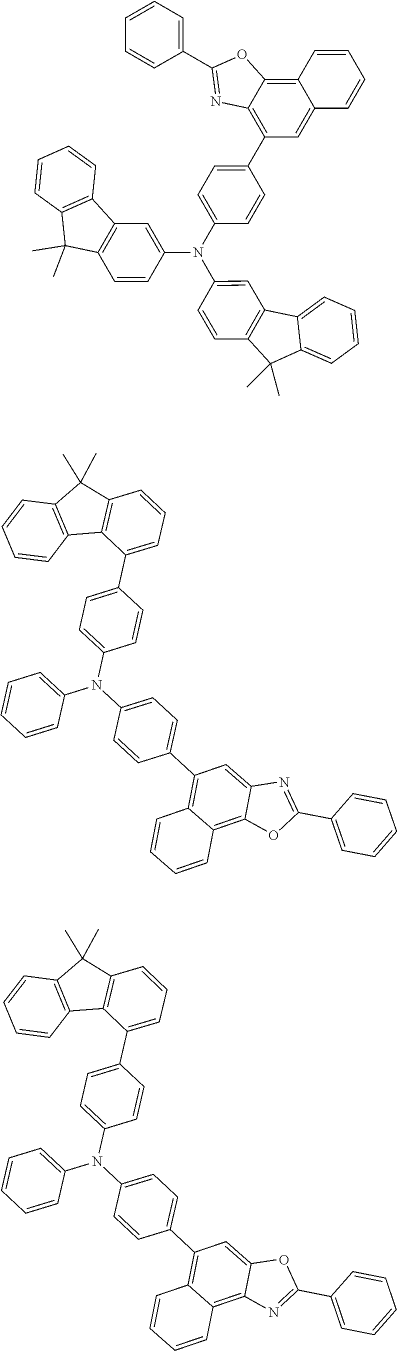 [SMILES: CC1(C)c2ccccc2-c2cc(N(c3ccc(-c4cc5ccccc5c5oc(-c6ccccc6)nc45)cc3)c3ccc4c(c3)-c3ccccc3C4(C)C)ccc21 ; CC1(C)c2ccccc2-c2c(-c3ccc(N(c4ccccc4)c4ccc(-c5cc6nc(-c7ccccc7)oc6c6ccccc56)cc4)cc3)cccc21 ; CC1(C)c2ccccc2-c2c(-c3ccc(N(c4ccccc4)c4ccc(-c5cc6oc(-c7ccccc7)nc6c6ccccc56)cc4)cc3)cccc21]

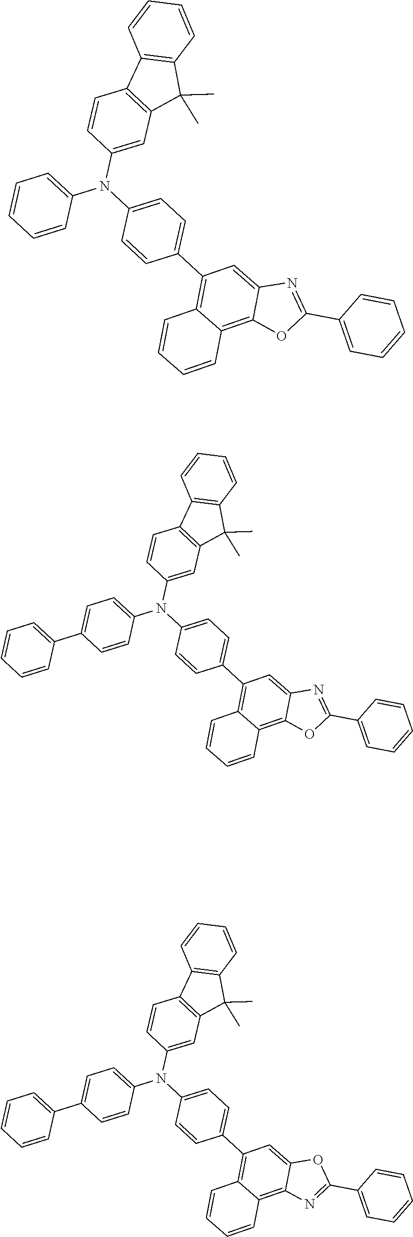 [SMILES: CC1(C)c2ccccc2-c2ccc(N(c3ccccc3)c3ccc(-c4cc5nc(-c6ccccc6)oc5c5ccccc45)cc3)cc21 ; CC1(C)c2ccccc2-c2ccc(N(c3ccc(-c4ccccc4)cc3)c3ccc(-c4cc5nc(-c6ccccc6)oc5c5ccccc45)cc3)cc21 ; CC1(C)c2ccccc2-c2ccc(N(c3ccc(-c4ccccc4)cc3)c3ccc(-c4cc5oc(-c6ccccc6)nc5c5ccccc45)cc3)cc21]

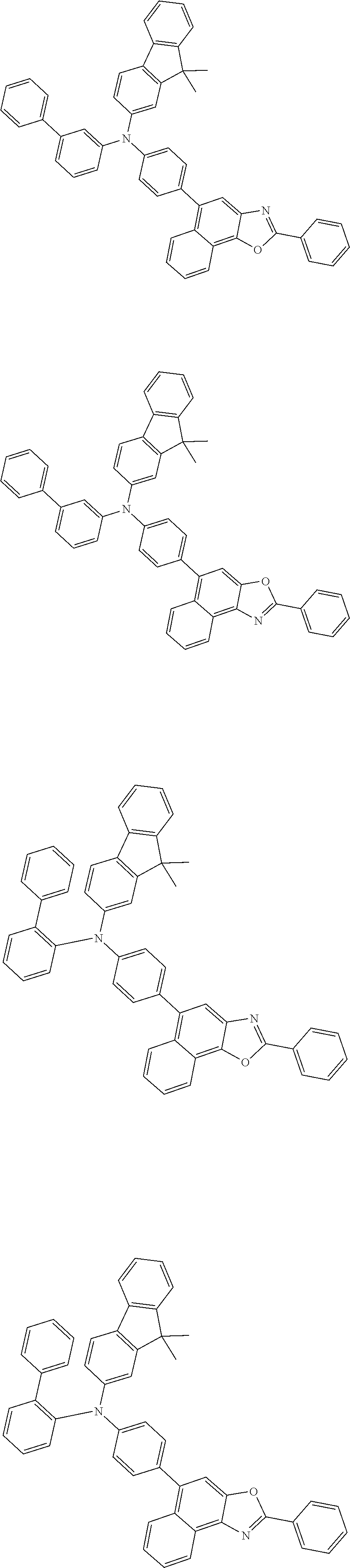 [SMILES: CC1(C)c2ccccc2-c2ccc(N(c3ccc(-c4cc5nc(-c6ccccc6)oc5c5ccccc45)cc3)c3cccc(-c4ccccc4)c3)cc21 ; CC1(C)c2ccccc2-c2ccc(N(c3ccc(-c4cc5oc(-c6ccccc6)nc5c5ccccc45)cc3)c3cccc(-c4ccccc4)c3)cc21 ; CC1(C)c2ccccc2-c2ccc(N(c3ccc(-c4cc5nc(-c6ccccc6)oc5c5ccccc45)cc3)c3ccccc3-c3ccccc3)cc21 ; CC1(C)c2ccccc2-c2ccc(N(c3ccc(-c4cc5oc(-c6ccccc6)nc5c5ccccc45)cc3)c3ccccc3-c3ccccc3)cc21]

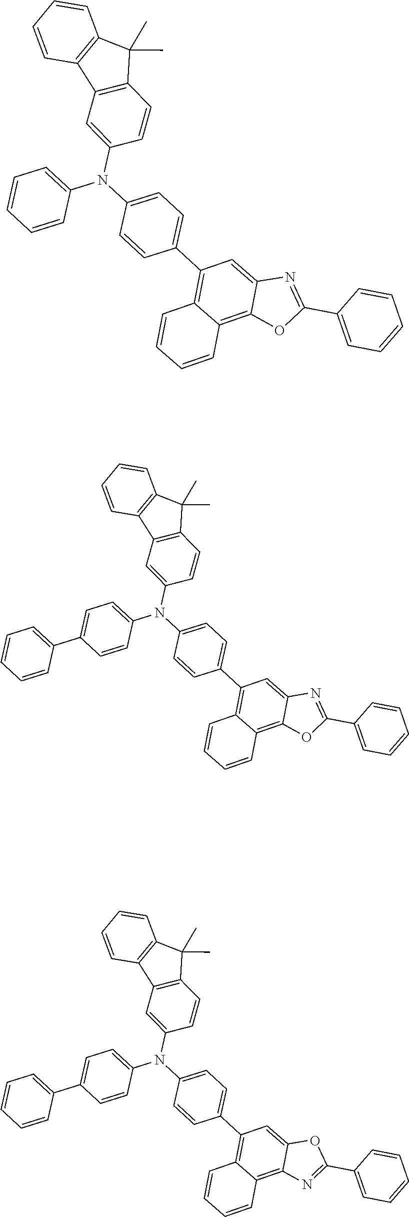 [SMILES: CC1(C)c2ccccc2-c2cc(N(c3ccccc3)c3ccc(-c4cc5nc(-c6ccccc6)oc5c5ccccc45)cc3)ccc21 ; CC1(C)c2ccccc2-c2cc(N(c3ccc(-c4ccccc4)cc3)c3ccc(-c4cc5nc(-c6ccccc6)oc5c5ccccc45)cc3)ccc21 ; CC1(C)c2ccccc2-c2cc(N(c3ccc(-c4ccccc4)cc3)c3ccc(-c4cc5oc(-c6ccccc6)nc5c5ccccc45)cc3)ccc21]

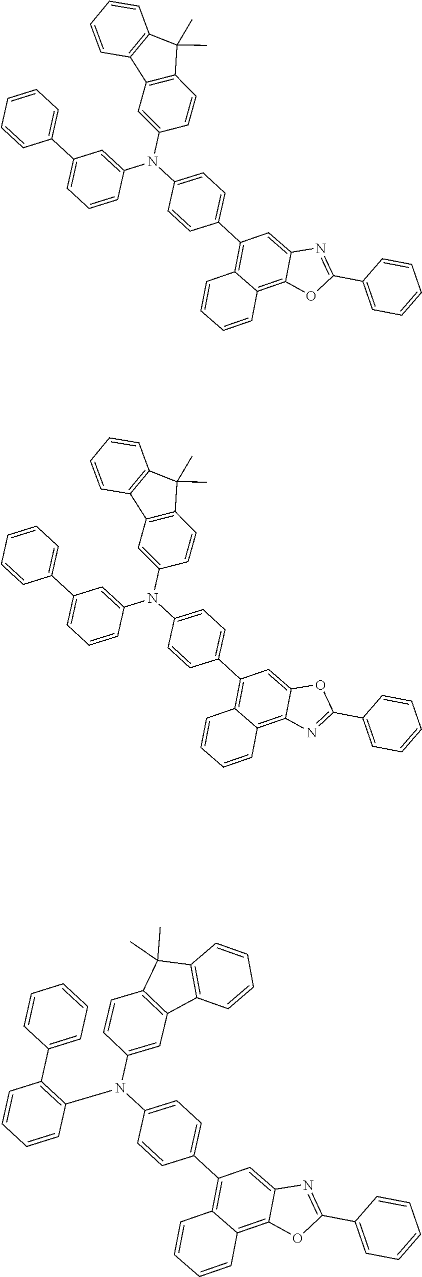 [SMILES: CC1(C)c2ccccc2-c2cc(N(c3ccc(-c4cc5nc(-c6ccccc6)oc5c5ccccc45)cc3)c3cccc(-c4ccccc4)c3)ccc21 ; CC1(C)c2ccccc2-c2cc(N(c3ccc(-c4cc5oc(-c6ccccc6)nc5c5ccccc45)cc3)c3cccc(-c4ccccc4)c3)ccc21 ; CC1(C)c2ccccc2-c2cc(N(c3ccc(-c4cc5nc(-c6ccccc6)oc5c5ccccc45)cc3)c3ccccc3-c3ccccc3)ccc21]

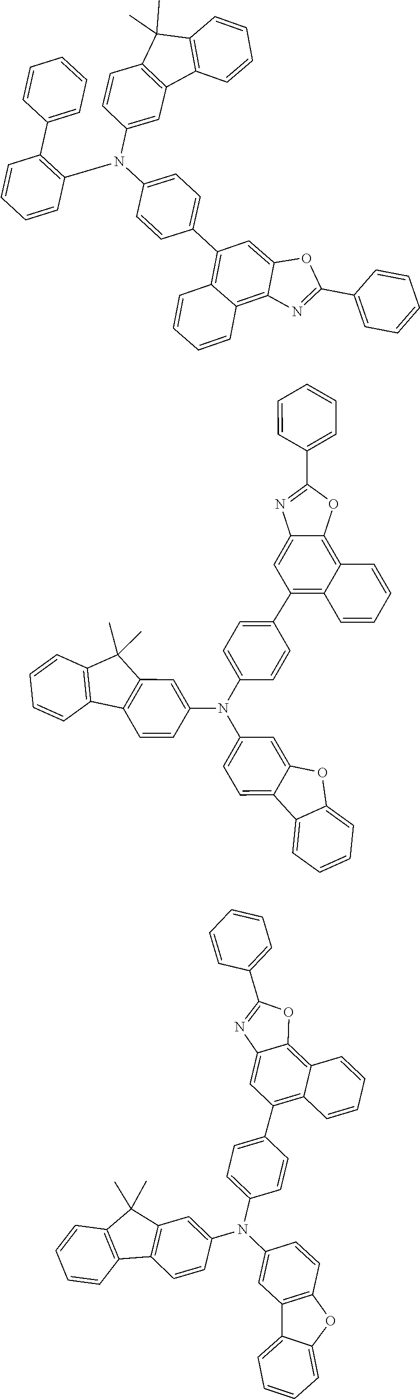 [SMILES: CC1(C)c2ccccc2-c2cc(N(c3ccc(-c4cc5oc(-c6ccccc6)nc5c5ccccc45)cc3)c3ccccc3-c3ccccc3)ccc21 ; CC1(C)c2ccccc2-c2ccc(N(c3ccc(-c4cc5nc(-c6ccccc6)oc5c5ccccc45)cc3)c3ccc4c(c3)oc3ccccc34)cc21 ; CC1(C)c2ccccc2-c2ccc(N(c3ccc(-c4cc5nc(-c6ccccc6)oc5c5ccccc45)cc3)c3ccc4oc5ccccc5c4c3)cc21]

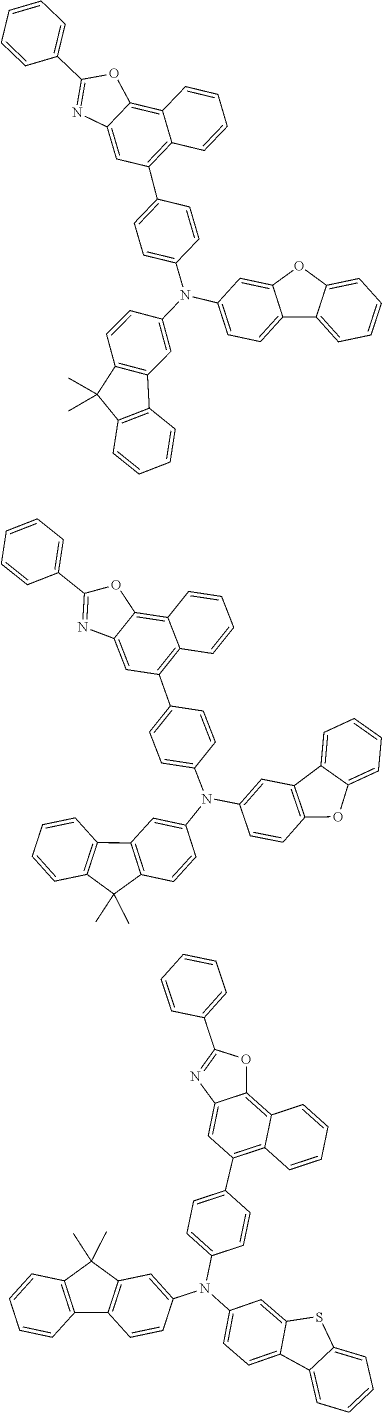 [SMILES: CC1(C)c2ccccc2-c2cc(N(c3ccc(-c4cc5nc(-c6ccccc6)oc5c5ccccc45)cc3)c3ccc4c(c3)oc3ccccc34)ccc21 ; CC1(C)c2ccccc2-c2cc(N(c3ccc(-c4cc5nc(-c6ccccc6)oc5c5ccccc45)cc3)c3ccc4oc5ccccc5c4c3)ccc21 ; CC1(C)c2ccccc2-c2ccc(N(c3ccc(-c4cc5nc(-c6ccccc6)oc5c5ccccc45)cc3)c3ccc4c(c3)sc3ccccc34)cc21]

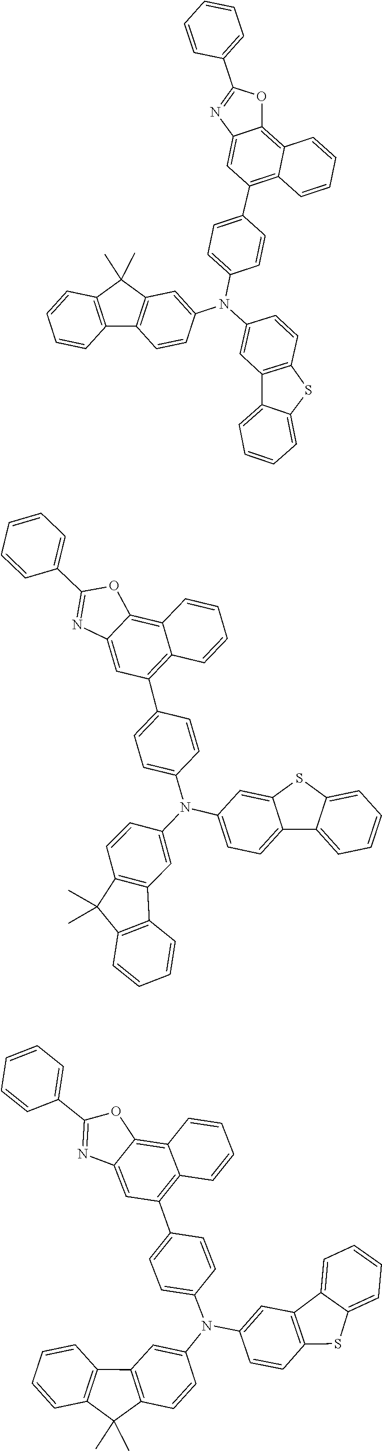 [SMILES: CC1(C)c2ccccc2-c2ccc(N(c3ccc(-c4cc5nc(-c6ccccc6)oc5c5ccccc45)cc3)c3ccc4sc5ccccc5c4c3)cc21 ; CC1(C)c2ccccc2-c2cc(N(c3ccc(-c4cc5nc(-c6ccccc6)oc5c5ccccc45)cc3)c3ccc4c(c3)sc3ccccc34)ccc21 ; CC1(C)c2ccccc2-c2cc(N(c3ccc(-c4cc5nc(-c6ccccc6)oc5c5ccccc45)cc3)c3ccc4sc5ccccc5c4c3)ccc21]

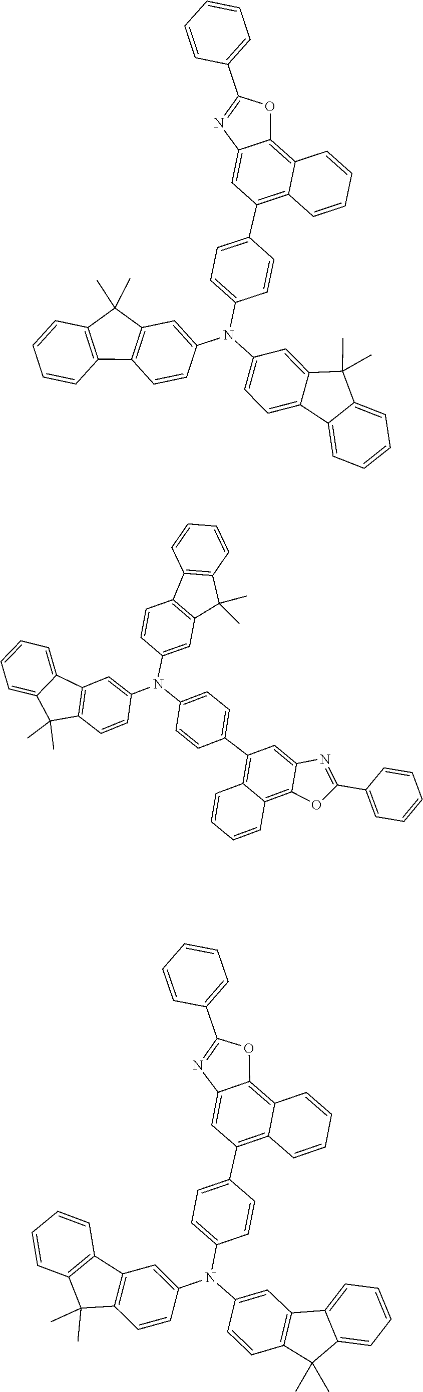 [SMILES: CC1(C)c2ccccc2-c2ccc(N(c3ccc(-c4cc5nc(-c6ccccc6)oc5c5ccccc45)cc3)c3ccc4c(c3)C(C)(C)c3ccccc3-4)cc21 ; CC1(C)c2ccccc2-c2cc(N(c3ccc(-c4cc5nc(-c6ccccc6)oc5c5ccccc45)cc3)c3ccc4c(c3)C(C)(C)c3ccccc3-4)ccc21 ; CC1(C)c2ccccc2-c2cc(N(c3ccc(-c4cc5nc(-c6ccccc6)oc5c5ccccc45)cc3)c3ccc4c(c3)-c3ccccc3C4(C)C)ccc21]

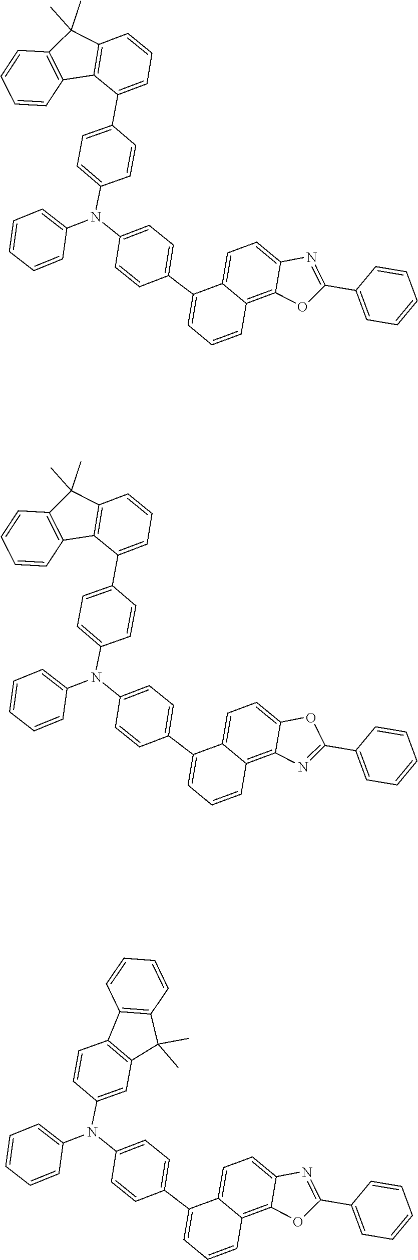 [SMILES: CC1(C)c2ccccc2-c2c(-c3ccc(N(c4ccccc4)c4ccc(-c5cccc6c5ccc5nc(-c7ccccc7)oc56)cc4)cc3)cccc21 ; CC1(C)c2ccccc2-c2c(-c3ccc(N(c4ccccc4)c4ccc(-c5cccc6c5ccc5oc(-c7ccccc7)nc56)cc4)cc3)cccc21 ; CC1(C)c2ccccc2-c2ccc(N(c3ccccc3)c3ccc(-c4cccc5c4ccc4nc(-c6ccccc6)oc45)cc3)cc21]

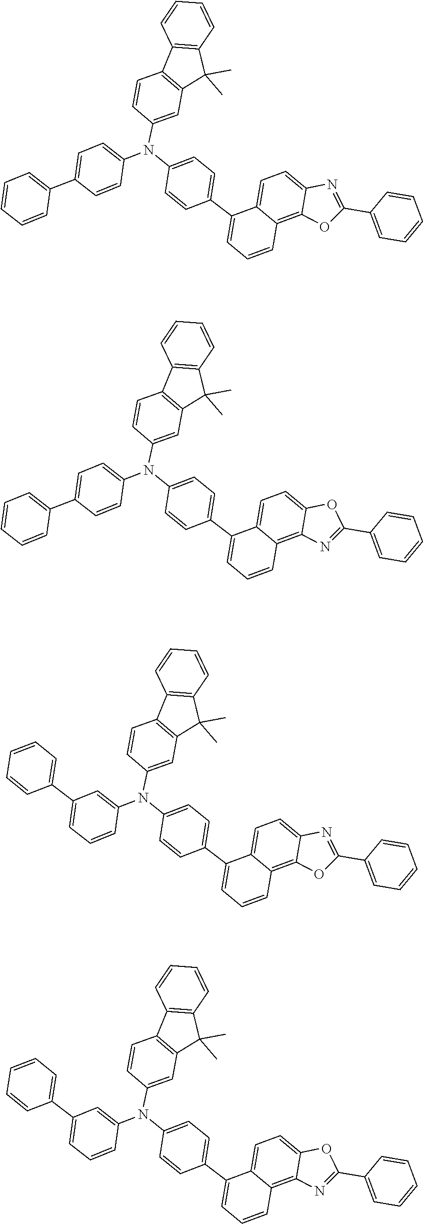 [SMILES: CC1(C)c2ccccc2-c2ccc(N(c3ccc(-c4ccccc4)cc3)c3ccc(-c4cccc5c4ccc4nc(-c6ccccc6)oc45)cc3)cc21 ; CC1(C)c2ccccc2-c2ccc(N(c3ccc(-c4ccccc4)cc3)c3ccc(-c4cccc5c4ccc4oc(-c6ccccc6)nc45)cc3)cc21 ; CC1(C)c2ccccc2-c2ccc(N(c3ccc(-c4cccc5c4ccc4nc(-c6ccccc6)oc45)cc3)c3cccc(-c4ccccc4)c3)cc21 ; CC1(C)c2ccccc2-c2ccc(N(c3ccc(-c4cccc5c4ccc4oc(-c6ccccc6)nc45)cc3)c3cccc(-c4ccccc4)c3)cc21]

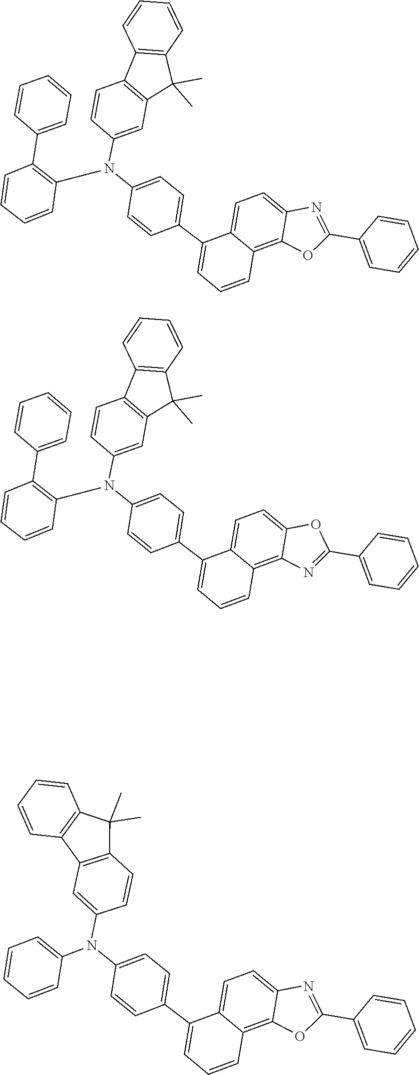 [SMILES: CC1(C)c2ccccc2-c2ccc(N(c3ccc(-c4cccc5c4ccc4nc(-c6ccccc6)oc45)cc3)c3ccccc3-c3ccccc3)cc21 ; CC1(C)c2ccccc2-c2ccc(N(c3ccc(-c4cccc5c4ccc4oc(-c6ccccc6)nc45)cc3)c3ccccc3-c3ccccc3)cc21 ; CC1(C)c2ccccc2-c2cc(N(c3ccccc3)c3ccc(-c4cccc5c4ccc4nc(-c6ccccc6)oc45)cc3)ccc21]

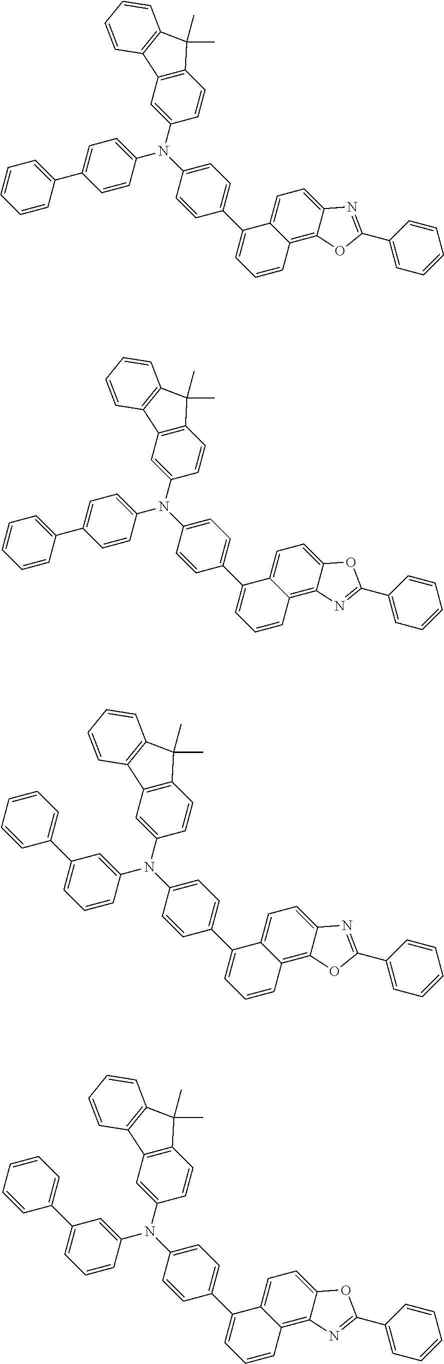 [SMILES: CC1(C)c2ccccc2-c2cc(N(c3ccc(-c4ccccc4)cc3)c3ccc(-c4cccc5c4ccc4nc(-c6ccccc6)oc45)cc3)ccc21 ; CC1(C)c2ccccc2-c2cc(N(c3ccc(-c4ccccc4)cc3)c3ccc(-c4cccc5c4ccc4oc(-c6ccccc6)nc45)cc3)ccc21 ; CC1(C)c2ccccc2-c2cc(N(c3ccc(-c4cccc5c4ccc4nc(-c6ccccc6)oc45)cc3)c3cccc(-c4ccccc4)c3)ccc21 ; CC1(C)c2ccccc2-c2cc(N(c3ccc(-c4cccc5c4ccc4oc(-c6ccccc6)nc45)cc3)c3cccc(-c4ccccc4)c3)ccc21]

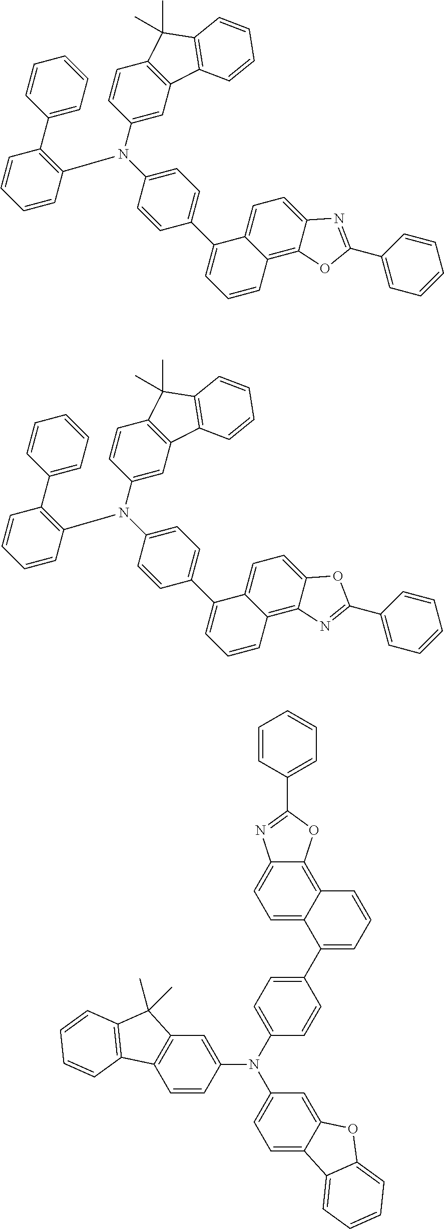 [SMILES: CC1(C)c2ccccc2-c2cc(N(c3ccc(-c4cccc5c4ccc4nc(-c6ccccc6)oc45)cc3)c3ccccc3-c3ccccc3)ccc21 ; CC1(C)c2ccccc2-c2cc(N(c3ccc(-c4cccc5c4ccc4oc(-c6ccccc6)nc45)cc3)c3ccccc3-c3ccccc3)ccc21 ; CC1(C)c2ccccc2-c2ccc(N(c3ccc(-c4cccc5c4ccc4nc(-c6ccccc6)oc45)cc3)c3ccc4c(c3)oc3ccccc34)cc21]

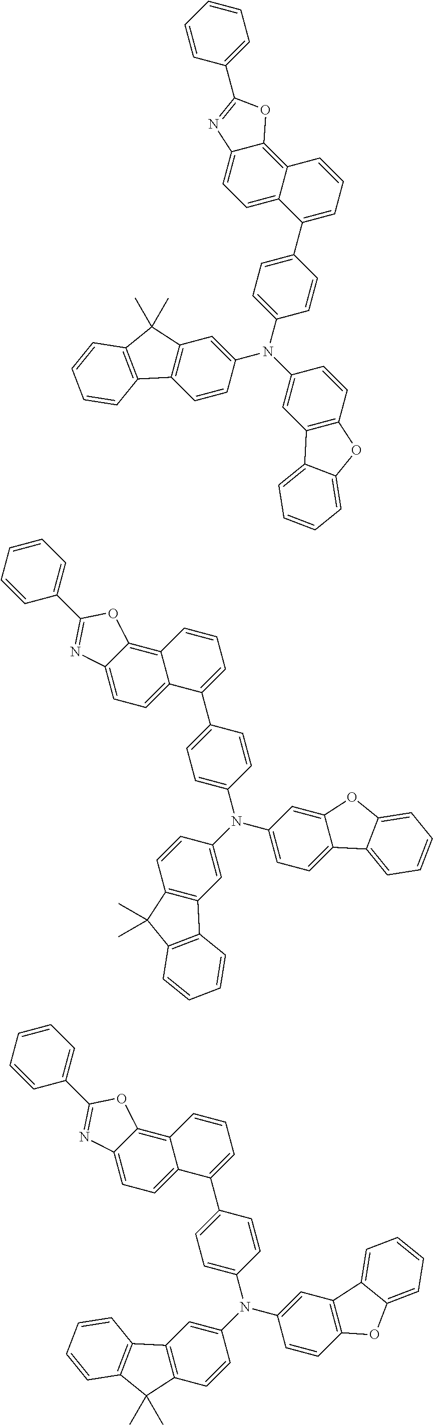 [SMILES: CC1(C)c2ccccc2-c2ccc(N(c3ccc(-c4cccc5c4ccc4nc(-c6ccccc6)oc45)cc3)c3ccc4oc5ccccc5c4c3)cc21 ; CC1(C)c2ccccc2-c2cc(N(c3ccc(-c4cccc5c4ccc4nc(-c6ccccc6)oc45)cc3)c3ccc4c(c3)oc3ccccc34)ccc21 ; CC1(C)c2ccccc2-c2cc(N(c3ccc(-c4cccc5c4ccc4nc(-c6ccccc6)oc45)cc3)c3ccc4oc5ccccc5c4c3)ccc21]

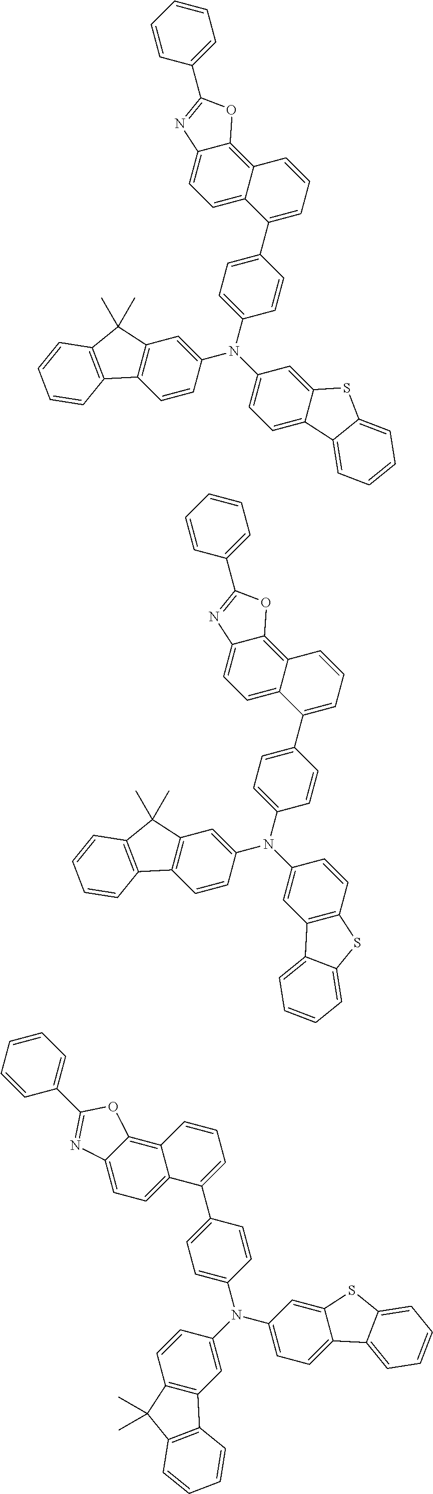 [SMILES: CC1(C)c2ccccc2-c2ccc(N(c3ccc(-c4cccc5c4ccc4nc(-c6ccccc6)oc45)cc3)c3ccc4c(c3)sc3ccccc34)cc21 ; CC1(C)c2ccccc2-c2ccc(N(c3ccc(-c4cccc5c4ccc4nc(-c6ccccc6)oc45)cc3)c3ccc4sc5ccccc5c4c3)cc21 ; CC1(C)c2ccccc2-c2cc(N(c3ccc(-c4cccc5c4ccc4nc(-c6ccccc6)oc45)cc3)c3ccc4c(c3)sc3ccccc34)ccc21]

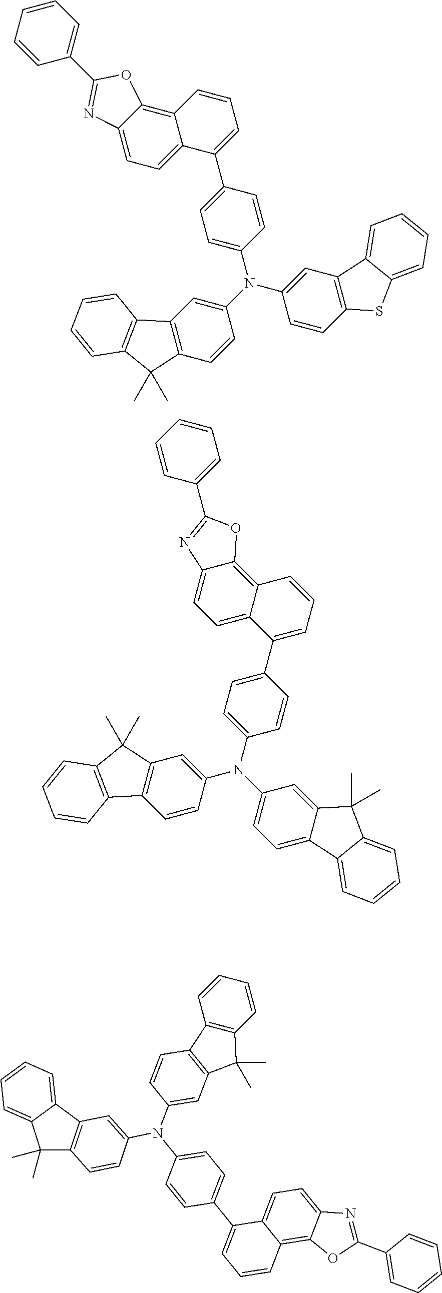

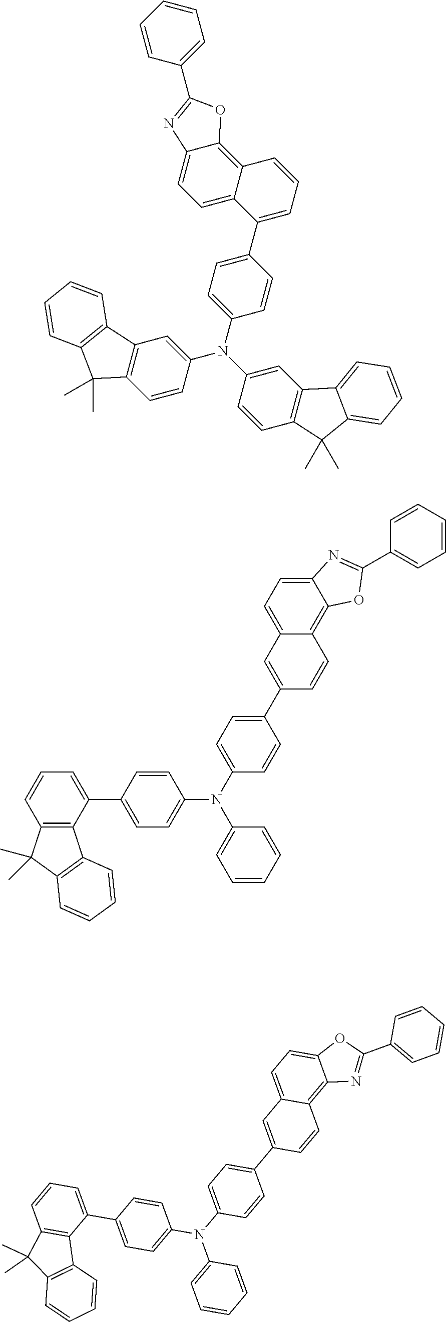 [SMILES: CC1(C)c2ccccc2-c2cc(N(c3ccc(-c4cccc5c4ccc4nc(-c6ccccc6)oc45)cc3)c3ccc4c(c3)-c3ccccc3C4(C)C)ccc21 ; CC1(C)c2ccccc2-c2c(-c3ccc(N(c4ccccc4)c4ccc(-c5ccc6c(ccc7nc(-c8ccccc8)oc76)c5)cc4)cc3)cccc21 ; CC1(C)c2ccccc2-c2c(-c3ccc(N(c4ccccc4)c4ccc(-c5ccc6c(ccc7oc(-c8ccccc8)nc76)c5)cc4)cc3)cccc21]

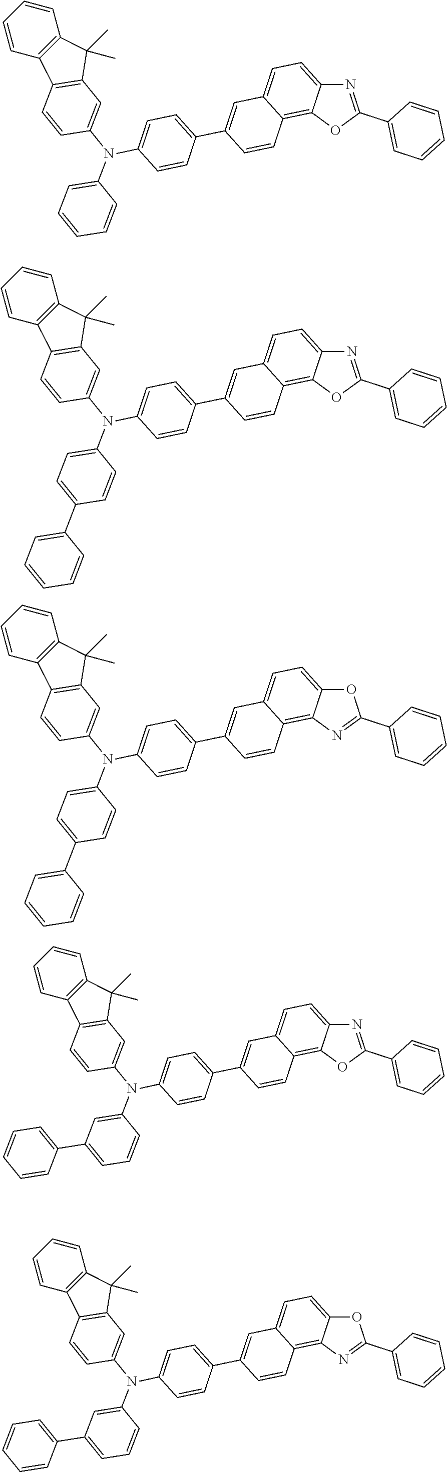

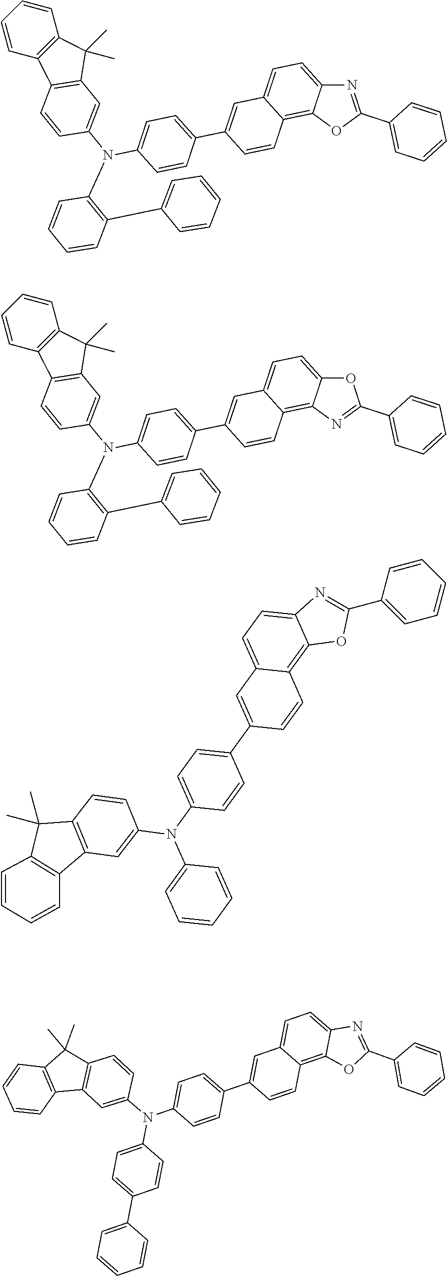 [SMILES: CC1(C)c2ccccc2-c2ccc(N(c3ccc(-c4ccc5c(ccc6nc(-c7ccccc7)oc65)c4)cc3)c3ccccc3-c3ccccc3)cc21 ; CC1(C)c2ccccc2-c2ccc(N(c3ccc(-c4ccc5c(ccc6oc(-c7ccccc7)nc65)c4)cc3)c3ccccc3-c3ccccc3)cc21 ; CC1(C)c2ccccc2-c2cc(N(c3ccccc3)c3ccc(-c4ccc5c(ccc6nc(-c7ccccc7)oc65)c4)cc3)ccc21 ; CC1(C)c2ccccc2-c2cc(N(c3ccc(-c4ccccc4)cc3)c3ccc(-c4ccc5c(ccc6nc(-c7ccccc7)oc65)c4)cc3)ccc21]

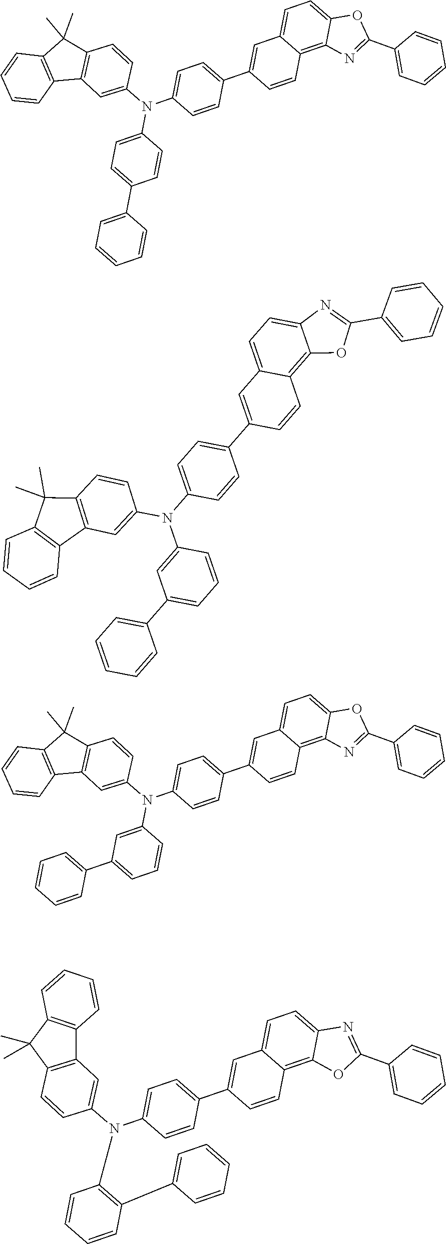 [SMILES: CC1(C)c2ccccc2-c2cc(N(c3ccc(-c4ccccc4)cc3)c3ccc(-c4ccc5c(ccc6oc(-c7ccccc7)nc65)c4)cc3)ccc21 ; CC1(C)c2ccccc2-c2cc(N(c3ccc(-c4ccc5c(ccc6nc(-c7ccccc7)oc65)c4)cc3)c3cccc(-c4ccccc4)c3)ccc21 ; CC1(C)c2ccccc2-c2cc(N(c3ccc(-c4ccc5c(ccc6oc(-c7ccccc7)nc65)c4)cc3)c3cccc(-c4ccccc4)c3)ccc21 ; CC1(C)c2ccccc2-c2cc(N(c3ccc(-c4ccc5c(ccc6nc(-c7ccccc7)oc65)c4)cc3)c3ccccc3-c3ccccc3)ccc21]

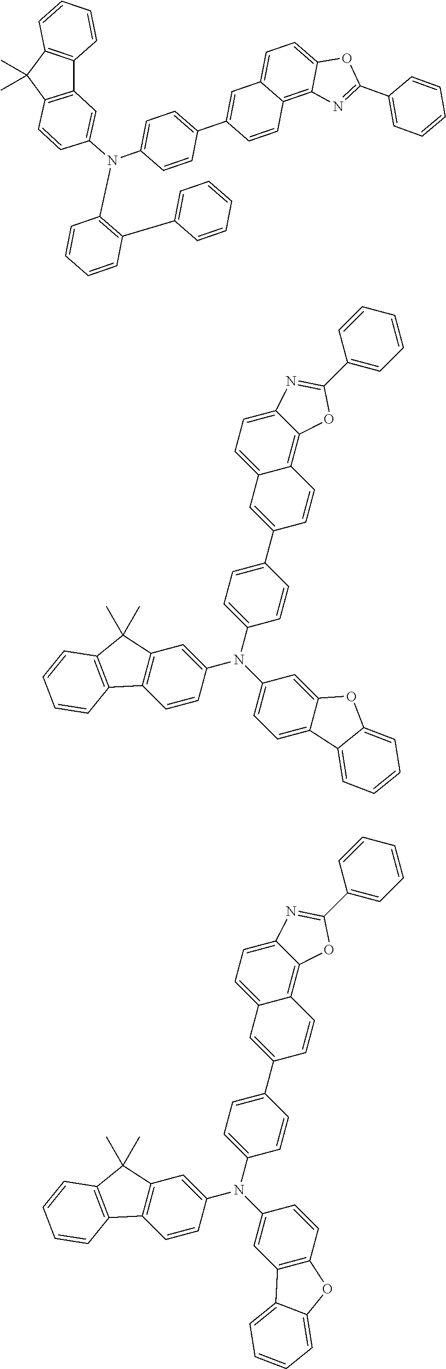

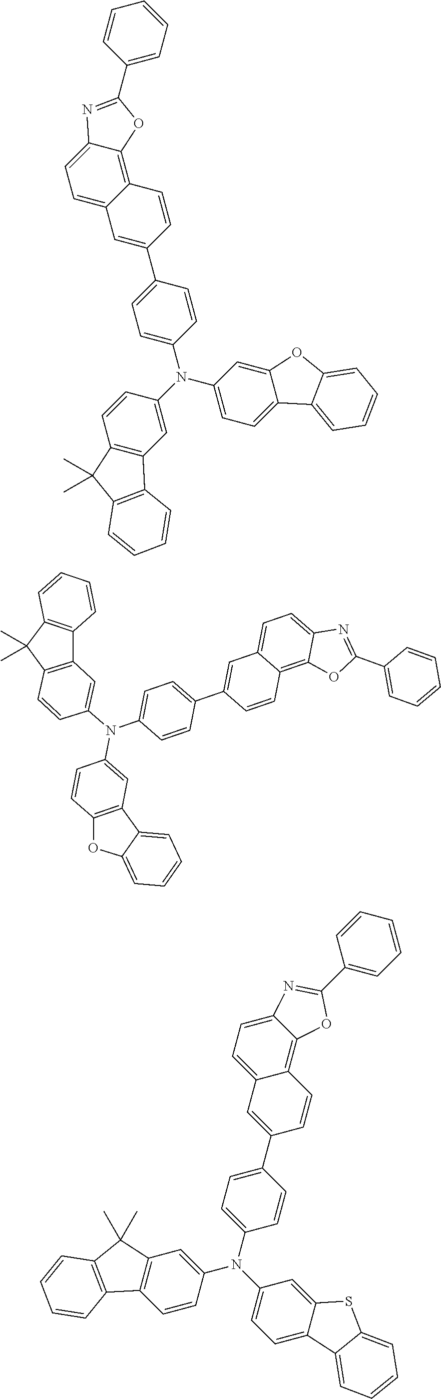 [SMILES: CC1(C)c2ccccc2-c2cc(N(c3ccc(-c4ccc5c(ccc6nc(-c7ccccc7)oc65)c4)cc3)c3ccc4c(c3)oc3ccccc34)ccc21 ; CC1(C)c2ccccc2-c2cc(N(c3ccc(-c4ccc5c(ccc6nc(-c7ccccc7)oc65)c4)cc3)c3ccc4oc5ccccc5c4c3)ccc21 ; CC1(C)c2ccccc2-c2ccc(N(c3ccc(-c4ccc5c(ccc6nc(-c7ccccc7)oc65)c4)cc3)c3ccc4c(c3)sc3ccccc34)cc21]

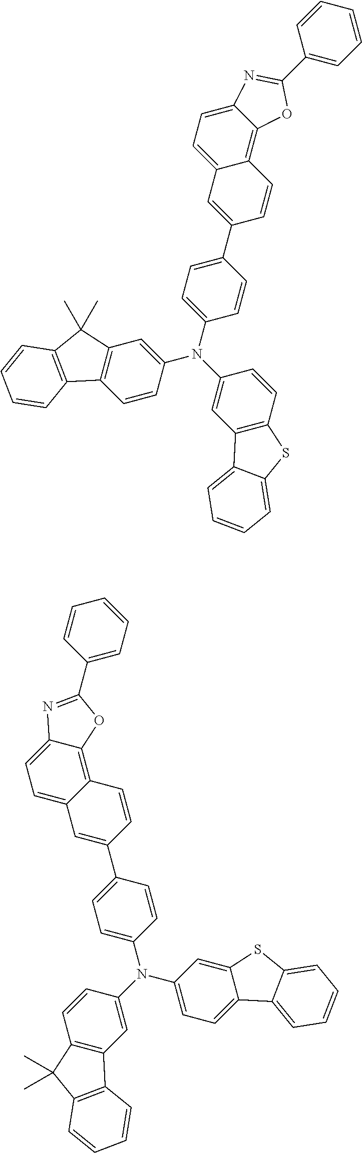 [SMILES: CC1(C)c2ccccc2-c2ccc(N(c3ccc(-c4ccc5c(ccc6nc(-c7ccccc7)oc65)c4)cc3)c3ccc4sc5ccccc5c4c3)cc21 ; CC1(C)c2ccccc2-c2cc(N(c3ccc(-c4ccc5c(ccc6nc(-c7ccccc7)oc65)c4)cc3)c3ccc4c(c3)sc3ccccc34)ccc21]

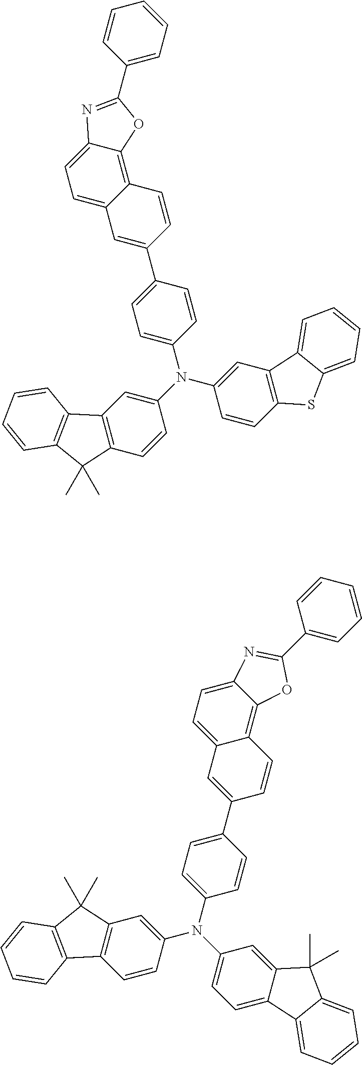 [SMILES: CC1(C)c2ccccc2-c2cc(N(c3ccc(-c4ccc5c(ccc6nc(-c7ccccc7)oc65)c4)cc3)c3ccc4sc5ccccc5c4c3)ccc21 ; CC1(C)c2ccccc2-c2ccc(N(c3ccc(-c4ccc5c(ccc6nc(-c7ccccc7)oc65)c4)cc3)c3ccc4c(c3)C(C)(C)c3ccccc3-4)cc21]

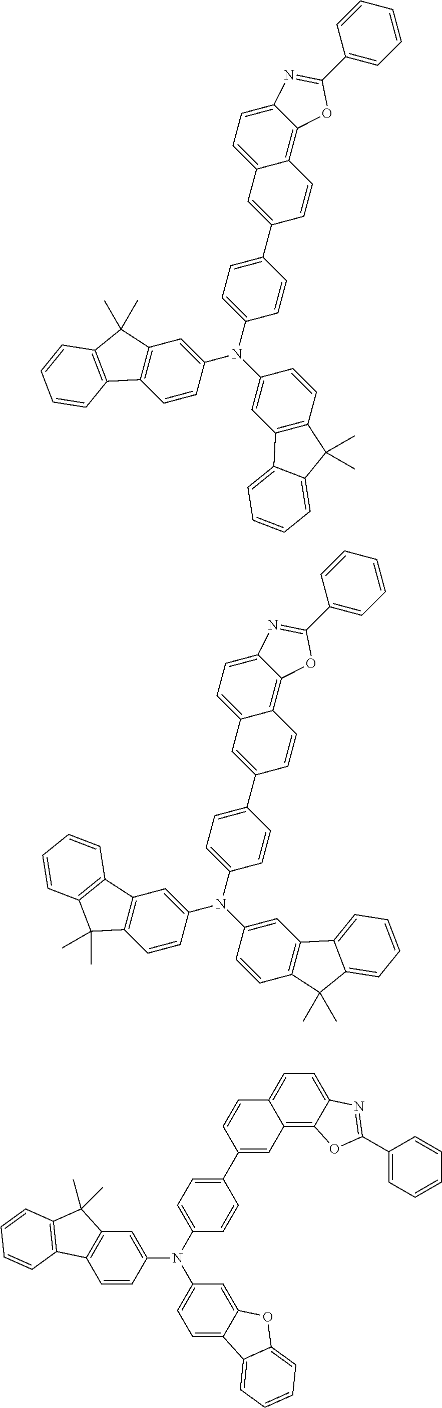 [SMILES: CC1(C)c2ccccc2-c2cc(N(c3ccc(-c4ccc5c(ccc6nc(-c7ccccc7)oc65)c4)cc3)c3ccc4c(c3)C(C)(C)c3ccccc3-4)ccc21 ; CC1(C)c2ccccc2-c2cc(N(c3ccc(-c4ccc5c(ccc6nc(-c7ccccc7)oc65)c4)cc3)c3ccc4c(c3)-c3ccccc3C4(C)C)ccc21 ; CC1(C)c2ccccc2-c2ccc(N(c3ccc(-c4ccc5ccc6nc(-c7ccccc7)oc6c5c4)cc3)c3ccc4c(c3)oc3ccccc34)cc21]

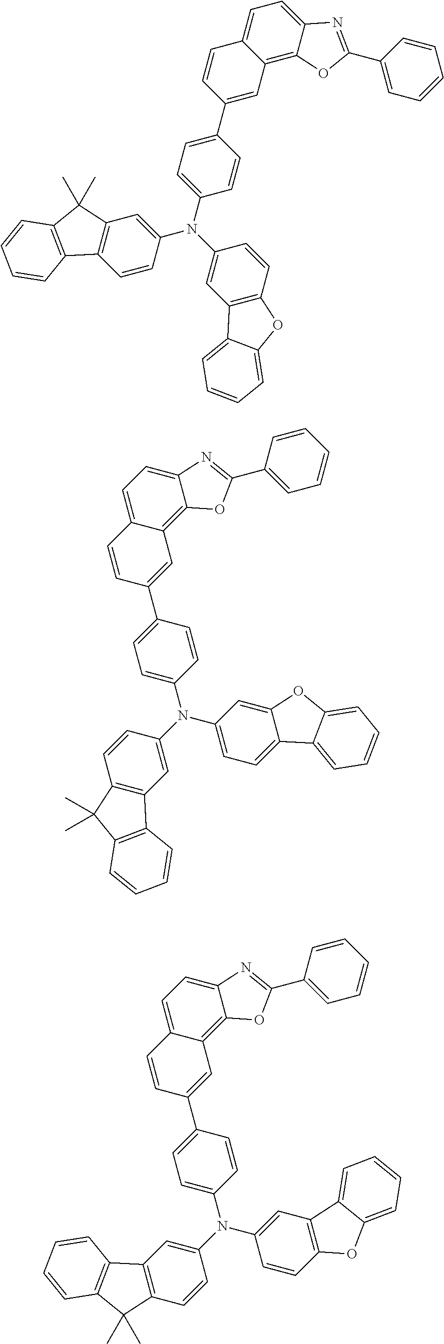 [SMILES: CC1(C)c2ccccc2-c2ccc(N(c3ccc(-c4ccc5ccc6nc(-c7ccccc7)oc6c5c4)cc3)c3ccc4oc5ccccc5c4c3)cc21 ; CC1(C)c2ccccc2-c2cc(N(c3ccc(-c4ccc5ccc6nc(-c7ccccc7)oc6c5c4)cc3)c3ccc4c(c3)oc3ccccc34)ccc21 ; CC1(C)c2ccccc2-c2cc(N(c3ccc(-c4ccc5ccc6nc(-c7ccccc7)oc6c5c4)cc3)c3ccc4oc5ccccc5c4c3)ccc21]

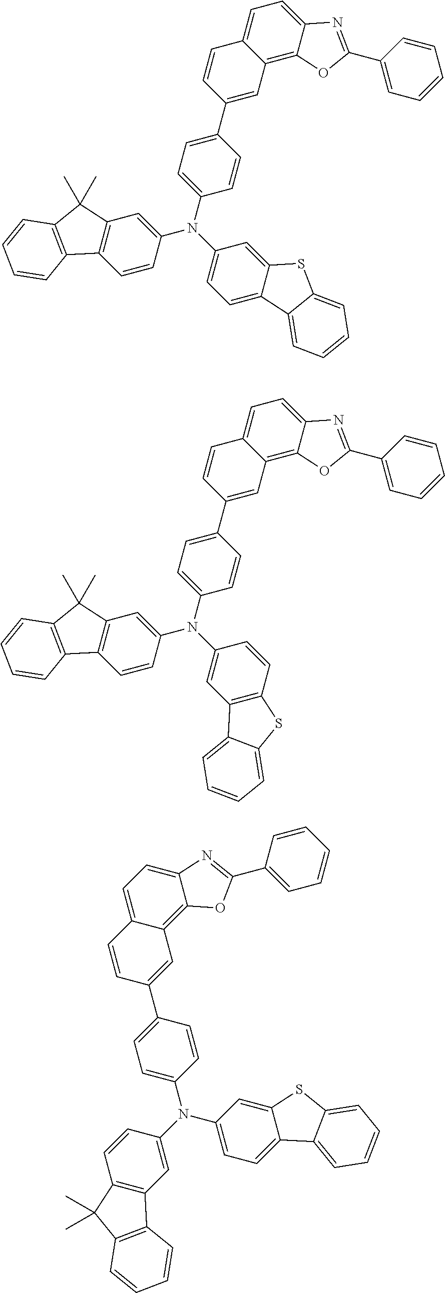 [SMILES: CC1(C)c2ccccc2-c2ccc(N(c3ccc(-c4ccc5ccc6nc(-c7ccccc7)oc6c5c4)cc3)c3ccc4c(c3)sc3ccccc34)cc21 ; CC1(C)c2ccccc2-c2ccc(N(c3ccc(-c4ccc5ccc6nc(-c7ccccc7)oc6c5c4)cc3)c3ccc4sc5ccccc5c4c3)cc21 ; CC1(C)c2ccccc2-c2cc(N(c3ccc(-c4ccc5ccc6nc(-c7ccccc7)oc6c5c4)cc3)c3ccc4c(c3)sc3ccccc34)ccc21]

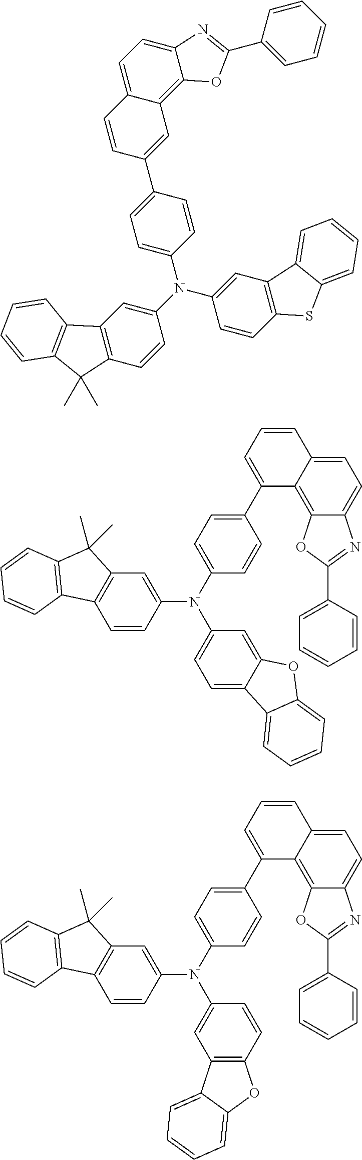

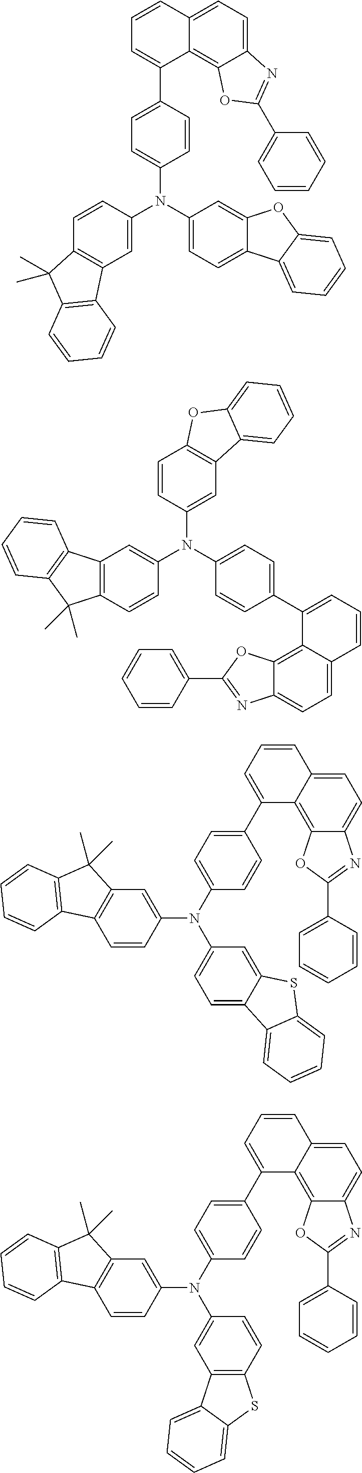

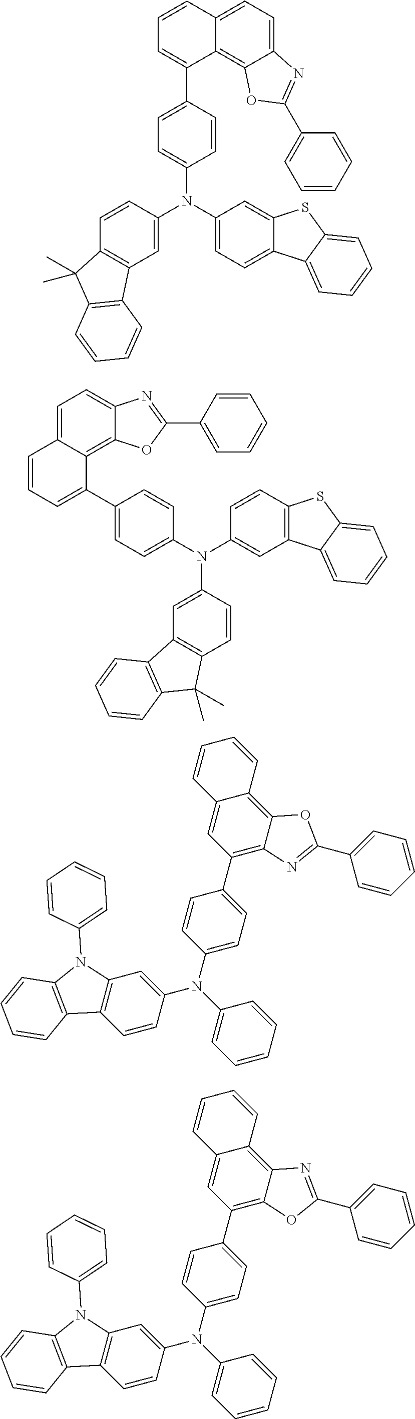

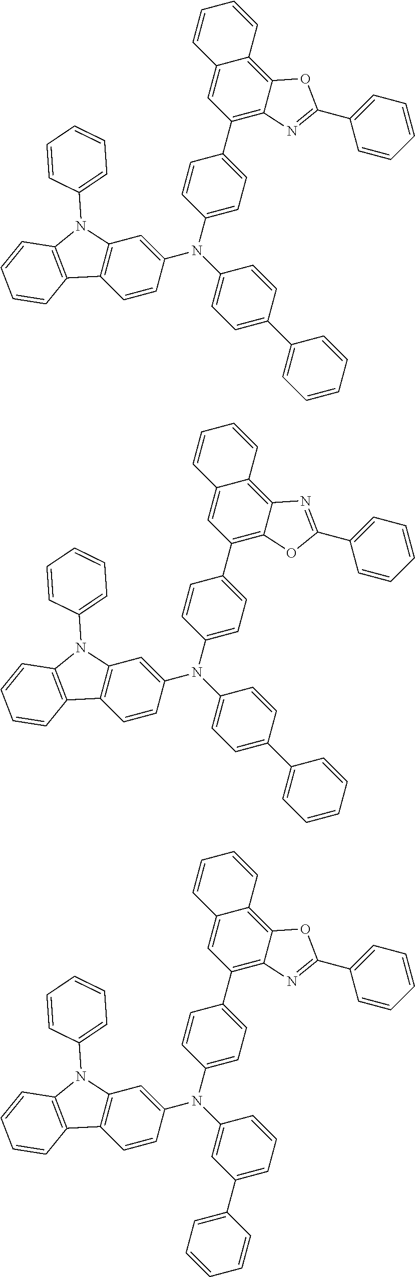 [SMILES: c1ccc(-c2ccc(N(c3ccc(-c4cc5ccccc5c5oc(-c6ccccc6)nc45)cc3)c3ccc4c5ccccc5n(-c5ccccc5)c4c3)cc2)cc1 ; c1ccc(-c2ccc(N(c3ccc(-c4cc5ccccc5c5nc(-c6ccccc6)oc45)cc3)c3ccc4c5ccccc5n(-c5ccccc5)c4c3)cc2)cc1 ; c1ccc(-c2cccc(N(c3ccc(-c4cc5ccccc5c5oc(-c6ccccc6)nc45)cc3)c3ccc4c5ccccc5n(-c5ccccc5)c4c3)c2)cc1]

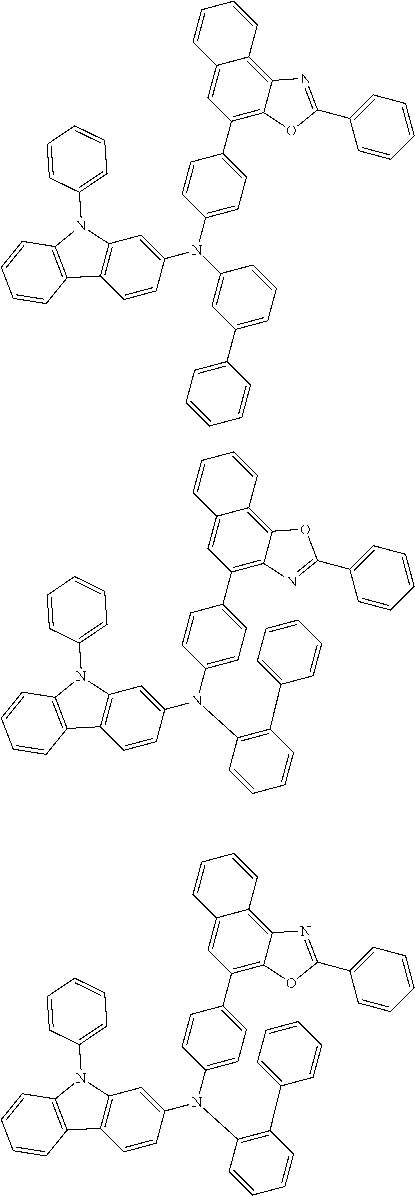

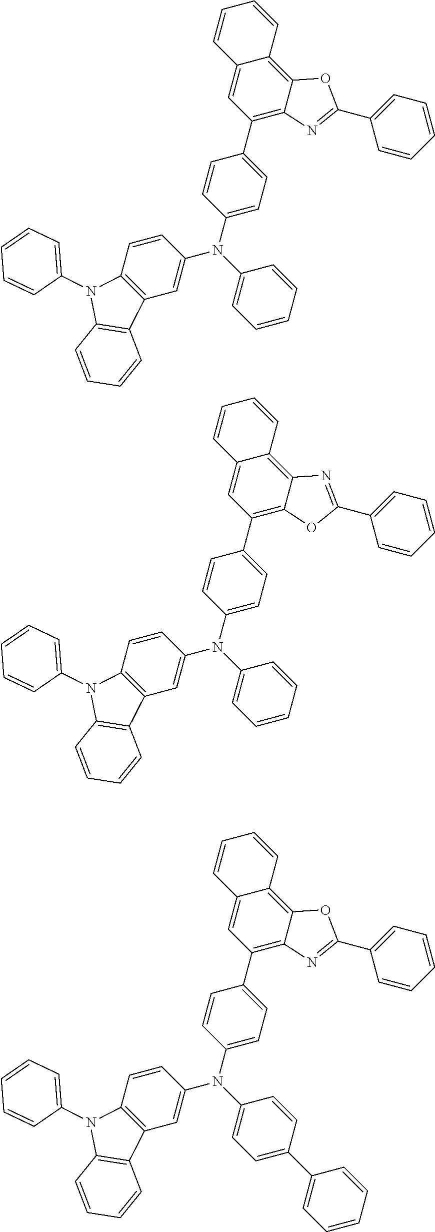 [SMILES: c1ccc(-c2nc3c(-c4ccc(N(c5ccccc5)c5ccc6c(c5)c5ccccc5n6-c5ccccc5)cc4)cc4ccccc4c3o2)cc1 ; c1ccc(-c2nc3c(o2)c(-c2ccc(N(c4ccccc4)c4ccc5c(c4)c4ccccc4n5-c4ccccc4)cc2)cc2ccccc23)cc1 ; c1ccc(-c2ccc(N(c3ccc(-c4cc5ccccc5c5oc(-c6ccccc6)nc45)cc3)c3ccc4c(c3)c3ccccc3n4-c3ccccc3)cc2)cc1]

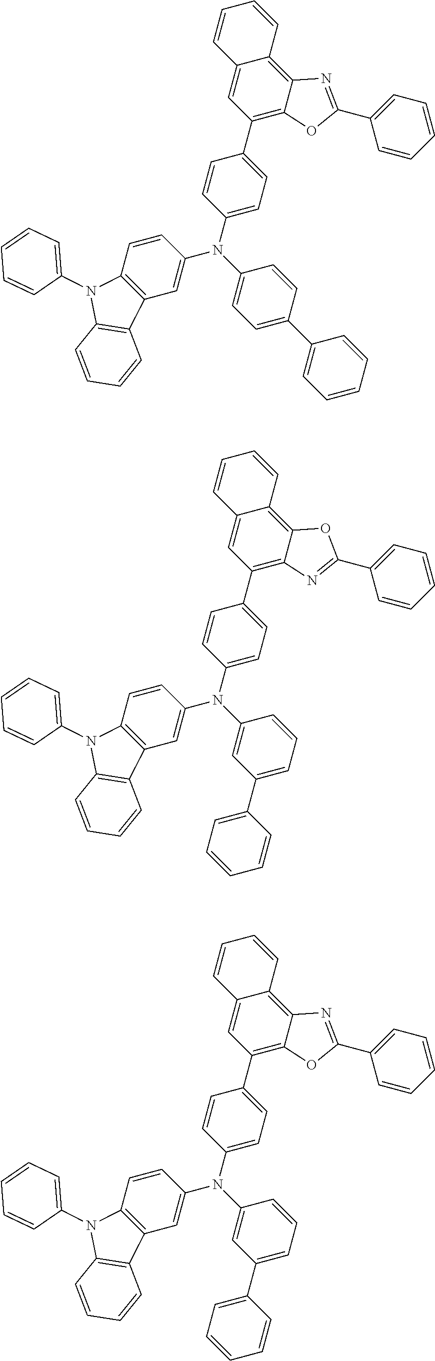 [SMILES: c1ccc(-c2ccc(N(c3ccc(-c4cc5ccccc5c5nc(-c6ccccc6)oc45)cc3)c3ccc4c(c3)c3ccccc3n4-c3ccccc3)cc2)cc1 ; c1ccc(-c2cccc(N(c3ccc(-c4cc5ccccc5c5oc(-c6ccccc6)nc45)cc3)c3ccc4c(c3)c3ccccc3n4-c3ccccc3)c2)cc1 ; c1ccc(-c2cccc(N(c3ccc(-c4cc5ccccc5c5nc(-c6ccccc6)oc45)cc3)c3ccc4c(c3)c3ccccc3n4-c3ccccc3)c2)cc1]

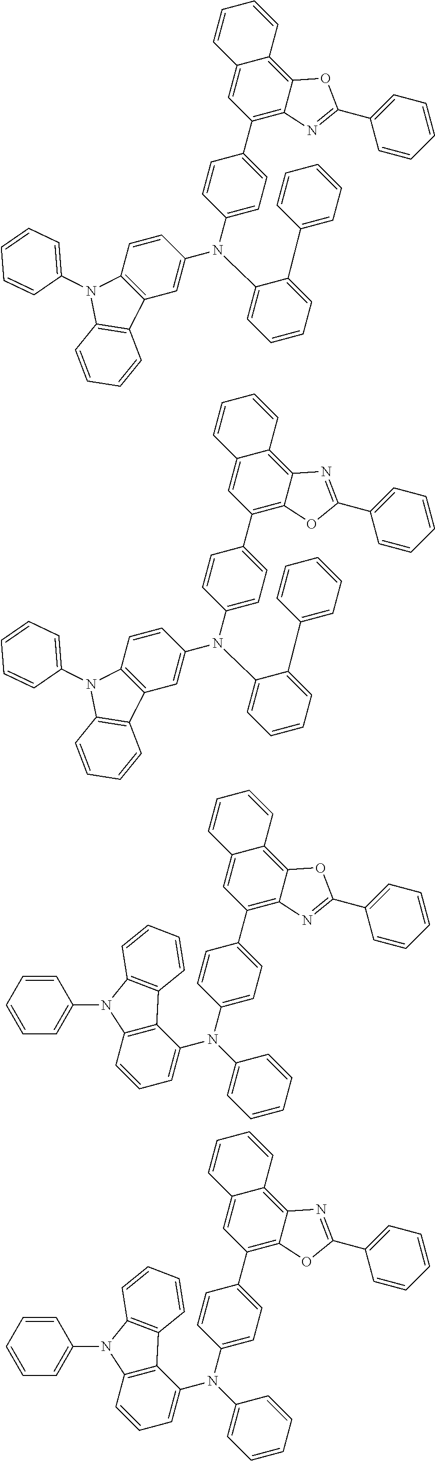

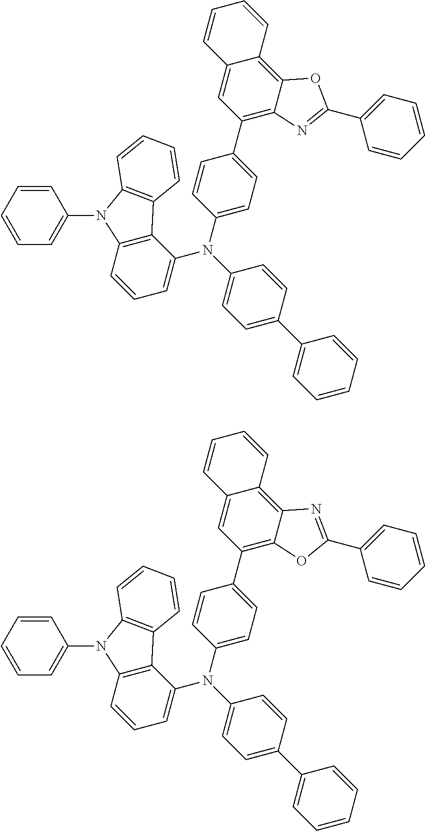 [SMILES: c1ccc(-c2ccc(N(c3ccc(-c4cc5ccccc5c5oc(-c6ccccc6)nc45)cc3)c3cccc4c3c3ccccc3n4-c3ccccc3)cc2)cc1 ; c1ccc(-c2ccc(N(c3ccc(-c4cc5ccccc5c5nc(-c6ccccc6)oc45)cc3)c3cccc4c3c3ccccc3n4-c3ccccc3)cc2)cc1]

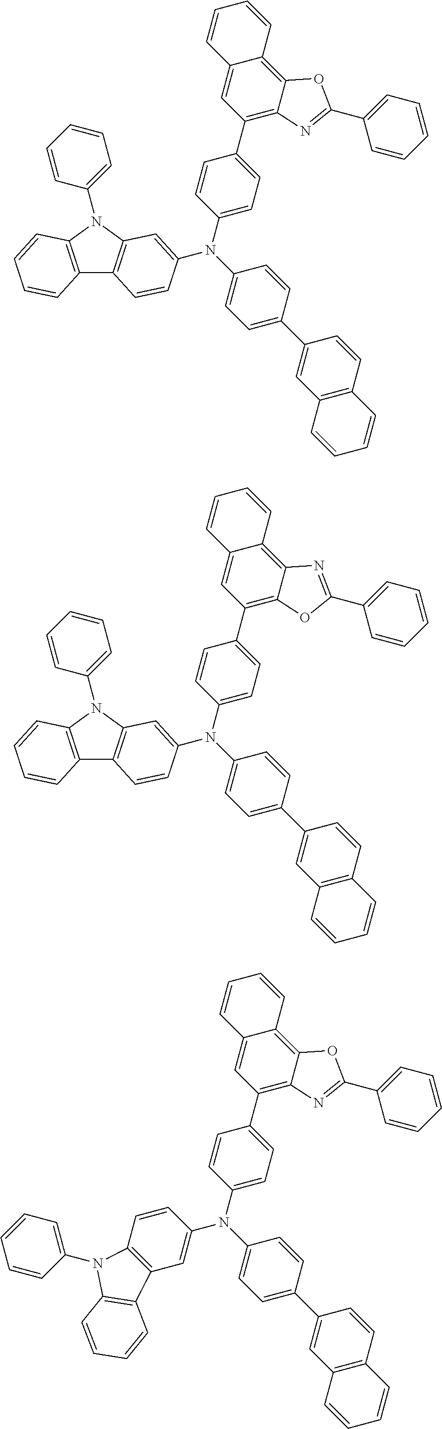 [SMILES: c1ccc(-c2nc3c(-c4ccc(N(c5ccc(-c6ccc7ccccc7c6)cc5)c5ccc6c7ccccc7n(-c7ccccc7)c6c5)cc4)cc4ccccc4c3o2)cc1 ; c1ccc(-c2nc3c(o2)c(-c2ccc(N(c4ccc(-c5ccc6ccccc6c5)cc4)c4ccc5c6ccccc6n(-c6ccccc6)c5c4)cc2)cc2ccccc23)cc1 ; c1ccc(-c2nc3c(-c4ccc(N(c5ccc(-c6ccc7ccccc7c6)cc5)c5ccc6c(c5)c5ccccc5n6-c5ccccc5)cc4)cc4ccccc4c3o2)cc1]

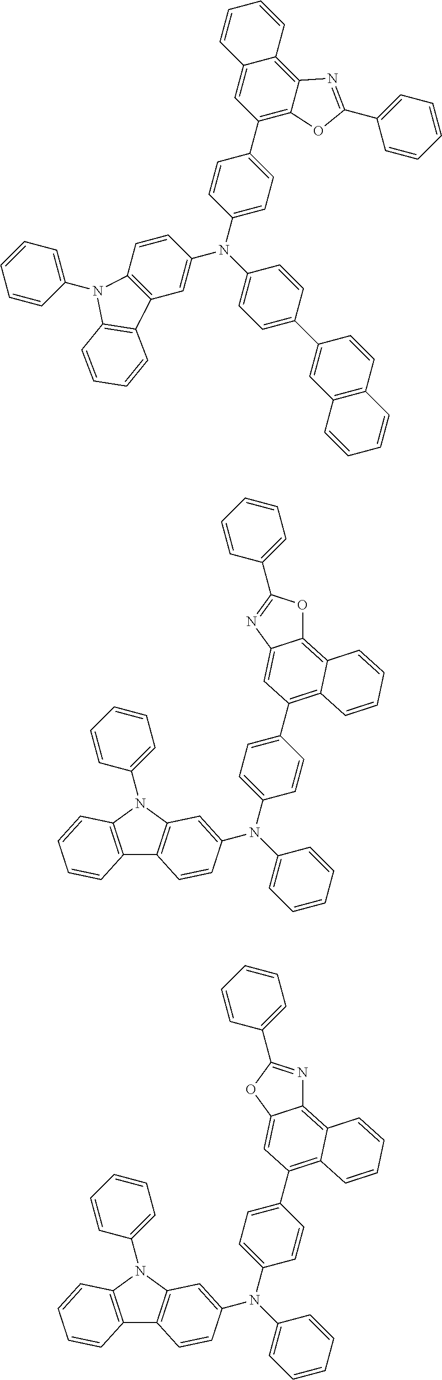 [SMILES: c1ccc(-c2nc3c(o2)c(-c2ccc(N(c4ccc(-c5ccc6ccccc6c5)cc4)c4ccc5c(c4)c4ccccc4n5-c4ccccc4)cc2)cc2ccccc23)cc1 ; c1ccc(-c2nc3cc(-c4ccc(N(c5ccccc5)c5ccc6c7ccccc7n(-c7ccccc7)c6c5)cc4)c4ccccc4c3o2)cc1 ; c1ccc(-c2nc3c(cc(-c4ccc(N(c5ccccc5)c5ccc6c7ccccc7n(-c7ccccc7)c6c5)cc4)c4ccccc43)o2)cc1]

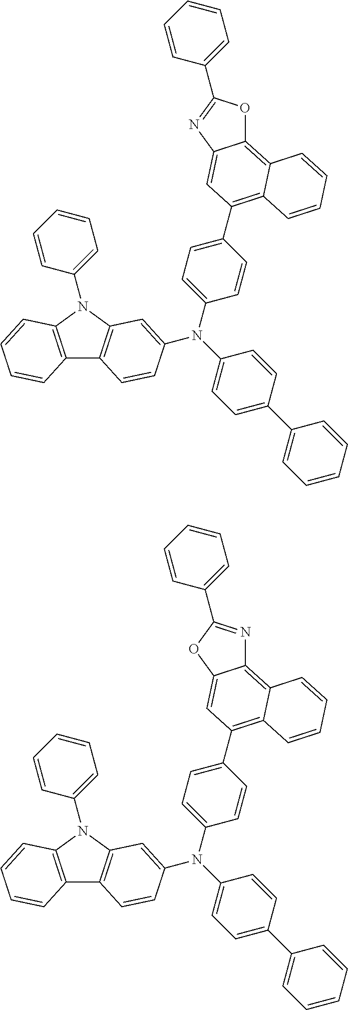 [SMILES: c1ccc(-c2ccc(N(c3ccc(-c4cc5nc(-c6ccccc6)oc5c5ccccc45)cc3)c3ccc4c5ccccc5n(-c5ccccc5)c4c3)cc2)cc1 ; c1ccc(-c2ccc(N(c3ccc(-c4cc5oc(-c6ccccc6)nc5c5ccccc45)cc3)c3ccc4c5ccccc5n(-c5ccccc5)c4c3)cc2)cc1]

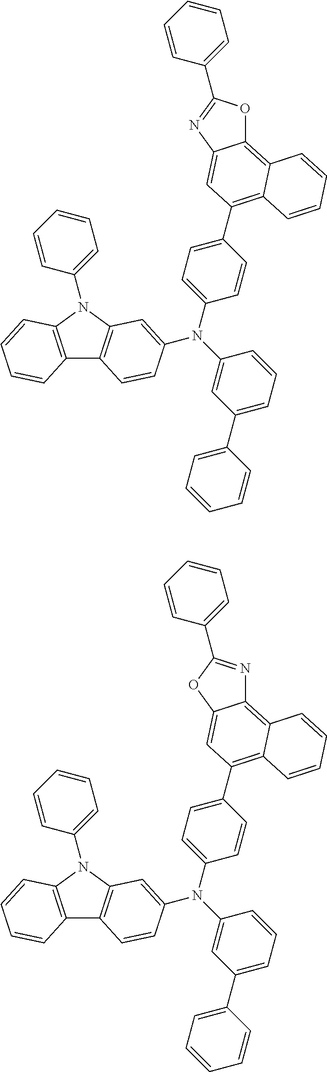 [SMILES: c1ccc(-c2cccc(N(c3ccc(-c4cc5nc(-c6ccccc6)oc5c5ccccc45)cc3)c3ccc4c5ccccc5n(-c5ccccc5)c4c3)c2)cc1 ; c1ccc(-c2cccc(N(c3ccc(-c4cc5oc(-c6ccccc6)nc5c5ccccc45)cc3)c3ccc4c5ccccc5n(-c5ccccc5)c4c3)c2)cc1]

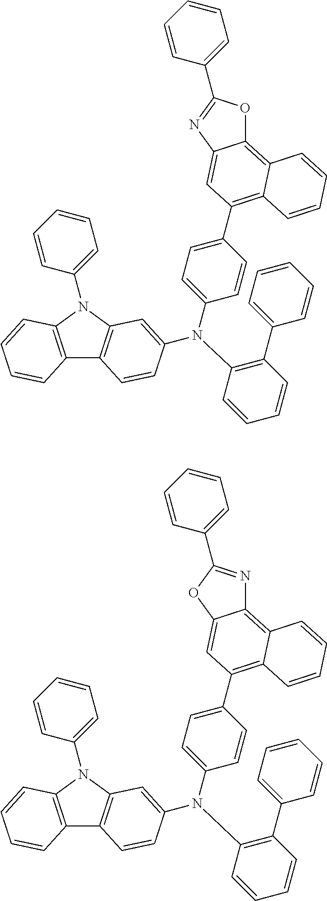 [SMILES: c1ccc(-c2nc3cc(-c4ccc(N(c5ccc6c7ccccc7n(-c7ccccc7)c6c5)c5ccccc5-c5ccccc5)cc4)c4ccccc4c3o2)cc1 ; c1ccc(-c2nc3c(cc(-c4ccc(N(c5ccc6c7ccccc7n(-c7ccccc7)c6c5)c5ccccc5-c5ccccc5)cc4)c4ccccc43)o2)cc1]

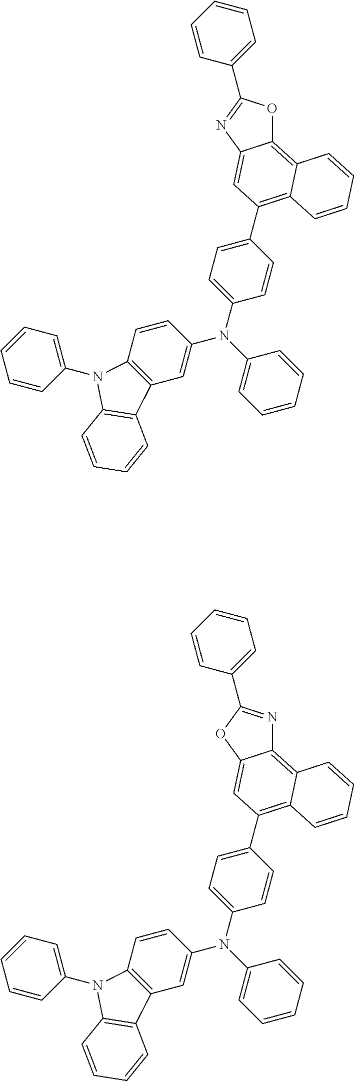 [SMILES: c1ccc(-c2nc3cc(-c4ccc(N(c5ccccc5)c5ccc6c(c5)c5ccccc5n6-c5ccccc5)cc4)c4ccccc4c3o2)cc1 ; c1ccc(-c2nc3c(cc(-c4ccc(N(c5ccccc5)c5ccc6c(c5)c5ccccc5n6-c5ccccc5)cc4)c4ccccc43)o2)cc1]

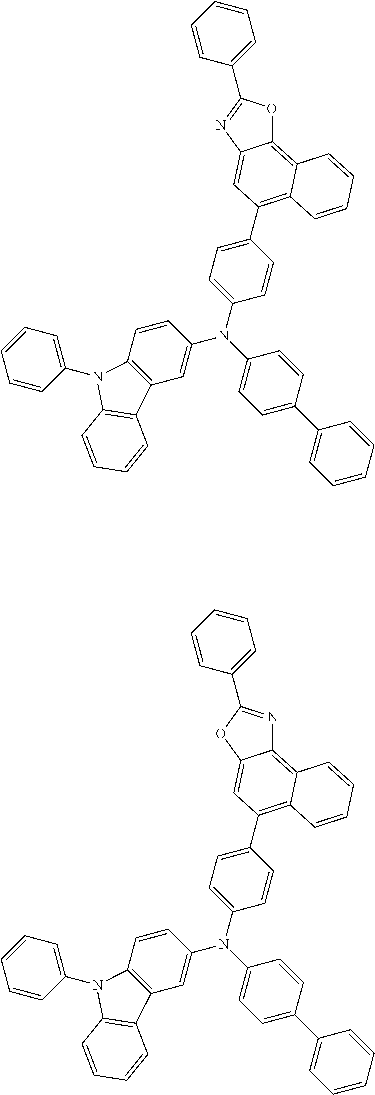 [SMILES: c1ccc(-c2ccc(N(c3ccc(-c4cc5nc(-c6ccccc6)oc5c5ccccc45)cc3)c3ccc4c(c3)c3ccccc3n4-c3ccccc3)cc2)cc1 ; c1ccc(-c2ccc(N(c3ccc(-c4cc5oc(-c6ccccc6)nc5c5ccccc45)cc3)c3ccc4c(c3)c3ccccc3n4-c3ccccc3)cc2)cc1]

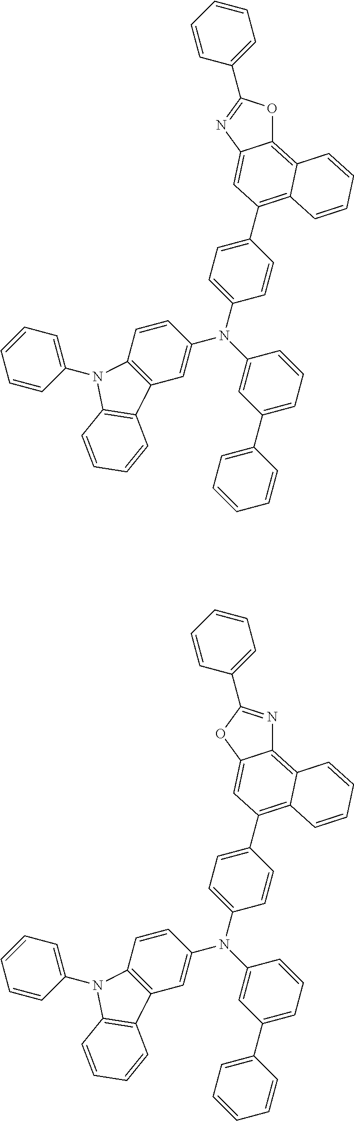 [SMILES: c1ccc(-c2cccc(N(c3ccc(-c4cc5nc(-c6ccccc6)oc5c5ccccc45)cc3)c3ccc4c(c3)c3ccccc3n4-c3ccccc3)c2)cc1 ; c1ccc(-c2cccc(N(c3ccc(-c4cc5oc(-c6ccccc6)nc5c5ccccc45)cc3)c3ccc4c(c3)c3ccccc3n4-c3ccccc3)c2)cc1]

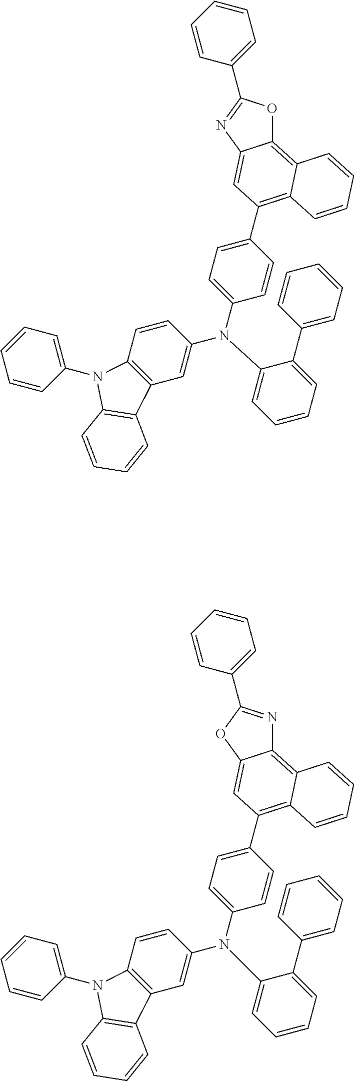 [SMILES: c1ccc(-c2nc3cc(-c4ccc(N(c5ccc6c(c5)c5ccccc5n6-c5ccccc5)c5ccccc5-c5ccccc5)cc4)c4ccccc4c3o2)cc1 ; c1ccc(-c2nc3c(cc(-c4ccc(N(c5ccc6c(c5)c5ccccc5n6-c5ccccc5)c5ccccc5-c5ccccc5)cc4)c4ccccc43)o2)cc1]

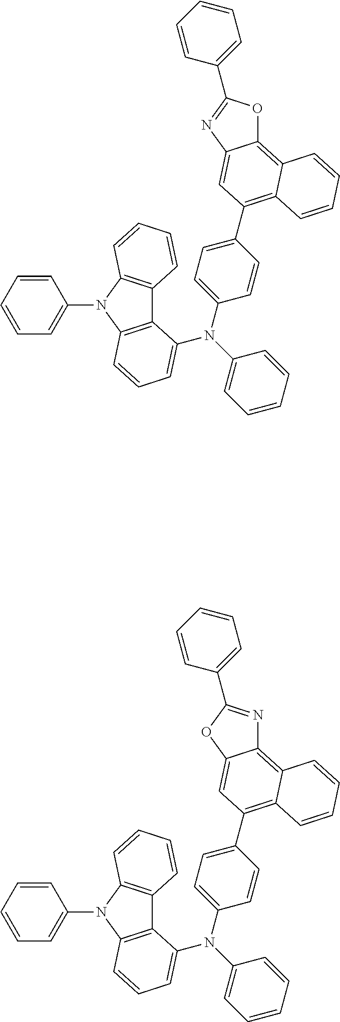 [SMILES: c1ccc(-c2nc3cc(-c4ccc(N(c5ccccc5)c5cccc6c5c5ccccc5n6-c5ccccc5)cc4)c4ccccc4c3o2)cc1 ; c1ccc(-c2nc3c(cc(-c4ccc(N(c5ccccc5)c5cccc6c5c5ccccc5n6-c5ccccc5)cc4)c4ccccc43)o2)cc1]

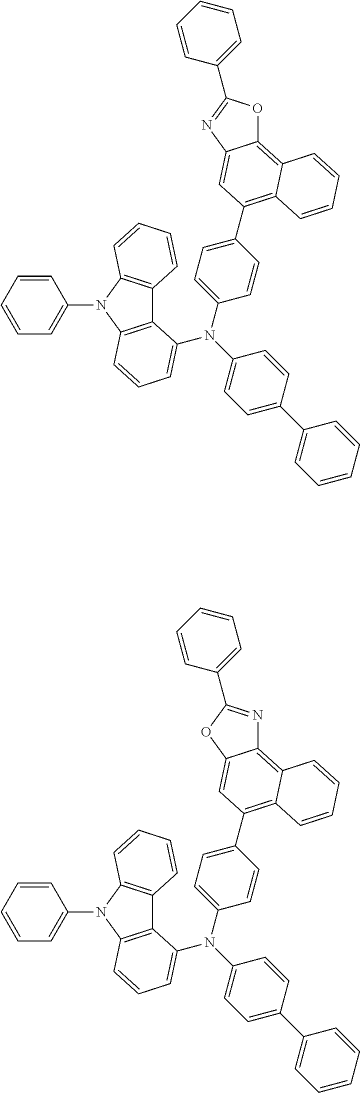 [SMILES: c1ccc(-c2ccc(N(c3ccc(-c4cc5nc(-c6ccccc6)oc5c5ccccc45)cc3)c3cccc4c3c3ccccc3n4-c3ccccc3)cc2)cc1 ; c1ccc(-c2ccc(N(c3ccc(-c4cc5oc(-c6ccccc6)nc5c5ccccc45)cc3)c3cccc4c3c3ccccc3n4-c3ccccc3)cc2)cc1]

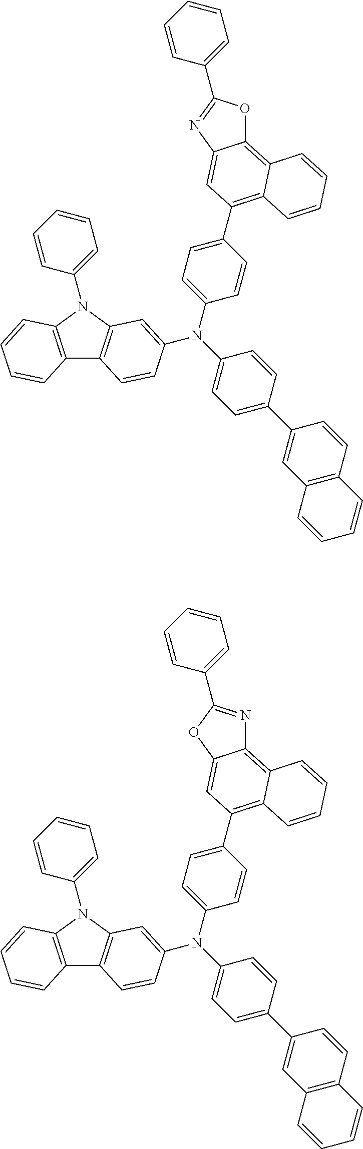 [SMILES: c1ccc(-c2nc3cc(-c4ccc(N(c5ccc(-c6ccc7ccccc7c6)cc5)c5ccc6c7ccccc7n(-c7ccccc7)c6c5)cc4)c4ccccc4c3o2)cc1 ; c1ccc(-c2nc3c(cc(-c4ccc(N(c5ccc(-c6ccc7ccccc7c6)cc5)c5ccc6c7ccccc7n(-c7ccccc7)c6c5)cc4)c4ccccc43)o2)cc1]

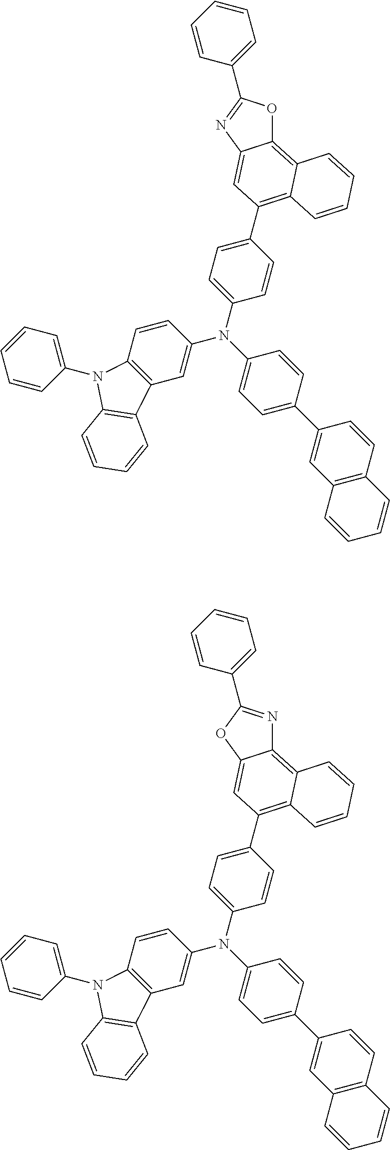 [SMILES: c1ccc(-c2nc3cc(-c4ccc(N(c5ccc(-c6ccc7ccccc7c6)cc5)c5ccc6c(c5)c5ccccc5n6-c5ccccc5)cc4)c4ccccc4c3o2)cc1 ; c1ccc(-c2nc3c(cc(-c4ccc(N(c5ccc(-c6ccc7ccccc7c6)cc5)c5ccc6c(c5)c5ccccc5n6-c5ccccc5)cc4)c4ccccc43)o2)cc1]

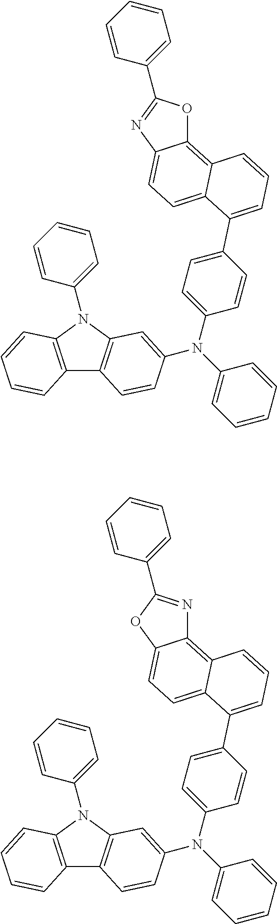 [SMILES: c1ccc(-c2nc3ccc4c(-c5ccc(N(c6ccccc6)c6ccc7c8ccccc8n(-c8ccccc8)c7c6)cc5)cccc4c3o2)cc1 ; c1ccc(-c2nc3c(ccc4c(-c5ccc(N(c6ccccc6)c6ccc7c8ccccc8n(-c8ccccc8)c7c6)cc5)cccc43)o2)cc1]

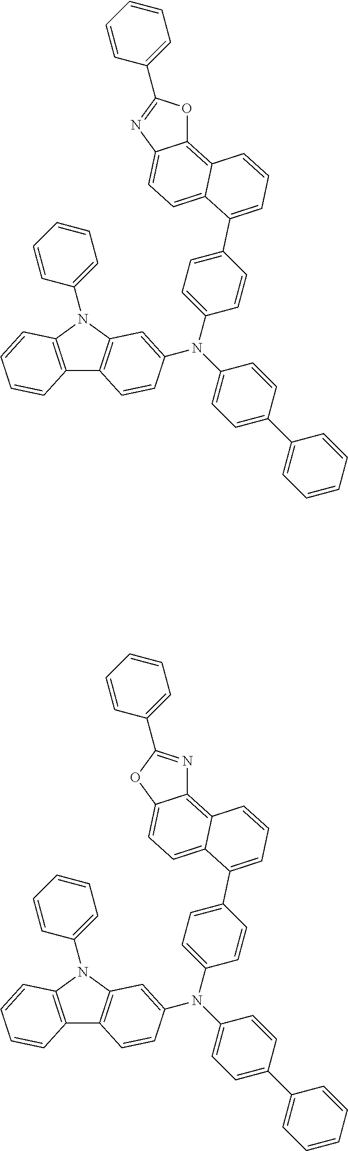 [SMILES: c1ccc(-c2ccc(N(c3ccc(-c4cccc5c4ccc4nc(-c6ccccc6)oc45)cc3)c3ccc4c5ccccc5n(-c5ccccc5)c4c3)cc2)cc1 ; c1ccc(-c2ccc(N(c3ccc(-c4cccc5c4ccc4oc(-c6ccccc6)nc45)cc3)c3ccc4c5ccccc5n(-c5ccccc5)c4c3)cc2)cc1]

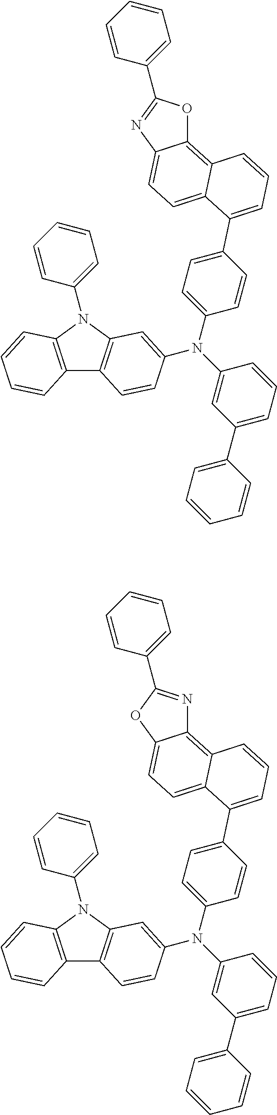 [SMILES: c1ccc(-c2cccc(N(c3ccc(-c4cccc5c4ccc4nc(-c6ccccc6)oc45)cc3)c3ccc4c5ccccc5n(-c5ccccc5)c4c3)c2)cc1 ; c1ccc(-c2cccc(N(c3ccc(-c4cccc5c4ccc4oc(-c6ccccc6)nc45)cc3)c3ccc4c5ccccc5n(-c5ccccc5)c4c3)c2)cc1]

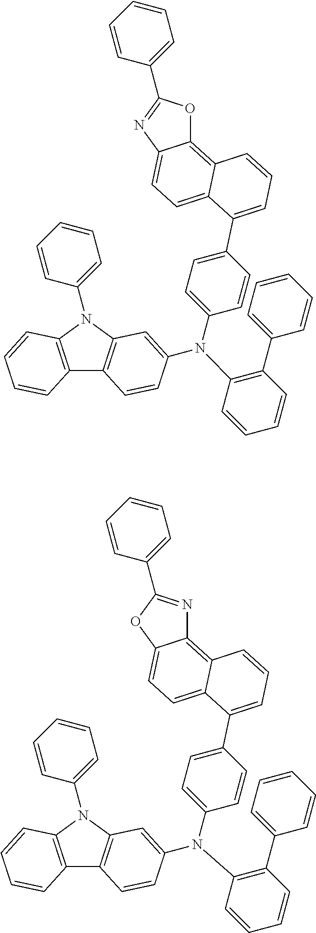 [SMILES: c1ccc(-c2nc3ccc4c(-c5ccc(N(c6ccc7c8ccccc8n(-c8ccccc8)c7c6)c6ccccc6-c6ccccc6)cc5)cccc4c3o2)cc1 ; c1ccc(-c2nc3c(ccc4c(-c5ccc(N(c6ccc7c8ccccc8n(-c8ccccc8)c7c6)c6ccccc6-c6ccccc6)cc5)cccc43)o2)cc1]

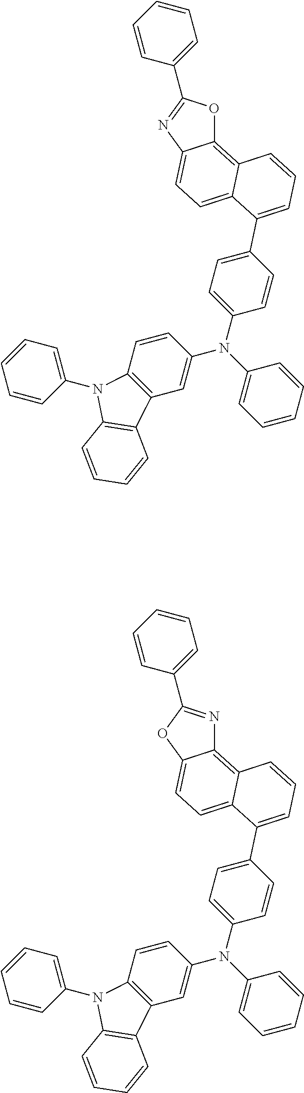 [SMILES: c1ccc(-c2nc3ccc4c(-c5ccc(N(c6ccccc6)c6ccc7c(c6)c6ccccc6n7-c6ccccc6)cc5)cccc4c3o2)cc1 ; c1ccc(-c2nc3c(ccc4c(-c5ccc(N(c6ccccc6)c6ccc7c(c6)c6ccccc6n7-c6ccccc6)cc5)cccc43)o2)cc1]

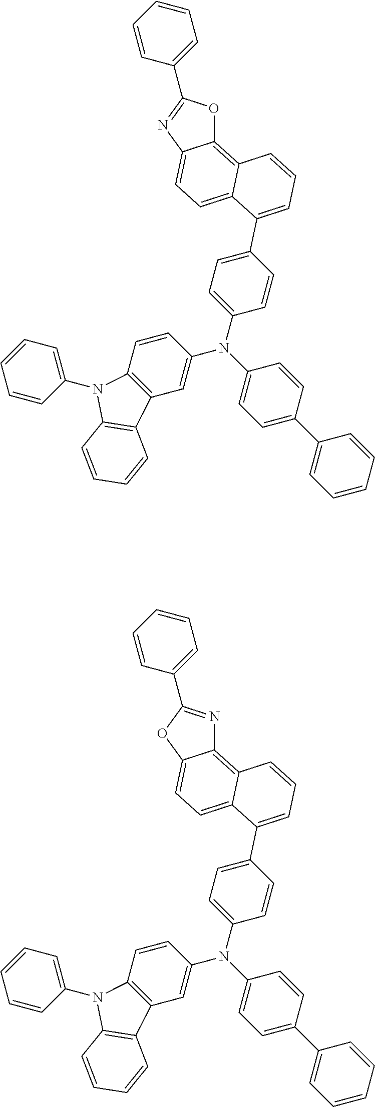 [SMILES: c1ccc(-c2ccc(N(c3ccc(-c4cccc5c4ccc4nc(-c6ccccc6)oc45)cc3)c3ccc4c(c3)c3ccccc3n4-c3ccccc3)cc2)cc1 ; c1ccc(-c2ccc(N(c3ccc(-c4cccc5c4ccc4oc(-c6ccccc6)nc45)cc3)c3ccc4c(c3)c3ccccc3n4-c3ccccc3)cc2)cc1]

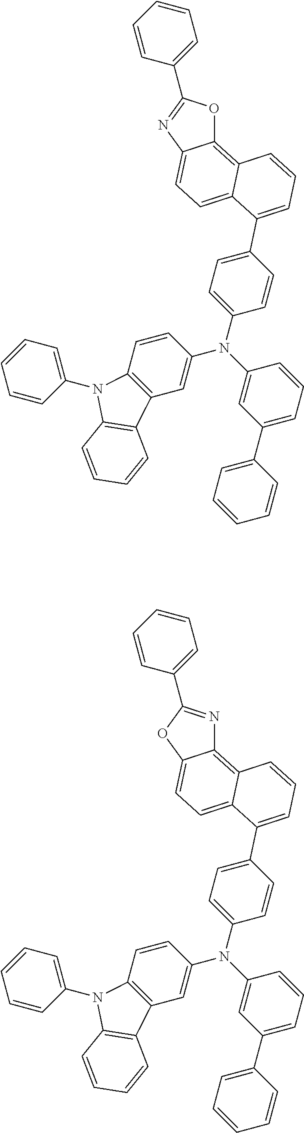 [SMILES: c1ccc(-c2cccc(N(c3ccc(-c4cccc5c4ccc4nc(-c6ccccc6)oc45)cc3)c3ccc4c(c3)c3ccccc3n4-c3ccccc3)c2)cc1 ; c1ccc(-c2cccc(N(c3ccc(-c4cccc5c4ccc4oc(-c6ccccc6)nc45)cc3)c3ccc4c(c3)c3ccccc3n4-c3ccccc3)c2)cc1]

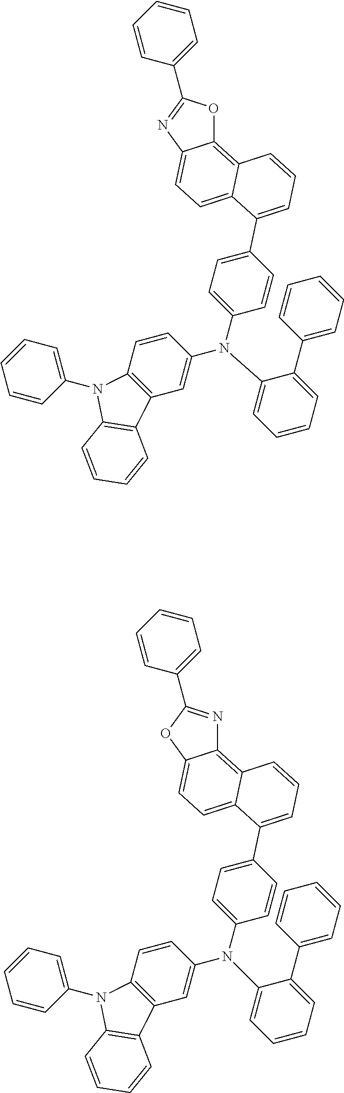 [SMILES: c1ccc(-c2nc3ccc4c(-c5ccc(N(c6ccc7c(c6)c6ccccc6n7-c6ccccc6)c6ccccc6-c6ccccc6)cc5)cccc4c3o2)cc1 ; c1ccc(-c2nc3c(ccc4c(-c5ccc(N(c6ccc7c(c6)c6ccccc6n7-c6ccccc6)c6ccccc6-c6ccccc6)cc5)cccc43)o2)cc1]

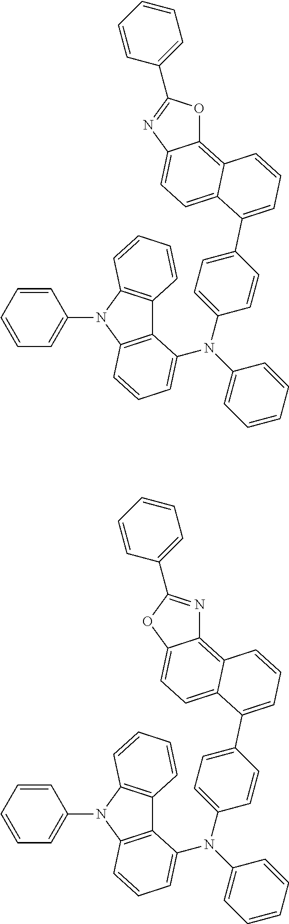 [SMILES: c1ccc(-c2nc3ccc4c(-c5ccc(N(c6ccccc6)c6cccc7c6c6ccccc6n7-c6ccccc6)cc5)cccc4c3o2)cc1 ; c1ccc(-c2nc3c(ccc4c(-c5ccc(N(c6ccccc6)c6cccc7c6c6ccccc6n7-c6ccccc6)cc5)cccc43)o2)cc1]

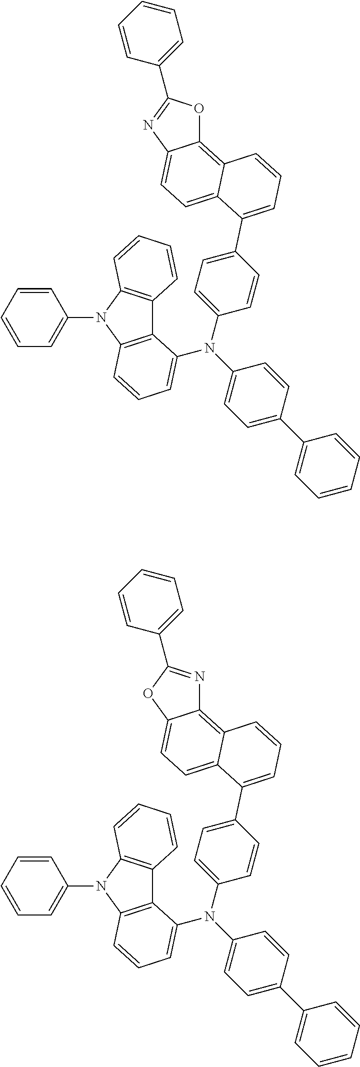 [SMILES: c1ccc(-c2ccc(N(c3ccc(-c4cccc5c4ccc4nc(-c6ccccc6)oc45)cc3)c3cccc4c3c3ccccc3n4-c3ccccc3)cc2)cc1 ; c1ccc(-c2ccc(N(c3ccc(-c4cccc5c4ccc4oc(-c6ccccc6)nc45)cc3)c3cccc4c3c3ccccc3n4-c3ccccc3)cc2)cc1]

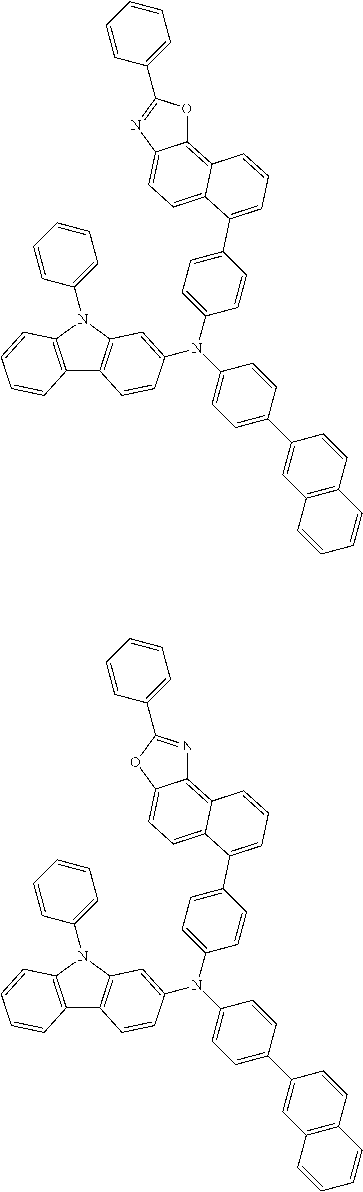 [SMILES: c1ccc(-c2nc3ccc4c(-c5ccc(N(c6ccc(-c7ccc8ccccc8c7)cc6)c6ccc7c8ccccc8n(-c8ccccc8)c7c6)cc5)cccc4c3o2)cc1 ; c1ccc(-c2nc3c(ccc4c(-c5ccc(N(c6ccc(-c7ccc8ccccc8c7)cc6)c6ccc7c8ccccc8n(-c8ccccc8)c7c6)cc5)cccc43)o2)cc1]

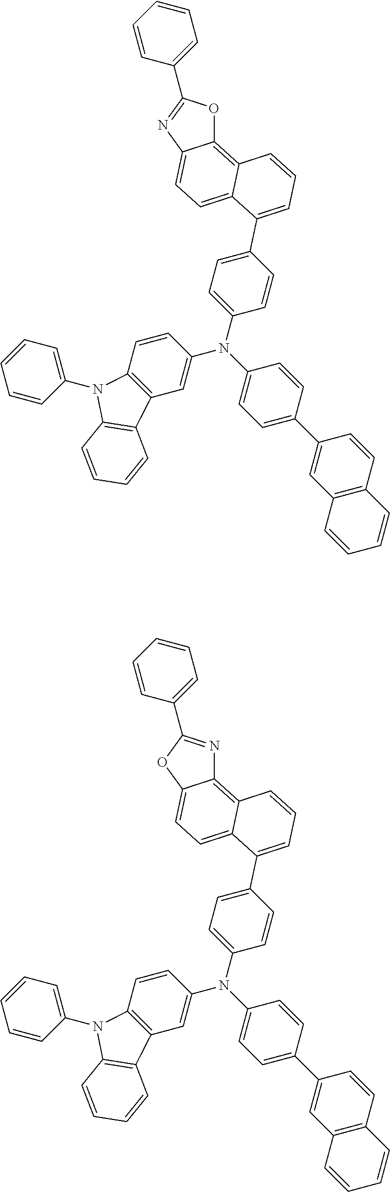 [SMILES: c1ccc(-c2nc3ccc4c(-c5ccc(N(c6ccc(-c7ccc8ccccc8c7)cc6)c6ccc7c(c6)c6ccccc6n7-c6ccccc6)cc5)cccc4c3o2)cc1 ; c1ccc(-c2nc3c(ccc4c(-c5ccc(N(c6ccc(-c7ccc8ccccc8c7)cc6)c6ccc7c(c6)c6ccccc6n7-c6ccccc6)cc5)cccc43)o2)cc1]

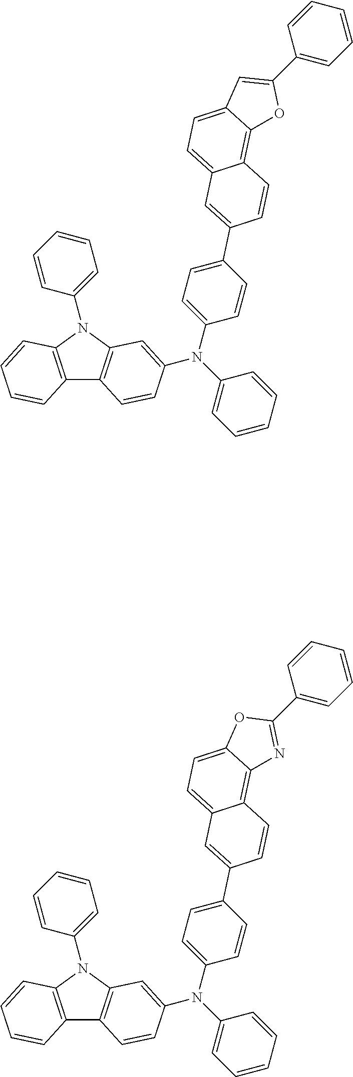 [SMILES: c1ccc(-c2cc3ccc4cc(-c5ccc(N(c6ccccc6)c6ccc7c8ccccc8n(-c8ccccc8)c7c6)cc5)ccc4c3o2)cc1 ; c1ccc(-c2nc3c(ccc4cc(-c5ccc(N(c6ccccc6)c6ccc7c8ccccc8n(-c8ccccc8)c7c6)cc5)ccc43)o2)cc1]

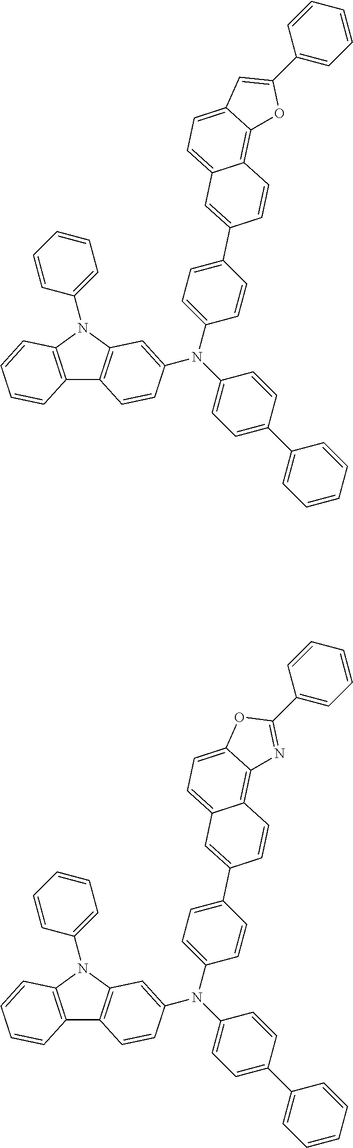 [SMILES: c1ccc(-c2ccc(N(c3ccc(-c4ccc5c(ccc6cc(-c7ccccc7)oc65)c4)cc3)c3ccc4c5ccccc5n(-c5ccccc5)c4c3)cc2)cc1 ; c1ccc(-c2ccc(N(c3ccc(-c4ccc5c(ccc6oc(-c7ccccc7)nc65)c4)cc3)c3ccc4c5ccccc5n(-c5ccccc5)c4c3)cc2)cc1]

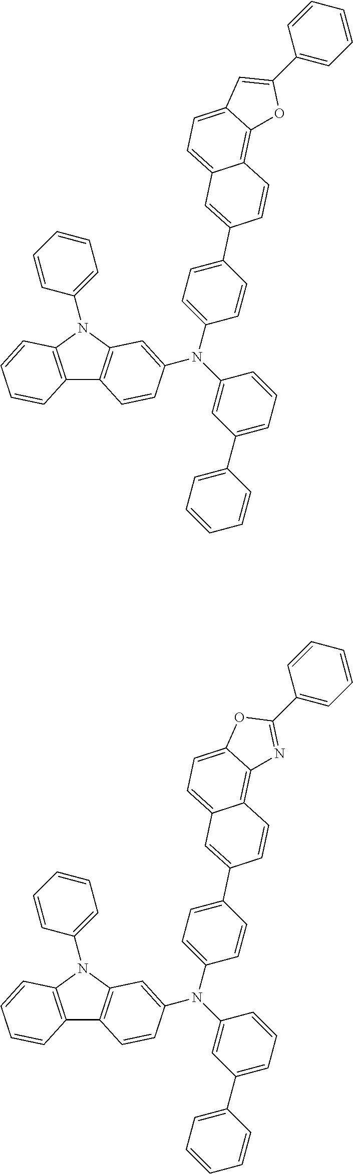 [SMILES: c1ccc(-c2cccc(N(c3ccc(-c4ccc5c(ccc6cc(-c7ccccc7)oc65)c4)cc3)c3ccc4c5ccccc5n(-c5ccccc5)c4c3)c2)cc1 ; c1ccc(-c2cccc(N(c3ccc(-c4ccc5c(ccc6oc(-c7ccccc7)nc65)c4)cc3)c3ccc4c5ccccc5n(-c5ccccc5)c4c3)c2)cc1]

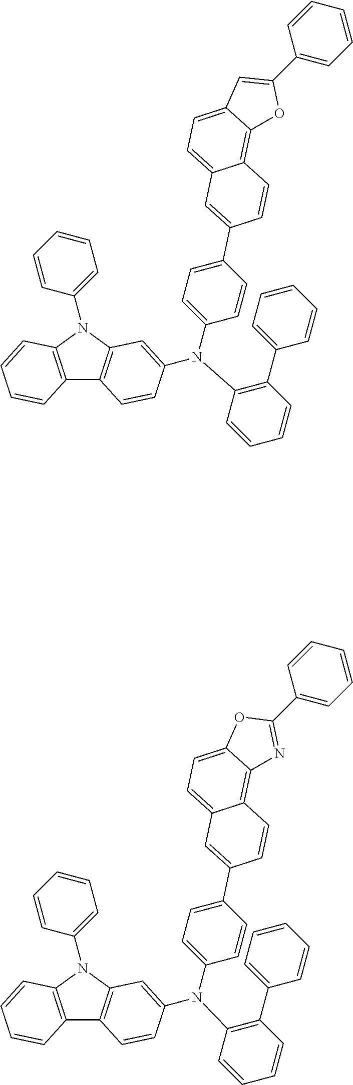 [SMILES: c1ccc(-c2cc3ccc4cc(-c5ccc(N(c6ccc7c8ccccc8n(-c8ccccc8)c7c6)c6ccccc6-c6ccccc6)cc5)ccc4c3o2)cc1 ; c1ccc(-c2nc3c(ccc4cc(-c5ccc(N(c6ccc7c8ccccc8n(-c8ccccc8)c7c6)c6ccccc6-c6ccccc6)cc5)ccc43)o2)cc1]

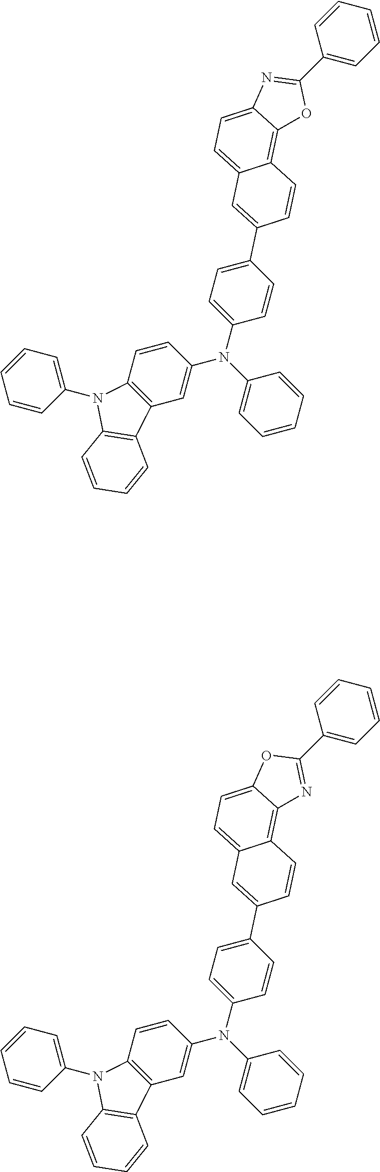 [SMILES: c1ccc(-c2nc3ccc4cc(-c5ccc(N(c6ccccc6)c6ccc7c(c6)c6ccccc6n7-c6ccccc6)cc5)ccc4c3o2)cc1 ; c1ccc(-c2nc3c(ccc4cc(-c5ccc(N(c6ccccc6)c6ccc7c(c6)c6ccccc6n7-c6ccccc6)cc5)ccc43)o2)cc1]

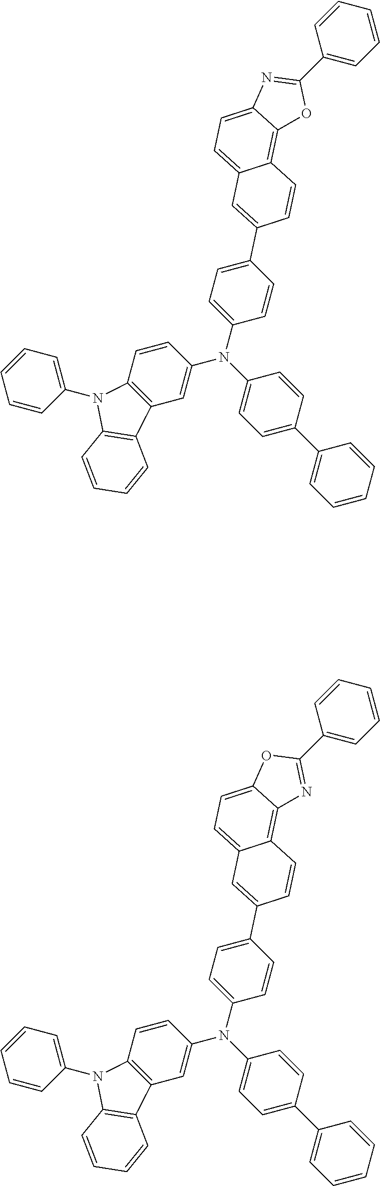 [SMILES: c1ccc(-c2ccc(N(c3ccc(-c4ccc5c(ccc6nc(-c7ccccc7)oc65)c4)cc3)c3ccc4c(c3)c3ccccc3n4-c3ccccc3)cc2)cc1 ; c1ccc(-c2ccc(N(c3ccc(-c4ccc5c(ccc6oc(-c7ccccc7)nc65)c4)cc3)c3ccc4c(c3)c3ccccc3n4-c3ccccc3)cc2)cc1]

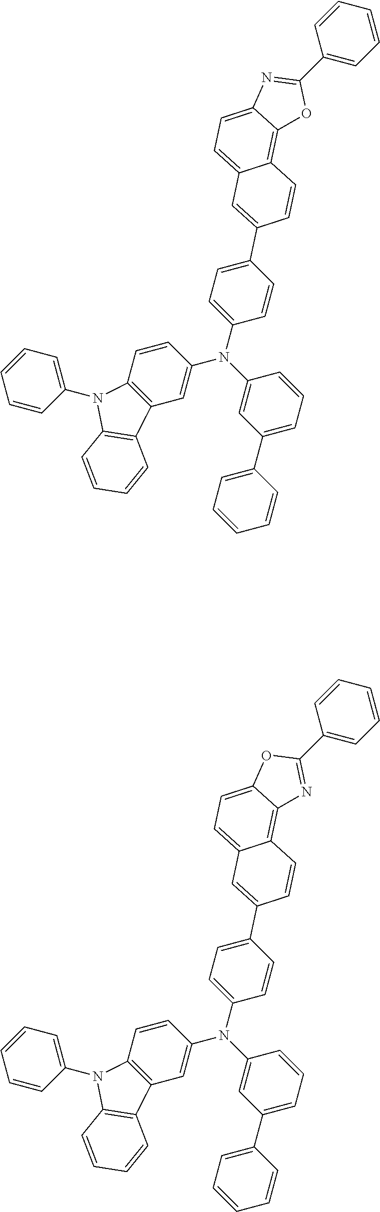 [SMILES: c1ccc(-c2cccc(N(c3ccc(-c4ccc5c(ccc6nc(-c7ccccc7)oc65)c4)cc3)c3ccc4c(c3)c3ccccc3n4-c3ccccc3)c2)cc1 ; c1ccc(-c2cccc(N(c3ccc(-c4ccc5c(ccc6oc(-c7ccccc7)nc65)c4)cc3)c3ccc4c(c3)c3ccccc3n4-c3ccccc3)c2)cc1]

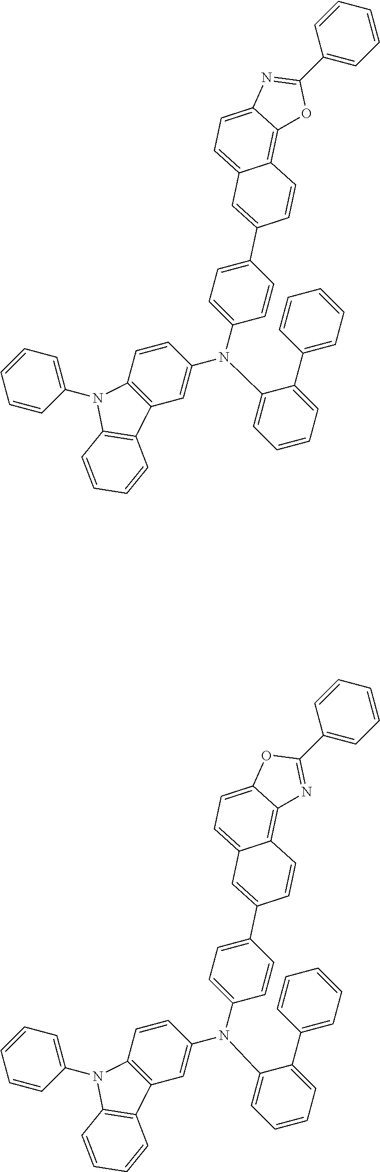 [SMILES: c1ccc(-c2nc3ccc4cc(-c5ccc(N(c6ccc7c(c6)c6ccccc6n7-c6ccccc6)c6ccccc6-c6ccccc6)cc5)ccc4c3o2)cc1 ; c1ccc(-c2nc3c(ccc4cc(-c5ccc(N(c6ccc7c(c6)c6ccccc6n7-c6ccccc6)c6ccccc6-c6ccccc6)cc5)ccc43)o2)cc1]

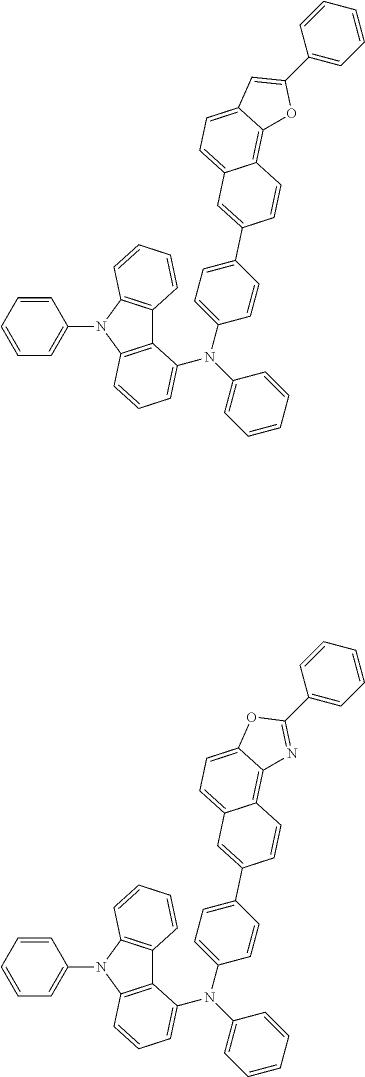 [SMILES: c1ccc(-c2cc3ccc4cc(-c5ccc(N(c6ccccc6)c6cccc7c6c6ccccc6n7-c6ccccc6)cc5)ccc4c3o2)cc1 ; c1ccc(-c2nc3c(ccc4cc(-c5ccc(N(c6ccccc6)c6cccc7c6c6ccccc6n7-c6ccccc6)cc5)ccc43)o2)cc1]

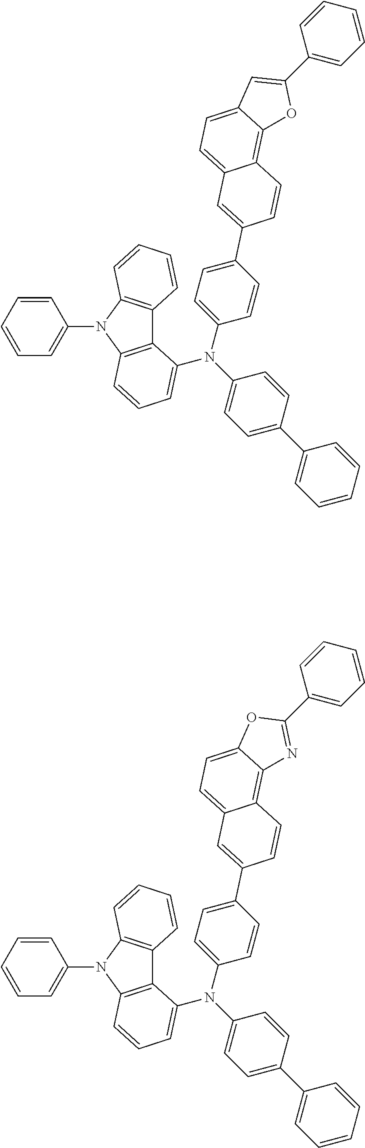 [SMILES: c1ccc(-c2ccc(N(c3ccc(-c4ccc5c(ccc6cc(-c7ccccc7)oc65)c4)cc3)c3cccc4c3c3ccccc3n4-c3ccccc3)cc2)cc1 ; c1ccc(-c2ccc(N(c3ccc(-c4ccc5c(ccc6oc(-c7ccccc7)nc65)c4)cc3)c3cccc4c3c3ccccc3n4-c3ccccc3)cc2)cc1]

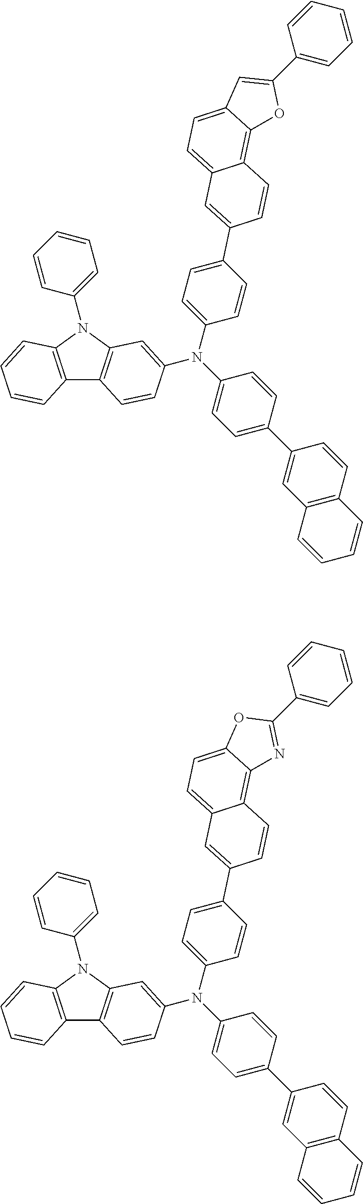 [SMILES: c1ccc(-c2cc3ccc4cc(-c5ccc(N(c6ccc(-c7ccc8ccccc8c7)cc6)c6ccc7c8ccccc8n(-c8ccccc8)c7c6)cc5)ccc4c3o2)cc1 ; c1ccc(-c2nc3c(ccc4cc(-c5ccc(N(c6ccc(-c7ccc8ccccc8c7)cc6)c6ccc7c8ccccc8n(-c8ccccc8)c7c6)cc5)ccc43)o2)cc1]

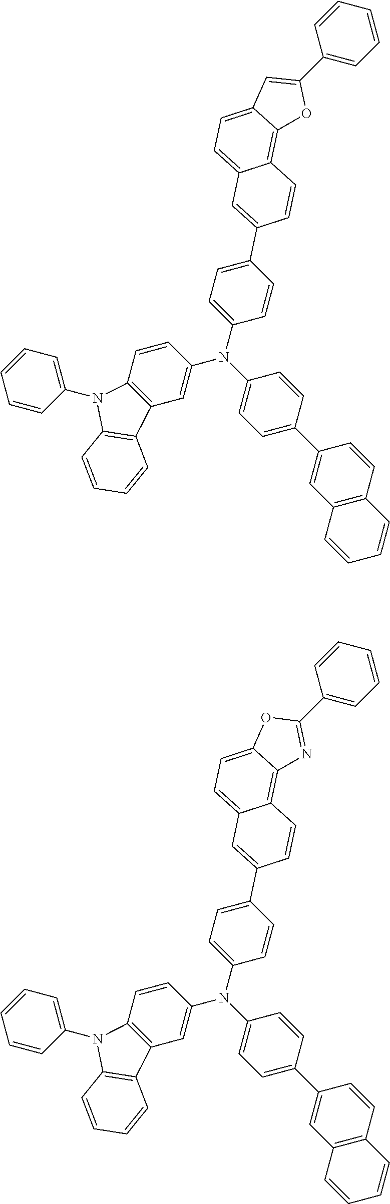 [SMILES: c1ccc(-c2cc3ccc4cc(-c5ccc(N(c6ccc(-c7ccc8ccccc8c7)cc6)c6ccc7c(c6)c6ccccc6n7-c6ccccc6)cc5)ccc4c3o2)cc1 ; c1ccc(-c2nc3c(ccc4cc(-c5ccc(N(c6ccc(-c7ccc8ccccc8c7)cc6)c6ccc7c(c6)c6ccccc6n7-c6ccccc6)cc5)ccc43)o2)cc1]

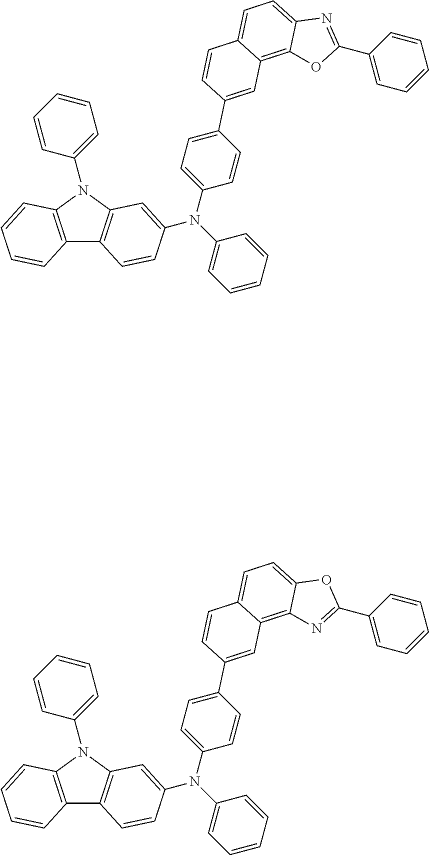 [SMILES: c1ccc(-c2nc3ccc4ccc(-c5ccc(N(c6ccccc6)c6ccc7c8ccccc8n(-c8ccccc8)c7c6)cc5)cc4c3o2)cc1 ; c1ccc(-c2nc3c(ccc4ccc(-c5ccc(N(c6ccccc6)c6ccc7c8ccccc8n(-c8ccccc8)c7c6)cc5)cc43)o2)cc1]

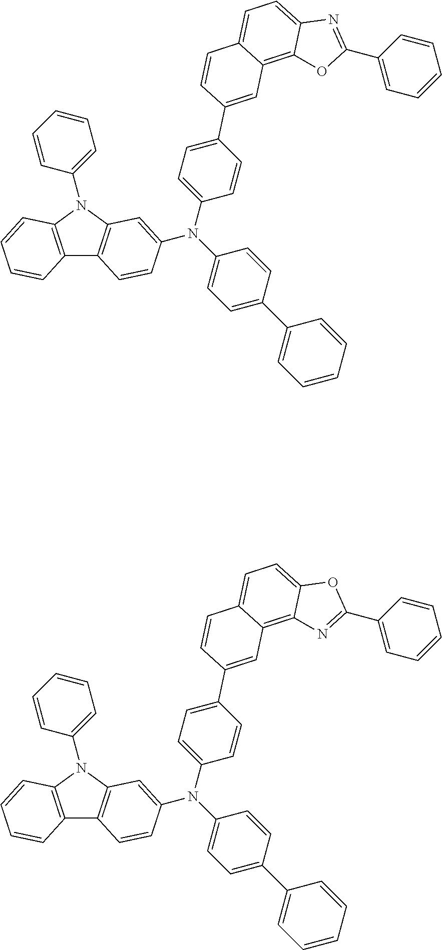 [SMILES: c1ccc(-c2ccc(N(c3ccc(-c4ccc5ccc6nc(-c7ccccc7)oc6c5c4)cc3)c3ccc4c5ccccc5n(-c5ccccc5)c4c3)cc2)cc1 ; c1ccc(-c2ccc(N(c3ccc(-c4ccc5ccc6oc(-c7ccccc7)nc6c5c4)cc3)c3ccc4c5ccccc5n(-c5ccccc5)c4c3)cc2)cc1]

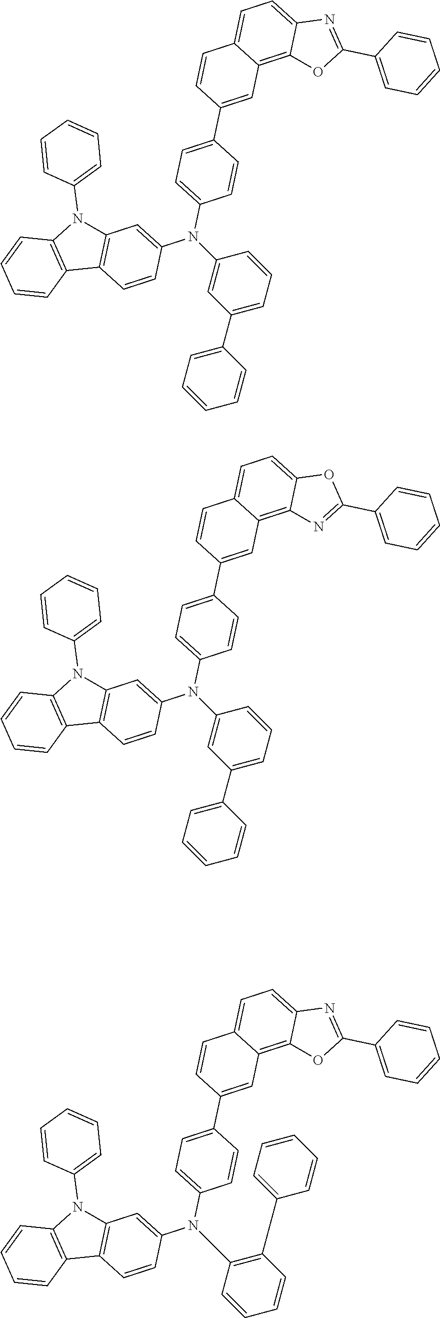 [SMILES: c1ccc(-c2cccc(N(c3ccc(-c4ccc5ccc6nc(-c7ccccc7)oc6c5c4)cc3)c3ccc4c5ccccc5n(-c5ccccc5)c4c3)c2)cc1 ; c1ccc(-c2cccc(N(c3ccc(-c4ccc5ccc6oc(-c7ccccc7)nc6c5c4)cc3)c3ccc4c5ccccc5n(-c5ccccc5)c4c3)c2)cc1 ; c1ccc(-c2nc3ccc4ccc(-c5ccc(N(c6ccc7c8ccccc8n(-c8ccccc8)c7c6)c6ccccc6-c6ccccc6)cc5)cc4c3o2)cc1]

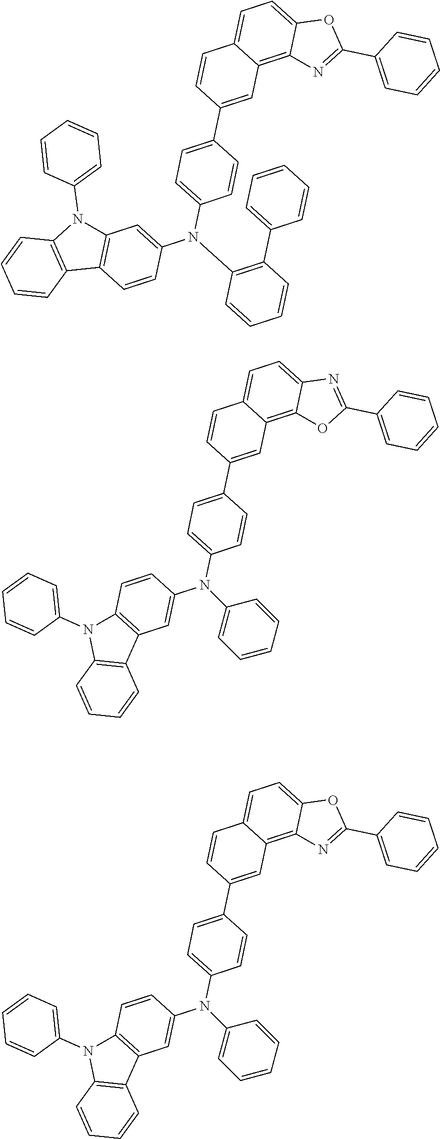 [SMILES: c1ccc(-c2nc3c(ccc4ccc(-c5ccc(N(c6ccc7c8ccccc8n(-c8ccccc8)c7c6)c6ccccc6-c6ccccc6)cc5)cc43)o2)cc1 ; c1ccc(-c2nc3ccc4ccc(-c5ccc(N(c6ccccc6)c6ccc7c(c6)c6ccccc6n7-c6ccccc6)cc5)cc4c3o2)cc1 ; c1ccc(-c2nc3c(ccc4ccc(-c5ccc(N(c6ccccc6)c6ccc7c(c6)c6ccccc6n7-c6ccccc6)cc5)cc43)o2)cc1]

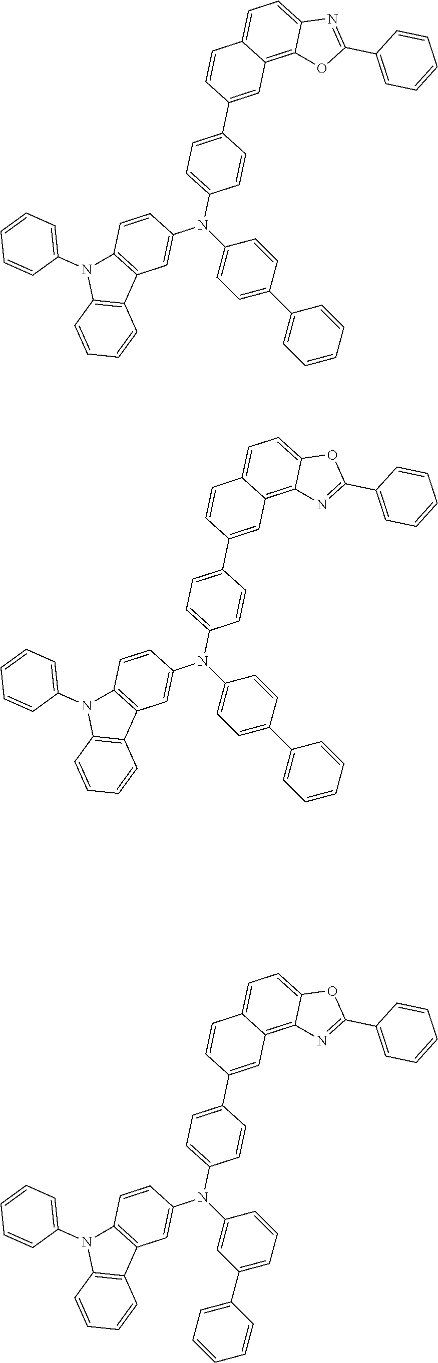 [SMILES: c1ccc(-c2ccc(N(c3ccc(-c4ccc5ccc6nc(-c7ccccc7)oc6c5c4)cc3)c3ccc4c(c3)c3ccccc3n4-c3ccccc3)cc2)cc1 ; c1ccc(-c2ccc(N(c3ccc(-c4ccc5ccc6oc(-c7ccccc7)nc6c5c4)cc3)c3ccc4c(c3)c3ccccc3n4-c3ccccc3)cc2)cc1 ; c1ccc(-c2cccc(N(c3ccc(-c4ccc5ccc6oc(-c7ccccc7)nc6c5c4)cc3)c3ccc4c(c3)c3ccccc3n4-c3ccccc3)c2)cc1]

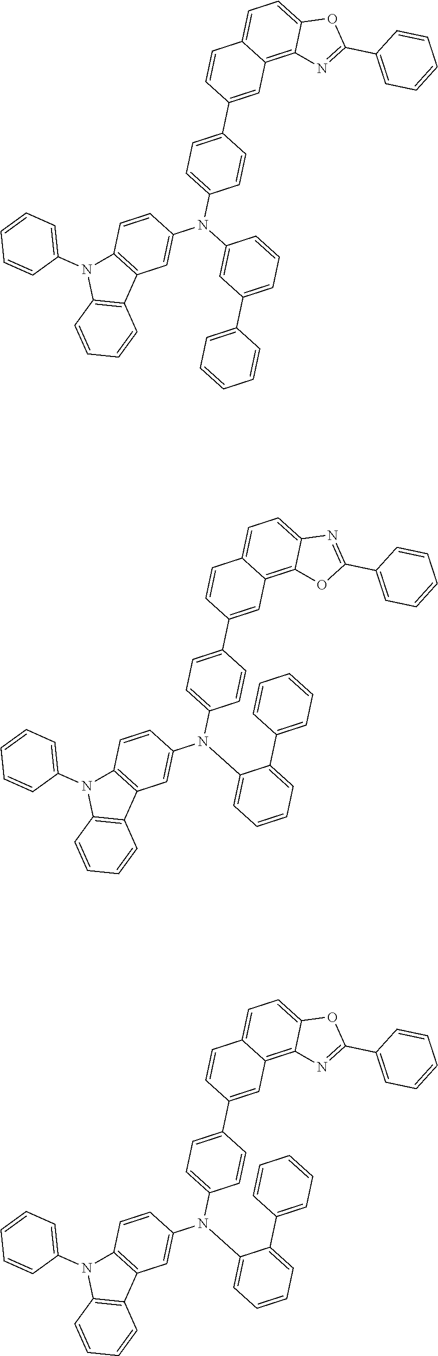 [SMILES: c1ccc(-c2cccc(N(c3ccc(-c4ccc5ccc6oc(-c7ccccc7)nc6c5c4)cc3)c3ccc4c(c3)c3ccccc3n4-c3ccccc3)c2)cc1 ; c1ccc(-c2nc3ccc4ccc(-c5ccc(N(c6ccc7c(c6)c6ccccc6n7-c6ccccc6)c6ccccc6-c6ccccc6)cc5)cc4c3o2)cc1 ; c1ccc(-c2nc3c(ccc4ccc(-c5ccc(N(c6ccc7c(c6)c6ccccc6n7-c6ccccc6)c6ccccc6-c6ccccc6)cc5)cc43)o2)cc1]

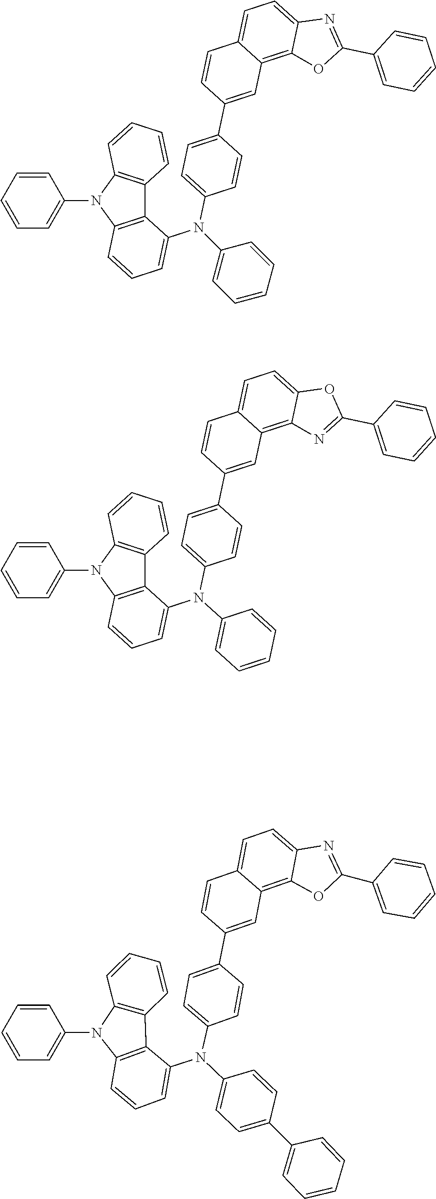 [SMILES: c1ccc(-c2nc3ccc4ccc(-c5ccc(N(c6ccccc6)c6cccc7c6c6ccccc6n7-c6ccccc6)cc5)cc4c3o2)cc1 ; c1ccc(-c2nc3c(ccc4ccc(-c5ccc(N(c6ccccc6)c6cccc7c6c6ccccc6n7-c6ccccc6)cc5)cc43)o2)cc1 ; c1ccc(-c2ccc(N(c3ccc(-c4ccc5ccc6nc(-c7ccccc7)oc6c5c4)cc3)c3cccc4c3c3ccccc3n4-c3ccccc3)cc2)cc1]

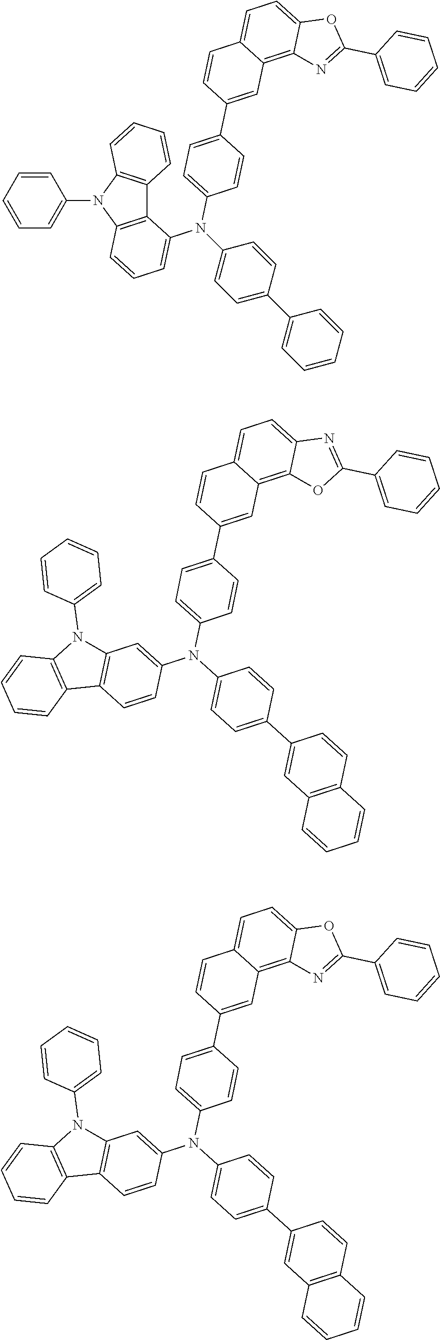 [SMILES: c1ccc(-c2ccc(N(c3ccc(-c4ccc5ccc6oc(-c7ccccc7)nc6c5c4)cc3)c3cccc4c3c3ccccc3n4-c3ccccc3)cc2)cc1 ; c1ccc(-c2nc3ccc4ccc(-c5ccc(N(c6ccc(-c7ccc8ccccc8c7)cc6)c6ccc7c8ccccc8n(-c8ccccc8)c7c6)cc5)cc4c3o2)cc1 ; c1ccc(-c2nc3c(ccc4ccc(-c5ccc(N(c6ccc(-c7ccc8ccccc8c7)cc6)c6ccc7c8ccccc8n(-c8ccccc8)c7c6)cc5)cc43)o2)cc1]

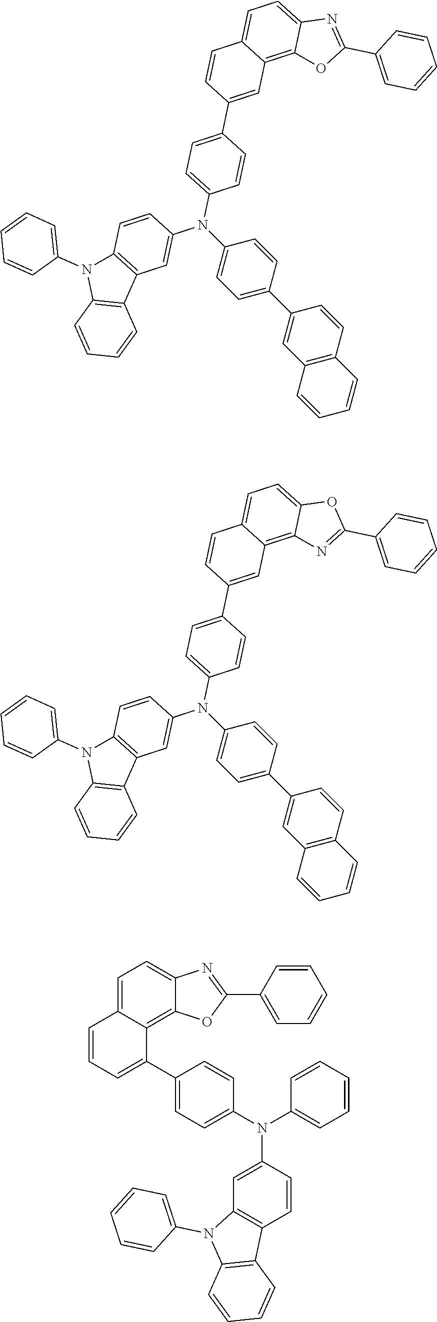 [SMILES: c1ccc(-c2nc3ccc4ccc(-c5ccc(N(c6ccc(-c7ccc8ccccc8c7)cc6)c6ccc7c(c6)c6ccccc6n7-c6ccccc6)cc5)cc4c3o2)cc1 ; c1ccc(-c2nc3c(ccc4ccc(-c5ccc(N(c6ccc(-c7ccc8ccccc8c7)cc6)c6ccc7c(c6)c6ccccc6n7-c6ccccc6)cc5)cc43)o2)cc1 ; c1ccc(-c2nc3ccc4cccc(-c5ccc(N(c6ccccc6)c6ccc7c8ccccc8n(-c8ccccc8)c7c6)cc5)c4c3o2)cc1]

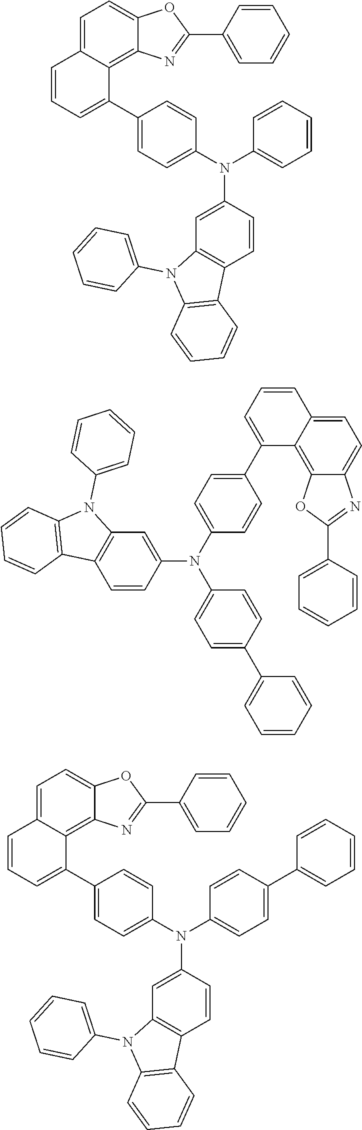 [SMILES: c1ccc(-c2nc3c(ccc4cccc(-c5ccc(N(c6ccccc6)c6ccc7c8ccccc8n(-c8ccccc8)c7c6)cc5)c43)o2)cc1 ; c1ccc(-c2ccc(N(c3ccc(-c4cccc5ccc6nc(-c7ccccc7)oc6c45)cc3)c3ccc4c5ccccc5n(-c5ccccc5)c4c3)cc2)cc1 ; c1ccc(-c2ccc(N(c3ccc(-c4cccc5ccc6oc(-c7ccccc7)nc6c45)cc3)c3ccc4c5ccccc5n(-c5ccccc5)c4c3)cc2)cc1]

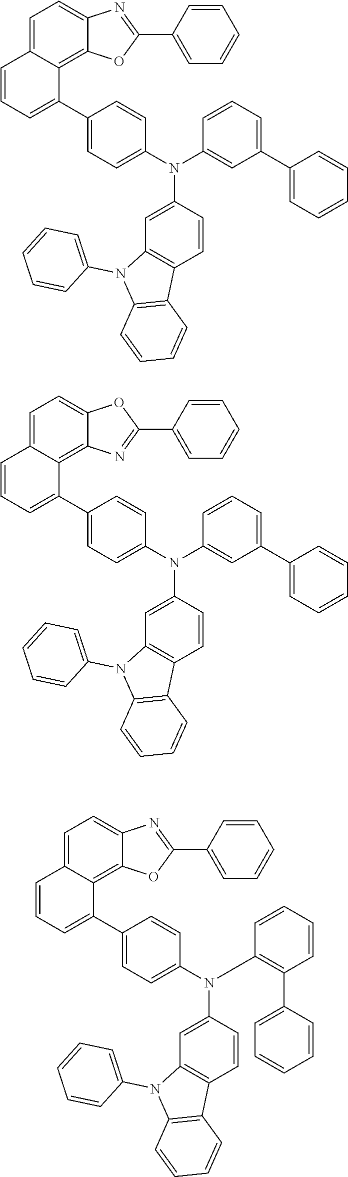

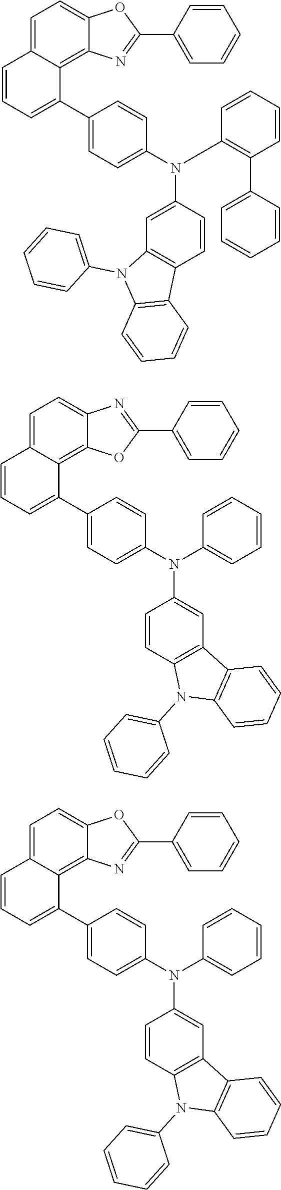

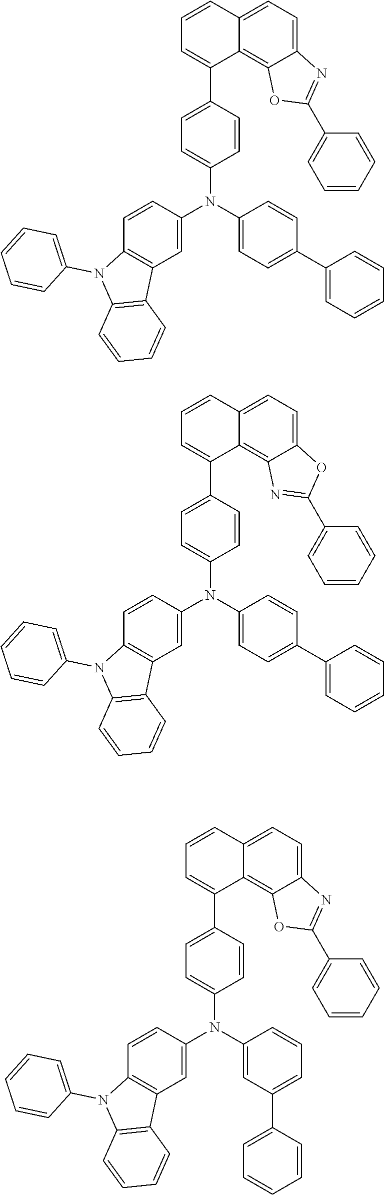 [SMILES: c1ccc(-c2ccc(N(c3ccc(-c4cccc5ccc6nc(-c7ccccc7)oc6c45)cc3)c3ccc4c(c3)c3ccccc3n4-c3ccccc3)cc2)cc1 ; c1ccc(-c2ccc(N(c3ccc(-c4cccc5ccc6oc(-c7ccccc7)nc6c45)cc3)c3ccc4c(c3)c3ccccc3n4-c3ccccc3)cc2)cc1 ; c1ccc(-c2cccc(N(c3ccc(-c4cccc5ccc6nc(-c7ccccc7)oc6c45)cc3)c3ccc4c(c3)c3ccccc3n4-c3ccccc3)c2)cc1]

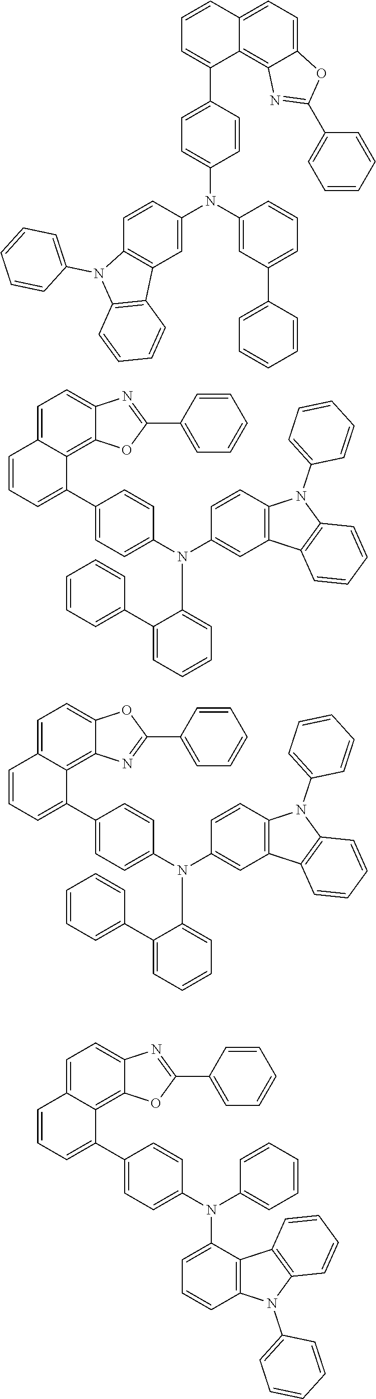

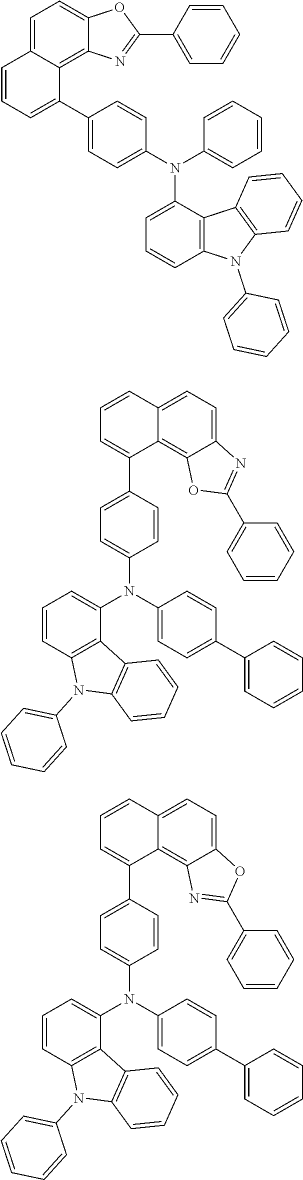 [SMILES: c1ccc(-c2nc3c(ccc4cccc(-c5ccc(N(c6ccccc6)c6cccc7c6c6ccccc6n7-c6ccccc6)cc5)c43)o2)cc1 ; c1ccc(-c2ccc(N(c3ccc(-c4cccc5ccc6nc(-c7ccccc7)oc6c45)cc3)c3cccc4c3c3ccccc3n4-c3ccccc3)cc2)cc1 ; c1ccc(-c2ccc(N(c3ccc(-c4cccc5ccc6oc(-c7ccccc7)nc6c45)cc3)c3cccc4c3c3ccccc3n4-c3ccccc3)cc2)cc1]

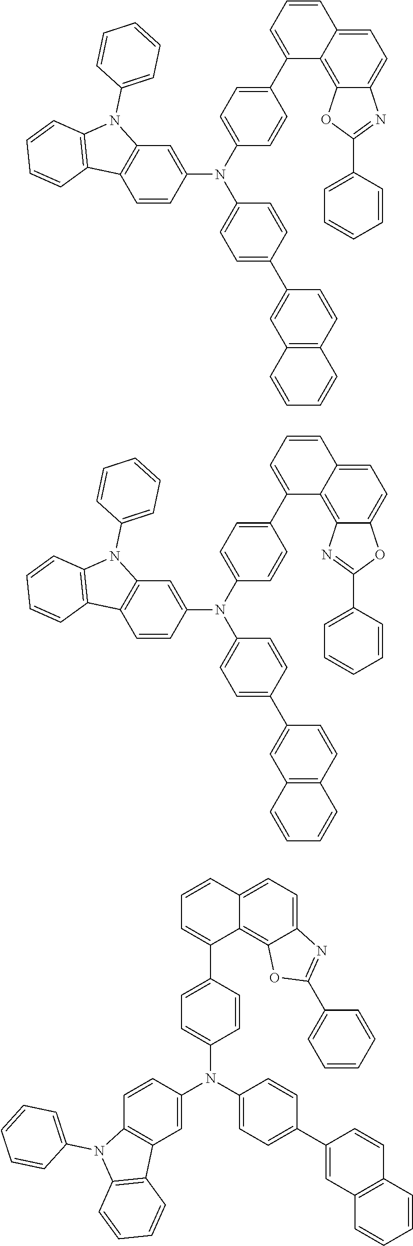

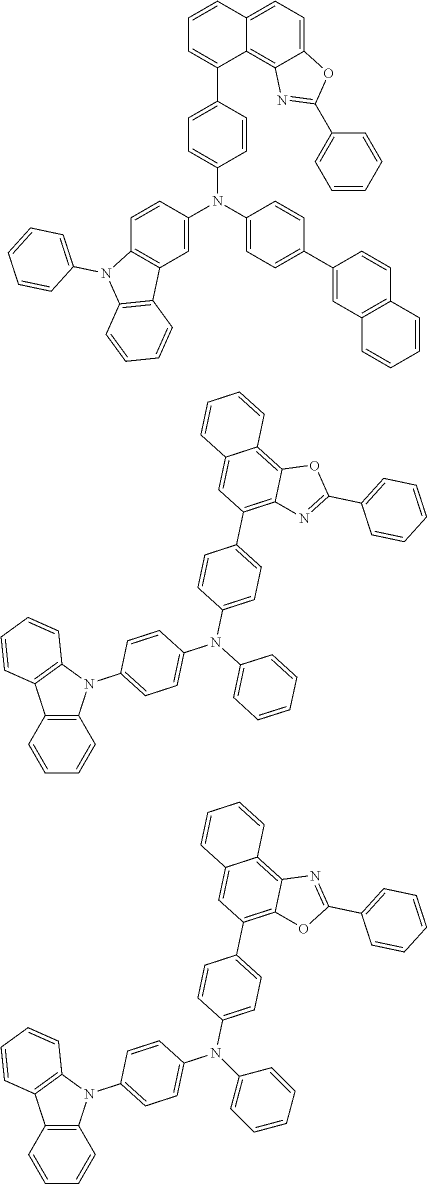 [SMILES: c1ccc(-c2nc3c(ccc4cccc(-c5ccc(N(c6ccc(-c7ccc8ccccc8c7)cc6)c6ccc7c(c6)c6ccccc6n7-c6ccccc6)cc5)c43)o2)cc1 ; c1ccc(-c2nc3c(-c4ccc(N(c5ccccc5)c5ccc(-n6c7ccccc7c7ccccc76)cc5)cc4)cc4ccccc4c3o2)cc1 ; c1ccc(-c2nc3c(o2)c(-c2ccc(N(c4ccccc4)c4ccc(-n5c6ccccc6c6ccccc65)cc4)cc2)cc2ccccc23)cc1]

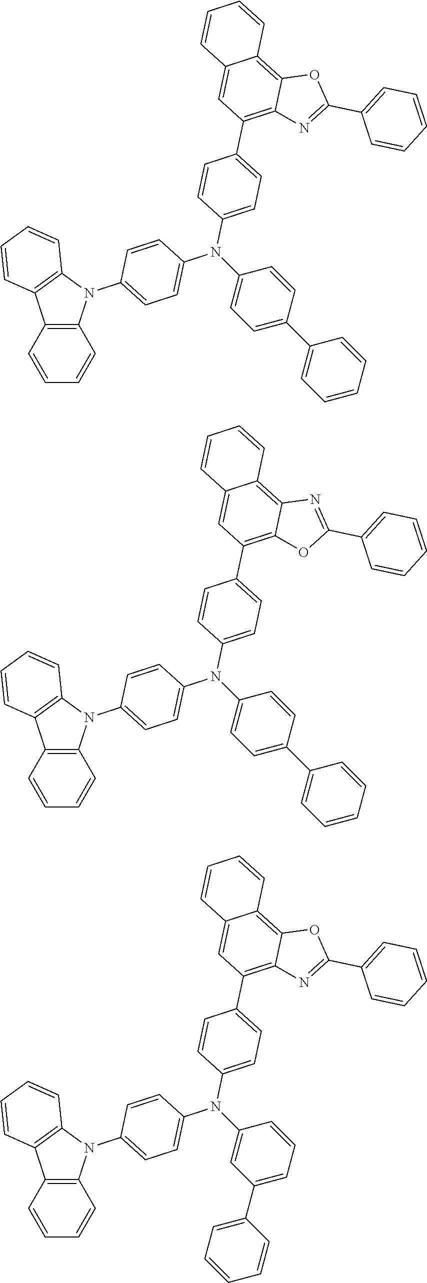 [SMILES: c1ccc(-c2ccc(N(c3ccc(-c4cc5ccccc5c5oc(-c6ccccc6)nc45)cc3)c3ccc(-n4c5ccccc5c5ccccc54)cc3)cc2)cc1 ; c1ccc(-c2ccc(N(c3ccc(-c4cc5ccccc5c5nc(-c6ccccc6)oc45)cc3)c3ccc(-n4c5ccccc5c5ccccc54)cc3)cc2)cc1 ; c1ccc(-c2cccc(N(c3ccc(-c4cc5ccccc5c5oc(-c6ccccc6)nc45)cc3)c3ccc(-n4c5ccccc5c5ccccc54)cc3)c2)cc1]

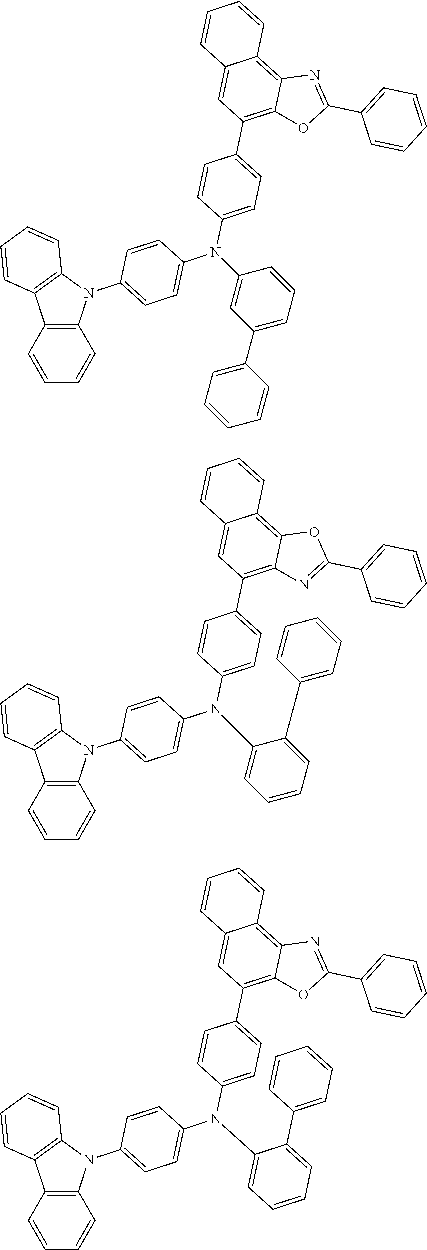

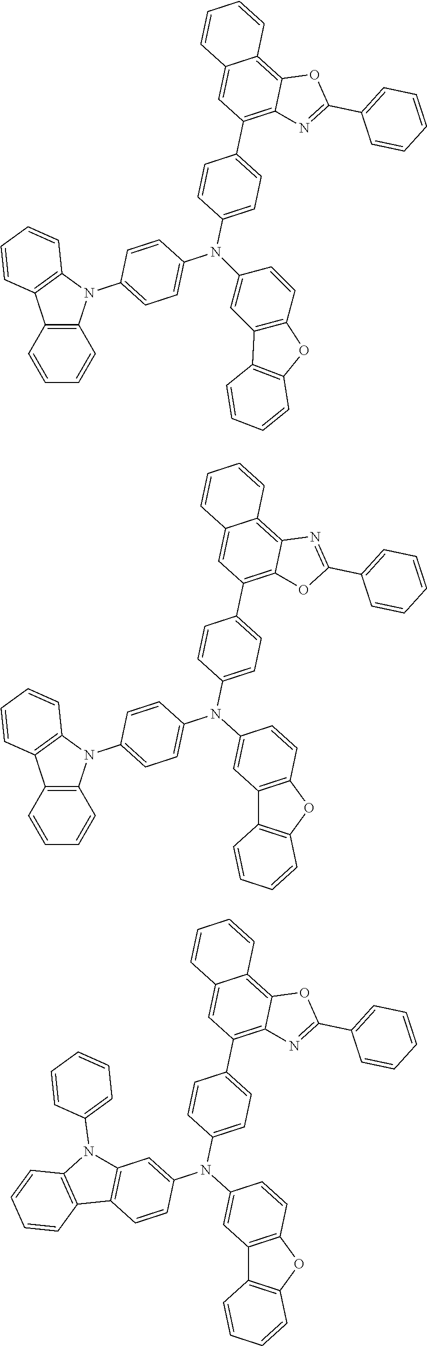

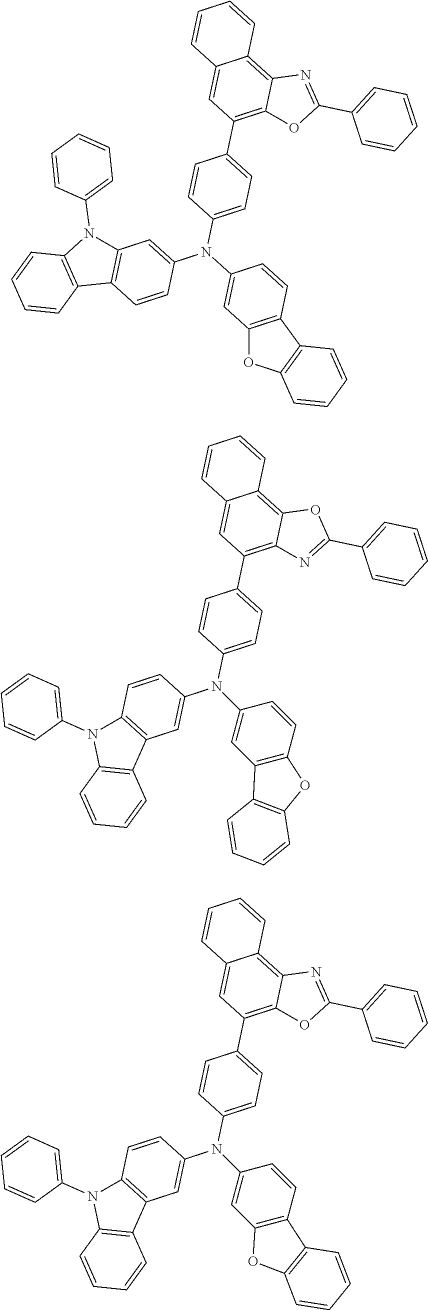

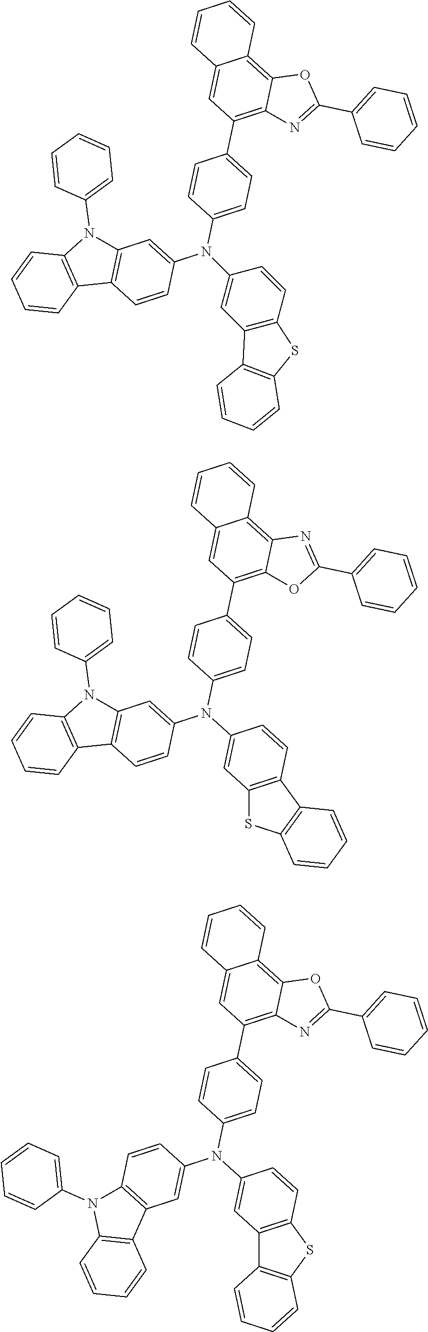

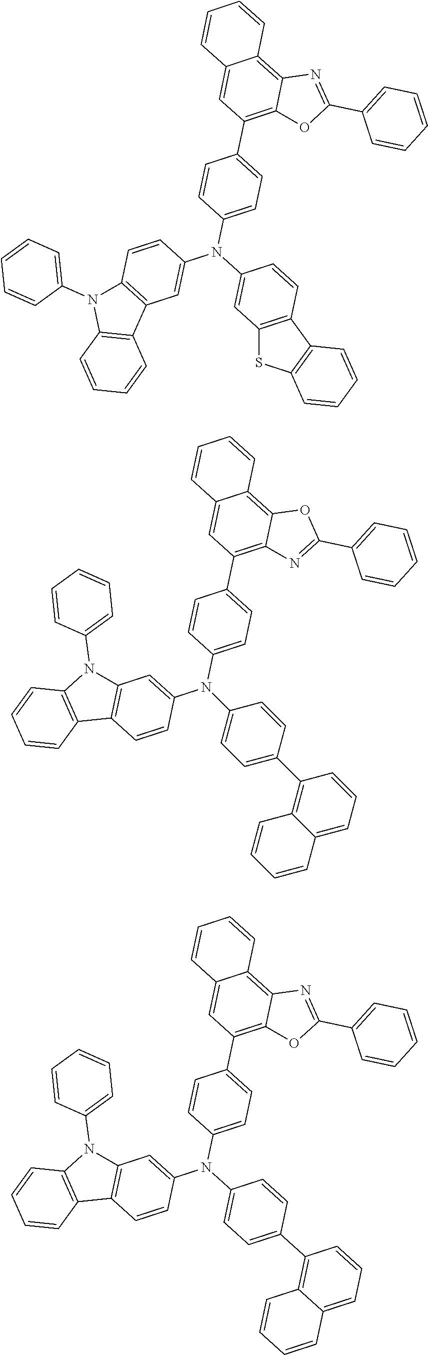 [SMILES: c1ccc(-c2nc3c(o2)c(-c2ccc(N(c4ccc5c(c4)sc4ccccc45)c4ccc5c(c4)c4ccccc4n5-c4ccccc4)cc2)cc2ccccc23)cc1 ; c1ccc(-c2nc3c(-c4ccc(N(c5ccc(-c6cccc7ccccc67)cc5)c5ccc6c7ccccc7n(-c7ccccc7)c6c5)cc4)cc4ccccc4c3o2)cc1 ; c1ccc(-c2nc3c(o2)c(-c2ccc(N(c4ccc(-c5cccc6ccccc56)cc4)c4ccc5c6ccccc6n(-c6ccccc6)c5c4)cc2)cc2ccccc23)cc1]

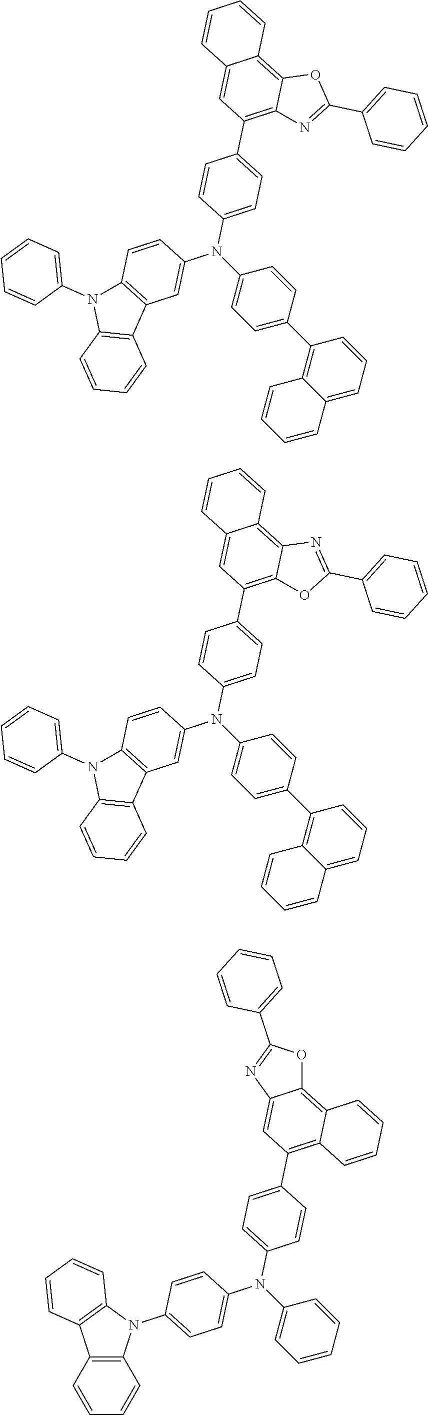 [SMILES: c1ccc(-c2nc3c(-c4ccc(N(c5ccc(-c6cccc7ccccc67)cc5)c5ccc6c(c5)c5ccccc5n6-c5ccccc5)cc4)cc4ccccc4c3o2)cc1 ; c1ccc(-c2nc3c(o2)c(-c2ccc(N(c4ccc(-c5cccc6ccccc56)cc4)c4ccc5c(c4)c4ccccc4n5-c4ccccc4)cc2)cc2ccccc23)cc1 ; c1ccc(-c2nc3cc(-c4ccc(N(c5ccccc5)c5ccc(-n6c7ccccc7c7ccccc76)cc5)cc4)c4ccccc4c3o2)cc1]

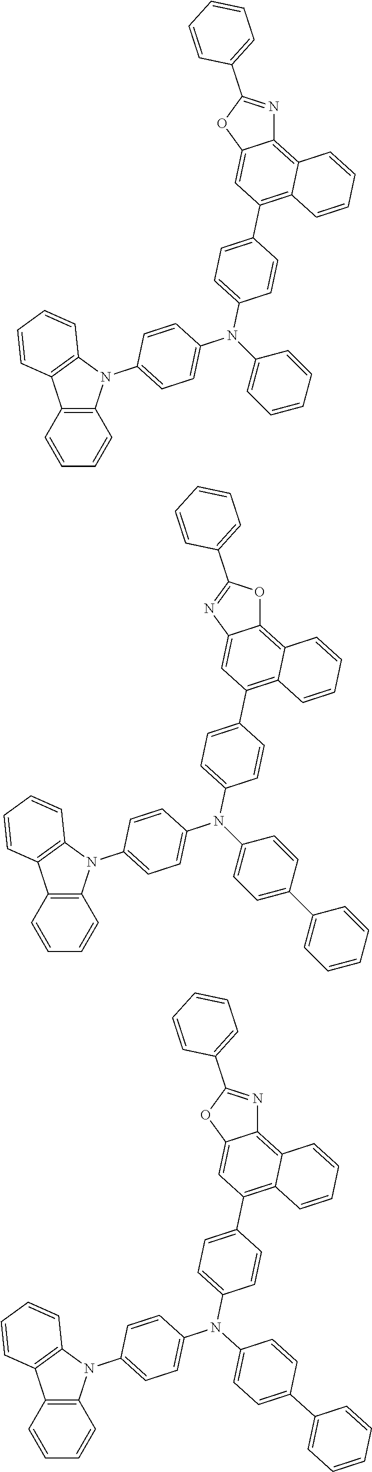 [SMILES: c1ccc(-c2nc3c(cc(-c4ccc(N(c5ccccc5)c5ccc(-n6c7ccccc7c7ccccc76)cc5)cc4)c4ccccc43)o2)cc1 ; c1ccc(-c2ccc(N(c3ccc(-c4cc5nc(-c6ccccc6)oc5c5ccccc45)cc3)c3ccc(-n4c5ccccc5c5ccccc54)cc3)cc2)cc1 ; c1ccc(-c2ccc(N(c3ccc(-c4cc5oc(-c6ccccc6)nc5c5ccccc45)cc3)c3ccc(-n4c5ccccc5c5ccccc54)cc3)cc2)cc1]

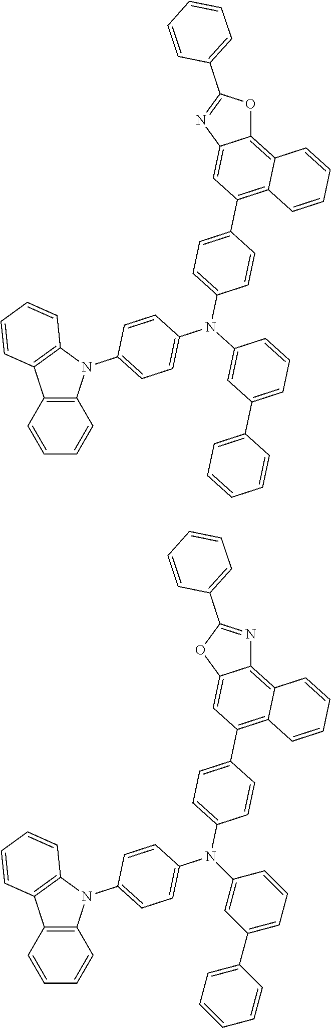

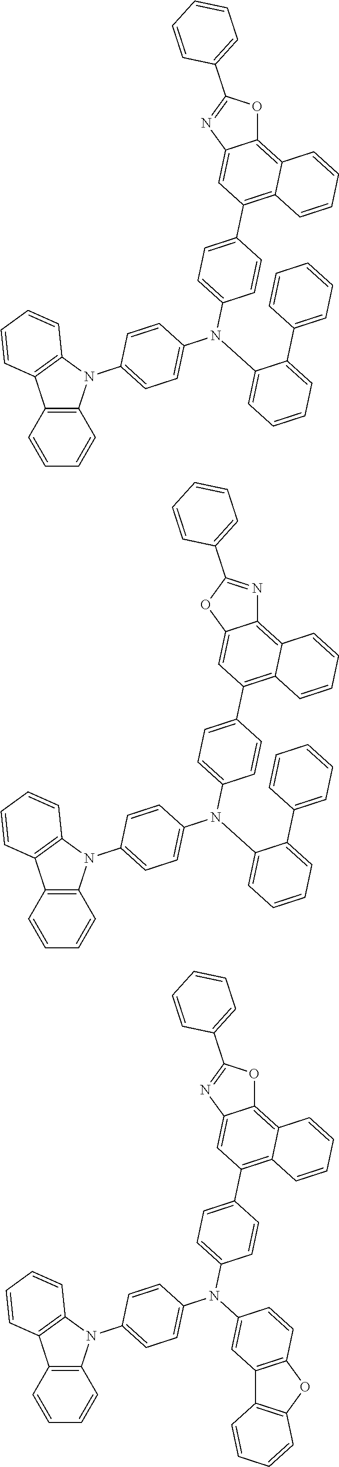 [SMILES: c1ccc(-c2nc3cc(-c4ccc(N(c5ccc(-n6c7ccccc7c7ccccc76)cc5)c5ccccc5-c5ccccc5)cc4)c4ccccc4c3o2)cc1 ; c1ccc(-c2nc3c(cc(-c4ccc(N(c5ccc(-n6c7ccccc7c7ccccc76)cc5)c5ccccc5-c5ccccc5)cc4)c4ccccc43)o2)cc1 ; c1ccc(-c2nc3cc(-c4ccc(N(c5ccc(-n6c7ccccc7c7ccccc76)cc5)c5ccc6oc7ccccc7c6c5)cc4)c4ccccc4c3o2)cc1]

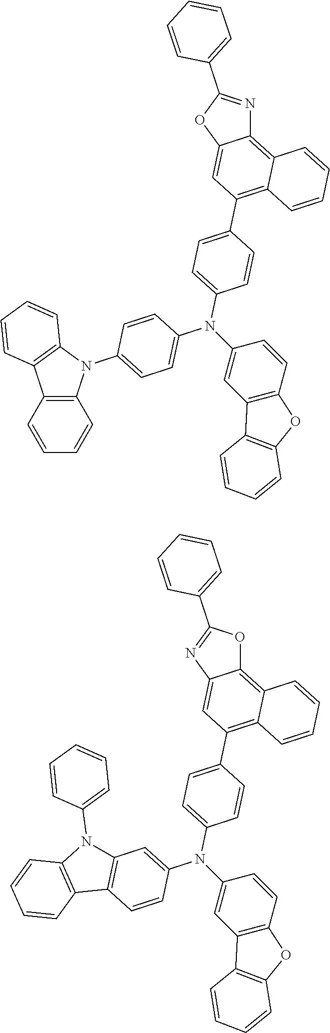 [SMILES: c1ccc(-c2nc3c(cc(-c4ccc(N(c5ccc(-n6c7ccccc7c7ccccc76)cc5)c5ccc6oc7ccccc7c6c5)cc4)c4ccccc43)o2)cc1 ; c1ccc(-c2nc3cc(-c4ccc(N(c5ccc6oc7ccccc7c6c5)c5ccc6c7ccccc7n(-c7ccccc7)c6c5)cc4)c4ccccc4c3o2)cc1]

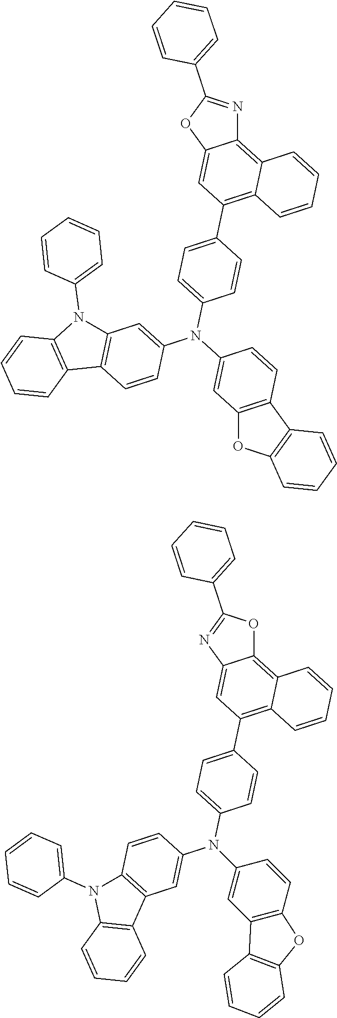 [SMILES: c1ccc(-c2nc3c(cc(-c4ccc(N(c5ccc6c(c5)oc5ccccc56)c5ccc6c7ccccc7n(-c7ccccc7)c6c5)cc4)c4ccccc43)o2)cc1 ; c1ccc(-c2nc3cc(-c4ccc(N(c5ccc6oc7ccccc7c6c5)c5ccc6c(c5)c5ccccc5n6-c5ccccc5)cc4)c4ccccc4c3o2)cc1]

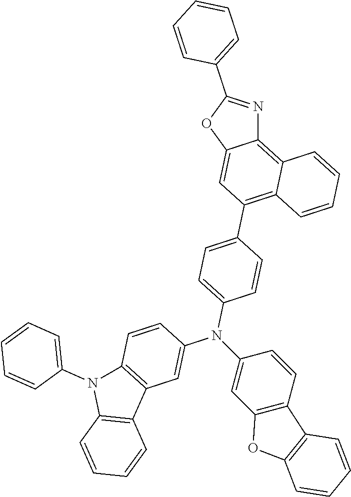 [SMILES: c1ccc(-c2nc3c(cc(-c4ccc(N(c5ccc6c(c5)oc5ccccc56)c5ccc6c(c5)c5ccccc5n6-c5ccccc5)cc4)c4ccccc43)o2)cc1]

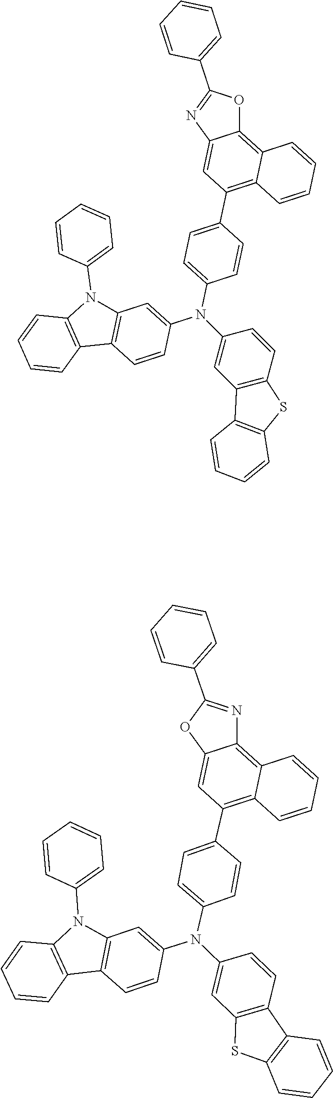 [SMILES: c1ccc(-c2nc3cc(-c4ccc(N(c5ccc6sc7ccccc7c6c5)c5ccc6c7ccccc7n(-c7ccccc7)c6c5)cc4)c4ccccc4c3o2)cc1 ; c1ccc(-c2nc3c(cc(-c4ccc(N(c5ccc6c(c5)sc5ccccc56)c5ccc6c7ccccc7n(-c7ccccc7)c6c5)cc4)c4ccccc43)o2)cc1]

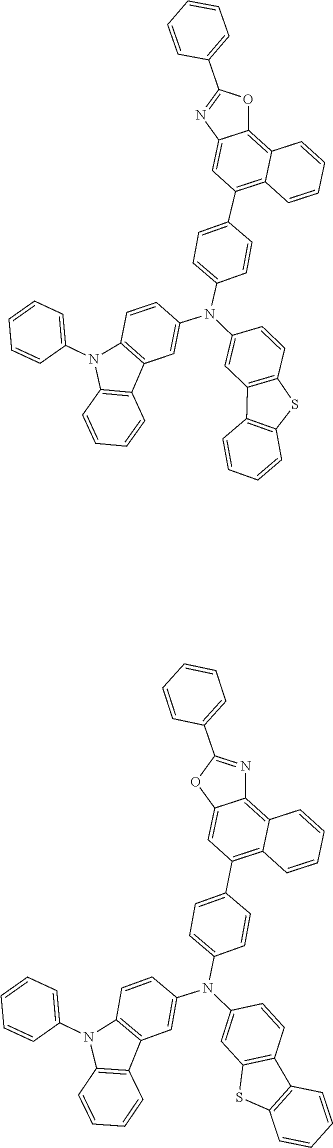 [SMILES: c1ccc(-c2nc3cc(-c4ccc(N(c5ccc6sc7ccccc7c6c5)c5ccc6c(c5)c5ccccc5n6-c5ccccc5)cc4)c4ccccc4c3o2)cc1 ; c1ccc(-c2nc3c(cc(-c4ccc(N(c5ccc6c(c5)sc5ccccc56)c5ccc6c(c5)c5ccccc5n6-c5ccccc5)cc4)c4ccccc43)o2)cc1]

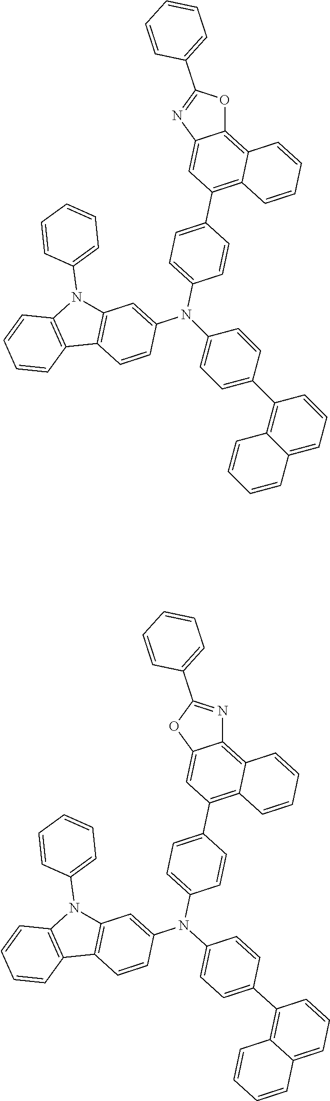 [SMILES: c1ccc(-c2nc3cc(-c4ccc(N(c5ccc(-c6cccc7ccccc67)cc5)c5ccc6c7ccccc7n(-c7ccccc7)c6c5)cc4)c4ccccc4c3o2)cc1 ; c1ccc(-c2nc3c(cc(-c4ccc(N(c5ccc(-c6cccc7ccccc67)cc5)c5ccc6c7ccccc7n(-c7ccccc7)c6c5)cc4)c4ccccc43)o2)cc1]

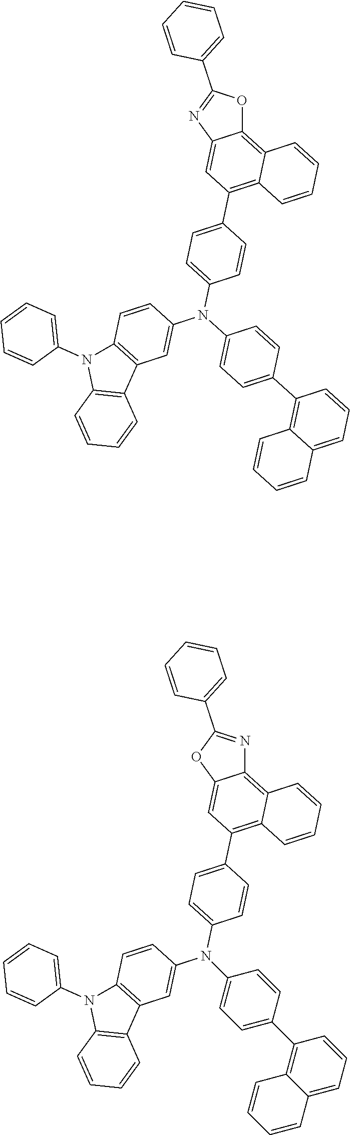 [SMILES: c1ccc(-c2nc3cc(-c4ccc(N(c5ccc(-c6cccc7ccccc67)cc5)c5ccc6c(c5)c5ccccc5n6-c5ccccc5)cc4)c4ccccc4c3o2)cc1 ; c1ccc(-c2nc3c(cc(-c4ccc(N(c5ccc(-c6cccc7ccccc67)cc5)c5ccc6c(c5)c5ccccc5n6-c5ccccc5)cc4)c4ccccc43)o2)cc1]

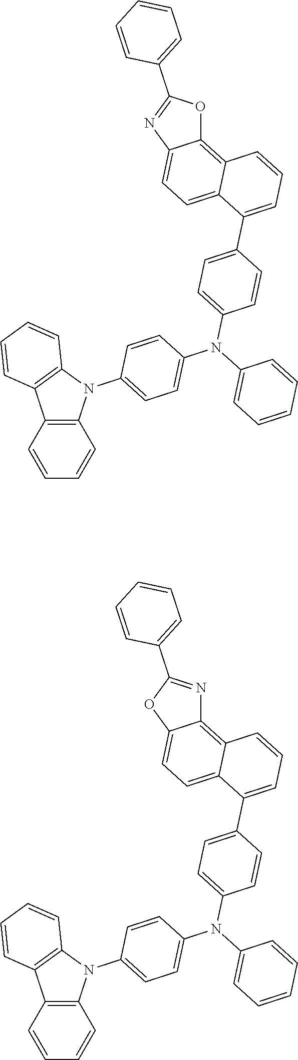 [SMILES: c1ccc(-c2nc3ccc4c(-c5ccc(N(c6ccccc6)c6ccc(-n7c8ccccc8c8ccccc87)cc6)cc5)cccc4c3o2)cc1 ; c1ccc(-c2nc3c(ccc4c(-c5ccc(N(c6ccccc6)c6ccc(-n7c8ccccc8c8ccccc87)cc6)cc5)cccc43)o2)cc1]

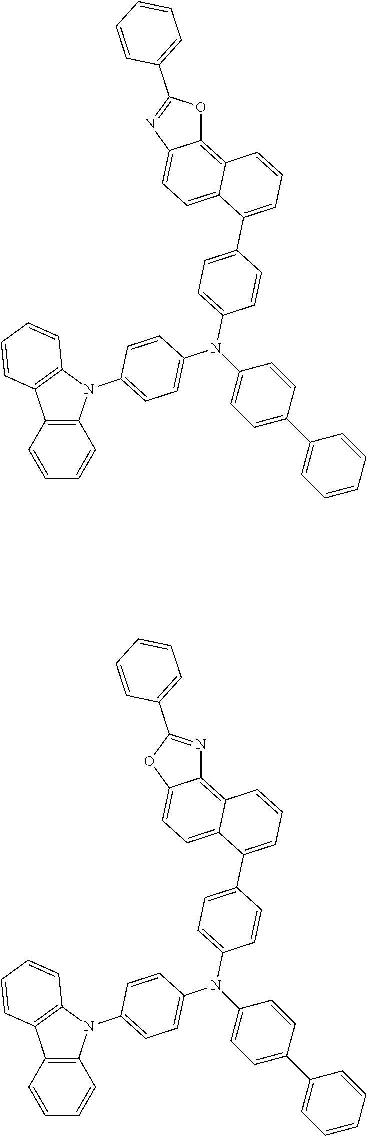 [SMILES: c1ccc(-c2ccc(N(c3ccc(-c4cccc5c4ccc4nc(-c6ccccc6)oc45)cc3)c3ccc(-n4c5ccccc5c5ccccc54)cc3)cc2)cc1 ; c1ccc(-c2ccc(N(c3ccc(-c4cccc5c4ccc4oc(-c6ccccc6)nc45)cc3)c3ccc(-n4c5ccccc5c5ccccc54)cc3)cc2)cc1]

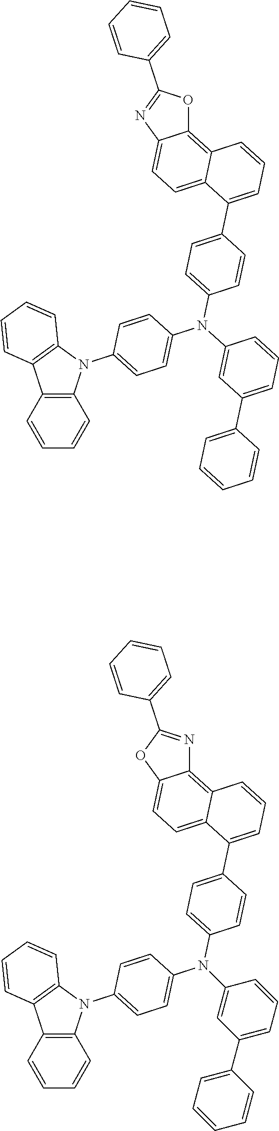 [SMILES: c1ccc(-c2cccc(N(c3ccc(-c4cccc5c4ccc4nc(-c6ccccc6)oc45)cc3)c3ccc(-n4c5ccccc5c5ccccc54)cc3)c2)cc1 ; c1ccc(-c2cccc(N(c3ccc(-c4cccc5c4ccc4oc(-c6ccccc6)nc45)cc3)c3ccc(-n4c5ccccc5c5ccccc54)cc3)c2)cc1]

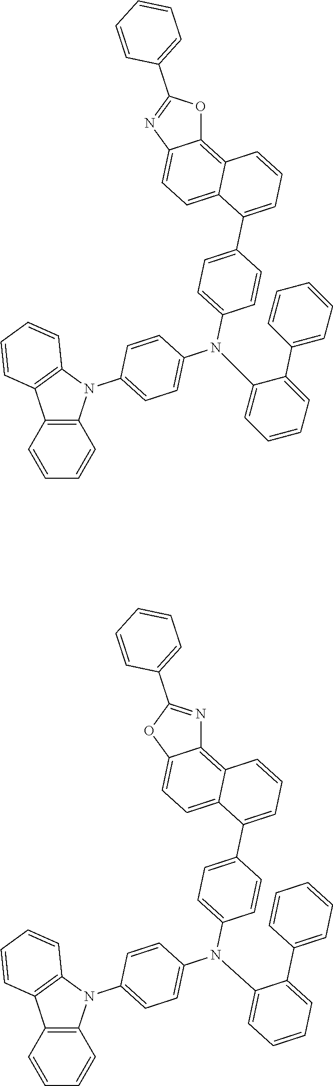 [SMILES: c1ccc(-c2nc3ccc4c(-c5ccc(N(c6ccc(-n7c8ccccc8c8ccccc87)cc6)c6ccccc6-c6ccccc6)cc5)cccc4c3o2)cc1 ; c1ccc(-c2nc3c(ccc4c(-c5ccc(N(c6ccc(-n7c8ccccc8c8ccccc87)cc6)c6ccccc6-c6ccccc6)cc5)cccc43)o2)cc1]

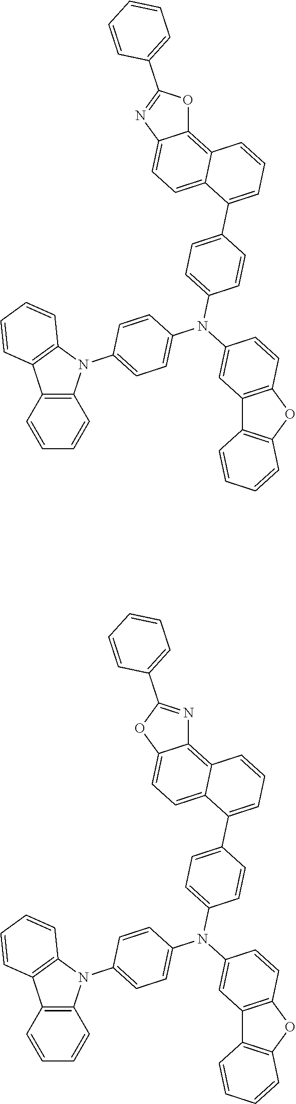 [SMILES: c1ccc(-c2nc3ccc4c(-c5ccc(N(c6ccc(-n7c8ccccc8c8ccccc87)cc6)c6ccc7oc8ccccc8c7c6)cc5)cccc4c3o2)cc1 ; c1ccc(-c2nc3c(ccc4c(-c5ccc(N(c6ccc(-n7c8ccccc8c8ccccc87)cc6)c6ccc7oc8ccccc8c7c6)cc5)cccc43)o2)cc1]

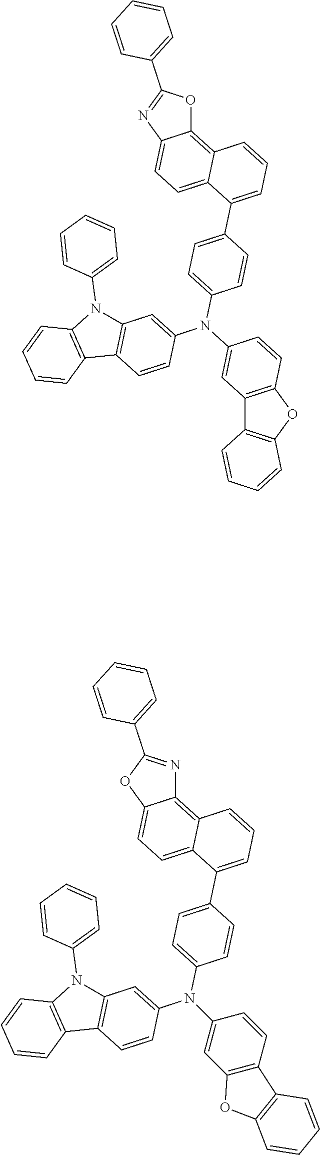 [SMILES: c1ccc(-c2nc3ccc4c(-c5ccc(N(c6ccc7oc8ccccc8c7c6)c6ccc7c8ccccc8n(-c8ccccc8)c7c6)cc5)cccc4c3o2)cc1 ; c1ccc(-c2nc3c(ccc4c(-c5ccc(N(c6ccc7c(c6)oc6ccccc67)c6ccc7c8ccccc8n(-c8ccccc8)c7c6)cc5)cccc43)o2)cc1]

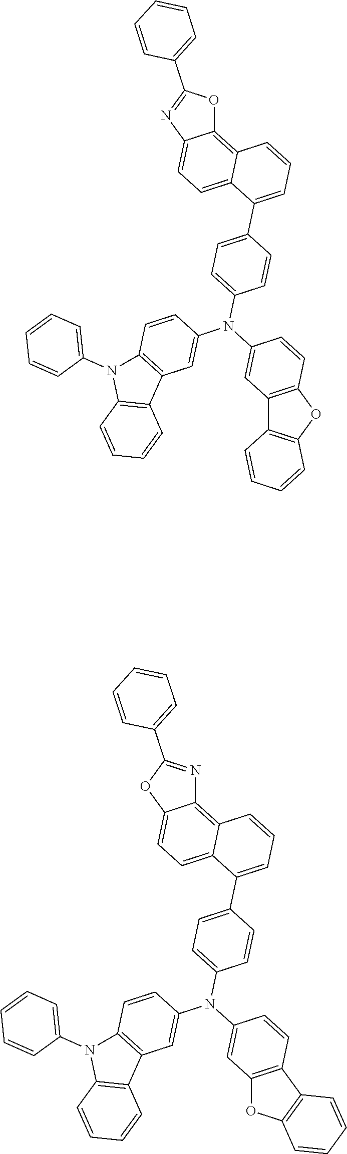 [SMILES: c1ccc(-c2nc3ccc4c(-c5ccc(N(c6ccc7oc8ccccc8c7c6)c6ccc7c(c6)c6ccccc6n7-c6ccccc6)cc5)cccc4c3o2)cc1 ; c1ccc(-c2nc3c(ccc4c(-c5ccc(N(c6ccc7c(c6)oc6ccccc67)c6ccc7c(c6)c6ccccc6n7-c6ccccc6)cc5)cccc43)o2)cc1]

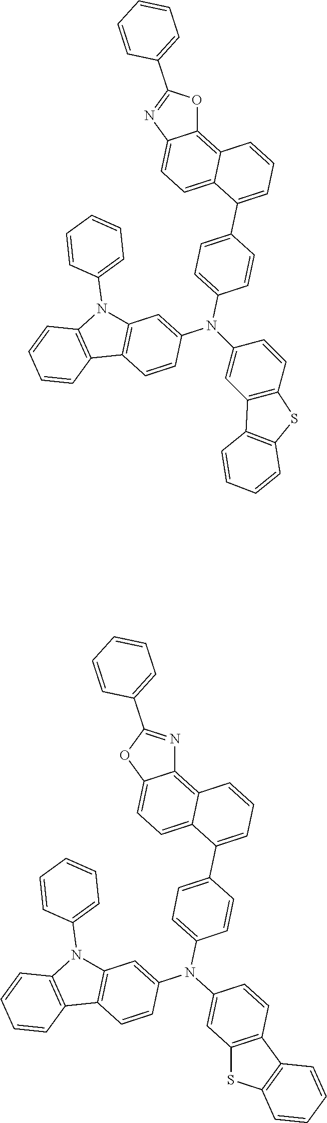 [SMILES: c1ccc(-c2nc3ccc4c(-c5ccc(N(c6ccc7sc8ccccc8c7c6)c6ccc7c8ccccc8n(-c8ccccc8)c7c6)cc5)cccc4c3o2)cc1 ; c1ccc(-c2nc3c(ccc4c(-c5ccc(N(c6ccc7c(c6)sc6ccccc67)c6ccc7c8ccccc8n(-c8ccccc8)c7c6)cc5)cccc43)o2)cc1]

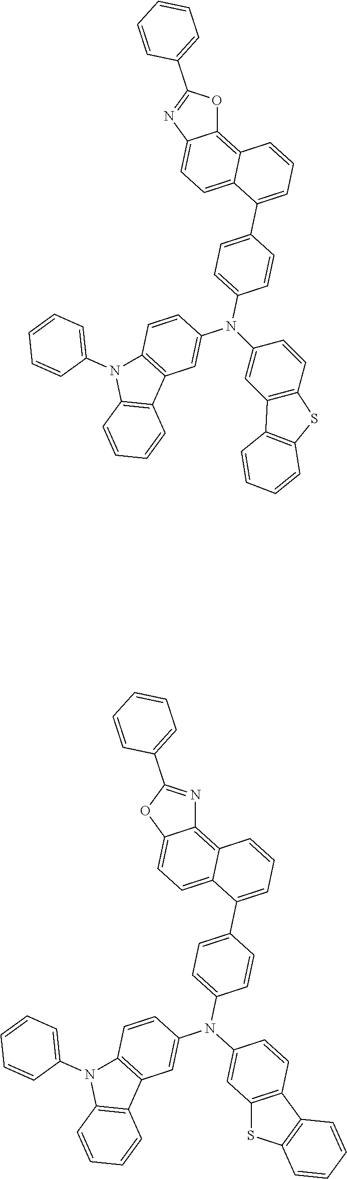 [SMILES: c1ccc(-c2nc3ccc4c(-c5ccc(N(c6ccc7sc8ccccc8c7c6)c6ccc7c(c6)c6ccccc6n7-c6ccccc6)cc5)cccc4c3o2)cc1 ; c1ccc(-c2nc3c(ccc4c(-c5ccc(N(c6ccc7c(c6)sc6ccccc67)c6ccc7c(c6)c6ccccc6n7-c6ccccc6)cc5)cccc43)o2)cc1]

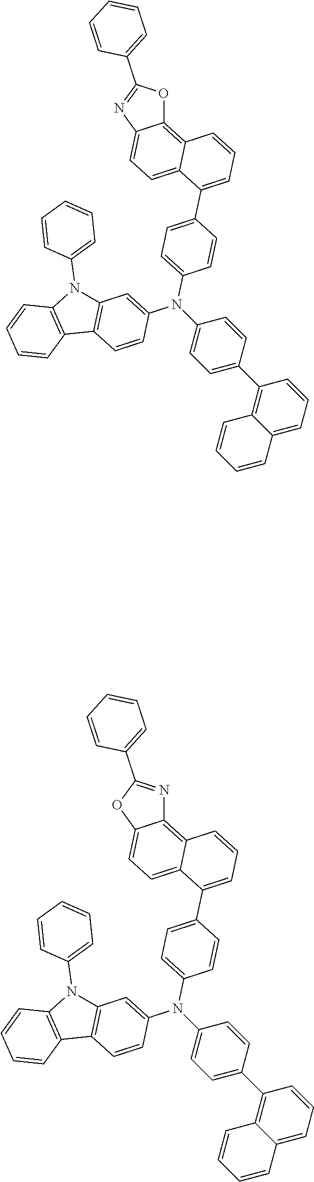 [SMILES: c1ccc(-c2nc3ccc4c(-c5ccc(N(c6ccc(-c7cccc8ccccc78)cc6)c6ccc7c8ccccc8n(-c8ccccc8)c7c6)cc5)cccc4c3o2)cc1 ; c1ccc(-c2nc3c(ccc4c(-c5ccc(N(c6ccc(-c7cccc8ccccc78)cc6)c6ccc7c8ccccc8n(-c8ccccc8)c7c6)cc5)cccc43)o2)cc1]

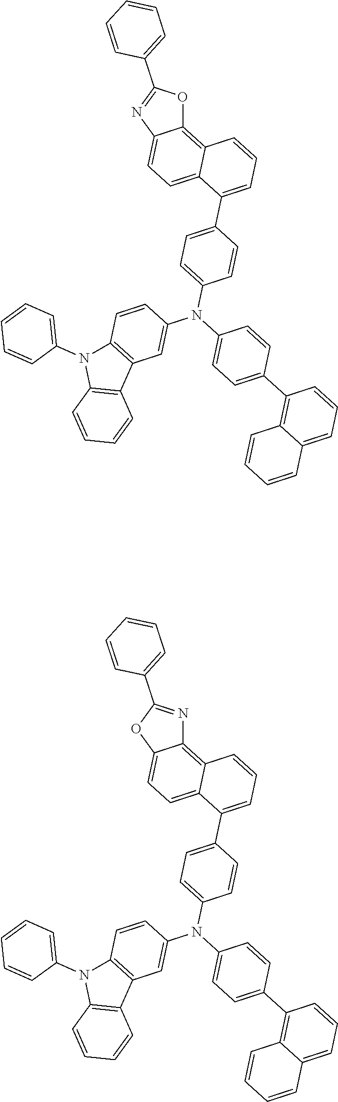 [SMILES: c1ccc(-c2nc3ccc4c(-c5ccc(N(c6ccc(-c7cccc8ccccc78)cc6)c6ccc7c(c6)c6ccccc6n7-c6ccccc6)cc5)cccc4c3o2)cc1 ; c1ccc(-c2nc3c(ccc4c(-c5ccc(N(c6ccc(-c7cccc8ccccc78)cc6)c6ccc7c(c6)c6ccccc6n7-c6ccccc6)cc5)cccc43)o2)cc1]

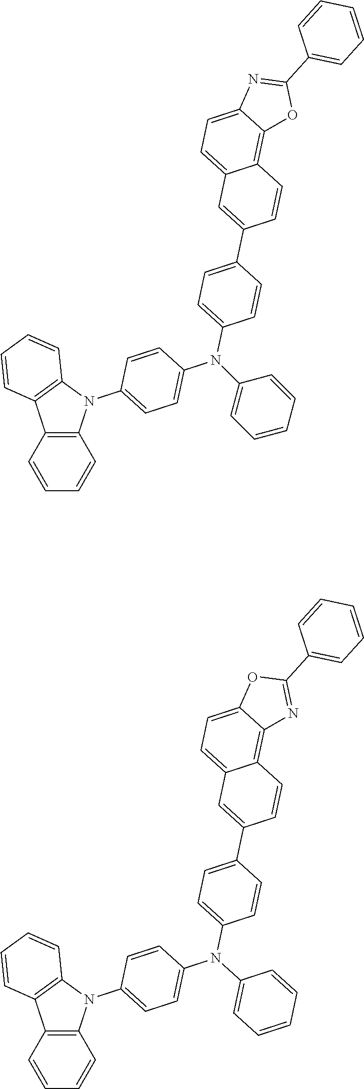 [SMILES: c1ccc(-c2nc3ccc4cc(-c5ccc(N(c6ccccc6)c6ccc(-n7c8ccccc8c8ccccc87)cc6)cc5)ccc4c3o2)cc1 ; c1ccc(-c2nc3c(ccc4cc(-c5ccc(N(c6ccccc6)c6ccc(-n7c8ccccc8c8ccccc87)cc6)cc5)ccc43)o2)cc1]

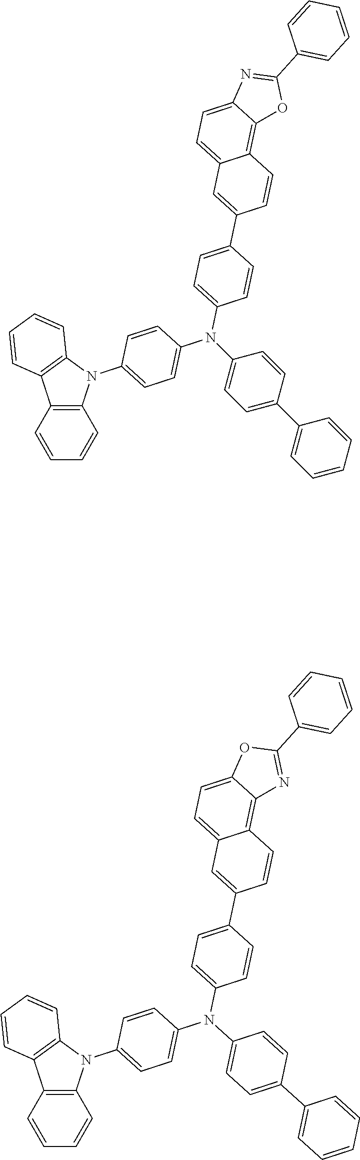 [SMILES: c1ccc(-c2ccc(N(c3ccc(-c4ccc5c(ccc6nc(-c7ccccc7)oc65)c4)cc3)c3ccc(-n4c5ccccc5c5ccccc54)cc3)cc2)cc1 ; c1ccc(-c2ccc(N(c3ccc(-c4ccc5c(ccc6oc(-c7ccccc7)nc65)c4)cc3)c3ccc(-n4c5ccccc5c5ccccc54)cc3)cc2)cc1]

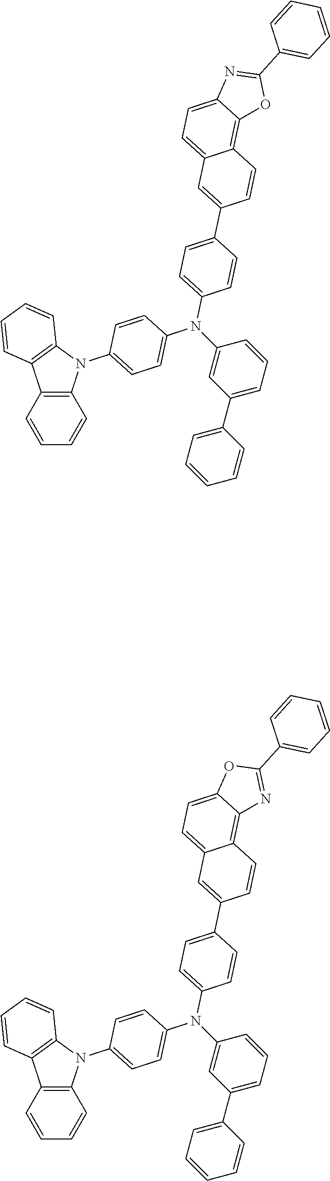 [SMILES: c1ccc(-c2cccc(N(c3ccc(-c4ccc5c(ccc6nc(-c7ccccc7)oc65)c4)cc3)c3ccc(-n4c5ccccc5c5ccccc54)cc3)c2)cc1 ; c1ccc(-c2cccc(N(c3ccc(-c4ccc5c(ccc6oc(-c7ccccc7)nc65)c4)cc3)c3ccc(-n4c5ccccc5c5ccccc54)cc3)c2)cc1]

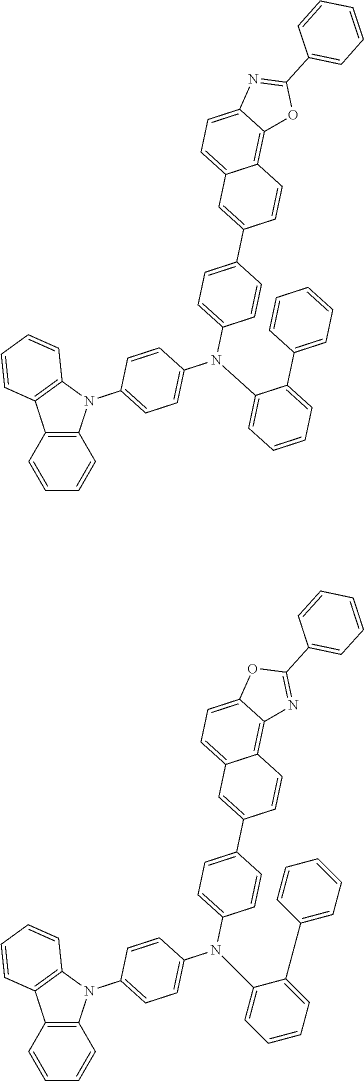 [SMILES: c1ccc(-c2nc3ccc4cc(-c5ccc(N(c6ccc(-n7c8ccccc8c8ccccc87)cc6)c6ccccc6-c6ccccc6)cc5)ccc4c3o2)cc1 ; c1ccc(-c2nc3c(ccc4cc(-c5ccc(N(c6ccc(-n7c8ccccc8c8ccccc87)cc6)c6ccccc6-c6ccccc6)cc5)ccc43)o2)cc1]

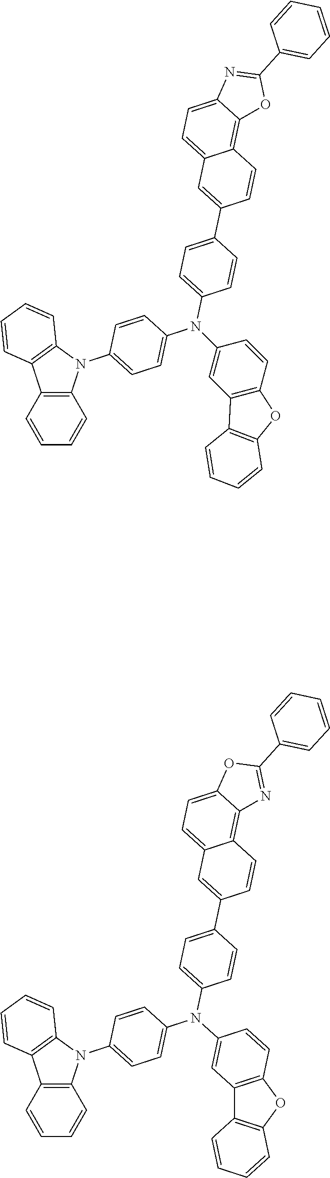 [SMILES: c1ccc(-c2nc3ccc4cc(-c5ccc(N(c6ccc(-n7c8ccccc8c8ccccc87)cc6)c6ccc7oc8ccccc8c7c6)cc5)ccc4c3o2)cc1 ; c1ccc(-c2nc3c(ccc4cc(-c5ccc(N(c6ccc(-n7c8ccccc8c8ccccc87)cc6)c6ccc7oc8ccccc8c7c6)cc5)ccc43)o2)cc1]

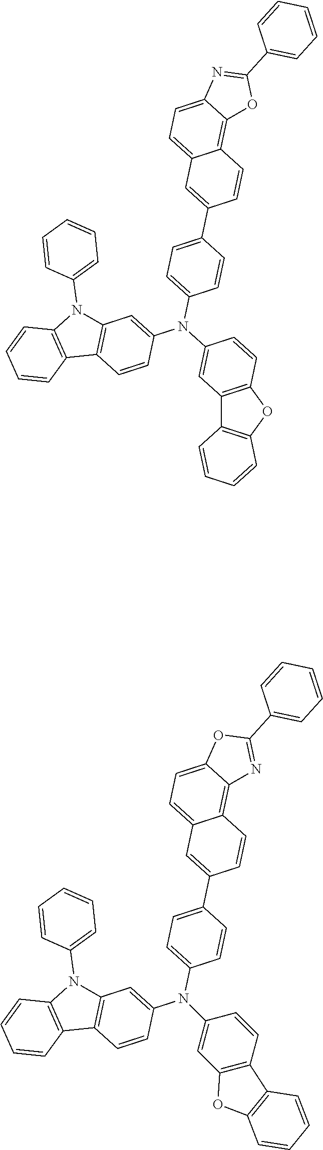 [SMILES: c1ccc(-c2nc3ccc4cc(-c5ccc(N(c6ccc7oc8ccccc8c7c6)c6ccc7c8ccccc8n(-c8ccccc8)c7c6)cc5)ccc4c3o2)cc1 ; c1ccc(-c2nc3c(ccc4cc(-c5ccc(N(c6ccc7c(c6)oc6ccccc67)c6ccc7c8ccccc8n(-c8ccccc8)c7c6)cc5)ccc43)o2)cc1]

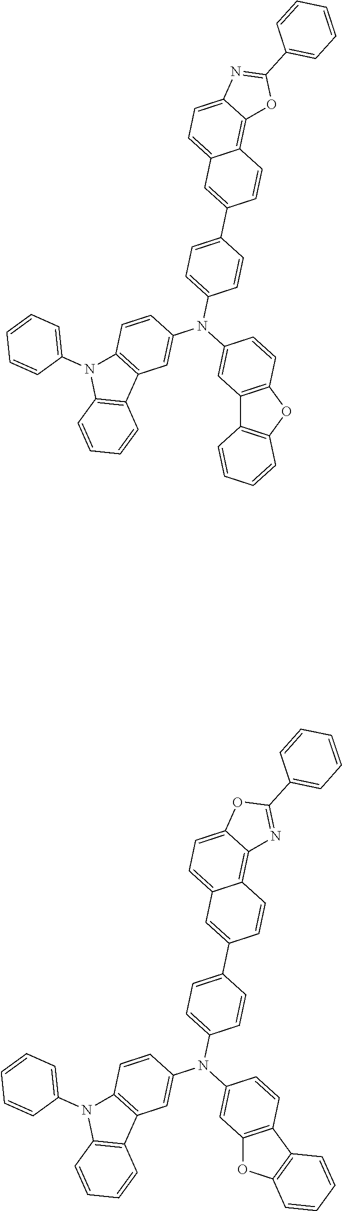 [SMILES: c1ccc(-c2nc3ccc4cc(-c5ccc(N(c6ccc7oc8ccccc8c7c6)c6ccc7c(c6)c6ccccc6n7-c6ccccc6)cc5)ccc4c3o2)cc1 ; c1ccc(-c2nc3c(ccc4cc(-c5ccc(N(c6ccc7c(c6)oc6ccccc67)c6ccc7c(c6)c6ccccc6n7-c6ccccc6)cc5)ccc43)o2)cc1]

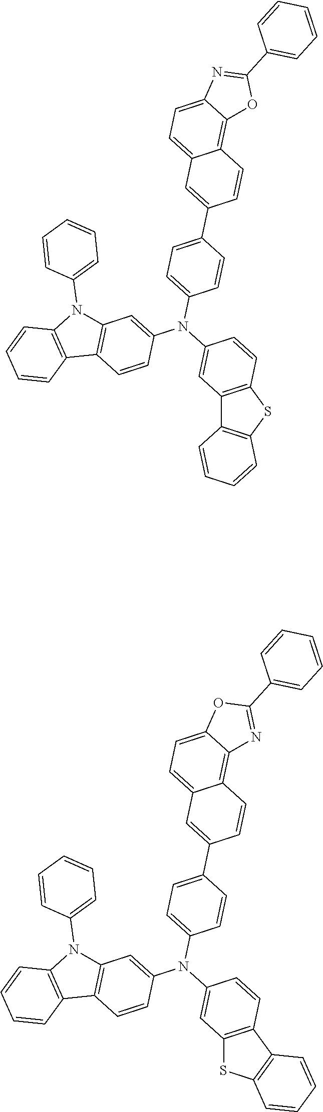 [SMILES: c1ccc(-c2nc3ccc4cc(-c5ccc(N(c6ccc7sc8ccccc8c7c6)c6ccc7c8ccccc8n(-c8ccccc8)c7c6)cc5)ccc4c3o2)cc1 ; c1ccc(-c2nc3c(ccc4cc(-c5ccc(N(c6ccc7c(c6)sc6ccccc67)c6ccc7c8ccccc8n(-c8ccccc8)c7c6)cc5)ccc43)o2)cc1]

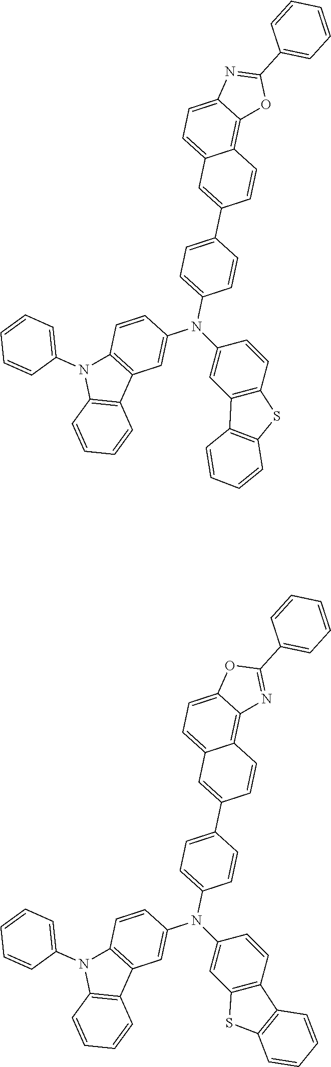 [SMILES: c1ccc(-c2nc3ccc4cc(-c5ccc(N(c6ccc7sc8ccccc8c7c6)c6ccc7c(c6)c6ccccc6n7-c6ccccc6)cc5)ccc4c3o2)cc1 ; c1ccc(-c2nc3c(ccc4cc(-c5ccc(N(c6ccc7c(c6)sc6ccccc67)c6ccc7c(c6)c6ccccc6n7-c6ccccc6)cc5)ccc43)o2)cc1]

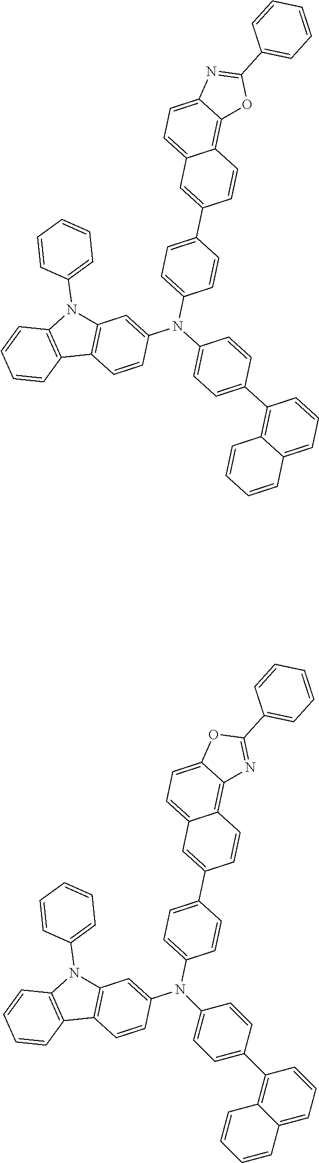 [SMILES: c1ccc(-c2nc3ccc4cc(-c5ccc(N(c6ccc(-c7cccc8ccccc78)cc6)c6ccc7c8ccccc8n(-c8ccccc8)c7c6)cc5)ccc4c3o2)cc1 ; c1ccc(-c2nc3c(ccc4cc(-c5ccc(N(c6ccc(-c7cccc8ccccc78)cc6)c6ccc7c8ccccc8n(-c8ccccc8)c7c6)cc5)ccc43)o2)cc1]

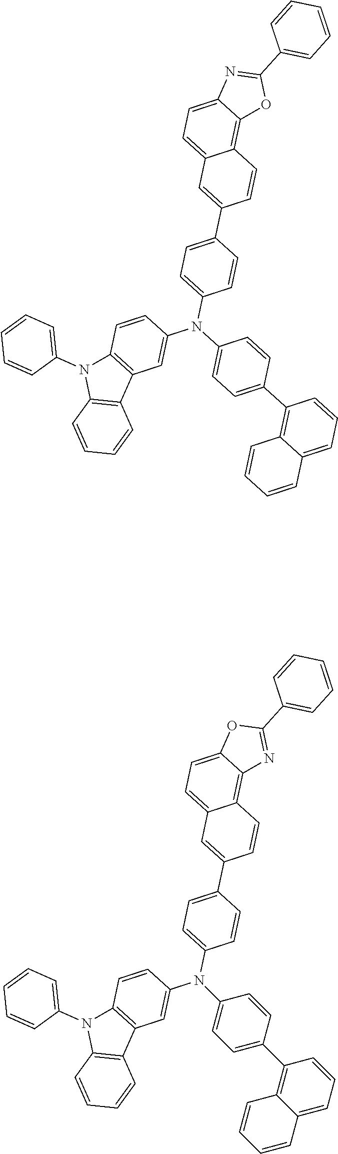 [SMILES: c1ccc(-c2nc3ccc4cc(-c5ccc(N(c6ccc(-c7cccc8ccccc78)cc6)c6ccc7c(c6)c6ccccc6n7-c6ccccc6)cc5)ccc4c3o2)cc1 ; c1ccc(-c2nc3c(ccc4cc(-c5ccc(N(c6ccc(-c7cccc8ccccc78)cc6)c6ccc7c(c6)c6ccccc6n7-c6ccccc6)cc5)ccc43)o2)cc1]

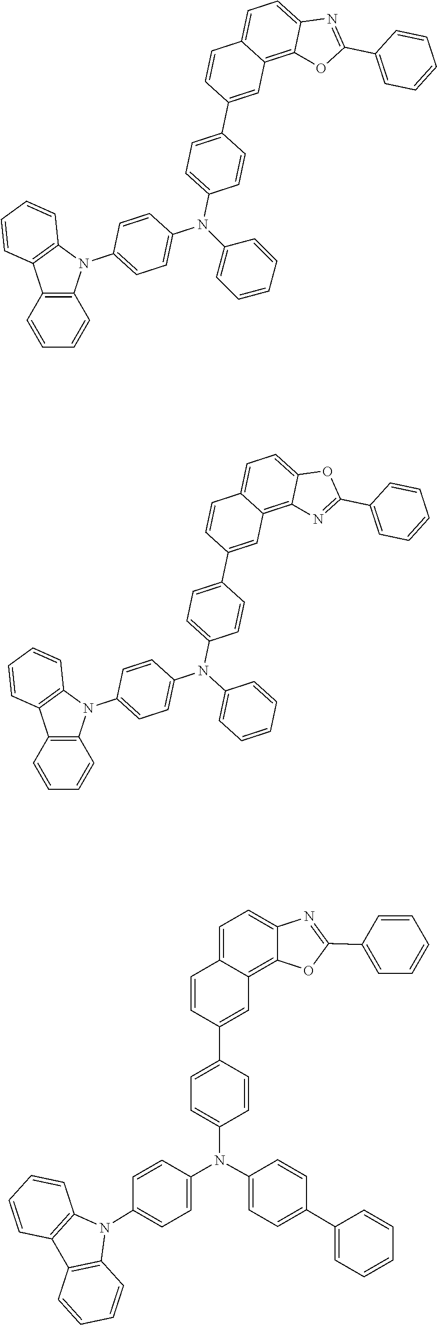 [SMILES: c1ccc(-c2nc3ccc4ccc(-c5ccc(N(c6ccccc6)c6ccc(-n7c8ccccc8c8ccccc87)cc6)cc5)cc4c3o2)cc1 ; c1ccc(-c2nc3c(ccc4ccc(-c5ccc(N(c6ccccc6)c6ccc(-n7c8ccccc8c8ccccc87)cc6)cc5)cc43)o2)cc1 ; c1ccc(-c2ccc(N(c3ccc(-c4ccc5ccc6nc(-c7ccccc7)oc6c5c4)cc3)c3ccc(-n4c5ccccc5c5ccccc54)cc3)cc2)cc1]

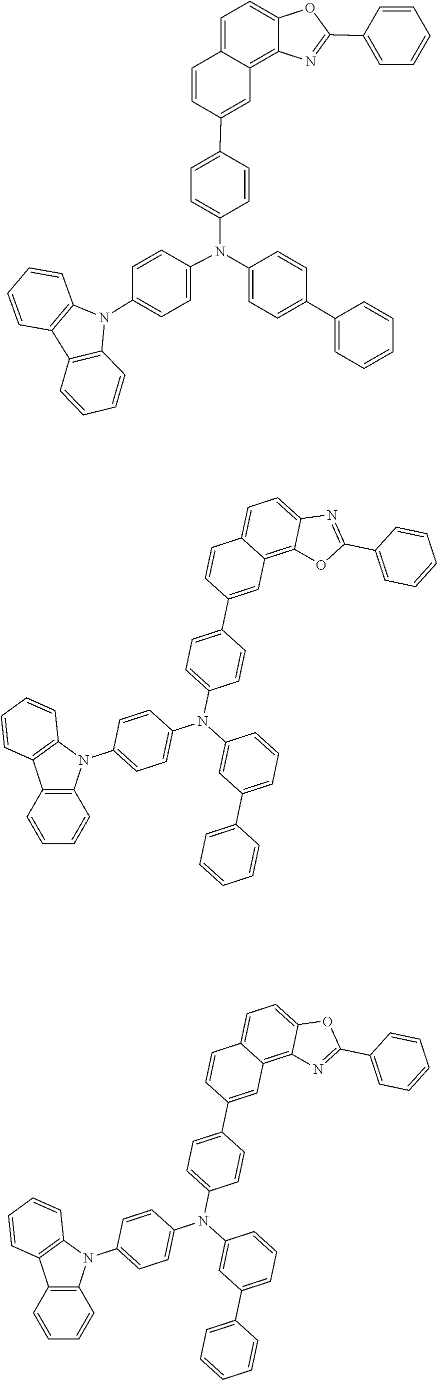 [SMILES: c1ccc(-c2ccc(N(c3ccc(-c4ccc5ccc6oc(-c7ccccc7)nc6c5c4)cc3)c3ccc(-n4c5ccccc5c5ccccc54)cc3)cc2)cc1 ; c1ccc(-c2cccc(N(c3ccc(-c4ccc5ccc6nc(-c7ccccc7)oc6c5c4)cc3)c3ccc(-n4c5ccccc5c5ccccc54)cc3)c2)cc1 ; c1ccc(-c2cccc(N(c3ccc(-c4ccc5ccc6oc(-c7ccccc7)nc6c5c4)cc3)c3ccc(-n4c5ccccc5c5ccccc54)cc3)c2)cc1]

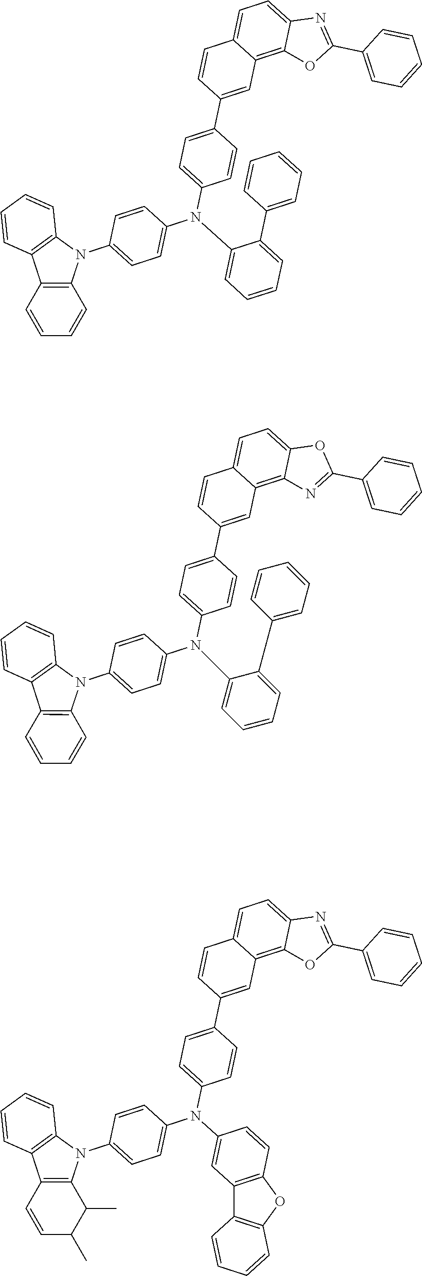 [SMILES: c1ccc(-c2nc3ccc4ccc(-c5ccc(N(c6ccc(-n7c8ccccc8c8ccccc87)cc6)c6ccccc6-c6ccccc6)cc5)cc4c3o2)cc1 ; c1ccc(-c2nc3c(ccc4ccc(-c5ccc(N(c6ccc(-n7c8ccccc8c8ccccc87)cc6)c6ccccc6-c6ccccc6)cc5)cc43)o2)cc1 ; CC1C=Cc2c(n(-c3ccc(N(c4ccc(-c5ccc6ccc7nc(-c8ccccc8)oc7c6c5)cc4)c4ccc5oc6ccccc6c5c4)cc3)c3ccccc23)C1C]

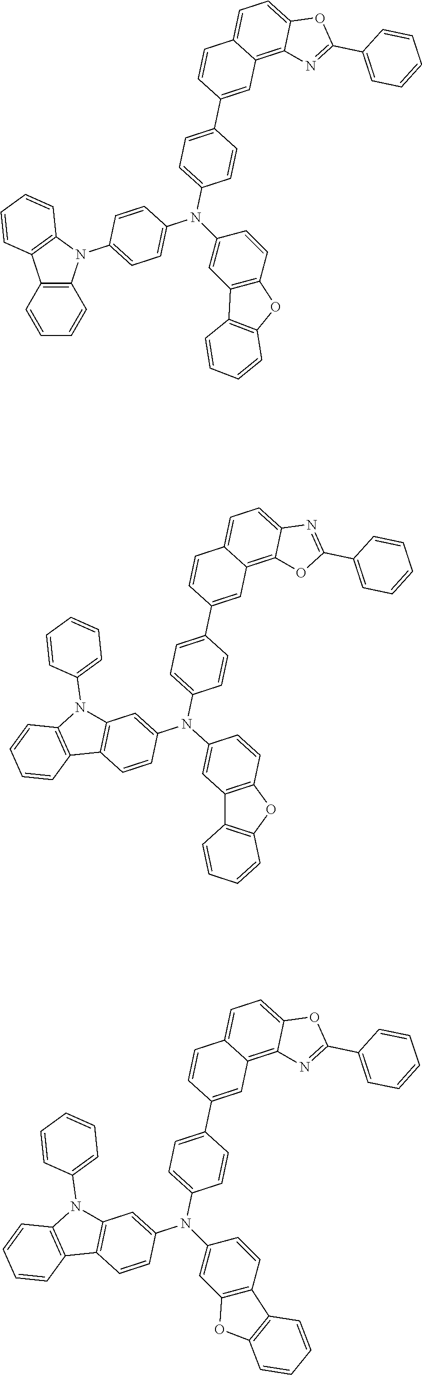 [SMILES: c1ccc(-c2nc3c(ccc4ccc(-c5ccc(N(c6ccc(-n7c8ccccc8c8ccccc87)cc6)c6ccc7oc8ccccc8c7c6)cc5)cc43)o2)cc1 ; c1ccc(-c2nc3ccc4ccc(-c5ccc(N(c6ccc7oc8ccccc8c7c6)c6ccc7c8ccccc8n(-c8ccccc8)c7c6)cc5)cc4c3o2)cc1 ; c1ccc(-c2nc3c(ccc4ccc(-c5ccc(N(c6ccc7c(c6)oc6ccccc67)c6ccc7c8ccccc8n(-c8ccccc8)c7c6)cc5)cc43)o2)cc1]

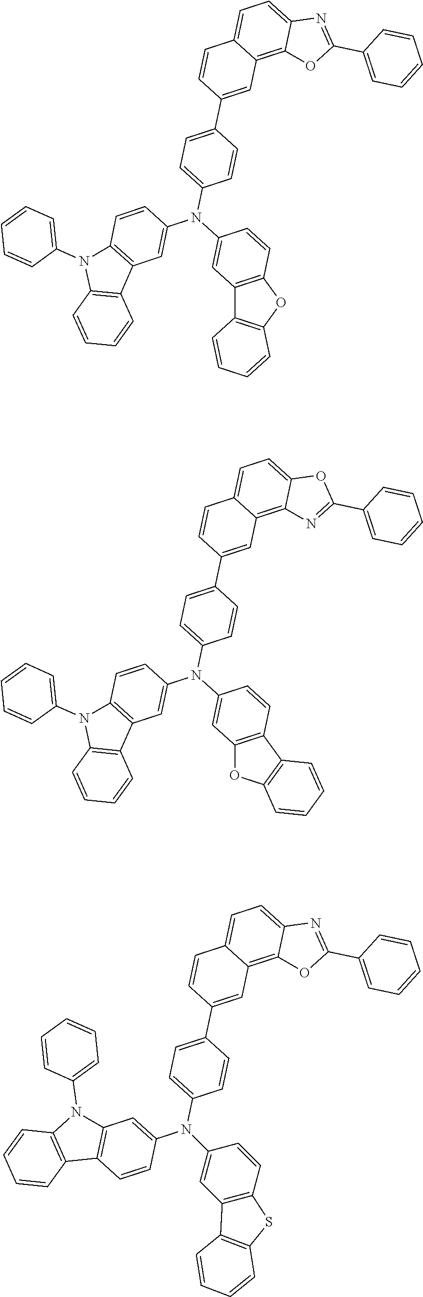 [SMILES: c1ccc(-c2nc3ccc4ccc(-c5ccc(N(c6ccc7oc8ccccc8c7c6)c6ccc7c(c6)c6ccccc6n7-c6ccccc6)cc5)cc4c3o2)cc1 ; c1ccc(-c2nc3c(ccc4ccc(-c5ccc(N(c6ccc7c(c6)oc6ccccc67)c6ccc7c(c6)c6ccccc6n7-c6ccccc6)cc5)cc43)o2)cc1 ; c1ccc(-c2nc3ccc4ccc(-c5ccc(N(c6ccc7sc8ccccc8c7c6)c6ccc7c8ccccc8n(-c8ccccc8)c7c6)cc5)cc4c3o2)cc1]

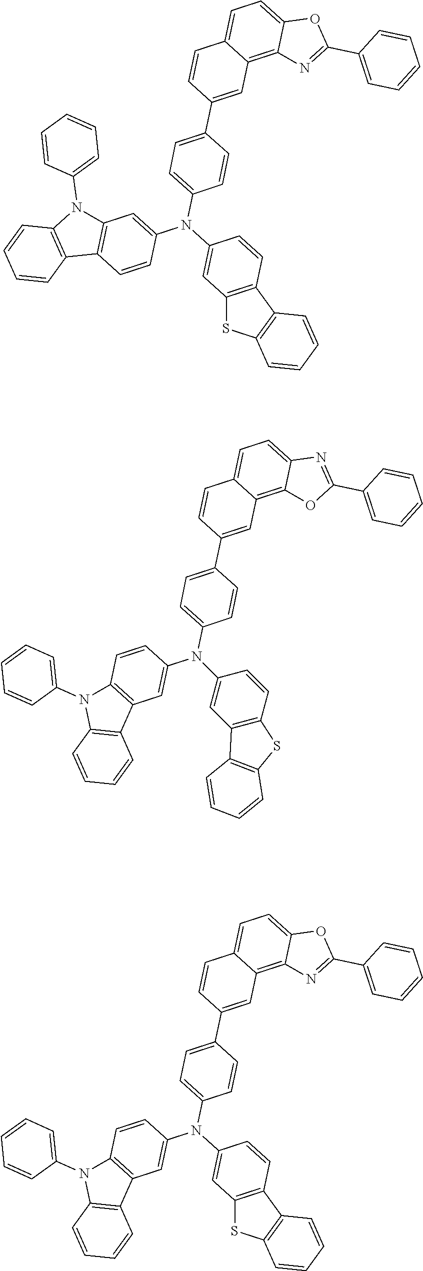 [SMILES: c1ccc(-c2nc3c(ccc4ccc(-c5ccc(N(c6ccc7c(c6)sc6ccccc67)c6ccc7c8ccccc8n(-c8ccccc8)c7c6)cc5)cc43)o2)cc1 ; c1ccc(-c2nc3ccc4ccc(-c5ccc(N(c6ccc7sc8ccccc8c7c6)c6ccc7c(c6)c6ccccc6n7-c6ccccc6)cc5)cc4c3o2)cc1 ; c1ccc(-c2nc3c(ccc4ccc(-c5ccc(N(c6ccc7c(c6)sc6ccccc67)c6ccc7c(c6)c6ccccc6n7-c6ccccc6)cc5)cc43)o2)cc1]

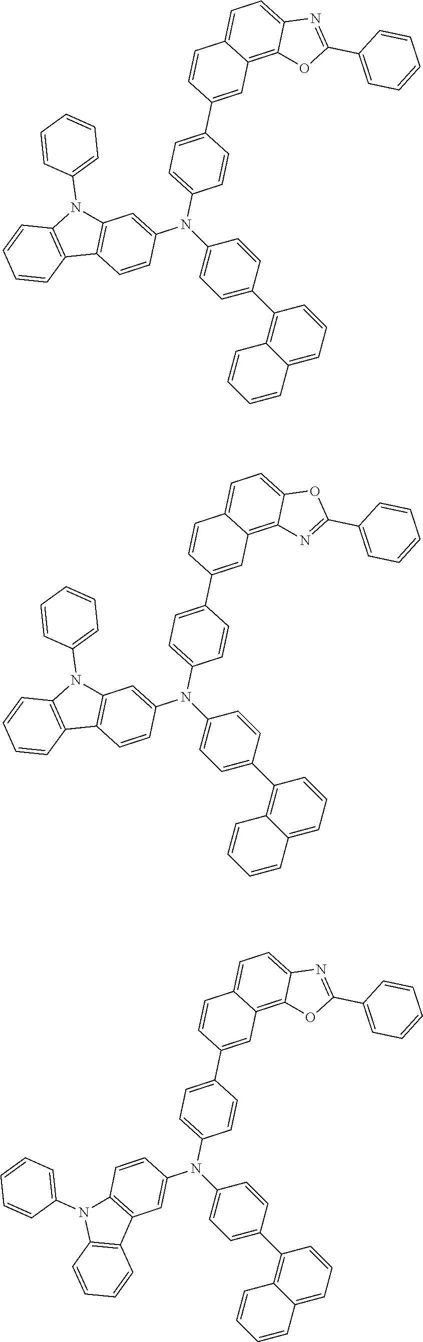 [SMILES: c1ccc(-c2nc3ccc4ccc(-c5ccc(N(c6ccc(-c7cccc8ccccc78)cc6)c6ccc7c8ccccc8n(-c8ccccc8)c7c6)cc5)cc4c3o2)cc1 ; c1ccc(-c2nc3c(ccc4ccc(-c5ccc(N(c6ccc(-c7cccc8ccccc78)cc6)c6ccc7c8ccccc8n(-c8ccccc8)c7c6)cc5)cc43)o2)cc1 ; c1ccc(-c2nc3ccc4ccc(-c5ccc(N(c6ccc(-c7cccc8ccccc78)cc6)c6ccc7c(c6)c6ccccc6n7-c6ccccc6)cc5)cc4c3o2)cc1]

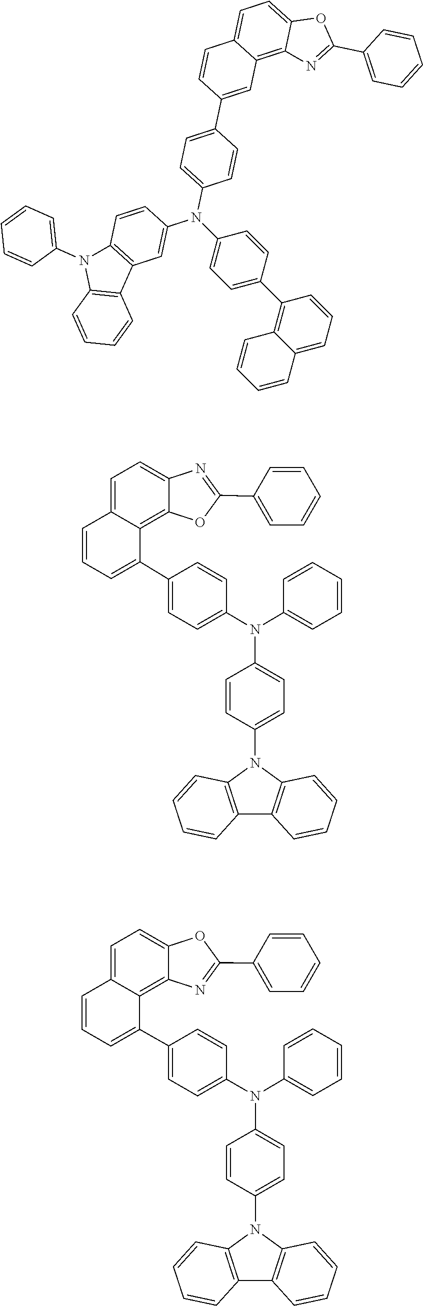 [SMILES: c1ccc(-c2nc3c(ccc4ccc(-c5ccc(N(c6ccc(-c7cccc8ccccc78)cc6)c6ccc7c(c6)c6ccccc6n7-c6ccccc6)cc5)cc43)o2)cc1 ; c1ccc(-c2nc3ccc4cccc(-c5ccc(N(c6ccccc6)c6ccc(-n7c8ccccc8c8ccccc87)cc6)cc5)c4c3o2)cc1 ; c1ccc(-c2nc3c(ccc4cccc(-c5ccc(N(c6ccccc6)c6ccc(-n7c8ccccc8c8ccccc87)cc6)cc5)c43)o2)cc1]

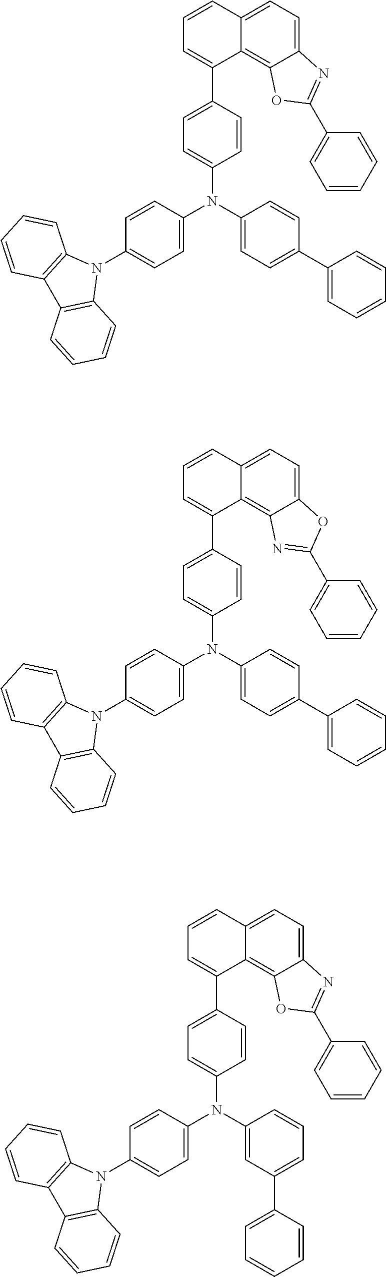 [SMILES: c1ccc(-c2ccc(N(c3ccc(-c4cccc5ccc6nc(-c7ccccc7)oc6c45)cc3)c3ccc(-n4c5ccccc5c5ccccc54)cc3)cc2)cc1 ; c1ccc(-c2ccc(N(c3ccc(-c4cccc5ccc6oc(-c7ccccc7)nc6c45)cc3)c3ccc(-n4c5ccccc5c5ccccc54)cc3)cc2)cc1 ; c1ccc(-c2cccc(N(c3ccc(-c4cccc5ccc6nc(-c7ccccc7)oc6c45)cc3)c3ccc(-n4c5ccccc5c5ccccc54)cc3)c2)cc1]

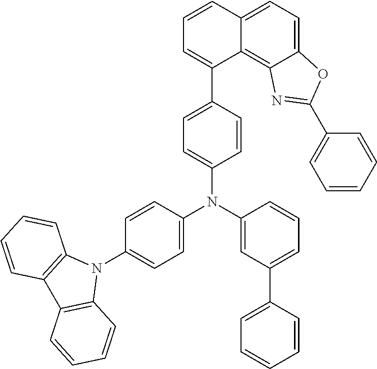 [SMILES: c1ccc(-c2cccc(N(c3ccc(-c4cccc5ccc6oc(-c7ccccc7)nc6c45)cc3)c3ccc(-n4c5ccccc5c5ccccc54)cc3)c2)cc1]

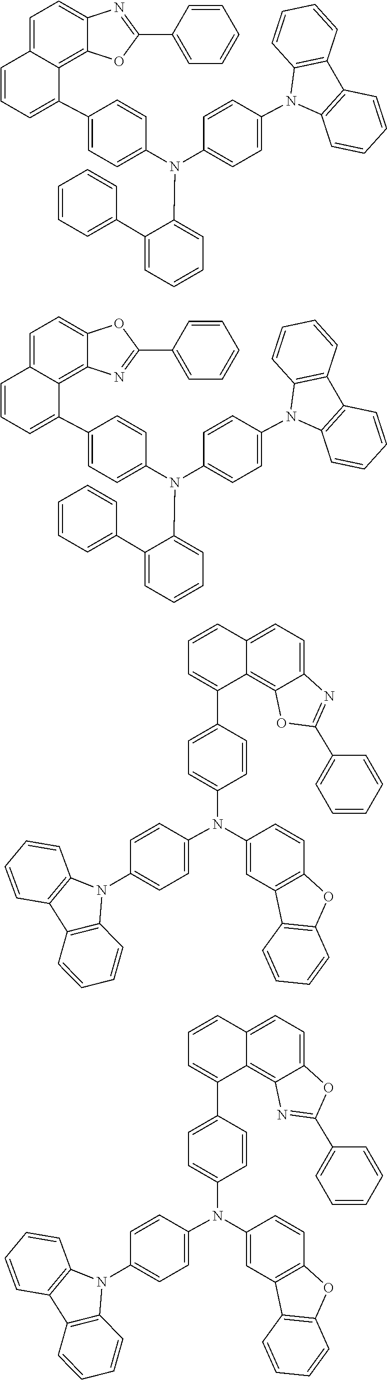

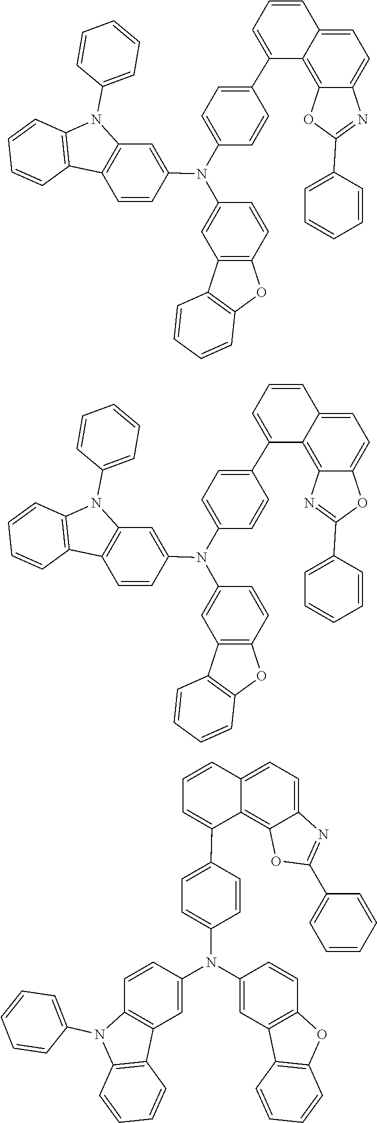

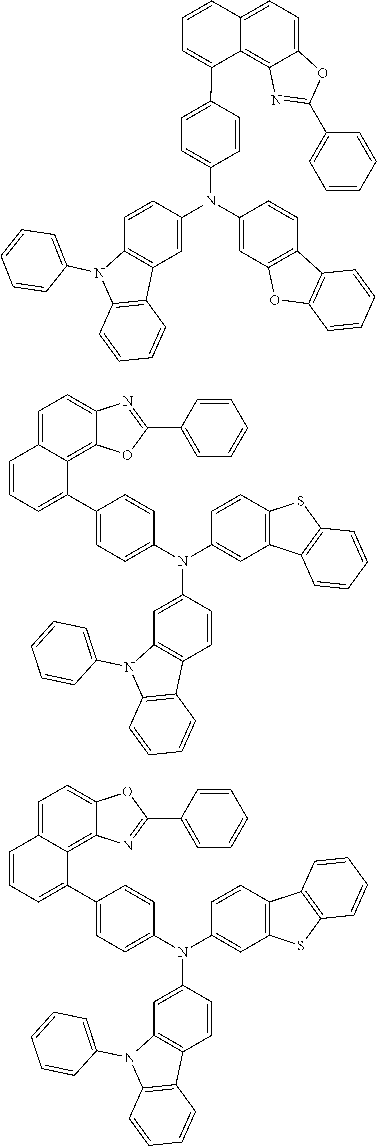

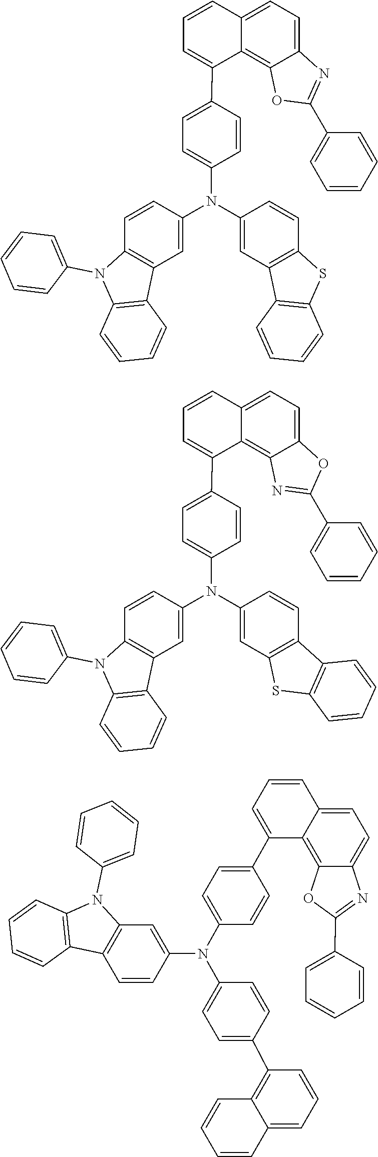

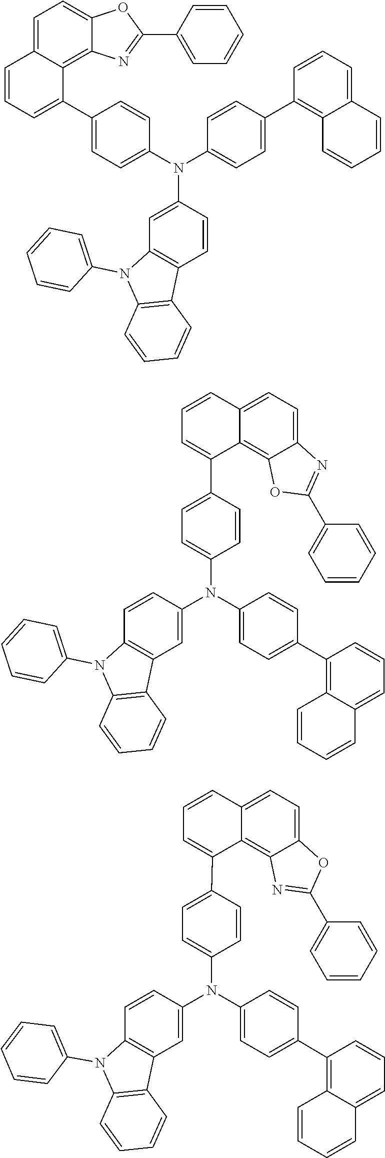 [SMILES: c1ccc(-c2nc3c(ccc4cccc(-c5ccc(N(c6ccc(-c7cccc8ccccc78)cc6)c6ccc7c8ccccc8n(-c8ccccc8)c7c6)cc5)c43)o2)cc1 ; c1ccc(-c2nc3ccc4cccc(-c5ccc(N(c6ccc(-c7cccc8ccccc78)cc6)c6ccc7c(c6)c6ccccc6n7-c6ccccc6)cc5)c4c3o2)cc1 ; c1ccc(-c2nc3c(ccc4cccc(-c5ccc(N(c6ccc(-c7cccc8ccccc78)cc6)c6ccc7c(c6)c6ccccc6n7-c6ccccc6)cc5)c43)o2)cc1]

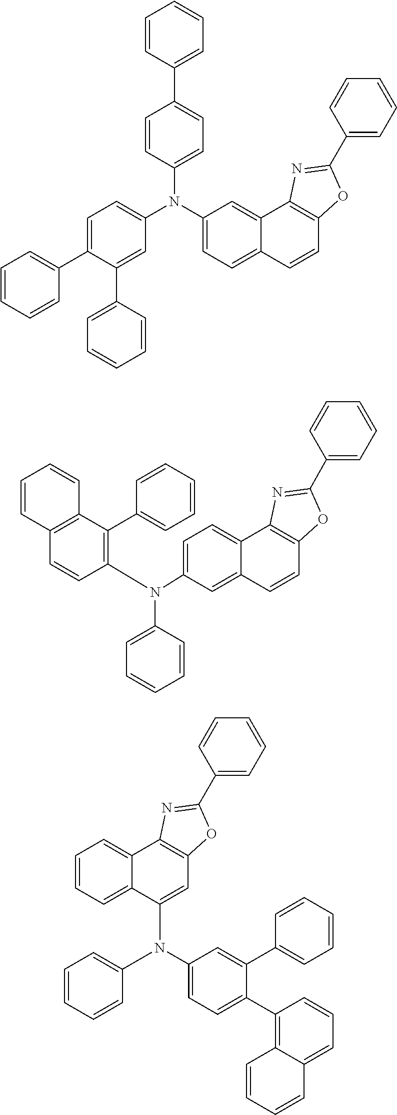 [SMILES: c1ccc(-c2ccc(N(c3ccc(-c4ccccc4)c(-c4ccccc4)c3)c3ccc4ccc5oc(-c6ccccc6)nc5c4c3)cc2)cc1 ; c1ccc(-c2nc3c(ccc4cc(N(c5ccccc5)c5ccc6ccccc6c5-c5ccccc5)ccc43)o2)cc1 ; c1ccc(-c2nc3c(cc(N(c4ccccc4)c4ccc(-c5cccc6ccccc56)c(-c5ccccc5)c4)c4ccccc43)o2)cc1]

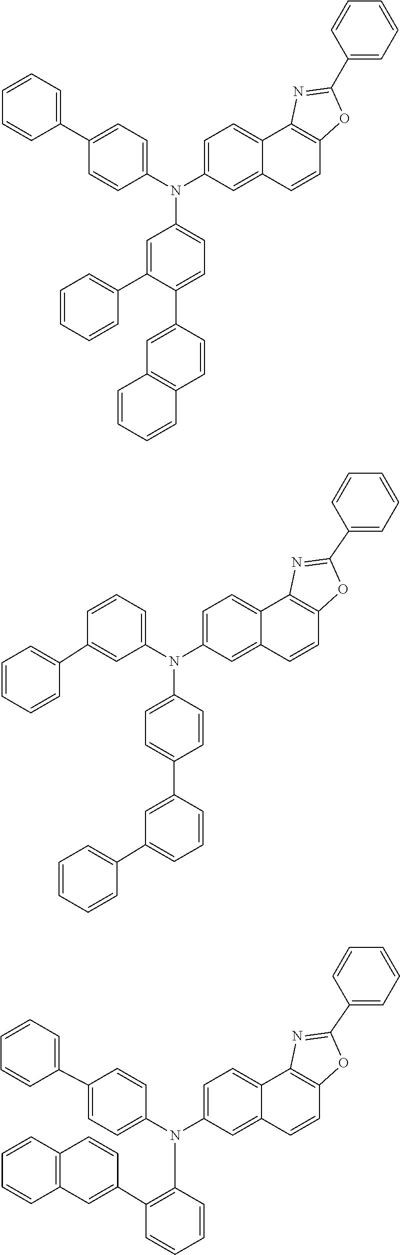 [SMILES: c1ccc(-c2ccc(N(c3ccc(-c4ccc5ccccc5c4)c(-c4ccccc4)c3)c3ccc4c(ccc5oc(-c6ccccc6)nc54)c3)cc2)cc1 ; c1ccc(-c2cccc(-c3ccc(N(c4cccc(-c5ccccc5)c4)c4ccc5c(ccc6oc(-c7ccccc7)nc65)c4)cc3)c2)cc1 ; c1ccc(-c2ccc(N(c3ccc4c(ccc5oc(-c6ccccc6)nc54)c3)c3ccccc3-c3ccc4ccccc4c3)cc2)cc1]

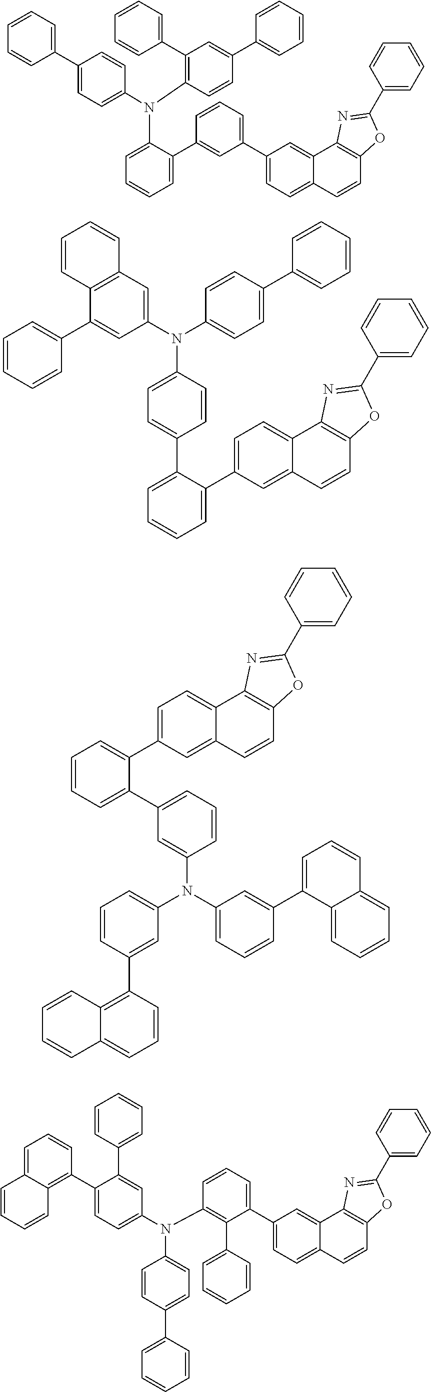

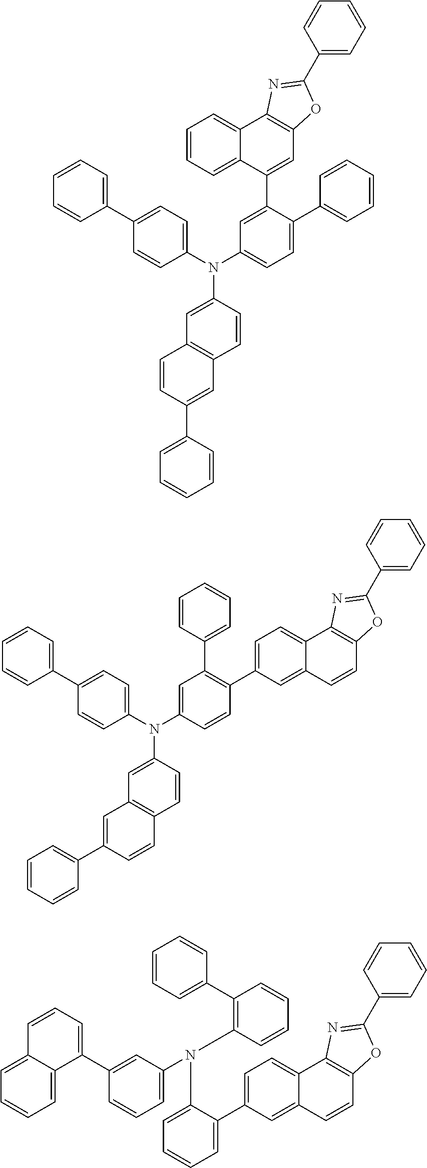 [SMILES: c1ccc(-c2ccc(N(c3ccc(-c4ccccc4)c(-c4cc5oc(-c6ccccc6)nc5c5ccccc45)c3)c3ccc4cc(-c5ccccc5)ccc4c3)cc2)cc1 ; c1ccc(-c2ccc(N(c3ccc(-c4ccc5c(ccc6oc(-c7ccccc7)nc65)c4)c(-c4ccccc4)c3)c3ccc4ccc(-c5ccccc5)cc4c3)cc2)cc1 ; c1ccc(-c2nc3c(ccc4cc(-c5ccccc5N(c5cccc(-c6cccc7ccccc67)c5)c5ccccc5-c5ccccc5)ccc43)o2)cc1]

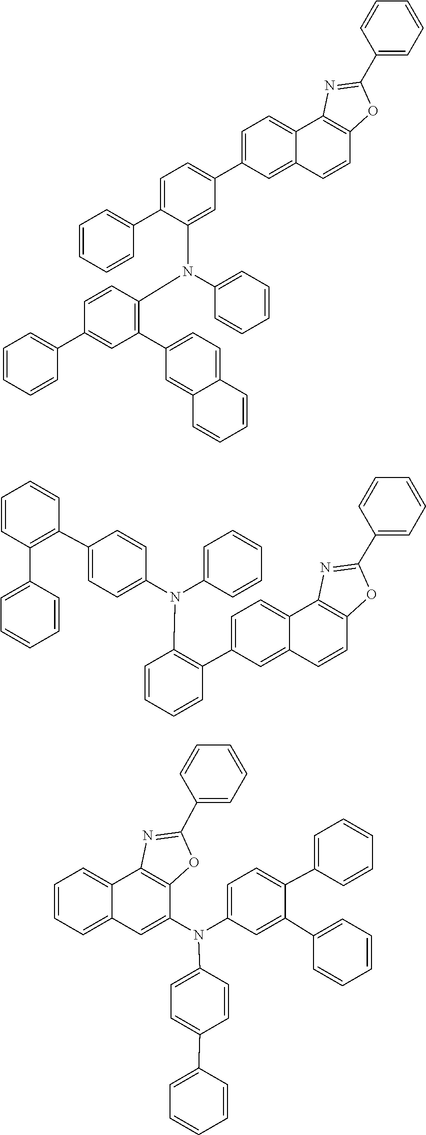

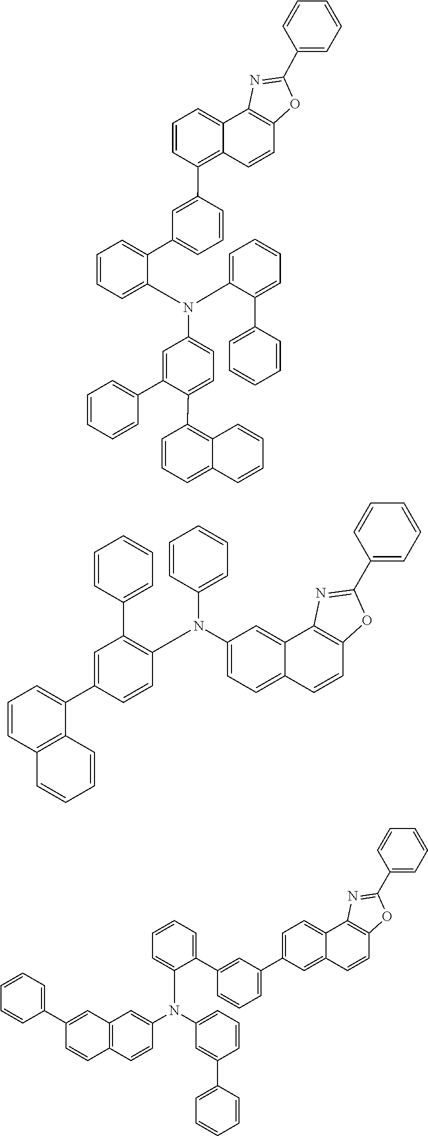 [SMILES: c1ccc(-c2nc3c(ccc4c(-c5cccc(-c6ccccc6N(c6ccc(-c7cccc8ccccc78)c(-c7ccccc7)c6)c6ccccc6-c6ccccc6)c5)cccc43)o2)cc1 ; c1ccc(-c2nc3c(ccc4ccc(N(c5ccccc5)c5ccc(-c6cccc7ccccc67)cc5-c5ccccc5)cc43)o2)cc1 ; c1ccc(-c2cccc(N(c3ccc4ccc(-c5ccccc5)cc4c3)c3ccccc3-c3cccc(-c4ccc5c(ccc6oc(-c7ccccc7)nc65)c4)c3)c2)cc1]

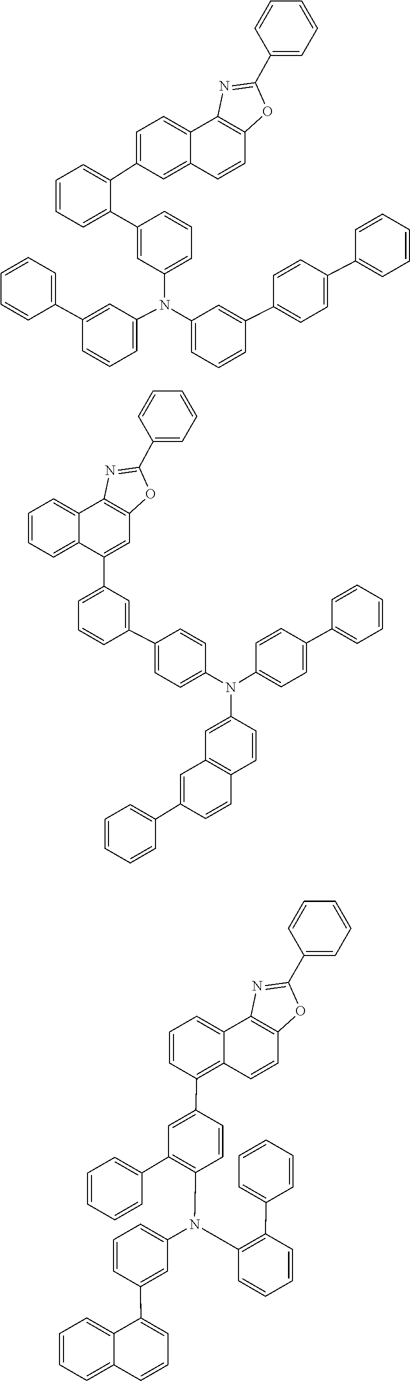

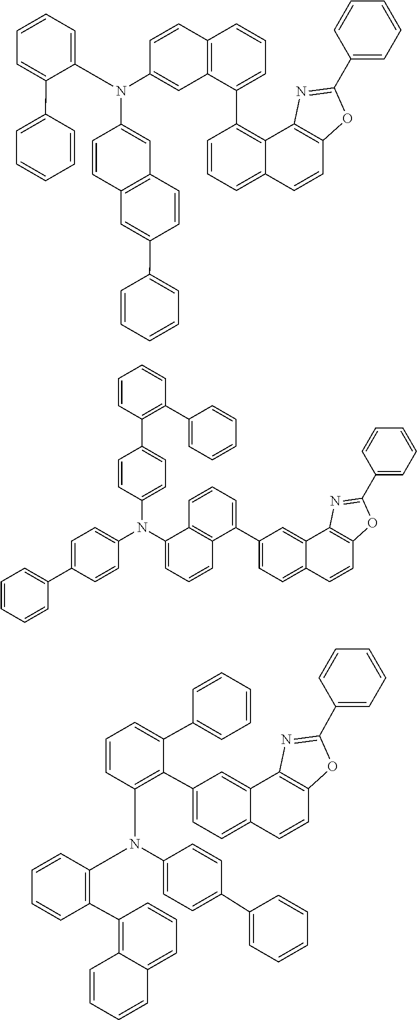

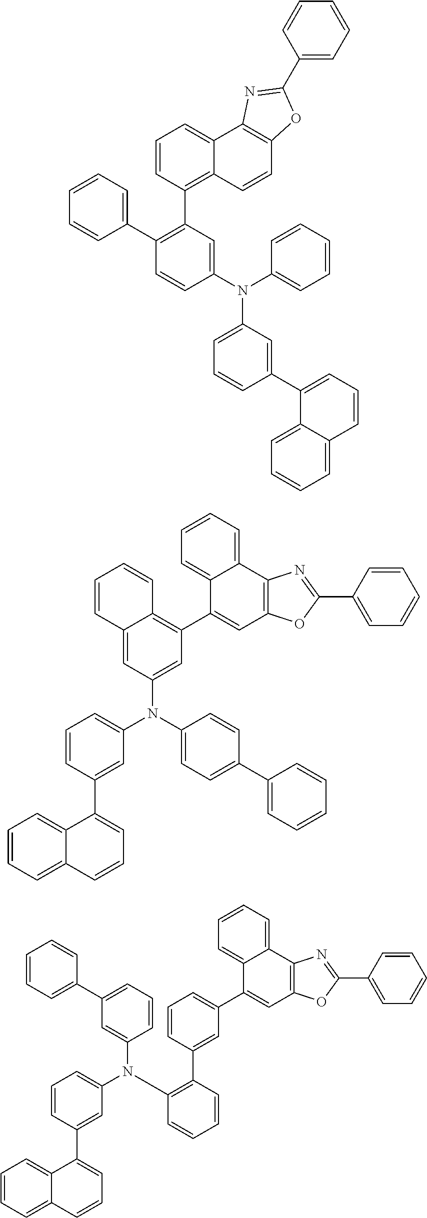 [SMILES: c1ccc(-c2nc3c(ccc4c(-c5cc(N(c6ccccc6)c6cccc(-c7cccc8ccccc78)c6)ccc5-c5ccccc5)cccc43)o2)cc1 ; c1ccc(-c2ccc(N(c3cccc(-c4cccc5ccccc45)c3)c3cc(-c4cc5oc(-c6ccccc6)nc5c5ccccc45)c4ccccc4c3)cc2)cc1 ; c1ccc(-c2cccc(N(c3cccc(-c4cccc5ccccc45)c3)c3ccccc3-c3cccc(-c4cc5oc(-c6ccccc6)nc5c5ccccc45)c3)c2)cc1]

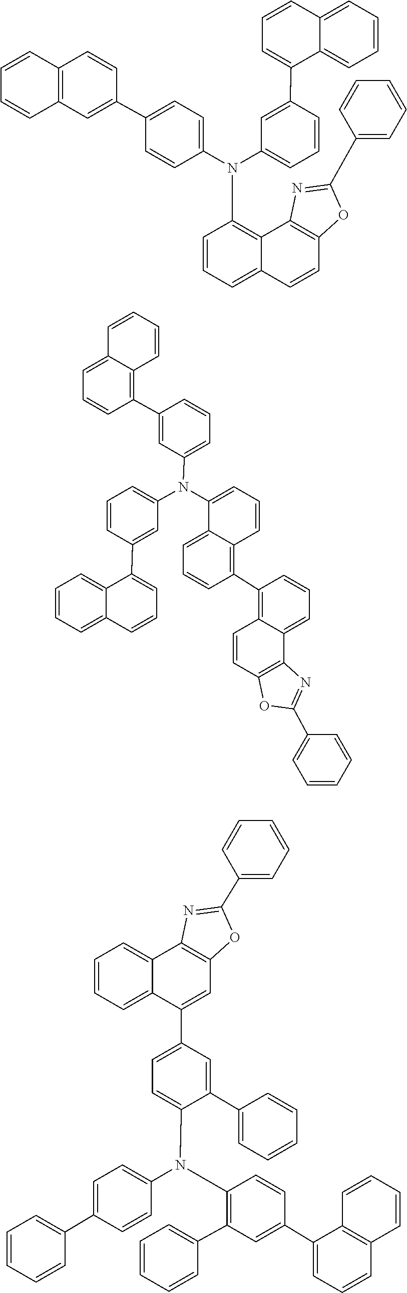 [SMILES: c1ccc(-c2nc3c(ccc4cccc(N(c5ccc(-c6ccc7ccccc7c6)cc5)c5cccc(-c6cccc7ccccc67)c5)c43)o2)cc1 ; c1ccc(-c2nc3c(ccc4c(-c5cccc6c(N(c7cccc(-c8cccc9ccccc89)c7)c7cccc(-c8cccc9ccccc89)c7)cccc56)cccc43)o2)cc1 ; c1ccc(-c2ccc(N(c3ccc(-c4cccc5ccccc45)cc3-c3ccccc3)c3ccc(-c4cc5oc(-c6ccccc6)nc5c5ccccc45)cc3-c3ccccc3)cc2)cc1]

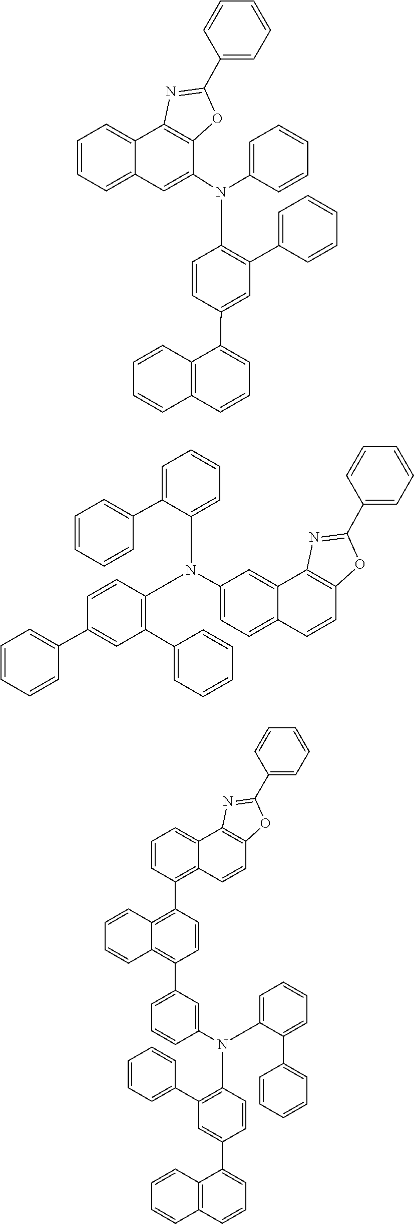 [SMILES: c1ccc(-c2nc3c(o2)c(N(c2ccccc2)c2ccc(-c4cccc5ccccc45)cc2-c2ccccc2)cc2ccccc23)cc1 ; c1ccc(-c2ccc(N(c3ccc4ccc5oc(-c6ccccc6)nc5c4c3)c3ccccc3-c3ccccc3)c(-c3ccccc3)c2)cc1 ; c1ccc(-c2nc3c(ccc4c(-c5ccc(-c6cccc(N(c7ccccc7-c7ccccc7)c7ccc(-c8cccc9ccccc89)cc7-c7ccccc7)c6)c6ccccc56)cccc43)o2)cc1]

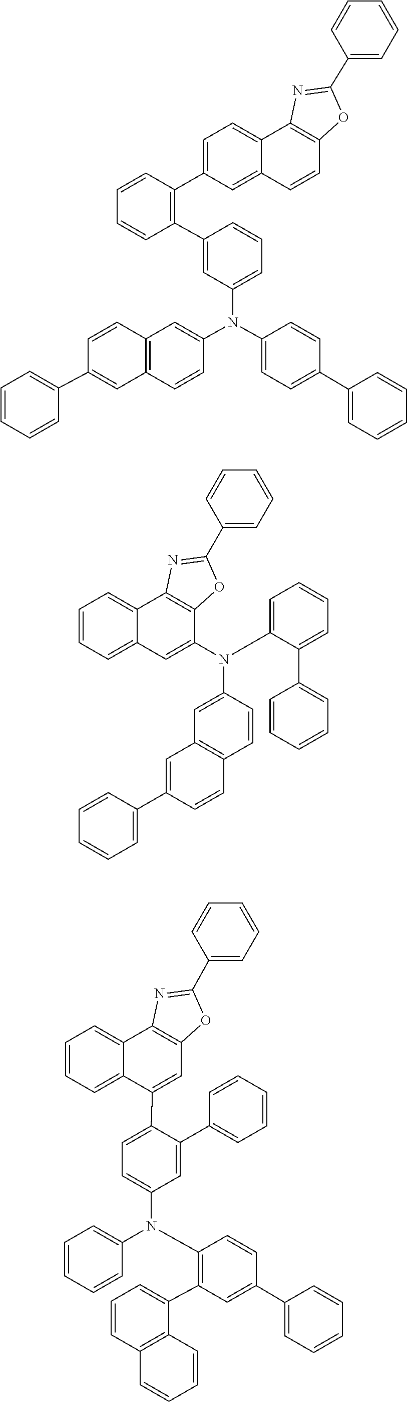 [SMILES: c1ccc(-c2ccc(N(c3cccc(-c4ccccc4-c4ccc5c(ccc6oc(-c7ccccc7)nc65)c4)c3)c3ccc4cc(-c5ccccc5)ccc4c3)cc2)cc1 ; c1ccc(-c2ccc3ccc(N(c4ccccc4-c4ccccc4)c4cc5ccccc5c5nc(-c6ccccc6)oc45)cc3c2)cc1 ; c1ccc(-c2ccc(N(c3ccccc3)c3ccc(-c4cc5oc(-c6ccccc6)nc5c5ccccc45)c(-c4ccccc4)c3)c(-c3cccc4ccccc34)c2)cc1]

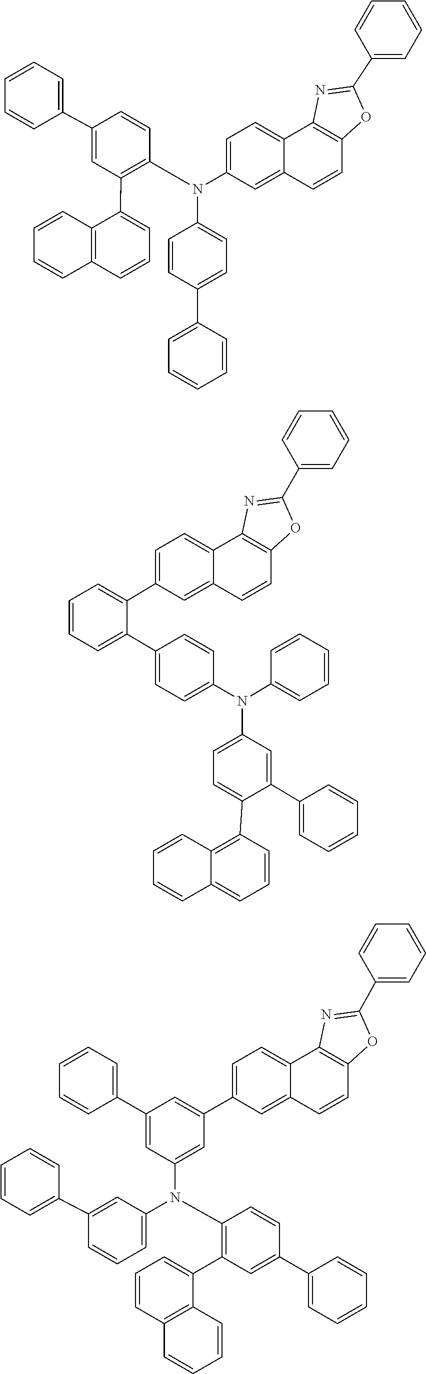 [SMILES: c1ccc(-c2ccc(N(c3ccc4c(ccc5oc(-c6ccccc6)nc54)c3)c3ccc(-c4ccccc4)cc3-c3cccc4ccccc34)cc2)cc1 ; c1ccc(-c2nc3c(ccc4cc(-c5ccccc5-c5ccc(N(c6ccccc6)c6ccc(-c7cccc8ccccc78)c(-c7ccccc7)c6)cc5)ccc43)o2)cc1 ; c1ccc(-c2cccc(N(c3cc(-c4ccccc4)cc(-c4ccc5c(ccc6oc(-c7ccccc7)nc65)c4)c3)c3ccc(-c4ccccc4)cc3-c3cccc4ccccc34)c2)cc1]

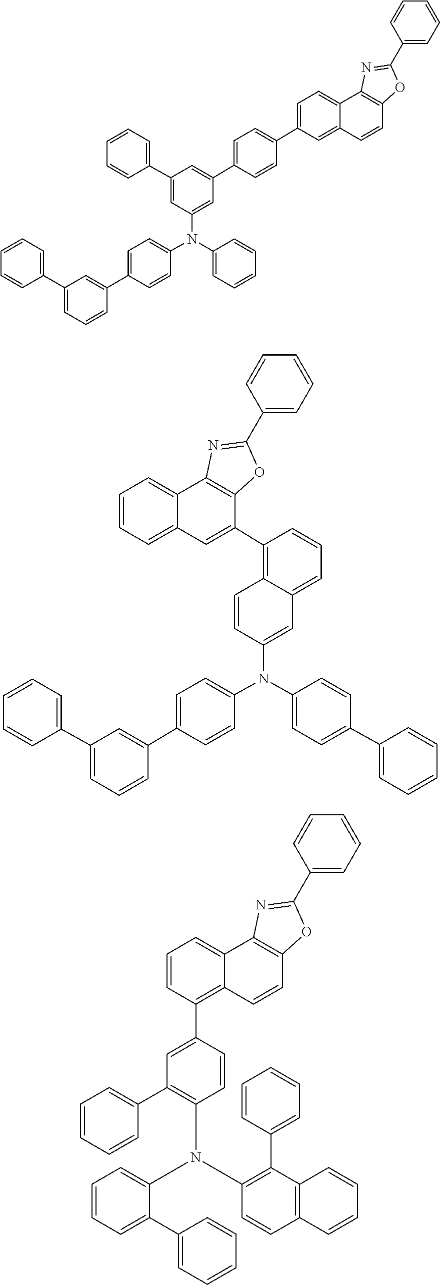 [SMILES: c1ccc(-c2cccc(-c3ccc(N(c4ccccc4)c4cc(-c5ccccc5)cc(-c5ccc(-c6ccc7c(ccc8oc(-c9ccccc9)nc87)c6)cc5)c4)cc3)c2)cc1 ; c1ccc(-c2ccc(N(c3ccc(-c4cccc(-c5ccccc5)c4)cc3)c3ccc4c(-c5cc6ccccc6c6nc(-c7ccccc7)oc56)cccc4c3)cc2)cc1 ; c1ccc(-c2nc3c(ccc4c(-c5ccc(N(c6ccccc6-c6ccccc6)c6ccc7ccccc7c6-c6ccccc6)c(-c6ccccc6)c5)cccc43)o2)cc1]

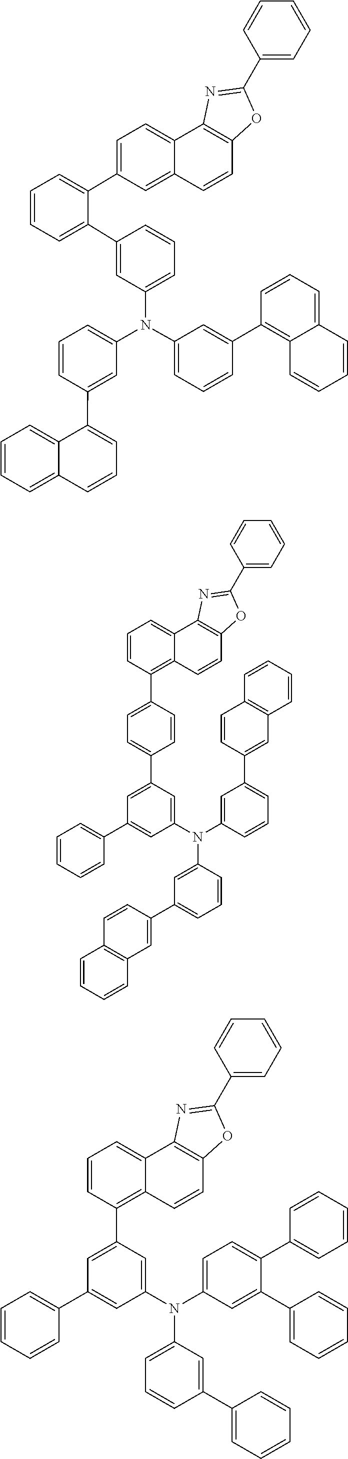 [SMILES: c1ccc(-c2nc3c(ccc4cc(-c5ccccc5-c5cccc(N(c6cccc(-c7cccc8ccccc78)c6)c6cccc(-c7cccc8ccccc78)c6)c5)ccc43)o2)cc1 ; c1ccc(-c2cc(-c3ccc(-c4cccc5c4ccc4oc(-c6ccccc6)nc45)cc3)cc(N(c3cccc(-c4ccc5ccccc5c4)c3)c3cccc(-c4ccc5ccccc5c4)c3)c2)cc1 ; c1ccc(-c2cccc(N(c3cc(-c4ccccc4)cc(-c4cccc5c4ccc4oc(-c6ccccc6)nc45)c3)c3ccc(-c4ccccc4)c(-c4ccccc4)c3)c2)cc1]

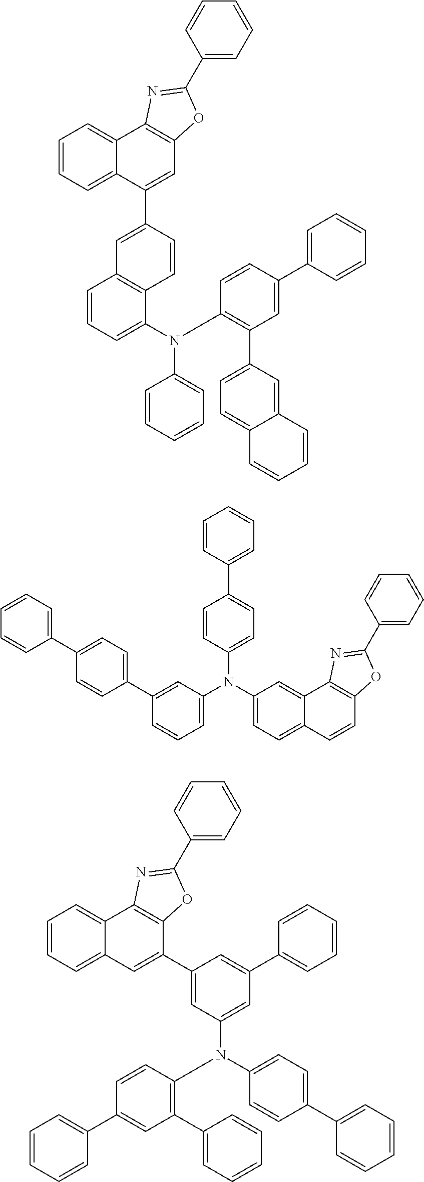

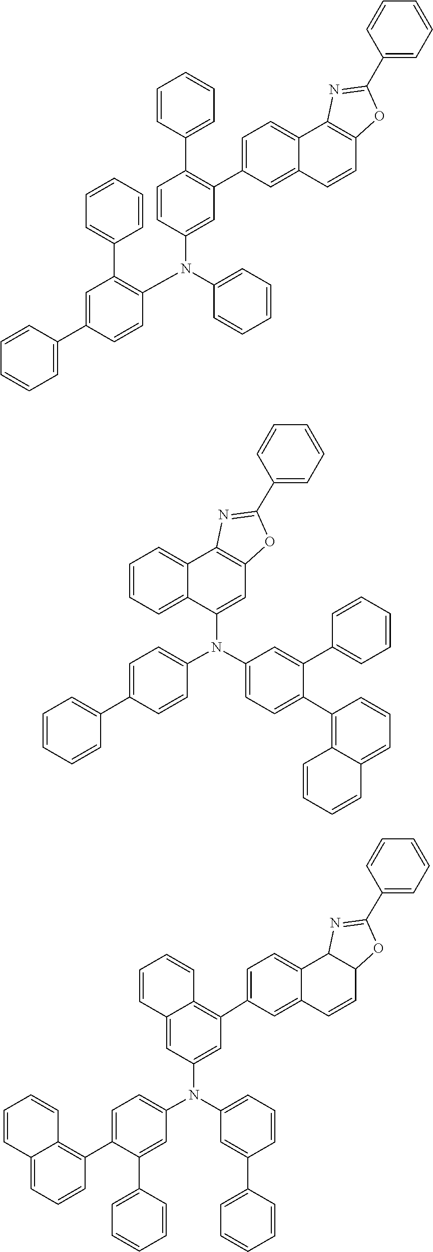 [SMILES: c1ccc(-c2ccc(N(c3ccccc3)c3ccc(-c4ccccc4)c(-c4ccc5c(ccc6oc(-c7ccccc7)nc65)c4)c3)c(-c3ccccc3)c2)cc1 ; c1ccc(-c2ccc(N(c3ccc(-c4cccc5ccccc45)c(-c4ccccc4)c3)c3cc4oc(-c5ccccc5)nc4c4ccccc34)cc2)cc1 ; C1=CC2OC(c3ccccc3)=NC2c2ccc(-c3cc(N(c4cccc(-c5ccccc5)c4)c4ccc(-c5cccc6ccccc56)c(-c5ccccc5)c4)cc4ccccc34)cc21]

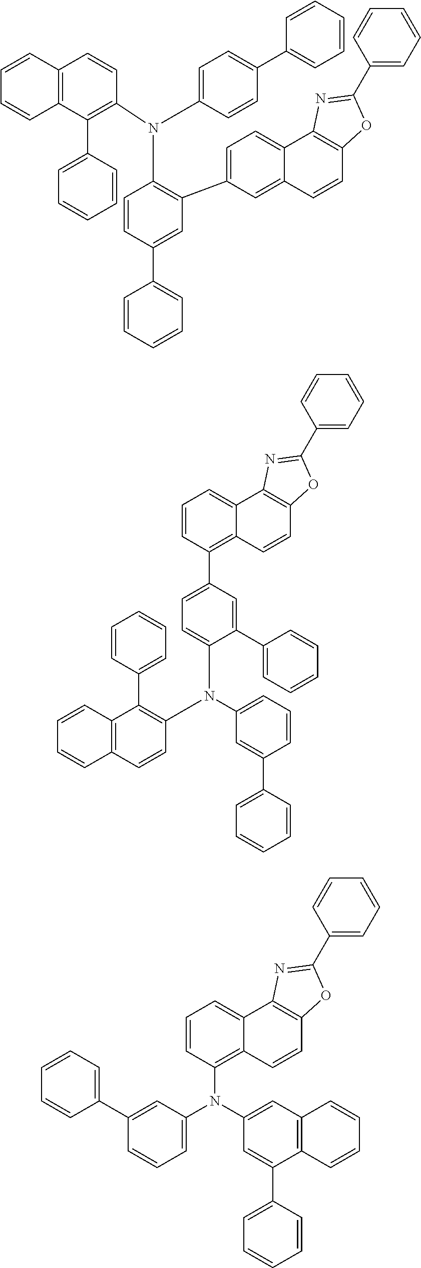 [SMILES: c1ccc(-c2ccc(N(c3ccc(-c4ccccc4)cc3-c3ccc4c(ccc5oc(-c6ccccc6)nc54)c3)c3ccc4ccccc4c3-c3ccccc3)cc2)cc1 ; c1ccc(-c2cccc(N(c3ccc(-c4cccc5c4ccc4oc(-c6ccccc6)nc45)cc3-c3ccccc3)c3ccc4ccccc4c3-c3ccccc3)c2)cc1 ; c1ccc(-c2cccc(N(c3cc(-c4ccccc4)c4ccccc4c3)c3cccc4c3ccc3oc(-c5ccccc5)nc34)c2)cc1]

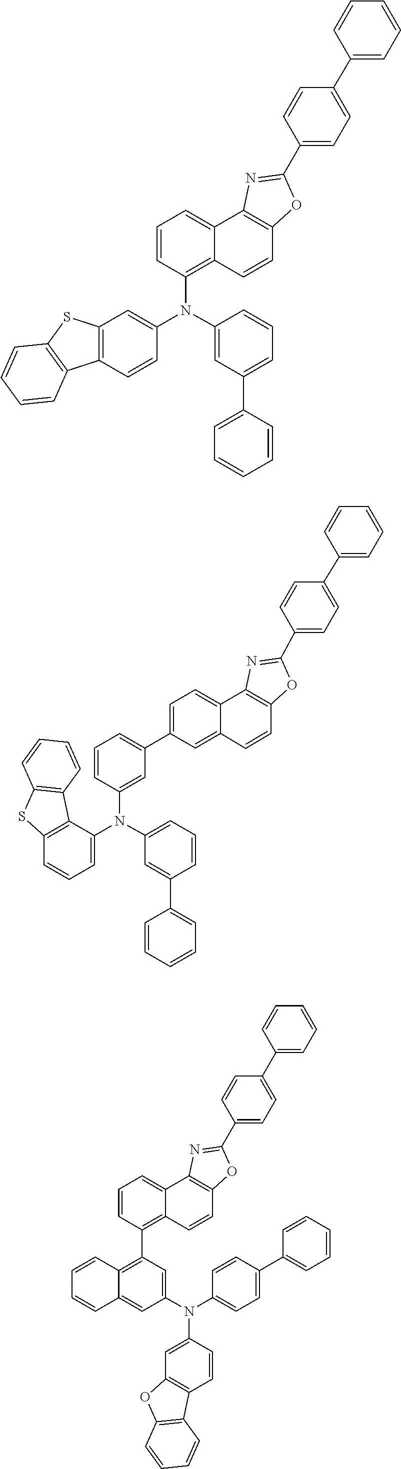 [SMILES: c1ccc(-c2ccc(-c3nc4c(ccc5c(N(c6cccc(-c7ccccc7)c6)c6ccc7c(c6)sc6ccccc67)cccc54)o3)cc2)cc1 ; c1ccc(-c2ccc(-c3nc4c(ccc5cc(-c6cccc(N(c7cccc(-c8ccccc8)c7)c7cccc8sc9ccccc9c78)c6)ccc54)o3)cc2)cc1 ; c1ccc(-c2ccc(-c3nc4c(ccc5c(-c6cc(N(c7ccc(-c8ccccc8)cc7)c7ccc8c(c7)oc7ccccc78)cc7ccccc67)cccc54)o3)cc2)cc1]

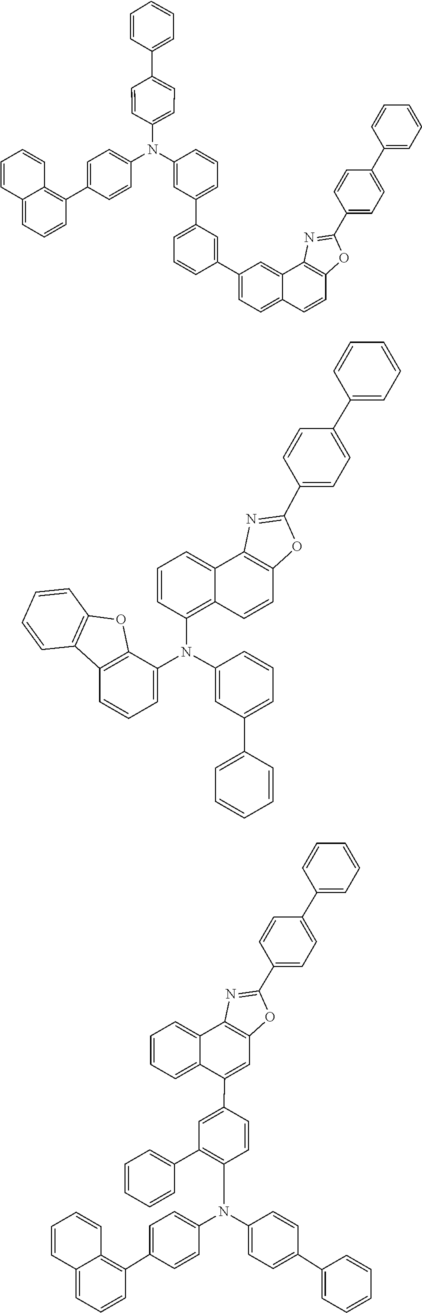 [SMILES: c1ccc(-c2ccc(-c3nc4c(ccc5ccc(-c6cccc(-c7cccc(N(c8ccc(-c9ccccc9)cc8)c8ccc(-c9cccc%10ccccc9%10)cc8)c7)c6)cc54)o3)cc2)cc1 ; c1ccc(-c2ccc(-c3nc4c(ccc5c(N(c6cccc(-c7ccccc7)c6)c6cccc7c6oc6ccccc67)cccc54)o3)cc2)cc1 ; c1ccc(-c2ccc(-c3nc4c(cc(-c5ccc(N(c6ccc(-c7ccccc7)cc6)c6ccc(-c7cccc8ccccc78)cc6)c(-c6ccccc6)c5)c5ccccc54)o3)cc2)cc1]

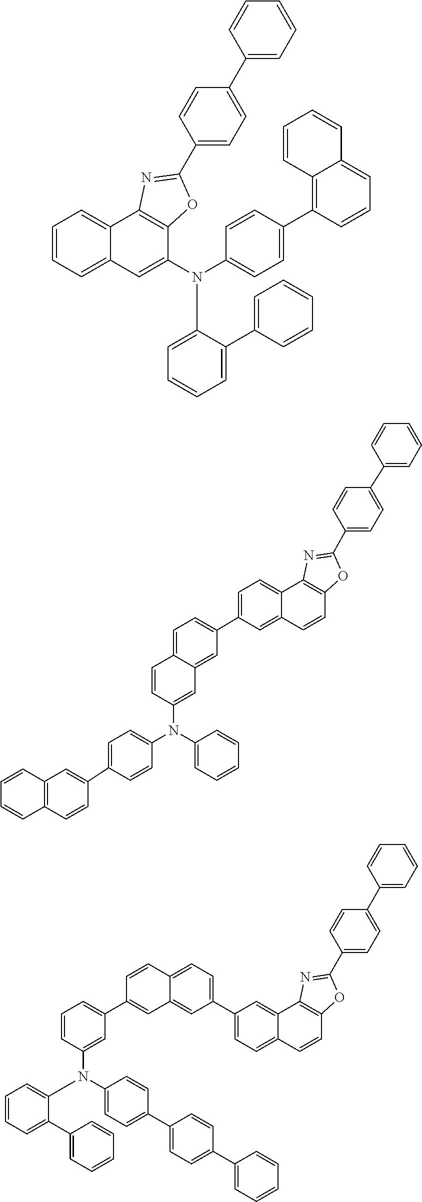 [SMILES: c1ccc(-c2ccc(-c3nc4c(o3)c(N(c3ccc(-c5cccc6ccccc56)cc3)c3ccccc3-c3ccccc3)cc3ccccc34)cc2)cc1 ; c1ccc(-c2ccc(-c3nc4c(ccc5cc(-c6ccc7ccc(N(c8ccccc8)c8ccc(-c9ccc%10ccccc%10c9)cc8)cc7c6)ccc54)o3)cc2)cc1 ; c1ccc(-c2ccc(-c3ccc(N(c4cccc(-c5ccc6ccc(-c7ccc8ccc9oc(-c%10ccc(-c%11ccccc%11)cc%10)nc9c8c7)cc6c5)c4)c4ccccc4-c4ccccc4)cc3)cc2)cc1]

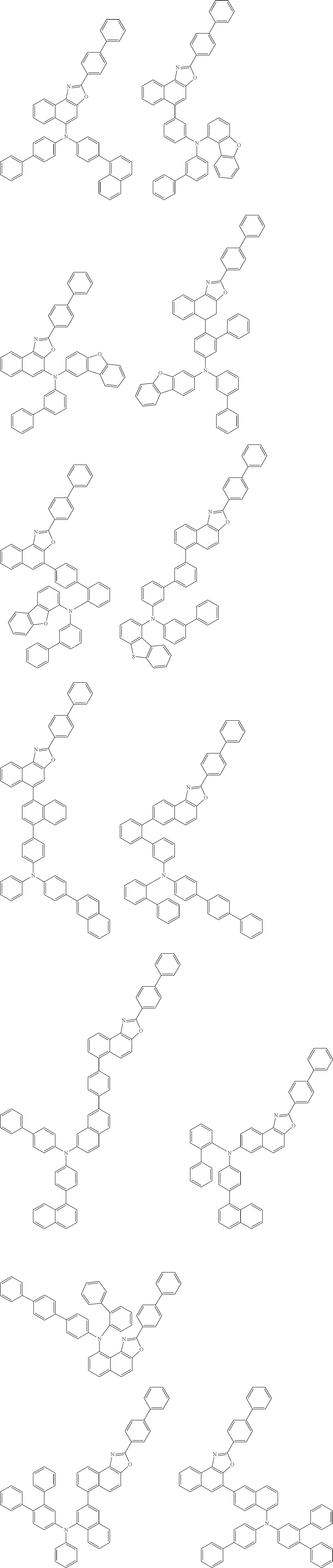

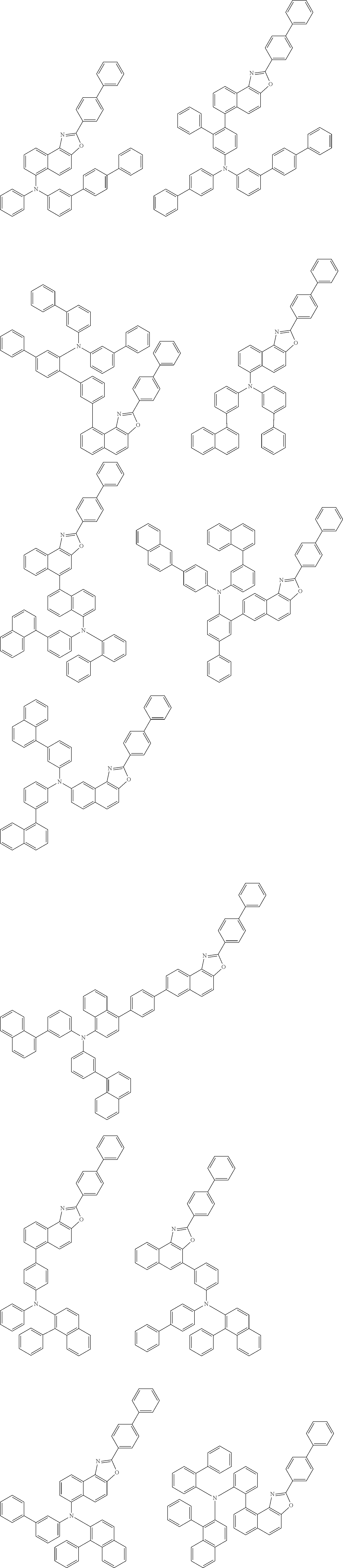

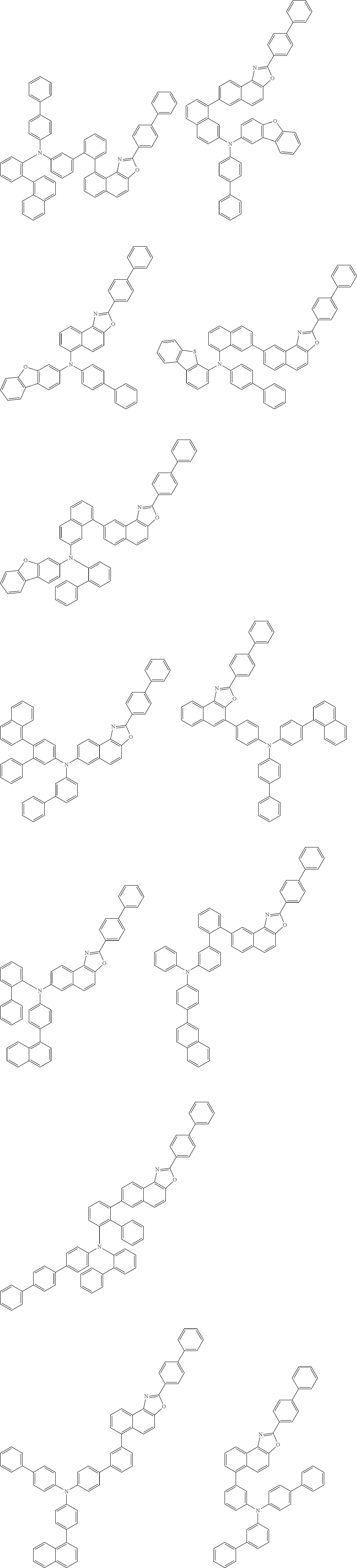

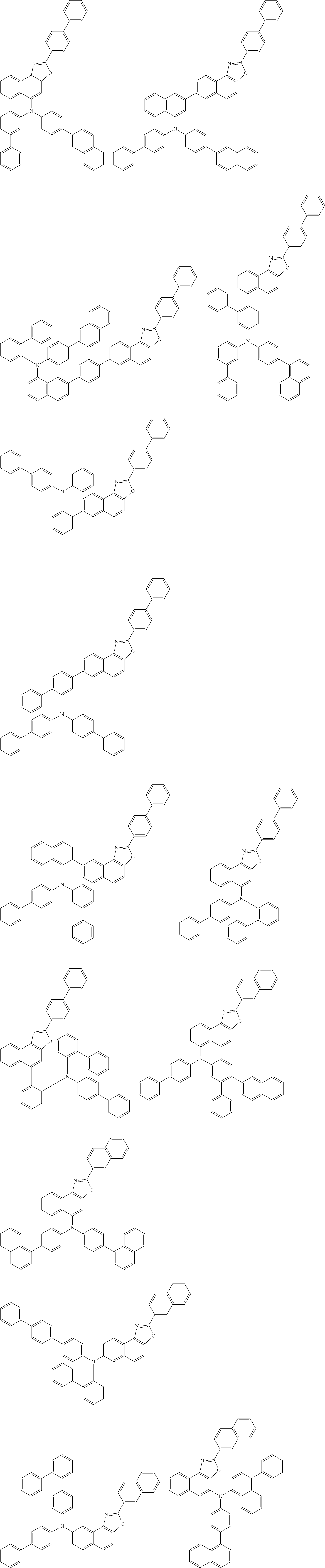

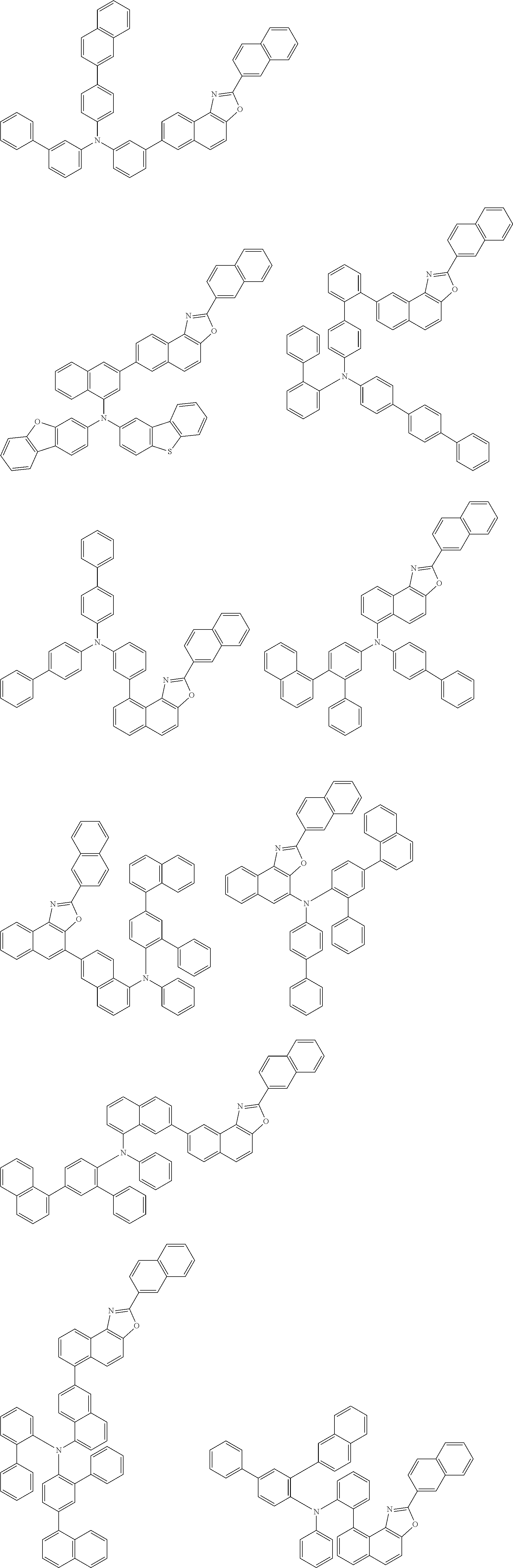

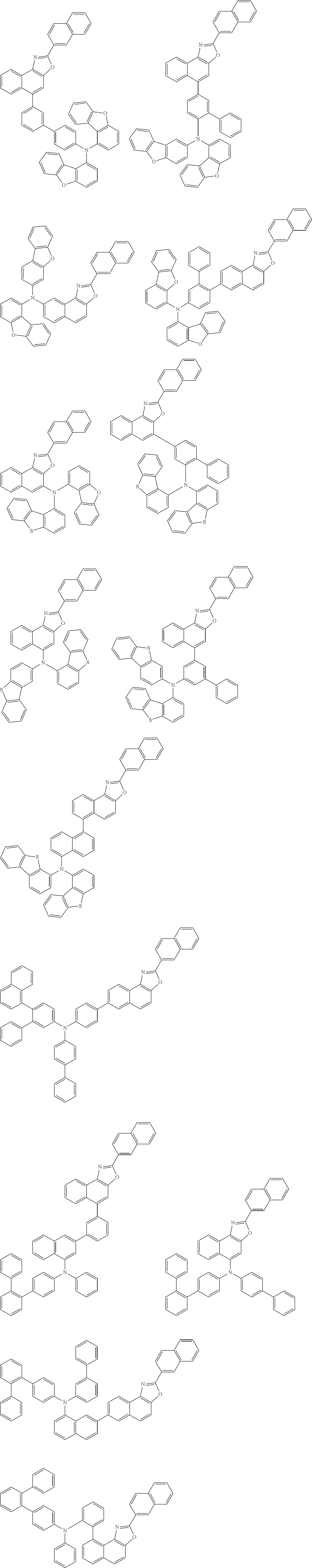

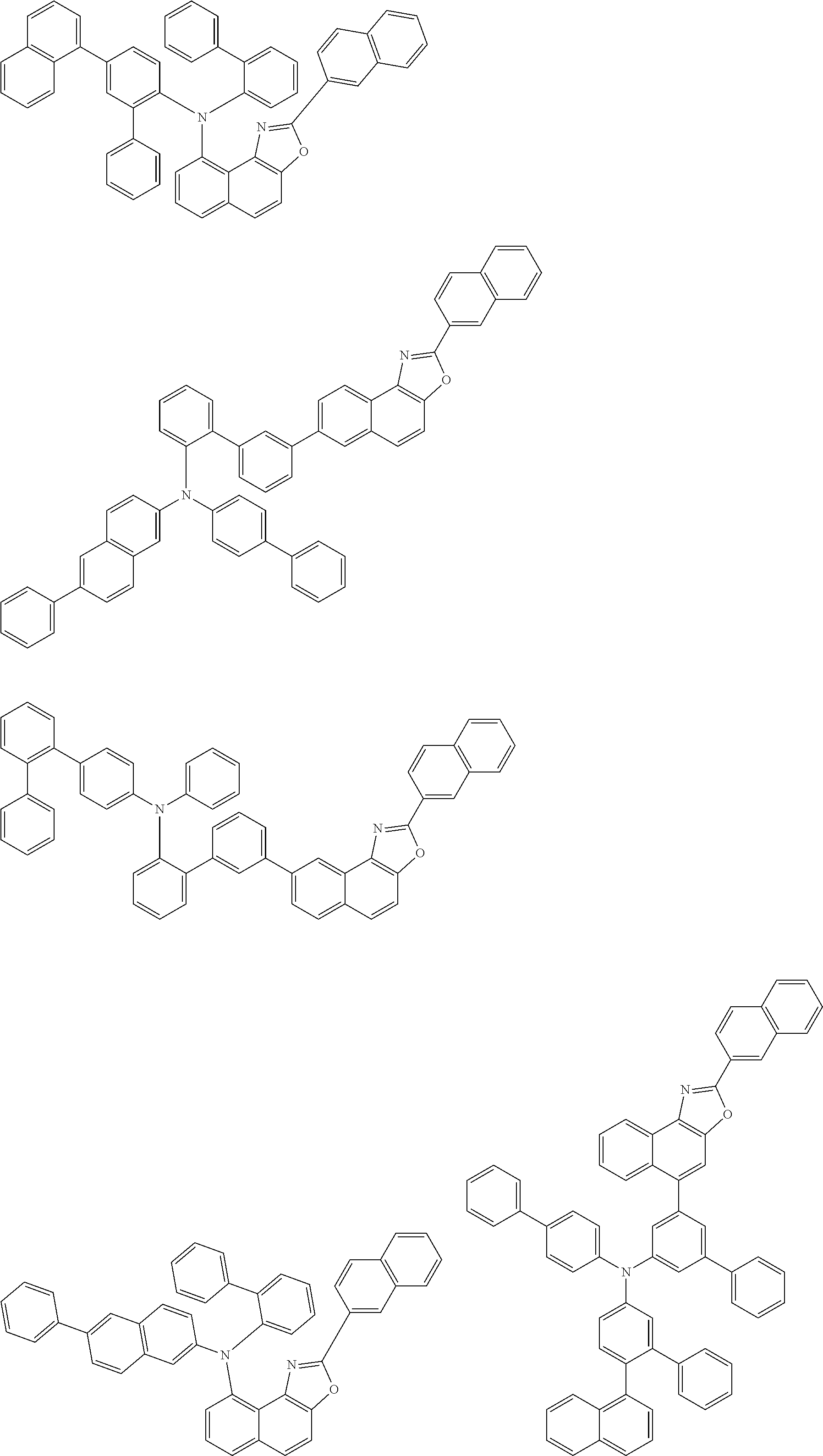

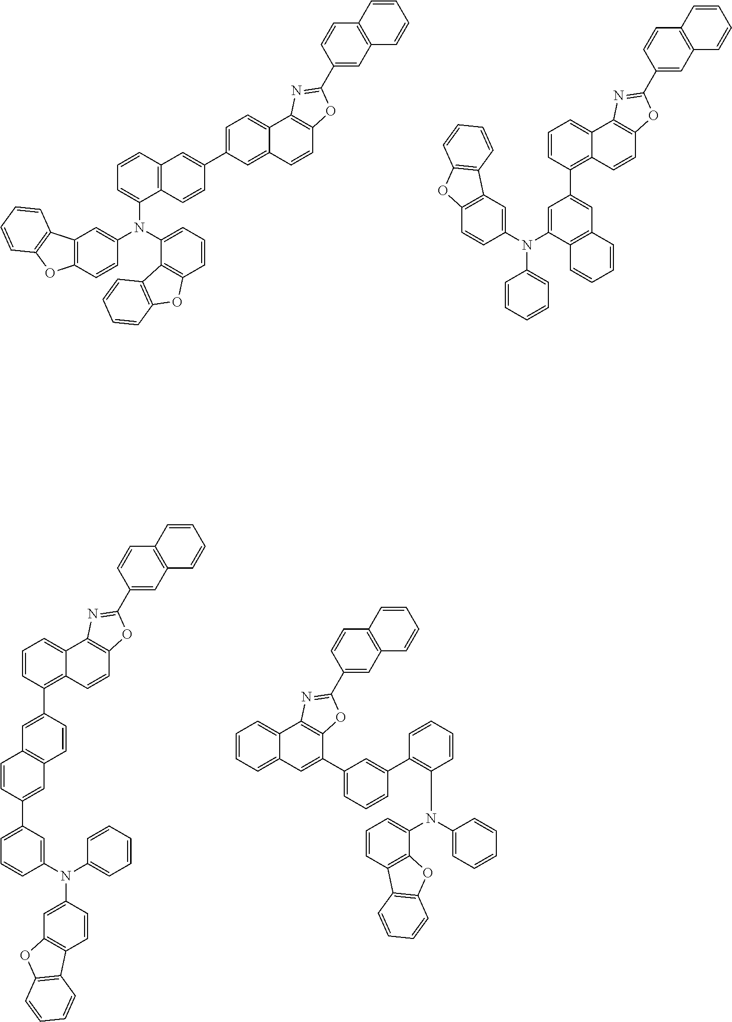 [SMILES: c1ccc2cc(-c3nc4c(ccc5cc(-c6ccc7c(N(c8ccc9oc%10ccccc%10c9c8)c8cccc9oc%10ccccc%10c89)cccc7c6)ccc54)o3)ccc2c1 ; c1ccc(N(c2ccc3oc4ccccc4c3c2)c2cc(-c3cccc4c3ccc3oc(-c5ccc6ccccc6c5)nc34)cc3ccccc23)cc1 ; c1ccc(N(c2cccc(-c3ccc4cc(-c5cccc6c5ccc5oc(-c7ccc8ccccc8c7)nc56)ccc4c3)c2)c2ccc3c(c2)oc2ccccc23)cc1 ; c1ccc(N(c2ccccc2-c2cccc(-c3cc4ccccc4c4nc(-c5ccc6ccccc6c5)oc34)c2)c2cccc3c2oc2ccccc23)cc1]

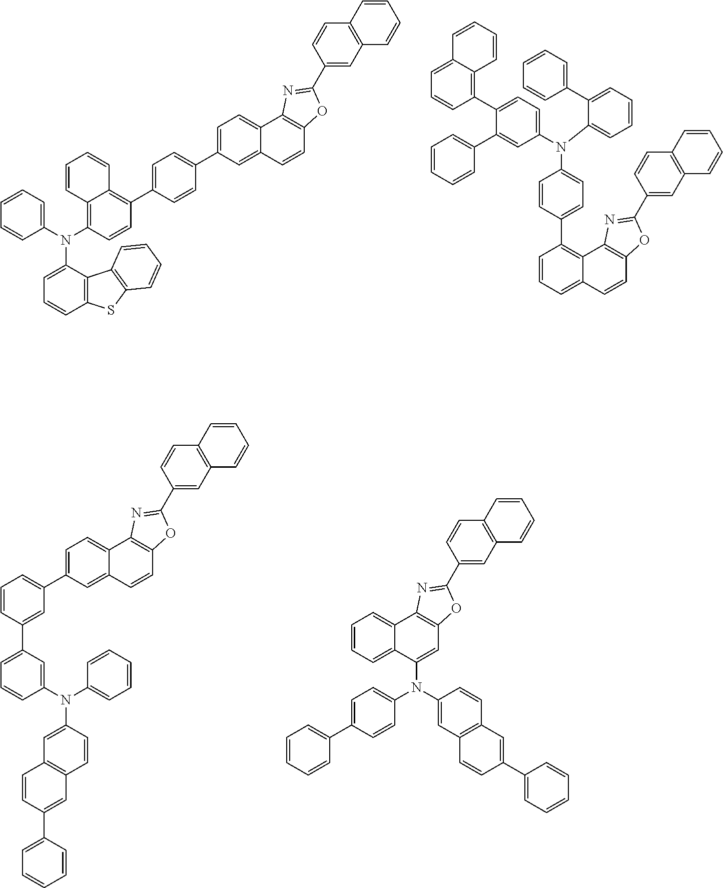 [SMILES: c1ccc(N(c2ccc(-c3ccc(-c4ccc5c(ccc6oc(-c7ccc8ccccc8c7)nc65)c4)cc3)c3ccccc23)c2cccc3sc4ccccc4c23)cc1 ; c1ccc(-c2cc(N(c3ccc(-c4cccc5ccc6oc(-c7ccc8ccccc8c7)nc6c45)cc3)c3ccccc3-c3ccccc3)ccc2-c2cccc3ccccc23)cc1 ; c1ccc(-c2ccc3cc(N(c4ccccc4)c4cccc(-c5cccc(-c6ccc7c(ccc8oc(-c9ccc%10ccccc%10c9)nc87)c6)c5)c4)ccc3c2)cc1 ; c1ccc(-c2ccc(N(c3ccc4cc(-c5ccccc5)ccc4c3)c3cc4oc(-c5ccc6ccccc6c5)nc4c4ccccc34)cc2)cc1]

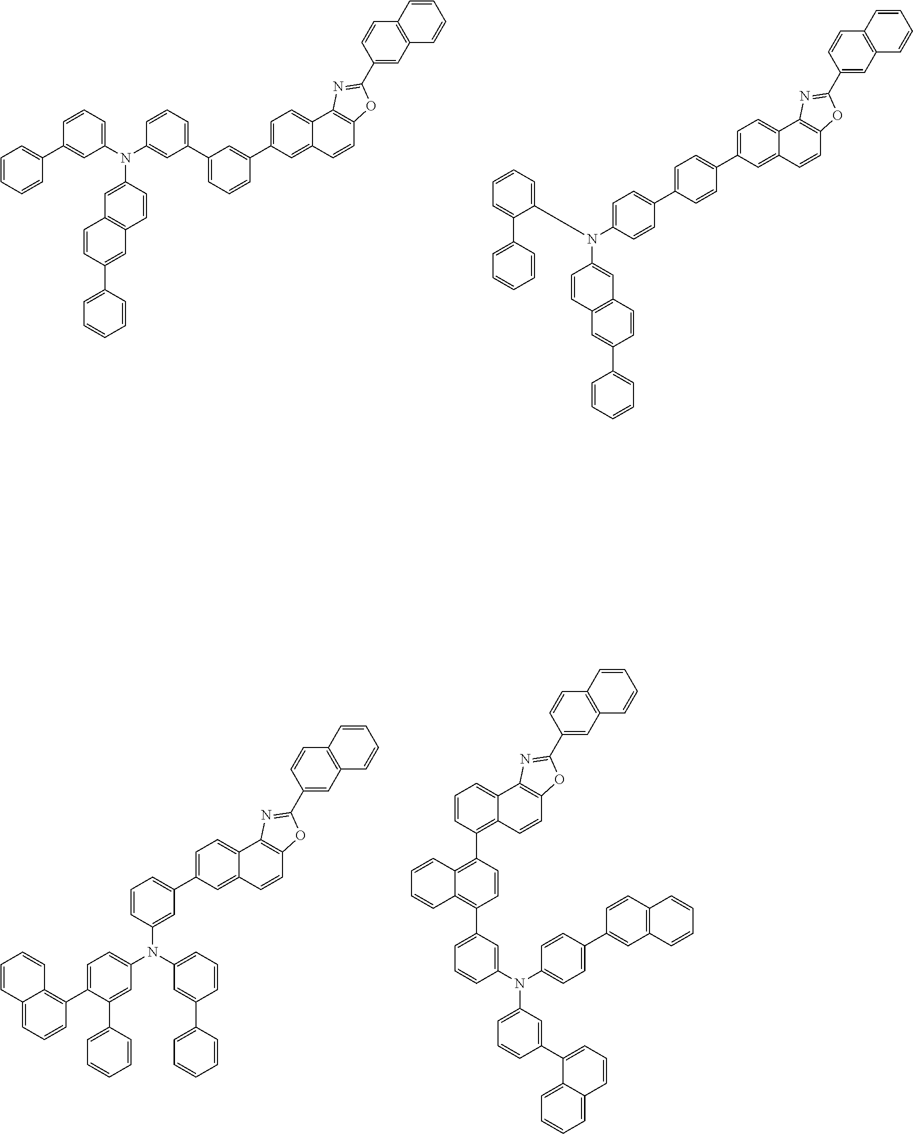 [SMILES: c1ccc(-c2cccc(N(c3cccc(-c4cccc(-c5ccc6c(ccc7oc(-c8ccc9ccccc9c8)nc76)c5)c4)c3)c3ccc4cc(-c5ccccc5)ccc4c3)c2)cc1 ; c1ccc(-c2ccc3cc(N(Cc4ccccc4-c4ccccc4)c4ccc(-c5ccc(-c6ccc7c(ccc8oc(-c9ccc%10ccccc%10c9)nc87)c6)cc5)cc4)ccc3c2)cc1 ; c1ccc(-c2cccc(N(c3cccc(-c4ccc5c(ccc6oc(-c7ccc8ccccc8c7)nc65)c4)c3)c3ccc(-c4cccc5ccccc45)c(-c4ccccc4)c3)c2)cc1 ; c1cc(-c2cccc3ccccc23)cc(N(c2ccc(-c3ccc4ccccc4c3)cc2)c2cccc(-c3ccc(-c4cccc5c4ccc4oc(-c6ccc7ccccc7c6)nc45)c4ccccc34)c2)c1]

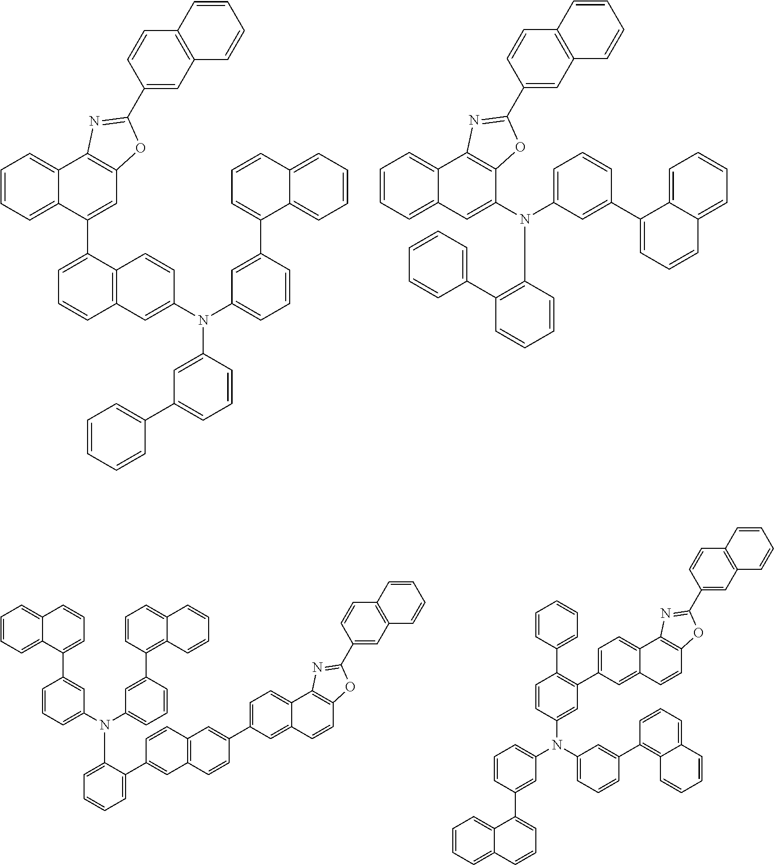

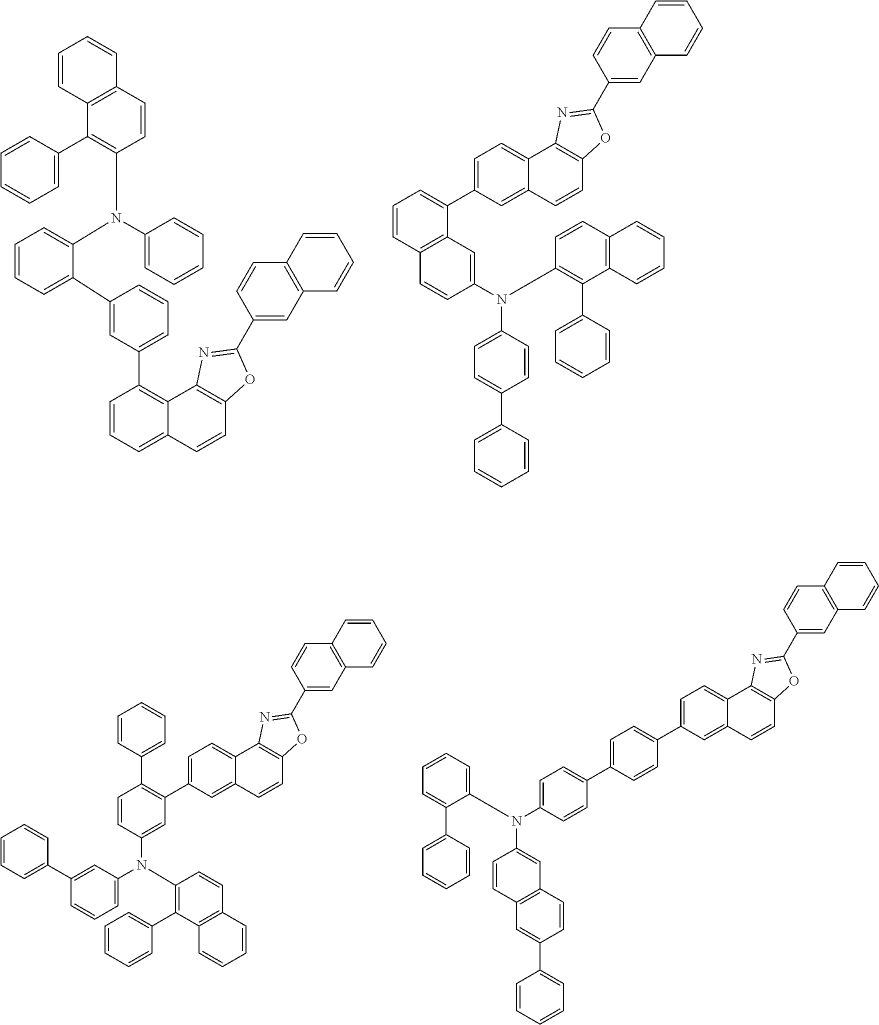 [SMILES: c1ccc(-c2c(N(c3ccccc3)c3ccccc3-c3cccc(-c4cccc5ccc6oc(-c7ccc8ccccc8c7)nc6c45)c3)ccc3ccccc23)cc1 ; c1ccc(-c2ccc(N(Cc3ccc4ccccc4c3-c3ccccc3)c3ccc4cccc(-c5ccc6c(ccc7oc(-c8ccc9ccccc9c8)nc76)c5)c4c3)cc2)cc1 ; c1ccc(-c2cccc(N(c3ccc(-c4ccccc4)c(-c4ccc5c(ccc6oc(-c7ccc8ccccc8c7)nc65)c4)c3)c3ccc4ccccc4c3-c3ccccc3)c2)cc1 ; c1ccc(-c2ccc3cc(N(Cc4ccccc4-c4ccccc4)c4ccc(-c5ccc(-c6ccc7c(ccc8oc(-c9ccc%10ccccc%10c9)nc87)c6)cc5)cc4)ccc3c2)cc1]

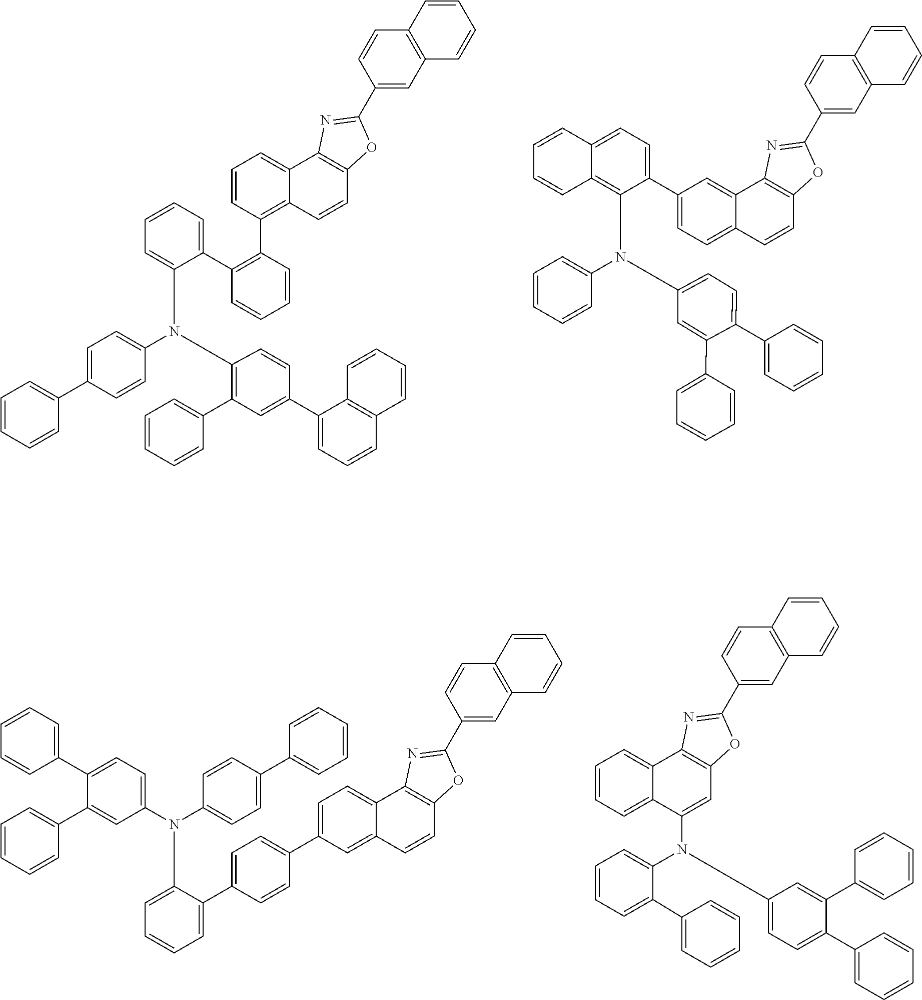 [SMILES: c1ccc(-c2ccc(N(Cc3ccc(-c4cccc5ccccc45)cc3-c3ccccc3)c3ccccc3-c3ccccc3-c3cccc4c3ccc3oc(-c5ccc6ccccc6c5)nc34)cc2)cc1 ; c1ccc(-c2ccc(CN(c3ccccc3)c3c(-c4ccc5ccc6oc(-c7ccc8ccccc8c7)nc6c5c4)ccc4ccccc34)cc2-c2ccccc2)cc1 ; c1ccc(-c2ccc(N(c3ccc(-c4ccccc4)c(-c4ccccc4)c3)c3ccccc3-c3ccc(-c4ccc5c(ccc6oc(-c7ccc8ccccc8c7)nc65)c4)cc3)cc2)cc1 ; c1ccc(-c2ccc(CCN(c3ccccc3-c3ccccc3)c3cc4oc(-c5ccc6ccccc6c5)nc4c4ccccc34)cc2-c2ccccc2)cc1]

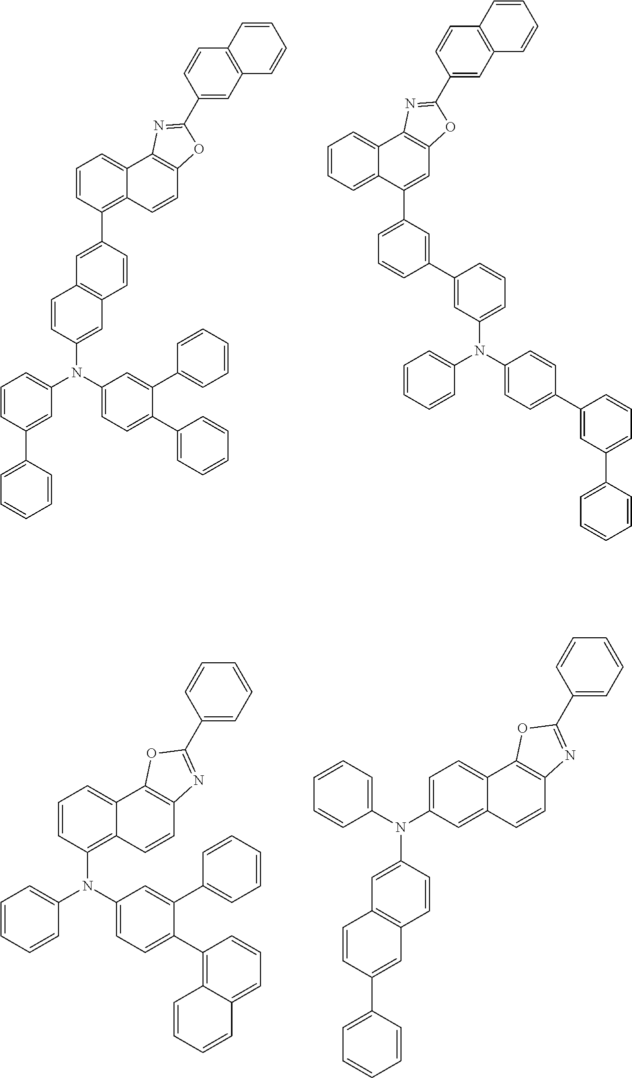 [SMILES: c1ccc(-c2cccc(N(c3ccc(-c4ccccc4)c(-c4ccccc4)c3)c3ccc4cc(-c5cccc6c5ccc5oc(-c7ccc8ccccc8c7)nc56)ccc4c3)c2)cc1 ; c1ccc(-c2cccc(-c3ccc(N(c4ccccc4)c4cccc(-c5cccc(-c6cc7oc(-c8ccc9ccccc9c8)nc7c7ccccc67)c5)c4)cc3)c2)cc1 ; c1ccc(-c2nc3ccc4c(N(c5ccccc5)c5ccc(-c6cccc7ccccc67)c(-c6ccccc6)c5)cccc4c3o2)cc1 ; c1ccc(-c2ccc3cc(N(c4ccccc4)c4ccc5c(ccc6nc(-c7ccccc7)oc65)c4)ccc3c2)cc1]

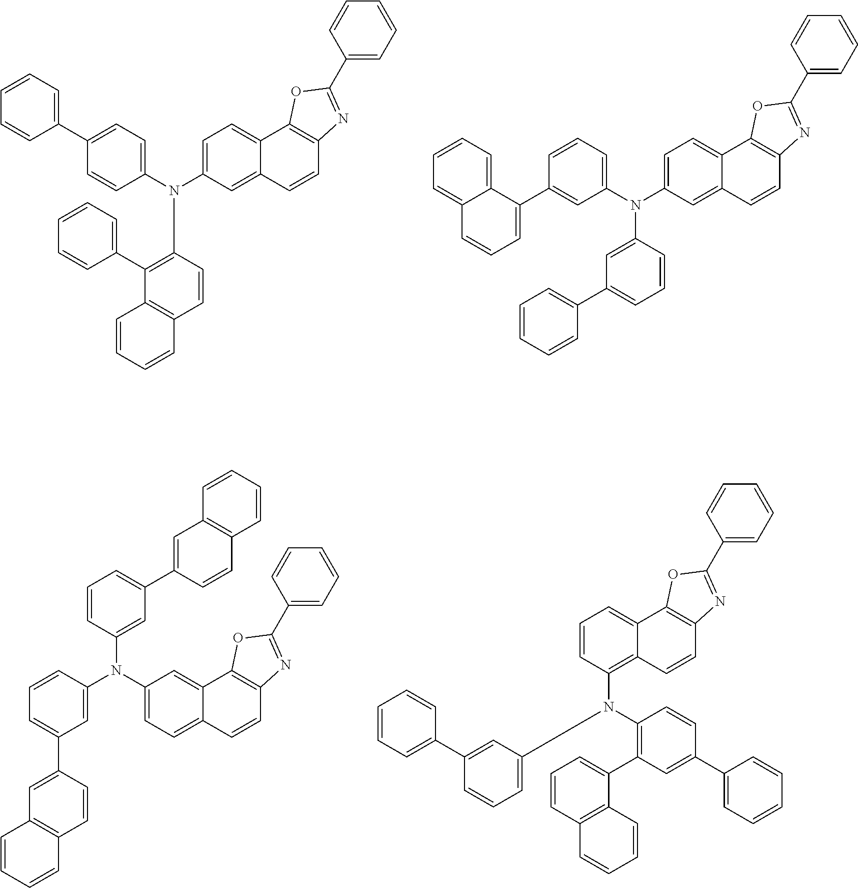 [SMILES: c1ccc(-c2ccc(N(c3ccc4c(ccc5nc(-c6ccccc6)oc54)c3)c3ccc4ccccc4c3-c3ccccc3)cc2)cc1 ; c1ccc(-c2cccc(N(c3cccc(-c4cccc5ccccc45)c3)c3ccc4c(ccc5nc(-c6ccccc6)oc54)c3)c2)cc1 ; c1ccc(-c2nc3ccc4ccc(N(c5cccc(-c6ccc7ccccc7c6)c5)c5cccc(-c6ccc7ccccc7c6)c5)cc4c3o2)cc1 ; c1ccc(-c2cccc(CCN(c3ccc(-c4ccccc4)cc3-c3cccc4ccccc34)c3cccc4c3ccc3nc(-c5ccccc5)oc34)c2)cc1]

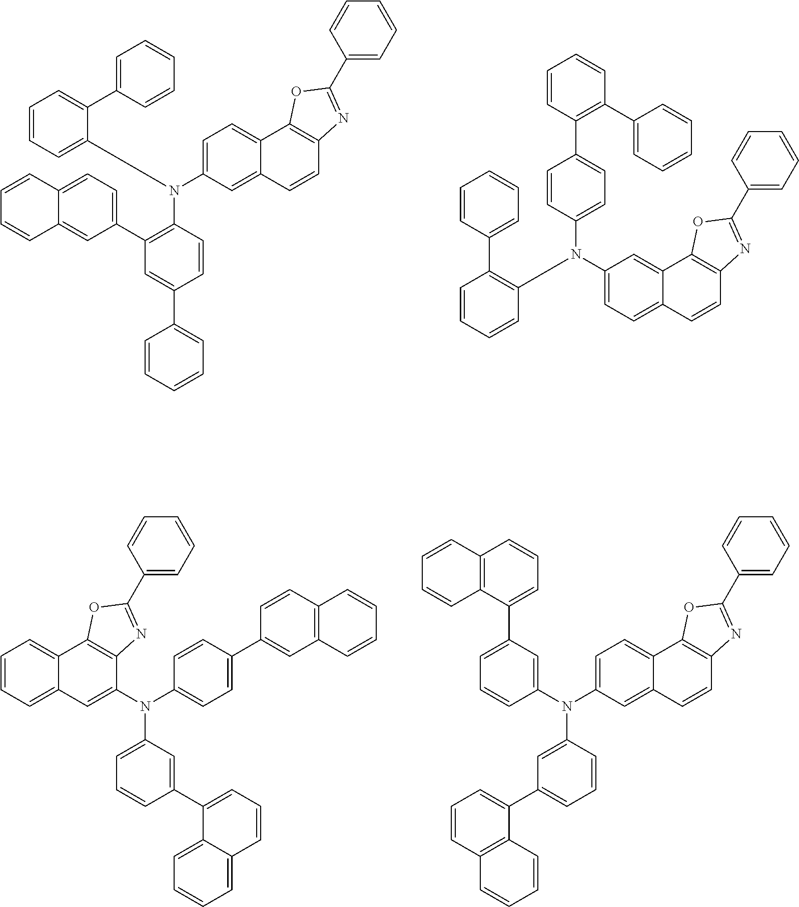 [SMILES: c1ccc(-c2ccc(N(CCc3ccccc3-c3ccccc3)c3ccc4c(ccc5nc(-c6ccccc6)oc54)c3)c(-c3ccc4ccccc4c3)c2)cc1 ; c1ccc(-c2nc3ccc4ccc(N(Cc5ccccc5-c5ccccc5)c5ccc(-c6ccccc6-c6ccccc6)cc5)cc4c3o2)cc1 ; c1ccc(-c2nc3c(N(c4ccc(-c5ccc6ccccc6c5)cc4)c4cccc(-c5cccc6ccccc56)c4)cc4ccccc4c3o2)cc1 ; c1ccc(-c2nc3ccc4cc(N(c5cccc(-c6cccc7ccccc67)c5)c5cccc(-c6cccc7ccccc67)c5)ccc4c3o2)cc1]

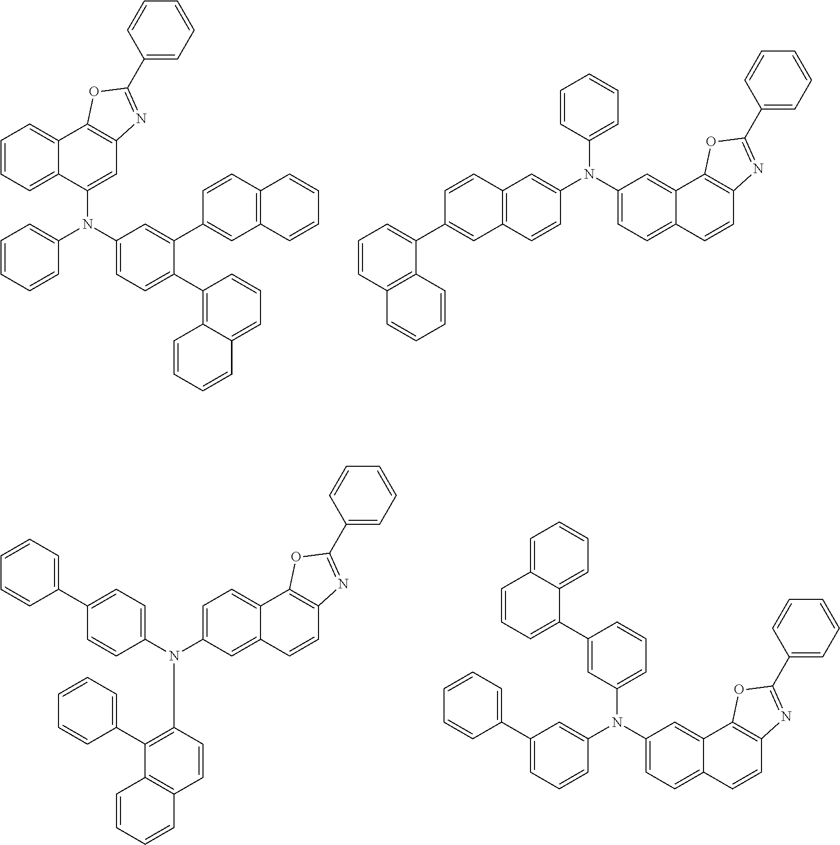

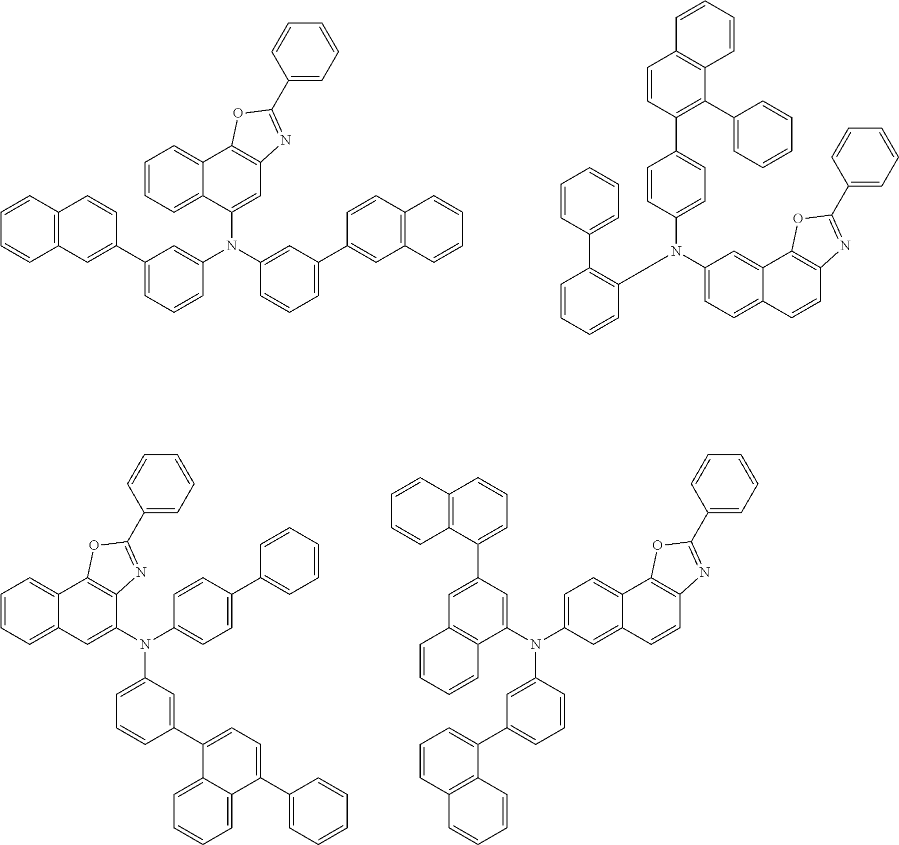 [SMILES: c1ccc(-c2nc3cc(N(c4cccc(-c5ccc6ccccc6c5)c4)c4cccc(-c5ccc6ccccc6c5)c4)c4ccccc4c3o2)cc1 ; c1ccc(-c2nc3ccc4ccc(N(Cc5ccccc5-c5ccccc5)c5ccc(-c6ccc7ccccc7c6-c6ccccc6)cc5)cc4c3o2)cc1 ; c1ccc(-c2ccc(N(c3cccc(-c4ccc(-c5ccccc5)c5ccccc45)c3)c3cc4ccccc4c4oc(-c5ccccc5)nc34)cc2)cc1 ; c1ccc(-c2nc3ccc4cc(N(c5cccc(-c6cccc7ccccc67)c5)c5cc(-c6cccc7ccccc67)cc6ccccc56)ccc4c3o2)cc1]

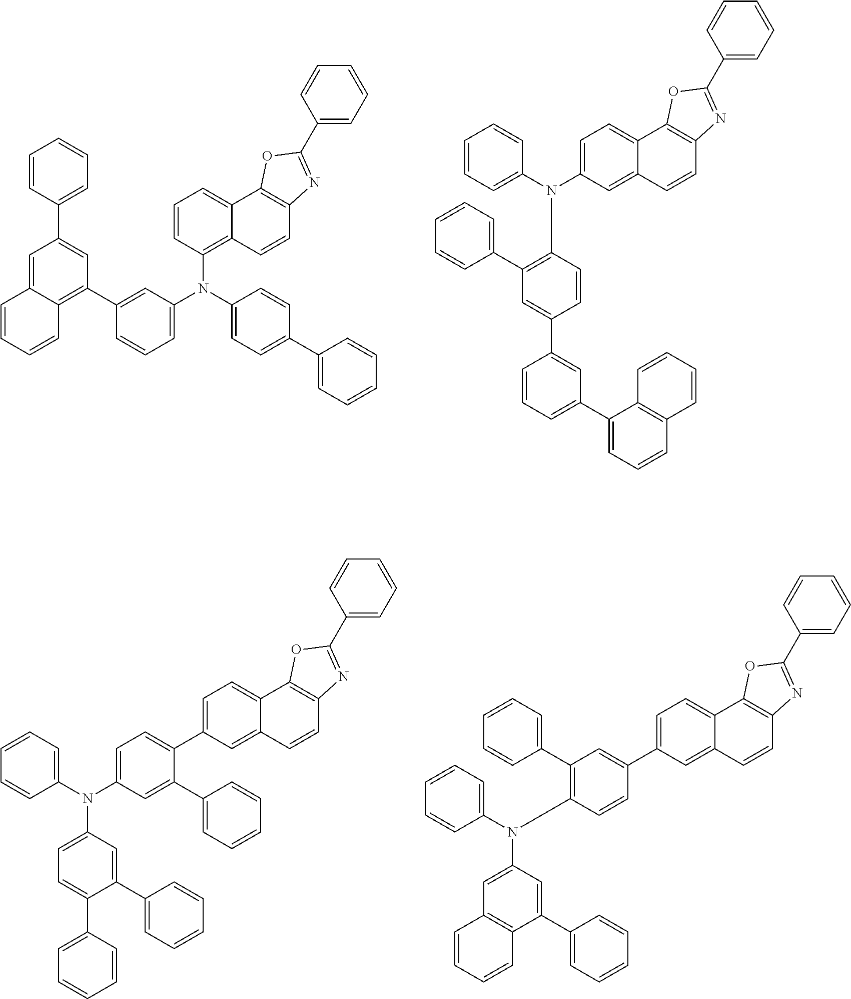 [SMILES: c1ccc(-c2ccc(N(c3cccc(-c4cc(-c5ccccc5)cc5ccccc45)c3)c3cccc4c3ccc3nc(-c5ccccc5)oc34)cc2)cc1 ; c1ccc(-c2nc3ccc4cc(N(c5ccccc5)c5ccc(-c6cccc(-c7cccc8ccccc78)c6)cc5-c5ccccc5)ccc4c3o2)cc1 ; c1ccc(-c2nc3ccc4cc(-c5ccc(N(c6ccccc6)c6ccc(-c7ccccc7)c(-c7ccccc7)c6)cc5-c5ccccc5)ccc4c3o2)cc1 ; c1ccc(-c2nc3ccc4cc(-c5ccc(CN(c6ccccc6)c6cc(-c7ccccc7)c7ccccc7c6)c(-c6ccccc6)c5)ccc4c3o2)cc1]

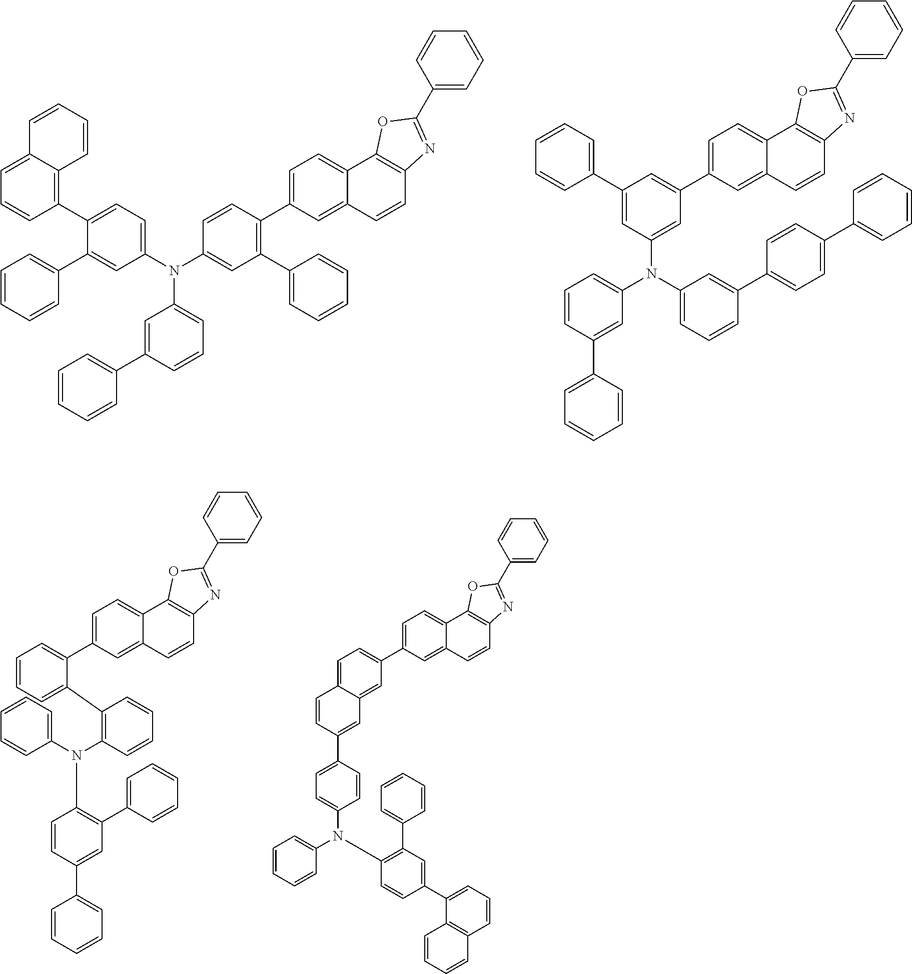 [SMILES: c1ccc(-c2cccc(N(c3ccc(-c4ccc5c(ccc6nc(-c7ccccc7)oc65)c4)c(-c4ccccc4)c3)c3ccc(-c4cccc5ccccc45)c(-c4ccccc4)c3)c2)cc1 ; c1ccc(-c2ccc(-c3cccc(N(c4cccc(-c5ccccc5)c4)c4cc(-c5ccccc5)cc(-c5ccc6c(ccc7nc(-c8ccccc8)oc76)c5)c4)c3)cc2)cc1 ; c1ccc(-c2ccc(N(c3ccccc3)c3ccccc3-c3ccccc3-c3ccc4c(ccc5nc(-c6ccccc6)oc54)c3)c(-c3ccccc3)c2)cc1 ; c1ccc(-c2nc3ccc4cc(-c5ccc6ccc(-c7ccc(N(Cc8ccc(-c9cccc%10ccccc9%10)cc8-c8ccccc8)c8ccccc8)cc7)cc6c5)ccc4c3o2)cc1]

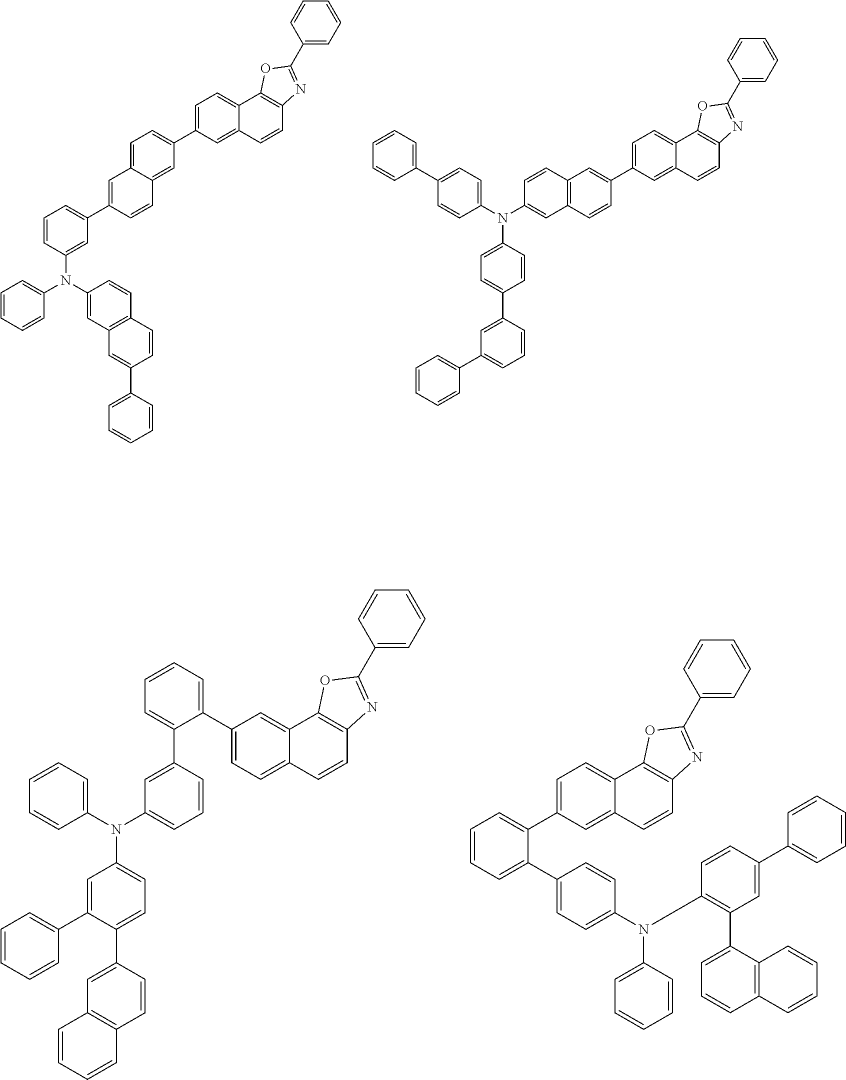 [SMILES: c1ccc(-c2ccc3ccc(N(c4ccccc4)c4cccc(-c5ccc6cc(-c7ccc8c(ccc9nc(-c%10ccccc%10)oc98)c7)ccc6c5)c4)cc3c2)cc1 ; c1ccc(-c2ccc(N(c3ccc(-c4cccc(-c5ccccc5)c4)cc3)c3ccc4cc(-c5ccc6c(ccc7nc(-c8ccccc8)oc76)c5)ccc4c3)cc2)cc1 ; c1ccc(-c2nc3ccc4ccc(-c5ccccc5-c5cccc(N(c6ccccc6)c6ccc(-c7ccc8ccccc8c7)c(-c7ccccc7)c6)c5)cc4c3o2)cc1 ; c1ccc(-c2ccc(CN(c3ccccc3)c3ccc(-c4ccccc4-c4ccc5c(ccc6nc(-c7ccccc7)oc65)c4)cc3)c(-c3cccc4ccccc34)c2)cc1]

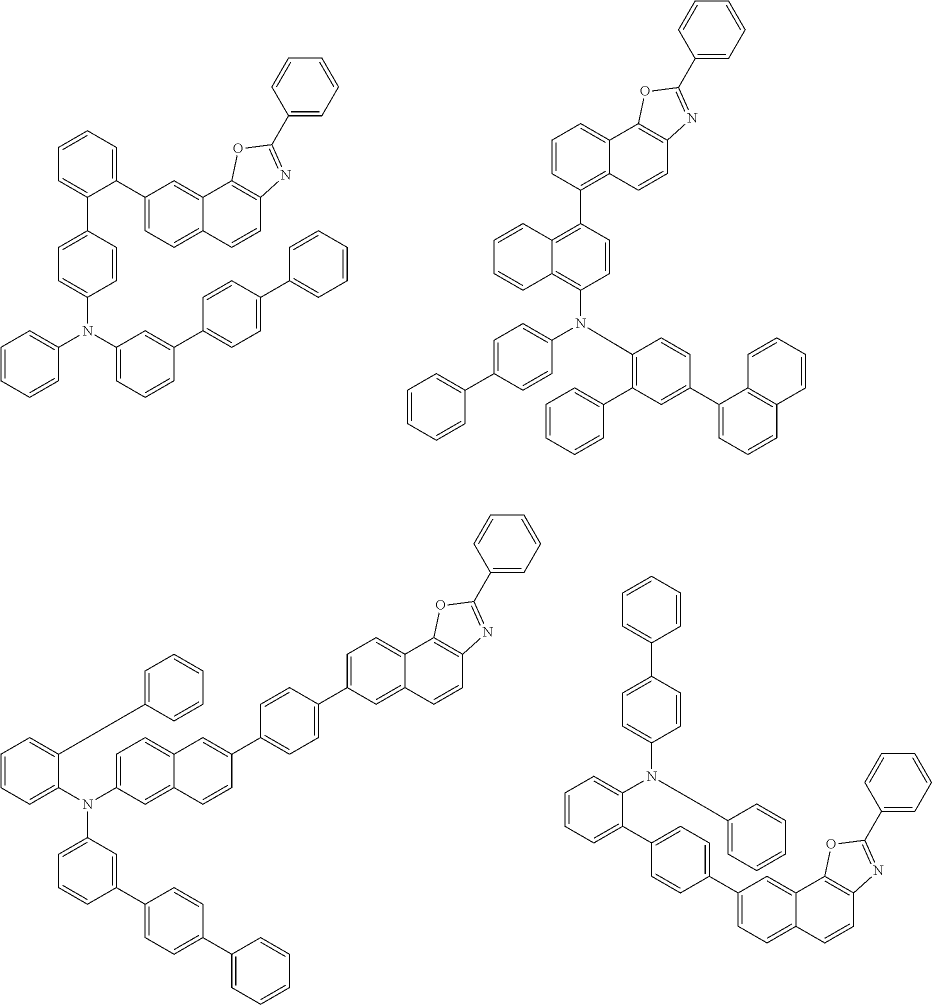 [SMILES: c1ccc(-c2ccc(-c3cccc(N(c4ccccc4)c4ccc(-c5ccccc5-c5ccc6ccc7nc(-c8ccccc8)oc7c6c5)cc4)c3)cc2)cc1 ; c1ccc(-c2ccc(N(c3ccc(-c4cccc5ccccc45)cc3-c3ccccc3)c3ccc(-c4cccc5c4ccc4nc(-c6ccccc6)oc45)c4ccccc34)cc2)cc1 ; c1ccc(CCc2ccccc2N(c2cccc(-c3ccc(-c4ccccc4)cc3)c2)c2ccc3cc(-c4ccc(-c5ccc6c(ccc7nc(-c8ccccc8)oc76)c5)cc4)ccc3c2)cc1 ; c1ccc(CCN(c2ccc(-c3ccccc3)cc2)c2ccccc2-c2ccc(-c3ccc4ccc5nc(-c6ccccc6)oc5c4c3)cc2)cc1]

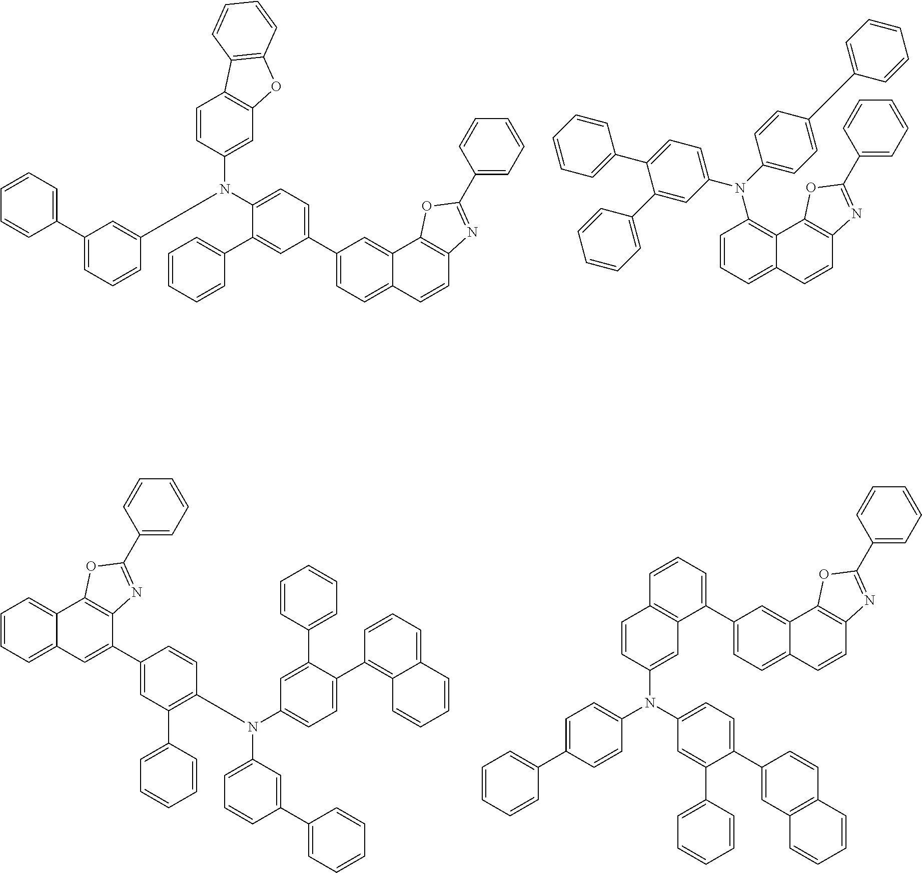

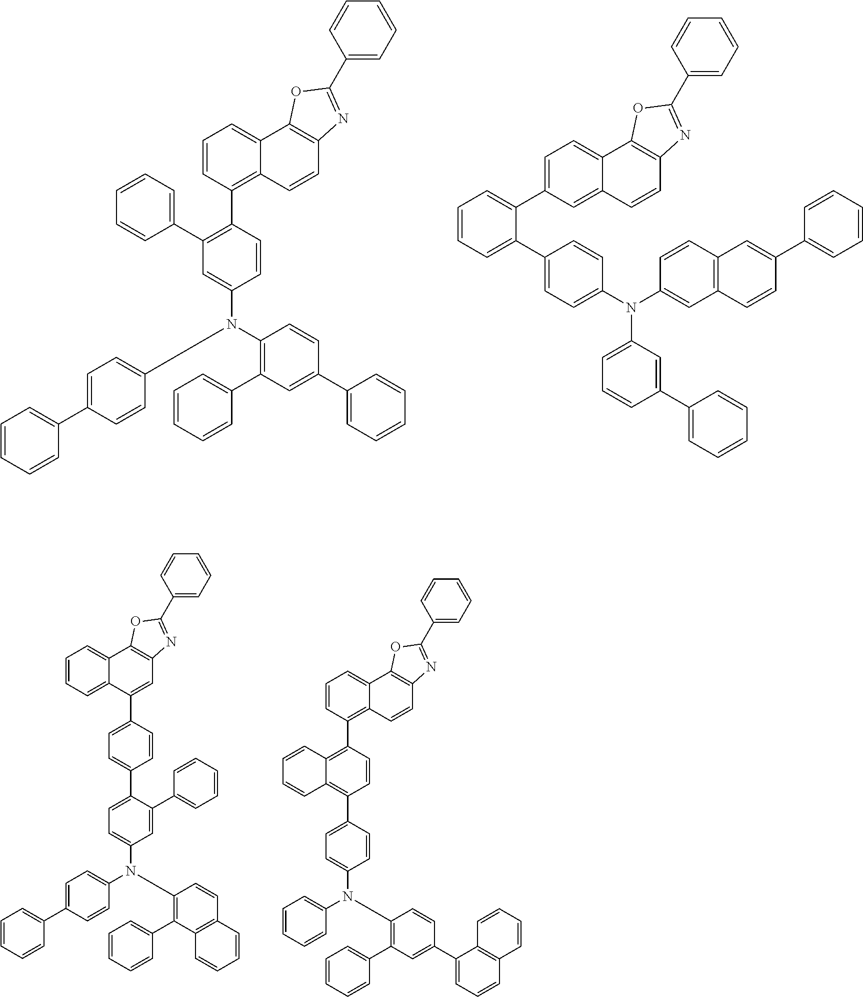 [SMILES: c1ccc(-c2ccc(CCN(c3ccc(-c4cccc5c4ccc4nc(-c6ccccc6)oc45)c(-c4ccccc4)c3)c3ccc(-c4ccccc4)cc3-c3ccccc3)cc2)cc1 ; c1ccc(-c2cccc(N(c3ccc(-c4ccccc4-c4ccc5c(ccc6nc(-c7ccccc7)oc65)c4)cc3)c3ccc4cc(-c5ccccc5)ccc4c3)c2)cc1 ; c1ccc(-c2ccc(N(Cc3ccc4ccccc4c3-c3ccccc3)c3ccc(-c4ccc(-c5cc6nc(-c7ccccc7)oc6c6ccccc56)cc4)c(-c4ccccc4)c3)cc2)cc1 ; c1ccc(-c2nc3ccc4c(-c5ccc(-c6ccc(N(Cc7ccc(-c8cccc9ccccc89)cc7-c7ccccc7)c7ccccc7)cc6)c6ccccc56)cccc4c3o2)cc1]

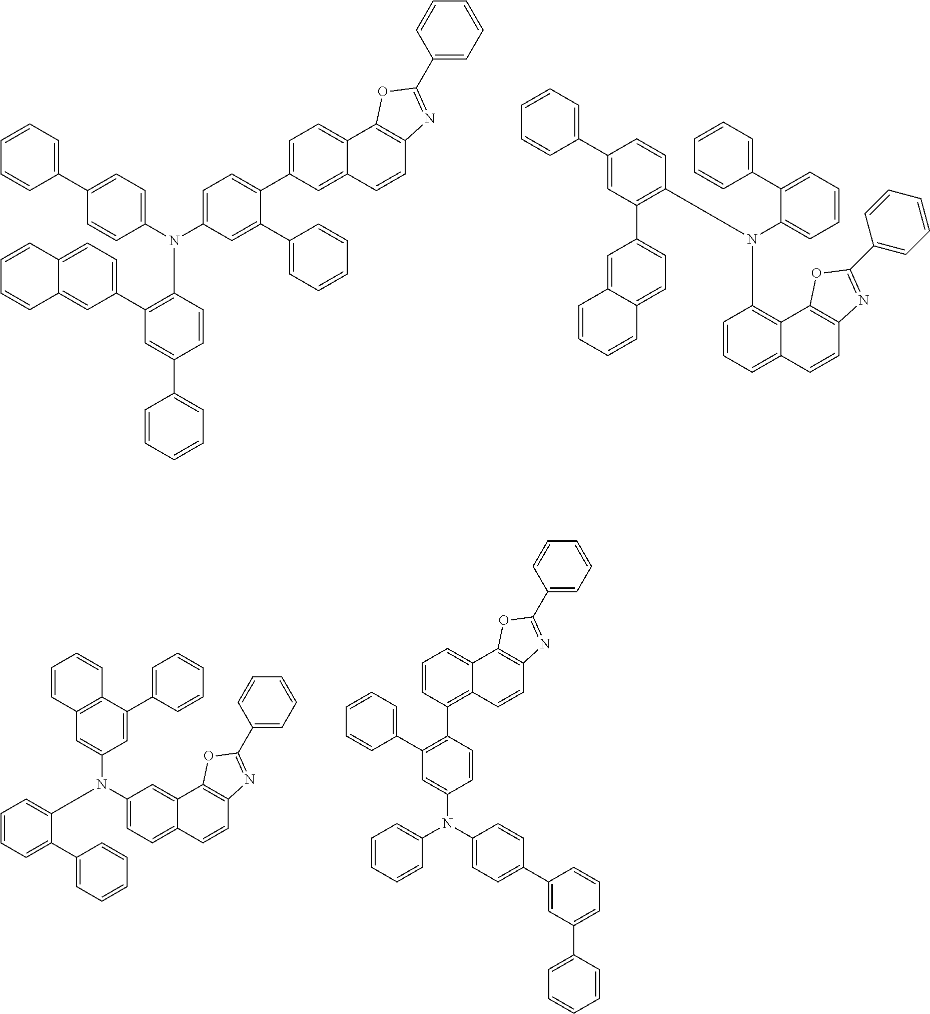 [SMILES: c1ccc(-c2ccc(N(c3ccc(-c4ccc5c(ccc6nc(-c7ccccc7)oc65)c4)c(-c4ccccc4)c3)c3ccc(-c4ccccc4)cc3-c3ccc4ccccc4c3)cc2)cc1 ; c1ccc(-c2ccc(CCN(Cc3cccc4ccc5nc(-c6ccccc6)oc5c34)c3ccccc3-c3ccccc3)c(-c3ccc4ccccc4c3)c2)cc1 ; c1ccc(-c2nc3ccc4ccc(N(Cc5ccccc5-c5ccccc5)c5cc(-c6ccccc6)c6ccccc6c5)cc4c3o2)cc1 ; c1ccc(-c2cccc(-c3ccc(N(c4ccccc4)c4ccc(-c5cccc6c5ccc5nc(-c7ccccc7)oc56)c(-c5ccccc5)c4)cc3)c2)cc1]

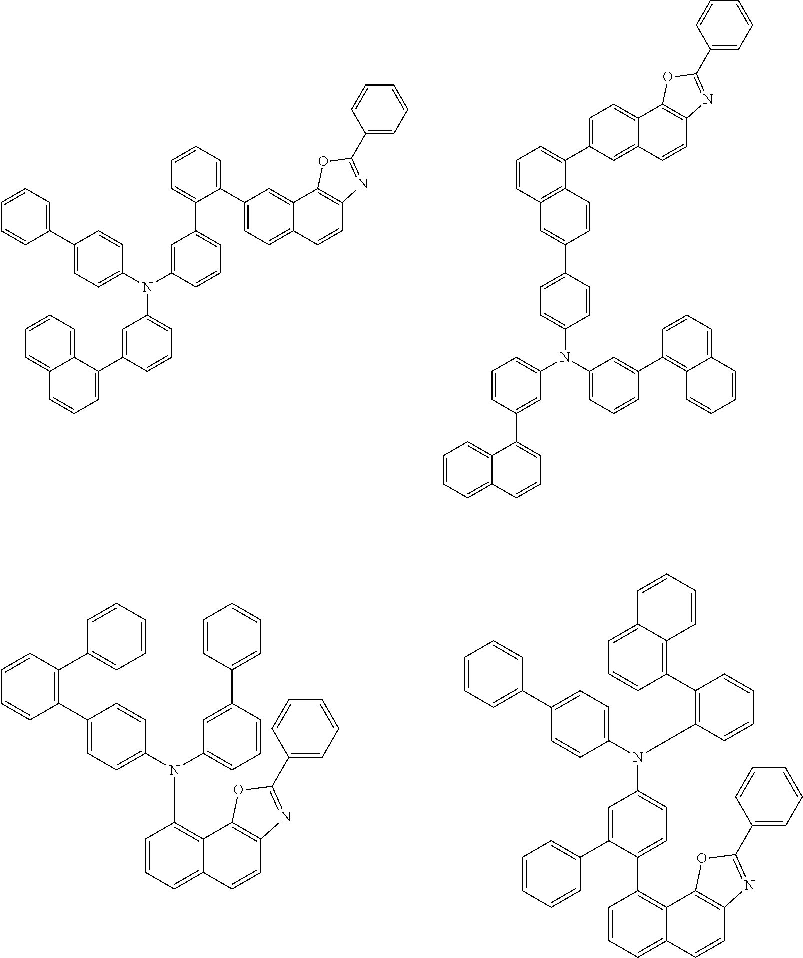 [SMILES: c1ccc(-c2ccc(N(c3cccc(-c4ccccc4-c4ccc5ccc6nc(-c7ccccc7)oc6c5c4)c3)c3cccc(-c4cccc5ccccc45)c3)cc2)cc1 ; c1ccc(-c2nc3ccc4cc(-c5cccc6cc(-c7ccc(N(c8cccc(-c9cccc%10ccccc9%10)c8)c8cccc(-c9cccc%10ccccc9%10)c8)cc7)ccc56)ccc4c3o2)cc1 ; c1ccc(-c2cccc(N(c3ccc(-c4ccccc4-c4ccccc4)cc3)c3cccc4ccc5nc(-c6ccccc6)oc5c34)c2)cc1 ; c1ccc(-c2ccc(N(Cc3ccccc3-c3cccc4ccccc34)c3ccc(-c4cccc5ccc6nc(-c7ccccc7)oc6c45)c(-c4ccccc4)c3)cc2)cc1]

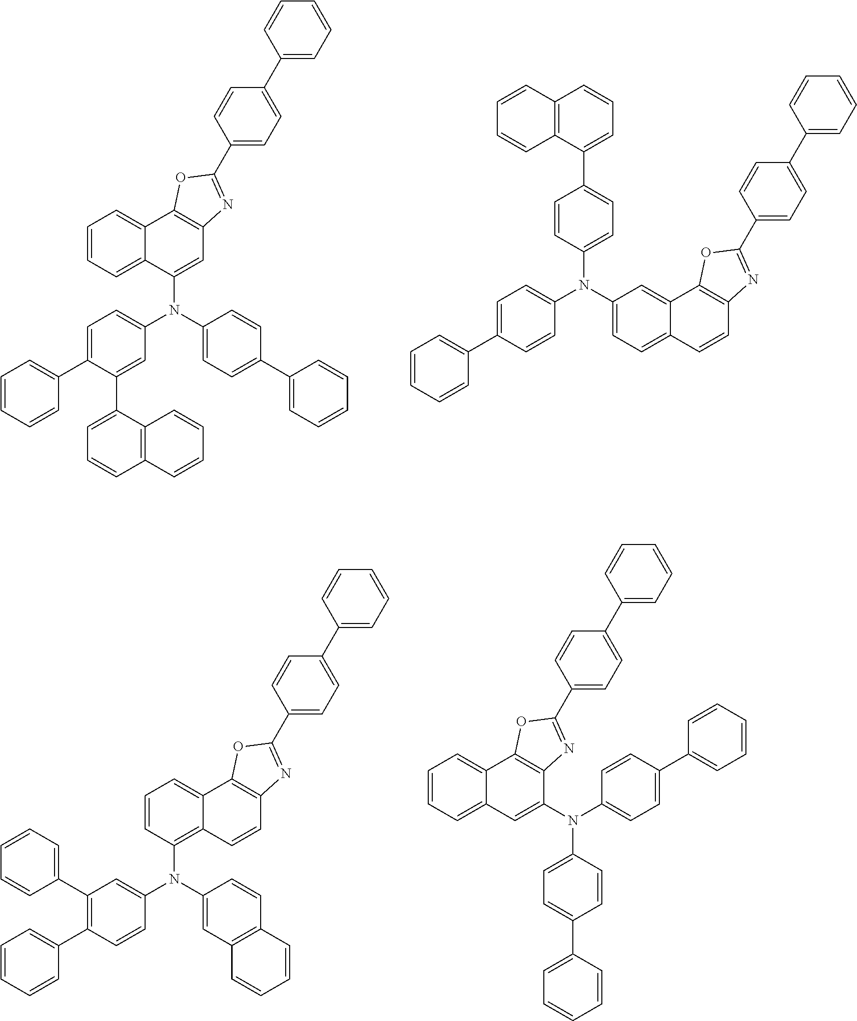 [SMILES: c1ccc(-c2ccc(-c3nc4cc(N(c5ccc(-c6ccccc6)cc5)c5ccc(-c6ccccc6)c(-c6cccc7ccccc67)c5)c5ccccc5c4o3)cc2)cc1 ; c1ccc(-c2ccc(-c3nc4ccc5ccc(N(c6ccc(-c7ccccc7)cc6)c6ccc(-c7cccc8ccccc78)cc6)cc5c4o3)cc2)cc1 ; c1ccc(-c2ccc(-c3nc4ccc5c(N(c6ccc(-c7ccccc7)c(-c7ccccc7)c6)c6ccc7ccccc7c6)cccc5c4o3)cc2)cc1 ; c1ccc(-c2ccc(-c3nc4c(N(c5ccc(-c6ccccc6)cc5)c5ccc(-c6ccccc6)cc5)cc5ccccc5c4o3)cc2)cc1]

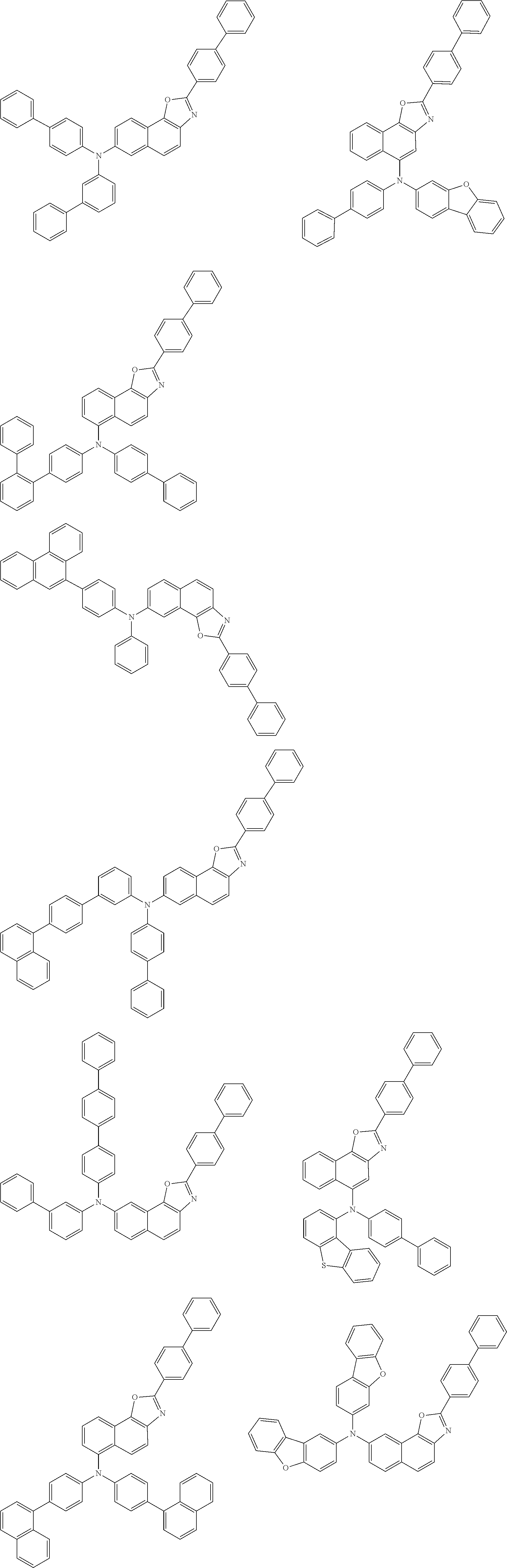

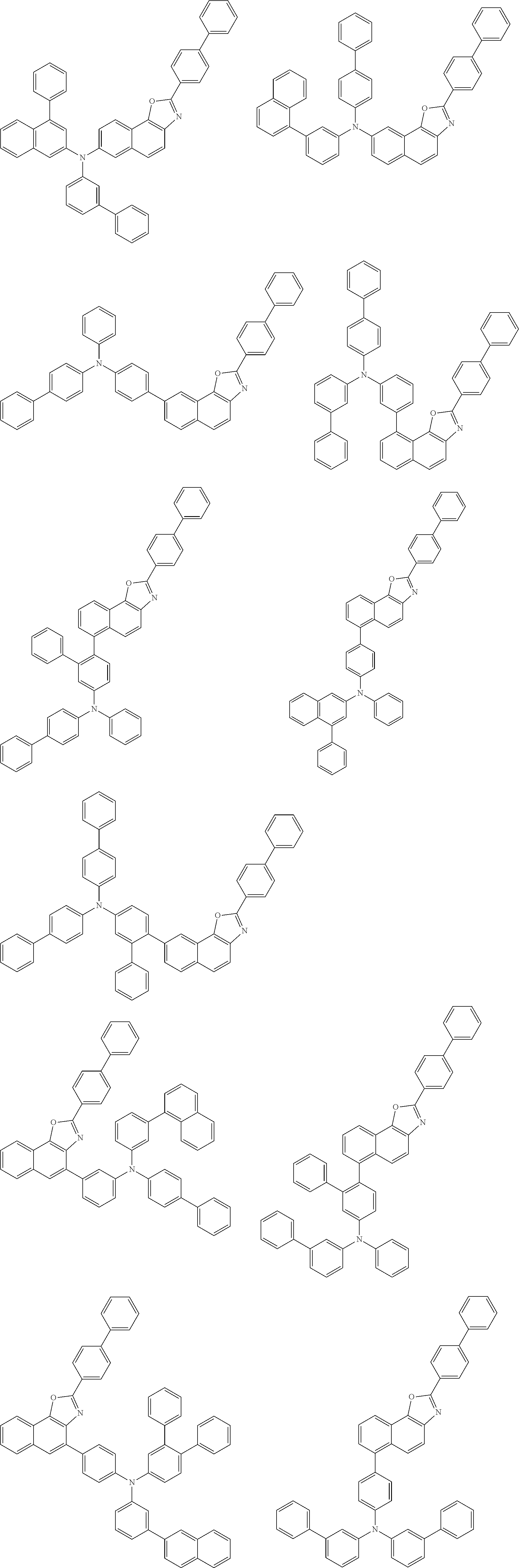

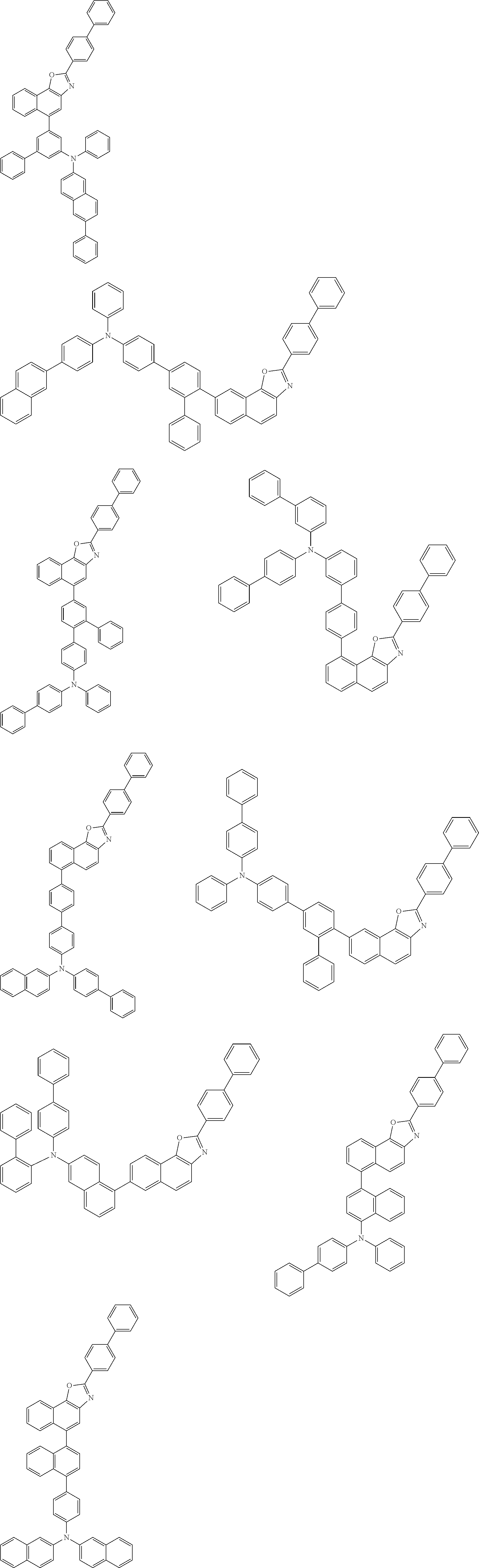

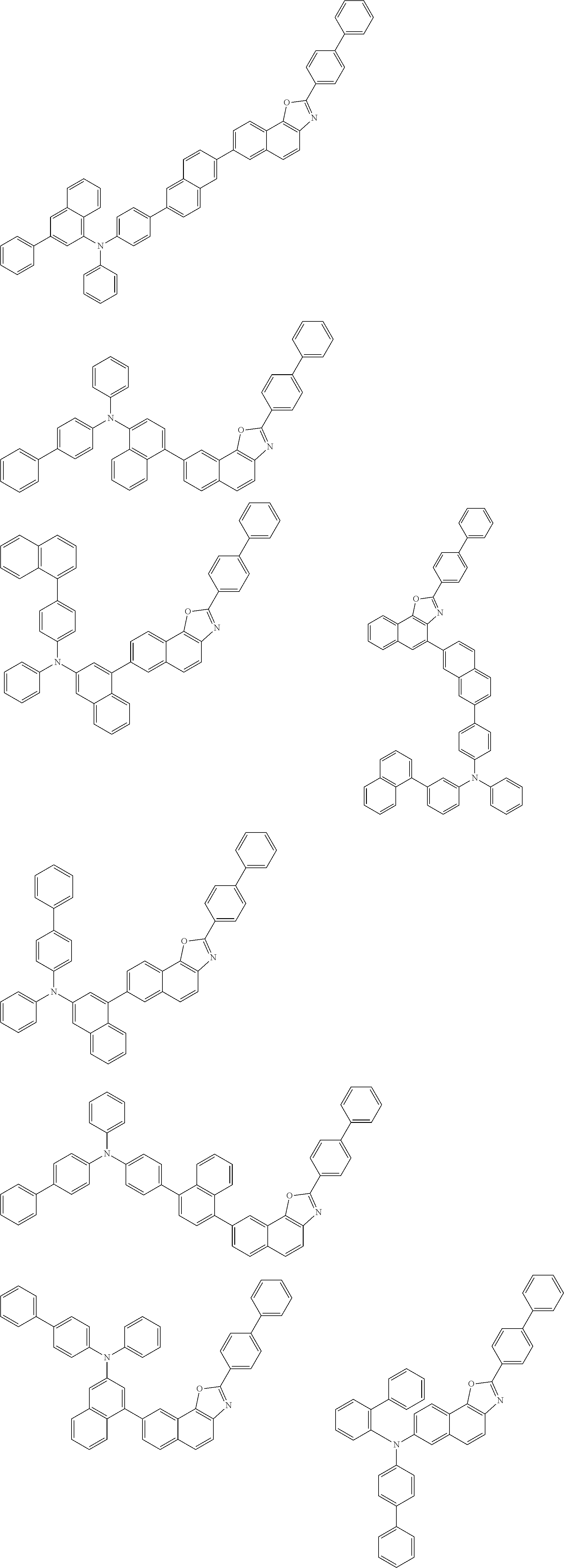

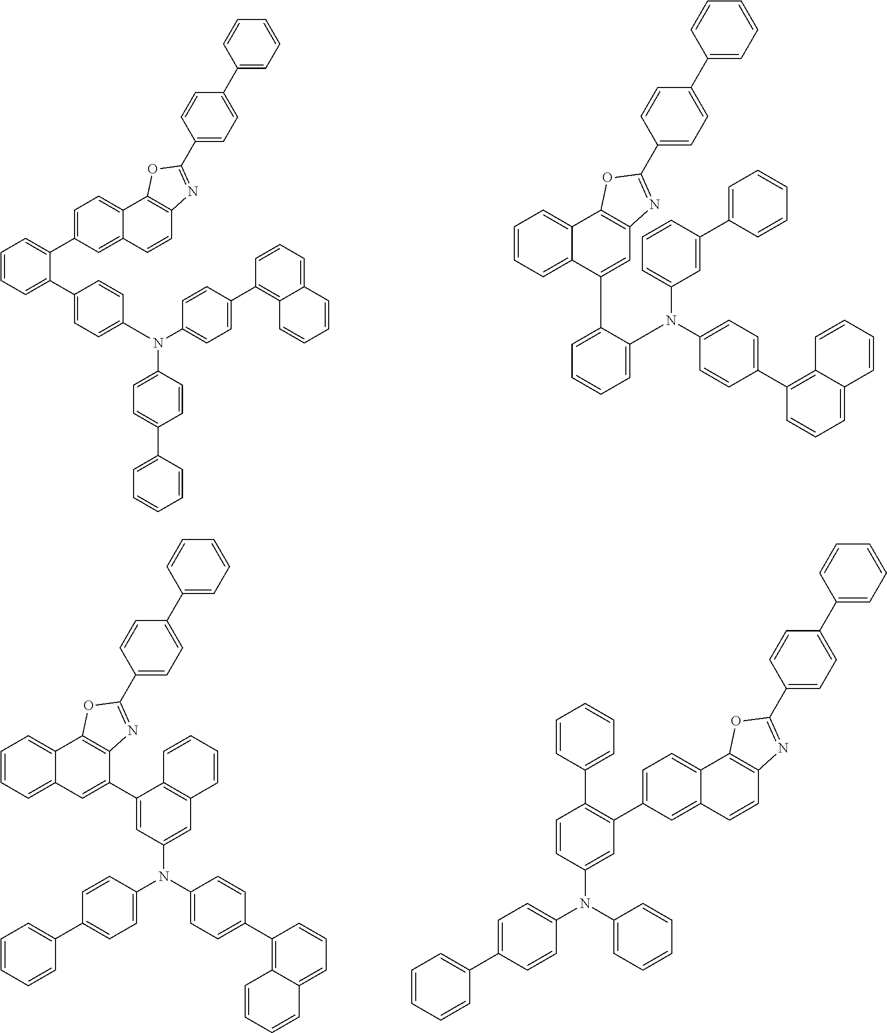

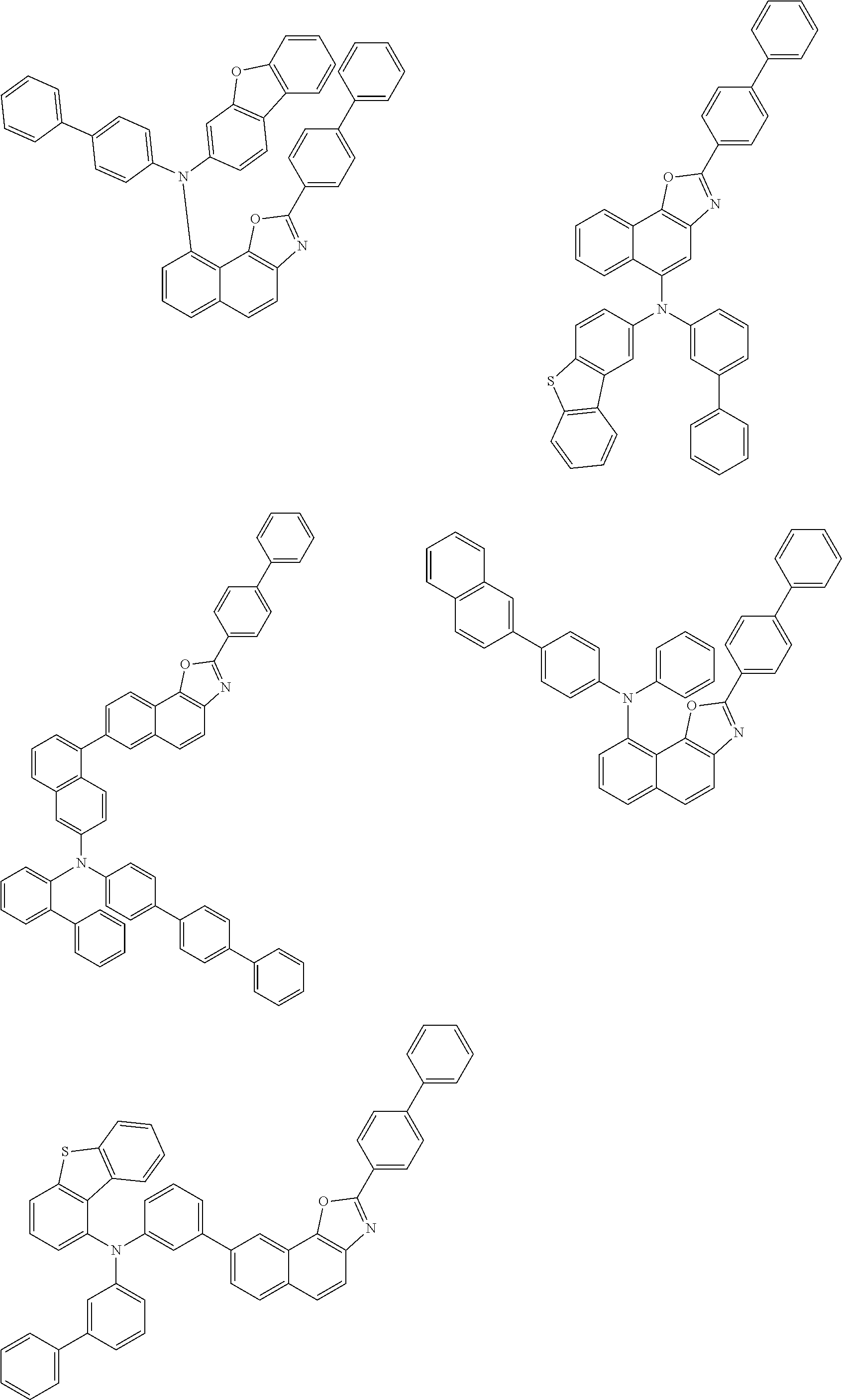 [SMILES: c1ccc(-c2ccc(-c3nc4ccc5cccc(N(c6ccc(-c7ccccc7)cc6)c6ccc7c(c6)oc6ccccc67)c5c4o3)cc2)cc1 ; c1ccc(-c2ccc(-c3nc4cc(N(c5cccc(-c6ccccc6)c5)c5ccc6sc7ccccc7c6c5)c5ccccc5c4o3)cc2)cc1 ; c1ccc(-c2ccc(-c3ccc(N(c4ccc5c(-c6ccc7c(ccc8nc(-c9ccc(-c%10ccccc%10)cc9)oc87)c6)cccc5c4)c4ccccc4-c4ccccc4)cc3)cc2)cc1 ; c1ccc(-c2ccc(-c3nc4ccc5cccc(N(c6ccccc6)c6ccc(-c7ccc8ccccc8c7)cc6)c5c4o3)cc2)cc1 ; c1ccc(-c2ccc(-c3nc4ccc5ccc(-c6cccc(N(c7cccc(-c8ccccc8)c7)c7cccc8sc9ccccc9c78)c6)cc5c4o3)cc2)cc1]

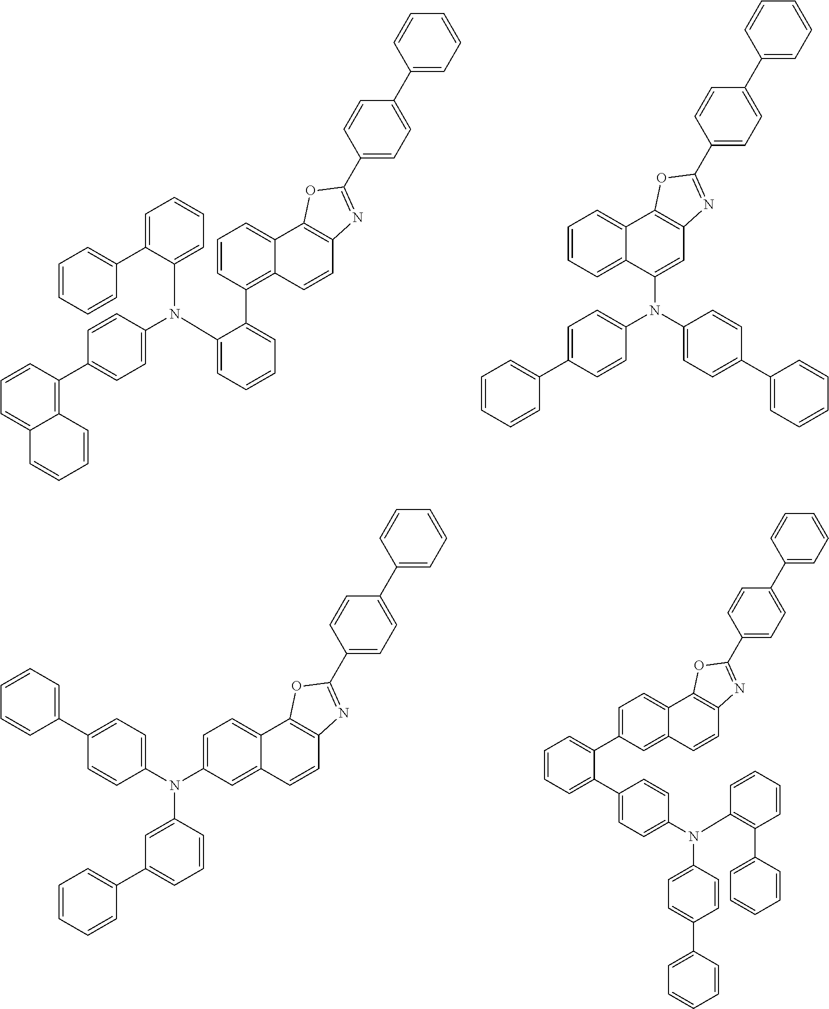 [SMILES: c1ccc(-c2ccc(-c3nc4ccc5c(-c6ccccc6N(c6ccc(-c7cccc8ccccc78)cc6)c6ccccc6-c6ccccc6)cccc5c4o3)cc2)cc1 ; c1ccc(-c2ccc(-c3nc4cc(N(c5ccc(-c6ccccc6)cc5)c5ccc(-c6ccccc6)cc5)c5ccccc5c4o3)cc2)cc1 ; c1ccc(-c2ccc(-c3nc4ccc5cc(N(c6ccc(-c7ccccc7)cc6)c6cccc(-c7ccccc7)c6)ccc5c4o3)cc2)cc1 ; c1ccc(-c2ccc(-c3nc4ccc5cc(-c6ccccc6-c6ccc(N(c7ccc(-c8ccccc8)cc7)c7ccccc7-c7ccccc7)cc6)ccc5c4o3)cc2)cc1]

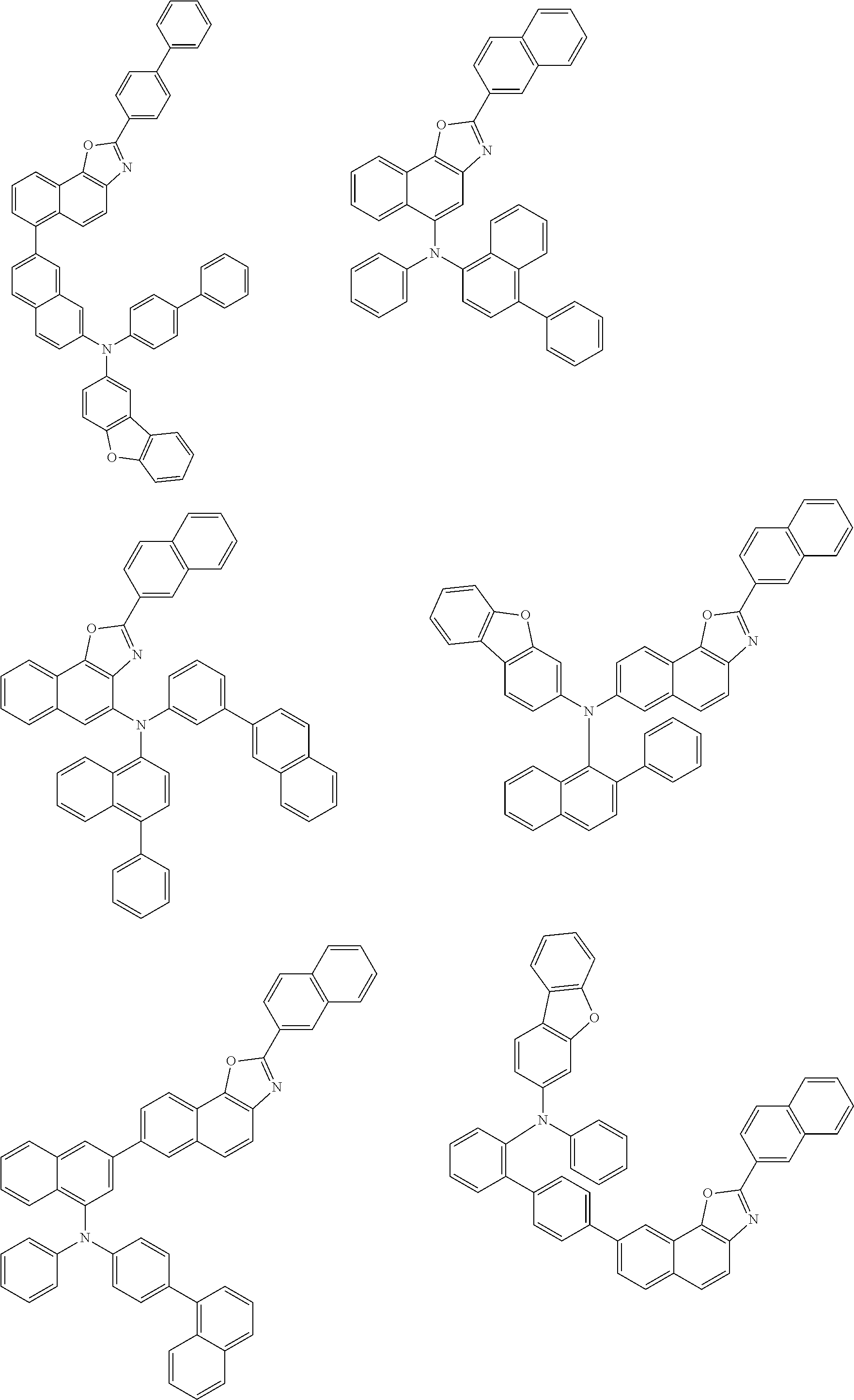

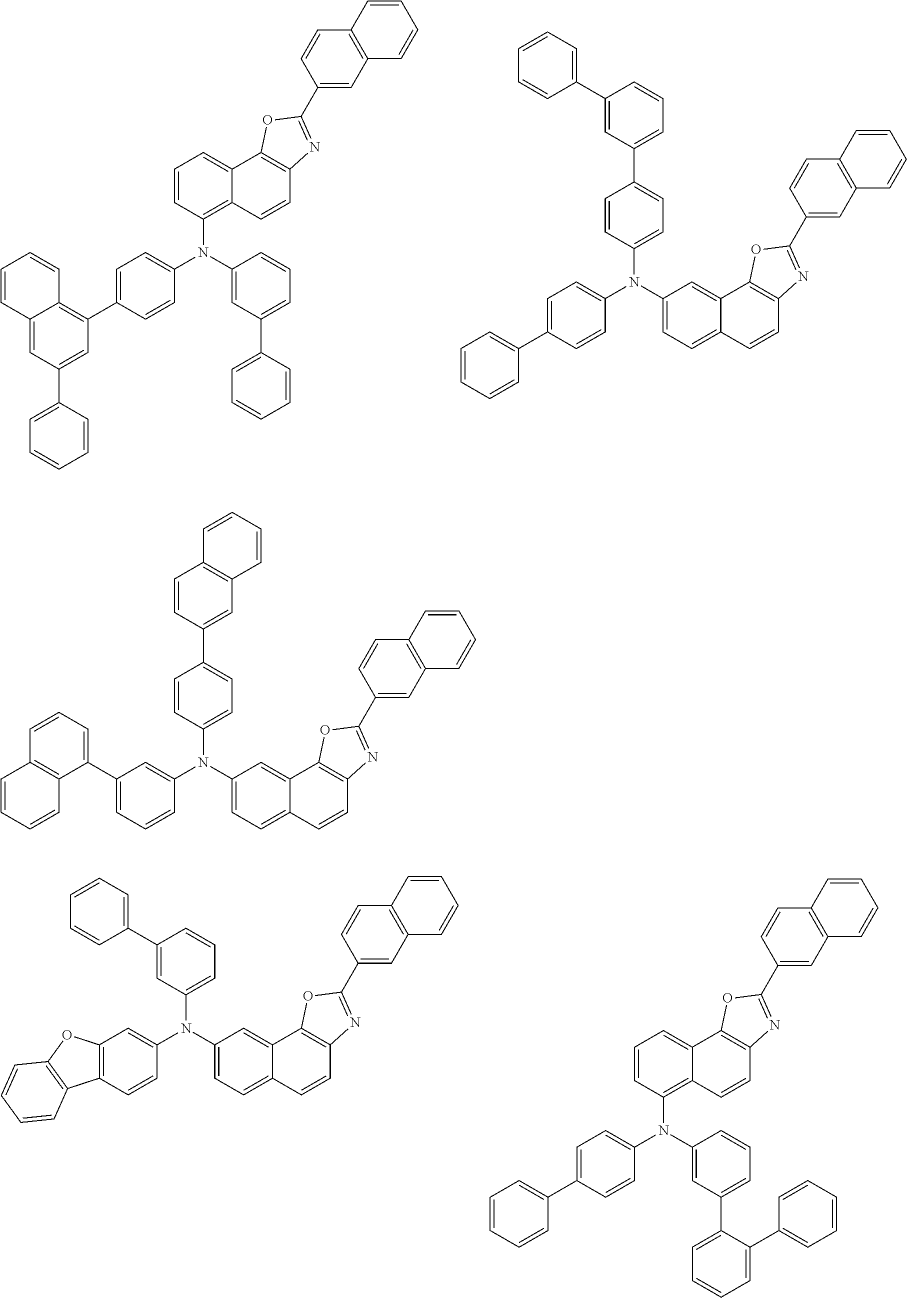 [SMILES: c1ccc(-c2cccc(N(c3ccc(-c4cc(-c5ccccc5)cc5ccccc45)cc3)c3cccc4c3ccc3nc(-c5ccc6ccccc6c5)oc34)c2)cc1 ; c1ccc(-c2ccc(N(c3ccc(-c4cccc(-c5ccccc5)c4)cc3)c3ccc4ccc5nc(-c6ccc7ccccc7c6)oc5c4c3)cc2)cc1 ; c1cc(-c2cccc3ccccc23)cc(N(c2ccc(-c3ccc4ccccc4c3)cc2)c2ccc3ccc4nc(-c5ccc6ccccc6c5)oc4c3c2)c1 ; c1ccc(-c2cccc(N(c3ccc4c(c3)oc3ccccc34)c3ccc4ccc5nc(-c6ccc7ccccc7c6)oc5c4c3)c2)cc1 ; c1ccc(-c2ccc(N(c3cccc(-c4ccccc4-c4ccccc4)c3)c3cccc4c3ccc3nc(-c5ccc6ccccc6c5)oc34)cc2)cc1]

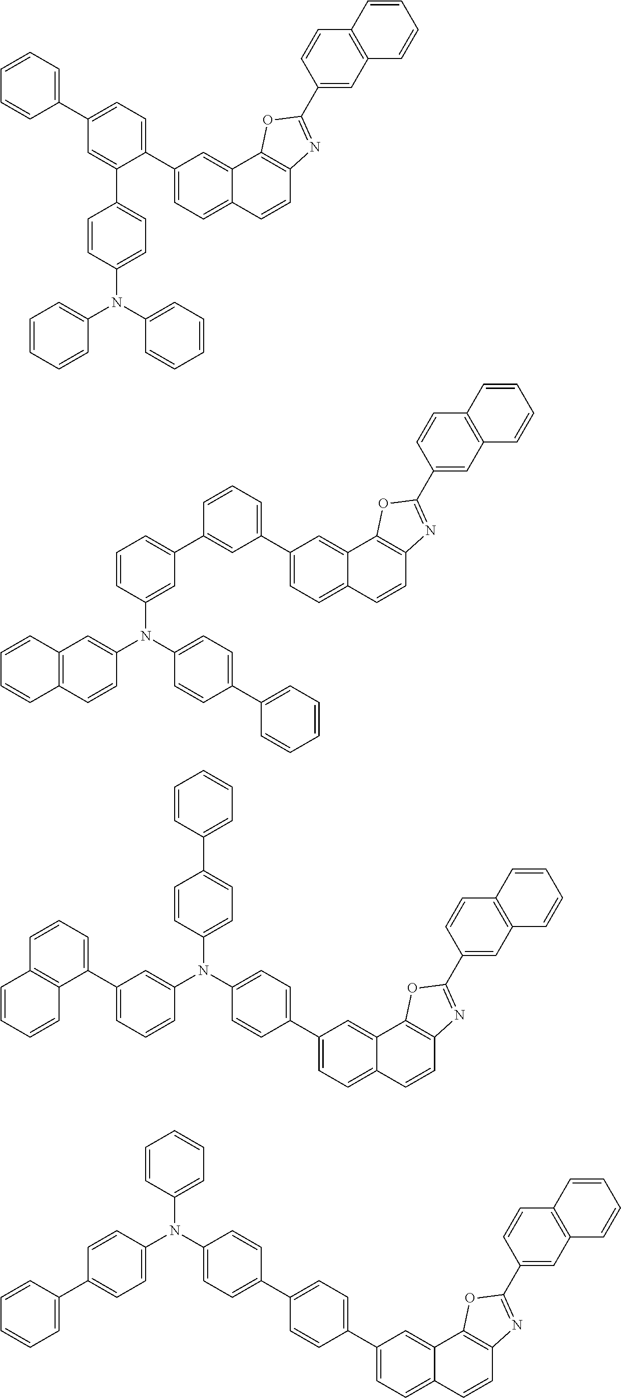 [SMILES: c1ccc(-c2ccc(-c3ccc4ccc5nc(-c6ccc7ccccc7c6)oc5c4c3)c(-c3ccc(N(c4ccccc4)c4ccccc4)cc3)c2)cc1 ; c1ccc(-c2ccc(N(c3cccc(-c4cccc(-c5ccc6ccc7nc(-c8ccc9ccccc9c8)oc7c6c5)c4)c3)c3ccc4ccccc4c3)cc2)cc1 ; c1ccc(-c2ccc(N(c3ccc(-c4ccc5ccc6nc(-c7ccc8ccccc8c7)oc6c5c4)cc3)c3cccc(-c4cccc5ccccc45)c3)cc2)cc1 ; c1ccc(-c2ccc(N(c3ccccc3)c3ccc(-c4ccc(-c5ccc6ccc7nc(-c8ccc9ccccc9c8)oc7c6c5)cc4)cc3)cc2)cc1]

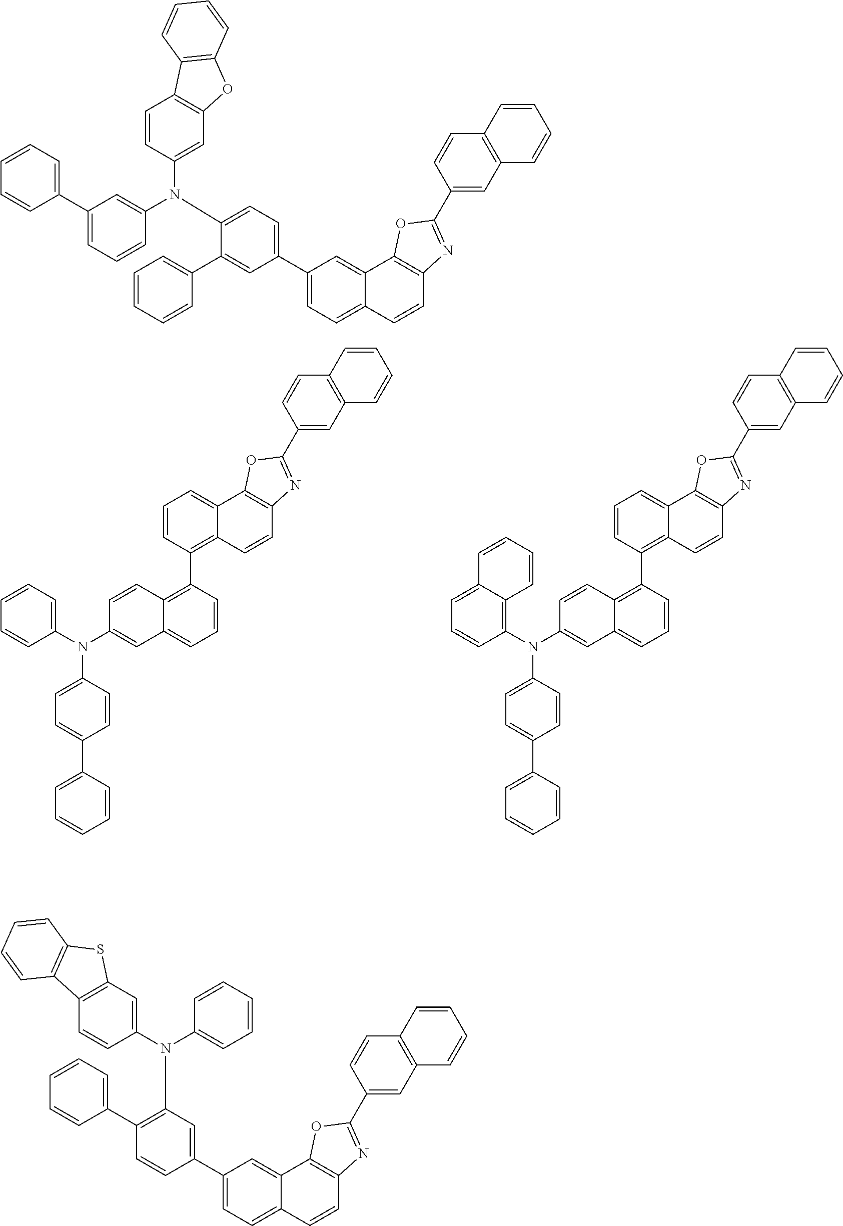

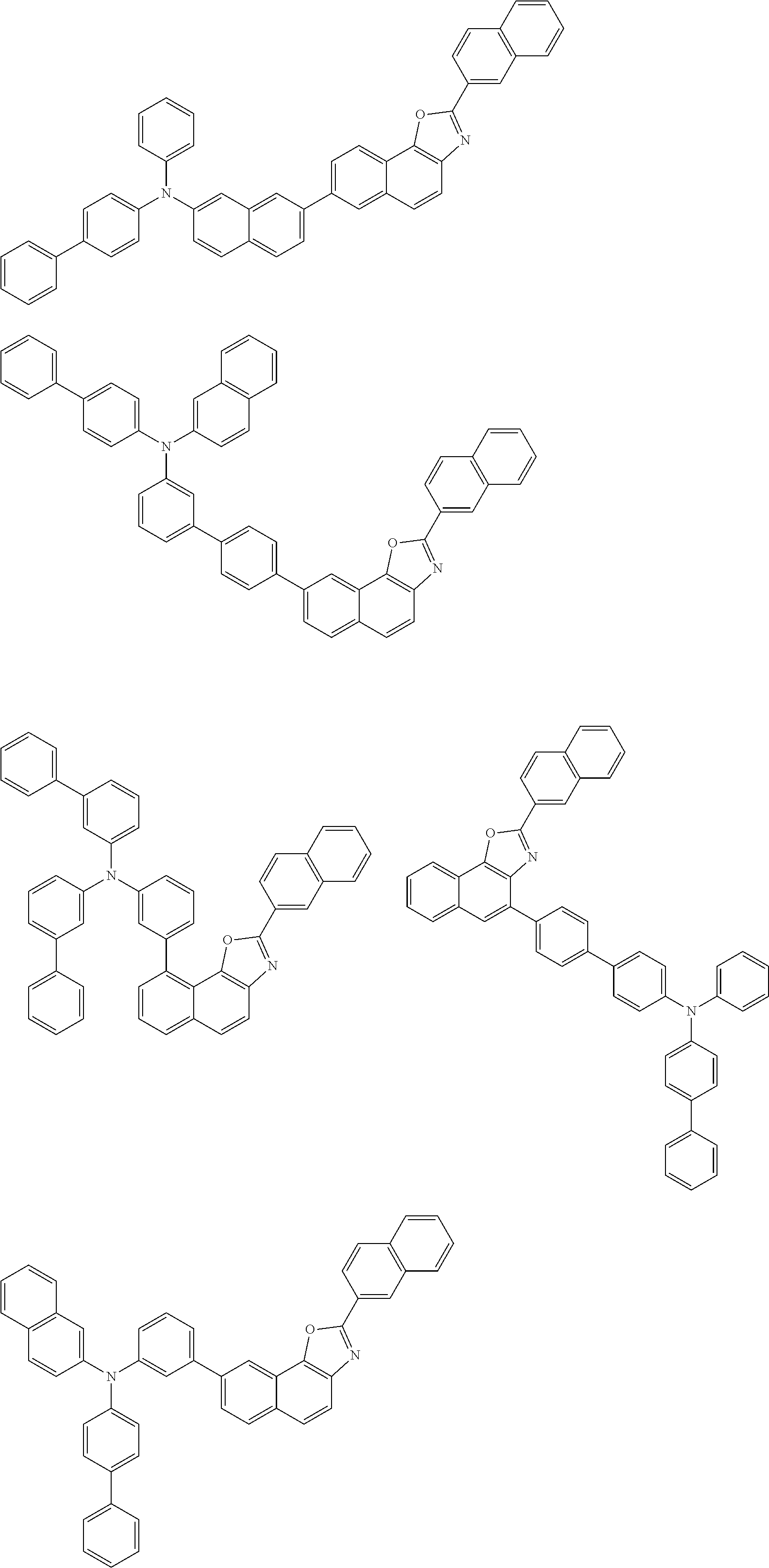

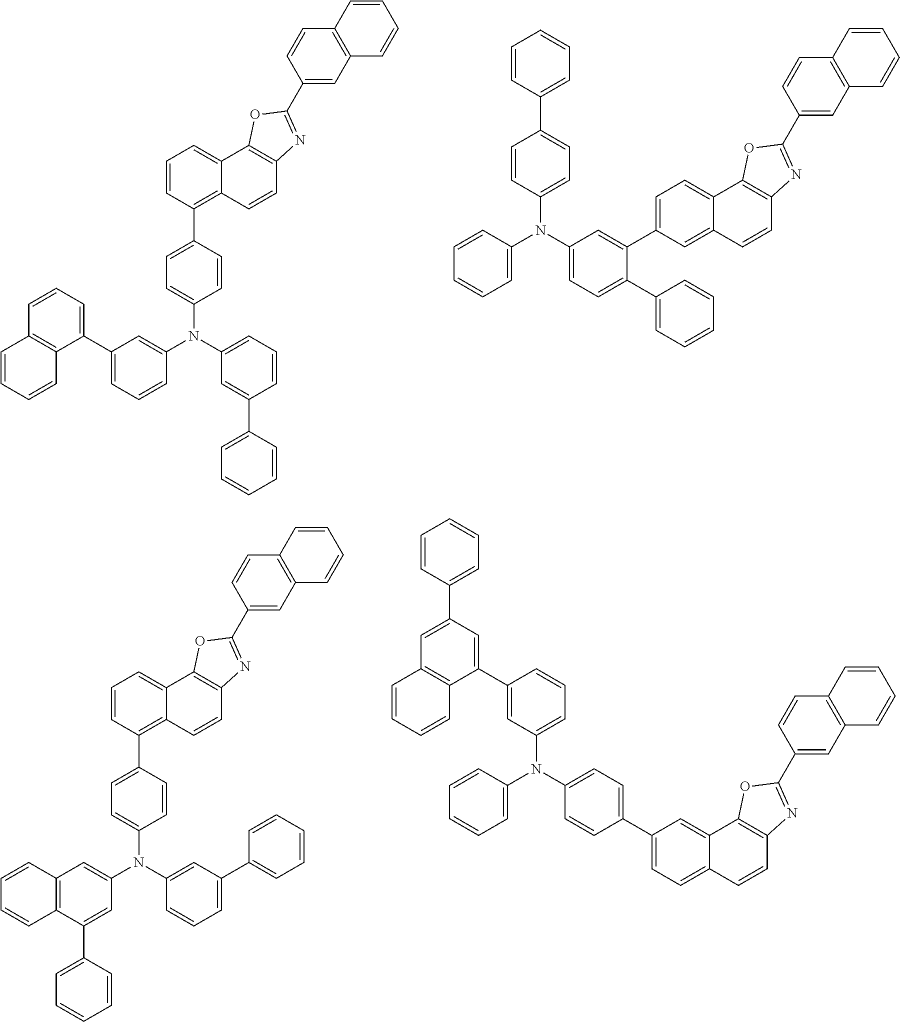 [SMILES: c1ccc(-c2cccc(N(c3ccc(-c4cccc5c4ccc4nc(-c6ccc7ccccc7c6)oc45)cc3)c3cccc(-c4cccc5ccccc45)c3)c2)cc1 ; c1ccc(-c2ccc(N(c3ccccc3)c3ccc(-c4ccccc4)c(-c4ccc5c(ccc6nc(-c7ccc8ccccc8c7)oc65)c4)c3)cc2)cc1 ; c1ccc(-c2cccc(N(c3ccc(-c4cccc5c4ccc4nc(-c6ccc7ccccc7c6)oc45)cc3)c3cc(-c4ccccc4)c4ccccc4c3)c2)cc1 ; c1ccc(-c2cc(-c3cccc(N(c4ccccc4)c4ccc(-c5ccc6ccc7nc(-c8ccc9ccccc9c8)oc7c6c5)cc4)c3)c3ccccc3c2)cc1]

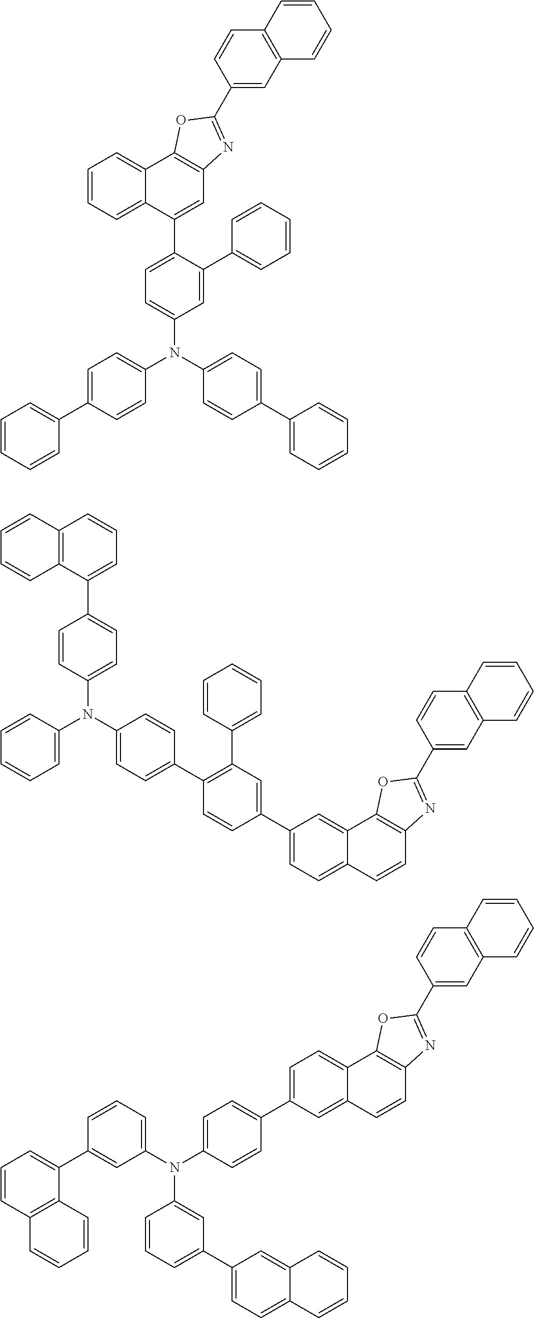

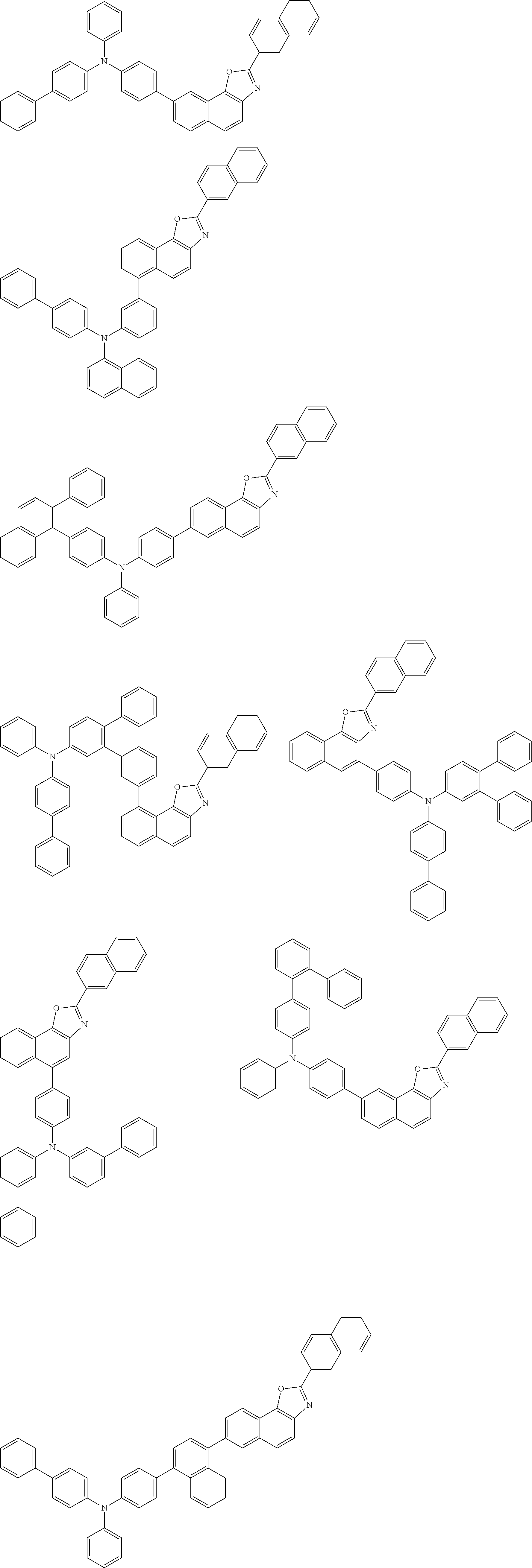

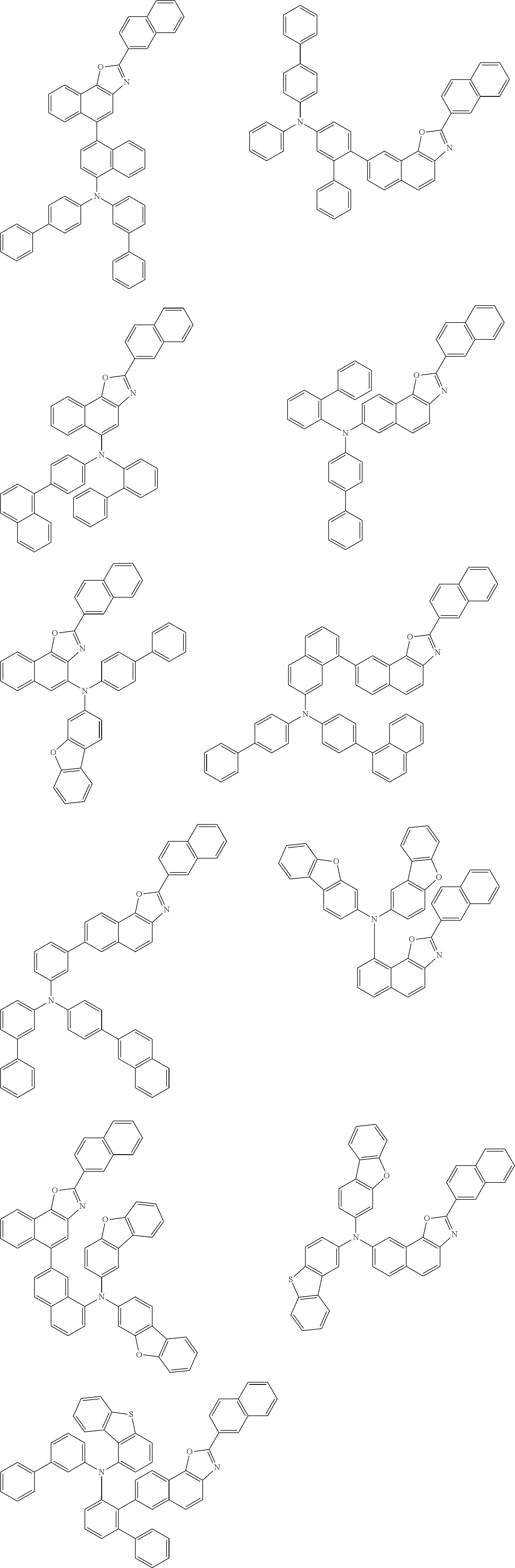

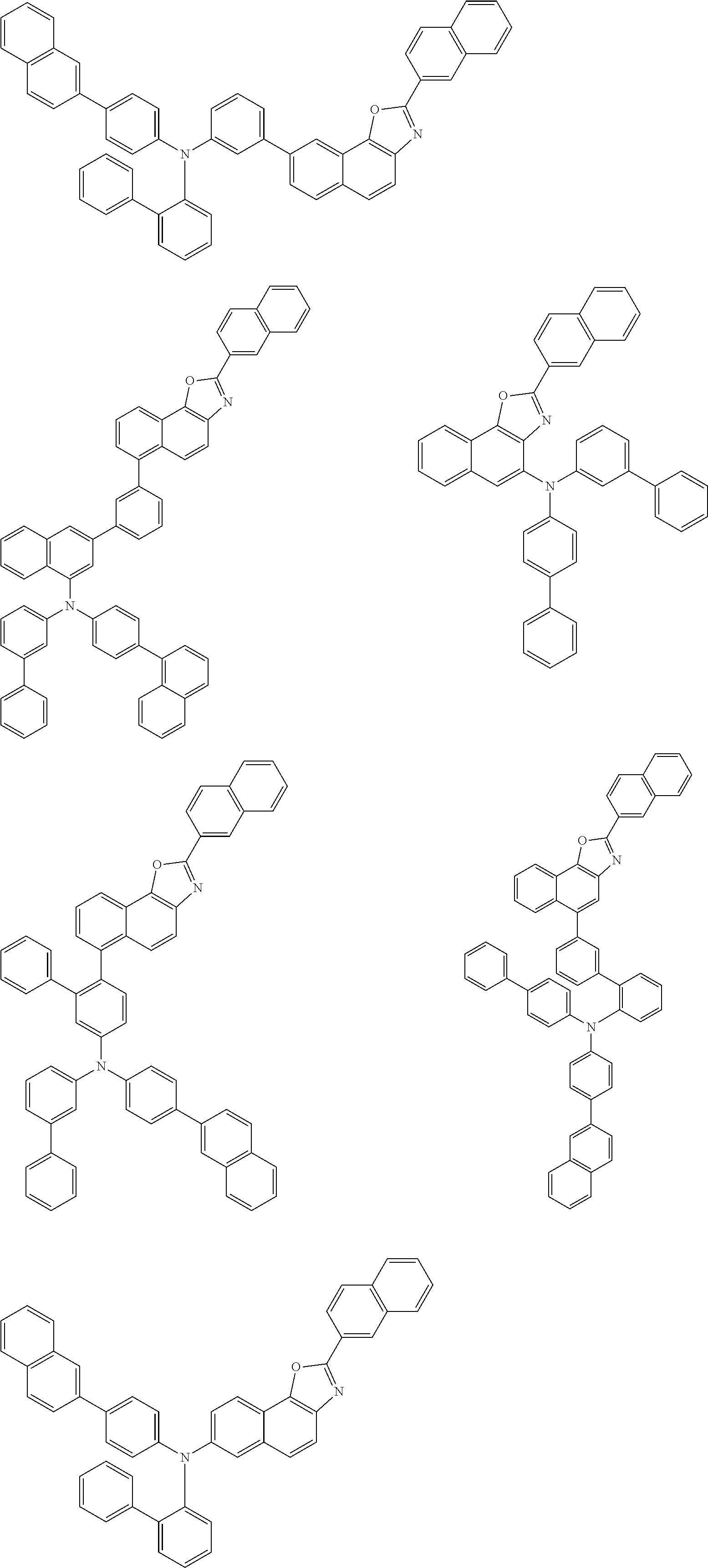

11. An organic light emitting device comprising:
a first electrode;
a second electrode disposed to face the first electrode; and
an organic material laver including one or more layers between the first electrode and the second electrode,
wherein one or more layers of the organic material layer comprise the compound according to claim 1.

12. The organic light emitting device of claim 11,
wherein the organic material layer comprises an electron blocking layer, and
the electron blocking laver comprises the compound.
